(19) United States
(12) Patent Application Publication
(10) Pub. No.: US 2004/0241653 A1
(43) Pub. Date: Dec. 2, 2004

(54) Methods for identifying marker genes for cancer

(76) Inventors: Elena Feinstein, Rehovot (IL); Andrei V. Gudkov, Gates Mills, OH (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/335,053

(22) Filed: Dec. 31, 2002

Related U.S. Application Data
(60) Provisional application No. 60/345,317, filed on Dec. 31, 2001.

(51) Int. Cl. C12Q 1/68; G01N 33/574

(52) U.S. Cl. 435/6; 435/7.23

(57) ABSTRACT

The invention describes a method of identifying tissue-specific tumor markers, and diagnostic and therapeutic methods and compositions of using the same. More specifically, the invention presents a method for a rational search of diagnostic and prognostic cancer markers and therapeutic targets among the genes negatively regulated by tumor suppressor genes.

### FIELD OF THE INVENTION

The invention relates generally to the field of cancer. More specifically, the invention details methods for the identification of markers specific for one or several types of cancer, depending on tissue origin. Such markers are useful in numerous diagnostic and prognostic applications as well as cancer type-specific targets for therapeutic intervention.

### BACKGROUND OF THE INVENTION

An effective cure of any given cancer will greatly depend on the development of new diagnostic assays based on identification of reliable serological and histological markers and on designing new therapeutic strategies and pharmaceuticals for effective elimination of cancer cells in the diseased individual. Tumor suppressor genes normally function to inhibit division or survival of genetically damaged cells and thus function to prevent the development of tumors. Mutations in tumor suppressor genes cause the cell to ignore one or more of the components of a network of inhibitory signals, removing the inhibitory mechanisms from the cell cycle, and resulting in a higher rate of uncontrolled growth, i.e., cancer. Tumor suppressor genes are defined by the impact of their absence and thus tend to be recessive. Thus, neoplasia is the result of the loss of function of these genes. The loss or inactivation of a normal tumor suppressor gene may be acquired somatically in a single clone of cells or be constitutionally present throughout the body, including the germ line.

There are numerous tumor suppressors known to those of skill in the art, including, for example, p53; the retinoblastoma gene, commonly referred to as Rb1; the adenomatous polyposis of the colon gene (APC); familial breast/ovarian cancer gene I (BRCA1); familial breast/ovarian cancer gene 2 (BRCA2); CDH1 cadherin 1 (epithelial cadherin or E-cadherin) gene; cyclin-dependent kinase inhibitor 1C gene (CDKN1C, also known as p57, KIP2 or BWS); cyclin-dependent kinase inhibitor 2A gene (CDKN2A also known as p16 MTS1 (multiple tumor suppressor 1), TP16 or INK4); familial cylindromatosis gene (CYLD; formerly known as EAC (epithelioma adenoides cysticum)); E1A-binding protein gene (p300); multiple exostosis type 1 gene (EXT1); multiple exostosis type 2 gene (EXT2); homolog of Drosophila mothers against decapentaplegic 4 gene (MADH4; formerly referred to as DPC4 (deleted in pancreatic carcinoma 4) or SMAD4 (SMA- and MAD-related protein 4)); mitogen-activated protein kinase kinase 4 (MAP2K4; also referred to as JNKK1, MEK4, MKK4, or PRKMK4; formerly known as SEK1 or SERK1); multiple endocrine neoplasia type 1 gene (MEN1); homolog of E. coli MutL gene (MLH1 also known as HNPCC (hereditary non-polyposis colorectal cancer) or HNPCC2; formerly referred to as COCA2 (colorectal cancer 2) and FCC2); homolog of E. coli MutS 2 gene (MSH2 also called HNPCC (hereditary non-polyposis colorectal cancer) or HNPCC1 and formerly known as COCA1 (colorectal cancer 1) and FCC1); neurofibromatosis type 1 gene (NF1); neurofibromatosis type 2 gene (NF2); protein kinase A type 1, alpha, regulatory subunit gene (PRKAR1A, formerly known as PRKAR1 or TSE1 (tissue-specific extinguisher 1)); homolog of Drosophila patched gene (PTCH; also called BCNS); phosphatase and tensin homolog gene (PTEN, also called MMAC1 (mutated in multiple advanced cancers 1), formerly known as BZS (Bannayan-Zonana syndrome) and MHAM1 (multiple hamartoma 1)); succinate dehydrogenase cytochrome B small subunit gene (SDHD; also called SDH4); Swi/Snf5 matrix-associated actin-dependent regulator of chromatin gene (SMARCB1, also referred to as BAF47, HSNFS, SNF5/INI1, SNF5L1, STH1P, and SNR1); serine/threonine kinase 11 gene (STK11 also known as LKB1 and PJS); tuberous sclerosis type 1 gene (TSC1 also known as KIAA023); tuberous sclerosis type 2 gene (TSC2, previously referred to as TSC4); von Hipple-Lindau syndrome gene (VHL); and Wilms tumor 1 gene (WT1, formerly referred to as GUD (genitourinary dysplasia), WAGR (Wilms tumor, aniridia, genitourinary abnormalities, and mental retardation), or WIT-2), DAP-kinase, FHIT, Werner syndrome gene, and Bloom syndrome gene.

The p53 tumor suppressor gene is an exemplary tumor suppressor in its mode of action. It encodes a nuclear transcription factor that accumulates in cells in response to a variety of stresses, thereby inducing growth arrest or apoptosis (Gottlieb and Oren, Biochim Biophys Acta., 1287(2-3):77-102 (1996). p53 or the pathway mediated by p53 are inactivated in the majority of human tumors, including advanced prostate cancer (Steele et al., Br J Surg., 85(11):1460-1467 (1998); Ozen and Pathak, Anticancer Res., 20(3B):1905-1912 (2000).

One of the functions of the p53 protein in the cell is that it binds DNA stimulating the expression of p21-waf1 that interacts with a cell division-stimulating protein (cdk2). When p21 is complexed with cdk2, the cell cannot pass to the S stage of cell division (G1 check point). Mutant p53 can no longer bind DNA in an effective way, and as a consequence the p21-waf1 protein is not made available to act as the ‘stop signal’ for cell division. Thus, cells divide uncontrollably and form tumors. Thus, inactivation of p53 is associated with the loss of this cell cycle checkpoint control and with the consequent resistance to anti-cancer treatment, genomic instability, and enhanced angiogenesis, leading to rapid tumor progression (Gottlieb and Oren, Biochim Biophys Acta., 1287(2-3):77-102 (1996); Cordon-Cardo et al., Semin. Surg. Oncol., 13:319-327 (1997).

Many p53-mediated effects are achieved through the activity of p53-responsive genes that are either up- or down-regulated by p53. In fact, the activity of p53-responsive genes account, in part, for p53-mediated checkpoint control [upregulation of p21-waf1, 14-3-3 □(G2 checkpoint)], apoptosis (upregulation of bax, PUMA, and genes determining enhanced reactive oxygen species metabolism), suppression of angiogenesis (upregulation of thrompospondins 1 and 2, and downregulation of VEGF) and p53 feedback regulation (upregulation of mdm2) (see Gottlieb and Oren, Biochim Biophys Acta., 1287(2-3):77-102 (1996) for references). Much like p53, the other tumor suppressors listed above also mediate their effects through the activity of responsive genes that are either up- or down-regulated by the tumor suppressor, directly or indirectly. Genes that have altered expression in tumors may serve as targets for development of anti-cancer drugs, or cancer markers or both. However, the relationship between changes in gene expression, resulting from tumor suppressor deficiency, and tumor progression is not sufficiently understood. It also remains unclear why the germline loss of tumor suppressor gene function leads to development of certain specific types of cancer and not others. This implies that in each specific tissue the changes in gene expression imposed by the loss of tumor suppressor gene are unique. Taking into account a need for tissue-specific markers of cancer, the inventors have devised a method for the identification of such tissue-specific markers by exploiting the tumor suppressor regulation of genes in cancer cells.

### SUMMARY OF THE INVENTION

The invention relates to methods for diagnosing and prognosing cancer by utilizing general as well as tissue-specific genetic markers, methods for identifying these markers, and the markers identified by such methods.

The invention provides a method of identifying tissue-specific and general tumor markers and diagnostic and therapeutic methods and compositions of using the same. Diagnostic markers may be screening markers (secreted polypeptides), histological markers (using which it is possible to distinguish tumor tissue from benign tissue within histological samples) or staging markers (determining the stage of a cancer by detection of the presence of specific cancer cells in blood (micrometastases) by RT-PCR on identified cancer-type-specific markers on the whole blood RNA).

The invention provides a method of identifying a diagnostic marker for a cancer comprising: a) obtaining a first cell from a first cell type of the cancer, the cell comprising a defective tumor suppressor expression; b) obtaining a second cell of the first cell type, wherein the second cell comprises a wild-type tumor suppressor expression; c) identifying genes having an increased level of expression in the first cell as compared to the second cell; and d) selecting at least one gene of step c) as a diagnostic marker for the cancer.

In the diagnostic and therapeutic methods for using such a marker(s), the invention provides a method of diagnosing a cancer in a subject comprising determining, in a sample from the subject, the level of at least one polypeptide, wherein a higher level of the polypeptide compared to the level of the polypeptide in a subject free of cancer is indicative of cancer, and wherein the polypeptide is selected from the group consisting of a) polypeptides encoded by the polynucleotides listed in Table 5 or in Table 6; and b) polypeptides which are at least 70% homologous to the polypeptides of a) at the amino acid sequence level. In one embodiment of the diagnostic methods, the level of a polypeptide-encoding polynucleotide is determined, rather than the polypeptide itself. In such methods, the invention contemplates any of the polynucleotides in Table 6, polynucleotides having sequences that differ from the polynucleotides in a) without changing the polypeptide encoded thereby, and polynucleotides that are at least 70% homologous to the polynucleotides of a) at the nucleic acid sequence level.

In the case of at least p53 and possibly other tumor suppressors, the invention further provides a method of determining the p53 status within the tumor (i.e., whether the cancer cell is a p53⁻ or a p53⁺ tumor cell.), which is important for prognosis and treatment selection.

The invention also provides a method for monitoring the activity of p53 suppressive drugs, or drugs that suppress other tumor suppressors described herein, by measuring any of the markers identified herein the polypeptides of which are secreted, such as PSA or pancreatitis-associated protein. In this aspect, the invention provides a method of measuring the responsiveness of a subject to a cancer treatment comprising determining the level of at least one polypeptide in a sample taken from the subject before treatment, and comparing it with the level of the polypeptide in a sample taken from the subject after treatment, a decrease in the level indicating responsiveness of the subject to the cancer treatment, wherein the polypeptide is selected from the group consisting of a) polypeptides encoded by the polynucleotides listed in Table 5 and Table 6; and b) polypeptides which are at least 70% homologous to the polypeptides of a).

In a related aspect, the invention provides a method of measuring the responsiveness of a subject to a cancer treatment comprising determining the level of at least one polypeptide-encoding polynucleotide in a sample taken from the subject before treatment, and comparing it with the level of the polynucleotide in a sample taken from the subject after treatment, a decrease in the level indicating responsiveness of the subject to the cancer treatment, wherein the polynucleotide is selected from the group consisting of a) the polynucleotides listed in Table 6; b) polynucleotides having sequences that differ from the polynucleotides in a), without changing the polypeptide encoded thereby; and c) polynucleotides which are at least 70% homologous to the polynucleotides of a).

According to another aspect of the invention, a method is provided of screening for drugs useful in the treatment of cancer. A cell which harbors a tumor suppressor mutation or defective expression is contacted with a test substance. Expression of a transcript or its translation product is monitored. The transcript is a tissue-specific tumor marker of the invention. A test substance is identified as a potential drug for treating cancer if it decreases expression of a marker identified as one that is up-regulated as a result of loss of tumor suppressor function. Alternatively, the test substance is identified as a potential drug if it increases the expression of a marker identified as one that is down regulated as a result of loss of tumor suppressor function.

For example, the invention provides a method for screening for compounds that modulate the activity of a tumor suppressor gene comprising a) obtaining a cell comprising a defective tumor suppressor expression; b) measuring the level of expression of a marker of Table 5 or 6 in the cell; c) contacting the cell with a test compound; and d) measuring the expression of the marker of step b) after the contacting step c), wherein a change in the level of expression after the contacting step as compared to the level of expression before the contacting step is indicative of the ability of the compound to modulate the activity of the tumor suppressor gene.

Another aspect of the invention concerns a method of determining p53 inactivation in prostate cells of an individual comprising determining the levels of serum PSA, wherein elevated serum PSA levels in said individual are indicative of p53 inactivation in said prostate cells.

Yet another aspect of the invention a method of monitoring the effect of a p53-based cancer therapy on a prostate cancer patient, the prostate cancer cells of said patient having a p53 loss of function mutation, said method comprising determining the levels of serum PSA of said patient before and after said therapy, wherein a decrease in the serum PSA levels after provision of said therapy is indicative of said therapy overcoming the deleterious effects of said p53 mutation.

In addition, the invention also contemplates a method of detection of p53 inactivation in other tissues. For example, from observations of pancreatitis associated protein, the inactivation of p53 in pancreatic carcinoma may be determined. Similar observations may be made about other preferred markers disclosed in this application.

Numerous other aspects and advantages of the invention will be apparent upon consideration of the following drawings and detailed description.

### BRIEF DESCRIPTION OF THE DRAWINGS

The following drawings form part of the present specification and are included to further illustrate aspects of the invention. The invention may be better understood by reference to the drawings in combination with the detailed description of the preferred embodiments presented herein.

FIG. 1: Inactivation of p53 pathway in LNCaP cells by GSE56 correlates with increased secretion of PSA. Quantitation of PSA protein in culture medium conditioned by the indicated cells, performed by the Microparticle Enzyme Immunoassay method using IMx operation system (Abbott Diagnostics, Abbott Park, Ill., USA) binding to DNA for repression (Kley et al., Nucleic Acids Res., 20:4083-4087 (1992); Sun et al., J. Biol. Chem., 274:11535-11540 (1999); Xu et al., Oncogene, 19:5123-5133 (2000).

FIG. 2: Opposite regulatory effect of p53 on PSA and p21 promoters. Chloramphenicol acetyltransferase (CAT)-reporter constructs were used to estimate p53 influence on PSA and p21 promoter elements. A panel of reporter constructs: pBasic-CAT (CAT gene under minimal thymidine kinase promoter), pWAF1-CAT (p53-binding site from p21/Waf1 gene upstream of the minimal thymidine kinase promoter); p407ECAT plasmid, containing 1.6 kb enhancer (−5322 to −3740) and 418 bp promoter (−407 to +11) elements from PSA gene followed by promoterless CAT (Zhang et al., Biochem. Biophys. Res. Comm., 231:784-788 (1997); Zhang et al., Nucleic Acids Res., 25:3143-3150 (1997) were transfected into LNCaP cells in combination with different amounts of pLp53SN, containing human wild type p53, pLGSE56SN expressing GSE56 or empty pLXSN vector using Lipofectamin Plus reagent (Gibco BRL). Bars reflect relative CAT activity in lysates of LNCaP cells transiently transfected with either PSA-CAT (upper panel) or p21-CAT (lower panel) constructs in combination with the indicated plasmids. Results are normalized according to transfection efficiency and CAT expression in control cells transfected with insert-free vector. wt, plasmid expressing wild type human p53 cDNA; GSE, plasmid. (1) and (2) indicate plasmid concentration in micrograms. The experiment was repeated three times and showed similar results with variations in relative CAT activity values less than 20 percent.

FIG. 3: Trichostatin A (TSA) treatment eliminates the effect p53 has on PSA promoter activity. Bars show relative CAT activity in lysates of LNCaP cells transiently transfected with the indicated plasmid DNAs. TSA (100 nM) was added 5 h and CAT activity was measured 40 h post-transfection. Values reflect average of three independent experiments normalized according to transfection efficiency and CAT expression in control cells transfected with insert-free vector with no TSA. 156Pro mutants had no detectable effect on PSA. Thus, the dominant negative activity of tumor-derived p53 mutants is well correlated with the increased production of PSA by LNCaP cells, suggesting that similar events occur during tumor progression.

FIG. 4: Effect of tumor-derived p53 mutants on the levels of p21 protein expression and on PSA secretion by LNCaP cells. LNCaP cells were transduced with insert-free retrovirus or retroviruses expressing indicated p53 mutants. A panel of constructs expressing p53 mutants (pPS-p53^{135val}, pPS-p53^{141Ala}, pPS-p53^{156Pro}, pPS-p53^{175His}) was prepared in Mo- MuLV-based retroviral vector pPS-Hygro, expressing the p53 cDNA under the control of LTR and the hygromycin resistance gene under the control of SV40 promoter (Ossovskaya et al., Proc. Natl. Acad. Sci. USA, 93:10309-10314 (1996). Expression of p53 and p21 proteins was detected in the lysates of untreated LNCaP cell populations by Western immunoblotting with appropriate antibodies. Before loading, samples were normalized according to protein amounts confirmed by membrane staining and probing with anti-actin antibodies. 24-hour medium was collected from the same cell cultures and amounts of PSA protein were measured by Microparticle Enzyme Immunoassay, using IMx operation system (Abbott Diagnostics, Abbott Park, Ill., USA).

FIG. 5 contains the sequences of the genes listed in Tables 5 and 6, in order of the sequence ID number (SEQ ID NO). Note that the GenBank accession Number of the mouse EST printed on the chip is given in the tables; the name of the corresponding human consensus sequence [mRNA] (obtained by bioinformatic analysis) and the GenBank ID(s) of the sequence closest to the consensus sequence, where available, was added into Tables 5 and 6. FIG. 5 contains the human consensus sequence of each gene, where available, or the mouse consensus sequence, if the human sequence was unavailable, or the mouse EST sequence, if neither human nor mouse consensus sequence was available. The sequence identifier (SEQ ID NO), and corresponding Genbank accession number, are denoted before each sequence.

This application contains six Excel tables (Table 1-6 discussed herein). These tables are attached to this application as printed tables and also on a diskette.

### DETAILED DESCRIPTION OF THE PREFERRED EMBODIMENTS

The invention deals with methods of obtaining genetic markers for diagnosis and prognosis of cancer and methods for the use of these markers.

A preferred embodiment of the diagnostic aspect concerns a method of diagnosing a cancer in a subject comprising determining, in a sample from the subject, the level of at least one polypeptide, wherein a higher level of the polypeptide compared to the level of the polypeptide in a subject free of cancer is indicative of cancer, and wherein the polypeptide is selected from the group consisting of: polypeptides encoded by the human polynucleotides or the human orthologs of mouse polynucleotides listed in Table 5 or 6, and homologs of said polypeptides having at least 70% homology, preferably at least 80% homology, more preferably at least 90% homology.

The sample may be taken from a bodily fluid, such as blood, lymph fluid, ascites, serous fluid, pleural effusion, sputum, cerebrospinal fluid, lacrimal fluid, synovial fluid, saliva, stool, sperm and urine. The sample may also originate from a tissue, such as brain, lung, liver, spleen, kidney, pancreas, intestine, colon, mammary gland or breast, stomach, prostate, bladder, placenta, uterus, ovary, endometrium, testicle, lymph node, skin, head or neck, esophagus, bone marrow, and blood or blood cells.

General protocols for the detection of cancer markers can be found in “Tumor Marker Protocols”, Hanausek & Walaszek (Eds.), Humana Press, 1998. Methods of determining the level of a polypeptide in a sample are well known in the art (see, for example: Coligan et al, Unit 9, Current Protocols in Immunology, Wiley Interscience, 1994) and include, inter alia: immunohistochemistry (Microscopy, Immunohistochemistry and Antigen Retrieval Methods: For Light and Electron Microscopy, M. A. Hayat (Author), Kluwer Academic Publishers, 2002; Brown C.: “Antigen retrieval methods for immunohistochemistry”, Toxicol Pathol 1998; 26(6): 830-1; ELISA (Onorato et al., “Immunohistochemical and ELISA assays for biomarkers of oxidative stress in aging and disease”, Ann NY Acad Sci 1998 20; 854: 277-90), western blotting (Laemmeli UK: “Cleavage of structural proteins during the assembley of the head of a bacteriophage T4”, Nature 1970;227: 680-685; Egger & Bienz, “Protein (western) blotting”, Mol Biotechnol 1994; 1(3): 289-305), antibody microarray hybridization (Huang, “detection of multiple proteins in an antibody-based protein microarray system, Immunol Methods 2001 1; 255 (1-2): 1-13) and Targeted molecular imaging, which can be carried out on the whole body with imaging agents such as antibodies against the marker polypeptides (which may be membrane-bound proteins), the marker polypeptides themselves, receptors and contrast agents. The visualizations techniques include single photon and positron emission tomography, magnetic resonance imaging (MRI), computed tomography or ultrasonography (Thomas, Targeted Molecular Imaging in Oncology, Kim et al (Eds)., Springer Verlag, 2001). Any other known methods of polypeptide detection are also envisaged in connection with the invention. Optimization of protein detection procedures for diagnosis is well known in the art and described herein below. Specifically, diagnostic assays using the above methods may be carried out essentially as follows: Immunohistochemistry for diagnosis may be carried out essentially as described in Diagnostic Immunohistochemistry, David J., MD Dabbs, Churchill Livingstone, 1^{st} Ed, 2002; Quantitative Immunohistochemistry: Theoretical Background and its Application in Biology and Surgical Pathology, Fritz et al., Gustav Fischer, 1992. Western blotting-based diagnosis may be carried out essentially as described in Brys et al., “p53 protein detection by the Western blotting technique in normal and neoplastic specimens of human endometrium”, Cancer Letters 2000; 148 (197-205); Rochon et al., “Western blot assay for prostate-specific membrane antigen in serum of prostate cancer patients” Prostate 1994; 25(4): 219-23; Dalmau et al., “Detection of the anti-Hu antibody in the serum of patients with small cell lung cancer—a quantitative western blot analysis”, Ann Neurol 1990; 27(5): 544-52; Joyce et al., “Detection of altered H-ras proteins in human tumors using western blot analysis”, Lab Invest 1989; 61(2): 212-8. ELISA based diagnosis may be carried out essentially as described in D'ambrosio et al., “An enzyme-linked immunosorbent assay (ELISA) for the detection and quantitation of the tumor marker 1-methylinosine in human urine”, Clin Chim Acta 1991; 199(2): 119-28; Attalah et al., “A dipstick, dot-ELISA assay for the rapid and early detection of bladder cancer”, Cancer Detect Prev 1991; 15(6): 495-9; Erdile et al., “Whole cell ELISA for detection of tumor antigen expression in tumor samples”, Journal of Immunological Methods 2001; 258: 47-53. Antibody microarray-based diagnosis may be carried out essentially as described in Huang, “detection of multiple proteins in an antibody-based protein microarray system, Immunol Methods 2001 1; 255 (1-2): 1-13. Targeted molecular imaging-based diagnosis may be carried out essentially as described in Thomas, Targeted Molecular Imaging in Oncology, Kim et al (Eds)., Springer Verlag, 2001; Shahbazi-Gahrouei et al., “In vitro studies of gadolinium-DTPA conjugated with monoclonal antibodies as cancer-specific magnetic resonanace imaging contrast agents”, Australas Phys Eng Sci Med 2002; 25(1): 31-8; Tiefenauer et al., “Antibody-magnetite nanoparticles: in vitro characterization of a potential tumor-specific contrast agent for magnetic resonance imaging”, Bioconjug Chem 1993; 4(5): 347-52; Cerdan et al., “Monoclonal antibody-coated magnetite particles as contrast asents in magnetic resonance imaging of tumors”, Magn Reson Med 1989; 12(2): 151-63. In addition, polypeptides may be detected and a diagnostic assay performed using Mass Spectrometry, essentially as described in Bergquist et al., “peptide mapping of proteins in human body fluids using electrospray ionization fourier transform ion cyclotron resonance mass spectrometry”, Mass Spectrometry Reviews, 2002; 21:2-15 and Gelpi, “Biomedical and biochemical applications of liquid-chromatography-mass spectrometry”, Journal of Chromatography A, 1995; 703: 59-80.

An additional embodiment of the diagnostic aspect of the invention provides for a method of diagnosing a cancer in a subject comprising determining, in a sample from the subject, the level of at least one polypeptide-encoding polynucleotide, wherein a higher level of the polynucleotide compared to the level of the polynucleotide in a subject free of cancer is indicative of cancer, and wherein the polynucleotide is selected from the group consisting of human polynucleotides or the human orthologs of mouse polynucleotides listed in Tables 5 and 6, preferably in Table 6, polynucleotides having sequences that differ from these polynucleotides without changing the polypeptide encoded thereby, and homologs thereof having at least 70% homology, preferably at least 80% homology, more preferably at least 90% homology.

The sample may originate from a tissue or a bodily fluid, as described above.

Methods of determining the level of a polynucleotide in a sample are well known in the art and include, inter alia: RT-PCR analysis, in-situ hybridization and northern blotting; polynucleotide detection may also be performed by hybridizing a sample with a microarray imprinted with markers. Any other known methods of polynucleotide detection are also envisaged in connection with the invention. Optimization of polynucleotide detection procedures for diagnosis is well known in the art and described herein below. Specifically, diagnostic assays using the above methods are well known in the art (see, for example: Sidransky, “Nucleic Acid-Based methods for the Detection of Cancer”, Science, 1997; 278: 1054-1058) and may be carried out essentially as follows: RT-PCR for diagnosis may be carried out essentially as described in Bernard & Wittwer, “Real-Time PCR Technology for Cancer Diagnostics”, Clinical Chemistry 2002; 48(8): 1178-85; Raj et al., “Utilization of Polymerase Chain Reaction Technology in the Detection of Solid Tumors”, Cancer 1998; 82(8): 1419-1442; Zippelius & Pantel, “RT-PCR-based detection of occult disseminated tumor cells in peripheral blood and bone marrow of patients with solid tumors. An overview”, Ann NY Acad Sci 2000; 906:110-23. In-situ hybridization for diagnosis may be carried out essentially as described in “Introduction to Fluorescence In Situ Hybridization: Principles and Clinical Applications”, Andreeff & Pinkel (Editors), John Wiley & Sons Inc., 1999; Cheung et al., “Interphase cytogenetic study of endometrial sarcoma by chromosome in situ hybridization, modern Pathology 1996; 9:910-918. Northern blotting for diagnosis may be carried out essentially as described in Trayhurn, “Northern blotting”, Proc Nutr Soc 1996; 55(1B): 583-9; Shifman & Stein, “A reliable and sensitive method for non-radioactive Northern blot analysis of nerve growth factor mRNA from brain tissues”, Journal of Neuroscience Methods 1995; 59: 205-208; Pacheco et al., “Prognostic significance of the combined expression of matrix metalloproteinase-9, urokinase type plasminogen activator and its receptor in breast cancer as measured by Northern blot analysis”, Int J Biol Markers 2001; 16(1): 62-8. Polynucleotide microarray-based diagnosis can be carried out essentially as described in Ring & Boss, “Microarrays and molecular markers for tumor classification”, Genome Biol 2002; 3(5): comment2005; Lacroix et al., “A low-density DNA microarray for analysis of markers in breast cancer”, Int J Biol Markers 2002; 17(l): 5-23. In addition, polynucleotide microarray hybridization for diagnosis may be carried out essentially as described in the following review concerning micorarrays in the diagnosis of various cancers: Schmidt & Begley, “Cancer diagnosis and microarrays”, The International Journal of Biochemistry and Cell Biology, 2003; 35: 119-124. Diagnostic assays using tissue microarrays are also possible and may be performed essentially as described in Ginestier et al., “Distinct and comlementary information provided by use of tissue and DNA microarrays in the study of breast tumor markers”, Am J Pathol 2002; 161(4): 1223-33; Fejzo & Slamon, “Frozen tumor tissue microarray technology for analysis of tumor RNA, DNA and proteins”, Am J Pathol 2001; 159(5): 1645-50.

An example of detection of polynucleotides in bodily fluid is that of “staging” markers, which determine the stage of a cancer by detection of the presence of specific cancer cells in the blood (micrometastases) by RT-PCR of identified cancer-type-specific markers expression on the whole blood RNA (provided these markers are not normally expressed in blood cells) such detection and diagnosis can be carried out essentially as described in Luke & Kaul, “Detection of Breast Cancer Cells in Blood Using Immunomagmetic Bead Selection and Reverse Transcription-Polymerase Chain Reaction”, Mol Diagn 1998; 3(3): 149-155; Ghossein et al., “Molecular Detection of Micrometastases and Circulating Timor Cells in Solid Tumors”, Clinical Cancer Research 1999; 5: 1950-1960; Mellado et al., “Detection of circulating neoplastic cells by reverse-transcripatse polymerase chain reaction in malignant melanoma: association with clinical stages and prognosis”, J Clin Oncol 1996; 14(7): 2091-7.

Any of the diagnostic methods as described above can also be used together, simultaneously or not, and can thus provide a stronger diagnostic tool and validate or strengthen the results of a particular diagnosis. For combinations of different diagnostic methods see, inter alia: Hoshi et al., Enzyme-linked immunosorbent assay detection of prostate-specific antigen messenger ribonucleic acid in prostate cancer”, Urology 1999; 53 (1): 228-235; Zhong-Ping et al., “Quantitation of ERCC-2 Gene Expression in Human Tumor Cell Lines by Reverse Transcription-Polymerase Chain Reaction in Comparison to Northern Blot Analysis”, Analytical Biochemistry 1997; 244: 50-54; Hatta et al., “Polymerase chain reaction and immunohistochemistry frequently detect occult melanoma cells in regional lymph nodes of melanoma pateints”, J Clin Pathol 1998; 51(8): 597-601.

Any one of the diagnostic methods of the invention as recited above may also be employed to examine the status of a tumor suppressor gene or a biological pathway in which a tumor suppressor gene is involved, or to examine the effectiveness of a modulator of the activity of a tumor suppressor gene, such as a drug. The tumor suppressor gene in question may preferably be any one of p53, Rb1 and PTEN, as well as any other tumor suppressor gene deemed suitable. A list of tumor suppressor genes is provided above.

A preferred embodiment of the prognostic aspect of the invention concerns a method of measuring the responsiveness of a subject to a cancer treatment comprising determining the level of at least one polypeptide in a sample taken from the subject before treatment, and comparing it with the level of said polypeptide in a sample taken from the subject after treatment, a decrease in said level indicating responsiveness of said subject to the cancer treatment, wherein the polypeptide is selected from the group consisting of: polypeptides encoded by the human polynucleotides or the human orthologs of mouse polynucleotides listed in Table 5 or 6, and homologs of said polypeptides having at least 70% homology, preferably at least 80% homology, more preferably at least 90% homology.

As mentioned herein, the sample may be taken from a bodily fluid, as described above; the level of the polypeptide in the sample can be determined as described above.

In addition, the prognostic aspect of the invention comprises further a method of measuring the responsiveness of a subject to a cancer treatment comprising determining the level of at least one polypeptide-encoding polynucleotide in a sample taken from the subject before treatment, and comparing it with the level of said polynucleotide in a sample taken from the subject after treatment, a decrease in said level indicating responsiveness of said subject to the cancer treatment, wherein the polynucleotide is selected from the group consisting of: human polynucleotides or the human orthologs of mouse polynucleotides listed in Table 5 and 6, preferably in Table 6, polynucleotides having sequences that differ from these polynucleotides without changing the polypeptide encoded thereby, and homologs thereof having at least 70% homology, preferably at least 80% hormology, more preferably at least 90% homology.

The sample may originate from a tissue, preferably blood or bone marrow cells, or a bodily fluid, as described above.

The level of the polynucleotide in the sample is determined by the methods disclosed above, preferably by RT-PCR analysis. Any other polynucleotide detection methods disclosed herein may also be employed.

In accordance with the prognostic aspect of the invention, the treatment in conjunction with which the above methods of measuring the responsiveness of a subject to a cancer treatment may be employed include, inter alia, radiotherapy or administration of a chemotherapeutic drug such as etoposide, 5-FU (5-fluorouracil), cis-platinum, doxorubicin, a vinca alkaloid, vincristine, vinblastine, vinorelbine, taxol, cyclophosphamide, ifosfamide, chlorambucil, busulfan, mechlorethamine, mitomycin, dacarbazine, carboplatinum, thiotepa, daunorubicin, idarubicin, mitoxantrone, bleomycin, esperamicin A1, dactinomycin, plicamycin, carmustine, lomustine, tauromustine, streptozocin, melphalan, dactinomycin, procarbazine, dexamethasone, prednisone, 2-chlorodeoxyadenosine, cytarabine, docetaxel, fludarabine, gemcitabine, herceptin, hydroxyurea, irinotecan, methotrexate, oxaliplatin, rituxin, semustine, tomudex and topotecan, and chemotherapeutically active analogs of these drugs.

In a further embodiment of the prognostic aspect of the invention, the methods disclosed herein may also be indicative of the status of a tumor suppressor gene, as described above. Where a tumor suppressor gene or a pathway in which such gene is involved is defective or abnormal, this information may also serve in prognosis of both disease progression and treatment responsiveness of a patient, regardless of whether said treatment is directed to the tumor suppressor in question.

In an additional embodiment, the diagnostic and prognostic methods of the invention may also be carried out essentially as described herein wherein the method comprises determining the level of at least two polypeptides or polypeptide-encoding polynucleotides in a sample taken from a subject. Methods of determining the level of polypeptides and polynucleotides are described above.

Different combinations of polypeptides or polynucleotides of the cancer markers may be employed in different diagnostic or prognostic methods for various cancers.

For bodily fluid sample based diagnosis or prognosis, at least one polypeptide or combination of at least two polypeptides encoded by the human polynucleotide or human orthologs of the polynucleotides, of Table 3 and 5, preferably of Table 5, more preferably of the highlighted genes of Table 5, may be employed as markers.

For tissue sample based diagnosis or prognosis at least one polypeptide or combination of at least two polypeptides encoded by the human polynucleotide or human orthologs of the polynucleotides, of Table 2 and 6, preferably of Table 6, or the polynucleotides themselves may be employed as markers.

For the diagnosis or prognosis of a cancer of a specific tissue, the markers comprise at least one, preferably at least 2, human polypeptides or polynucleotides, or human orthologs of the mouse polypeptides or polynucleotides, or homologs thereof, listed in Table 2 and Table 6. For the tissues breast, placenta/uterus, kidney, bladder, lung, brain, colon, intestine, stomach, liver, pancreas and spleen the above described polypeptides and polynucleotides are listed in Table 2 and Table 6 as follows:

For the diagnosis or prognosis of a cancer of the breast, the markers listed in Table 2 sheet 1 and Table 6, preferably in Table 6 under the heading “breast”;

For the diagnosis or prognosis of a cancer of the uterus, the markers listed in Table 2 sheet 2 and Table 6, preferably in Table 6 under the heading “placenta/uterus”;

For the diagnosis or prognosis of a cancer of the kidney, the markers listed in Table 2 sheet 3 and Table 6, preferably in Table 6 under the heading “kidney”;

For the diagnosis or prognosis of a cancer of the bladder, the markers listed in Table 2 sheet 4 and Table 6, preferably in Table 6 under the heading “bladder”;

For the diagnosis or prognosis of a cancer of the lung, the markers listed in Table 2 sheet 5 and Table 6, preferably in Table 6 under the heading “lung”;

For the diagnosis or prognosis of a cancer of the brain, the markers listed in Table 2 sheet 6 and Table 6, preferably in Table 6 under the heading “brain”;

For the diagnosis or prognosis of a cancer of the colon, the markers listed in Table 2 sheet 7 and Table 6, preferably in Table 6 under the heading “colon”;

For the diagnosis or prognosis of a cancer of the intestine, the markers listed in Table 2 sheet 8 and Table 6, preferably in Table 6 under the heading “intestine”;

For the diagnosis or prognosis of a cancer of the stomach, the markers listed in Table 2 sheet 9 and Table 6, preferably in Table 6 under the heading “stomach”;

For the diagnosis or prognosis of a cancer of the liver, the markers listed in Table 2 sheet 10 and Table 6, preferably in Table 6 under the heading “liver”;

For the diagnosis or prognosis of a cancer of the pancreas, the markers listed in Table 2 sheet 11 and Table 6, preferably in Table 6 under the heading “pancreas”;

For the diagnosis or prognosis of a cancer of the spleen, the markers listed in Table 2 sheet 12 and Table 6, preferably in Table 6 under the heading “spleen.”

The invention further comprises a method of identifying a diagnostic marker for a cancer comprising:

(a) obtaining a first cell from a first cell type of said cancer, said cell comprising a defective tumor suppressor expression;

(b) obtaining a second cell of the first cell type, wherein said second cell comprises a wild-type tumor suppressor expression;

(c) identifying genes having an increased level of expression in the first cell as compared to the second cell; and

(d) selecting at least one gene of step (c) as a diagnostic marker for a cancer.

In a related aspect, the invention further comprises a method of identifying a tissue-specific diagnostic marker for a cancer comprising a) obtaining a first cell from a second cell type of the cancer, the cell comprising a defective tumor suppressor expression; b) obtaining a second cell of the second cell type, wherein the second cell comprises a wild-type tumor suppressor expression; c) identifying genes having an increased level of expression in the first cell of the second cell type as compared to the second cell of the second cell type; d) comparing the genes having an increased expression in the first cell type with the genes having an increased expression in the second cell type; e) identifying genes having an increased expression in the first cell type but not in the second cell type; and f) selecting at least one gene of step (e) as a diagnostic marker of a cancer of the first cell type.

The identification step of both methods (steps (c) or e) above, respectively) may be performed using a microarray; in addition, the tumor suppressor in question may be p53, Rb1 and PTEN as well as any other tumor suppressor gene deemed suitable. A list of possible tumor suppressor genes is provided herein.

In certain embodiments, the diagnostic marker is a secreted product of the first cell type. In certain embodiments, the selected gene is not expressed in other tissue irrespective of its status. In other embodiments, the diagnostic marker is a membrane bound marker that localizes to the cell membrane of the first cell type. In specific embodiments, the tumor suppressor is selected from the group consisting of p53, Rb1, APC; BRCA1; BRCA2; CDH1; p57, p16, CYLD; p300; EXT1; EXT2; MADH4; MAP2K4; MEN1; HNPCC2; MSH2; NF1; NF2; PRKAR1A; PTCH; PTEN; SDHD; SMARCB1; STK11; TSC1; TSC2; VHL and WT1.

An additional embodiment of the invention concerns a method for screening for compounds that modulate the activity of a tumor suppressor gene comprising: a) obtaining a cell comprising a defective tumor suppressor expression; b) measuring the level of expression of a marker of Table 5 or 6 in the cell; c) contacting the cell with a test compound; and d) measuring the expression of the marker of step b) after the contacting step c), wherein a change in the level of expression after the contacting step as compared to the level of expression before the contacting step is indicative of the ability of the compound to modulate the activity of the tumor suppressor gene.

The tumor suppressor in question may be selected from the tumor suppressor group consisting of, inter alia, p53, Rb1, APC; BRCA1; BRCA2; CDH1; p57, p16, CYLD; p300; EXT1; EXT2; MADH4; MAP2K4; MEN1; HNPCC2; MSH2; NF1; NF2; PRKAR1A; PTCH; PTEN; SDHD; SMARCB1; STK11; TSC1; TSC2; VHL and WT1. The test compound may be a small chemical molecule. The measuring of steps b) and d) may comprise monitoring the level of mRNA of the marker or the level of the polypeptide of the marker, according to methods well known in the art and described herein. In addition, the change in the level of expression in step d) may be a reduction in the level of expression, in which case compounds identified according to said method may be employed in the treatment of cancer, possibly as anti-cancer drugs.

The term “small chemical molecule” is used interchangeably with “chemical compound”, and is understood to refer to chemical moieties of any particular type which are not necessarily, but may be, naturally occurring and typically have a molecular weight of less than 2000 daltons, more preferably less than 1000 daltons.

Another aspect of the invention provides a microarray composition for measuring tissue-specific gene expression comprising at least 4 polynucleotides from tables 5 and 6. The invention further contemplates a method of diagnosing a cancer comprising contacting a cell sample nucleic acid with a microarray described herein under conditions suitable for hybridization; providing hybridization conditions suitable for hybrid formation between said cell sample nucleic acid and a polynucleotide of said microarray; detecting said hybridization; and diagnosing a cancer based on the results of detecting said hybridization.

Further in this aspect, an antibody microarray is provided. Said microarray comprises at least 4 antibodies directed against polypeptides corresponding to the polynucleotides given in Tables 5 and 6. The invention further contemplates a method of diagnosing a cancer comprising contacting a bodily fluid sample with the antibody microarray described herein, and detecting hybridization between the antibodies present on the array and at least one polypeptide present in the bodily fluid, the results of said detection enabling a diagnosis or a prognosis of a cancer.

The invention further contemplates a vector comprising a polynucleotide having a sequence of a tissue specific tumor marker identified according to the invention. Also contemplated is a cell transformed or transfected with such a vector.

Another aspect of the invention is directed to a method of treating cancer in a patient, wherein said treatment is effected through the decrease in expression of a tumor marker gene. In preferred embodiments, a polynucleotide is administered to cancer cells of a patient. The polynucleotide comprises an antisense sequence of said tissue-specific tumor marker in those embodiments where the tissue-specific tumor marker is up-regulated as a result of loss of function of the tumor suppressor, whereas the polynucleotide comprises a sense coding sequence of said tissue-specific tumor marker in those embodiments where the tissue specific marker is down-regulated as a result of loss of function of the tumor suppressor. In specific embodiments, the cancer cells of the patient harbor a mutant tumor suppressor gene selected from the group consisting of p53, Rb1, APC; BRCA1; BRCA2; CDH1; p57, p16, CYLD; p300; EXT1; EXT2; MADH4; MAP2K4; MEN1; HNPCC2; MSH2; NF1; NF2; PRKAR1A; PTCH; PTEN; SDHD; SMARCB1; STK11; TSC1; TSC2; VHL and WT1.

By “homolog/homology”, as related to polynucleotides and polypeptides and used herein, is meant at least about 70%, preferably at least about 75% homology, advantageously at least about 80% homology, more advantageously at least about 90% homology, even more advantageously at least about 95%, e.g., at least about 97%, about 98%, about 99% or even about 100% homology. The invention also comprehends that these polynucleotides and polypeptides can be used in the same fashion as the herein or aforementioned polynucleotides and polypeptides.

Alternatively or additionally, “homology”, with respect to sequences, can refer to the number of positions with identical nucleotides or amino acid residues, divided by the number of nucleotides or amino acid residues in the shorter of the two sequences, wherein alignment of the two sequences can be determined in accordance with the Wilbur and Lipman algorithm ((1983) Proc. Natl. Acad. Sci. USA 80:726), for instance, using a window size of 20 nucleotides, a word length of 4 nucleotides, and a gap penalty of 4, and computer-assisted analysis and interpretation of the sequence data, including alignment can be conveniently performed using commercially available programs (e.g., Intelligenetics™ Suite, Intelligenetics Inc., CA). When RNA sequences are said to be similar, or to have a degree of sequence identity or homology with DNA sequences, thymidine (T) in the DNA sequence is considered equal to uracil (U) in the RNA sequence. RNA sequences within the scope of the invention can be derived from DNA sequences or their complements, by substituting thymidine (T) in the DNA sequence with uracil (U).

Additionally or alternatively, amino acid sequence similarity or homology can be determined, for instance, using the BlastP program (Altschul et al., Nucl. Acids Res. 25:3389-3402) and available at NCBI. The following references provide algorithms for comparing the relative identity or homology of amino acid residues of two polypeptides, and additionally, or alternatively, with respect to the foregoing, the teachings in these references can be used for determining percent homology: Smith et al., (1981) Adv. Appl. Math. 2:482-489; Smith et al., (1983) Nucl. Acids Res. 11:2205-2220; Devereux et al., (1984) Nucl. Acids Res. 12:387-395; Feng et al., (1987) J. Molec. Evol. 25:351-360; Higgins et al., (1989) CABIOS 5:151-153; and Thompson et al., (1994) Nucl. Acids Res. 22:4673-4680.

The term “polynucleotide” refers to any molecule which comprises two or more of the bases guanidine, citosine, timidine, adenine, uracil or inosine, inter alia, or chemical analogs thereof, includes “oligonucleotides” and encompasses “nucleic acids”. Preferably, a polynucleotide has from about 75 to 10,000 nucleotides, more preferably from about 100 to 3,500 nucleotides. An oligonucleotide refers generally to a chain of nucleotides extending from 2-75 nucleotides.

By the term “polypeptide” is meant a molecule composed of amino acids and the term includes peptides, polypeptides, proteins and peptidomimetics; dominant polypeptide fragments are also considered to be polypeptides.

The term “amino acid” refers to any one of the 20 naturally occurring amino acids, and also amino acids which have been chemically modified or synthetic amino acids.

The invention provides methods for the identification of marker gene targets for both diagnostic and therapeutic applications in any given cancer type. In certain embodiments, these methods use a combination of recently developed powerful functional gene cloning methodologies with cDNA array-based gene expression profiling and rationally designed experimental models. Diagnostic and therapeutic value of the identified genes may then be evaluated using specific inhibitors and antibodies according to methods well known to those of skill in the art.

By identifying those genes that are specifically upregulated (or indeed downregulated) in cancer cells as a result of tumor suppressor regulation, the invention provides markers of advanced stages of cancer. More specifically, the invention relates to identifying potential targets of tumor suppressor regulation associated with early and advanced stages of the disease by performing micro-array hybridization and analyses using model cancer cell line(s) or primary normal cell cultures that retain wild-type tumor suppressor activity and engineering a variant of such a cell line or primary cells in which the tumor suppressor is inactivated. Alternatively, the tissue pairs for comparison will be normal animal tissues and the same cancer-free tissues from genetically modified animals in which a tumor suppressor gene of interest was knocked out.

The methods of the invention generally provide a systematic approach for the search of cancer markers or targets for therapeutic intervention among the genes normally under negative control of tumor suppressor proteins. Many such genes are transcriptionally activated in tissues following the wild-type activity loss of the most common tumor suppressor genes, such as p53, PTEN, RB, and p16/p19 and this regulation is conserved in normal and tumor cells from the same origin. The methods of the invention may be performed by comparing gene expression profiles in the isogenic pairs of cell lines or tissues differing in their tumor suppressor gene status or tissue pairs derived from normal and genetically modified mice, with inactivated tumor suppressors, i.e. p53-/- mice, p16/p19-/- mice, and mice with targeted expression of, e.g., SV40 large T antigen that simultaneously inactivates both RB and p53 function (TRAMP mice).

In an exemplary model for the invention, the inventors created an isogenic pair of LNCaP prostate tumor cell lines differing in their p53 status and applied cDNA microarray analysis to identify differentially expressed genes. These investigations revealed that the baseline expression of several known tumor markers is significantly elevated in LNCaP cells that lack functional p53 protein compared to the same cells that express wt p53. These genes include e.g., COX2, tumor-specific heparin-binding growth factor midkine (which possesses angiogenic and anti-apoptotic properties) (Ikematsu et al., Br J Cancer, 83(6):701-706 (2000), tumor tissue associated hyaluronan receptor CD44 and PSA (prostate specific antigen). COX2 inhibitors are currently in clinical trials against prostate cancer. Midkine was immunohistochemically shown to be expressed in 86.3% of prostate cancer specimens examined, with metastatic lesions generally showing higher expression than the corresponding primaries; normal prostate tissues were negative or showed only weak staining. Midkine was also detected in 12 of 15 latent cancers (80%) and in 12 of 16 cases of PIN (75%) (Konishi et al., Oncology; 57(3):253-257 (1999).

PSA is the major prostate cancer diagnostic marker currently used commercially. In the invention, it was shown that the PSA promoter is directly suppressed by wt p53, thus PSA up-regulation in prostate cancer is indicative of the loss of wt p53 function. The list of genes the expression of which was changed following wt p53 suppression in LNCaP cells is attached in Table 1. Having determined that it is thus possible to identify the differential expression of genes that are regulated by suppression of the wild-type tumor suppressor, the inventors further demonstrate large-scale microarray-based comparison of gene-expression profiles in the tissue pairs derived from normal and p53-/- mice.

Poly A RNA was extracted from spleen, pancreas, liver, stomach, intestine, colon, lung, brain, bladder, kidney, placenta/uterus and mammary glands of normal and p53-deficient mice and used for fluorescently-labeled probes for microarray hybridizations. The differential (against common control) gene expression levels were normalized between p53-/- tissues and their corresponding normal counterparts. (Table 2). These data were then sorted according to their expression levels in one particular tissue from maximally up-regulated genes to maximally down-regulated genes, thereby identifying genes with maximal differential tissue-specific expression in p53-deficient mice.

Of the identified genes, the tumor makers will be those that are found to be up-regulated in p53-/-tissues. Table 3 lists such genes; the table combines the p53-dependent differential expression data with the tissue specificity of gene expression data. Differential expression of the genes may be determined using any technique well known to those of skill in the art. Such techniques include determining differential expression using cDNA or oligonucleotide microarrays as described herein below, as well as differential display techniques well known to those of skill in the art. Gene subtraction techniques also may be used. Also contemplated for determining differential expression of genes is SAGE (Velculescu et al., Science, 270:484-487 (1995); Zhang et al., Science, 276:1268-1272 (1997).

For effective selection of cancer diagnostic markers, the following criteria were applied:

(1) genes that are up-regulated in a certain p53-/- tissue and are normally expressed predominantly in that tissue are useful for diagnosis both in tissues and in bodily fluids. Table 5 is derived from Table 3 and contains a list of the preferred genes which can serve as markers of this type (the highlighted genes are highly preferred).

(2) genes that are normally expressed at certain levels in one or several tissues but are up-regulated in one or numerous p53-/- tissues as compared to the same tissue having normal p53 status are useful for diagnosis primarily in tissues. Table 6 is derived from Table 2 and contains a list of the preferred markers of this type, sorted according to the tissue in which they are preferred for diagnosis. Both tables are prioritized, so that, for example, under the heading “pancreas” in Table 6 or in sheet 11 of Table 2, the first marker listed, pancreatitis associated protein, is the most preferred marker for pancreatic cancer.

Table 3 contains 445 genes identified as being up-regulated in p53-/- tissues, which can serve as tissue specific cancer markers and for bodily-fluid cancer diagnosis, depending on their level of expression in normal tissues, which tissues they are normally expressed in, and whether they are secreted.

The genes identified according to the invention will prove useful in diagnostic and prognostic application as well as act as drug targets for therapeutic intervention of the diseased state. Negative regulation by tumor suppressor genes and tissue specificity of expression are two essential characteristics of prospective tumor markers/drug targets. However, in order to be suitable for diagnostic assays, the gene products ideally, but not necessarily, also need to be secreted into blood, urine, saliva or any other accessible body fluids for detection. Alternatively, the gene products are such that they are expressed at the cell surface and are therefore amenable to detection using ordinary techniques known to those of skill in the art, e.g., detection of cell surface expression of the gene products using antibodies or ligand/receptor interactions. Membrane-bound and cytosolic RNA may be distinguished based on the fact that mRNA of genes, encoding secreted or membrane proteins is bound to membrane-associated polysomes and may be separated from other mRNAs by sedimentation equilibrium or sedimentation velocity (Diehn et al., Nat. Genet., 25:58-62, 2000). RNA from membrane or cytosolic fraction of cells will be isolated using standard protocol and used for synthesis of fluorescently labeled probe from each fraction. Isolation of membrane-bound polysomes from cell lines preferably is carried out according to published protocol (Diehn et al., Nat. Genet., 25:58-62 (2000). See also U.S. Pat. No. 6,403,316.

In summary, the inventors defined genes characterized in regard to tissue-specificity of the normal expression of these genes and induction/reduction in various p53-deficient tissues. The above-articulated method, while exemplified in terms of p53 regulation, may be performed with any tumor suppressor known to those of skill in the art to identify tissue-specific markers of cancers. In addition to p53, tumor suppressors such as Rb1, APC; BRCA1; BRCA2; CDH1; p57, p16, CYLD; p300; EXT1; EXT2; MADH4; MAP2K4; MEN1; HNPCC2; MSH2; NF1; NF2; PRKAR1A; PTCH; PTEN; SDHD; SMARCB1; STK11; TSC1; TSC2; VHL; WT1, are exemplary tumor suppressors that may be employed to identify tissue-specific tumor marker genes according to the invention. This is by no means an exhaustive list and those of skill in the art will be aware of other tumor suppressors that may be used in the methods herein. Those of skill in the art will readily be able to obtain the sequences for these tumor suppressor genes from Genbank.

I. Diagnostic Methods of Using Identified Markers

In the genetic diagnostic applications of the invention, one of skill in the art would detect variations in the expression of one or more of the tissue-specific tumor markers. This may comprise determining the mRNA level of the gene(s) or determining specific alterations in the expressed gene product(s). The cancers that may be diagnosed according to the invention include cancers of the brain (glioblastomas, medulloblastoma, astrocytoma, oligodendroglioma, ependymomas), lung, liver, spleen, kidney, pancreas, intestine, blood cells, lymph node, colon, breast, endometrium, stomach, prostate, testicle, ovary, skin, head or neck, esophagus, bone marrow, blood or other tissue.

The biological sample can be any tissue or fluid. Various embodiments include cells of the skin, muscle, facia, brain, prostate, breast, endometrium, lung, head or neck, pancreas, small intestine, blood cells, liver, testes, ovaries, colon, skin, stomach, esophagus, spleen, lymph node, bone marrow or kidney. Other embodiments include fluid samples such as peripheral blood, lymph fluid, ascites, serous fluid, pleural effusion, sputum, cerebrospinal fluid, lacrimal fluid, synovial fluid, saliva, stool or urine.

Nucleic acids can be isolated from cells contained in the biological sample, according to standard methodologies (Sambrook et al., 1989). The nucleic acid may be whole RNA. It may be used for Northern blotting analysis or may be converted to a complementary DNA (cDNA). In one embodiment, the RNA is whole cell RNA; in another, it is poly-A RNA. cDNA may be used for preparation of probes for microarray hybridization or may be amplified in PCR reaction (RT-PCR).

In situ hybridization using a labeled nucleic acid probe is performed essentially as known in the art and incorporated herein by reference.

Depending on the format, the specific nucleic acid of interest is identified in the sample directly using amplification or by hybridization to a labeled (radioactively or fluorescently) nucleic acid probe. Next the identified amplified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

A. Microarray Analyses

In certain preferred embodiments, DNA-based arrays provide a convenient way to explore the expression of a single polymorphic gene or a large number of genes for a variety of applications. The tissue-specific tumor marker nucleic acids identified by the invention may be presented in a DNA microarray for the analysis and expression of these genes in various cancer cell types. Microarray chips are well known to those of skill in the art (see, e.g., U.S. Pat. Nos. 6,308,170; 6,183,698; 6,306,643; 6,297,018; 6,287,850; 6,291,183, each incorporated herein by reference). These are exemplary patents that disclose nucleic acid microarrays and those of skill in the art are aware of numerous other methods and compositions for producing microarrays.

In addition, protein and antibody microarrays are well known in the art (see, for example: Ekins R. P., J Pharm Biomed Anal 1989. 7: 155; Ekins R. P. and Chu F. W., Clin Chem 1991. 37: 1955; Ekins R. P. and Chu F. W, Trends in Biotechnology, 1999, 17, 217-218). Antibody microarrays directed against a combination of the diagnostic markers disclosed herein will be very useful for the diagnosis of cancer markers in bodily fluids.

The invention provides for a composition comprising a plurality of polynucleotides identified according to the methods of the invention. As used herein, the term “polynucleotide probe” refers to any nucleic acid sequences identified according to the invention as a marker for a given cancer. Preferably, the polynucleotide fragment is at least 9 nucleotides; more preferably, it is at least 20 nucleotides. Such a composition can be employed for the diagnosis and treatment of neoplastic disorder.

The composition is particularly useful as hybridizable array elements in a microarray for monitoring the expression of a plurality of target polynucleotides. The microarray comprises a substrate and the hybridizable array elements. The microarray is used, for example, in the diagnosis and treatment of a cancer.

The term “microarray” refers to an ordered arrangement of hybridizable array elements. The array elements are arranged so that there are preferably at least two or more different array elements, more preferably at least 100 array elements, and most preferably at least 1,000 array elements, on a 1 cm² substrate surface. The hybridization signal from each of the array elements is individually distinguishable. In a preferred embodiment, the array elements comprise polynucleotide probes. In another preferred embodiment, the array elements comprise antibodies.

The term “probe” refers to a polynucleotide sequence capable of hybridizing with a target sequence to form a polynucleotide probe/target complex. A “target polynucleotide” refers to a chain of nucleotides to which a polynucleotide probe can hybridize by base pairing. In some instances, the sequences will be complementary (no mismatches) when aligned. In other instances, there may be up to a 10% mismatch.

Alternatively, the term “probe” may refer to a polypeptide probe that can hybridize to an antibody.

A “plurality” refers preferably to a group of at least 15 or more members, more preferably to a group of at least about 100, and even more preferably to a group of at least about 1,000, members. The maximum number of members is unlimited, but is at least about 100,000 members.

The term “gene” or “genes” refers to a polynucleotide sequence(s) of a gene, which may be the partial or complete sequence of the gene and may comprise regulatory region(s), untranslated region(s), or coding regions.

The polynucleotide or antibody microarray can be used for large-scale genetic or gene expression analysis of a large number of target polynucleotides or polypeptides respectively. The microarray can also be used in the diagnosis of diseases and in the monitoring of treatments. Further, the microarray can be employed to investigate an individual's predisposition to a disease. Furthermore, the microarray can be employed to investigate cellular responses to infection, drug treatment, and the like.

When the composition of the invention is employed as hybridizable array elements in a microarray, the array elements are organized in an ordered fashion so that each element is present at a distinguishable, and preferably specified, location on the substrate. In the preferred embodiments, because the array elements are at specified locations on the substrate, the hybridization patterns and intensities (which together create a unique expression profile) can be interpreted in terms of expression levels of particular genes and can be correlated with a particular disease or condition or treatment.

The composition comprising a plurality of polynucleotide probes can also be used to purify a subpopulation of mRNAs, cDNAs, genomic fragments and the like, in a sample. Typically, samples will include target polynucleotides of interest and other nucleic acids which may enhance the hybridization background; therefore, it may be advantageous to remove these nucleic acids from the sample. One method for removing the additional nucleic acids is by hybridizing the sample containing target polynucleotides with immobilized polynucleotide probes under hybridizing conditions. Those nucleic acids that do not hybridize to the polynucleotide probes are removed and may be subjected to analysis or discarded. At a later point, the immobilized target polynucleotide probes can be released in the form of purified target polynucleotides.

1. Microarray Production

The nucleic acid probes can be genomic DNA or cDNA or mRNA, or any RNA-like or DNA-like material, such as peptide nucleic acids, branched DNAs, and the like. The probes can be sense or antisense polynucleotide probes. Where target polynucleotides are double-stranded, the probes may be either sense or antisense strands. Where the target polynucleotides are single-stranded, the probes are complementary single strands.

In one embodiment, the probes are cDNAs. The size of the DNA sequence of interest may vary and is preferably from 100 to 10,000 nucleotides, more preferably from 150 to 3,500 nucleotides.

The probes can be prepared by a variety of synthetic or enzymatic schemes, which are well known in the art. The probes can be synthesized, in whole or in part, using chemical methods well known in the art (Caruthers et al., Nucleic Acids Res., Symp. Ser., 215-233 (1980). Alternatively, the probes can be generated, in whole or in part, enzymatically.

Nucleotide analogs can be incorporated into the probes by methods well known in the art. The only requirement is that the incorporated nucleotide analog must serve to base pair with target polynucleotide sequences. For example, certain guanine nucleotides can be substituted with hypoxanthine, which base pairs with cytosine residues. However, these base pairs are less stable than those between guanine and cytosine. Alternatively, adenine nucleotides can be substituted with 2,6-diaminopurine, which can form stronger base pairs than those between adenine and thymidine.

Additionally, the probes can include nucleotides that have been derivatized chemically or enzymatically. Typical chemical modifications include derivatization with acyl, alkyl, aryl or amino groups.

The polynucleotide probes can be immobilized on a substrate. Preferred substrates are any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the polynucleotide probes are bound. Preferably, the substrates are optically transparent.

Complementary DNA (cDNA) can be arranged and then immobilized on a substrate. The probes can be immobilized by covalent means such as by chemical bonding procedures or UV. In one such method, a cDNA is bound to a glass surface which has been modified to contain epoxide or aldehyde groups. In another case, a cDNA probe is placed on a polylysine coated surface and then UV cross-linked (Shalon et al., PCT publication WO95/35505, herein incorporated by reference). In yet another method, a DNA is actively transported from a solution to a given position on a substrate by electrical means (Heller et al., U.S. Pat. No. 5,605,662). Alternatively, individual DNA clones can be gridded on a filter. Cells are lysed, proteins and cellular components degraded, and the DNA coupled to the filter by UV cross-linking.

Furthermore, the probes do not have to be directly bound to the substrate, but rather can be bound to the substrate through a linker group. The linker groups are typically about 6 to 50 atoms long to provide exposure to the attached probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the probe.

The probes can be attached to a substrate by dispensing reagents for probe synthesis on the substrate surface or by dispensing preformed DNA fragments or clones on the substrate surface. Typical dispensers include a micropipette delivering solution to the substrate with a robotic system to control the position of the micropipette with respect to the substrate. There can be a multiplicity of dispensers so that reagents can be delivered to the reaction regions simultaneously.

Alternatively, as mentioned above, antibody microarrays can be produced. The production of such microarrays is essentially as described in Schweitzer & Kingsmore, “Measuring proteins on microarrays”, Curr Opin Biotechnol 2002; 13(1): 14-9; Avseenko et al., “Immobilization of proteins in immunochemical microarrays fabricated by electrospray deposition”, Anal Chem 2001 15; 73(24): 6047-52; Huang, “Detection of multiple proteins in an antibody-based protein microarray system, Immunol Methods 2001 1; 255 (1-2): 1-13. In general, protein microarrays may be produced essentially as described in Schena et al., Parallel human genome analysis: Microarray-based expression monitoring of 1000 genes. Proc. Natl. Sci. USA (1996) 93, 10614-10619; U.S. Pat. Nos. 6,291,170 and 5,807,522 (see above); U.S. Pat. No. 6,037,186 (Stimpson, inventor) “Parallel production of high density arrays”; PCT publications WO 99/13313 (Genovations Inc [US], applicant) “Method of making high density arrays”; WO 02/05945 (Max-Delbruck-center for molecular medicine [Germany], applicant) “Method for producing microarray chips with nucleic acids, proteins or other test substrates”.

2. Sample Preparation for Genetic Analysis

In order to conduct sample analysis, a sample containing target polynucleotides or polypeptides is provided. The samples can be any sample containing target polynucleotides or polypeptides and obtained from any bodily fluid (blood, sperm, urine, saliva, phlegm, gastric juices, etc. as described herein), cultured cells, biopsies, or other tissue preparations. The samples being analyzed using the microarrays will likely be samples from individuals suspected of suffering from a given cancer. In one embodiment, the microarrays used are those that contain tumor markers specific for that cancer or antibodies against those markers.

DNA or RNA can be isolated from the sample according to any of a number of methods well known to those of skill in the art. For example, methods of purification of nucleic acids are described in Tijssen Laboratory Techniques in Biochemistry and Molecular Biology: Hybridization With Nucleic Acid Probes, Part I. Theory and Nucleic Acid Preparation, Elsevier, New York N.Y. 1993. In one case, total RNA is isolated using the TRIZOL reagent (Life Technologies, Gaithersburg Md.), and mRNA is isolated using oligo d(T) column chromatography or glass beads. Alternatively, when target polynucleotides are derived from an mRNA, the target polynucleotides can be a cDNA reverse-transcribed from an mRNA, an RNA transcribed from that cDNA, a DNA amplified from that cDNA, an RNA transcribed from the amplified DNA, and the like. When the target polynucleotide is derived from DNA, the target polynucleotide can be DNA amplified from DNA or RNA reverse transcribed from DNA. In yet another alternative, the targets are target polynucleotides prepared by more than one method.

When target polynucleotides are amplified, it is desirable to amplify the nucleic acid sample and maintain the relative abundances of the original sample, including low abundance transcripts. Total mRNA can be amplified by reverse transcription using a reverse transcriptase and a primer consisting of oligo d(T) and a sequence encoding the phage T7 promoter to provide a single-stranded DNA template. The second DNA strand is polymerized using a DNA polymerase and a RNAse which assists in breaking up the DNA/RNA hybrid. After synthesis of the double-stranded DNA, T7 RNA polymerase can be added, and RNA transcribed from the second DNA strand template (Van Gelder et al. U.S. Pat. No. 5,545,522). RNA can be amplified in vitro, in situ or in vivo (See Eberwine, U.S. Pat. No. 5,514,545).

Quantitation controls may be included within the sample to assure that amplification and labeling procedures do not change the true distribution of target polynucleotides in a sample. For this purpose, a sample is spiked with a known amount, of a control target polynucleotide and the composition of probes includes reference probes which specifically hybridize with the control target polynucleotides. After hybridization and processing, the hybridization signals obtained should accurately the amounts of control target polynucleotide added to the sample.

Prior to hybridization, it may be desirable to fragment the nucleic acid target polynucleotides. Fragmentation improves hybridization by minimizing secondary structure and cross-hybridization to other nucleic acid target polynucleotides in the sample or noncomplementary polynucleotide probes. Fragmentation can be performed by mechanical or chemical means.

Antibodies against the relevant cancer marker polypeptides and appropriate for attachment to an antibody microarray can be prepared according to methods known in the art (Coligan et al, Unit 9, Current Protocols in Immunology, Wiley Interscience, 1994; Harlow and Lane, Antibodies: A Laboratory Manual, Cold Spring Harbor Laboratory, New York (1988). Additional information regarding all types of antibodies, including humanized antibodies, human antibodies and antibody fragments can be found in WO 01/05998).

Polypeptides can be prepared for hybridization to an antibody microarray from a sample, such as a bodily fluid sample, according to methods known in the art. It may be desirable to purify the proteins from the sample or alternatively, to remove certain impurities which may be present in the sample and interfere with hybridization. Protein purification is practiced as is known in the art as described in, for example, Marshak et al., “Strategies for Protein Purificationand Characterization. A laboratory course manual.” CSHL Press (1996).

The target polynucleotides or polypeptides may be labeled with one or more labeling moieties to allow for detection of hybridized probe/target complexes. The labeling moieties can include compositions that can be detected by spectroscopic, photochemical, biochemical, bioelectronic, immunochemical, electrical, optical or chemical means. The labeling moieties include radioisotopes, such as ³H, ¹⁴C, ³²P, ³³P or ³⁵S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

Exemplary dyes include quinoline dyes, triarylmethane dyes, phthaleins, azo dyes, cyanine dyes, and the like. Preferably, fluorescent markers absorb light above about 300 nm, preferably above 400 nm, and usually emit light at wavelengths at least greater than 10 nm above the wavelength of the light absorbed. Preferred fluorescent markers include fluorescein, phycoerythrin, rhodamine, lissamine, and C3 and C5 available from Amersham Pharmacia Biotech (Piscataway N.J.).

Nucleic acid labeling can be carried out during an amplification reaction, such as polymerase chain reactions and in vitro transcription reactions, or by nick translation or 5′ or 3′-end-labeling reactions. When the label may be incorporated after or without an amplification step, the label is incorporated by using terminal transferase or by phosphorylating the 5′ end of the target polynucleotide using, e.g., a kinase and then incubating overnight with a labeled oligonucleotide in the presence of T4 RNA ligase. Alternatively, the labeling moiety can be incorporated after hybridization once a probe/target complex has formed.

Polypeptide labeling can be conducted using a variety of techniques well known in the art, and the choice of the technique(s) can be tailored to the polypeptide in question according to criteria known to one of skill in the art. Specifically, polypeptides can be fluorescently labeled with compounds such as FITC or rhodamin, essentially as described in Harlow and Lane, Antibodies: A Laboratory Manual, Cold Spring Harbor Laboratory, New York (1988), in particular pages 353-356, or with other fluorescent compounds such as nile red or 2-methoxy-2,4-diphenyl-3(2H)furanone (Daban: Electrophoresis 2001; 22(5): 874-80). Polypeptides can also be labeled with a detectable protein such as GFP (detection based on fluorescence) or the vitamin biotin (detection with streptavidin). Polypeptides can also be radioactively labeled with the isotope S³⁵. Additional methods are widely known in the art.

3. Use of Gene Sequences for Diagnostic Purposes

In certain embodiments, the tissue-specific tumor markers identified herein may be used for the diagnosis of advanced stages of cancer in the given tissue for which the markers are specific. The polynucleotide sequences encoding the tissue specific tumor marker or the polypeptide encoded thereby, where appropriate, may be used in in-situ hybridization or RT-PCR assays of fluids or tissues from biopsies to detect abnormal gene expression. Such methods may be qualitative or quantitative in nature and may include Southern or Northern analysis, dot blot or other membrane-based technologies; PCR technologies; chip based technologies (for nucleic acid detection) and dip stick, pin, ELISA and protein-chip technologies (for the detection of polypeptides). All of these techniques are well known in the art and are the basis of many commercially available diagnostic kits.

In addition, such assays may be useful in evaluating the efficacy of a particular therapeutic treatment regime in animal studies, in clinical trials, or in monitoring the treatment of an individual patient. Such monitoring may generally employ a combination of body fluids or cell extracts taken from normal subjects, either animal or human, under conditions suitable for hybridization or amplification. Standard hybridization may be quantified by comparing the values obtained for normal subjects with a dilution series of a tissue-specific tumor marker gene product run in the same experiment where a known amount of purified gene product is used. Standard values obtained from normal samples may be compared with values obtained from samples from cachectic subjects affected by abnormal gene expression in tumor cells. Deviation between standard and subject values establishes the presence of disease.

Generally, the tissue-specific tumor markers are chosen based on the specificity of their expression in tumors as well as on the high correlation of the reactivity of corresponding antibodies with tumor specimens in ELISA and tissue arrays may be used for development of serological screening procedure. For example, in the context of prostate-specific tumor markers, a large scale analysis of serum and sperm samples obtained from normal donors of different age (before and after 60), patients with different grades and types of prostate carcinoma, androgen dependent and androgen independent, with local, recurrent and metastatic disease, patients with ,tumors of other than prostate origin, as well as patients with noncancerous diseases of prostate may be tested by ELISA on the presence and concentration of the potential candidate polypeptide(s). Then statistical analyses may be performed to evaluate whether the prostate samples express candidate(s) at different levels based on different parameters (histopathological type, Gleason score, tumor size, disease or PSA recurrence).

Once disease is established, a therapeutic agent is administered; and a treatment profile is generated. Such assays may be repeated on a regular basis to evaluate whether the values in the profile progress toward or return to the normal or standard pattern. Successive treatment profiles may be used to show the efficacy of treatment over a period of several days or several-months.

PCR as described in U.S. Pat. Nos. 4,683,195 and 4,965,188 provides additional uses for oligonucleotides specific for the tissue-specific tumor marker genes. Such oligomers are generally chemically synthesized, but they may be generated enzymatically or produced from a recombinant source as described herein above. Oligomers generally comprise two nucleotide sequences, one with sense orientation and one with antisense orientation, employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences. Methods of performing RT-PCR are standard in the art and the method may be carried out using commercially available kits.

Additionally, methods to quantitate the expression of a particular molecule include radiolabeling (Melby et al., J Immunol Methods, 159: 235-244 (1993) or biotinylating (Duplaa et al., Anal Biochem, 229-236 (1993) nucleotides, coamplification of a control nucleic acid, and standard curves onto which the experimental results are interpolated. Quantitation of multiple samples may be speeded up by running the assay in an ELISA-like format where the oligomer of interest is presented in various dilutions and a spectrophotometric or colorimetric response gives rapid quantitation. For example, the presence of abnormal levels of a tissue-specific tumor marker in extracts of biopsied tissues will be indicative of the *onset of a cancer. A definitive diagnosis of this type may allow health professionals to begin aggressive treatment and prevent further worsening of the condition. Similarly, further assays can be used to monitor the progress of a patient during treatment.

4. Hybridization and Detection in Microarrays

Hybridization causes a denatured probe and a denatured complementary target to form a stable nucleic acid duplex through base pairing. Hybridization methods are well known to those skilled in the art (See, e.g., Ausubel, Short Protocols in Molecular Biology, John Wiley & Sons, New York N.Y., units 2.8-2.11, 3.18-3.19 and 4-6-4.9, 1997). Conditions can be selected for hybridization where an exactly complementary target and probes can hybridize, i.e., each base pair must interact with its complementary base pair. Alternatively, conditions can be selected where a target and probes have mismatches but are still able to hybridize. Suitable conditions can be selected, for example, by varying the concentrations of salt in the prehybridization, hybridization and wash solutions, by varying the hybridization and wash temperatures, or by varying the polarity of the prehybridization, hybridization or wash solutions.

Hybridization can be performed at low stringency with buffers, such as 6×SSPE with 0.005% Triton X-100 at 37° C., which permits hybridization between target and probes that contain some mismatches to form target polynucleotide/probe complexes. Subsequent washes are performed at higher stringency with buffers, such as 0.5×SSPE with 0.005% Triton X-100 at 50° C., to retain hybridization of only those target/probe complexes that contain exactly complementary sequences. Alternatively, hybridization can be performed with buffers, such as 5×SSC/0.2% SDS at 60° C. and washes are performed in 2×SSC/0.2% SDS and then in 0.1×SSC. Background signals can be reduced by the use of detergent, such as sodium dodecyl sulfate, Sarcosyl or Triton X-100, or a blocking agent, such as salmon sperm DNA.

After hybridization, the microarray is washed to remove nonhybridized nucleic acids, and complex formation between the hybridizable array elements and the target polynucleotides is detected. Methods for detecting complex formation are well known to those skilled in the art. In a preferred embodiment, the target polynucleotides are labeled with a fluorescent label, and measurement of levels and patterns of fluorescence indicative of complex formation is accomplished by fluorescence microscopy, preferably confocal fluorescence microscopy. An argon ion laser excites the fluorescent label, emissions are directed to a photomultiplier, and the amount of emitted light is detected and quantitated. The detected signal should be proportional to the amount of probe/target polynucleotide complex at each position of the microarray. The fluorescence microscope can be associated with a computer-driven scanner device to generate a quantitative two-dimensional image of hybridization intensity. The scanned image is examined to determine the * abundance/expression level of each hybridized target polynucleotide.

Typically, microarray fluorescence intensities can be normalized to take into account variations in hybridization intensities when more than one microarray is used under similar test conditions. In a preferred embodiment, individual probe/target hybridization intensities are normalized using the intensities derived from internal normalization controls contained on each microarray.

Protein or antibody microarray hybridization is carried out essentially as described in Ekins et al. J Pharm Biomed Anal 1989. 7: 155; Ekins and Chu, Clin Chem 1991. 37: 1955; Ekins and Chu, Trends in Biotechnology, 1999, 17, 217-218; MacBeath and Schreiber, Science 2000; 289(5485): p. 1760-1763.

5. Microarray Expression Profiles

This section describes an expression profile using the polynucleotides of the invention. The expression profile can be used to detect changes in the expression of genes implicated in disease.

The expression profile includes a plurality of detectable complexes. Each complex is formed by hybridization of one or more. polynucleotides of the invention to one or more complementary target polynucleotides. At least one of the polynucleotides of the invention, and preferably a plurality thereof, is hybridized to a complementary target polynucleotide forming at least one, and preferably a plurality, of complexes. A complex is detected by incorporating at least one labeling moiety in the complex as described above. The expression profiles provide “snapshots” that can show unique expression patterns that are characteristic of the presence or absence of a disease or condition.

After performing hybridization experiments and interpreting detected signals from a microarray, particular probes can be identified and selected based on their expression patterns. Such probe sequences can be used to clone a full-length sequence for the gene or to produce a polypeptide.

The composition comprising a plurality of probes can be used as hybridizable elements in a microarray. Such a microarray can be employed in several applications including diagnostics, prognostics and treatment regimens, drug discovery and development, toxicological and carcinogenicity studies, forensics, pharmacogenomics, and the like.

6. Preferred Microarrays of the Invention

The invention provides for microarrays for measuring gene expression characteristic of a cancer of a tissue, comprising at least 4 polypeptide encoding polynucleotides or at least 4 antibodies which bind specifically to the polypeptides encoded by these polynucleotides, as listed in Table 2 and according to the following:

A microarray for measuring gene expression characteristic of breast cancer comprising markers listed in Table 2 sheet 1; A microarray for measuring gene expression characteristic of uterine cancer comprising markers listed in Table 2 sheet 2; A microarray for measuring gene expression characteristic of kidney cancer comprising markers listed in Table 2 sheet 3; A microarray for measuring gene expression characteristic of bladder cancer comprising markers listed in Table 2 sheet 4; A microarray for measuring gene expression characteristic of lung cancer comprising markers listed in Table 2 sheet 5; A microarray for measuring gene expression characteristic of brain cancer comprising markers listed in Table 2 sheet 6; A microarray for measuring gene expression characteristic of colon cancer comprising markers listed in Table 2 sheet 7; A microarray for measuring gene expression characteristic of intestinal cancer comprising markers listed in Table 2 sheet 8; A microarray for measuring gene expression characteristic of stomach cancer comprising markers listed in Table 2, sheet 9; A microarray for measuring gene expression characteristic of liver cancer comprising markers listed in Table 2 sheet 10; A microarray for measuring gene expression characteristic of pancreatic cancer comprising markers listed in Table 2 sheet 11; and A microarray for measuring gene expression characteristic of spleen cancer comprising markers listed in Table 2 sheet 12.

B. Immunodiagnosis and Polypeptide Detection

In certain embodiments, antibodies may be used in characterizing the tissue-specific tumor marker content of healthy and diseased tissues, through techniques such as ELISAs, immunohistochemical detection and Western blotting.

This may provide a screen for the presence or absence of malignancy or as a predictor of future cancer. Once the tissue-specific tumor marker is identified, one of skill in the art may produce antibodies against that marker using techniques well known to those of skill in the art

The use of such antibodies in an ELISA assay is contemplated. For example,: such antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the biological sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for the tumor marker that differs from the first antibody. Appropriate conditions preferably include diluting the sample with diluents such as BSA, bovine gamma globulin (BGG) and phosphate buffered saline (PBS)/Tween. These added agents also tend to assist in the reduction of nonspecific background. The layered antisera is then allowed to incubate for from about 2 to about 4 hr, at temperatures preferably on the order of about 25° C. to about 27° C. Following incubation, the antisera-contacted surface is washed so as to remove non-immunocomplexed material. A preferred washing procedure includes washing with a solution such as PBS/Tween, or borate buffer.

For convenient detection purposes, the second antibody may preferably have an associated enzyme that will generate a color development upon incubating with an appropriate chromogenic substrate. Thus, for example, one will desire to contact: and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and hydrogen peroxide, in the case of peroxidase as the enzyme- label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

Immunoblotting and immunohistochemical techniques using antibodies directed against the tumor markers also are contemplated by the invention. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

Flow cytometry methods also may be used in conjunction with the invention. Methods of performing flow cytometry are discussed in Zhang et al., J Immunology, 157:3980-3987 (1996) and Pepper et al., Leuk. Res., 22(5):439-444 (1998). Generally, the cells, preferably blood cells, are permeabilized to allow the antibody to enter and exit the cell. If the gene in question encodes a cell surface protein, the step of permeabilization is not needed. After permeabilization, the cells are incubated with an antibody. In preferred embodiments, the antibody is a monoclonal antibody. It is more preferred that the monoclonal antibody be labeled with a fluorescent marker. If the antibody is not labeled with a fluorescent marker, a second antibody that is immunoreactive with the first antibody and contains a fluorescent marker. After sufficient washing to ensure that excess or non-bound antibodies are removed, the cells are ready for flow cytometry. If the marker is an enzyme, the reaction monitoring its specific enzymatic activity either in situ or in body fluids may be performed.

Determining the level of a polypeptide in a sample for the purposes of diagnosis may also be carried out in the form of enzymatic activity testing, when the polypeptide being examined offers such an option.

In addition, whole body image analysis following injection of labeled antibodies against cell surface marker proteins is a diagnostic possibility, as described above; the detected concentrations of such antibodies are indicative of the sites of tumor/ metastases growth as well as their number and the tumor size.

C. Carcinogenicity Testing

The tissue specific tumor marker genes identified using the methods of the invention can form the basis of a carcinogenicity test. Test agents are evaluated to see if their effects on human cells mimic the effects of loss of the tumor suppressor. Thus the agents are in essence being evaluated for the ability to induce a tumor suppressor mutation, or a mutation in another gene which is in the same regulatory pathway, or a non-genetic effect which mimics tumor suppressor loss. Test agents which are found to have at least some of the same constellation of effects as tumor suppressor loss on the regulation of the genes identified herein to be tumor suppressor-regulated, are identified as potential carcinogens. Any single gene identified can be used, as can at least 2, 5, 10, 15, 20, 25, 30, 35, 40, 45, 50, 75, 100, 125, or 150 or more genes identified herein.

The invention also contemplates the use of the tissue-specific tumor markers identified herein in the screening of compounds for activity in either stimulating tumor suppressor activity, overcoming the lack of a tumor suppressor, or blocking the effect of a mutant tumor suppressor molecule. It is contemplated that any agent which decreases the expression of a tissue specific tumor marker that was up-regulated upon tumor suppressor inactivation may serve as an anti-tumor agent. Screening assays for such agents are well known to those of skill in the art. U.S. Pat. No. 6,262,242 is incorporated herein by reference as providing a general teaching of such screening assays and others relating to the diagnostic and therapeutic uses of tumor related genes.

II. Therapeutic Methods of Using Identified Markers

The genes identified by the invention herein as down-regulated by the loss of a tumor suppressor may prove effective against a given cancer when delivered therapeutically to the cancer cells. Antisense constructs of the genes identified herein as up-regulated as a result of loss of tumor suppressor can be delivered therapeutically to cancer cells. Other therapeutic possibilities include siRNA or small molecules or antibodies inhibiting the target protein function and/or expression. The goal of such therapy is to retard the growth rate of the cancer cells. Expression of the sense molecules and their translation products or expression of the antisense mRNA molecules has the effect of inhibiting the growth rate of cancer cells or inducing apoptosis. Sense nucleic acid molecules are preferably delivered in constructs wherein a promoter is operatively linked to the coding sequence at the 5′-end and initiates transcription of the coding sequence. Anti-sense constructs contain a promoter operatively linked to the coding sequence at the 3′-end such that upon initiation of transcription at the promoter an RNA molecule is transcribed which is the complementary strand from the native MRNA molecule of the gene.

Delivery of nucleic acid molecules can be accomplished by many means known in the art. Gene delivery vehicles are available for delivery of polynucleotides to cells, tissue, or to a mammal for expression. For example, a polynucleotide sequence of the invention can be administered either locally or systemically in an expression construct or vector. There are a number of ways in which expression vectors may be introduced into cells. In certain embodiments of the invention, the expression construct comprises a virus or engineered construct derived from a viral genome. In other embodiments, non-viral delivery is contemplated. The ability of certain viruses to enter cells via receptor-mediated endocytosis, to integrate into host cell genomes and express viral genes stably and efficiently have made them attractive candidates for the transfer of foreign genes into mammalian cells (Ridgeway, In: Vectors: A survey of molecular cloning vectors and their uses Rodriguez R L, Denhardt D T, eds. Stoneham: Butterworth, pp. 467-492,. 1988; Nicolas et al., In: Vectors: A survey of molecular cloning vectors and their uses, Rodriguez & Denhardt (eds.), Stoneham: Butterworth, pp. 493-513, 1988; Baichwal et al., In: Gene Transfer, Kucherlapati ed., New York, Plenum Press, pp. 117-148, 1986; Temin, In: gene Transfer, Kucherlapati (ed.), New York: Plenum Press, pp. 149-188, 1986). The viral vector can also be an astrovirus, coronavirus, orthomyxovirus, parvovavirus, paramyxovirus, parvovirus, picomavirus, poxvirus, togavirus viral vector. See generally, Jolly, Cancer Gene Therapy 1:51-64 (1994); Kimura, Human Gene Therapy 5:845-852 (1994), Connelly, Human Gene Therapy 6:185-193 (1995), and Kaplitt, Nature Genetics 6:148-153 (1994).

Several non-viral methods for the transfer of expression constructs into cultured bacterial cells are contemplated by the invention. This section provides a discussion of methods and compositions of non-viral gene transfer. DNA constructs of the invention are generally delivered to a cell and, in certain situations, the nucleic acid or the protein to be transferred may be transferred using non-viral methods. The non-viral methods include calcium phosphate precipitation (Graham et al., Virology, 52:456-467, 1973; Chen et al., Mol. Cell. Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA, 81:7161-7165, 1984), direct microinjection (Harland and Weintraub, J. Cell Biol., 101:1094-1099, 1985.), DNA-loaded liposomes (Nicolau and Sene, Biochim. Biophys. Acta, 721:185-190, 1982; Fraley et al., Proc. Natl. Acad. Sci. (USA), 76:3348-3352, 1979; Felgner, Sci Am. 276(6):102-6, 1997; Felgner, Hum Gene Ther. 7(15):1791-3, 1996), cell sonication (Fechheimer et al., Proc. Natl. Acad. Sci. (USA), 84:8463-8467, 1987), gene bombardment using high velocity microprojectiles (Yang et al., Proc. Natl. Acad. Sci USA, 87:9568-9572, 1990), conjugation (Gavigan et al. In: Mycobacteria Protocols, Tanya Parish and Neil G. Stoker (eds). pp. 119-128 1998. Humana Press, Twtowa, N.J.) and receptor-mediated transfection (Wu et al., J. Biol. Chem., 262:4429-4432, 1987; Wu et al., Biochemistry, 27:887-892, 1988; Wu et al., Adv. Drug Delivery Rev., 12:159-167, 1993).

The expression construct also may be entrapped in a liposome. Liposomes that can act as gene delivery vehicles are described in U.S. Pat. No. 5,422,120, PCT Patent Publication Nos. WO 95/13796, WO 94/23697, and WO 91/144445, and EP No. 524,968. The addition of DNA to cationic liposomes causes a topological transition from liposomes to optically birefringent liquid-crystalline condensed globules (Radler et al., Science, 275(5301):810-4, 1997). These DNA-lipid complexes are potential non-viral vehicles for use in gene delivery.

Also contemplated in the invention are various commercial approaches involving “lipofection” technology. In certain embodiments of the invention, the liposome may be complexed with a hemagglutinating virus (HVJ). This has been shown to facilitate fusion with the cell membrane and to promote cell entry of liposome-encapsulated DNA (Kaneda et al., Science, 243:375-378, 1989). In other embodiments, the liposome may be complexed or employed in conjunction with nuclear nonhistone chromosomal proteins (HMG-1) (Kato et al., J. Biol. Chem., 266:3361-3364, 1991).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu et al., 1987, supra) and transferrin (Wagner et al., Proc. Natl. Acad Sci. USA, 87(9):3410-3414, 1990). Recently, a synthetic neoglycoprotein, which recognizes the same receptor as ASOR, has been used as a gene delivery vehicle (Ferkol et al., FASEB J., 7:1081-1091, 1993; Perales et al., Proc. Natl. Acad. Sci. USA, 91:4086-4090, 1994) and epidermal growth factor (EGF) has also been used to deliver genes to squamous carcinoma cells (Myers, EPO 0273085).

Another embodiment of the invention for transferring a naked DNA expression construct into cells may involve particle bombardment. This method depends on the ability to accelerate DNA coated microprojectiles to a high velocity, allowing them to pierce cell membranes and enter cells without killing them (Klein et al., Nature, 327:70-73, 1987). Exemplary naked DNA introduction methods are described in PCT Patent Publication No. WO 90/11092 and U.S. Pat. No. 5,580,859. Several devices for accelerating small particles have been developed. One such device relies on a high-voltage discharge to generate an electrical current, which in turn provides the motive force (Yang et al. Proc. Natl. Acad. Sci. USA, 87:9568-9572, 1990). The microprojectiles used to date have consisted of biologically inert substances such as tungsten or gold beads.

### EXAMPLE 1

Validation of the Methods of the Invention in LNCAP Cells

The present Example demonstrates the methods of identifying tissue-specific tumor markers that are negatively regulated by a tumor suppressor. In the present Example, it was demonstrated for the first time that the expression of PSA is negatively regulated by p53.

Prostate cancer, the most frequently diagnosed malignancy in men in western countries (Cancer, 71(Suppl.): 880-886, 1993), is often characterized by elevated prostate-specific antigen (PSA) secretion that is broadly used as a blood-borne diagnostic marker of the disease. PSA is synthesized exclusively in prostate epithelia by normal, hyperplastic and malignant cells, and its levels are seen to rise several-fold above background in the blood as a result of benign prostatic hyperplasia. The levels of PSA in the serum of individuals at end-stage metastatic prostate carcinoma may be more than a hundred times higher than normal levels of the marker (Kim and Logothetis, Urol. Clin. North Am., 26:281-290 1999; Abate-Shen and Shen, Genes Dev., 14:2410-2434, 2000). Expression of the PSA gene was demonstrated to be directly regulated by binding of androgen receptor (AR) (Young et al., Cancer Res., 51:3748-3752 1991; Montgomery et al., Prostate, 21:63-73, 1992; Trapman and Cleutjens, Semin. Cancer Biol., 8:29-36 1997) to three androgen responsive elements (AREs) identified within the 5.8 kb PSA promoter (Schuur et al., Urology, 162:2040-2045 1996; Cleutjens et al., Mol. Endocrinol., 11:148-161, 1997; Zhang et al., Biochem. Biophys. Res. Comm., 231:784-788, 1997; Zhang et al., Nucleic Acids Res., 25:3143-3150, 1997). However, detailed analyses of PSA promoter activity in androgen-dependent and androgen-independent prostate carcinoma cell lines indicated that the control of transcription of the PSA gene is not limited to androgen regulation (Yeung et al., J. Biol. Chem., 275:40846-40855 2000).

The present Example provides evidence showing the involvement of p53 tumor suppressor in regulation of PSA promoter. Micro-array hybridization and analyses were performed using LNCaP cells. This cell line originally isolated from lymph node metastases of prostate adenocarcinoma, retains wild type p53, androgen dependence and expression of a variety of prostate-specific markers, all known as properties of a relatively early stage of prostate cancer progression. Inactivation of p53 function by a dominant negative mutant in these cells imitates an important step in tumor progression and allows analysis of the genetic basis for altered tumor cell phenotype associated with p53 suppression.

A variant of LNCaP cells with inactivated p53, LN-56 (Rokhlin et al., Oncogene, 19:1959-1968, 2000), was generated by transduction of retroviral construct expressing the potent dominant negative p53 mutant, GSE56 (Ossovskaya et al., Proc. Natl. Acad. Sci. USA, 93:10309-10314, 1996). GSE56-mediated inactivation of p53 resulted in resistance to apoptosis and increased tumorigenicity of LN-56 cells (Rokhlin et al., Oncogene, 19:1959-1968, 2000), suggesting that p53 is at least partially functional in LNCaP cells. Moreover, both steady-state and inducible expression level of p53-responsive gene p21/waf1 were reduced in LN-56 cells.

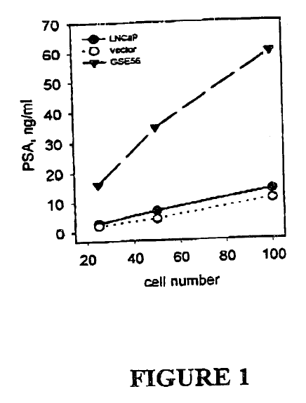

PSA was among the genes that showed the most pronounced differential expression in LNCaP versus LN-56 cells. PSA was expressed four times higher in LN-56 than in LNCaP cells. To determine whether differences in mRNA expression correlated with PSA protein expression, the amount of secreted PSA in the medium from LNCaP and LN-56 cells was determined. This revealed that the latter cells produced 6-8 times more PSA as compared to LNCaP (FIG. 1). These observations suggested that expression of PSA gene is likely to be under the negative regulatory control of p53 and that elevated expression of PSA in advanced prostate cancer may be indicative for p53 suppression.

To determine whether p53 directly affects transcription from the PSA promoter, a CAT assay was performed in the LNCaP cells transfected with the reporter constructs containing the CAT gene under the control of the proximal PSA promoter (nucleotides −407 to +11) linked to the PSA enhancer element (nucleotides −5322 to −3740). This construct was previously shown to imitate the endogenous PSA gene regulation (Zhang et al., Biochem. Biophys.. Res. Comm., 231:784-788, 1997; Zhang et al., Nucleic Acids Res., 25:3143-3150, 1997). Since PSA transcription is also known to be androgen dependent, for these studies, LNCaP cells that retain androgen dependence were used. To increase the wild type p53 activity, different amounts of wild type p53 expression plasmid were cotransfected with the PSA-reporter vector. To inhibit endogenous p53 function, cotransfection with the GSE56-expressing plasmid was employed (Ossovskaya et al., Proc. Natl. Acad. Sci. USA, 93:10309-10314, 1996). Another reporter construct containing the CAT reporter gene under the control of p53-responsive promoter carrying the p53-binding site from p21/Waf1 gene was used to monitor the p53 activity in transfected cells.

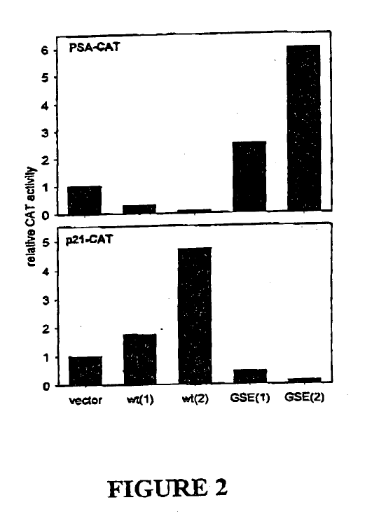

Introduction of different amounts of wild type p53-expressing plasmid into LNCaP cells resulted in dose-dependent changes of CAT activity driven from both p21- and PSA-derived promoter elements, though in opposite directions: while the p21 promoter construct was activated, expression of the PSA reporter was suppressed by p53. When, GSE56 was co-transfected with either of the reporter constructs, an inverted picture was observed (FIG. 2).

Sequence analysis does not reveal any canonical p53 binding sites within or in the vicinity of the PSA promoter region and in the first intron of the PSA gene. It is noteworthy that most of the known p53-repressed genes also do not possess such sites in their promoter regions and do not necessarily require p53.

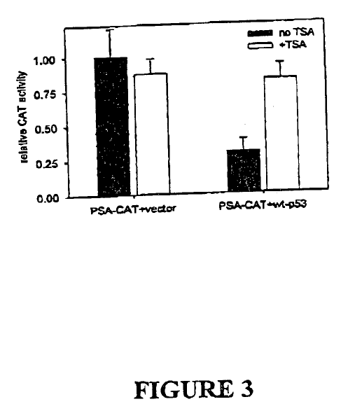

Since it has previously been shown that negative regulation of transcription by p53 may involve p53-mediated recruitment of histone deacetylases (HDAC) (Murphy et al., Genes Dev., i3:2490-2501 1999), the inventors set out to determine whether this would be also true for the PSA promoter. Following co-transfection of PSA-CAT reporter and wild type p53-expressing plasmids, the LNCaP cells were treated with the HDAC inhibitor trichostatin A (TSA) for 24 h and the lysates of transfected cells were tested for CAT activity. These experiments demonstrated that TSA completely abrogated the p53-mediated repression of PSA promoter driven transcriptional activity. At the same time, TSA had no effect on p53-mediated transactivation as determined in a similar experiment employing the p21 promoter-driven CAT reporter construct (FIG. 3). Thus, the PSA gene can be added to a growing list of genes that are negatively regulated by p53 through HDAC-mediated transcriptional repression.

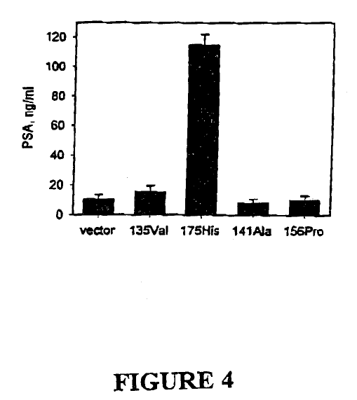

Use of a potent dominant negative p53 inhibitor, GSE56, allowed the determination of the p53 dependence of PSA expression. However, this mutant form does not naturally occur in human tumors. In order to more adequately imitate events naturally occurring in the course of tumor progression, the effect of four tumor-derived p53 mutants (135Val, 141Ala, 156Pro and 175His), two of which are frequent types of p53 mutants in prostate cancer (141Ala and 175His), on expression of PSA was determined. LNCaP cells were transduced with retroviruses expressing the above p53 mutant variants and the level of PSA was measured in the medium conditioned by each type of the transduced cell populations. In parallel, the potential suppressive effect of the introduced p53 mutants on the activity of endogenous p53 in LNCaP cells was estimated by monitoring the p53-dependent p21 induction in response to doxorubicin treatment (Rokhlin et al., Oncogene, 19:1959-1968, 2000). As seen in FIG. 4, only one of the tested mutants, 175His, displayed a strong dominant negative activity against the wild type p53 reflected by the lack of p21 induction by DNA damage. Val135 mutant showed marginal p53 suppression, while the two remaining mutants did not interfere with p53-mediated p21 induction at all. Remarkably, this pattern of anti-p53 activity was exactly mirrored in the pattern of PSA expression: compared to control, 175His expressing cells produced 9-11 times more PSA, whereas in 135Val cells its level was slightly increased and 141Ala, 156Pro.

The list of genes whose expression was changed following wild type p53 suppression in LNCaP cells is attached as Table 1.

In conclusion, this Example demonstrate that the transcription of PSA gene in the prostate carcinoma cell line, LNCaP, is under strict negative control of p53 and its expression can be greatly activated by suppression of wild type p53 activity. Since LNCaP is considered most adequate and conventional among available in vivo models of hormone-dependent prostate cancer, these results likely reflect regulation of PSA in naturally occurring tumors. Thus, one of the most useful diagnostic tumor markers is, in fact, a tissue specific indicator of p53 inactivation in prostate cells. Being dependent on p53 inactivation, elevated production of PSA may therefore be indicative for the ongoing selection of p53-deficient cell variants with the broken control of apoptosis, angiogenesis, and genomic stability, all normally regulated by wild type p53. In fact, the loss of functional p53 by LNCaP cells is accompanied not only by elevated PSA secretion but also by acquisition of high tumorigenicity and resistance to TNF (Rokhlin et al., Oncogene, 19:1959-1968, 2000).

For further detail concerning the above Example, see the inventors' publication: Gurova et al: Expression of prostate specific antigen (PSA) is negatively regulated by p53. Oncogene 2002, 21: 153-157.

### EXAMPLE 2

Validation of the Methods of the Invention in Sets of p53-/- and p53 Wild-Type Tissues and Identification of New Cancer Markers

The most desirable characteristics of an ideal tumor marker involve tissue/organ specificity of expression and association with definite type of tumor and/or stage of tumor progression. Alternatively, tumor markers may be ubiquitously highly expressed in numerous tumors displaying low expression or lack of expression in normal tissues. Prospective markers can be oncogenes themselves, and thus be directly involved in malignant transformation (i.e., BCR-ABL in Ph′-positive CML and ALL) (Daley et al., Science Feb. 16, 1990; 247(4944):824-30.) On the other hand, the marker genes may be not the active players in carcinogenesis, their overexpression being a consequence of transformation-associated changes in gene regulation. Genes from the first group may be targets for functional inhibition via direct targeting by drugs, whereas the genes(proteins) from the second group, if localized to the plasma membrane, may be used for targeting of tumor cells via specific antibodies-mediated strategies. Changes in the expression of these genes may also be used as a readout for the establishment of bioassay for the purpose of screening for anti-cancer drugs, e.g. targeted at reactivation of normal tumor suppressor gene function. Marker proteins from both groups may serve also as early diagnostic or progressive tumor markers if found in body fluids (i.e., like PSA in the cancer of prostate). Alternatively they may serve as differential diagnosis markers during morphological examination of tumor samples or tissue biopsies.

The invention provides a systematic approach for the search of cancer marker genes. This approach is based on the idea that many such genes may be transcriptionally activated in tissues following the loss of the most common tumor suppressor genes like e.g., p53, PTEN, RB, and p16/p19 and that this regulation will be conserved in normal and tumor cells from the same origin. Technically, the gene discovery may be performed by comparison of gene expression profiles in the fitted tissue pairs derived from normal and genetically modified mice, like i.e. p53-/- mice, p16/p19-/- mice, tissues with targeted expression of SV40 large T antigen that simultaneously inactivates both RB and p53 function (TRAMP mice, expressing LT-Ag in prostate). There are some literature indications, as well as examples that support the feasibility of such an approach. For example, it was demonstrated wild-type p53 can suppress the expression of two neoangiogenesis and progression-related genes known to be highly expressed in tumors, COX2 (Subbaramaiah et al., J Biol Chem Apr. 16, 1999; 274(16):10911-5) and VEGF (Zhang et al., Cancer Res Jul. 1, 2000; 60(13):3655-61.) Both genes are currently regarded as targets for anti-cancer therapeutics. However, the connection of COX2 and VEGF to p53 was found long after they were first discovered and their function and tumor association were well established.

In the present studies presented in Example I above, the inventors created an isogenic pair of LNCaP prostate tumor cell lines differing in their p53 status and applied cDNA microarray analysis to look for differentially expressed genes. It was discovered that the baseline expression of several known tumor markers is significantly elevated in LNCaP cells that lack functional p53 protein compared to the same cells that express wt p53. These genes include e.g., COX2, tumor-specific heparin-binding growth factor midkine (possesses angiogenic and anti-apoptotic properties) (Ikematsu et al., Br J Cancer September 2000 83(6):701-6), tumor tissue associated hyaluronan receptor CD44 (Sneath et al., Mol Pathol. August 1998; 51(4):191-200) and PSA (prostate specific antigen).

COX2 inhibitors are currently in clinical trials against prostate cancer. Midkine was immunohistochemically shown to be expressed specimens 86.3% of prostate cancer specimen examined, with metastatic lesions generally showing higher expression than the corresponding primaries; normal prostate tissues were negative or showed only weak staining. Midkine was also detected in 12 of 15 latent cancers (80%) and in 12 of 16 cases of PIN (75%) (Konishi et al., Oncology October 1999; 57(3):253-7). PSA is the major currently used prostate cancer diagnostic marker. In Example 1 it is shown that its promoter is directly suppressed by wt p53, thus PSA up-regulation in prostate cancer may be indicative for the wt p53 loss. The list of genes which expression was changed following wt p53 suppression in LNCaP cells is attached in the accompanying Excel file (Table 1). The inventors concluded that general proof of concept is achieved and embarked upon a large-scale experiment involving microarray-based comparison of gene-expression profiles in the tissue pairs derived from normal and p53-/- mice.

Poly A RNA was extracted from spleen, pancreas, liver, stomach, intestine, colon, lung, brain, bladder, kidney, placenta/uterus and mammary glands of normal and p53-deficient mice and used for fluorescently-labeled probes for microarray hybridizations. All tissue-specific probes were labeled with Cy5 fluorescent marker, while the common control probe (an equal proportion mixture of all the RNAs) was labeled with Cy3. The common control probe was used in order to assess also the tissue-specificty of gene expression. All probes were hybridized to MouseGEM (Incyte). Upon quality control and pair-wise balancing of Cy5 and Cy3 signals, the differential (against common control) gene expression levels were normalized between p53-/- tissues and their corresponding normal counterparts. As a result the inventors obtained a table of genes containing their differential expression levels in p53-/- tissues compared to the corresponding normal tissues; the genes were sorted according to their expression levels in one particular tissue from maximally up-regulated genes to maximally down-regulated ones (Table 2). Genes showing absolute differential expression levels less than 1.9 were excluded from these tables. Thus, these tables contain the lists of genes with maximal differential tissue-specific expression in p53-deficient mice. It must be noted, that the majority of identified genes has changed their expression in a tissue-specific manner, though some of them like, e.g., choline kinase (known to be up-regulated and activated in numerous cancer types) was up-regulated in p53-/- pancreas, stomach, intestine, lung, bladder, uterus, and mammary gland. Another interesting observation is that there was almost no overlap between the list of genes that were up- or down-regulated in different p53-/- tissues.

Out of the approximately 10,000 genes printed on the microarray, approximately 445 genes that were found to be up-regulated in p53-/- tissues were studied in further detail, as they had the largest potential of serving as tumor markers (drug targets and diagnostic markers). These genes appear in the Excel file, Table 3. This table combines the p53-dependent differential expression data with the tissue specificity of gene expression data. The actual differential expression of genes in regard to common control is also presented. As discussed above, the markers are based on genes that are: either

1. up-regulated in a certain p53-/- tissue and are normally expressed predominantly in this tissue; or

2. normally expressed at low levels in one or several tissues but are up-regulated in one or numerous p53-/- tissues.

As evident from Table 3, genes belonging to both groups were identified. For example, Mest-linked imprinted transcript, anonymous brain protein, -and potassium voltage-gated channel (subfamily Q,. member2) are specifically expressed in brain and are up-regulated in p53-/- brain compared to the normal one. Another example: expression of liver-specific fatty acid transporter, betaine-homocystein methyltransferase and of several unknown genes (ESTs) is significantly increased in p53-/- hepatic tissue. On the other hand, genes such as choline kinase that is usually expressed at low levels is significantly enhanced in numerous p53-/- tissues (see above). A similar behavior is also observed i.e. for EGF (enhanced in p53-/- bladder and mammary gland); carbonic anhydrase 6 (enhanced in p53-/- bladder); zinc finger protein 101 (enhanced in p53-/- liver). Numerous unknown genes (ESTs) also fall in this the most promising category.

The approximately 445 genes identified as up-regulated in p53-/- mice were further prioritized for the purpose of serving as diagnostic markers; the highly preferred diagnostic markers are presented in Table 5 (general cancer markers) and Table 6 (tissue-specific cancer markers). Thus, of the approximately 10,000 genes printed on the array, the inventors were able to select through the methods of the invention a total of 338 genes ideally suited for several diagnostic and prognostic uses in various cancers, as described herein.

Table 4 sheet 1 provides a list of 32 genes/ polypeptides identified according to the methods of the present example as disclosed herein that are known in the art to be markers for certain cancers, thus validating the effectiveness of the methods of the invention. This table also includes the PubMed indexing numbers of publications that disclose the connection of these genes/ polypeptides to cancer.

In summary, the inventors provide a list of genes characterized in regard to tissue-specificity of their normal expression and induction/reduction in various p53-deficient tissues. A similar expression pattern should be preserved in tumor cells originating from the same tissue. Thus, the identified genes may serve as tumor markers.

### EXAMPLE 3

Validation Data for Additional Tumor Suppressor Genes

The methods of the invention, as validated in example 2, are not limited only to the use of the tumor suppressor p53, as any other tumor suppressor gene with confirmed involvement in a specific type of cancer may be involved in negative regulation of tissue specific genes by direct (i.e., transcription factors) or indirect (i.e., signaling pathway members) pathways.

The inventors therefore proceeded to test these methods on p53 knockout mice, TRAMP mice and PTEN hemisigous mice (the complete knockout is non-viable). TRAMP transgenic mice express large T-antigen of SV40 under the control of prostate-specific probasin promoter (Jackson labs), and have both tumor suppressor genes p53 and Rb inactivated. PTEN hemisigous mice have only one allele of the tumor suppressor gene PTEN. The experiments were carried out on prostate cells. For each hybridization, RNA was isolated from prostates of 6-8 males of different age in dependence of genotype prior to appearance of initial signs of hyperplaysia of prostate according to published data (p53KO, TRAMP and corresponding control C57BL6 mice - 9-10 weeks old, PTEN and corresponding control FVB mice—6 weeks old). Total RNA was isolated from each prostate separately from 6-8 animals of each genotype. In total two probes for each genotype were prepared and hybridized with a set of three mouse Affymetrix arrays which cover the majority of known mouse transcripts. Genes with reproducible 2 fold overexpression in tumor suppressor gene deficient prostates as compared with wild type organs (confirmed specific hybridization) in both two repetitive hybridizations were picked for identification of human homologs.

Remarkably, among 161 genes picked for further analysis, more than 10 were found to be either known or candidate cancer markers although their p53 or Rb dependence had not been previously determined (see Table 4 sheet 2). A significant proportion of other genes that came out of these experiments are known as genes with melanoma or glioma-specific expression that is consistent with frequent acquisition of traces of neuroendocrinal differentiation by prostate cancer cells. Additional genes/ polypeptides previously linked to cancer and identified according to the method described in this Example include: KIAA430, limkainb1 (NP_{—}596912), that associates with the LIM-kinase 2, which may be critical for metastasis (PMID: 11208874); Glutamate-cystein ligase (modifier subunit), the rate-limiting enzyme in glutathion synthesis, that is overexpressed in numerous tumor types (PMFD: 11774239, 11753966); PCNA (proliferating cells nuclear antigen), an auxiliary protein of DNA polymerase delta that is involved in the control of eukaryotic DNA replication, and overexpressed in numerous cancer types (e.g., PMID: 12145573, 12046056, PMID: 11750711, 11606074); Mcmd5, a DNA replication licensing factor under transcriptional control of E2F (PMID: 10327050), (abolishment of Rb function by TRAMP), which is a known marker for cancer (PMID: 2122098, 11839717, 10551502, 9843993); Transducin-like enhancer protein 2 (TLE2), a Nuclear effector molecule and neural/neuroectodermal associated gene overexpressed in synovial, sarcoma (PMID: 12414507); Inhibitor of DNA binding 1 (ID1), a negative regulator of helix-loop-helix. DNA binding proteins with the following functions: required to maintain the timing of neuronal differentiation in the embryo and invasiveness of the vasculature (hence, neurogenesis and vasculogenesis) (PMID: 10537105), inhibits transcription of trombospondin-1, thus promoting angiogenesis (PMID: 12498716), helps to, keep neuroblastoma cells in an undifferentiated state (PMID: 11756408), directly inhibits expression of p16 via repression of Ets and E-protein-mediated transactivation (PMID: 11427735), trichostatin A treatment of ovarian cancer cells causes decrease of Rb phosphorylation and reduction of ID1 expression (thus the observed expression pattern in TRAMP mice is concomitant with Rb inactivation by T-Ag) (PMID: 12479699). This gene has several known associations to cancer: associated with grade and invasiveness of endometrial carcinoma (PMID: 11275368), upregulated in early melanomas (if not, p16 is mutated) (PMID: 11507043), expressed in astrocytes and endothelial cells within astrocytomas positively correlating with stage and grade (PMID: 12007145).

These preliminary results clearly support the main concept of the instant invention, and demonstrate that cancer markers can be frequently found among the genes that are normally under the negative control of tumor suppressors.

Table 4 sheet 2 provides a list of 12 genes/ polypeptides identified according to the methods of: the present Example and of Example 2 as disclosed herein that are known in the art to be markers for certain cancers, thus validating the effectiveness of the methods of the invention. This table also includes the PubMed indexing numbers of publications that disclose the connection of these genes to cancer.

While the invention has been described in terms of specific embodiments, it is understood that variations and modifications will occur to those skilled in the art. Accordingly, only those limitations appearing in the appended claims should be placed upon the invention. The entire disclosure of all publications and patents cited herein are hereby incorporated by reference.

TABLE 1

| RowType | GeneDescription | GeneID | LN56_DIFF | |
|---|---|---|---|---|
| gene | Human_clone_23586_mRNA_sequence_{Incyte_PD:530629} | 530629 | 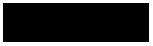 | |
| gene | ESTs,_Moderately_similar_to_!!!!_ALU_SUBFAMILY_J_WARNING_ENTRY_!!!!_[H. sapiens]_{Incyte_PD:855928} | 855928 | 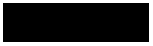 | |
| gene | Fc_fragment_of_IgG,_low_affinity_IIIa,_receptor_for_(CD16)_{Incyte_PD:1560730} | 1560730 | 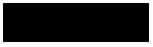 | |
| gene | prostaglandin- endoperoxid_synthase_2_(prostaglandin_G/H_synthase_and_cyclooxygenase)_Inc yte_PD:3139163} | 3139163 | 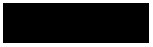 | COX2 |
| gene | Human_GABA-A_receptor_pi_subunit_mRNA,_complete_cds_{Incyte_PD:1824443} | 1824443 | 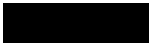 | glutamate receptor (see glioma's paper and my text about prostate) |
| gene | kallikrein_3,_(prostate_specific_antigen)_{Incyte_PD:1655492} | 1655492 | 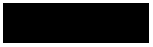 | PSA |
| gene | regulator_of_G-protein_signalling_2,_24kD_{Incyte_PD:1218114} | 1218114 | 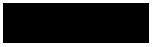 | |
| gene | Homo_sapiens_mRNA_for_KIAA0540_protein,_partial_cds_{Incyte_PD:2966l26} | 2966126 | 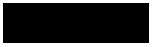 | novel, sequence is not complete, contains Glutamine amidotransferases class-II signature and Beach domain (PFAM-did not find functional description) |
| gene | diaphorase_(NADH/NADPH)_(cytochrome_b-5_reductase)_{Incyte_PD:2637222} | 2637222 | 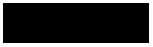 | |
| gene | Human_tumor_necrosis_factor-inducible_(TSG- 6)_mRNA_fragment,_adhesion_receptor_CD44_putative_CDS_{Incyte_PD:3142364} | 3142364 | 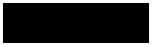 | hyaluronan receptor |
| gene | ESTs,_Moderately_similar_to_!!!!_ALU_SUBFAMILY_SC_WARNING_ENTRY_!!!!_[H.sapiens]_{Incyte_PD:2458029} | 2458029 | 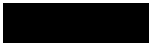 | |
| gene | ESTs_{Incyte_PD:1305355} | 1305355 | 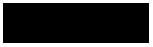 | |
| gene | ESTs_{Incyte_PD:5740665} | 2740665 | 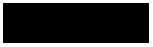 | |
| gene | butyrylcholinesterase_{Incyte_PD:1599272} | 1599272 | 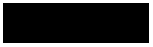 | |
| gene | ESTs,_Weakly_similar_to_X- linked_retinopathy_protein_H.sapiens]_{Incyte_PD:2311432} | 2311432 | 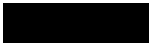 | |
| gene | ESTs_{Incyte_PD:2423162} | 2423162 | 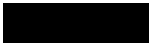 | |
| gene | ESTs_{Incyte_PD:1965673} | 1965673 | 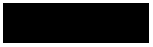 | |
| gene | ESTs_{Incyte_PD:3519565} | 3519565 | 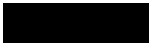 | |
| gene | ESTs_{Incyte_PD:1537925} | 1537925 | 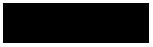 | |
| gene | batericidal/permeability-increasing_protein_{Incyte_PD:406016} | 406016 | 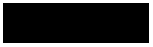 | |
| gene | oligophrenin_1_{Incyte_PD:42165201 | 4216520 | 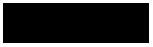 | |
| gene | ESTs_{Incyte_PD:944140} | 944140 | 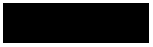 | |
| gene | KIAA0025_gene_product_{Incyte_PD:2054420} | 2054420 | 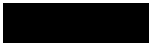 | |
| gene | ESTs,_Moderately_similar_to_!!!!_ALU_SUBFAMILY_SP_WARNING_ENTRY_!!!!_[H.sapiens]_{Incyte_PD:1574536} | 1574536 | 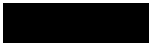 | |
| gene | ESTs_{Incyte_PD:9669481 | 966948 | 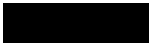 | |
| gene | ESTs,_Weakly_similar_to_(defline_not_available_4589652)_(H.sapiens]_{Incyte_PD: 3749894} | 3749894 | 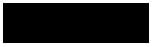 | |
| gene | ESTs_{Incyte_PD:2045819} | 2045819 | 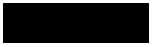 | |
| gene | ESTs_{Incyte_PD:979945} | 979945 | 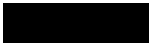 | |
| gene | ESTs_{Incyte_PD:2149058} | 2149058 | 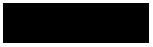 | |
| gene | prostaglandin_E_receptor_4_(subtype_EP4)_{Incyte_PD:1631793} | 1631793 | 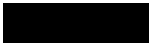 | |
| gene | ESTs,_Moderately_similar_to_!!!!_ALU_SUBFAMILY_SX_WARNING_ENTRY_!!!!_[H.sapiens]_{Incyte_PD:2447969} | 2447969 | 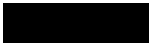 | |
| gene | ESTs_{Incyte_PD:1397926} | 1397926 | 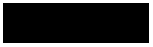 | |
| gene | frizzled_(Drosophila)_homolog_5_{Incyte_PD:3129290} | 3129290 | 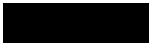 | |
| gene | ATPase,_Na+/K+_transporting,_alpha_2_(+)_polypeptide_{Incyte_PD:1622542} | 1622542 | 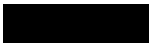 | |
| gene | Human_desmocollin-2_mRNA,_3′_UTR_{Incyte_PD:1403294} | 1403294 | 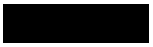 | |
| gene | N-acetylgalactosaminidase,_alpha-_{Incyte_PD:1664863} | 1664863 | 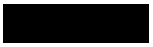 | |
| gene | protease_inhibitor_12_(neuroserpin)_{Incyte_PD:2716511} | 2716511 | 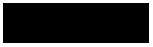 | |
| gene | ESTs_{Incyte_PD:2556708} | 2556708 | 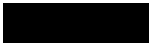 | |
| gene | KIAA0575_gene_product_{Incyte_PD:3094261} | 3094261 | 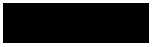 | |
| gene | ESTs_{Incyte_PD:1824332} | 1824332 | 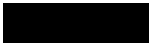 | |
| gene | Homo_sapiens_mRNA_chromosome_1_specific_transcript_KIAA0495_{Incyte_PD:1 963554} | 1963554 | 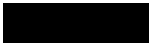 | |
| gene | ESTs_{Incyte_PD:2287483} | 2287483 | 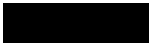 | |
| gene | ESTs_{Incyte_PD:3144018} | 3144018 | 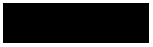 | |
| gene | ESTs_{Incyte_PD:2466668} | 2466668 | 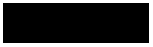 | |
| gene | ESTs_{Incyte_PD:2968970} | 2968970 | 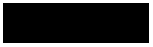 | |
| gene | ESTs_{Incyte_PD:2748370} | 2748370 | 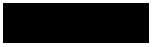 | |
| gene | ESTs,_Weakly_similar_to_!!!!_ALU_SUBFAMILY_SC_WARNING_ENTRY_!!!!_[H.sa piens]_{Incyte_PD:2243954} | 2243954 | 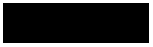 | |
| gene | transcription_factor_3_(E2A_immunoglobulin_enhancer_binding_factors_E12/E47)_{I ncyte_PD:640174} | 640174 | 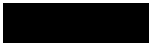 | |
| gene | Not_mapped_{Incyte_PD:3463469} | 3463469 | 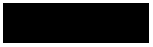 | |
| gene | ESTs_{Incyte_PD:201053) | 201053 | 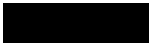 | |
| gene | ESTs_{Incyte_PD:865723) | 865723 | 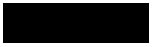 | |
| gene | adrenergic,_beta-2-,_receptor,_surface_{Incyte_PD:3200341) | 3200341 | 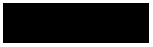 | |
| gene | solute_carrier_family_12_(sodium/chloride_transporters),_member_3_{Incyte_PD:35 35415) | 3535415 | 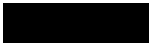 | |
| gene | ESTs_{Incyte_PD:4061967} | 4061967 | 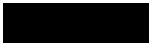 | |
| gene | ESTs,_Highly_similar_to_!!!!_ALU_SUBFAMILY_SP_WARNING_ENTRY_!!!!_[H.sap iens]_{Incyte_PD:966692} | 966692 | 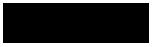 | |
| gene | Not_mapped_{Incyte_PD:2542313} | 2542313 | 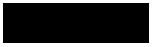 | |
| gene | ESTs_{Incyte_PD:1911371} | 1911371 | 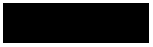 | |
| gene | ESTs,_Moderately_similar_to_!!!!_ALU_SUBFAMILY_J_WARNING_ENTRY_!!!!_[H. sapiens]_{Incyte_PD:2049587} | 2040587 | 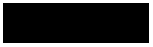 | |
| gene | ESTs_Moderately_similar_to_protein_serine/threonine_kinase_[H.sapiens]_{Incyte_PD:2789918} | 2789918 | 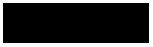 | |
| gene | ataxia_telangiectasia_mutated_(includes_complementation_groups_A,_C_and_D)_{I ncyte_PD:394665} | 394665 | 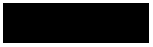 | |
| gene | Not_mapped_{Incyte_PD:2477854} | 2477854 | 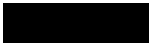 | |
| gene | Not_mapped_{Incyte_PD:2312442} | 2312442 | 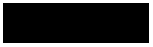 | |
| gene | WD_repeat_domain_2_{Incyte_PD:2210264} | 2210264 | 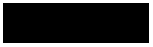 | |
| gene | Human_cytochrome_P450-IIB_(hIIB3)_mRNA,_complete_cds_{Incyte_PD:2796468} | 2796468 | 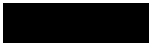 | |
| gene | dopa_decarboxylase_(aromatic_L-amino_acid_decarboxylase)_{Incyte_PD:2820985} | 2820985 | 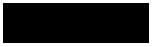 | important for growth of some carcinoid tumors |
| gene | potassium_channel,_subfamily_K,_member_1_(TWIK-1)_{Incyte_PD:1479255} | 1479255 | 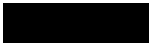 | |
| gene | ESTs_{Incyte_PD:2289901} | 2289901 | 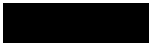 | |
| gene | ESTs_{Incyte_PD:1602726} | 1602726 | 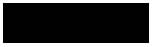 | |
| gene | primase,_polypeptide_1_(49kD)_{Incyte_PD:105121} | 105121 | 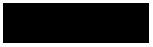 | |
| gene | ESTs_{Incyte_PD:2230152} | 2230152 | 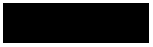 | |
| gene | midkine_(neurite_growth-promoting_factor_2)_{Incyte_PD :940845) | 940845 | 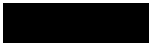 | cytokine expressed by numerous types of tumors. Our candidate in TCC |
| gene | ESTs_{Incyte_PD:2149237} | 2149237 | 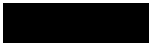 | |
| gene | ESTs_{Incyte_PD:1817969} | 1817969 | 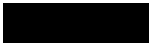 | |
| gene | ESTs_{Incyte_PD:1753445} | 1753445 | 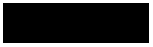 | |
| gene | ESTs_{Incyte_PD:2882960} | 2882960 | 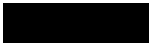 | |
| gene | ESTs_{Incyte_PD:213516} | 213516 | 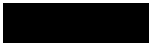 | |
| gene | ESTs_{Incyte_PD:2359527} | 2359527 | 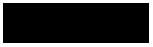 | |
| gene | ESTs,_Highly_similar_to_(defline_not_available_4589636)_[H.sapiens]_{Incyte_PD:2 499488} | 2499488 | 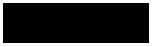 | |
| gene | ESTs_{Incyte_PD:2662127} | 2662127 | 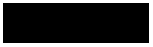 | |
| gene | ESTs_{Incyte_PD:2544622} | 2544622 | 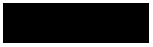 | |
| gene | Human_clone_23908_mRNA_sequence_Incyte_PD:2251851} | 2251851 | 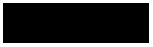 | |
| gene | phorbolin_(similar_to_apolipoprotein_B_mRNA_editing_protein)_{Incyte_PD:212573} | 212573 | 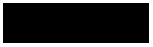 | |
| gene | platelet-activating_factor_receptor_{Incyte_PD:2966535} | 2966535 | 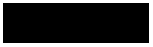 | |
| gene | EST_{Incyte_PD:2448338} | 2448338 | 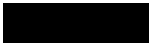 | |
| gene | ESTs_{Incyte_PD:2136337} | 2136337 | 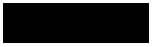 | |
| gene | ESTs_{Incyte_PD:4003220} | 4003220 | 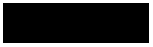 | |
| gene | ESTs_{Incyte_PD:2667106} | 2667106 | 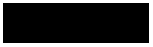 | |
| gene | ESTs,_Weakly_similar_to_!!!!_ALU_SUBFAMILY_J_WARNING_ENTRY_!!!!_[H.sapi ens]_{Incyte_PD:465591} | 465591 | 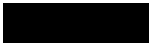 | |
| gene | ESTs_{Incyte_PD:2372541} | 2372541 | 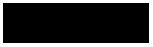 | |
| gene | E2F_transcription_factor_6_{Incyte_PD:14588} | 14588 | 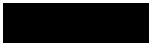 | |
| gene | ESTs_{Incyte_PD:2598965} | 2598965 | 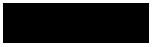 | |
| gene | ESTs,_Weakly_similar_to_!!!!_ALU_SUBFAMILY_SB_WARNING_ENTRY_!!!!_[H.sa piens]_{Incyte_PD:2075469} | 2075469 | 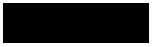 | |
| gene | ESTs_{Incyte_PD:2296027} | 2296027 | 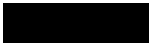 | |
| gene | Not_mapped_{Incyte_PD:2896792} | 2896792 | 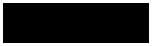 | |
| gene | ESTs,_Highly_similar_to_putative_RabS-interacting_protein_{clone_L1- 57}_[H.sapiens]_{Incyte_PD:1984130) | 1984130 | 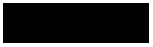 | |
| gene | ESTs_{Incyte_PD:1366043} | 1366043 | 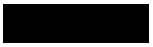 | |
| gene | ESTs,_Weakly_similar_to_!!!!_ALU_SUBFAMILY_J_WARNING_ENTRY_!!!!_[H.sapi ens]_{Incyte_PD:2133481} | 2133481 | 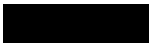 | |
| gene | ESTs_{Incyte_PD:1888670} | 1888670 | 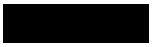 | |
| gene | ESTs_{Incyte_PD:1349433} | 1349433 | 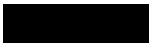 | |
| gene | ESTs,_Weakly_similar_to_putative_p150_[H.sapiens]_{Incyte_PD:2936403} | 2936403 | 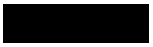 | |
| gene | ESTs_{Incyte_PD:414891} | 414891 | 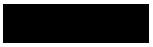 | |
| gene | ESTs_{Incyte_PD:2060416} | 2060416 | 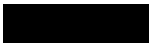 | |
| gene | ESTs_{Incyte_PD:3104921} | 3104921 | 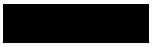 | |
| gene | ESTs_{Incyte_PD:57997} | 57997 | 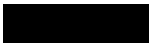 | |
| gene | Not_mapped_{Incyte_PD:1464613} | 1464613 | 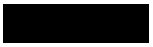 | |
| gene | ESTs_{Incyte_PD:1641775} | 1641775 | 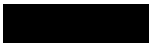 | |
| gene | Human_clone_23933_mRNA_sequence_{Incyte_PD:2286572} | 2286572 | 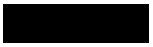 | |
| gene | amiloride_binding_protein_1_(amine_oxidase_(copper- containing)_{Incyte_PD:3676190} | 3676190 | 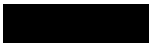 | |
| gene | Human_guanine_nucleotide_exchange_factor_mRNA,_complete_cds_{Incyte_PD:19 62190} | 1962190 | 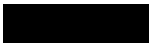 | |
| gene | ESTs_{Incyte_PD:2279230} | 2279230 | 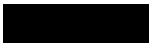 | |
| gene | ESTs_{Incyte_PD:3719865} | 3719865 | 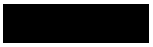 | |
| gene | preferentially_expressed_antigen_of_melanoma_{Incyte_PD:2007554} | 2007554 | 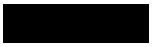 | melanoma antigen |
| gene | ESTs_{Incyte_PD:1302785} | 1302785 | 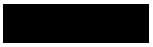 | |
| gene | Homo_sapiens_mRNA_from_chromosome_5q21- 22,_clone:FBR89_{Incyte_PD:2501484} | 2501484 | 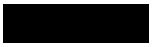 | |
| gene | pancreatic_lipase-related_pretein_1_{Incyte_PD:2084515} | 2084515 | 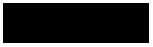 | |
| gene | ESTs_{Incyte_PD:2691093} | 2691093 | 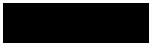 | |
| gene | ESTs,_Highly_similar_to_PP2C_[H.sapiens]_{Incyte_PD:2182353} | 2182353 | 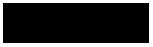 | |
| gene | surfactant,_pulmonary-associated_protein_B_{Incyte_PD:1988674} | 1988674 | 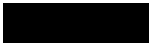 | |
| gene | ESTs,_Moderately_similar_to_!!!!_ALU_SUBFAMILY_J_WARNING_ENTRY_!!!!_[H. sapiens]_{Incyte_PD:520342} | 520342 | 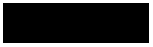 | |
| gene | ESTs,_Moderately_similar_to_!!!!_ALU_SUBFAMILY_SQ_WARNING_ENTRY_!!!!_[H.sapiens]_{Incyte_PD:1849453} | 1849453 | 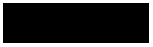 | |
| gene | ESTs,_Weakly_similar_to_(defline_not_available_4587207)_[H.sapiens]_{Incyte_PD: 2296344} | 2296344 | 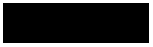 | |
| gene | ESTs_{Incyte_PD:2317034} | 2317034 | 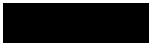 | |
| gene | ESTs,_Weakly_similar_to_!!!!_ALU_SUBFAMILY_SB_WARNING_ENTRY_!!!!_[H.sa piens]_{Incyte_PD:1638184} | 1638184 | 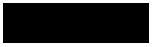 | |
| gene | ESTs_{Incyte_PD:1428856} | 1428856 | 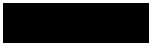 | |
| gene | ESTs_{Incyte_PD:3508727} | 3508727 | 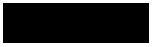 | |
| gene | ESTs,_Weakly_similar_to_!!!!_ALU_SUBFAMILY_J_WARNING_ENTRY_!!!!_[H.sapi ens]_{Incyte_PD:1431969} | 1431969 | 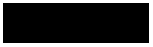 | |
| gene | Not_mapped_{Incyte_PD:1393855} | 1393855 | 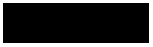 | |
| gene | ESTs_{Incyte_PD:2045755} | 2045755 | 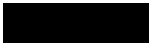 | |
| gene | ESTs_{Incyte_PD:1686727} | 1686727 | 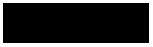 | |
| gene | ESTs_{Incyte_PD:2344817} | 2344817 | 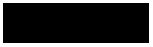 | |
| gene | KIAA0335_gene_product_{Incyte_PD:2308348} | 2308348 | 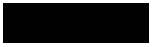 | |
| gene | ESTs_{Incyte_PD:1417114} | 1417114 | 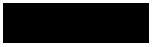 | |
| gene | ESTs_{Incyte_PD:2459069} | 2459069 | 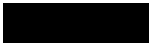 | |
| gene | putative_gene_product_{Incyte_PD:1300530} | 1300530 | 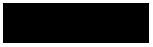 | |
| gene | breast_cancer_2_early_onset_{Incyte_PD:2468523} | 2468523 | 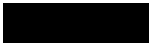 | |
| gene | nucleolar_autoantigen_(55kD)_similar_to_rat_synaptonemal_complex_protein_{Incyt e_PD:81490} | 81490 | 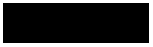 | |
| gene | kinesin_family_member_5C_{Incyte_PD:1596268} | 1596268 | 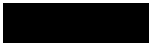 | |
| gene | ESTs,_Highly_similar_to_CDV-1R_protein_[M.musculus]_{Incyte_PD:1494531} | 1494531 | 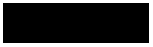 | |
| gene | ESTs_{Incyte_PD:2618859} | 2618859 | 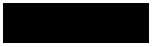 | |
| gene | ESTs_{Incyte_PD:2286816} | 2286816 | 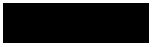 | |
| gene | activating_transcription_factor_4_(tax- responsive_enhancer_element_B67)_{Incyte_PD:2916261} | 2916261 | 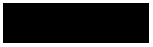 | |
| gene | ESTs_{Incyte_PD:1472206} | 1472206 | 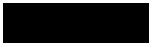 | |
| gene | desmocollin_2_{Incyte_PD:496003} | 496003 | 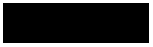 | |
| gene | Not_mapped_{Incyte_PD:341263} | 341263 | −2 | |
| gene | ESTs_{Incyte_PD:1440279} | 1440279 | −2 | |
| gene | differentiated_Embryo_Chondrocyte_expressed_gene_1_{Incyte_PD:1732479} | 1732479 | −2 | |
| gene | lectin,_galactoside-binding,_soluble,_3_(galectin_3)_{Incyte_PD:2921194} | 2921194 | −2 | |
| gene | calbindin_1,_(28kD)_{Incyte_PD:629769} | 629769 | −2 | |
| gene | serine_protease_inhibitor,_Kazal_type_1_{Incyte_PD:2373608} | 2373608 | −2 | |
| gene | golgi_SNAP_receptor_complex_member_2_{Incyte_PD:3279439} | 3279439 | −2 | |
| gene | Rho-associated_coiled-coil_containing_protein_kinase_1_{Incyte_PD:1351711} | 1351711 | −2 | |
| gene | plectin_1,_intermediate_filament_binding_protein,_500kD_{Incyte_PD:1907232} | 1907232 | −2 | |
| gene | ESTs,_Weakly_similar_to_DAP-1_beta_[H.sapiens]_{Incyte_PD:2902846} | 2902846 | −2 | |
| gene | actinin,_alpha_4_{Incyte_PD:1597330} | 1597330 | −2 | |
| gene | ubiquitin- | 1674422 | −2 | |
| | activating_enzyme_E1_(A1S9T_and_BN75_temperature_sensitivity_complementing) | | | |
| | _{Incyte_PD:1674422} | | | |
| gene | apoptosis_inhibitor_3_{Incyte_PD:2912879} | 2912879 | −2 | |
| gene | Human_mRNA_for_unknown_product,_partial_cds_{Incyte_PD:1402715} | 1402715 | −2 | |
| gene | ESTs,_Highly_similar_to_DIAMINE_ACETYLTRANSFERASE_[H.sapiens]_{Incyte_P | 63038 | −2 | |
| | D:63038} | | | |
| gene | ESTs₋{Incyte_PD:1912284} | 1912284 | −2 | |
| gene | Human_autoantigen_mRNA_complete_cds_{Incyte_PD:3374419} | 3374419 | −2 | |
| gene | ESTs_{Incyte_PD:565269} | 565269 | −2 | |
| gene | hect_(homologous_to_the_E6- | 4292515 | −2 | |
| | AP_(UBE3A)_carboxyl_terminus)_domain_and_RCC1_(CHC1)- | | | |
| | like_domain_(RLD)_1_{Incyte_PD:4292515} | | | |
| gene | phosphorylase,_glycogen;_muscle_(McArdle_syndrome,_glycogen_storage_disease | 2635943 | −2.0 | |
| | _type_V)_{Incyte_PD:2635943} | | | |
| gene | KIAA0336_gene_product_{Incyte_PD:3520448} | 3520448 | −2.1 | |
| gene | Homo_sapiens_SOX22_protein_(SOX22)_mRNA,_complete_cds_{Incyte_PD:28248 | 2824870 | −2.1 | |
| | 70} | | | |
| gene | Homo_sapiens_beta_III_spectrin_(SPTBN2)_mRNA,_partial_cds_{Incyte_PD:34322 | 3432208 | −2.1 | |
| | 08} | | | |
| gene | ESTs,_Weakly_similar_to_(defline_not_available_4929591)_[H.sapiens]_{Incyte_PD: | 3596244 | −2.1 | |
| | 3596244} | | | |
| gene | ubiquitin_specific_protease_5_(isopeptidase_T)_{Incyte_PD:2493777} | 2493777 | −2.1 | |
| gene | Homo_sapiens_mRNA_for_KIAA0841_protein,_partial_cds_{Incyte_PD:3971091} | 3971091 | −2.1 | |
| gene | laminin,_alpha_3_(nicein_(150kD),_kalinin_1_(165kD),_BM600_(150kD),_epilegrin)_{In | 1818527 | −2.1 | |
| | cyte_PD:1818527} | | | |
| gene | Human_mRNA_for_KIAA0185_gene,_partial_cds_{Incyte_PD:2503208} | 2503208 | −2.1 | |
| gene | desmoplakin_(DPI,_DPII)_{Incyte_PD:179929} | 179929 | −2.1 | |
| gene | cyclin-dependent_kinase_inhibitor_1A_(p21,_Cip1)_{Incyte_PD:1804548} | 1804548 | −2.1 | p21 |
| gene | alpha_thalassemia/mental_retardation_syndrome_X-linked_{Incyte_PD:41066291 | 4106629 | −2.2 | |
| gene | phosphatidylinositol-4-phosphate_5-kinase,_type_II_beta_{Incyte_PD:1315666} | 1315666 | −2.2 | |
| gene | ARP1_(actin- | 1841462 | −2.2 | |
| | related_protein_1,_yeast)_homolog_A_(centractin_alpha)_{Incyte_PD:1841462} | 1841462 | −2.2 | |
| gene | keratin_7_{Incyte_PD:16499591 | 1649959 | −2.2 | |
| gene | Homo_sapiens_mRNA_for_6-phosphofructo-2-kinase/fructose-2,_6- | 1302221 | −2.2 | |
| | bisphosphatase,_complete_cds_{Incyte_PD:1302221} | 1302221 | −2.2 | |
| gene | ESTs_{Incyte_PD:8552} | 8552 | −2.2 | |
| gene | vav_2_oncogene_{Incyte_PD:3744592} | 3744592 | −2.3 | |
| gene | epithelial_membrane_protein_1_{Incyte_PD:1624024} | 1624024 | −2.3 | |
| gene | ESTs_{Incyte_PD:2129939} | 2129939 | −2.3 | |
| gene | epidermal_growth_factor_receptor_(avian_erythroblastic_leukemia_viral_(v-erb- | 179598 | −2.3 | |
| | b)_oncogene_homolog)_{Incyte_PD:179598} | | | |
| gene | insulin-like_growth_factor_1_receptor_{Incyte_PD:2597262} | 2597262 | −2.3 | |
| gene | protein_kinase_C_substrate_80K-H_{Incyte_PD:1723971} | 1723971 | −2.3 | |
| gene | guanylate_binding_protein_2,_interferon-inducible_{Incyte_PD:1610993} | 1610993 | −2.3 | |
| gene | interferon,_gamma-inducible_protein_16_{Incyte_PD:2508261} | 2508261 | −2.3 | |
| gene | N-myc_downstream_regulated_{Incyte_PD:2055569} | 2055569 | −2.3 | |
| gene | ESTs_{Incyte_PD:3971258} | 3971258 | −2.4 | |
| gene | Homo_sapiens_transcriptional_enhancer_factor_(TEF1)_DNA,_complete_CDS_{Incy | 2957175 | −2.4 | |
| | te_PD:2957175} | | | |
| gene | protein_tyrosine_phosphatase,_receptor_type,_J_{Incyte_PD:3818113} | 3818113 | −2.4 | |
| gene | Human_cleavage_signal_1_protein_mRNA,_complete_cds_{Incyte_PD:2054053} | 2054053 | −2.4 | |
| gene | ESTs_{Incyte_PD:187276} | 187276 | −2.4 | |
| gene | hypothetical_protein_of_unknown_function_{Incyte_PD:1402462} | 1402462 | −2.5 | |
| gene | Human_mRNA_for_KIAA0066_gene,_partial_cds_{Incyte_PD:3042637} | 3042637 | −2.5 | |
| gene | BMX_non-receptor_tyrosine_kinase_{Incyte_PD:1655995} | 1655995 | −2.5 | important for NE differentiation |
| gene | nel_(chicken)-like_2_{Incyte_PD:2285502} | 2285502 | −2.5 | |
| gene | homolog_of_Drosophila_past_{Incyte_PD:1336738} | 1336738 | −2.5 | |
| gene | ESTs_{Incyte_PD:1382947} | 1382947 | −2.5 | |
| gene | ESTs_{Incyte_PD:1967531} | 1967531 | −2.5 | |
| gene | ESTs_{Incyte_PD:2369022} | 2369022 | −2.5 | |
| gene | cannabinoid_receptor_1_(brain)_{Incyte_PD:112853} | 112853 | −2.6 | antagonizes NGF stimulation of trk. Trk's are targets in prostate |
| | | | | cancer treatemnt, tumors produce NGF |
| gene | keratin,_hair,_basic,_1_{Incyte_PD:1919158} | 1919158 | −2.6 | |
| gene | S100_calcium- | 1222317 | −2.6 | |
| | binding_protein_A4_calcium_protein,_calvasculin,_metastasin,_murine_placental_h | | | |
| | omolog)_{Incyte_PD:1222317} | | | |
| gene | fibroblast_activation_protein,_alpha_{Incyte_PD:2483605} | 2483605 | −2.6 | |
| gene | Homo_sapiens_KIAA0421_mRNA,_partial_cds_{Incyte_PDL4253663} | 4253663 | −2.6 | |
| gene | Not_mapped_{Incyte_PD:2204871} | 2204871 | −2.7 | |
| gene | lamin_B2_{Incyte_PD:2414632} | 2414632 | −2.7 | |
| gene | KIAA0138_gene_product_{Incyte_PD:1731569} | 1731569 | −2.7 | |
| gene | H.sapiens_mRNA_for_protein-tyrosine-phosphatase_D1_{Incyte_PD:2605804} | 2605804 | −2.7 | |
| gene | axonal_transport_of_synaptic_vesicles_{Incyte_PD:3856893} | 3856893 | −2.7 | |
| gene | Homo_sapiens_mRNA;_cDNA_DKFZp586F0219_(from_clone_DKFZp586F0219)_{I | 220566 | −2.8 | |
| | ncyte_PD:220566} | | | |
| gene | nuclear_factor_I/C_(CCAAT-binding_transcription_factor)_{Incyte_PD:1670221} | 1670221 | −2.8 | |
| gene | ESTs_{Incyte_PD:1540157} | 1540157 | −2.8 | |
| gene | 5T4_oncofetal_trophoblast_glycoprotein_{Incyte_PD:1283532} | 1283532 | −2.8 | |
| gene | glucose-6- | 4287327 | −2.8 | |
| | phosphatase,_catalytic_(glycogen_storage_disease_type_I,_von_Gierke_disease)_{I | | | |
| | ncyte_PD:4287327} | | | |
| gene | interleukin_6_signal_transducer_(gp130, oncostatin_M_receptor)_{Incyte_PD:21723 | 2172334 | −2.8 | growth suppresor |
| | 34} | | | |
| gene | hydroxyprostaglandin_dehydrogenase_15-(NAD)_{Incyte_PD:1578941} | 1578941 | −2.8 | |
| gene | v-myb_avian_myeloblastpsis_viral_oncogene_homolog_like_2_{Incyte_PD:494905} | 494905 | −2.9 | |
| gene | Homo_sapiens_Nedd-4-like_ubiquitin- | 869818 | −2.9 | |
| | protein_ligase_WWP2_mRNA_complete_cds_{Incyte_PD:869818} | | | |
| gene | UDP-Gal:betaGlcNAc_beta_1,4- | 1736926 | −3 | |
| | _galactosyltransferase,_polypeptide_1_{Incyte_PD:1736926} | | | |
| gene | Human_mRNA_for_KIAA0220_gene,_partial_cds_{Incyte_PD:2657738} | 2657738 | −3 | |
| gene | splicing_factor,_arginine/serine-rich_6_{Incyte_PD:1728574} | 1728574 | −3 | |
| gene | interleukin_enhancer_binding_factor_3,_90kD_{Incyte_PD:1674263} | 1674263 | −3.1 | |
| gene | ESTs_{Incyte_PD:1635864} | 1635864 | −3.2 | |
| gene | regulator_of_G-protein_signalling_4_{Incyte_PD:617517} | 617517 | −3.3 | |
| gene | eukaryotic_translation_initiation_factor_4_gamma,_1_{Incyte_PD:1965695} | 1965695 | −3.4 | |
| gene | REMOVED_FROM_DATABASE_{Incyte_PD:85246} | 85246 | −3.4 | |
| gene | zinc_finger_protein_162_{Incyte_PD:2655068} | 2655068 | −3.5 | |
| gene | Homo_sapiens_TTF- | 4251662 | −3.7 | |
| | I_interacting_peptide_5_mRNA,_partial_cds_{Incyte_PD:4251662} | 14251662 | −3.7 | |
| gene | Homo_sapiens_ataxin-2- | 1712724 | −3.7 | |
| | like_protein_A2LP_(A2LG)_mRNA,_complete_cds_{Incyte_PD:1712724} | | | |
| gene | quiescin_Q6_{Incyte_PD:1854220} | 1854220 | −3.8 | |
| gene | Human_transporter_protein_(g17)_mRNA,_complete_cds_{Incyte_PD:86661} | 86661 | −3.9 | |
| gene | nuclear_mitotic_apparatus_protein_1_{Incyte_PD:2700234} | 2700234 | −3.9 | |
| gene | yotiao_{Incyte_PD:3868809} | 3868809 | −4.1 | |
| gene | myosin,_heavy_polypeptide_11,_smooth_muscle_{Incyte_PD:1866751} | 1866751 | −4.1 | |
| gene | cut_(Drosophila)-like_1_(CCAAT_displacement_{—protein)}_{Incyte_PD:2372648} | 2372648 | −4.1 | |
| gene | filamin_A_alpha_(actin-binding_protein-280)_{Incyte_PD:1708528} | 1708528 | −4.2 | |
| gene | nucleoporin_98kD_{Incyte_PD:1611993} | 1611993 | −4.2 | |
| gene | ESTs,_Highly_similar_to_(defline_not_available_3986200)_[H.sapiens]_{Incyte_PD:1 | 1554358 | −4.4 | |
| | 554358} | | | |
| gene | Human_mRNA_for_KIAA0194_gene,_partial_cds{Incyte_PD:1429306} | 1429306 | −4.5 | |
| gene | microtubule-associated_protein_4_{Incyte_PD:2992994} | 2992994 | −4.6 | |
| gene | US_snRNP- | 3616229 | −4.7 | |
| | specific_protein_(220_kD),_ortholog_of_S._cerevisiae_Prp8p_{Incyte_PD:3616229} | 3616229 | −4.7 | |
| gene | ser- | 1602261 | −4.8 | |
| | Thr_protein_kinase_related_to_the_myotonic_dystrophy_protein kinase_{Incyte_PD: | | | |
| | 1602261} | | | |
| gene | hect_domain_and_RLD_2_{Incyte_PD:2739109} | 2739109 | −4.9 | |
| gene | KIAA0018_gene_product_{Incyte_PD:2962167} | 2962167 | −4.9 | sedalin-1 |
| gene | laminin,_gamma_1_(formerly_LAMB2)_{Incyte_PD:1599344} | 1599344 | −4.9 | |
| gene | Not_mapped_{Incyte_PD:3427594} | 3427594 | −5.5 | |
| gene | protein_phosphatase_1,_regulatory_subunit_10_{Incyte_PD:2314555} | 2314555 | −5.7 | |
| gene | general_transcription_factor_IIIC,_polypeptide_1_(alpha_subunit,_220kD_)_{Incyte_ | 486304 | −6.7 | |
| | PD:486304} | | | |
| gene | ESTs,_Highly_similar_to_Ran/TC4- | 3030988 | −7 | |
| | binding_nucleopore_protein_[H.sapiens]_{Incyte_PD:3030988} | | | |
| gene | Homo_sapiens_mRNA_for_KIAA0911_protein,_complete_cds_{Incyte_PD:2578710} | 2578710 | −7.4 | |
| gene | valyl-tRNA_synthetase_1_{Incyte_PD:1829709} | 1829709 | −28 | |

BREAST

| 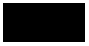 | spl | panc | liv | stom |
|---|---|---|---|---|
| Description | PL-4B_BDEnor | PL-2B_BDEnor | PL-6B_BDEnor | PL-8B_BDEnor |
| 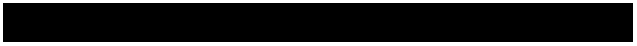 | −1.2 | −1.3 | 1.1 | −1.1 |
| 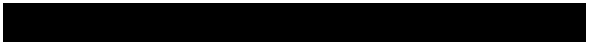 | −1.3 | −1.0 | 1.1 | −1.1 |
| 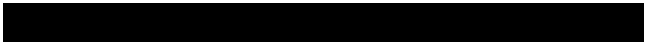 | −1.4 | −1.7 | −1.2 | −1.4 |
| 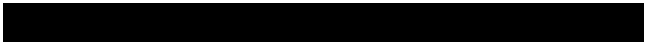 | 1.5 | 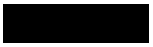 | −1.4 | 1.1 |
| 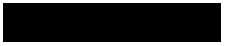 | 1.1 | −1.4 | −1.4 | 1.3 |
| 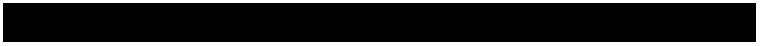 | 1.2 | −1.6 | −1.3 | 1.5 |
| AA220024_trophoblast_specific_protein | 1.5 | −2.0 | −1.2 | 1.5 |
| AI549624_RIKEN_cDNA_0610041E09_gene | 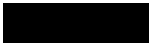 | 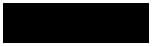 | −1.3 | 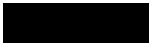 |
| AI1326499_epidermal_growth_factor | 1.3 | −1.4 | −1.2 | 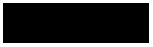 |
| AI1876997_carboxyl_ester_lipase | 1.5 | 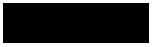 | −1.4 | −2.3 |
| AA469630_choline_kinase | 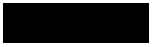 | 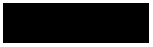 | −1.4 | 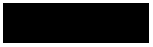 |
| W14224_N-myc_downstream_regulated_1 | 1.1 | 1.0 | 1.0 | 1.1 |
| AA529824_fatty_acid_binding_protein_2,_intestinal | −1.5 | −1.5 | −1.7 | 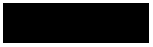 |
| AI527496_Mus_musculus_mRNA,_com- plete_cds,_similar_to_L-amino_acid_oxidase | 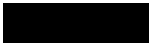 | −1.2 | −1.5 | −1.2 |
| AI447967_ESTs | 1.1 | 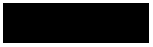 | 1.0557+ | 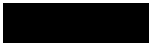 |
| AA588976_ESTs | 1.1 | 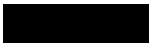 | −1.5 | 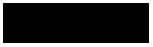 |
| AA080175_claudin_8 | −1.0 | 1.1 | 1.3 | 1.1 |
| AI481982_EST | 1.5 | 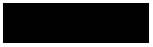 | 1.0 | 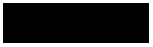 |
| AI553220_ESTs | 1.47814* | −1.0 | 1.3 | 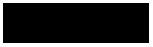 |
| AA792499_ankyrin-like_repeat_protein | 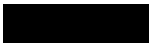 | −1.8 | −1.5 | 1.1 |
| AI549654_programmed_cell_death_10 | 1.24627+ | 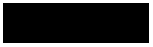 | −1.0038+ | 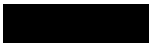 |
| AA210488_ESTs | 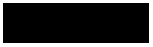 | 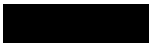 | −1.1 | 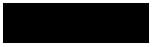 |
| AI614454_reduced_in_osteosclerosis_transporter | 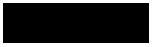 | −1.1 | −1.8 | 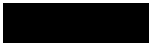 |
| AA288467_glycosylation_dependent_cell_adhesion_molecule_1 | −1.0 | −1.8 | 1.2 | 1.2 |
| AA290390_cell_death-inducing_DNA_frag- | 1.2 | −1.5 | 1.4 | 1.2 |
| mentation_factor,_alpha_subunit-like_effector_8 | | | | |
| AA445667_RIKEN_cDNA_4632401C08_gene | 1.3 | −1.2 | −2.0 | 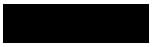 |
| AA623060_RIKEN_cDNA_2410124L17_gene | 1.5 | 1.5 | 1.1 | 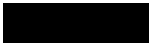 |
| AA617112_ESTs | 1.4 | 1.4 | −1.2 | 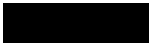 |
| AA064586_testis_specific_gene_A2 | 1.1 | −1.5 | 1.2 | 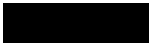 |
| AA073925_ESTs | 1.3 | 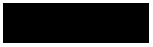 | −1.4 | 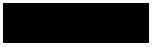 |
| AI180945_ESTs,_Moderately_similar_to_S- | 1.4 | −1.6 | 1.5 | 1.5 |
| ACYL_FATTY_ACID_SYNTHASE_THIO- | | | | |
| ESTERASE,_Medium_CHAIN_[R.norvegicus] | | | | |
| AI892299_RIKEN_cDNA_5830499B15_gene | 1.0 | −1.3 | 1.1 | 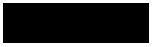 |
| AI466648_RIKEN_cDNA_5730427C23_gene | −1.1 | −1.5 | −1.2 | 1.2 |
| AI154946_arginase_type_II | −1.1 | 1.0 | −1.2 | −1.1 |
| W12985_annexin_A10 | 1.5 | −1.7 | −1.1 | −2.3 |
| AW209707_hemoglobin_Z,_beta-like_embryonic_chain | −1.5 | −1.5 | 1.1 | −1.8 |
| AI593096_ESTs,_Weakly_similar_to_NTC1_MOUSE_NEURO- | −1.3 | −1.3 | −1.0 | 1.1 |
| GENIC_LOCUS_NOTCH_HOMOLOG_PROTEIN_1_PREC | | | | |
| URSOR_[M.musculus] | | | | |
| AA725961_ESTs | −1.25704+ | −1.37307+ | −1.03755+ | −1.33228+ |
| AA607044_RIKEN_cDNA_2700099C19_gene | −1.4 | −1.3 | −1.5 | −2.2 |
| AA611085_glypican_1 | 1.2665+ | −1.05915+ | 1.19353+ | −1.10085+ |
| AA675111_RIKEN_cDNA_1810007A24_gene | 1.4 | 1.3 | −1.0 | −2.1 |
| AA066763_hemoglobin,_beta_adult_major_chain | 1.3 | −1.2 | 1.4 | −4.0 |
| AA106071_hemoglobin,_beta_adult_major_chain | 1.3 | −1.4 | 1.2 | −3.0 |
| AA840565_RIKEN_cDNA_1300019P08_gene | −1.2 | −1.2 | −1.1 | −1.1 |
| AA718468_ESTs | −1.16834+ | −1.40655+ | 1.07363+ | −1.2 |
| AI536309_RIKEN_cDNA_2210410L06_gene | −1.8 | −1.8 | −1.4 | −2.1 |
| AI158547_ESTs | −1.0 | −1.24926+ | −1.05647+ | −1.32972+ |
| W41372_uncoupling_protein,_mitochondrial | 1.2 | −1.1 | −1.0 | −1.1 |
| AA182334_immunoglobulin_heavy_chain_1_(serum_IgG2a) | 1.0 | −1.16626+ | −1.0 | 1.1 |
| W33918_serine_(or_cystein)_proteinase_inhibitor,_clade_F_(alpha- 2_antiplasmin,_pigment_epithelium_derived_factor),_member_2 | 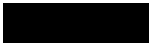 | −1.1 | 1.5 | −1.1 |
| AA967824_arginine_vasopressin | 1.4 | −1.6 | −1.2 | −1.1 |
| AA066835_corticosteroid_binding_globulin | 1.0 | 1.2 | 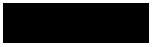 | −1.1 |
| AA138265_RIKEN_cDNA_2010309G21_gene | −1.1 | −2.2 | −1.5 | −1.2 |
| AA152940_immunoglobulin_kappa_chain_variable_28_(V28) | 1.3 | −1.6 | −1.5 | −1.0 |
| AA822106_ESTs,_Weakly_similar_to_CO8A_HUMAN_COM- PLEMENT_COMPONENT_C8_ALPHA_CHAIN_PRECURSO R_[H.sapiens] | 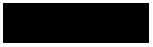 | −1.7 | 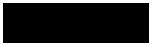 | −1.0 |
| AW496194_immuno- | 1.4 | −1.2 | −1.4 | −1.1 |
| globulin_kappa_chain_chain_variable_20_(V20_family) | | | | |
| AI386058_serum_albumin_variant | 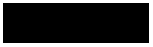 | −1.7 | 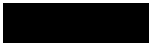 | 1.1 |
| AI158823_immunoglobulin_heavy_chain_3_(serum_IgG2b) | −1.0 | −1.6 | 1.0 | 1.5 |

BREAST

| 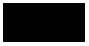 | int | col | br | lung |
|---|---|---|---|---|
| Description | PL-3B_BDEnor | PL-5B_BDEnor | PL-1B_BDEnor | PL-7B_BDEnor |
| 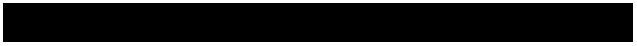 | 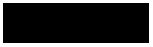 | 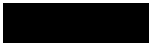 | −1.5 | −1.9 |
| 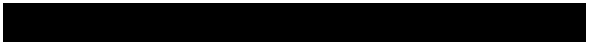 | −1.1 | 1.17304+˜ | 1.2 | −1.1 |
| 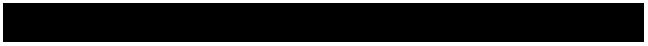 | −1.2 | 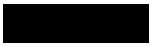 | −2.5 | −1.0 |
| 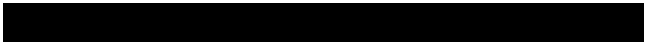 | 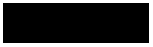 | −1.0 | −1.8 | −1.2 |
| 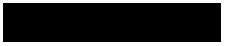 | 1.3 | 1.2 | −1.6 | −1.3 |
| 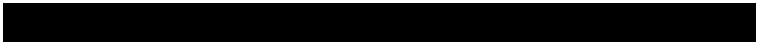 | 1.3 | 1.27823˜ | −2.2 | −1.0 |
| AA220024_trophoblast_specific_protein | 1.1 | −1.0 | −2.2 | −1.2 |
| AI549624_RIKEN_cDNA_0610041E09_gene | 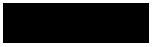 | −1.01549+˜ | 1.1 | 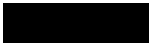 |
| AI1326499_epidermal_growth_factor | −1.1 | −1.4 | −1.4 | 1.5 |
| AI1876997_carboxyl_ester_lipase | −8.9 | −1.9 | −1.3 | −1.4 |
| AA469630_choline_kinase | 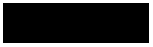 | 1.31548+˜ | 1.3 | 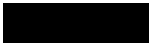 |
| W14224_N-myc_downstream_regulated_1 | 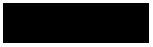 | −1.0 | 1.2 | 1.1 |
| AA529824_fatty_acid_binding_protein_2,_intestinal | −1.1 | 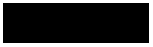 | −2.3 | 1.0 |
| AI527496_Mus_musculus_mRNA,_com- plete_cds,_similar_to_L-amino_acid_oxidase | −1.1 | −1019867˜ | −1.6 | 1.1 |
| AI447967_ESTs | 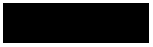 | −1.08065+˜ | 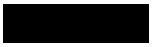 | 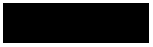 |
| AA588976_ESTs | 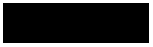 | 1.14361+˜ | 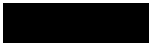 | 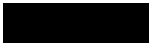 |
| AA080175_claudin_8 | 1.1 | −1.00278˜ | 1.2 | −1.2 |
| AI481982_EST | 1.4283* | −1.60668+˜ | 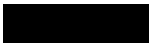 | 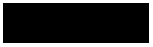 |
| AI553220_ESTs | 1.6813* | −1.30242˜ | −1.1 | 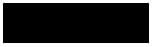 |
| AA792499_ankyrin-like_repeat_protein | 1.2 | 1.05661˜ | −1.4 | 1.1 |
| AI549654_programmed_cell_death_10 | 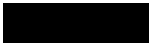 | 1.10261+˜ | 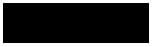 | 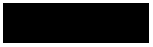 |
| AA210488_ESTs | 1.5 | 1.2483+˜ | 1.2 | 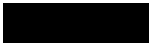 |
| AI614454_reduced_in_osteosclerosis_transporter | 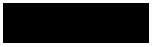 | 1.11125˜ | 1.3 | 1.2 |
| AA288467_glycosylation_dependent_cell_adhesion_molecule_1 | −1.1 | 1.2 | −2.1 | −1.3 |
| AA290390_cell_death-inducing_DNA_frag- | 1.3 | 1.1 | −1.5 | −1.6 |
| mentation_factor,_alpha_subunit-like_effector_8 | | | | |
| AA445667_RIKEN_cDNA_4632401C08_gene | −1.0 | 1.1 | 1.3 | −1.2 |
| AA623060_RIKEN_cDNA_2410124L17_gene | 1.36143* | −1.3 | 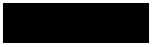 | 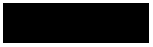 |
| AA617112_ESTs | 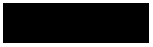 | −1.01971+˜ | 1.3 | 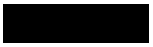 |
| AA064586_testis_specific_gene_A2 | 1.3 | 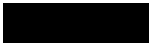 | −1.6 | −1.0 |
| AA073925_ESTs | 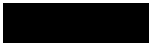 | −1.27077˜ | 1.1 | 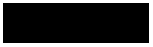 |
| AI180945_ESTs,_Moderately_similar_to_S- | 1.1 | −1.11723˜ | −1.8 | −1.0 |
| ACYL_FATTY_ACID_SYNTHASE_THIO- | | | | |
| ESTERASE,_Medium_CHAIN_[R.norvegicus] | | | | |
| AI892299_RIKEN_cDNA_5830499B15_gene | 1.3 | −2.1 | −1.8 | −1.0 |
| AI466648_RIKEN_cDNA_5730427C23_gene | −1.0 | 1.20303˜ | −1.1 | −1.2 |
| AI154946_arginase_type_II | −1.2 | 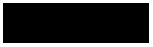 | −1.6 | 1.1 |
| W12985_annexin_A10 | −8.5 | −1.03567˜ | −1.3 | −1.1 |
| AW209707_hemoglobin_Z,_beta-like_embryonic_chain | −1.4 | −1.80487˜ | 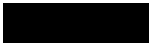 | −1.2 |
| AI593096_ESTs,_Weakly_similar_to_NTC1_MOUSE_NEURO- GENIC_LOCUS_NOTCH_HOMOLOG_PROTEIN_1_PREC URSOR_[M.musculus] | 1.2 | 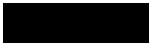 | −1.2 | −1.2 |
| AA725961_ESTs | −1.0 | 1.4912+˜ | −1.2 | −1.178+ |
| AA607044_RIKEN_cDNA_2700099C19_gene | −1.0 | −1.4 | −1.1 | −1.4 |
| AA611085_glypican_1 | 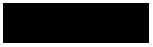 | −1.26963+ | 1.20291+ | 1.04301+ |
| AA675111_RIKEN_cDNA_1810007A24_gene | −1.5 | 1.0 | −1.4 | −2.2 |
| AA066763_hemoglobin,_beta_adult_major_chain | −4.2 | 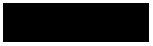 | −1.1 | −1.3 |
| AA106071_hemoglobin,_beta_adult_major_chain | −4.3 | 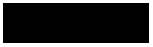 | 1.1 | −1.3 |
| AA840565_RIKEN_cDNA_1300019P08_gene | 1.20536˜ | −1.5 | −1.4 | −1.1 |
| AA718468_ESTs | 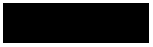 | 1.07881+ | −1.09891+ | −1.3 |
| AI536309_RIKEN_cDNA_2210410L06_gene | 1.1 | −1.5 | −2.0 | −1.6 |
| AI158547_ESTs | −1.49596+˜ | −1.5 | 1.33285+ | −1.1477+ |
| W41372_uncoupling_protein,_mitochondrial | 1.05988+˜ | 1.1 | −1.8 | 1.1 |
| AA182334_immunoglobulin_heavy_chain_1_(serum_IgG2a) | −1.84425˜ | −1.14317+ | 1.1 | −1.1 |
| W33918_serine_(or_cystein)_proteinase_inhibitor,_clade_F_(alpha- | −1.36639˜ | 1.2 | −3.1 | 1.1 |
| 2_antiplasmin,_pigment_epithelium_derived_factor_),_member_2 | | | | |
| AA967824_arginine_vasopressin | −1.9 | −2.7 | −2.0 | −1.4 |
| AA066835_corticosteroid_binding_globulin | 1.2 | 1.4 | −2.4 | −2.3 |
| AA138265_RIKEN_cDNA_2010309G21_gene | −1.0 | −1.9 | −1.1 | 1.1 |
| AA152940_immunoglobulin_kappa_chain_variable_28_(V28) | −2.1 | −3.1 | −2.2 | −1.3 |
| AA822106_ESTs,_Weakly_similar_to_CO8A_HUMAN_COM- PLEMENT_COMPONENT_C8_ALPHA_CHAIN_PRECURSO R_[H.sapiens] | 1.1 | 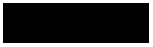 | −1.8 | −20.2 |
| AW496194_immuno- | −1.9 | −2.9 | −2.6 | −1.6 |
| globulin_kappa_chain_chain_variable_20_(V20_family) | | | | |
| AI386058_serum_albumin_variant | −1.1 | 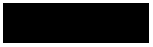 | −1.2 | −23.1 |
| AI158823_immunoglobulin_heavy_chain_3_(serum_IgG2b) | 1.5 | −2.52501˜ | 1.2 | 1.2 |

BREAST

| 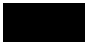 | blad | kidn | pl/ut | 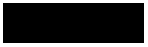 |
|---|---|---|---|---|
| Description | PL-9B_BDEnor | PL-10B_BDEnor | PL-11B_BDEnor | PL-12B_BDEnor |
| 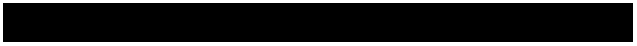 | −1.2 | 1.2 | −1.3 | 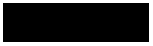 |
| 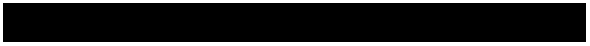 | −1.0 | −1.1 | −1.1 | 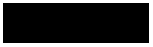 |
| 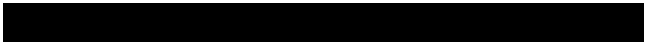 | −1.3 | −1.3 | −1.5 | 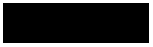 |
| 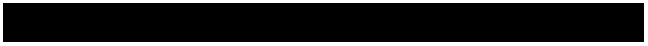 | −9.2 | −1.0 | 1.0 | 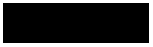 |
| 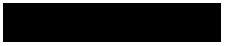 | 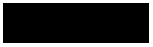 | 1.2 | 1.3 | 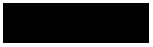 |
| 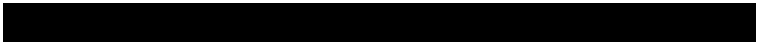 | −1.1 | 1.0 | 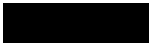 | 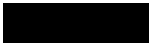 |
| AA220024_trophoblast_specific_protein | 1.4 | 1.1 | 1.2 | 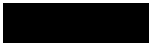 |
| AI549624_RIKEN_cDNA_0610041E09_gene | 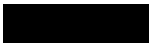 | 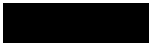 | 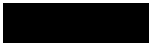 | 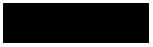 |
| AI1326499_epidermal_growth_factor | 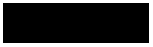 | 1.3 | 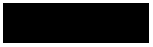 | 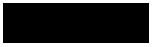 |
| AI1876997_carboxyl_ester_lipase | −1.1 | 1.1 | 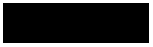 | 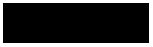 |
| AA469630_choline_kinase | 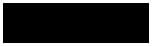 | 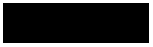 | 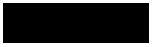 | 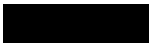 |
| W14224_N-myc_downstream_regulated_1 | 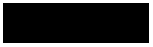 | 1.0 | 1.3 | 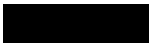 |
| AA529824_fatty_acid_binding_protein_2,_intestinal | 1.4 | 1.1 | 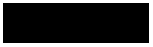 | 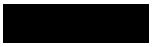 |
| AI527496_Mus_musculus_mRNA,_com- plete_cds,_similar_to_L-amino_acid_oxidase | −1.1 | −1.1 | 1.3 | 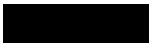 |
| AI447967_ESTs | 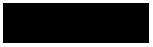 | 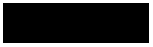 | 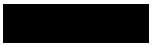 | 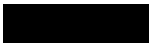 |
| AA588976_ESTs | 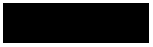 | 1.4 | 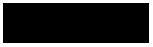 | 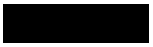 |
| AA080175_claudin_8 | 1.3 | −1.1 | −1.2 | 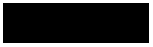 |
| AI481982_EST | 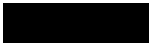 | 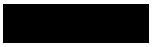 | 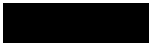 | 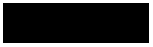 |
| AI553220_ESTs | 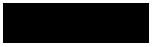 | 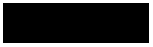 | 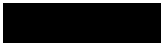 | 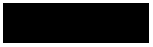 |
| AA792499_ankyrin-like_repeat_protein | −1.2 | 1.4 | 1.2 | 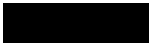 |
| AI549654_programmed_cell_death_10 | 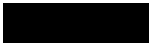 | 1.3 | 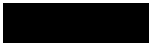 | 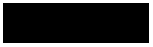 |
| AA210488_ESTs | 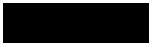 | 1.4 | 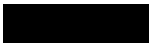 | 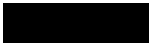 |
| AI614454_reduced_in_osteosclerosis_transporter | 1.2 | 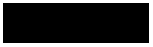 | 1.5 | 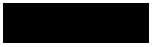 |
| AA288467_glycosylation_dependent_cell_adhesion_molecule_1 | −1.0 | −1.1 | 1.2 | 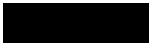 |
| AA290390_cell_death-inducing_DNA_frag- | 1.3 | 1.2 | 1.2 | 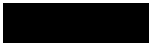 |
| mentation_factor,_alpha_subunit-like_effector_8 | | | | |
| AA445667_RIKEN_cDNA_4632401C08_gene | 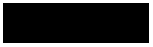 | 1.4 | 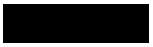 | 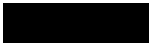 |
| AA623060_RIKEN_cDNA_2410124L17_gene | 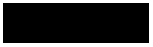 | 1.4 | 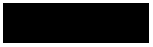 | 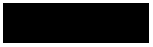 |
| AA617112_ESTs | 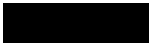 | 1.3 | 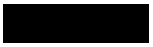 | 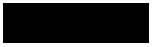 |
| AA064586_testis_specific_gene_A2 | 1.3 | 1.1 | 1.4 | 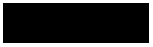 |
| AA073925_ESTs | 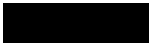 | 1.2 | 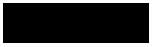 | 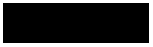 |
| AI180945_ESTs,_Moderately_similar_to_S- | 1.3 | 1.1 | 1.2 | 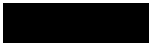 |
| ACYL_FATTY_ACID_SYNTHASE_THIO- | | | | |
| ESTERASE,_Medium_CHAIN_[R.norvegicus] | | | | |
| AI892299_RIKEN_cDNA_5830499B15_gene | 1.5 | 1.2 | 1.2 | 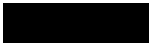 |
| AI466648_RIKEN_cDNA_5730427C23_gene | 1.2 | 1.2 | 1.2 | 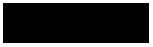 |
| AI154946_arginase_type_II | 1.0 | 1.2 | 1.1 | 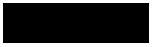 |
| W12985_annexin_A10 | 1.1 | −1.0 | 1.0 | −1.9 |
| AW209707_hemoglobin_Z,_beta-like_embryonic_chain | −1.4 | 1.1 | −2.7 | −1.9 |
| AI593096_ESTs,_Weakly_similar_to_NTC1_MOUSE_NEURO- | −1.5 | −1.3 | −1.7 | −1.9 |
| GENIC_LOCUS_NOTCH_HOMOLOG_PROTEIN_1_PREC | | | | |
| URSOR_[M.musculus] | | | | |
| AA725961_ESTs | −1.3 | −1.2 | −1.1 | −1.9 |
| AA607044_RIKEN_cDNA_2700099C19_gene | −1.4 | −1.3 | −1.3 | −1.9 |
| AA611085_glypican_1 | 1.04301+ | −1.3 | −1.1 | −1.9 |
| AA675111_RIKEN_cDNA_1810007A24_gene | −2.2 | −1.2 | −1.2 | −2.0 |
| AA066763_hemoglobin,_beta_adult_major_chain | −1.3 | 1.5 | −1.8 | −2.0 |
| AA106071_hemoglobin,_beta_adult_major_chain | −1.3 | 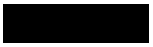 | −1.7 | −2.0 |
| AA840565_RIKEN_cDNA_1300019P08_gene | −1.1 | −1.3 | −1.2 | −2.1 |
| AA718468_ESTs | −1.3 | −1.18257+ | −1.3 | −2.1 |
| AI536309_RIKEN_cDNA_2210410L06_gene | −1.6 | −2.1 | −1.5 | −2.1 |
| AI158547_ESTs | −1.1477+ | −1.3 | 1.2 | −2.2 |
| W41372_uncoupling_protein,_mitochondrial | 1.1 | −1.1 | −1.1 | −2.2 |
| AA182334_immunoglobulin_heavy_chain_1_(serum_IgG2a) | −1.1 | 1.2 | 1.1 | −2.2 |
| W33918_serine_(or_cystein)_proteinase_inhibitor,_clade_F_(alpha- | 1.1 | 1.4 | −1.6 | −2.2 |
| 2_antiplasmin,_pigment_epithelium_derived_factor),_member_2 | | | | |
| AA967824_arginine_vasopressin | −1.3 | 1.4 | −1.2 | −2.3 |
| AA066835_corticosteroid_binding_globulin | 1.1 | −1.0 | −1.3 | −2.4 |
| AA138265_RIKEN_cDNA_2010309G21_gene | −1.3 | −1.0 | −1.0 | −2.4 |
| AA152940_immunoglobulin_kappa_chain_variable_28_(V28) | −1.3 | 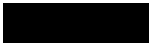 | −1.1 | −2.5 |
| AA822106_ESTs,_Weakly_similar_to_CO8A_HUMAN_COM- | −1.5 | −1.3 | −1.4 | −2.7 |
| PLEMENT_COMPONENT_C8_ALPHA_CHAIN_PRECURSO | | | | |
| R_[H.sapiens] | | | | |
| AW496194_immuno- | −1.0 | 1.4 | −1.0 | −2.7 |
| globulin_kappa_chain_chain_variable_20_(V20_family) | | | | |
| AI386058_serum_albumin_variant | −1.4 | −1.5 | −1.3 | −2.9 |
| AI158823_immunoglobulin_heavy_chain_3_(serum_IgG2b) | 1.4 | −1.0 | 1.2 | −3.4 |

PLACENTA/UTERUS

| 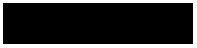 | spl | panc | liv | stom |
|---|---|---|---|---|
| Description | PL-4B_BDEnor | PL-2B_BDEnor | PL-6B_BDEnor | PL-8B_BDEnor |
| 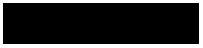 | 1.5 | 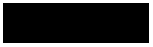 | 1.0 | 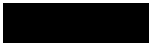 |
| 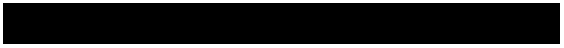 | 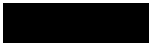 | 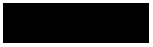 | −1.3 | 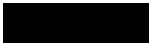 |
| AA270885_parvalbumin | −1.1 | −1.4 | −1.4 | −1.4 |
| AA469630_choline_kinase | 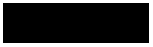 | 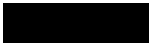 | −1.4 | 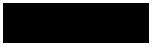 |
| AI1447967_ESTs | 1.1 | 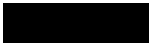 | 1.0557+ | 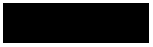 |
| AA754922_stearoyl-Coenzyme_A_desaturase_1 | −1.1 | −1.1 | −1.4 | 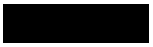 |
| AI614443_3′_phosphoadenosine_5′_phosphosulfate_synthase_2 | 1.2 | −1.2 | 1.5 | 1.2 |
| AI553232_ribosomal_protein_S6 | 1.2 | 1.5 | 1.0 | 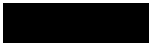 |
| AI876997_carboxyl_ester_lipase | 1.5 | 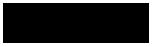 | −1.4 | −2.3 |
| AA561920_RaIBP1_associated_Eps_domain_containing_protein | 1.4 | 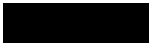 | −1.1 | 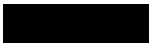 |
| AA544433_dihydroorotate_dehydrogenase | 1.2 | 1.5 | 1.0 | 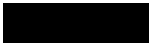 |
| AI037649_transition_protein_1 | −1.1 | 1.2 | −1.2 | 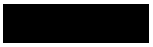 |
| AI550886_RIKEN_cDNA_1810010M01_gene | 1.1 | 1.4 | −1.2 | 1.4 |
| AA473562_ESTs,_Moderately_similar_to_alias_DLC1_[H.sapiens] | 1.3 | 1.5 | 1.2 | 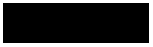 |
| AA623060_RIKEN_cDNA_2410124L17_gene | 1.5 | 1.5 | 1.1 | 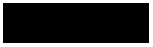 |
| AA869173_defensin_related_cryptdin_6 | −1.2 | 1.1 | −1.2 | 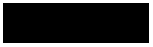 |
| AA588976_ESTs | 1.1 | 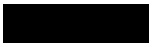 | −1.5 | 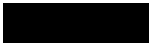 |
| AA517906_T-box_15 | 1.26469+ | 1.12314+ | −1.10579+ | 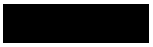 |
| AA153231_Public_domain_EST | 1.3 | 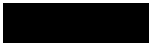 | 1.0 | 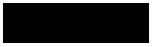 |
| AA517691_ESTs | 1.3 | 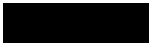 | −1.2 | 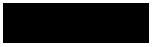 |
| AA073925_ESTs | 1.3 | 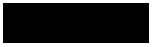 | −1.4 | 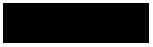 |
| AA473526_carnitine_acetyltranferase | −1.0 | 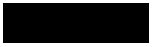 | −1.3 | 1.3 |
| AA789873_ATPase,_H+_transporting,_lysosomal_(vacuolar_pro- | −1.5 | 1.4 | 1.3 | 1.0 |
| ton_pump)_42kD | | | | |
| W08947_non-catalytic_region_of_tyrosine_kinase_adaptor_pro- | −1.8 | 1.1 | 1.3 | 1.0 |
| tein_2 | | | | |
| W36474_metallothionein_2 | −1.2 | −2.2 | 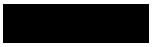 | 1.2 |
| AA867621_S-phase_kinase-associated_protein_2_(p45) | −1.8 | −1.2 | 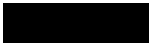 | 1.5 |
| AA638765_metallothionein_1 | 1.0 | 1.1 | 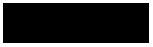 | 1.2 |
| AI608086_runt_related_transcription_factor_3 | −2.0 | 1.1 | 1.2 | 1.4 |
| AA821932_small_inducible_cytokine_A6 | 1.0 | −1.0 | 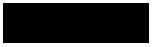 | −1.6 |
| AA684320_activin_receptor_interacting_protein_1 | −1.9 | 1.4 | 1.3 | 1.3 |
| AI325603_apolipoprotein_E | 1.2 | 1.3 | 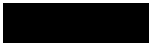 | −1.7 |
| AA822116_transthyretin | 1.2 | −1.1 | 1.5 | −1.5 |
| W99034_ESTs | −1.2 | 1.3 | 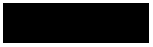 | 1.5 |
| AA066016_RIKEN_cDNA_1190002H23_gene | −1.6 | 1.2 | 1.2 | 1.5 |
| AA763276_small_muscle_protein,_X-linked | −1.7 | −1.4 | 1.1 | 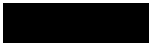 |
| AA607572_RIKEN_cDNA_1810017F10_gene | −1.3 | 1.1 | 1.4 | 1.1 |
| AA437863_RIKEN_cDNA_1200013I08_gene | −1.6 | 1.0 | 1.4 | 1.4 |
| AA497627_intersectin_(SH3_domain_protein_1A) | −1.6 | 1.0 | 1.1 | 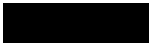 |
| AI047903_topoisomerase_(DNA)_III_beta | −1.7 | −1.0 | 1.4 | 1.1 |
| AA711868_sialoadhesin | −1.4 | 1.2 | 1.4 | 1.4 |
| AA013726_calhepsin_J | −1.0 | −1.0 | −1.2 | 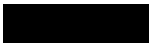 |
| AI428661_ESTs | −2.6 | −1.1 | 1.0 | −1.4 |
| AA880530_Public_domain_EST | −1.5 | −1.0 | 1.5 | 1.2 |
| AI324392_alpha_fetoprotein | −1.1 | −1.87 | −1.1 | −1.3 |
| W65070_ESTs | −1.2 | 1.4 | 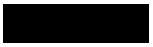 | 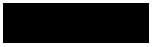 |
| AA024217_Public_domain_EST | −2.5 | 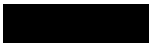 | 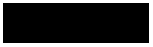 | 1.2 |
| AA474336_synaptonemal_complex_protein_3 | −2.8 | 1.2 | 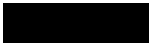 | 1.4 |
| AA624843_hemoglobin_Y,_beta-like_embryonic_chain | −1.4 | −1.6 | 1.1 | −1.2 |
| AA771229_ESTs,_Weakly_similar_to_zinc_finger_pro- | −2.4 | 1.2 | 1.3 | 1.2 |
| tein_95_[M.musculus] | | | | |
| AA272836_aquaporin_8 | −1.1 | 1.2 | −1.1 | 1.4 |
| AA546645_cell_division_cycle_2_homolog_(S._pombe)-like_2 | −2.1 | −1.1 | 1.3 | 1.4 |
| AA874101_tumor-associated_calcium_signal_transducer_2 | −2.6 | −1.1 | 1.5 | 1.3 |
| AA797886_ESTs,_Weakly_similar_to_ORF2_[M.musculus] | −1.8 | 1.4 | 1.2 | −1.0 |
| AA822098_apolipoprotein_A-1 | −1.9 | −1.5 | 1.1 | −2.4 |
| AA414078_DNA_segment,_Chr_7,_ERATO_Doi_486,_expressed | −1.7 | 1.2 | 1.4 | 1.2 |
| AA176045_forkhead_box_C2 | −2.5 | 1.1 | 1.4 | 1.4 |
| AA034678_rhotekin | −2.3 | 1.0 | 1.2 | −1.0 |
| AW209707_hemoglobin_Z,_beta-like_embryonic_chain | −1.5 | −1.5 | 1.1 | −1.8 |
| AA014727_tumor-suppressing_subchromosomal_transferable_frag- ment_3 | −2.1 | 1.1 | 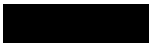 | −1.2 |
| AA795220_ADP-ribosylarginine_hydrolase | 1.0 | −1.1 | 1.2 | 1.1 |
| AA189266_ESTs | −3.2 | 1.4 | 1.5 | 1.5 |
| W56995_hemoglobin_X,_alpha-like_embry- | 1.1 | −1.6 | −1.3 | −1.2 |
| onic_chain_in_Hba_complex | | | | |

PLACENTA/UTERUS

| 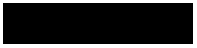 | int | col | br | lung |
|---|---|---|---|---|
| Description | PL-3B_BDEnor | PL-5B_BDEnor | PL-1B_BDEnor | PL-7B_BDEnor |
| 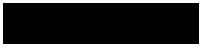 | 1.4283* | −1.60688+˜ | 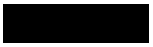 | 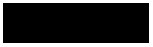 |
| 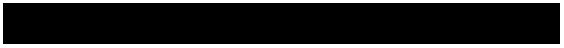 | 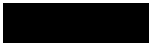 | −1.01549+˜ | 1.1 | 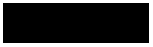 |
| AA270885_parvalbumin | 1.3 | 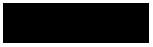 | −1.5 | 1.2 |
| AA469630_choline_kinase | 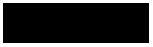 | 1.31548+˜ | 1.3 | 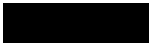 |
| AI1447967_ESTs | 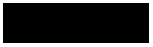 | −1.08065+˜ | 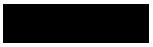 | 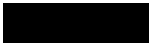 |
| AA754922_slearoyl-Coenzyme_A_desaturase_1 | 1.4 | 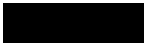 | −1.1 | −1.2 |
| AI614443_3′_phosphoadenosine_5′_phosphosulfate_synthase_2 | −3.7 | −1.4 | −1.3 | 1.4 |
| AI553232_ribosomal_protein_S6 | 1.2 | −1.4 | −1.3 | 1.4 |
| AI876997_carboxyl_ester_lipase | −8.9 | −1.9 | −1.3 | −1.4 |
| AA561920_RaIBP1_associated_Eps_domain_containing_protein | 1.5 | −1.1 | 1.2 | 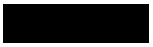 |
| AA544433_dihydroorotate_dehydrogenase | 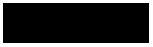 | 1.2437+˜ | 1.4 | 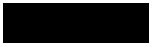 |
| AI037649_transition_protein_1 | 1.4 | −1.3 | −1.4 | 1.5 |
| AI550886_RIKEN_cDNA_1810010M01_gene | 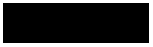 | −1.0 | −1.1 | 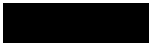 |
| AA473562_ESTs,_Moderately_similar_to_alias_DLC1_[H.sapiens] | 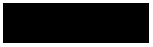 | −1.306+˜ | 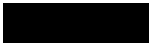 | 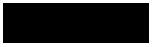 |
| AA623060_RIKEN_cDNA_2410124L17_gene | 1.36143* | −1.3 | 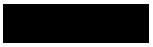 | 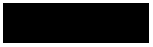 |
| AA869173_defensin_related_cryptdin_6 | 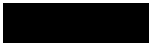 | 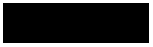 | −1.3 | 1.5 |
| AA588976_ESTs | 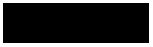 | 1.17361+˜ | 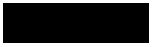 | 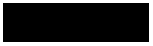 |
| AA517906_T-box_15 | 1.43344+ | −1.26463+˜ | 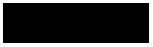 | 1.48842+ |
| AA153231_Public_domain_EST | 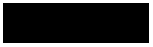 | −1.58578*˜ | 1.3 | 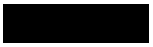 |
| AA517691_ESTs | 1.32145* | −1.2 | 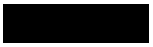 | 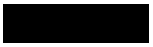 |
| AA073925_ESTs | 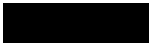 | −1.27077˜ | 1.1 | 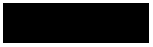 |
| AA473526_carnitine_acetyltranferase | 1.5 | −1.2 | 1.4 | 1.1 |
| AA789873_ATPase,_H+_transporting,_lysosomal_(vacuolar_pro- ton_pump)_42kD | 1.4 | 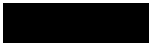 | −1.1 | −1.9 |
| W08947_non-catalytic_region_of_tyrosine_kinase_adaptor_pro- | 1.2 | 1.4 | −2.2 | −2.2 |
| tein_2 | | | | |
| W36474_metallothionein_2 | 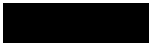 | 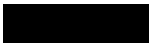 | 1.1 | −1.6 |
| AA867621_S-phase_kinase-associated_protein_2_(p45) | 1.2 | 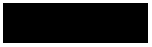 | −1.73102+ | −2.2 |
| AA638765_metallothionein_1 | 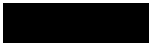 | 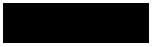 | 1.2 | −1.6 |
| AI608086_runt_related_transcription_factor_3 | 1.4 | 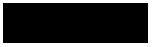 | −1.75763+ | −2.0 |
| AA821932_small_inducible_cytokine_A6 | 1.3 | 1.2 | −1.3 | −3.4 |
| AA684320_activin_receptor_interacting_protein_1 | 1.5 | 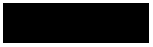 | 1.0 | −2.7 |
| AI325603_apolipoprotein_E | 1.5 | −1.0 | 1.5 | −1.9 |
| AA822116_transthyretin | −1.4 | 1.55627˜ | −1.3 | −7.7 |
| W99034_ESTs | 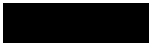 | 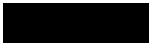 | 1.0 | −2.0 |
| AA066016_RIKEN_cDNA_1190002H23_gene | 1.5 | 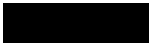 | −2.0 | −2.4 |
| AA763276_small_muscle_protein,_X-linked | 1.2 | 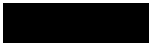 | −4.6 | −2.5 |
| AA607572_RIKEN_cDNA_1810017F10_gene | −1.0 | 1.50739˜ | 1.0 | −1.8 |
| AA437863_RIKEN_cDNA_1200013I08_gene | 1.3 | 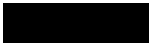 | −1.4 | −2.0 |
| AA497627_intersectin_(SH3_domain_protein_1A) | 1.5 | 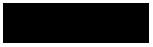 | −1.0 | −1.8 |
| AI047903_topoisomerase_(DNA)_III_beta | 1.1 | 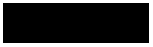 | −1.4 | −2.5 |
| AA711868_sialoadhesin | 1.4 | 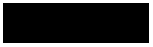 | −1.2 | −1.8 |
| AA013726_calhepsin_J | 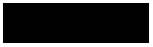 | −1.4 | −1.2 | 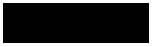 |
| AI428661_ESTs | 1.2 | 1.2 | −1.6 | −2.6 |
| AA880530_Public_domain_EST | 1.2 | 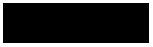 | 1.5 | −2.2 |
| AI324392_alpha_fetoprotein | −1.4 | −1.3 | −1.3 | −1.5 |
| W65070_ESTs | 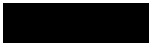 | 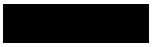 | 1.1 | −2.6 |
| AA024217_Public_domain_EST | 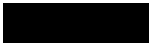 | 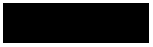 | −1.0 | −4.2 |
| AA474336_synaptonemal_complex_protein_3 | 1.5 | 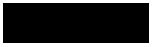 | 1.1 | −3.7 |
| AA624843_hemoglobin_Y,_beta-like_embryonic_chain | −1.1 | 1.3 | −1.7 | −1.6 |
| AA771229_ESTs,_Weakly_similar_to_zinc_finger_pro- tein_95_[M.musculus] | 1.2 | 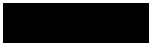 | −2.0 | −3.3 |
| AA272836_aquaporin_8 | 1.4 | −2.8 | 1.2 | 1.0 |
| AA546645_cell_division_cycle_2_homolog_(S._pombe)-like_2 | 1.1 | 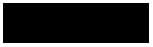 | −1.5 | −3.0 |
| AA874101_tumor-associated_calcium_signal_transducer_2 | 1.2 | 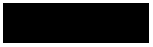 | −1.7 | −2.1 |
| AA797886_ESTs,_Weakly_similar_to_ORF2_[M.musculus] | 1.4 | 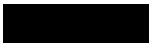 | −1.4 | −1.3 |
| AA822098_apolipoprotein_A-1 | 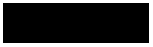 | 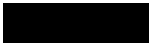 | −1.9 | −9.0 |
| AA414078_DNA_segment,_Chr_7,_ERATO_Doi_486,_expressed | 1.2 | 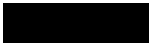 | 1.0 | −2.0 |
| AA176045_forkhead_box_C2 | 1.1 | 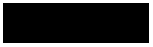 | −1.7 | −3.0 |
| AA034678_rhotekin | 1.2 | 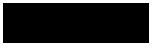 | −1.3 | −2.5 |
| AW209707_hemoglobin_Z,_beta-like_embryonic_chain | −1.4 | −1.80487˜ | 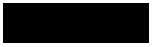 | −1.2 |
| AA014727_tumor-suppressing_subchromosomal_transferable_frag- ment_3 | 1.1 | 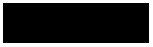 | −1.8 | −3.0 |
| AA795220_ADP-ribosylarginine_hydrolase | −1.1 | −1.29404˜ | 1.1 | 1.1 |
| AA189266_ESTs | 1.3 | 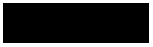 | −1.3 | −5.6 |
| W56995_hemoglobin_X,_alpha-like_embry- | −2.2 | 1.11232˜ | −2.1 | −2.0 |
| onic_chain_in_Hba_complex | | | | |

PLACENTA/UTERUS

| 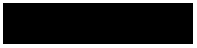 | blad | kidn | 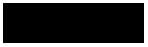 | mam |
|---|---|---|---|---|
| Description | PL-9B_BDEnor | PL-10B_BDEnor | PL-11B_BDEnor | PL-12B_BDEnor |
| 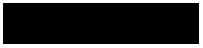 | 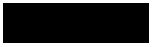 | 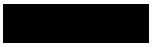 | 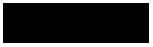 | 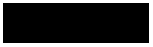 |
| 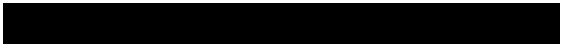 | 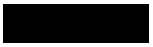 | 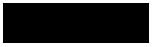 | 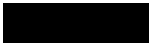 | 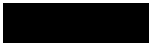 |
| AA270885_parvalbumin | 1.1 | 1.2 | 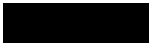 | −1.2 |
| AA469630_choline_kinase | 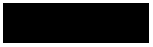 | 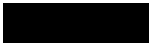 | 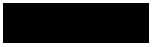 | 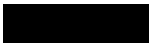 |
| AI1447967_ESTs | 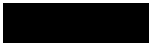 | 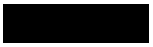 | 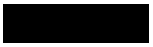 | 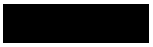 |
| AA754922_slearoyl-Coenzyme_A_desaturase_1 | −1.0 | −1.2 | 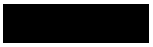 | −1.6 |
| AI614443_3′_phosphoadenosine_5′_phosphosulfate_synthase_2 | 1.5 | 1.3 | 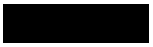 | 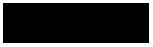 |
| AI553232_ribosomal_protein_S6 | 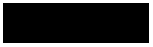 | 1.4 | 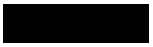 | 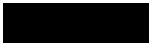 |
| AI876997_carboxyl_ester_lipase | −1.1 | 1.1 | 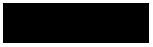 | 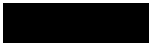 |
| AA561920_RaIBP1_associated_Eps_domain_containing_protein | 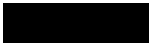 | 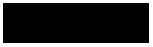 | 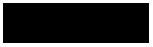 | 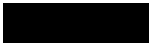 |
| AA544433_dihydroorotate_dehydrogenase | 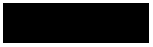 | 1.4 | 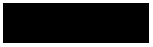 | 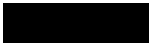 |
| AI037649_transition_protein_1 | 1.2 | 1.2 | 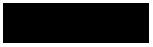 | 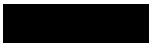 |
| AI550886_RIKEN_cDNA_1810010M01_gene | 1.2 | 1.2 | 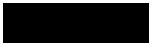 | 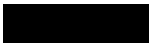 |
| AA473562_ESTs,_Moderately_similar_to_alias_DLC1_[H.sapiens] | 1.5 | 1.4 | 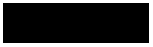 | 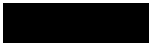 |
| AA623060_RIKEN_cDNA_2410124L17_gene | 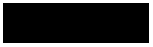 | 1.4 | 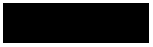 | 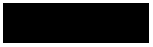 |
| AA869173_defensin_related_cryptdin_6 | 1.1 | 1.0 | 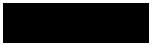 | 1.5 |
| AA588976_ESTs | 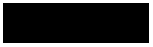 | 1.4 | 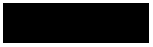 | 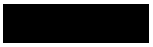 |
| AA517906_T-box_15 | 1.35698+ | 1.49743+ | 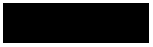 | 1.2 |
| AA153231_Public_domain_EST | 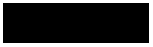 | 1.5 | 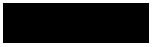 | 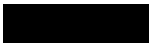 |
| AA517691_ESTs | 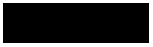 | 1.5 | 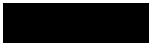 | 1.5 |
| AA073925_ESTs | 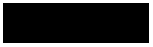 | 1.2 | 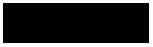 | 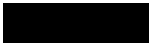 |
| AA473526_carnitine_acetyltranferase | 1.1 | 1.2 | 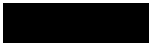 | −1.1 |
| AA789873_ATPase,_H+_transporting,_lysosomal_(vacuolar_pro- | −1.5 | −1.0 | −1.9 | −1.2 |
| ton_pump)_42kD | | | | |
| W08947_non-catalytic_region_of_tyrosine_kinase_adaptor_pro- | −1.8 | −1.3 | −1.9 | −1.2 |
| tein_2 | | | | |
| W36474_metallothionein_2 | −1.1 | 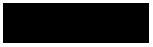 | −1.9 | 1.0 |
| AA867621_S-phase_kinase-associated_protein_2_(p45) | −1.8 | −1.4 | −1.9 | −1.3 |
| AA638765_metallothionein_1 | −1.3 | 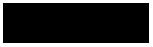 | −1.9 | −1.2 |
| AI608086_runt_related_transcription_factor_3 | −1.6 | −1.3 | −1.9 | −1.0 |
| AA821932_small_inducible_cytokine_A6 | −1.2 | 1.3 | −1.9 | −1.7 |
| AA684320_activin_receptor_interacting_protein_1 | −1.4 | −1.1 | −1.9 | −1.3 |
| AI325603_apolipoprotein_E | −1.3 | −1.5 | −2.0 | −1.2 |
| AA822116_transthyretin | −1.0 | 1.1 | −2.0 | −1.6 |
| W99034_ESTs | −1.4 | −1.3 | −2.0 | 1.0 |
| AA066016_RIKEN_cDNA_1190002H23_gene | −1.4 | −1.6 | −2.0 | −1.3 |
| AA763276_small_muscle_protein,_X-linked | −1.6 | −1.2 | −2.0 | −1.5 |
| AA607572_RIKEN_cDNA_1810017F10_gene | −1.9 | −1.3 | −2.0 | −1.6 |
| AA437863_RIKEN_cDNA_1200013I08_gene | −1.7 | −1.1 | −2.0 | −1.3 |
| AA497627_intersectin_(SH3_domain_protein_1A) | −1.5 | −1.2 | −2.1 | −1.2 |
| AI047903_topoisomerase_(DNA)_III_beta | −1.7 | −1.5 | −2.1 | −1.5 |
| AA711868_sialoadhesin | −1.4 | −1.2 | −2.1 | −1.4 |
| AA013726_calhepsin_J | 1.2 | 1.1 | −2.1 | 1.5 |
| AI428661_ESTs | −2.1 | −1.7 | −2.2 | −1.5 |
| AA880530_Public_domain_EST | −1.1 | −1.2 | −2.2 | −1.1 |
| AI324392_alpha_fetoprotein | −1.0 | −1.3 | −2.2 | −1.8 |
| W65070_ESTs | −1.4 | −1.4 | −2.3 | −1.1 |
| AA024217_Public_domain_EST | −1.9 | −1.2 | −2.3 | 1.1 |
| AA474336_synaptonemal_complex_protein_3 | −2.1 | −1.4 | −2.3 | −1.3 |
| AA624843_hemoglobin_Y,_beta-like_embryonic_chain | −1.3 | −1.0 | −2.3 | −1.5 |
| AA771229_ESTs,_Weakly_similar_to_zinc_finger_pro- | −2.2 | −1.4 | −2.4 | −1.4 |
| tein_95_[M.musculus] | | | | |
| AA272836_aquaporin_8 | 1.0 | 1.2 | −2.4 | 1.3 |
| AA546645_cell_division_cycle_2_homolog_(S._pombe)-like_2 | −2.3 | −1.5 | −2.4 | −1.4 |
| AA874101_tumor-associated_calcium_signal_transducer_2 | −1.5 | −1.5 | −2.4 | −1.2 |
| AA797886_ESTs,_Weakly_similar_to_ORF2_[M.musculus] | −1.7 | −1.5 | −2.4 | −1.2 |
| AA822098_apolipoprotein_A-1 | −1.2 | −2.1 | −2.5 | −1.6 |
| AA414078_DNA_segment,_Chr_7,_ERATO_Doi_486,_expressed | −1.4 | −1.1 | −2.5 | −1.2 |
| AA176045_forkhead_box_C2 | −2.3 | −1.6 | −2.5 | −1.1 |
| AA034678_rhotekin | −2.1 | −1.4 | −2.6 | −1.4 |
| AW209707_hemoglobin_Z,_beta-like_embryonic_chain | −1.4 | 1.1 | −2.7 | −1.9 |
| AA014727_tumor-suppressing_subchromosomal_transferable_frag- | −2.1 | −1.8 | −2.9 | −1.0 |
| ment_3 | | | | |
| AA795220_ADP-ribosylarginine_hydrolase | 1.1 | 1.0 | −3.3 | −1.0 |
| AA189266_ESTs | −3.0 | −1.8 | −3.9 | −1.4 |
| W56995_hemoglobin_X,_alpha-like_embry- | 1.0 | −1.2 | −4.0 | −1.6 |
| onic_chain_in_Hba_complex | | | | |

KIDNEY

| 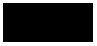 | spl | panc | liv | stom |
|---|---|---|---|---|
| Description | PL-4B_BDEnor | PL-2B_BDEnor | PL-6B_BDEnor | PL-8B_BDEnor |
| 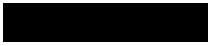 | −1.67339+ | −1.41617+ | −1.23697+ | 1.12201+ |
| 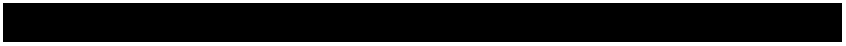 | −1.1 | −2.0 | −1.8 | 1.0 |
| 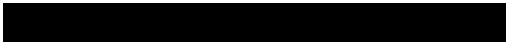 | −1.2 | −1.1 | 1.2 | −1.1 |
| AA106125_cytochrome_P450,_2a4 | 1.3 | −1.3 | 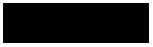 | 1.3 |
| AI155437_Mus_musculus_20alpha-HSD_mRNA_for_20-alpha- | 1.3 | −1.5 | −1.2 | 1.1 |
| hydroxysteroid_dehydrogenase,_complete_cds | | | | |
| AI614454_reduced_in_osleosclerosis_transporter | 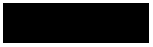 | −1.1 | −1.8 | 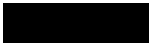 |
| AA821997_myoglobin | −1.0 | −2.5 | 1.0 | 1.1 |
| W16059_glutathione_S-transferase_omega_1 | 1.2 | −1.1 | 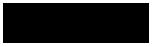 | −1.4 |
| AA674177_cytochrome_P450,_2a4 | 1.2 | −1.6 | 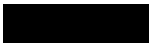 | 1.1 |
| AA087441_RIKEN_cDNA_0610011L04_gene | 1.2 | 1.1 | 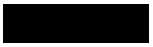 | 1.3 |
| AA146478_M.musculus_mRNA_(3C10)_for_IgA_V-D-J- heavy_chain | −1.3 | 1.0 | −1.6 | 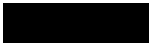 |
| AA096815_Public_domain_EST | 1.2 | −1.3 | 1.2 | 1.5 |
| AA145454_insulin-like_growth_factor_binding_protein_4 | 1.0 | −1.5 | 1.3 | −1.2 |
| AI549666_ESTs | 1.35481+ | 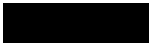 | 1.04863+ | 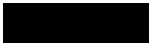 |
| AA108417_insulin-like_growth_factor_binding_protein_4 | −1.0 | −1.5 | 1.2 | −1.2 |
| AA450534_glutamine_synthetase_pseudogene_1 | 1.1 | −1.1 | 1.2 | 1.4 |
| AI552658_ESTS | −1.3 | 1.1 | −1.2 | 1.2 |
| AI390138_RIKEN_cDNA_3930401B19_gene | −1.4 | −10.7 | −1.1 | −2.0 |
| AA681098_ESTs | −1.13993+ | −1.31577+ | 1.39877+ | 1.02396+ |
| AI385457_retinol_binding_protein_2,_cellular | −2.0 | −2.1 | −1.6 | −1.9 |
| AI894032_trefoil_factor_2_(spasmolytic_protein_1) | −1.1 | 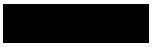 | −1.2 | −2.0 |
| AI597097_Public_domain_EST | 1.3 | 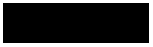 | −1.1 | −1.0 |
| AI036489_cyclin_G | −1.2 | 1.0 | 1.3 | −1.9 |
| AI385510_cadherin_13 | −1.2 | −1.6 | −1.0 | 1.2 |
| AI536309_RIKEN_cDNA_2210410L06_gene | −1.8 | −1.8 | −1.4 | −2.1 |
| AA822098_apolipoprotein_A-I | −1.9 | −1.5 | 1.1 | −2.4 |
| AA684191_CDC-like_kinase | 1.5 | −2.1 | −1.3 | −1.1 |
| AA822105_major_urinary_protein_1 | −1.1 | −2.4 | −28.7649* | −7.8 |
| AA434680_ESTs | −1.1 | −1.1 | −1.0 | −1.0 |
| AA108495_deoxyribonuclease_I | 1.1 | −1.8 | −1.1 | 1.2 |
| AA184421_ESTs | −1.1 | 1.0 | −1.0 | 1.0 |
| AA060880_RIKEN_cDNA_1700023M09_gene | −1.1 | −1.8 | −1.1 | 1.4 |

KIDNEY

| 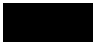 | int | col | br | lung |
|---|---|---|---|---|
| Description | PL-3B_BDEnor | PL-5B_BDEnor | PL-1B_BDEnor | PL-7B_BDEnor |
| 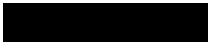 | −1.35665+ | −1.61968+˜ | 1.32548+ | −1.24845+ |
| 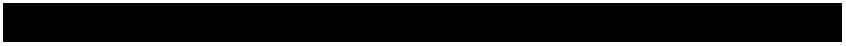 | −1.8 | −2.3 | −1.5 | −2.4 |
| 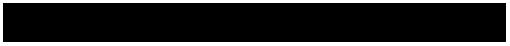 | −1.1 | 1.4 | −1.4 | 1.1 |
| AA106125_cytochrome_P450,_2a4 | 1.4 | 1.0 | −2.4 | 1.3 |
| AI155437_Mus_musculus_20alpha-HSD_mRNA_for_20-alpha- | 1.3 | 1.15702+˜ | −1.2 | 1.1 |
| hydroxysteroid_dehydrogenase,_complete_cds | | | | |
| AI614454_reduced_in_osleosclerosis_transporter | 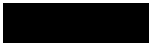 | 1.11125˜ | 1.3 | 1.2 |
| AA821997_myoglobin | 1.0 | −1.2 | −1.8 | −1.7 |
| W16059_glutathione_S-transferase_omega_1 | −1.1 | −1.2 | 1.2 | 1.2 |
| AA674177_cytochrome_P450,_2a4 | 1.3 | 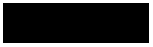 | −2.1 | 1.2 |
| AA087441_RIKEN_cDNA_0610011L04_gene | −1.1 | 1.1497˜ | −1.0 | 1.2 |
| AA146478_M.musculus_mRNA_(3C10)_for_IgA_V-D-J- | −2.0 | −2.4 | −1.1 | −1.4 |
| heavy_chain | | | | |
| AA096815_Public_domain_EST | 1.2 | 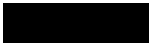 | −1.9 | −1.2 |
| AA145454_insulin-like_growth_factor_binding_protein_4 | −1.1 | 1.0 | −1.1 | 1.0 |
| AI549666_ESTs | 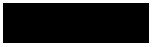 | 1.52793+˜ | 1.07014+ | 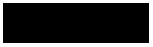 |
| AA108417_insulin-like_growth_factor_binding_protein_4 | −1.1 | −1.3 | 1.0 | −1.2 |
| AA450534_glutamine_synthetase_pseudogene_1 | 1.2 | −1.3 | 1.4 | 1.4 |
| AI552658_ESTS | −1.1 | 1.06684+˜ | 1.2 | −1.1 |
| AI390138_RIKEN_cDNA_3930401B19_gene | −1.2 | −2.1 | −1.3 | −1.2 |
| AA681098_ESTs | −1.06627+ | 1.49883+˜ | −1.69915+ | 1.35989+ |
| AI385457_retinol_binding_protein_2,_cellular | 1.5 | 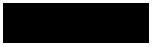 | −1.3 | −1.9 |
| AI894032_trefoil_factor_2_(spasmolytic_protein_1) | −82.1 | −1.89777˜ | −1.4 | 1.0 |
| AI597097_Public_domain_EST | −1.6 | 1.2 | 1.2 | −1.0 |
| AI036489_cyclin_G | −1.8 | −2.26973˜ | 1.2 | −1.4 |
| AI385510_cadherin_13 | −1.5 | −1.0 | −1.1 | 1.1 |
| AI536309_RIKEN_cDNA_2210410L06_gene | −1.9 | 1.1 | −1.5 | −2.0 |
| AA822098_apolipoprotein_A-I | 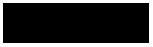 | 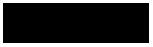 | −1.9 | −9.0 |
| AA684191_CDC-like_kinase | −2.0 | −1.4 | −1.3 | 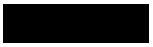 |
| AA822105_major_urinary_protein_1 | −1.1 | 1.43224˜ | −1.3 | −71.1 |
| AA434680_ESTs | −1.7 | 1.2 | −1.0 | −1.0 |
| AA108495_deoxyribonuclease_I | 1.4 | 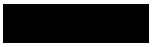 | −2.3 | −1.2 |
| AA184421_ESTs | −1.4 | 1.1 | 1.4 | 1.2 |
| AA060880_RIKEN_cDNA_1700023M09_gene | −1.5 | 1.0 | −1.3 | 1.1 |

KIDNEY

| 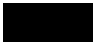 | blad | 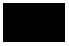 | pl/ut | mam |
|---|---|---|---|---|
| Description | PL-9B_BDEnor | PL-10B_BDEnor | PL-11B_BDEnor | PL-12B_BDEnor |
| 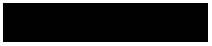 | 1.3 | 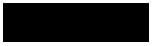 | −1.1 | 1.4 |
| 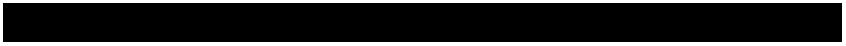 | −1.4 | 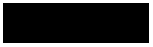 | −1.4 | −1.4 |
| 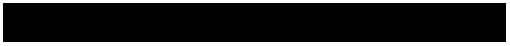 | 1.1 | 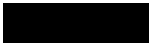 | −1.11695* | −1.1 |
| AA106125_cytochrome_P450,_2a4 | 1.1 | 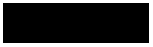 | 1.1 | 1.1 |
| AI155437_Mus_musculus_20alpha-HSD_mRNA_for_20-alpha- hydroxysteroid_dehydrogenase,_complete_cds | −1.0 | 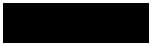 | −1.2 | 1.1 |
| AI614454_reduced_in_osleosclerosis_transporter | 1.2 | 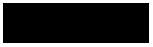 | 1.5 | 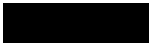 |
| AA821997_myoglobin | −4.3 | 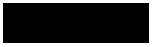 | −1.1 | −1.7 |
| W16059_glutathione_S-transferase_omega_1 | 1.4 | 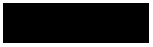 | −1.0 | 1.0 |
| AA674177_cytochrome_P450,_2a4 | −1.0 | 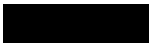 | −1.1 | −1.2 |
| AA087441_RIKEN_cDNA_0610011L04_gene | 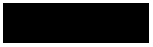 | 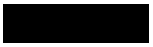 | 1.2 | −1.1 |
| AA146478_M.musculus_mRNA_(3C10)_for_IgA_V-D-J- heavy_chain | 1.5 | 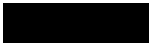 | 1.1 | 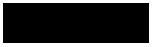 |
| AA096815_Public_domain_EST | 1.3 | 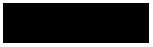 | 1.0 | 1.4 |
| AA145454_insulin-like_growth_factor_binding_protein_4 | 1.4 | 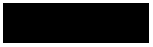 | −1.0 | −1.2 |
| AI549666_ESTs | 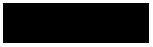 | 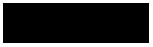 | 1.5 | 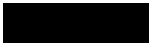 |
| AA108417_insulin-like_growth_factor_binding_protein_4 | 1.2 | 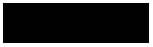 | −1.1 | −1.3 |
| AA450534_glutamine_synthetase_pseudogene_1 | 1.3 | 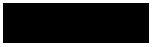 | 1.4 | 1.1 |
| AI552658_ESTS | −1.0 | 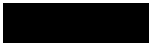 | −1.3 | −1.0 |
| AI390138_RIKEN_cDNA_3930401B19_gene | 1.5 | −1.9 | −1.3 | −1.1 |
| AA681098_ESTs | 1.13503+ | −1.9 | 1.3409+ | −1.19374+ |
| AI385457_retinol_binding_protein_2,_cellular | −1.6 | −1.9 | −1.5 | −1.7 |
| AI894032_trefoil_factor_2_(spasmolytic_protein_1) | −1.4 | −1.9 | −1.0 | 1.4 |
| AI597097_Public_domain_EST | 1.2 | −1.9 | 1.0 | −1.3 |
| AI036489_cyclin_G | −1.4 | −2.0 | −1.0 | −1.1 |
| AI385510_cadherin_13 | −1.6 | −2.1 | −1.2 | −1.0 |
| AI536309_RIKEN_cDNA_2210410L06_gene | −1.6 | −2.1 | −1.5 | −2.1 |
| AA822098_apolipoprotein_A-I | −1.2 | −2.1 | −2.5 | −1.6 |
| AA684191_CDC-like_kinase | 1.4 | −2.1 | 1.4 | 1.2 |
| AA822105_major_urinary_protein_1 | −1.6 | −2.2 | −1.1 | −1.2 |
| AA434680_ESTs | −1.1 | −2.2 | −1.0 | −1.2 |
| AA108495_deoxyribonuclease_I | 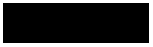 | −2.4 | 1.3 | 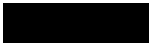 |
| AA184421_ESTs | −1.3 | −2.4 | −1.1 | −1.1 |
| AA060880_RIKEN_cDNA_1700023M09_gene | −1.7 | −2.7 | −1.1 | 1.1 |

BLADDER

| 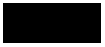 | spl | panc | liv | stom |
|---|---|---|---|---|
| Description | PL-4B_BDEnor | PL-2B_BDEnor | PL-6B_BDEnor | PL-8B_BDEnor |
| 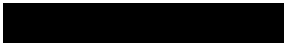 | 1.2 | 1.1 | −1.1 | −1.4 |
| 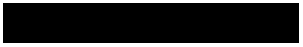 | 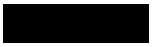 | 1.5 | −1.2 | −1.4 |
| 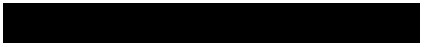 | −1.2 | −1.7 | −1.0 | −1.2 |
| 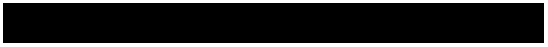 | −1.1 | −1.2 | −1.1 | 1.1 |
| 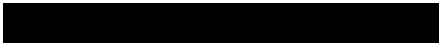 | 1.3 | −1.4 | −1.2 | 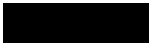 |
| 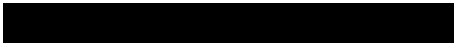 | 1.1 | 1.1 | −1.1 | −1.1 |
| 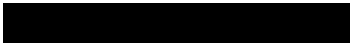 | −1.1 | −1.3 | 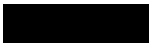 | −1.2 |
| W11432_Leman_coiled-coil_protein | −1.1 | −1.3 | 1.2 | −1.1 |
| AA619838_ESTs | 1.08217+ | −1.76658+ | 1.14152+ | −1.0 |
| AA756749_submaxillary_gland_androgen_regulated_protein_1 | −1.1 | −1.6 | 1.0 | −1.3 |
| AI552219_ESTs | 1.2087+ | 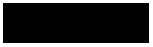 | −1.18563+ | 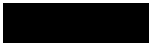 |
| AI549624_RIKEN_cDNA_0610041E09_gene | 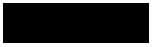 | 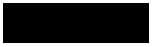 | −1.3 | 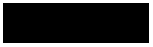 |
| AA623175_ESTs | 1.5 | 1.5 | −1.0 | 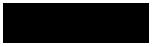 |
| AA469630_choline_kinase | 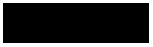 | 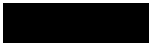 | −1.4 | 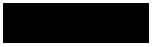 |
| AI553220_ESTs | 1.47814* | −1.0 | 1.3 | 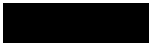 |
| AA469724_WW_domain_binding_protein_5 | 1.3 | 1.4 | −1.1 | 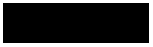 |
| AA153231_Public_domain_EST | 1.3 | 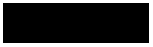 | 1.0 | 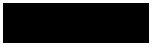 |
| AA155097_ESTs | 1.4 | 1.1 | 1.1 | 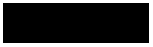 |
| AA445667_RIKEN_cDNA_4632401C08_gene | 1.3 | −1.2 | −2.0 | 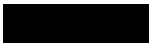 |
| AA6230_RaIBP1_associated_Eps_domain_containing_protein | 1.4 | 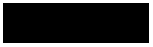 | −1.1 | 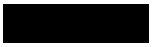 |
| W82228_annexin_A3 | 1.5 | −1.4 | 1.0 | −1.7 |
| AA073925_ESTs | 1.3 | 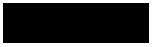 | −1.4 | 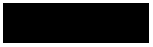 |
| AA607572_RIKEN_cDNA_1810017F10_gene | −1.3 | 1.1 | 1.4 | 1.1 |
| AA199011_histocompatibility_2,_class_II_antigen_A,_alpha | −1.1 | −1.3 | 1.1 | 1.0 |
| AA880220_jagged_1 | −1.3 | −1.1 | −1.2 | −1.4 |
| AA024217_Public_domain_EST | −2.5 | 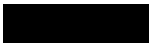 | 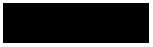 | 1.2 |
| AA681602_enolase_3,_beta_muscle | 1.1 | −1.7 | −1.0 | −1.4 |
| AA759679_la-associated_invariant_chain | −1.0 | −1.8 | −1.6 | −1.9 |
| AA414790_leukotriene_A4_hydrolase | −2.1 | −2.2 | −1.6 | −2.8 |
| AA982254_RIKEN_cDNA_2210010C04_gene | 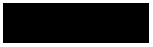 | 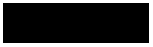 | −1.3 | −2.0 |
| AI390242_RIKEN_cDNA_2310005E10_gene | 1.1 | −1.1 | −1.2 | −1.3 |
| AA839138_CD8_antigen,_alpha_chain | −1.3 | −1.08773+ | −1.3 | −1.8 |
| AA474336_synaptonemal_complex_protein_3 | −2.8 | 1.2 | 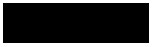 | 1.4 |
| AA671135_myosin_heavy_chain,_cardiac_muscle,_adult | −1.1 | −1.7 | 1.3 | 1.2 |
| AA770924_troponin_T3,_skeletal,_fast | 1.4 | −1.5 | 1.1 | 1.2 |
| AA034678_rhotekin | −2.3 | 1.0 | 1.2 | −1.0 |
| AI428661_ESTs | −2.6 | −1.1 | 1.0 | −1.4 |
| AA014727_tumor-suppressing_subchromosomal_transferable_frag- ment_3 | −2.1 | 1.1 | 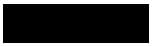 | −1.2 |
| AA667301_troponin_I,_cardiac | 1.4 | −1.6 | −1.1 | −2.1 |
| AA675111_RIKEN_cDNA_1810007A24_gene | 1.4 | 1.3 | −1.0 | −2.1 |
| AA881388_ESTs,_Weakly_similar_to_zinc_finger_pro- | −2.4 | 1.2 | 1.3 | 1.2 |
| tein_95_[M.musculus] | | | | |
| AA881388_ESTs | −1.1 | 1.0 | −1.1 | −1.2 |
| W10293_androgen_regulated_vas_deferens_protein | 1.2 | 1.4 | −1.1 | 1.4 |
| AA176045_forkhead_box_C2 | −2.5 | 1.1 | 1.4 | 1.4 |
| AA546645_cell_division_cycle_2_homolog_(S._pombe)-like_2 | −2.1 | −1.1 | 1.3 | 1.4 |
| AA221794_regulator_of_G-protein_signaling_2 | 1.0 | −1.7 | −1.4 | −1.3 |
| AI451862_RIKEN_cDNA_0910001A18_gene | 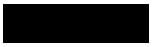 | 1.3 | −1.1 | −2.5 |
| AI892132_cytochrome_c_oxidase,_subunit_VI_a,_polypeptide_2 | 1.1 | −1.3 | 1.1 | −1.0 |
| AA065510_defensin_beta_1 | 1.4 | −1.4 | 1.1 | −1.0 |
| AI386046_trypsin_4 | 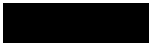 | 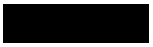 | 1.1 | −1.8 |
| W15001_CD52_antigen | 1.1 | −1.7 | 1.2 | 1.1 |
| AA238062_CD52_antigen | 1.1 | −1.6 | −1.3 | 1.0 |
| AA237793_rat_regenerating_islet-derived,_mouse_homolog_1 | 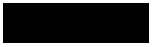 | 1.4 | −1.3 | −3.9 |
| AA717025_Mus_musculus_10_day_old_male_pan- creas_cDNA,_RIKEN_fulllength_enriched_library,_clone: 1810009A17,_full_insert_sequence | 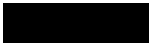 | −1.1 | −1.2 | −2.7 |
| AA771566_troponin_C,_fast_skeletal | 1.1 | −1.6 | 1.4 | −1.1 |
| AA189266_ESTs | −3.2 | 1.4 | 1.5 | 1.5 |
| AA097074_aquaporin_2 | −1.1 | −1.0 | 1.5 | 1.4 |
| AA712003_resistin_like_alpha | 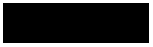 | −1.4 | −1.1 | 1.3 |
| AA675084_elastase_2 | 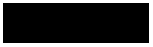 | 1.4 | −1.2 | −2.7 |
| AA790398_ribonuclease_1,_pancreatic | 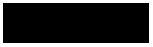 | 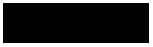 | −1.2 | −5.8 |
| AA821997_myoglobin | −1.0 | −2.5 | 1.0 | 1.1 |
| AA821884_amylase_2,_pancreatic | 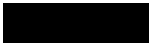 | 1.1 | −1.2 | −2.8 |
| AA242201_serine_protease_inhibitor,_Kazal_type_3 | 1.5 | 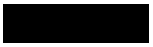 | −1.4 | 1.1 |

BLADDER

| 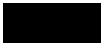 | int | col | br | lung |
|---|---|---|---|---|
| Description | PL-3B_BDEnor | PL-5B_BDEnor | PL-1B_BDEnor | PL-7B_BDEnor |
| 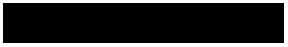 | −1.9 | −1.2 | −1.5 | 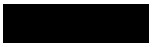 |
| 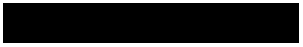 | −2.7 | −2.3 | −1.2 | 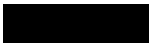 |
| 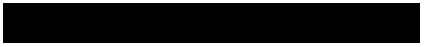 | −1.2 | 1.5 | −2.0 | 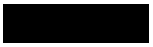 |
| 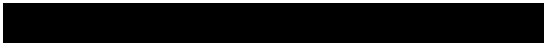 | 1.1 | 1.1 | −1.4 | 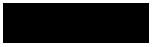 |
| 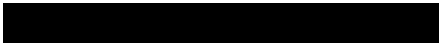 | −1.1 | −1.4 | −1.4 | 1.5 |
| 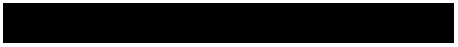 | 1.1 | −1.03021+˜ | −1.0 | 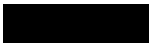 |
| 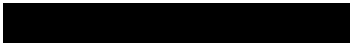 | 1.2 | 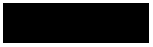 | −2.3 | 1.2 |
| W11432_Leman_coiled-coil_protein | −1.1 | −1.06612˜ | −1.1 | 1.3 |
| AA619838_ESTs | −1.10855+ | −1.28428+˜ | 1.00186+ | −1.11217+ |
| AA756749_submaxillary_gland_androgen_regulated_protein_1 | −1.2 | 1.5 | −2.0 | 1.3 |
| AI552219_ESTs | 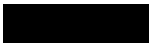 | −1.26743+˜ | 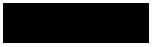 | 1.53405+ |
| AI549624_RIKEN_cDNA_0610041E09_gene | 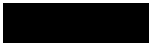 | −1.01549+˜ | 1.1 | 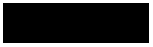 |
| AA623175_ESTs | 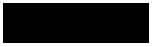 | −1.30902+˜ | 1.4 | 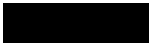 |
| AA469630_choline_kinase | 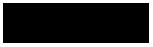 | 1.31548+˜ | 1.3 | 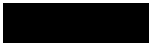 |
| AI553220_ESTs | 1.16813* | −1.30242˜ | −1.1 | 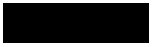 |
| AA469724_WW_domain_binding_protein_5 | 1.5 | 1.0 | 1.1 | 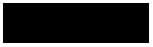 |
| AA153231_Public_domain_EST | 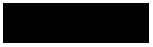 | −1.58578*˜ | 1.3 | 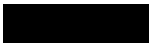 |
| AA155097_ESTs | 1.2 | −1.2 | 1.4 | 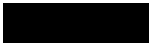 |
| AA445667_RIKEN_cDNA_4632401C08_gene | −1.0 | 1.1 | 1.3 | −1.2 |
| AA6230_RaIBP1_associated_Eps_domain_containing_protein | 1.5 | −1.1 | 1.2 | 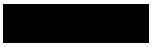 |
| W82228_annexin_A3 | −1.7 | −1.86744+˜ | 1.1 | 1.4 |
| AA073925_ESTs | 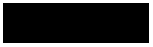 | −1.27077˜ | 1.1 | 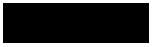 |
| AA607572_RIKEN_cDNA_1810017F10_gene | −1.0 | 1.50739˜ | 1.0 | −1.8 |
| AA199011_histocompatibility_2,_class_II_antigen_A,_alpha | 1.3 | −4.5 | 1.2 | 1.1 |
| AA880220_jagged_1 | 1.1 | −1.6 | 1.3 | −1.2 |
| AA024217_Public_domain_EST | 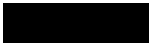 | 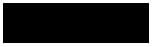 | −1.0 | −4.2 |
| AA681602_enolase_3,_beta_muscle | 1.3 | −1.5 | −2.5 | −1.3 |
| AA759679_la-associated_invariant_chain | 1.5 | −3.7 | −2.8 | −1.0 |
| AA414790_leukotriene_A4_hydrolase | −1.3 | −1.6 | −1.2 | −1.7 |
| AA982254_RIKEN_cDNA_2210010C04_gene | −9.5 | −1.3 | 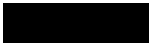 | −1.5 |
| AI390242_RIKEN_cDNA_2310005E10_gene | −1.3 | −1.56397˜ | −1.1 | −1.0 |
| AA839138_CD8_antigen,_alpha_chain | 1.2 | −2.63867+˜ | −4.0 | 1.0 |
| AA474336_synaptonemal_complex_protein_3 | 1.5 | 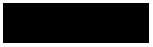 | 1.1 | −3.7 |
| AA671135_myosin_heavy_chain,_cardiac_muscle,_adult | 1.1 | 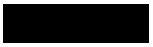 | −1.9 | −1.3 |
| AA770924_troponin_T3,_skeletal,_fast | 1.0 | −1.7 | −1.8 | −1.1 |
| AA034678_rhotekin | 1.2 | 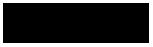 | −1.3 | −2.5 |
| AI428661_ESTs | 1.2 | 1.2 | −1.6 | −2.6 |
| AA014727_tumor-suppressing_subchromosomal_transferable_frag- ment_3 | 1.1 | 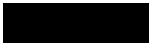 | −1.8 | −3.0 |
| AA667301_troponin_I,_cardiac | 1.3 | 1.41297˜ | −2.0 | −2.0 |
| AA675111_RIKEN_cDNA_1810007A24_gene | −14.0 | −1.5 | 1.0 | −1.4 |
| AA881388_ESTs,_Weakly_similar_to_zinc_finger_pro- tein_95_[M.musculus] | 1.2 | 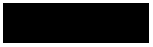 | −2.0 | −3.3 |
| AA881388_ESTs | 1.1 | −1.2 | 1.2 | −1.28277* |
| W10293_androgen_regulated_vas_deferens_protein | 1.2 | −1.3 | −1.3 | 1.5 |
| AA176045_forkhead_box_C2 | 1.1 | 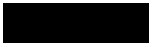 | −1.7 | −3.0 |
| AA546645_cell_division_cycle_2_homolog_(S._pombe)-like_2 | 1.1 | 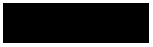 | −1.5 | −3.0 |
| AA221794_regulator_of_G-protein_signaling_2 | 1.2 | −1.29501˜ | 1.0 | −1.0 |
| AI451862_RIKEN_cDNA_0910001A18_gene | −14.7 | −2.6 | 1.2 | −1.3 |
| AI892132_cytochrome_c_oxidase,_subunit_VI_a,_polypeptide_2 | 1.1 | −1.57226˜ | −1.2 | −1.7 |
| AA065510_defensin_beta_1 | 1.2 | 1.33028˜ | −1.8 | −1.2 |
| AI386046_trypsin_4 | −1.59 | −2.6 | 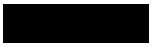 | −1.5 |
| W15001_CD52_antigen | 1.1 | −2.3 | −1.2 | −1.2 |
| AA238062_CD52_antigen | −1.1 | −1.9 | −1.6 | −1.3 |
| AA237793_rat_regenerating_islet-derived,_mouse_homolog_1 | −11.8 | −2.2 | 1.0 | −1.4 |
| AA717025_Mus_musculus_10_day_old_male_pan- creas_cDNA,_RIKEN_fulllength_enriched_library,_clone: 1810009A17,_full_insert_sequence | −25.5 | −5.2 | 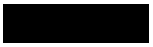 | −1.6 |
| AA771566_troponin_C,_fast_skeletal | 1.0 | 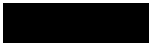 | −2.0 | −1.2 |
| AA189266_ESTs | 1.3 | 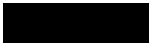 | −1.3 | −5.6 |
| AA097074_aquaporin_2 | 1.5 | 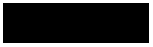 | −1.2 | −1.1 |
| AA712003_resistin_like_alpha | 1.1 | −1.65763˜ | 1.0 | −1.3 |
| AA675084_elastase_2 | −21.7 | −2.2 | 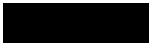 | −1.9 |
| AA790398_ribonuclease_1,_pancreatic | −16.6 | −3.3 | 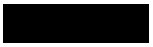 | −1.9 |
| AA821997_myoglobin | 1.0 | −1.2 | −1.8 | −1.7 |
| AA821884_amylase_2,_pancreatic | −32.0 | −4.0 | 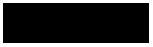 | −1.2 |
| AA242201_serine_protease_inhibitor,_Kazal_type_3 | 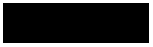 | −1.0 | −1.8 | 1.2 |

BLADDER

| 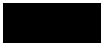 | 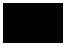 | kidn | pl/ut | mam |
|---|---|---|---|---|
| Description | PL-9B_BDEnor | PL-10B_BDEnor | PL-11B_BDEnor | PL-12B_BDEnor |
| 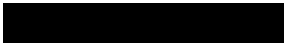 | 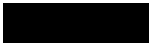 | 1.1 | −1.1 | −1.4 |
| 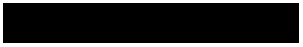 | 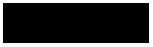 | 1.1 | 1.0 | 1.3 |
| 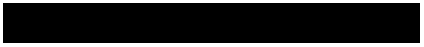 | 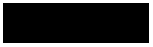 | −1.6 | −1.2 | −1.4 |
| 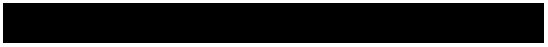 | 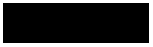 | −1.4 | 1.1 | 1.1 |
| 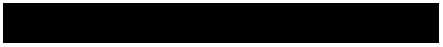 | 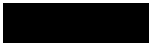 | 1.3 | 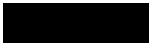 | 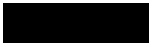 |
| 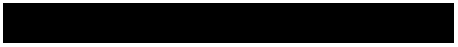 | 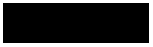 | −1.0 | 1.1 | 1.2 |
| 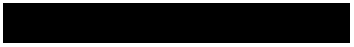 | 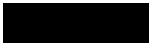 | −1.2 | −1.2 | −1.0 |
| W11432_Leman_coiled-coil_protein | 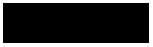 | −1.1 | 1.2 | 1.2 |
| AA619838_ESTs | 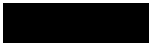 | 1.3 | −1.11428+ | −1.3 |
| AA756749_submaxillary_gland_androgen_regulated_protein_1 | 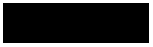 | −1.2 | −1.2 | −1.8 |
| AI552219_ESTs | 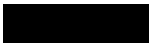 | 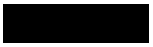 | 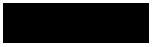 | 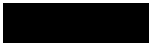 |
| AI549624_RIKEN_cDNA_0610041E09_gene | 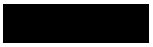 | 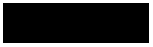 | 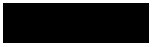 | 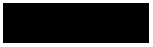 |
| AA623175_ESTs | 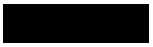 | 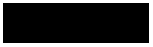 | 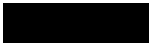 | 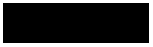 |
| AA469630_choline_kinase | 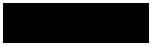 | 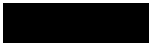 | 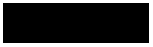 | 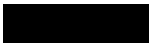 |
| AI553220_ESTs | 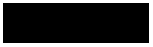 | 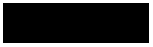 | 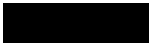 | 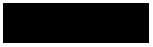 |
| AA469724_WW_domain_binding_protein_5 | 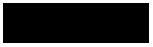 | 1.3 | 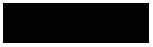 | 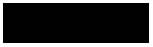 |
| AA153231_Public_domain_EST | 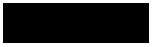 | 1.5 | 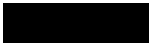 | 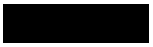 |
| AA155097_ESTs | 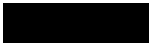 | 1.2 | 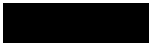 | 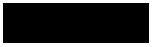 |
| AA445667_RIKEN_cDNA_4632401C08_gene | 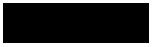 | 1.4 | 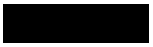 | 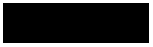 |
| AA6230_RaIBP1_associated_Eps_domain_containing_protein | 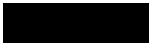 | 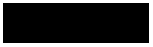 | 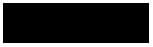 | 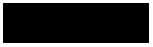 |
| W82228_annexin_A3 | 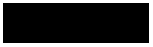 | 1.0 | 1.3 | 1.1 |
| AA073925_ESTs | 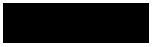 | 1.2 | 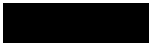 | 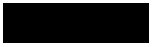 |
| AA607572_RIKEN_cDNA_1810017F10_gene | −1.9 | −1.3 | −2.0 | −1.6 |
| AA199011_histocompatibility_2,_class_II_antigen_A,_alpha | −1.9 | 1.3 | −1.0 | −1.2 |
| AA880220_jagged_1 | −1.9 | −1.0 | −1.5 | −1.3 |
| AA024217_Public_domain_EST | −1.9 | −1.2 | −2.3 | 1.1 |
| AA681602_enolase_3,_beta_muscle | −1.9 | 1.1 | 1.2 | −1.4 |
| AA759679_la-associated_invariant_chain | −1.9 | −1.0 | −1.3 | −1.4 |
| AA414790_leukotriene_A4_hydrolase | −2.0 | −1.7 | −1.6 | −1.8 |
| AA982254_RIKEN_cDNA_2210010C04_gene | −2.0 | −1.4 | −1.1 | −1.1 |
| AI390242_RIKEN_cDNA_2310005E10_gene | −2.0 | −1.1 | −1.0 | −1.1 |
| AA839138_CD8_antigen,_alpha_chain | −2.0 | −1.1 | 1.0 | −1.1 |
| AA474336_synaptonemal_complex_protein_3 | −2.1 | −1.4 | −2.3 | −1.3 |
| AA671135_myosin_heavy_chain,_cardiac_muscle,_adult | −2.1 | 1.4 | −1.2 | −1.3 |
| AA770924_troponin_T3,_skeletal,_fast | −2.1 | 1.0 | 1.2 | −1.2 |
| AA034678_rhotekin | −2.1 | −1.4 | −2.6 | −1.4 |
| AI428661_ESTs | −2.1 | −1.7 | −2.2 | −1.5 |
| AA014727_tumor-suppressing_subchromosomal_transferable_frag- | −2.1 | −1.8 | −2.9 | −1.0 |
| ment_3 | | | | |
| AA667301_troponin_I,_cardiac | −2.2 | 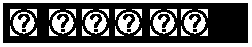 | −1.0 | 1.3 |
| AA675111_RIKEN_cDNA_1810007A24_gene | −2.2 | −1.2 | −1.2 | −2.0 |
| AA881388_ESTs,_Weakly_similar_to_zinc_finger_pro- | −2.2 | −1.4 | −2.4 | −1.4 |
| tein_95_[M.musculus] | | | | |
| AA881388_ESTs | −2.3 | −1.0 | −1.0 | −1.0 |
| W10293_androgen_regulated_vas_deferens_protein | −2.3 | 1.2 | −1.0 | 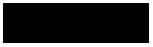 |
| AA176045_forkhead_box_C2 | −2.3 | −1.6 | −2.5 | −1.1 |
| AA546645_cell_division_cycle_2_homolog_(S._pombe)-like_2 | −2.3 | −1.5 | −2.4 | −2.4 |
| AA221794_regulator_of_G-protein_signaling_2 | −2.3 | −1.1 | −1.3 | −1.3 |
| AI451862_RIKEN_cDNA_0910001A18_gene | −2.4 | −1.3 | −1.1 | −1.3 |
| AI892132_cytochrome_c_oxidase,_subunit_VI_a,_polypeptide_2 | −2.4 | 1.0 | −1.0 | −1.4 |
| AA065510_defensin_beta_1 | −2.5 | −1.1 | −1.1 | −1.3 |
| AI386046_trypsin_4 | −2.5 | −1.1 | −1.3 | −1.2 |
| W15001_CD52_antigen | −2.5 | −1.1 | −1.1 | −1.0 |
| AA238062_CD52_antigen | −2.6 | −1.1 | −1.2 | −1.1 |
| AA237793_rat_regenerating_islet-derived,_mouse_homolog_1 | −2.7 | −1.2 | −1.2 | −1.2 |
| AA717025_Mus_musculus_10_day_old_male_pan- | −2.9 | −1.2 | −1.1 | −1.4 |
| creas_cDNA,_RIKEN_fulllength_enriched_library,_clone: | | | | |
| 1810009A17,_full_insert_sequence | | | | |
| AA771566_troponin_C,_fast_skeletal | −3.0 | −1.3 | −1.3 | −1.3 |
| AA189266_ESTs | −3.0 | −1.8 | −3.9 | −1.4 |
| AA097074_aquaporin_2 | −3.4 | −1.3 | 1.2 | 1.2 |
| AA712003_resistin_like_alpha | −3.7 | −1.1 | 1.2 | −1.4 |
| AA675084_elastase_2 | −4.0 | −1.4 | −1.2 | −1.4 |
| AA790398_ribonuclease_1,_pancreatic | −4.1 | −1.1 | −1.2 | −1.1 |
| AA821997_myoglobin | −4.3 | 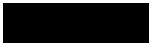 | −1.1 | −1.7 |
| AA821884_amylase_2,_pancreatic | −4.4 | −1.1 | −1.1 | −1.2 |
| AA242201_serine_protease_inhibitor,_Kazal_type_3 | −9.2 | −1.0 | 1.0 | 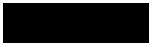 |

LUNG

| 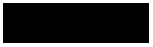 | spl | panc | liver | stom |
|---|---|---|---|---|
| Description | PL-4B_BDEnor | PL-2B_BDEnor | PL-6B_BDEnor | PL-8B_BDEnor |
| 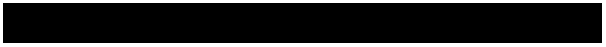 | 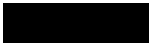 | 1.1 | 1.0 | −1.2 |
| 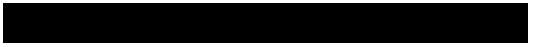 | 1.1 | −1.1 | −1.0 | 1.2 |
| 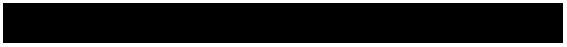 | −1.2 | 1.2 | 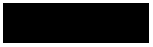 | −1.1 |
| 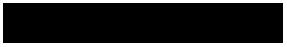 | 1.2 | 1.1 | −1.1 | −1.4 |
| 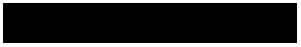 | 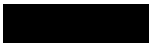 | 1.5 | −1.2 | −1.4 |
| 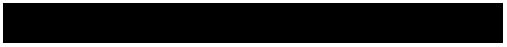 | −1.0 | −1.3 | −1.4 | −1.3 |
| 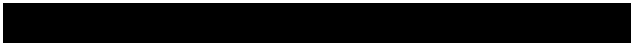 | 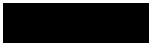 | 1.1 | −1.1 | 1.3 |
| AA623175_ESTs | 1.5 | 1.5 | −1.0 | 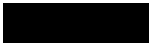 |
| AA469630_choline_kinase | 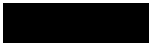 | 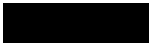 | −1.4 | 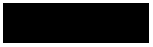 |
| AA657278_DnaJ_(Hsp40)_homolog,_subfamily_B._member_1 | 1.3 | 1.12686+ | 1.1 | 1.5 |
| AI549624_RIKEN_cDNA_0610041E09_gene | 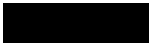 | 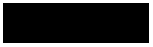 | −1.3 | 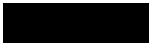 |
| AA466852_cysteine_rich_protein_61 | 1.1 | 1.1 | −1.1 | −1.1 |
| AA412879_RIKEN_cDNA_C330006J08_gene | 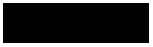 | 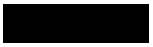 | −1.0 | 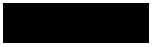 |
| AA073925_ESTs | 1.3 | 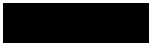 | −1.4 | 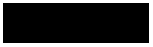 |
| AA726092_RIKEN_cDNA_4933419D20_gene | 1.1 | 1.1 | 1.4 | 1.0 |
| AI551943_ESTs,_Weakly_similar_to_JC2378_acetyl- CoA_C-acetyltransferase_[H.sapiens] | 1.2 | 1.1 | −1.4 | 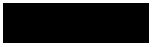 |
| AI549643_RIKEN_cDNA_1300017N15_gene | 1.3 | 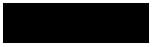 | −1.1 | 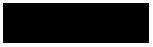 |
| AA209882_nuclear_receptor_subfamily_4,_group_A,_member_1 | −1.0 | −1.0 | −1.0 | −1.6 |
| AA062457_RIKEN_cDNA_0610006F02_gene | 1.2 | −1.1 | 1.3 | 1.1 |
| AI552795_ESTs,_Weakly_simi- | 1.1 | −1.2 | −1.1 | 1.5 |
| lar_to_NPT1_MOUSE_RENAL_SODIUM-DEPENDENT_PHOS- | | | | |
| PHATE_TRANSPORT_PROTEIN_1_[M.musculus] | | | | |
| AI325603_apolipoprotein_E | 1.2 | 1.3 | 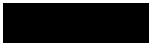 | −1.7 |
| AA789873_ATPase,_H+_transporting,_lysosomal_(vacuolar_pro- | −1.5 | 1.4 | 1.3 | 1.0 |
| ton_pump)_42kD | | | | |
| AI326605_cyclin-dependent_kinase_inhibitor_1A_(P21) | −1.1 | −1.1 | 1.2 | 1.3 |
| AI595258_elongation_of_very_long_chain_fatty_acids_(FEN1/ | −1.4 | 1.2 | 1.3 | 1.1 |
| Elo2,_SUR4/Elo3,_yeast)-like_2 | | | | |
| AA051684_apolipoprotein_A_II | 1.1 | 1.3 | −1.0 | 1.1 |
| AA790398_ribonuclease_1,_pancreatic | 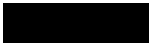 | 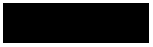 | −1.2 | −5.8 |
| AI465691_ESTs | −1.6 | 1.1 | 1.2 | 1.2 |
| AI385457_retinol_binding_protein_2,_cellular | −2.0 | −2.1 | −1.6 | −1.9 |
| AA675084_elastase_2 | 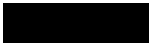 | 1.4 | −1.2 | −2.7 |
| AA824095_RIKEN_cDNA_2610205H19_gene | −1.0 | −1.2 | 1.3 | −1.9 |
| AA107035_guanylate_cyclase_activator_2b_(retina) | −1.2 | −1.3 | 1.1 | −1.1 |
| AA572640_RIKEN_cDNA_1700124F02_gene | −1.10123+ | −1.00385+ | 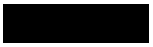 | −1.2 |
| W16266_cytochrome_c_oxidase,_subunit_VIIa_1 | −1.0 | −1.8 | −1.0 | −1.1 |
| AA106072_Public_domain_EST | 1.0 | −1.7 | 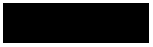 | −1.1 |
| AA572623_tryptophan_2,3-dioxygenase | −1.0 | −1.01186+ | 1.4 | 1.1 |
| AA065775_gap_junction_membrane_channel_protein_beta_1 | 1.2 | 1.1 | 1.4 | −1.5 |
| AA062408_ESTs,_Weakly_similar_to_proteasome_in- hibitor_hP131_subunit_[H.sapiens] | −1.3 | 1.2 | 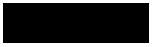 | 1.1 |
| AA617321_ESTs,_Moderately_similar_to_OXRTGU_L- | 1.01627+ | −1.07473+ | 1.1 | −1.0 |
| gulonolactone_oxidase_[R.norvegicus] | | | | |
| AA546945_tyrosine_3-monooxygenase/tryptophan_5- | −1.3 | 1.3 | 1.4 | −1.1 |
| monooxygenase_activation_protein,_zeta_polypeptide | | | | |
| AI325764_midkine | −1.2 | −1.5 | −1.5 | −1.5 |
| W08617_zinc_finger_protein_67 | −1.5 | −1.1 | 1.2 | −1.2 |
| AA437863_RIKEN_cDNA_1200013I08_gene | −1.6 | 1.0 | 1.4 | −1.4 |
| AA414078_DNA_segment,_Chr_7,_ERATO_doi_486,_expressed | −1.7 | 1.2 | 1.4 | 1.2 |
| W99034_ESTs | −1.2 | 1.3 | 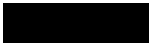 | 1.5 |
| AA667301_troponin_I,_cardiac | 1.4 | −1.6 | −1.1 | −2.1 |
| AI608086_runt_related_transcription_factor_3 | −2.0 | 1.1 | 1.2 | 1.4 |
| W56995_hemoglobin_X,_alpha- | 1.1 | −1.6 | −1.3 | −1.2 |
| like_embryonic_chain_in_Hba_complex | | | | |
| AA771393_cytochrome_P450,_2d10 | 1.2 | −1.4 | 1.3 | −1.0 |
| AI893936_hepatic_nuclear_factor_4 | 1.0 | 1.1 | 1.4 | −1.1 |
| AI536309_RIKEN_cDNA_2210410L06_gene | −1.8 | −1.8 | −1.4 | −2.1 |
| AA475351_cytochrome_P450,_steroid_inducible_3a13 | 1.1 | −1.1 | 1.4 | −1.1 |
| W62007_neuron_specific_gene_family_member_2 | −1.5 | −1.5 | −1.9 | −2.3 |
| AA066694_solute_carrier_family_27_(fatty_acid_trans- | −1.3 | −1.5 | 1.4 | 1.4 |
| porter),_member_2 | | | | |
| AA231099_necdin | −1.8 | 1.3 | 1.1 | 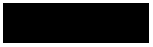 |
| AA016759_mini_chromosome_maintenance_defi- | −1.4 | 1.1 | 1.1 | 1.1 |
| cient_6_(S._cerevisiae) | | | | |
| AI508995_poliovirus_receptor-related_1 | −1.6 | 1.3 | 1.3 | −1.0 |
| AA107101_prostate_stem_cell_antigen | −1.1 | −1.4 | −1.3 | −2.1 |
| AA874101_tumor-associated_calcium_signal_transducer_2 | −2.6 | −1.1 | 1.5 | 1.3 |
| W13619_small_EDRK-rich_factor_1 | 1.1 | 1.1 | 1.0 | −1.0 |
| AA674987_RIKEN_cDNA_1110038G14_gene | −1.1 | 1.3 | −1.1 | −2.1 |
| AA822480_ESTs,_Weakly_similar_to_retinal_short- chain_dehydrogenase/reductase_retSDR1_[M.musculus] | 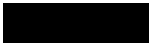 | 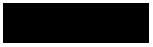 | 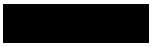 | 1.1 |
| AA067002_methionine_adenosyltransferase_I,_alpha | 1.4 | 1.3 | 1.2 | 1.3 |
| W78548_alpha_intemexin_neuronal_intermediate_filament_protein | 1.3 | −1.2 | −1.2 | 1.4 |
| AA880322_calponin_1 | −1.2 | −1.4 | −1.1 | −1.1 |
| AI323053_forkhead_box_D3 | −1.5 | 1.0 | 1.4 | 1.0 |
| AA822366_inhibitor_of_DNA_binding_4 | −2.0 | 1.3 | 1.2 | 1.5 |
| AA966993_mutS_homolog_5_(E._coli) | −1.5 | 1.3 | 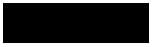 | 1.3 |
| AA096594_RIKEN_cDNA_2900089E13_gene | −1.5 | 1.1 | 1.3 | 1.4 |
| AI386062_carbonic_anhydrase_3 | 1.1 | −1.3 | 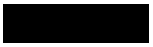 | −1.5 |
| AA880530_Public_domain_EST | −1.5 | −1.0 | 1.5 | 1.2 |
| AA049060_inter_alpha-trypsin_inhibitor,_heavy_chain_4 | 1.5 | 1.3 | 1.2 | −1.2 |
| AA571365_Mus_musculus_MRPS6_mRNA_for_mito- | −1.2 | −1.1 | 1.3 | 1.3 |
| chondrial_ribosomal_protein_S6,_partial_cds | | | | |
| AI510586_Mus_musculus_high-affinity_Na+/ | −1.3 | −1.4 | −1.1 | −1.3 |
| dicarboxylate_cotransporter_NaDC3_mRNA,_complete_cds | | | | |
| W08947_non- | −1.8 | 1.1 | 1.3 | 1.0 |
| catalytic_region_of_tyrosine_kinase_adaptor_protein_2 | | | | |
| AA755870_natriuretic_peptide_precursor_type_A | 1.1 | −1.8 | 1.2 | 1.3 |
| AA237986_cytochrome_P450,_3a25 | 1.5 | −1.2 | 1.3 | −1.1 |
| AA674821_folate_receptor_1_(adult) | −1.1 | −1.3 | 1.2 | 1.3 |
| AA755277_inter-alpha_trypsin_inhibitor,_heavy_chain_1 | 1.2 | −1.2 | 1.1 | −1.0 |
| AA867621_S-phase_kinase-associated_protein_2_(p45) | −1.8 | −1.2 | 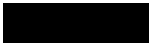 | 1.5 |
| AA066739_RIKEN_cDNA_2600001B17_gene | −1.6 | 1.3 | 1.5 | 1.0 |
| W36030_Friedreich_ataxia | −1.5 | 1.1 | 1.4 | 1.1 |
| AA066835_corticosteroid_binding_globulin | 1.0 | 1.2 | 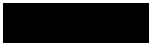 | −1.1 |
| AA674450_RIKEN_cDNA_1300007O05_gene | 1.3 | −1.8 | 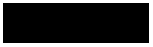 | 1.2 |
| AA881492_ESTs | −2.0 | 1.3 | 1.1 | −1.2 |
| AA242360_cytochrome_P450,_1a2,_aromatic_compound_inducible | 1.3 | −1.5 | 1.4 | −1.1 |
| AA238308_complement_component_4_binding_protein | 1.5 | −1.1 | 1.2 | −1.4 |
| AI481911_Mus_musculus,_clone_MGC:6727,_mRNA,_com- | −1.1 | −2.0 | −1.8 | 1.0 |
| plete_cds | | | | |
| W82417_apolipoprotein_CIV | 1.5 | −1.4 | 1.4 | −1.0 |
| AA242413_asialoglycoprotein_receptor_1 | 1.4 | −1.1 | 1.3 | −1.1 |
| AA066016_RIKEN_cDNA_1190002H23_gene | −1.6 | 1.2 | 1.2 | 1.5 |
| AA034678_rhotekin | −2.3 | 1.0 | 1.2 | −1.0 |
| AA763276_small_muscle_protein,_X-linked | −1.7 | −1.4 | 1.1 | 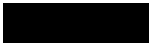 |
| AI047903_topoisomerase_(DNA)_III_beta | −1.7 | −1.0 | 1.4 | 1.1 |
| W65070_ESTs | −1.2 | 1.4 | 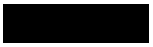 | 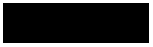 |
| AI428661_ESTs | −2.6 | −1.1 | 1.0 | −1.4 |
| AA881013_cytochrome_P450,_2c37 | 1.0 | −1.1 | 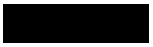 | 1.1 |
| AI322387_insulin-like_growth_factor_2 | −1.2 | −1.8 | −1.4 | −1.9 |
| AI324412_UDP-glucuronosyltransferase_2_family,_member_5 | 1.3 | −1.3 | 1.5 | 1.0 |
| AA967219_retinol_binding_protein_4,_plasma | 1.1 | −1.5 | 1.3 | 1.1 |
| W08430_serine_protease_inhibitor_1-4 | 1.3 | 1.3 | −1.2 | 1.1 |
| AA684320_activin_receptor_interacting_protein_1 | −1.9 | 1.4 | 1.3 | 1.3 |
| AA980366_vitronectin | 1.1 | 1.5 | 1.3 | 1.1 |
| AA245687_orosomucoid_2 | 1.3 | −1.4 | 1.5 | −1.8 |
| AA014727_tumor- suppressing_subchromosomal_transferable_fragment_3 | −2.1 | 1.1 | 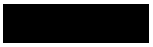 | −1.2 |
| AA546645_cell_division_cycle_2_homolog_(S._pombe)-like_2 | −2.1 | −1.1 | 1.3 | 1.4 |
| AA176045_forkhead_box_C2 | −2.5 | 1.1 | 1.4 | 1.4 |
| W33918_serine_(or_custeine)_proteinase_inhibitor,_clade_F_(alpha- 2_antiplasmin,_pigment_epithelium_derived_factor),_member_2 | 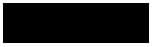 | −1.1 | 1.5 | −1.1 |
| AI326560_complement_component_factor_i | 1.3 | −1.2 | 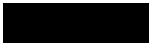 | −1.5 |
| AA771229_ESTs,_Weakly_similar_to_zinc_finger_pro- | −2.4 | 1.2 | 1.3 | 1.2 |
| tein_95_[M.musculus] | | | | |
| AA821932_small_inducible_cytokine_A6 | 1.0 | −1.0 | 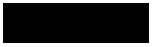 | −1.6 |
| AA212714_murinoglobulin_2 | 1.4 | −1.5 | 1.4 | −1.4 |
| AA106793_plasminogen | 1.1 | −1.6 | 1.3 | 1.0 |
| AA474336_synaptonemal_complex_protein_3 | −2.8 | 1.2 | 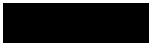 | 1.4 |
| AI893895_cytochrome_P450,_2d9 | 1.2 | −1.7 | 1.3 | −1.4 |
| AA822002_cytochrome_P450,_2c40 | 1.3 | −1.2 | 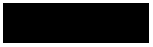 | 1.3 |
| AA733490_serine_protease_inhibitor-2_related_sequence_1 | 1.5 | −1.2 | 1.5 | 1.4 |
| W14912_kallikrein_binding_protein | 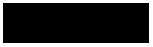 | −1.7 | 1.5 | −1.4 |
| AI117779_orosomucoid_1 | 1.1 | −1.8 | 1.3 | −1.5 |
| AA388066_regucalcin | 1.2 | −1.3 | 1.5 | −1.1 |
| AA024217_Public_domain_EST | −2.5 | 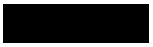 | 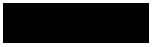 | 1.2 |
| AA261401_serum_amyloid_P-component | 1.4 | −1.3 | −1.2 | −1.4 |
| AA016788_ESTs,_Moderately_similar_to_Edp1_pro- | 1.2 | −1.0 | 1.2 | −1.0 |
| tein_[M.musculus] | | | | |
| W34349_coagulation_factor_XII_(Hageman_factor) | 1.1 | 1.1 | 1.2 | 1.1 |
| AA272831_betaine-homocysteine_methyltransferase | 1.3 | −1.3 | 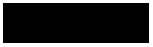 | −1.0 |
| AA822029_group_specific_component | 1.5 | −1.4 | 1.0 | 1.0 |
| AI386236_DNA_segment,_Chr_1,_Uni- versity_of_California_at_Los_Angeles_3 | −1.0 | −1.5 | 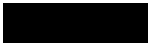 | 1.3 |
| AI466451_fatty_acid_binding_protein_1,_liver | −1.5 | −1.5 | 1.4 | −1.2 |
| AA771396_4-hydroxyphenylpyruvic_acid_dioxygenase | 1.1 | −1.4 | 1.2 | 1.1 |
| AA189266_ESTs | −3.2 | 1.4 | 1.5 | 1.5 |
| AA670733_{murinoglobulin}_2 | 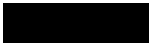 | −1.1 | 1.2 | 1.4 |
| AA980395_apolipoprotein_H | 1.3 | −1.3 | 1.4 | 1.4 |
| AA822116_transthyretin | 1.2 | −1.1 | 1.5 | −1.5 |
| AA066867_RIKEN_cDNA_3010002H13_gene | 1.3 | −1.1 | 1.0 | −1.1 |
| W54403_ATPase,_Cu++_transporting,_beta_polypeptide | 1.2 | −2.1 | 1.1 | −1.0 |
| AA675398_murihoglobulin_1 | 1.5 | −1.5 | 1.1 | −1.0 |
| AA647336_cytochrome_P450,_steroid_inducible_3a11 | 1.5 | −1.4 | 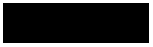 | 1.1 |
| AA822098_apolipoprotein_A-I | −1.9 | −1.5 | 1.1 | −2.4 |
| AA066397_alpha-2-macroglobulin | 1.3 | −1.4 | 1.0 | 1.1 |
| AA822009_hemopexin | 1.1 | −1.2 | −1.1 | 1.2 |
| W13979_serine_protease_inhibitor_1-5 | 1.1 | −1.7 | 1.2 | −1.3 |
| W37032_serine_protease_inhibitor_1-3 | 1.1 | −1.6 | 1.4 | −1.5 |
| AA674270_major_urinary_protein_1 | 1.1 | −1.1 | −5.01013* | −2.7 |
| AA822106_ESTs,_Weakly_similar_to_CO8A_HUMAN_COM- PLEMENT_COMPONENT_C8_ALPHA_CHAIN_PRE- CURSOR_[H.sapiens] | 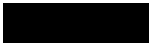 | −1.7 | 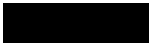 | −1.0 |
| AA822027_ATPase,_Cu++_transporting,_beta_polypeptide | 1.2 | −1.9 | 1.2 | −1.4 |
| AI386058_serum_albumin_variant | 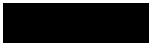 | −1.7 | 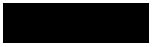 | 1.1 |
| AA822105_urinary_protein_1 | −1.1 | −2.4 | −28.7629* | −7.8 |

LUNG

| 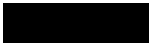 | int | col | brain | 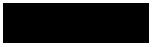 |
|---|---|---|---|---|
| Description | PL-3B_BDEnor | PL-5B_BDEnor | PL-8B_BDEnor | PL-7B_BDEnor |
| 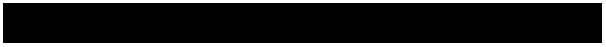 | −1.1 | −1.3 | −1.8 | 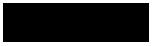 |
| 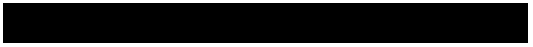 | −1.8 | −2.0 | −1.7 | 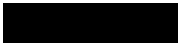 |
| 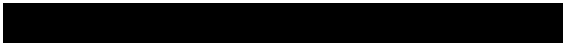 | 1.2 | −1.58765˜ | 1.5 | 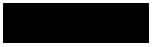 |
| 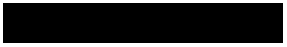 | −1.9 | −1.2 | −1.5 | 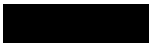 |
| 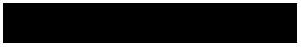 | −2.7 | −2.3 | −1.2 | 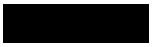 |
| 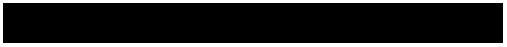 | −1.1 | −2.28344˜ | 1.3 | 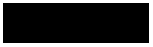 |
| 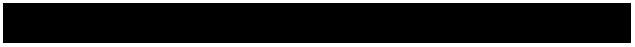 | 1.2 | −1.6 | −1.5 | 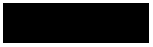 |
| AA623175_ESTs | 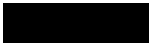 | −1.30902+˜ | 1.4 | 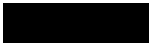 |
| AA469630_choline_kinase | 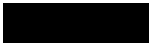 | 1.31548+˜ | 1.3 | 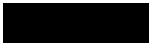 |
| AA657278_DnaJ_(Hsp40)_homolog,_subfamily_B._member_1 | 1.0 | −1.4909+˜ | 1.1 | 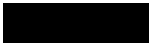 |
| AI549624_RIKEN_cDNA_0610041E09_gene | 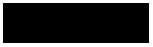 | −1.01549+˜ | 1.1 | 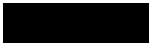 |
| AA466852_cysteine_rich_protein_61 | 1.1 | −1.03021+˜ | −1.0 | 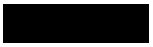 |
| AA412879_RIKEN_cDNA_C330006J08_gene | 1.2 | −1.3 | 1.3 | 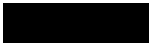 |
| AA073925_ESTs | 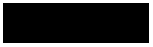 | −1.27077˜ | 1.1 | 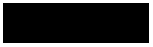 |
| AA726092_RIKEN_cDNA_4933419D20_gene | 1.5 | 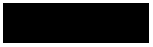 | −1.26553+ | 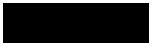 |
| AI551943_ESTs,_Weakly_similar_to_JC2378_acetyl- CoA_C-acetyltransferase_[H.sapiens] | 1.3 | 1.05496˜ | 1.4 | 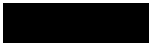 |
| AI549643_RIKEN_cDNA_1300017N15_gene | 1.4 | 1.0667˜ | 1.4 | 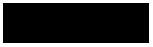 |
| AA209882_nuclear_receptor_subfamily_4,_group_A,_member_1 | −1.3 | −1.06815˜ | −1.8 | 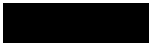 |
| AA062457_RIKEN_cDNA_0610006F02_gene | 1.1 | −1.4 | −2.3 | −1.9 |
| AI552795_ESTs,_Weakly_simi- lar_to_NPT1_MOUSE_RENAL_SODIUM-DEPENDENT_PHOS- PHATE_TRANSPORT_PROTEIN_1_[M.musculus] | 1.0 | 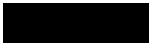 | 1.1 | −1.9 |
| AI325603_apolipoprotein_E | 1.5 | −1.0 | 1.5 | −1.9 |
| AA789873_ATPase,_H+_transporting,_lysosomal_(vacuolar_pro- ton_pump)_42kD | 1.4 | 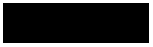 | −1.1 | −1.9 |
| AI326605_cyclin-dependent_kinase_inhibitor_1A_(P21) | 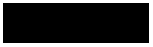 | −1.04083˜ | −1.6 | −1.9 |
| AI595258_elongation_of_very_long_chain_fatty_acids_(FEN1/ | 1.3 | 1.5 | −1.7 | −1.9 |
| Elo2,_SUR4/Elo3,_yeast)-like_2 | | | | |
| AA051684_apolipoprotein_A_II | −1.0 | −1.2 | −1.6 | −1.9 |
| AA790398_ribonuclease_1,_pancreatic | −1.6 | −3.3 | 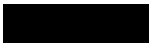 | −1.9 |
| AI465691_ESTs | 1.3 | 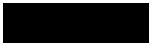 | −2.17089+ | −1.9 |
| AI385457_retinol_binding_protein_2,_cellular | 1.5 | 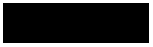 | −1.3 | −1.9 |
| AA675084_elastase_2 | −21.7 | −2.2 | 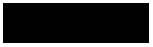 | −1.9 |
| AA824095_RIKEN_cDNA_2610205H19_gene | −1.3 | 1.2 | −1.7 | −1.9 |
| AA107035_guanylate_cyclase_activator_2b_(retina) | 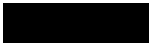 | 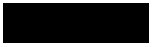 | −1.5 | −1.9 |
| AA572640_RIKEN_cDNA_1700124F02_gene | 1.2 | 1.8494+˜ | 1.26699+ | −1.9 |
| W16266_cytochrome_c_oxidase,_subunit_VIIa_1 | 1.2 | −1.2 | −1.1 | −1.9 |
| AA106072_Public_domain_EST | 1.0 | 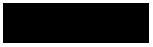 | −2.2 | −1.9 |
| AA572623_tryptophan_2,3-dioxygenase | 1.1 | 1.1083˜ | −1.25829+ | −2.0 |
| AA065775_gap_junction_membrane_channel_protein_beta_1 | −1.8 | 1.1 | −1.6 | −2.0 |
| AA062408_ESTs,_Weakly_similar_to_proteasome_in- hibitor_hP131_subunit_[H.sapiens] | 1.3 | 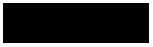 | −1.7 | −2.0 |
| AA617321_ESTs,_Moderately_similar_to_OXRTGU_L- | −1.1 | 1.11102+˜ | 1.09078+ | −2.0 |
| gulonolactone_oxidase_[R.norvegicus] | | | | |
| AA546945_tyrosine_3-monooxygenase/tryptophan_5- monooxygenase_activation_protein,_zeta_polypeptide | 1.2 | 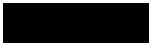 | −1.2 | −2.0 |
| AI325764_midkine | −1.2 | −1.2 | −1.1 | −2.0 |
| W08617_zinc_finger_protein_67 | 1.1 | 1.2 | −1.4 | −2.0 |
| AA437863_RIKEN_cDNA_1200013I08_gene | 1.3 | 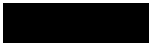 | −1.4 | −2.0 |
| AA414078_DNA_segment,_Chr_7,_ERATO_doi_486,_expressed | 1.2 | 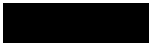 | 1.0 | −2.0 |
| W99034_ESTs | 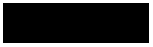 | 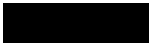 | 1.0 | −2.0 |
| AA667301_troponin_I,_cardiac | 1.3 | 1.41297˜ | −2.0 | −2.0 |
| AI608086_runt_related_transcription_factor_3 | 1.4 | 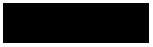 | −1.75763+ | −2.0 |
| W56995_hemoglobin_X,_alpha- | −2.2 | 1.11232˜ | −2.1 | −2.0 |
| like_embryonic_chain_in_Hba_complex | | | | |
| AA771393_cytochrome_P450,_2d10 | 1.4 | 1.0 | 1.2 | −2.0 |
| AI893936_hepatic_nuclear_factor_4 | 1.1 | −1.3 | −1.7 | −2.0 |
| AI536309_RIKEN_cDNA_2210410L06_gene | −1.9 | 1.1 | −1.5 | −2.0 |
| AA475351_cytochrome_P450,_steroid_inducible_3a13 | 1.3 | 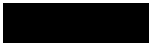 | 1.3 | −2.0 |
| W62007_neuron_specific_gene_family_member_2 | −1.3 | −1.0 | 1.4 | −2.0 |
| AA066694_solute_carrier_family_27_(fatty_acid_trans- porter),_member_2 | −1.0 | 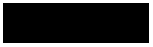 | −1.3 | −2.0 |
| AA231099_necdin | 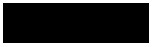 | 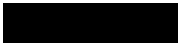 | 1.0 | −2.0 |
| AA016759_mini_chromosome_maintenance_defi- cient_6_(S._cerevisiae) | 1.3 | 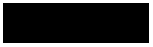 | −2.9 | −2.0 |
| AI508995_poliovirus_receptor-related_1 | 1.3 | 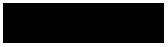 | −1.2 | −2.0 |
| AA107101_prostate_stem_cell_antigen | −6.1 | 1.51614˜ | −1.8 | −2.1 |
| AA874101_tumor-associated_calcium_signal_transducer_2 | 1.2 | 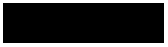 | −1.7 | −2.1 |
| W13619_small_EDRK-rich_factor_1 | −1.0 | −1.89596˜ | 1.1 | −2.1 |
| AA674987_RIKEN_cDNA_1110038G14_gene | 1.2 | 1.5 | −1.3 | −2.1 |
| AA822480_ESTs,_Weakly_similar_to_retinal_short- chain_dehydrogenase/reductase_retSDR1_[M.musculus] | −1.2 | 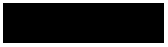 | −1.5 | −2.1 |
| AA067002_methionine_adenosyltransferase_I,_alpha | 1.3 | 1.2999˜ | −1.6 | −2.1 |
| W78548_alpha_intemexin_neuronal_intermediate_filament_protein | 1.0 | 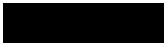 | 1.2 | −2.1 |
| AA880322_calponin_1 | 1.1 | −2.6 | −1.7 | −2.1 |
| AI323053_forkhead_box_D3 | 1.2 | 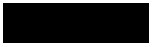 | −1.4 | −2.1 |
| AA822366_inhibitor_of_DNA_binding_4 | 1.4 | 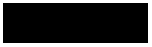 | 1.1 | −2.1 |
| AA966993_mutS_homolog_5_(E._coli) | 1.3 | 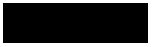 | −1.0 | −2.1 |
| AA096594_RIKEN_cDNA_2900089E13_gene | 1.1 | 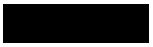 | −1.2 | −2.1 |
| AI386062_carbonic_anhydrase_3 | 1.1 | −2.4 | −1.7 | −2.1 |
| AA880530_Public_domain_EST | 1.2 | 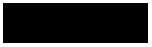 | 1.5 | −2.2 |
| AA049060_inter_alpha-trypsin_inhibitor,_heavy_chain_4 | −1.0 | 1.05302˜ | −2.1 | −2.2 |
| AA571365_Mus_musculus_MRPS6_mRNA_for_mito- chondrial_ribosomal_protein_S6,_partial_cds | 1.3 | 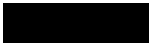 | −1.9 | −2.2 |
| AI510586_Mus_musculus_high-affinity_Na+/ dicarboxylate_cotransporter_NaDC3_mRNA,_complete_cds | −1.1 | 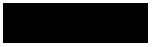 | −1.2 | −2.2 |
| W08947_non- | 1.2 | 1.4 | −2.2 | −2.2 |
| catalytic_region_of_tyrosine_kinase_adaptor_protein_2 | | | | |
| AA755870_natriuretic_peptide_precursor_type_A | −1.1 | 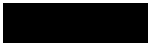 | −1.3 | −2.2 |
| AA237986_cytochrome_P450,_3a25 | 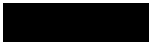 | 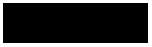 | −1.1 | −2.2 |
| AA674821_folate_receptor_1_(adult) | 1.1 | 1.5 | −1.2 | −2.2 |
| AA755277_inter-alpha_trypsin_inhibitor,_heavy_chain_1 | 1.3 | 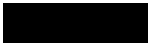 | −1.96147+ | −2.2 |
| AA867621_S-phase_kinase-associated_protein_2_(p45) | 1.2 | 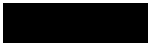 | −1.73102+ | −2.2 |
| AA066739_RIKEN_cDNA_2600001B17_gene | 1.3 | 1.2 | −1.4 | −2.2 |
| W36030_Friedreich_ataxia | 1.2 | 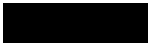 | −1.6 | −2.3 |
| AA066835_corticosteroid_binding_globulin | 1.2 | 1.4 | −2.4 | −2.3 |
| AA674450_RIKEN_cDNA_1300007O05_gene | 1.3 | 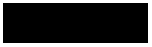 | −1.2 | −2.4 |
| AA881492_ESTs | 1.1 | 1.1 | 1.0 | −2.4 |
| AA242360_cytochrome_P450,_1a2,_aromatic_compound_inducible | −2.8 | −1.33495˜ | −1.37353+ | −2.4 |
| AA238308_complement_component_4_binding_protein | 1.2 | −1.42054˜ | −1.1 | −2.4 |
| AI481911_Mus_musculus,_clone_MGC:6727,_mRNA,_com- | −1.8 | −2.3 | −1.5 | −2.4 |
| plete_cds | | | | |
| W82417_apolipoprotein_CIV | −1.0 | −1.16601˜ | −1.64355+ | −2.4 |
| AA242413_asialoglycoprotein_receptor_1 | 1.0 | −1.17665+˜ | −1.2 | −2.4 |
| AA066016_RIKEN_cDNA_1190002H23_gene | 1.5 | 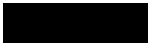 | −2.0 | −2.4 |
| AA034678_rhotekin | 1.2 | 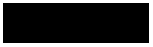 | −1.3 | −2.5 |
| AA763276_small_muscle_protein,_X-linked | 1.2 | 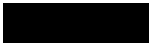 | −4.6 | −2.5 |
| AI047903_topoisomerase_(DNA)_III_beta | 1.1 | 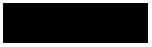 | −1.4 | −2.5 |
| W65070_ESTs | 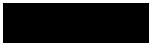 | 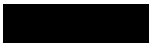 | 1.1 | −2.6 |
| AI428661_ESTs | 1.2 | 1.2 | −1.6 | −2.6 |
| AA881013_cytochrome_P450,_2c37 | 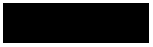 | 1.5 | −1.2 | −2.6 |
| AI322387_insulin-like_growth_factor_2 | −1.5 | 1.4 | −1.5 | −2.6 |
| AI324412_UDP-glucuronosyltransferase_2_family,_member_5 | −1.1 | 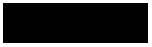 | −1.1 | −2.7 |
| AA967219_retinol_binding_protein_4,_plasma | 1.1 | 1.3 | −1.8 | −2.7 |
| W08430_serine_protease_inhibitor_1-4 | −1.2 | −1.8 | 1.1 | −2.7 |
| AA684320_activin_receptor_interacting_protein_1 | 1.5 | 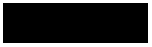 | 1.0 | −2.7 |
| AA980366_vitronectin | −1.1 | 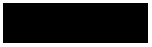 | 1.2 | −2.9 |
| AA245687_orosomucoid_2 | −1.4 | −1.70975˜ | −1.6 | −3.0 |
| AA014727_tumor- suppressing_subchromosomal_transferable_fragment_3 | 1.1 | 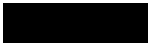 | −1.8 | −3.0 |
| AA546645_cell_division_cycle_2_homolog_(S._pombe)-like_2 | 1.1 | 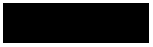 | −1.5 | −3.0 |
| AA176045_forkhead_box_C2 | 1.1 | 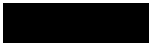 | −1.7 | −3.0 |
| W33918_serine_(or_custeine)_proteinase_inhibitor,_clade_F_(alpha- | −1.1 | −1.36639˜ | 1.2 | −3.1 |
| 2_antiplasmin,_pigment_epithelium_derived_factor),_member_2 | | | | |
| AI326560_complement_component_factor_i | −1.6 | 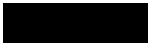 | 1.2 | −3.1 |
| AA771229_ESTs,_Weakly_similar_to_zinc_finger_pro- tein_95_[M.musculus] | 1.2 | 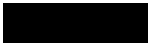 | −2.0 | −3.3 |
| AA821932_small_inducible_cytokine_A6 | 1.3 | 1.2 | −1.3 | −3.4 |
| AA212714_murinoglobulin_2 | −1.1 | −1.0114˜ | −1.3 | −3.4 |
| AA106793_plasminogen | 1.1 | 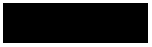 | −2.2 | −3.6 |
| AA474336_synaptonemal_complex_protein_3 | 1.5 | 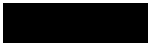 | 1.1 | −3.7 |
| AI893895_cytochrome_P450,_2d9 | 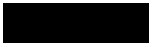 | 1.1 | −1.9 | −3.7 |
| AA822002_cytochrome_P450,_2c40 | 1.4 | 1.4 | 1.1 | −3.9 |
| AA733490_serine_protease_inhibitor-2_related_sequence_1 | 1.3 | 1.2 | 1.2 | −3.9 |
| W14912_kallikrein_binding_protein | −1.2 | −1.3 | 1.2 | −4.0 |
| AI117779_orosomucoid_1 | −1.3 | −1.64866˜ | −1.8 | −4.2 |
| AA388066_regucalcin | 1.2 | 1.00609˜ | −1.2 | −4.2 |
| AA024217_Public_domain_EST | 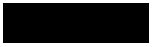 | 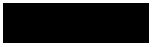 | −1.0 | −4.2 |
| AA261401_serum_amyloid_P-component | −1.0 | −1.65106˜ | 1.0 | −4.3 |
| AA016788_ESTs,_Moderately_similar_to_Edp1_pro- | 1.3 | 1.47097˜ | −1.4 | −4.5 |
| tein_[M.musculus] | | | | |
| W34349_coagulation_factor_XII_(Hageman_factor) | 1.1 | 1.07468˜ | 1.1 | −4.6 |
| AA272831_betaine-homocysteine_methyltransferase | 1.1 | −1.62438˜ | 1.1 | −4.7 |
| AA822029_group_specific_component | 1.3 | 1.5 | −1.4 | −5.1 |
| AI386236_DNA_segment,_Chr_1,_Uni- | −1.5 | 1.3 | −1.1 | −5.1 |
| versity_of_California_at_Los_Angeles_3 | | | | |
| AI466451_fatty_acid_binding_protein_1,_liver | −1.2 | 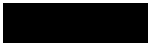 | −1.3 | −5.1 |
| AA771396_4-hydroxyphenylpyruvic_acid_dioxygenase | 1.4 | 1.1 | −2.1 | −5.2 |
| AA189266_ESTs | 1.3 | 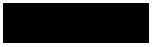 | −1.3 | −5.6 |
| AA670733_{murinoglobulin}_2 | 1.3 | 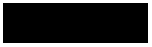 | −1.4 | −5.8 |
| AA980395_apolipoprotein_H | 1.2 | 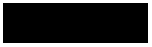 | −1.0 | −7.1 |
| AA822116_transthyretin | −1.4 | 1.55627˜ | −1.3 | −7.7 |
| AA066687_RIKEN_cDNA_3010002H13_gene | −2.0 | 1.48546˜ | 1.4 | −7.9 |
| W54403_ATPase,_Cu++_transporting,_beta_polypeptide | 1.2 | 1.3 | −2.9 | −7.9 |
| AA675398_murihoglobulin_1 | 1.0 | 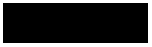 | −1.8 | −7.9 |
| AA647336_cytochrome_P450,_steroid_inducible_3a11 | 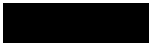 | 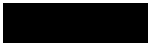 | −1.3 | −8.0 |
| AA822098_apolipoprotein_A-I | 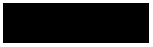 | 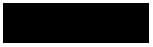 | −1.9 | −9.0 |
| AA066397_alpha-2-macroglobulin | 1.2 | 1.52434˜ | 1.1 | −10.5 |
| AA822009_hemopexin | 1.1 | 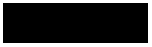 | −1.6 | −11.1 |
| W13979_serine_protease_inhibitor_1-5 | −1.3 | −1.1 | −1.5 | −14.7 |
| W37032_serine_protease_inhibitor_1-3 | −1.5 | −1.11273˜ | −1.3 | −15.2 |
| AA674270_major_urinary_protein_1 | 1.0 | 1.09942˜ | 1.5 | −16.7 |
| AA822106_ESTs,_Weakly_similar_to_CO8A_HUMAN_COM- PLEMENT_COMPONENT_C8_ALPHA_CHAIN_PRE- CURSOR_[H.sapiens] | 1.1 | 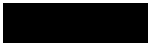 | −1.8 | −20.2 |
| AA822027_ATPase,_Cu++_transporting,_beta_polypeptide | −1.0 | 1.17594˜ | −1.2 | −21.9 |
| AI386058_serum_albumin_variant | −1.1 | 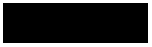 | −1.3 | −23.1 |
| AA822105_urinary_protein_1 | −1.1 | 1.43224˜ | −1.3 | −71.1 |

LUNG

| 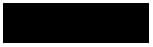 | blad | kidn | pl/ut | mam |
|---|---|---|---|---|
| Description | PL-9B_BDEnor | PL-10B_BDEnor | PL-11B_BDEnor | PL-12B_BDEnor |
| 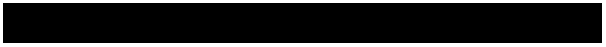 | −1.0 | 1.1 | −1.2 | 1.1 |
| 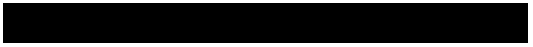 | 1.1 | 1.1 | −1.0 | −1.1 |
| 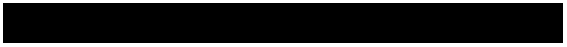 | 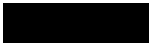 | 1.1 | −1.4 | −1.8 |
| 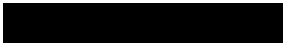 | 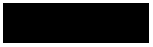 | 1.1 | −1.1 | −1.4 |
| 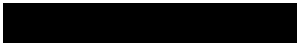 | 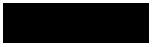 | 1.1 | 1.0 | 1.3 |
| 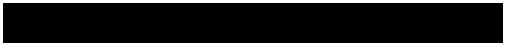 | 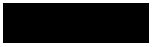 | 1.0 | −1.1 | −1.0 |
| 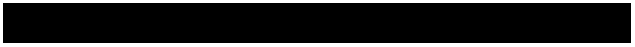 | 1.2 | 1.2 | 1.4 | 1.5 |
| AA623175_ESTs | 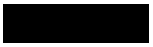 | 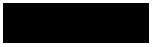 | 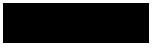 | 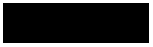 |
| AA469630_choline_kinase | 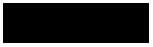 | 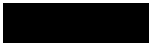 | 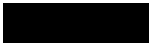 | 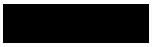 |
| AA657278_DnaJ_(Hsp40)_homolog,_subfamily_B._member_1 | 1.4 1.2 | 1.3 | 1.4 | |
| AI549624_RIKEN_cDNA_0610041E09_gene | 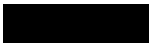 | 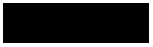 | 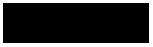 | 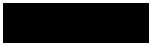 |
| AA466852_cysteine_rich_protein_61 | 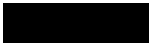 | −1.0 | 1.1 | 1.2 |
| AA412879_RIKEN_cDNA_C330006J08_gene | 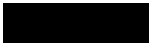 | 1.5 | 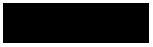 | 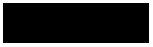 |
| AA073925_ESTs | 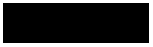 | 1.2 | 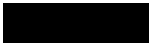 | 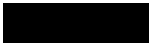 |
| AA726092_RIKEN_cDNA_4933419D20_gene | 1.3 | 1.5 | 1.1 | −1.1 |
| AI551943_ESTs,_Weakly_similar_to_JC2378_acetyl- CoA_C-acetyltransferase_[H.sapiens] | 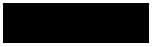 | 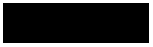 | 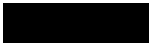 | 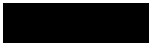 |
| AI549643_RIKEN_cDNA_1300017N15_gene | 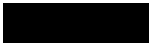 | 1.4 | 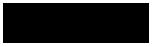 | 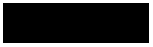 |
| AA209882_nuclear_receptor_subfamily_4,_group_A,_member_1 | 1.3 | −1.0 | 1.1 | −1.1 |
| AA062457_RIKEN_cDNA_0610006F02_gene | 1.1 | −1.3 | −1.1 | 1.3 |
| AI552795_ESTs,_Weakly_simi- | −1.3 | 1.5 | −1.1 | −1.3 |
| lar_to_NPT1_MOUSE_RENAL_SODIUM-DEPENDENT_PHOS- | | | | |
| PHATE_TRANSPORT_PROTEIN_1_[M.musculus] | | | | |
| AI325603_apolipoprotein_E | −1.3 | −1.5 | −2.0 | −1.2 |
| AA789873_ATPase,_H+_transporting,_lysosomal_(vacuolar_pro- | −1.5 | −1.0 | −1.9 | −1.2 |
| ton_pump)_42kD | | | | |
| AI326605_cyclin-dependent_kinase_inhibitor_1A_(P21) | −1.5 | −1.3 | −1.0 | 1.2 |
| AI595258_elongation_of_very_long_chain_fatty_acids_(FEN1/ | −1.7 | −1.0 | −1.7 | −1.3 |
| Elo2,_SUR4/Elo3,_yeast)-like_2 | | | | |
| AA051684_apolipoprotein_A_II | 1.1 | −1.0 | −1.0 | 1.3 |
| AA790398_ribonuclease_1,_pancreatic | −4.1 | −1.1 | −1.2 | −1.1 |
| AI465691_ESTs | −1.6 | −1.2 | −1.5 | −1.5 |
| AI385457_retinol_binding_protein_2,_cellular | −1.6 | −1.9 | −1.5 | −1.7 |
| AA675084_elastase_2 | −4.0 | −1.4 | −1.2 | −1.4 |
| AA824095_RIKEN_cDNA_2610205H19_gene | −1.1 | 1.0 | −1.4 | −1.4 |
| AA107035_guanylate_cyclase_activator_2b_(retina) | −1.2 | 1.2 | −1.3 | 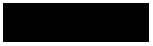 |
| AA572640_RIKEN_cDNA_1700124F02_gene | −1.3 | −1.0 | −1.2 | −1.2 |
| W16266_cytochrome_c_oxidase,_subunit_VIIa_1 | −1.5 | −1.1 | 1.0 | −1.8 |
| AA106072_Public_domain_EST | 1.0 | 1.3 | −1.3 | 1.1 |
| AA572623_tryptophan_2,3-dioxygenase | −1.1 | 1.1 | −1.1 | −1.1 |
| AA065775_gap_junction_membrane_channel_protein_beta_1 | −1.2 | 1.3 | −1.3 | −1.1 |
| AA062408_ESTs,_Weakly_similar_to_proteasome_in- | −1.3 | −1.2 | −1.5 | −1.1 |
| hibitor_hP131_subunit_[H.sapiens] | | | | |
| AA617321_ESTs,_Moderately_similar_to_OXRTGU_L- | −1.0 | −1.0 | 1.3 | 1.0 |
| gulonolactone_oxidase_[R.norvegicus] | | | | |
| AA546945_tyrosine_3-monooxygenase/tryptophan_5- | −1.5 | −1.2 | −1.8 | −1.1 |
| monooxygenase_activation_protein,_zeta_polypeptide | | | | |
| AI325764_midkine | −1.2 | −1.2 | −1.3 | −1.4 |
| W08617_zinc_finger_protein_67 | −1.7 | −1.3 | −1.8 | −1.4 |
| AA437863_RIKEN_cDNA_1200013I08_gene | −1.7 | −1.1 | −2.0 | −1.3 |
| AA414078_DNA_segment,_Chr_7,_ERATO_doi_486,_expressed | −1.4 | −1.1 | −2.5 | −1.2 |
| W99034_ESTs | −1.4 | −1.3 | −2.0 | 1.0 |
| AA667301_troponin_I,_cardiac | −2.2 | 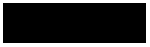 | −1.0 | 1.3 |
| AI608086_runt_related_transcription_factor_3 | −1.6 | −1.3 | −1.9 | −1.0 |
| W56995_hemoglobin_X,_alpha- | 1.0 | −1.2 | −4.0 | −1.6 |
| like_embryonic_chain_in_Hba_complex | | | | |
| AA771393_cytochrome_P450,_2d10 | 1.2 | 1.1 | −1.1 | −1.0 |
| AI893936_hepatic_nuclear_factor_4 | 1.3 | 1.2 | −1.1 | 1.2 |
| AI536309_RIKEN_cDNA_2210410L06_gene | −1.6 | −2.1 | −1.5 | −2.1 |
| AA475351_cytochrome_P450,_steroid_inducible_3a13 | 1.1 | 1.0 | 1.3 | 1.1 |
| W62007_neuron_specific_gene_family_member_2 | −1.6 | −1.4 | −1.3 | −1.5 |
| AA066694_solute_carrier_family_27_(fatty_acid_trans- | −1.1 | 1.2 | 1.1 | 1.1 |
| porter),_member_2 | | | | |
| AA231099_necdin | −1.3 | −1.2 | −1.7 | 1.0 |
| AA016759_mini_chromosome_maintenance_defi- | −1.4 | −1.3 | −1.6 | 1.1 |
| cient_6_(S._cerevisiae) | | | | |
| AI508995_poliovirus_receptor-related_1 | −1.5 | −1.4 | −1.7 | −1.3 |
| AA107101_prostate_stem_cell_antigen | −1.0 | −1.1 | −1.4 | −1.4 |
| AA874101_tumor-associated_calcium_signal_transducer_2 | −1.5 | −1.5 | −2.4 | −1.2 |
| W13619_small_EDRK-rich_factor_1 | −1.1 | −1.1 | −1.1 | −1.3 |
| AA674987_RIKEN_cDNA_1110038G14_gene | −1.4 | −1.4 | −1.8 | −1.2 |
| AA822480_ESTs,_Weakly_similar_to_retinal_short- | 1.2 | −1.2 | 1.0 | −1.7 |
| chain_dehydrogenase/reductase_retSDR1_[M.musculus] | | | | |
| AA067002_methionine_adenosyltransferase_I,_alpha | 1.3 | 1.1 | 1.2 | 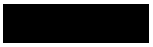 |
| W78548_alpha_intemexin_neuronal_intermediate_filament_protein | −1.2 | −1.4 | −1.4 | −1.6 |
| AA880322_calponin_1 | −1.5 | −1.2 | −1.4 | −1.0 |
| AI323053_forkhead_box_D3 | −1.5 | −1.3 | −1.7 | −1.3 |
| AA822366_inhibitor_of_DNA_binding_4 | −1.1 −1.5 | −1.6 | −1.2 | |
| AA966993_mutS_homolog_5_(E._coli) | −1.4 | −1.3 | −1.8 | −1.0 |
| AA096594_RIKEN_cDNA_2900089E13_gene | −1.3 | −1.2 | −1.8 | −1.3 |
| AI386062_carbonic_anhydrase_3 | −1.3 | −1.2 | −1.6 | −1.5 |
| AA880530_Public_domain_EST | −1.1 | −1.2 | −2.2 | −1.1 |
| AA049060_inter_alpha-trypsin_inhibitor,_heavy_chain_4 | 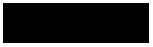 | 1.1 | 1.4 | 1.3 |
| AA571365_Mus_musculus_MRPS6_mRNA_for_mito- | −1.2 | −1.0 | −1.4 | −1.3 |
| chondrial_ribosomal_protein_S6,_partial_cds | | | | |
| AI510586_Mus_musculus_high-affinity_Na+/ | −1.3 | 1.2 | −1.4 | −1.2 |
| dicarboxylate_cotransporter_NaDC3_mRNA,_complete_cds | | | | |
| W08947_non- | −1.8 | −1.3 | −1.9 | −1.2 |
| catalytic_region_of_tyrosine_kinase_adaptor_protein_2 | | | | |
| AA755870_natriuretic_peptide_precursor_type_A | −1.8 | 1.2 | −1.4 | −1.7 |
| AA237986_cytochrome_P450,_3a25 | 1.2 | 1.1 | 1.0 | 1.3 |
| AA674821_folate_receptor_1_(adult) | −1.4 | 1.2 | −1.2 | 1.3 |
| AA755277_inter-alpha_trypsin_inhibitor,_heavy_chain_1 | 1.2 | −1.3 | −1.0 | −1.1 |
| AA867621_S-phase_kinase-associated_protein_2_(p45) | −1.8 | −1.4 | −1.9 | −1.3 |
| AA066739_RIKEN_cDNA_2600001B17_gene | −1.5 | −1.3 | −1.8 | −1.2 |
| W36030_Friedreich_ataxia | −1.5 | −1.4 | −1.7 | −1.3 |
| AA066835_corticosteroid_binding_globulin | 1.1 | −1.0 | −1.3 | −2.4 |
| AA674450_RIKEN_cDNA_1300007O05_gene | 1.1 | −1.1 | −1.0 | −1.1 |
| AA881492_ESTs | −1.5 | −1.5 | −1.7 | −1.1 |
| AA242360_cytochrome_P450,_1a2,_aromatic_compound_inducible | 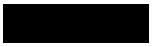 | 1.1 | 1.2 | 1.2 |
| AA238308_complement_component_4_binding_protein | 1.4 | −1.3 | 1.1 | 1.0 |
| AI481911_Mus_musculus,_clone_MGC:6727,_mRNA,_com- plete_cds | −1.4 | 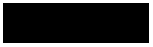 | −1.4 | −1.4 |
| W82417_apolipoprotein_CIV | −1.1 | −1.2 | −1.1 | −1.4 |
| AA242413_asialoglycoprotein_receptor_1 | 1.1 | 1.1 | 1.1 | 1.1 |
| AA066016_RIKEN_cDNA_1190002H23_gene | −1.4 | −1.6 | −2.0 | −1.3 |
| AA034678_rhotekin | −2.1 | −1.4 | −2.6 | −1.4 |
| AA763276_small_muscle_protein,_X-linked | −1.6 | −1.2 | −2.0 | −1.5 |
| AI047903_topoisomerase_(DNA)_III_beta | −1.7 | −1.5 | −2.1 | −1.5 |
| W65070_ESTs | −1.4 | −1.4 | −2.3 | −1.1 |
| AI428661_ESTs | −2.1 | −1.7 | −2.2 | −1.5 |
| AA881013_cytochrome_P450,_2c37 | 1.0 | 1.1 | −1.1 | 1.5 |
| AI322387_insulin-like_growth_factor_2 | −1.2 | −1.6 | −1.2 | −1.8 |
| AI324412_UDP-glucuronosyltransferase_2_family,_member_5 | 1.0 | −1.5 | −1.1 | 1.1 |
| AA967219_retinol_binding_protein_4,_plasma | 1.0 | −1.2 | −1.6 | −1.5 |
| W08430_serine_protease_inhibitor_1-4 | 1.0 | 1.1 | 1.2 | 1.2 |
| AA684320_activin_receptor_interacting_protein_1 | −1.4 | −1.1 | −1.9 | −1.3 |
| AA980366_vitronectin | −1.1 | −1.3 | −1.4 | −1.3 |
| AA245687_orosomucoid_2 | −1.2 | −1.2 | 1.4 | −1.6 |
| AA014727_tumor- | −2.1 | −1.8 | −2.9 | −1.0 |
| suppressing_subchromosomal_transferable_fragment_3 | | | | |
| AA546645_cell_division_cycle_2_homolog_(S._pombe)-like_2 | −2.3 | −1.5 | −2.4 | −1.4 |
| AA176045_forkhead_box_C2 | −2.3 | −1.6 | −2.5 | −1.1 |
| W33918_serine_(or_custeine)_proteinase_inhibitor,_clade_F_(alpha- | 1.1 | 1.4 | −1.6 | −2.2 |
| 2_antiplasmin,_pigment_epithelium_derived_factor),_member_2 | | | | |
| AI326560_complement_component_factor_i | 1.1 | −1.0 | −1.5 | −1.1 |
| AA771229_ESTs,_Weakly_similar_to_zinc_finger_pro- | −2.2 | −1.4 | −2.4 | −1.4 |
| tein_95_[M.musculus] | | | | |
| AA821932_small_inducible_cytokine_A6 | −1.2 | 1.3 | −1.9 | −1.7 |
| AA212714_murinoglobulin_2 | −1.0 | 1.2 | 1.5 | 1.4 |
| AA106793_plasminogen | 1.0 | 1.0 | −1.4 | −1.1 |
| AA474336_synaptonemal_complex_protein_3 | −2.1 | −1.4 | −2.3 | −1.3 |
| AI893895_cytochrome_P450,_2d9 | 1.1 | −1.0 | 1.1 | 1.1 |
| AA822002_cytochrome_P450,_2c40 | −1.1 | −1.2 | −1.3 | −1.2 |
| AA733490_serine_protease_inhibitor-2_related_sequence_1 | 1.1 | −1.1 | 1.1 | 1.1 |
| W14912_kallikrein_binding_protein | −1.0 | 1.0 | −1.1 | −1.2 |
| AI117779_orosomucoid_1 | −1.4 | −1.2 | 1.2 | −1.5 |
| AA388066_regucalcin | 1.0 | 1.0 | 1.1 | −1.2 |
| AA024217_Public_domain_EST | −1.9 | −1.2 −2.3 | 1.1 | |
| AA261401_serum_amyloid_P-component | 1.0 | 1.1 | 1.0 | 1.3 |
| AA016788_ESTs,_Moderately_similar_to_Edp1_pro- | −1.1 | −1.6 | −1.1 | −1.3 |
| tein_[M.musculus] | | | | |
| W34349_coagulation_factor_XII_(Hageman_factor) | −1.3 | −1.1 | −1.2 | 1.2 |
| AA272831_betaine_homocysteine_methyltransferase | 1.3 | −1.3 | 1.2 | −1.1 |
| AA822029_group_specific_component | 1.2 | 1.2 | −1.1 | 1.1 |
| AI386236_DNA_segment,_Chr_1,_Uni- | −1.4 | −1.2 | −1.2 | −1.5 |
| versity_of_California_at_Los_Angeles_3 | | | | |
| AI466451_fatty_acid_binding_protein_1,_liver | −1.3 | −1.2 | −1.3 | −1.2 |
| AA771396_4-hydroxyphenylpyruvic_acid_dioxygenase | 1.3 | −1.2 | 1.1 | 1.5 |
| AA189266_ESTs | −3.0 | −1.8 | −3.9 | −1.4 |
| AA670733_{murinoglobulin}_2 | −1.2 | 1.0 | 1.1 | −1.2 |
| AA980395_apolipoprotein_H | 1.3 | 1.2 | 1.1 | 1.1 |
| AA822116_transthyretin | −1.0 | 1.1 | −2.0 | −1.6 |
| AA066867_RIKEN_cDNA_3010002H13_gene | −1.1 | −1.1 | −1.7 | −1.2 |
| W54403_ATPase,_Cu++_transporting,_beta_polypeptide | 1.1 | −1.1 | −1.1 | 1.2 |
| AA675398_murihoglobulin_1 | −1.1 | −1.1 | 1.3 | 1.0 |
| AA647336_cytochrome_P450,_steroid_inducible_3a11 | −1.0 | −1.1 | 1.2 | 1.1 |
| AA822098_apolipoprotein_A-I | −1.2 | −2.1 | −2.5 | −1.6 |
| AA066397_alpha-2-macroglobulin | −1.1 | −1.7 | 1.3 | −1.2 |
| AA822009_hemopexin | 1.2 | −1.6 | −1.3 | 1.2 |
| W13979_serine_protease_inhibitor_1-5 | −1.2 | −1.1 | −1.4 | −1.5 |
| W37032_serine_protease_inhibitor_1-3 | −1.4 | −1.4 | −1.5 | −1.8 |
| AA674270_major_urinary_protein_1 | −1.5 | −1.6 | −1.6 | −1.4 |
| AA822106_ESTs,_Weakly_similar_to_CO8A_HUMAN_COM- | −1.5 | −1.3 | −1.4 | −2.7 |
| PLEMENT_COMPONENT_C8_ALPHA_CHAIN_PRE- | | | | |
| CURSOR_[H.sapiens] | | | | |
| AA822027_ATPase,_Cu++_transporting,_beta_polypeptide | −1.5 | −1.5 | −1.8 | −1.7 |
| AI386058_serum_albumin_variant | −1.4 | −1.5 | −1.3 | −2.9 |
| AA822105_urinary_protein_1 | −1.6 | −2.2 | −1.1 | −1.2 |

BRAIN

| 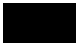 | spl | panc | liv | stom |
|---|---|---|---|---|
| Description | PL-4B_BDEnor | PL-2B_BDEnor | PL-6B_BDEnor | PL-8B_BDEnor |
| 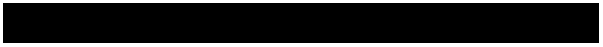 | 1.3 | −1.2 | 1.4 | −4.0 |
| 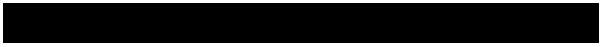 | 1.5 | −1.2 | 1.2 | −2.9 |
| 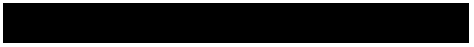 | −1.0 | −1.0 | 1.4 | −3.2 |
| 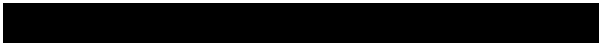 | 1.3 | −1.4 | 1.2 | −3.0 |
| 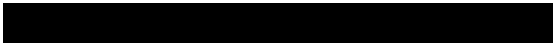 | 1.3 | 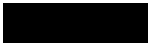 | 1.1 | 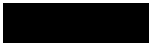 |
| 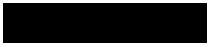 | 1.1 | 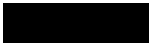 | 1.0557+ | 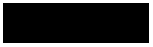 |
| 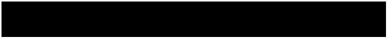 | 1.2 | 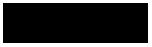 | −1.05799+ | 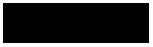 |
| 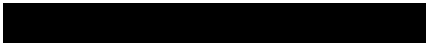 | −1.5 | 1.2 | −1.6 | 1.3 |
| AA821884_amylase_2,_pancreatic | 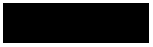 | 1.1 | −1.2 | −2.8 |
| AA588976_ESTs | 1.1 | 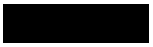 | −1.5 | 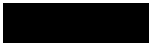 |
| W57281_Mest-linked_imprinted_transcript_1 | 1.3 | 1.2 | 1.1 | 1.3 |
| AI549687_RIKEN_cDNA_5730406I15_gene | 1.3 | 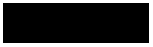 | −1.0 | 1.1 |
| AA002994_ESTs,_Moderately_similar_to_No_simi- larities_to_any_reported_proteins_[H.sapiens] | 1.1 | 1.0 | −1.3 | 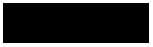 |
| AI529513_ESTs,_Moderately_similar_to_F26L_MOUSE_6PF- | −1.2 | 1.1 | 1.1 | −1.6 |
| 2-K/FRU-2,6-P2ASE_LIVER_ISOZYME_[M.musculus] | | | | |
| AI605734_septin_3 | 1.0 | −1.4 | 1.0 | 1.2 |
| AI464603_RIKEN_cDNA_0710001E13_gene | 1.1 | 1.1 | 1.0 | 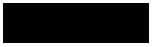 |
| AA717025_Mus_musculus_10_day_old_male_pan- crease_cDNA_RIKEN_full-length_enriched_library,_clone: 1810009A17,_full_insert_sequence | 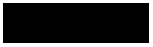 | −1.1 | −1.2 | −2.7 |
| AA117355_RIKEN_cDNA_1190006L01_gene | −1.0 | 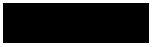 | −1.0 | 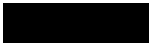 |
| AA267673_ESTs | −1.18124+ | 1.5 | 1.37787+ | 1.3 |
| AI594945_RIKEN_cDNA_6330406P08_gene | 1.05631+ | 1.20985+ | −1.07584+ | 1.2 |
| AA119984_liver-specific_bHLH-Zip_transcription_factor | 1.5 | 1.3 | 1.0 | −1.0 |
| AA522311_RIKEN_cDNA_0610038P07_gene | 1.2 | 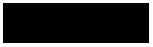 | −1.1 | 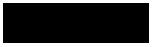 |
| AA967857_brain_protein | 1.0 | −1.0 | −1.1 | −1.2 |
| AA253844_ESTs | 1.4 | 1.3 | 1.0 | 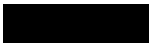 |
| AI122516_microtubule-associated_protein_6 | 1.1 | 1.0 | −1.6 | 1.4 |
| AI430438_RIKEN_cDNA_3110023G01_gene | 1.0 | 1.2 | −1.4 | 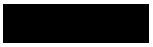 |
| AI550596_stearoyl-Coenzyme_A_desaturase_2 | 1.4 | 1.2 | 1.3 | 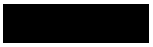 |
| AA200457_ESTs | −1.4 | 1.2 | −1.6 | −1.1 |
| AI324398_glycosylphosphatidylinositol_specific_phospholipase_D1 | 1.1 | −1.3 | 1.2 | −3.4 |
| AA929696_peptidyl_arginine_deiminase,_type_II | −1.4 | 1.0 | −1.5 | −1.3 |
| AA123314_ESTs,_Highly_similar_to_S68176_TOG_pro- | 1.1 | −1.0 | —1.2 | −1.0 |
| tein_[H.sapiens] | | | | |
| AA473684_RIKEN_cDNA_C030002O17_gene | 1.22138+ | 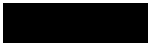 | −1.02182+ | 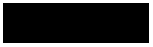 |
| AA518686_ESTs | 1.2 | 1.4 | −1.3 | 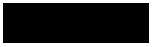 |
| AA412921_RIKEN_cDNA_4921514I20_gene | 1.4 | 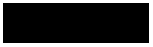 | 1.0 | 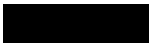 |
| AA051041_potassium_voltage- gated_channel,_subfamily_Q,_member_2 | 1.1551+ | 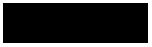 | 1.21255+ | −1.0 |
| AI604587_ESTs | 1.2 | 1.3 | −1.1 | 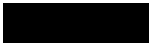 |
| AA059783_Public_domain_EST | 1.14965+ | −1.05364+ | 1.39958+ | 1.1 |
| AA466153_ESTs | 1.0 | −1.11378+ | 1.02861+ | 1.2 |
| AA222952_zuotin_related_factor_2 | 1.0 | −1.16406+ | 1.12995+ | −1.34277+ |
| AI552958_RIKEN_cDNA_1110003E08_gene | 1.0 | 1.0 | −1.1 | 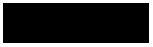 |
| AA416143_ESTs | 1.08986+ | 1.2 | −1.0032+ | 1.4 |
| AI558253_RIKEN_cDNA_1500015J03_gene | 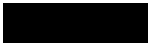 | −1.04077+ | 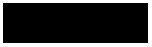 | |
| AI553232_ribosomal_protein_S6 | 1.2 | 1.5 | 1.0 | 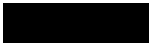 |
| AA511234_ESTs | −1.1 | 1.1 | −1.3 | 1.1 |
| AI550872_RAB11a,_member_RAS_oncogene_family | −1.1 | 1.3 | −1.6 | 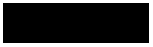 |
| AA839604_ESTs | −1.6 | −1.1 | −1.55245* | −1.2 |
| AA120302_ESTs | −1.2 | 1.1 | −1.2 | 1.3 |
| AA412912_ESTs | 1.2 | −1.5 | −1.4 | 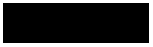 |
| AA003144_ESTs | −1.00111+ | −1.05949+ | 1.0939+ | 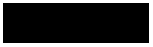 |
| AA387339_ESTs | −1.2 | 1.0 | 1.1 | −1.5 |
| AA738833_solute_carrier_family_1,_member_7 | 1.2 | −1.2 | −1.5 | −1.7 |
| AI893895_cytochrome_P450,_2d9 | 1.2 | −1.7 | 1.3 | −1.4 |
| W30045_eukaryotic_translation_initiation_fac- | 1.0 | 1.3 | 1.1 | −1.2 |
| tor_4E_4E_binding_protein_1 | | | | |
| W59510_elastin | −1.2 | −1.4 | −1.3 | −1.7 |
| AA869219_intelectin | −1.0 | −1.3 | −1.1 | 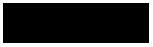 |
| AA822098_apolipoprotein_A-I | −1.9 | −1.5 | 1.1 | −2.4 |
| AI326604_cyclin_F | −1.1 | 1.0 | 1.2 | 1.0 |
| AA671135_myosin_heavy_chain,_cardiac_muscle,_adult | −1.1 | −1.3 | 1.1 | −1.2 |
| AA930278_ESTs | 1.1 | −1.3 | 1.1 | −1.2 |
| AI156185_cystic_fibrosis_transmembrane_con- | 1.2 | −1.42323+ | −1.16278+ | −1.4 |
| ductance_regulator_homolog | | | | |
| AA792001_RIKEN_cDNA_4931400A14_gene | −1.3 | 1.2 | 1.1 | −1.1 |
| AA672765_Niemann_Pick_type_C1 | 1.1 | −1.3 | 1.3 | −1.5 |
| AA792268_solute_carrier_family_4_(anion_exchanger),_member_1 | 1.0 | −1.5 | 1.1 | −1.1 |
| W53917_serine_hydromethyl_transferase_1_(soluble) | 1.0 | −1.1 | 1.3 | 1.1 |
| AI464601_ESTs | −1.06043+ | −1.40465+ | −1.02444+ | 1.0257+ |
| AA096815_Public_domain_EST | 1.2 | −1.3 | 1.2 | 1.5 |
| W80245_ESTs | −1.2 | 1.4 | −1.1 | −1.4 |
| AI551578_RIKEN_cDNA_1600013P15_gene | −1.3 | −1.4 | 1.3 | 1.0 |
| AA880295_immunoglobulin_joining_chain | 1.2 | −1.5 | −1.4 | 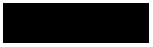 |
| AA871838_phospholipase_A2,_group_IIA_(platelets,_synovial_fluid) | −1.1 | −1.0 | −1.2 | 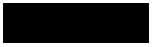 |
| AI529474_carbohydrate_(N-acetylglucosamine_6- | 1.0 | 1.4 | −1.1 | 1.3 |
| O)_sulfotransferase_5 | | | | |
| AA277642_ESTs,_Weakly_similar_to_A53399_L6_anti- | −1.2 | −1.1 | 1.4 | −1.1 |
| gen_-_mouse_[M.musculus] | | | | |
| AA667906_custeine-rich_protein_2 | 1.2 | 1.1 | 1.5 | −1.6 |
| AA44443_immunoglobulin_heavy_chain_6_(heavy_chain_IgM) | −1.2 | −1.8 | −1.4 | −1.1 |
| AA065522_RIKEN_cDNA_1700029G01_gene | −1.2 | −1.4 | −1.0 | −1.1 |
| AA656407_troponin_T1,_skeletal,_slow | 1.1 | −1.2 | 1.3 | −1.1 |
| W18463_thioether_S-methyltransferase | 1.0 | −1.5 | 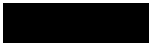 | −1.3 |
| AA065551_Mus_mus- | −1.0 | −1.2 | 1.2 | 1.1 |
| culus,_clone_IMAGE:3489758,_mRNA,_partial_cds | | | | |
| AA571365_Mus_musculus_MRPS6_mRNA_for_mito- | −1.2 | −1.1 | 1.3 | 1.3 |
| chondrial_ribosomal_protein_S6,_partial_cds | | | | |
| AA146546_ESTs | −1.2 | −1.4 | −1.4 | −1.5 |
| AA028420_small_proline-rich_protein_1A | 1.3 | −1.7 | −1.4 | −1.5 |
| AA718431_RIKEN_cDNA_1700020B19_gene | 1.0 | −1.2 | 1.2 | 1.0 |
| AA109757_solute_carrier_family_22_(organic_cation_transporter)- | −1.2 | −1.0 | −1.1 | −1.3 |
| like_2 | | | | |
| AI605613_RIKEN_cDNA_2210401F01_gene | 1.1 | −1.2 | 1.1 | −1.2 |
| AA671284_troponin_T2,_cardiac | 1.0 | −1.7 | −1.3 | −1.1 |
| AI644912_ESTs,_Weakly_similar_to_A58583_testosterone- | −1.2 | −1.2 | 1.1 | −1.1 |
| resistant_immunity- | | | | |
| associated_protein_IAP38_-_mouse_[M.musculus] | | | | |
| AI181090_cytochrome_450,_subfamily_IV,_B,_polypeptide_1 | 1.0 | −1.4 | −1.1 | −1.1 |
| AA764455_Mus_musculus,_clone_MGC:8241,_mRNA,_com- | 1.3 | −1.4 | −1.3 | −1.5 |
| plete_cds | | | | |
| AA117605_cytotoxic,_granule-associated_RNA-binding_protein_1 | 1.3 | −1.1 | 1.1 | −1.0 |
| AA544749_myosin_Ic | −1.1 | −1.1 | 1.0 | −1.2 |
| AA754696_immuno- | 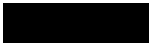 | −1.5 | 1.0 | −1.2 |
| globulin_heavy_chain_6_(heavy_chain_of_IgM) | | | | |
| W53837_mitogen_activated_protein_kinase_13 | 1.0 | 1.1 | −1.0 | −1.2 |
| AA600430_myeloblastosis_oncogene-like_2 | −1.1 | −1.2 | 1.1 | −1.1 |
| AA982280_N-myc_(and_STAT)_interactor | −1.2 | −1.2 | 1.5 | 1.0 |
| AA798953_transformation_related_protein_63 | 1.0 | −1.5 | −1.3 | 1.1 |
| AI046576_testis_specific_gene_1 | 1.4 | −1.3 | −1.4 | 1.5 |
| AA881032_Mus_musculus_ETL1_mRNA,_complete_cds | −1.2 | −1.0 | 1.3 | 1.1 |
| AA771566_troponin_C,_fast_skeletal | 1.1 | −1.6 | 1.4 | −1.1 |
| AA545451_gamma-glutamyl_transpeptidase | 1.0 | 1.4 | −1.8 | 1.4 |
| AA458072_adipocyte_complement_related_protein_of_30_kDa | 1.3 | −1.2 | −1.0 | −1.3 |
| AI047912_Mus_musculus_DNA_seg- | 1.07563+ | −1.57659+ | −1.09024+ | −1.13529+ |
| ment,_Chr_1,_Pasteur_institute_1_(D1Past1),_mRNA | | | | |
| AI385784_pleckstrin_2 | −1.1 | −1.1 | −1.0 | −1.2 |
| AA671573_chemokine_(C—C)_receptor_5 | 1.0 | −1.1 | 1.1 | −1.5 |
| W16358_mucin_1,_transmembrane | 1.5 | 1.4 | −1.2 | −2.2 |
| AI227406_lysosomal_trafficking_regulator | −1.2 | −1.25943+ | −1.02071+ | −1.01518+ |
| AA711611_RIKEN_cDNA_1110015E18_gene | 1.0 | −1.2 | 1.4 | 1.3 |
| AA760185_carbonic_anhydrase_6 | −1.2 | −1.7 | −1.0 | −1.2 |
| AI552705_NADPH_oxidase_4 | −1.0 | −1.0 | 1.3 | −1.0 |
| AA967824_arginine_vasopressin | 1.4 | −1.6 | −1.2 | −1.1 |
| AI098411_Public_domain_EST | −1.3 | −1.0 | 1.3 | −1.2 |
| AI594906_ESTs | −1.28447+ | −1.28228+ | 1.1 | 1.4 |
| AA414490_RIKEN_cDNA_1200014D15_gene | 1.3 | −1.4 | 1.4 | −1.2 |
| W13670_amino-terminal_enhancer_of_split | 1.1 | −1.0 | −1.0 | 1.0 |
| AA771229_ESTs,_Weakly_similar_to_zinc_finger_pro- | −2.4 | 1.2 | 1.3 | 1.2 |
| tein_95_[M.musculus] | | | | |
| AA760220_RIKEN_cDNA_2300002G24_gene | 1.1 | −1.0 | 1.2 | −1.1 |
| AI385961_cAMP_responsive_element_modulator | −1.1 | −1.1 | −1.0 | 1.1 |
| AI152670_ESTs | 1.16788+ | −1.54754+ | −1.26954+ | −1.39621+ |
| AA739040_lipoprotein_lipase | 1.1 | −1.4 | 1.1 | −1.3 |
| AA004134_centromere_autoantigen_A | 1.0 | −1.1 | 1.2 | 1.1 |
| AA105999_B-cell_src-homology_tyrosine_kinase | −1.4 | 1.089091+ | 1.5 | 1.2 |
| AA874245_RIKEN_cDNA_1810047J07_gene | 1.2 | −1.3 | −1.1 | −1.2 |
| AA756749_submaxillary_gland_androgen_regulated_protein_1 | −1.1 | −1.6 | 1.0 | −1.3 |
| AI894035_proprotein_convertase_subtilisin/kexin_type_5 | 1.1 | −1.49699+ | −1.0 | −1.2 |
| AI450453_ESTs,_Weakly_similar_to_T33424_hypothetical_pro- | 1.1 | 1.1 | −1.3 | 1.1 |
| tein_M01G5.4_-_Caenorhabditis_elegans_[C.elegans] | | | | |
| AA840053_actinin_alpha_2_assocated_LIM_protein | 1.1 | −1.3 | 1.0 | −1.3 |
| AA497592_ets_variant_gene_6_(TEL_oncogene) | −1.3 | 1.0 | 1.1 | −1.1 |
| AA667301_troponin_I,_cardiac | 1.4 | −1.6 | −1.1 | −2.1 |
| AA517353_nuclear_factor_of_kappa_light_chain_gene_en- | −1.0 | 1.2 | 1.1 | −1.1 |
| hancer_in_B-cells_inhibitor,_alpha | | | | |
| AI120822_serine_(or_cysteine)_proteinase_in- | 1.1 | −1.4 | −1.1 | −1.5 |
| hibitor,_clade_G_(C1_inhibitor),_member_1 | | | | |
| AA066016_RIKEN_cDNA_1190002H23_gene | −1.6 | 1.2 | 1.2 | 1.5 |
| AA061920_phospholipase_A2,_group_IIC | −1.3 | 1.0515+ | 1.3 | 1.1 |
| AA821953_paraoxonase_1 | 1.0 | −1.6 | 1.5 | −1.0 |
| AA416441_gene_rich_cluster,_C8_gene | 1.1 | −1.3 | 1.3 | 1.2 |
| W56995_hemoglobin_X,_alpha- | 1.1 | −1.6 | −1.3 | −1.2 |
| like_embryonic_chain_in_Hba_complex | | | | |
| AA771396_4-hydroxyphenylpyruvic_acid_dioxygenase | 1.1 | −1.4 | 1.2 | 1.1 |
| AA760223_epithelial_membrane_protein_3 | −1.1 | −1.1 | 1.4 | −1.0 |
| W55649_vasodilator-stimulated_phosphoprotein | 1.0 | −1.1 | 1.1 | −1.3 |
| AI019668_prostaglandin_I_receptor_(IP) | 1.01606+ | −1.10471+ | 1.08277+ | −1.3 |
| AA674177_cytochrome_P450,_2a4 | 1.2 | −1.6 | 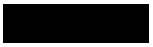 | 1.1 |
| AA437572_inner_centromere_protein | 1.1 | −1.4 | 1.1 | −1.3 |
| AI020539_secretory_leukocyte_protease_inhibitor | 1.1 | −1.5 | 1.1 | −1.1 |
| W83739_DNA_seg- | −1.2 | −1.0 | −1.5 | −1.5 |
| ment,_Chr_2,_Brigham_&_Women's_Genetics_1423_expressed | | | | |
| AA198236_kit_ligand | 1.0 | 1.10758+ | 1.00332+ | −1.26916+ |
| AI451780_prostate_specific_ets_transcription_factor | −1.1 | 1.4 | 1.1 | −1.5 |
| W11251_Fc_receptor,_IgE,_high_affinity_I,_gamma_polypeptide | 1.2 | 1.3 | 1.3 | 1.1 |
| AA691355_Mus_musculus_DNA_cytosine_methyl- | −1.4 | −1.3 | 1.2 | 1.0 |
| transferase_mRNA | | | | |
| AI324400_transforming_growth_factor,_beta_receptor_II | −1.1 | −1.1 | 1.5 | 1.0 |
| AA733479_syndecan_1 | 1.1 | 1.1 | 1.1 | −1.2 |
| AA617316_TEA_damain_family_member_2 | −1.3 | 1.1 | −1.1 | −1.1 |
| AA049060_inter-trypsin_inhibitor,_heavy_chain_4 | 1.5 | 1.3 | 1.2 | −1.2 |
| AA759995_procollagen,_type_XVII,_alpha_1 | 1.1 | −1.2 | 1.5 | −1.2 |
| AA028578_keratin_complex_2,_basic,_gene_4 | 1.2 | −1.9 | −1.3 | −1.0 |
| AA755961_insulin-like_growth_factor_2_receptor | −1.1 | −1.1 | 1.2 | −1.1 |
| AA727051_myeloid_leukemia_factor_1 | 1.1 | −1.2 | 1.4 | 1.1 |
| AA288467_glycosylation_dependent_cell_adhesion_molecule_1 | −1.0 | −1.8 | 1.2 | 1.2 |
| AA444639_sialyltransferase_7_((alpha-N-acetylneuraminyl_2,3-beta- | 1.4 | −1.2 | −1.0 | 1.0 |
| galactosyl-1,3)N-acetyl_galactosamide_alpha-2,6-sialyltransferase)_B | | | | |
| AA545691_thymidine_kinase_1 | 1.2 | −1.2 | 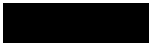 | −1.2 |
| AA106072_Public_domain_EST | 1.0 | −1.7 | 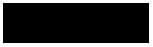 | −1.1 |
| AA414110_ESTs | −1.2 | −1.5 | −1.3 | −1.2 |
| AA414112_phosphatidylinositol_glycan,_class_A | 1.1 | −1.6 | 1.1 | −1.1 |
| AI046562_lactate_dehydrogenase_3,_C_chain,_sperm_specific | 1.2 | −1.6 | −1.3 | 1.5 |
| AI120958_RIKEN_cDNA_2700018N07_gene | −1.2 | 1.2 | 1.2 | −1.4 |
| W16012_uteroglobin | 1.3 | −1.6 | 1.2 | −1.4 |
| AA152940_immunoglobulin_kappa_chain_variable_28_(V28) | 1.3 | −1.6 | −1.5 | −1.0 |
| AI326527_preproacrosin | −1.3 | −1.2 | −1.0 | −1.2 |
| AA267626_mitogen_activated_pro- | 1.0 | 1.19439+ | 1.08191+ | 1.10045+ |
| tein_kinase_kinase_kinase_kinase_2 | | | | |
| AA607483_proteoglycan,_secretory_granule | 1.2 | −1.4 | 1.1 | 1.3 |
| AA982227_glutathione_S-transferase,_alpha_3 | 1.3 | −1.0 | 1.4 | −1.3 |
| AA220024_trophoblast_specific_protein | 1.5 | −2.0 | −1.2 | 1.5 |
| AA106793_plasminogen | 1.1 | −1.6 | 1.3 | 1.0 |
| W08947_non- | −1.8 | 1.1 | 1.3 | 1.0 |
| catalytic_region_of_tyrosine_kinase_adaptor_protein_2 | | | | |
| AI050373_RIKEN_cDNA_4921524P20_gene | −1.3 | 1.2 | −1.3 | −1.40942* |
| AA612413_chitinase,_acidic | −1.1 | −1.3 | 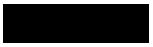 | −1.2 |
| AI327450_phospholipase_A2,_group_IB,_pancreas | 1.3 | 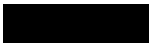 | 1.1 | −1.6 |
| AA117554_growth_arrest_specific_2 | 1.1 | −1.4 | 1.4 | 1.1 |
| AA529824_fatty_acid_binding_protein_2,_intestinal | −1.5 | −1.5 | −1.7 | 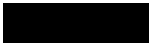 |
| W36635_carbonyl_reductase_2 | 1.2 | −1.9 | −1.4 | 1.4 |
| AI154217_poliovirus_sensitivity | −1.2 | 1.0 | 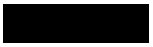 | −1.1 |
| AA237607_pyruvate_kinase_liver_and_blood_cell | −1.4 | −1.2 | 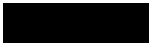 | −1.2 |
| AA600553_claudin_7 | −1.1 | 1.1 | −1.2 | −1.1 |
| AI019575_secreted_frizzled-related_sequence_protein_4 | 1.2 | −1.3 | −1.4 | −1.2 |
| W18281_RIKEN_cDNA_2310012I15_gene | 1.1 | 1.1 | −1.1 | 1.1 |
| AA028346_keratin_complex_1,_acidic,_gene_19 | −1.4 | −1.6 | −1.9 | −1.5 |
| AA061047_secreted_frizzled-related_sequence_protein_2 | 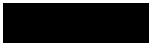 | −1.2 | −1.3 | 1.1 |
| AA220582_cytochrome_P450,_2f2 | 1.4 | −1.6 | 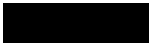 | −1.0 |
| AA108495_deoxyribonuclease_I | 1.1 | −1.8 | −1.1 | 1.2 |
| AA655921_neutral-salient_serine_arginine-rich | −1.1 | −1.4 | 1.1 | −1.0 |
| AI159406_casein_gamma | 1.1 | −1.7 | −1.5 | 1.2 |
| AI326575_glucose-6-phosphatase,_catalytic | 1.3 | −1.6 | 1.4 | 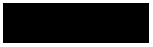 |
| AI182792_aquaporin_5 | 1.3 | −1.1 | −1.0 | −1.6 |
| AA062457_RIKEN_cDNA_061006F02_gene | 1.2 | −1.1 | 1.3 | 1.1 |
| AA286306_ESTs | −1.18303+ | −1.52374+ | −1.22332+ | −1.2 |
| AA549040_histone_acetyltransferase | −1.1 | −1.2 | −1.3 | −1.8 |
| AA656712_myosin,_heavy_polypeptide_1,_skeletal_muscle,_adult | 1.0 | −1.6 | 1.1 | 1.2 |
| AI256888_deleted_azoospermia-like | 1.1 | −1.2 | −1.1 | 1.0 |
| AA106125_cytochrome_450,_2a4 | 1.3 | −1.3 | 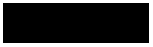 | 1.3 |
| AA064494_Public_domain_EST | 1.0 | −1.5 | −1.1 | 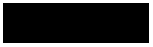 |
| AA066835_corticosteroid_binding_globulin | 1.0 | 1.2 | 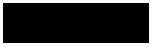 | −1.1 |
| AA106167_Public_domain_EST | −1.2 | −1.4 | −1.2 | −1.1 |
| AI876995_immunoglobulin-associated_alpha | −1.1 | −1.8 | −1.2 | −1.6 |
| AA681602_enolase_3,_beta_muscle | 1.1 | −1.7 | −1.0 | −1.4 |
| W17866_uterine-specific_proline-rich_acidic_protein | −1.4 | −1.7 | −1.2 | −1.4 |
| AI060394_ESTs | −1.6 | 1.3 | 1.4 | 1.3 |
| AA864136_lectin,_galactose_binding,_soluble_7 | 1.1 | −1.2 | 1.3 | −1.0 |
| AI552711_creatine_kinase,_muscle | 1.1 | −1.2 | 1.1 | −1.2 |
| AA980349_glutathione_peroxidase_2,_pseudogene_1 | −1.3 | −1.0 | 1.2 | −1.9 |
| AA756171_cysteine-rich_protein_3 | 1.1 | −1.7 | 1.0 | 1.1 |
| AA822346_odorant-binding_protein_Ia | −1.2 | −1.8 | −1.2 | −1.0 |
| AA276747_ESTs | 1.14674+ | 1.07209+ | 1.11618+ | 1.0 |
| AI390909_gap_junction_membrane_channel_protein_alpha_4 | −1.1 | 1.1 | 1.1 | −1.1 |
| AW496194_immuno- | 1.4 | −1.2 | −1.4 | −1.1 |
| globulin_kappa_chain_variable_20_(V20_family) | | | | |
| W82497_ubiquitin-like_1 | 1.3 | −1.8 | −1.0 | 1.1 |
| AI425461_ESTs,_Moderately_simi- | 1.1 | 1.1 | 1.2 | 1.1 |
| lar_to_1615347A_ras_p21_GTPase_activating_pro- | | | | |
| tein_[M.musculus] | | | | |
| AA189897_ESTs | 1.1 | 1.03128+ | 1.03168+ | 1.0 |
| AI664286_tumor_nucrosis_factor_receptor_superfamily,_member_9 | 1.3 | −2.3 | −1.1 | −1.3 |
| AA823639_RIKEN_cDNA_A930018B01_gene | 1.1 | −1.0 | 1.4 | −1.1 |
| AI594276_transcription_factor_AP-2,_alpha | −1.5 | −1.1 | −1.1 | −1.4 |
| AA122791_histocompatibility_2,_Q_region_locus_7 | 1.3 | −1.4 | 1.3 | −3.0 |
| AA759679_Ia-associated_invariant_chain | −1.0 | −1.8 | −1.6 | −1.9 |
| AA681596_procollagen-lysine,_2-oxoglutarate_5-dioxygenase_1 | 1.2 | −1.2 | −1.0 | −1.1 |
| AA538511_histocompatibility_2,_2,_D_region_locus_1 | 1.4 | −1.2 | 1.2 | −1.5 |
| AA273374_RIKEN_cDNA_1810008K20_gene | −1.0 | −1.0 | −1.3 | 1.4 |
| AA413953_ESTs,_Weakly_similar_to_KIAA0661_pro- | −1.2 | −1.1 | 1.1 | −1.4 |
| tein_[H.sapiens] | | | | |
| W11170_small_inducible_cytokine_A21a_(leucine) | −1.2 | −1.0 | 1.1 | 1.0 |
| AA717019_ATPase,_Ca++_trans- | −1.2 | −2.1 | −1.1 | −1.5 |
| porting,_cardiac_muscle,_fast_twich_1 | | | | |
| AA016759_mini_chromosome_maintenance_defi- | −1.4 | 1.1 | 1.1 | 1.1 |
| cient_6_(S._cercisiae) | | | | |
| W54403_ATPase,_Cu++_transporting,_beta_polypeptide | 1.2 | −2.1 | 1.1 | −1.0 |
| AA145479_ESTs | −1.2 | −1.3 | −1.3 | −1.2 |
| AI903969_sarcoglycan,_beta_(43kD_dystrophin- | 1.035+ | −1.0 | −1.2 | −1.4 |
| associated_glycoprotein) | | | | |
| AI226235_gap_junction_membrane_channel_protein_beta_4 | 1.1 | −1.3 | 1.1 | −1.0 |
| AA795319_myosin_light_chain,_phos- | −1.1 | −1.9 | −1.2 | −1.4 |
| phorylatable,_cardiac_ventricles | | | | |
| AI639807_ESTs,_Weakly_similar_to_E6- | 1.3 | −1.14976+ | 1.09666+ | −1.53665+ |
| AP_ubiquitin_protein_logase_[M.musculus] | | | | |
| AA212144_forkhead_box_A2 | −1.2 | 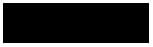 | 1.2 | −1.2 |
| AA871265_Public_domain_EST | 1.3 | −1.4 | −1.0 | 1.4 |
| AA270904_nuclear_receptor_subfamily_1,_group_H,_member_4 | 1.23141+ | 1.21202+ | 1.5 | 1.0 |
| AA738672_Mus_musculus_mRNA_for_thymopsin,_complete_cds | −1.1 | −1.2 | 1.2 | 1.1 |
| W36421_Moloney_leukemia_virus_10 | −1.1 | 1.0 | 1.1 | 1.2 |
| AI550101_ESTs | −1.13885+ | −1.00093+ | −1.00932+ | −1.0 |
| AA008383_phosphodiesterase_6D,_cGMP-specific,_rod,_delta | 1.2 | −1.03596+ | −1.5 | 1.0 |
| AI509041_Mus_musculus_Rep- 8,_complete_cds | −1.0 | 1.1 | 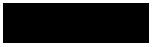 | −1.0 |
| AA549705_ESTs | 1.1 | 1.2204+ | 1.2 | 1.2 |
| W18330_tropomysin_2,_beta | 1.2 | −1.3 | 1.0 | −2.0 |
| AA815777_RIKEN_cDNA_1110007F23_gene | 1.0 | −1.1 | 1.2 | −1.3 |
| Q15812_desmin | −1.0 | −1.4 | −1.3 | −1.3 |
| AI595315_ESTs | 1.0 | −1.12188+ | −1.06667+ | −1.4 |
| AA549305_RIKEN_cDNA_4833427P12_gene | −1.11812+ | 1.22912+ | −1.03061+ | −1.19208+ |
| AA114721_RIKEN_cDNA_5830475I06_gene | −1.5 | 1.3 | −1.4 | −1.27884* |
| AA089188_uromodulin | −1.2 | −2.0 | −1.0 | 1.1 |
| AA839138_CD8_antigen,_alpha_chain | −1.3 | −1.08773+ | −1.3 | −1.8 |
| AA763276_small_muscle_protein,_X-linked | −1.7 | −1.4 | 1.1 | 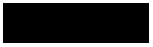 |
| AA474047_ESTs | −1.2 | −1.6 | −1.1 | −1.1 |

BRAIN

| 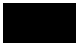 | int | col | 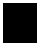 | lung |
|---|---|---|---|---|
| Description | PL-3B_BDEnor | PL-5B_BDEnor | PL-1B_BDEnor | PL-7B_BDEnor |
| 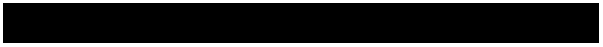 | 1.1 | −4.2 | 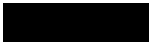 | −1.1 |
| 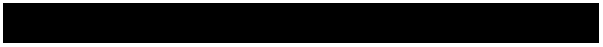 | 1.3 | −3.7 | 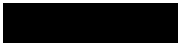 | −1.2 |
| 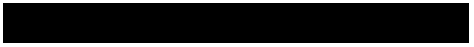 | −1.2 | −3.9332˜ | 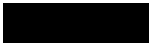 | −1.2 |
| 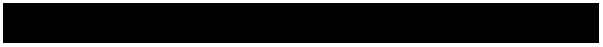 | −1.1 | −4.3 | 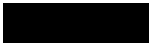 | 1.1 |
| 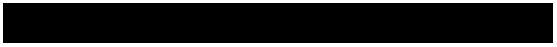 | 1.5 | 1.2409+˜ | 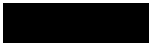 | 1.4 |
| 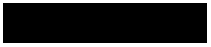 | 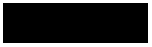 | −1.08065+˜ | 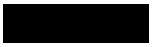 | 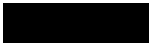 |
| 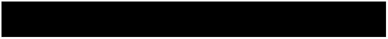 | 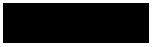 | 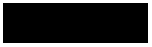 | 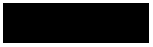 | 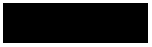 |
| 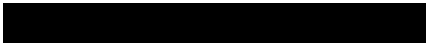 | 1.1 | −1.16438+˜ | 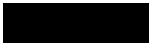 | 1.01996+ |
| AA821884_amylase_2,_pancreatic | −32.0 | −4.0 | 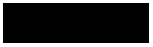 | −1.2 |
| AA588976_ESTs | 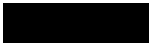 | 1.17361+˜ | 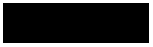 | 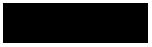 |
| W57281_Mest-linked_imprinted_transcript_1 | 1.1 | −1.43302+˜ | 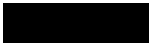 | 1.5 |
| AI549687_RIKEN_cDNA_5730406I15_gene | −1.0 | 1.01531+˜ | 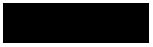 | 1.0 |
| AA002994_ESTs,_Moderately_similar_to_No_simi- larities_to_any_reported_proteins_[H.sapiens] | −1.1 | −1.28021+˜ | 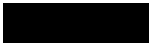 | 1.0 |
| AI529513_ESTs,_Moderately_similar_to_F26L_MOUSE_6PF- 2-K/FRU-2,6-P2ASE_LIVER_ISOZYME_[M.musculus] | 1.1 | −2.6 | 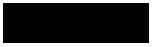 | 1.3 |
| AI605734_septin_3 | −1.7 | 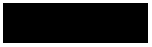 | 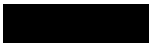 | 1.2 |
| AI464603_RIKEN_cDNA_0710001E13_gene | 1.4 | −1.08185+˜ | 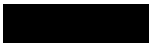 | 1.08953+ |
| AA717025_Mus_musculus_10_day_old_male_pan- crease_cDNA_RIKEN_full-length_enriched_library,_clone: 1810009A17,_full_insert_sequence | −25.5 | −5.2 | 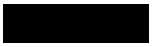 | −1.6 |
| AA117355_RIKEN_cDNA_1190006L01_gene | 1.0 | 1.23278+˜ | 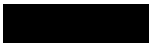 | 1.2 |
| AA267673_ESTs | 1.2 | −1.28358+˜ | 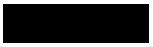 | 1.15552+ |
| AI594945_RIKEN_cDNA_6330406P08_gene | 1.09763+ | −1.02044+˜ | 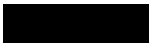 | −1.01366+ |
| AA119984_liver-specific_bHLH-Zip_transcription_factor | 1.5 | 1.31112˜ | 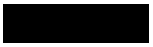 | 1.1 |
| AA522311_RIKEN_cDNA_0610038P07_gene | 1.2 | −1.08749+˜ | 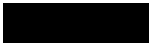 | 1.3 |
| AA967857_brain_protein | −1.5 | 1.00533˜ | 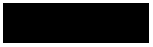 | 1.1 |
| AA253844_ESTs | 1.1 | −1.54029˜ | 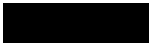 | 1.3 |
| AI122516_microtubule-associated_protein_6 | 1.4 | 1.28645+˜ | 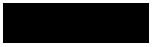 | 1.1 |
| AI430438_RIKEN_cDNA_3110023G01_gene | 1.5 | 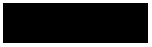 | 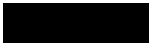 | 1.2 |
| AI550596_stearoyl-Coenzyme_A_desaturase_2 | 1.4 | −1.5 | 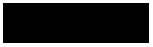 | 1.5 |
| AA200457_ESTs | 1.19151* | 1.03839+˜ | 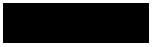 | −1.2 |
| AI324398_glycosylphosphatidylinositol_specific_phospholipase_D1 | 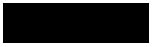 | −1.5 | 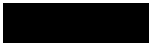 | −1.2 |
| AA929696_peptidyl_arginine_deiminase,_type_II | 1.1 | −1.10637+˜ | 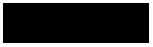 | −1.2 |
| AA123314_ESTs,_Highly_similar_to_S68176_TOG_pro- tein_[H.sapiens] | 1.1 | −1.0657˜ | 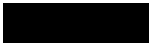 | 1.3 |
| AA473684_RIKEN_cDNA_C030002O17_gene | 1.37779+ | −1.21258+˜ | 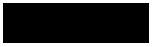 | 1.50959+ |
| AA518686_ESTs | 1.5 | −1.5 | 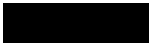 | 1.4 |
| AA412921_RIKEN_cDNA_4921514I20_gene | 1.4 | −1.23027˜ | 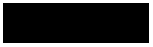 | 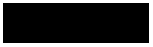 |
| AA051041_potassium_voltage- gated_channel,_subfamily_Q,_member_2 | 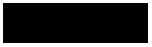 | 1.50089+˜ | 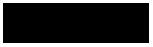 | −1.0726+ |
| AI604587_ESTs | 1.5 | −1.02494+˜ | 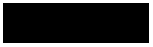 | 1.5 |
| AA059783_Public_domain_EST | 1.05655+ | −1.18681+˜ | 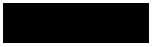 | 1.18474+ |
| AA466153_ESTs | −2.4 | −1.16147+˜ | 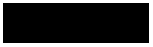 | 1.03478+ |
| AA222952_zuotin_related_factor_2 | −1.48418+ | −1.47865+˜ | 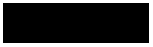 | 1.03133+ |
| AI552958_RIKEN_cDNA_1110003E08_gene | 1.4 | −1.16793˜ | 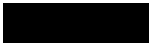 | 1.1 |
| AA416143_ESTs | 1.2 | −1.04633+˜ | 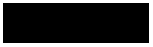 | 1.00503+ |
| AI558253_RIKEN_cDNA_1500015J03_gene | 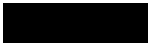 | 1.32632+˜ | 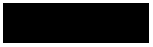 | 1.37534+ |
| AI553232_ribosomal_protein_S6 | 1.2 | −1.20582+˜ | 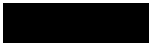 | 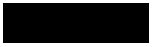 |
| AA511234_ESTs | −1.6 | −1.6533+˜ | 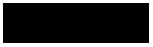 | 1.0 |
| AI550872_RAB11a,_member_RAS_oncogene_family | 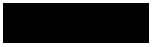 | −1.31004+˜ | 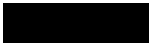 | 1.3 |
| AA839604_ESTs | 1.0 | −1.36963+˜ | 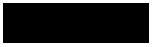 | 1.0 |
| AA120302_ESTs | 1.4 | −1.30774+˜ | 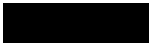 | 1.1 |
| AA412912_ESTs | 1.5 | −1.07075+˜ | 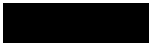 | 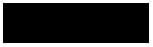 |
| AA003144_ESTs | 1.00927+ | 1.05326+˜ | 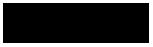 | −1.02885+ |
| AA387339_ESTs | −1.7 | −1.14346˜ | −1.9 | −1.1 |
| AA738833_solute_carrier_family_1,_member_7 | −1.2 | 1.0 | −1.9 | 1.2 |
| AI893895_cytochrome_P450,_2d9 | 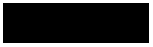 | 1.1 | −1.9 | −3.7 |
| W30045_eukaryotic_translation_initiation_fac- | 1.0 | −1.0 | −1.9 | −1.2 |
| tor_4E_4E_binding_protein_1 | | | | |
| W59510_elastin | −1.2 | 1.1 | −1.9 | −1.0 |
| AA869219_intelectin | 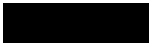 | 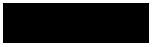 | −1.9 | 1.3 |
| AA822098_apolipoprotein_A-I | 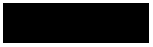 | 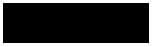 | −1.9 | −9.0 |
| AI326604_cyclin_F | −1.0 | 1.1 | −1.9 | −1.1 |
| AA671135_myosin_heavy_chain,_cardiac_muscle,_adult | 1.1 | 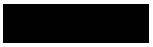 | −1.9 | −1.3 |
| AA930278_ESTs | −1.1 | 1.36967˜ | −1.9 | 1.1 |
| AI156185_cystic_fibrosis_transmembrane_con- | −1.9 | −1.16394˜ | −1.9 | 1.2 |
| ductance_regulator_homolog | | | | |
| AA792001_RIKEN_cDNA_4931400A14_gene | −1.2 | −1.06494+˜ | −1.9 | 1.1 |
| AA672765_Niemann_Pick_type_C1 | 1.0 | 1.27447˜ | −1.9 | −1.1 |
| AA792268_solute_carrier_family_4_(anion_exchanger),_member_1 | −1.1 | 1.2 | −1.9 | −1.5 |
| W53917_serine_hydromethyl_transferase_1_(soluble) | −1.1 | −1.6 | −1.9 | −1.2 |
| AI464601_ESTs | −1.14202+ | −1.123593+˜ | −1.9 | 1.2 |
| AA096815_Public_domain_EST | 1.2 | 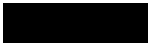 | −1.9 | −1.2 |
| W80245_ESTs | −1.3 | 1.3 | −1.9 | −1.5 |
| AI551578_RIKEN_cDNA_1600013P15_gene | −1.6 | 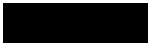 | −1.9 | −1.1 |
| AA880295_immunoglobulin_joining_chain | −2.2 | −2.4 | −1.9 | −1.1 |
| AA871838_phospholipase_A2,_group_IIA_(platelets,_synovial_fluid) | 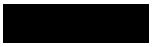 | 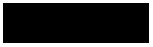 | −1.9 | −1.3 |
| AI529474_carbohydrate_(N-acetylglucosamine_6- | 1.2 | 1.07225+˜ | −1.9 | 1.2 |
| O)_sulfotransferase_5 | | | | |
| AA277642_ESTs,_Weakly_similar_to_A53399_L6_anti- gen_-_mouse_[M.musculus] | −1.3 | 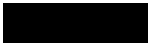 | −1.9 | −1.1 |
| AA667906_custeine-rich_protein_2 | −1.3 | −1.3 | −1.9 | −1.5 |
| AA44443_immunoglobulin_heavy_chain_6_(heavy_chain_IgM) | 1.2 | −2.1 | −1.9 | 1.1 |
| AA065522_RIKEN_cDNA_1700029G01_gene | −1.3 | 1.2 | −1.9 | −1.2 |
| AA656407_troponin_T1,_skeletal,_slow | 1.2 | 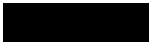 | −1.9 | −1.1 |
| W18463_thioether_S-methyltransferase | −1.6 | −1.6 | −1.9 | −1.0 |
| AA065551_Mus_mus- culus,_clone_IMAGE:3489758,_mRNA,_partial_cds | 1.1 | 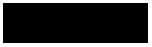 | −1.9 | −1.2 |
| AA571365_Mus_musculus_MRPS6_mRNA_for_mito- chondrial_ribosomal_protein_S6,_partial_cds | 1.3 | 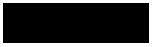 | −1.9 | −2.2 |
| AA146546_ESTs | −1.1 | −1.03444˜ | −1.9 | −1.1 |
| AA028420_small_proline-rich_protein_1A | −1.9 | −1.2 | −1.9 | −1.2 |
| AA718431_RIKEN_cDNA_1700020B19_gene | 1.2 | 1.46773+˜ | −1.9 | −1.2 |
| AA109757_solute_carrier_family_22_(organic_cation_transporter)- | −1.3 | 1.2 | −1.9 | −1.0 |
| like_2 | | | | |
| AI605613_RIKEN_cDNA_2210401F01_gene | −1.7 | 1.4 | −1.9 | 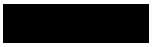 |
| AA671284_troponin_T2,_cardiac | −1.2 | −1.0 −1.9 | −1.6 | |
| AI644912_ESTs,_Weakly_similar_to_A58583_testosterone- resistant_immunity- associated_protein_IAP38_-_mouse_[M.musculus] | 1.3 | 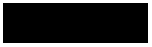 | −1.9 | −1.1 |
| AI181090_cytochrome_450,_subfamily_IV,_B,_polypeptide_1 | 1.1 | 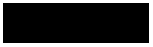 | −1.9 | −1.2 |
| AA764455_Mus_musculus,_clone_MGC:8241,_mRNA,_com- | −1.4 | −1.32023+˜ | −1.9 | 1.2 |
| plete_cds | | | | |
| AA117605_cytotoxic,_granule-associated_RNA-binding_protein_1 | 1.1 | −1.1 | −1.9 | 1.0 |
| AA544749_myosin_Ic | −1.4 | 1.0 | −1.9 | 1.1 |
| AA754696_immuno- | −1.9 | −3.2 | −1.9 | −1.0 |
| globulin_heavy_chain_6_(heavy_chain_of_IgM) | | | | |
| W53837_mitogen_activated_protein_kinase_13 | −1.0 | −1.5 | −1.9 | −1.1 |
| AA600430_myeloblastosis_oncogene-like_2 | −1.2 | 1.24314˜ | −2.0 | −1.1 |
| AA982280_N-myc_(and_STAT)_interactor | −1.2 | 1.0219˜ | −2.0 | −1.2 |
| AA798953_transformation_related_protein_63 | 1.2 | 1.35161+˜ | −2.0 | −1.1 |
| AI046576_testis_specific_gene_1 | 1.2 | −1.05001˜ | −2.0 | 1.3 |
| AA881032_Mus_musculus_ETL1_mRNA,_complete_cds | 1.1 | 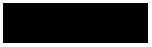 | −2.0 | −1.2 |
| AA771566_troponin_C,_fast_skeletal | 1.0 | 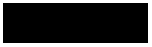 | −2.0 | −1.2 |
| AA545451_gamma-glutamyl_transpeptidase | 1.4 | 1.5 | −2.0 | −1.5 |
| AA458072_adipocyte_complement_related_protein_of_30_kDa | 1.0 | −2.4 | −2.0 | −1.1 |
| AI047912_Mus_musculus_DNA_seg- | −1.21199+ | −1.58604+˜ | −2.0 | 1.15955+ |
| ment,_Chr_1,_Pasteur_institute_1_(D1Past1),_mRNA | | | | |
| AI385784_pleckstrin_2 | 1.1 | −1.1 | −2.0 | 1.0 |
| AA671573_chemokine_(C—C)_receptor_5 | −1.1 | −1.22865˜ | −2.0 | −1.2 |
| W16358_mucin_1,_transmembrane | −1.3 | −1.1 | −2.0 | 1.0 |
| AI227406_lysosomal_trafficking_regulator | −1.22397+ | 1.08155+˜ | −2.0 | 1.5 |
| AA711611_RIKEN_cDNA_1110015E18_gene | 1.1 | 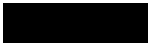 | −2.0 | −1.2 |
| AA760185_carbonic_anhydrase_6 | −1.2 | 1.5 | −2.0 | 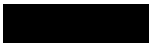 |
| AI552705_NADPH_oxidase_4 | 1.4 | 1.41103+˜ | −2.0 | 1.1 |
| AA967824_arginine_vasopressin | −1.9 | −2.7 | −2.0 | −1.4 |
| AI098411_Public_domain_EST | −1.1 | 1.0 | −2.0 | −1.2 |
| AI594906_ESTs | −1.1 | 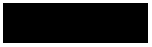 | −2.0 | −1.2 |
| AA414490_RIKEN_cDNA_1200014D15_gene | 1.0 | 1.07353˜ | −2.0 | −1.8 |
| W13670_amino-terminal_enhancer_of_split | −1.0 | −1.2 | −2.0 | −1.1 |
| AA771229_ESTs,_Weakly_similar_to_zinc_finger_pro- tein_95_[M.musculus] | 1.2 | 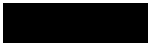 | −2.0 | −3.3 |
| AA760220_RIKEN_cDNA_2300002G24_gene | 1.3 | 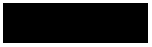 | −2.0 | −1.3 |
| AI385961_cAMP_responsive_element_modulator | 1.2 | 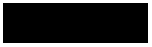 | −2.0 | −1.2 |
| AI152670_ESTs | −1.48435+ | −1.01746+˜ | −2.0 | −1.03099+ |
| AA739040_lipoprotein_lipase | −1.0 | 1.2 | −2.0 | 1.1 |
| AA004134_centromere_autoantigen_A | 1.1 | 1.1 | −2.0 | −1.6 |
| AA105999_B-cell_src-homology_tyrosine_kinase | 1.0 | 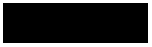 | −2.0 | −1.4 |
| AA874245_RIKEN_cDNA_1810047J07_gene | −1.1 | −1.36336+˜ | −2.0 | 1.2 |
| AA756749_submaxillary_gland_androgen_regulated_protein_1 | −1.2 | 1.5 | −2.0 | 1.3 |
| AI894035_proprotein_convertase_subtilisin/kexin_type_5 | 1.3 | 1.43771+˜ | −2.0 | −1.0 |
| AI450453_ESTs,_Weakly_similar_to_T33424_hypothetical_pro- | 1.2 | 1.19269+˜ | −2.0 | −1.0 |
| tein_M01G5.4_-_Caenorhabditis_elegans_[C.elegans] | | | | |
| AA840053_actinin_alpha_2_assocated_LIM_protein | 1.1 | 1.0 | −2.0 | −1.0 |
| AA497592_ets_variant_gene_6_(TEL_oncogene) | 1.2 | 1.1 | −2.0 | −1.4 |
| AA667301_troponin_I,_cardiac | 1.3 | 1.41297˜ | −2.0 | −2.0 |
| AA517353_nuclear_factor_of_kappa_light_chain_gene_en- | 1.1 | −1.2 | −2.0 | −1.3 |
| hancer_in_B-cells_inhibitor,_alpha | | | | |
| AI120822_serine_(or_cysteine)_proteinase_in- | 1.2 | −1.5 | −2.0 | −1.0 |
| hibitor,_clade_G_(C1_inhibitor),_member_1 | | | | |
| AA066016_RIKEN_cDNA_1190002H23_gene | 1.2 | 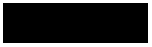 | −2.0 | −2.4 |
| AA061920_phospholipase_A2,_group_IIC | 1.1 | 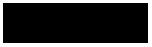 | −2.0 | −1.2 |
| AA821953_paraoxonase_1 | 1.3 | 1.30398˜ | −2.1 | −1.4 |
| AA416441_gene_rich_cluster,_C8_gene | 1.1 | 1.4 | −2.1 | −1.3 |
| W56995_hemoglobin_X,_alpha- | −2.2 | 1.11232˜ | −2.1 | −2.0 |
| like_embryonic_chain_in_Hba_complex | | | | |
| AA771396_4-hydroxyphenylpyruvic_acid_dioxygenase | 1.4 | 1.1 | −2.1 | −5.2 |
| AA760223_epithelial_membrane_protein_3 | 1.2 | 1.0434˜ | −2.1 | −1.3 |
| W55649_vasodilator-stimulated_phosphoprotein | 1.1 | −1.1 | −2.1 | −1.3 |
| AI019668_prostaglandin_I_receptor_(IP) | −1.31076+ | −1.49409+˜ | −2.1 | −1.11103+ |
| AA674177_cytochrome_P450,_2a4 | 1.3 | 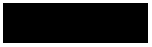 | −2.1 | 1.2 |
| AA437572_inner_centromere_protein | −1.2 | 1.23932˜ | −2.1 | −1.1 |
| AI020539_secretory_leukocyte_protease_inhibitor | 1.0 | 1.1 | −2.1 | 1.1 |
| W83739_DNA_seg- | 1.0 | −1.66255+˜ | −2.1 | −1.2 |
| ment,_Chr_2,_Brigham_&_Women's_Genetics_1423_expressed | | | | |
| AA198236_kit_ligand | −1.5351+ | −1.09631+˜ | −2.1 | 1.1 |
| AI451780_prostate_specific_ets_transcription_factor | −1.1 | −2.44752˜ | −2.1 | −1.0 |
| W11251_Fc_receptor,_IgE,_high_affinity_I,_gamma_polypeptide | 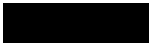 | −1.02621+˜ | −2.1 | 1.1 |
| AA691355_Mus_musculus_DNA_cytosine_methyl- transferase_mRNA | 1.0 | 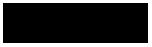 | −2.1 | −1.6 |
| AI324400_transforming_growth_factor,_beta_receptor_II | 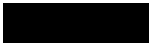 | 1.3 | −2.1 | 1.1 |
| AA733479_syndecan_1 | 1.1 | −1.4 | −2.1 | 1.1 |
| AA617316_TEA_damain_family_member_2 | −1.2 | 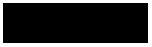 | −2.1 | −1.5 |
| AA049060_inter-trypsin_inhibitor,_heavy_chain_4 | −1.0 | 1.05302˜ | −2.1 | −2.2 |
| AA759995_procollagen,_type_XVII,_alpha_1 | 1.4 | 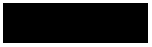 | −2.1 | −1.2 |
| AA028578_keratin_complex_2,_basic,_gene_4 | −1.0 | 1.44671˜ | −2.1 | 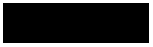 |
| AA755961_insulin-like_growth_factor_2_receptor | −1.1 | 1.4 | −2.1 | −1.1 |
| AA727051_myeloid_leukemia_factor_1 | 1.3 | 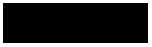 | −2.1 | −1.1 |
| AA288467_glycosylation_dependent_cell_adhesion_molecule_1 | −1.1 | 1.2 | −2.1 | −1.3 |
| AA444639_sialyltransferase_7_((alpha-N-acetylneuraminyl_2,3-beta- | −1.2 | −1.2 | −2.2 | −1.1 |
| galactosyl-1,3)N-acetyl_galactosamide_alpha-2,6-sialyltransferase)_B | | | | |
| AA545691_thymidine_kinase_1 | −1.1 | 1.24271˜ | −2.2 | −1.1 |
| AA106072_Public_domain_EST | 1.0 | 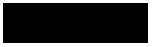 | −2.2 | −1.8 |
| AA414110_ESTs | 1.0 | 1.02832˜ | −2.2 | −1.0 |
| AA414112_phosphatidylinositol_glycan,_class_A | −1.6 | 1.15321+˜ | −2.2 | −1.0 |
| AI046562_lactate_dehydrogenase_3,_C_chain,_sperm_specific | 1.3 | 1.27823˜ | −2.2 | −1.0 |
| AI120958_RIKEN_cDNA_2700018N07_gene | −1.5 | 1.0 | −2.2 | −1.2 |
| W16012_uteroglobin | −1.6 | 1.0 | −2.2 | −1.3 |
| AA152940_immunoglobulin_kappa_chain_variable_28_(V28) | −2.1 | −3.1 | −2.2 | −1.3 |
| AI326527_preproacrosin | 1.0 | 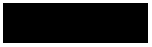 | −2.2 | −1.3 |
| AA267626_mitogen_activated_pro- | −1.00411+ | −1.21849+˜ | −2.2 | 1.2 |
| tein_kinase_kinase_kinase_kinase_2 | | | | |
| AA607483_proteoglycan,_secretory_granule | 1.2 | 1.1 | −2.2 | −1.2 |
| AA982227_glutathione_S-transferase,_alpha_3 | −1.0 | 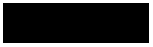 | −2.2 | −1.2 |
| AA220024_trophoblast_specific_protein | 1.1 | −1.0 | −2.2 | −1.2 |
| AA106793_plasminogen | 1.1 | 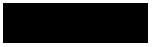 | −2.2 | −3.6 |
| W08947_non- | 1.2 | 1.4 | −2.2 | −2.2 |
| catalytic_region_of_tyrosine_kinase_adaptor_protein_2 | | | | |
| AI050373_RIKEN_cDNA_4921524P20_gene | 1.0 | −1.22069+˜ | −2.2 | −1.2 |
| AA612413_chitinase,_acidic | 1.2 | 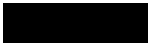 | −2.3 | 1.2 |
| AI327450_phospholipase_A2,_group_IB,_pancreas | −5.7 | 1.01624˜ | −2.3 | −1.6 |
| AA117554_growth_arrest_specific_2 | 1.3 | 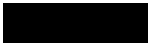 | −2.3 | −1.3 |
| AA529824_fatty_acid_binding_protein_2,_intestinal | −1.1 | 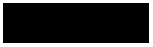 | −2.3 | 1.0 |
| W36635_carbonyl_reductase_2 | 1.0 | 1.3 | −2.3 | 1.1 |
| AI154217_poliovirus_sensitivity | 1.2 | 1.3 | −2.3 | −1.4 |
| AA237607_pyruvate_kinase_liver_and_blood_cell | 1.5 | 1.3 | −2.3 | −1.2 |
| AA600553_claudin_7 | 1.2 | −1.3 | −2.3 | −1.2 |
| AI019575_secreted_frizzled-related_sequence_protein_4 | −1.2 | 1.30118˜ | −2.3 | −1.3 |
| W18281_RIKEN_cDNA_2310012I15_gene | 1.1 | −1.1 | −2.3 | 1.1 |
| AA028346_keratin_complex_1,_acidic,_gene_19 | 1.0 | −1.2 | −2.3 | 1.1 |
| AA061047_secreted_frizzled-related_sequence_protein_2 | 1.1 | −1.08865˜ | −2.3 | 1.3 |
| AA220582_cytochrome_P450,_2f2 | 1.1 | 1.1 | −2.3 | 1.5 |
| AA108495_deoxyribonuclease_I | 1.4 | 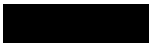 | −2.3 | −1.2 |
| AA655921_neutral-salient_serine_arginine-rich | −1.1 | 1.5 | −2.3 | −1.1 |
| AI159406_casein_gamma | 1.3 | 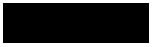 | −2.3 | −1.3 |
| AI326575_glucose-6-phosphatase,_catalytic | 1.4 | 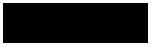 | −2.3 | −1.2 |
| AI182792_aquaporin_5 | −1.8 | 1.03979˜ | −2.3 | 1.1 |
| AA062457_RIKEN_cDNA_061006F02_gene | 1.1 | −1.4 | −2.3 | −1.9 |
| AA286306_ESTs | −1.3 | −1.22297+˜ | −2.4 | 1.1 |
| AA549040_histone_acetyltransferase | −1.1 | −1.10371+˜ | −2.4 | −1.2 |
| AA656712_myosin,_heavy_polypeptide_1,_skeletal_muscle,_adult | 1.2 | 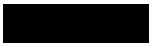 | −2.4 | −1.4 |
| AI256888_deleted_azoospermia-like | −1.1 | −1.06147+˜ | −2.4 | −1.2 |
| AA106125_cytochrome_450,_2a4 | 1.4 | 1.0 | −2.4 | 1.3 |
| AA064494_Public_domain_EST | 1.3 | 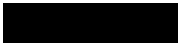 | −2.4 | 1.2 |
| AA066835_corticosteroid_binding_globulin | 1.2 | 1.4 | −2.4 | −2.3 |
| AA106167_Public_domain_EST | −1.1 | 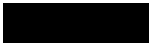 | −2.4 | −1.6 |
| AI876995_immunoglobulin-associated_alpha | −1.1 | −2.1 | −2.4 | −1.2 |
| AA681602_enolase_3,_beta_muscle | 1.3 | −1.5 | −2.5 | −1.3 |
| W17866_uterine-specific_proline-rich_acidic_protein | −1.2 | 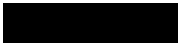 | −2.5 | −1.0 |
| AI060394_ESTs | 1.3 | 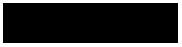 | −2.5 | −1.49757+ |
| AA864136_lectin,_galactose_binding,_soluble_7 | 1.2 | 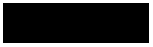 | −2.5 | −1.5 |
| AI552711_creatine_kinase,_muscle | 1.3 | 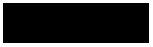 | −2.5 | −1.2 |
| AA980349_glutathione_peroxidase_2,_pseudogene_1 | 1.1 | −2.4 | −2.5 | −1.5 |
| AA756171_cysteine-rich_protein_3 | 1.3 | 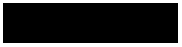 | −2.5 | −1.6 |
| AA822346_odorant-binding_protein_Ia | −1.2 | 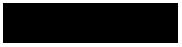 | −2.6 | −1.6 |
| AA276747_ESTs | −1.5 | −1.10511+˜ | −2.6 | 1.1 |
| AI390909_gap_junction_membrane_channel_protein_alpha_4 | 1.1 | −1.26782+˜ | −2.6 | 1.1 |
| AW496194_immuno- | −1.9 | −2.9 | −2.6 | −1.6 |
| globulin_kappa_chain_variable_20_(V20_family) | | | | |
| W82497_ubiquitin-like_1 | −1.0 | −1.1 | −2.7 | −1.5 |
| AI425461_ESTs,_Moderately_simi- | 1.2 | 1.17621+˜ | −2.7 | 1.1 |
| lar_to_1615347A_ras_p21_GTPase_activating_pro- | | | | |
| tein_[M.musculus] | | | | |
| AA189897_ESTs | −1.2 | −1.30212+˜ | −2.7 | 1.23177+ |
| AI664286_tumor_nucrosis_factor_receptor_superfamily,_member_9 | −1.3 | 1.2 | −2.7 | −1.3 |
| AA823639_RIKEN_cDNA_A930018B01_gene | 1.0 | 1.37539˜ | −2.7 | 1.0 |
| AI594276_transcription_factor_AP-2,_alpha | 1.2 | 1.30517˜ | −2.7 | −1.4 |
| AA122791_histocompatibility_2,_Q_region_locus_7 | 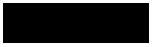 | −1.2 | −2.8 | −1.2 |
| AA759679_Ia-associated_invariant_chain | 1.5 | −3.7 | −2.8 | −1.0 |
| AA681596_procollagen-lysine,_2-oxoglutarate_5-dioxygenase_1 | −1.1 | −1.3 | −2.8 | −1.0 |
| AA538511_histocompatibility_2,_2,_D_region_locus_1 | 1.5 | −1.5 | −2.8 | 1.1 |
| AA273374_RIKEN_cDNA_1810008K20_gene | 1.1 | 1.06792+˜ | −2.8 | 1.0 |
| AA413953_ESTs,_Weakly_similar_to_KIAA0661_pro- | −1.2 | 1.05059˜ | −2.8 | −1.0 |
| tein_[H.sapiens] | | | | |
| W11170_small_inducible_cytokine_A21a_(leucine) | 1.3 | −2.7 | −2.8 | −1.1 |
| AA717019_ATPase,_Ca++_trans- | −1.3 | 1.2 | −2.8 | −1.4 |
| porting,_cardiac_muscle,_fast_twich_1 | | | | |
| AA016759_mini_chromosome_maintenance_defi- cient_6_(S._cercisiae) | 1.3 | 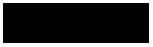 | −2.9 | −2.0 |
| W54403_ATPase,_Cu++_transporting,_beta_polypeptide | 1.2 | 1.3 | −2.9 | −7.9 |
| AA145479_ESTs | −1.0 | 1.16703+˜ | −2.9 | −1.2 |
| AI903969_sarcoglycan,_beta_(43kD_dystrophin- | 1.0 | −1.42644+˜ | −2.9 | 1.1 |
| associated_glycoprotein) | | | | |
| AI226235_gap_junction_membrane_channel_protein_beta_4 | 1.1 | 1.49281˜ | −3.0 | −1.6 |
| AA795319_myosin_light_chain,_phos- phorylatable,_cardiac_ventricles | −1.3 | 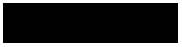 | −3.0 | −1.8 |
| AI639807_ESTs,_Weakly_similar_to_E6- | −1.1162+ | −1.6245+˜ | −3.0 | 1.11591+ |
| AP_ubiquitin_protein_logase_[M.musculus] | | | | |
| AA212144_forkhead_box_A2 | −1.7 | 1.16246+˜ | −3.1 | −1.3 |
| AA871265_Public_domain_EST | −1.7 | −3.0 | −3.1 | −1.3 |
| AA270904_nuclear_receptor_subfamily_1,_group_H,_member_4 | 1.20848+ | −1.53218+˜ | −3.1 | −1.09991+ |
| AA738672_Mus_musculus_mRNA_for_thymopsin,_complete_cds | 1.2 | 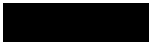 | −3.1 | −1.3 |
| W36421_Moloney_leukemia_virus_10 | 1.0 | −1.09155˜ | −3.3 | 1.1 |
| AI550101_ESTs | 1.06911+ | 1.21429+˜ | −3.4 | −1.15513+ |
| AA008383_phosphodiesterase_6D,_cGMP-specific,_rod,_delta | 1.1 | −1.1258+˜ | −3.4 | −1.0 |
| AI509041_Mus_musculus_Rep- | −1.3 | −1.17893+˜ | −3.4 | −1.1 |
| 8,_complete_cds | | | | |
| AA549705_ESTs | −1.0 | 1.0769+˜ | −3.5 | −1.3 |
| W18330_tropomysin_2,_beta | 1.2 | −1.1 | −3.5 | −1.5 |
| AA815777_RIKEN_cDNA_1110007F23_gene | 1.2 | 1.1 | −3.5 | 1.0 |
| Q15812_desmin | 1.1 | −1.8 | −3.6 | −1.7 |
| AI595315_ESTs | −1.21568+ | −1.29621+˜ | −3.7 | 1.03263+ |
| AA549305_RIKEN_cDNA_4833427P12_gene | −1.1371+ | 1.02682+˜ | −3.7 | −1.05244+ |
| AA114721_RIKEN_cDNA_5830475I06_gene | 1.3 | −1.8 | −3.7 | −1.2 |
| AA089188_uromodulin | −1.3 | 1.0 | −4.0 | −1.4 |
| AA839138_CD8_antigen,_alpha_chain | 1.2 | −2.63867+˜ | −4.0 | 1.0 |
| AA763276_small_muscle_protein,_X-linked | 1.2 | 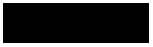 | −4.6 | −2.5 |
| AA474047_ESTs | −1.8 | 1.1 | −6.0 | 1.0 |

BRAIN

| 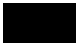 | blad | kidn | pl/ut | mam |
|---|---|---|---|---|
| Description | PL-9B_BDEnor | PL-10B_BDEnor | PL-11B_BDEnor | PL-12B_BDEnor |
| 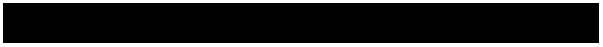 | −1.3 | 1.5 | −1.8 | −2.0 |
| 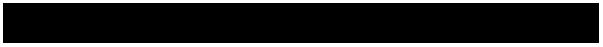 | −1.0 | 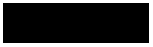 | −1.7 | −1.6 |
| 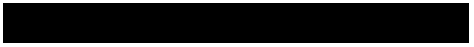 | −1.2 | 1.3 | −1.5 | −1.5 |
| 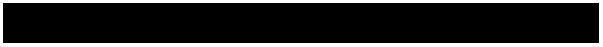 | −1.3 | 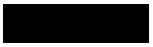 | −1.7 | −2.0 |
| 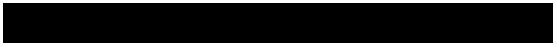 | 1.5 | 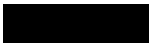 | 1.5 | 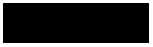 |
| 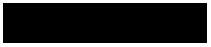 | 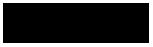 | 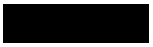 | 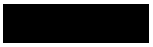 | 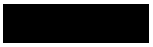 |
| 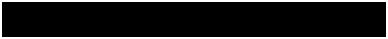 | 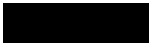 | 1.5 | 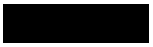 | 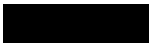 |
| 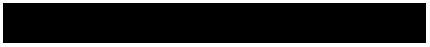 | −1.1 | −1.1 | 1.1 | 1.1 |
| AA821884_amylase_2,_pancreatic | −4.4 | −1.1 | −1.1 | −1.2 |
| AA588976_ESTs | 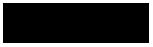 | 1.4 | 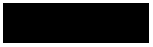 | 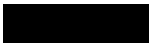 |
| W57281_Mest-linked_imprinted_transcript_1 | 1.4 | 1.1 | 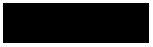 | 1.2 |
| AI549687_RIKEN_cDNA_5730406I15_gene | 1.1 | 1.3 | 1.1 | 1.1 |
| AA002994_ESTs,_Moderately_similar_to_No_simi- | −1.2 | 1.2 | −1.2 | −1.0 |
| larities_to_any_reported_proteins_[H.sapiens] | | | | |
| AI529513_ESTs,_Moderately_similar_to_F26L_MOUSE_6PF- | −1.1 | 1.4 | −1.3 | −1.3 |
| 2-K/FRU-2,6-P2ASE_LIVER_ISOZYME_[M.musculus] | | | | |
| AI605734_septin_3 | 1.1 | −1.2 | 1.3 | 1.1 |
| AI464603_RIKEN_cDNA_0710001E13_gene | 1.4 | 1.2 | 1.4 | 1.4 |
| AA717025_Mus_musculus_10_day_old_male_pan- | −2.9 | 1.2 | −1.1 | 1.4 |
| crease_cDNA_RIKEN_full-length_enriched_library,_clone: | | | | |
| 1810009A17,_full_insert_sequence | | | | |
| AA117355_RIKEN_cDNA_1190006L01_gene | 1.2 | −1.0 | 1.3 | 1.4 |
| AA267673_ESTs | 1.1 | 1.3 | 1.2 | 1.2 |
| AI594945_RIKEN_cDNA_6330406P08_gene | −1.02153+ | 1.4 −1.1 | 1.0976+ | |
| AA119984_liver-specific_bHLH-Zip_transcription_factor | 1.2 | 1.4 | 1.1 | 1.1 |
| AA522311_RIKEN_cDNA_0610038P07_gene | 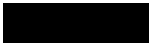 | 1.4 | 1.5 | 1.5 |
| AA967857_brain_protein | 1.0 | −1.5 | 1.2 | 1.1 |
| AA253844_ESTs | 1.48945* | 1.2 | 1.4 | 1.24284* |
| AI122516_microtubule-associated_protein_6 | −1.0 | −1.1 | −1.2 | −1.1 |
| AI430438_RIKEN_cDNA_3110023G01_gene | −1.0 | 1.1 | 1.1 | −1.0 |
| AI550596_stearoyl-Coenzyme_A_desaturase_2 | 1.5 | 1.2 | 1.4 | 1.3 |
| AA200457_ESTs | −1.1 | 1.1 | −1.3 | −1.1 |
| AI324398_glycosylphosphatidylinositol_specific_phospholipase_D1 | −1.3 | 1.2 | −1.6 | −1.6 |
| AA929696_peptidyl_arginine_deiminase,_type_II | −1.3 | 1.0 | −1.4 | 1.1 |
| AA123314_ESTs,_Highly_similar_to_S68176_TOG_pro- | −1.1 | −1.0 | −1.1 | −1.2 |
| tein_[H.sapiens] | | | | |
| AA473684_RIKEN_cDNA_C030002O17_gene | 1.3697+ | 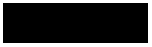 | 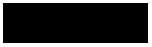 | 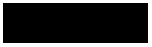 |
| AA518686_ESTs | 1.4 | 1.2 | 1.5 | 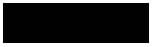 |
| AA412921_RIKEN_cDNA_4921514I20_gene | 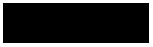 | 1.4 | 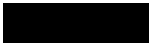 | 1.5 |
| AA051041_potassium_voltage- | 1.10775+ | −1.1 | −1.21672+ | −1.20296+ |
| gated_channel,_subfamily_Q,_member_2 | | | | |
| AI604587_ESTs | 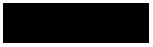 | 1.3 | 1.5 | 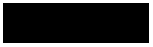 |
| AA059783_Public_domain_EST | 1.1 | 1.2 | 1.1 | −1.1 |
| AA466153_ESTs | 1.2 | 1.3 | 1.1 | −1.33715+ |
| AA222952_zuotin_related_factor_2 | −1.14619+ | 1.0 | 1.0 | 1.0 |
| AI552958_RIKEN_cDNA_1110003E08_gene | 1.1 | 1.1 | 1.2 | 1.2 |
| AA416143_ESTs | −1.1 | 1.0 | −1.1 | 1.2 |
| AI558253_RIKEN_cDNA_1500015J03_gene | 1.4 | 1.5 | 1.4 | 1.39535+ |
| AI553232_ribosomal_protein_S6 | 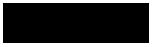 | 1.4 | 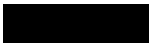 | 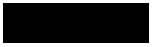 |
| AA511234_ESTs | −1.2 | −1.1 | −1.1 | 1.0 |
| AI550872_RAB11a,_member_RAS_oncogene_family | 1.2 | 1.3 | 1.2 | 1.4 |
| AA839604_ESTs | −1.3 | 1.2 | −1.1 | −1.3 |
| AA120302_ESTs | −1.1 | 1.1 | 1.0 | 1.2 |
| AA412912_ESTs | 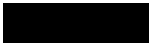 | 1.2 | 1.5 | 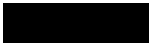 |
| AA003144_ESTs | 1.1 | 1.4 | 1.1 | 1.0 |
| AA387339_ESTs | −1.1 | −1.1 | −1.0 | −1.1 |
| AA738833_solute_carrier_family_1,_member_7 | −1.1 | −1.0 | 1.1 | −1.5 |
| AI893895_cytochrome_P450,_2d9 | 1.1 | −1.0 | 1.1 | 1.1 |
| W30045_eukaryotic_translation_initiation_fac- | 1.0 | −1.0 | −1.1 | −1.2 |
| tor_4E_4E_binding_protein_1 | | | | |
| W59510_elastin | −1.1 | −1.3 | 1.0 | −1.3 |
| AA869219_intelectin | 1.2 | −1.0 | 1.5 | 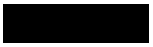 |
| AA822098_apolipoprotein_A-I | −1.2 | −2.1 | −2.5 | −1.6 |
| AI326604_cyclin_F | 1.1 | 1.1 | −1.1 | 1.1 |
| AA671135_myosin_heavy_chain,_cardiac_muscle,_adult | −2.1 | 1.4 | −1.2 | −1.3 |
| AA930278_ESTs | 1.0 | −1.0 | −1.1 | −1.0 |
| AI156185_cystic_fibrosis_transmembrane_con- | 1.17519+ | −1.1 | 1.0 | −1.4 |
| ductance_regulator_homolog | | | | |
| AA792001_RIKEN_cDNA_4931400A14_gene | −1.0 | 1.0 | 1.0 | 1.1 |
| AA672765_Niemann_Pick_type_C1 | 1.0 | −1.3 | −1.1 | −1.2 |
| AA792268_solute_carrier_family_4_(anion_exchanger),_member_1 | −1.1 | −1.2 | −1.6 | −1.7 |
| W53917_serine_hydromethyl_transferase_1_(soluble) | 1.1 | −1.2 | 1.0 | −1.1 |
| AI464601_ESTs | −1.12881+ | −1.0 | −1.07024+ | 1.06265+ |
| AA096815_Public_domain_EST | 1.3 | 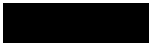 | 1.0 | 1.4 |
| W80245_ESTs | −1.2 | −1.3 | −1.2 | 1.0 |
| AI551578_RIKEN_cDNA_1600013P15_gene | −1.0 | −1.2 | 1.0 | 1.0 |
| AA880295_immunoglobulin_joining_chain | 1.3 | 1.5 | 1.4 | −1.3 |
| AA871838_phospholipase_A2,_group_IIA_(platelets,_synovial_fluid) | 1.5 | 1.2 | 1.3 | 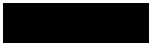 |
| AI529474_carbohydrate_(N-acetylglucosamine_6- | −1.1 | 1.0 | 1.0 | 1.1 |
| O)_sulfotransferase_5 | | | | |
| AA277642_ESTs,_Weakly_similar_to_A53399_L6_anti- gen_-_mouse_[M.musculus] | 1.1 | 1.2 | 1.1 | 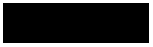 |
| AA667906_custeine-rich_protein_2 | −1.2 | −1.1 | −1.2 | −1.3 |
| AA44443_immunoglobulin_heavy_chain_6_(heavy_chain_IgM) | 1.0 | −1.0 | 1.2 | 1.0 |
| AA065522_RIKEN_cDNA_1700029G01_gene | −1.1 | −1.1 | −1.1 | −1.1 |
| AA656407_troponin_T1,_skeletal,_slow | −1.0 | 1.1 | 1.1 | −1.5 |
| W18463_thioether_S-methyltransferase | −1.7 | 1.2 | −1.0 | −1.2 |
| AA065551_Mus_mus- | 1.0 | −1.2 | 1.0 | −1.0 |
| culus,_clone_IMAGE:3489758,_mRNA,_partial_cds | | | | |
| AA571365_Mus_musculus_MRPS6_mRNA_for_mito- | −1.2 | −1.0 | −1.4 | −1.3 |
| chondrial_ribosomal_protein_S6,_partial_cds | | | | |
| AA146546_ESTs | −1.3 | −1.2 | −1.3 | −1.2 |
| AA028420_small_proline-rich_protein_1A | 1.0 | −1.1 | 1.3 | 1.3 |
| AA718431_RIKEN_cDNA_1700020B19_gene | −1.1 | −1.1 | −1.2 | −1.3 |
| AA109757_solute_carrier_family_22_(organic_cation_transporter)- | −1.1 | 1.2 | −1.1 | −1.1 |
| like_2 | | | | |
| AI605613_RIKEN_cDNA_2210401F01_gene | −1.0 | −1.1 | 1.0 | 1.1 |
| AA671284_troponin_T2,_cardiac | 1.1 | 1.4 | −1.2 | −1.2 |
| AI644912_ESTs,_Weakly_similar_to_A58583_testosterone- | −1.2 | −1.4 | −1.1 | −1.4 |
| resistant_immunity- | | | | |
| associated_protein_IAP38_-_mouse_[M.musculus] | | | | |
| AI181090_cytochrome_450,_subfamily_IV,_B,_polypeptide_1 | −1.2 | 1.1 | −1.1 | −1.1 |
| AA764455_Mus_musculus,_clone_MGC:8241,_mRNA,_com- | −1.1 | −1.1 | −1.1 | 1.1 |
| plete_cds | | | | |
| AA117605_cytotoxic,_granule-associated_RNA-binding_protein_1 | −1.0 | −1.2 | 1.0 | −1.0 |
| AA544749_myosin_Ic | −1.1 | 1.0 | −1.1 | −1.2 |
| AA754696_immuno- | −1.2 | −1.0 | −1.1 | −1.4 |
| globulin_heavy_chain_6_(heavy_chain_of_IgM) | | | | |
| W53837_mitogen_activated_protein_kinase_13 | −1.0 | −1.1 | −1.0 | 1.1 |
| AA600430_myeloblastosis_oncogene-like_2 | −1.1 | −1.1 | −1.3 | 1.0 |
| AA982280_N-myc_(and_STAT)_interactor | 1.1 | −1.3 | −1.0 | 1.2 |
| AA798953_transformation_related_protein_63 | −1.2 | −1.2 | −1.0 | −1.0 |
| AI046576_testis_specific_gene_1 | 1.2 | −1.1 | 1.5 | 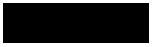 |
| AA881032_Mus_musculus_ETL1_mRNA,_complete_cds | −1.1 | −1.3 | −1.2 | −1.1 |
| AA771566_troponin_C,_fast_skeletal | −3.0 | −1.3 | −1.3 | −1.3 |
| AA545451_gamma-glutamyl_transpeptidase | 1.4 | 1.3 | 1.1 | 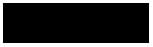 |
| AA458072_adipocyte_complement_related_protein_of_30_kDa | −1.5 | 1.1 | 1.3 | −1.5 |
| AI047912_Mus_musculus_DNA_seg- | 1.30571+ | −1.08385+ | 1.1837+ | −1.0 |
| ment,_Chr_1,_Pasteur_institute_1_(D1Past1),_mRNA | | | | |
| AI385784_pleckstrin_2 | −1.1 | −1.0 | 1.0 | −1.1 |
| AA671573_chemokine_(C—C)_receptor_5 | −1.4 | −1.3 | −1.1 | 1.0 |
| W16358_mucin_1,_transmembrane | 1.1 | 1.1 | −1.2 | 1.0 |
| AI227406_lysosomal_trafficking_regulator | −1.0 | −1.2 | 1.2 | −1.2 |
| AA711611_RIKEN_cDNA_1110015E18_gene | 1.1 | −1.1 | −1.3 | −1.2 |
| AA760185_carbonic_anhydrase_6 | 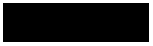 | −1.6 | −1.2 | −1.4 |
| AI552705_NADPH_oxidase_4 | −1.0 | −1.0 | −1.4 | −1.1 |
| AA967824_arginine_vasopressin | −1.3 | 1.4 | −1.2 | −2.3 |
| AI098411_Public_domain_EST | −1.2 | −1.1 | −1.1 | −1.2 |
| AI594906_ESTs | −1.3 | −1.0 | −1.2 | −1.00617+ |
| AA414490_RIKEN_cDNA_1200014D15_gene | −1.1 | −1.1 | 1.1 | 1.0 |
| W13670_amino-terminal_enhancer_of_split | 1.1 | 1.1 | 1.1 | 1.1 |
| AA771229_ESTs,_Weakly_similar_to_zinc_finger_pro- | −2.2 | −1.4 | −2.4 | −1.4 |
| tein_95_[M.musculus] | | | | |
| AA760220_RIKEN_cDNA_2300002G24_gene | −1.0 | −1.1 | −1.2 | −1.1 |
| AI385961_cAMP_responsive_element_modulator | −1.1 | −1.0 | −1.1 | 1.2 |
| AI152670_ESTs | 1.06908+ | −1.1 | 1.1 | −1.38695+ |
| AA739040_lipoprotein_lipase | −1.6 | 1.3 | 1.1 | −1.3 |
| AA004134_centromere_autoantigen_A | −1.1 | −1.2 | −1.1 | 1.0 |
| AA105999_B-cell_src-homology_tyrosine_kinase | −1.0 | −1.1 | −1.3 | 1.0 |
| AA874245_RIKEN_cDNA_1810047J07_gene | −1.0 | 1.1 | 1.2 | 1.1 |
| AA756749_submaxillary_gland_androgen_regulated_protein_1 | 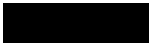 | −1.2 | −1.2 | −1.8 |
| AI894035_proprotein_convertase_subtilisin/kexin_type_5 | 1.0 | −1.2 | −1.1 | −1.2 |
| AI450453_ESTs,_Weakly_similar_to_T33424_hypothetical_pro- | −1.0 | −1.1 | 1.1 | −1.6 |
| tein_M01G5.4_-_Caenorhabditis_elegans_[C.elegans] | | | | |
| AA840053_actinin_alpha_2_assocated_LIM_protein | −1.2 | −1.4 | −1.3 | −1.7 |
| AA497592_ets_variant_gene_6_(TEL_oncogene) | −1.4 | −1.2 | −1.3 | −1.1 |
| AA667301_troponin_I,_cardiac | −2.2 | 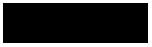 | −1.0 | 1.3 |
| AA517353_nuclear_factor_of_kappa_light_chain_gene_en- | −1.2 | −1.2 | −1.2 | 31 1.0 |
| hancer_in_B-cells_inhibitor,_alpha | | | | |
| AI120822_serine_(or_cysteine)_proteinase_in- | 1.0 | 1.0 | −1.0 | −1.1 |
| hibitor,_clade_G_(C1_inhibitor),_member_1 | | | | |
| AA066016_RIKEN_cDNA_1190002H23_gene | −1.4 | −1.6 | −2.0 | −1.3 |
| AA061920_phospholipase_A2,_group_IIC | 1.1 | 1.0 | −1.2 | 1.3 |
| AA821953_paraoxonase_1 | −1.1 | −1.2 | −1.0 | −1.1 |
| AA416441_gene_rich_cluster,_C8_gene | 1.1 | 1.3 | −1.1 | 1.0 |
| W56995_hemoglobin_X,_alpha- | 1.0 | −1.2 | −4.0 | −1.6 |
| like_embryonic_chain_in_Hba_complex | | | | |
| AA771396_4-hydroxyphenylpyruvic_acid_dioxygenase | 1.3 | −1.2 | 1.1 | 1.5 |
| AA760223_epithelial_membrane_protein_3 | −1.1 | −1.3 | −1.2 | −1.4 |
| W55649_vasodilator-stimulated_phosphoprotein | −1.2 | −1.2 | −1.2 | −1.3 |
| AI019668_prostaglandin_I_receptor_(IP) | 1.5 | −1.1 | 1.29004+ | 1.11434+ |
| AA674177_cytochrome_P450,_2a4 | −1.0 | 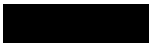 | −1.1 | −1.2 |
| AA437572_inner_centromere_protein | 1.2 | 1.1 | −1.0 | 1.1 |
| AI020539_secretory_leukocyte_protease_inhibitor | −1.0 | −1.1 | −1.1 | −1.4 |
| W83739_DNA_seg- | −1.3 | −1.2 | 1.1 | 1.0 |
| ment,_Chr_2,_Brigham_&_Women's_Genetics_1423_expressed | | | | |
| AA198236_kit_ligand | 1.1 | −1.1 | 1.3 | −1.1 |
| AI451780_prostate_specific_ets_transcription_factor | −1.0 | 1.0 | 1.0 | 1.0 |
| W11251_Fc_receptor,_IgE,_high_affinity_I,_gamma_polypeptide | 1.1 | −1.1 | −1.1 | −1.1 |
| AA691355_Mus_musculus_DNA_cytosine_methyl- | −1.2 | −1.1 | −1.4 | −1.02751+ |
| transferase_mRNA | | | | |
| AI324400_transforming_growth_factor,_beta_receptor_II | 1.3 | 1.1 | 1.0 | 1.0 |
| AA733479_syndecan_1 | −1.1 | −1.1 | 1.0 | −1.2 |
| AA617316_TEA_damain_family_member_2 | −1.3 | −1.3 | −1.3 | −1.1 |
| AA049060_inter-trypsin_inhibitor,_heavy_chain_4 | 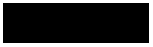 | 1.1 | 1.4 | 1.3 |
| AA759995_procollagen,_type_XVII,_alpha_1 | 1.2 | −1.1 | 1.0 | −1.2 |
| AA028578_keratin_complex_2,_basic,_gene_4 | 1.4 | −1.1 | 1.5 | 1.3 |
| AA755961_insulin-like_growth_factor_2_receptor | −1.3 | −1.1 | −1.1 | −1.1 |
| AA727051_myeloid_leukemia_factor_1 | −1.1 | −1.3 | 1.1 | −1.4 |
| AA288467_glycosylation_dependent_cell_adhesion_molecule_1 | −1.0 | −1.1 | 1.2 | 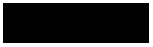 |
| AA444639_sialyltransferase_7_((alpha-N-acetylneuraminyl_2,3-beta- | −1.1 | −1.3 | 1.2 | 1.5 |
| galactosyl-1,3)N-acetyl_galactosamide_alpha-2,6-sialyltransferase)_B | | | | |
| AA545691_thymidine_kinase_1 | 1.3 | 1.0 | −1.1 | 1.1 |
| AA106072_Public_domain_EST | 1.0 | 1.3 | −1.3 | 1.1 |
| AA414110_ESTs | −1.0 | −1.1 | 1.1 | 1.1 |
| AA414112_phosphatidylinositol_glycan,_class_A | −1.0 | −1.0 | 1.2 | 1.2 |
| AI046562_lactate_dehydrogenase_3,_C_chain,_sperm_specific | −1.1 | 1.0 | 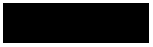 | 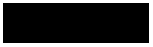 |
| AI120958_RIKEN_cDNA_2700018N07_gene | −1.2 | −1.3 | −1.1 | 1.0 |
| W16012_uteroglobin | −1.2 | 1.1 | −1.2 | −1.3 |
| AA152940_immunoglobulin_kappa_chain_variable_28_(V28) | −1.3 | 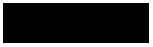 | −1.1 | −2.5 |
| AI326527_preproacrosin | −1.1 | −1.2 | −1.1 | 1.2 |
| AA267626_mitogen_activated_pro- | −1.00038+ | 1.1 | 1.1 | −1.1 |
| tein_kinase_kinase_kinase_kinase_2 | | | | |
| AA607483_proteoglycan,_secretory_granule | 1.1 | −1.2 | −1.1 | 1.4 |
| AA982227_glutathione_S-transferase,_alpha_3 | 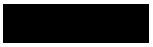 | 1.3 | −1.2 | −1.0 |
| AA220024_trophoblast_specific_protein | 1.4 | 1.1 | 1.2 | 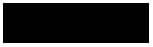 |
| AA106793_plasminogen | 1.0 | 1.0 | −1.4 | −1.1 |
| W08947_non- | −1.8 | −1.3 | −1.9 | −1.2 |
| catalytic_region_of_tyrosine_kinase_adaptor_protein_2 | | | | |
| AI050373_RIKEN_cDNA_4921524P20_gene | −1.7 | −1.1 | −1.2 | −1.2 |
| AA612413_chitinase,_acidic | 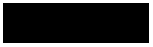 | −1.2 | −1.2 | −1.0 |
| AI327450_phospholipase_A2,_group_IB,_pancreas | −1.4 | −1.4 | −1.1 | −1.0 |
| AA117554_growth_arrest_specific_2 | 1.0 | −1.3 | −1.4 | −1.4 |
| AA529824_fatty_acid_binding_protein_2,_intestinal | 1.4 | 1.1 | 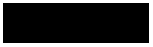 | 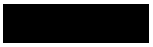 |
| W36635_carbonyl_reductase_2 | 1.0 | 1.2 | 1.5 | 1.1 |
| AI154217_poliovirus_sensitivity | −1.3 | −1.1 | −1.3 | −1.1 |
| AA237607_pyruvate_kinase_liver_and_blood_cell | 1.2 | −1.1 | 1.0 | 1.1 |
| AA600553_claudin_7 | −1.1 | 1.0 | −1.3 | 1.0 |
| AI019575_secreted_frizzled-related_sequence_protein_4 | 1.0 | −1.2 | −1.2 | 1.3 |
| W18281_RIKEN_cDNA_2310012I15_gene | 1.0 | 1.1 | 1.1 | 1.2 |
| AA028346_keratin_complex_1,_acidic,_gene_19 | −1.1 | −1.3 | −1.2 | −1.2 |
| AA061047_secreted_frizzled-related_sequence_protein_2 | −1.1 | −1.0 | 1.1 | −1.5 |
| AA220582_cytochrome_P450,_2f2 | −1.2 | −1.1 | 1.2 | −1.0 |
| AA108495_deoxyribonuclease_I | 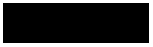 | −2.4 | 1.3 | 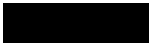 |
| AA655921_neutral-salient_serine_arginine-rich | −1.1 | −1.3 | −1.3 | −1.1 |
| AI159406_casein_gamma | 1.0 | −1.1 | 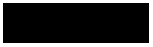 | −1.2 |
| AI326575_glucose-6-phosphatase,_catalytic | 1.5 | 1.1 | 1.2 | 1.5 |
| AI182792_aquaporin_5 | 1.0 | 1.2 | −1.6 | 1.4 |
| AA062457_RIKEN_cDNA_061006F02_gene | 1.1 | −1.3 | −1.1 | 1.3 |
| AA286306_ESTs | 1.1 | 1.09912+ | −1.1 | 1.5 |
| AA549040_histone_acetyltransferase | −1.0 | −1.1 | −1.1 | −1.2 |
| AA656712_myosin,_heavy_polypeptide_1,_skeletal_muscle,_adult | −1.3 | 1.1 | −1.2 | −1.3 |
| AI256888_deleted_azoospermia-like | −1.1 | 1.0 | 1.1 | 1.1 |
| AA106125_cytochrome_450,_2a4 | 1.1 | 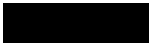 | 1.1 | 1.1 |
| AA064494_Public_domain_EST | 1.3 | 1.0 | 1.4 | 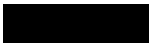 |
| AA066835_corticosteroid_binding_globulin | 1.1 | −1.0 | −1.3 | −2.4 |
| AA106167_Public_domain_EST | −1.2 | −1.1 | −1.1 | −1.2 |
| AI876995_immunoglobulin-associated_alpha | −1.5 | −1.3 | −1.2 | −1.2 |
| AA681602_enolase_3,_beta_muscle | −1.9 | 1.1 | 1.2 | −1.4 |
| W17866_uterine-specific_proline-rich_acidic_protein | −1.3 | −1.3 | −1.5 | 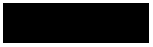 |
| AI060394_ESTs | −1.6 | −1.0 | −1.6 | −1.0 |
| AA864136_lectin,_galactose_binding,_soluble_7 | 1.1 | 1.0 | 1.1 | −1.0 |
| AI552711_creatine_kinase,_muscle | −1.4 | −1.0 | −1.1 | −1.2 |
| AA980349_glutathione_peroxidase_2,_pseudogene_1 | 1.1 | −1.1 | −1.5 | 1.5 |
| AA756171_cysteine-rich_protein_3 | −1.2 | 1.0 | −1.1 | −1.2 |
| AA822346_odorant-binding_protein_Ia | −1.8 | −1.4 | −1.2 | −3.01254* |
| AA276747_ESTs | 1.0 | 1.2 | 1.1 | 1.2 |
| AI390909_gap_junction_membrane_channel_protein_alpha_4 | −1.1 | −1.2 | −1.0 | −1.0 |
| AW496194_immuno- | −1.0 | 1.4 | −1.0 | −2.7 |
| globulin_kappa_chain_variable_20_(V20_family) | | | | |
| W82497_ubiquitin-like_1 | −1.6 | 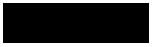 | 1.3 | −1.2 |
| AI425461_ESTs,_Moderately_simi- | −1.2 | −1.2 | −1.2 | −1.1 |
| lar_to_1615347A_ras_p21_GTPase_activating_pro- | | | | |
| tein_[M.musculus] | | | | |
| AA189897_ESTs | 1.1 | −1.1 | 1.2 | −1.00032+ |
| AI664286_tumor_nucrosis_factor_receptor_superfamily,_member_9 | −1.1 | −1.2 | 1.1 | 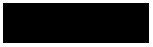 |
| AA823639_RIKEN_cDNA_A930018B01_gene | 1.0 | −1.1 | −1.0 | −1.1 |
| AI594276_transcription_factor_AP-2,_alpha | −1.2 | −1.2 | −1.1 | −1.1 |
| AA122791_histocompatibility_2,_Q_region_locus_7 | −1.3 | −1.0 | −1.3 | −1.2 |
| AA759679_Ia-associated_invariant_chain | −1.9 | −1.0 | −1.3 | −1.4 |
| AA681596_procollagen-lysine,_2-oxoglutarate_5-dioxygenase_1 | −1.0 | 1.1 | −1.0 | −1.1 |
| AA538511_histocompatibility_2,_2,_D_region_locus_1 | −1.0 | −1.0 | 1.0 | −1.1 |
| AA273374_RIKEN_cDNA_1810008K20_gene | −1.2 | −1.1 | 1.1 | 1.1 |
| AA413953_ESTs,_Weakly_similar_to_KIAA0661_pro- | −1.0 | −1.2 | −1.1 | 1.1 |
| tein_[H.sapiens] | | | | |
| W11170_small_inducible_cytokine_A21a_(leucine) | −1.1 | −1.1 | −1.3 | 1.0 |
| AA717019_ATPase,_Ca++_trans- | −1.8 | −1.2 | 1.1 | −1.1 |
| porting,_cardiac_muscle,_fast_twich_1 | | | | |
| AA016759_mini_chromosome_maintenance_defi- | −1.4 | −1.3 | −1.6 | −1.1 |
| cient_6_(S._cercisiae) | | | | |
| W54403_ATPase,_Cu++_transporting,_beta_polypeptide | 1.1 | −1.1 | −1.1 | 1.2 |
| AA145479_ESTs | −1.3 | −1.3 | −1.2 | −1.3 |
| AI903969_sarcoglycan,_beta_(43kD_dystrophin- | −1.3 | −1.4 | 1.0 | −1.2 |
| associated_glycoprotein) | | | | |
| AI226235_gap_junction_membrane_channel_protein_beta_4 | −1.3 | −1.1 | −1.1 | −1.0 |
| AA795319_myosin_light_chain,_phos- | −1.3 | 1.1 | 1.1 | 1.0 |
| phorylatable,_cardiac_ventricles | | | | |
| AI639807_ESTs,_Weakly_similar_to_E6- | 1.11408+ | −1.2 | 1.1 | 1.2 |
| AP_ubiquitin_protein_logase_[M.musculus] | | | | |
| AA212144_forkhead_box_A2 | 1.1 | −1.4 | −1.5 | −1.1 |
| AA871265_Public_domain_EST | 1.2 | 1.3 | 1.4 | −1.7 |
| AA270904_nuclear_receptor_subfamily_1,_group_H,_member_4 | −1.00129+ | 1.1 | 1.1 | 1.29189+ |
| AA738672_Mus_musculus_mRNA_for_thymopsin,_complete_cds | −1.2 | −1.1 | −1.3 | −1.5 |
| W36421_Moloney_leukemia_virus_10 | 1. | −1.1 | −1.1 | 1.1 |
| AI550101_ESTs | −1.3 | −1.3 | −1.2 | −1.2 |
| AA008383_phosphodiesterase_6D,_cGMP-specific,_rod,_delta | 1.1 | −1.1 | 1.0 | −1.2 |
| AI509041_Mus_musculus_Rep- | 1.0 | −1.1 | 1.1 | −1.0 |
| 8,_complete_cds | | | | |
| AA549705_ESTs | −1.0 | −1.3 | −1.2 | −1.4 |
| W18330_tropomysin_2,_beta | 1.1 | −1.1 | −1.3 | −1.4 |
| AA815777_RIKEN_cDNA_1110007F23_gene | 1.0 | 1.1 | −1.2 | −1.5 |
| Q15812_desmin | −1.1 | −1.1 | −1.3 | −1.0 |
| AI595315_ESTs | −1.2 | −1.1 | 1.1 | 1.19019+ |
| AA549305_RIKEN_cDNA_4833427P12_gene | −1.0 | −1.0 | −1.1 | 1.05966+ |
| AA114721_RIKEN_cDNA_5830475I06_gene | −1.4 | −1.1 | −1.16457* | −1.1 |
| AA089188_uromodulin | −1.7 | 1.1 | −1.2 | −1.4 |
| AA839138_CD8_antigen,_alpha_chain | −2.0 | 1.1 | 1.0 | −1.1 |
| AA763276_small_muscle_protein,_X-linked | −1.6 | −1.2 | −2.0 | −1.5 |
| AA474047_ESTs | −1.2 | −1.3 | −1.0 | −1.2 |

COLON

| 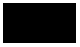 | spl | panc | liv | stom |
|---|---|---|---|---|
| Description | PL-4B_BDEnor | PL-2B_BDEnor | PL-6B_BDEnor | PL-8B_BDEnor |
| 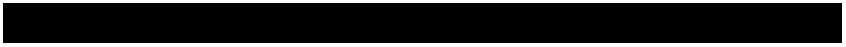 | −1.1 | −1.4 | 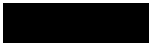 | 1.3 |
| 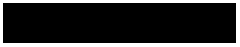 | −1.8 | 1.3 | 1.1 | 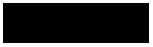 |
| 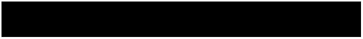 | −1.0 | −1.6 | 1.4 | −1.1 |
| 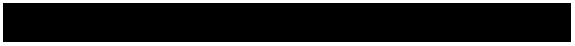 | 1.1 | −1.1 | 1.0 | −1.8 |
| 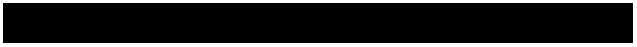 | −1.2 | −1.3 | 1.1 | −1.1 |
| 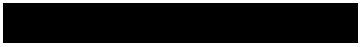 | −1.5 | 1.0 | 1.4 | 1.0 |
| 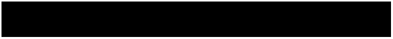 | −1.2 | −1.1 | 1.3 | 1.3 |
| 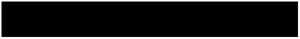 | −1.0 | −1.6 | 1.3 | −1.0 |
| 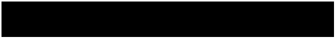 | −1.3 | 1.2 | 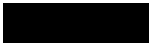 | 1.1 |
| 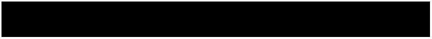 | 1.1 | −1.0 | 1.3 | 1.1 |
| 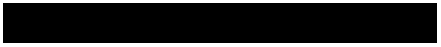 | −1.6 | −1.0 | 1.1 | 1.2 |
| 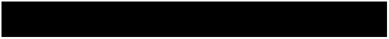 | −1.1 | 1.2 | 1.4 | 1.1 |
| 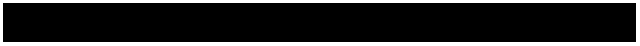 | −1.5 | −1.5 | −1.7 | 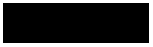 |
| 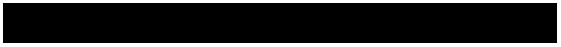 | −1.4 | −1.1 | −1.8 | −1.5 |
| 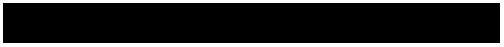 | −1.2 | 1.1 | −1.2 | 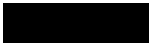 |
| 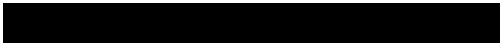 | −1.0 | 1.1 | −1.1 | 1.1 |
| 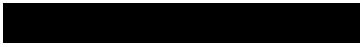 | −1.7 | −1.0 | 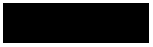 | 1.5 |
| 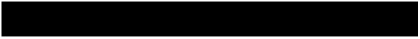 | −1.0 | 1.2 | 1.4 | 1.3 |
| 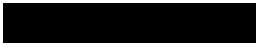 | −2.3 | 1.0 | 1.2 | −1.0 |
| 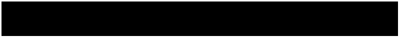 | 1.1 | −2.0 | −1.4 | 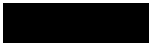 |
| 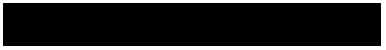 | −1.9 | −1.5 | 1.1 | −2.4 |
| AA869181_defensin_related_cryptdin_6 | −1.8 | −1.7 | −1.5 | 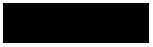 |
| AA546645_cell_division_cycle_2_homolog_(S._pombe)-like_2 | −2.1 | −1.1 | 1.3 | 1.4 |
| AA869219_intelectin | −1.0 | −1.3 | −1.1 | 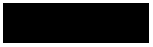 |
| AI465074_mesoderm_specific_transcript | 1.2 | −1.7 | 1.0 | 1.1 |
| AA871914_defensin_related_cryptdin_16 | −1.9 | −1.3 | −1.1 | 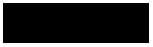 |
| AA982227_glutathione_S-transferase,_alpha_3 | 1.3 | −1.0 | 1.4 | −1.3 |
| AA437876_degenerative_spermatocyte_homolog_(Drosophila) | 1.2 | 1.2 | 1.4 | −1.5 |
| AA023491_RIKEN_cDNA_1600012D06_gene | 1.2 | 1.2 | −1.0 | 1.4 |
| AA024217_Public_domain_EST | −2.5 | 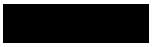 | 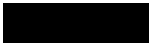 | 1.2 |
| AI613926_twist_gene_homolog,_(Drosophila) | −1.1 | 1.1 | −1.17581+ | 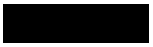 |
| AA108495_deoxyribonuclease_I | 1.1 | −1.8 | −1.1 | 1.2 |
| AI323308_interleukin_3_receptor,_alpha_chain | −1.4 | −1.1 | 1.4 | 1.1 |
| AA727967_serine_(or_crysteine)_proteinase_in- | 1.1 | −1.3 | 1.3 | 1.0 |
| hibitor,_clade_F_(alpha-2_antiplasmin,_pigment_epi- | | | | |
| thelium_derived_factor)_member_1 | | | | |
| AA717167_cytotoxic_T_lymphocyte-associated_protein_2_alpha | 1.4 | −1.8 | −1.3 | 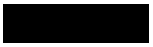 |
| AI595630_RIKEN_cDNA_4930578106_6 | −1.4 | −1.0 | 1.2 | 1.3 |
| AA656712_myosin,_heavy_polypeptide_1,_skeletal_muscle,_adult | 1.0 | −1.6 | 1.1 | 1.2 |
| AA671135_myosin_heavy_chain,_cardiac_muscle,_adult | −1.1 | −1.7 | 1.3 | 1.2 |
| AA725966_sphingosine_kinase_1 | −1.3 | −1.1 | 1.3 | −1.2 |
| AI386288_RIKEN_cDNA_2700043D08_gene | −1.0 | −1.2 | 1.3 | 1.3 |
| AA982240_advanced_glycosylation_end_product-specific_receptor | −1.0 | −1.2 | −1.3 | 1.5 |
| AA895199_ketohexokinase | −1.1 | −1.1 | 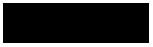 | 1.0 |
| AA270885_parvalbumin | −1.1 | −1.4 | −1.4 | −1.4 |
| AI429054_ESTs | −1.0 | −1.1 | 1.3 | −1.1 |
| AA106834_ESTs,_Weakly_similar_to_EP15_MOUSE_EPI- | −1.3 | 1.1 | 1.0 | −1.2 |
| DERMAL_GROWTH_FACTOR_RECEPTOR_SUB- | | | | |
| STRATE_SUBSTRATE_15_[M.musculus] | | | | |
| AA674392_ESTs,_Moderately_similar_to_A23772_Line- 1_hypothetical_protein_-_mouse_[M.musculus] | −1.2 | −1.5 | 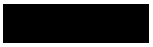 | 1.3 |
| AI892747_glutathione_S-transferase,_alpha_a_(Ya) | −1.1 | −1.3 | 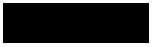 | −2.1 |
| AA067236_fibroblast_growth_factor_receptor_1 | −1.2 | 1.0 | 1.4 | 1.5 |
| AA230638_transcription_factor-like_protein_ODA-10 | −1.2 | 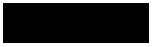 | −1.5 | −1.1 |
| AA871641_deferensin_related_ceyptdin,_related_sequence_2 | −1.0 | 1.3 | −1.4 | 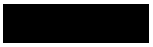 |
| AA067003_glutathione_S-transferase,_mu_1 | 1.1 | 1.2 | 1.0 | −2.0 |
| AA591707_DNA_polymerase_epsilon,_subunit_2 | −1.1 | 1.1 | 1.05228* | 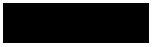 |
| AA546945_tyrosine_3-monooxygenase/tryptdin_5- | −1.3 | 1.3 | 1.4 | −1.1 |
| monooxygenase_activation_protein,_zeta_polypeptide | | | | |
| AA066225_selenoprotein_P_plasma,_1 | 1.2 | 1.4 | 1.2 | −1.7 |
| AI385457_retinol_binding_protein_2,_cellular | −2.0 | −2.1 | −1.6 | −1.9 |
| AA795264_ubiquitin-like_1 | −1.0 | −1.8 | 1.3 | 1.1 |
| AA670943_phosphatidic_acid_phosphatase_2a | 1.1 | −1.1 | 1.4 | 1.2 |
| AI181090_cytochrome_P450,_subfamily_IV_B,_polypeptide_1 | 1.0 | −1.4 | −1.1 | −1.1 |
| AA871838_phospholipase_A2,_group_IIA_(platelets,_synovial_fluid) | −1.1 | −1.0 | −1.2 | 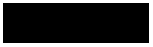 |
| AA763276_small_muscle_protein,_X-linked | −1.7 | −1.4 | 1.1 | 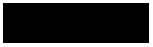 |
| AI385503_cysteine_rich_intestinal_protein | 1.3 | 1.1 | −1.0 | −1.6 |
| AA120401_A_kinase_(PPKA)_anchor_protein_10 | 1.1 | 1.1 | −1.1 | −1.0 |
| AI595611_aquaporin_7 | −1.0 | −1.2 | −1.0 | 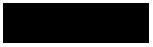 |
| AI892208_protamine_2 | −1.4 | −1.8 | 1.3 | 1.4 |
| AA789873_ATPase,_H+_transporting,_lyso- | −1.5 | 1.4 | 1.3 | 1.0 |
| somal_(vacuolar_proton_pump)_42kD | | | | |
| AA268059_iroquois_related_homobox_3_(Drosophila) | −1.3 | −1.2 | −1.7 | −1.5 |
| AA711681_transaldolase_1 | 1.0 | 1.1 | 1.1 | −1.3 |
| AA145254_SEC61,_gamma_subunit_(S._cerevisiae) | 1.4 | 1.1 | 1.1 | −1.4 |
| AA794074_aldo- | 1.1 | −1.1 | −1.2 | −1.5 |
| keto_reductase_family_1,_member_B1_(aldose_reductase) | | | | |
| AI036487_ESTs | −1.0 | 1.1 | −1.1 | −1.3 |
| W21012_timeless_homolog_(Drosophila) | −1.2 | 1.1 | 1.0 | −1.8 |
| AA138265_RIKEN_cDNA_2010309G21_gene | −1.1 | −2.2 | −1.5 | −1.2 |
| AI121690_RIKEN_cDNA_1300007C21_gene | −2.0 | −1.7 | −1.4 | −1.4 |
| AI876997_carboxyl_ester_lipase | 1.5 | 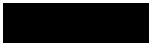 | −1.4 | −2.3 |
| AA106894_carbonic_anhydrase_4 | −1.2 | −1.5 | −1.2 | −1.4 |
| AI158565_guanine_nucleotide_binding_protein_(G_pro- | −1.0 | −1.0 | −1.1 | −1.3 |
| tein),_gamma_10 | | | | |
| AI893892_interferon_regulatory_factor_2 | −1.4 | −1.0 | −1.3 | −1.5 |
| AA238062_CD52_antigen | 1.1 | −1.6 | −1.3 | 1.0 |
| W33651_platelet- | 1.2 | −1.7 | −1.1 | 1.2 |
| activating_factor_acetylhydrolas,_isoform_1b,_alpha1_subunit | | | | |
| AA794055_cysteine_rich_protein | 1.0 | 1.4 | −1.9 | −1.3 |
| AI036381_ninjurin_1 | 1.1 | 1.1 | 1.1 | −1.2 |
| AI326722_lymphocyte_antigen_6_complex,_locus_E | −1.1 | −1.2 | −1.2 | −3.5 |
| AA245754_lysosomal_membrane_glycoprotein_2 | 1.2 | −1.2 | 1.1 | −1.5 |
| AA145895_heat_shock_protein,_70_kDa_1 | 1.1 | −1.1 | −1.0 | 1.2 |
| AI594243_CD8beta_opposite_strand | −1.4 | −1.0 | −2.0 | −1.7 |
| AI390138_RIKEN_cDNA_3930401B19_gene | −1.4 | −10.7 | −1.1 | −2.0 |
| AI120876_CD4a_antigen | 1.3 | 1.2 | −1.5 | −2.8 |
| AI552004_ESTs,_Highly_similar_H- | 1.4 | 1.2 | 1.5 | −1.1 |
| REV_107_PROTEIN_[R.norvegicus] | | | | |
| W15890_guanylate_cyclase_activator_2_(guanylin_2,_in- | 1.0 | 1.0 | −1.3 | 1.2 |
| testinal,_heatstable) | | | | |
| AA444443_immunoglobulin_heavy_chain_6_(heavy_chain_of_IgM) | −1.2 | −1.8 | −1.4 | 1.1 |
| AI894016_complement_component_1,_q_sub- | 1.1 | −1.4 | 1.3 | 1.0 |
| component,_c_polypeptide | | | | |
| AA414831_hypoxia_induced_gene_1 | 1.4 | 1.1 | 1.2 | −1.3 |
| AI892299_RIKEN_cDNA_5830499B15_gene | 1.0 | −1.3 | 1.1 | 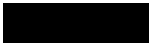 |
| AI322733_2′-5′_oligoadenylate_synthetase_1A | 1.1 | 1.2 | −1.1 | −2.6 |
| AA688795_ESTs | −1.0 | 1.4 | −1.3 | −1.1 |
| AI325323_myocyte_enhancer_factor_2C | −1.3 | −1.0 | 1.2 | −1.3 |
| AA138854_ESTs,_Weakly_similar_to_lyso- | −1.4 | −1.6 | −1.1 | −1.0 |
| phospholipase_I_[M.musculus] | | | | |
| AI876995_immunoglobulin-associated_alpha | −1.1 | −1.8 | −1.2 | −1.6 |
| AI060720_T-cell_specific_GTPase | 1.0 | −1.3 | −1.2 | −1.8 |
| AA675084_elastase_2 | 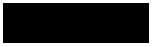 | 1.4 | −1.2 | −2.7 |
| AA123007_2′-5′_oligoadenylate_synthetase-like | 1.1 | −1.4 | −1.3 | −2.2 |
| AA237793_rat_regenerating_islet-derived,_mouse_homolog_1 | 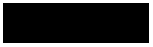 | 1.4 | −1.3 | −3.9 |
| AA688697_distal_intestinal_serine_protease | 1.1 | 1.3 | −1.2 | 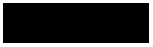 |
| AI037523_transgelin | 1.1 | −1.1 | ×1.2 | −1.5 |
| AI325697_lympocyte_antigen_6_complex | 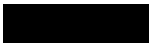 | −1.1 | 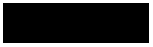 | −1.3 |
| AA237943_sperm_specific_antigen_1 | 1.0 | −1.0 | 1.1 | −1.1 |
| AI605616_ESTs | −1.2 | −1.1 | 1.3 | −1.5 |
| W62520_thiosulfate_sulfurtransferase,_mitochondrial | 1.1 | −1.0 | 1.3 | −1.7 |
| AI036062_histocompatibility_2,_class_II,_locus_DMa | −1.3 | 1.2 | −1.2 | 1.2 |
| AA939788_caspase_7 | −1.1 | −1.0 | 1.0 | 1.0 |
| AA097896_0-6-methylguanine-DNA_methyltransferase | −1.2 | 1.2 | −1.6 | −1.9 |
| AI481911_Mus_musculus,_clone_MGC:6727,_mRNA,_comp- | −1.1 | −2.0 | 31 1.8 | 1.0 |
| lete_cds | | | | |
| AA498574_kaltikrein_6 | 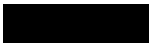 | 1.5 | −1.2 | −1.4 |
| W15001_CD52_antigen | 1.1 | −1.7 | 1.2 | 1.1 |
| AA619407_pancreatitis-associated_protein | 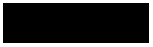 | 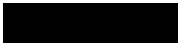 | −1.9 | 1.2 |
| AA575501_ESTs | −1.4 | 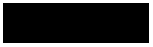 | 1.0 | −1.3 |
| AA684403_ESTs | −1.4 | 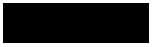 | −2.3 | −2.0 |
| AA458072_adipocyte_complement_related_protein_of_30_kDa | 1.3 | −1.2 | −1.0 | −1.3 |
| AA980349_glutathione_peroxidase_2,_pseudogene_1 | −1.3 | −1.0 | 1.2 | −1.9 |
| W09198_calcium_binding_protein_A6_(calcyclin) | 1.2 | −1.2 | −1.6 | |
| AI386062_carbonic_anhydrase_3 | 1.1 | −1.3 | 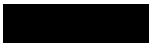 | −1.5 |
| AA880295_immunoglobulin_joining_chain | 1.2 | —1.5 | −1.4 | 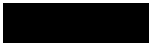 |
| AA068624_hydroxyacid_oxidase_(glycolate_oxidase)_3 | 1.3 | 1.3 | −1.2 | 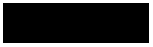 |
| AA146478_M.musculus_mRNA_(3C10)_for_IgA_V- D-J-heavy_chain | −1.3 | 1.0 | −1.6 | 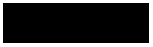 |
| AA050168_proteasome_(prosome,_macropain)_sub- | −1.1 | 1.2 | 1.2 | −1.1 |
| unit,_beta_type_10 | | | | |
| AI451862_RIKEN_cDNA_0910001A18_gene | 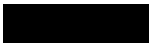 | 1.3 | −1.1 | −2.5 |
| AI386046_trypsin_4 | 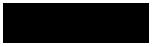 | 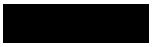 | 1.1 | −1.8 |
| AA386807_RIKEN_cDNA_2310002A12_gene | −1.0 | 1.1 | −1.7 | −1.3 |
| AA822485_proteosome_(prosome,_macropain)_sub- | 1.0 | −1.3 | 1.3 | −1.1 |
| unit,_beta_type_9_(large_multifunctional_protease_2) | | | | |
| AA880322_calponin_1 | −1.2 | −1.4 | −1.1 | −1.1 |
| AA212405_dihydrofolate_reductase | −1.2 | −1.6 | −1.2 | −1.0 |
| AI529513_ESTs,_Moderately_similar_to_F26L_6PF-2-K/ | −1.2 | 1.1 | 1.1 | −1.6 |
| FRU-2,6-P2ASE_LIVE_ISOZYME_[M.musculus] | | | | |
| AA762277_lympocyte_antigen_6_complex,_locus_C | 1.4 | 1.1 | 1.1 | 1.0 |
| AI048040_claudin_4 | −1.6 | 1.5 | −2.1 | −1.9 |
| AI326566_histocompatibility_2,_class_II_antigen_E_alpha | 1.4 | −1.5 | 1.2 | 1.1 |
| W11170_small_inducible_cytokine_A21a_(leucine) | −1.2 | −1.0 | 1.1 | 1.0 |
| AA967824_arginine_vasopressin | 1.4 | −1.6 | −1.2 | −1.1 |
| W08321_inhibitor_of_DNA_binding_1 | 1.1 | −1.1 | 1.1 | −2.0 |
| AI157238_histocompatibility_2,_class_II,_locus_Mb1 | −1.2 | −1.1 | −1.1 | 1.1 |
| AA272836_aquaporin_8 | −1.1 | 1.2 | −1.1 | 1.4 |
| AW496194_immuno- | 1.4 | −1.2 | −1.4 | −1.1 |
| globulin_kappa_chain_variable_20_(V20_family) | | | | |
| AA792785_histocompatibility_2,_class_II,_antigen_E_beta | 1.2 | −1.7 | −1.1 | 1.5 |
| AI428626_ESTs | −1.4 | 1.1 | −2.1 | −1.8 |
| AA871265_Public_domain_EST | 1.3 | −1.4 | −2.0 | 1.4 |
| AA152940_immunoglobulin_kappa_chain_variable_28_(V28) | 1.3 | −1.6 | −1.5 | −1.0 |
| AA754696_immuno- globulin_heavy_chain_6_(heavy_chain_of_IgM) | 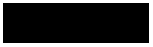 | −1.5 | 1.0 | −1.2 |
| AA790398_ribonuclease_1,_pancreatic | 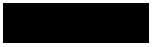 | 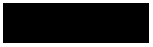 | −1.2 | −5.8 |
| AA072834_RIKEN_cDNA_9030418M05_gene | 1.1 | 1.2 | −1.1 | −2.1 |
| W17930_small_inducible_cytokine_A21a_(leucine) | −1.2 | 1.0 | −1.4 | −1.2 |
| AA242406_CEA-related_cell_adhesion_molecule_1 | −1.1 | −1.1 | 1.0 | 1.4 |
| AI627143_ESTs | −1.0 | −1.1 | 1.1 | −1.3 |
| AA250087_proteosome_(prosome,_macropain)_sub- | 1.1 | −1.5 | 1.1 | −1.9 |
| unit,_beta_type_8_(large_multifunctional_protease_7) | | | | |
| W15809_hemoglobin,_beta_adult_major_chain | 1.5 | −1.2 | 1.2 | −2.9 |
| AA759679_Ia-associated_invariant_chain | −1.0 | −1.8 | −1.6 | −1.9 |
| AA790409_sialytranferase_7_(alpha-N-acetylneuraminyl_2,3- | −1.0 | −1.1 | 1.0 | 1.2 |
| betagalactosyl-1,3)-N-acetyl_galactosaminide_alpha-2,6- | | | | |
| sialytransferase)_F | | | | |
| AI605648_small_proline-rich_protein_2A | −1.0 | −1.1 | −1.4 | −1.3 |
| AA821884_amylase_2,_pancreatic | 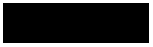 | 1.1 | −1.2 | −2.8 |
| AI386257_calbindin-D9K | −2.2 | −1.3 | 1.2 | 1.5 |
| AA066763_hemoglobin,_beta_adult_major_chain | 1.3 | −1.2 | 1.4 | −4.0 |
| AA106071_hemoglobin,_beta_adult_major_chain | 1.3 | −1.4 | 1.2 | −3.0 |
| AA199011_histocompatibility_2,_class_II_antigen_A,_alpha | −1.1 | −1.3 | 1.1 | 1.0 |
| AA497618_crp-ductin | 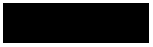 | 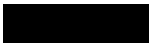 | −1.1 | −1.6 |
| AA717025_Mus_musculus_10_day_old_male_pan- creas_cDNA,_RIKEN_full-length_enriched_li- brary,_clone:1810009A17,_full_insert_sequence | 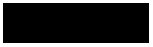 | −1.1 | −1.2 | −2.7 |

COLON

| 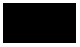 | int | 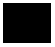 | br | lung |
|---|---|---|---|---|
| Description | PL-3B_BDEnor | PL-5B_BDEnor | PL-1B_BDEnor | PL-7B_BDEnor |
| 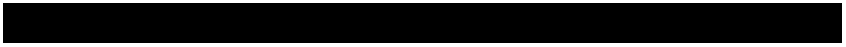 | 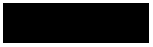 | 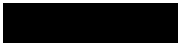 | −1.3 | 1.1 |
| 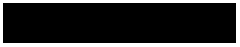 | 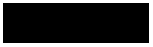 | 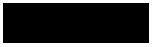 | 1.0 | −2.0 |
| 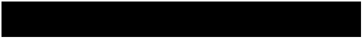 | 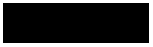 | 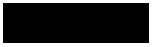 | −1.2 | −1.1 |
| 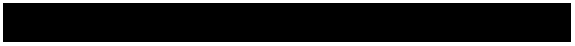 | 1.1 | 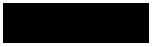 | −1.1 | −1.0 |
| 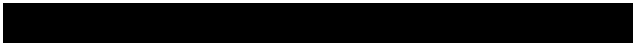 | 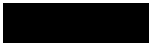 | 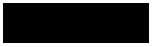 | −1.5 | −1.9 |
| 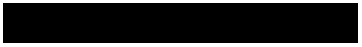 | 1.2 | 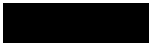 | −1.4 | −2.1 |
| 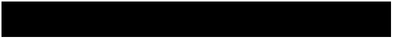 | 1.3 | 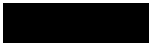 | −1.9 | −2.2 |
| 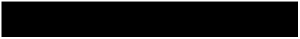 | −1.1 | 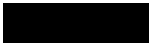 | −1.7 | −1.7 |
| 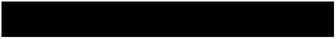 | 1.3 | 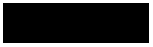 | −1.7 | −2.0 |
| 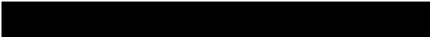 | −1.3 | 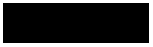 | −1.59207+ | −1.3 |
| 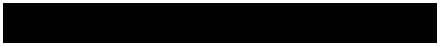 | 1.1 | 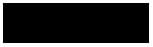 | −1.0 | −1.8 |
| 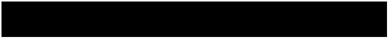 | 1.1 | 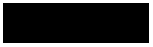 | −1.8 | −1.7 |
| 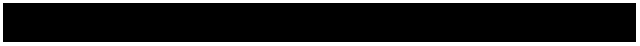 | −1.1 | 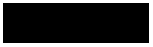 | −2.3 | 1.0 |
| 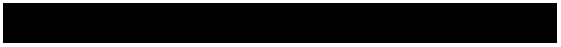 | 1.2 | 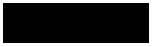 | 1.2 | −1.6 |
| 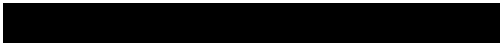 | 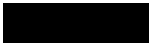 | 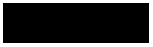 | −1.3 | 1.5 |
| 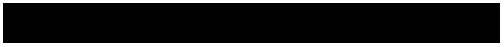 | 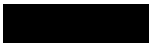 | 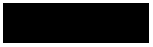 | −1.2 | −1.2 |
| 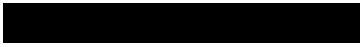 | 1.5 | 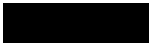 | 1.1 | −1.7 |
| 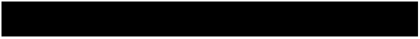 | −1.0 | 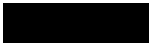 | −1.5 | −1.4 |
| 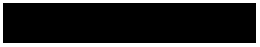 | 1.2 | 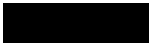 | −1.3 | −2.5 |
| 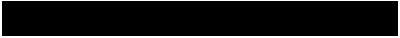 | 1.5 | 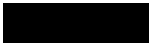 | −1.6 | 1.2 |
| 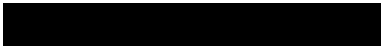 | 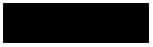 | 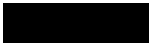 | −1.9 | −9.0 |
| AA869181_defensin_related_cryptdin_6 | 1.5 | 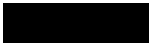 | −1.4 | −1.2 |
| AA546645_cell_division_cycle_2_homolog_(S._pombe)-like_2 | 1.1 | 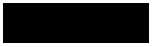 | −1.5 | −3.0 |
| AA869219_intelectin | 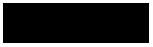 | 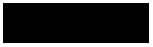 | −1.9 | 1.3 |
| AI465074_mesoderm_specific_transcript | −1.2 | 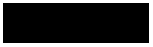 | −1.3 | −1.2 |
| AA871914_defensin_related_cryptdin_16 | 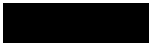 | 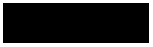 | 1.1 | −1.2 |
| AA982227_glutathione_S-transferase,_alpha_3 | −1.0 | 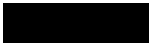 | −2.2 | 1.2 |
| AA437876_degenerative_spermatocyte_homolog_(Drosophila) | 1.3 | 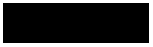 | −1.1 | −1.1 |
| AA023491_RIKEN_cDNA_1600012D06_gene | 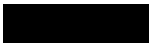 | 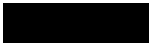 | 1.3 | 1.2 |
| AA024217_Public_domain_EST | 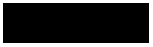 | 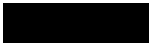 | −1.0 | −4.2 |
| AI613926_twist_gene_homolog,_(Drosophila) | 1.4 | 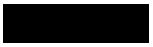 | −1.2 | −1.3 |
| AA108495_deoxyribonuclease_I | 1.4 | 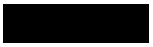 | −2.3 | −1.2 |
| AI323308_interleukin_3_receptor,_alpha_chain | 1.3 | 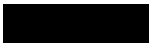 | −1.2 | −1.8 |
| AA727967_serine_(or_crysteine)_proteinase_in- hibitor,_clade_F_(alpha-2_antiplasmin,_pigment_epi- thelium_derived_factor)_member_1 | 1.2 | 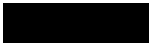 | −1.6 | −1.5 |
| AA717167_cytotoxic_T_lymphocyte-associated_protein_2_alpha | 1.4 | 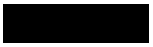 | −1.8 | −1.1 |
| AI595630_RIKEN_cDNA_4930578106_6 | 1.3 | 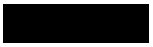 | 1.3 | −1.2 |
| AA656712_myosin,_heavy_polypeptide_1,_skeletal_muscle,_adult | 1.2 | 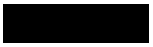 | −2.4 | −1.4 |
| AA671135_myosin_heavy_chain,_cardiac_muscle,_adult | 1.1 | 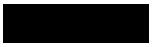 | −1.9 | −1.3 |
| AA725966_sphingosine_kinase_1 | 1.3 | 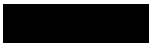 | −1.3 | −1.5 |
| AI386288_RIKEN_cDNA_2700043D08_gene | −1.2 | 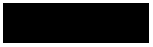 | −1.1 | −1.3 |
| AA982240_advanced_glycosylation_end_product-specific_receptor | 1.3 | 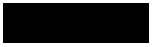 | −1.1 | 1.2 |
| AA895199_ketohexokinase | 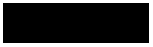 | 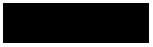 | −1.7 | −1.5 |
| AA270885_parvalbumin | 1.3 | 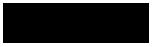 | −1.5 | 1.2 |
| AI429054_ESTs | −1.2 | 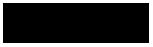 | 1.1 | −1.4 |
| AA106834_ESTs,_Weakly_similar_to_EP15_MOUSE_EPI- DERMAL_GROWTH_FACTOR_RECEPTOR_SUB- STRATE_SUBSTRATE_15_[M.musculus] | 1.0 | 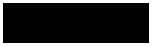 | −1.6 | 1.0 |
| AA674392_ESTs,_Moderately_similar_to_A23772_Line- 1_hypothetical_protein_-_mouse_[M.musculus] | −1.3 | 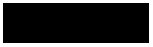 | −1.0 | −1.3 |
| AI892747_glutathione_S-transferase,_alpha_a_(Ya) | −1.7 | 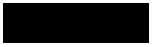 | −1.2 | −1.1 |
| AA067236_fibroblast_growth_factor_receptor_1 | 1.5 | 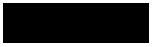 | −1.6 | −1.2 |
| AA230638_transcription_factor-like_protein_ODA-10 | −1.2 | 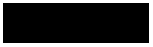 | −1.2 | −1.6 |
| AA871641_deferensin_related_ceyptdin,_related_sequence_2 | 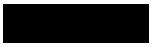 | 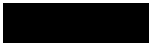 | 1.2 | 1.4 |
| AA067003_glutathione_S-transferase,_mu_1 | 1.5 | 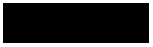 | 1.4 | 1.0 |
| AA591707_DNA_polymerase_epsilon,_subunit_2 | 1.4 | 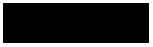 | −1.6 | 1.1 |
| AA546945_tyrosine_3-monooxygenase/tryptdin_5- monooxygenase_activation_protein,_zeta_polypeptide | 1.2 | 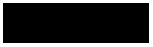 | −1.2 | −2.0 |
| AA066225_selenoprotein_P_plasma,_1 | 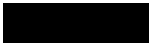 | 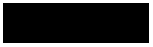 | 1.5 | −1.2 |
| AI385457_retinol_binding_protein_2,_cellular | 1.5 | 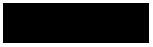 | −1.3 | −1.9 |
| AA795264_ubiquitin-like_1 | 1.1 | 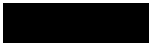 | −1.7 | −1.6 |
| AA670943_phosphatidic_acid_phosphatase_2a | 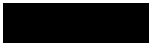 | 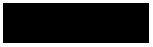 | −1.3 | −1.1 |
| AI181090_cytochrome_P450,_subfamily_IV_B,_polypeptide_1 | 1.1 | 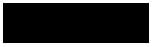 | −1.9 | −1.2 |
| AA871838_phospholipase_A2,_group_IIA_(platelets,_synovial_fluid) | 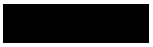 | 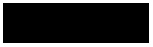 | −1.9 | −1.3 |
| AA763276_small_muscle_protein,_X-linked | 1.2 | 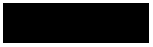 | −4.6 | −2.5 |
| AI385503_cysteine_rich_intestinal_protein | 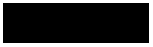 | 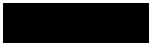 | −1.3 | −1.1 |
| AA120401_A_kinase_(PPKA)_anchor_protein_10 | 1.3 | 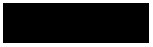 | 1.0 | 1.2 |
| AI595611_aquaporin_7 | 1.3 | 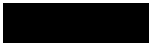 | 1.1 | −1.3 |
| AI892208_protamine_2 | −1.2 | 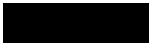 | −1.6 | −1.5 |
| AA789873_ATPase,_H+_transporting,_lyso- somal_(vacuolar_proton_pump)_42kD | 1.4 | 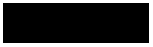 | −1.1 | −1.9 |
| AA268059_iroquois_related_homobox_3_(Drosophila) | −1.1 | −1.9 | 1.2 | −1.4 |
| AA711681_transaldolase_1 | −1.2 | −1.9 | −1.0 | −1.3 |
| AA145254_SEC61,_gamma_subunit_(S._cerevisiae) | −1.7 | −1.9 | −1.2 | −1.3 |
| AA794074_aldo- | −1.0 | −1.9 | −1.1 | 1.0 |
| keto_reductase_family_1,_member_B1_(aldose_reductase) | | | | |
| AI036487_ESTs | −1.9 | −1.9 | 1.0 | 1.0 |
| W21012_timeless_homolog_(Drosophila) | −1.1 | −1.9 | 1.1 | −1.3 |
| AA138265_RIKEN_cDNA_2010309G21_gene | −1.0 | −1.9 | −1.1 | 1.1 |
| AI121690_RIKEN_cDNA_1300007C21_gene | −1.4 | −1.9 | −1.2 | −1.1 |
| AI876997_carboxyl_ester_lipase | −8.9 | −1.9 | −1.3 | −1.4 |
| AA106894_carbonic_anhydrase_4 | 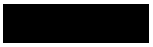 | −1.9 | −1.5 | 1.0 |
| AI158565_guanine_nucleotide_binding_protein_(G_pro- | −1.6 | −1.9 | 1.2 | −1.5 |
| tein),_gamma_10 | | | | |
| AI893892_interferon_regulatory_factor_2 | 1.1 | −1.9 | −1.3 | −1.2 |
| AA238062_CD52_antigen | −1.1 | −1.9 | −1.6 | −1.3 |
| W33651_platelet- | 1.3 | −1.9 | −1.1 | 1.2 |
| activating_factor_acetylhydrolas,_isoform_1b,_alpha1_subunit | | | | |
| AA794055_cysteine_rich_protein | 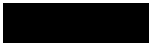 | −2.0 | 1.3 | −1.0 |
| AI036381_ninjurin_1 | 1.1 | −2.0 | 1.1 | −1.4 |
| AI326722_lymphocyte_antigen_6_complex,_locus_E | −1.1 | −2.0 | 1.1 | −1.1 |
| AA245754_lysosomal_membrane_glycoprotein_2 | −1.6 | −2.0 | 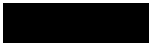 | −1.0 |
| AA145895_heat_shock_protein,_70_kDa_1 | −1.8 | −2.0 | −1.7 | 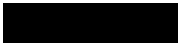 |
| AI594243_CD8beta_opposite_strand | 1.1 | 2.0 | 1.2 | −1.2 |
| AI390138_RIKEN_cDNA_3930401B19_gene | −1.2 | −2.1 | −1.3 | −1.2 |
| AI120876_CD4a_antigen | −1.7 | −2.1 | 1.1 | −1.2 |
| AI552004_ESTs,_Highly_similar_H- | −1.3 | −2.1 | 1.5 | 1.0 |
| REV_107_PROTEIN_[R.norvegicus] | | | | |
| W15890_guanylate_cyclase_activator_2_(guanylin_2,_in- testinal,_heatstable) | 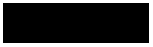 | −2.1 | −1.5 | −1.1 |
| AA444443_immunoglobulin_heavy_chain_6_(heavy_chain_of_IgM) | 1.2 | −2.1 | −1.9 | 1.1 |
| AI894016_complement_component_1,_q_sub- | 1.3 | −2.1 | 1.1 | −1.1 |
| component,_c_polypeptide | | | | |
| AA414831_hypoxia_induced_gene_1 | −1.4 | −2.1 | 1.1 | −1.1 |
| AI892299_RIKEN_cDNA_5830499B15_gene | 1.3 | −2.1 | −1.8 | −1.0 |
| AI322733_2′-5′_oligoadenylate_synthetase_1A | 1.1 | −2.1 | −1.1 | 1.0 |
| AA688795_ESTs | 1.1 | −2.1 | −1.1 | 1.3 |
| AI325323_myocyte_enhancer_factor_2C | −1.7 | −2.1 | 1.2 | 1.2 |
| AA138854_ESTs,_Weakly_similar_to_lyso- | −2.3 | −2.1 | 1.1 | −1.7 |
| phospholipase_I_[M.musculus] | | | | |
| AI876995_immunoglobulin-associated_alpha | −1.1 | −2.1 | −2.4 | −1.2 |
| AI060720_T-cell_specific_GTPase | −1.1 | −2.1 | 1.2 | −1.4 |
| AA675084_elastase_2 | −21.7 | −2.2 | 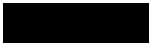 | −1.9 |
| AA123007_2′-5′_oligoadenylate_synthetase-like | −1.1 | −2.2 | −1.1 | 1.3 |
| AA237793_rat_regenerating_islet-derived,_mouse_homolog_1 | −11.8 | −2.2 | −1.1 | 1.3 |
| AA688697_distal_intestinal_serine_protease | 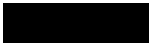 | −2.2 | 1.3 | 1.5 |
| AI037523_transgelin | 1.1 | −2.2 | −1.6 | −1.2 |
| AI325697_lympocyte_antigen_6_complex | 1.1 | −2.2 | −1.3 | −1.1 |
| AA237943_sperm_specific_antigen_1 | −1.2 | −2.2 | 1.0 | 1.0 |
| AI605616_ESTs | −1.9 −2.3 | −1.4 | −1.4 | |
| W62520_thiosulfate_sulfurtransferase,_mitochondrial | −1.3 | −2.3 | −1.2 | −1.3 |
| AI036062_histocompatibility_2,_class_II,_locus_DMa | 1.1 | −2.3 | 1.0 | −1.1 |
| AA939788_caspase_7 | −1.0 | −2.3 | −1.4 | −1.2 |
| AA097896_0-6-methylguanine-DNA_methyltransferase | 1.3 | −2.3 | 1.0 | −1.1 |
| AI481911_Mus_musculus,_clone_MGC:6727,_mRNA,_comp- | −1.8 | −2.3 | −1.5 | −2.4 |
| lete_cds | | | | |
| AA498574_kaltikrein_6 | −2.7 | −2.3 | −1.2 | 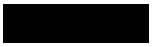 |
| W15001_CD52_antigen | 1.1 | −2.3 | −1.2 | −1.2 |
| AA619407_pancreatitis-associated_protein | 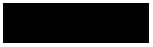 | −2.4 | −1.1 | −1.3 |
| AA575501_ESTs | 1.2 | −2.4 | 1.3 | −1.6 |
| AA684403_ESTs | 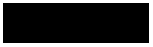 | −2.4 | 1.5 | −1.0 |
| AA458072_adipocyte_complement_related_protein_of_30_kDa | 1.0 | −2.4 | −2.0 | −1.1 |
| AA980349_glutathione_peroxidase_2,_pseudogene_1 | 1.1 | −2.4 | −2.5 | −1.5 |
| W09198_calcium_binding_protein_A6_(calcyclin) | −1.9 | −2.4 | 1.1 | 1.1 |
| AI386062_carbonic_anhydrase_3 | 1.1 | −2.7 | −1.7 | −2.1 |
| AA880295_immunoglobulin_joining_chain | −2.2 | −2.4 | −1.9 | −1.1 |
| AA068624_hydroxyacid_oxidase_(glycolate_oxidase)_3 | 1.32102* | −2.4 | 1.2 | 1.4 |
| AA146478_M.musculus_mRNA_(3C10)_for_IgA_V- | −2.0 | −2.4 | −1.1 | −1.4 |
| D-J-heavy_chain | | | | |
| AA050168_proteasome_(prosome,_macropain)_sub- | −1.1 | −2.5 | −1.0 | −1.3 |
| unit,_beta_type_10 | | | | |
| AI451862_RIKEN_cDNA_0910001A18_gene | −14.7 | −2.6 | 1.2 | −1.3 |
| AI386046_trypsin_4 | −15.9 | −2.6 | 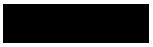 | −1.5 |
| AA386807_RIKEN_cDNA_2310002A12_gene | −1.2 | −2.6 | −1.2 | −1.2 |
| AA822485_proteosome_(prosome,_macropain)_sub- | −1.1 | −2.6 | −1.1 | −1.1 |
| unit,_beta_type_9_(large_multifunctional_protease_2) | | | | |
| AA880322_calponin_1 | 1.1 | −2.6 | −1.7 | −2.1 |
| AA212405_dihydrofolate_reductase | −2.5 | −2.6 | 1.1 | −1.4 |
| AI529513_ESTs,_Moderately_similar_to_F26L_6PF-2-K/ FRU-2,6-P2ASE_LIVE_ISOZYME_[M.musculus] | 1.1 | −2.6 | 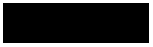 | 1.3 |
| AA762277_lympocyte_antigen_6_complex,_locus_C | −1.2 | −2.6 | 1.2 | −1.3 |
| AI048040_claudin_4 | 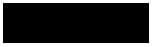 | −2.7 | 1.5 | −1.1 |
| AI326566_histocompatibility_2,_class_II_antigen_E_alpha | 1.5 | −2.7 | −1.1 | 1.2 |
| W11170_small_inducible_cytokine_A21a_(leucine) | 1.3 | −2.7 | −2.8 | −1.1 |
| AA967824_arginine_vasopressin | −1.9 | −2.7 | −2.0 | −1.4 |
| W08321_inhibitor_of_DNA_binding_1 | −1.1 | −2.7 | −1.6 | −1.1 |
| AI157238_histocompatibility_2,_class_II,_locus_Mb1 | 1.2 | −2.8 | −1.2 | −1.2 |
| AA272836_aquaporin_8 | 1.4 | −2.8 | 1.2 | 1.0 |
| AW496194_immuno- | −1.9 | −2.9 | −2.6 | −1.6 |
| globulin_kappa_chain_variable_20_(V20_family) | | | | |
| AA792785_histocompatibility_2,_class_II,_antigen_E_beta | 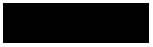 | −2.9 | −1.2 | 1.2 |
| AI428626_ESTs | 1.3 | −3.0 1.2 | −1.2 | |
| AA871265_Public_domain_EST | −1.7 | −3.0 | −3.1 | −1.3 |
| AA152940_immunoglobulin_kappa_chain_variable_28_(V28) | −2.1 | −3.1 | −2.2 | −1.3 |
| AA754696_immuno- | −1.9 | −3.2 | −1.9 | −1.0 |
| globulin_heavy_chain_6_(heavy_chain_of_IgM) | | | | |
| AA790398_ribonuclease_1,_pancreatic | −16.6 | −3.3 | 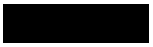 | −1.9 |
| AA072834_RIKEN_cDNA_9030418M05_gene | 1.13744* | −3.1 | −1.1 | −1.1 |
| W17930_small_inducible_cytokine_A21a_(leucine) | 1.2 | −3.3 | −1.3 | −1.0 |
| AA242406_CEA-related_cell_adhesion_molecule_1 | 1.5 | −3.4 | 1.2 | −1.0 |
| AI627143_ESTs | −2.5 | −3.6 | 1.2 | −1.1 |
| AA250087_proteosome_(prosome,_macropain)_sub- | 1.0 | −3.6 | −1.5 | −1.2 |
| unit,_beta_type_8_(large_multifunctional_protease_7) | | | | |
| W15809_hemoglobin,_beta_adult_major_chain | 1.3 | −3.7 | 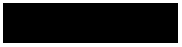 | −1.2 |
| AA759679_Ia-associated_invariant_chain | 1.5 | −3.7 | −2.8 | −1.0 |
| AA790409_sialytranferase_7_(alpha-N-acetylneuraminyl_2,3- | 1.3 | −3.9 | 1.2 | −1.1 |
| betagalactosyl-1,3)-N-acetyl_galactosaminide_alpha-2,6- | | | | |
| sialytransferase)_F | | | | |
| AI605648_small_proline-rich_protein_2A | −5.5 | −3.9 | 1.0 | −1.2 |
| AA821884_amylase_2,_pancreatic | −32.0 | −4.0 | 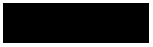 | −1.2 |
| AI386257_calbindin-D9K | −53.5 | −4.1 | 1.0 | −1.2 |
| AA066763_hemoglobin,_beta_adult_major_chain | 1.1 | −4.2 | 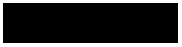 | −1.1 |
| AA106071_hemoglobin,_beta_adult_major_chain | −1.1 | −4.3 | 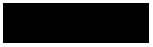 | 1.1 |
| AA199011_histocompatibility_2,_class_II_antigen_A,_alpha | 1.3 | −4.5 | 1.2 | 1.1 |
| AA497618_crp-ductin | −6.1 | −5.0 | 1.5 | −1.2 |
| AA717025_Mus_musculus_10_day_old_male_pan- creas_cDNA,_RIKEN_full-length_enriched_li- brary,_clone:1810009A17,_full_insert_sequence | −25.5 | −5.2 | 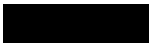 | −1.6 |

COLON

| 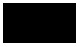 | blad | kidn | pl/ut | mam |
|---|---|---|---|---|
| Description | PL-9B_BDEnor | PL-10B_BDEnor | PL-11B_BDEnor | PL-12B_BDEnor |
| 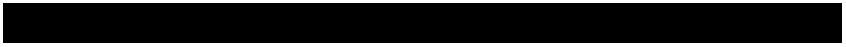 | 1.3 | 1.2 | 1.1 | 1.2 |
| 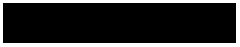 | −1.3 | −1.2 | −1.7 | 1.0 |
| 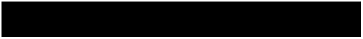 | 1.0 | 1.0 | 1.2 | 1.1. |
| 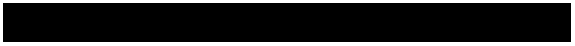 | 1.4 | −1.2 | −1.0 | −1.1 |
| 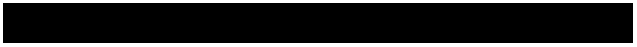 | −1.2 | 1.2 | −1.3 | 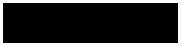 |
| 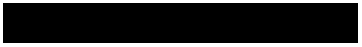 | −1.5 | −1.3 | −1.7 | −1.3 |
| 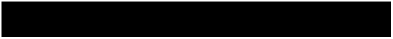 | −1.2 | −1.3 | −1.4 | −1.3 |
| 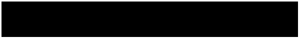 | −1.2 | 1.2 | −1.2 | −1.3 |
| 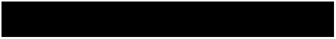 | −1.3 | −1.2 | −1.5 | −1.1 |
| 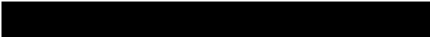 | 1.0 | −1.1 | 1.0 | 1.2 |
| 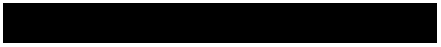 | −1.4 | −1.2 | −1.8 | −1.3 |
| 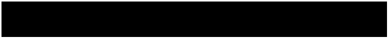 | −1.3 | 1.1 | −1.3 | −1.1 |
| 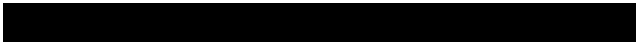 | 1.4 | 1.1 | 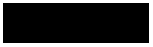 | 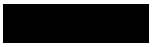 |
| 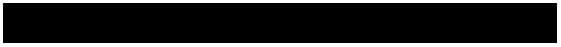 | −1.3 | −1.5 | −1.2 | −1.1 |
| 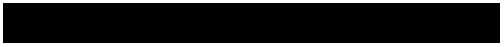 | 1.1 | 1.0 | 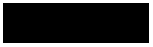 | 1.5 |
| 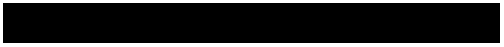 | −1.1 | −1.2 | −1.2 | −1.5 |
| 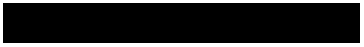 | −1.5 | −1.1 | −1.6 | −1.1 |
| 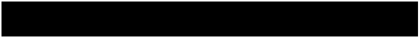 | −1.3 | −1.2 | −1.5 | −1.0 |
| 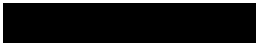 | −2.1 | −1.4 | −2.6 | −1.4 |
| 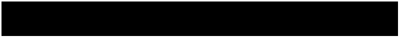 | 1.1 | 1.1 | 1.5 | 1.2 |
| 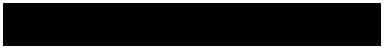 | −1.2 | −2.1 | −2.5 | −1.6 |
| AA869181_defensin_related_cryptdin_6 | 1.1 | −1.2 | 1.4 | 1.3 |
| AA546645_cell_division_cycle_2_homolog_(S._pombe)-like_2 | −2.3 | −1.5 | −2.4 | −1.4 |
| AA869219_intelectin | 1.2 | −1.0 | 1.5 | 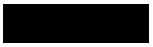 |
| AI465074_mesoderm_specific_transcript | 1.1 | −1.2 | 1.0 | −1.6 |
| AA871914_defensin_related_cryptdin_16 | 1.2 | −1.1 | 1.1 | −1.0 |
| AA982227_glutathione_S-transferase,_alpha_3 | 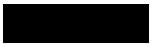 | 1.3 | −1.2 | −1.0 |
| AA437876_degenerative_spermatocyte_homolog_(Drosophila) | −1.1 | 1.2 | −1.2 | −1.2 |
| AA023491_RIKEN_cDNA_1600012D06_gene | 1.4 | −1.0 | −1.5 | 1.4 |
| AA024217_Public_domain_EST | −1.9 | −1.2 | −2.3 | 1.1 |
| AI613926_twist_gene_homolog,_(Drosophila) | 1.1 | 1.0 | −1.2 | −1.0 |
| AA108495_deoxyribonuclease_I | 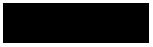 | −2.4 | 1.3 | 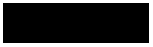 |
| AI323308_interleukin_3_receptor,_alpha_chain | −1.7 | −1.3 | −1.7 | −1.3 |
| AA727967_serine_(or_crysteine)_proteinase_in- | −1.2 | 1.1 | −1.3 | −1.6 |
| hibitor,_clade_F_(alpha-2_antiplasmin,_pigment_epi- | | | | |
| thelium_derived_factor)_member_1 | | | | |
| AA717167_cytotoxic_T_lymphocyte-associated_protein_2_alpha | 1.5 | −1.4 | −1.5 | 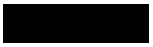 |
| AI595630_RIKEN_cDNA_4930578106_6 | −1.1 | 1.0 | −1.2 | −1.0 |
| AA656712_myosin,_heavy_polypeptide_1,_skeletal_muscle,_adult | −1.3 | 1.1 | −1.2 | −1.3 |
| AA671135_myosin_heavy_chain,_cardiac_muscle,_adult | −2.1 | 1.4 | −1.2 | −1.3 |
| AA725966_sphingosine_kinase_1 | −1.1 | −1.0 | −1.3 | −1.2 |
| AI386288_RIKEN_cDNA_2700043D08_gene | 1.1 | 1.2 | −1.1 | −1.0 |
| AA982240_advanced_glycosylation_end_product-specific_receptor | 1.0 | 1.1 | 1.2 | 1.3 |
| AA895199_ketohexokinase | −1.1 | 1.1 | −1.1 | −1.1 |
| AA270885_parvalbumin | 1.1 | 1.2 | 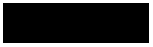 | −1.2 |
| AI429054_ESTs | −1.2 | −1.0 | −1.5 | −1.4 |
| AA106834_ESTs,_Weakly_similar_to_EP15_MOUSE_EPI- | 1.1 | 1.1 | −1.1 | 1.0 |
| DERMAL_GROWTH_FACTOR_RECEPTOR_SUB- | | | | |
| STRATE_SUBSTRATE_15_[M.musculus] | | | | |
| AA674392_ESTs,_Moderately_similar_to_A23772_Line- | −1.3 | −1.3 | 1.2 | −1.1 |
| 1_hypothetical_protein_-_mouse_[M.musculus] | | | | |
| AI892747_glutathione_S-transferase,_alpha_a_(Ya) | 1.4 | 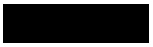 | −1.1 | 1.1 |
| AA067236_fibroblast_growth_factor_receptor_1 | 1.1 | 1.0 | −1.1 | −1.0 |
| AA230638_transcription_factor-like_protein_ODA-10 | −1.1 | −1.1 | −1.3 | −1.5 |
| AA871641_deferensin_related_ceyptdin,_related_sequence_2 | 1.2 | 1.1 | 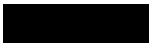 | 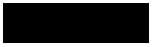 |
| AA067003_glutathione_S-transferase,_mu_1 | 1.2 | 1.4 | −1.3 | −1.2 |
| AA591707_DNA_polymerase_epsilon,_subunit_2 | 1.1 | 1.1 | −1.1 | 1.1 |
| AA546945_tyrosine_3-monooxygenase/tryptdin_5- | −1.5 | −1.2 | −1.8 | −1.1 |
| monooxygenase_activation_protein,_zeta_polypeptide | | | | |
| AA066225_selenoprotein_P_plasma,_1 | −1.1 | 1.2 | −1.6 | −1.1 |
| AI385457_retinol_binding_protein_2,_cellular | −1.6 | −1.9 | −1.5 | −1.7 |
| AA795264_ubiquitin-like_1 | −1.3 | 1.1 | −1.1 | −1.2 |
| AA670943_phosphatidic_acid_phosphatase_2a | 1.0 | 1.0 | 1.1 | −1.0 |
| AI181090_cytochrome_P450,_subfamily_IV_B,_polypeptide_1 | −1.2 | 1.1 | −1.1 | −1.1 |
| AA871838_phospholipase_A2,_group_IIA_(platelets,_synovial_fluid) | 1.5 | 1.2 | 1.3 | 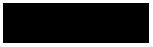 |
| AA763276_small_muscle_protein,_X-linked | −1.6 | −1.2 | −2.0 | −1.5 |
| AI385503_cysteine_rich_intestinal_protein | 1.0 | −1.0 | −1.0 | 1.1 |
| AA120401_A_kinase_(PPKA)_anchor_protein_10 | 1.1 | 1.1 | 1.2 | 1.1 |
| AI595611_aquaporin_7 | 1.1 | 1.1 | 1.0 | −1.0 |
| AI892208_protamine_2 | −1.7 | −1.2 | −1.3 | −1.3 |
| AA789873_ATPase,_H+_transporting,_lyso- | −1.5 | −1.0 | −1.9 | −1.2 |
| somal_(vacuolar_proton_pump)_42kD | | | | |
| AA268059_iroquois_related_homobox_3_(Drosophila) | −1.2 | −1.2 | −1.2 | −1.0 |
| AA711681_transaldolase_1 | −1.0 | 1.0 | −1.1 | −1.0 |
| AA145254_SEC61,_gamma_subunit_(S._cerevisiae) 1.1 | −1.0 | −1.1 | −1.0 | |
| AA794074_aldo- | −1.1 | 1.0 | 1.1 | −1.2 |
| keto_reductase_family_1,_member_B1_(aldose_reductase) | | | | |
| AI036487_ESTs | −1.0 | −1.3 | 1.1 | 1.0 |
| W21012_timeless_homolog_(Drosophila) | −1.2 | −1.0 | −1.3 | −1.1 |
| AA138265_RIKEN_cDNA_2010309G21_gene | −1.3 | −1.0 | −1.0 | −2.4 |
| AI121690_RIKEN_cDNA_1300007C21_gene | −1.7 | −1.5 | 1.1 | 1.2 |
| AI876997_carboxyl_ester_lipase | −1.1 | 1.1 | 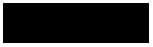 | 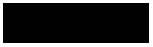 |
| AA106894_carbonic_anhydrase_4 | −1.2 | 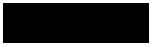 | −1.2 | 1.2 |
| AI158565_guanine_nucleotide_binding_protein_(G_pro- | −1.3 | −1.3 | −1.1 | −1.3 |
| tein),_gamma_10 | | | | |
| AI893892_interferon_regulatory_factor_2 | −1.3 | −1.2 | −1.1 | −1.1 |
| AA238062_CD52_antigen | −2.6 | −1.1 | −1.2 | −1.1 |
| W33651_platelet- | 1.4 | 1.3 | 1.2 | 1.1 |
| activating_factor_acetylhydrolas,_isoform_1b,_alpha1_subunit | | | | |
| AA794055_cysteine_rich_protein | −1.4 | −1.0 | −1.3 | 1.1 |
| AI036381_ninjurin_1 | −1.1 | −1.2 | −1.2 | −1.2 |
| AI326722_lymphocyte_antigen_6_complex,_locus_E | −1.1 | −1.4 | 1.1 | −1.2 |
| AA245754_lysosomal_membrane_glycoprotein_2 | 1.2 | −1.1 | 1.1 | 1.0 |
| AA145895_heat_shock_protein,_70_kDa_1 | 1.1 | 1.1 | −1.0 | −1.1 |
| AI594243_CD8beta_opposite_strand | −1.1 | −1.2 | −1.1 | −1.2 |
| AI390138_RIKEN_cDNA_3930401B19_gene | 1.5 | −1.9 | −1.3 | −1.1 |
| AI120876_CD4a_antigen | −1.0 | −1.0 | −1.4 | 1.1 |
| AI552004_ESTs,_Highly_similar_H- | −1.0 | −1.0 | 1.0 | −1.2 |
| REV_107_PROTEIN_[R.norvegicus] | | | | |
| W15890_guanylate_cyclase_activator_2_(guanylin_2,_in- | −1.2 | −1.0 | 1.1 | 1.4 |
| testinal,_heatstable) | | | | |
| AA444443_immunoglobulin_heavy_chain_6_(heavy_chain_of_IgM) | 1.0 | −1.0 | 1.2 | 1.0 |
| AI894016_complement_component_1,_q_sub- | −1.1 | 1.1 | −1.2 | −1.2 |
| component,_c_polypeptide | | | | |
| AA414831_hypoxia_induced_gene_1 | 1.3 | −1.1 | 1.1 | −1.2 |
| AI892299_RIKEN_cDNA_5830499B15_gene | 1.5 | 1.2 | 1.2 | 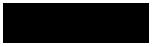 |
| AI322733_2′-5′_oligoadenylate_synthetase_1A | −1.2 | 1.1 | −1.2 | 1.1 |
| AA688795_ESTs | −1.0 | −1.1 | 1.1 | 1.2 |
| AI325323_myocyte_enhancer_factor_2C | −1.1 | −1.0 | −1.1 | −1.2 |
| AA138854_ESTs,_Weakly_similar_to_lyso- | −1.0 | 1.4 | −1.1 | 1.2 |
| phospholipase_I_[M.musculus] | | | | |
| AI876995_immunoglobulin-associated_alpha | −1.5 | −1.3 | −1.2 | −1.2 |
| AI060720_T-cell_specific_GTPase | −1.4 | −1.2 | −1.0 | −1.0 |
| AA675084_elastase_2 | −4.0 | −1.4 | −1.2 | −1.4 |
| AA123007_2′-5′_oligoadenylate_synthetase-like | −1.1 | −1.1 | −1.1 | −1.1 |
| AA237793_rat_regenerating_islet-derived,_mouse_homolog_1 | −2.7 | −1.2 | −1.2 | −1.2 |
| AA688697_distal_intestinal_serine_protease | 1.2 | 1.0 | 1.5 | 1.3 |
| AI037523_transgelin | −1.4 | 1.0 | −1.7 | −1.0 |
| AI325697_lympocyte_antigen_6_complex | 1.4 | 1.1 | 1.1 | −1.1 |
| AA237943_sperm_specific_antigen_1 | 1.1 | 1.1 | 1.0 | 1.0 |
| AI605616_ESTs | −1.2 | −1.2 | −1.5 | −1.1 |
| W62520_thiosulfate_sulfurtransferase,_mitochondrial | −1.0 | −1.3 | −1.2 | −1.2 |
| AI036062_histocompatibility_2,_class_II,_locus_DMa | −1.3 | −1.0 | −1.0 | −1.1 |
| AA939788_caspase_7 | −1.2 | 1.0 | −1.0 | −1.1 |
| AA097896_0-6-methylguanine-DNA_methyltransferase | −1.1 | −1.2 | −1.1 | −1.1 |
| AI481911_Mus_musculus,_clone_MGC:6727,_mRNA,_comp- lete_cds | −1.4 | 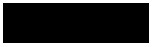 | −1.4 | −1.4 |
| AA498574_kaltikrein_6 | 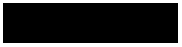 | 1.1 | 1.0 | 1.3 |
| W15001_CD52_antigen | −2.5 | 1.1 | −1.1 | −1.0 |
| AA619407_pancreatitis-associated_protein | 1.0 | −1.2 | 1.3 | 1.1 |
| AA575501_ESTs | −1.5 | −1.2 | −1.3 | −1.1 |
| AA684403_ESTs | −1.3 | −1.3 | −1.1 | −1.1 |
| AA458072_adipocyte_complement_related_protein_of_30_kDa | −1.5 | 1.1 | 1.3 | −1.5 |
| AA980349_glutathione_peroxidase_2,_pseudogene_1 | 1.1 | −1.1 | −1.5 | 1.5 |
| W09198_calcium_binding_protein_A6_(calcyclin) | 1.0 | 1.2 | −1.2 | 1.0 |
| AI386062_carbonic_anhydrase_3 | −1.3 | −1.2 | −1.6 | −1.5 |
| AA880295_immunoglobulin_joining_chain | 1.3 | 1.5 | 1.4 | −1.3 |
| AA068624_hydroxyacid_oxidase_(glycolate_oxidase)_3 | 1.3 | 1.1 | 1.3 | 1.5 |
| AA146478_M.musculus_mRNA_(3C10)_for_IgA_V- D-J-heavy_chain | 1.5 | 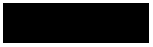 | 1.1 | 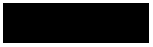 |
| AA050168_proteasome_(prosome,_macropain)_sub- | −1.1 | −1.2 | 1.1 | −1.2 |
| unit,_beta_type_10 | | | | |
| AI451862_RIKEN_cDNA_0910001A18_gene | −2.4 | −1.3 | −1.1 | −1.3 |
| AI386046_trypsin_4 | −2.5 | −1.1 | −1.3 | −1.2 |
| AA386807_RIKEN_cDNA_2310002A12_gene | 1.1 | 1.0 | 1.1 | −1.1 |
| AA822485_proteosome_(prosome,_macropain)_sub- | −1.1 | 1.1 | −1.0 | −1.3 |
| unit,_beta_type_9_(large_multifunctional_protease_2) | | | | |
| AA880322_calponin_1 | −1.5 | −1.2 | −1.4 | −1.0 |
| AA212405_dihydrofolate_reductase | −1.2 | 1.1 | −1.1 | −1.7 |
| AI529513_ESTs,_Moderately_similar_to_F26L_6PF-2-K/ | −1.1 | 1.4 | −1.3 | −1.3 |
| FRU-2,6-P2ASE_LIVE_ISOZYME_[M.musculus] | | | | |
| AA762277_lympocyte_antigen_6_complex,_locus_C | 1.1 | 1.1 | −1.1 | −1.1 |
| AI048040_claudin_4 | −1.2 | −1.3 | −1.2 | −1.2 |
| AI326566_histocompatibility_2,_class_II_antigen_E_alpha | −1.6 | 1.1 | −1.1 | −1.4 |
| W11170_small_inducible_cytokine_A21a_(leucine) | −1.1 | −1.1 | −1.3 | 1.0 |
| AA967824_arginine_vasopressin | −1.3 | 1.4 | −1.2 | −2.3 |
| W08321_inhibitor_of_DNA_binding_1 | −1.0 | 1.0 | −1.0 | 1.2 |
| AI157238_histocompatibility_2,_class_II,_locus_Mb1 | −1.4 | −1.1 | 1.1 | −1.2 |
| AA272836_aquaporin_8 | 1.0 | 1.2 | −2.4 | 1.3 |
| AW496194_immuno- | −1.0 | 1.4 | −1.0 | −2.7 |
| globulin_kappa_chain_variable_20_(V20_family) | | | | |
| AA792785_histocompatibility_2,_class_II,_antigen_E_beta | −1.7 | 1.1 | 1.0 | −1.1 |
| AI428626_ESTs | −1.1 | −1.3 | 1.0 | −1.2 |
| AA871265_Public_domain_EST | 1.2 | 1.3 | 1.4 | −1.7 |
| AA152940_immunoglobulin_kappa_chain_variable_28_(V28) | −1.3 | 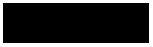 | −1.1 | −2.5 |
| AA754696_immuno- | −1.2 | −1.0 | −1.1 | −1.4 |
| globulin_heavy_chain_6_(heavy_chain_of_IgM) | | | | |
| AA790398_ribonuclease_1,_pancreatic | −4.1 | −1.1 | −1.2 | −1.1 |
| AA072834_RIKEN_cDNA_9030418M05_gene | −1.1 | 1.1 | 1.1 | 1.3 |
| W17930_small_inducible_cytokine_A21a_(leucine) | −1.2 | −1.2 | −1.5 | −1.2 |
| AA242406_CEA-related_cell_adhesion_molecule_1 | −1.1 | −1.0 | 1.2 | 1.1 |
| AI627143_ESTs | −1.2 | −1.1 | −1.1 | −1.0 |
| AA250087_proteosome_(prosome,_macropain)_sub- | −1.1 | 1.2 | −1.0 | −1.2 |
| unit,_beta_type_8_(large_multifunctional_protease_7) | | | | |
| W15809_hemoglobin,_beta_adult_major_chain | −1.0 | 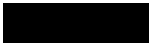 | −1.7 | −1.6 |
| AA759679_Ia-associated_invariant_chain | −1.9 | −1.0 | −1.3 | −1.4 |
| AA790409_sialytranferase_7_(alpha-N-acetylneuraminyl_2,3- | −1.4 | 1.0 | −1.1 | −1.1 |
| betagalactosyl-1,3)-N-acetyl_galactosaminide_alpha-2,6- | | | | |
| sialytransferase)_F | | | | |
| AI605648_small_proline-rich_protein_2A | −1.2 | −1.1 | −1.5 | −1.1 |
| AA821884_amylase_2,_pancreatic | −4.4 | −1.1 | −1.1 | −1.2 |
| AI386257_calbindin-D9K | −1.1 | −1.2 | −1.2 | 1.4 |
| AA066763_hemoglobin,_beta_adult_major_chain | −1.3 | 1.5 | −1.8 | −2.0 |
| AA106071_hemoglobin,_beta_adult_major_chain | −1.3 | 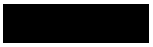 | −1.7 | −2.0 |
| AA199011_histocompatibility_2,_class_II_antigen_A,_alpha | −1.9 | 1.3 | −1.0 | −1.2 |
| AA497618_crp-ductin | −1.3 | −1.2 | 1.5 | 1.2 |
| AA717025_Mus_musculus_10_day_old_male_pan- | −2.9 | 1.2 | −1.1 | −1.4 |
| creas_cDNA,_RIKEN_full-length_enriched_li- | | | | |
| brary,_clone:1810009A17,_full_insert_sequence | | | | |

INTESTINE

| 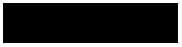 | spl | panc | liv | stom |
|---|---|---|---|---|
| Description | PL-4B_BDEnor | PL-2B_BDEnor | PL-6B_BDEnor | PL-8B_BDEnor |
| 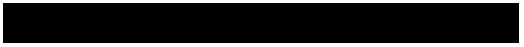 | 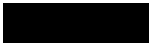 | 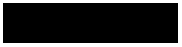 | −1.9 | 1.2 |
| 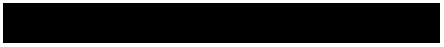 | 1.5 | −1.2 | 1.3 | −1.1 |
| 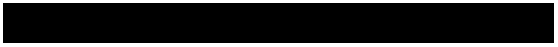 | 1.5 | 1.05393+ | 1.4 | 1.1 |
| 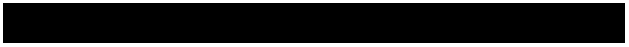 | −1.2 | −1.3 | 1.1 | −1.1 |
| 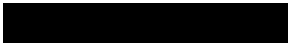 | −1.1 | 1.06509+ | 1.1 | 1.2 |
| 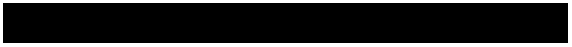 | −1.1 | −1.1 | 1.1 | −1.0 |
| 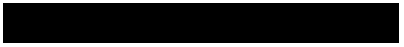 | 1.3 | −1.3 | −1.4 | 1.3 |
| 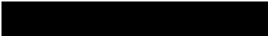 | −1.1 | −1 .0 | −1.2 | 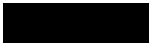 |
| 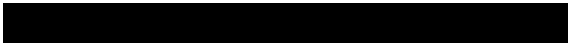 | 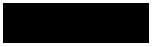 | 1.3 | 1.1 | −1.4 |
| 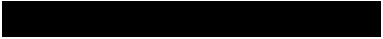 | −1.1 | −1.4 | 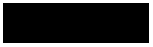 | 1.3 |
| 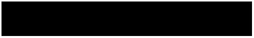 | 1.0 | 1.0 | −1.3 | 1.2 |
| 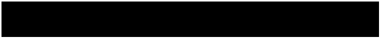 | 1.4 | −1.1 | 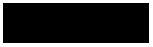 | 1.0 |
| 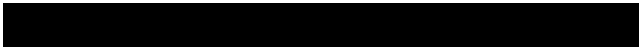 | 1.5 | 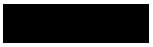 | −1.4 | 1.1 |
| 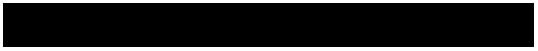 | 1.1 | 1.1 | 1.4 | 1.2 |
| 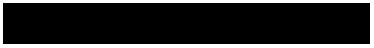 | 1.0 | 1.1 | 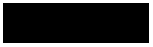 | 1.2 |
| 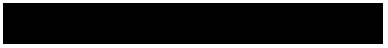 | −1.9 | −1.5 | 1.1 | −2.4 |
| AA871663_defensin_related_cryptdin,_related_sequence_7 | 1.2 | −1.4 | −1.6 | 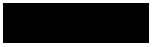 |
| AA793433_glutamyl_aminopeptidase | 1.3 | 1.1 | 1.1 | 1.3 |
| AA869166_defensin_related_cryptdin,_related_sequence_10 | −1.0 | −1.1 | −1.7 | 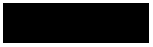 |
| AI324398_glycosylphosphatidylinositol_specific_phospholipase_D1 | 1.1 | −1.3 | 1.2 | −3.4 |
| AA066225_selenoprotein_P,_plasma,_1 | 1.2 | 1.4 | 1.2 | −1.7 |
| AA024217_Public_domain_EST | −2.5 | 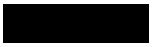 | 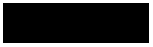 | 1.2 |
| AA882450_RIKEN_cDNA_1200011D11_gene | −1.0 | 1.1 | −1.1 | −1.1 |
| AI385503_cysteine_rich_intestinal_protein | 1.3 | 1.1 | −1.0 | −1.6 |
| AA162211_guanine_nucleotide_binding_protein,_alpha_11 | −1.0 | 1.0 | −1.0 | 1.3 |
| AA415344_histone_deacetylase_7 | 1.2 | 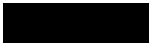 | 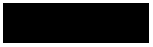 | −1.2 |
| AA239419_P_glycoprotein_2 | 1.0 | −1.2 | −1.0 | −1.1 |
| AA109528_ESTs,_Weakly,_simi- | −1.1 | −1.1 | −1.0 | −1.1 |
| lar_to_CA1C_MOUSE_COLLAGEN_AL- | | | | |
| PHA_1(XII)_CHAIN_PRECURSOR_[M.musculus] | | | | |
| AA770902_actin,_alpha_1,_skeletal_muscle | −1.0 | −1.1 | −1.1 | −1.1 |
| AA647336_cytochrome_P450,_steroid_inducible_3a11 | 1.5 | −1.4 | 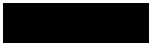 | 1.1 |
| AA386758_ESTs | −1.3 | −1.3 | 1.0 | −1.3 |
| AA871641_defensin_related_cryptdin,_related_sequence_2 | −1.0 | 1.3 | −1.4 | 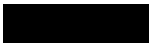 |
| AA871410_defensin_related_cryptdin_5 | −1.5 | −1.1 | −1.0 | 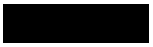 |
| AA2102337_Mus_musculus,_clone_MGC:6377,_mRNA,_com- | −1.1 | 1.0 | 1.0 | −1.0 |
| plete_cds | | | | |
| AI552087_IQ_motif_containing_GTPase_activating_protein_1 | −1.3 | −1.1 | 1.3 | 1.1 |
| AA166427_Mus_musculus_germline_immunoglobulin_gam- | −1.3 | 1.0 | −1.0 | 1.1 |
| ma_constant_region_(IgG3)_mRNA | | | | |
| AA756136_actin,_gamma_2,_smooth_muscle,_enteric | 1.1 | −1.2 | 1.1 | −1.1 |
| AA239727_ATP-binding_cassette, sub- | 1.0 | −1.1 | −1.1 | −1.0 |
| family_B_(MDR/TAP),_member_1 | | | | |
| AA469630_choline_kinase | 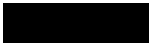 | 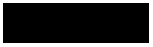 | −1.4 | 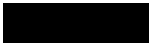 |
| AI594147_beta-2_microglobulin | 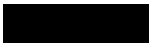 | 1.1 | 1.2 | −2.1 |
| AA684403_ESTs | −1.4 | 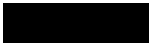 | −2.3 | −2.0 |
| AA869219_intelectin | −1.0 | −1.3 | −1.1 | 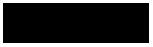 |
| AA073925_ESTs | 1.3 | 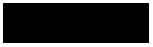 | −1.4 | 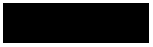 |
| W54384_glutathione_s-transferase,_mu_2 | 1.3 | 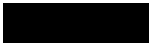 | −1.0 | −1.5 |
| AA013726_cathepsin_J | −1.0 | −1.0 | −1.2 | 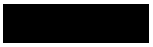 |
| AI549624_RIKEN_cDNA_0610041E09_gene | 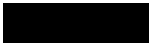 | 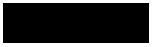 | −1.3 | 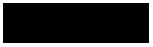 |
| AI552688_ESTs,_Weakly_simi- | −1.2 | 1.2 | 1.1 | 1.1 |
| lar_to_MIA_MOUSE_MELANOMA_DERIVED_GROWTH_REG- | | | | |
| ULATORY_PROTEIN_PRUCURSOR_[M.musculus] | | | | |
| AA450725_membrane_metallo_endopeptidase | 1.3 | −1.3 | 1.1 | −1.1 |
| AA870247_mitogen_regulated_protein,_proliferin_3 | −1.0 | 1.0 | −1.3 | 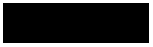 |
| AI892437_transglutaminase_2,_C_polypeptide | −1.1 | −1.2 | 1.0 | 1.3 |
| AI036411_angiotensin_converting_enzyme | −1.1 | −1.0 | 1.2 | 1 .4 |
| AA619767_ornithine_decarboxylase_antizyme | 1.3 | 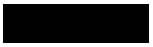 | −1.0 | −1.6 |
| AA106623_hexosaminidase_B | 1.1 | −1.0 | 1.3 | 1.1 |
| A14224_N-myc_downstream_regulated_1 | 1.1 | 1.0 | 1.0 | 1.1 |
| AA624460_actin,_alpha_2,_smooth_muscle,_aorta | −1.0 | 1.2 | −1.3 | −1.1 |
| W36474_metallothionein_2 | −1.2 | −2.2 | 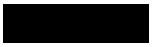 | 1.2 |
| AA821963_aspartyl_aminopeptidase | 1.0 | 1.0 | 1.1 | −1.2 |
| W13521_cytochrome_P450,_2j6 | 1.4 | −1.0 | 1.1 | −1.4 |
| AA413490_transferin_receptor | 1.4 | −1.3 | 1.2 | −1.0 |
| AI594093_Mus_musculus_putative_CAMP_pro- | −1.1 | −1.3 | 1.1 | 1.4 |
| tein_(Camp)_mRNA,_complete_cds | | | | |
| AA028420_small_proline-rich_1A | 1.3 | −1.7 | −1.4 | −1.5 |
| AA245987_ESTs | −1.01124+ | −1.30375+ | 1.1 | 1.29885+ |
| AI390129_proteasome_(prosome,_macropain)_sub- | 1.1 | 1.0 | 1.3 | −1.4 |
| unit,_alpha_type_2 | | | | |
| AI156982_nuclear_receptor_subfamily_5,_group_A,_member_2 | −1.3 | 1.2 | −1.1 | −1.1 |
| AA546964_ESTs,_Weakly_simi- | 1.0 | −1.4 | −1.1 | −1.1 |
| lar_to_TLM_MOUSE_TLM_PROTEIN_[M.musculus] | | | | |
| AI605616_ESTs | −1.2 | −1.1 | 1.3 | −1.5 |
| AA561934_ESTs | −1.5 | −1.5 | −1.4 | 1.1 |
| AI157093_RIKEN_cDNA_2700094F01_gene | −1.0 | −1.6 | −1.0 | −1.1 |
| AA794319_ESTs | −1.1 | −1.1 | 1.3 | −1.7 |
| AA433804_ESTs | −1.1 | −1.1 | −1.1 | −1.2 |
| AA754696_immuno- globulin_heavy_chain_6_(heavy_chain_of_IgM) | 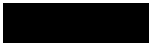 | −1.5 | 1.0 | −1.2 |
| W09198_calcium_binding_protein_A6_(calcyclin) | −1.2 | −1.2 | −1.2 | −1.6 |
| AA967824_arginine_vasopressin | 1.4 | −1.6 | −1.2 | −1.1 |
| AW496194_immunoglobulin_kappa_chain_variable_20_(V20_family) | 1.4 | −1.2 | −1.4 | −1.1 |
| AA647876_ESTs | −1.3 | −1.3 | −1.0 | 1.3 |
| AA790455_RIKEN_cDNA_3110001I20_gene | 1.1 | −1.1 | 1.2 | −1.1 |
| AA068562_RIKEN_cDNA_2810417H13_gene | −1.0 | −1.5 | −1.1 | −1.1 |
| AI627053_ESTs | −1.3 | −1.1 | −1.1 | 1.1 |
| AI607043_ESTs,_Weakly_similar_to_S12207_hypo- | 1.2 | −1.2 | −1.1 | −1.2 |
| thethical_protein_[M/musculus] | | | | |
| AI536309_RIKEN_cDNA_2210410L06_gene | −1.8 | −1.8 | −1.4 | −2.1 |
| AA166386_transducer_of_ErbB-2.1 | −1.0 | −1.2 | 1 .3 | −2.0 |
| AA414465_ESTs | −1.2 | −1.3 | −1.0 | 1.0 |
| AA529377_heat_shock_protein,_86_kDa_1 | 1.4 | 1.0 | −1.3 | −1.9 |
| AA562129_ESTs,_Weakly_similar_to_T23699_hypo- | −1.0 | −1.4 | 1.1 | −1.2 |
| thetical_protein_M04B2.4_-_Caenorhabditis_elegans_[C.elegans] | | | | |
| AI428582_Public_domain_EST | 1.0 | −1.5 | −1.1 | −1.3 |
| AA738642_proteasome_(prosome,_macropain)_sub- | 1.2 | −1.0 | 1.4 | −1.3 |
| unit,_alpha_type_3 | | | | |
| AI596504_t-complex_testis_expressed_1 | 1.1 | −1.6 | −1.0 | 1.3 |
| AI036487_ESTs | −1.0 | 1.1 | −1.1 | −1.3 |
| AI386181_gamma-glutamyl_carboxylase | −1.1 | −1.3 | −1.0 | −1.2 |
| AA422743_ESTs,_Weakly_similar_to_AF091622_1_PHD_fin- | −1.5 | −1.3 | −1.2 | 1.1 |
| ger_protein_3_[H.sapiens] | | | | |
| AA289639_ESTs | −1.2 | −1.2 | −1.1 | 1.1 |
| W84257_interferon_(alpha_and_beta)_receptor | −1.0 | −1.4 | −1.1 | −1.2 |
| AI429064_ESTs,_Weakly_similar_to_S12207_hypo- | 1.0 | −1.5 | 1.0 | −1.1 |
| thetical_protein_[M.musculus] | | | | |
| AA197547__RIKEN_cDNA_2310031L18_gene | −1.1 | −1.4 | −1.1 | −1.1 |
| AA563376_DNA_segment,_Chr_8,_ERATO_Doi_233,_expressed | −1.2 | −1.1 | 1.1 | −1.0 |
| AA414730_NCK-associated_protein_1 | 1.2 | −1.1 | 1.0 | −1.8 |
| AA276909_ESTs,_Weakly_simi- | −1.3 | −1.8 | −1.2 | −1.1 |
| lar_to_GRBA_MOUSE_GROWTH_FACTOR_RECEPTOR- | | | | |
| BOUND_PROTEIN_10_[M/musculus] | | | | |
| AA638300_ESTs,_Moderately_similar_to_S12207_hypo- | 1.0 | −1.2 | −1.2 | −1.1 |
| thetical_protein_[M.musculus] | | | | |
| W34620_kallikrein_26 | 1.2 | 1.1 | −1.1 | −1.4 |
| AI592338_ESTs | 1.08777+ | −1.50505+ | −1.48731+ | −1.6 |
| AA727388_ESTs,_Moderately_similar_to_S12207_hypo- | −1.0 | −1.1 | 1.0 | −1.1 |
| thetical_protein_[M.musculus] | | | | |
| AA437755_thymus_expressed_acidic_protein | −1.3 | −1.5 | −1.0 | −1.1 |
| AA981354_cyclin_T1 | −1.4 | −1.2 | −1.2 | −1.6 |
| AI465828_ESTs | −1.2 | −1.4 | −1.3 | −1.2 |
| AA467531_double_C2,_gamma | −1.2 | −1.3 | −1.3 | −1.4 |
| AA856528_ESTs,_Highly_similar_to_S12207_hypo- | 1.0 | −1.1 | 1.0 | −1.3 |
| thetical_protein_[M.musculus] | | | | |
| [symbol] | 1.2 | −1.142323+ | −1.16278+ | −1.4 |
| AA673741_DNA_segment,_Chr_12,_ERATO_Doi_604,_expressed | 1.3 | −1.0 | −1.1 | −1.4 |
| AA619869_RIKEN_cDNA_5730534O06_gene | 1.1 | −1.0 | 1.1 | −1.2 |
| AI390319_ESTs,_Weakly_similar_to_apolipoprotein_F_[H.sapiens] | −1.1 | −1.5 | 1.2 | −1.1 |
| AI614000_ESTs | −1.1 | −1.2 | 1.0 | −1.1 |
| AA794176_ESTs,_Moderately_similar_to_SPA- | −1.0 | −2.0 | −1.2 | −1.2 |
| 1_like_protein_p1294_[R.norvegicus] | | | | |
| AI156378_ESTs | −1.1 | −1.7 | −1.2 | −1.1 |
| AA656612_RIKEN_cDNA_4631416G20_gene | −1.1 | −1.3 | 1.1 | −1.1 |
| AA177238_ESTs | −1.2 | −1.2 | 1.0 | 1.1 |
| AA023331_adenosine_deaminase | 1.1 | 1.1 | −1.1 | 1.1 |
| AA415510_ESTs | −1.0 | −1.4 | −1.1 | −1.3 |
| AI447943_EST | −1.1 | −1.3 | −1.2 | −1.2 |
| AA623448_ESTs,_Weakly_similar_to_NED5_MOUSE_NEDD- | 1.3 | −1.2 | −1.0 | −1.1 |
| 4_PROTEIN_[M.musculus] | | | | |
| AI120315_splicing_factor,_arginine/serine- rich_5_(SRp40,_HRS) | 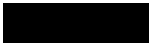 | −1.4 | 1.2 | −1.1 |
| AA509560_ESTs | −1.1 | −1.3 | 1.2 | −1.1 |
| AA692960_ESTs,_Weakly_similar_to_AF089896_1_topoisomerase- | 1.1 | −1.5 | 1.0 | −1.1 |
| related_function_protein_[H.sapiens] | | | | |
| AA469667_tumor_rejection_antigen_gp96 | 1.3 | 1.4 | −1.5 | −1.9 |
| AA982569_ESTs | −1.1 | −1.5 | −1.1 | −1.2 |
| W14153_ESTs,_Moderately_similar_to_S12207_hypo- | −1.0 | −1.1 | 1.1 | −1.1 |
| thetical_protein_[M.musculus] | | | | |
| AI596800_ESTs | −1.4 | −1.3 | −1.1 | −1.3 |
| AA638908_ESTs | 1.0 | −1.6 | −1.0 | −1.1 |
| AI605657_serine/arginine-rich_protein_specific_kinase_2 | −1.1 | −1.0 | 1.0 | 1.1 |
| AA795130_ESTs,_Moderately_similar_to_S12207_hypo- | 1.0 | −1.3 | −1.0 | −1.1 |
| thetical_protein_[M.musculus] | | | | |
| AA146478_M.musculus_mRNA_(3C10)_for_IgA_V-D-J- heavy_chain | −1.3 | 1.0 | −1.6 | 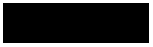 |
| AA397221_ESTs,_Weakly_similar_to_NED4_MOUSE_NEDD- | −1.1 | −1.2 | −1.0 | −1.0 |
| 4_PROTEIN_[M.musculus] | | | | |
| AA717269_ESTs,_Weakly_similar_to_T46456_hypo- | 1.1 | −1.7 | 1.0 | −1.1 |
| thetical_protein_DKFZp4346132.1_[H.sapiens] | | | | |
| AI666289_ESTs | −1.1 | −1.4 | −1.0 | −1.1 |
| AI552745_Mus_musculus,_clone_MGC:6888,_mRNA,_com- | 1.1 | 1.1 | 1.1 | −2.0 |
| plete_cds | | | | |
| W406908_anterior_gradient_2_(Xenopus_laevis) | 1.4 | 1.1 | −1.4 | −1.7 |
| AI892534_ribosomal_protein_S24 | 1.3 | 1.1 | 1.3 | −2.1 |
| AA450438_Mus_musculus_mRNA_for_erythroid_dif- | −1.0 | −1.0 | −1.2 | −1.5 |
| ferentiation_regulator,_partial | | | | |
| AA619114_solute_carrier_family_11_(proton- | 1.1 | 1.0 | 1.0 | −1.2 |
| coupled_divalent_metal_ion_transporters),_member_2 | | | | |
| AA608285_ubiquitin_fusion_degradation_1_like | −1.2 | −1.4 | −1.0 | −1.3 |
| AA549540_ESTs | 1.1 | −1.4 | −1.0 | −1.0 |
| AA066867_RIKEN_cDNA_3010002H13_gene | 1.3 | −1.1 | 1.0 | −1.1 |
| AI608085_ESTs | −1.1 | −1.5 | −1.1 | −1.3 |
| AA474446_ESTs | −1.3 | −1.5 | −1.1 | 1.3 |
| AA444672_RIKEN_cDNA_2510001A17_gene | −1.1 | −1.0 | 1.1 | −2.3 |
| AA473972_heterogeneous_nucluar_ribonucleoprotein_H1 | 1.5 | −1.2 | 1.2 | −1.8 |
| AA267106_ESTs | −1.1 | −1.3 | 1.1 | −1.1 |
| AA638929_ESTs,_Moderately_similar_to_S12207_hypo- | 1.0 | −1.3 | −1.0 | −1.1 |
| thetical_protein_[M.musculus] | | | | |
| AI391001_Public_domain_EST | −1.2 | −1.3 | −1.0 | 1.0 |
| AA575047_ESTs | −1.3 | −1.4 | −1.1 | 1.1 |
| AI450530_ESTs | −1.1 | −1.3 1.1 | −1.3 | |
| AA536789_RIKEN_cDNA_1110002B05_gene | 1.2 | 1.0 | 1.5 | −1.3 |
| AA684191_CDC-like_kinase | 1.5 | −2.1 | −1.3 | −1.1 |
| AA549591_ESTs,_Highly_similar_to_CRM1_[H.sapiens] | −1.2 | −1.7 | −1.1 | −1.4 |
| AI536183_Public_domain_EST | 1.0 | −1.1 | 1.0 | 1.1 |
| AA874133_ESTs | −1.2 | −1.6 | 1.1 | −1.2 |
| AA684092_ESTs,_Moderately_similar_to_S12207_hypo- | 1.0 | −1.4 | 1.0 | −1.2 |
| thetical_protein_[M.musculus] | | | | |
| AA672766_Mouse_mRNA_for_TI-227 | −1.5 | −1.3 | −1.0 | −1.1 |
| AA164064_ESTs | −1.1 | −1.7 | −1.1 | −1.0 |
| W15606_dynein,_axon,_heavy_chain_11 | 1.3 | −1.3 | −1.1 | −2.5 |
| AA450914_RIKEN_cDNA_1810005K14_gene | −1.1 | −1.0 | −1.06093** | −1.4 |
| AA896036_ESTs,_Highly_similar_to_GUANINE_NUCLEOTIDE- | −1.2 | −1.2 | −1.1 | 1.0 |
| BINDING_PROTEIN_BETA_SUBUNIT- | | | | |
| LIKE_PROTEIN_12.3_[M.musculus] | | | | |
| AA914844_Public_domain_EST | 1.3 | −1.5 | 1.0 | −1.2 |
| AA059886_protein_phosphatase,_EF_hand_calcium- | 1.1 | −1.6 | −1.1 | −1.1 |
| binding_domain_2 | | | | |
| AA067797_heat_shock_protein_cognate_70 | 1.0 | −1.1 | −1.0 | −2.1 |
| AI530648_ESTs | −1.6 | −1.1 | 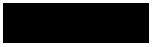 | −1.2 |
| AA619915_RIKEN_cDNA_1110015M06_gene | 1.2 | −1.3 | 1.1 | 1.3 |
| AA000282_naked_cuticle_1_homolog_(Drosophila) | −1.0 | −1.3 | −1.0 | 1.0 |
| AA607713_Mus_musculus_10_day_old_male_pan- | 1.1 | 1.5 | −1.2 | −1.5 |
| creas_cDNA,_RIKEN_full- | | | | |
| length_enriched_library,_clone:1810009A17,_full_insert_sequence | | | | |
| AA152940_immunoglobulin_kappa_chain_variable_28_(V28) | 1.3 | −1.6 | −1.5 | −1.0 |
| AI640116_ESTs | −1.6 | −1.02516+ | 1.3 | −1.1 |
| AA260035_ESTs | −1.1 | −1.3 | 1.0 | 1.0 |
| AA444576_heat_shock_protein,_60_kDa | 1.1 | −1.1 | 1.4 | −1.8 |
| AI451754_ESTs | 1.0 | −1.3 | −1.0 | 1.1 |
| AI019837_Iaminin,_beta_3 | 1.1 | 1.4 | −1.1 | −1.5 |
| AA066404_topoisomerase_(DNA)_II_beta | 1.1 | −1.2 | 1.2 | −2.2 |
| AA880295_immunoglobulin_joining_chain | 1.2 | −1.5 | −1.4 | 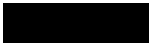 |
| AA200306_ESTs | −1.5 | −1.3 | −1.5 | −1.0 |
| AI645264_ESTs,_Weakly_similar_to_S29170_an- | −1.1 | −1.1 | −1.1 | −1.2 |
| nexin_VII_-_mouse_[M.musculus] | | | | |
| W56995_hemoglobin_X,_alpha- | 1.1 | −1.6 | −1.3 | −1.2 |
| like_embryonic_chain_in_Hba_complex | | | | |
| AA288278_ESTs | −1.3 | −1.5 | −1.3 | −1.3 |
| AI463733_ESTs,_Moderately_similar_to_S12207_hypo- | 1.0 | −1.3 | −1.0 | −1.2 |
| thetical_protein_-_mouse_[M.musculus] | | | | |
| AA152636_integrin_alpha_4 | 1.2 | −2.2 | −1.3 | 1.1 |
| AA138854_ESTs,_Weakly_similar_to_lyso- | −1.4 | −1.6 | −1.1 | −1.0 |
| phospholipase_I_[M.musculus] | | | | |
| AI117296_Paneth_cell_enhanced_expression | 1.3 | −1.2 | 1.1 | 1.1 |
| AI592114_molybdenum_cofactor_synthesis_2 | 1.1 | −1.7 | −1.0 | 1.2 |
| AA435317_ESTs | −1.2 | −1.7 | 1.1 | −1.0 |
| AA105092_Bruton_agammaglobulinemia_tyrosine_kinase | −1.4 | 1.0 | 1.2 | −1.4 |
| [symbol] regulated_protein,_78kD) | 1.1 | 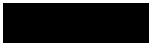 | −2.4 | −2.2 |
| AA466153_ESTs | 1.0 | −1.11378+ | 1.02861+ | 1.2 |
| AA718696_ESTs | −1.6 | −1.3 | −1.2 | −1.3 |
| AA212405_dihydrofolate_reductase | −1.2 | −1.6 | −1.2 | −1.0 |
| AA727065_ESTs | 1.1 | −1.2 | 1.2 | −2.1 |
| AI464865_ESTs | −1.3 | 1.1 | 1.0 | 1.3 |
| AI627143_ESTs | −1.0 | −1.1 | 1.1 | −1.3 |
| AA065764_AXL_receptor_tyrosine_kinase | −1.3 | 1.0 | 1.1 | 1.0 |
| AI390861_retinoic_acid_induced_1 | −1.5 | 1.0 | −1.3 | −1.2 |
| AA619964_nuclear_receptor_coactivator_4 | 1.4 | −1.3 | 1.4 | −1.7 |
| AA498574_kallikrein_6 | 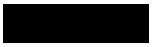 | 1.5 | −1.2 | −1.4 |
| AI596527_ribosomal_protein_L26 | 1.2 | 1.1 | 1.3 | −1.8 |
| AA521693_Public_domain_EST | −1.6 | −1.3 | −1.6 | −1.5 |
| AA210505_Public_domain_EST | 1.4 | −1.5 | 1.2 | −2.1 |
| AA242360_cytochrome_P450,_1a2,_aro- | 1.3 | −1.5 | 1.4 | −1.1 |
| matic_compound_inducible | | | | |
| AI536352_G_protein- | −1.3 | −1.7 | −1.4 | −2.1 |
| coupled_receptor,_family_C,_group_5,_member_B | | | | |
| AA693081_alkaline_phosphatase_5 | 1.14857+ | −1.15714+ | 1.38347+ | −1.05793+ |
| AA105866_glytathione_S-transferase,_alpha_4 | 1.1 | 1.0 | 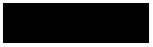 | −2.4 |
| AI425738_ESTs,_Weakly_simi- | −1.3 | −1.6 | −1.3 | −1.1 |
| lar_to_ACRO_HUMAN_ACROSIN_PRECURSO_[H.sapiens] | | | | |
| AA790247_RIKEN_cDNA_2210407P13_gene | 1.01667+ | −1.39059+ | −1.0 | 1.2 |
| AI614443_3′-phosphoadenosine_5′-phosphosulfate_synthase_2 | 1.2 | −1.2 | 1.5 | 1.2 |
| AA839435_cathepsin_E | −1.0 | −1.5 | 1.0 | −1.8 |
| AI605648_small_proline-rich_protein_2A | −1.0 | −1.1 | −1.4 | −1.3 |
| AI327450_phospholipase_A2,_group_IB,_pancreas | 1.3 | 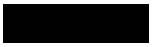 | 1.1 | −1.8 |
| AA674409_pancreatic_lipase_related_protein_1 | 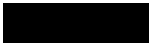 | 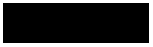 | −1.4 | −1.7 |
| AA497618_crp-ductin | 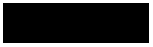 | 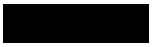 | −1.1 | −1.6 |
| AA107101_prostate_stem_cell_antigen | −1.1 | −1.4 | −1.3 | −2.1 |
| W12985_annexin_A10 | 1.5 | −1.7 | −1.1 | −2.3 |
| AI876997_carboxyl_ester_lipase | 1.5 | 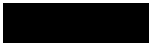 | −1.4 | −2.3 |
| AA982254_RIKEN_cDNA_2210010C04_gene | 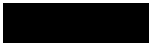 | 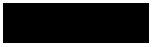 −1.3 | −2.0 | |
| AA237793_rat_regenerating_islet-derived,_mouse_homolog_1 | 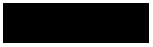 | 1.4 | −1.3 | −3.9 |
| AA675111_RIKEN_cDNA_1810007A24_gene | 1.4 | 1.3 | −1.0 | −2.1 |
| AI451862_RIKEN_cDNA_0910001A18_gene | 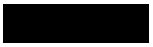 | 1.3 | −1.1 | −2.5 |
| AI386046_trypsin_4 | 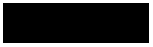 | 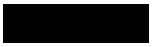 | 1.1 | −1.8 |
| AA790398_ribonuclease_1,_pancreatic | 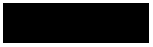 | 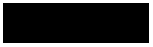 | −1.2 | −5.8 |
| AA675084_elastase_2 | 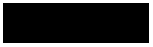 | 1.4 | −1.2 | −2.7 |
| AA717025_Mus_musculus_10_day_old_male_pan- creas_cDNA,_RIKEN_full- length_enriched_library,_clone:1810009A17,_full_insert_sequence | 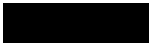 | −1.1 | −1.2 | −2.7 |
| AA821884_amylase_2,_pancreatic | 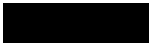 | 1.1 | −1.2 | −2.8 |
| AI386257_calbindin-D9k | −2.2 | −1.3 | 1.2 | 1.5 |
| AI894032_trefoil_factor_2_(spasmolytic_protein_1) | −1.1 | 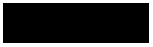 | −1.2 | −2.0 |

INTESTINE

| 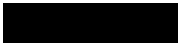 | 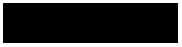 | col | br | lung |
|---|---|---|---|---|
| Description | PL-3B_BDEnor | PL-5B_BDEnor | PL-1B_BDEnor | PL-7B_BDEnor |
| 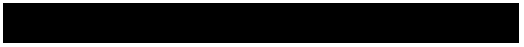 | 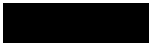 | −2.4 | −1.1 | −1.3 |
| 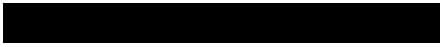 | 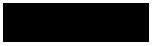 | 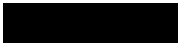 | −1.1 | −2.2 |
| 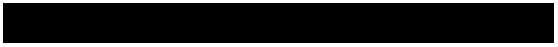 | 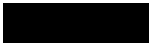 | −1.4304˜ | 1.2 | 1.1 |
| 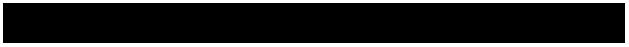 | 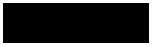 | 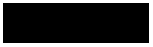 | −1.5 | −1.9 |
| 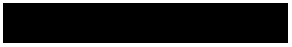 | 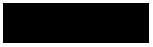 | 1.27917+˜ | −1.2 | 1.0 |
| 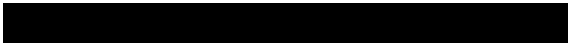 | 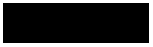 | 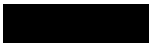 | 1.0 | 1.0 |
| 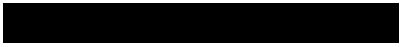 | 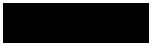 | 1.1 | −1.5 | −1.3 |
| 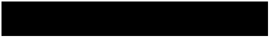 | 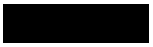 | 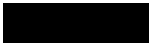 | −1.9 | −1.3 |
| 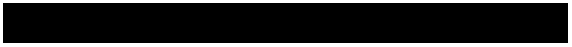 | 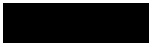 | 1.4 | −1.1 | −1.1 |
| 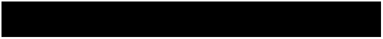 | 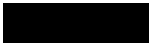 | 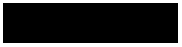 | −1.3 | 1.1 |
| 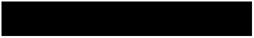 | 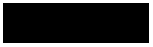 | −2.1 | −1.5 | −1.1 |
| 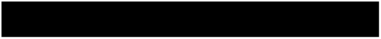 | 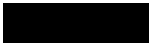 | 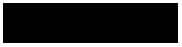 | 1.0 | 1.1 |
| 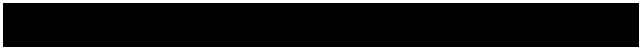 | 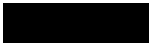 | −1.0 | −1.8 | 1.2 |
| 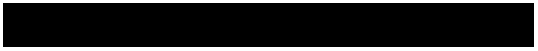 | 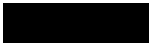 | 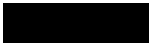 | 1.3 | 1.2 |
| 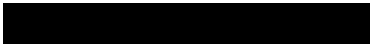 | 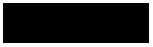 | 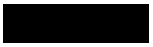 | 1.2 | −1.6 |
| 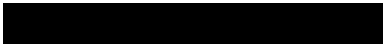 | 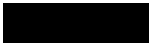 | 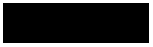 | −1.9 | −9.0 |
| AA871663_defensin_related_cryptdin,_related_sequence_7 | 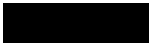 | 1.3 | −1.2 | −1.5 |
| AA793433_glutamyl_aminopeptidase | 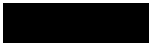 | −1.0 | 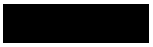 | 1.1 |
| AA869166_defensin_related_cryptdin,_related_sequence_10 | 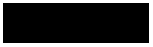 | 1.5 | 1.2 | 1.2 |
| AI324398_glycosylphosphatidylinositol_specific_phospholipase_D1 | 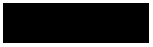 | −1.5 | 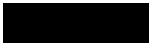 | −1.2 |
| AA066225_selenoprotein_P,_plasma,_1 | 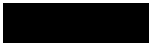 | 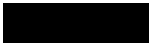 | 1.5 | −1.2 |
| AA024217_Public_domain_EST | 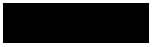 | 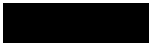 | −1.0 | −4.2 |
| AA882450_RIKEN_cDNA_1200011D11_gene | 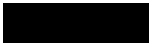 | 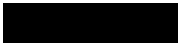 | −1.4 | −1.0 |
| AI385503_cysteine_rich_intestinal_protein | 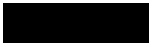 | 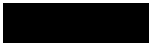 | −1.3 | −1.1 |
| AA162211_guanine_nucleotide_binding_protein,_alpha_11 | 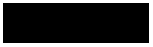 | 1.2 | 1.2 | 1.0 |
| AA415344_histone_deacetylase_7 | 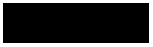 | 1.28947˜ | 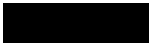 | 1.5 |
| AA239419_P_glycoprotein_2 | 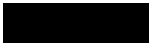 | 1.0 | 1.0 | 1.0 |
| AA109528_ESTs,_Weakly,_simi- lar_to_CA1C_MOUSE_COLLAGEN_AL- PHA_1(XII)_CHAIN_PRECURSOR_[M.musculus] | 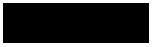 | 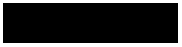 | −1.5 | 1.0 |
| AA770902_actin,_alpha_1,_skeletal_muscle | 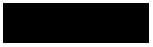 | 1.1 | −1.5 | 1.1 |
| AA647336_cytochrome_P450,_steroid_inducible_3a11 | 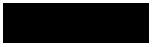 | 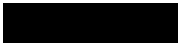 | −1.3 | −8.0 |
| AA386758_ESTs | 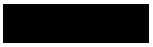 | 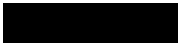 | 1.1 | 1.3 |
| AA871641_defensin_related_cryptdin,_related_sequence_2 | 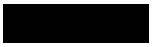 | 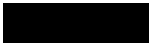 | 1.2 | 1.4 |
| AA871410_defensin_related_cryptdin_5 | 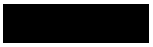 | 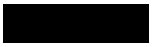 | −1.0 | −1.2 |
| AA2102337_Mus_musculus,_clone_MGC:6377,_mRNA,_com- plete_cds | 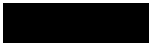 | 1.5 | 1.3 | 1.3 |
| AI552087_IQ_motif_containing_GTPase_activating_protein_1 | 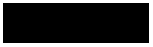 | 1.2 | −1.2 | 1.4 |
| AA166427_Mus_musculus_germline_immunoglobulin_gam- ma_constant_region_(IgG3)_mRNA | 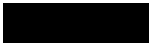 | −1.2153+˜ | 1.3 | 1.0 |
| AA756136_actin,_gamma_2,_smooth_muscle,_enteric | 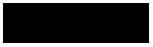 | −1.3 | 1.0 | 1.2 |
| AA239727_ATP-binding_cassette, sub- family_B_(MDR/TAP),_member_1 | 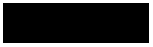 | 1.4 | 1.2 | −1.1 |
| AA469630_choline_kinase | 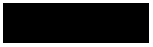 | 1.31548+˜ | 1.3 | 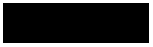 |
| AI594147_beta-2_microglobulin | 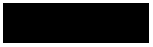 | −1.8 | −1.0 | −1.1 |
| AA684403_ESTs | 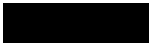 | −2.4 | 1.5 | −1.0 |
| AA869219_intelectin | 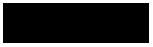 | 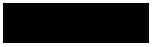 | −1.9 | 1.3 |
| AA073925_ESTs | 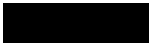 | −1.27077˜ | 1.1 | 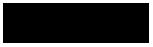 |
| W54384_glutathione_s-transferase,_mu_2 | 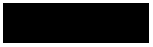 | 1.4 | −1.4 | 1.1 |
| AA013726_cathepsin_J | 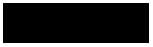 | −1.4 | −1.2 | 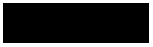 |
| AI549624_RIKEN_cDNA_0610041E09_gene | 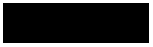 | −1.01549+˜ | 1.1 | 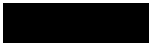 |
| AI552688_ESTs,_Weakly_simi- lar_to_MIA_MOUSE_MELANOMA_DERIVED_GROWTH_REG- ULATORY_PROTEIN_PRUCURSOR_[M.musculus] | 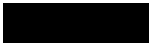 | 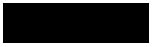 | 1.1 | −1.2 |
| AA450725_membrane_metallo_endopeptidase | 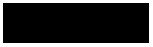 | 1.48772˜ | −1.2 | 1.2 |
| AA870247_mitogen_regulated_protein,_proliferin_3 | 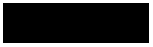 | −1.0 | −1.8 | 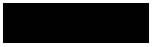 |
| AI892437_transglutaminase_2,_C_polypeptide | 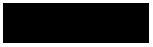 | −1.3 | −1.0 | 1.1 |
| AI036411_angiotensin_converting_enzyme | 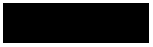 | 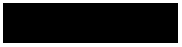 | 1.1 | −1.2 |
| AA619767_ornithine_decarboxylase_antizyme | 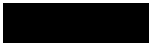 | −1.0 | −1.2 | −1.2 |
| AA106623_hexosaminidase_B | 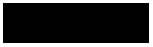 | 1.4 1.3 | −1.1 | |
| A14224_N-myc_downstream_regulated_1 | 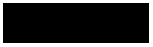 | −1.0 | 1.2 | 1.1 |
| AA624460_actin,_alpha_2,_smooth_muscle,_aorta | 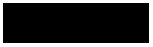 | −1.3 | −1.6 | 1.1 |
| W36474_metallothionein_2 | 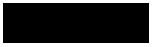 | 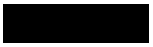 | 1.1 | −1.6 |
| AA821963_aspartyl_aminopeptidase | 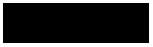 | 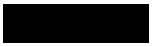 | 1.1 | −1.0 |
| W13521_cytochrome_P450,_2j6 | 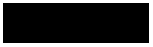 | 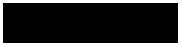 | 1.3 | −1.3 |
| AA413490_transferin_receptor | 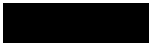 | −1.1 | 1.2 | 1.2 |
| AI594093_Mus_musculus_putative_CAMP_pro- tein_(Camp)_mRNA,_complete_cds | −1.9 | −1.14409+˜ | 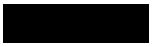 | 1.2 |
| AA028420_small_proline-rich_1A | −1.9 | −1.2 | −1.9 | −1.2 |
| AA245987_ESTs | −1.9 | 1.12119+˜ | −1.46943+ | −1.02333+ |
| AI390129_proteasome_(prosome,_macropain)_sub- | −1.9 | −1.6 | 1.1 | −1.2 |
| unit,_alpha_type_2 | | | | |
| AI156982_nuclear_receptor_subfamily_5,_group_A,_member_2 | −1.9 | −1.40144+*˜ | −1.0 | −1.4 |
| AA546964_ESTs,_Weakly_simi- | −1.9 | 1.1 | 1.4 | 1.2 |
| lar_to_TLM_MOUSE_TLM_PROTEIN_[M.musculus] | | | | |
| AI605616_ESTs | −1.9 | −2.3 | −1.4 | −1.4 |
| AA561934_ESTs | −1.9 | −1.03655+˜ | 1.3 | −1.2 |
| AI157093_RIKEN_cDNA_2700094F01_gene | −1.9 | −1.1 | 1.2 | 1.1 |
| AA794319_ESTs | −1.9 | 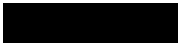 | −1.2 | −1.4 |
| AA433804_ESTs | −1.9 | −1.0 | −1.0 | −1.0 |
| AA754696_immuno- | −1.9 | −3.2 | −1.9 | −1.0 |
| globulin_heavy_chain_6_(heavy_chain_of_IgM) | | | | |
| W09198_calcium_binding_protein_A6_(calcyclin) | −1.9 | −2.4 | 1.1 | 1.1 |
| AA967824_arginine_vasopressin | −1.9 | −2.7 | −2.0 | −1.4 |
| AW496194_immunoglobulin_kappa_chain_variable_20_(V20_family) | −1.9 | −2.9 | 31 2.6 | −1.6 |
| AA647876_ESTs | −1.9 | 1.14107+˜ | −1.0 | −1.2 |
| AA790455_RIKEN_cDNA_3110001I20_gene | −1.9 | −1.50429˜ | 1.2 | −1.1 |
| AA068562_RIKEN_cDNA_2810417H13_gene | −1.9 | −1.0 | −1.0 | −1.2 |
| AI627053_ESTs | −1.9 | −1.03772+˜ | 1.3 | −1.0 |
| AI607043_ESTs,_Weakly_similar_to_S12207_hypo- | −1.9 | 1.1 | 1.0 | 1.1 |
| thethical_protein_[M/musculus] | | | | |
| AI536309_RIKEN_cDNA_2210410L06_gene | −1.9 | 1.1 | −1.5 | −2.0 |
| AA166386_transducer_of_ErbB-2.1 | −1.9 | −1.1 | 1.1 | −1.2 |
| AA414465_ESTs | −1.9 | 1.2 | 1.0 | −1.0 |
| AA529377_heat_shock_protein,_86_kDa_1 | −1.9 | −1.5 | 1.4 | 1.3 |
| AA562129_ESTs,_Weakly_similar_to_T23699_hypo- | −1.9 | 1.1 | 1.1 | 1.1 |
| thetical_protein_M04B2.4_-_Caenorhabditis_elegans_[C.elegans] | | | | |
| AI428582_Public_domain_EST | −1.9 | 1.2 | 1.3 | 1.2 |
| AA738642_proteasome_(prosome,_macropain)_sub- | −1.9 | −1.5 | 1.1 | −1.1 |
| unit,_alpha_type_3 | | | | |
| AI596504_t-complex_testis_expressed_1 | −1.9 | 1.4 | 1.1 | 1.4 |
| AI036487_ESTs | −1.9 | −1.9 | 1.0 | 1.0 |
| AI386181_gamma-glutamyl_carboxylase | −1.9 | 1 .0 | −1.0 | 1.1 |
| AA422743_ESTs,_Weakly_similar_to_AF091622_1_PHD_fin- | −1.9 | −1.4 | −1.2 | 1.1 |
| ger_protein_3_[H.sapiens] | | | | |
| AA289639_ESTs | −1.9 | −1.27398˜ | 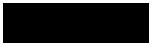 | 1.1 |
| W84257_interferon_(alpha_and_beta)_receptor | −1.9 | 1.1 | −1.1 | 1.1 |
| AI429064_ESTs,_Weakly_similar_to_S12207_hypo- | −1.9 | 1.3 | 1.1 | 1.2 |
| thetical_protein_[M.musculus] | | | | |
| AA197547__RIKEN_cDNA_2310031L18_gene | −1.9 | −1.05207+˜ | 1.1 | −1.0 |
| AA563376_DNA_segment,_Chr_8,_ERATO_Doi_233,_expressed | −1.9 | 1.12124˜ | −1.6 | −1.1 |
| AA414730_NCK-associated_protein_1 | −1.9 | −12. | 1.2 | 1.1 |
| AA276909_ESTs,_Weakly_simi- | −1.9 | −1.29366˜ | −1.3 | 1.2 |
| lar_to_GRBA_MOUSE_GROWTH_FACTOR_RECEPTOR- | | | | |
| BOUND_PROTEIN_10_[M/musculus] | | | | |
| AA638300_ESTs,_Moderately_similar_to_S12207_hypo- | −1.9 | 1.1 | −1.2 | 1.1 |
| thetical_protein_[M.musculus] | | | | |
| W34620_kallikrein_26 | −1.9 | −12. | −1.5 | 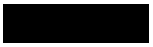 |
| AI592338_ESTs | −1.9 | −1.40845+˜ | −2.12716+ | −1.39471+ |
| AA727388_ESTs,_Moderately_similar_to_S12207_hypo- | −1.9 | 1.0 | −1.1 | −1.0 |
| thetical_protein_[M.musculus] | | | | |
| AA437755_thymus_expressed_acidic_protein | −1.9 | −1.49997+˜ | −1.4 | −1.2 |
| AA981354_cyclin_T1 | −1.9 | −1.2 | −1.4 | −1.1 |
| AI465828_ESTs | −1.9 | −1.24163+˜ | −1.0 | −1.1 |
| AA467531_double_C2,_gamma | −1.9 | −1.24163+˜ | −1.0 | −1.1 |
| AA856528_ESTs,_Highly_similar_to_S12207_hypo- | −1.9 | −1.1 | −1.2 | −1.1 |
| thetical_protein_[M.musculus] | | | | |
| [symbol] | −1.9 | −1.16394˜ | −1.9 | 1.2 |
| AA673741_DNA_segment,_Chr_12,_ERATO_Doi_604,_expressed | −1.9 | 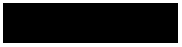 | −1.1 | −1.5 |
| AA619869_RIKEN_cDNA_5730534O06_gene | −1.9 | −1.1 | 1.3 | −1.0 |
| AI390319_ESTs,_Weakly_similar_to_apolipoprotein_F_[H.sapiens] | −1.9 | 1.3 | −1.4 | 1.0 |
| AI614000_ESTs | −2.0 | 1.2 | −1.2 | 1.1 |
| AA794176_ESTs,_Moderately_similar_to_SPA- | −2.0 | 1.2 | 1.2 | 1.3 |
| 1_like_protein_p1294_[R.norvegicus] | | | | |
| AI156378_ESTs | −2.0 | −1.02813˜ | −1.3 | −1.4 |
| AA656612_RIKEN_cDNA_4631416G20_gene | −2.0 | −1.2 | 1.1 | 1.0 |
| AA177238_ESTs | −2.0 | −1.31702+˜ | 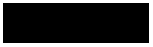 | 1.0 |
| AA023331_adenosine_deaminase | −2.0 | −1.4 | 1.2 | 1.2 |
| AA415510_ESTs | −2.0 | −1.1 | 1.2 | 1.1 |
| AI447943_EST | −2.0 | 1.0 | −1.2 | 1.1 |
| AA623448_ESTs,_Weakly_similar_to_NED5_MOUSE_NEDD- | −2.0 | 1.2 | 1.0 | 1.3 |
| 4_PROTEIN_[M.musculus] | | | | |
| AI120315_splicing_factor,_arginine/serine- | −2.0 | −1.0 | −1.3 | −1.0 |
| rich_5_(SRp40,_HRS) | | | | |
| AA509560_ESTs | −2.0 | 1.0 | 1.2 | 1.1 |
| AA692960_ESTs,_Weakly_similar_to_AF089896_1_topoisomerase- | −2.0 | 1.1 | −1.1 | 1.1 |
| related_function_protein_[H.sapiens] | | | | |
| AA469667_tumor_rejection_antigen_gp96 | −2.0 | −1.4 | 1.2 | −1.1 |
| AA982569_ESTs | −2.0 | 1.0 | 1.12345* | 1.1 |
| W14153_ESTs,_Moderately_similar_to_S12207_hypo- | −2.0 | −1.1 | −1.2 | 1.1 |
| thetical_protein_[M.musculus] | | | | |
| AI596800_ESTs | −2.0 | −1.42466˜ | −1.0 | −1.1 |
| AA638908_ESTs | −2.0 | −1.0 | 1.0 | −1.1 |
| AI605657_serine/arginine-rich_protein_specific_kinase_2 | −2.0 | 1.0 | −1.4 | −1.1 |
| AA795130_ESTs,_Moderately_similar_to_S12207_hypo- | −2.0 | 1.1 | −1.1 | −1.0 |
| thetical_protein_[M.musculus] | | | | |
| AA146478_M.musculus_mRNA_(3C10)_for_IgA_V-D-J- | −2.0 | −2.4 | −1.1 | −1.4 |
| heavy_chain | | | | |
| AA397221_ESTs,_Weakly_similar_to_NED4_MOUSE_NEDD- | −2.0 | −1.0 | −1.2 | 1.1 |
| 4_PROTEIN_[M.musculus] | | | | |
| AA717269_ESTs,_Weakly_similar_to_T46456_hypo- | −2.0 | 1.0 | 1.0 | 1.1 |
| thetical_protein_DKFZp4346132.1_[H.sapiens] | | | | |
| AI666289_ESTs | −2.0 | −1.0 | −1.2 | 1.1 |
| AI552745_Mus_musculus,_clone_MGC:6888,_mRNA,_com- | −2.0 | 1.1 | −1.2 | −1.5 |
| plete_cds | | | | |
| W406908_anterior_gradient_2_(Xenopus_laevis) | −2.0 | −1.4 | 1.4 | −1.0 |
| AI892534_ribosomal_protein_S24 | −2.0 | −1.1 | 1.3 | −1.2 |
| AA450438_Mus_musculus_mRNA_for_erythroid_dif- | −2.0 | −1.3 | −1.7 | −1.3 |
| ferentiation_regulator,_partial | | | | |
| AA619114_solute_carrier_family_11_(proton- | −2.0 | −1.4 | −1.5 | 1.4 |
| coupled_divalent_metal_ion_transporters),_member_2 | | | | |
| AA608285_ubiquitin_fusion_degradation_1_like | −2.0 | 1.0 | −1.1 | −1.1 |
| AA549540_ESTs | −2.0 | 1.0 | 1.4 | 1.3 |
| AA066867_RIKEN_cDNA_3010002H13_gene | −2.0 | 1.48546˜ | 1.4 | −7.9 |
| AI608085_ESTs | −2.0 | −1.0 | −1.0 | −1.0 |
| AA474446_ESTs | −2.0 | 1.01705+˜ | 1.3 | 1.1 |
| AA444672_RIKEN_cDNA_2510001A17_gene | −2.0 | −1.1 | 1.3 | −1.1 |
| AA473972_heterogeneous_nucluar_ribonucleoprotein_H1 | −2.0 | −1.17495˜ | −1.2 | 1.0 |
| AA267106_ESTs | −2.0 | −1.2 | 1.3 | 1.1 |
| AA638929_ESTs,_Moderately_similar_to_S12207_hypo- thetical_protein_[M.musculus] | −2.0 | 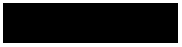 | −1.1 | −1.1 |
| AI391001_Public_domain_EST | −2.0 | −1.1 | −1.0 | 1.1 |
| AA575047_ESTs | −2.0 | 1.3 −1.2 | −1.1 | |
| AI450530_ESTs | −2.0 | 1.2 | 1.0 | −1.1 |
| AA536789_RIKEN_cDNA_1110002B05_gene | −2.0 | 1.3 | 1.1 | −1.2 |
| AA684191_CDC-like_kinase | −2.0 | −1.4 | −1.3 | 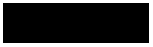 |
| AA549591_ESTs,_Highly_similar_to_CRM1_[H.sapiens] | −2.0 | 1.4 | −1.2 | −1.0 |
| AI536183_Public_domain_EST | −2.0 | 1.0 | −1.4 | 1.1 |
| AA874133_ESTs | −2.0 | 1.3 | −1.1 | −1.1 |
| AA684092_ESTs,_Moderately_similar_to_S12207_hypo- | −2.0 | −1.0 | 1.1 | 1.0 |
| thetical_protein_[M.musculus] | | | | |
| AA672766_Mouse_mRNA_for_TI-227 | −2.1 | 1.12734˜ | −1.1 | −1.1 |
| AA164064_ESTs | −2.1 | −1.14579˜ | −1.37792+ | 1.2 |
| W15606_dynein,_axon,_heavy_chain_11 | −2.1 | −1.8 | −1.1 | 1.0 |
| AA450914_RIKEN_cDNA_1810005K14_gene | −2.1 | −1.1 | 1.4 | −1.1 |
| AA896036_ESTs,_Highly_similar_to_GUANINE_NUCLEOTIDE- BINDING_PROTEIN_BETA_SUBUNIT- LIKE_PROTEIN_12.3_[M.musculus] | −2.1 | 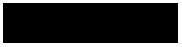 | 1.1 | −1.1 |
| AA914844_Public_domain_EST | −2.1 | 1.0 | 1.0 | 1.2 |
| AA059886_protein_phosphatase,_EF_hand_calcium- | −2.1 | 1.1 | 1.02202* | 1.2 |
| binding_domain_2 | | | | |
| AA067797_heat_shock_protein_cognate_70 | −2.1 | −1.5 | −1.1 | 1.3 |
| AI530648_ESTs | −2.1 | 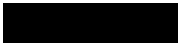 | 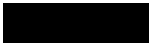 | −1.2 |
| AA619915_RIKEN_cDNA_1110015M06_gene | −2.1 | −1.0 | 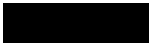 | 1.3 |
| AA000282_naked_cuticle_1_homolog_(Drosophila) | −2.1 | 1.0 | 1.0 | 1.3 |
| AA607713_Mus_musculus_10_day_old_male_pan- | −2.1 | −1.5 | −1.7 | 1.4 |
| creas_cDNA,_RIKEN_full- | | | | |
| length_enriched_library,_clone:1810009A17,_full_insert_sequence | | | | |
| AA152940_immunoglobulin_kappa_chain_variable_28_(V28) | −2.1 | −3.1 | −2.2 | −1.3 |
| AI640116_ESTs | −2.1 | 1.37609˜ | 1.3 | −1.2 |
| AA260035_ESTs | −2.1 | −1.1 | −1.2 | 1.2 |
| AA444576_heat_shock_protein,_60_kDa | −2.2 | −12 | 1.1 | 1.0 |
| AI451754_ESTs | −2.2 | 13 | −1.1 | 1.3 |
| AI019837_Iaminin,_beta_3 | −2.2 | −2.16452˜ | 1.21069+ | 1.5 |
| AA066404_topoisomerase_(DNA)_II_beta | −2.2 | 1.0 | −12 | −1.5 |
| AA880295_immunoglobulin_joining_chain | −2.2 | −2.4 | −1.9 | −1.1 |
| AA200306_ESTs | −2.2 | −1.06091+˜ | 1.1 | −1.2 |
| AI645264_ESTs,_Weakly_similar_to_S29170_an- | −101 | −1.19222˜ | 1.5 | −1.1 |
| nexin_VII_-_mouse_[M.musculus] | | | | |
| W56995_hemoglobin_X,_alpha- | −2.2 | 1.11232˜ | −2.1 | −2.0 |
| like_embryonic_chain_in_Hba_complex | | | | |
| AA288278_ESTs | −2.2 | −1.01004 | 1.4 | 1.0 |
| AI463733_ESTs,_Moderately_similar_to_S12207_hypo- | −2.2 | −1.1 | 1.2 | 1.1 |
| thetical_protein_-_mouse_[M.musculus] | | | | |
| AA152636_integrin_alpha_4 | −2.2 | 1.1 | −1.0 | 1.3 |
| AA138854_ESTs,_Weakly_similar_to_lyso- | −2.3 | −2.1 | 1.1 | −1.7 |
| phospholipase_I_[M.musculus] | | | | |
| AI117296_Paneth_cell_enhanced_expression | −2.3 | −1.28142˜ | −1.6 | 1.2 |
| AI592114_molybdenum_cofactor_synthesis_2 | −2.3 | 1.2 | −1.1 | 1.3 |
| AA435317_ESTs | −2.3 | 1.1 | 1. | −1.1 |
| AA105092_Bruton_agammaglobulinemia_tyrosine_kinase | −2.3 | 1.05832˜ | −1.53017+ | −1.4 |
| [symbol] | −2.4 | −1.6 | 1.0 | −1.3 |
| regulated_protein,_78kD) | | | | |
| AA466153_ESTs | −2.4 | −1.16147+˜ | 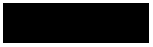 | 1.03478+ |
| AA718696_ESTs | −2.4 | −1.2 | −1.2 | −1.4 |
| AA212405_dihydrofolate_reductase | −2.5 | −2.6 | 1.1 | −1.4 |
| AA727065_ESTs | −2.5 | 1.2 | −1.2 | −1.6 |
| AI464865_ESTs | −2.5 | −1.21294+˜ | −1.1 | −1.4 |
| AI627143_ESTs | −2.5 | −3.6 | 1.2 | −1.1 |
| AA065764_AXL_receptor_tyrosine_kinase | −2.6 | 1.01562˜ | 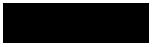 | −1.1 |
| AI390861_retinoic_acid_induced_1 | −2.6 | 1.01664+˜ | −1.1 | −1.3 |
| AA619964_nuclear_receptor_coactivator_4 | −2.7 | −1.0 | 1.1 | −1.1 |
| AA498574_kallikrein_6 | −2.7 | −2.3 | −1.2 | 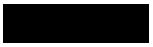 |
| AI596527_ribosomal_protein_L26 | −2.7 | −1.2 | 1.2 | −1.2 |
| AA521693_Public_domain_EST | −2.7 | −1.5 | −1.2 | −1.0 |
| AA210505_Public_domain_EST | −2.7 | −1.7 | −1.1 | 1.1 |
| AA242360_cytochrome_P450,_1a2,_aro- | −2.8 | −1.33495˜ | −1.37353+ | −2.4 |
| matic_compound_inducible | | | | |
| AI536352_G_protein- | −2.9 | −1.3 | −1.6 | −1.1 |
| coupled_receptor,_family_C,_group_5,_member_B | | | | |
| AA693081_alkaline_phosphatase_5 | −2.9 | 1.52048+˜ | 1.07417+ | −1.01637+ |
| AA105866_glytathione_S-transferase,_alpha_4 | −3.0 | 1.3 | 1.2 | 1.0 |
| AI425738_ESTs,_Weakly_simi- | −3.2 | 1.0 | −1.0 | −1.1 |
| lar_to_ACRO_HUMAN_ACROSIN_PRECURSO_[H.sapiens] | | | | |
| AA790247_RIKEN_cDNA_2210407P13_gene | −3.5 | −1.03402+˜ | −1.06819+ | 1.2 |
| AI614443_3′-phosphoadenosine_5′-phosphosulfate_synthase_2 | −3.7 | −1.4 | −1.3 | 1.4 |
| AA839435_cathepsin_E | −4.1 | −1.6 | −1.5 | −1.1 |
| AI605648_small_proline-rich_protein_2A | −5.5 | −3.9 | 1.0 | −1.2 |
| AI327450_phospholipase_A2,_group_IB,_pancreas | 1.01624˜ | −2.3 | −1.6 | −1.4 |
| AA674409_pancreatic_lipase_related_protein_1 | −1.0 | 1.2 | 1.0 | −1.1 |
| AA497618_crp-ductin | −5.0 | 1.5 | −1.2 | −1.3 |
| AA107101_prostate_stem_cell_antigen | 1.51614˜ | −1.8 | −2.1 | −1.0 |
| W12985_annexin_A10 | −1.03567˜ | −1.3 | −1.1 | 1.1 |
| AI876997_carboxyl_ester_lipase | −1.9 | −1.3 | −1.4 | −1.1 |
| AA982254_RIKEN_cDNA_2210010C04_gene | −1.3 | 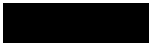 | −1.5 | −2.0 |
| AA237793_rat_regenerating_islet-derived,_mouse_homolog_1 | −2.2 | 1.0 | −1.4 | −2.7 |
| AA675111_RIKEN_cDNA_1810007A24_gene | −1.5 | 1.0 | −1.4 | −2.2 |
| AI451862_RIKEN_cDNA_0910001A18_gene | −2.6 | 1.2 | −1.3 | −2.4 |
| AI386046_trypsin_4 | −2.6 | 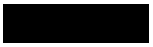 | −1.5 | −2.5 |
| AA790398_ribonuclease_1,_pancreatic | −3.3 | 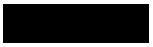 | −1.9 | −4.1 |
| AA675084_elastase_2 | −2.2 | 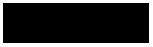 | −1.8 | −4.0 |
| AA717025_Mus_musculus_10_day_old_male_pan- creas_cDNA,_RIKEN_full- length_enriched_library,_clone:1810009A17,_full_insert_sequence | −5.2 | 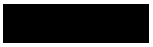 | −1.6 | −2.9 |
| AA821884_amylase_2,_pancreatic | −4.0 | 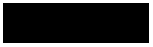 | −1.2 | −4.4 |
| AI386257_calbindin-D9k | −4.1 | 1.0 | −1.2 | −1.1 |
| AI894032_trefoil_factor_2_(spasmolytic_protein_1) | −1.89777˜ | −1.4 | 1.0 | −1.4 |

INTESTINE

| 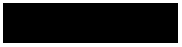 | blad | kidn | pl/ut | mam |
|---|---|---|---|---|
| Description | PL-9B_BDEnor | PL-10B_BDEnor | PL-11B_BDEnor | PL-12B_BDEnor |
| 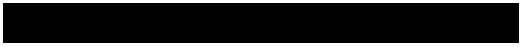 | 1.0 | −1.2 | 1.3 | 1.1 |
| 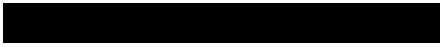 | 1.2 | 1.1 | 1.0 | 1.3 |
| 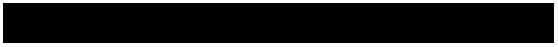 | 1.3 | 1.2 | 1.1 | −1.2 |
| 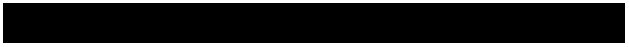 | −1.2 | 1.2 | −1.3 | 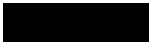 |
| 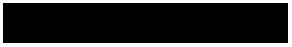 | 1.1 | 1.1 | −1.1 | 1.5 |
| 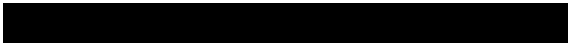 | 1.3 | 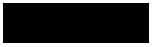 | −1.1 | 1.0 |
| 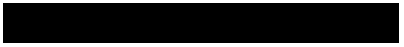 | −1.1 | 1.0 | 1.0 | −1.3 |
| 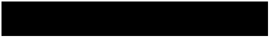 | 1.5 | 1.2 | 1.3 | 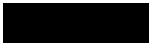 |
| 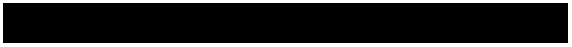 | −1.0 | 1.3 | −1.2 | −1.2 |
| 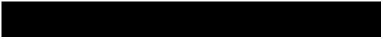 | 1.3 | 1.2 | 1.1 | 1.2 |
| 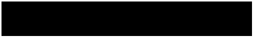 | −1.2 | −1.0 | 1.1 | 1.4 |
| 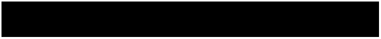 | 1.2 | −1.2 | 1.1 | 1.4 |
| 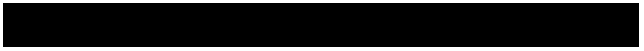 | −9.2 | −1.0 | 1.0 | 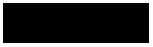 |
| 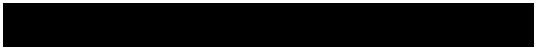 | −1.0 | 1.3 | 1.0 | −1.0 |
| 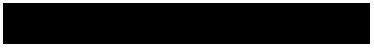 | −1.3 | 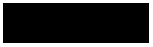 | −1.9 | −1.2 |
| 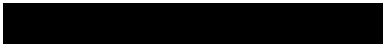 | −1.2 | −2.1 | −2.5 | −1.6 |
| AA871663_defensin_related_cryptdin,_related_sequence_7 | −1.1 | −1.5 | 1.1 | 1.2 |
| AA793433_glutamyl_aminopeptidase | 1.5 | 1.0 | 1.2 | −1.1 |
| AA869166_defensin_related_cryptdin,_related_sequence_10 | 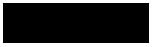 | 1.1 | 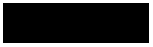 | 1.2 |
| AI324398_glycosylphosphatidylinositol_specific_phospholipase_D1 | −1.3 | 1.2 | −1.6 | −1.6 |
| AA066225_selenoprotein_P,_plasma,_1 | −1.1 | 1.2 | −1.6 | −1.1 |
| AA024217_Public_domain_EST | −1.8 | −1.2 | −2.3 | 1.1 |
| AA882450_RIKEN_cDNA_1200011D11_gene | 1.1 | −1.2 | −1.2 | 1.1 |
| AI385503_cysteine_rich_intestinal_protein | 1.0 | −1.0 | −1.0 | 1.1 |
| AA162211_guanine_nucleotide_binding_protein,_alpha_11 | 1.2 | 1.1 | 1.0 | 1.0 |
| AA415344_histone_deacetylase_7 | 1.0 | 1.4 | −1.2 | 1.0 |
| AA239419_P_glycoprotein_2 | 1.1 | −1.0 | −1.2 | 1.3 |
| AA109528_ESTs,_Weakly,_simi- | −1.2 | 1.4 | −1.1 | 1.0 |
| lar_to_CA1C_MOUSE_COLLAGEN_AL- | | | | |
| PHA_1(XII)_CHAIN_PRECURSOR_[M.musculus] | | | | |
| AA770902_actin,_alpha_1,_skeletal_muscle | 1.1 | 1.1 | −1.1 | −1.2 |
| AA647336_cytochrome_P450,_steroid_inducible_3a11 | −1.0 | −1.1 | 1.2 | 1.1 |
| AA386758_ESTs | 1.1 | 1.1 1.1 | −1.0 | |
| AA871641_defensin_related_cryptdin,_related_sequence_2 | 1.2 | 1.1 | 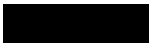 | 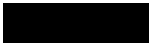 |
| AA871410_defensin_related_cryptdin_5 | 1.2 | −1.1 | 1.1 | 1.0 |
| AA2102337_Mus_musculus,_clone_MGC:6377,_mRNA,_com- | 1.2 | 1.4 | 1.0 | −1.0 |
| plete_cds | | | | |
| AI552087_IQ_motif_containing_GTPase_activating_protein_1 | 1.0 | −1.0 | 1.0 | 1.1 |
| AA166427_Mus_musculus_germline_immunoglobulin_gam- | 1.2 | −1.1 | 1.1 | −1.5 |
| ma_constant_region_(IgG3)_mRNA | | | | |
| AA756136_actin,_gamma_2,_smooth_muscle,_enteric | 1.1 | 1.1 | −1 .3 | 1.1 |
| AA239727_ATP-binding_cassette, sub- family_B_(MDR/TAP),_member_1 | −1.0 | −1.2 | −1.4 | 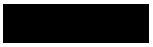 |
| AA469630_choline_kinase | 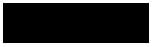 | 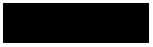 | 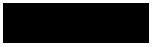 | 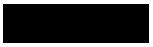 |
| AI594147_beta-2_microglobulin | −1.0 | 1.3 | −1.1 | −1.2 |
| AA684403_ESTs | −1.3 | −1.3 | −1.1 | −1.1 |
| AA869219_intelectin | 1.2 | −1.0 | 1.5 | 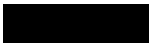 |
| AA073925_ESTs | 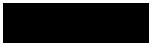 | 1.2 | 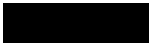 | 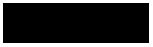 |
| W54384_glutathione_s-transferase,_mu_2 | 1.2 | 1.3 | 1.2 | 1.0 |
| AA013726_cathepsin_J | 1.2 | 1.1 | −2.1 | 1.5 |
| AI549624_RIKEN_cDNA_0610041E09_gene | 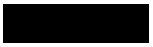 | 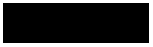 | 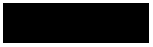 | 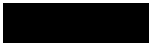 |
| AI552688_ESTs,_Weakly_simi- | −1.0 | −1.1 | −1.5 | 1.1 |
| lar_to_MIA_MOUSE_MELANOMA_DERIVED_GROWTH_REG- | | | | |
| ULATORY_PROTEIN_PRUCURSOR_[M.musculus] | | | | |
| AA450725_membrane_metallo_endopeptidase | −1.4 | 1.1 | 1.0 | 1.3 |
| AA870247_mitogen_regulated_protein,_proliferin_3 | 1.1 | 1.0 | −1.8 | 1.4 |
| AI892437_transglutaminase_2,_C_polypeptide | −1.2 | 1.1 | −1.1 | −1.1 |
| AI036411_angiotensin_converting_enzyme | 1.1 | 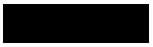 | 1.2 | −1.3 |
| AA619767_ornithine_decarboxylase_antizyme | −1.1 | 1.1 | −1.3 | −1.2 |
| AA106623_hexosaminidase_B | 1.1 | 1.0 | −1.1 | −1.1 |
| A14224_N-myc_downstream_regulated_1 | 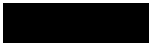 | 1.0 | 1.3 | 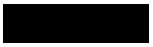 |
| AA624460_actin,_alpha_2,_smooth_muscle,_aorta | 1.1 | 1.0 | −1.2 | 1.0 |
| W36474_metallothionein_2 | −1.1 | 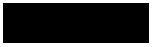 | −1.9 | 1.0 |
| AA821963_aspartyl_aminopeptidase | 1.0 | 1.3 | 1.0 | −1.2 |
| W13521_cytochrome_P450,_2j6 | 1.2 | −1.0 | 1.4 | 1 .2 |
| AA413490_transferin_receptor | 1.2 | −1.3 | 1.5 | 1.5 |
| AI594093_Mus_musculus_putative_CAMP_pro- | −1.0 | −1.2 | 1.2 | −1.3 |
| tein_(Camp)_mRNA,_complete_cds | | | | |
| AA028420_small_proline-rich_1A | 1.0 | −1.1 | 1.3 | 1.3 |
| AA245987_ESTs | −1.00306+ | 1.2 | 1.28207+ | 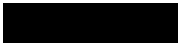 |
| AI390129_proteasome_(prosome,_macropain)_sub- | 1.2 | 1.0 | 1.0 | −1.3 |
| unit,_alpha_type_2 | | | | |
| AI156982_nuclear_receptor_subfamily_5,_group_A,_member_2 | −1.2 | −1.2 | −1.5 | −1.2 |
| AA546964_ESTs,_Weakly_simi- | −1.4 | −1.5 | 1.2 | −1.0 |
| lar_to_TLM_MOUSE_TLM_PROTEIN_[M.musculus] | | | | |
| AI605616_ESTs | −1.2 | −1.2 | −1.5 | −1.1 |
| AA561934_ESTs | −1.1 | −1.5 | 1.0 | 1.0 |
| AI157093_RIKEN_cDNA_2700094F01_gene | −1.3 | −1.4 | 1.0 −1.1 | |
| AA794319_ESTs | −1.3 | −1.0 | −1.1 | −1.1 |
| AA433804_ESTs | −1.2 | −1.4 | 1.0 | −1.1 |
| AA754696_immuno- | −1.2 | −1.0 | −1.1 | −1.4 |
| globulin_heavy_chain_6_(heavy_chain_of_IgM) | | | | |
| W09198_calcium_binding_protein_A6_(calcyclin) | 1.0 | 1.2 | −1.2 | 1.0 |
| AA967824_arginine_vasopressin | −1.3 | 1.4 | −1.2 | −2.3 |
| AW496194_immunoglobulin_kappa_chain_variable_20_(V20_family) | −1.0 | 1.4 | −1.0 | −2.7 |
| AA647876_ESTs | −1.2 | −1.3 | 1.0 | −1.0 |
| AA790455_RIKEN_cDNA_3110001I20_gene | −1.1 | 1.0 | 1.2 | 1.3 |
| AA068562_RIKEN_cDNA_2810417H13_gene | −1.2 | −1.5 | −1.1 | −1.2 |
| AI627053_ESTs | −1.1 | −1.4 | 1.1 | 1.0 |
| AI607043_ESTs,_Weakly_similar_to_S12207_hypo- | −1.1 | −1.3 | 1.1 | −1.2 |
| thethical_protein_[M/musculus] | | | | |
| AI536309_RIKEN_cDNA_2210410L06_gene | −1.6 | −2.1 | −1.5 | −2.1 |
| AA166386_transducer_of_ErbB-2.1 | −1.1 | −1.0 | −1.2 | 1.2 |
| AA414465_ESTs | −1.2 | −1.3 | 1.0 | −1.1 |
| AA529377_heat_shock_protein,_86_kDa_1 | 1.2 | −1.0 | −1.0 | 1.3 |
| AA562129_ESTs,_Weakly_similar_to_T23699_hypo- | −1.0 | 1.0 | −1.1 | −1.1 |
| thetical_protein_M04B2.4_-_Caenorhabditis_elegans_[C.elegans] | | | | |
| AI428582_Public_domain_EST | −1.1 | −1.1 | 1.1 | −1.1 |
| AA738642_proteasome_(prosome,_macropain)_sub- | 1.2 | −1.1 | 1.1 | −1.2 |
| unit,_alpha_type_3 | | | | |
| AI596504_t-complex_testis_expressed_1 | −1.0 | −1.1 | 1.3 | −1.0 |
| AI036487_ESTs | −1.0 | −1.3 | 1.1 | 1.0 |
| AI386181_gamma-glutamyl_carboxylase | −1.2 | −1.3 | −1.0 | −1.2 |
| AA422743_ESTs,_Weakly_similar_to_AF091622_1_PHD_fin- | −1.1 | −1.2 | −1.1 | −1.1 |
| ger_protein_3_[H.sapiens] | | | | |
| AA289639_ESTs | −1.1 | −1.2 | 1.1 | −1.1 |
| W84257_interferon_(alpha_and_beta)_receptor | −1.1 | −1.3 | 1.1 | −1.1 |
| AI429064_ESTs,_Weakly_similar_to_S12207_hypo- | −1.0 | −1.2 | 1.0 | −1.3 |
| thetical_protein_[M.musculus] | | | | |
| AA197547__RIKEN_cDNA_2310031L18_gene | −1.1 | −1.4 | 1.0 | −1.1 |
| AA563376_DNA_segment,_Chr_8,_ERATO_Doi_233,_expressed | −1.1 | −1.3 | 1.1 | 1.0 |
| AA414730_NCK-associated_protein_1 | 1.2 | 1.1 | 1.1 | 1.1 |
| AA276909_ESTs,_Weakly_simi- | 1.1 | −1.1 | 1.3 | 1.3 |
| lar_to_GRBA_MOUSE_GROWTH_FACTOR_RECEPTOR- | | | | |
| BOUND_PROTEIN_10_[M/musculus] | | | | |
| AA638300_ESTs,_Moderately_similar_to_S12207_hypo- | −1.1 | −1.3 | 1.0 | −1.1 |
| thetical_protein_[M.musculus] | | | | |
| W34620_kallikrein_26 | 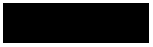 | 1.1 | −1.1 | −1.4 |
| AI592338_ESTs | −1.0 | −1.1 | −1.0 | 1.1524+ |
| AA727388_ESTs,_Moderately_similar_to_S12207_hypo- | −1.1 | −1.4 | −1.0 | −1.2 |
| thetical_protein_[M.musculus] | | | | |
| AA437755_thymus_expressed_acidic_protein | −1.5 | −1.5 | −1.0 | 1.1 |
| AA981354_cyclin_T1 | −1.3 | −1.3 | −1.1 | −1.2 |
| AI465828_ESTs | −1.2 | −1.3 | 1.2 | −1.3 |
| AA467531_double_C2,_gamma | −1.2 | −1.0 | −1.0 | 1.1 |
| AA856528_ESTs,_Highly_similar_to_S12207_hypo- | −1.2 | −1.4 | −1.0 | −1.2 |
| thetical_protein_[M.musculus] | | | | |
| [symbol] | 1.17519+ | −1.1 | 1.0 | −1.4 |
| AA673741_DNA_segment,_Chr_12,_ERATO_Doi_604,_expressed | −1.2 | −1.4 | −1.3 | −1.3 |
| AA619869_RIKEN_cDNA_5730534O06_gene | 1.0 | −1.5 | 1.0 | −1.2 |
| AI390319_ESTs,_Weakly_similar_to_apolipoprotein_F_[H.sapiens] | −1.1 | −1.2 | 1.1 | −1.3 |
| AI614000_ESTs | −1.1 | −1.2 | −1.0 | −1.1 |
| AA794176_ESTs,_Moderately_similar_to_SPA- | −1.3 | −1.3 | 1.1 | −1.1 |
| 1_like_protein_p1294_[R.norvegicus] | | | | |
| AI156378_ESTs | 1.3 | 1.4 | −1.0 | 1.1 |
| AA656612_RIKEN_cDNA_4631416G20_gene | −1.2 | −1.4 | −1.0 | −1.1 |
| AA177238_ESTs | 1.0 | −1.2 | 1.2 | −1.1 |
| AA023331_adenosine_deaminase | 1.3 | 1.1 | −1.1 | 1.4 |
| AA415510_ESTs | −1.2 | −1.2 | −1.1 | −1.1 |
| AI447943_EST | −1.2 | −1.5 | −1.0 | −1.2 |
| AA623448_ESTs,_Weakly_similar_to_NED5_MOUSE_NEDD- | −1.1 | −1.3 | 1.1 | −1.2 |
| 4_PROTEIN_[M.musculus] | | | | |
| AI120315_splicing_factor,_arginine/serine- | 1.1 | −1.2 | −1.1 | −1.4 |
| rich_5_(SRp40,_HRS) | | | | |
| AA509560_ESTs | −1.2 | −1.2 | −1.0 | −1.3 |
| AA692960_ESTs,_Weakly_similar_to_AF089896_1_topoisomerase- | −1.1 | −1.2 | 1.0 | −1.3 |
| related_function_protein_[H.sapiens] | | | | |
| AA469667_tumor_rejection_antigen_gp96 | 1.2 | −1.1 | 1.1 | −1.0 |
| AA982569_ESTs | −1.2 | −1.5 | −1.0 | −1.2 |
| W14153_ESTs,_Moderately_similar_to_S12207_hypo- | −1.1 | −1.4 | 1.0 | 1.0 |
| thetical_protein_[M.musculus] | | | | |
| AI596800_ESTs | −1.3 | −1.4 | −1.1 | −1.2 |
| AA638908_ESTs | −1.2 | −1.4 | −1.0 | −1.1 |
| AI605657_serine/arginine-rich_protein_specific_kinase_2 | −1.0 | −1.0 | −1.2 | −1.1 |
| AA795130_ESTs,_Moderately_similar_to_S12207_hypo- | −1.1 | −1.3 | −1.1 | −1.2 |
| thetical_protein_[M.musculus] | | | | |
| AA146478_M.musculus_mRNA_(3C10)_for_IgA_V-D-J- heavy_chain | 1.5 | 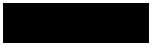 | 1.1 | 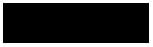 |
| AA397221_ESTs,_Weakly_similar_to_NED4_MOUSE_NEDD- | 1.0 | −1.2 | 1.1 | 1.0 |
| 4_PROTEIN_[M.musculus] | | | | |
| AA717269_ESTs,_Weakly_similar_to_T46456_hypo- | −1.1 | −1.2 | 1.1 | −1.2 |
| thetical_protein_DKFZp4346132.1_[H.sapiens] | | | | |
| AI666289_ESTs | −1.2 | −1.2 | −1.0 | −1.1 |
| AI552745_Mus_musculus,_clone_MGC:6888,_mRNA,_com- | −1.2 | −1.4 | −1.3 | −1.1 |
| plete_cds | | | | |
| W406908_anterior_gradient_2_(Xenopus_laevis) | −1.0 | −1.0 | −1.0 | −1.2 |
| AI892534_ribosomal_protein_S24 | 1.2 | −1.4 | −1.1 | −1.2 |
| AA450438_Mus_musculus_mRNA_for_erythroid_dif- | −1.3 | −1.6 | −1.2 | −1.2 |
| ferentiation_regulator,_partial | | | | |
| AA619114_solute_carrier_family_11_(proton- | 1.1 | −1.0 | 1.1 | −1.0 |
| coupled_divalent_metal_ion_transporters),_member_2 | | | | |
| AA608285_ubiquitin_fusion_degradation_1_like | −1.2 | −1.3 | −1.1 | −1.1 |
| AA549540_ESTs | −1.1 | −1.4 | 1.2 | 1.1 |
| AA066867_RIKEN_cDNA_3010002H13_gene | −1.1 | −1.1 | −1.7 | −1.2 |
| AI608085_ESTs | −1.3 | −1 .4 | 1.0 | −1.2 |
| AA474446_ESTs | −1.1 | −1.3 | 1.2 | −1.1 |
| AA444672_RIKEN_cDNA_2510001A17_gene | 1.2 | −1.4 | 1.0 | −1.2 |
| AA473972_heterogeneous_nucluar_ribonucleoprotein_H1 | 1.1 | −1.5 | −1.1 | 1.1 |
| AA267106_ESTs | −1.1 | −1.1 | 1.1 | −1.2 |
| AA638929_ESTs,_Moderately_similar_to_S12207_hypo- | −1.1 | −1.3 | −1.0 | −1.2 |
| thetical_protein_[M.musculus] | | | | |
| AI391001_Public_domain_EST | −1.1 | −1.4 | 1.2 | −1.0 |
| AA575047_ESTs | −1.4 | −1.1 | 1.0 | −1.0 |
| AI450530_ESTs | −1.3 | −1.4 | −1.1 | −1.1 |
| AA536789_RIKEN_cDNA_1110002B05_gene | 1.0 | −1.1 | −1.1 | 1.0 |
| AA684191_CDC-like_kinase | 1.4 | −2.1 | 1.4 | 1.2 |
| AA549591_ESTs,_Highly_similar_to_CRM1_[H.sapiens] | −1.4 | −1.4 | −1.0 | −1.1 |
| AI536183_Public_domain_EST | −1.0 | −1.2 | 1.1 | −1.1 |
| AA874133_ESTs | −1.2 | −1.4 | −1.0 | −1.2 |
| AA684092_ESTs,_Moderately_similar_to_S12207_hypo- | −1 .2 | −1.4 | −1.1 | −1.2 |
| thetical_protein_[M.musculus] | | | | |
| AA672766_Mouse_mRNA_for_TI-227 | −1.0 | −1.3 | −1.1 | −1.2 |
| AA164064_ESTs | 1.0 | −1.1 | 1.1 | 1.0 |
| W15606_dynein,_axon,_heavy_chain_11 | −1.1 | −1.2 −1.2 | −1.1 | |
| AA450914_RIKEN_cDNA_1810005K14_gene | −1.1 | 1.1 | 1.1 | −1.1 |
| AA896036_ESTs,_Highly_similar_to_GUANINE_NUCLEOTIDE- | −1.0 | −1.1 | −1.1 | −1.2 |
| BINDING_PROTEIN_BETA_SUBUNIT- | | | | |
| LIKE_PROTEIN_12.3_[M.musculus] | | | | |
| AA914844_Public_domain_EST | −1.1 | −1.3 | 1.1 | −1.1 |
| AA059886_protein_phosphatase,_EF_hand_calcium- | −1.1 | −1.3 | 1.1 | −1.1 |
| binding_domain_2 | | | | |
| AA067797_heat_shock_protein_cognate_70 | 1.5 | 1.3 | 1.0 | 1.2 |
| AI530648_ESTs | 1.0 | −1.1 | −1.0 | −1.1 |
| AA619915_RIKEN_cDNA_1110015M06_gene | 1.1 | −1.1 | 1.2 | −1.0 |
| AA000282_naked_cuticle_1_homolog_(Drosophila) | −1.1 | −1.3 | 1.2 | −1.1 |
| AA607713_Mus_musculus_10_day_old_male_pan- | −1.0 | −1.1 | 1.5 | 1.4 |
| creas_cDNA,_RIKEN_full- | | | | |
| length_enriched_library,_clone:1810009A17,_full_insert_sequence | | | | |
| AA152940_immunoglobulin_kappa_chain_variable_28_(V28) | −1.3 | 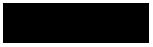 | −1.1 | −2.5 |
| AI640116_ESTs | −1.1 | 1.1 | −1.0 | 1.3 |
| AA260035_ESTs | −1.0 | −1.2 | 1.1 | −1.1 |
| AA444576_heat_shock_protein,_60_kDa | 1.1 | 1.0 | −1.1 | −1.2 |
| AI451754_ESTs | 1.0 | −1.1 | 1.2 −1.1 | |
| AI019837_Iaminin,_beta_3 | −1.1 | 1.1 | 1.1 | 1.4 |
| AA066404_topoisomerase_(DNA)_II_beta | −1.2 | −1.5 | −1.3 | −1.2 |
| AA880295_immunoglobulin_joining_chain | 1.3 | 1.5 | 1.4 | −1.3 |
| AA200306_ESTs | −1.3 | −1.3 | 1.1 | −1.1 |
| AI645264_ESTs,_Weakly_similar_to_S29170_an- | 1.3 | 1.5 | −1.1 | −1.1 |
| nexin_VII_-_mouse_[M.musculus] | | | | |
| W56995_hemoglobin_X,_alpha- | 1.0 | −1.2 | −4.0 | −1.6 |
| like_embryonic_chain_in_Hba_complex | | | | |
| AA288278_ESTs | −1.2 | −1.4 | 1.2 | 1.0 |
| AI463733_ESTs,_Moderately_similar_to_S12207_hypo- | −1.1 | −1.3 | 1.0 | −1.2 |
| thetical_protein_-_mouse_[M.musculus] | | | | |
| AA152636_integrin_alpha_4 | −1.3 | −1.4 | 1.2 | −1.0 |
| AA138854_ESTs,_Weakly_similar_to_lyso- | −1.0 | 1.4 | −1.1 | 1.2 |
| phospholipase_I_[M.musculus] | | | | |
| AI117296_Paneth_cell_enhanced_expression | −1.0 | −1.8 | 1.0 | −1.0 |
| AI592114_molybdenum_cofactor_synthesis_2 | 1.0 | −1.3 | 1.3 | 1.0 |
| AA435317_ESTs | −1.5 | −1.4 | 1.1 | −1.2 |
| AA105092_Bruton_agammaglobulinemia_tyrosine_kinase | 1.0 | −1.2 | −1.2 | −1.2 |
| [symbol] | 1.2 −1.1 | −1.1 | −1.2 | |
| regulated_protein,_78kD) | | | | |
| AA466153_ESTs | 1.2 | 1.3 | 1.1 | −1.33715+ |
| AA718696_ESTs | −1.3 | −1.4 | −1.4 | 1.2 |
| AA212405_dihydrofolate_reductase | −1.2 | 1.1 | −1.1 | −1.7 |
| AA727065_ESTs | −1.2 | −1.4 | −1.4 | −1.3 |
| AI464865_ESTs | −1.2 | −1.2 | −1.4 | 1.1 |
| AI627143_ESTs | −1.2 | −1.1 | −1.1 | −1.0 |
| AA065764_AXL_receptor_tyrosine_kinase | 1.1 | −1.1 | −1.1 | −1.1 |
| AI390861_retinoic_acid_induced_1 | −1.3 | −1.1 | −1.2 | −1.5 |
| AA619964_nuclear_receptor_coactivator_4 | 1.2 | 1.3 | −1.1 | −1.1 |
| AA498574_kallikrein_6 | 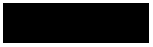 | 1.1 | 1.0 | 1.3 |
| AI596527_ribosomal_protein_L26 | 1.1 | −1.1 | −1.2 | −1.5 |
| AA521693_Public_domain_EST | −1.5 | −1.1 | −1.3 | −1.2 |
| AA210505_Public_domain_EST | 1.3 | 1.1 | 1.1 | 1.2 |
| AA242360_cytochrome_P450,_1a2,_aro- matic_compound_inducible | 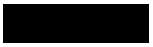 | 1.1 | 1.2 | 1.2 |
| AI536352_G_protein- | −1.3 | −1.1 | −1.1 | 1.0 |
| coupled_receptor,_family_C,_group_5,_member_B | | | | |
| AA693081_alkaline_phosphatase_5 | −1.07118+ | −1.16957+ | 1.08192+ | −1.03494+ |
| AA105866_glytathione_S-transferase,_alpha_4 | 1.3 | 1.3 | −1.1 | −1.2 |
| AI425738_ESTs,_Weakly_simi- | −1.3 | −1.3 | −1.2 | −1.3 |
| lar_to_ACRO_HUMAN_ACROSIN_PRECURSO_[H.sapiens] | | | | |
| AA790247_RIKEN_cDNA_2210407P13_gene | −1.0 | 1.1 | 1.1 | 1.3 |
| AI614443_3′-phosphoadenosine_5′-phosphosulfate_synthase_2 | 1.5 | 1.3 | 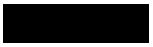 | 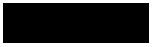 |
| AA839435_cathepsin_E | −1.1 | −1.3 | 1.1 | 1.0 |
| AI605648_small_proline-rich_protein_2A | −1.2 | −1.1 | −1.5 | −1.1 |
| AI327450_phospholipase_A2,_group_IB,_pancreas | −1.4 | −1.4 | −1.1 | −1.0 |
| AA674409_pancreatic_lipase_related_protein_1 | −1.1 | −1.1 | 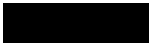 | 1.3 |
| AA497618_crp-ductin | −1.3 | −1.2 | 1.5 | 1.2 |
| AA107101_prostate_stem_cell_antigen | −1.0 | −1.1 | −1.4 | −1.4 |
| W12985_annexin_A10 | 1.1 | −1.0 | 1.0 | −1.9 |
| AI876997_carboxyl_ester_lipase | −1.1 | 1.1 | 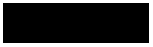 | 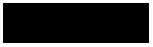 |
| AA982254_RIKEN_cDNA_2210010C04_gene | −2.0 | −1.4 | −1.1 | −1.1 |
| AA237793_rat_regenerating_islet-derived,_mouse_homolog_1 | −2.7 | −1.2 | −1.2 | −1.2 |
| AA675111_RIKEN_cDNA_1810007A24_gene | −2.2 | −1.2 | −1.2 | −2.0 |
| AI451862_RIKEN_cDNA_0910001A18_gene | −2.4 | −1.3 | −1.1 | −1.3 |
| AI386046_trypsin_4 | −2.5 | −1.1 | −1.3 | −1.2 |
| AA790398_ribonuclease_1,_pancreatic | −4.1 | −1.1 | −1.2 | −1.1 |
| AA675084_elastase_2 | −4.0 | −1.4 | −1.2 | −1.4 |
| AA717025_Mus_musculus_10_day_old_male_pan- | −2.9 | 1.2 | −1.1 | −1.4 |
| creas_cDNA,_RIKEN_full- | | | | |
| length_enriched_library,_clone:1810009A17,_full_insert_sequence | | | | |
| AA821884_amylase_2,_pancreatic | −4.4 | −1.1 | −1.1 | −1.2 |
| AI386257_calbindin-D9k | −1.1 | −1.2 | −1.2 | 1.4 |
| AI894032_trefoil_factor_2_(spasmolytic_protein_1) | −1.4 | −1.9 | −1.0 | 1.4 |

STOMACH

| 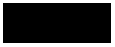 | spl | panc | liv | 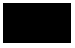 |
|---|---|---|---|---|
| Description | PL-4B_BDEnor | PL-2B_BDEnor | PL-6B_BDEnor | PL-8B_BDEnor |
| 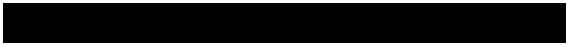 | 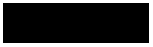 | 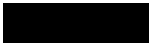 | −1.3 | 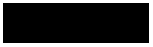 |
| 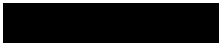 | 1.4 | 1.4 | −1.2 | 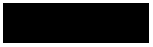 |
| 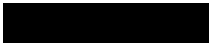 | 1.2 | 1.3 | −1.1 | 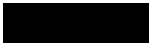 |
| 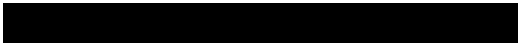 | 1.2 | 1.5 | 1.0 | 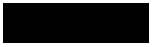 |
| 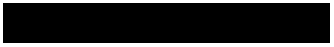 | 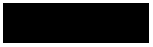 | 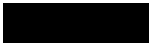 | −1.4 | 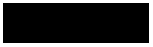 |
| 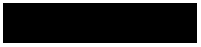 | 1.5 | 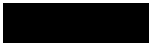 | 1.0 | 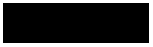 |
| 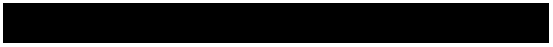 | 1.35465+ | 1.07934+ | 1.02095+ | 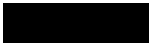 |
| 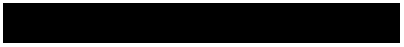 | 1.4 | 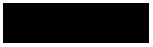 | −1.5 | 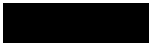 |
| 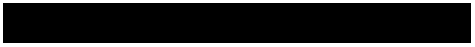 | 1.24627+ | 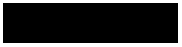 | −1.0038+ | 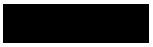 |
| 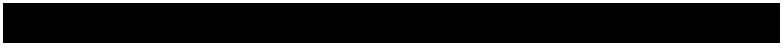 | 1.4 | 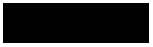 | −1.1 | 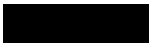 |
| 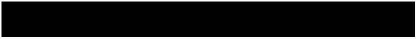 | 1.34106+ | 1.0 | 1.1 | 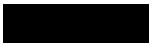 |
| 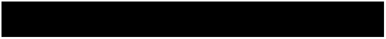 | 1.2 | 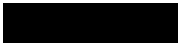 | −1.05799+ | 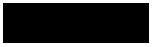 |
| 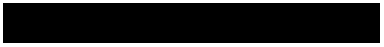 | 1.4 | 1.1 | −1.3 | 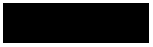 |
| 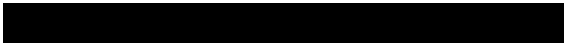 | 1.4 | 1.3 | −1.3 | 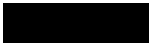 |
| 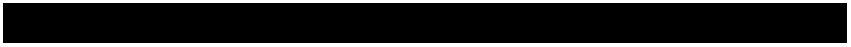 | −1.3 | 1.0 | −1.6 | 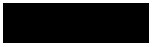 |
| 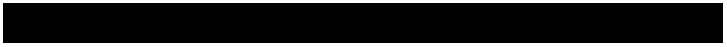 | 1.02567+ | 1.1 | −1.25592+ | 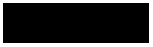 |
| 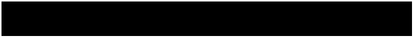 | 1.1 | 1.0 | −1.3 | 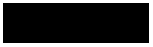 |
| 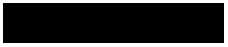 | 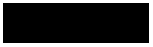 | 1.2 | −1.1 | 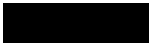 |
| 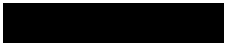 | 1.1 | 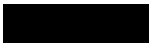 | −1.5 | 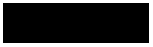 |
| 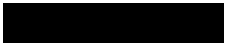 | 1.3 | 1.5 | −1.1 | 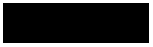 |
| 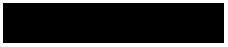 | 1.3 | 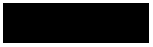 | −1.4 | 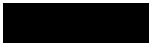 |
| 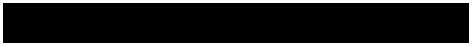 | 1.1 | 1.2 | −1.4 | 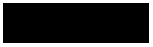 |
| 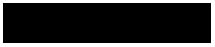 | 1.2 | 1.3 | −1.3 | 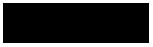 |
| 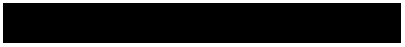 | −1.09414+ | 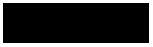 | −1.1 | 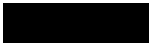 |
| 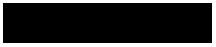 | 1.1 | 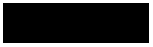 | 1.0557+ | 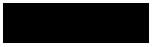 |
| 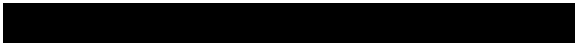 | 1.1 | 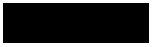 | −1.5 | 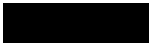 |
| 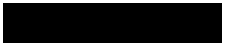 | 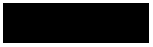 | 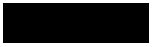 | −1.1 | 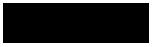 |
| 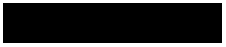 | 1.1 | 1.3 | −1.3 | 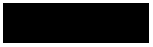 |
| 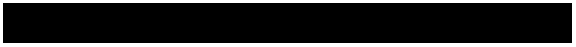 | 1.5 | 1.5 | 1.1 | 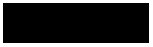 |
| 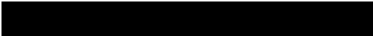 | 1.2 | 1.1 | −1.4 | 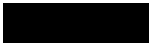 |
| 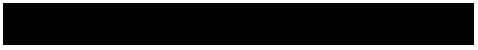 | 1.1 | 1.0 | −1.3 | 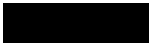 |
| 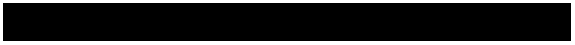 | 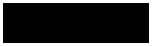 | 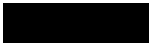 | −1.0 | 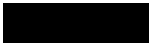 |
| 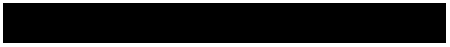 | 1.23774+ | 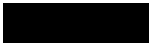 | −1.38204+ | 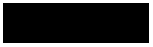 |
| 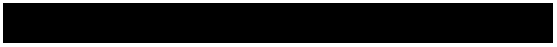 | 1.3 | 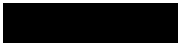 | 1.1 | 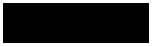 |
| 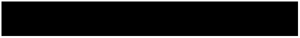 | 1.4 | 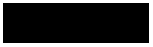 | −1.4 | 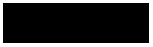 |
| 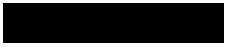 | 1.22006+ | 1.2 | −1.28881+ | 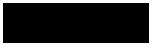 |
| 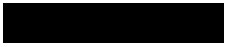 | 1.5 | 1.5 | −1.0 | 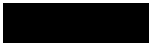 |
| 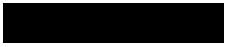 | 1.2 | 1.2 | −1.2 | 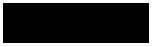 |
| 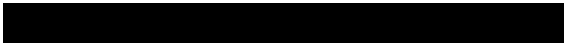 | 1.3 | 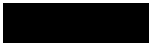 | −1.1 | 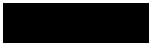 |
| AA638805_Public_domain_EST | 1.1 | 1.5 | −1.2 | 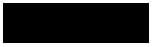 |
| AI550977_ESTs | −1.0 | 1.3 | −1.1 | 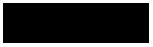 |
| AA683789_sirtuin_1_((silent_mating_type_information_regu- lation_2,_homolog)_1_(S._cerevisiae) | 1.1 | 1.3 | −1.2 | 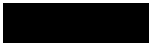 |
| AI549694_ESTs,_Highly_similar_to_P300_HUMAN_E1A- ASSOCIATED_PROTEIN_P300_[H.sapiens] | 1.0 | 1.1 | −1.3 | 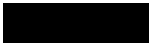 |
| AI550200_SEC61,_alpha_subunit_2_(S._cerevisiae) | 1.3 | 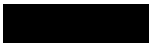 | 1.1 | 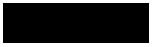 |
| AI645426_diaphanous_homolog_3_(Drosophila) | 1.2 | 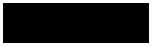 | −1.3 | 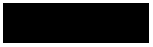 |
| AA231099_necdin | −1.8 | 1.3 | 1.1 | 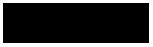 |
| AA530026_ESTs | 1.20192+ | 1.46056+ | 1.03838+ | 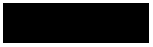 |
| AI552219_ESTs | 1.2087+ | 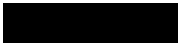 | −1.18563+ | 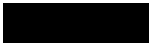 |
| AA517691_ESTs | 1.3 | 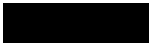 | −1.2 | 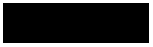 |
| AA517337_WASP_family_1 | 1.1 | 1.0 | −1.6 | 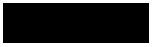 |
| AA543950_RIKEN_cDNA_2900073G15_gene | 1.1 | 1.4 | −1.2 | 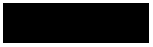 |
| AA617362_low_density_lipoprotein_receptor-related_protein_2 | 1.1 | 1.4 | −1.1 | 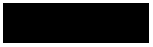 |
| AA473684_RIKEN_cDNA_C030002O17_gene | 1.22138+ | 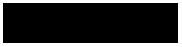 | −1.02182+ | 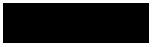 |
| AA265337_deoxyguanosine_kinase | 1.1 | 1.1 | −1.2 | 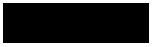 |
| AA588981_Mus_musculus_brain_cDNA,_clone_MNCb-1820 | 1.0 | 1.1 | −1.2 | 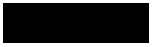 |
| AI325781_ESTs,_Weakly_similar_to_S37771_ankyrin,_erythro- cyte_-_mouse_[M.musculus] | 1.4 | 1.3 | −1.1 | 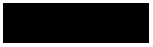 |
| AI549656_RIKEN_cDNA_3632413F13_gene | 1.2 | 1.4 | −1.22779* | 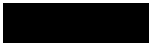 |
| AI549666_ESTs | 1.35481+ | 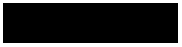 | 1.04863+ | 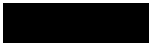 |
| AI425572_ESTs,_Highly_similar_to_zyginl_[R.norvegicus] | 1.3 | 1.2 | −1.2 | 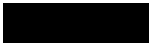 |
| AA210502_Public_domain_EST | 1.1 | 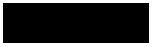 | −1.2 | 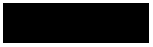 |
| AI509664_RIKEN_cDNA_2410174K12_gene | −1.0 | 1.1 | −1.1864+ | 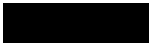 |
| AI587986_ESTs | 1.0 | 1.1 | −1.1 | 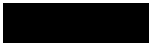 |
| AA606582_Public_domain_EST | 1.13444+ | 1.5 | −1.2 | 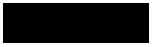 |
| AI551955_RIKEN_cDNA_6330415E02_gene | 1.0 | 1.3 | 1.4 | 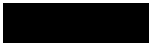 |
| AA823452_ESTs | 1.44959+ | 1.3508+ | −1.08199+ | 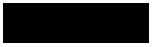 |
| AW210270_ESTs,_Weakly_similar_to_T17254_hypo- thetical_protein_KKFZp586O1022.1_[H.sapien] | 1.1 | 1.3 | −1.2 | 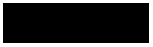 |
| AA412912_ESTs | 1.2 | 1.5 | −1.4 | 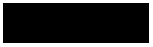 |
| AA068624_hydroxyacid_oxidase_(glycolate_oxidase)_3 | 1.3 | 1.3 | −1.2 | 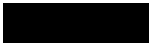 |
| AA096985_chtochrome_P450,_2j5 | 1.0 | −1.0 | −1.0 | 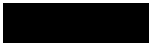 |
| AA709576_ESTs | 1.1 | 1.1 | −1.44348+ | 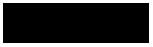 |
| AA611551_Mest-linked_imprinted_transcript_1 | 1.27918+ | 1.1 | −1.0 | 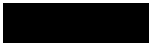 |
| AA000370_RIKEN_cDNA_4933438K12_gene | 1.3 | 1.1 | −1.5 | 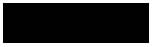 |
| AA153231_Public_domain_EST | 1.3 | 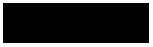 | 1.0 | 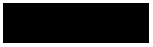 |
| AA288247_ESTs,_Weakly_similar_to_MRP5_MOUSE_MULTI- DRUG_RESISTANCE-ASSOCIATED_PROTEIn_5_[M.musculus] | 1.1 | 1.4 | −1.1 | 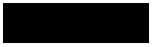 |
| AA709688_ESTs | 1.2 | 1.2 | −1.1503+ | 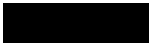 |
| AA615213_ESTs,_Moderately_similar_to_unnamed_pro- tein_product_[H.sapiens] | −1.0 | 1.0 | −1.1 | 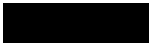 |
| AI553232_ribosomal_protein_S6 | 1.2 | 1.5 | 1.0 | 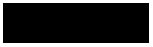 |
| AA210560_Mus_musculus,_Simi- lar_to_RIKEN_cDNA_1500041N16_gene,_cl one_MGC:12066,_mRNA_complete_cds | 1.2 | 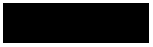 | −1.0 | 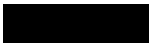 |
| AI037649_transition_protein_1 | −1.1 | 1.2 | −1.2 | 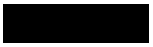 |
| AA717167_cytotoxic_T_lymphocyte-associated_protein_2_alpha | 1.4 | −1.8 | −1.3 | 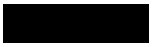 |
| AI553356_ESTs,_Moderately_similar_to_T00075_hypo- thetical_protein_KIAA0461_[H.sapiens] | 1.1 | 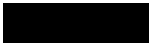 | 1.0 | 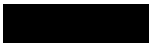 |
| AA710379_neuropilin | −1.08003+ | 1.2 | 1.3 | 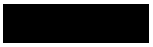 |
| AI463731_ESTs | 1.2 | 1.5 | −1.4 | 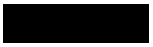 |
| AA710200_ESTs | 1.2 | 1.4 | 1.01548+ | 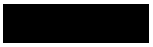 |
| AI464600_ESTs | 1.2 | 1.0 | −1.5 | 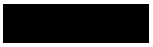 |
| AI643201_Mus_musculus_TOB3_mRNA,_complete_cds | 1.3 | 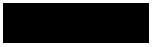 | 1.1 | 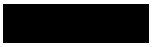 |
| AA445667_RIKEN_cDNA_4632401C08_gene | 1.3 | −1.2 | −2.0 | 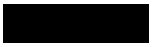 |
| AA498760_xyntaxin_binding_protein_1 | −1.2 | 1.2 | 1.3 | 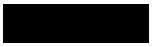 |
| AI613926_twist_gene_homolog,_(Drosophila) | −1.1 | 1.1 | −1.17581* | 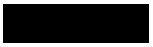 |
| AI549661_RIKEN_cDNA_2410044K02_gene | 1.2 | 1.5 | −1.4 | 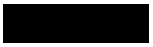 |
| AA269742_RIKEN_cDNA_1110065A22_gene | 1.1 | −1.0 | −1.1 | 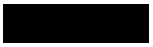 |
| AI594144_DNA_segment,_Chr_3,_Brigham_&_Women's_Ge- netics_0878_expressed | 1.1 | 13 | −1.2 | 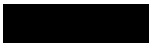 |
| AI607675_Mus_musculus,_clone_MGC:7865,_mRNA,_com- plete_cds | 1.3 | 1.3 | 1.0 | 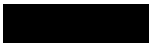 |
| AA230591_myosin,_heavy_polypeptide_3,_skele- tal_muscle,_embryonic | 1.4 | 1.5 | −1.2 | 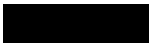 |
| AA671609_core_1_UDP-galactose:N-acetylgalactosamine- alpha-R_beta_1,3-galactosyltransferase | 1.4 | 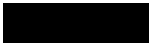 | −1.3 | 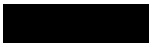 |
| AA450831_BCL2/adenovirus_E1B_19_kDa- interacting_protein_1,_NIP2 | 1.2 | 1.2 | 1.1 | 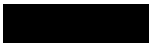 |
| AA638776_matrix_metalloproteinase_12 | 1.26422+ | 1.5439+ | −1.11429+ | 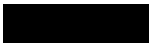 |
| AA110551_phospholipid_scramblase_2 | 1.1 | −1.1 | −1.1 | 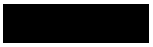 |
| AA833196_RIKEN_cDNA_4932431F02_gene | 1.4 | 1.5 | −1.2 | 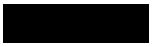 |
| AI550596_stearoyl-Coenzyme_A_desaturase_2 | 1.4 | 1.2 | −1.3 | 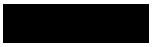 |
| AA655910_mast_cell_protease_2 | 1.12172+ | −1.2816+ | 1.09121+ | 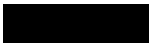 |
| AA412921_RIKEN_cDNA_4921514I20_gene | 1.4 | 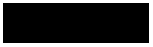 | 1.0 | 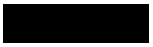 |
| AI552048_ESTs | −1.04537+ | 1.2052+ | −1.0 | 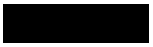 |
| AA265101_RIKEN_cDNA_1600021C16_gene | 1.4 | 1.2 | −1.1 | 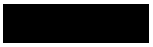 |
| AA688867_ESTs,_Weakly_similar_to_Inv_[M.musculus] | 1.2 | 1.4 | −1.35747+ | 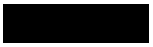 |
| AA516997_kinase_suppressor_of_ras | 1.1 | 1.2 | −1.4 | 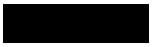 |
| AA254235_complement_receptor_2 | 1.1 | 1.5 | −1.4 | 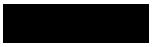 |
| AA450452_RIKEN_cDNA_8430430L24_gene | 1.2 | 1.5 | −1.2 | 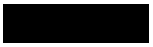 |
| AA596430_galactose-1-phosphate_uridyl_transferase | 1.3 | 1.5 | −1.0 | 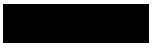 |
| AA023720_ESTs | 1.2 | 1.5 | −1.1 | 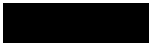 |
| AA267824_Public_domain_EST | 1.3 | 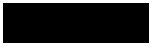 | −1.1 | 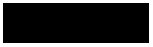 |
| AA607043_RIKEN_cDNA_2400007G07_gene | 1.1 | 1.4 | −1.1 | 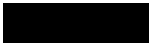 |
| AA273401_Public_domain_EST | 1.5 | 1.3 | −1.0 | 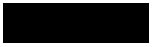 |
| AI466825_RIKEN_cDNA_A330103B21_gene | 1.3 | 1.2 | −1.2 | 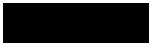 |
| AI644723_ESTs,_Weakly_similar_to_JH0494_alpha-1- antichymotrypsin-like_protein_EB22/4_-_mouse_[M.musculus] | 1.2 | 1.4 | 1.1 | 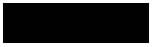 |
| AA469724_WW_domain_binding_protein_5 | 1.3 | 1.4 | −1.1 | 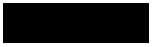 |
| AA474374_RIKEN_cDNA_5731418C10_gene | 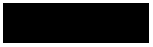 | 1.5 | 1.0 | 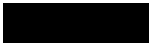 |
| AA450453_Mus_musculus,_Similar_to_hypothetical_pro- tein,_clone_MGC:7581,_mRNA,_complete_cds | 1.4 | 1.3 | −1.2 | 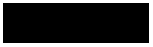 |
| AA792297_procollagen,_type_V,_alpha_1 | −1.2 | 1.0 | −1.15089* | 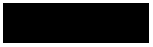 |
| AA105367_period_homolog_3_(Drosophila) | 1.2 | 1.1 | −1.1 | 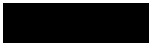 |
| AA466199_RIKEN_cDNA_2410055N2_gene | −1.04614+ | 1.41714+ | −1.26081+ | 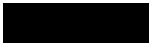 |
| AA823475_ESTs | −1.22121+ | 1.1 | −1.7 | 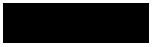 |
| AA638198_gap_junction_membrane_channel_protein_beta_3 | 1.2 | 1.5 | −1.1 | 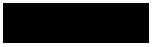 |
| AA529248_Mus_musculus,_Similar_to_phosphoprotein_regu- lated_by_mitogenic_pathways,_clone_MGC:11752,_mRNA,_com- plete_cds | 1.3 | 1.4 | −1.1 | 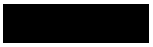 |
| AA066256_Public_domain_EST | 1.1 | 1.2 | −1.1 | 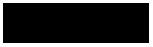 |
| AA470294_DNA_segment,_Chr_2,_ERATO_Doi_435,_expressed | 1.2 | 1.2 | −1.1 | 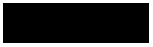 |
| AA250545_ESTs,_Weakly_simi- lar_to_SFRB_SPLICING_FACTOR_ARGININE/SERINE- RICH_11_[H.sapiens] | 1.3 | 1.2 | 1.2 | 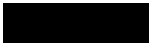 |
| AA869166_defensin_related_cryptdin,_related_sequence_10 | −1.0 | −1.1 | −1.7 | 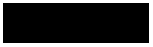 |
| AI608071_RIKEN_cDNA_2900017D14_gene | 1.3 | 1.5 | −1.1 | 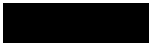 |
| AA155097_ESTs | 1.4 | 1.1 | 1.1 | 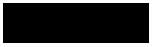 |
| AA518686_ESTs | 1.2 | 1.4 | −1.3 | 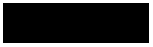 |
| AA616077_ESTs | 1.2 | 1.2 | −1.3 | 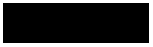 |
| AA204045_Public_domain_EST | 1.4 | 1.4 | 1.1 | 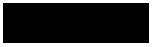 |
| AA212102_ESTs | −1.1 | 1.3 | −1.1 | 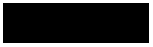 |
| AA058211_ESTs | 1.3 | 1.3 | −1.0 | 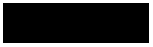 |
| AA870247_mitogene_regulated_protein,_proliferin_3 | −1.0 | 1.0 | −1.3 | 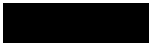 |
| AA562544_RIKEN_cDBA_1110035L05_gene | 1.3 | 1.3 | −1.1 | 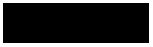 |
| AA210559_ESTs,_Weakly_similar_to_T34029_hypo- thetical_protein_C32F101_-_Caenorhabiditis_elegans_(D.elegans) | −1.1 | 1.3 | 1.0 | 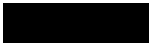 |
| AA684073_ESTs | −1.0 | 1.24978* | −1.3 | 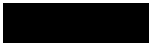 |
| AA497627_intersectin_(SH3_domain_protein_1A) | −1.6 | 1.0 | 1.1 | 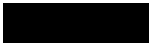 |
| AA717080_ESTs | 1.0 | 1.2 | −1.1 | 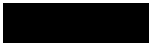 |
| AA244813_calbindin_2 | 1.4 | 1.1 | −1.5 | 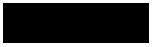 |
| AA265096_ESTs | 1.2 | 1.3 | 1.1 | 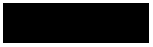 |
| AI464603_RIKEN_cDNA_0710001E13_gene | 1.1 | 1.1 | 1.0 | 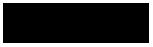 |
| AA254875_ESTs | 1.1 | 1.1 | 1.0 | 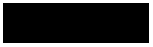 |
| AA646049_interleukin_13 | 1.2 | 1.4 | −1.2 | 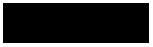 |
| AA290276_ESTs | 1.5 | 1.4 | −1.1 | 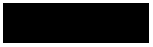 |
| AA211954_RIKEN_cDNA_4930511A05_gene | −1.1 | 1.0 | −1.2 | 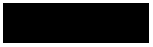 |
| AI530661_RIKEN_cDNA_2810426N06_gene | 15 | 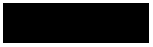 | 1.2 | 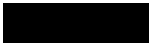 |
| AA571416_RIKEN_cDNA_1190004M21_gene | 1.1 | 1.4 | −1.2 | 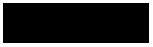 |
| AI627071_RIKEN_cDNA_2310058A11_gene | 1.3 | 1.4 | −1.3 | 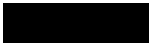 |
| AI597531_gap_junction_membrane_channel_protein_beta_2 | 1.2 | 1.4 | −1.1 | 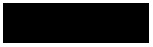 |
| AA591707_DNA_polymerase_epsilon,_subunit_2 | −1.1 | 1.1 | 1.05228* | 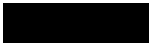 |
| AA473562_ESTs,_Moderately_similar_to_alias_DLC1_[H.sapiens] | 1.3 | 1.5 | 1.2 | 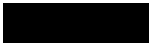 |
| AA220774_ESTs | 1.2 | 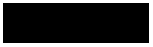 | −1.0 | 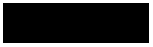 |
| AI117905_RIKEN_cDNA_6530406M24_gene | −1.6 | −1.1 | 1.5 | 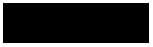 |
| AA395924_ESTs | 1.3 | 1.4 | −1.1 | 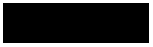 |
| AA869181_defensin_related_cryptdin_6 | −1.8 | −1.7 | −1.5 | 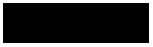 |
| AI604192_RIKEN_cDNA_4632433K11_gene | 1.2 | 1.4 | −1.1 | 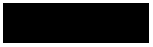 |
| AI550569_ESTs | 1.2 | 1.2 | −1.2 | 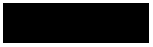 |
| AA473386_ESTs | 1.2 | 1.5 | −1.2 | 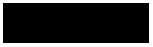 |
| AA591510_homeo_box_B1 | −1.1 | 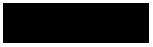 | −1.4 | 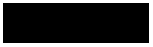 |
| AI587821_solute_carrier_family_22_(organic_cation_trans- porter),_member_2 | 1.4 | 1.1 | −1.3 | 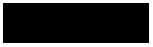 |
| AI553220_ESTs | 1.47814* | −1.0 | 1.3 | 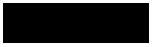 |
| [symbol] | 1.2 | 1.3 | 1.1 | 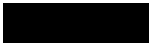 |
| AA067264_kinectin_1 | −1.1 | −1.0 | −1.3 | 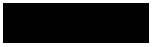 |
| AA266331_ESTs | 1.15878+ | 1.2 | −1.1 | 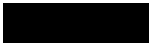 |
| AA596525_chromodomain_helicase_DNA_binding_protein_1 | −1.4 | −1.07406+ | 1.0 | 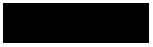 |
| AA209628_ESTs,_Highly_similar_to_T08741_hypo- thetical_protein_DKFZp586G0518.1_[H.sapiens] | 1.1 | −1.1 | −1.0 | 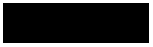 |
| AA259281_LIM_homeobox_protein_2 | 1.4 | 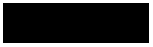 | −1.1 | 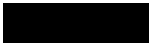 |
| AI549635_ESTs | 1.1 | 1.1 | −1.2 | 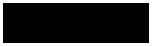 |
| AA268913_flavin_containing_monooxygenase_5 | 1.2 | 1.2 | −1.13647+ | 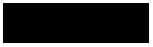 |
| AA547242_undifferentiated_embryonic_cell_transcription_factor_1 | −1.5 | 1.5 | 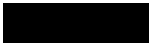 | 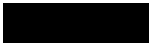 |
| AA060731_nuclear_factor,_interleukin_3,_regulated | 1.4 | 1.2 | −1.1 | 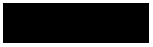 |
| AI596550_leukemia/lymphoma_related_factor | 1.2 | 1.4 | 1.2 | 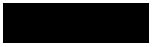 |
| AA822679_hematopoietic_cell_signal_transducer | −1.0 | 1.1 | −1.2 | 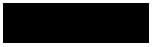 |
| AA517193_unc119_homolog_(C._elegans) | 1.0 | 1.3 | 1.2 | 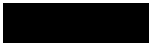 |
| AA691755_RIKEN_cDNA_1110068E08_gene | 1.2 | 1.3 | 1.15501+* | 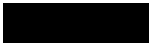 |
| AA739464_killer_cell_lectin- like_receptor_subfamily_A,_member_9 | 1.4 | 1.2 | −1.1 | 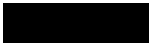 |
| AA413026_Public_domain_EST | −1.0 | 1.1 | −1.4 | 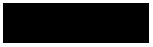 |
| AA510659_ESTs | 1.3 | 1.3 | 1.0 | 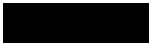 |
| AA798385_ESTs | −1.1 | 1.2 | −1.1 | 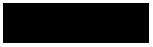 |
| AA438040_Public_domain_EST | −1.1 | 1.1 | −1.1 | 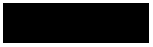 |
| AA474160_ESTs | 1.2 | 1.2 | −1.1 | 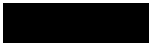 |
| AI614679_ESTs | 1.2 | 1.2 | −1.1 | 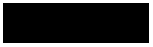 |
| AI450469_ESTs | 1.3 | 1.4 | −1.1 | 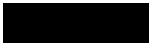 |
| AI551472_ESTs | −1.6 | 1.02324* | −1.0 | 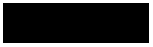 |
| AI594129_Mus_musculus,_clone_IMAGE:3494411_mRNA,_par- tial_cds | 1.1 | 1.2 | −1.3 | 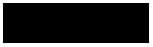 |
| AI390609_ESTs | 1.3 | 1.1881* | −1.0 | 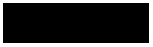 |
| AA277192_RIKEN_cDNA_2900075A18_gene | 1.0 | 1.5 | −1.5 | 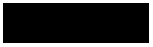 |
| AI604684_RIKEN_cDNA_4631422C13_gene | 1.2 | 1.3 | −1.1 | 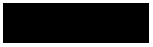 |
| AI789680_ESTs | 1.0429+ | 1.0 | 1.2 | 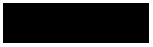 |
| AA270832_ESTs | 1.3 | 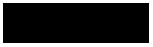 | −1.3 | 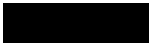 |
| AA268472_RIKEN_cDNA_2410003C20_gene | 1.1 | 1.3 | −1.1 | 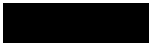 |
| AA438136_nucler_localization_signal_protein_absent_in_velo- cardio-facial_patients | 1.2 | 1.5 | 1.2 | 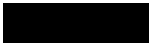 |
| AI592746_ESTs | −1.0 | 1.2 | −1.1 | 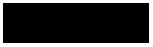 |
| AA259871_hypothetical_protein,_MNCb-4414 | −1.0 | −1.0 | −1.2 | 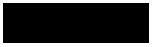 |
| AA710758_Mus_musculus_mRNA_b,b-carotene- 9′,10′-dioxygenase_(b-diox-II_gene) | 1.5 | 1.5 | 1.0 | 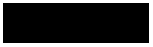 |
| AI464635_ESTs | 1.0 | 1.2 | −1.5 | 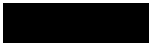 |
| AA895221_ESTs | 1.1 | 1.1 | 1.0 | 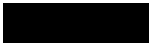 |
| AI551957_RIKEN_cDNA_1810059J10_gene | 1.3 | 1.4 | −1.2 | 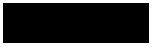 |
| AI614440_ESTs,_Moderately_similar_to_xylulokinase_[H.sapiens] | 1.5 | 1.0 | 1.1 | 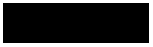 |
| AI464729_ESTs | −1.2 | 1.2 | −1.2 | 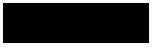 |
| AI614454_reduced_in_osteosclerosis_transporter | 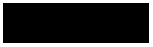 | −1.1 | −1.8 | 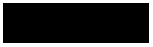 |
| AA185588_ESTs | 1.21114+ | 1.1 | −1.2 | 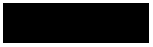 |
| AA690434_RIKEN_cDNA_2010305L05_gene | 1.4 | 1.5 | −1.2 | 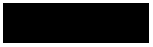 |
| AA615196_ESTs | −1.09609+ | 1.16002+ | −1.07077+ | 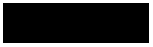 |
| AA068280_ESTs,_Weakly_similar_to_type_III_col- lagen_[M.musculus] | −1.3 | −1.1 | 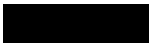 | 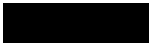 |
| AA105069_ESTs | 1.1 | 1.1 | −1.2 | 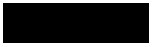 |
| AA104537_c-fos_induce_growth_factor | 1.1 | 1.5 | −1.3 | 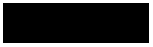 |
| AI323038_cathelin-like_protein | −1.8 | 1.3 | −1.1 | 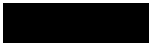 |
| AI645164_RIKEN_cDNA_2610019N06_gene | 1.1 | 1.5 | −1.4 | 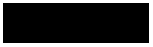 |
| AI326575_glucose-6-phosphatase,_catalytic | 1.3 | −1.6 | 1.4 | 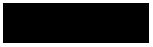 |
| AI593604_ESTs | 1.2 | 1.2 | −1.2 | 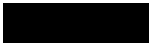 |

STOMACH

| 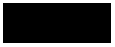 | int | col | brain | lung |
|---|---|---|---|---|
| Description | PL-3B_BDEnor | PL-5B_BDEnor | PL-1B_BDEnor | PL-7B_BDEnor |
| 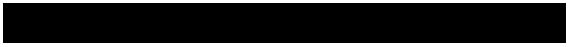 | 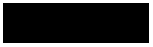 | −1.01549+˜ | 1.1 | 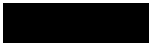 |
| 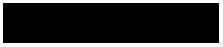 | 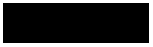 | −1.01971+˜ | 1.3 | 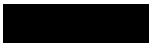 |
| 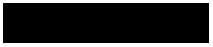 | 1.5 | −1.02494+˜ | 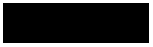 | 1.5 |
| 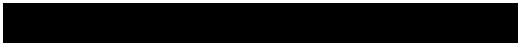 | 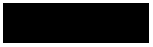 | 1.2437+˜ | 1.4 | 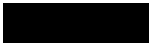 |
| 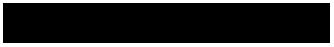 | 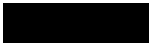 | 1.31548+˜ | 1.3 | 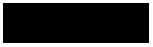 |
| 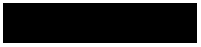 | 1.4283* | −1.60688+˜ | 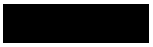 | 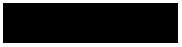 |
| 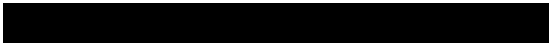 | 1.17207+ | −1.33509+˜ | 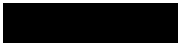 | 1.18149+ |
| 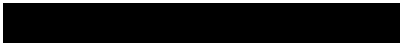 | 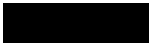 | −1.07432+˜ | 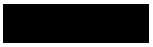 | 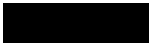 |
| 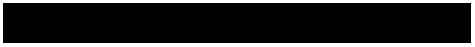 | 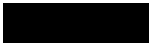 | 1.10261+˜ | 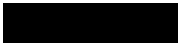 | 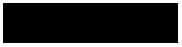 |
| 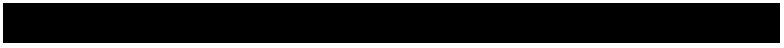 | 1.5 | −1.1 | 1.2 | 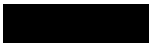 |
| 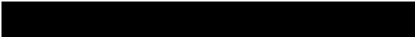 | 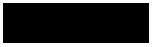 | −1.23958+˜ | 1.1 | 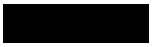 |
| 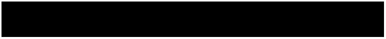 | 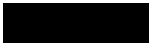 | 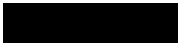 | 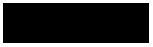 | 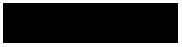 |
| 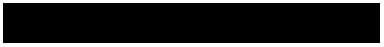 | 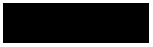 | −1.28917+˜ | 1.4 | 1.4 |
| 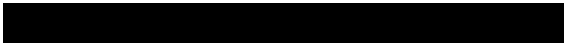 | 1.5 | −1.20826˜ | 1.4 | 1.5 |
| 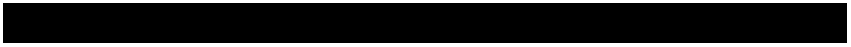 | −2.0 | −2.4 | −1.1 | −1.4 |
| 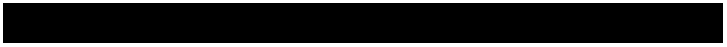 | 1.1 | 1.02178+˜ | 1.2 | 1.3 |
| 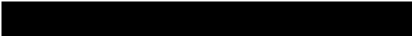 | −1.1 | −1.28021+˜ | 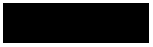 | 1.0 |
| 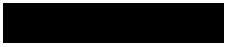 | 1.5 | −1.12264+˜ | 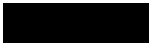 | 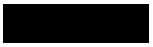 |
| 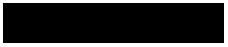 | 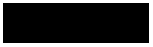 | 1.17361+˜ | 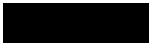 | 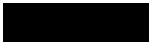 |
| 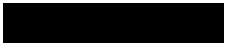 | 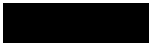 | 1.30792+˜ | 1.3 | 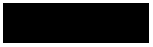 |
| 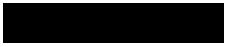 | 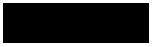 | −1.27077˜ | 1.1 | 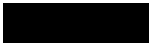 |
| 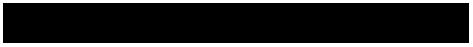 | 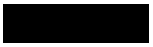 | 1.12103+˜ | −1.0 | 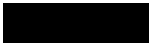 |
| 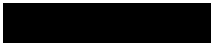 | 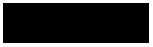 | 1.00205+˜ | 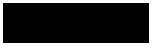 | 1.3 |
| 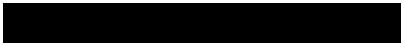 | 1.1 | 1.06724+˜ | 1.4 | 1.3 |
| 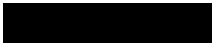 | 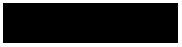 | −1.08065+˜ | 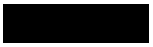 | 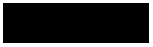 |
| 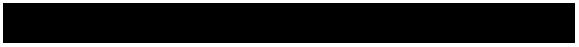 | 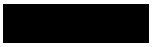 | 1.22798˜ | 1.5 | 1.5 |
| 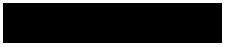 | 1.5 | 1.2483+˜ | 1.2 | 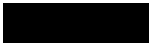 |
| 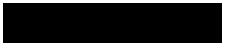 | 1.5 | −1.10185+˜ | 1.2 | 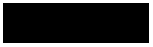 |
| 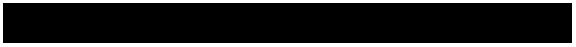 | 1.36143* | −1.3 | 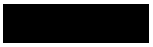 | 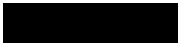 |
| 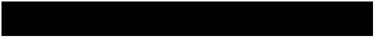 | 1.3 | 1.05496˜ | 1.4 | 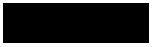 |
| 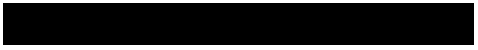 | 1.3 | 1.3 | 1.5 | 1.4 |
| 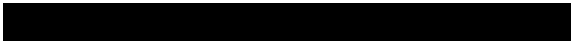 | 1.2 | −1.3 | 1.3 | 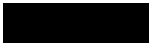 |
| 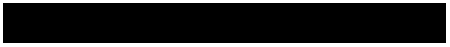 | 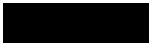 | 1.1254+˜ | 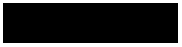 | 1.432+ |
| 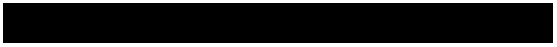 | 1.5 | 1.2409+˜ | 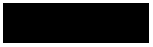 | 1.4 |
| 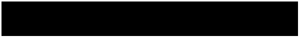 | 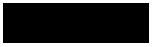 | 1.35346+˜ | 1.5 | 1.4 |
| 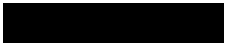 | 1.48785+ | −1.28489+˜ | 1.45708+ | 1.46404+ |
| 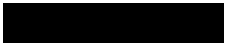 | 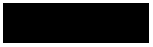 | −1.30902+˜ | 1.4 | 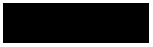 |
| 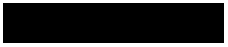 | 1.5 | −1.0 | 1.4 | 1.5 |
| 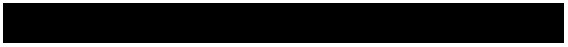 | 1.4 | 1.0667˜ | 1.4 | 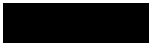 |
| AA638805_Public_domain_EST | 1.5 | −1.12759+˜ | 1.4 | 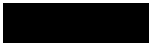 |
| AI550977_ESTs | 1.3 | −1.10355+˜ | 1.5 | 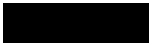 |
| AA683789_sirtuin_1_((silent_mating_type_information_regu- lation_2,_homolog)_1_(S._cerevisiae) | 1.5 | −1.22848+˜ | 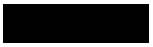 | 1.2 |
| AI549694_ESTs,_Highly_similar_to_P300_HUMAN_E1A- | 1.3 | 1.15359+˜ | −1.1 | 1.4 |
| ASSOCIATED_PROTEIN_P300_[H.sapiens] | | | | |
| AI550200_SEC61,_alpha_subunit_2_(S._cerevisiae) | 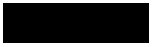 | 1.28243+˜ | 1.2 | 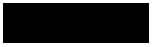 |
| AI645426_diaphanous_homolog_3_(Drosophila) | 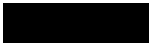 | 1.01107+˜ | 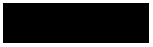 | 1.5 |
| AA231099_necdin | 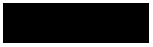 | 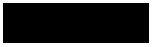 | 1.0 | −2.0 |
| AA530026_ESTs | 1.43296+ | −1.18503+˜ | 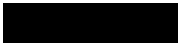 | 1.35485+ |
| AI552219_ESTs | 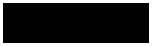 | −1.26743+˜ | 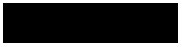 | 1.53405+ |
| AA517691_ESTs | 1.32145* | −1.2 | 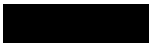 | 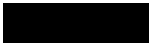 |
| AA517337_WASP_family_1 | 1.4 | 1.40453+˜ | 1.5 | 1.1 |
| AA543950_RIKEN_cDNA_2900073G15_gene | 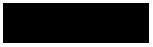 | −1.09881˜ | −1.1 | 1.5 |
| AA617362_low_density_lipoprotein_receptor-related_protein_2 | 1.4 | −1.17835+˜ | 1.53081+ | 1.1 |
| AA473684_RIKEN_cDNA_C030002O17_gene | 1.37779+ | −1.21258+˜ | 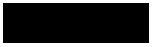 | 1.50959+ |
| AA265337_deoxyguanosine_kinase | 1.4 | −1.04267˜ | 1.1 | 1.4 |
| AA588981_Mus_musculus_brain_cDNA,_clone_MNCb-1820 | 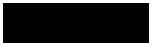 | 1.12888˜ | 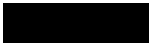 | 1.3 |
| AI325781_ESTs,_Weakly_similar_to_S37771_ankyrin,_erythro- | 1.5 | 1.11463+˜ | 1.5 | 1.5 |
| cyte_-_mouse_[M.musculus] | | | | |
| AI549656_RIKEN_cDNA_3632413F13_gene | 1.4 | −1.1 | 1.1 | 1.5 |
| AI549666_ESTs | 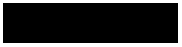 | 1.52793+˜ | 1.07014+ | 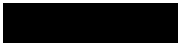 |
| AI425572_ESTs,_Highly_similar_to_zyginl_[R.norvegicus] | 1.2 | 1.10985+˜ | 1.3 | 1.2 |
| AA210502_Public_domain_EST | 1.5 | 1.05245˜ | 1.17392+ | 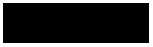 |
| AI509664_RIKEN_cDNA_2410174K12_gene | 1.1 | −1.19466+˜ | 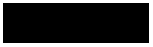 | 1.0 |
| AI587986_ESTs | 1.4 | 1.2339+˜ | 1.33244+ | 1.2 |
| AA606582_Public_domain_EST | 1.4 | 1.08581+˜ | 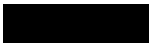 | 1.5 |
| AI551955_RIKEN_cDNA_6330415E02_gene | 1.4 | 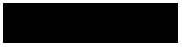 | −1.2 | 1.2 |
| AA823452_ESTs | 1.54682+ | 1.02451+˜ | 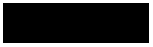 | 1.31484+ |
| AW210270_ESTs,_Weakly_similar_to_T17254_hypo- | 1.3 | −1.1 | 1.4 | 1.4 |
| thetical_protein_KKFZp586O1022.1_[H.sapien] | | | | |
| AA412912_ESTs | 1.5 | −1.07075+˜ | 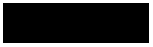 | 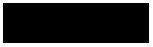 |
| AA068624_hydroxyacid_oxidase_(glycolate_oxidase)_3 | 1.32102* | −2.4 | 1.2 | 1.4 |
| AA096985_chtochrome_P450,_2j5 | 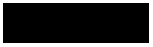 | 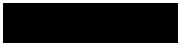 | 1.4 | −1.0 |
| AA709576_ESTs | 1.5 | −1.02969+˜ | 1.5 | 1.33339+ |
| AA611551_Mest-linked_imprinted_transcript_1 | 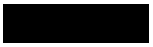 | −1.14544+˜ | 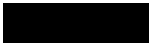 | 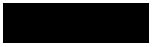 |
| AA000370_RIKEN_cDNA_4933438K12_gene | 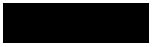 | 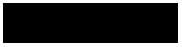 | 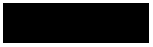 | 1.4 |
| AA153231_Public_domain_EST | 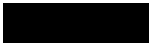 | −1.58578*˜ | 1.3 | 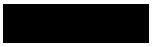 |
| AA288247_ESTs,_Weakly_similar_to_MRP5_MOUSE_MULTI- | 1.0 | 1.11973+˜ | −1.5 | 1+ |
| DRUG_RESISTANCE-ASSOCIATED_PROTEIn_5_[M.musculus] | | | | |
| AA709688_ESTs | 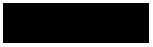 | 1.15298+˜ | 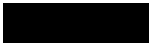 | 1.4 |
| AA615213_ESTs,_Moderately_similar_to_unnamed_pro- | 1.4 | 1.46574+˜ | 1.14599+ | 1.4 |
| tein_product_[H.sapiens] | | | | |
| AI553232_ribosomal_protein_S6 | 1.2 | −1.20582+˜ | 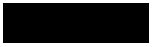 | 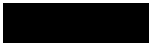 |
| AA210560_Mus_musculus,_Simi- lar_to_RIKEN_cDNA_1500041N16_gene,_cl one_MGC:12066,_mRNA_complete_cds | 1.3 | −1.17168+˜ | 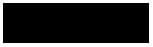 | 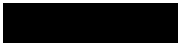 |
| AI037649_transition_protein_1 | 1.4 | −1.3 | −1.4 | 1.5 |
| AA717167_cytotoxic_T_lymphocyte-associated_protein_2_alpha | 1.4 | 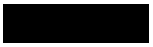 | −1.8 | −1.1 |
| AI553356_ESTs,_Moderately_similar_to_T00075_hypo- thetical_protein_KIAA0461_[H.sapiens] | 1.5 | 1.06805+˜ | 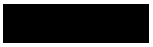 | 1.4 |
| AA710379_neuropilin | 1.4 | −1.63768+˜ | −1.1 | 1.1 |
| AI463731_ESTs | 1.3 | −1.00785+˜ | 1.5 | 1.5 |
| AA710200_ESTs | 1.2 | −1.28921+˜ | 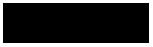 | 1.2 |
| AI464600_ESTs | 1.1 | −1.07437+˜ | 1.4 | 1.4 |
| AI643201_Mus_musculus_TOB3_mRNA,_complete_cds | 1.5 | 1.20006+˜ | 1.25034+ | 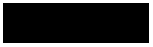 |
| AA445667_RIKEN_cDNA_4632401C08_gene | −1.0 | 1.1 | 1.3 | −1.2 |
| AA498760_xyntaxin_binding_protein_1 | 1.4 | 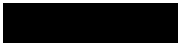 | 1.3 | −1.2 |
| AI613926_twist_gene_homolog,_(Drosophila) | 1.4 | 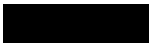 | −1.2 | −1.3 |
| AI549661_RIKEN_cDNA_2410044K02_gene | 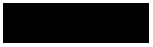 | 1.52602+˜ | 1.3 | 1.4 |
| AA269742_RIKEN_cDNA_1110065A22_gene | 1.3 | 1.06169+˜ | 1.2 | 1.3 |
| AI594144_DNA_segment,_Chr_3,_Brigham_&_Women's_Ge- | 1.4 | −1.17655˜ | 1.1 | 1.5 |
| netics_0878_expressed | | | | |
| AI607675_Mus_musculus,_clone_MGC:7865,_mRNA,_com- | 1.1 | −1.45989˜ | 1.5 | 1.5 |
| plete_cds | | | | |
| AA230591_myosin,_heavy_polypeptide_3,_skele- tal_muscle,_embryonic | 1.3 | 1.16035+˜ | 1.4 | 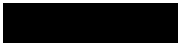 |
| AA671609_core_1_UDP-galactose:N-acetylgalactosamine- alpha-R_beta_1,3-galactosyltransferase | 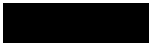 | 1.14962+˜ | 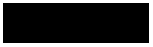 | 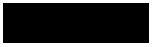 |
| AA450831_BCL2/adenovirus_E1B_19_kDa- interacting_protein_1,_NIP2 | 1.4 | 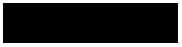 | 1.3 | 1.1 |
| AA638776_matrix_metalloproteinase_12 | 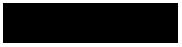 | 1.01168+˜ | 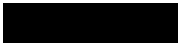 | 1.29835+ |
| AA110551_phospholipid_scramblase_2 | 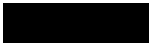 | −1.1 | −1.4 | 1.2 |
| AA833196_RIKEN_cDNA_4932431F02_gene | 1.4 | −1.0 | 1.4 | 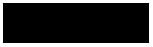 |
| AI550596_stearoyl-Coenzyme_A_desaturase_2 | 1.4 | −1.5 | 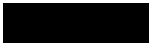 | 1.5 |
| AA655910_mast_cell_protease_2 | 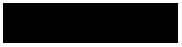 | 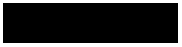 | 1.21826+ | 1.21528+ |
| AA412921_RIKEN_cDNA_4921514I20_gene | 1.4 | −1.23027˜ | 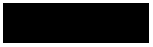 | 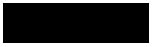 |
| AI552048_ESTs | 1.39436+ | 1.18278+˜ | −1.03946+ | 1.19454+ |
| AA265101_RIKEN_cDNA_1600021C16_gene | 1.1 | 1.03533+˜ | −1.3 | 1.3 |
| AA688867_ESTs,_Weakly_similar_to_Inv_[M.musculus] | 1.5 | 1.18935+˜ | 1.4 | 1.3 |
| AA516997_kinase_suppressor_of_ras | 1.4 | 1.17949+˜ | 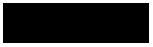 | 1.2 |
| AA254235_complement_receptor_2 | 1.4 | −1.02477+˜ | 1.5 | 1.4 |
| AA450452_RIKEN_cDNA_8430430L24_gene | 1.5 | 1.2900+˜ | 1.4 | 1.3 |
| AA596430_galactose-1-phosphate_uridyl_transferase | 1.4 | −1.08755+˜ | 1.4 | 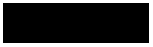 |
| AA023720_ESTs | 1.2 | 1.0728+˜ | 1.5 | 1.5 |
| AA267824_Public_domain_EST | 1.32119* | −1.13095+˜ | 1.3 | 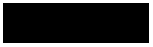 |
| AA607043_RIKEN_cDNA_2400007G07_gene | 1.5 | 1.00337+˜ | 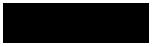 | 1.3 |
| AA273401_Public_domain_EST | 1.40823* | −1.0 | 1.3 | 1.5 |
| AI466825_RIKEN_cDNA_A330103B21_gene | 1.4 | 1.1 | 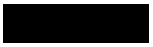 | 1.4 |
| AI644723_ESTs,_Weakly_similar_to_JH0494_alpha-1- | 1.2 | −1.11434˜ | 1.1 | 1.4 |
| antichymotrypsin-like_protein_EB22/4_-_mouse_[M.musculus] | | | | |
| AA469724_WW_domain_binding_protein_5 | 1.5 | 1.0 | 1.1 | 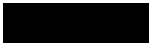 |
| AA474374_RIKEN_cDNA_5731418C10_gene | 1.3 | −1.07742˜ | 1.5 | 1.4 |
| AA450453_Mus_musculus,_Similar_to_hypothetical_pro- tein,_clone_MGC:7581,_mRNA,_complete_cds | 1.4 | 1.02813˜ | 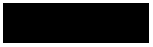 | 1.3 |
| AA792297_procollagen,_type_V,_alpha_1 | 1.5 | 1.4 | −1.4 | 1.0 |
| AA105367_period_homolog_3_(Drosophila) | 1.2 | 1.11211+˜ | −1.1 | 1.3 |
| AA466199_RIKEN_cDNA_2410055N2_gene | 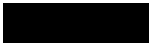 | −1.0338+˜ | 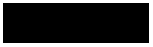 | 1.2862+ |
| AA823475_ESTs | 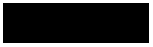 | −1.43639+˜ | 1.3 | 1.5 |
| AA638198_gap_junction_membrane_channel_protein_beta_3 | 1.2 | −1.31763˜ | 1.5 | 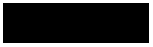 |
| AA529248_Mus_musculus,_Similar_to_phosphoprotein_regu- | 1.4 | −1.2 | 1.3 | 1.5 |
| lated_by_mitogenic_pathways,_clone_MGC:11752,_mRNA,_com- | | | | |
| plete_cds | | | | |
| AA066256_Public_domain_EST | 1.4 | 1.46053+˜ | 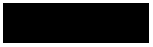 | 1.4 |
| AA470294_DNA_segment,_Chr_2,_ERATO_Doi_435,_expressed | 1.4 | −1.0 | 1.5 | 1.4 |
| AA250545_ESTs,_Weakly_simi- | 1.1 | −1.41089+˜ | 1.5 | 1.5 |
| lar_to_SFRB_SPLICING_FACTOR_ARGININE/SERINE- | | | | |
| RICH_11_[H.sapiens] | | | | |
| AA869166_defensin_related_cryptdin,_related_sequence_10 | 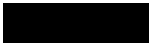 | 1.5 | 1.2 | 1.2 |
| AI608071_RIKEN_cDNA_2900017D14_gene | 1.2 | −1.2 | 1.4 | 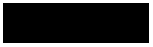 |
| AA155097_ESTs | 1.2 | −1.2 | 1.4 | 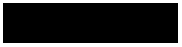 |
| AA518686_ESTs | 1.5 | −1.5 | 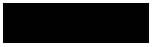 | 1.4 |
| AA616077_ESTs | 1.5 | −1.10029+˜ | 1.2 | 1.5 |
| AA204045_Public_domain_EST | 1.3 | −1.2 | −1.1 | 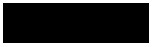 |
| AA212102_ESTs | 1.2 | 1.16314+˜ | 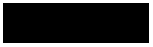 | 1.1 |
| AA058211_ESTs | 1.1 | −1.0 | 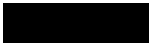 | 1.3 |
| AA870247_mitogene_regulated_protein,_proliferin_3 | 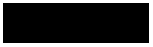 | −1.0 | −1.8 | 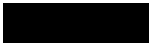 |
| AA562544_RIKEN_cDBA_1110035L05_gene | 1.3 | 1.0 | 1.4 | 1.5 |
| AA210559_ESTs,_Weakly_similar_to_T34029_hypo- thetical_protein_C32F101_-_Caenorhabiditis_elegans_(D.elegans) | 1.3 | −1.00634˜ | 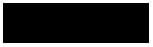 | 1.0 |
| AA684073_ESTs | 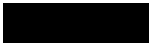 | 1.12054+˜ | 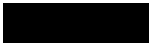 | 1.3 |
| AA497627_intersectin_(SH3_domain_protein_1A) | 1.5 | 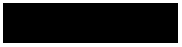 | −1.0 | −1.8 |
| AA717080_ESTs | 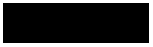 | 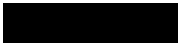 | −1.2 | 1.2 |
| AA244813_calbindin_2 | 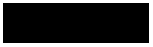 | −1.16196+˜ | 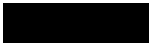 | 1.4 |
| AA265096_ESTs | 1.3 | −1.08802+˜ | 1.5 | 1.5 |
| AI464603_RIKEN_cDNA_0710001E13_gene | 1.4 | −1.08185+˜ | 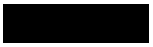 | 1.08953+ |
| AA254875_ESTs | 1.1 | −1.0806+˜ | −1.11044+ | 1.4 |
| AA646049_interleukin_13 | 1.5 | −1.02129+˜ | 1.3 | 1.4 |
| AA290276_ESTs | 1.2 | −1.23451+˜ | 1.0 | 1.5 |
| AA211954_RIKEN_cDNA_4930511A05_gene | 1.2 | 1.1 | 1.3 | 1.1 |
| AI530661_RIKEN_cDNA_2810426N06_gene | 1.2 | −1.40231˜ | 1.10197+ | 1.3 |
| AA571416_RIKEN_cDNA_1190004M21_gene | 1.4 | 1.07884˜ | 1.3 | 1.4 |
| AI627071_RIKEN_cDNA_2310058A11_gene | 1.4 | −1.21372+˜ | −1.1 | 1.5 |
| AI597531_gap_junction_membrane_channel_protein_beta_2 | 1.2 | 1.17509+˜ | 1.3 | 1.3 |
| AA591707_DNA_polymerase_epsilon,_subunit_2 | 1.4 | 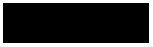 | −1.6 | 1.1 |
| AA473562_ESTs,_Moderately_similar_to_alias_DLC1_[H.sapiens] | 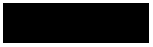 | −1.306+˜ | 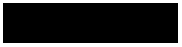 | 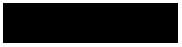 |
| AA220774_ESTs | 1.3 | −1.05803˜ | −1.0 | 1.5 |
| AI117905_RIKEN_cDNA_6530406M24_gene | 1.4 | 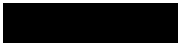 | −1.3 | −1.51858+ |
| AA395924_ESTs | 1.3 | −1.07451˜ | 1.3 | 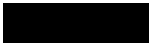 |
| AA869181_defensin_related_cryptdin_6 | 1.5 | 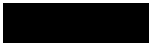 | −1.4 | −1.2 |
| AI604192_RIKEN_cDNA_4632433K11_gene | 1.2 | 1.13766+˜ | 1.2 | 1.2 |
| AI550569_ESTs | 1.2 | −1.18773˜ | −1.2 | 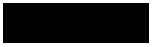 |
| AA473386_ESTs | 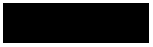 | 1.10149+˜ | 1.0 | 1.5 |
| AA591510_homeo_box_B1 | 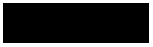 | 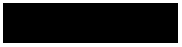 | 1.2 | −1.0 |
| AI587821_solute_carrier_family_22_(organic_cation_trans- | 1.5 | −1.05076˜ | 1.1 | 1.3 |
| porter),_member_2 | | | | |
| AI553220_ESTs | 1.16813* | −1.30242˜ | −1.1 | 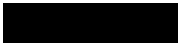 |
| [symbol] | 1.2 | −1.3 | −1.0 | 1.4 |
| AA067264_kinectin_1 | 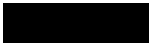 | 1.50139+˜ | 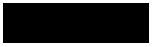 | 1.1 |
| AA266331_ESTs | 1.3 | 1.00375+˜ | 1.1 | 1.4 |
| AA596525_chromodomain_helicase_DNA_binding_protein_1 | 1.1 | 1.34588+˜ | −1.59695+ | 1.0 |
| AA209628_ESTs,_Highly_similar_to_T08741_hypo- | 1.3 | 1.1 | −1.0 | 1.5 |
| thetical_protein_DKFZp586G0518.1_[H.sapiens] | | | | |
| AA259281_LIM_homeobox_protein_2 | 1.2 | −1.2 | 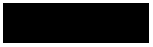 | 1.4 |
| AI549635_ESTs | 1.1 | 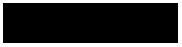 | −1.39628+ | 1.1 |
| AA268913_flavin_containing_monooxygenase_5 | 1.2 | −1.15925+˜ | 1.2 | 1.29569+ |
| AA547242_undifferentiated_embryonic_cell_transcription_factor_1 | 1.4 | 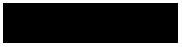 | 1.09749+ | −1.90264+ |
| AA060731_nuclear_factor,_interleukin_3,_regulated | 1.3 | 1.16127+˜ | −1.0 | 1.4 |
| AI596550_leukemia/lymphoma_related_factor | 1.5 | −1.0538+˜ | 1.4 | 1.5 |
| AA822679_hematopoietic_cell_signal_transducer | 1.3 | −1.79549+˜ | 1.2 | 1.2 |
| AA517193_unc119_homolog_(C._elegans) | 1.5 | −1.01587+˜ | 1.0 | −1.0 |
| AA691755_RIKEN_cDNA_1110068E08_gene | 1.3 | −1.29545+˜ | 1.3 | 1.3 |
| AA739464_killer_cell_lectin- | 1.4 | −1.0582* | 1.1 | 1.5 |
| like_receptor_subfamily_A,_member_9 | | | | |
| AA413026_Public_domain_EST | 1.3 | 1.32426+˜ | −1.1 | 1.4 |
| AA510659_ESTs | 1.2 | −1.0885+˜ | 1.5 | 1.5 |
| AA798385_ESTs | 1.2 | −1.03504+˜ | 1.1 | 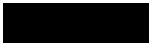 |
| AA438040_Public_domain_EST | 1.5 | 1.38343˜ | −1.1 | 1.2 |
| AA474160_ESTs | 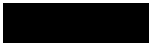 | 1.31659˜ | 1.5 | 1.4 |
| AI614679_ESTs | 1.2 | 1.2 | 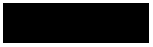 | 1.2 |
| AI450469_ESTs | 1.3 | −1.2 | 1.1 | 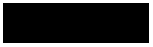 |
| AI551472_ESTs | 1.1 | 1.47107+˜ | −1.65524+ | −1.6 |
| AI594129_Mus_musculus,_clone_IMAGE:3494411_mRNA,_par- tial_cds | 1.1 | −1.16368+˜ | 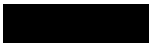 | 1.1 |
| AI390609_ESTs | 1.5 | 1.06588+˜ | 1.5 | 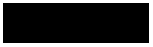 |
| AA277192_RIKEN_cDNA_2900075A18_gene | 1.5 | 1.0011+˜ | 1.4 | 1.3 |
| AI604684_RIKEN_cDNA_4631422C13_gene | 1.3 | 1.17508+˜ | 1.5 | 1.4 |
| AI789680_ESTs | 1.5 | 1.21967+˜ | 1.1 | 1.2 |
| AA270832_ESTs | 1.3 | −1.02764+˜ | 1.2 | 1.5 |
| AA268472_RIKEN_cDNA_2410003C20_gene | 1.3 | −1.17707+˜ | 1.2 | 1.2 |
| AA438136_nucler_localization_signal_protein_absent_in_velo- | 1.3 | −1.04778+˜ | 1.3 | 1.5 |
| cardio-facial_patients | | | | |
| AI592746_ESTs | 1.2 | 1.00816+˜ | 1.03689+ | 1.5 |
| AA259871_hypothetical_protein,_MNCb-4414 | 1.2 | −1.09183+˜ | 1.1 | 1.5 |
| AA710758_Mus_musculus_mRNA_b,b-carotene- | 1.2 | −1.1 | 1.4 | 1.5 |
| 9′,10′-dioxygenase_(b-diox-II_gene) | | | | |
| AI464635_ESTs | 1.5 | −1.27046+˜ | 1.5 | 1.1 |
| AA895221_ESTs | 1.2 | 1.32121˜ | −1.3 | 1.3 |
| AI551957_RIKEN_cDNA_1810059J10_gene | 1.5 | −1.2 | −1.2 | 1.4 |
| AI614440_ESTs,_Moderately_similar_to_xylulokinase_[H.sapiens] | 1.4 | 1.2004˜ | −1.2 | 1.2 |
| AI464729_ESTs | 1.4 | −1.14563+˜ | 1.3 | 1.2 |
| AI614454_reduced_in_osteosclerosis_transporter | 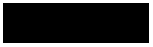 | 1.11125˜ | 1.3 | 1.2 |
| AA185588_ESTs | 1.2 | −1.11004+˜ | −1.15296+ | 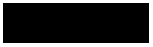 |
| AA690434_RIKEN_cDNA_2010305L05_gene | 1.3 | 1.1 | 1.3 | 1.4 |
| AA615196_ESTs | 1.49956+ | 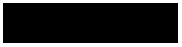 | 1.51513+ | 1.10119+ |
| AA068280_ESTs,_Weakly_similar_to_type_III_col- lagen_[M.musculus] | 1.36706* | 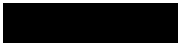 | −1.3 | −1.3 |
| AA105069_ESTs | 1.5 | −1.28705˜ | 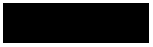 | 1.4 |
| AA104537_c-fos_induce_growth_factor | 1.5 | −1.3 | 1.3 | 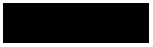 |
| AI323038_cathelin-like_protein | 1.3 | −1.00202+˜ | 1.5 | 1.3 |
| AI645164_RIKEN_cDNA_2610019N06_gene | 1.4 | −1.01691+˜ | 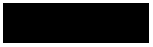 | 1.4 |
| AI326575_glucose-6-phosphatase,_catalytic | 1.4 | 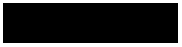 | −2.3 | −1.2 |
| AI593604_ESTs | 1.5 | −1.14396+˜ | 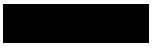 | 1.5 |

STOMACH

| 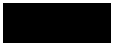 | blad | kidn | pl/ut | mam |
|---|---|---|---|---|
| Description | PL-9B_BDEnor | PL-10B_BDEnor | PL-11B_BDEnor | PL-12B_BDEnor |
| 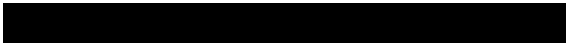 | 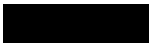 | 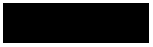 | 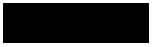 | 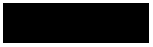 |
| 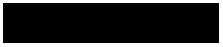 | 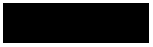 | 1.3 | 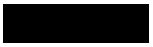 | 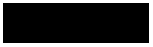 |
| 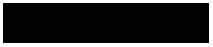 | 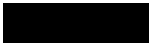 | 1.3 | 1.5 | 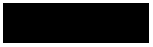 |
| 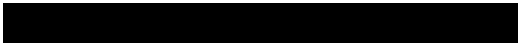 | 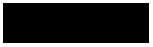 | 1.4 | 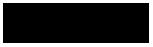 | 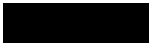 |
| 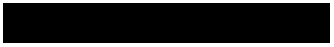 | 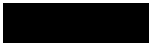 | 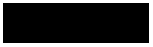 | 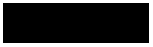 | 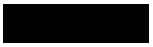 |
| 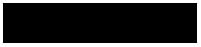 | 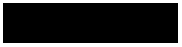 | 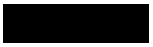 | 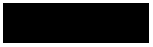 | 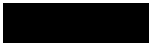 |
| 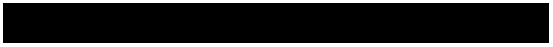 | 1.29226+ | 1.4 | 1.2 | 1.13038+ |
| 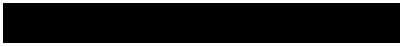 | 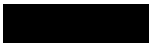 | 1.5 | 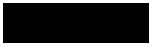 | 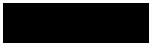 |
| 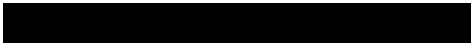 | 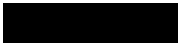 | 1.3 | 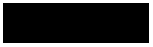 | 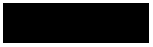 |
| 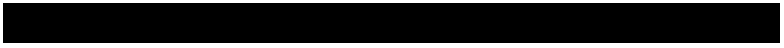 | 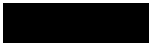 | 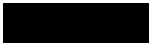 | 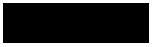 | 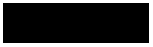 |
| 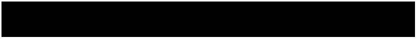 | 1.4 | 1.1 | 1.5 | 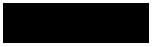 |
| 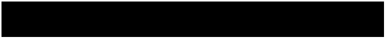 | 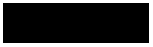 | 1.5 | 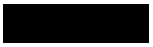 | 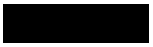 |
| 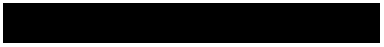 | 1.2 | 1.3 | 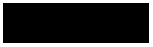 | 1.2 |
| 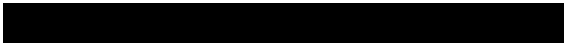 | 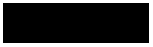 | 1.3 | 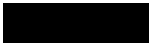 | 1.5 |
| 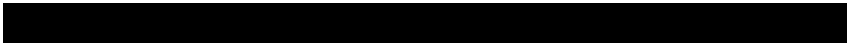 | 1.5 | 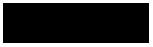 | 1.1 | 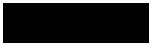 |
| 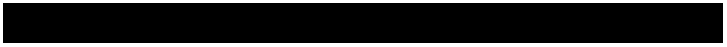 | 1.0 | 1.1 | 1.1 | 1.3 |
| 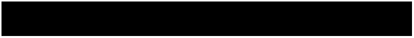 | −1.2 | 1.2 | −1.2 | −1.0 |
| 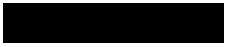 | 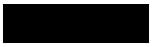 | 1.3 | 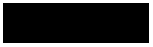 | 1.5 |
| 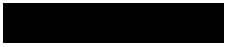 | 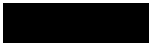 | 1.4 | 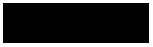 | 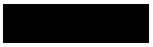 |
| 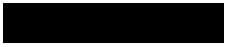 | 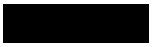 | 1.2 | 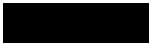 | 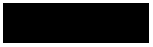 |
| 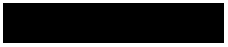 | 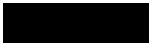 | 1.2 | 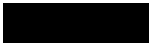 | 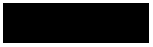 |
| 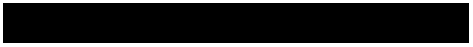 | 1.4 | 1.3 | 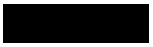 | 1.3 |
| 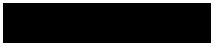 | 1.5 | 1.5 | 1.3 | 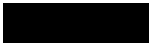 |
| 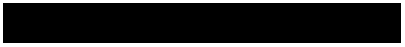 | 1.5 | 1.1 | 1.3 | 1.4 |
| 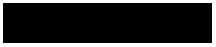 | 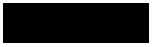 | 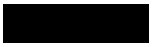 | 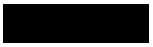 | 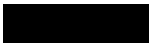 |
| 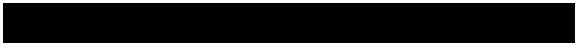 | 1.5 | 1.3 | 1.5 | 1.5 |
| 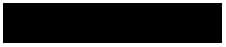 | 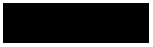 | 1.4 | 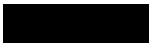 | 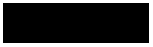 |
| 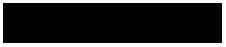 | 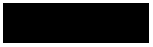 | 1.2 | 1.5 | 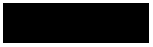 |
| 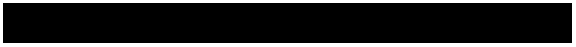 | 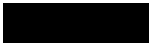 | 1.4 | 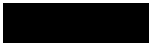 | 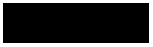 |
| 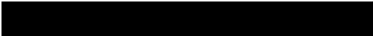 | 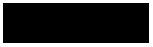 | 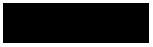 | 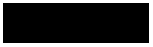 | 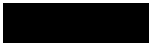 |
| 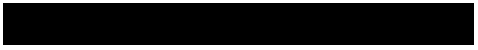 | 1.5 | 1.2 | 1.2 | 1.3 |
| 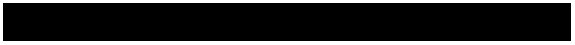 | 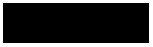 | 1.5 | 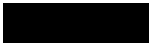 | 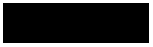 |
| 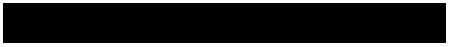 | 1.5 | 1.2 | 1.4 | 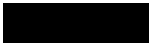 |
| 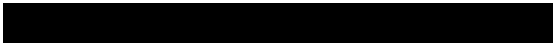 | 1.5 | 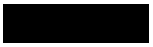 | 1.5 | 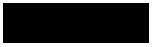 |
| 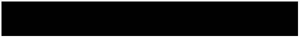 | 1.5 | 1.3 | 1.5 | 1.4 |
| 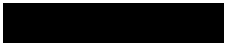 | 1.53133+ | 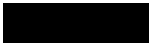 | 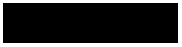 | 1.32862+ |
| 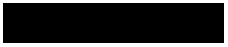 | 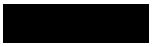 | 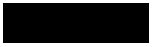 | 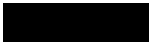 | 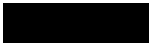 |
| 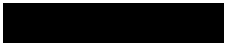 | 1.3 | 1.4 | 1.3 | 1.4 |
| 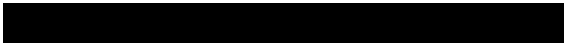 | 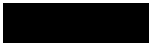 | 1.4 | 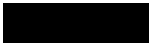 | 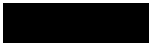 |
| AA638805_Public_domain_EST | 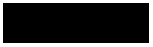 | 1.3 | 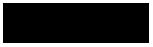 | 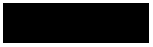 |
| AI550977_ESTs | 1.4 | 1.2 | 1.5 | 1.5 |
| AA683789_sirtuin_1_((silent_mating_type_information_regu- | 1.4 | 1.1 | 1.3 | 1.3 |
| lation_2,_homolog)_1_(S._cerevisiae) | | | | |
| AI549694_ESTs,_Highly_similar_to_P300_HUMAN_E1A- ASSOCIATED_PROTEIN_P300_[H.sapiens] | 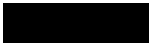 | 1.4 | 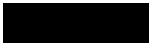 | 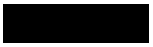 |
| AI550200_SEC61,_alpha_subunit_2_(S._cerevisiae) | 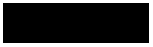 | 1.5 | 1.4 | 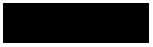 |
| AI645426_diaphanous_homolog_3_(Drosophila) | 1.3 | 1.2 | 1.3 | 1.4 |
| AA231099_necdin | −1.3 | −1.2 | −1.7 | 1.0 |
| AA530026_ESTs | 1.36148+ | 1.3 | 1.5 | 1.1 |
| AI552219_ESTs | 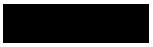 | 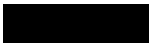 | 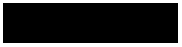 | 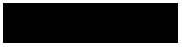 |
| AA517691_ESTs | 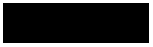 | 1.5 | 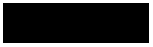 | 1.5 |
| AA517337_WASP_family_1 | 1.4 | 1.2 | 1.2 | 1.3 |
| AA543950_RIKEN_cDNA_2900073G15_gene | 1.3 | 1.2 | 1.5 | 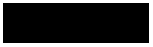 |
| AA617362_low_density_lipoprotein_receptor-related_protein_2 | 1.5 | 1.4 | 1.0 | 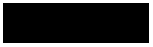 |
| AA473684_RIKEN_cDNA_C030002O17_gene | 1.3697+ | 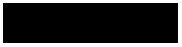 | 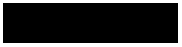 | 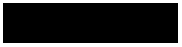 |
| AA265337_deoxyguanosine_kinase | 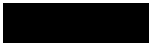 | 1.3 | 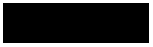 | 1.5 |
| AA588981_Mus_musculus_brain_cDNA,_clone_MNCb-1820 | 1.1 | 1.2 | 1.3 | 1.1 |
| AI325781_ESTs,_Weakly_similar_to_S37771_ankyrin,_erythro- cyte_-_mouse_[M.musculus] | 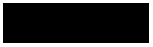 | 1.4 | 1.5 | 1.4 |
| AI549656_RIKEN_cDNA_3632413F13_gene | 1.4 | 1.2 | 1.3 | 1.4 |
| AI549666_ESTs | 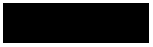 | 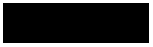 | 1.5 | 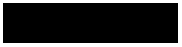 |
| AI425572_ESTs,_Highly_similar_to_zyginl_[R.norvegicus] | 1.3 | −1.1 | 1.2 | 1.3 |
| AA210502_Public_domain_EST | 1.5 | 1.5 | 1.4 | 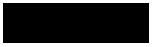 |
| AI509664_RIKEN_cDNA_2410174K12_gene | 1.1 | 1.1 | 1.1 | 1.1 |
| AI587986_ESTs | 1.4 | −1.1 | 1.3 | 1.5 |
| AA606582_Public_domain_EST | 1.3 | 1.3 | 1.5 | 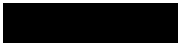 |
| AI551955_RIKEN_cDNA_6330415E02_gene | 1.1 | 1.3 | 1.2 | 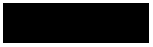 |
| AA823452_ESTs | 1.38275+ | 1.2 | 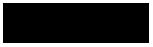 | 1.40132+ |
| AW210270_ESTs,_Weakly_similar_to_T17254_hypo- | 1.4 | 1.27852* | 1.4 | 1.3 |
| thetical_protein_KKFZp586O1022.1_[H.sapien] | | | | |
| AA412912_ESTs | 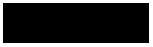 | 1.2 | 1.5 | 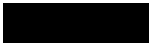 |
| AA068624_hydroxyacid_oxidase_(glycolate_oxidase)_3 | 1.3 | 1.1 | 1.3 | 1.5 |
| AA096985_chtochrome_P450,_2j5 | 1.5 | 1.2 | 1.3 | 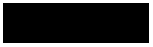 |
| AA709576_ESTs | 1.4 | 1.4 | 1.1 | 1.1 |
| AA611551_Mest-linked_imprinted_transcript_1 | 1.3 | −1.1 | 1.4 | 1.3 |
| AA000370_RIKEN_cDNA_4933438K12_gene | 1.5 | 1.0 | 1.3 | 1.3 |
| AA153231_Public_domain_EST | 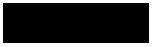 | 1.5 | 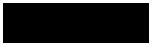 | 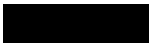 |
| AA288247_ESTs,_Weakly_similar_to_MRP5_MOUSE_MULTI- | 1.0 | −1.1 | 1.3 | 1.4 |
| DRUG_RESISTANCE-ASSOCIATED_PROTEIn_5_[M.musculus] | | | | |
| AA709688_ESTs | 1.4 | 1.2 | 1.2 | 1.2 |
| AA615213_ESTs,_Moderately_similar_to_unnamed_pro- | 1.5 | 1.2 | 1.4 | 1.3 |
| tein_product_[H.sapiens] | | | | |
| AI553232_ribosomal_protein_S6 | 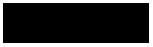 | 1.4 | 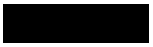 | 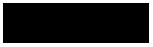 |
| AA210560_Mus_musculus,_Simi- | 1.5 | 1.4 | 1.5 | 1.3 |
| lar_to_RIKEN_cDNA_1500041N16_gene,_cl | | | | |
| one_MGC:12066,_mRNA_complete_cds | | | | |
| AI037649_transition_protein_1 | 1.2 | 1.2 | 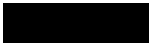 | 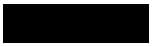 |
| AA717167_cytotoxic_T_lymphocyte-associated_protein_2_alpha | 1.5 | −1.4 | −1.5 | 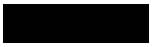 |
| AI553356_ESTs,_Moderately_similar_to_T00075_hypo- thetical_protein_KIAA0461_[H.sapiens] | 1.2 | 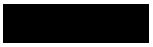 | 1.3 | 1.3 |
| AA710379_neuropilin | 1.2 | −1.1 | 1.3 | 1.4 |
| AI463731_ESTs | 1.4 | 1.4 | 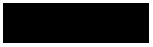 | 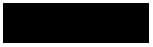 |
| AA710200_ESTs | 1.2 | 1.1 | 1.1 | 1.1 |
| AI464600_ESTs | 1.2 | 1.3 | 1.4 | 1.4 |
| AI643201_Mus_musculus_TOB3_mRNA,_complete_cds | 1.4 | 1.5 | 1.5 | 1.4 |
| AA445667_RIKEN_cDNA_4632401C08_gene | 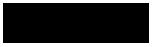 | 1.4 | 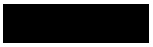 | 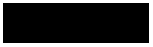 |
| AA498760_xyntaxin_binding_protein_1 | −1.3 | −1.1 | −1.0 | 1.1 |
| AI613926_twist_gene_homolog,_(Drosophila) | 1.1 | 1.0 | −1.2 | −1.0 |
| AI549661_RIKEN_cDNA_2410044K02_gene | 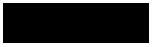 | 1.2 | 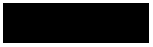 | 1.4 |
| AA269742_RIKEN_cDNA_1110065A22_gene | 1.2 | 1.2 | 1.3 | 1.5 |
| AI594144_DNA_segment,_Chr_3,_Brigham_&_Women's_Ge- | 1.3 | 1.2 | 1.2 | 1.3 |
| netics_0878_expressed | | | | |
| AI607675_Mus_musculus,_clone_MGC:7865,_mRNA,_com- | 1.5 | 1.2 | 1.5 | 1.4 |
| plete_cds | | | | |
| AA230591_myosin,_heavy_polypeptide_3,_skele- tal_muscle,_embryonic | 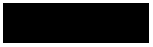 | 1.4 | 1.5 | 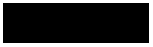 |
| AA671609_core_1_UDP-galactose:N-acetylgalactosamine- alpha-R_beta_1,3-galactosyltransferase | 1.5 | 1.3 | 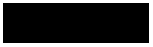 | 1.5 |
| AA450831_BCL2/adenovirus_E1B_19_kDa- | 1.2 | 1.1 | 1.1 | 1.0 |
| interacting_protein_1,_NIP2 | | | | |
| AA638776_matrix_metalloproteinase_12 | 1.4481+ | 1.4 | 1.2 | 1.48751+ |
| AA110551_phospholipid_scramblase_2 | 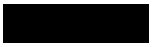 | −1.0 | 1.2 | 1.2 |
| AA833196_RIKEN_cDNA_4932431F02_gene | 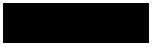 | 1.4 | 1.5 | 1.5 |
| AI550596_stearoyl-Coenzyme_A_desaturase_2 | 1.5 | 1.2 | 1.4 | 1.3 |
| AA655910_mast_cell_protease_2 | 1.0051+ | −1.07124+ | −1.28511+ | −1.1 |
| AA412921_RIKEN_cDNA_4921514I20_gene | 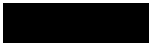 | 1.4 | 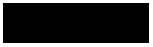 | 1.5 |
| AI552048_ESTs | 1.2 | −1.0 | 1.3 | 1.0 |
| AA265101_RIKEN_cDNA_1600021C16_gene | 1.3 | 1.1 | 1.3 | 1.4 |
| AA688867_ESTs,_Weakly_similar_to_Inv_[M.musculus] | 1.2 | 1.0 | 1.3 | 1.1 |
| AA516997_kinase_suppressor_of_ras | 1.4 | 1.2 | 1.3 | 1.4 |
| AA254235_complement_receptor_2 | 1.2 | 1.1 | 1.4 | 1.3 |
| AA450452_RIKEN_cDNA_8430430L24_gene | 1.5 | 1.1 | 1.4 | 1.4 |
| AA596430_galactose-1-phosphate_uridyl_transferase | 1.4 | 1.2 | 1.4 | 1.1 |
| AA023720_ESTs | 1.3 | 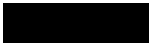 | 1.5 | 1.3 |
| AA267824_Public_domain_EST | 1.5 | 1.3 | 1.5 | 1.5 |
| AA607043_RIKEN_cDNA_2400007G07_gene | 1.4 | 1.4 | 1.3 | 1.3 |
| AA273401_Public_domain_EST | 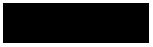 | 1.4 | 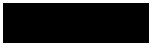 | 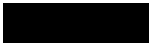 |
| AI466825_RIKEN_cDNA_A330103B21_gene | 1.3 | 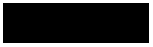 | 1.4 | 1.3 |
| AI644723_ESTs,_Weakly_similar_to_JH0494_alpha-1- antichymotrypsin-like_protein_EB22/4_-_mouse_[M.musculus] | 1.5 | 1.3 | 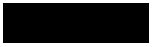 | 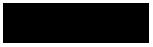 |
| AA469724_WW_domain_binding_protein_5 | 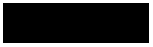 | 1.3 | 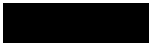 | 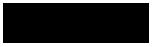 |
| AA474374_RIKEN_cDNA_5731418C10_gene | 1.3 | 1.4 | 1.5 | 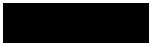 |
| AA450453_Mus_musculus,_Similar_to_hypothetical_pro- tein,_clone_MGC:7581,_mRNA,_complete_cds | 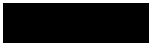 | 1.2 | 1.5 | 1.3 |
| AA792297_procollagen,_type_V,_alpha_1 | 1.2 | 1.1 | −1.1 | −1.3 |
| AA105367_period_homolog_3_(Drosophila) | 1.3 | 1.1 | 1.4 | 1.4 |
| AA466199_RIKEN_cDNA_2410055N2_gene | 1.2 | 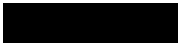 | 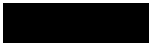 | 1.2 |
| AA823475_ESTs | 1.4 | 1.2 | 1.3 | 1.3 |
| AA638198_gap_junction_membrane_channel_protein_beta_3 | 1.3 | 1.49755+ | 1.4 | 1.3 |
| AA529248_Mus_musculus,_Similar_to_phosphoprotein_regu- | 1.5 | 1.2 | 1.5 | 1.5 |
| lated_by_mitogenic_pathways,_clone_MGC:11752,_mRNA,_com- | | | | |
| plete_cds | | | | |
| AA066256_Public_domain_EST | 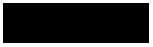 | 1.1 | 1.3 | 1.5 |
| AA470294_DNA_segment,_Chr_2,_ERATO_Doi_435,_expressed | 1.3 | 1.2 | 1.3 | 1.2 |
| AA250545_ESTs,_Weakly_simi- lar_to_SFRB_SPLICING_FACTOR_ARGININE/SERINE- RICH_11_[H.sapiens] | 1.40699* | 1.3 | 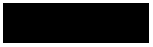 | 1.5 |
| AA869166_defensin_related_cryptdin,_related_sequence_10 | 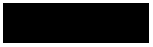 | 1.1 | 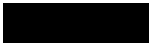 | 1.2 |
| AI608071_RIKEN_cDNA_2900017D14_gene | 1.3 | 1.2 | 1.5 | 1.5 |
| AA155097_ESTs | 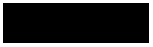 | 1.2 | 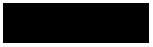 | 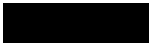 |
| AA518686_ESTs | 1.4 | 1.2 | 1.5 | 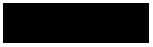 |
| AA616077_ESTs | 1.3 | 1.0 | 1.3 | 1.3 |
| AA204045_Public_domain_EST | 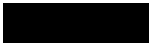 | 1.3 | 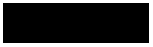 | 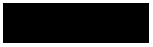 |
| AA212102_ESTs | 1.3 | 1.1 | 1.1 | 1.3 |
| AA058211_ESTs | 1.1 | 1.3 | 1.2 | 1.2 |
| AA870247_mitogene_regulated_protein,_proliferin_3 | 1.1 | 1.0 | −1.8 | 1.4 |
| AA562544_RIKEN_cDBA_1110035L05_gene | 1.4 | 1.3 | 1.5 | 1.4 |
| AA210559_ESTs,_Weakly_similar_to_T34029_hypo- | 1.2 | 1.3 | −1.0 | 1.2 |
| thetical_protein_C32F101_-_Caenorhabiditis_elegans_(D.elegans) | | | | |
| AA684073_ESTs | 1.1 | 1.2 | 1.2 | 1.3 |
| AA497627_intersectin_(SH3_domain_protein_1A) | −1.5 | −1.2 | −2.1 | −1.2 |
| AA717080_ESTs | 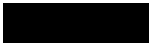 | 1.1 | 1.2 | 1.2 |
| AA244813_calbindin_2 | 1.2 | 1.3 | 1.5 | 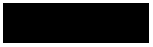 |
| AA265096_ESTs | 1.4 | 1.3 | 1.5 | 1.5 |
| AI464603_RIKEN_cDNA_0710001E13_gene | 1.4 | 1.2 | 1.4 | 1.4 |
| AA254875_ESTs | 1.3 | 1.3 | 1.2 | 1.3 |
| AA646049_interleukin_13 | 1.3 | 1.2 | 1.4 | 1.2 |
| AA290276_ESTs | 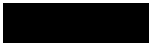 | 1.2 | 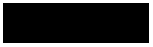 | 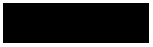 |
| AA211954_RIKEN_cDNA_4930511A05_gene | 1.4 | 1.2 | 1.1 | 1.1 |
| AI530661_RIKEN_cDNA_2810426N06_gene | 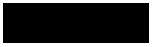 | 1.3 | 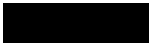 | 1.5 |
| AA571416_RIKEN_cDNA_1190004M21_gene | 1.3 | 1.3 | 1.4 | 1.3 |
| AI627071_RIKEN_cDNA_2310058A11_gene | 1.2 | 1.3 | 1.5 | 1.4 |
| AI597531_gap_junction_membrane_channel_protein_beta_2 | 1.5 | −1.2 | 1.1 | 1.0 |
| AA591707_DNA_polymerase_epsilon,_subunit_2 | 1.1 | 1.1 | −1.1 | 1.1 |
| AA473562_ESTs,_Moderately_similar_to_alias_DLC1_[H.sapiens] | 1.5 | 1.4 | 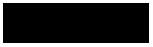 | 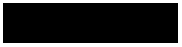 |
| AA220774_ESTs | 1.3 | 1.3 | 1.5 | 1.5 |
| AI117905_RIKEN_cDNA_6530406M24_gene | −1.3 | −1.0 | −1.5 | −1.3 |
| AA395924_ESTs | 1.5 | 1.3 | 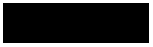 | 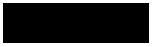 |
| AA869181_defensin_related_cryptdin_6 | 1.1 | −1.2 | 1.4 | 1.3 |
| AI604192_RIKEN_cDNA_4632433K11_gene | 1.2 | 1.1 | 1.4 | 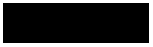 |
| AI550569_ESTs | 1.4 | 1.3 | 1.5 | 1.3 |
| AA473386_ESTs | 1.4 | 1.3 | 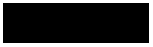 | 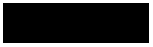 |
| AA591510_homeo_box_B1 | 1.2 | 1.1 | 1.1 | 1.3 |
| AI587821_solute_carrier_family_22_(organic_cation_trans- porter),_member_2 | 1.4 | 1.2 | 1.4 | 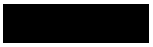 |
| AI553220_ESTs | 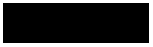 | 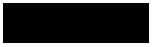 | 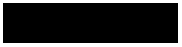 | 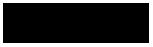 |
| [symbol] | 1.3 | 1.1 | 1.3 | 1.3 |
| AA067264_kinectin_1 | 1.3 | 1.0 | −1.1 | 1.2 |
| AA266331_ESTs | 1.2 | 1.0 | 1.3 | 1.3 |
| AA596525_chromodomain_helicase_DNA_binding_protein_1 | 1.2 | 1.3 | 1.0 | 1.3 |
| AA209628_ESTs,_Highly_similar_to_T08741_hypo- | 1.3 | 1.1 | 1.2 | 1.4 |
| thetical_protein_DKFZp586G0518.1_[H.sapiens] | | | | |
| AA259281_LIM_homeobox_protein_2 | 1.4 | 1.3 | 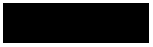 | 1.55793* |
| AI549635_ESTs | 1.4 | 1.1 | 1.4 | 1.2 |
| AA268913_flavin_containing_monooxygenase_5 | 1.3 | 1.2 | 1.2 | 1.3 |
| AA547242_undifferentiated_embryonic_cell_transcription_factor_1 | −1.5 | −1.1 | −1.8 | −1.2 |
| AA060731_nuclear_factor,_interleukin_3,_regulated | 1.5 | 1.3 | 1.3 | 1.2 |
| AI596550_leukemia/lymphoma_related_factor | 1.5 | 1.2 | 1.4 | 1.4 |
| AA822679_hematopoietic_cell_signal_transducer | 1.1 | 1.1 | 1.2 | 1.2 |
| AA517193_unc119_homolog_(C._elegans) | 1.1 | 1.0 | −1.1 | −1.1 |
| AA691755_RIKEN_cDNA_1110068E08_gene | 1.1 | 1.0 | 1.2 | 1.2 |
| AA739464_killer_cell_lectin- | 1.5 | 1.3 | 1.3 | 1.3 |
| like_receptor_subfamily_A,_member_9 | | | | |
| AA413026_Public_domain_EST | 1.5 | 1.2 | 1.3 | 1.4 |
| AA510659_ESTs | 1.4 | 1.3 | 1.4 | 1.3 |
| AA798385_ESTs | 1.3 | 1.1 | 1.4 | 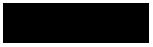 |
| AA438040_Public_domain_EST | 1.3 | 1.1 | 1.1 | 1.2 |
| AA474160_ESTs | 1.3 | 1.1 | 1.5 | 1.3 |
| AI614679_ESTs | 1.2 | 1.2 | 1.1 | 1.0 |
| AI450469_ESTs | 1.1 | 1.2 | 1.5 | 1.4 |
| AI551472_ESTs | −1.2 | −1.1 | −1.6 | −1.1 |
| AI594129_Mus_musculus,_clone_IMAGE:3494411_mRNA,_par- | 1.1 | 1.2 | 1.3 | 1.3 |
| tial_cds | | | | |
| AI390609_ESTs | 1.3 | 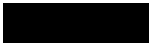 | 1.4 | 1.3 |
| AA277192_RIKEN_cDNA_2900075A18_gene | 1.1 | 1.2 | 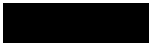 | 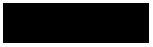 |
| AI604684_RIKEN_cDNA_4631422C13_gene | 1.4 | 1.1 | 1.2 | 1.3 |
| AI789680_ESTs | 1.1 | −1.0 | −1.0 | 1.2 |
| AA270832_ESTs | 1.2 | 1.1 | 1.5 | 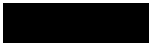 |
| AA268472_RIKEN_cDNA_2410003C20_gene | 1.1 | 1.0 | 1.3 | 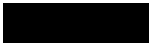 |
| AA438136_nucler_localization_signal_protein_absent_in_velo- | 1.5 | 1.3 | 1.3 | 1.3 |
| cardio-facial_patients | | | | |
| AI592746_ESTs | 1.3 | 1.1 | 1.5 | 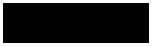 |
| AA259871_hypothetical_protein,_MNCb-4414 | 1.3 | 1.3 | 1.2 | 1.2 |
| AA710758_Mus_musculus_mRNA_b,b-carotene- 9′,10′-dioxygenase_(b-diox-II_gene) | 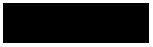 | 1.5 | 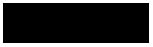 | 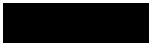 |
| AI464635_ESTs | 1.2 | 1.39713* | 1.1 | 1.2 |
| AA895221_ESTs | 1.4 | 1.1 | 1.2 | 1.5 |
| AI551957_RIKEN_cDNA_1810059J10_gene | 1.3 | 1.3 | 1.2 | 1.2 |
| AI614440_ESTs,_Moderately_similar_to_xylulokinase_[H.sapiens] | 1.4 | 1.3 | 1.1 | 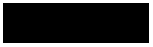 |
| AI464729_ESTs | 1.2 | 1.2 | 1.2 | 1.2 |
| AI614454_reduced_in_osteosclerosis_transporter | 1.2 | 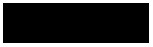 | 1.5 | 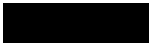 |
| AA185588_ESTs | 1.1 | 1.3 | 1.3 | 1.2 |
| AA690434_RIKEN_cDNA_2010305L05_gene | 1.3 | 1.1 | 1.4 | 1.3 |
| AA615196_ESTs | 1.175+ | 1.0364+ | 1.0 | 1.19102+ |
| AA068280_ESTs,_Weakly_similar_to_type_III_col- | −1.0 | 1.2 | −1.4 | −1.3 |
| lagen_[M.musculus] | | | | |
| AA105069_ESTs | 1.2 | 1.2 | 1.1 | 1.2 |
| AA104537_c-fos_induce_growth_factor | 1.5 | 1.5 | 1.4 | 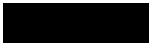 |
| AI323038_cathelin-like_protein | 1.2 | 1.2 | 1.3 | 1.3 |
| AI645164_RIKEN_cDNA_2610019N06_gene | 1.0 | 1.2 | 1.1 | 1.2 |
| AI326575_glucose-6-phosphatase,_catalytic | 1.5 | 1.1 | 1.2 | 1.5 |
| AI593604_ESTs | 1.2 | 1.1 | 1.1 | 1.3 |

STOMACH

| 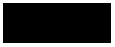 | spl | panc | liv | 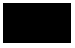 |
|---|---|---|---|---|
| Description | PL-4B_BDEnor | PL-2B_BDEnor | PL-6B_BDEnor | PL-8B_BDEnor |
| AA388685_ESTs | 1.5 | 1.2 | 1.1 | 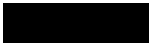 |
| AA517175_Mus_musculus_prion_inter- actor_PINT1_mRNA,_partial_cds | 1.3 | 1.5 | −1.1 | 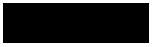 |
| AA522311_RIKEN_cDNA_0610038P07_gene | 1.2 | 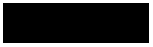 | −1.1 | 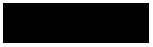 |
| AI430413_RIKEN_cDNA_1700020E22_gene | 1.2 | −1.4 | −1.2 | 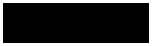 |
| AA154791_calcitonin_receptor-like | −1.1 | −1.3 | −1.1 | 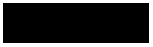 |
| AI552958_RIKEN_cDNA_1110003E08_gene | 1.0 | 1.0 | −1.1 | 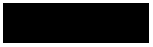 |
| AI037647_Public_domain_EST | 1.4 | 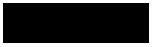 | −1.0 | 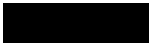 |
| AI466466_ESTs,_Weakly_similar_to_T27880_hypo- thetical_protein_ZK520.1_-_Caenorhabditis_elegans_[C.elegans] | −1.2 | 1.1 | −1.4 | 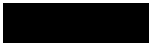 |
| AA709728_DNA_segment,_Chr_3,_ERATO_Doi_330,_expressed | 1.3 | 1.4 | −1.0 | 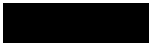 |
| AA734324_ESTs | 1.25679+ | 1.3 | −1.6 | 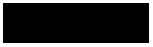 |
| AA517891_ESTs,_Weakly_similar_to_E64778_probable_mem- brane_protein_ybaL_-_Escherichia_coli_[E.coli] | 1.1 | 1.1 | −1.2 | 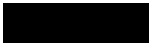 |
| AI509457_ESTs,_Highly_similar_to_KIAA0169_protein_[H.sapiens] | −1.3 | −1.0 | 1.1 | 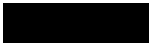 |
| AA432613_ESTs,_Moderately_simi- lar_to_1615347A_ras_p21_GTPase_activating_protein_[M.musculus] | 1.2 | 1.2 | −1.4 | 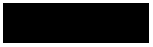 |
| AA079946_keratin_associated_protein_12-4 | −1.1 | 1.4 | −1.2 | 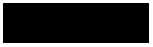 |
| AA245063_ESTs | 1.3 | 1.1 | 1.1 | 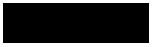 |
| AA396053_ESTs,_Moderately_similar_to_CYP2C40_[M.musculus] | 1.4 | 1.4 | 1.2 | 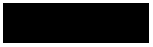 |
| AA154770_kinesin_family_member_C2 | 1.4 | 1.1 | −1.0 | 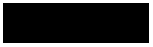 |
| AA020415_placental_specific_protein_1 | 1.2 | 1.06017+ | −1.2 | 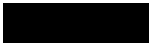 |
| AA414213_upstream_binding_protein_1 | 1.1 | −1.5 | −1.3 | −1.9 |
| AA414750_abl-interactor_1 | −1.1 | −1.2 | −1.3 | −1.9 |
| AA763355_small_EDRK-rich_factor_2 | 1.5 | 1.3 1.3 | −1.9 | |
| AA413684_RIKEN_cDNA_1300014E15_gene | 1.0 | −1.1 | 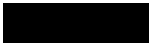 | −1.9 |
| AA414286_RIKEN_cDNA_1700016K02_gene | −1.0 | −1.24129+ | −1.7 | −1.9 |
| AA606943_glytathione_peroxidase_4 | 1.0 | 1.3 | 1.1 | −1.9 |
| AA268171_protein_phosphatase_2_(formerly_2A),_regula- | 1.0 | −1.1 | −1.0 | −1.9 |
| tory_subunit_A_(PR_65),_alpha_isoform | | | | |
| AA759679_la-associated_invariant_chain | −1.0 | −1.8 | −1.6 | −1.9 |
| W83121_legumain | 1.2 | −1.5 | 1.1 | −1.9 |
| AI594339_nuclear_factor_I/C | −1.3 | −1.2 | −1.2 | −1.9 |
| AA116994_promyelocytic_leukemia | −1.5 | −1.1 | −1.1 | −1.9 |
| AA671029_FK60_binding_protein_4_(59_kDa) | −1.1 | 1.2 | 1.0 | −1.9 |
| AA415514_microtubule- | 1.0 | −1.3 | −1.1 | −1.9 |
| associated_protein_,_RP/EB_family,_member_1 | | | | |
| AI036489_cyclin_G | −1.2 | 1.0 | 1.3 | −1.9 |
| AI048040_claudin_4 | −1.6 | 1.5 | −2.1 | −1.9 |
| AA791819_annexin_A5 | 1.3 | −1.4 | 1.1 | −1.9 |
| AA465997_RIKEN_cDNA_2010103A03_gene | 1.2 | 1.2 | 1.1 | −1.9 |
| AA980349_glutathione_peroxidase_2,_pseudogene_1 | −1.3 | −1.0 | 1.2 | −1.9 |
| AA526630_RIKEN_cDNA_1810003N24_gene | 1.2 | 1.2 | −1.1 | −1.9 |
| AI120979_cDNA_2410039E09_gene | 1.2 | 1.3 | 1.1 | −1.9 |
| AA106736_cytochrome_c_oxidase,_subunit_VIIa_3 | 1.1 | −1.0 | 1.5 | −1.9 |
| AA414844_emerin | 1.3 | 1.4 | −1.0 | −1.9 |
| AA716807_synaptophysin | −1.6 | −1.3 | −1.5 | −1.9 |
| AA154947_ESTs | −1.3 | −1.5 | −1.1 | −1.9 |
| W15971_FK506_binding_protein_2_(13_kDa) | −1.0 | −1.0 | −1.1 | −1.9 |
| AA051479_Public_domain_EST | −1.2 | −1.6 | −1.1 | −1.9 |
| AA008426_adaptor-related_protein_complex_AP- | −1.5 | −1.3 | −1.3 | −1.9 |
| 3,_sigma_2_subunit | | | | |
| W34862_glutathione_S-transferase,_mu_6 | 1.2 | 1.1 | 1.5 | −1.9 |
| AI322387_insulin-like_growth_factor_2 | −1.2 | −1.8 | −1.4 | −1.9 |
| AA727960_keratin_complex_1,_acidic,_gene_18 | 1 .0 | 1.5 | −1.2 | −1.9 |
| AI892334_ribosomal_protein_L5 | 1.3 | 1.4 | 1.1 | −1.9 |
| AA529377_heat_shock_protein,_86_kDa_1 | 1.4 | 1.0 | −1.3 | −1.9 |
| AA871749_ESTs,_Weakly_simi- | −1.5 | −1.2 | −1.4 | −1.9 |
| lar_to_I49759_Hrs_-_[M.musculus] | | | | |
| AI645415_mini_chromosome_maintenance_deficient_(S._cererisiae) | −1.3 | −1.5 | −1.3 | −1.9 |
| AA415905_nucleolar_and_coiled-body_phosphoprotein_1 | 1.1 | 1.4 | 1.3 | −1.9 |
| AI428588_RIKEN_cDNA_3110039B05_gene | −1.3 | −1.5 | −1.2 | −1.9 |
| AA067069_histocompatibility_2,_T_region_locus_9 | 1.2 | −1.2 | 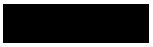 | −1.9 |
| AA674445_lectin,_galactose_binding,_soluble_9 | 1.0 | −1.0 | 1.5 | −1.9 |
| AA003005_CD151_antigen | −1.4 | −1.5 | −1.3 | −1.9 |
| AI181682_RIKEN_cDNA_3021401A05_gene | −1.2 | −1.3 | −1.3 | −1.9 |
| AI892472_RIKEN_cDNA_0610011D08_gene | −1.1 | 1.1 | 1.2 | −1.9 |
| AI385457_retinol_binding_protein_2,_cellular | −2.0 | −2.1 | −1.6 | −1.9 |
| AI049439_torsin_family_3,_member_A | −1.1 | −1.2 | −1.3 | −1.9 |
| [symbol] | −1.0 | 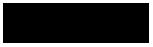 | 1.1 | −1.9 |
| AA549260_Public_domain_EST | 1.4 | 1.2 | −1.0 | −1.9 |
| AA097896_0-6-methylguanine-DNA_methyltransferase | −1.2 1.2 | −1.6 | −1.9 | |
| AA824095_RIKEN_cDNA_2610205H19_gene | −1.0 | −1.2 | 1.3 | −1.9 |
| AA619950_cyclin_I | −1.1 | −1.2 | 1.1 | −1.9 |
| AA250087_proteosome_(prosome,_macropain)_sub- | 1.1 | −1.5 | 1.1 | −1.9 |
| unit,_beta_type_8_(large_multi-functionalₚᵣₒₜₑₐₛₑ_7) | | | | |
| AA030661_kinesin_light_chain_2 | −1.3 | −1.4 | −1.4 | −1.9 |
| AI536281_toricrin | 1.2 | −1.1 | −1.3 | −1.9 |
| W10007_mast_cell_protease_6 | −1.2 | −1.4 | −13 | −1.9 |
| AA469667_tumor_rejection_antigen_gp96 | 1.3 | 1.4 | −1.5 | −1.9 |
| AA684319_protein_tyrosine_phosphatase_4a1 | 1.2 | 1.1 | 1.2 | −2.0 |
| AI553118_ESTs | −1.8 | −1.2 | −1.4 | −2.0 |
| AA500739_potassium_channel,_subfamily_K,_member_2 | −1.1 | −1.0 | −1.1 | −2.0 |
| AA600581_ribosomal_protein_L44 | 1.3 | 1.4 | 1.3 | −2.0 |
| AA684403_ESTs | −1.4 | 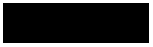 | −2.3 | −2.0 |
| AA690387_inactive_X_specific_transcripts | 1.3 | −1.04285+ | −2.3 | −2.0 |
| AA261440_lupus_nephiritis-associated_peptide_1 | 1.2 | 1.0 | 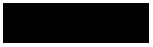 | −2.0 |
| W08321_inhibitor_of_DNA_bindind_1 | 1.1 | −1.1 | 1.1 | −2.0 |
| AA166386_transducer_of_ErbB-2.1 | −1.0 | −1.2 | 1.3 | −2.0 |
| AA437783_aplysia_ras-related_homolog_B_(RhoB) | −1.2 | −1.6 | −1.2 | −2.0 |
| AA067003_glutathione_S-transferase,_mu,_1 | 1.1 | 1.2 | 1.0 | −2.0 |
| AA066634_lactate_dehydrogenase_2,_B_chain | 1.2 | 1.2 1.33999* | −2.0 | |
| AA606826_actin,_gamma,_cytoplasmic | 1.1 | −1.2 | −1.1 | −2.0 |
| AI121405_H2.0-like_homeo_box_gene | −1.7 | −1.6 | −1.2 | −2.0 |
| AA437457_nethionine_aminopeptidase | 1.3 | −1.0 | 1.2 | −2.0 |
| W17786_spinocerebellar_ataxia_10_homlog_(human) | 1.0 | −1.1 | −1.1 | −2.0 |
| AA619894_diazepam_binding_inhibitor | 1.2 | −1.1 | −1.1 | −2.0 |
| AA185212_ESTs | −1.2 | −1.3 | −1.1 | −2.0 |
| AI893237_RIKEN_cDNA_0610009D10_gene | 1.2 | 1.2 | 1.4 | −2.0 |
| AA674592_ESTs | −1.7 | −1.3 | −1.4 | −2.0 |
| AA420091_ESTs | −1.6 | −1.7 | −1.4 | −2.0 |
| AA874467_RIKEN_cDNA_1110035G07_gene | −1.4 | −1.4 | −1.5 | −2.0 |
| AA982254_RIKEN_cDNA_2210010C04_gene | 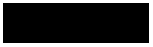 | 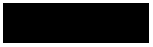 | −1.3 | −2.0 |
| AI552745_Mus_musculus,_clone_MGC:6888,_mRNA_com- | 1.1 | 1.1 | 1.1 | −2.0 |
| plete_cds | | | | |
| AA734030_opioid_receptor,_sigma_1 | −1.6 | −1.6 | −1.1 | −2.0 |
| AA920158_max_max_binding_protein | −1.6 | −1.2 | 1.06589* | −2.0 |
| AA174675_ESTs | −1.8 | −1.5 | −1.4 | −2.0 |
| AA589092_ESTs | −1.8 | −1.4 | −1.2 | −2.0 |
| W13098_natriuretic_peptide_receptor_1 | −1.8 | −1.6 | −1.6 | −2.0 |
| AA210481_clusterin | 1.1 | 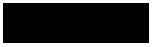 | 1.2 | −2.0 |
| AA062452_ESTs,_Moderately_similar_to_C211_HUMAN_PUTA- | 1.1 | 1.1 | 1.3 | −2.0 |
| TIVE_SURFACE_GLYCOPROTEIN_C21ORF1_PRE- | | | | |
| CURSOR_[H.sapiens] | | | | |
| AI390138_RIKEN_cDNA_3930401B19_gene | −1.4 | −10.7 | −1.1 | −2.0 |
| AA437424_Public_domain_EST | 1.0 | 1.1693+ | 1.4 | −2.0 |
| W36498_amelogenin | 1.2 | 1.2 | 1.1 | −2.0 |
| AI390970_RIKEN_cDNA_2310020F24_gene | −1.6 | −1.5 | −1.5 | −2.0 |
| AA437633_RIKEN_cDNA_2210401F17_gene | −1.2 | −1.2 | 1.4 | −2.0 |
| AA711419_POU_domain,_class_2,_transcription_factor_1 | −1.3 | 1.4 | 1.1 | −2.0 |
| AA138836_myxovirus_(influenza_virus)_resistance_1 | 1.0334+ | −1.48994+ | −1.39873+ | −2.0 |
| AA422762_ESTs | −1.4 | −1.3 | −1.2 | −2.0 |
| AA822609_ribosomal_protein_L27a | 1.5 | 1.1 | 1.3 | −2.0 |
| AA606665_Finkel-Biskis-Reilly_murine_sarcoma_virus_(FBR- | 1.2 | 1.4 | 1.1 | −2.0 |
| MuSV)_ubiquitously_expressed_(fox_derived) | | | | |
| AA465951_RIKEN_cDNA_9130022E05_gene | −1.6 | 1.6 | −1.2 | −2.0 |
| AA624488_prothymosin_alpha | 1.3 | 1.0 | 1.2 | −2.0 |
| AA771675_runt_related_transcription_factor_2 | −1.4 | −1.4 | −1.2 | −2.0 |
| W18330_tropomyosin_2,_beta | 1.2 | −1.3 | 1.0 | −2.0 |
| AI606650_calmodulin | 1.1 | 1.4 | −1.1 | −2.0 |
| AI894032_trefoil_factor_2_(spasmolytic_protein_1) | −1.1 | 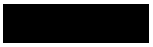 | −1.2 | −2.0 |
| AA106338_Public_domain_EST | 1.1 | 1.1 | 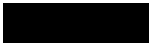 | −2.0 |
| AI047049_ESTs | −1.4 | 1.0 | −1.2 | −2.0. |
| AA414427_RIKEN_cDNA_9430015P09_gene | 1.3 | 1.2 | 1.4 | −2.0 |
| AA175784_ESTs | −1.3 | 1.02041+ | −1.1 | −2.0 |
| AA414116_signal_recognition_particle_9_kDa | 1.2 | 1.4 | 1.1 | −2.0 |
| AA008228_peroxisomal_farnesylated_protein | −1.4 | −1.9 | −1.3 | −2.1 |
| AA444231_ribosomal_protein_S5 | 1.3 | 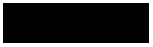 | 1.2 | −2.1 |
| AA199056_Public_domain_EST | −1.7 | −1.6 | −13 | −2.1 |
| AA619820_EST_AA536654 | 1.2 | −1.2 | 1.1 | −2.1 |
| AA471862_myosin_VI | 1.1 | −1.2 | 1.0 | −2.1 |
| AI536352_G_protein- | −1.3 | −1.7 | −1.4 | −2.1 |
| coupled_receptor,_family_C,_group_5,_member_B | | | | |
| AI604685_ubiquitin-like_(sentrin)_activating_enzyme_E1A | −1.5 | −1.4 | −1.5 | −2.1 |
| W14484_Public_domain_EST | −1.2 | −1.3 | −1.2 | −2.1 |
| AA065602_ribosomal_protein_L8 | 1.0 | 1.4 | 1.3 | −2.1 |
| AA596514_interferon_gamma_induced_GTPase | 1.2 | −1.1 | 1.5 | −2.1 |
| AI594147_beta-2_microglobulin | 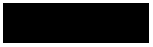 | 1.1 | 1.2 | −2.1 |
| AI326556_FMs-like_tyrosine_kinase_1 | 1.1 | −1.3 | 1.5 | −2.1 |
| AI536309_RIKEN_cDNA_2210410L06_gene | −1.8 | −1.8 | −1.4 | −2.1 |
| AA067797_heat_shock_protein_cognate_70 | 1.0 | −1.1 | 1.0 | −2.1 |
| W57231_protein_kinase_C_and_casein_kinase_sub- | −1.4 | −1.2 | −1.3 | −2.1 |
| strate_in_neurons_2 | | | | |
| AI892534_ribosomal_protein_S24 | 1.3 | 1.1 | 1.3 | −2.1 |
| AA798766_selectin,_lymphocyte | −1.7 | −1.5 | −1.2 | −2.1 |
| AI182180_three_prime_repair_exonuclease_1 | −1.4 | −1.1 | 1.1 | −2.1 |
| AA107101_prostate_stem_cell_antigen | −1.1 | −1.4 | −1.3 | −2.1 |
| AA437941_glutathione_S-transferase,_pi_2 | 1.1 1.5 | 1.2 | −2.1 | |
| AA276406_microsomal_triglyceride_transfer_protein | −1.7 | −1.4 | −1.4 | −2.1 |
| AA467239_RIKEN_cDNA_1700008C22_gene | −1.4 | −1.6 | −1.5 | −2.1 |
| AA065942_ATP_synthase,_H+_transporting_mitochon- | −1.2 | −1.0 | −1.4 | −2.1 |
| drial_F0_complex,_subunit_c_(subunit_9),_isoform_1 | | | | |
| W12636_proteaseome_(prosome,_macropain)_28_subunit,_3 | 1.1 | −1.3 | −1.3 | −2.1 |
| AA212150_eukaryotic_translation_elongation_factor_1_alpha_1 | 1.1 | 1.0 | 1.1 | −2.1 |
| AI892747_glytathione_S-transferase,_alpha_1_(Ya) | −1.1 | −1.3 | 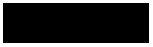 | −2.1 |
| AA667301_troponin_I,_cardiac | 1.4 | −1.6 | −1.1 | −2.1 |
| AA072834_RIKEN_cDNA_9030418MN05_gene | 1.1 | 1.2 | −1.1 | −2.1 |
| AA422639_ESTs | −1.6 | −1.7 | −1.4 | −2.1 |
| AI549639_RIKEN_cDNA_2610301D06_gene | −1.1 | 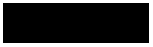 | 1.2 | −2.1 |
| AA675111_RIKEN_cDNA_1810007A24_gene | 1.4 | 1.3 | −1.0 | −2.1 |
| AI323199_tumor_necrosis_factor_receptor_superfamily,_member_4 | −1.3 | 1.2 | 1.1 | −2.1 |
| AA727065_ESTs | 1.1 | −1.2 | 1.2 | −2.1 |
| AI536342_Public_domain_EST | −1.1 | −1.1 | −1.0 | −2.1 |
| AA066534_esterase_10 | 1.1 | 1.0 | 1.2 | −2.1 |
| AA674987_RIKEN_cDNA_1110038G14_gene | −1.1 | 1.3 | −1.1 | −2.1 |
| AA210505_Public_domain_EST | 1.4 | −1.5 | 1.2 | −2.1 |
| AA123007_2′-5′_oligoadenylate_synthetase-like | 1.1 | −1.4 | −1.3 | −2.2 |
| AA607044_RIKEN_cDNA_2700099C19_gene | −1.4 | −1.3 | −1.5 | −2.2 |
| AA047991_RIKEN_cDNA_2700049I22_gene | −1.1 | 1.3 | −1.1 | −2.2 |
| W11185_ribosomal_protein_S15 | −1.1 | 1.1 | −1.2 | −2.2 |
| AI789568_potassium_voltage-gated_channel,_sub- | 1.21639+ | 1.3 | −1.42732+ | −2.2 |
| family_Q,_member_1 | | | | |
| AA437994_baculoviral_IAP_repeat-containing_4 | −1.6 | −1.5 | −1.5 | −2.2 |
| AA616180_RIKEN_cDNA_2010004B12_gene | −1.2 | 1.2 | 1.2 | −2.2 |
| W16358_mucin_1,_transmembrane | 1.5 | 1.4 | −1.2 | −2.2 |
| AA546625_ESTs,_Weakly_simi- | −1.9 | −1.6 | −1.4 | −2.2 |
| lar_to_SP62_MOUSE_SPLICEOSOME_ASSOCIATED_PRO- | | | | |
| TEIN_62_[M.musculus] | | | | |
| AA066685_villin_2 | −1.0 | −1.3 | −1.3 | −2.2 |
| AA674784_Mus_musculus_mosg_protein_(Mosg)_gene,_com- | −1.6 | −1.9 | −1.9 | −2.2 |
| plete_cds | | | | |
| AA450922_heat_shock_70kD_protein_5_(glucose- regulated_protein,_78kD) | 1.1 | 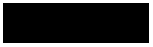 | −2.4 | −2.2 |
| AA060105_trefoil_factor_1 | −1.5 | −1.4 | 1.3 | −2.2 |
| W29311_peptidylpropyl_isomerase_C-associated_protein | 1.1 | −1.0 | 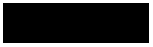 | −2.2 |
| W53728_carbonyl_reductase_1 | −1.1 | 1.2 | 1.1 | −2.2 |
| AA066404_topoisomerase_(DNA)_II_beta | 1.1 | −1.2 | 1.2 | −2.2 |
| AA879946_RIKEN_cDNA_1110015M06_gene | −1.8 | −1.2 | −1.3 | −2.2 |
| AA667785_potassium_voltage-gated_channel,_Isk- | −1.8 | −1.9 | −1.3 | −2.2 |
| related_subfamily,_member_1 | | | | |
| AA437577_ESTs | −1.9 | −1.8 | −1.5 | −2.2 |
| AA260966_ATP_synthase,_H+_transporting,_mitochon- drial_F0_complex,_subunit_g | 1.1 | −1.0 | 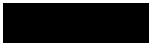 | −2.2 |
| AI551662_fos-like_antigen_2 | −2.1 | −2.0 | −1.4 | −2.2 |
| AA066282_small_inducible_cytokien_subfamily_B_(Cys- | −1.5 | −1.7 | −1.4 | −2.3 |
| X-Cys),_member_14 | | | | |
| AA426902_ESTs | −1.8 | −1.3 | −1.4 | −2.3 |
| AA106618_RIKEN_cDNA_0610007P06_gene | −1.0 | 1.1 | 1.4 | −2.3 |
| W62007_neuron_specific_gene_family_member_2 | −1.5 | −1.5 | −1.9 | −2.3 |
| AA051655_somatostalin | 1.0 | 1.0 | −1.2 | −2.3 |
| AI020170_sema_domain,_immunoglobulin_domain_(Ig),_trans- | −1.4 | −1.5 | −1.6 | −2.3 |
| membrane_domain_(TM)_and_short_cyto- | | | | |
| plasmic_domain,_(semaphorin)_4D | | | | |
| AA770808_pericentriolar_material_1 | −1.9 | −2.1 | −1.6 | −2.3 |
| AA062293_cytochrome_c_oxidase,_subunit_Va | 1.1 | 1.0 | 1.3 | −2.3 |
| W36524_schafen_2 | −1.4 | −1.3 | −1.3 | −2.3 |
| AA106154_RIKEN_cDNA_1300003O09_gene | −1.2 | −1.2 | 1.5 | −2.3 |
| AI606037_Kruppel-like_factor_2_(lung) | −1.3 | −1.4 | −1.4 | −2.3 |
| AA049653_RAD51_like_3_(S._cerevisiae) | −1.7 | −1.5 | −1.5 | −2.3 |
| AA170469_interferon- | −1.1 | −1.2 | −1.1 | −2.3 |
| induced_protein_with_tetratricopeptide_repeats_1 | | | | |
| W12985_annexin_A10 | 1.5 | −1.7 | −1.1 | −2.3 |
| AA413907_ATP_synthase,_H+transporting,_mitochon- drial_F1F0_complex,_subunit_e | 1.4 | 1.1 | 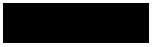 | −2.3 |
| AA14095_gene_rich_cluster,_C9_gene | −1.7 | −1.4 | −1.5 | −2.3 |
| AI876997_carboxyl_ester_lipase | 1.5 | 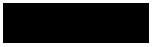 | −1.4 | −2.3 |
| AA444672_RIKEN_cDNA_2510001A17_gene | −1.1 | −1.0 | 1.1 | −2.3 |
| AA049003_heterogeneous_nuclear_ribonucleoprotein_D | 1.0 | −1.6 | −1.6 | −2.3 |
| AI325332_transglutaminase_3,_E_polypeptide | −2.0 | −2.0 | −1.4 | −2.3 |
| AA105866_glutathione_S-transferase,_alpha_4 | 1.1 | 1.0 | 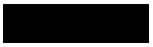 | −2.4 |
| AA822098_aplipoprotein_A-I | −1.9 | −1.5 | 1.1 | −2.4 |
| AA619848_RIKEN_cDNA_4430402G14_gene | 1.5 | −1.3 | −1.1 | −2.4 |
| AA616014_ESTs | −1.8 | −1.6 | −1.4 | −2.4 |
| AI593751_ESTs,_Highly_similar_to_AF091457_1_zinc_fin- | 1.5 | −1.2 | −1.0 | −2.4 |
| ger_protein_RIN_ZF_[R.norvegicus] | | | | |
| AA718476_RIKEN_cDNA_0610025G13_gene | 1.2 | 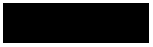 | 1.3 | −2.5 |
| AA770709_aldehyde_dehydrogenase_family_3,_subfamily_A1 | −1.1 | 1.1 | −1.2 | −2.5 |
| AA437620_RIKEN_cDNA_1810030E05_gene | −1.4 | −1.6 | −1.4 | −2.5 |
| AA727627_ankyrin_1,_erythroid | −1.8 | −2.1 | −1.6 | −2.5 |
| AI451862_RIKEN_cDNA_0910001A18_gene | 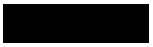 | 1.3 | −1.1 | −2.5 |
| W15606_dynein,_axon,_heavy_chain_11 | 1.3 | −1.3 | −1.1 | −2.5 |
| AA437871_eukaryotic_translation_initiation_factor_4,_gamma_2 | 1.1 | 1.0 | 1.4 | −2.5 |
| AI893971_zinc_finger_protein_57 | −1.8 | −1.8 | −1.4 | −2.5 |
| AI594148_ribosomal_protein_S19 | 1.2 | 1.1 | 1.2 | −2.5 |
| AA048952_solute_carrier_family_4_(anion_exchanger),_member_2 | −1.0 | 1.1 | 1.1 | −2.5 |
| AI117832_thymidylate_kinase_family_LPS-inducible_member | 1.2 | −1.10107+ | −1.1 | −2.5 |
| AA444254_ornithine_decarboxylase,_structural | 1.1 | −1.3 | −1.4 | −2.5 |
| AI122517_interferon_regulatory_factor_7 | 1.3 −1.3 | −1.1 | −2.6 | |
| AI322733_2′-5′_oligodenylate_synthetase_1A | 1.1 | 1.2 | −1.1 | −2.6 |
| AA062440_quaking | 1.3 | 1.3 | 1.4 | −2.6 |
| AA245976_dihydrolipoamide_dehydrogenase | 1.0 | 1.5 | 1.1 | −2.6 |
| AI551576_ESTs | −1.1 | 1.0 | 1.2 | −2.6 |
| AA717025_Mus_musculus_10_day_old_male_pan- crease_cDNA_full-length_enriched_library,_clone:1810009A17,_full_in- sert_sequence | 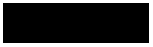 | −1.1 | −1.2 | −2.7 |
| AI464778_ESTs | −1.9 | −1.6 | −1.6 | −2.7 |
| AA674270_major_urinary_protein_1 | 1.1 | −1.1 | −5.01013* | −2.7 |
| AA175695_RIKEn_cDNA_5730455P16_gene | −1.3 | −1.1 | 1.2 | −2.7 |
| AA675084_elastase_2 | 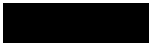 | 1.4 | −1.2 | −2.7 |
| AA555905_ribosomal_protein_L28 | −1.3 | 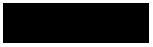 | −1.0793* | −2.7 |
| AI120876_CD24a_antigen | 1.3 | 1.2 | −1.5 | −2.8 |
| AA414790_leukotriene_A4_hydrolase | −2.1 | −2.2 | −1.6 | −2.8 |
| AA821884_amylase_2,_pancreatic | 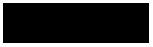 | 1.1 | −1.2 | −2.8 |
| AA185052_RIKEN_cDNA_2510004L01_gene | 1.0 | 1.0 | −1.1 | −2.8 |
| AA733740_ESTs | −2.0 | −2.2 | −1.6 | −2.9 |
| W15809_hemoglobin,_beta_adult_major_chain | 1.5 | −1.2 | 1.2 | −2.9 |
| AA122791_histocompatibility_2_Q_region_locus_7 | 1.3 | −1.4 | 1.3 | −3.0 |
| AA106071_hemoglobin,_beta_adult_major_chain | 1.3 | 1.4 | 1.2 | −3.0 |
| W14953_kinesin-associated_protein_3 | −1.0 | −1.0 | 1.4 | −3.2 |
| AA727519_interferon-induced_protein_with_tetra- | 1.3 | −1.1 | 1.1 | −3.3 |
| tricopeptide_repeats_3 | | | | |
| AI324398_glycosylphosphatidylinositol_specific_phospholipase_D1 | 1.1 | −1.3 | 1.2 | −3.4 |
| AI326722_lymphocyte_antigen_6_complex,_locus_E | −1.1 | −1.2 | −1.2 | −3.5 |
| AA396056_ubiquitin_specific_protease_18 | 1.1 | 1.0 | 1.2 | −3.6 |
| AA237793_rat_regenerating_islet-derived,_mouse_homolog_1 | 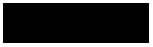 | 1.4 | −1.3 | −3.9 |
| AA066763_hemoglobin,_beta_adult_major_chain | 1.3 | −1.2 | 1.4 | −4.0 |
| AA796818_interferon-stimulated_protein_(15_kDa) | 1.0 | −1.6 | −1.2 | −5.2 |
| AA790398_ribonuclease_1,_pancreatic | 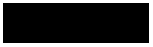 | 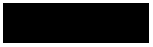 | −1.2 | −5.8 |
| AA175618_interferon-induced_protein_with_tetra- | −1.1 | −1.65103+ | −1.2 | −7.4 |
| tricopeptide_repeats_1 | | | | |
| AA822105_major_urinary_protein_1 | −1.1 | −2.4 | −28.7649* | −7.8 |

STOMACH

| 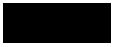 | int | col | brain | lung |
|---|---|---|---|---|
| Description | PL-3B_BDEnor | PL-5B_BDEnor | PL-1B_BDEnor | PL-7B_BDEnor |
| AA388685_ESTs | 1.4 | −1.12144+˜ | 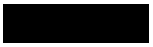 | 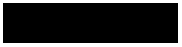 |
| AA517175_Mus_musculus_prion_inter- actor_PINT1_mRNA,_partial_cds | 1.4 | −1.3 | 1.2 | 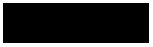 |
| AA522311_RIKEN_cDNA_0610038P07_gene | 1.2 | −1.08749+˜ | 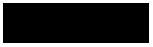 | 1.3 |
| AI430413_RIKEN_cDNA_1700020E22_gene | 1.3 | 1.3 | −1.1 | −1.0 |
| AA154791_calcitonin_receptor-like | 1.2 −1.12718+˜ | −1.12138+ | −1.3 | |
| AI552958_RIKEN_cDNA_1110003E08_gene | 1.4 | −1.14793˜ | 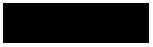 | 1.1 |
| AI037647_Public_domain_EST | 1.3 | −1.1 | 1.1 | 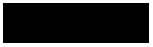 |
| AI466466_ESTs,_Weakly_similar_to_T27880_hypo- thetical_protein_ZK520.1_-_Caenorhabditis_elegans_[C.elegans] | 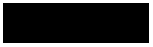 | 1.52355+˜ | 1.3 | 1.2 |
| AA709728_DNA_segment,_Chr_3,_ERATO_Doi_330,_expressed | 1.5 | 1.07517* | 1.0 | 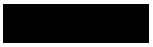 |
| AA734324_ESTs | 1.2 | −1.06995+˜ | −1.1 | 1.5 |
| AA517891_ESTs,_Weakly_similar_to_E64778_probable_mem- | 1.2 | −2.00749˜ | 1.1 | 1.4 |
| brane_protein_ybaL_-_Escherichia_coli_[E.coli] | | | | |
| AI509457_ESTs,_Highly_similar_to_KIAA0169_protein_[H.sapiens] | 1.2 | −1.26078+˜ | −1.1 | 1.3 |
| AA432613_ESTs,_Moderately_simi- lar_to_1615347A_ras_p21_GTPase_activating_protein_[M.musculus] | 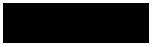 | 1.4225˜ | 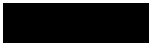 | 1.3 |
| AA079946_keratin_associated_protein_12-4 | 1.4 | −1.07+˜ | 1.0 | 1.3 |
| AA245063_ESTs | 1.4 | −1.0806+˜ | 1.3 | 1.04052+ |
| AA396053_ESTs,_Moderately_similar_to_CYP2C40_[M.musculus] | 1.2 | −1.0 | 1.2 | 1.4 |
| AA154770_kinesin_family_member_C2 | 1.2 | −1.07647+˜ | 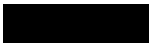 | 1.2 |
| AA020415_placental_specific_protein_1 | 1.2 | −1.16409+˜ | 1.47888+ | 1.09682+ |
| AA414213_upstream_binding_protein_1 | −1.3 | −1.0 | −1.3 | −1.3 |
| AA414750_abl-interactor_1 | −1.4 | −1.1 | 1.2 | −1.1 |
| AA763355_small_EDRK-rich_factor_2 | 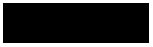 | 1.2 | −1.2 | −1.2 |
| AA413684_RIKEN_cDNA_1300014E15_gene | −1.4 | 1.25078+˜ | −1.3 | −1.1 |
| AA414286_RIKEN_cDNA_1700016K02_gene | −1.1 | 1.0257+˜ | −1.16076+ | −1.62152+ |
| AA606943_glytathione_peroxidase_4 | 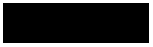 | 1.4 | −1.0 | −1.7 |
| AA268171_protein_phosphatase_2_(formerly_2A),_regula- | 1.3 | −1.1 | 1.1 | 1.1 |
| tory_subunit_A_(PR_65),_alpha_isoform | | | | |
| AA759679_la-associated_invariant_chain | 1.5 | −3.7 | −2.8 | −1.0 |
| W83121_legumain | −1.0 | −1.7 | −1.0 | −1.3 |
| AI594339_nuclear_factor_I/C | 1.0 | 1.1 | −1.2 | −1.0 |
| AA116994_promyelocytic_leukemia | −1.1 | −1.6 | −1.3 | −1.2 |
| AA671029_FK60_binding_protein_4_(59_kDa) | −1.4 | −1.4 | −1.7 | −1.0 |
| AA415514_microtubule- | −1.2 | −1.4 | 1.3 | 1.0 |
| associated_protein_,_RP/EB_family,_member_1 | | | | |
| AI036489_cyclin_G | −1.8 | −2.26973˜ | 1.2 | −1.4 |
| AI048040_claudin_4 | 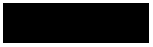 | −2.7 | 1.5 | −1.1 |
| AA791819_annexin_A5 | −1.0 | −1.0 | 1.0 | 1.2 |
| AA465997_RIKEN_cDNA_2010103A03_gene | −1.2 | −1.5 | 1.1 | 1.2 |
| AA980349_glutathione_peroxidase_2,_pseudogene_1 | 1.1 | −2.4 | −2.5 | −1.5 |
| AA415520_RIKEN_cDNA_1810003N24_gene | −1.1 | −1.42227+˜ | 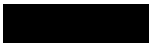 | 1.1 |
| AI120979_cDNA_2410039E09_gene | −1.6 | −1.3 | 1.0 | −1.6 |
| AA106736_cytochrome_c_oxidase,_subunit_VIIa_3 | −1.2 | −1.1 | 1.0 | −1.3 |
| AA414844_emerin | 1.1 | −1.0 | −1.1 | −1.1 |
| AA716807_synaptophysin | −1.3 | −1.3 | 1.4 | −1.7 |
| AA154947_ESTs | −1.1 | −1.0 | −1.1 | −1.1 |
| W15971_FK506_binding_protein_2_(13_kDa) | −1.2 | −1.4 | −1.2 | −1.4 |
| AA051479_Public_domain_EST | −1.6 | −1.1 | −1.1 | −1.3 |
| AA008426_adaptor-related_protein_complex_AP- | −1.1 | −1.2 | −1.0 | −1.1 |
| 3,_sigma_2_subunit | | | | |
| W34862_glutathione_S-transferase,_mu_6 | 1.3 | 1.2 | 1.2 | −1.1 |
| AI322387_insulin-like_growth_factor_2 | −1.5 | 1.4 | −1.5 | −2.6 |
| AA727960_keratin_complex_1,_acidic,_gene_18 | −1.1 | 1.0 | −1.7 | −1.0 |
| AI892334_ribosomal_protein_L5 | −1.2 | 1.1 | 1.2 | 1.1 |
| AA529377_heat_shock_protein,_86_kDa_1 | −1.9 | −1.5 | 1.4 | 1.3 |
| AA871749_ESTs,_Weakly_simi- | −1.1 | −1.4 | 1.3 | −1.4 |
| lar_to_I49759_Hrs_-_[M.musculus] | | | | |
| AI645415_mini_chromosome_maintenance_deficient_(S._cererisiae) | −1.4 | −1.2 | −1.8 | −1.4 |
| AA415905_nucleolar_and_coiled-body_phosphoprotein_1 | 1.2 | −1.0 | −1.4 | −1.0 |
| AI428588_RIKEN_cDNA_3110039B05_gene | −1.2 | 1.4 | 1.0 | −1.3 |
| AA067069_histocompatibility_2,_T_region_locus_9 | 1.4 | −1.3 | 1.1 | −1.1 |
| AA674445_lectin,_galactose_binding,_soluble_9 | −1.2 | −1.7 | −1.4 | −1.0 |
| AA003005_CD151_antigen | −1.1 | −1.3 | −1.2 | −1.2 |
| AI181682_RIKEN_cDNA_3021401A05_gene | −1.4 | −1.5 | 1.1 | −1.1 |
| AI892472_RIKEN_cDNA_0610011D08_gene | −1.6 | −1.2 | −1.5 | −1.2 |
| AI385457_retinol_binding_protein_2,_cellular | 1.5 | 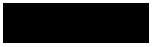 | −1.3 | −1.9 |
| AI049439_torsin_family_3,_member_A | −1.2 | −2.24409+˜ | −1.0 | 1.1 |
| [symbol] | 1.1 | −1.2 | 1.2 | 1.0 |
| AA549260_Public_domain_EST | −1.56322+ | −1.27906+˜ | 1.5 | −1.1 |
| AA097896_0-6-methylguanine-DNA_methyltransferase | 1.3 | −2.3 | 1.0 | −1.1 |
| AA824095_RIKEN_cDNA_2610205H19_gene | −1.3 | 1.2 | −1.7 | −1.9 |
| AA619950_cyclin_I | −1.1 | −1.1 | 1.5 | −1.4 |
| AA250087_proteosome_(prosome,_macropain)_sub- | 1.0 | −3.6 | −1.5 | −1.2 |
| unit,_beta_type_8_(large_multi-functionalₚᵣₒₜₑₐₛₑ_7) | | | | |
| AA030661_kinesin_light_chain_2 | −1.1 | −1.4 | 1.2 | −1.4 |
| AI536281_toricrin | 1.3 | 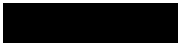 | −1.2 | 1.3 |
| W10007_mast_cell_protease_6 | −1.2 | −12. | −1.4 | −1.2 |
| AA469667_tumor_rejection_antigen_gp96 | −2.0 | −1.4 | 1.2 | −1.1 |
| AA684319_protein_tyrosine_phosphatase_4a1 | −1.6 | −1.2 | 1.3 | 1.1 |
| AI553118_ESTs | 1.1 | −1.3 | −1.1 | −1.3 |
| AA500739_potassium_channel,_subfamily_K,_member_2 | −1.7 | −1.24216˜ | 1.4 | −1.0 |
| AA600581_ribosomal_protein_L44 | −1.7 | −1.2 | 1.2 | −1.4 |
| AA684403_ESTs | 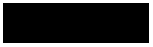 | −2.4 | 1.5 | −1.0 |
| AA690387_inactive_X_specific_transcripts | −1.3 | 1.15376+˜ | −1.33094+ | −1.2 |
| AA261440_lupus_nephiritis-associated_peptide_1 | −1.4 | −1.1 | 1.1 | −1.6 |
| W08321_inhibitor_of_DNA_bindind_1 | −1.1 | −2.7 | −1.6 | −1.1 |
| AA166386_transducer_of_ErbB-2.1 | −1.9 | −1.1 | 1.1 | −1.2 |
| AA437783_aplysia_ras-related_homolog_B_(RhoB) | −1.0 | −1.5 | −1.0 | −1.2 |
| AA067003_glutathione_S-transferase,_mu,_1 | 1.5 | 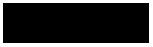 | 1.4 | 1.0 |
| AA066634_lactate_dehydrogenase_2,_B_chain | −1.3 | 1.4 | 1.3 | 1.0 |
| AA606826_actin,_gamma,_cytoplasmic | −1.0 | −1.4 | 1.4 | 1.1 |
| AI121405_H2.0-like_homeo_box_gene | −1.1 | −1.6 | −1.2 | −1.2 |
| AA437457_nethionine_aminopeptidase | −1.1 | −1.1 | −1.4 | −1.0 |
| W17786_spinocerebellar_ataxia_10_homlog_(human) | −1.4 | −1.3 | 1.2 | 1.0 |
| AA619894_diazepam_binding_inhibitor | −1.0 | −1.1 | 1.2 | −1.7 |
| AA185212_ESTs | −1.1 | −1.3 | −1.2 | −1.2 |
| AI893237_RIKEN_cDNA_0610009D10_gene | −1.0 | −1.3 | 1.4 | −1.3 |
| AA674592_ESTs | −1.1 | −1.31129˜ | 1.1 | −1.6 |
| AA420091_ESTs | −1.4 | −1.4 | −1.2 | −1.4 |
| AA874467_RIKEN_cDNA_1110035G07_gene | −1.1 | −1.3 | 1.1 | −1.3 |
| AA982254_RIKEN_cDNA_2210010C04_gene | −9.5 | −1.3 | 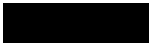 | −1.5 |
| AI552745_Mus_musculus,_clone_MGC:6888,_mRNA_com- | −2.0 | 1.1 | −1.2 | −1.5 |
| plete_cds | | | | |
| AA734030_opioid_receptor,_sigma_1 | −1.1 | −1.1 | −1.2 | −1.5 |
| AA920158_max_max_binding_protein | −1.2 | 1.3 | −1.1 | −1.1 |
| AA174675_ESTs | −1.3 | −1.2 | −1.1 | −1.3 |
| AA589092_ESTs | −1.1 | −1.3 | −1.3 | −1.4 |
| W13098_natriuretic_peptide_receptor_1 | −1.2 | −1.4 | −1.1 | −1.1 |
| AA210481_clusterin | −1.8 | −1.4 | 1.3 | −1.1 |
| AA062452_ESTs,_Moderately_similar_to_C211_HUMAN_PUTA- | −1.1 | 1.1 | 1.3 | −1.2 |
| TIVE_SURFACE_GLYCOPROTEIN_C21ORF1_PRE- | | | | |
| CURSOR_[H.sapiens] | | | | |
| AI390138_RIKEN_cDNA_3930401B19_gene | −1.2 | −2.1 | −1.3 | −1.2 |
| AA437424_Public_domain_EST | −1.2 | −1.16472+˜ | 1.31017+ | −1.3034+ |
| W36498_amelogenin | 1.1 | −1.3 | 1.1 | −1.1 |
| AI390970_RIKEN_cDNA_2310020F24_gene | −1.1 | −1.36726˜ | −1.0 | −1.3 |
| AA437633_RIKEN_cDNA_2210401F17_gene | −1.1 | 1.0 | −1.3 | −1.7 |
| AA711419_POU_domain,_class_2,_transcription_factor_1 | 1.1 | −1.08813˜ | −1.4 | 1.0 |
| AA138836_myxovirus_(influenza_virus)_resistance_1 | −1.18242+ | 1.16979+˜ | −1.06713+ | 1.1 |
| AA422762_ESTs | −1.3 | −1.2 | −1.0 | −1.1 |
| AA822609_ribosomal_protein_L27a | −1.5 | 1.0 | 1.2 | −1.1 |
| AA606665_Finkel-Biskis-Reilly_murine_sarcoma_virus_(FBR- | 1.1 | 1.00729+**˜ | −1.1 | −1.2 |
| MuSV)_ubiquitously_expressed_(fox_derived) | | | | |
| AA465951_RIKEN_cDNA_9130022E05_gene | −1.2 | −1.0 | −1.1 | −1.1 |
| AA624488_prothymosin_alpha | −1.8 | −1.2 | −1.1 | −1.2 |
| AA771675_runt_related_transcription_factor_2 | −1.4 | −1.3 | −1.1 | −1.2 |
| W18330_tropomyosin_2,_beta | 1.2 | −1.1 | −3.5 | −1.5 |
| AI606650_calmodulin | 1.5 | −1.1 | 1.3 | 1.2 |
| AI894032_trefoil_factor_2_(spasmolytic_protein_1) | −82.1 | −1.89777˜ | −1.4 | 1.0 |
| AA106338_Public_domain_EST | 1.3 | −1.1 | 1.0 | −1.1 |
| AI047049_ESTs | 1.0 | −1.29793+˜ | −1.0 | −1.3 |
| AA414427_RIKEN_cDNA_9430015P09_gene | −1.8 | −1.2 | 1.1 | −1.0 |
| AA175784_ESTs | −1.1 | −1.20227+˜ | 1.05224+ | 1.08166+ |
| AA414116_signal_recognition_particle_9_kDa | −1.3 | −1.4 | −1.3 | −1.4 |
| AA008228_peroxisomal_farnesylated_protein | 1.0 | −1.1 | −1.2 | −1.2 |
| AA444231_ribosomal_protein_S5 | 1.1 | −1.3 | 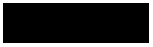 | −1.1 |
| AA199056_Public_domain_EST | −1.1 | −1.1 | −1.0 | −1.4 |
| AA619820_EST_AA536654 | 1.1 | −1.6 | −1.0 | 1.1 |
| AA471862_myosin_VI | −1.2 | 1.1 | 1.1 | 1.0 |
| AI536352_G_protein- | −2.9 | −1.3 | −1.6 | −1.1 |
| coupled_receptor,_family_C,_group_5,_member_B | | | | |
| AI604685_ubiquitin-like_(sentrin)_activating_enzyme_E1A | 1.20761* | −1.4 | 1.0 | −1.5 |
| W14484_Public_domain_EST | 1.0 | 1.2 | −1.0 | −1.0 |
| AA065602_ribosomal_protein_L8 | 1.1 | −1.0 | 1.1 | −1.3 |
| AA596514_interferon_gamma_induced_GTPase | −1.2 | −1.58973˜ | −1.5 | −1.6 |
| AI594147_beta-2_microglobulin | 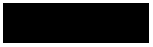 | −1.8 | −1.0 | −1.1 |
| AI326556_FMs-like_tyrosine_kinase_1 | −1.1 | 1.0 | 1.1 | −1.0 |
| AI536309_RIKEN_cDNA_2210410L06_gene | −1.9 | 1.1 | −1.5 | −2.0 |
| AA067797_heat_shock_protein_cognate_70 | −2.1 | −1.5 | −1.1 | 1.3 |
| W57231_protein_kinase_C_and_casein_kinase_sub- | 1.2 | −1.4 | −1.6 | −1.1 |
| strate_in_neurons_2 | | | | |
| AI892534_ribosomal_protein_S24 | −2.0 | −1.1 | 1.3 | −1.2 |
| AA798766_selectin,_lymphocyte | −1.0 | −1.2 | 1.0 | −1.5 |
| AI182180_three_prime_repair_exonuclease_1 | 1.0 | −1.24935+˜ | −1.4 | 1.2 |
| AA107101_prostate_stem_cell_antigen | −6.1 | 1.51614˜ | −1.8 | −2.1 |
| AA437941_glutathione_S-transferase,_pi_2 | 1.5 | 1.4 | 1.1 | −1.6 |
| AA276406_microsomal_triglyceride_transfer_protein | 1.0 | 1.2 | 1.0 | −1.6 |
| AA467239_RIKEN_cDNA_1700008C22_gene | −1.2 | −1.2 | 1.0 | −1.5 |
| AA065942_ATP_synthase,_H+_transporting_mitochon- | −1.2 | −1.5 | −1.3 | −1.4 |
| drial_F0_complex,_subunit_c_(subunit_9),_isoform_1 | | | | |
| W12636_proteaseome_(prosome,_macropain)_28_subunit,_3 | 1.1 | −1.1 | −1.0 | −1.1 |
| AA212150_eukaryotic_translation_elongation_factor_1_alpha_1 | −1.2 | −1.2 | 1.2 | 1.1 |
| AI892747_glytathione_S-transferase,_alpha_1_(Ya) | −1.7 | 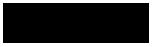 | −1.2 | −1.1 |
| AA667301_troponin_I,_cardiac | 1.3 | 1.41297˜ | −2.0 | −2.0 |
| AA072834_RIKEN_cDNA_9030418MN05_gene | 1.13744* | −3.3 | −1.1 | 1.1 |
| AA422639_ESTs | −1.3 | −1.3 | −1.2 | −1.2 |
| AI549639_RIKEN_cDNA_2610301D06_gene | −1.1 | −1.1 | 1.3 | −1.1 |
| AA675111_RIKEN_cDNA_1810007A24_gene | −14.0 | −1.5 | 1.0 | −1.4 |
| AI323199_tumor_necrosis_factor_receptor_superfamily,_member_4 | −1.2 | −1.14522+˜ | 1.1 | −1.1 |
| AA727065_ESTs | −2.5 | 1.2 | −1.2 | −1.6 |
| AI536342_Public_domain_EST | 1.2 | 1.0 | 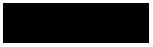 | 1.4 |
| AA066534_esterase_10 | 1.0 | 1.1 | 1.1 | −1.1 |
| AA674987_RIKEN_cDNA_1110038G14_gene | 1.2 | 1.5 | −1.3 | −2.1 |
| AA210505_Public_domain_EST | −2.7 | −1.7 | −1.1 | −1.1 |
| AA123007_2′-5′_oligoadenylate_synthetase-like | −1.1 | −2.2 | −1.1 | 1.3 |
| AA607044_RIKEN_cDNA_2700099C19_gene | −1.0 | −1.4 | −1.1 | −1.4 |
| AA047991_RIKEN_cDNA_2700049I22_gene | −1.0 | −1.0 | −1.3 | −1.4 |
| W11185_ribosomal_protein_S15 | −1.0 | −1.2 | −1.2 | −1.3 |
| AI789568_potassium_voltage-gated_channel,_sub- | 1.0 | 1.08585+˜ | −1.33325+ | 1.31433+ |
| family_Q,_member_1 | | | | |
| AA437994_baculoviral_IAP_repeat-containing_4 | −1.2 | −1.38447˜ | 1.2 | −1.1 |
| AA616180_RIKEN_cDNA_2010004B12_gene | −1.4 | −1.14572+˜ | 1.1 | −1.3 |
| W16358_mucin_1,_transmembrane | −1.3 | −1.1 | −2.0 | 1.0 |
| AA546625_ESTs,_Weakly_simi- | −1.2 | −1.4 | 1.2 | −1.2 |
| lar_to_SP62_MOUSE_SPLICEOSOME_ASSOCIATED_PRO- | | | | |
| TEIN_62_[M.musculus] | | | | |
| AA066685_villin_2 | 1.3 | 1.2 | −1.2 | −1.1 |
| AA674784_Mus_musculus_mosg_protein_(Mosg)_gene,_com- | −1.0 | −1.1 | −1.2 | −1.4 |
| plete_cds | | | | |
| AA450922_heat_shock_70kD_protein_5_(glucose- | −2.4 | −1.6 | 1.0 | −1.3 |
| regulated_protein,_78kD) | | | | |
| AA060105_trefoil_factor_1 | −43.1146* | −1.06656˜ | 1.0 | −1.2 |
| W29311_peptidylpropyl_isomerase_C-associated_protein | −1.2 | −1.4 | 1.1 | −1.0 |
| W53728_carbonyl_reductase_1 | 1.1 | 1.1 | −1.2 | −1.5 |
| AA066404_topoisomerase_(DNA)_II_beta | −2.2 | 1.0 | −1.2 | −1.5 |
| AA879946_RIKEN_cDNA_1110015M06_gene | −1.2 | −1.3 | 1.3 | −1.6 |
| AA667785_potassium_voltage-gated_channel,_Isk- | −1.2 | −1.0 | −1.3 | −1.4 |
| related_subfamily,_member_1 | | | | |
| AA437577_ESTs | −1.2 | −1.47043˜ | −1.53147+ | −1.5 |
| AA260966_ATP_synthase,_H+_transporting,_mitochon- | −1.4 | −1.2 | −1.2 | −1.7 |
| drial_F0_complex,_subunit_g | | | | |
| AI551662_fos-like_antigen_2 | −1.2 | −1.1 | −1.3 | −1.4 |
| AA066282_small_inducible_cytokien_subfamily_B_(Cys- | −1.1 | −1.0 | −1.1 | −1.4 |
| X-Cys),_member_14 | | | | |
| AA426902_ESTs | 1.0 | −1.4 | −1.1 | −1.3 |
| AA106618_RIKEN_cDNA_0610007P06_gene | −1.3 | 1.21425*˜ | −1.2 | −1.1 |
| W62007_neuron_specific_gene_family_member_2 | −1.3 | −1.0 | 1.4 | −2.0 |
| AA051655_somatostalin | 1.2 | −1.0 | −1.3 | −1.0 |
| AI020170_sema_domain,_immunoglobulin_domain_(Ig),_trans- | −1.1 | −1.2 | −1.2 | −1.6 |
| membrane_domain_(TM)_and_short_cyto- | | | | |
| plasmic_domain,_(semaphorin)_4D | | | | |
| AA770808_pericentriolar_material_1 | −1.1 | −1.2 | −1.0 | −1.5 |
| AA062293_cytochrome_c_oxidase,_subunit_Va | −1.2 | −1.0 | −1.2 | −1.1 |
| W36524_schafen_2 | −1.4 | −1.4 | −1.2 | −1.1 |
| AA106154_RIKEN_cDNA_1300003O09_gene | −1.3 | 1.2 | 1.1 | −1.4 |
| AI606037_Kruppel-like_factor_2_(lung) | −1.1 | −1.5 | −1.1 | 1.1 |
| AA049653_RAD51_like_3_(S._cerevisiae) | −1.1 | −1.5 | −1.3 | −1.3 |
| AA170469_interferon- | −1.6 | −1.20626+˜ | −1.4 | 1.0 |
| induced_protein_with_tetratricopeptide_repeats_1 | | | | |
| W12985_annexin_A10 | −8.5 | −1.03567˜ | −1.3 | −1.1 |
| AA413907_ATP_synthase,_H+transporting,_mitochon- | −1.4 | −1.0 | 1.0 | −1.7 |
| drial_F1F0_complex,_subunit_e | | | | |
| AA14095_gene_rich_cluster,_C9_gene | −1.1 | −1.3 | 1.1 | −1.5 |
| AI876997_carboxyl_ester_lipase | −8.9 | −1.9 | −1.3 | −1.4 |
| AA444672_RIKEN_cDNA_2510001A17_gene | −2.0 | −1.1 | 1.3 | −1.1 |
| AA049003_heterogeneous_nuclear_ribonucleoprotein_D | −1.1 | −1.2 | −1.2 | −1.3 |
| AI325332_transglutaminase_3,_E_polypeptide | −1.2 | −1.3 | −1.2 | −1.2 |
| AA105866_glutathione_S-transferase,_alpha_4 | −3.0 | 1.3 | 1.2 | 1.0 |
| AA822098_aplipoprotein_A-I | 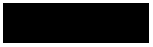 | 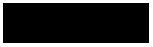 | −1.9 | −9.0 |
| AA619848_RIKEN_cDNA_4430402G14_gene | −1.1 | −1.1 | −1.1 | −1.1 |
| AA616014_ESTs | −1.2 | −1.5 | −1.3 | −1.4 |
| AI593751_ESTs,_Highly_similar_to_AF091457_1_zinc_fin- | 1.0 | −1.0 | −1.1 | −1.1 |
| ger_protein_RIN_ZF_[R.norvegicus] | | | | |
| AA718476_RIKEN_cDNA_0610025G13_gene | 1.1 | −1.1 | 1.1 | −1.4 |
| AA770709_aldehyde_dehydrogenase_family_3,_subfamily_A1 | 1.3 | −1.6108˜ | 1.5 | −1.0 |
| AA437620_RIKEN_cDNA_1810030E05_gene | −1.2 | −1.0 | −1.1 | −1.3 |
| AA727627_ankyrin_1,_erythroid | −1.3 | −1.2 | −1.4 | −1.4 |
| AI451862_RIKEN_cDNA_0910001A18_gene | −14.7 | −2.6 | 1.2 | −1.3 |
| W15606_dynein,_axon,_heavy_chain_11 | −2.1 | −1.8 | −1.1 | 1.0 |
| AA437871_eukaryotic_translation_initiation_factor_4,_gamma_2 | −1.7 | −1.0 | 1.2 | −1.0 |
| AI893971_zinc_finger_protein_57 | −1.2 | −1.2 | −1.3 | −1.3 |
| AI594148_ribosomal_protein_S19 | −1.4 | −1.0 | 1.1 | −1.4 |
| AA048952_solute_carrier_family_4_(anion_exchanger),_member_2 | −1.1 | −1.5 | −1.4 | −1.0 |
| AI117832_thymidylate_kinase_family_LPS-inducible_member | 1.3 | −1.3814+˜ | 1.4 | 1.1 |
| AA444254_ornithine_decarboxylase,_structural | −1.6 | −1.2 | −1.1 | −1.6 |
| AI122517_interferon_regulatory_factor_7 | 1.2 | −1.85968˜ | −1.2 | −1.0 |
| AI322733_2′-5′_oligodenylate_synthetase_1A | 1.1 | −2.1 | −1.1 | 1.0 |
| AA062440_quaking | −1.2 | −1.2 | −1.0 | −1.3 |
| AA245976_dihydrolipoamide_dehydrogenase | 1.2 | −1.1 | 1.4 | −1.1 |
| AI551576_ESTs | −1.8 | 1.3 | −1.1 | −1.0 |
| AA717025_Mus_musculus_10_day_old_male_pan- crease_cDNA_full-length_enriched_library,_clone:1810009A17,_full_in- sert_sequence | −25.5 | −5.2 | 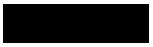 | −1.6 |
| AI464778_ESTs | −1.1 | −1.5 | −1.0 | −1.6 |
| AA674270_major_urinary_protein_1 | 1.0 | 1.0942˜ | 1.5 | −16.7 |
| AA175695_RIKEn_cDNA_5730455P16_gene | −1.6 | 1.25121˜ | −1.0 | −1.0 |
| AA675084_elastase_2 | −21.7 | −2.2 | 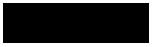 | −1.9 |
| AA555905_ribosomal_protein_L28 | 1.0 | 1.1 | 1.1 | −1.4 |
| AI120876_CD24a_antigen | −1.7 | −2.1 | 1.1 | −1.2 |
| AA414790_leukotriene_A4_hydrolase | −1.3 | −1.6 | −1.2 | −1.7 |
| AA821884_amylase_2,_pancreatic | −32.0 | −4.0 | 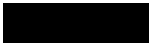 | −1.2 |
| AA185052_RIKEN_cDNA_2510004L01_gene | −1.0 | −1.8914˜ | 1.07838+ | 1.2 |
| AA733740_ESTs | −1.3 | −1.3 | −1.2 | −1.6 |
| W15809_hemoglobin,_beta_adult_major_chain | 1.3 | −3.7 | 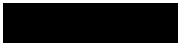 | −1.2 |
| AA122791_histocompatibility_2_Q_region_locus_7 | 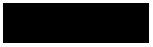 | −1.2 | −2.8 | −1.2 |
| AA106071_hemoglobin,_beta_adult_major_chain | −1.1 | −4.3 | 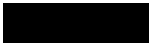 | 1.1 |
| W14953_kinesin-associated_protein_3 | −1.2 | −3.9332˜ | 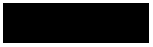 | −1.2 |
| AA727519_interferon-induced_protein_with_tetra- | 1.2 | 1.53884˜ | −1.2 | 1.2 |
| tricopeptide_repeats_3 | | | | |
| AI324398_glycosylphosphatidylinositol_specific_phospholipase_D1 | 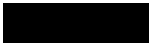 | −1.5 | 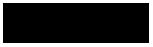 | −1.2 |
| AI326722_lymphocyte_antigen_6_complex,_locus_E | −1.1 | −2.0 | 1.1 | −1.1 |
| AA396056_ubiquitin_specific_protease_18 | 1.1 | −2.25683˜ | 1.2 | 1.1 |
| AA237793_rat_regenerating_islet-derived,_mouse_homolog_1 | −11.8 | −2.2 | 1.0 | −1.4 |
| AA066763_hemoglobin,_beta_adult_major_chain | 1.1 | −4.2 | 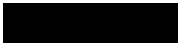 | −1.1 |
| AA796818_interferon-stimulated_protein_(15_kDa) | −1.0 | −1.5 | 1.2 | −1.2 |
| AA790398_ribonuclease_1,_pancreatic | −16.6 | −3.3 | 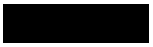 | −1.9 |
| AA175618_interferon-induced_protein_with_tetra- | −1.3 | −2.50237˜ | −1.96725+ | −1.2 |
| tricopeptide_repeats_1 | | | | |
| AA822105_major_urinary_protein_1 | −1.1 | 1.43224˜ | −1.3 | −71.1 |

STOMACH

| 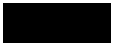 | blad | kidn | pl/ut | mam |
|---|---|---|---|---|
| Description | PL-9B_BDEnor | PL-10B_BDEnor | PL-11B_BDEnor | PL-12B_BDEnor |
| AA388685_ESTs | 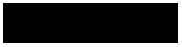 | 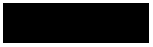 | 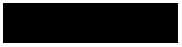 | 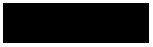 |
| AA517175_Mus_musculus_prion_inter- | 1.4 | 1.3 | 1.5 | 1.5 |
| actor_PINT1_mRNA,_partial_cds | | | | |
| AA522311_RIKEN_cDNA_0610038P07_gene | 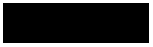 | 1.4 | 1.5 | 1.5 |
| AI430413_RIKEN_cDNA_1700020E22_gene | 1.3 | −1.2 | 1.1 | −1.0 |
| AA154791_calcitonin_receptor-like | 1.1 | −1.0 | 1.1 | 1.4 |
| AI552958_RIKEN_cDNA_1110003E08_gene | 1.1 | 1.1 | 1.2 | 1.2 |
| AI037647_Public_domain_EST | 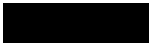 | 1.4 | 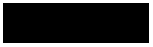 | 1.4 |
| AI466466_ESTs,_Weakly_similar_to_T27880_hypo- | 1.3 | 1.0 | 1.0 | 1.3 |
| thetical_protein_ZK520.1_-_Caenorhabditis_elegans_[C.elegans] | | | | |
| AA709728_DNA_segment,_Chr_3,_ERATO_Doi_330,_expressed | 1.5 | 1.3 | 1.5 | 1.2 |
| AA734324_ESTs | 1.4 | 1.1 | 1.3 | 1.4 |
| AA517891_ESTs,_Weakly_similar_to_E64778_probable_mem- | 1.2 | 1.1 | 1.2 | 1.5 |
| brane_protein_ybaL_-_Escherichia_coli_[E.coli] | | | | |
| AI509457_ESTs,_Highly_similar_to_KIAA0169_protein_[H.sapiens] | −1.1 | −1.0 | −1.0 | 1.1 |
| AA432613_ESTs,_Moderately_simi- | 1.3 | 1.1 | 1.2 | 1.2 |
| lar_to_1615347A_ras_p21_GTPase_activating_protein_[M.musculus] | | | | |
| AA079946_keratin_associated_protein_12-4 | 1.2 | 1.2 | 1.1 | 1.4 |
| AA245063_ESTs | 1.1 | 1.1 | 1.2 | 1.4 |
| AA396053_ESTs,_Moderately_similar_to_CYP2C40_[M.musculus] | 1.5 | 1.2 | 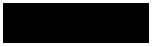 | 1.5 |
| AA154770_kinesin_family_member_C2 | 1.4 | 1.3 | 1.3 | 1.3 |
| AA020415_placental_specific_protein_1 | 1.2 | 1.0 | −1.4 | 1.3 |
| AA414213_upstream_binding_protein_1 | −1.4 | −1.3 | −1.3 | −1.2 |
| AA414750_abl-interactor_1 | −1.2 | −1.1 | −1.1 | −1.1 |
| AA763355_small_EDRK-rich_factor_2 | 1.0 | 1.2 | −1.2 | −1.2 |
| AA413684_RIKEN_cDNA_1300014E15_gene | −1.1 | −1.1 | 1.1 | 1.2 |
| AA414286_RIKEN_cDNA_1700016K02_gene | −1.1 | −1.2 | −1.2 | −1.3 |
| AA606943_glytathione_peroxidase_4 | −1.1 | 1.0 | −1.3 | −1.3 |
| AA268171_protein_phosphatase_2_(formerly_2A),_regula- | 1.1 | 1.2 | −1.1 | −1.1 |
| tory_subunit_A_(PR_65),_alpha_isoform | | | | |
| AA759679_la-associated_invariant_chain | −1.9 | −1.0 | −1.3 | −1.4 |
| W83121_legumain | −1.1 | 1.2 | −1.3 −1.1 | |
| AI594339_nuclear_factor_I/C | −1.3 | −1.2 | −1.2 | −1.4 |
| AA116994_promyelocytic_leukemia | −1.3 | −1.3 | −1.2 | −1.1 |
| AA671029_FK60_binding_protein_4_(59_kDa) | −1.1 | −1.1 | −1.4 | −1.2 |
| AA415514_microtubule- | 1.1 | −1.1 | 1.0 | −1.1 |
| associated_protein_,_RP/EB_family,_member_1 | | | | |
| AI036489_cyclin_G | −1.4 | −2.0 | −1.0 | −1.1 |
| AI048040_claudin_4 | −1.2 | −1.3 | −1.2 | −1.2 |
| AA791819_annexin_A5 | 1.1 | −1.1 | 1.0 | −1.1 |
| AA465997_RIKEN_cDNA_2010103A03_gene | 1.0 | −1.2 | 1.0 | −1.04125* |
| AA980349_glutathione_peroxidase_2,_pseudogene_1 | 1.1 | −1.1 | −1.5 | 1.5 |
| AA415520_RIKEN_cDNA_1810003N24_gene | 1.3 1.2 | 1.0 | −1.0 | |
| AI120979_cDNA_2410039E09_gene | −1.1 | −1.2 | −1.5 | −1.2 |
| AA106736_cytochrome_c_oxidase,_subunit_VIIa_3 | 1.0 | 1.0 | −1.2 | −1.1 |
| AA414844_emerin | −1.0 | 1.1 | −1.1 | −1.2 |
| AA716807_synaptophysin | −1.5 | −1.3 | −1.2 | −1.4 |
| AA154947_ESTs | −1.2 | −1.1 | −1.2 | −1.3 |
| W15971_FK506_binding_protein_2_(13_kDa) | −1.3 | −1.0 | −1.3 | −1.1 |
| AA051479_Public_domain_EST | −1.2 | −1.3 | −1.2 | −1.2 |
| AA008426_adaptor-related_protein_complex_AP- | −1.2 | −1.1 | −1.2 | −1.2 |
| 3,_sigma_2_subunit | | | | |
| W34862_glutathione_S-transferase,_mu_6 | 1.1 | 1.3 | −1.0 | 1.0 |
| AI322387_insulin-like_growth_factor_2 | −1.2 | −1.6 | −1.2 | −1.8 |
| AA727960_keratin_complex_1,_acidic,_gene_18 | 1.1 | −1.2 | −1.2 | 1.0 |
| AI892334_ribosomal_protein_L5 | 1.3 | −1.2 | −1.1 | −1.0 |
| AA529377_heat_shock_protein,_86_kDa_1 | 1.2 | −1.0 | −1.0 | 1.3 |
| AA871749_ESTs,_Weakly_simi- | −1.4 | −1.5 | −1.1 | −1.0 |
| lar_to_I49759_Hrs_-_[M.musculus] | −1.2 | −1.2 | −1.3 | −1.3 |
| AI645415_mini_chromosome_maintenance_deficient_(S._cererisiae) | | | | |
| AA415905_nucleolar_and_coiled-body_phosphoprotein_1 | −1.0 | 1.1 | −1.1 | −1.2 |
| AI428588_RIKEN_cDNA_3110039B05_gene | −1.4 | −1.1 | −1.4 | −1.4 |
| AA067069_histocompatibility_2,_T_region_locus_9 | −1.1 | 1.1 | −1.0 | −1.2 |
| AA674445_lectin,_galactose_binding,_soluble_9 | 1.3 | −1.2 | −1.0 | −1.0 |
| AA003005_CD151_antigen | −1.4 | −1.1 | −1.2 | −1.3 |
| AI181682_RIKEN_cDNA_3021401A05_gene | −1.2 | −1.4 | −1.0 | −1.0 |
| AI892472_RIKEN_cDNA_0610011D08_gene | 1.1 | −1.2 | −1.1 | −1.1 |
| AI385457_retinol_binding_protein_2,_cellular | −1.6 | −1.9 | −1.5 | −1.7 |
| AI049439_torsin_family_3,_member_A | −1.3 | −1.3 | −1.1 | −1.1 |
| [symbol] | 1.1 | 1.4 | 1.0 | −1.0 |
| AA549260_Public_domain_EST | 1.0 | −1.1 | 1.1 | 1.1 |
| AA097896_0-6-methylguanine-DNA_methyltransferase | −1.1 | −1.2 | −1.1 | −1.1 |
| AA824095_RIKEN_cDNA_2610205H19_gene | −1.1 | 1.0 | −1.4 | −1.4 |
| AA619950_cyclin_I | −1.3 | 1.1 | −1.1 | 1.1 |
| AA250087_proteosome_(prosome,_macropain)_sub- | −1.2 | 1.2 | −1.0 | −1.2 |
| unit,_beta_type_8_(large_multi-functionalₚᵣₒₜₑₐₛₑ_7) | | | | |
| AA030661_kinesin_light_chain_2 | −1.4 | −1.2 | −1.2 | −1.2 |
| AI536281_toricrin | 1.0 | 1.1 | 1.2 | 1.4 |
| W10007_mast_cell_protease_6 | −1.3 | −1.3 | −1.4 | −1.4/ |
| AA469667_tumor_rejection_antigen_gp96 | 1.2 | −1.1 | 1.1 | −1.0 |
| AA684319_protein_tyrosine_phosphatase_4a1 | 1.0 | −1.0 | 1.1 | −1.1 |
| AI553118_ESTs | −1.5 | −1.3 | −1.2 | 1.0 |
| AA500739_potassium_channel,_subfamily_K,_member_2 | −1.0 | −1.3 | −1.0 | −1.2 |
| AA600581_ribosomal_protein_L44 | −1.0 | −1.2 | −1.4 | −1.0 |
| AA684403_ESTs | −1.3 | −1.3 | −1.1 | −1.1 |
| AA690387_inactive_X_specific_transcripts | −1.5 | 1.2 | −1.3 | −1.2 |
| AA261440_lupus_nephiritis-associated_peptide_1 | −1.0 | 1.1 | −1.0 | −1.2 |
| W08321_inhibitor_of_DNA_bindind_1 | −1.0 | 1.0 | −1.0 | 1.2 |
| AA166386_transducer_of_ErbB-2.1 | −1.1 | −1.0 | −1.2 | 1.2 |
| AA437783_aplysia_ras-related_homolog_B_(RhoB) | −1.1 | −1.4 | −1.3 | −1.2 |
| AA067003_glutathione_S-transferase,_mu,_1 | 1.2 | 1.4 | −1.3 | −1.2 |
| AA066634_lactate_dehydrogenase_2,_B_chain | 1.3 | 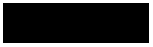 | 1.2 | −1.1 |
| AA606826_actin,_gamma,_cytoplasmic | 1.1 | 1.2 | 1.0 | −1.0 |
| AI121405_H2.0-like_homeo_box_gene | −1.5 | −1.3 | −1.3 | −1.3 |
| AA437457_nethionine_aminopeptidase | 1.0 | −1.0 | −1.0 | −1.1 |
| W17786_spinocerebellar_ataxia_10_homlog_(human) | 1.0 | 1.0 | −1.0 | −1.2 |
| AA619894_diazepam_binding_inhibitor | −1.0 | −1.1 | −1.2 | −1.0 |
| AA185212_ESTs | −1.2 | 1.1 | −1.2 | −1.3 |
| AI893237_RIKEN_cDNA_0610009D10_gene | −1.0 | −1.1 | −1.2 | −1.1 |
| AA674592_ESTs | −1.6 | −1.7 | −1.4 | −1.6 |
| AA420091_ESTs | −1.4 | −1.4 | −1.2 | −1.2 |
| AA874467_RIKEN_cDNA_1110035G07_gene | −1.5 | −1.3 | −1.1 | −1.2 |
| AA982254_RIKEN_cDNA_2210010C04_gene | −2.0 | −1.4 | −1.1 | −1.1 |
| AI552745_Mus_musculus,_clone_MGC:6888,_mRNA_com- | −1.2 | −1.4 | −1.3 | −1.1 |
| plete_cds | | | | |
| AA734030_opioid_receptor,_sigma_1 | −1.4 | −1.3 | −1.4 | −1.4 |
| AA920158_max_max_binding_protein | −1.4 | −1.1 | −1.1 | 1.0 |
| AA174675_ESTs | −1.6 | −1.5 | −1.5 | −1.6 |
| AA589092_ESTs | −1.4 | −1.6 | −1.3 | 1.1 |
| W13098_natriuretic_peptide_receptor_1 | −1.5 | −1.3 | −1.2 | −1.3 |
| AA210481_clusterin | 1.1 | 1.4 | −1.2 | 1.2 |
| AA062452_ESTs,_Moderately_similar_to_C211_HUMAN_PUTA- | −1.2 | 1.4 | −1.1 | 1.1 |
| TIVE_SURFACE_GLYCOPROTEIN_C21ORF1_PRE- | | | | |
| CURSOR_[H.sapiens] | | | | |
| AI390138_RIKEN_cDNA_3930401B19_gene | 1.5 | −1.9 | −1.3 | −1.1 |
| AA437424_Public_domain_EST | 1.1 | −1.3 | −1.0 | 1.2 |
| W36498_amelogenin | 1.0 | 1.1 | −1.2 | −1.0 |
| AI390970_RIKEN_cDNA_2310020F24_gene | −1.3 | −1.5 | −1.3 | −1.4 |
| AA437633_RIKEN_cDNA_2210401F17_gene | −1.1 | 1.1 | −1.2 | −1.2 |
| AA711419_POU_domain,_class_2,_transcription_factor_1 | −1.2 | −1.2 | −1.1 | −1.1 |
| AA138836_myxovirus_(influenza_virus)_resistance_1 | −1.1 | −1.1 | 1.1 | −1.8 |
| AA422762_ESTs | −1.3 | −1.2 | −1.2 | −1.2 |
| AA822609_ribosomal_protein_L27a | 1.1 | 1.0 | −1.2 | −1.0 |
| AA606665_Finkel-Biskis-Reilly_murine_sarcoma_virus_(FBR- | −1.1 | 1.0 | −1.3 | −1.0 |
| MuSV)_ubiquitously_expressed_(fox_derived) | | | | |
| AA465951_RIKEN_cDNA_9130022E05_gene | −1.4 | −1.3 | −1.3 | −1.2 |
| AA624488_prothymosin_alpha | −1.2 | −1.1 | −1.1 | −1.1 |
| AA771675_runt_related_transcription_factor_2 | −1.4 | −1.6 | −1.1 | −1.1 |
| W18330_tropomyosin_2,_beta | 1.1 | −1.1 | −1.3 | −1.4 |
| AI606650_calmodulin | 1.1 | −1.0 | −1.0 | −1.2 |
| AI894032_trefoil_factor_2_(spasmolytic_protein_1) | −1.4 | −1.9 | −1.0 | 1.4 |
| AA106338_Public_domain_EST | 1.2 | 1.2 | −1.1 | −1.0 |
| AI047049_ESTs | −1.3 | −1.2 | −1.3 | 1.0 |
| AA414427_RIKEN_cDNA_9430015P09_gene | 1.1 | −1.1 | 1.0 | −1.0 |
| AA175784_ESTs | −1.0 | 1.0 | 1.2 | 1.4 |
| AA414116_signal_recognition_particle_9_kDa | 1.0 | −1.1 | −1.2 | 1.0 |
| AA008228_peroxisomal_farnesylated_protein | −1.4 | −1.0 | −1.1 | −1.2 |
| AA444231_ribosomal_protein_S5 | 1.2 | 1.3 | −1.0 | −1.0 |
| AA199056_Public_domain_EST | −1.2 | −1.1 | −1.3 | −1.2 |
| AA619820_EST_AA536654 | 1.1 | 1.0 | −1.0 | −1.0 |
| AA471862_myosin_VI | 1.1 | 1.1 | −1.0 | 1.1 |
| AI536352_G_protein- | −1.3 | −1.1 | −1.1 | −1.0 |
| coupled_receptor,_family_C,_group_5,_member_B | | | | |
| AI604685_ubiquitin-like_(sentrin)_activating_enzyme_E1A | −1.4 | −1.4 | −1.3 | −1.2 |
| W14484_Public_domain_EST | −1.1 | 1.1 | −1.2 | −1.3 |
| AA065602_ribosomal_protein_L8 | 1.0 | 1.1 | −1.4 | −1.0 |
| AA596514_interferon_gamma_induced_GTPase | 1.0 | 1.2 | −1.2 | 1.1 |
| AI594147_beta-2_microglobulin | −1.0 | 1.3 | −1.1 | −1.2 |
| AI326556_FMs-like_tyrosine_kinase_1 | −1.3 | 1.2 | −1.6 | −1.5 |
| AI536309_RIKEN_cDNA_2210410L06_gene | −1.6 | −2.1 | −1.5 | −2.1 |
| AA067797_heat_shock_protein_cognate_70 | 1.5 | 1.3 | 1.0 | 1.2 |
| W57231_protein_kinase_C_and_casein_kinase_sub- | −1.3 | −1.2 | −1.3 | −1.2 |
| strate_in_neurons_2 | | | | |
| AI892534_ribosomal_protein_S24 | 1.2 | −1.4 | −1.1 | −1.2 |
| AA798766_selectin,_lymphocyte | −1.6 | −1.5 | −1.5 | −1.4 |
| AI182180_three_prime_repair_exonuclease_1 | −1.0 | 1.0 | −1.0 | −1.1 |
| AA107101_prostate_stem_cell_antigen | −1.0 | −1.1 | −1.4 | −1.4 |
| AA437941_glutathione_S-transferase,_pi_2 | 1.1 | 1.1 | −1.5 | −1.2 |
| AA276406_microsomal_triglyceride_transfer_protein | −1.3 | −1.3 | −1.4 | −1.3 |
| AA467239_RIKEN_cDNA_1700008C22_gene | −1.4 | −1.1 | −1.4 | −1.3 |
| AA065942_ATP_synthase,_H+_transporting_mitochon- | −1.0 | 1.1 | −1.2 | −1.1 |
| drial_F0_complex,_subunit_c_(subunit_9),_isoform_1 | | | | |
| W12636_proteaseome_(prosome,_macropain)_28_subunit,_3 | −1.1 | 1.0 | −1.1 | −1.3 |
| AA212150_eukaryotic_translation_elongation_factor_1_alpha_1 | 1.2 | 1.2 | −1.1 | 1.1 |
| AI892747_glytathione_S-transferase,_alpha_1_(Ya) | 1.4 | 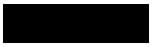 | −1.1 | 1.1 |
| AA667301_troponin_I,_cardiac | −2.2 | 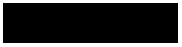 | −1.0 | 1.3 |
| AA072834_RIKEN_cDNA_9030418MN05_gene | −1.1 | 1.1 | 1.1 | 1.3 |
| AA422639_ESTs | −1.5 | −1.2 | −1.3 | −1.2 |
| AI549639_RIKEN_cDNA_2610301D06_gene | 1.0 | 1.1 | −1.2 | 1.1 |
| AA675111_RIKEN_cDNA_1810007A24_gene | −2.2 | −1.2 | −1.2 | −2.0 |
| AI323199_tumor_necrosis_factor_receptor_superfamily,_member_4 | −1.2 | −1.1 | −1.3 | −1.3 |
| AA727065_ESTs | −1.2 | −1.4 | −1.4 | −1.3 |
| AI536342_Public_domain_EST | 1.1 | 1.1 | 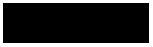 | 1.4 |
| AA066534_esterase_10 | 1.4 | −1. | −1.0 | −1.2 |
| AA674987_RIKEN_cDNA_1110038G14_gene | −1.4 | −1.4 | −1.8 | −1.2 |
| AA210505_Public_domain_EST | 1.3 | 1.1 | 1.1 | 1.2 |
| AA123007_2′-5′_oligoadenylate_synthetase-like | −1.1 | −1.1 | −1.1 | −1.1 |
| AA607044_RIKEN_cDNA_2700099C19_gene | −1.4 | −1.3 | −1.9 | |
| AA047991_RIKEN_cDNA_2700049I22_gene | −1.4 | −1.3 | −1.4 | −1.2 |
| W11185_ribosomal_protein_S15 | −1.3 | −1.1 | −1.2 | −1.2 |
| AI789568_potassium_voltage-gated_channel,_sub- | 1.09021+ | 1.1 | −1.01845+ | −1.5 |
| family_Q,_member_1 | | | | |
| AA437994_baculoviral_IAP_repeat-containing_4 | −1.7 | −1.6 | −1.3 | −1.3 |
| AA616180_RIKEN_cDNA_2010004B12_gene | −1.3 | −1.3 | −1.1 | −1.0 |
| W16358_mucin_1,_transmembrane | 1.1 | 1.1 | −1.2 | 1.0 |
| AA546625_ESTs,_Weakly_simi- | −1.7 | −1.7 | −1.4 | −1.5 |
| lar_to_SP62_MOUSE_SPLICEOSOME_ASSOCIATED_PRO- | | | | |
| TEIN_62_[M.musculus] | | | | |
| AA066685_villin_2 | −1.2 | 1.1 | −1.1 | −1.0 |
| AA674784_Mus_musculus_mosg_protein_(Mosg)_gene,_com- | −1.6 | −1.6 | −1.6 | −1.6 |
| plete_cds | | | | |
| AA450922_heat_shock_70kD_protein_5_(glucose- | 1.2 | −1.1 | −1.1 | −1.2 |
| regulated_protein,_78kD) | | | | |
| AA060105_trefoil_factor_1 | −1.6 | 1.0 | −1.0 | −1.5 |
| W29311_peptidylpropyl_isomerase_C-associated_protein | 1.3 | 1.1 | −1.1 | −1.1 |
| W53728_carbonyl_reductase_1 | −1.1 | 1.4 | −1.2 | −1.2 |
| AA066404_topoisomerase_(DNA)_II_beta | −1.2 | −1.5 | −1.3 | −1.2 |
| AA879946_RIKEN_cDNA_1110015M06_gene | −1.5 | −1.2 | −1.3 | −1.3 |
| AA667785_potassium_voltage-gated_channel,_Isk- | −1.5 | −1.2 | −1.4 | −1.5. |
| related_subfamily,_member_1 | | | | |
| AA437577_ESTs | −1.8 | −1.5 | −1.4 | −1.5 |
| AA260966_ATP_synthase,_H+_transporting,_mitochon- | −1.1 | −1.3 | −1.2 | −1.3 |
| drial_F0_complex,_subunit_g | | | | |
| AI551662_fos-like_antigen_2 | −1.4 | −1.4 | −1.4 | −1.5 |
| AA066282_small_inducible_cytokien_subfamily_B_(Cys- | −1.4 | −1.5 | −1.3 | −1.2 |
| X-Cys),_member_14 | | | | |
| AA426902_ESTs | −1.6 | −1.5 | −1.3 | −1.4 |
| AA106618_RIKEN_cDNA_0610007P06_gene | 1.2 | 1.1 | −1.1 | −1.0 |
| W62007_neuron_specific_gene_family_member_2 | −1.6 | −1.4 | −1.3 | −1.5 |
| AA051655_somatostalin | 1.1 | 1.0 | 1.3 | 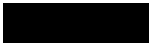 |
| AI020170_sema_domain,_immunoglobulin_domain_(Ig),_trans- | −1.5 | −1.1 | −1.2 | −1.0 |
| membrane_domain_(TM)_and_short_cyto- | | | | |
| plasmic_domain,_(semaphorin)_4D | | | | |
| AA770808_pericentriolar_material_1 | −1.6 | −1.5 | −1.6 | −1.6 |
| AA062293_cytochrome_c_oxidase,_subunit_Va | 1.1 | 1.0 | −1.1 | −1.0 |
| W36524_schafen_2 | −1.3 | −1.5 | −1.1 | −1.1 |
| AA106154_RIKEN_cDNA_1300003O09_gene | −1.1 | 1.1 | −1.1 | 1.1 |
| AI606037_Kruppel-like_factor_2_(lung) | −1.4 | −1.4 | −1.2 | −1.2 |
| AA049653_RAD51_like_3_(S._cerevisiae) | −1.5 | −1.2 | −1.3 | −1.3 |
| AA170469_interferon- | −1.1 | −1.2 | 1.1 | −1.1 |
| induced_protein_with_tetratricopeptide_repeats_1 | | | | |
| W12985_annexin_A10 | 1.1 | −1.0 | 1.0 | −1.9 |
| AA413907_ATP_synthase,_H+transporting,_mitochon- | −1.1 | 1.1 | −1.2 | −1.4 |
| drial_F1F0_complex,_subunit_e | | | | |
| AA14095_gene_rich_cluster,_C9_gene | −1.5 | −1.2 | −1.4 | −1.3 |
| AI876997_carboxyl_ester_lipase | −1.1 | 1.1 | 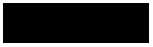 | 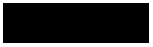 |
| AA444672_RIKEN_cDNA_2510001A17_gene | 1.2 | −1.4 | 1.0 | −1.2 |
| AA049003_heterogeneous_nuclear_ribonucleoprotein_D | −1.3 | −1.2 | −1.3 | −1.5 |
| AI325332_transglutaminase_3,_E_polypeptide | −1.7 | −1.4 | −1.5 | −1.7 |
| AA105866_glutathione_S-transferase,_alpha_4 | 1.3 | 1.3 | −1.1 | −1.2 |
| AA822098_aplipoprotein_A-I | −1.2 | −2.1 | −2.5 | −1.6 |
| AA619848_RIKEN_cDNA_4430402G14_gene | −1.1 | −1.1 | 1.0 | −1.1 |
| AA616014_ESTs | −1.5 | −1.4 | −1.5 | −1.3 |
| AI593751_ESTs,_Highly_similar_to_AF091457_1_zinc_fin- | −1.0 | −1.1 | 1.0 | −1.1 |
| ger_protein_RIN_ZF_[R.norvegicus] | | | | |
| AA718476_RIKEN_cDNA_0610025G13_gene | −1.0 | 1.2 | −1.3 | 1.2 |
| AA770709_aldehyde_dehydrogenase_family_3,_subfamily_A1 | −1.0 | 1.1 | 1.4 | 1.3 |
| AA437620_RIKEN_cDNA_1810030E05_gene | −1.5 | −1.3 | −1.3 | −1.2 |
| AA727627_ankyrin_1,_erythroid | −1.6 | −1.6 | −1.4 | −1.7 |
| AI451862_RIKEN_cDNA_0910001A18_gene | −2.4 | −1.3 | −1.1 | −1.3 |
| W15606_dynein,_axon,_heavy_chain_11 | −1.1 | −1.2 | −1.2 | −1.1 |
| AA437871_eukaryotic_translation_initiation_factor_4,_gamma_2 | 1.2 | 1.1 | −1.0 | 1.1 |
| AI893971_zinc_finger_protein_57 | −1.5 | −1.5 | −1.4 | −1.4 |
| AI594148_ribosomal_protein_S19 | −1.0 | −1.1 | −1.3 | −1.1 |
| AA048952_solute_carrier_family_4_(anion_exchanger),_member_2 | −1.3 | −1.0 | −1.2 | −1.0 |
| AI117832_thymidylate_kinase_family_LPS-inducible_member | 1.1 | 1.1 | 1.1 | 1.1 |
| AA444254_ornithine_decarboxylase,_structural | −1.4 | −1.1 | −1.1 | −1.0 |
| AI122517_interferon_regulatory_factor_7 | −1.1 | −1.1 | −1.1 | 1.0 |
| AI322733_2′-5′_oligodenylate_synthetase_1A | −1.2 | 1.1 | −1.2 | 1.1 |
| AA062440_quaking | 1.1 | −1.0 | −1.1 | −1.0 |
| AA245976_dihydrolipoamide_dehydrogenase | 1.2 | 1.2 | −1.1 | −1.1 |
| AI551576_ESTs | −1.2 | −1.3 | −1.0 | 1.1 |
| AA717025_Mus_musculus_10_day_old_male_pan- | −2.9 | 1.2 | −1.1 | −1.4 |
| crease_cDNA_full-length_enriched_library,_clone:1810009A17,_full_in- | | | | |
| sert_sequence | | | | |
| AI464778_ESTs | −1.7 | −1.6 | −1.5 | −1.6 |
| AA674270_major_urinary_protein_1 | −1.5 | −1.6 | −1.6 | −1.4 |
| AA175695_RIKEn_cDNA_5730455P16_gene | −1.2 | −1.1 | −1.0 | 1.3 |
| AA675084_elastase_2 | −4.0 | −1.4 | −1.2 | −1.4 |
| AA555905_ribosomal_protein_L28 | 1.0 | 1.3 | −1.3 | 1.0 |
| AI120876_CD24a_antigen | −1.0 | −1.0 | −1.4 | 1.1 |
| AA414790_leukotriene_A4_hydrolase | −2.0 | −1.7 | −1.6 | −1.8 |
| AA821884_amylase_2,_pancreatic | −4.1 | −1.1 | −1.1 | −1.2 |
| AA185052_RIKEN_cDNA_2510004L01_gene | 1.1 | −1.1 | 1.1 | 1.1 |
| AA733740_ESTs | −1.7 | −1.8 | −1.7 | −1.8 |
| W15809_hemoglobin,_beta_adult_major_chain | −1.0 | 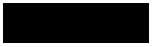 | −1.7 | −1.6 |
| AA122791_histocompatibility_2_Q_region_locus_7 | −1.3 | −1.0 | −1.3 | −1.2 |
| AA106071_hemoglobin,_beta_adult_major_chain | −1.3 | 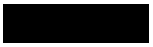 | −1.7 | −2.0 |
| W14953_kinesin-associated_protein_3 | −1.2 1.3 | −1.5 | −1.5 | |
| AA727519_interferon-induced_protein_with_tetra- | −1.0 | −1.3 | −1.1 | −1.2 |
| tricopeptide_repeats_3 | | | | |
| AI324398_glycosylphosphatidylinositol_specific_phospholipase_D1 | −1.3 | 1.2 | −1.6 | −1.6 |
| AI326722_lymphocyte_antigen_6_complex,_locus_E | −1.1 | −1.4 | 1.1 | −1.2 |
| AA396056_ubiquitin_specific_protease_18 | 1.1 | 1.0 | −1.1 | −1.2 |
| AA237793_rat_regenerating_islet-derived,_mouse_homolog_1 | −2.7 | −1.2 | −1.2 | −1.2 |
| AA066763_hemoglobin,_beta_adult_major_chain | −1.3 | 1.5 | −1.8 | −2.0 |
| AA796818_interferon-stimulated_protein_(15_kDa) | −1.1 | −1.2 | −1.1 | −1.2 |
| AA790398_ribonuclease_1,_pancreatic | −4.1 | −1.1 | −1.2 | −1.1 |
| AA175618_interferon-induced_protein_with_tetra- | −1.0 | −1.0 | 1.1 | 1.1 |
| tricopeptide_repeats_1 | | | | |
| AA822105_major_urinary_protein_1 | −1.6 | −2.2 | −1.1 | −1.2 |

LIVER

| 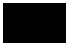 | spl | panc | 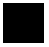 | stom |
|---|---|---|---|---|
| Description | PL-4B_BDEnor | PL-2B_BDEnor | PL-6B_BDEnor | PL-8B_BDEnor |
| 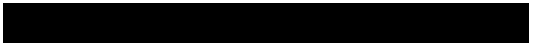 | −1.0 | −1.1 | 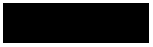 | −1.1 |
| 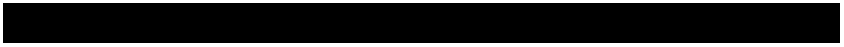 | 1.2 | −1.6 | 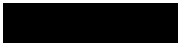 | −1.4 |
| 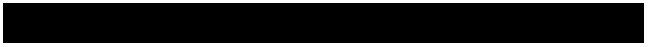 | 1.39696+ | 1.13053+ | 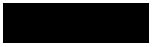 | 1.31173+ |
| 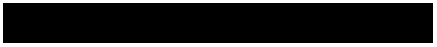 | 1.3 | −1.3 | 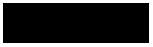 | 1.3 |
| 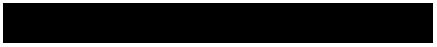 | 1.2 | −1.6 | 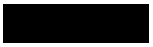 | 1.1 |
| 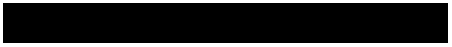 | 1.0 | −1.1 | 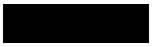 | 1.1 |
| 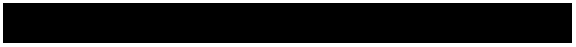 | 1.2 | −1.2 | 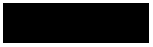 | 1.4 |
| 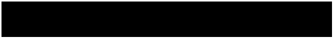 | 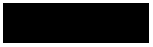 | −1.3 | 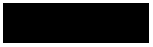 | 1.0 |
| 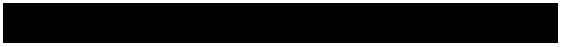 | −2.8 | 1.2 | 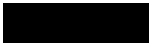 | 1.4 |
| 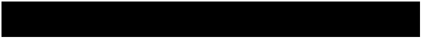 | 1.09601+ | 1.21261+ | 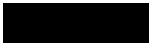 | 1.1 |
| 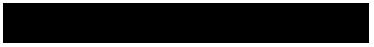 | 1.0 | 1.1 | 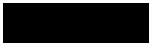 | 1.2 |
| 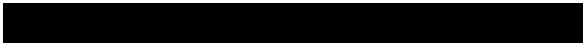 | 1.2 | −1.2 | 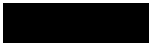 | 1.0 |
| 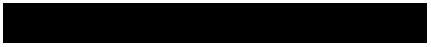 | 1.39098+ | −1.01923+ | 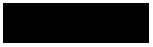 | 1.0093+ |
| 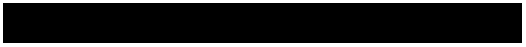 | −1.00156+ | −1.22244+ | 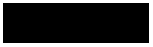 | 1.14256+ |
| 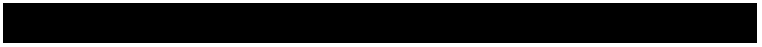 | 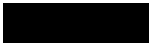 | −1.3 | 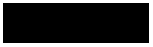 | −1.2 |
| 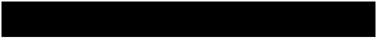 | 1.2 | −1.4 | 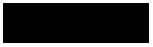 | 1.1 |
| 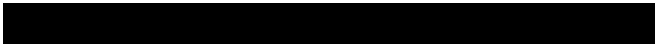 | 1.26667+ | −1.3006+ | 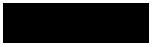 | 1.3 |
| 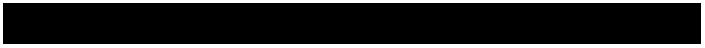 | 1.0 | −1.4 | 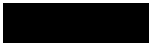 | 1.0 |
| AI323261_lipase,_hepatic | 1.13657+ | 1.08101+ | 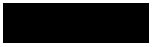 | −1.16185+ |
| AI047988_RIKEN_cDNA_2410041F14_gene | −1.2 | −1.059+ | 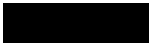 | −1.1 |
| AA929863_ESTs,_Weakly_similar_to_T46271_hypo- thetical_protein_DKFZp564P1263.1_[H.sapiens] | 1.1 | −1.1 | 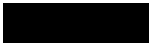 | 1.0 |
| AA244388_retinoic_acid_early_transcript_gamma | −1.1 | −1.05534+ | 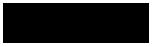 | 1.1 |
| AA771355_ficolin_A | 1.1 | −1.6 | 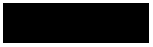 | 1.3 |
| W99034_ESTs | −1.2 | 1.3 | 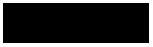 | 1.5 |
| AA822106_ESTs,_Weakly_similar_to_CO8A_HUMAN_COM- PLEMENT_COMPONENT_C8_AL- PHA_CHAIN_PRECURSOR_[H.sapiens] | 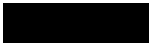 | −1.7 | 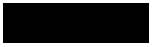 | −1.0 |
| AI117310_flavin_containing_monooxygenase_1 | 1.0 | −1.9 | 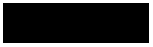 | −1.7 |
| AI789916_hydroxysteroid_11-beta_dehydrogenase_1 | 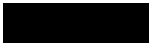 | 1.0 | 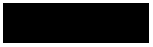 | −1.1 |
| AA106162_cytochrome_P450,_2c29 | 1.0 | −1.1 | 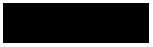 | 1.0 |
| AI597312_hydrooxysteroid_dehydrogenase_1,_delta<5>-3-beta | 1.2 | −1.5 | 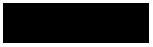 | −1.2 |
| AA261489_hypothetical_protein,_154 | 1.2 | 1.1 | 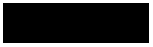 | 1.3 |
| W65070_ESTs | −1.2 | 1.4 | 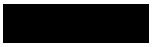 | 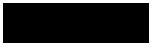 |
| W36474_metallothionine_2 | −1.2 | −2.2 | 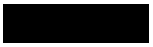 | 1.2 |
| AI386062_carbonic_anhydrase_3 | 1.1 | −1.3 | 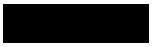 | −1.5 |
| AI509048_ATP-binding_cassette,a_sub- family_A_(ABC1),_member_8 | −1.03386+ | −1.25698+ | 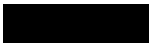 | −1.07194+ |
| AA674392_ESTs,_similar_to_A23772_LINE- 1_hypothetical_protein_-_mouse_[M.musculus] | −1.2 | −1.5 | 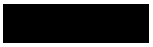 | 1.3 |
| AA260931_perosixomal_biogenesis_factor_11a | 1.3 | 1.33586+ | 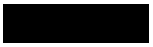 | 1.4 |
| AA822480_ESTs,_Weakly_similar_to_retinal_short- chain_dehydrogenase/reductase_retSDR1_[M.musculus] | 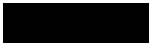 | 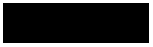 | 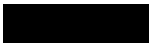 | 1.1 |
| AA108741_RIKEN_cDNA_0610025I19_gene | 1.1 | 1.0 | 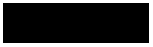 | −1.3 |
| AA572640_RIKEN_cDNA_1700124F02_gene | −1.10123+ | −1.00385+ | 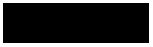 | −1.2 |
| AA712088_k-hydroxy-3-methylglutaryl- Coenzyme_A_reductase | −1.1 | −1.6 | 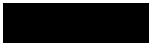 | −1.3 |
| AA272831_betaine-homocysteine_methyltransferase | 1.3 | −1.3 | 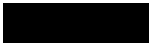 | −1.0 |
| AI386058_serum_albumin_variant | 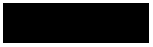 | −1.7 | 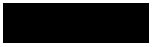 | 1.1 |
| W18463_thioether_S-methyltransferase | 1.0 | −1.5 | 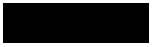 | −1.3 |
| AA867621_S-phase_kinase-associated_protein_2_(p45) | −1.8 | −1.2 | 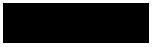 | 1.5 |
| AI893740_glycine_N-methyltransferase | 1.1 | 1.2 | −42 | 1.1 |
| AA420359_DNA_segment,_Chr_6,_ERATO_Doi,_109,_expressed | 1.1 | 1.1 | 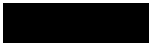 | −1.5 |
| AA116636_RIKEn_cDNA_1110013A05_gene | −1.5 | −1.0 | 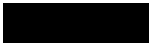 | 1.4 |
| AA511089_gluthione_S-transferase,_theta_2 | 1.4 | 1.2 | 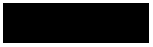 | −1.1 |
| AA024217_Public_domain_EST | −2.5 | 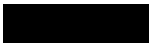 | 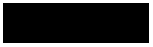 | 1.2 |
| AI325330_cytochrome_P450,_2b13,_phenobarbitol_in- ducible,_type_c | 1.4 | −1.1 | 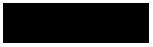 | 1.0 |
| AA415254_glutamate_oxaloacetate_transaminase_1,_soluble | 1.3 | 1.1 | 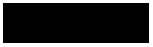 | −1.1 |
| AA237307_pyruvate_kinase_liver_and_red_blood_cell | −1.4 | −1.2 | 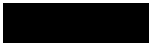 | −1.2 |
| AA822117_UDP-glucuronosyltransferase_1_family,_member_1 | −1.0 | −1.4 | 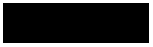 | −1.4 |
| W77429_selenium_binding_protein_1 | 1.0 | −1.4 | 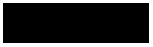 | −1.4 |
| [symbol] MINE_KINASE_[H.sapiens] | 1.4 | −1.0 | 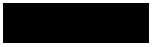 | −1.1 |
| AI892274_amine_N-sulfotransferase | −1.0 | −1.2 | 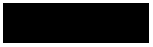 | −1.4 |
| AA067863_RIKEN_cDNA_2310057J18_gene | −1.2 | 1.2 | 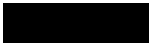 | −1.1 |
| AA437705_RIKEN_cDNA_2410012F02_gene | −1.2 | −1.0 | 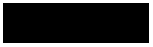 | −1.1 |
| AI551358_urate_oxidase | 1.15631+ | −1.05014+ | 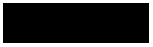 | 1.0088+ |
| AA822002_cytochrome_P450,_2c40 | 1.3 | −1.2 | 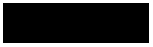 | 1.3 |
| AA674491_RIKEN_cDNA_1110007F05_gene | 1.3 | 1.14853+ | 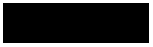 | −1.3 |
| AA656694_chtochrome_P450,_2b9,_phenobarbitol_in- ducible,_type_a | −1.1 | −1.4 | 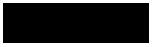 | 1.3 |
| AA545691_thymidine_kinase_1 | 1.2 | −1.2 | 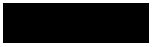 | −1.2 |
| [symbol] e_5,_clone_MGC:7544,_mRNA,_complete_cds | −1.2 | 1.0 | 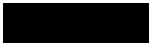 | 1.3 |
| AA260294_esterase_31 | 1.2 | −1.01604+ | 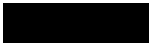 | 1.2 |
| AI120986_platelet_factor_4 | 1.1 | −1.01604+ | 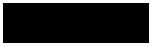 | 1.0 |
| AI326554_aldo-keto_reductase_family_1,_member_C1 | −1.0 | 1.0 | 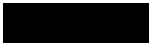 | 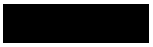 |
| AA061729_keratin_complex_1,_acidic,_gene_13 | 1.2 | −1.7 | −1.9 | 1.1 |
| AA028346_keratin_complex_1,_acidic,_gene_19 | −1.4 | −1.6 | −1.9 | −1.5 |
| AA008331_RIKEN_cDNA_2900055D14_gene | −1.4 | −1.1 | −1.9 | −1.2 |
| AI390445_ESTs | 1.1 | −1.81116+ | −1.9 | −1.6 |
| AA674784_Mus_musculus_mosg_protein_(Mosg)_gene,_com- | −1.6 | −1.9 | −1.9 | −2.2 |
| plete_cds | | | | |
| W53604_procollagen,_type_VI,_alpha_2 | −1.1 | −1.3 | −1.9 | −1.5 |
| AA607013_ESTs | −1.1 | 1.0 −1.9 | 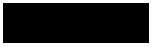 | |
| W62007_neuron_specific_gene_family_member_2 | −1.5 | −1.5 | −1.9 | −2.3 |
| AA794055_cysteine_rich_protein | 1.0 | 1.4 | −1.9 | −1.3 |
| AI593151_heparan_sulfate_2-O-sulfotransferase_1 | −1.2 | 1.0 | −1.9 | 1.4 |
| AA273820_schlafen_1 | −1.3 | −1.5 | −1.9 | −1.7 |
| AA619407_pancreatitis-associated_protein | 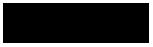 | 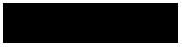 | −1.9 | 1.2 |
| AA867162_RIKEN_cDNA_4933400E14_gene | −1.3 | 1.1 | −1.9 | −1.4 |
| AI591831_ESTs | −1.5 | −1.4 | −2.0 | −1.8 |
| AI592552_phorbol-12-myristate-13-acetate-induced_protein_1 | −1.3 | −1.3 | −2.0 | −1.6 |
| AA871265_Public_domain_EST | 1.3 | −1.4 | −2.0 | 1.4 |
| AA445667_RIKEN_cDNA_4632401C08_gene | 1.3 | −1.2 | −2.0 | 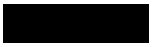 |
| AA591215_fatty_acid_binding_protein_5,_epidermal | 1.2 | −1.2 | −2.0 | −1.0 |
| AI594243_CD8beta_opposite_strand | −1.4 | −1.0 | −2.0 | −1.7 |
| W89253_insulin-like_growth_factor_binding_protein_5 | −1.0 | −1.3 | −2.0 | 1.2 |
| AA412831_Mus_musculus,_clone_MGC:6804,_mRNA_com- | −1.2 | −1.2 | −2.1 | −1.1 |
| plete_cds | | | | |
| AA726855_RIKEN_cDNA_3230402M22_gene | 1.3 | 1.5 | −2.1 | −1.5 |
| AI048040_claudin_4 | −1.6 | 1.5 | −2.1 | −1.9 |
| AI428626_ESTs | −1.4 | 1.1 | −2.1 | −1.8 |
| AA684403_ESTs | −1.4 | 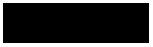 | −2.3 | −2.0 |
| AA450922_heat_shock_70kD_protein_5_(glucose- regulated_protein,_78kD) | 1.1 | 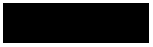 | −2.4 | −2.2 |

LIVER

| 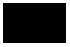 | int | col | br | lung |
|---|---|---|---|---|
| Description | PL-3B_BDEnor | PL-5B_BDEnor | PL-1B_BDEnor | PL-7B_BDEnor |
| 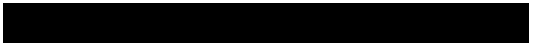 | 1.2 | −1.2 | −1.2 | 1.0 |
| 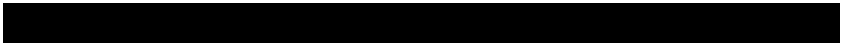 | −1.3 | −1.6 | −1.3 | −1.8 |
| 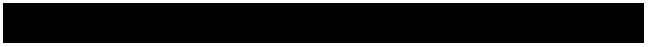 | 1.0 | 1.41016+˜ | 1.55264+ | −1.10611+ |
| 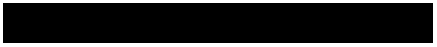 | 1.4 | 1.0 | −2.4 | 1.3 |
| 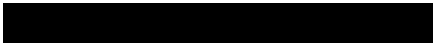 | 1.3 | 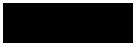 | −2.1 | 1.2 |
| 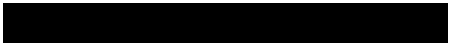 | 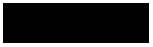 | 1.5 | −1.2 | −1.6 |
| 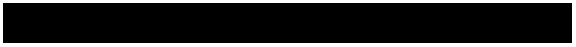 | −1.2 | 1.3 | −1.0 | −1.4 |
| 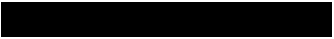 | 1.0 | −1.19636˜ | −1.7 | −1.7 |
| 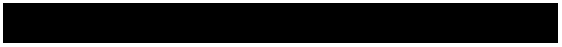 | 1.5 | 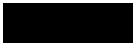 | 1.1 | −3.7 |
| 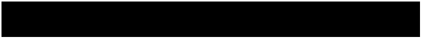 | 1.4 | 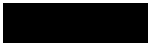 | −2.06124+ | 1.20289+ |
| 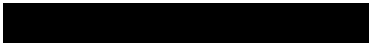 | 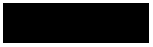 | 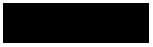 | 1.2 | −1.6 |
| 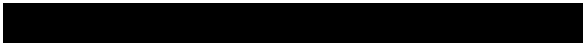 | 1.1 | 1.0 | 1.0 | 1.0 |
| 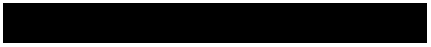 | −1.0149+ | −1.52171+˜ | −1.07118+ | 1.01954+ |
| 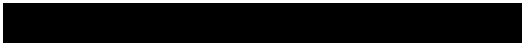 | −1.0782+ | −1.37095+˜ | 1.46647+ | 1.30476+ |
| 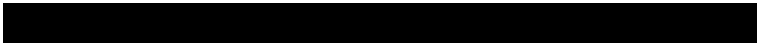 | 1.2 | −1.52324˜ | −1.5 | −1.3 |
| 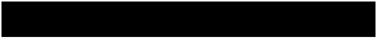 | 1.1 | 1.4 | −1.1 | 1.2 |
| 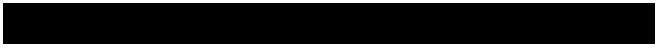 | 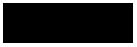 | −1.29025+˜ | −1.28348 + | −1.02353+ |
| 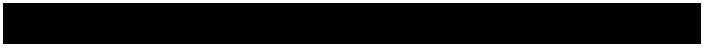 | −1.0 | −1.2 | −1.6 | −1.2 |
| AI323261_lipase,_hepatic | 1.08991+ | −1.06459+˜ | 1.52232+ | −1.53699+ |
| AI047988_RIKEN_cDNA_2410041F14_gene | −1.25687+ | 1.10888+˜ | −1.11193+03 | −1.52779+ |
| AA929863_ESTs,_Weakly_similar_to_T46271_hypo- thetical_protein_DKFZp564P1263.1_[H.sapiens] | −1.0 | 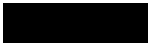 | −1.3 | 1.0 |
| AA244388_retinoic_acid_early_transcript_gamma | −1.2 | −1.00289+˜ | 1.21671+ | −1.0 |
| AA771355_ficolin_A | 1.2 | 1.0 | −1.1 | 1.1 |
| W99034_ESTs | 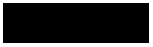 | 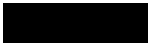 | 1.0 | −2.0 |
| AA822106_ESTs,_Weakly_similar_to_CO8A_HUMAN_COM- PLEMENT_COMPONENT_C8_AL- PHA_CHAIN_PRECURSOR_[H.sapiens] | 1.1 | 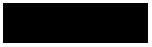 | −1.8 | −20.2 |
| AI117310_flavin_containing_monooxygenase_1 | −1.4 | −1.20807˜ | −1.2 | 1.0 |
| AI789916_hydroxysteroid_11-beta_dehydrogenase_1 | −1.3 | −1.26846˜ | 1.0 | −1.1 |
| AA106162_cytochrome_P450,_2c29 | 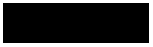 | 1.13279*˜ | −1.4 | −1.7 |
| AI597312_hydrooxysteroid_dehydrogenase_1,_delta<5>-3-beta | 1.3 | 1.51336˜ | 1.0 | −2.55267+** |
| AA261489_hypothetical_protein,_154 | 1.1 | 1.04783˜ | −1.1 | −1.2 |
| W65070_ESTs | 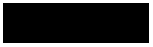 | 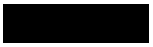 | 1.1 | −2.6 |
| W36474_metallothionine_2 | 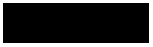 | 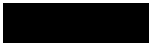 | 1.1 | −1.6 |
| AI386062_carbonic_anhydrase_3 | 1.1 | −2.4 | −1.7 | −2.1 |
| AI509048_ATP-binding_cassette,a_sub- | 1.38706+ | 1.0851+˜ | −1.15781+ | 1.4 |
| family_A_(ABC1),_member_8 | | | | |
| AA674392_ESTs,_similar_to_A23772_LINE- 1_hypothetical_protein_-_mouse_[M.musculus] | −1.3 | 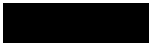 | −1.0 | −1.3 |
| AA260931_perosixomal_biogenesis_factor_11a | 1.4 | −1.02652+˜ | 1.16677+ | 1.5 |
| AA822480_ESTs,_Weakly_similar_to_retinal_short- chain_dehydrogenase/reductase_retSDR1_[M.musculus] | −1.2 | 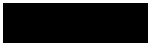 | −1.5 | −2.1 |
| AA108741_RIKEN_cDNA_0610025I19_gene | 1.3 | 1.0 | 1.2 | 1.2 |
| AA572640_RIKEN_cDNA_1700124F02_gene | 1.2 | 1.18494+˜ | 1.26699+ | −1.9 |
| AA712088_k-hydroxy-3-methylglutaryl- | −1.5 | −2.18263+˜ | −1.1 | −1.3 |
| Coenzyme_A_reductase | | | | |
| AA272831_betaine-homocysteine_methyltransferase | 1.1 | −1.62438˜ | 1.1 | −4.7 |
| AI386058_serum_albumin_variant | −1.1 | 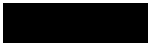 | −1.3 | −23.1 |
| W18463_thioether_S-methyltransferase | −1.6 | −1.6 | −1.9 | 1.0 |
| AA867621_S-phase_kinase-associated_protein_2_(p45) | 1.2 | 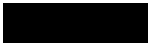 | −1.73102+03 | −2.2 |
| AI893740_glycine_N-methyltransferase | 1.3 | 1.37381˜ | −1.0 | −1.4 |
| AA420359_DNA_segment,_Chr_6,_ERATO_Doi,_109,_expressed | −1.3 | −1.1 | 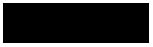 | 1.1 |
| AA116636_RIKEn_cDNA_1110013A05_gene | 1.2 | 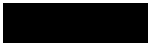 | −1.2 | −1.6 |
| AA511089_gluthione_S-transferase,_theta_2 | −1.1 | 1.0 | −1.4 | −1.3 |
| AA024217_Public_domain_EST | 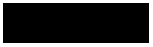 | 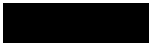 | −1.0 | −4.2 |
| AI325330_cytochrome_P450,_2b13,_phenobarbitol_in- ducible,_type_c | 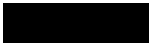 | 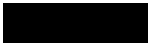 | 1.0 | 1.1 |
| AA415254_glutamate_oxaloacetate_transaminase_1,_soluble | 1.5 | 1.0 | 1.1 | 1.0 |
| AA237307_pyruvate_kinase_liver_and_red_blood_cell | 1.5 | 1.3 | −2.3 | −1.2 |
| AA822117_UDP-glucuronosyltransferase_1_family,_member_1 | 1.1 | 1.5 | −1.3 | −1.2 |
| W77429_selenium_binding_protein_1 | 1.1 | −1.8 | −1.4 | −1.2 |
| [symbol] | 1.2 | 1.10904˜ | −1.3 | −1.8 |
| MINE_KINASE_[H.sapiens] | | | | |
| AI892274_amine_N-sulfotransferase | −1.8 | 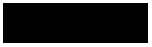 | −1.0 | −1.4 |
| AA067863_RIKEN_cDNA_2310057J18_gene | 1.2 | −1.58765˜ | 1.5 | 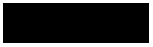 |
| AA437705_RIKEN_cDNA_2410012F02_gene | −1.4 | 1.19407˜ | −1.4 | −1.4 |
| AI551358_urate_oxidase | 1.10047+ | −1.60003+˜ | 1.3 | −2.5086+ |
| AA822002_cytochrome_P450,_2c40 | 1.4 | 1.4 | 1.1 | −3.9 |
| AA674491_RIKEN_cDNA_1110007F05_gene | 1.4 | −1.15618+˜ | 1.03774+ | 1.1 |
| AA656694_chtochrome_P450,_2b9,_phenobarbitol_in- ducible,_type_a | 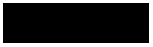 | 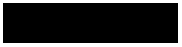 | −1.3 | 1.1 |
| AA545691_thymidine_kinase_1 | −1.1 | 1.24271˜ | −2.2 | −1.1 |
| [symbol] e_5,_clone_MGC:7544,_mRNA,_complete_cds | 1.1 | 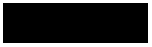 | −1.11126+ | −1.1 |
| AA260294_esterase_31 | 1.2 | −1.23474+˜ | −1.14543+ | −1.45171+ |
| AI120986_platelet_factor_4 | 1.2 | −2.4132+˜ | 1.23402+ | −1.1 |
| AI326554_aldo-keto_reductase_family_1,_member_C1 | 1.4 | 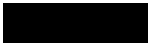 | 1.1 | −1.2 |
| AA061729_keratin_complex_1,_acidic,_gene_13 | 1.2 | 1.23314˜ | −1.7 | 1.5 |
| AA028346_keratin_complex_1,_acidic,_gene_19 | 1.0 | −1.2 | −2.3 | 1.1 |
| AA008331_RIKEN_cDNA_2900055D14_gene | 1.1 | −1.45209˜ | 1.3 | −1.0 |
| AI390445_ESTs | −1.4 | −1.16436+˜ | −1.50546+ | −1.0 |
| AA674784_Mus_musculus_mosg_protein_(Mosg)_gene,_com- | −1.0 | −1.1 | −1.2 | −1.4 |
| plete_cds | | | | |
| W53604_procollagen,_type_VI,_alpha_2 | −1.0 | 1.0 | −1.4 | −1.1 |
| AA607013_ESTs | 1.3 | −1.0562+03 ˜ | −1.0 | 1.2 |
| W62007_neuron_specific_gene_family_member_2 | −1.3 | −1.0 | 1.4 | −2.0 |
| AA794055_cysteine_rich_protein | 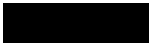 | −2.0 | 1.3 | 1.0 |
| AI593151_heparan_sulfate_2-O-sulfotransferase_1 | 1.3 | −1.34248+˜ | −1.0 | 1.2 |
| AA273820_schlafen_1 | −1.1 | −1.47898+˜ | 1.21692+ | −1.0 |
| AA619407_pancreatitis-associated_protein | 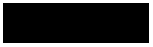 | −2.4 | −1.1 | −1.3 |
| AA867162_RIKEN_cDNA_4933400E14_gene | 1.3 | −1.81998+˜ | 1.5 | −1.0 |
| AI591831_ESTs | −1.1 | 1.0225+˜ | 1.37023+ | −1.23373+ |
| AI592552_phorbol-12-myristate-13-acetate-induced_protein_1 | −1.2 | −1.25542+˜ | −1.0 | −1.1 |
| AA871265_Public_domain_EST | −1.7 | −3.0 | −3.1 | −1.3 |
| AA445667_RIKEN_cDNA_4632401C08_gene | −1.0 | 1.1 | 1.3 | −1.2 |
| AA591215_fatty_acid_binding_protein_5,_epidermal | 1.2 | 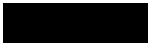 | 1.1 | 1.1 |
| AI594243_CD8beta_opposite_strand | 1.1 | −2.0 | 1.2 | −1.2 |
| W89253_insulin-like_growth_factor_binding_protein_5 | 1.4 | 1.16404˜ | 1.1 | 1.0 |
| AA412831_Mus_musculus,_clone_MGC:6804,_mRNA_com- | 1.0 | 1.07343˜ | −1.0 | −1.0 |
| plete_cds | | | | |
| AA726855_RIKEN_cDNA_3230402M22_gene | −1.7 | −1.4 | −1.4 | −1.2 |
| AI048040_claudin_4 | 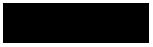 | −2.7 | 1.5 | −1.1 |
| AI428626_ESTs | 1.3 | −3.0 | 1.2 | −1.2 |
| AA684403_ESTs | 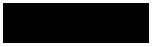 | −2.4 | 1.5 | −1.0 |
| AA450922_heat_shock_70kD_protein_5_(glucose- | −2.4 | −1.6 | 1.0 | −1.3 |
| regulated_protein,_78kD) | | | | |

LIVER

| 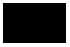 | blad | kidn | pl/ut | mam |
|---|---|---|---|---|
| Description | PL-9B_BDEnor | PL-10B_BDEnor | PL-11B_BDEnor | PL-12B_BDEnor |
| 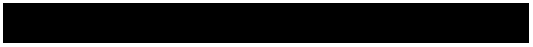 | −1.0 | 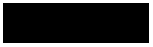 | −1.1 | 1.2 |
| 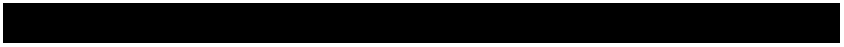 | −1.35 | −1.3 | 1.1 | −1.2 |
| 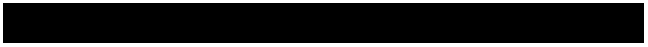 | −1.0531+ | 1.13851+ | −1.05399+ | −1.13526+ |
| 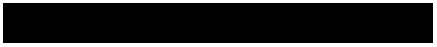 | 1.1 | 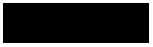 | 1.1 | 1.1 |
| 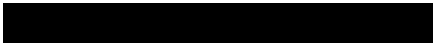 | −1.0 | 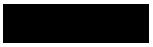 | −1.1 | −1.2 |
| 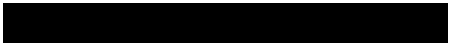 | 1.0 | 1.1 | −1.1 | 1.5 |
| 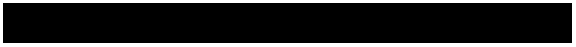 | 1.1 | 1.1 | −1.3 | −1.3 |
| 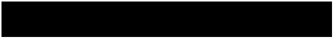 | 1.1 | 1.0 | 1.3 | 1.2 |
| 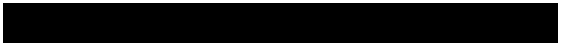 | −2.1 | −1.4 | −2.3 | −1.3 |
| 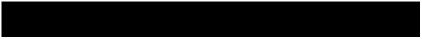 | 1.38263+ | 1.0 | 1.33503+ | 1.39713+ |
| 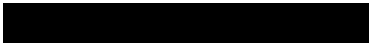 | −1.3 | 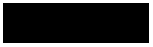 | −1.9 | −1.2 |
| 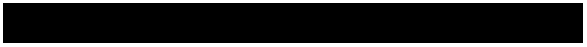 | −1.1 | 1.0 | 1.0 | 1.0 |
| 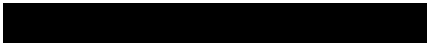 | 1.07526+ | 1.15311+ | 1.35956+ | 1.34636+ |
| 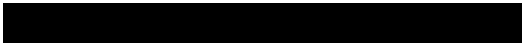 | 1.0216+ | 1.0616+ | 1.1 | −1.03458+ |
| 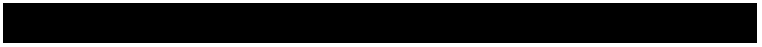 | 1.2 | 1.2 | −1.1 | 1.2 |
| 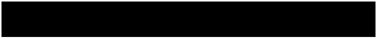 | −1.5 | 1.1 | −1.1 | −1.3 |
| 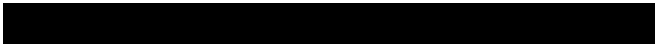 | 1.3 | 1.3 | −1.2 | 1.2 |
| 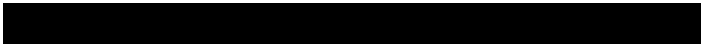 | −1.1 | 1.2 | 1.5 | −1.4 |
| AI323261_lipase,_hepatic | 1.1171+ | 1.19795+ | −1.11181+ | −1.1209+ |
| AI047988_RIKEN_cDNA_2410041F14_gene | −1.12343+ | −1.3 | −1.3 | −1.10113+ |
| AA929863_ESTs,_Weakly_similar_to_T46271_hypo- | −1.1 | −1.1 | −1.2 | −1.2 |
| thetical_protein_DKFZp564P1263.1_[H.sapiens] | | | | |
| AA244388_retinoic_acid_early_transcript_gamma | 1.1 | −1.2 | 1.1 | 1.1 |
| AA771355_ficolin_A | 1.2 | 1.1 | −1.1 | −1.1 |
| W99034_ESTs | −1.4 | −1.3 | −2.0 | 1.0 |
| AA822106_ESTs,_Weakly_similar_to_CO8A_HUMAN_COM- | −1.5 | −1.3 | −1.4 | −2.7 |
| PLEMENT_COMPONENT_C8_AL- | | | | |
| PHA_CHAIN_PRECURSOR_[H.sapiens] | | | | |
| AI117310_flavin_containing_monooxygenase_1 | −1.1 | 1.3 | −1.4 | −1.3 |
| AI789916_hydroxysteroid_11-beta_dehydrogenase_1 | 1.2 | −1.1 | −1.4 | −1.1 |
| AA106162_cytochrome_P450,_2c29 | −1.2 | 1.1 | −1.0 | −1.1 |
| AI597312_hydrooxysteroid_dehydrogenase_1,_delta<5>-3-beta | −1.2 | 1.2 | −1.5 | 1.1 |
| AA261489_hypothetical_protein,_154 | 1.0 | 1.1 | −1.0 | −1.1 |
| W65070_ESTs | −1.4 | −1.4 | −2.3 | −1.1 |
| W36474_metallothionine_2 | −1.1 | 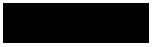 | −1.9 | 1.0 |
| AI386062_carbonic_anhydrase_3 | −1.3 | −1.2 | −1.6 | −1.5 |
| AI509048_ATP-binding_cassette,a_sub- | 1.3 | 1.0 | 1.36564+ | 1.4 |
| family_A_(ABC1),_member_8 | | | | |
| AA674392_ESTs,_similar_to_A23772_LINE- | −1.3 | −1.3 | 1.2 | −1.1 |
| 1_hypothetical_protein_-_mouse_[M.musculus] | | | | |
| AA260931_perosixomal_biogenesis_factor_11a | 1.1 | 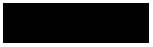 | 1.2 | 1.5 |
| AA822480_ESTs,_Weakly_similar_to_retinal_short- | 1.2 | −1.2 | 1.0 | −1.7 |
| chain_dehydrogenase/reductase_retSDR1_[M.musculus] | | | | |
| AA108741_RIKEN_cDNA_0610025I19_gene | 1.2 | 1.1 | 1.1 | 1.0 |
| AA572640_RIKEN_cDNA_1700124F02_gene | −1.3 | −1.0 | −1.2 | −1.2 |
| AA712088_k-hydroxy-3-methylglutaryl- | −1.0 | 1.3 | −1.2 | 1.0 |
| Coenzyme_A_reductase | | | | |
| AA272831_betaine-homocysteine_methyltransferase | 1.3 | −1.3 | 1.2 | −1.1 |
| AI386058_serum_albumin_variant | −1.4 | −1.5 | −1.3 | −2.9 |
| W18463_thioether_S-methyltransferase | −1.7 | 1.2 | −1.0 | −1.2 |
| AA867621_S-phase_kinase-associated_protein_2_(p45) | −1.8 | −1.4 | −1.9 | −1.3 |
| AI893740_glycine_N-methyltransferase | −1.1 | 1.0 | 1.2 | 1.4 |
| AA420359_DNA_segment,_Chr_6,_ERATO_Doi,_109,_expressed | 1.3 | 1.0 | 1.1 | −1.1 |
| AA116636_RIKEn_cDNA_1110013A05_gene | −1.3 | −1.3 | −1.8 | 1.1 |
| AA511089_gluthione_S-transferase,_theta_2 | 1.2 | 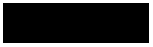 | 1.0 | 1.1 |
| AA024217_Public_domain_EST | −1.9 | −1.2 | −2.3 | 1.1 |
| AI325330_cytochrome_P450,_2b13,_phenobarbitol_in- | 1.2 | −1.2 | 1.1 | 1.4 |
| ducible,_type_c | | | | |
| AA415254_glutamate_oxaloacetate_transaminase_1,_soluble | 1.2 | 1.4 | 1.4 | 1.5 |
| AA237307_pyruvate_kinase_liver_and_red_blood_cell | 1.2 | −1.1 | 1.0 | 1.1 |
| AA822117_UDP-glucuronosyltransferase_1_family,_member_1 | 1.2 | 1.1 | −1.2 | −1.1 |
| W77429_selenium_binding_protein_1 | −1.0 | 1.1 | −1.1 | 1.1 |
| [symbol] | 1.1 | 1.2 | 1.2 | −1.1 |
| MINE_KINASE_[H.sapiens] | | | | |
| AI892274_amine_N-sulfotransferase | −1.1 | −1.2 | −1.4 | 1.2 |
| AA067863_RIKEN_cDNA_2310057J18_gene | 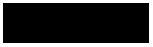 | 1.1 | −1.4 | −1.8 |
| AA437705_RIKEN_cDNA_2410012F02_gene | −1.2 | −1.1 | −1.2 | −1.0 |
| AI551358_urate_oxidase | 1.02179+ | −1.2 | 1.3 | 1.0 |
| AA822002_cytochrome_P450,_2c40 | −1.1 | −1.2 | −1.3 | −1.2 |
| AA674491_RIKEN_cDNA_1110007F05_gene | 1.1 | −1.0 | −1.1 | −1.2 |
| AA656694_chtochrome_P450,_2b9,_phenobarbitol_in- | 1.3 | 1.2 | 1.1 | 1.2 |
| ducible,_type_a | | | | |
| AA545691_thymidine_kinase_1 | 1.3 | 1.0 | −1.1 | 1.1 |
| [symbol] | −1.1 | 1.1 | −1.1 | −1.2 |
| e_5,_clone_MGC:7544,_mRNA,_complete_cds | | | | |
| AA260294_esterase_31 | −1.2 | −1.0 | 1.1 | 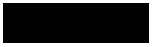 |
| AI120986_platelet_factor_4 | 1.2 | 1.2 | −1.1 | 1.1 |
| AI326554_aldo-keto_reductase_family_1,_member_C1 | 1.5 | 1.0 | 1.2 | 1.5 |
| AA061729_keratin_complex_1,_acidic,_gene_13 | −1.1 | −1.4 | 1.5 | 1.5 |
| AA028346_keratin_complex_1,_acidic,_gene_19 | −1.1 | −1.3 | −1.2 | −1.2 |
| AA008331_RIKEN_cDNA_2900055D14_gene | −1.2 | −1.2 | −1.2 | −1.1 |
| AI390445_ESTs | −1.1 | −1.2 | −1.1 | −1.1 |
| AA674784_Mus_musculus_mosg_protein_(Mosg)_gene,_com- | −1.6 | −1.6 | −1.6 | −1.6 |
| plete_cds | | | | |
| W53604_procollagen,_type_VI,_alpha_2 | 1.3 | −1.3 | −1.3 | −1.5 |
| AA607013_ESTs | −1.0 | 1.1 | 1.1 | 1.4 |
| W62007_neuron_specific_gene_family_member_2 | −1.6 | −1.4 | −1.3 | −1.5 |
| AA794055_cysteine_rich_protein | −1.4 | −1.0 | −1.3 | 1.1 |
| AI593151_heparan_sulfate_2-O-sulfotransferase_1 | −1.1 | −1.1 | 1.0 | −1.1 |
| AA273820_schlafen_1 | −1.4 | −1.1 | −1.3 | −1.0 |
| AA619407_pancreatitis-associated_protein | 1.0 | −1.2 | 1.3 | 1.1 |
| AA867162_RIKEN_cDNA_4933400E14_gene | −1.0 | −1.0 | −1.0 | −1.1 |
| AI591831_ESTs | −1.3 | −1.2 | −1.3 | −1.5 |
| AI592552_phorbol-12-myristate-13-acetate-induced_protein_1 | −1.2 | −1.4 | −1.2 | −1.1 |
| AA871265_Public_domain_EST | 1.2 | 1.3 | 1.4 | −1.7 |
| AA445667_RIKEN_cDNA_4632401C08_gene | 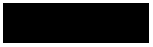 | 1.4 | 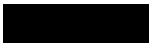 | 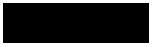 |
| AA591215_fatty_acid_binding_protein_5,_epidermal | 1.3 | 1.1 | −1.1 | −1.3 |
| AI594243_CD8beta_opposite_strand | −1.1 | −1.2 | −1.1 | −1.2 |
| W89253_insulin-like_growth_factor_binding_protein_5 | −1.0 | 1.1 | 1.0 | −1.6 |
| AA412831_Mus_musculus,_clone_MGC:6804,_mRNA_com- | −1.1 | −1.1 | −1.1 | −1.1 |
| plete_cds | | | | |
| AA726855_RIKEN_cDNA_3230402M22_gene | 1.4 | 1.2 | 1.1 | −1.3 |
| AI048040_claudin_4 | −1.2 | −1.3 | −1.2 | −1.2 |
| AI428626_ESTs | −1.1 | −1.3 | 1.0 | −1.2 |
| AA684403_ESTs | −1.3 | −1.3 | −1.1 | −1.1 |
| AA450922_heat_shock_70kD_protein_5_(glucose- | 1.2 | −1.1 | −1.1 | −1.2 |
| regulated_protein,_78kD) | | | | |

PANCREAS

| 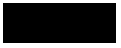 | spl | 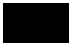 | liv | stom |
|---|---|---|---|---|
| Description | PL-4B_BDEnor | PL-2B_BDEnor | PL-6B_BDEnor | PL-8B_BDEnor |
| 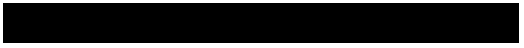 | 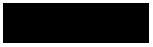 | 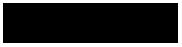 | −1.9 | 1.2 |
| 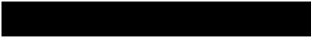 | 1.2 | 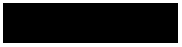 | 1.1 | −1.5 |
| 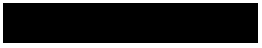 | 1.1 | 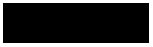 | 1.2 | −2.0 |
| 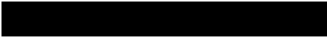 | −1.2 | 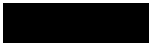 | 1.2 | 1.1 |
| AA790398_riboclease_1,_pancreatic | 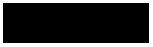 | 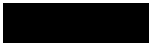 | −1.2 | −5.8 |
| AA686602_ESTs | −1.4 | 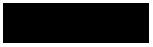 | 1.0 | −1.3 |
| AA681081_ESTs | −1.1 | 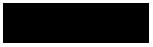 | −1.1 | 1.1 |
| AI597421_ESTs | 1.4 | 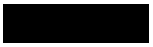 | 1.0 | 1.4 |
| AA600634_signal_sequence_receptor,_delta | 1.4 | 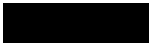 | 1.1 | −1.2 |
| AA458425_Swi/SNF_related_matrix_associated,_actin_de- pendent_regulator_of_chromatin,_subfamily_a-like_1 | 1.1 | 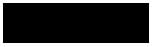 | −1.2 | 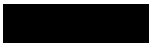 |
| AA571665_ESTs,_Moderately_similar_to_I38863_E1B_19K/Bcl-2- interacting_protein_Nip1_[H.sapiens] | 1.4 | 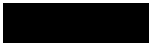 | 1.0 | 1.3 |
| AA223023_nescient_helix_loop_helix_1 | 1.4 | 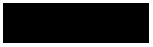 | 1.0 | 1.3 |
| [symbol] e | −1.0 | 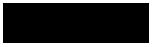 | 1.1 | −1.9 |
| AI120332_RIKEN_cDNA_1810055O16_gene | 1.3 | 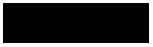 | −1.2 | −1.2 |
| AA469630_choline_kinase | 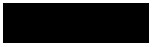 | 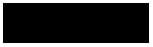 | −1.4 | 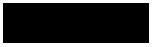 |
| AA763351_death-associated_kinase_3 | 1.2 | 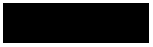 | −1.2 | −1.1 |
| AA543968_RIKEN_cDNA_2810418N01_gene | 1.2 | 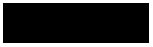 | −1.1 | −1.6 |
| AI549624_RIKEN_cDNA_0610041E09_gene | 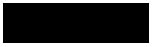 | 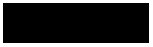 | −1.3 | 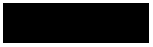 |
| AA162226_Public_domain_EST | 1.4 | 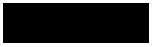 | 1.0 | 1.5 |
| AI643201_Mus_musculus_TOB3_mRNA,_complete_cds | 1.3 | 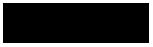 | 1.1 | 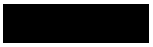 |
| AI597097_Public_domain_EST | 1.3 | 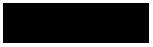 | −1.1 | −1.0 |
| AA607983_zinc_finger_protein_101 | 1.23774+ | 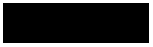 | −1.38204+ | 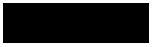 |
| AA667785_potassium_voltage-gated_channel,_Isk- | −1.8 | −1.9 | −1.3 | −2.2 |
| related_subfamily,_member_1 | | | | |
| AI152562_proliferin | 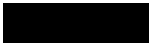 | −1.9 | 1.0 | −1.2 |
| AA547393_ESTs | 1.0 | −1.8 | −1.3 | 1.1 |
| AA435278_celsequestrin_1 | −1.0 | −1.9 | −1.2 | −1.3 |
| AA795319_myosin_light_chain,_phosphorylatable,_cardiac_ventricles | −1.1 | −1.9 | −1.2 | −1.4 |
| AA674784_Mus_musculus_mosg_protein_(Mosg)_gene,_com- | −1.6 | −1.9 | −1.9 | −2.2 |
| pletely_cds | | | | |
| AA546660_calpain_10 | 1.1 | −1.9 | 1.4 | −1.2 |
| AI606520_ESTs | 1.1 | −1.9 | −1.1 | −1.1 |
| AI604245_protocadherin_7 | −1.8 | −1.9 | −1.5 | −1.8 |
| AA672330_ESTs | −1.4 | −1.9 | −1.1 | −1.3 |
| AA008228_peroxisomal_farnesylated_protein | −1.4 | −1.9 | −1.3 | −2.1 |
| W36635_carbonyl_reductase_2 | 1.2 | −1.9 | −1.4 | 1.4 |
| AI596699_ESTs | −1.5 | −1.9 | −1.5 | −1.6 |
| AI117310_flavin_containing_monooxygenase_1 | 1.0 | −1.9 | 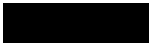 | −1.7 |
| AA822027_ATPase_Cu++_transporting,_beta_polypeptide | 1.2 | −1.9 | 1.2 | −1.4 |
| AA789551_ESTs,_Weakly_simi- | −1.1 | −1.9 | −1.3 | 1.0 |
| lar_to_I49441_Mouse_19.5_mRNA,_com- | | | | |
| plete_cds_-_mnouse_[M.musculus] | | | | |
| AI551573_ESTs | −1.5 | −1.9 | −1.4 | −1.6 |
| AA684426_ESTs | 1.0 | −1.9 | −1.2 | 1.1 |
| AA028578_keratin_complex_2,_basic,_gene_4 | 1.2 | −1.9 | −1.3 | −1.0 |
| W29933_growth_arrest_and_DNA_-damage- | 1.2 | −2.0 | −1.3 | −1.1 |
| inducible_45_gamma | | | | |
| AI325332_transglutaminase_3,_E_polypeptide | −2.0 | −2.0 | −1.4 | −2.3 |
| AA727521_Mus_musculus_11_kDa_secreted_protein_pre- cursor,_mRNA,_complete_cds | 1.1 | −2.0 | −1.4 | 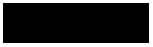 |
| AA220024_trophoblast_specific_protein | 1.5 | −2.0 | −1.2 | 1.5 |
| AA089188_uromodulin | −1.2 | −2.0 | −1.0 | 1.1 |
| AA623704_Public_domain_EST | 1.2 | −2.0 | −1.0 | −1.0 |
| AA726162_keratin_complex_1,_acidic,_gene_16 | 1.1 | −2.0 | 1.2 | −1.1 |
| AI551662_fos-like_antigen_2 | −2.1 | −2.0 | −1.4 | −2.2 |
| W83106_ELAV_(embryonic_lethal,_abnormal_vision,_Drosophila)- | −1.5 | −2.0 | −1.7 | −1.8 |
| like_3_(Hu_antigen_C) | | | | |
| AI481911_Mus_musculus,_clone_clone_MGC:6727,_mRNA,_com- | −1.1 | −2.0 | −1.8 | 1.0 |
| plete_cds | | | | |
| AA794176_ESTs,_Moderately_similar_to_SPA- | −1.0 | −2.0 | −1.2 | −1.2 |
| 1_like_protein_p1294_[R.norvegicus] | | | | |
| AA693177_ESTs | 1.4 | −2.0 | 1.0 | 1.1 |
| AA770808_pericentriolar_material_1 | −1.9 | −2.1 | −1.6 | −2.3 |
| AA727627_ankyrin_1,_erythroid | −1.8 | −2.1 | −1.6 | −2.5 |
| AA717019_ATPase,_Ca++_transporting,_car- | −1.2 | −2.1 | −1.1 | −1.5 |
| diac_muscle,_fast_twitch_1 | | | | |
| AA684191_CDC-like_kinase | 1.5 | −2.1 | −1.3 | −1.1 |
| W54403_ATPase,_Cu++_transporting,_beta_polypeptide | 1.2 | −2.1 | 1.1 | −1.0 |
| AA245952_cyclin-dependent_kinase-like_2_(CDC2-related_kinase) | −1.1 | −2.1 | −1.7 | −1.8 |
| AI385457_retinol_binding_protein_2,_cellular | −2.0 | −2.1 | −1.6 | −1.9 |
| AA733740_ESTs | −2.0 | −2.2 | −1.6 | −2.9 |
| AA138265_RIKEN_cDNA_2010309G21_gene | −1.1 | −2.2 | −1.5 | −1.2 |
| AA152636_integrin_alpha_4 | 1.2 | −2.2 | −1.3 | 1.1 |
| W36474_metallothionein_2 | −1.2 | −2.2 | 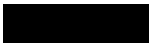 | 1.2 |
| AA4147090_leukotriene_A4_hydrolase | −2.1 | −2.2 | −1.6 | −2.8 |
| AI664286_tumor_necrosis_factor_receptor_superfamily,_member_9 | 1.3 | −2.3 | −1.1 | −1.3 |
| AA822105_major_urinary_protein_1 | −1.1 | −2.4 | −28.7649* | −7.8 |
| AA821997_myoglobin | −1.0 | −2.5 | 1.0 | 1.1 |
| AA822257_myosin_light_chain,_alkali,_fast_skeletal_muscle | 1.4 | −2.5 | −1.2 | −1.1 |
| AA600571_intracisternal_A_particles | 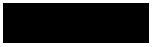 | −3.1 | −1.1 | 1.1 |
| AA444538_ESTs,_Weakly_similar_to_MDP1_MICRO- | −2.1 | −3.9 | 1.2 | −1.4 |
| SOMAL_DIPEPTIDE_PRECURSOR_[M.musculus] | | | | |
| W34262_heat_shock_protein,_25_kDa | −1.2 | −5.8 | 1.1 | −1.8 |
| AI390138_RIKEN_cDNA_3930401B19_gene | −1.4 | −10.7 | −1.1 | −2.0 |

PANCREAS

| 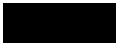 | int | col | br | lung |
|---|---|---|---|---|
| Description | PL-5B_BDEnor | PL-5B_BDEnor | PL-1B_BDEnor | PL-7B_BDEnor |
| 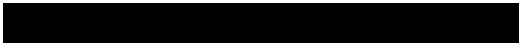 | 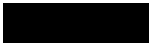 | −2.4 | −1.1 | −1.3 |
| 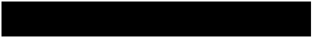 | −1.8 | 1.4 | 1.4 | −1.4 |
| 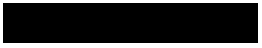 | −1.8 | −1.4 | 1.3 | −1.1 |
| 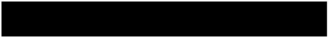 | 1.4 | −1.3 | 1.1 | −1.2 |
| AA790398_riboclease_1,_pancreatic | −16.6 | −3.3 | 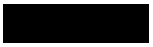 | −1.9 |
| AA686602_ESTs | 1.2 | −2.4 | 1.3 | −1.6 |
| AA681081_ESTs | 1.1 | −1.6 | 1.3 | −1.4 |
| AI597421_ESTs | 1.2 | −1.0 | 1.0 | 1.5 |
| AA600634_signal_sequence_receptor,_delta | −1.0 | −1.2 | −1.1 | −1.2 |
| AA458425_Swi/SNF_related_matrix_associated,_actin_de- | 1.4 | 1.09081+˜ | 1.4 | 1.5 |
| pendent_regulator_of_chromatin,_subfamily_a-like_1 | | | | |
| AA571665_ESTs,_Moderately_similar_to_I38863_E1B_19K/Bcl-2- | 1.2 | −1.0 | 1.1 | 1.4 |
| interacting_protein_Nip1_[H.sapiens] | | | | |
| AA223023_nescient_helix_loop_helix_1 | 1.2 | −1.1 | 1.2 | 1.3 |
| [symbol] | 1.1 | −1.2 | 1.2 | 1.0 |
| e | | | | |
| AI120332_RIKEN_cDNA_1810055O16_gene | 1.1 | −1.7 | 1.2 | −1.2 |
| AA469630_choline_kinase | 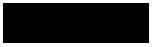 | 1.31548+˜ | 1.3 | 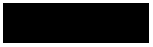 |
| AA763351_death-associated_kinase_3 | −1.0 | −1.6 | 1.3 | −1.1 |
| AA543968_RIKEN_cDNA_2810418N01_gene | 1.2 | −1.1 | 1.1 | −1.2 |
| AI549624_RIKEN_cDNA_0610041E09_gene | 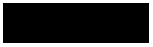 | −1.01549+˜ | 1.1 | 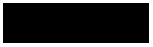 |
| AA162226_Public_domain_EST | 1.2 | 1.1 | 1.2 | 1.4 |
| AI643201_Mus_musculus_TOB3_mRNA,_complete_cds | 1.5 | 1.20006+˜ | 1.25034+ | 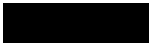 |
| AI597097_Public_domain_EST | −1.6 | 1.2 | 1.2 | −1.0 |
| AA607983_zinc_finger_protein_101 | 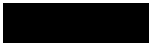 | 1.1254+˜ | 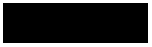 | 1.432+ |
| AA667785_potassium_voltage-gated_channel,_Isk- | −1.2 | −1.0 | −1.3 | −1.4 |
| related_subfamily,_member_1 | | | | |
| AI152562_proliferin | −1.3 | −1.24501˜ | −1.2 | −1.2 |
| AA547393_ESTs | −1.5 | 1.2 | 1.13657* | 1.3 |
| AA435278_celsequestrin_1 | −1.3 | 1.0767˜ | −1.8 | −1.3 |
| AA795319_myosin_light_chain,_phosphorylatable,_cardiac_ventricles | −1.3 | 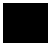 | −3.0 | −1.8 |
| AA674784_Mus_musculus_mosg_protein_(Mosg)_gene,_com- | −1.0 | −1.1 | −1.2 | −1.4 |
| pletely_cds | | | | |
| AA546660_calpain_10 | 1.0 | −1.15946+˜ | 1.1 | 1.0 |
| AI606520_ESTs | −1.7 | 1.1 | 1.0 | 1.2 |
| AI604245_protocadherin_7 | −1.0 | −1.46876˜ | −1.1 | −1.2 |
| AA672330_ESTs | −1.8 | 1.0 | −1.1 | −1.1 |
| AA008228_peroxisomal_farnesylated_protein | 1.0 | −1.1 | −1.2 | −1.2 |
| W36635_carbonyl_reductase_2 | 1.0 | 1.3 | −2.3 | 1.1 |
| AI596699_ESTs | −1.3 | −1.43833˜ | −1.03626+ | −1.1 |
| AI117310_flavin_containing_monooxygenase_1 | −1.4 | −1.20807˜ | −1.2 | 1.0 |
| AA822027_ATPase_Cu++_transporting,_beta_polypeptide | −1.0 | 1.17594˜ | −1.2 | −21.9 |
| AA789551_ESTs,_Weakly_simi- | 1.2 | −1.2 | −1.3 | 1.1 |
| lar_to_I49441_Mouse_19.5_mRNA,_com- | | | | |
| plete_cds_-_mnouse_[M.musculus] | | | | |
| AI551573_ESTs | −1.4 | −1.48844˜ | −1.7 | −1.5 |
| AA684426_ESTs | −1.6 | 1.0 | 1.2 | 1.1 |
| AA028578_keratin_complex_2,_basic,_gene_4 | −1.0 | 1.44671˜ | −2.1 | 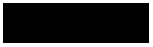 |
| W29933_growth_arrest_and_DNA_-damage- inducible_45_gamma | 1.0 | 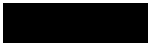 | −1.3 | −1.7 |
| AI325332_transglutaminase_3,_E_polypeptide | −1.2 | −1.3 | −1.2 | −1.2 |
| AA727521_Mus_musculus_11_kDa_secreted_protein_pre- cursor,_mRNA,_complete_cds | 1.5 | 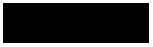 | −1.6 | 1.2 |
| AA220024_trophoblast_specific_protein | 1.1 | −1.0 | −2.2 | −1.2 |
| AA089188_uromodulin | −1.3 | 1.0 | −4.0 | −1.4 |
| AA623704_Public_domain_EST | −1.7 | 1.2 | −1.0 | 1.3 |
| AA726162_keratin_complex_1,_acidic,_gene_16 | −1.0 | 1.3 | −1.5 | −1.2 |
| AI551662_fos-like_antigen_2 | −1.2 | −1.1 | −1.3 | −1.4 |
| W83106_ELAV_(embryonic_lethal,_abnormal_vision,_Drosophila)- | −1.2 | −1.44607˜ | 1.0 | −1.2 |
| like_3_(Hu_antigen_C) | | | | |
| AI481911_Mus_musculus,_clone_clone_MGC:6727,_mRNA,_com- | −1.8 | −2.3 | −1.5 | −2.4 |
| plete_cds | | | | |
| AA794176_ESTs,_Moderately_similar_to_SPA- | −2.0 | 1.2 | 1.2 | 1.3 |
| 1_like_protein_p1294_[R.norvegicus] | | | | |
| AA693177_ESTs | −1.8 | 1.2 | 1.3 | 1.3 |
| AA770808_pericentriolar_material_1 | −1.1 | −1.2 | −1.0 | −1.5 |
| AA727627_ankyrin_1,_erythroid | −1.3 | −1.2 | −1.4 | −1.4 |
| AA717019_ATPase,_Ca++_transporting,_car- | −1.3 | 1.2 | −2.8 | −1.4 |
| diac_muscle,_fast_twitch_1 | | | | |
| AA684191_CDC-like_kinase | −2.0 | −1.4 | −1.3 | 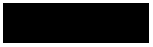 |
| W54403_ATPase,_Cu++_transporting,_beta_polypeptide | 1.2 | 1.3 | −2.9 | −7.9 |
| AA245952_cyclin-dependent_kinase-like_2_(CDC2-related_kinase) | −1.2 | −1.38223˜ | −1.2 | 1.1 |
| AI385457_retinol_binding_protein_2,_cellular | 1.5 | 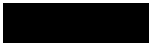 | −1.3 | −1.9 |
| AA733740_ESTs | −1.3 | −1.3 | −1.2 | −1.6 |
| AA138265_RIKEN_cDNA_2010309G21_gene | −1.0 | −1.9 | −1.1 | 1.1 |
| AA152636_integrin_alpha_4 | −2.2 | 1.1 | −1.0 | 1.3 |
| W36474_metallothionein_2 | 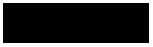 | 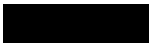 | 1.1 | −1.6 |
| AA4147090_leukotriene_A4_hydrolase | −1.3 | −1.6 | −1.2 | −1.7 |
| AI664286_tumor_necrosis_factor_receptor_superfamily,_member_9 | −1.3 | 1.2 | −2.7 | −1.3 |
| AA822105_major_urinary_protein_1 | −1.1 | 1.43224˜ | −1.3 | −71.1 |
| AA821997_myoglobin | 1.0 | −1.2 | −1.8 | −1.7 |
| AA822257_myosin_light_chain,_alkali,_fast_skeletal_muscle | −1.1 | 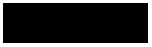 | −1.3 | 1.0 |
| AA600571_intracisternal_A_particles | −1.4 | 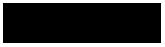 | −1.2 | 1.4 |
| AA444538_ESTs,_Weakly_similar_to_MDP1_MICRO- SOMAL_DIPEPTIDE_PRECURSOR_[M.musculus] | −1.1 | −1.19886+˜ | 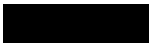 | 1.0 |
| W34262_heat_shock_protein,_25_kDa | 1.1 | −1.0 | −1.0 | −1.1 |
| AI390138_RIKEN_cDNA_3930401B19_gene | −1.2 | −2.1 | −1.3 | −1.2 |

PANCREAS

| 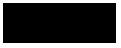 | blad | kidn | pl/ut | mam |
|---|---|---|---|---|
| Description | PL-9B_BDEnor | PL-10B_BDEnor | PL-11B_BDEnor | PL-12B_BDEnor |
| 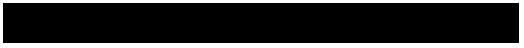 | 1.0 | −1.2 | 1.3 | 1.1 |
| 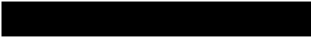 | 1.1 | 1.1 | −1.6 | 1.2 |
| 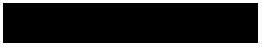 | 1.1 | 1.4 | −1.2 | 1.2 |
| 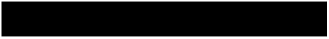 | −1.3 | 1.1 | −1.1 | 1.1 |
| AA790398_riboclease_1,_pancreatic | −4.1 | −1.1 | −1.2 | −1.1 |
| AA686602_ESTs | −1.5 | −1.2 | −1.3 | −1.1 |
| AA681081_ESTs | −1.3 | 1.0 | −1.2 | −1.1 |
| AI597421_ESTs | 1.3 | 1.4 | 1.3 | 1.2 |
| AA600634_signal_sequence_receptor,_delta | 1.1 | 1.1 | −1.1 | 1.1 |
| AA458425_Swi/SNF_related_matrix_associated,_actin_de- pendent_regulator_of_chromatin,_subfamily_a-like_1 | 1.3 | 1.3 | 1.3 | 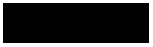 |
| AA571665_ESTs,_Moderately_similar_to_I38863_E1B_19K/Bcl-2- | 1.4 | 1.2 | 1.4 | 1.4 |
| interacting_protein_Nip1_[H.sapiens] | | | | |
| AA223023_nescient_helix_loop_helix_1 | 1.2 | 1.3 1.4 | 1.3 | |
| [symbol] | 1.1 | 1.4 | 1.0 | −1.0 |
| e | | | | |
| AI120332_RIKEN_cDNA_1810055O16_gene | 1.1 | 1.0 | −1.2 | 1.0 |
| AA469630_choline_kinase | 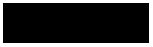 | 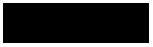 | 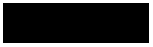 | 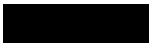 |
| AA763351_death-associated_kinase_3 | 1.0 | 1.1 | −1.2 | 1.0 |
| AA543968_RIKEN_cDNA_2810418N01_gene | −1.0 | −1.1 | −1.2 | −1.2 |
| AI549624_RIKEN_cDNA_0610041E09_gene | 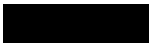 | 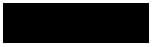 | 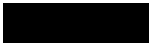 | 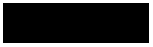 |
| AA162226_Public_domain_EST | 1.5 | 1.2 | 1.4 | 1.3 |
| AI643201_Mus_musculus_TOB3_mRNA,_complete_cds | 1.4 | 1.5 | 1.5 | 1.4 |
| AI597097_Public_domain_EST | 1.2 | −1.9 | 1.0 | −1.3 |
| AA607983_zinc_finger_protein_101 | 1.5 | 1.2 | 1.4 | 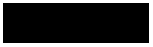 |
| AA667785_potassium_voltage-gated_channel,_Isk- | −1.5 | −1.2 | −1.4 | −1.5 |
| related_subfamily,_member_1 | | | | |
| AI152562_proliferin | −1.1 | −1.4 | −1.1 | 1.2 |
| AA547393_ESTs | −1.1 | −1.2 | 1.2 | −1.1 |
| AA435278_celsequestrin_1 | −1.6 | 1.0 | 1.1 | −1.4 |
| AA795319_myosin_light_chain,_phosphorylatable,_cardiac_ventricles | −1.3 | 1.1 | 1.1 | 1.0 |
| AA674784_Mus_musculus_mosg_protein_(Mosg)_gene,_com- | −1.6 | −1.6 | −1.6 | −1.6 |
| pletely_cds | | | | |
| AA546660_calpain_10 | −1.1 | −1.2 | −1.1 | −1.0 |
| AI606520_ESTs | −1.1 | −1.2 | −1.1 | −1.1 |
| AI604245_protocadherin_7 | −1.4 | −1.2 | −1.3 | −1.5 |
| AA672330_ESTs | −1.3 | −1.3 | −1.1 | −1.0 |
| AA008228_peroxisomal_farnesylated_protein | −1.4 | −1.0 | −1.1 | −1.2 |
| W36635_carbonyl_reductase_2 | 1.0 | 1.2 | 1.5 | 1.1 |
| AI596699_ESTs | −1.6 | −1.4 | −1.2 | 1.1 |
| AI117310_flavin_containing_monooxygenase_1 | −1.1 | 1.3 | −1.4 | −1.3 |
| AA822027_ATPase_Cu++_transporting,_beta_polypeptide | −1.5 | −1.5 | −1.8 | −1.7 |
| AA789551_ESTs,_Weakly_simi- | −1.1 | 1.1 | 1.2 | −1.0 |
| lar_to_I49441_Mouse_19.5_mRNA,_com- | | | | |
| plete_cds_-_mnouse_[M.musculus] | | | | |
| AI551573_ESTs | −1.7 | −1.3 | −1.4 | −1.2 |
| AA684426_ESTs | −1.1 | −1.3 | 1.2 | −1.1 |
| AA028578_keratin_complex_2,_basic,_gene_4 | 1.4 | −1.1 | 1.5 | 1.3 |
| W29933_growth_arrest_and_DNA_-damage- | 1.3 | −1.1 | −1.3 | 1.4 |
| inducible_45_gamma | | | | |
| AI325332_transglutaminase_3,_E_polypeptide | −1.7 | −1.4 | −1.5 | −1.7 |
| AA727521_Mus_musculus_11_kDa_secreted_protein_pre- | 1.1 | 1.1 | 1.5 | 1.2 |
| cursor,_mRNA,_complete_cds | | | | |
| AA220024_trophoblast_specific_protein | 1.4 | 1.1 | 1.2 | 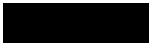 |
| AA089188_uromodulin | −1.7 | 1.1 | −1.2 | −1.4 |
| AA623704_Public_domain_EST | −1.0 | −1.2 | 1.2 | −1.1 |
| AA726162_keratin_complex_1,_acidic,_gene_16 | 1.3 | −1.2 | −1.1 | −1.1 |
| AI551662_fos-like_antigen_2 | −1.4 | −1.4 | −1.4 | −1.5 |
| W83106_ELAV_(embryonic_lethal,_abnormal_vision,_Drosophila)- | −1.6 | −1.3 | −1.3 | −1.3 |
| like_3_(Hu_antigen_C) | | | | |
| AI481911_Mus_musculus,_clone_clone_MGC:6727,_mRNA,_com- plete_cds | −1.4 | 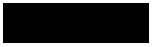 | −1.4 | −1.4 |
| AA794176_ESTs,_Moderately_similar_to_SPA- | −1.3 | −1.3 | 1.1 | −1.1 |
| 1_like_protein_p1294_[R.norvegicus] | | | | |
| AA693177_ESTs | 1.0 | −1.3 | 1.4 | −1.1 |
| AA770808_pericentriolar_material_1 | −1.6 | −1.5 | −1.6 | −1.6 |
| AA727627_ankyrin_1,_erythroid | −1.6 | −1.6 | −1.4 | −1.7 |
| AA717019_ATPase,_Ca++_transporting,_car- | −1.8 | −1.2 | 1.1 | −1.1 |
| diac_muscle,_fast_twitch_1 | | | | |
| AA684191_CDC-like_kinase | 1.4 | −2.1 | 1.4 | 1.2 |
| W54403_ATPase,_Cu++_transporting,_beta_polypeptide | 1.1 | −1.1 | −1.1 | 1.2 |
| AA245952_cyclin-dependent_kinase-like_2_(CDC2-related_kinase) | −1.0 | −1.3 | −1.1 | 1.1 |
| AI385457_retinol_binding_protein_2,_cellular | −1.6 | −1.9 | −1.5 | −1.7 |
| AA733740_ESTs | −1.7 | −1.8 | −1.7 | −1.8 |
| AA138265_RIKEN_cDNA_2010309G21_gene | −1.3 | −1.0 | −1.0 | −2.4 |
| AA152636_integrin_alpha_4 | −1.3 | −1.4 | 1.2 | −1.0 |
| W36474_metallothionein_2 | −1.1 | 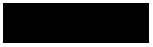 | −1.9 | 1.0 |
| AA4147090_leukotriene_A4_hydrolase | −2.0 | −1.7 | −1.6 | −1.8 |
| AI664286_tumor_necrosis_factor_receptor_superfamily,_member_9 −1.1 | −1.2 | 1.1 | 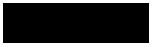 | |
| AA822105_major_urinary_protein_1 | −1.6 | −2.2 | −1.1 | −1.2 |
| AA821997_myoglobin | −4.3 | 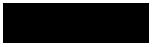 | −1.1 | −1.7 |
| AA822257_myosin_light_chain,_alkali,_fast_skeletal_muscle | −1.6 | −1.4 | −1.2 | −1.6 |
| AA600571_intracisternal_A_particles | 1.1 | −1.7 | 1.5 | 1.3 |
| AA444538_ESTs,_Weakly_similar_to_MDP1_MICRO- | 1.3 | −1.3 | −1.3 | 1.3 |
| SOMAL_DIPEPTIDE_PRECURSOR_[M.musculus] | | | | |
| W34262_heat_shock_protein,_25_kDa | 1.5 | −1.3 | −1.1 | −1.1 |
| AI390138_RIKEN_cDNA_3930401B19_gene | 1.5 | −1.9 | −1.3 | −1.1 |

SPLEEN

| 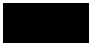 | 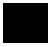 | panc | liv | stom |
|---|---|---|---|---|
| Description | PL-4B_BDEnor | PL-2B_BDEnor | PL-6B_BDEnor | PL-8B_BDEnor |
| 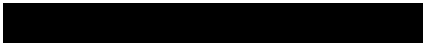 | 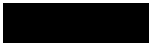 | 1.1 | −1.2 | −2.8 |
| 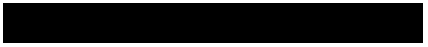 | 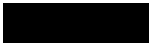 | −1.7 | 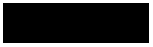 | 1.1 |
| 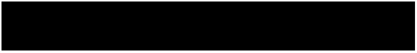 | 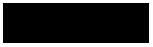 | −1.7 | 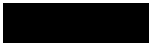 | −1.0 |
| 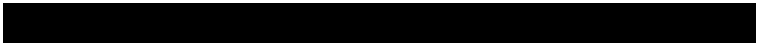 | 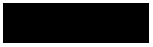 | 1.4 | −1.3 | −3.9 |
| AA982254_RIKEN_cDNA_2210010C04_gene | 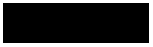 | 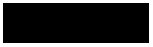 | −1.3 | −2.0 |
| AA675084_elastase_2 | 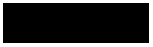 | 1.4 | −1.2 | −2.7 |
| AA674409_pancreatic_lipase_related_protein_1 | 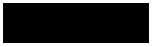 | 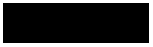 | −1.4 | −1.7 |
| AA222208_surfactant_associated_protein_C | 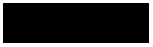 | −1.7 | −1.3 | −1.4 |
| AA445731_heat_shock_protein_cognate_70,_testis | 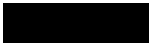 | 1.1 | 1.0 | −1.2 |
| AA717025_Mus_musculus_10_day_old_male_pan- creas_cDNA_RIKEN_RIKEN_full- length_enriched_library,_clone:1810009A17,_full_insert_sequence | 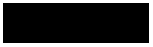 | −1.1 | −1.2 | −2.7 |
| AI510089_ESTs | 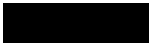 | 1.5 | 1.5 | 1.5 |
| W53916_RIKEN_cDNA_3300001P10_gene | 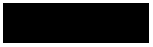 | 1.5 | 1.0 | 1.1 |
| AA238294_sema_domain,_immunoglobulin_domain_(Ig),_trans- | −1.9 | −1.6 | 1.1 | −1.7 |
| membrane_domain_(TM)_and_short_cyto- | | | | |
| plasmic_domain,_(semaphorin)_G | | | | |
| AA437577_ESTs | −1.9 | −1.8 | −1.5 | −2.2 |
| AA426902_ESTs | −1.9 | −1.3 | −1.4 | −2.3 |
| AA770808_pericentriolar_material_1 | −1.9 | −2.1 | −1.6 | −2.3 |
| AA684320_activin_receptor_interacting_protein_1 | −1.9 | 1.4 | 1.3 | 1.3 |
| AA871914_defensin_related_cryptidin_16 | −1.9 | −1.3 | −1.1 | 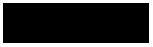 |
| AA546625_ESTs,_Weakly_simi- | −1.9 | −1.6 | −1.4 | −2.2 |
| lar_to_SP62_MOUSE_SPLICESOME_AS- | | | | |
| SOCIATED_PROTEIN_62_[M.musculus] | | | | |
| AI464778_ESTs | −1.9 | −1.6 | −1.6 | −2.7 |
| AA822098_apolipoprotein_A-I | −1.9 | −1.5 | 1.1 | −2.4 |
| AI325332_transglutaminase_3,_E_polypeptide | −2.0 | −2.0 | −1.4 | −2.3 |
| AA822366_inhibitor_of_DNA_binding_4 | −2.0 | 1.3 | 1.2 | 1.5 |
| AA733740_ESTs | −2.0 | −2.2 | −1.6 | −2.9 |
| AA881492_ESTs | −2.0 | 1.3 | 1.1 | −1.2 |
| AI608086_runt_related_transcription_factor_3 | −2.0 | 1.1 | 1.2 | 1.4 |
| AI121690_RIKEN_cDNA_1300007C21_gene | −2.0 | −1.7 | −1.4 | −1.4 |
| AI385457_retinol_binding_protein_2,_cellular | −2.0 | −2.1 | −1.6 | −1.9 |
| AA255025_S100_calcium-binding_protein_A9_(calgranulin_B) | −2.1 | −1.3 | −1.2 | 1.1 |
| AI551662_fos-like_antigen_2 | −2.1 | −2.0 | −1.4 | −2.2 |
| AA414790_leukotriene_A4_hydrolase | −2.1 | −2.2 | −1.6 | −2.8 |
| AA014727_tumor-suppressing_subchromosomal_transferable_frag- ment_3 | −2.1 | 1.1 | 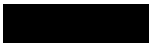 | −1.2 |
| AA546645_cell_division_cycle_2_homolog_(S._prombe)-like_2 | −2.1 | −1.1 | 1.3 | 1.4 |
| AA444538_ESTs,_Weakly_similar_to_MDP1_MOUSE_MICRO- | −2.1 | −3.9 | 1.2 | −1.4 |
| SOMAL_DIPEPTIDE_PRECURSOR_[M.musculus] | | | | |
| AA608466_ESTs | −2.2 | −1.2 | 1.4 | 1.1 |
| AI386257_calbindin-D9K | −2.2 | −1.3 | 1.2 | 1.5 |
| AA087193_lipocalin_2 | −2.2 | −1.7 | −1.8 | −1.3 |
| AA034678_rhotekin | −2.3 | 1.0 | 1.2 | −1.0 |
| AA414653_ESTs,_Weakly_similar_to_KIAA0542_protein_[H.sapiens] | −2.4 | −1.13914+ | −1.5 | 1.1 |
| AA771229_ESTs,_Weakly_similar_to_zinc_finger_pro- | −2.4 | 1.2 | 1.3 | 1.2 |
| tein_95_[M.musculus] | | | | |
| AA982151_proteinase_3 | −2.4 | 1.1 | 1.2 | −1.1 |
| AA176045_forkhead_box_C2 | −2.5 | 1.1 | 1.4 | 1.4 |
| AA024217_Public_domain_EST | −2.5 | 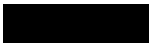 | 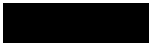 | 1.2 |
| AI428661_ESTs | −2.6 | −1.1 | 1.0 | −1.4 |
| AA874101_tumor-associated_calcium_signal_transducer_2 | −2.6 | −1.1 | 1.5 | 1.3 |
| AA474336_synaptonemal_complex_protein_3 | −2.8 | 1.2 | 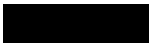 | 1.4 |
| AA031015_myloperoxidase | −2.9 | −1.1 | 1.0 | −1.1 |
| AA189266_ESTs | −3.2 | 1.4 | 1.5 | 1.5 |
| AA060282_neutrophilic_granule_protein | −4.0 | 1.39257+ | 1.2 | −1.0 |

SPLEEN

| 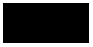 | int | col | br | lung | |
|---|---|---|---|---|---|
| Description | PL-3B_BDEnor | PL-5B_BDEnor | PL-1B_BDEnor | PL-7B_BDEnor | |
| 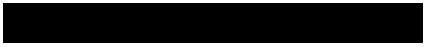 | −32.0 | −4.0 | 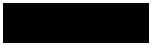 | −1.2 | |
| 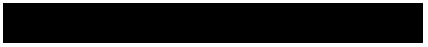 | −1.1 | 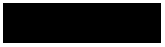 | −1.3 | −23.1 | |
| 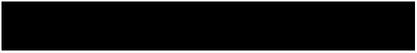 | 1.1 | 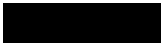 | −1.8 | −20.2 | |
| 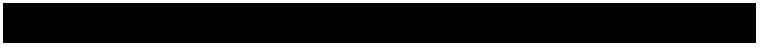 | −11.8 | −2.2 | 1.0 | −1.4 | |
| AA982254_RIKEN_cDNA_2210010C04_gene | −9.5 | −1.3 | 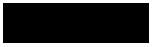 | −1.5 | |
| AA675084_elastase_2 | −21.7 | −2.2 | 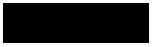 | −1.9 | |
| AA674409_pancreatic_lipase_related_protein_1 | −5.9 | −1.0 | 1.2 | 1.0 | |
| AA222208_surfactant_associated_protein_C | 1.2 | 1.23582˜ | −1.6 | −1.1 | |
| AA445731_heat_shock_protein_cognate_70,_testis | −1.1 | −1.3 | −1.8 | 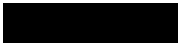 | |
| AA717025_Mus_musculus_10_day_old_male_pan- creas_cDNA_RIKEN_RIKEN_full- length_enriched_library,_clone:1810009A17,_full_insert_sequence | −25.5 | −5.2 | 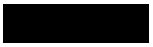 | −1.6 | |
| AI510089_ESTs | 1.3 | −1.0161˜ | 1.2 | 1.4 | |
| W53916_RIKEN_cDNA_3300001P10_gene | 1.3 | −1.69676˜ | −1.2 | 1.1 | |
| AA238294_sema_domain,_immunoglobulin_domain_(Ig),_trans- | −1.4 | −1.3 | −1.2 | −1.4 | |
| membrane_domain_(TM)_and_short_cyto- | | | | | |
| plasmic_domain,_(semaphorin)_G | | | | | |
| AA437577_ESTs | −1.2 | −1.47043˜ | −1.53147+ | −1.5 | |
| AA426902_ESTs | 1.0 | −1.4 | −1.1 | −1.3 | |
| AA770808_pericentriolar_material_1 | −1.1 | −1.2 | −1.0 | −1.5 | |
| AA684320_activin_receptor_interacting_protein_1 | 1.5 | 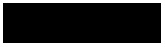 | 1.0 | −2.7 | |
| AA871914_defensin_related_cryptidin_16 | 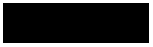 | 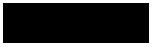 | 1.1 | −1.2 | |
| AA546625_ESTs,_Weakly_simi- | −1.2 | −1.4 | 1.2 | −1.2 | |
| lar_to_SP62_MOUSE_SPLICESOME_AS- | | | | | |
| SOCIATED_PROTEIN_62_[M.musculus] | | | | | |
| AI464778_ESTs | −1.1 | −1.5 | −1.0 | −1.6 | |
| AA822098_apolipoprotein_A-I | 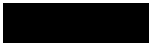 | 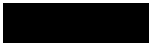 | −1.9 | −9.0 | |
| AI325332_transglutaminase_3,_E_polypeptide | −1.2 | −1.3 | −1.2 | −1.2 | |
| AA822366_inhibitor_of_DNA_binding_4 | 1.4 | 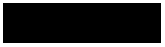 | 1.1 | −2.1 | |
| AA733740_ESTs | −1.3 | −1.3 | −1.2 | −1.6 | |
| AA881492_ESTs | 1.1 | 1.1 | 1.0 | −2.4 | |
| AI608086_runt_related_transcription_factor_3 | 1.4 | 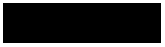 | −1.75763+ | −2.0 | |
| AI121690_RIKEN_cDNA_1300007C21_gene | −1.4 | −1.9 | −1.2 | −1.1 | |
| AI385457_retinol_binding_protein_2,_cellular | 1.5 | 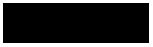 | −1.3 | −1.9 | |
| AA255025_S100_calcium-binding_protein_A9_(calgranulin_B) | −1.1 | −3.10648˜ | 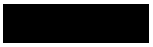 | 1.0 | |
| AI551662_fos-like_antigen_2 | −1.2 | −1.1 | −1.3 | −1.4 | |
| AA414790_leukotriene_A4_hydrolase | −1.3 | −1.6 | −1.2 | −1.7 | |
| AA014727_tumor-suppressing_subchromosomal_transferable_frag- ment_3 | 1.1 | 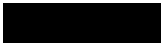 | −1.8 | −3.0 | |
| AA546645_cell_division_cycle_2_homolog_(S._prombe)-like_2 | 1.1 | 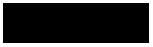 | −1.5 | −3.0 | |
| AA444538_ESTs,_Weakly_similar_to_MDP1_MOUSE_MICRO- SOMAL_DIPEPTIDE_PRECURSOR_[M.musculus] | −1.1 | −1.9886+˜ | 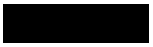 | 1.0 | |
| AA608466_ESTs | −1.2 | 1.17951+˜ | 1.1 | 1.0 | |
| AI386257_calbindin-D9K | −53.5 | −4.1 | 1.0 | −1.2 | |
| AA087193_lipocalin_2 | −1.2 | −1.0 | −1.4 | −1.1 | |
| AA034678_rhotekin | 1.2 | 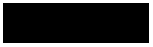 | −1.3 | −2.5 | |
| AA414653_ESTs,_Weakly_similar_to_KIAA0542_protein_[H.sapiens] | −1.2 | 1.28167+˜ | 1.07853+ | 1.07853+ | −1.27379+ |
| AA771229_ESTs,_Weakly_similar_to_zinc_finger_pro- tein_95_[M.musculus] | 1.2 | 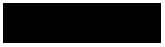 | −2.0 | −3.3 | |
| AA982151_proteinase_3 | 1.1 | 1.44236+˜ | −1.37705+ | −1.4 | |
| AA176045_forkhead_box_C2 | 1.1 | 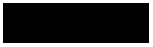 | −1.7 | −3.0 | |
| AA024217_Public_domain_EST | 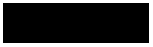 | 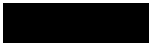 | −1.0 | −4.2 | |
| AI428661_ESTs | 1.2 | 1.2 | −1.6 | −2.6 | |
| AA874101_tumor-associated_calcium_signal_transducer_2 | 1.2 | 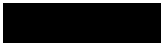 | −1.7 | −2.1 | |
| AA474336_synaptonemal_complex_protein_3 | 1.5 | 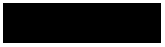 | 1.1 | −3.7 | |
| AA031015_myloperoxidase | 1.5 | 1.05319˜ | −1.3 | 1.0 | |
| AA189266_ESTs | 1.3 | 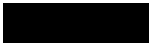 | −1.3 | −5.6 | |
| AA060282_neutrophilic_granule_protein | 1.26109+ | −1.34714+03 ˜ | 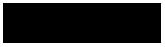 | 1.5 | |

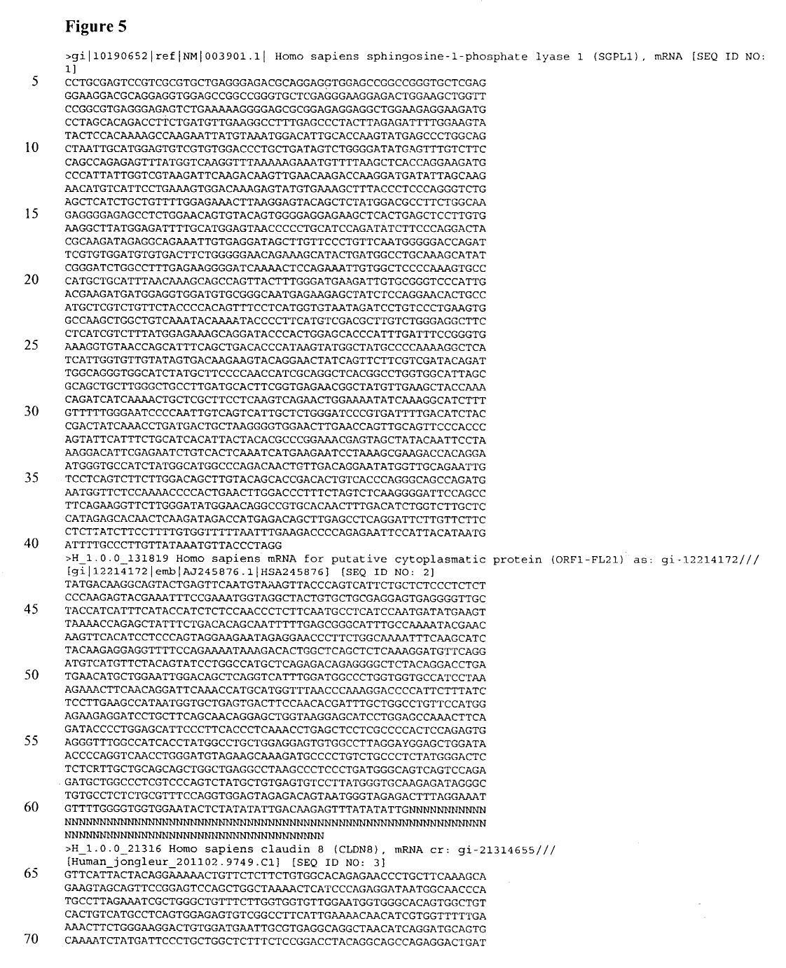

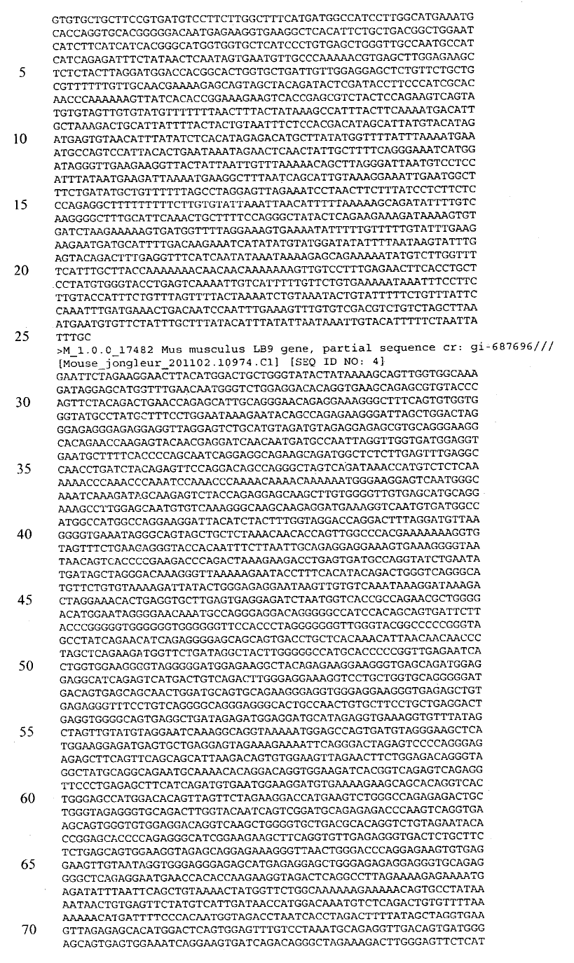

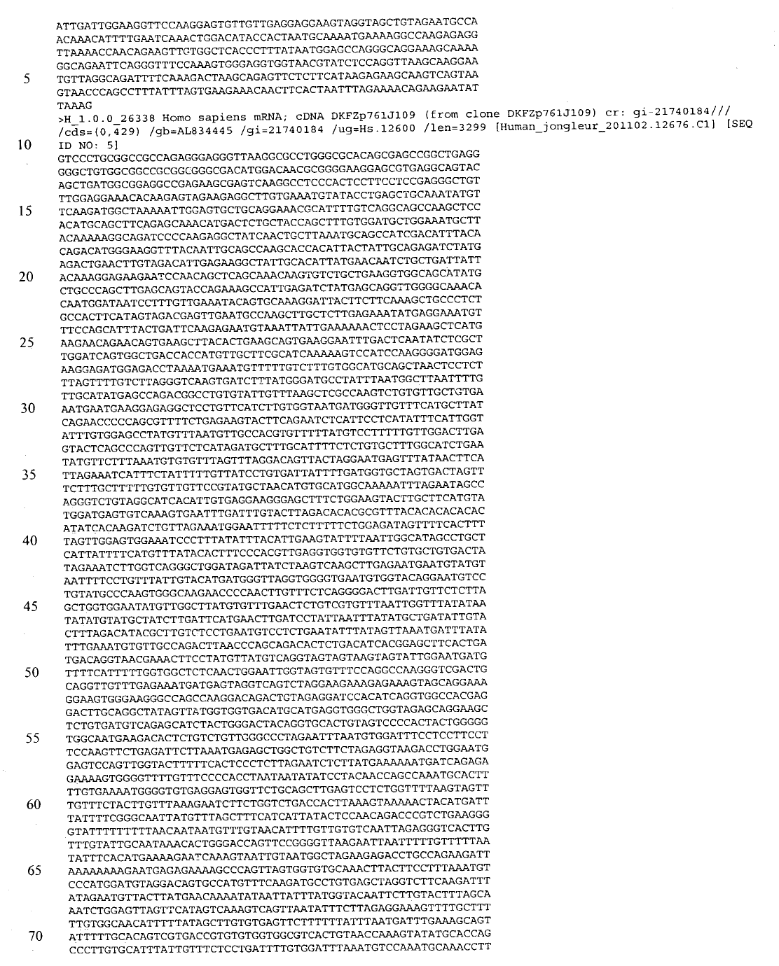

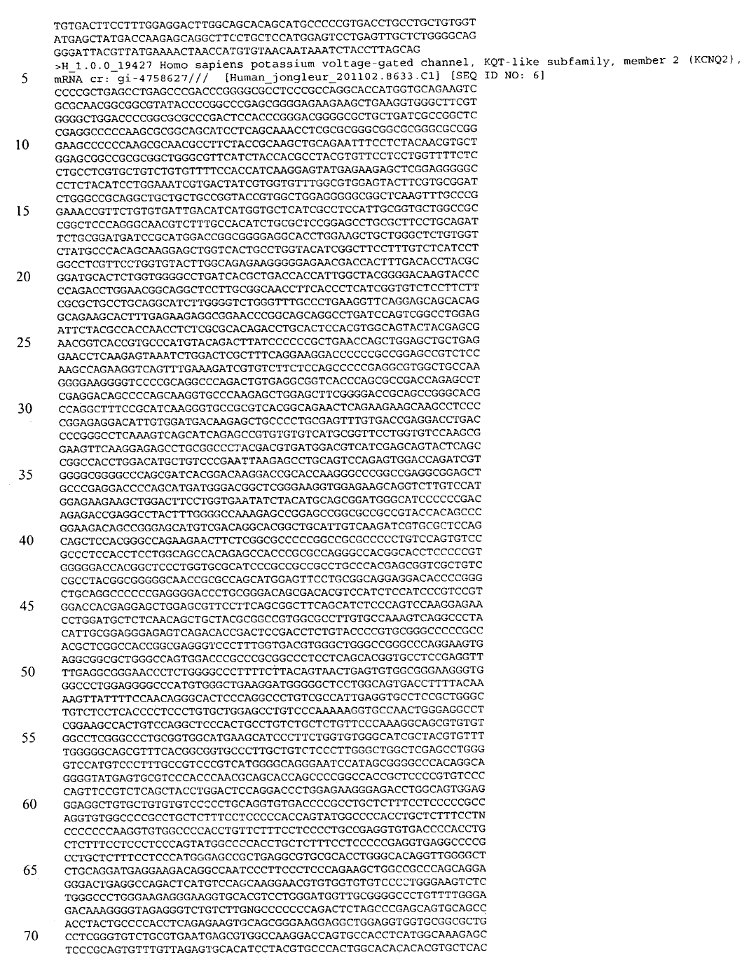

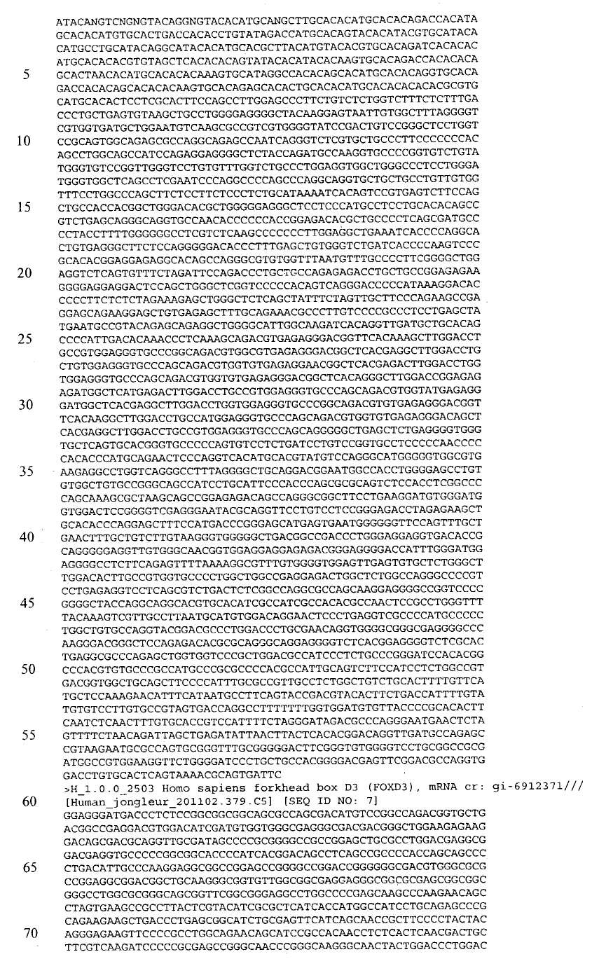

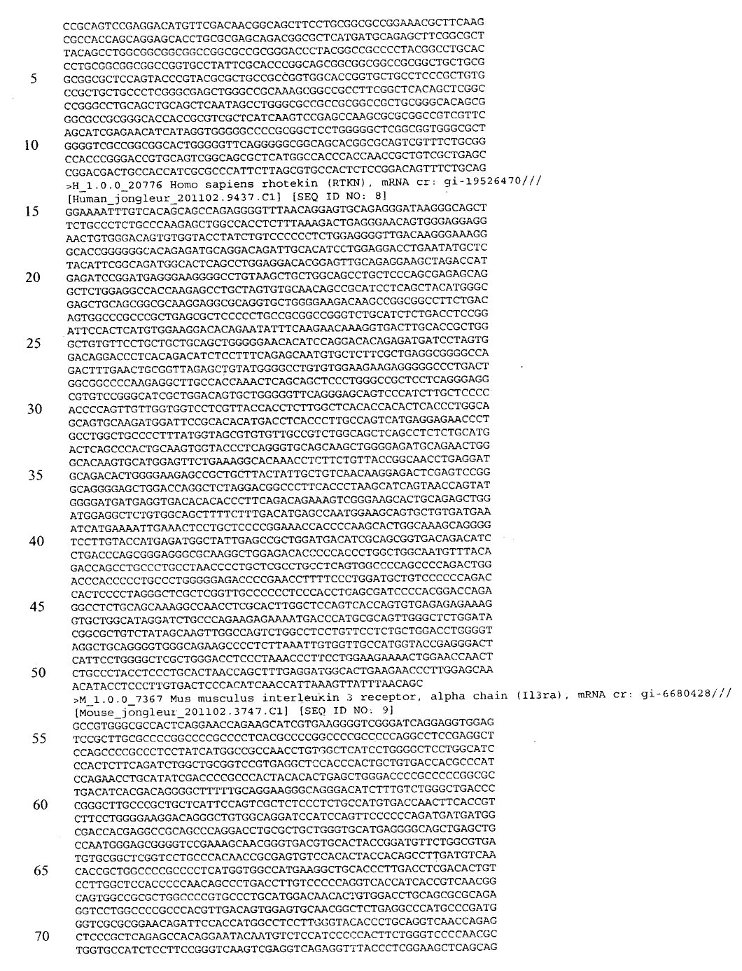

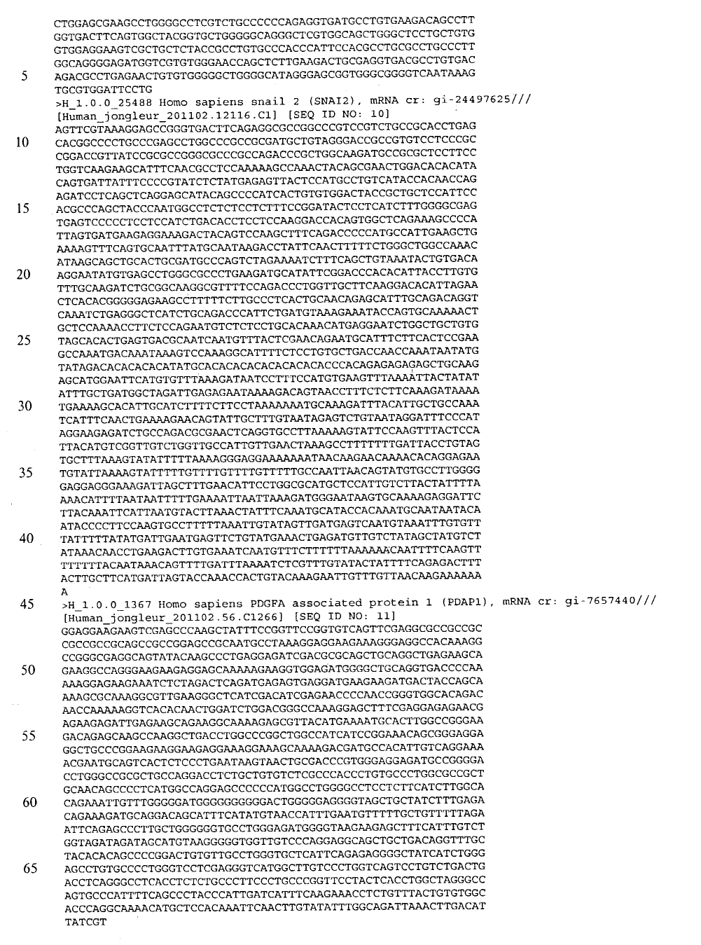

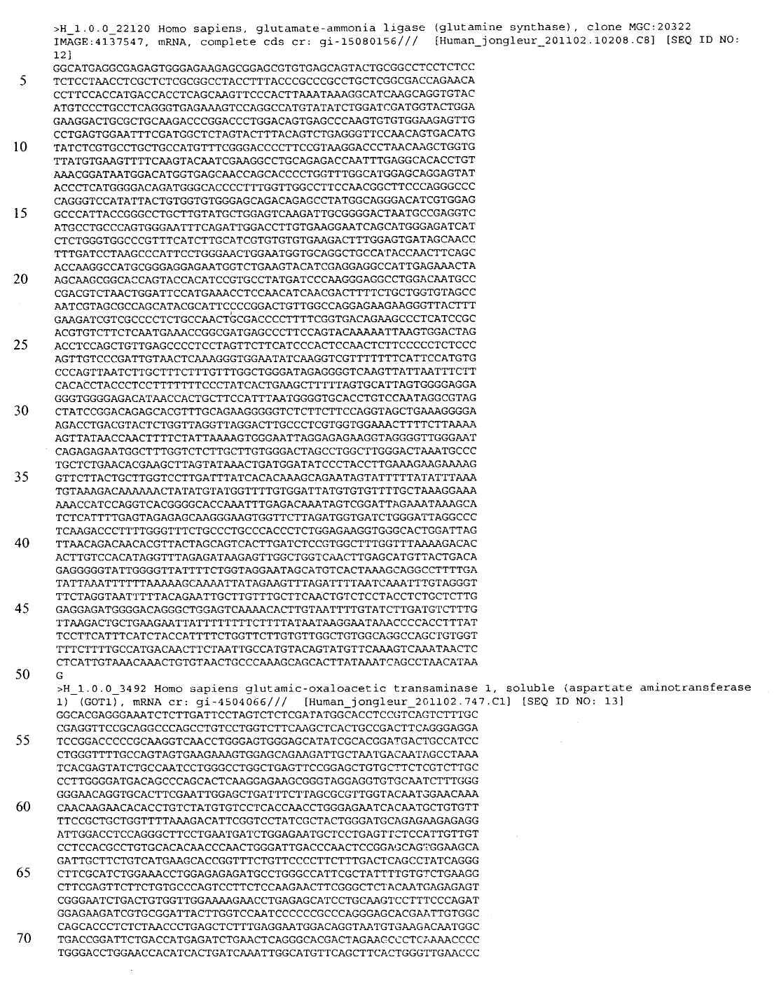

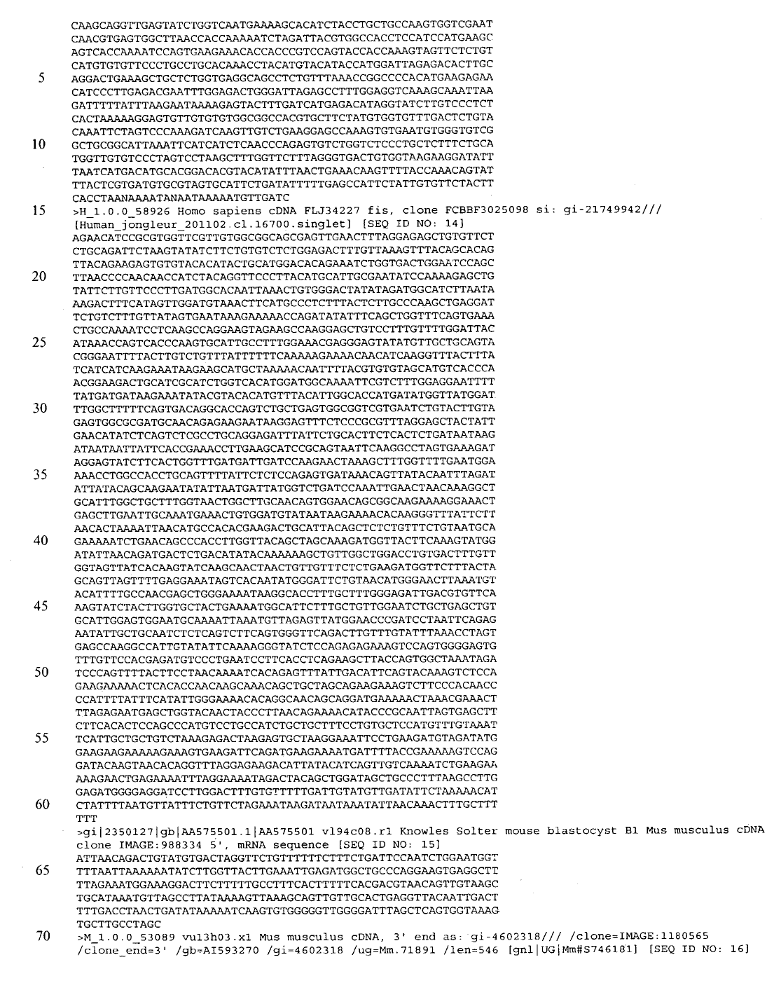

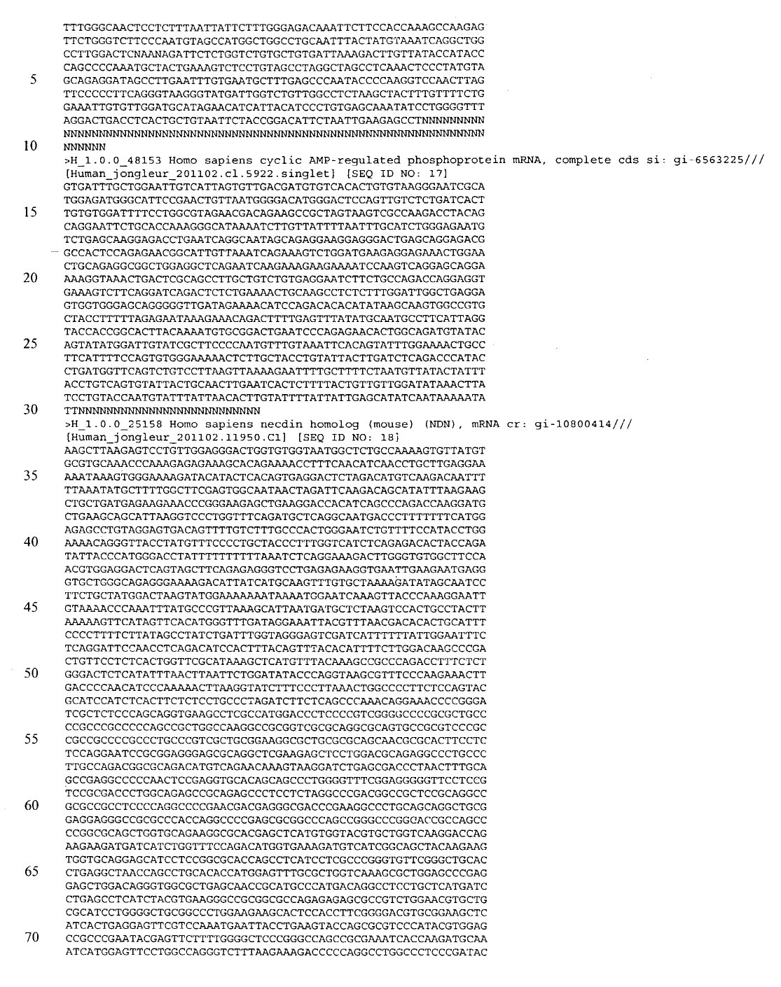

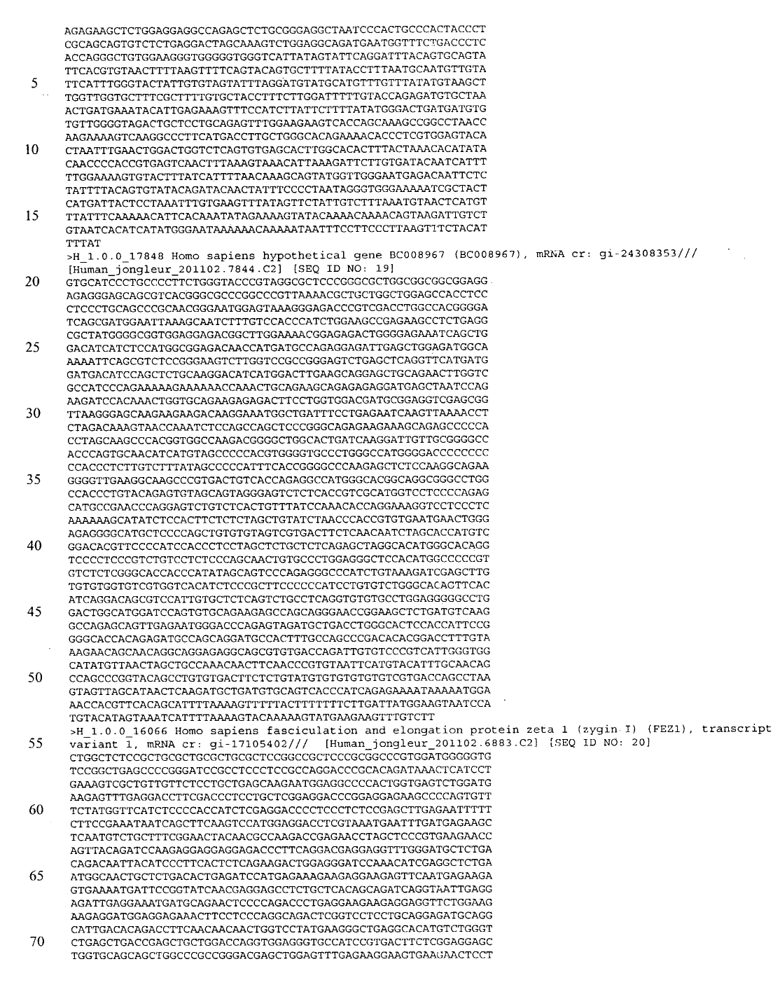

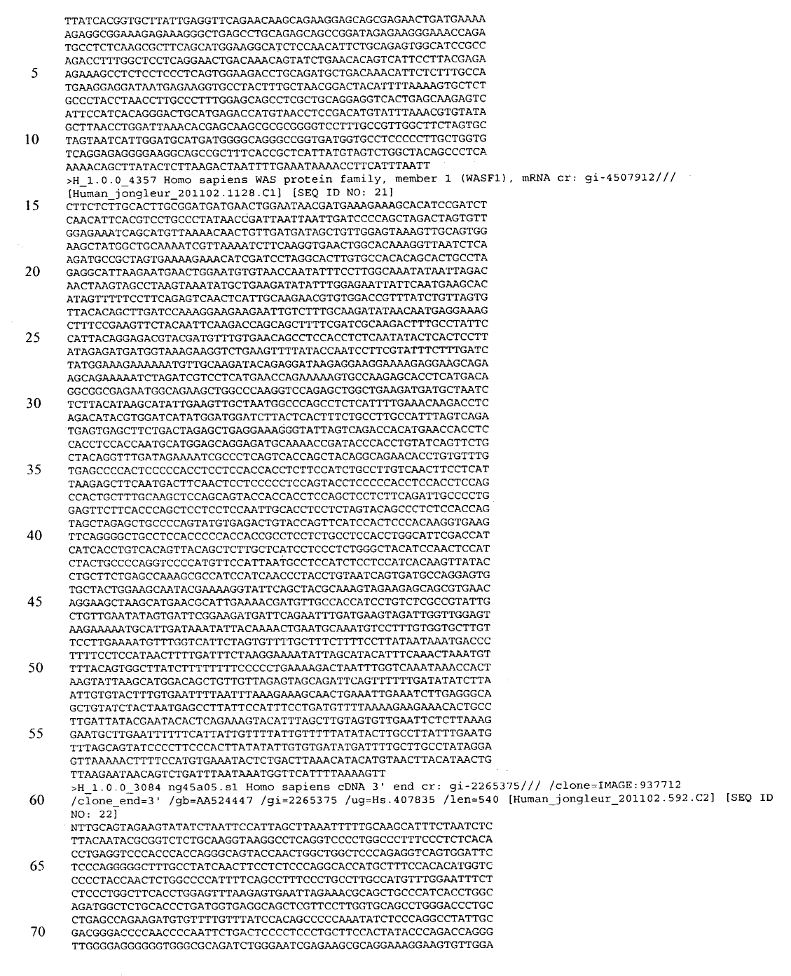

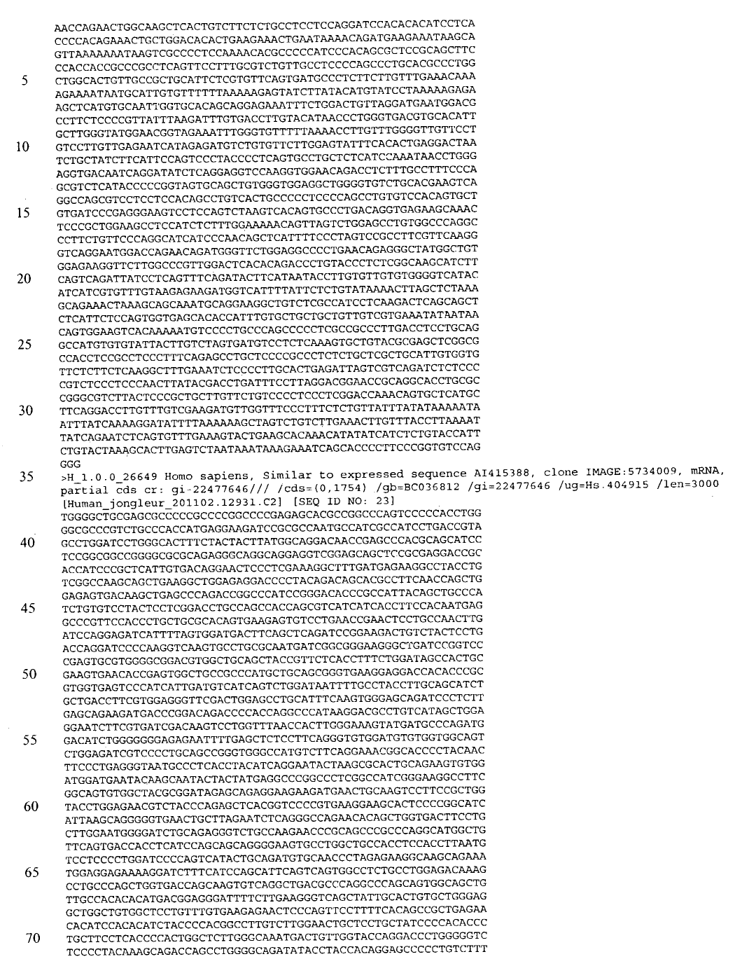

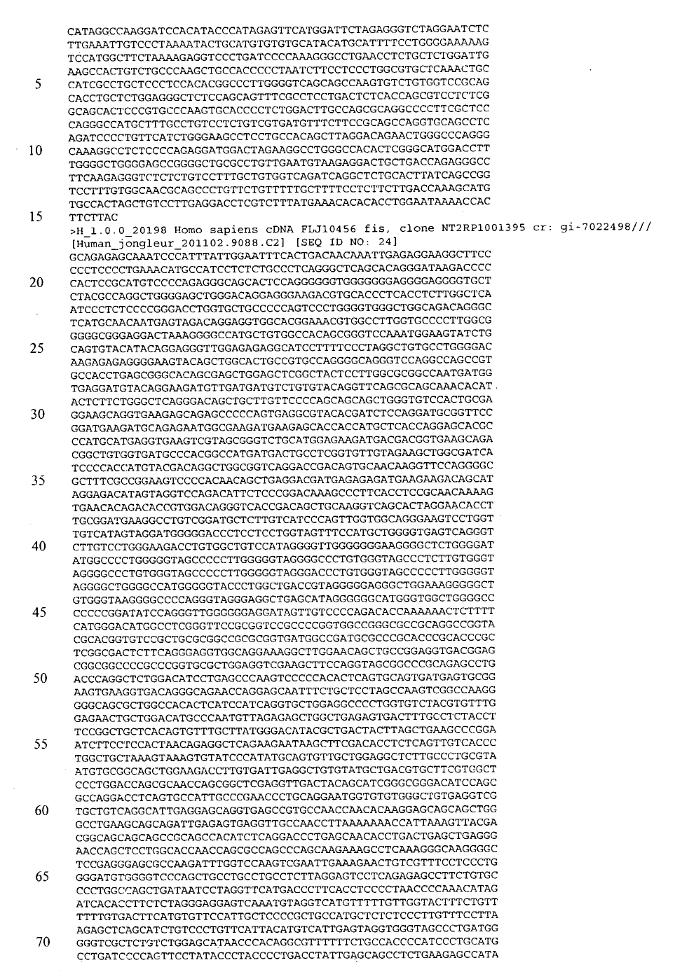

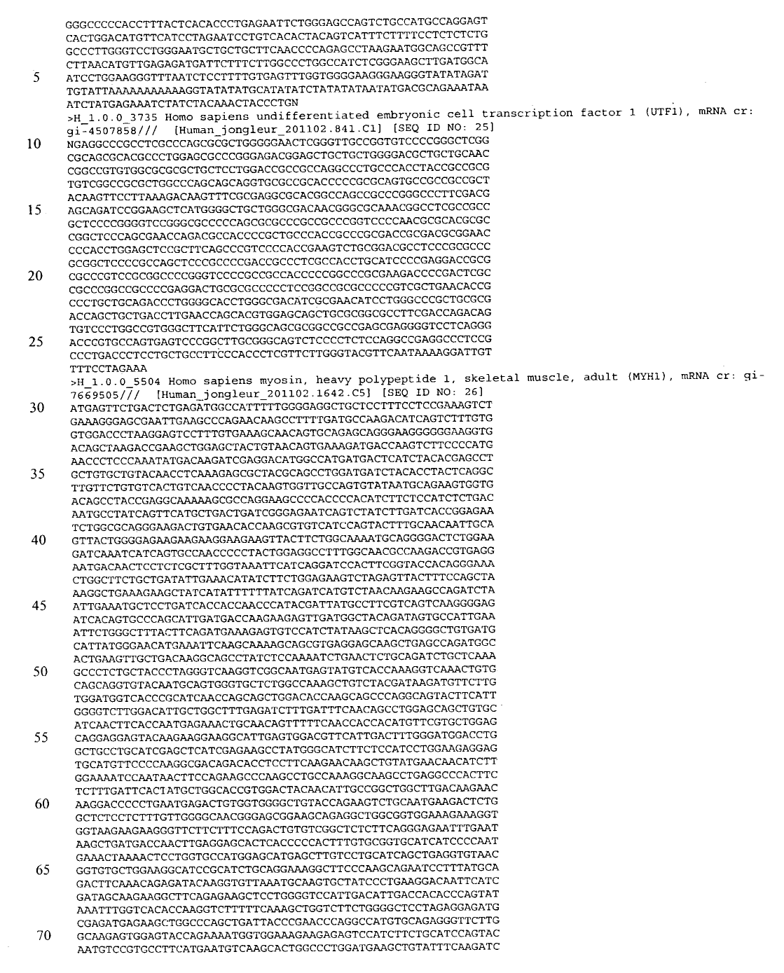

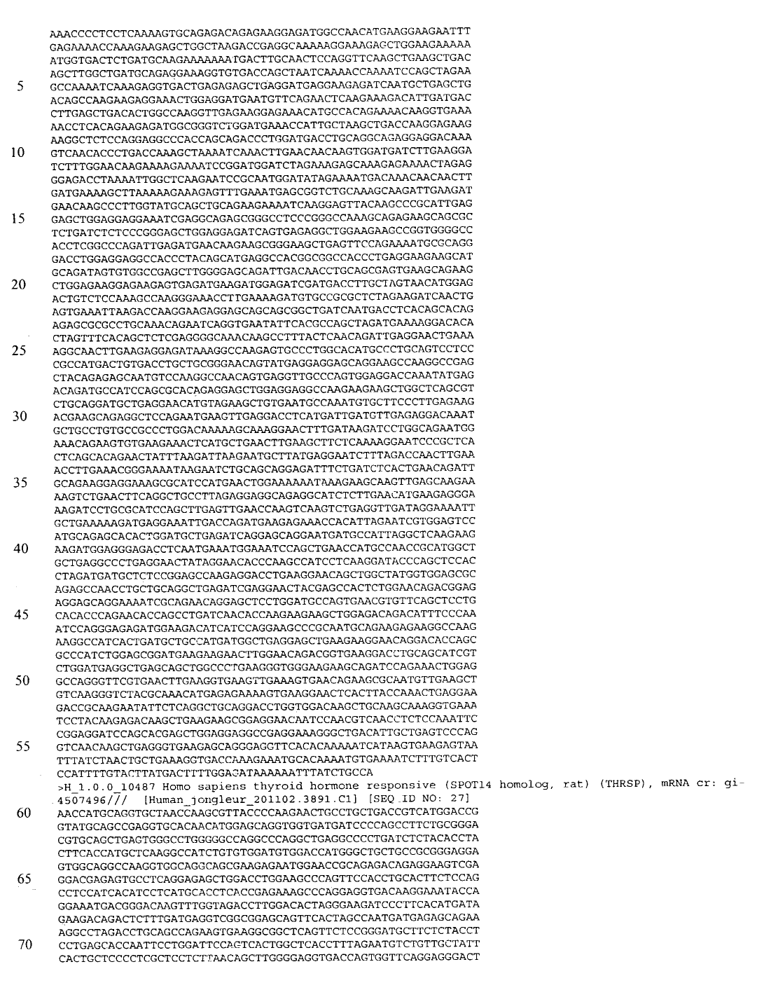

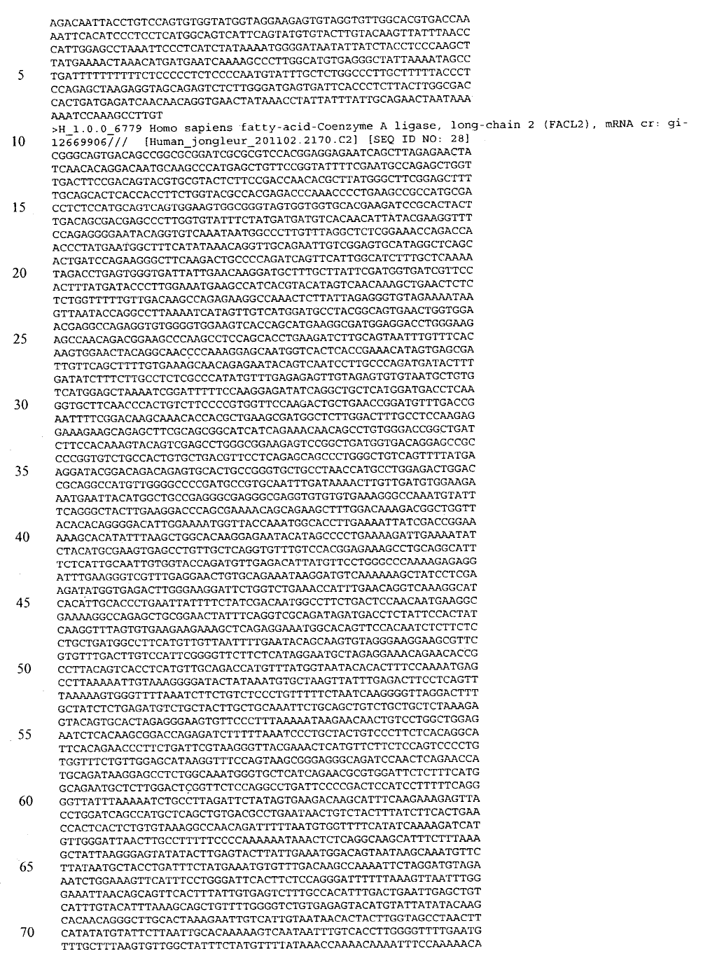

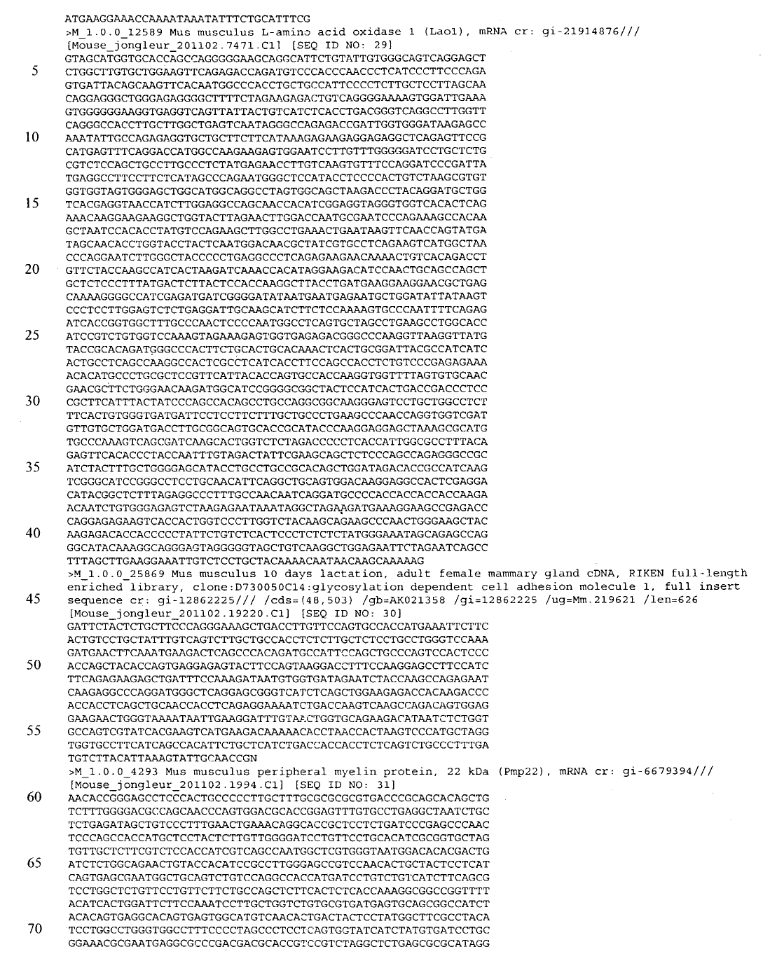

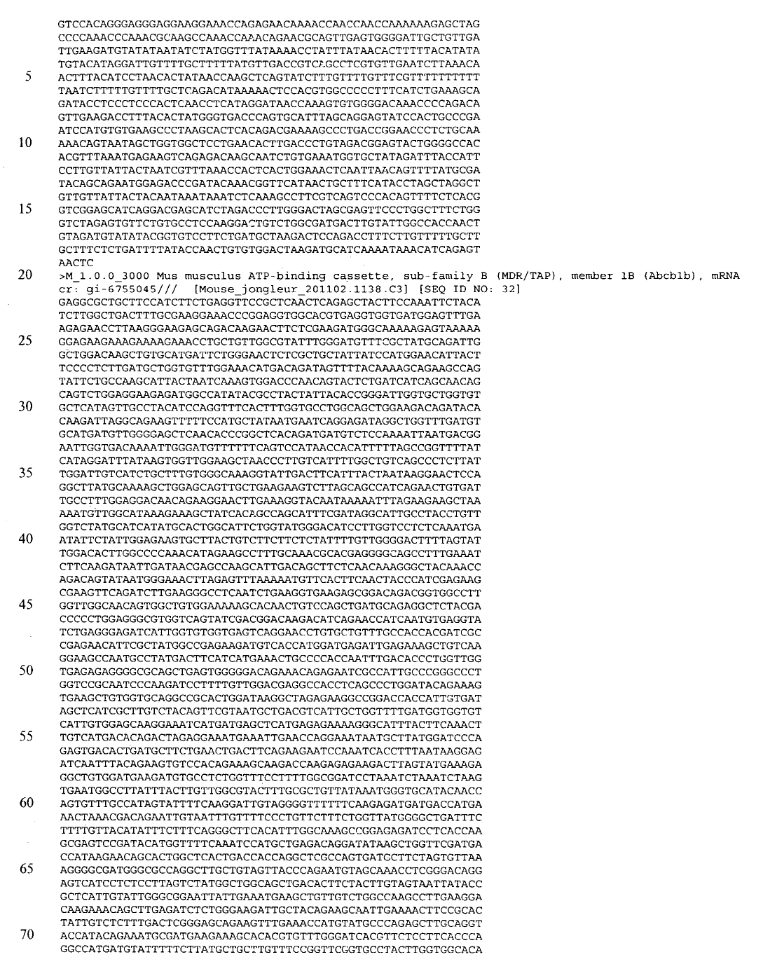

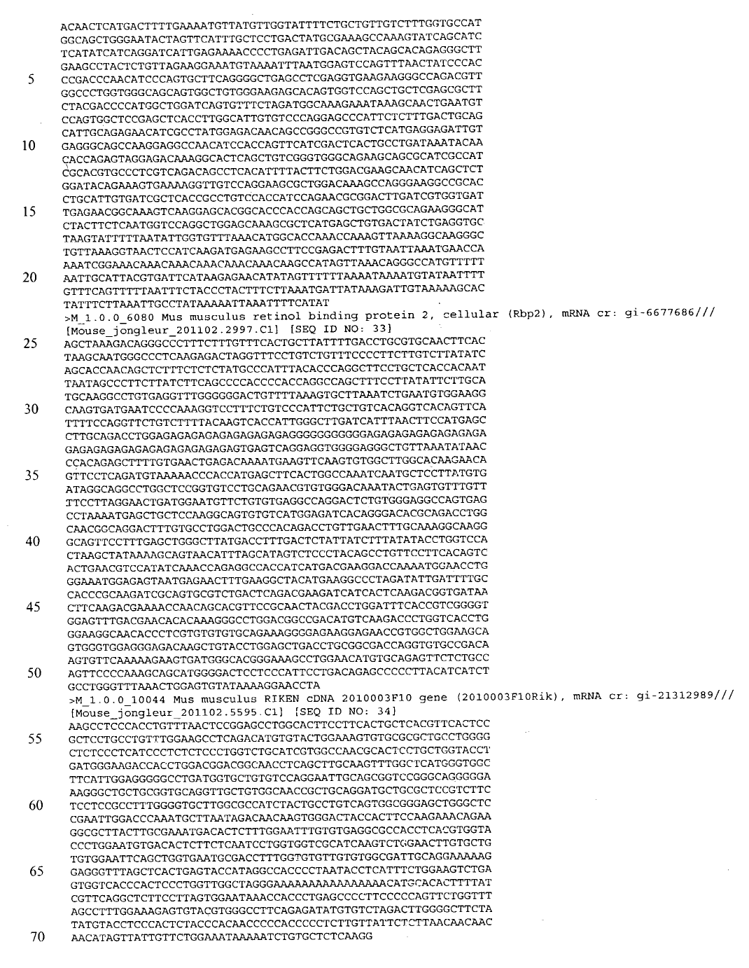

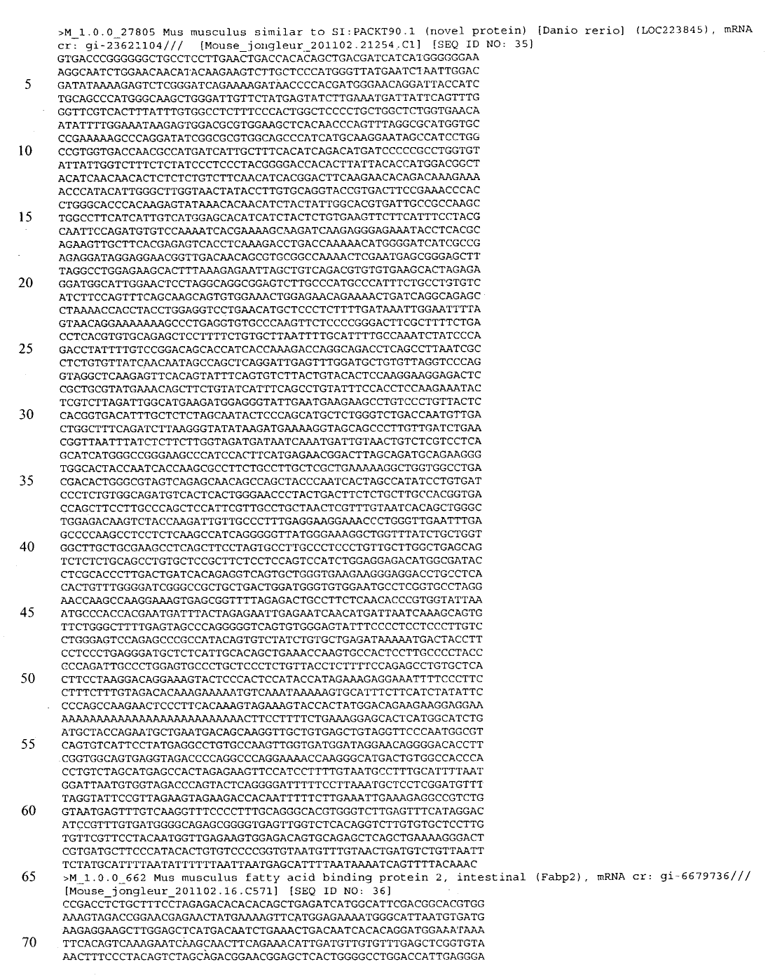

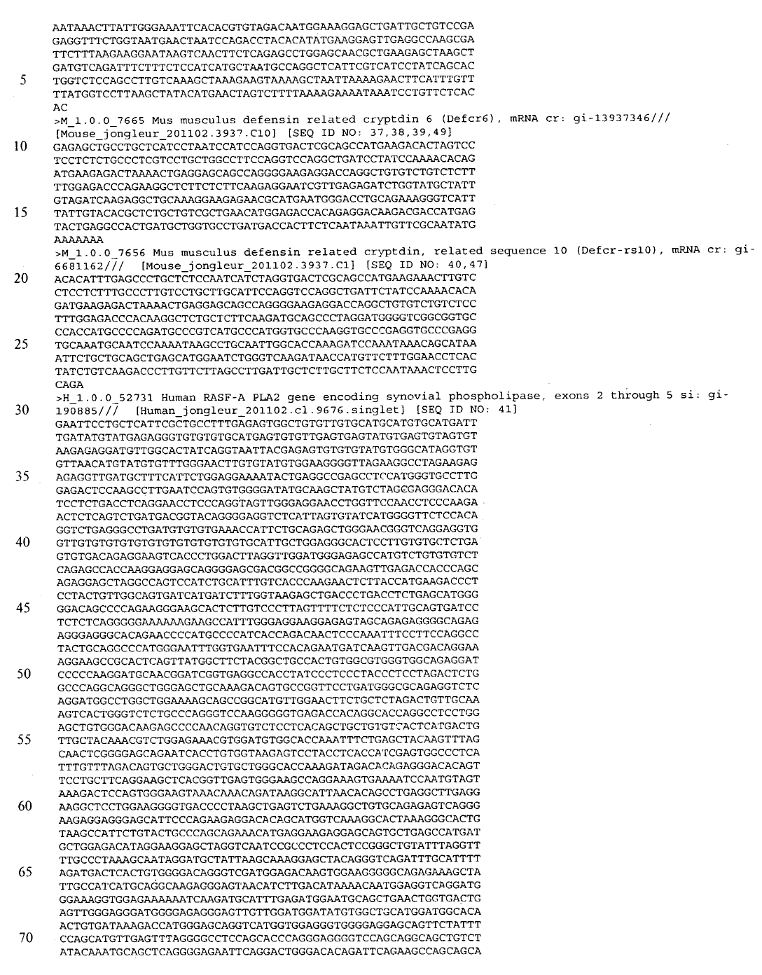

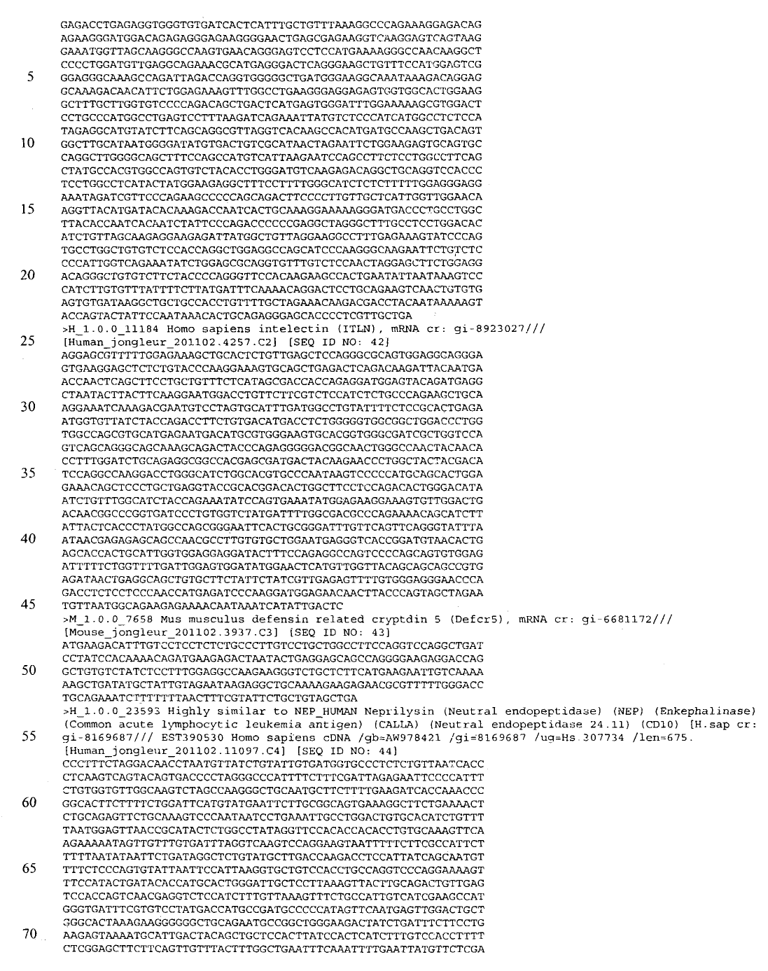

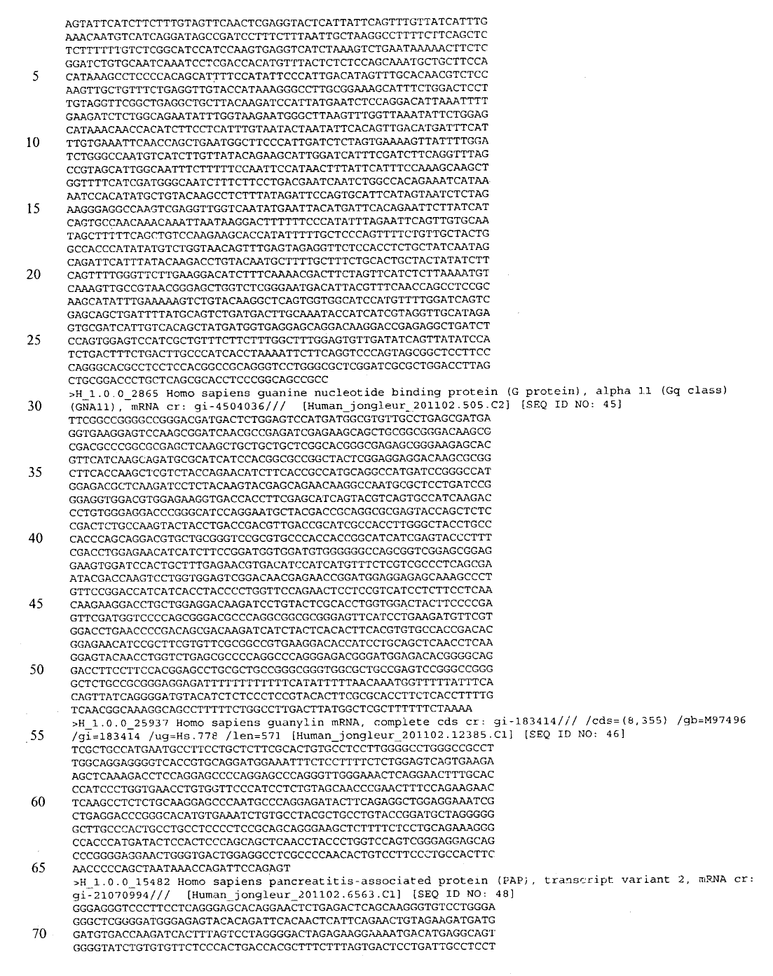

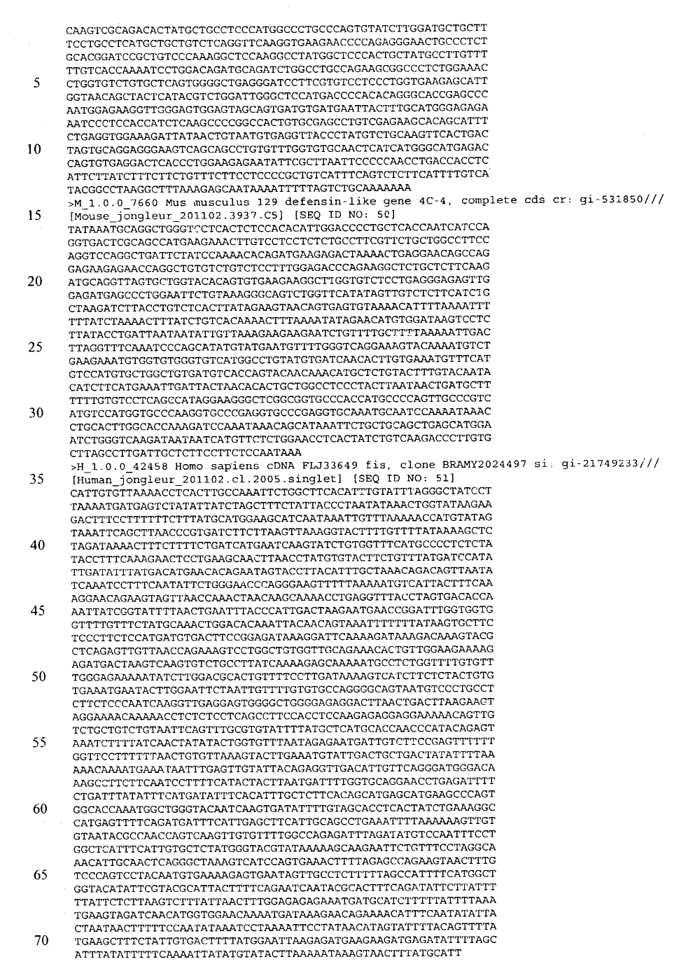

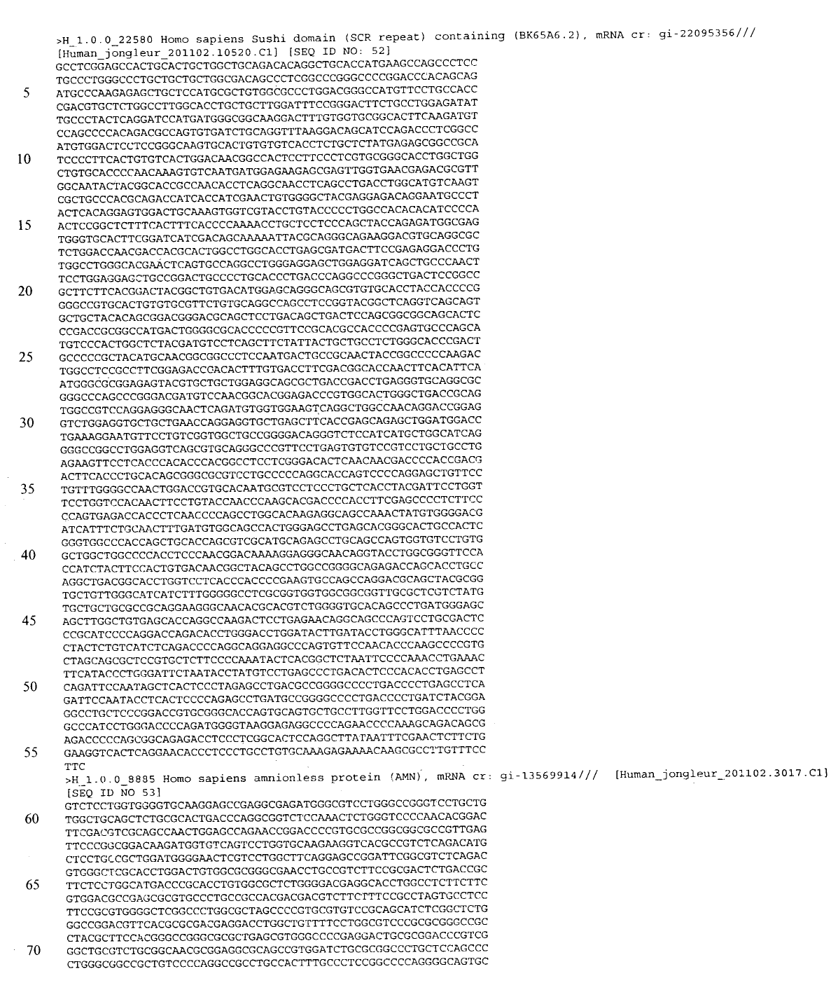

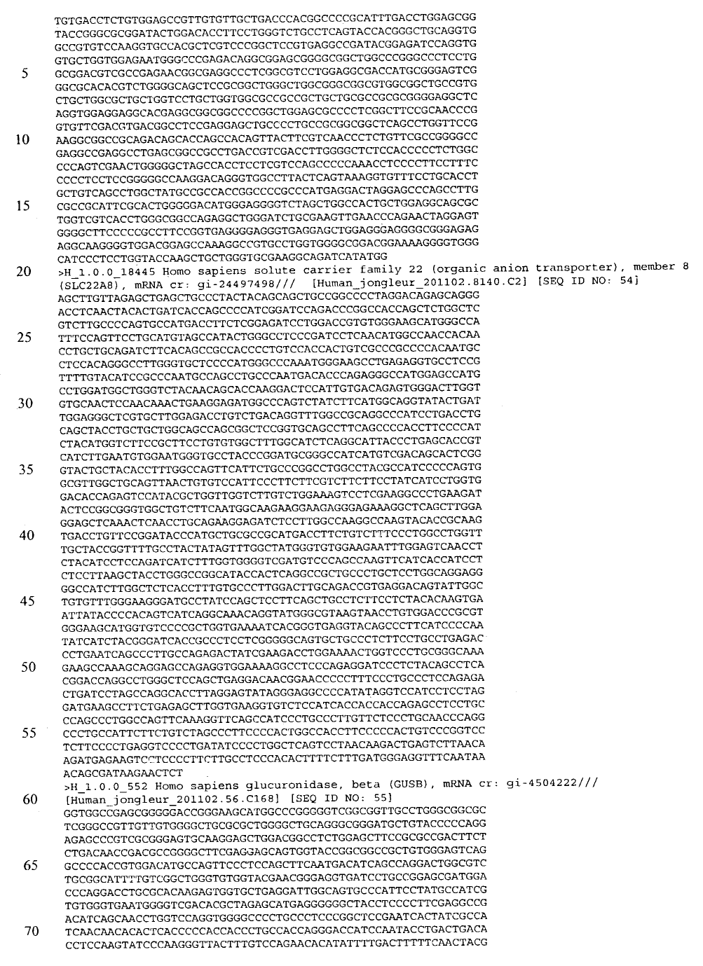

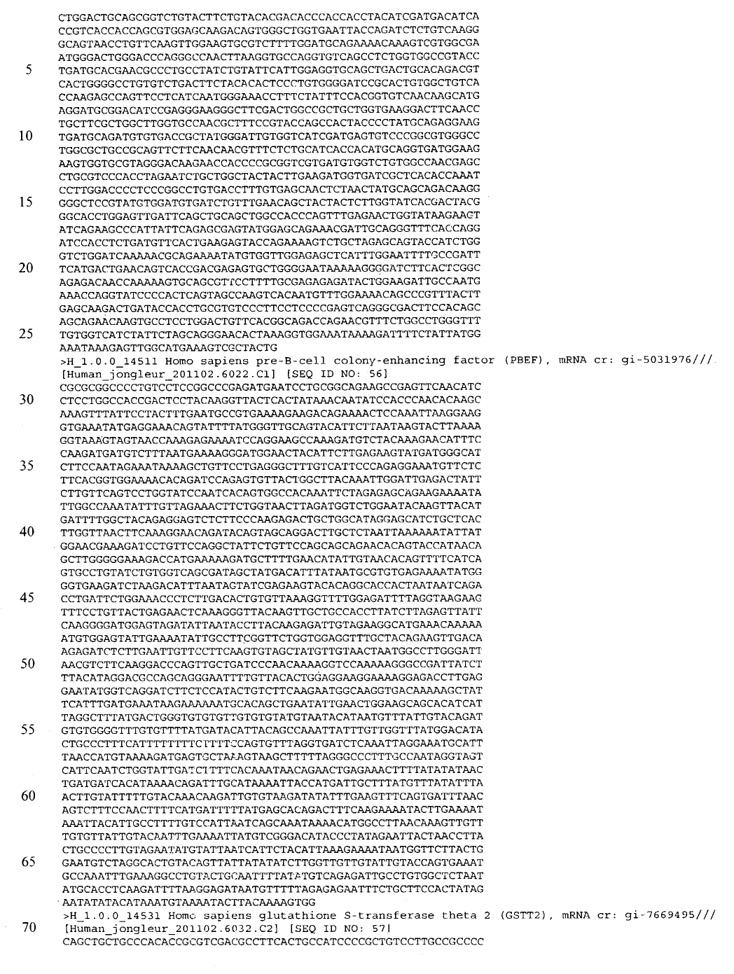

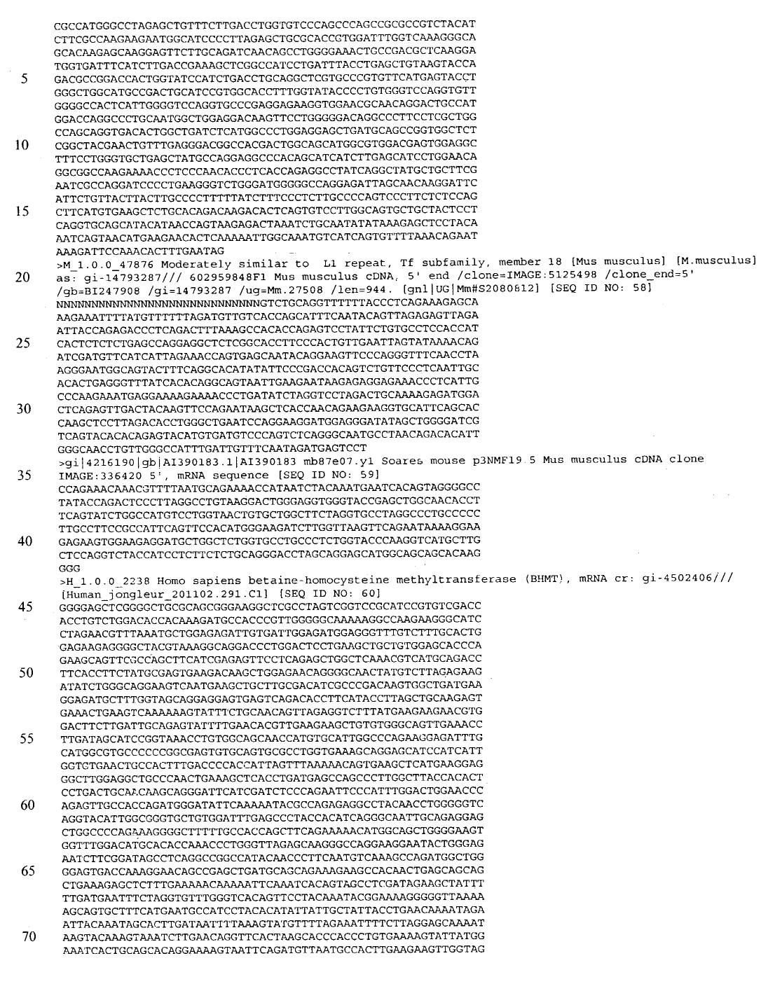

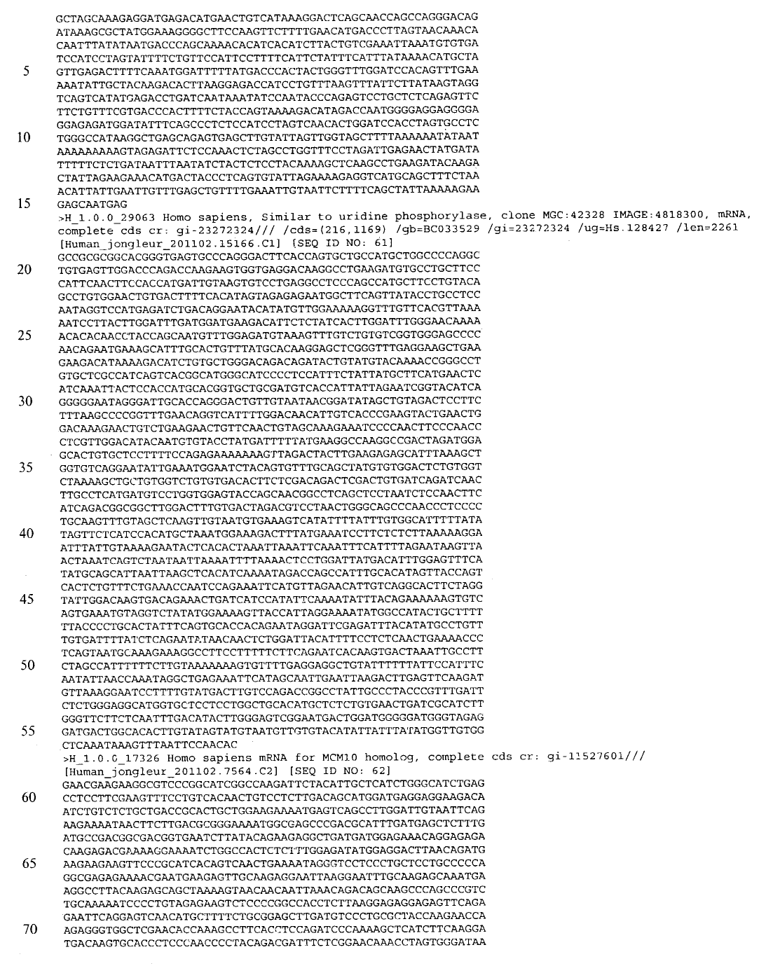

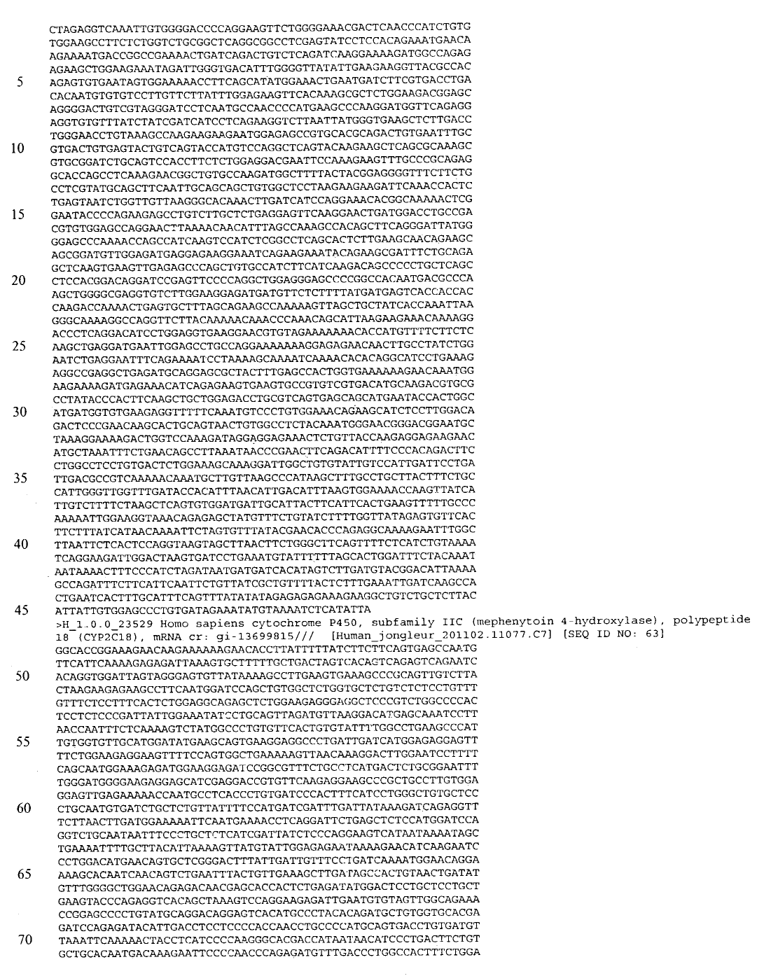

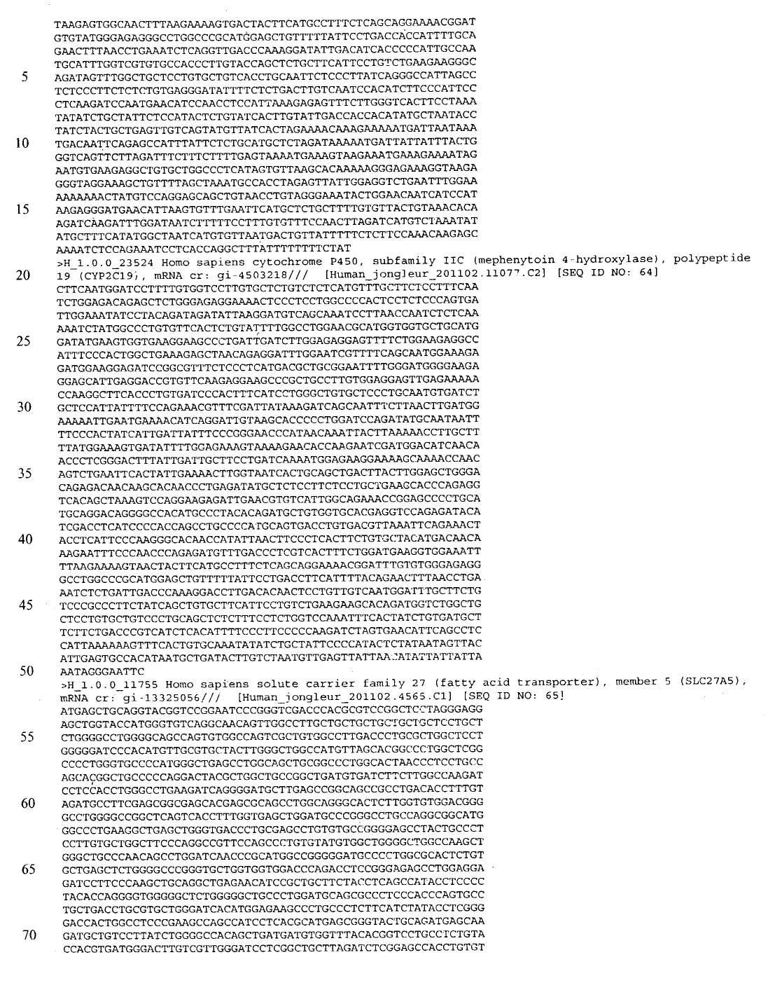

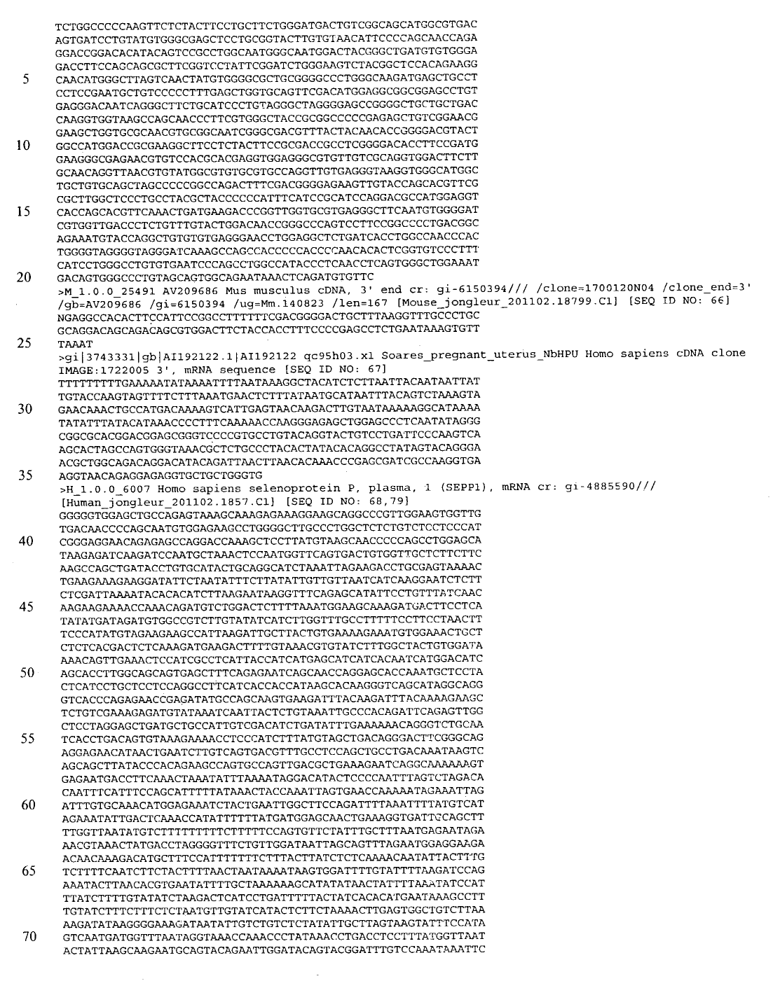

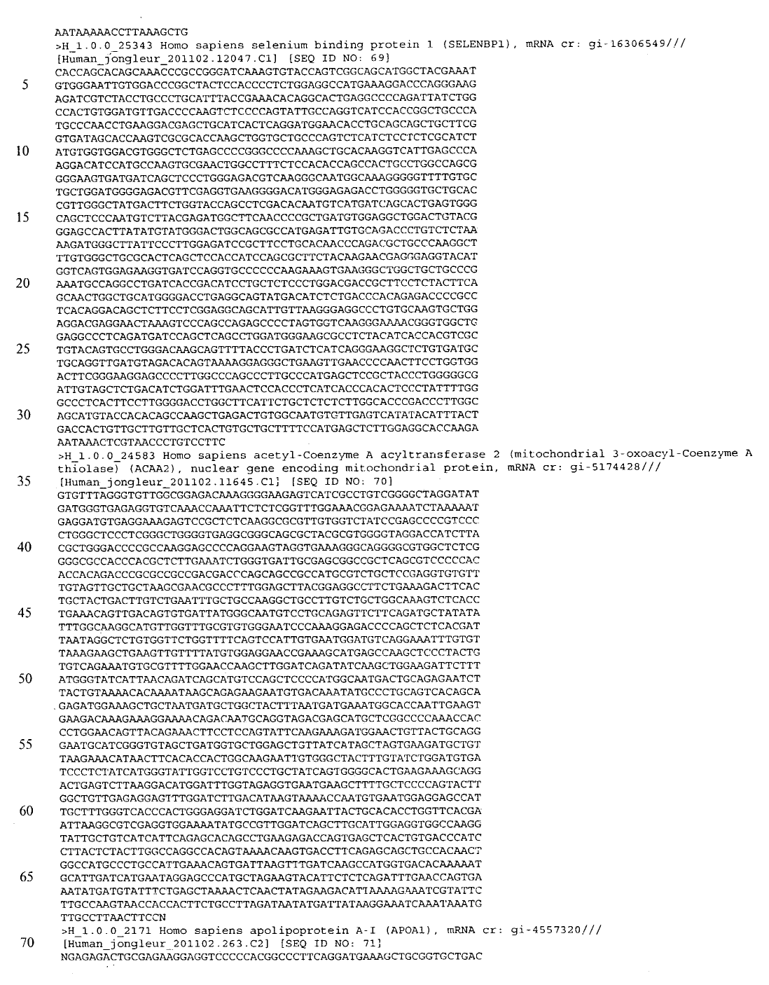

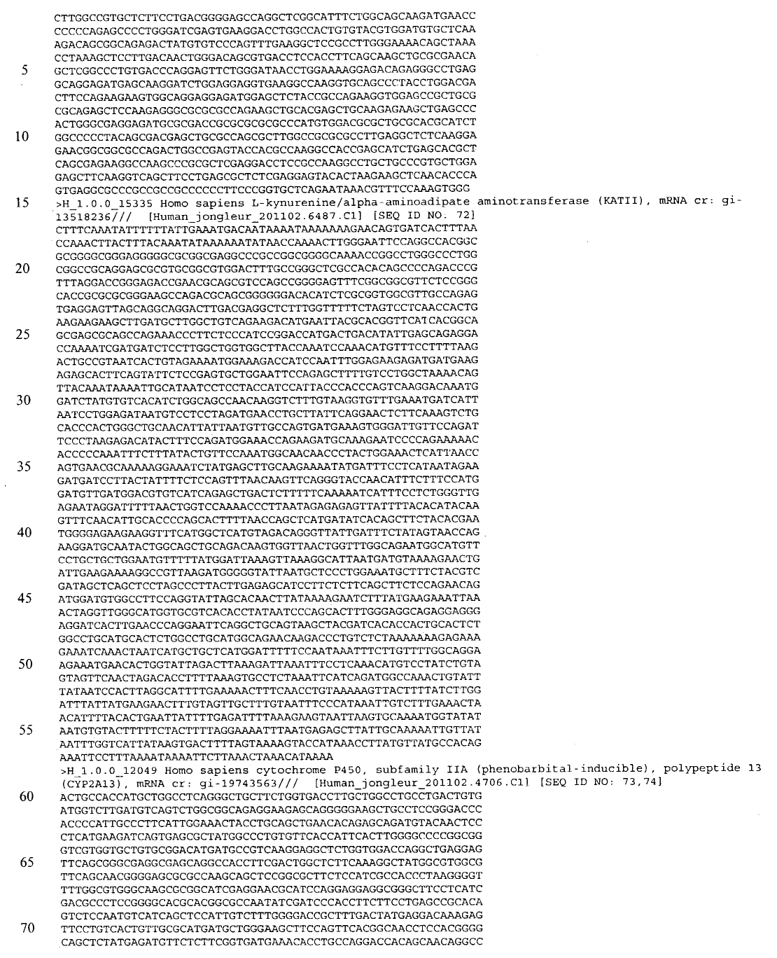

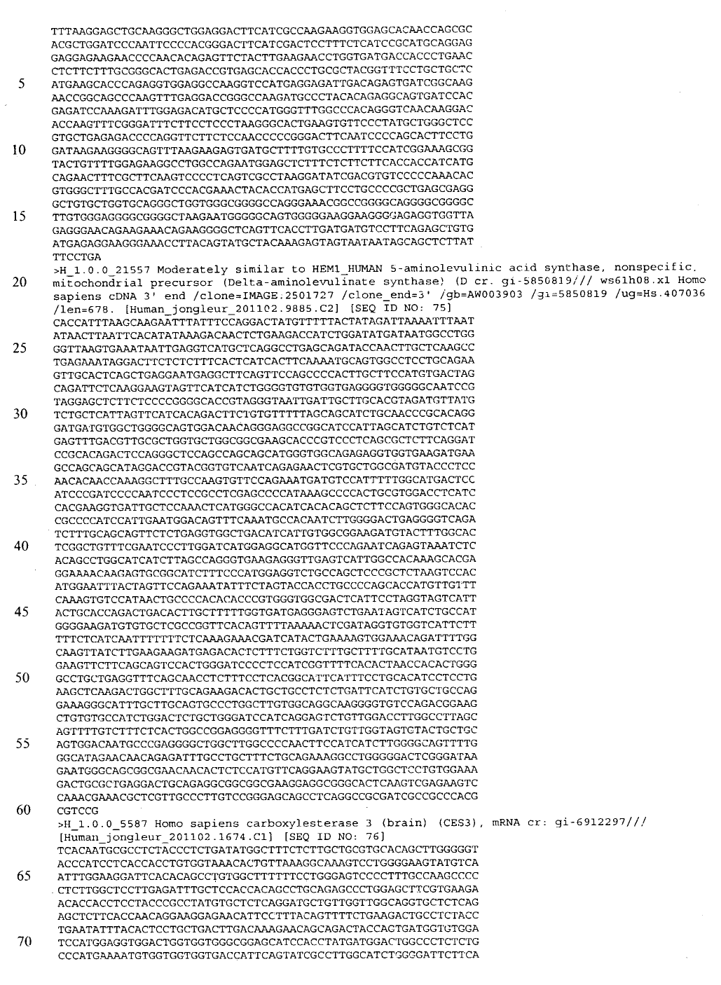

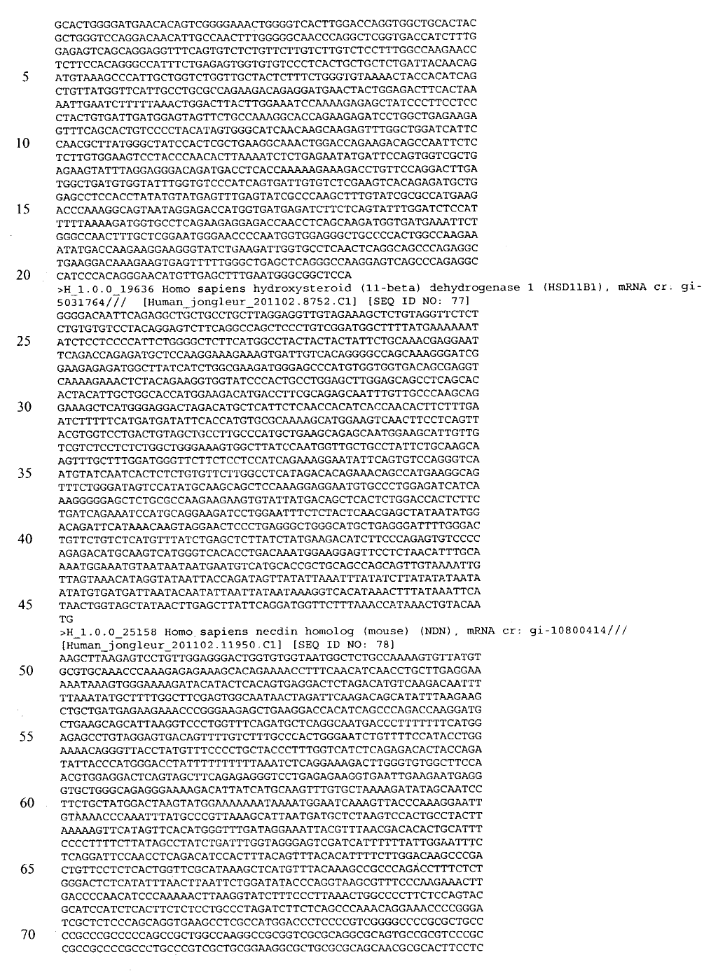

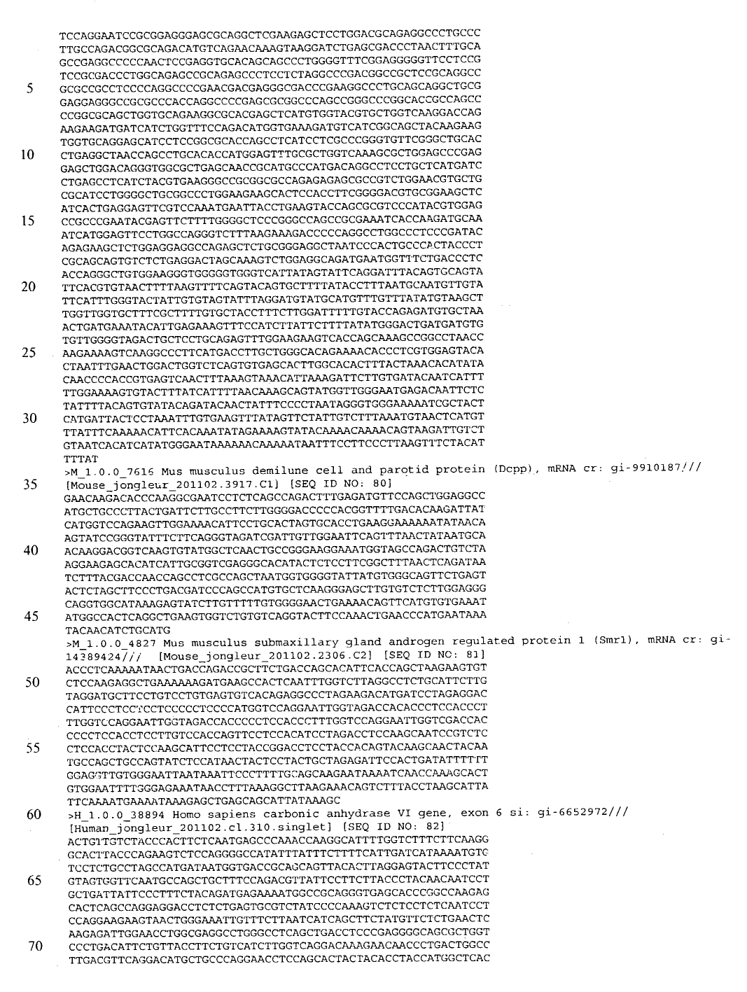

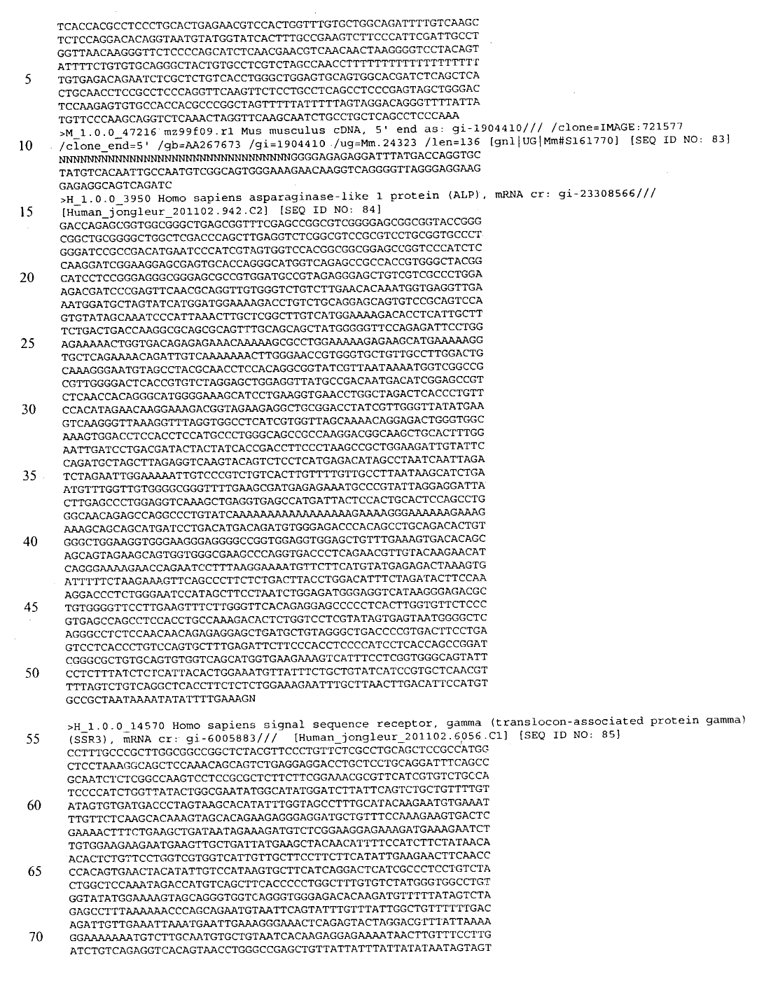

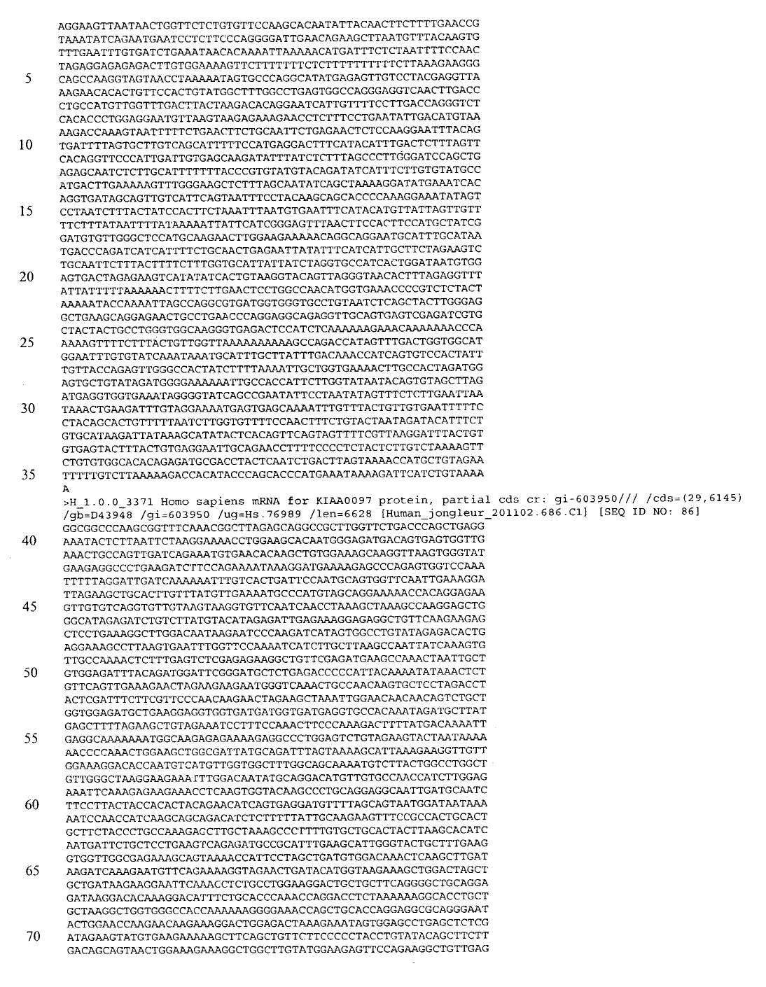

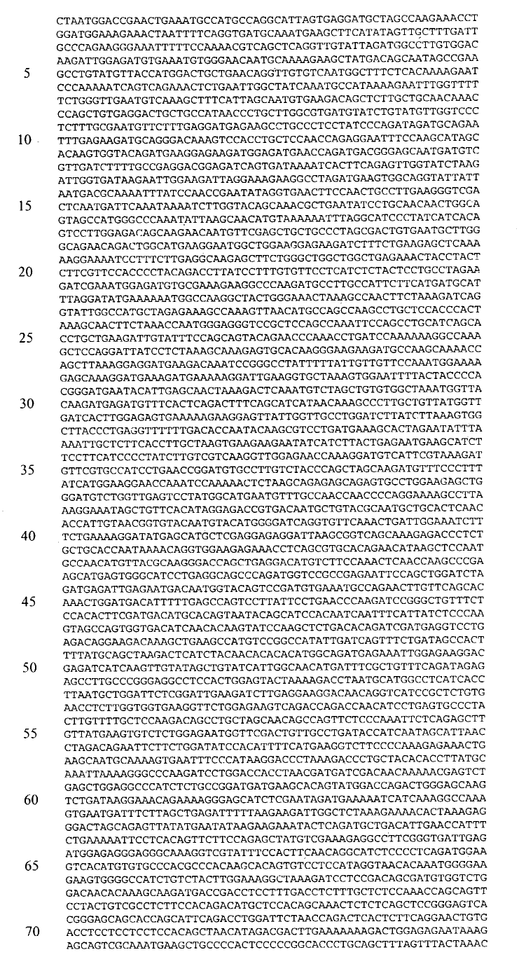

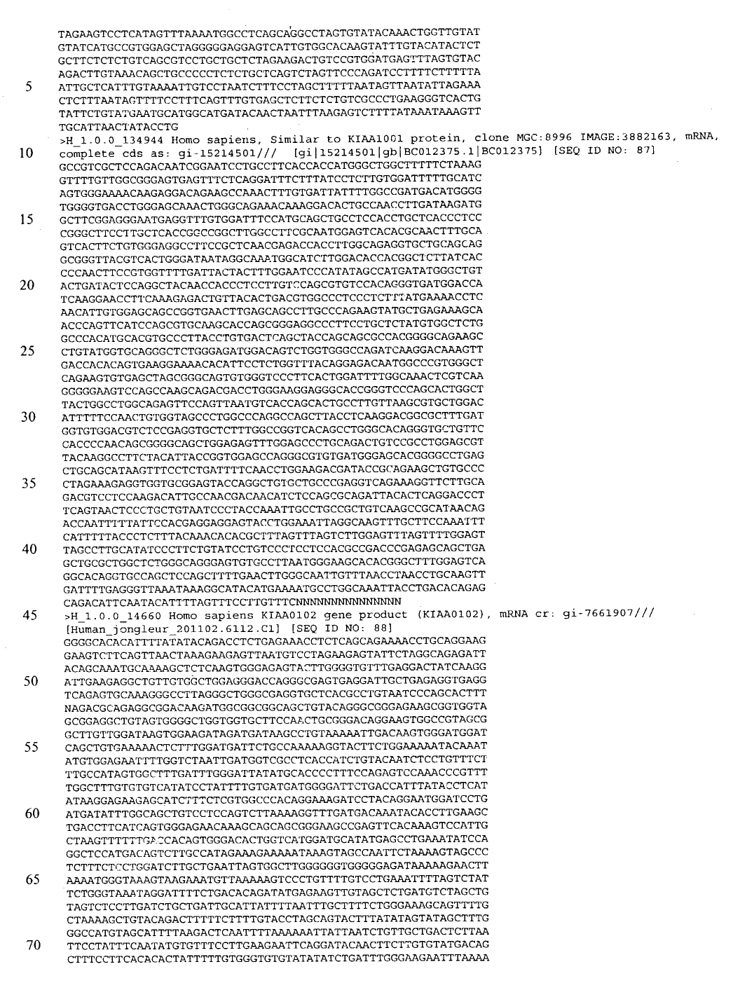

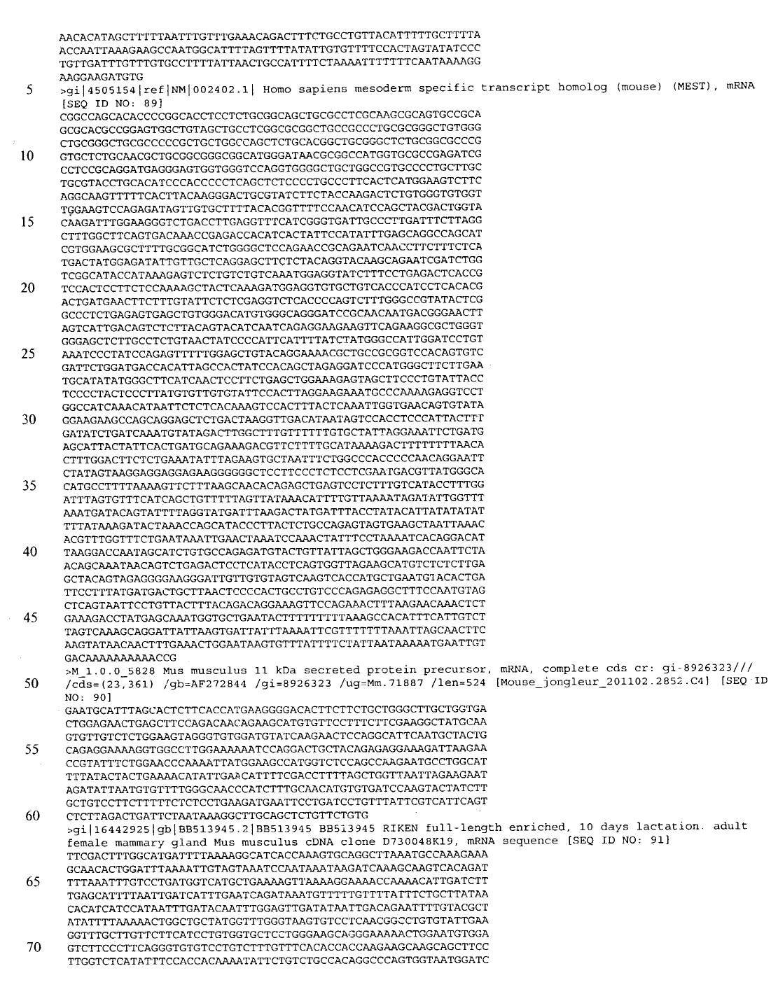

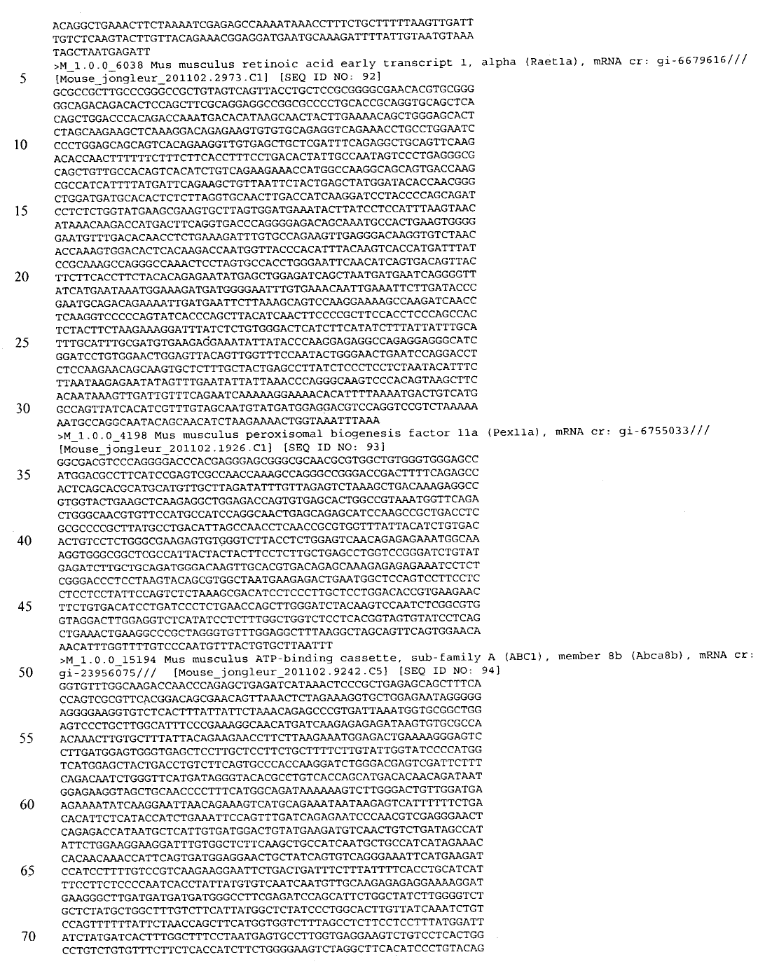

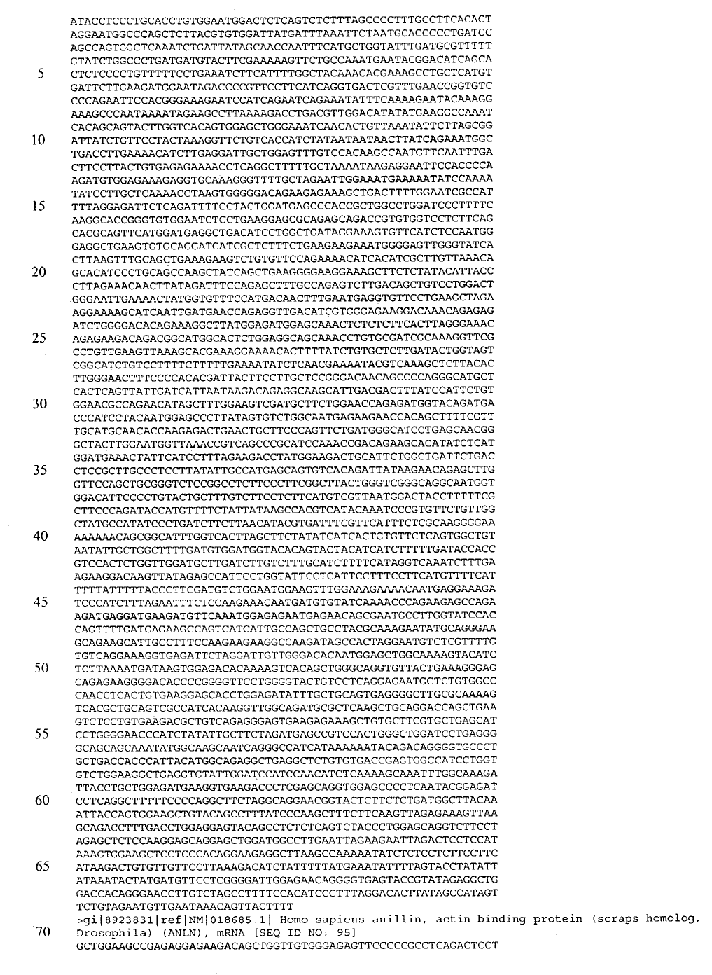

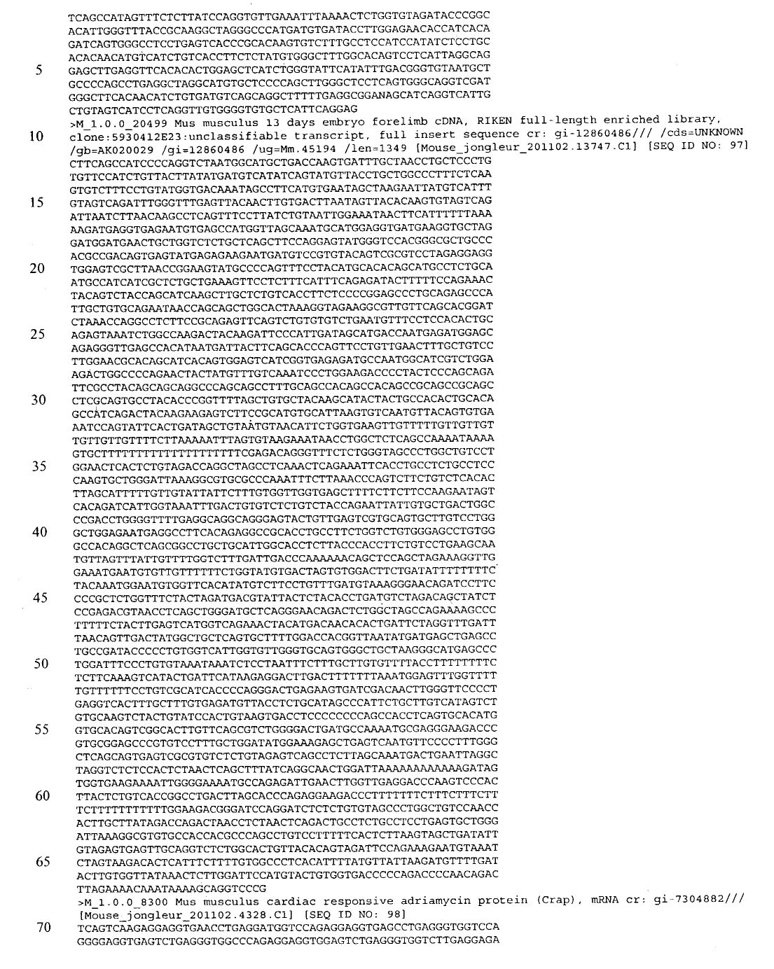

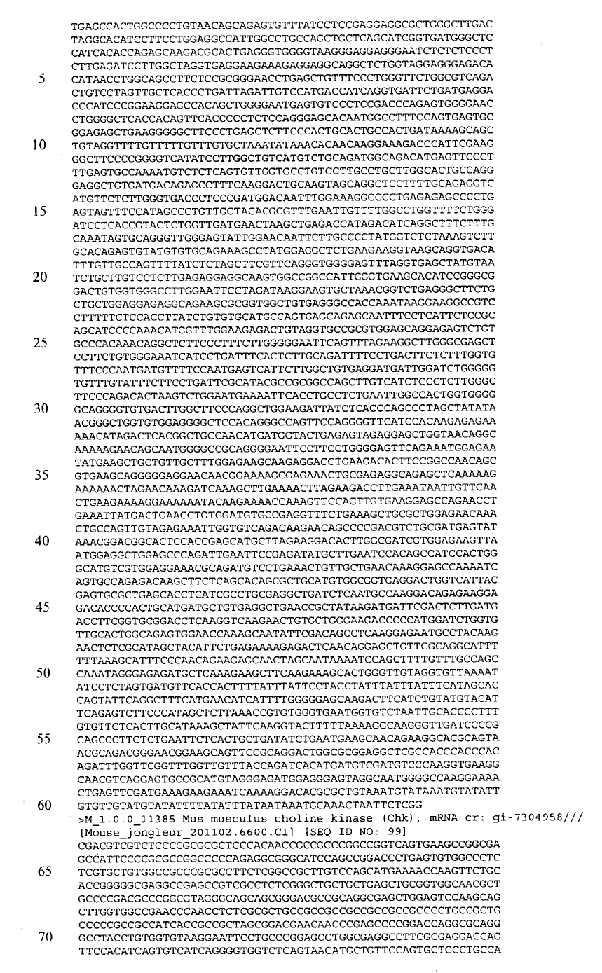

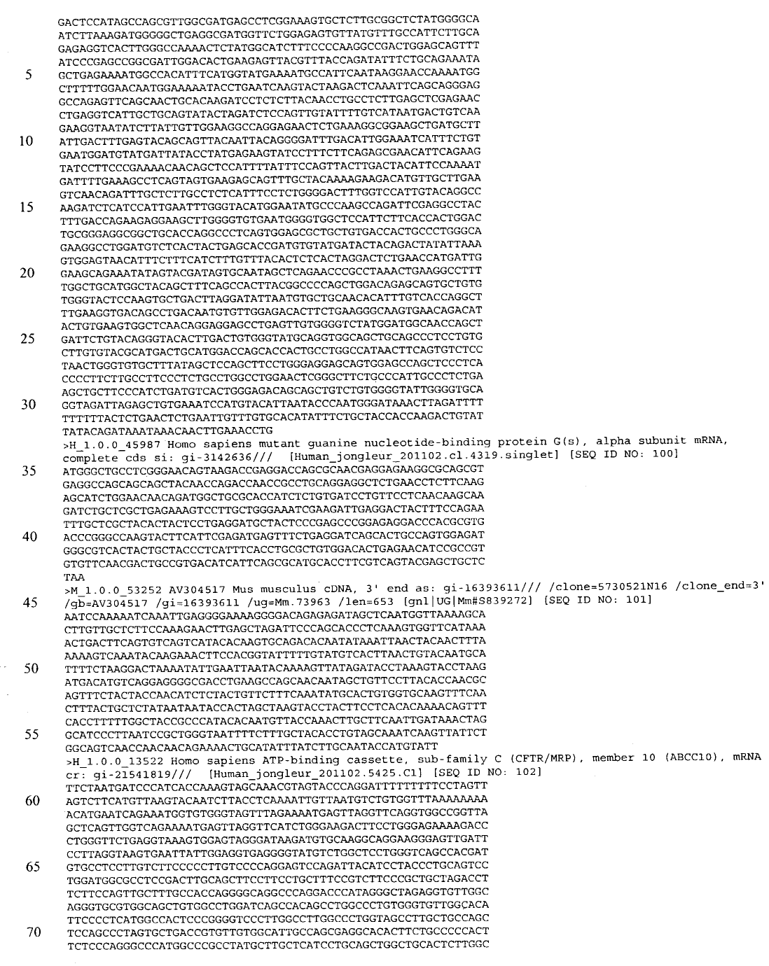

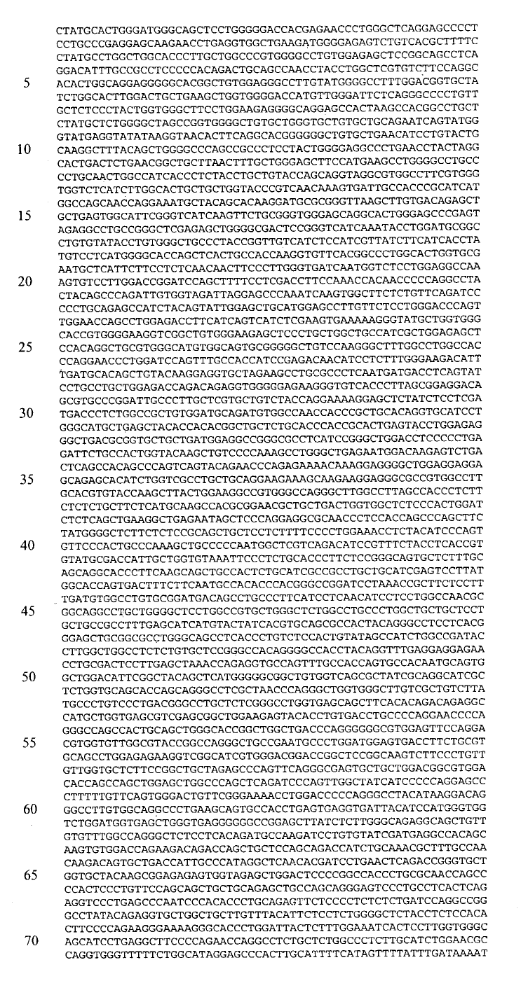

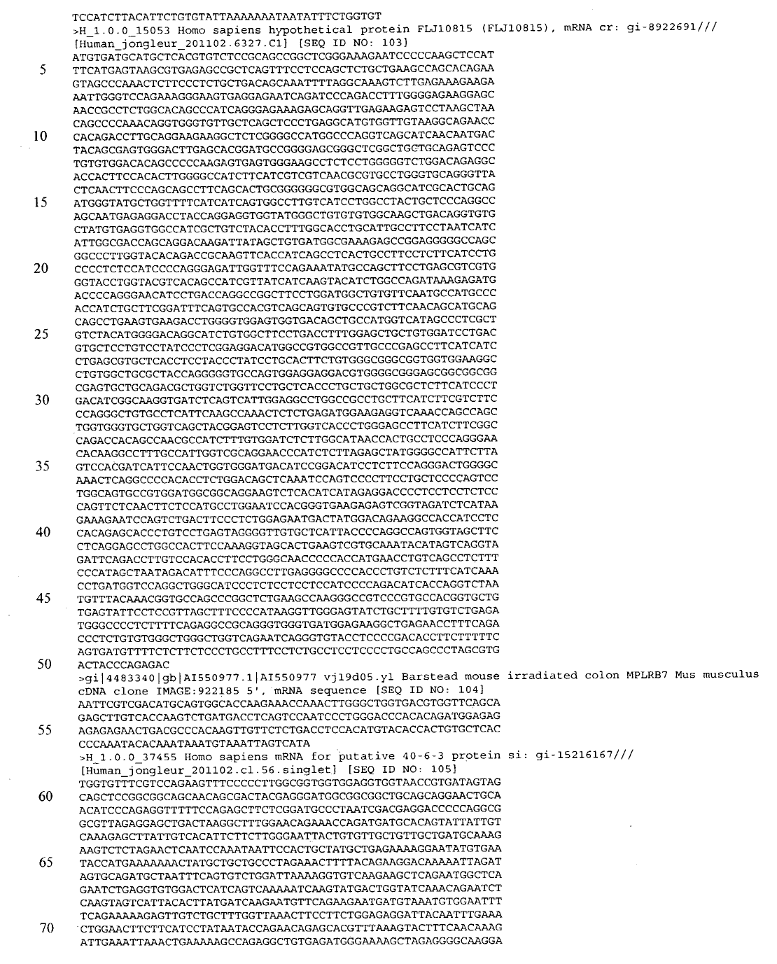

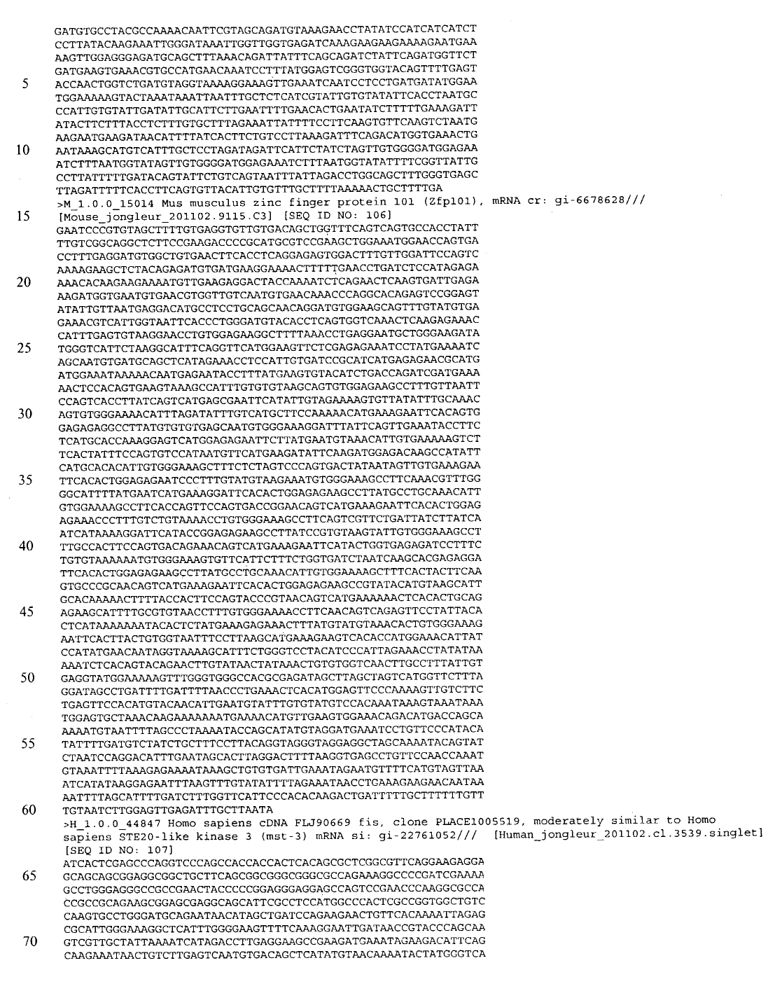

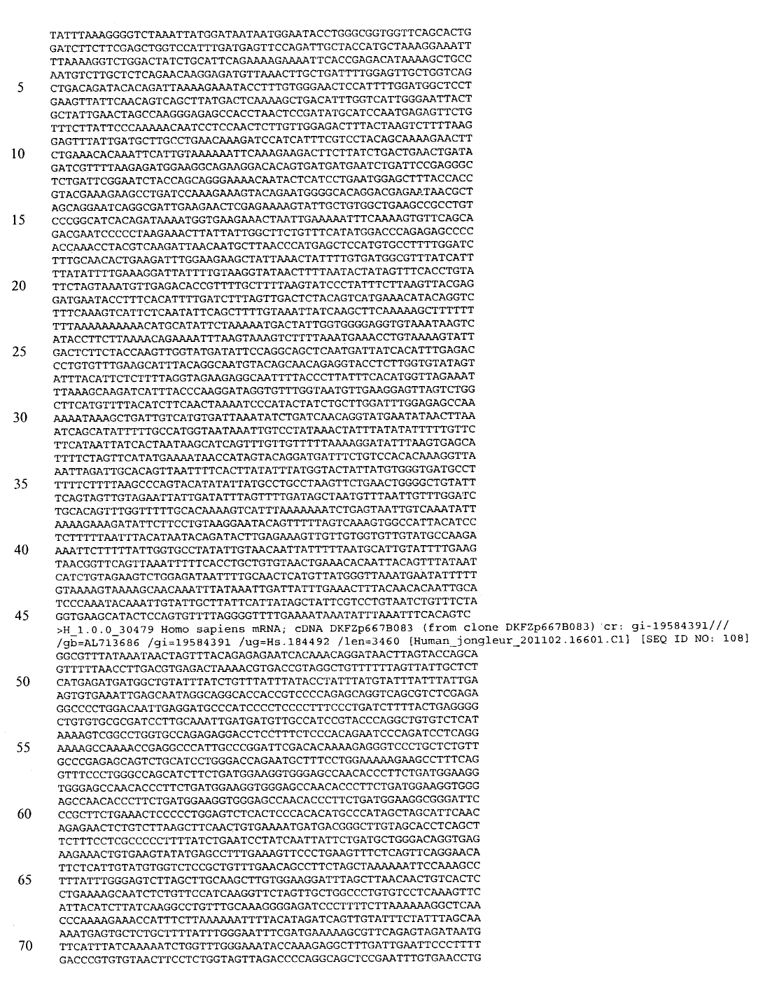

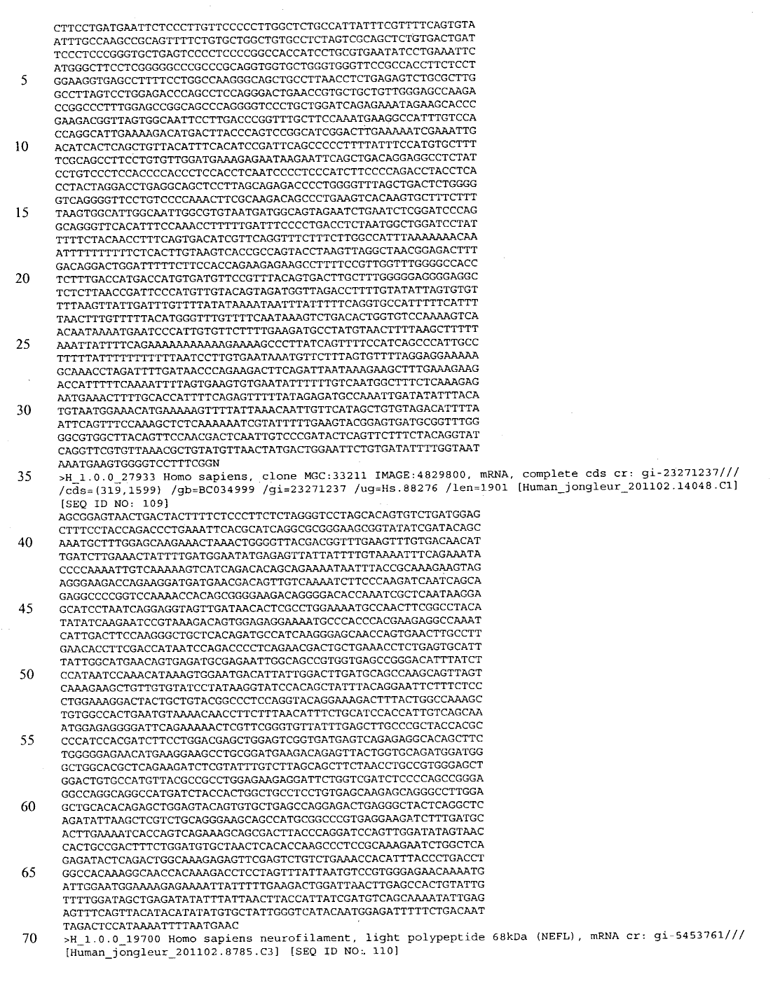

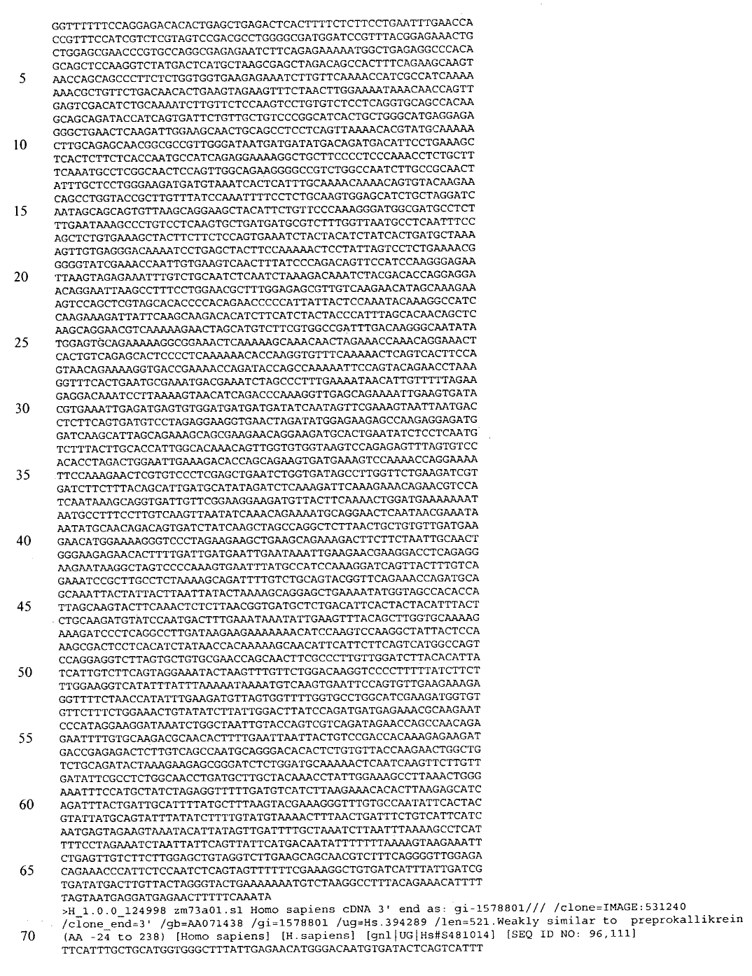

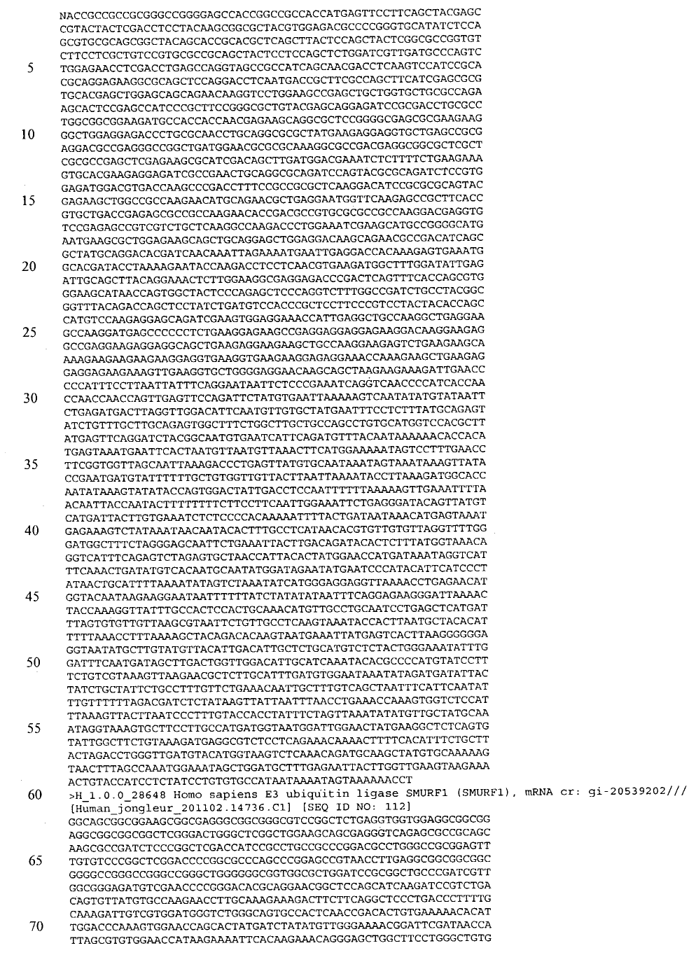

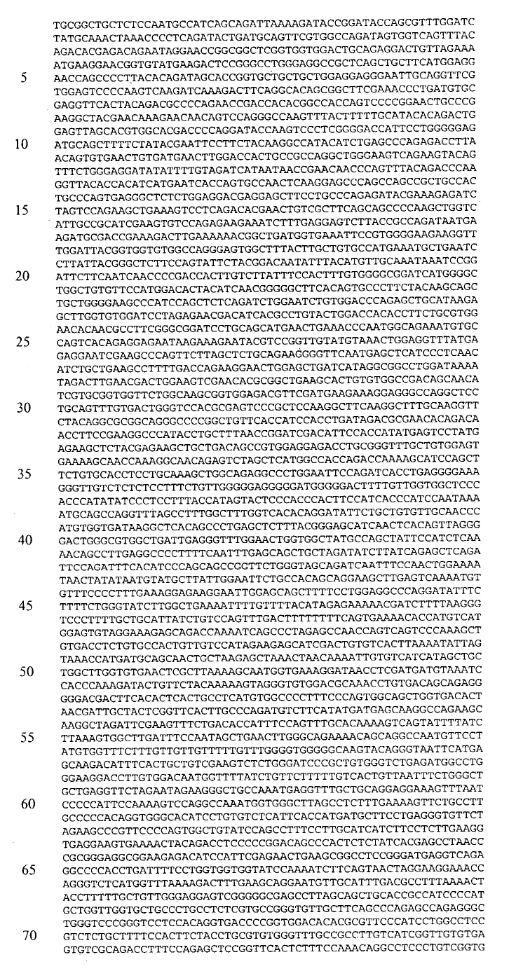

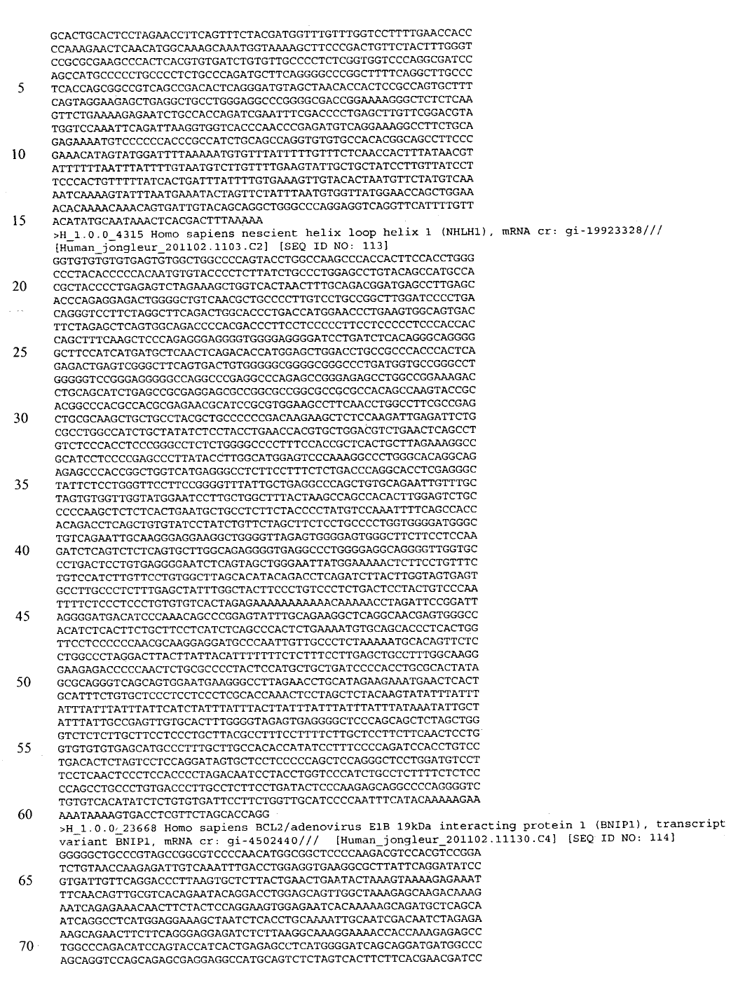

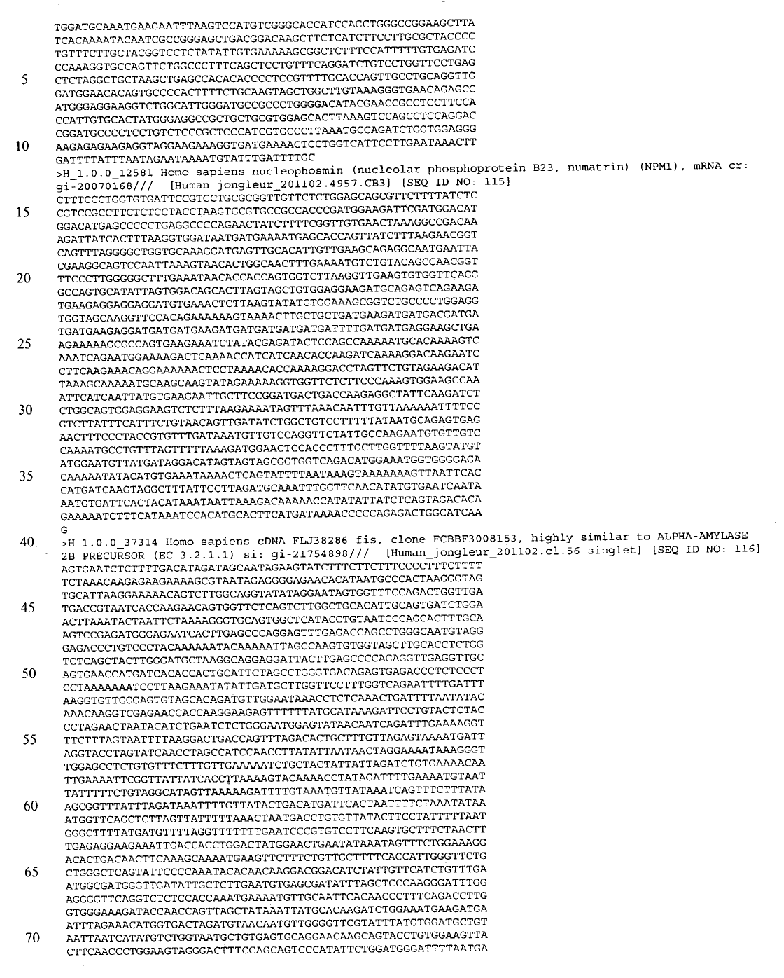

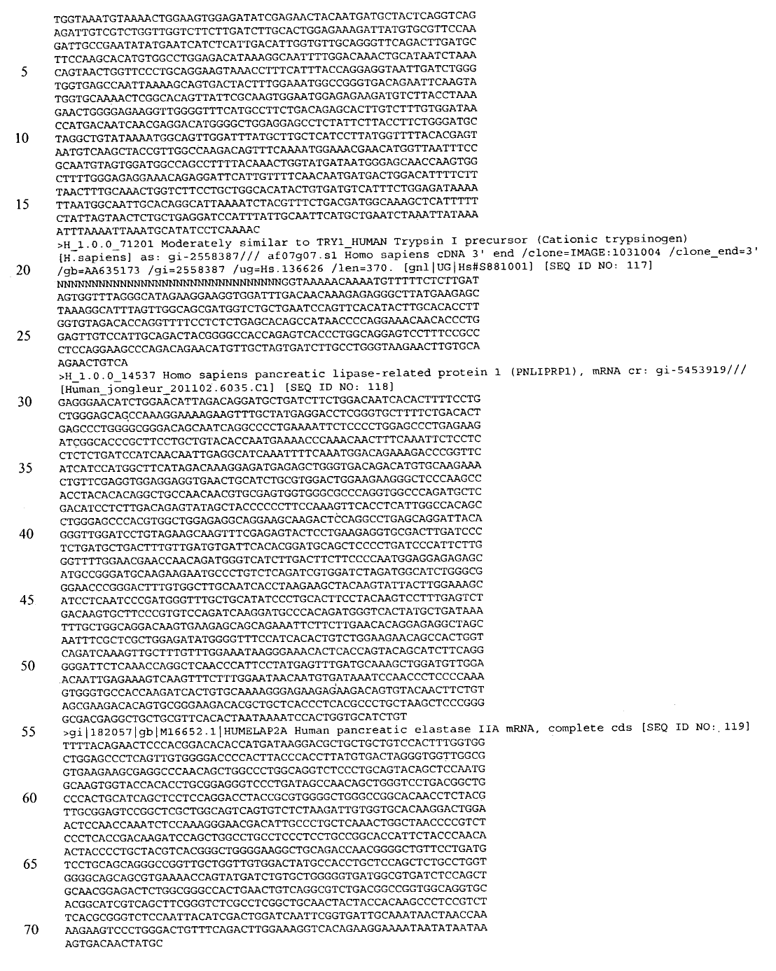

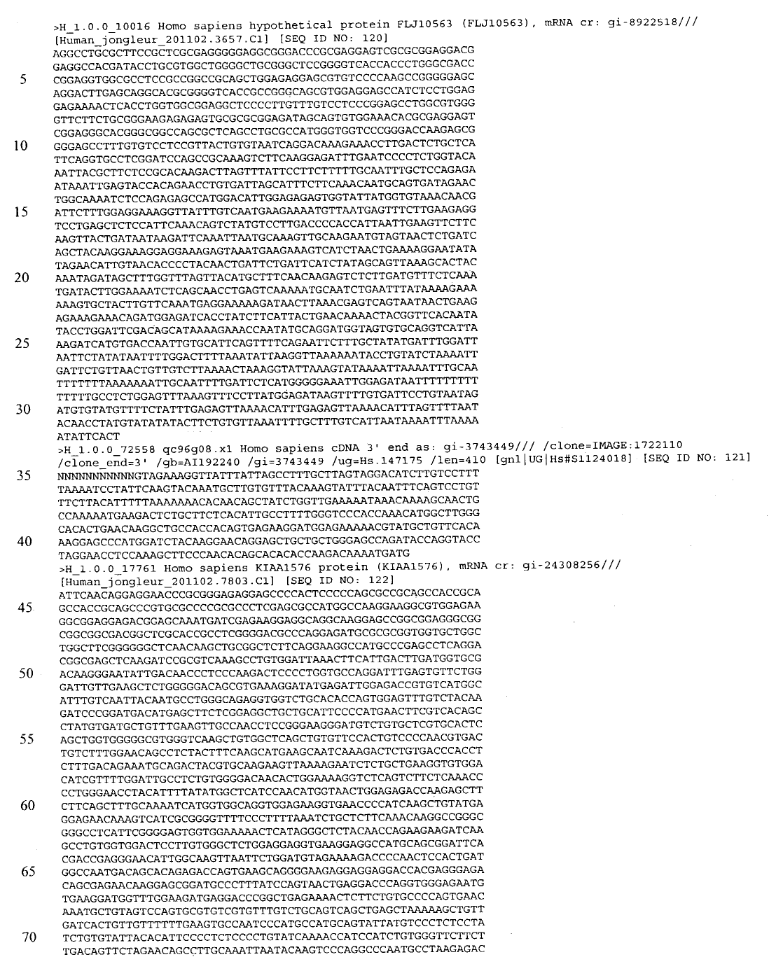

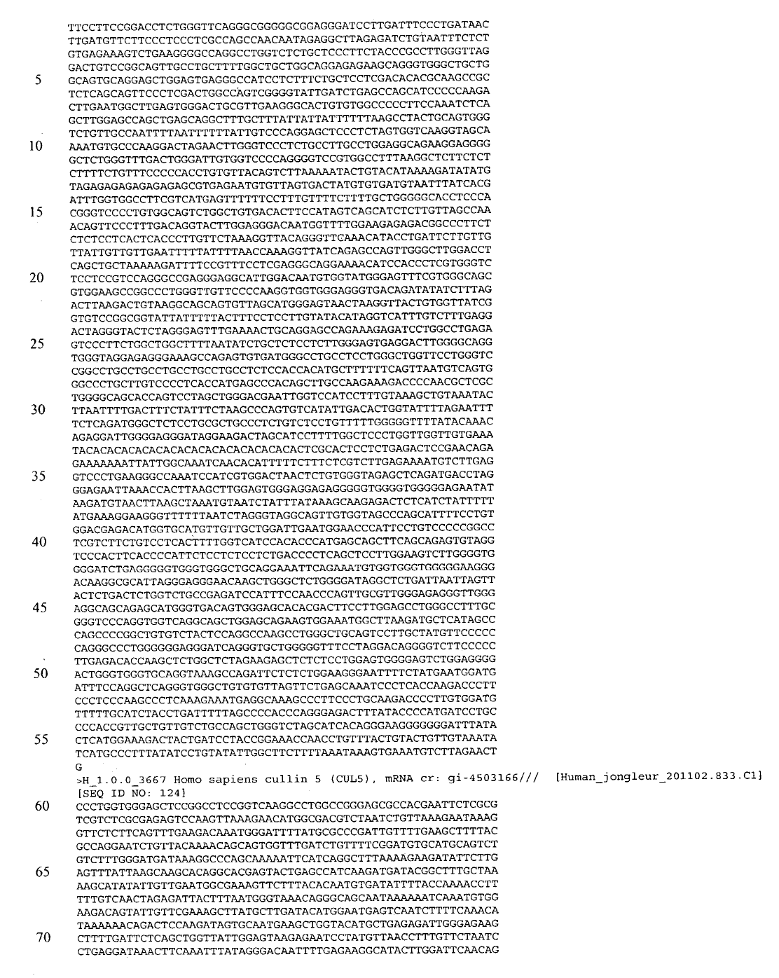

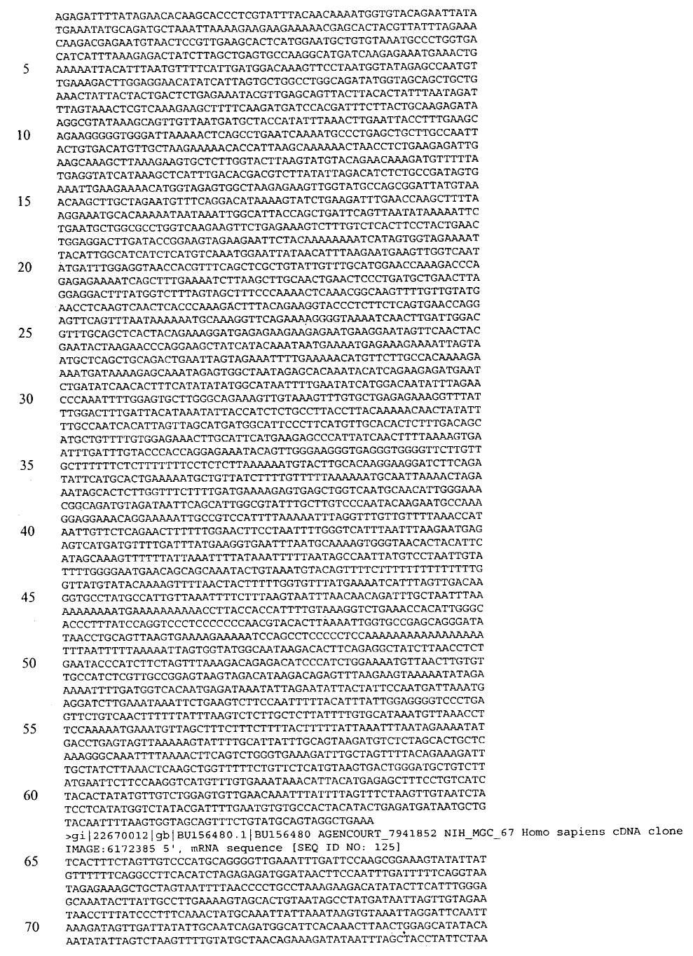

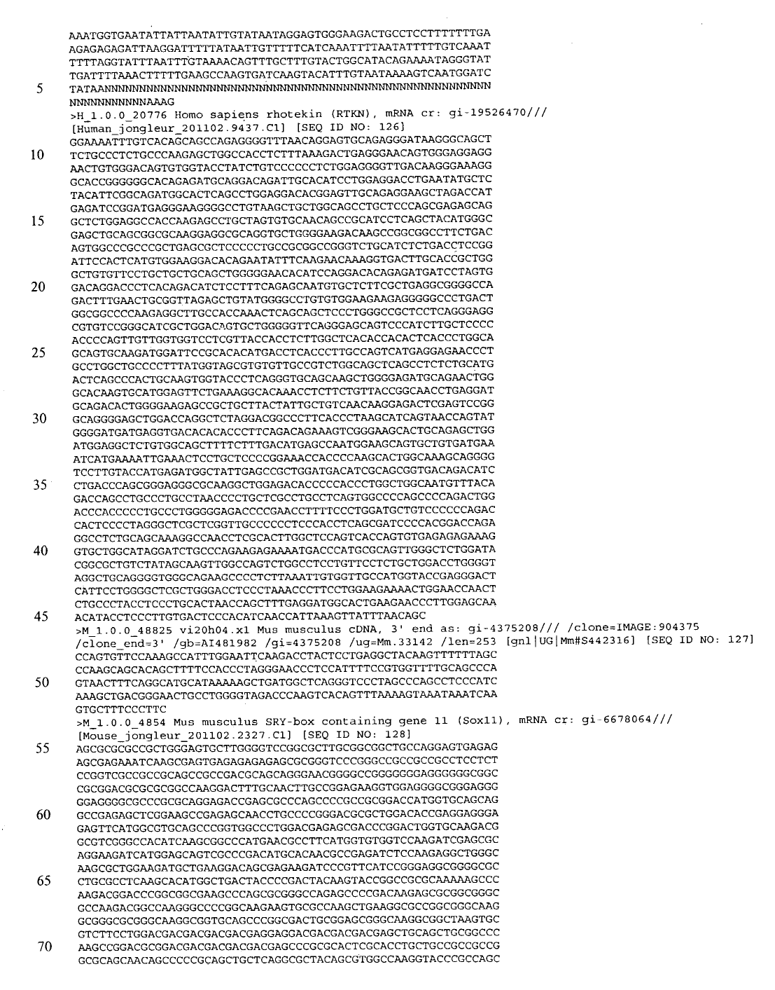

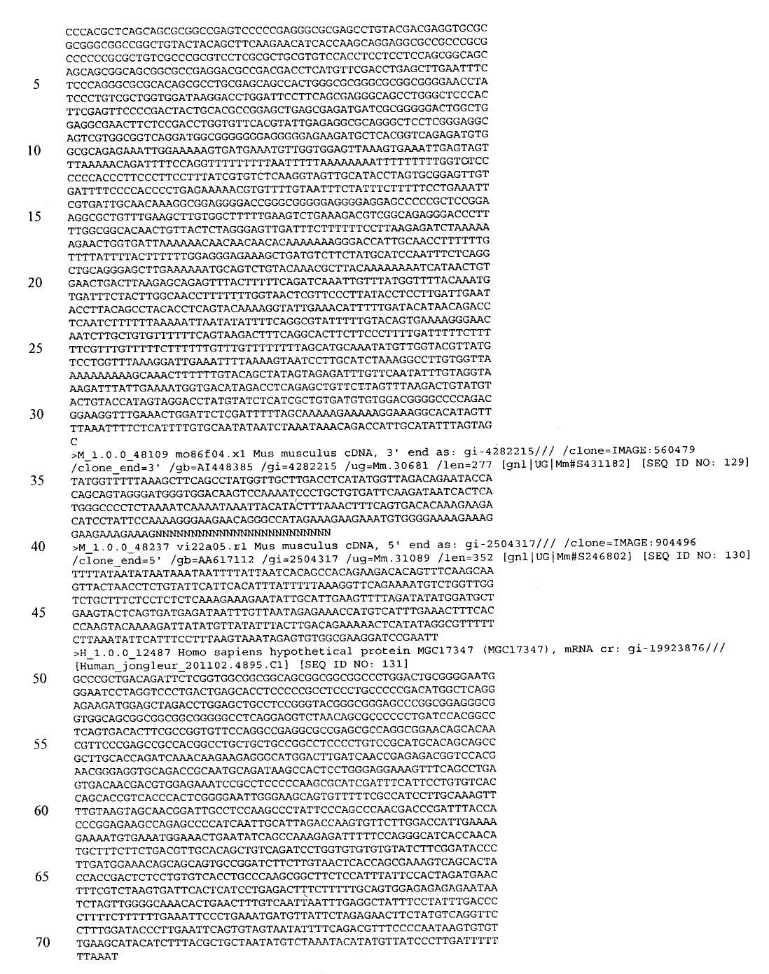

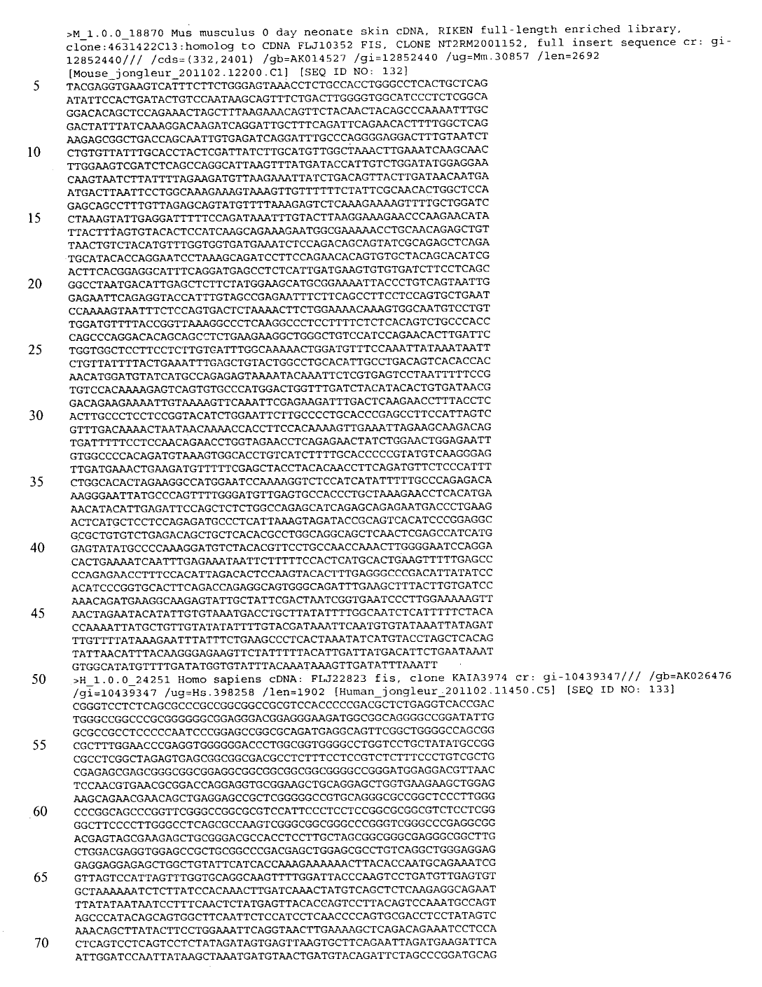

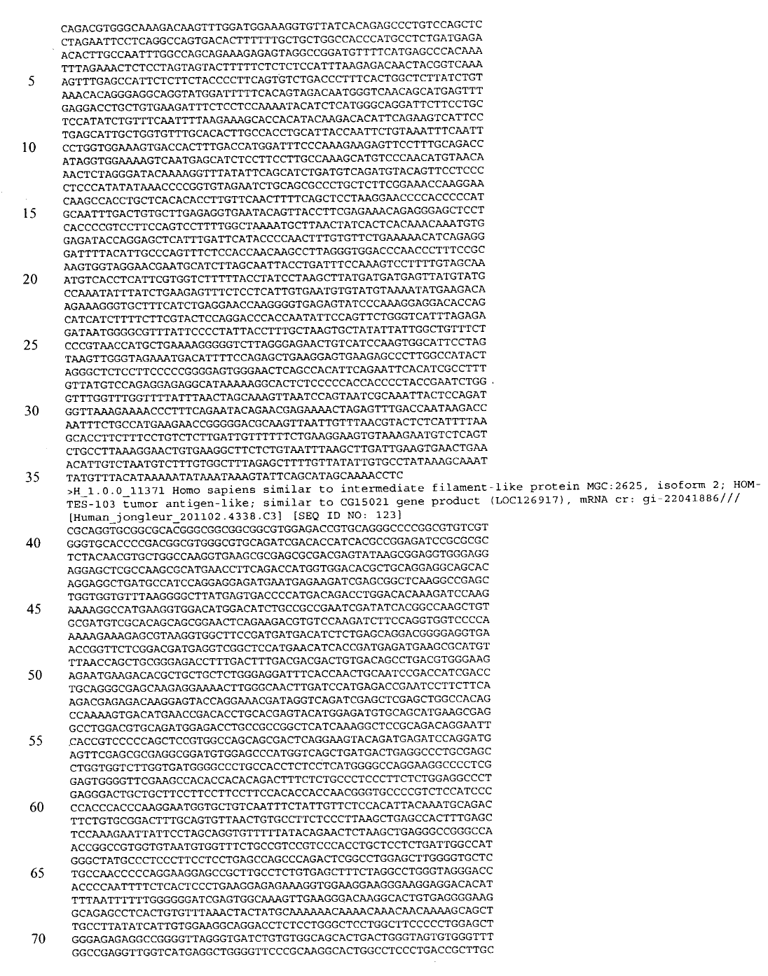

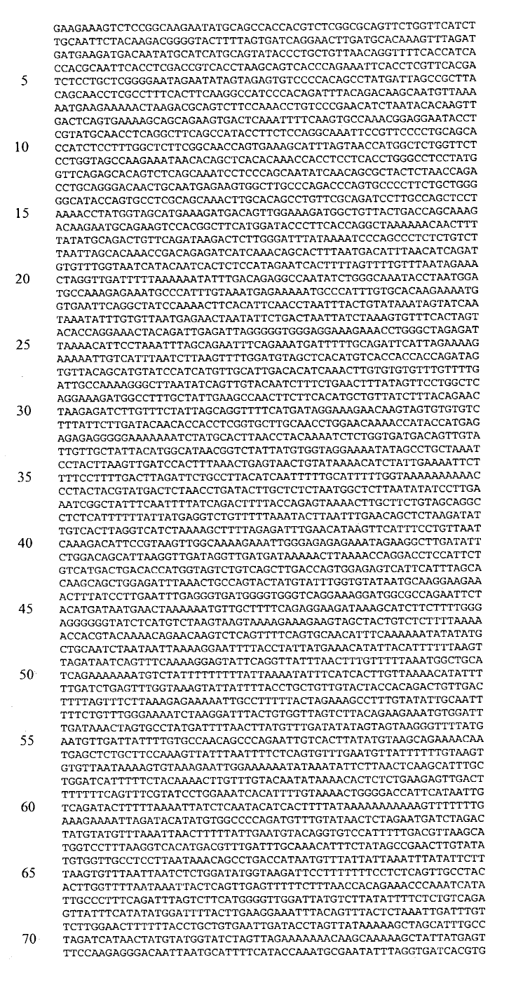

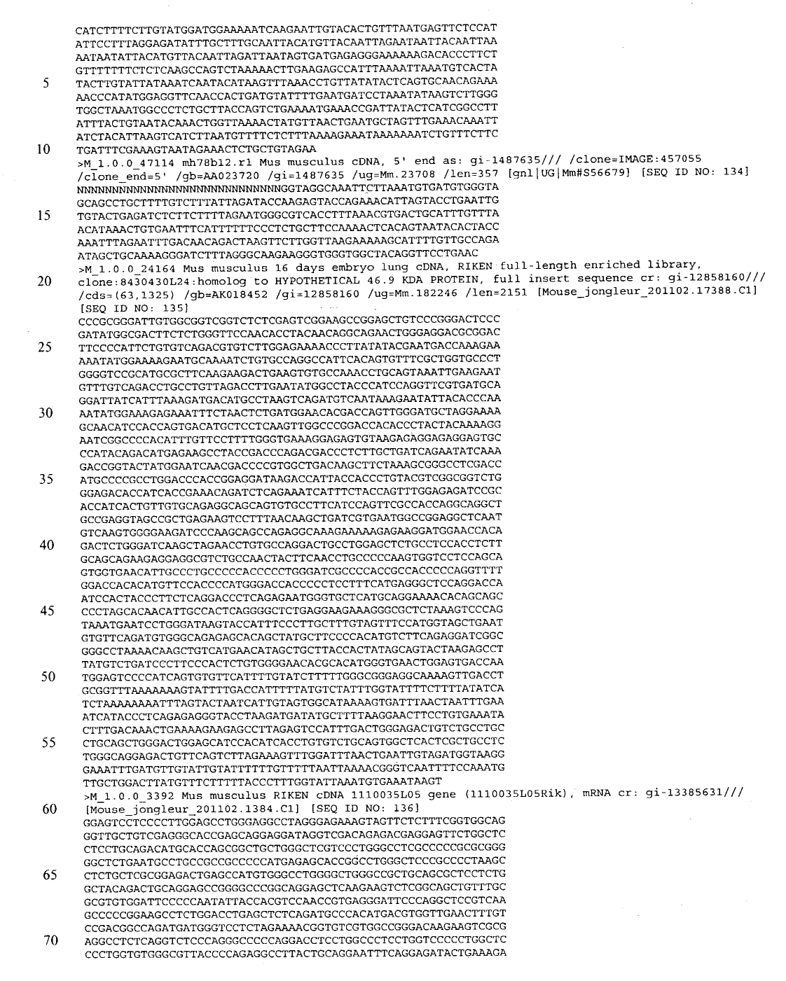

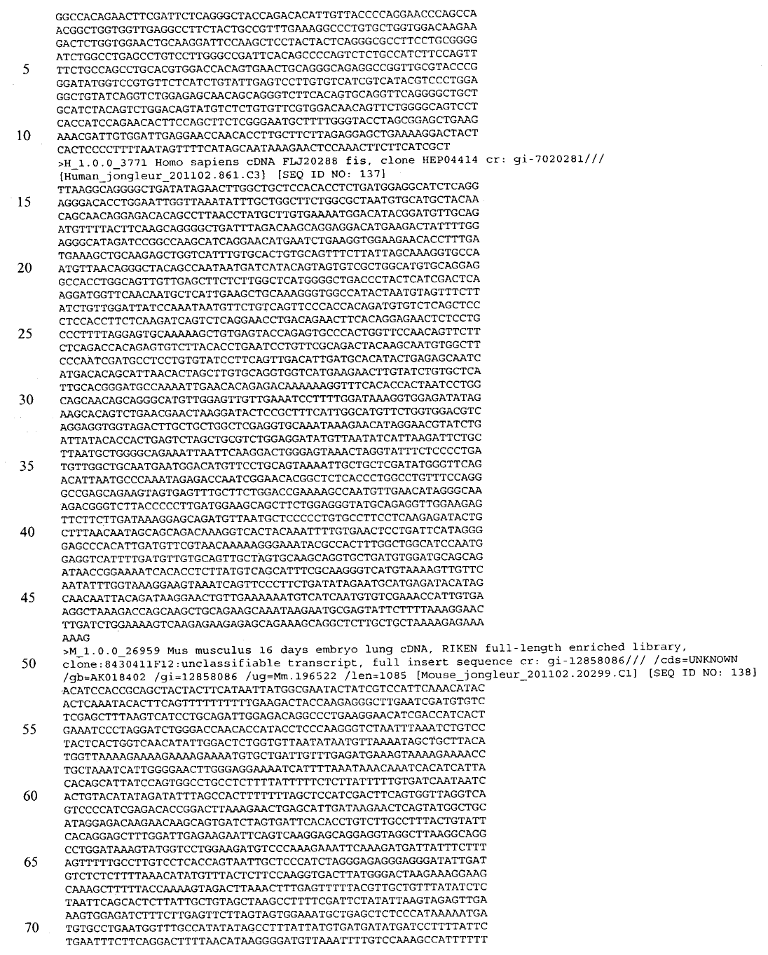

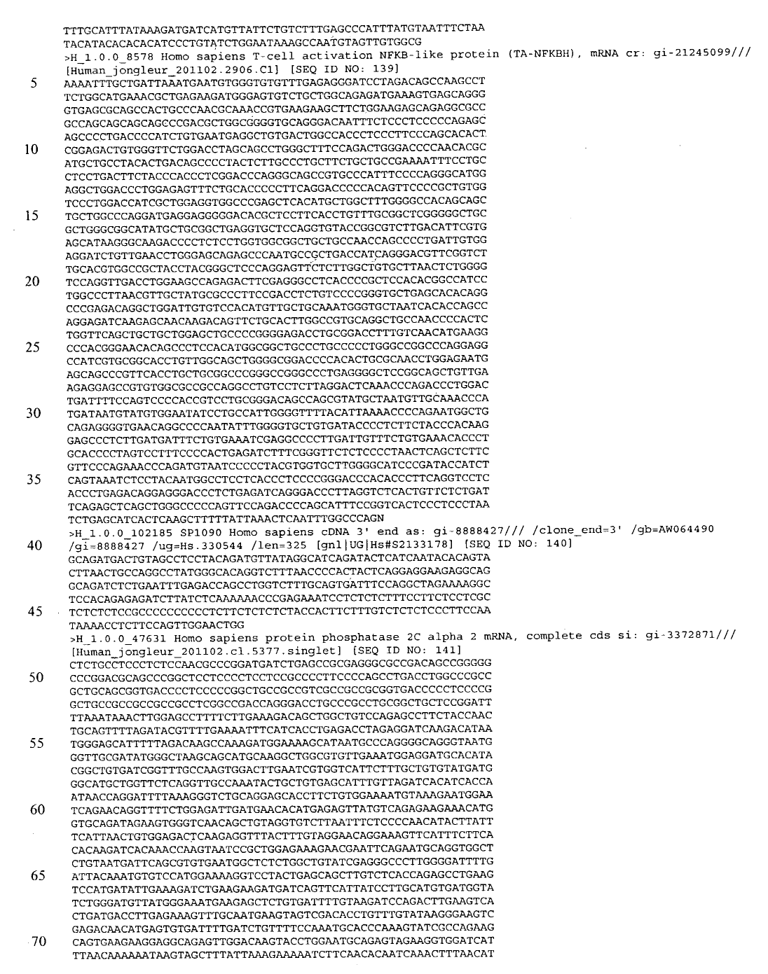

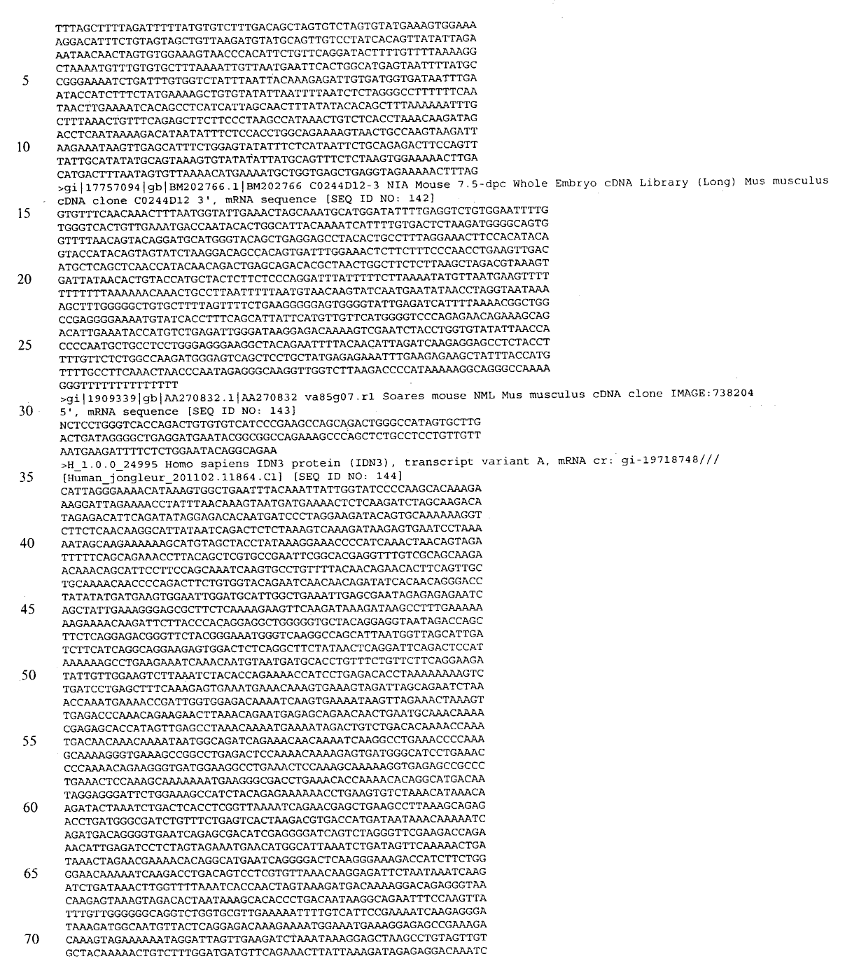

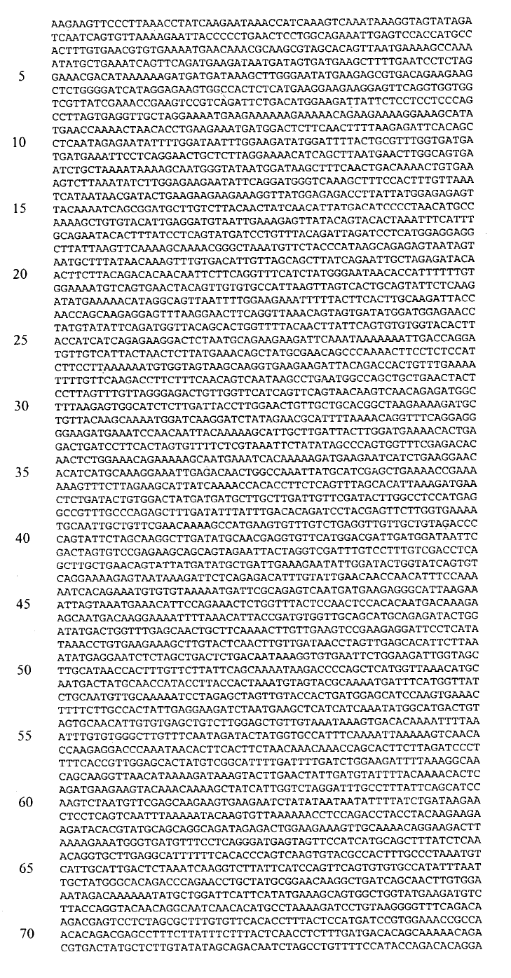

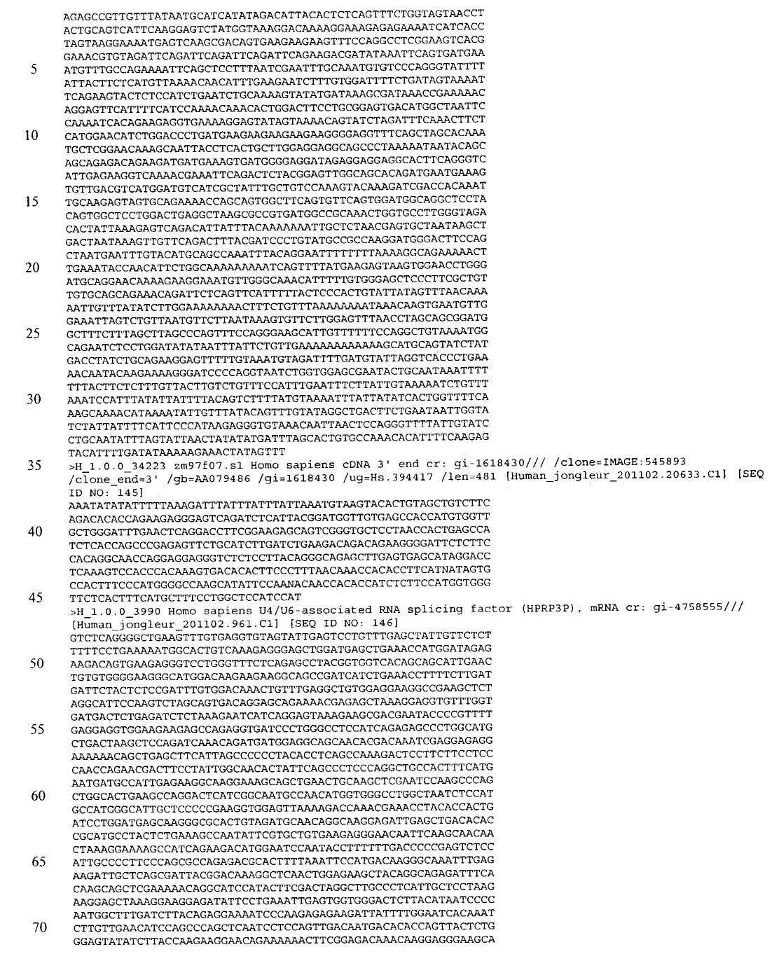

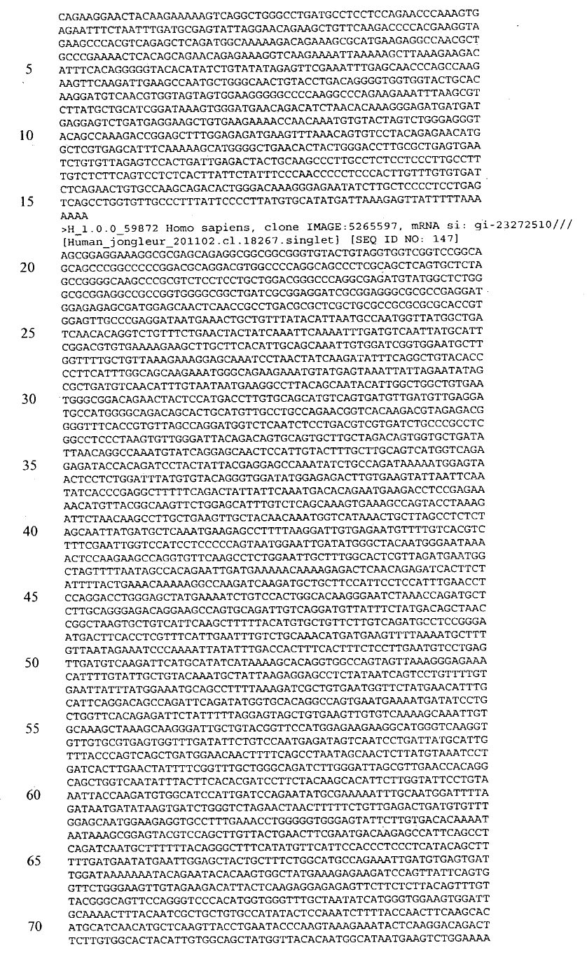

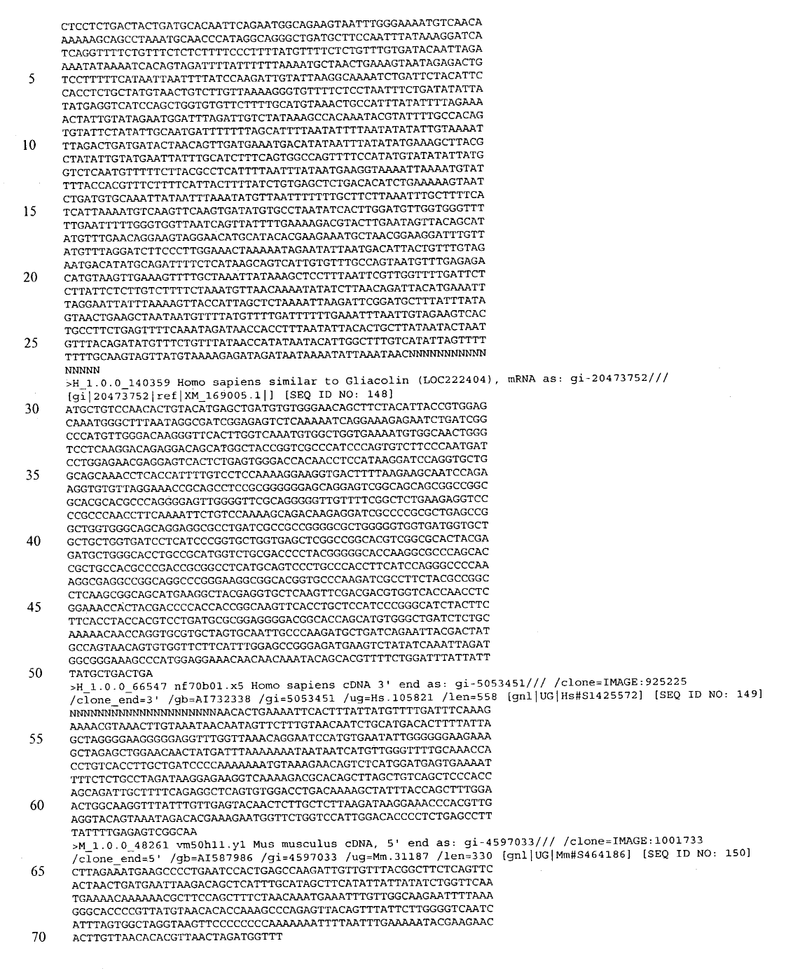

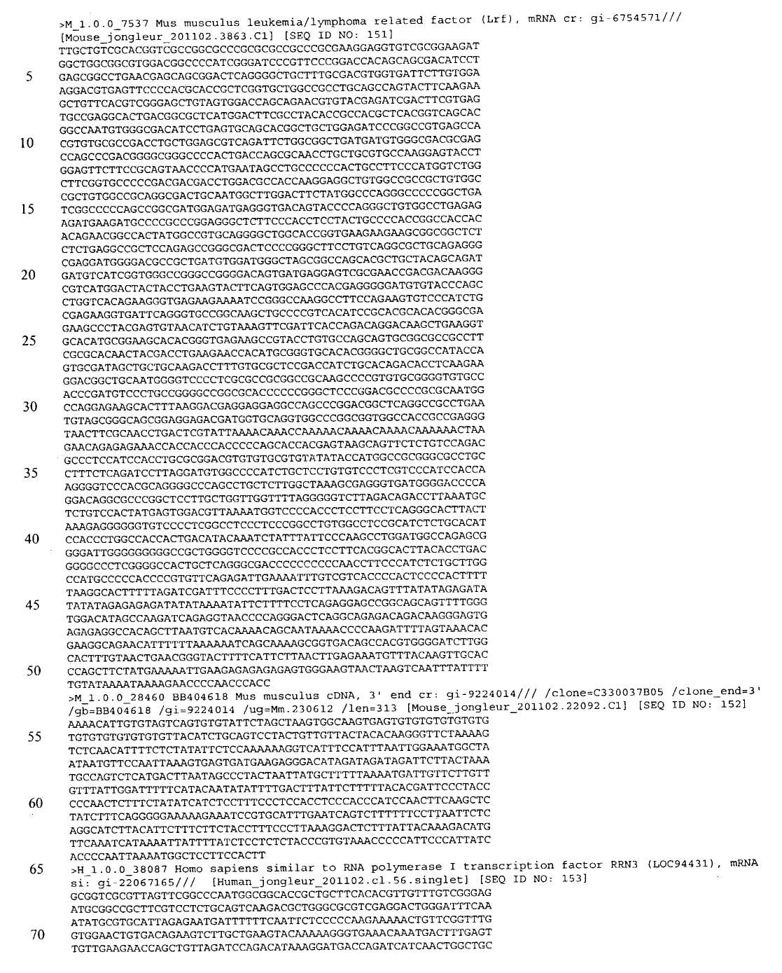

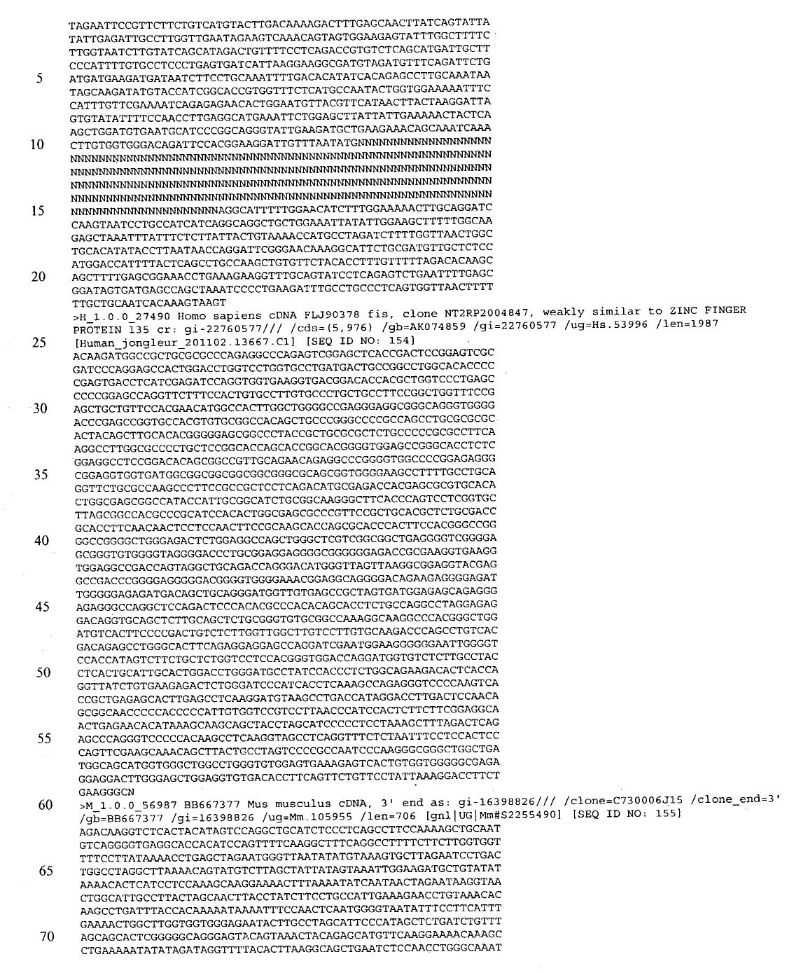

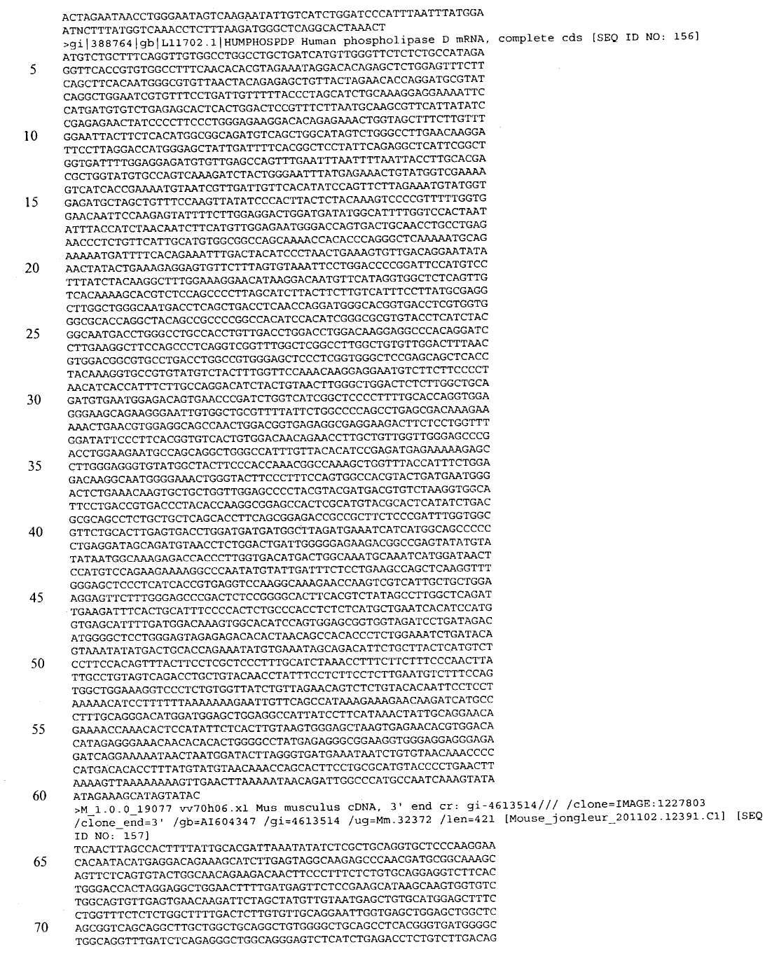

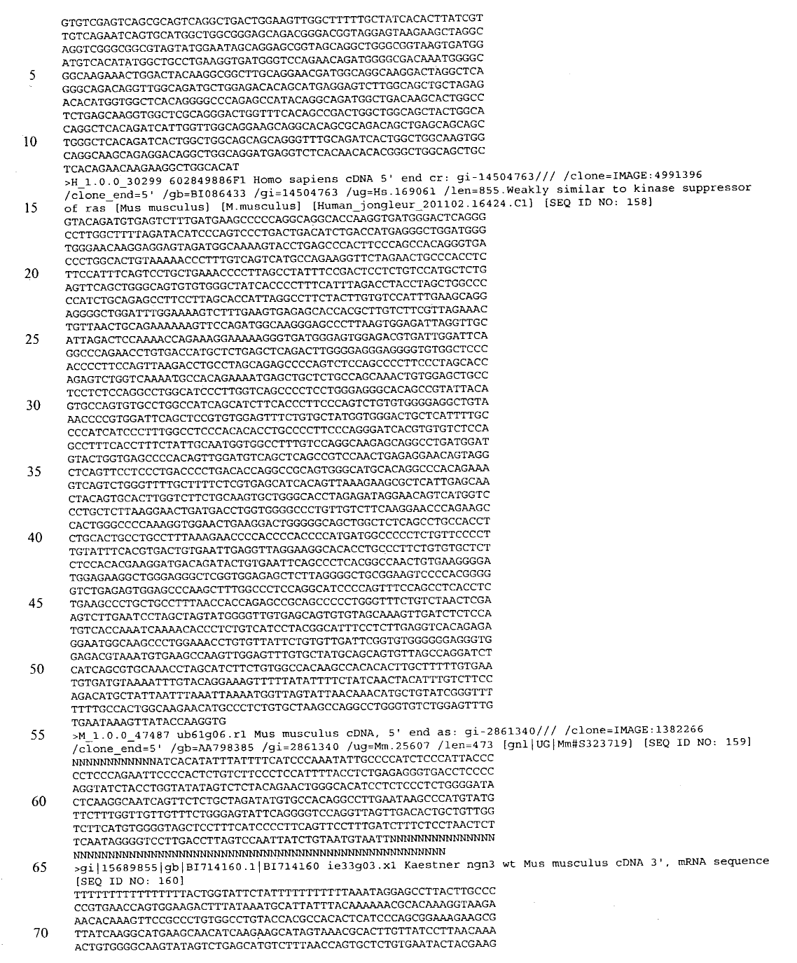

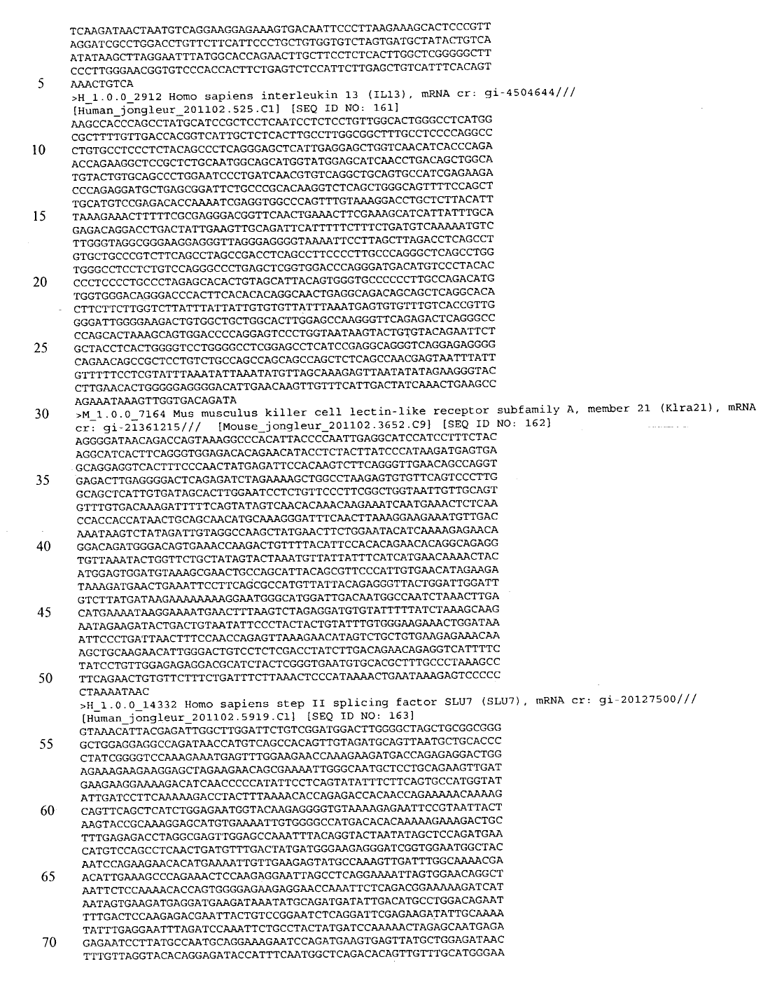

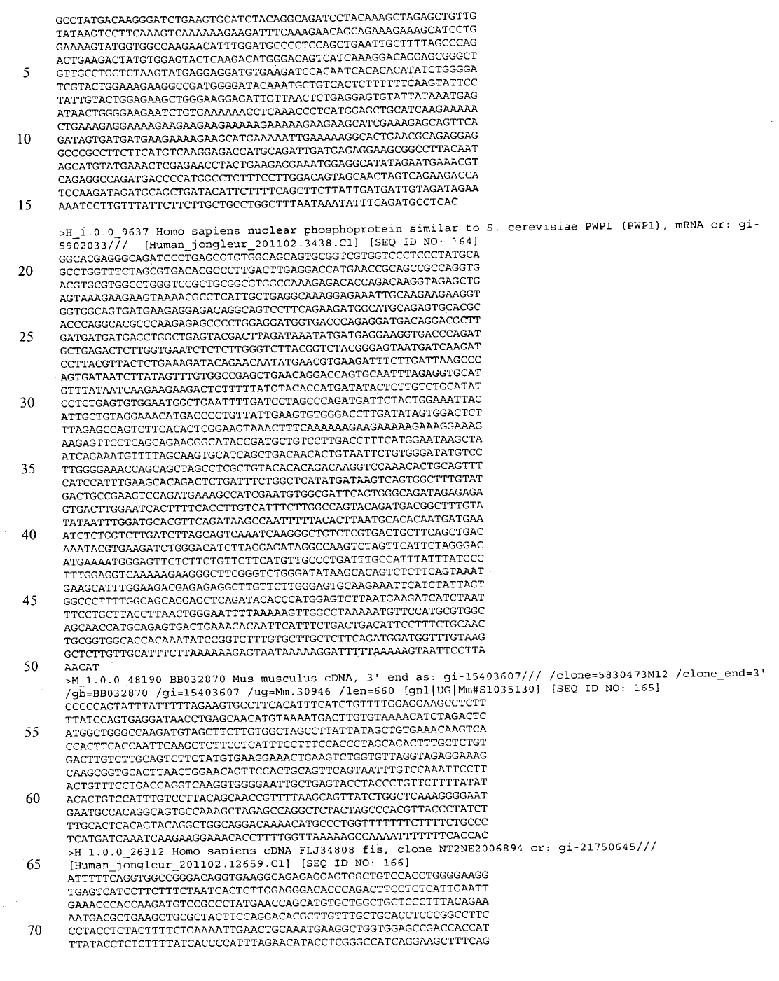

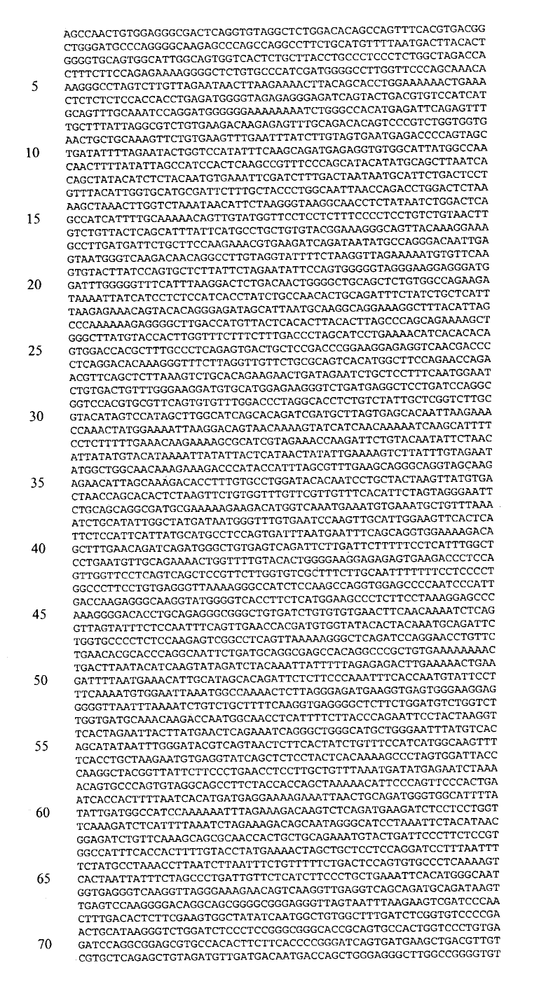

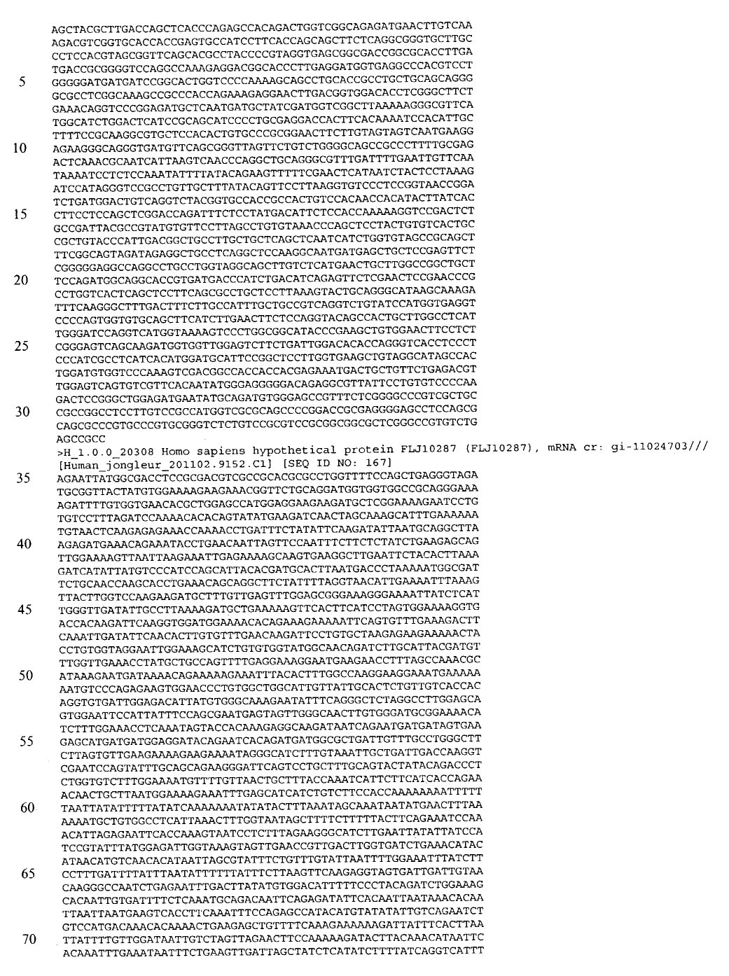

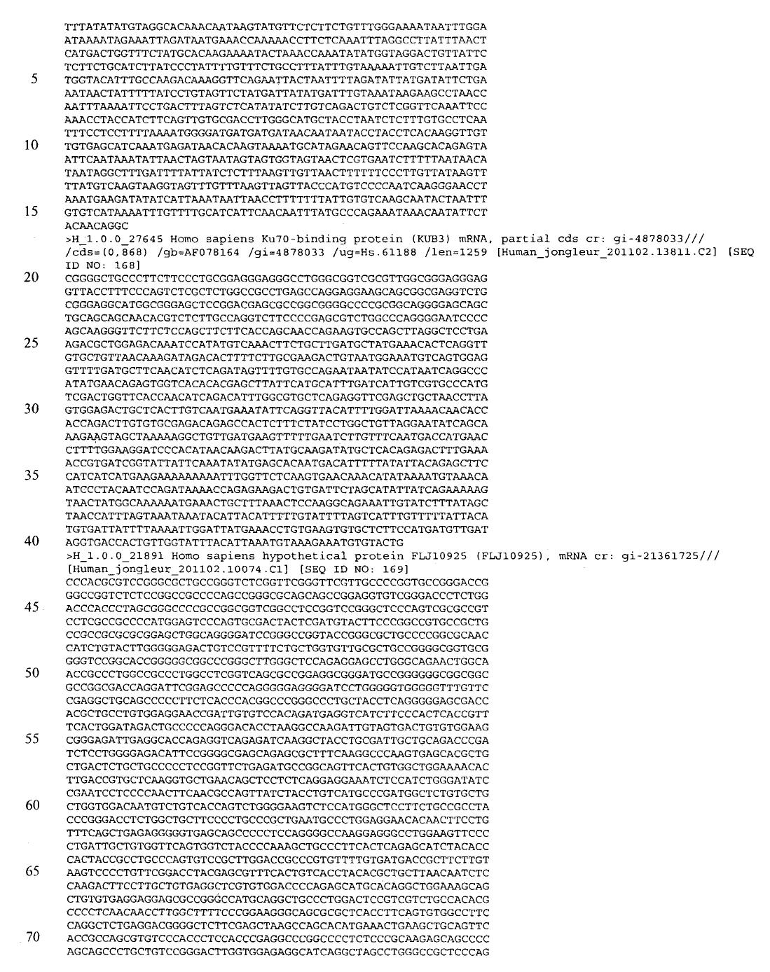

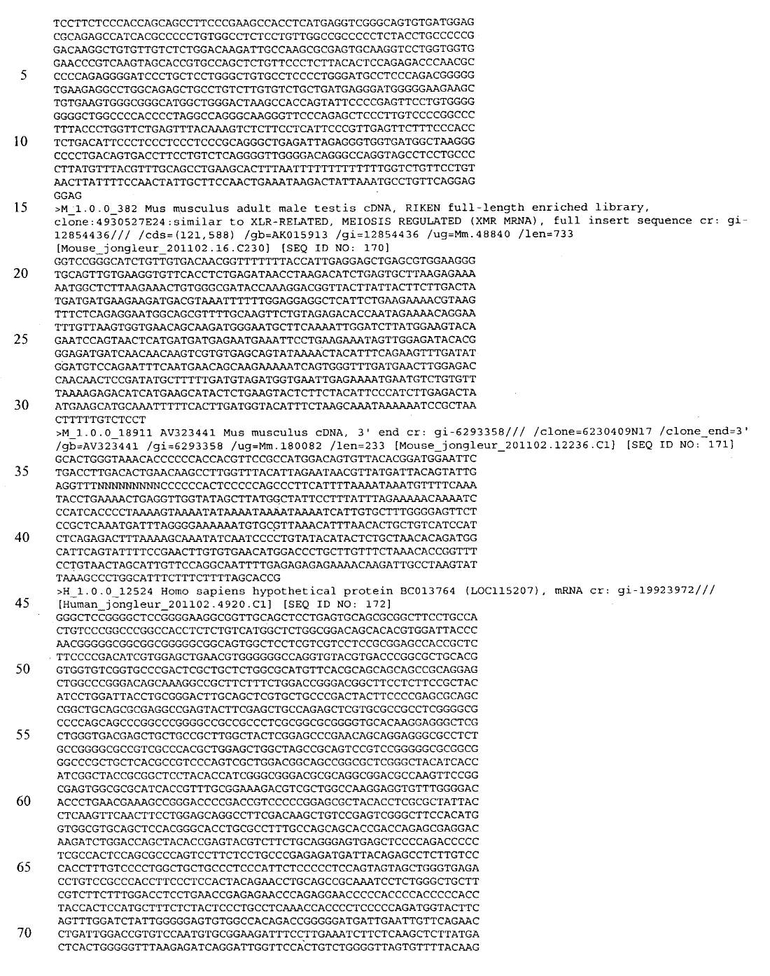

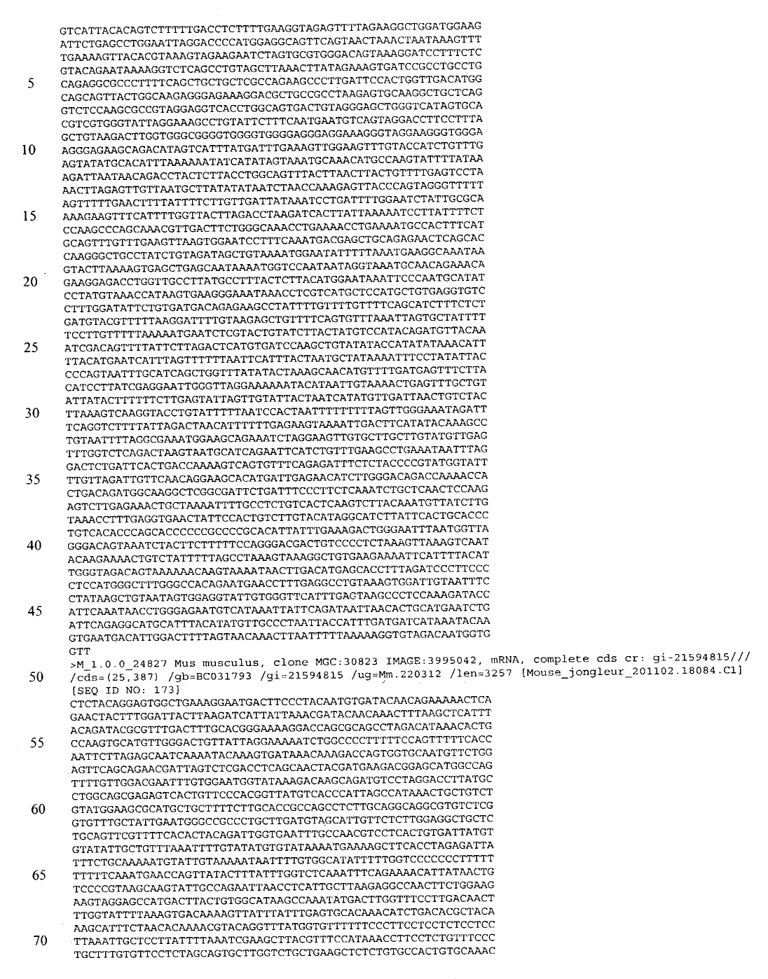

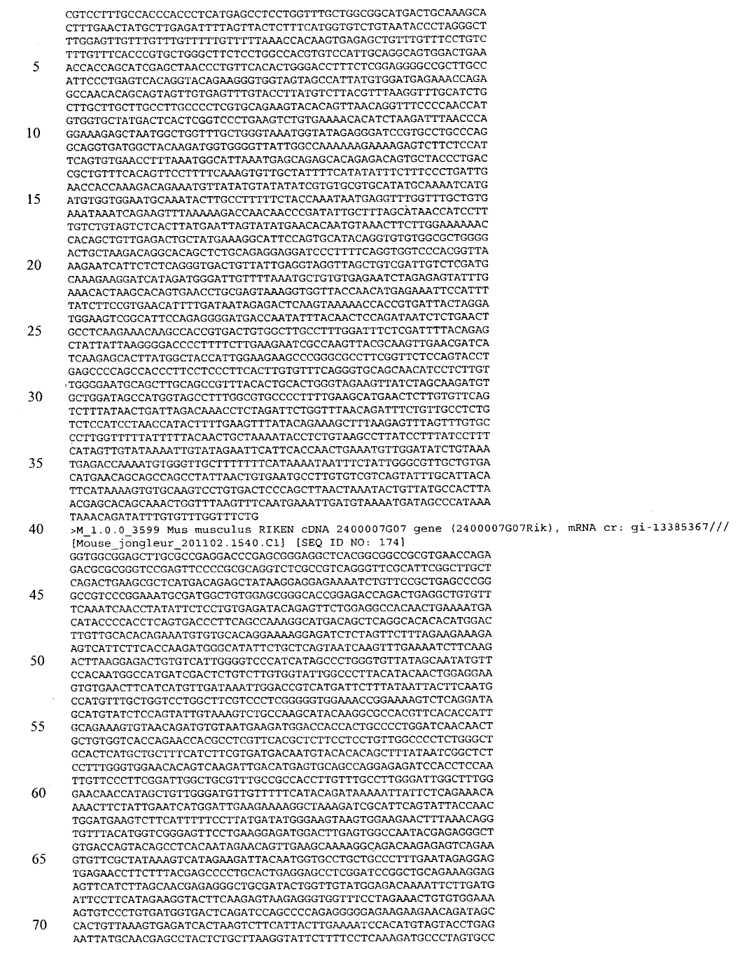

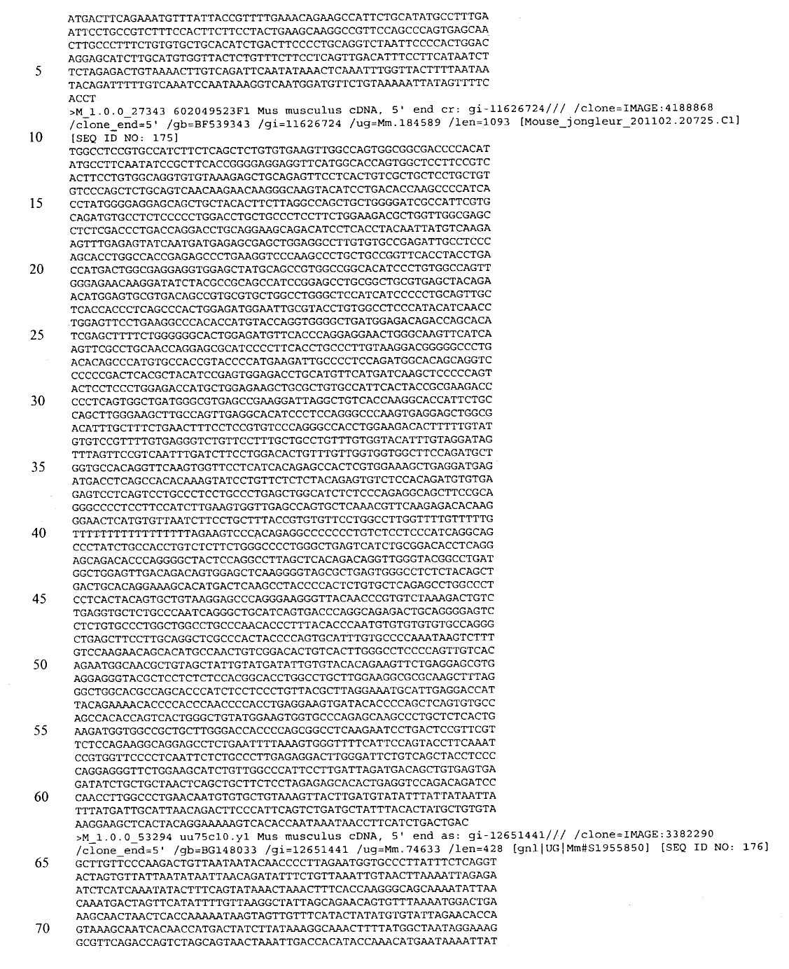

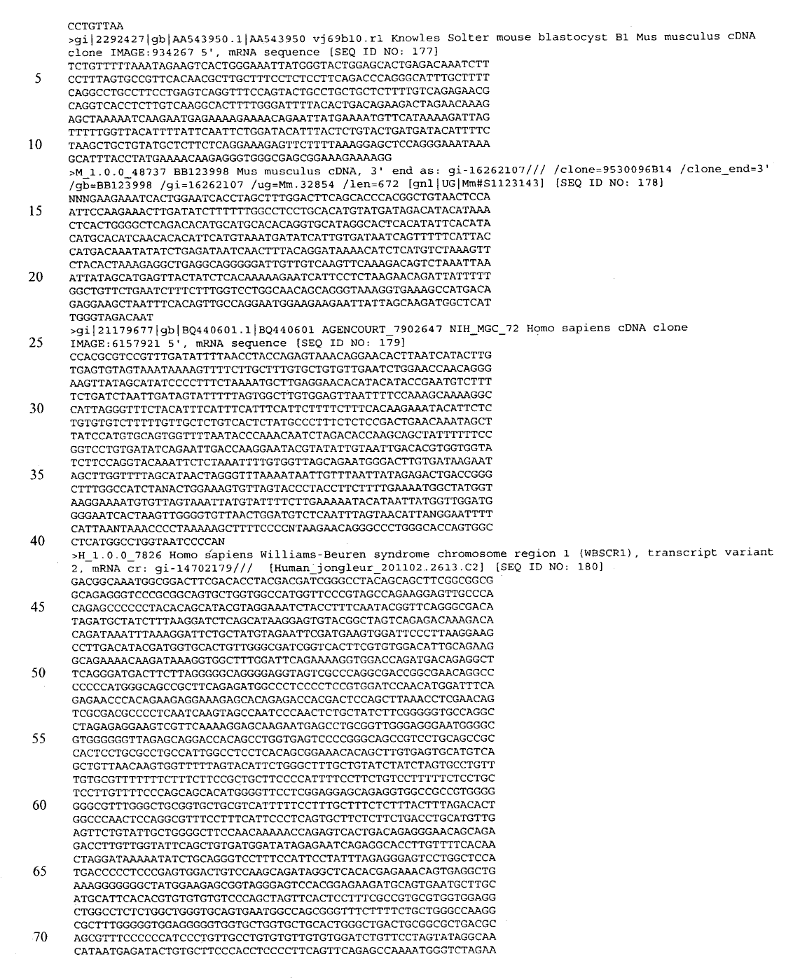

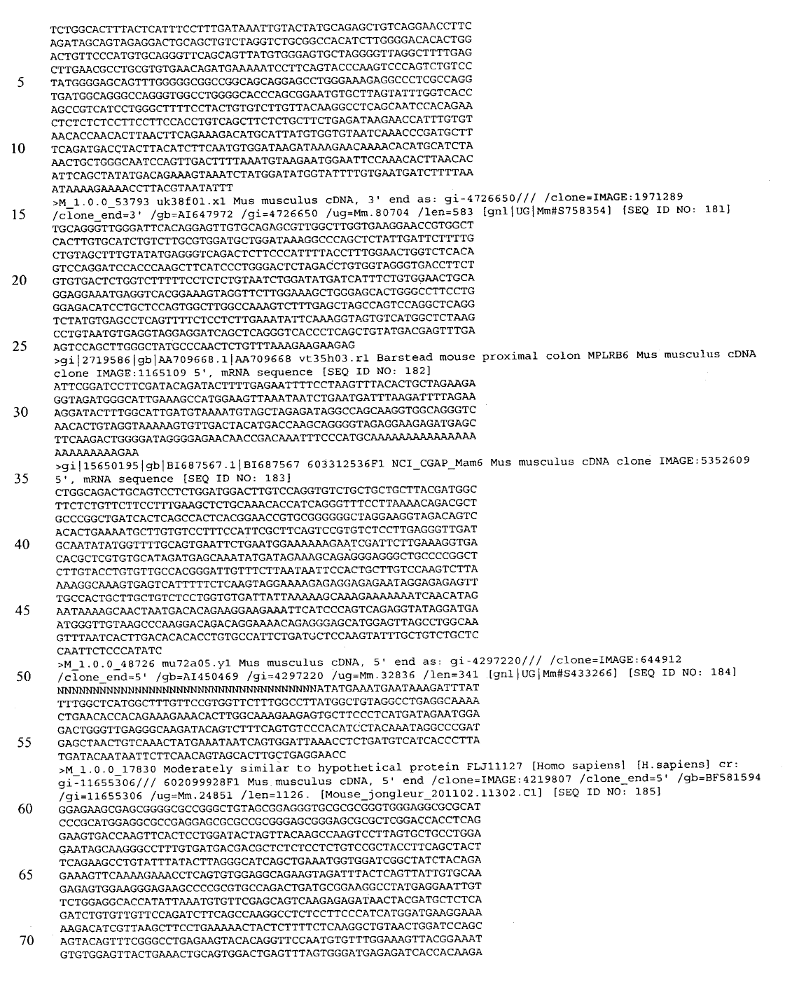

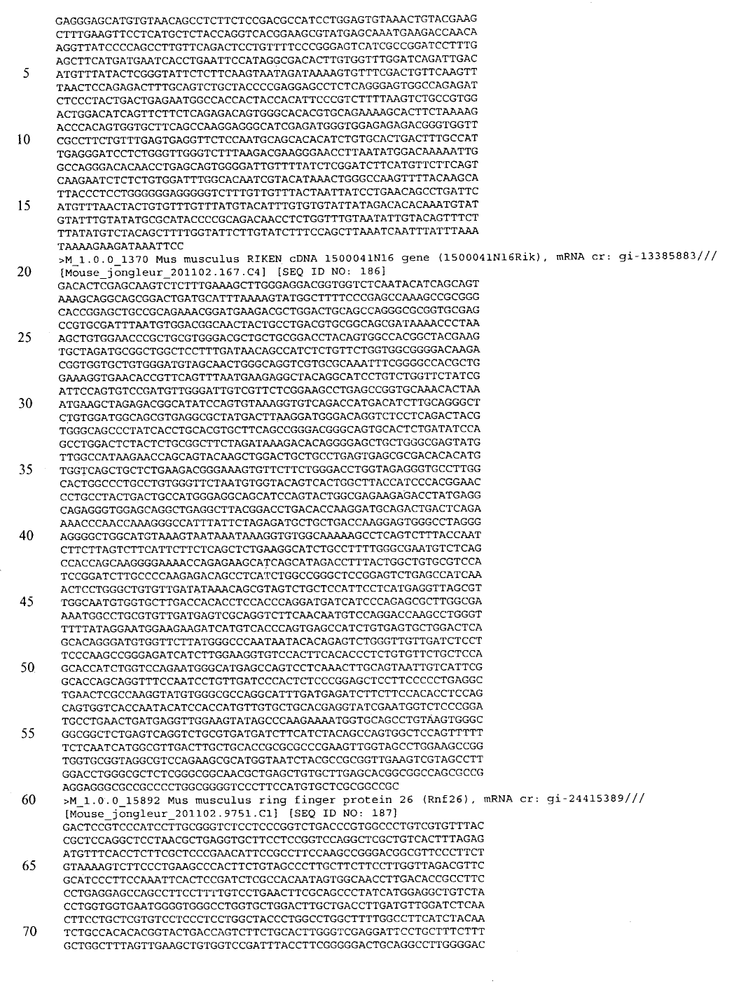

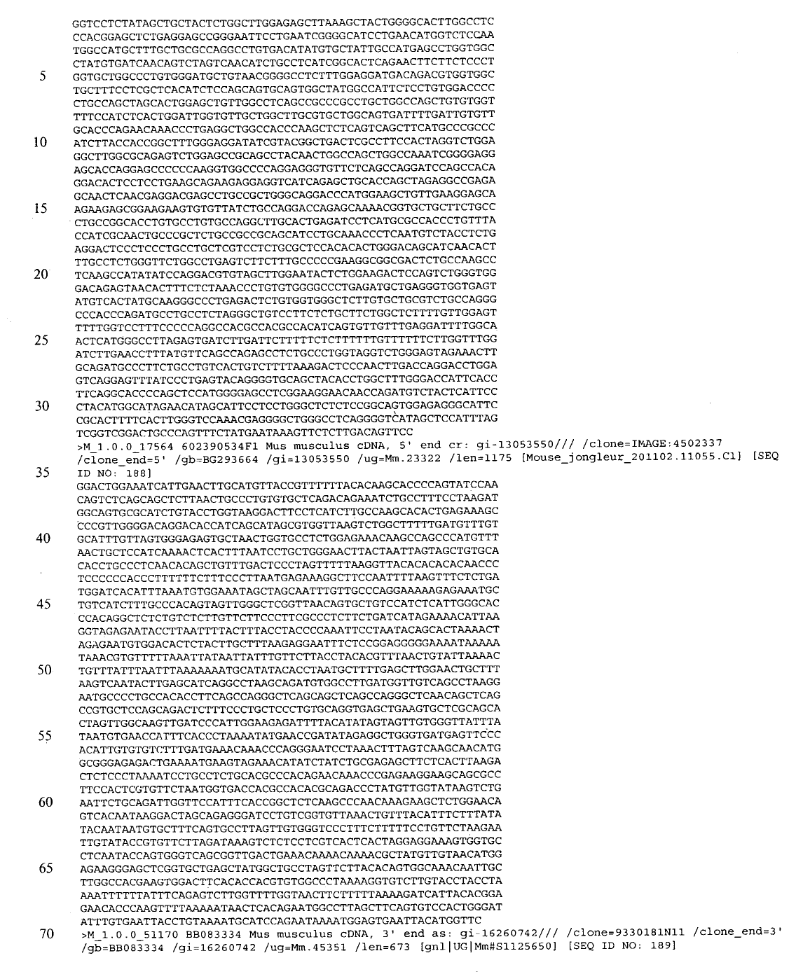

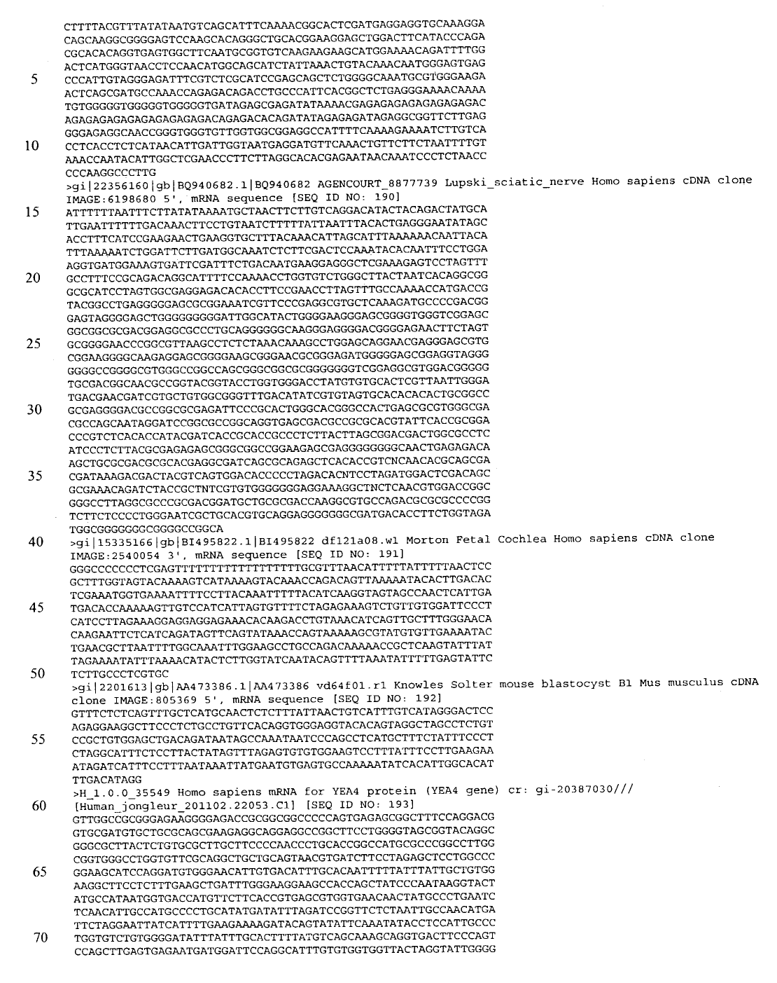

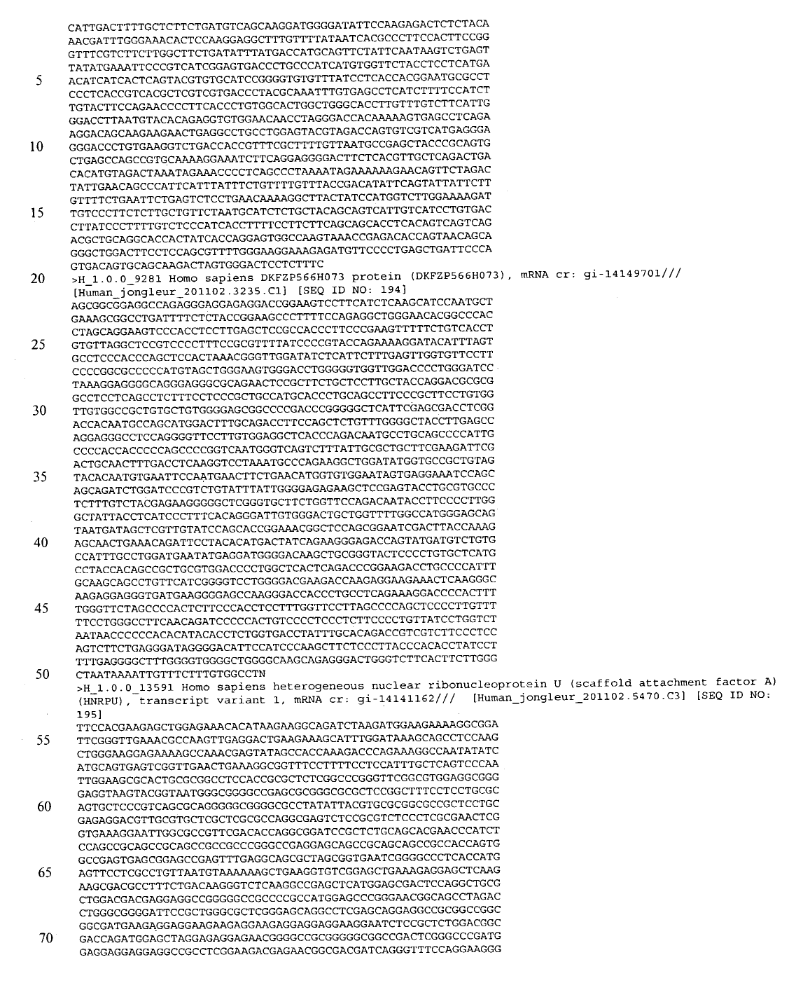

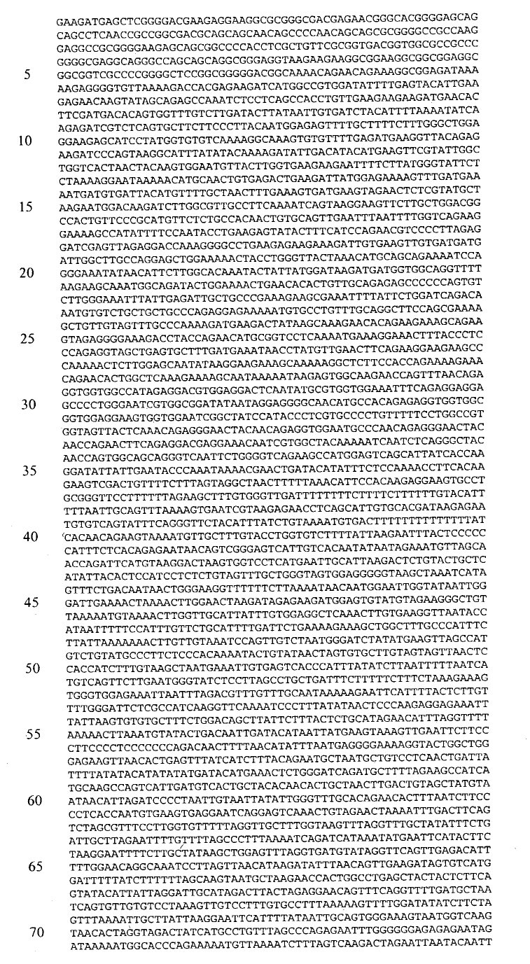

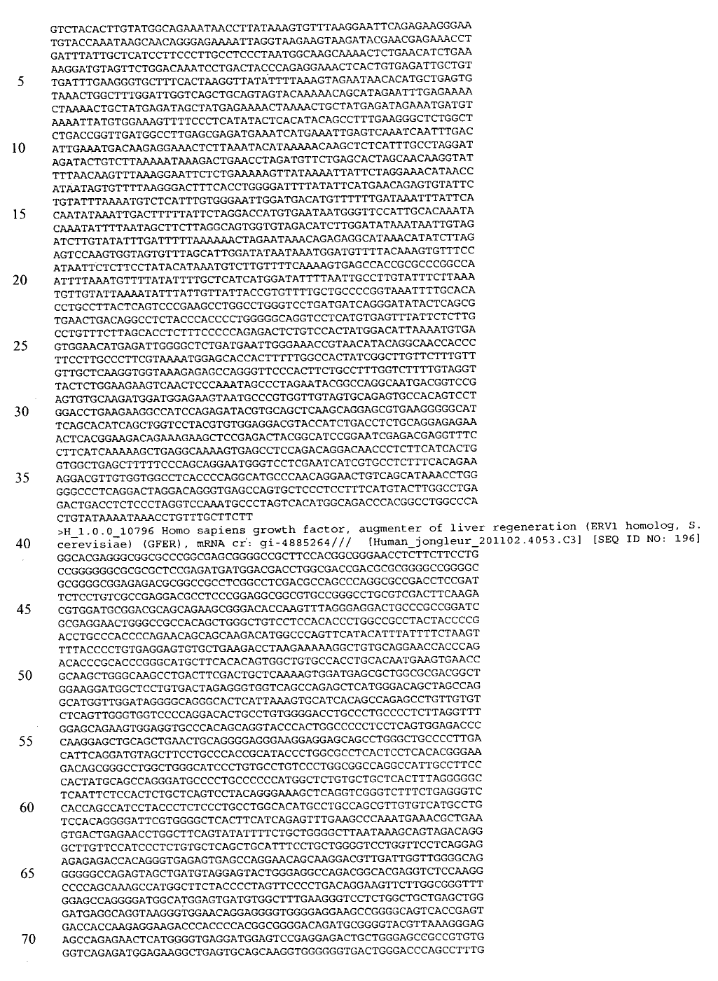

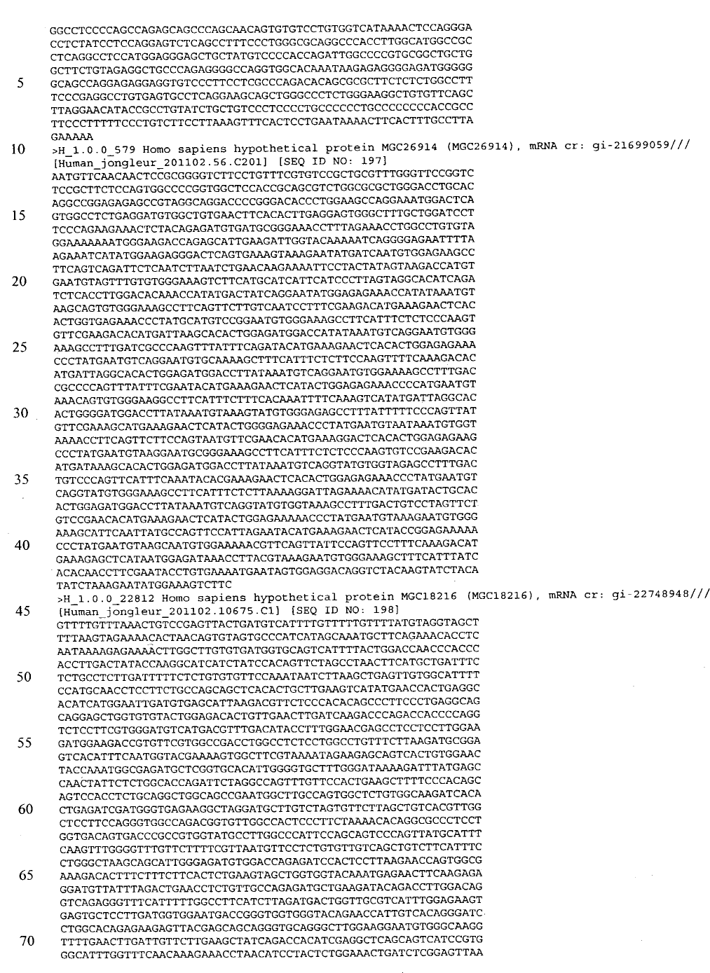

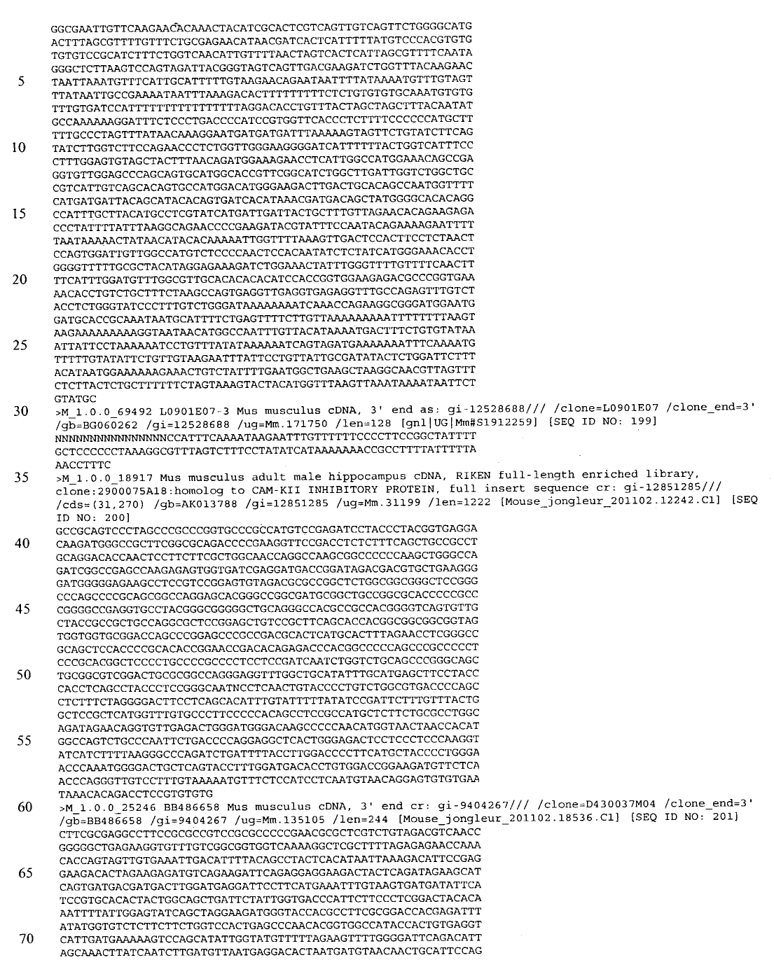

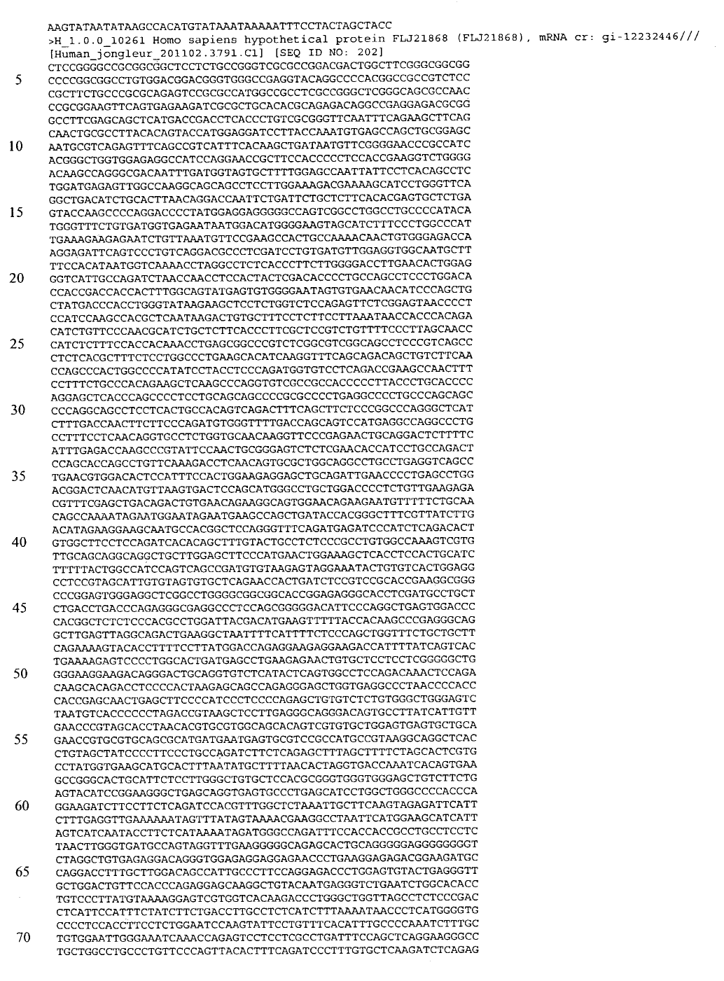

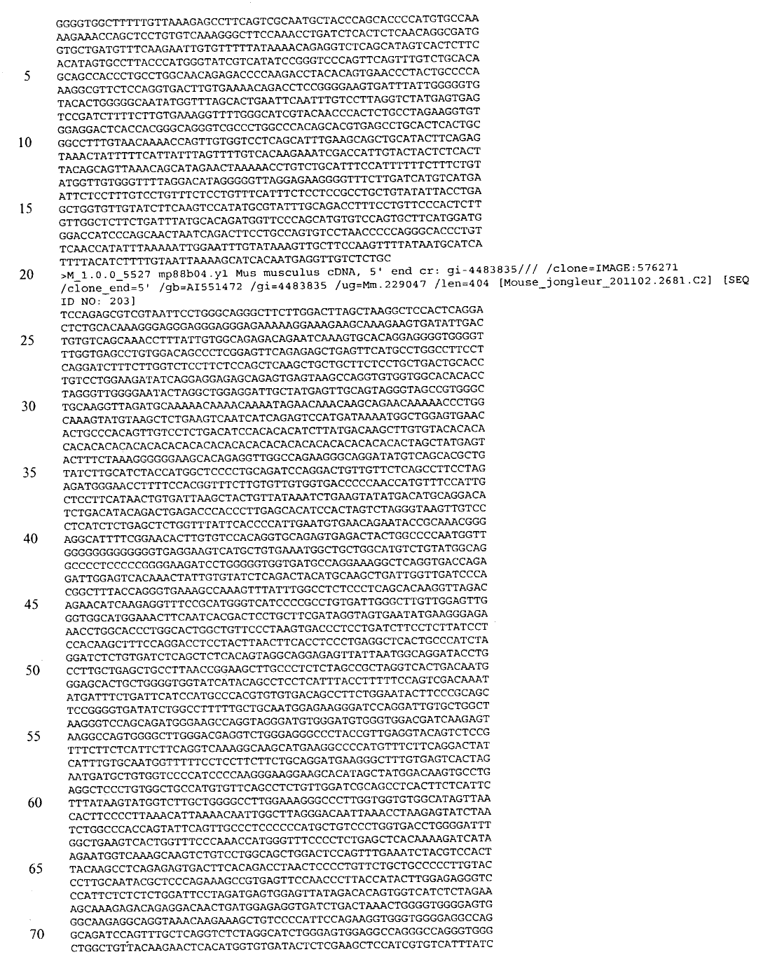

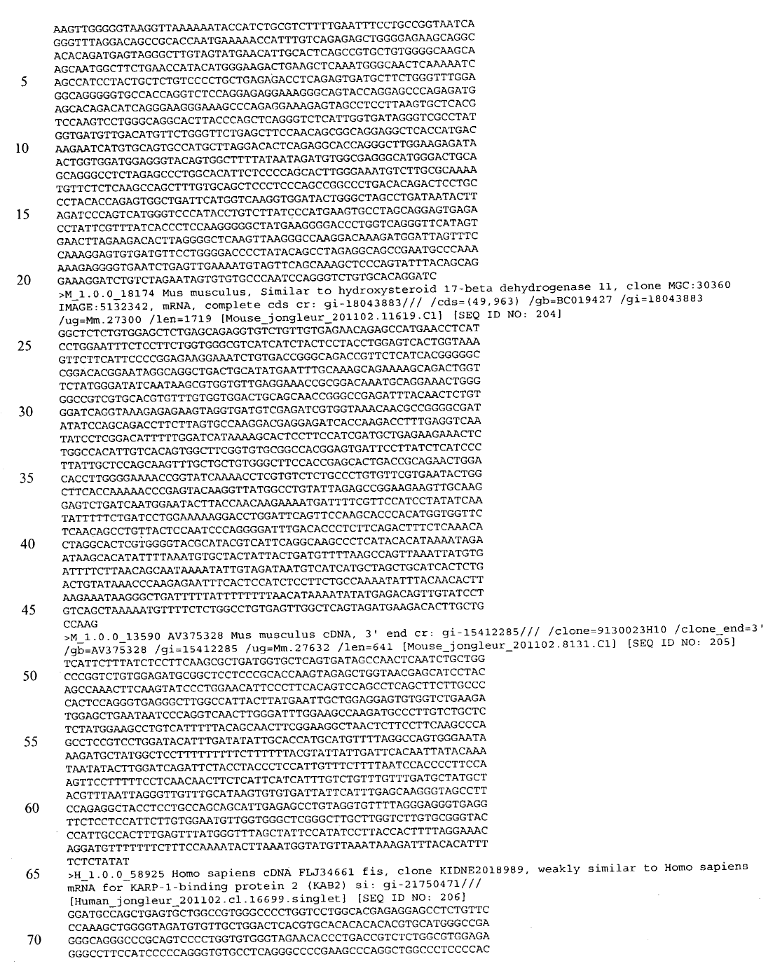

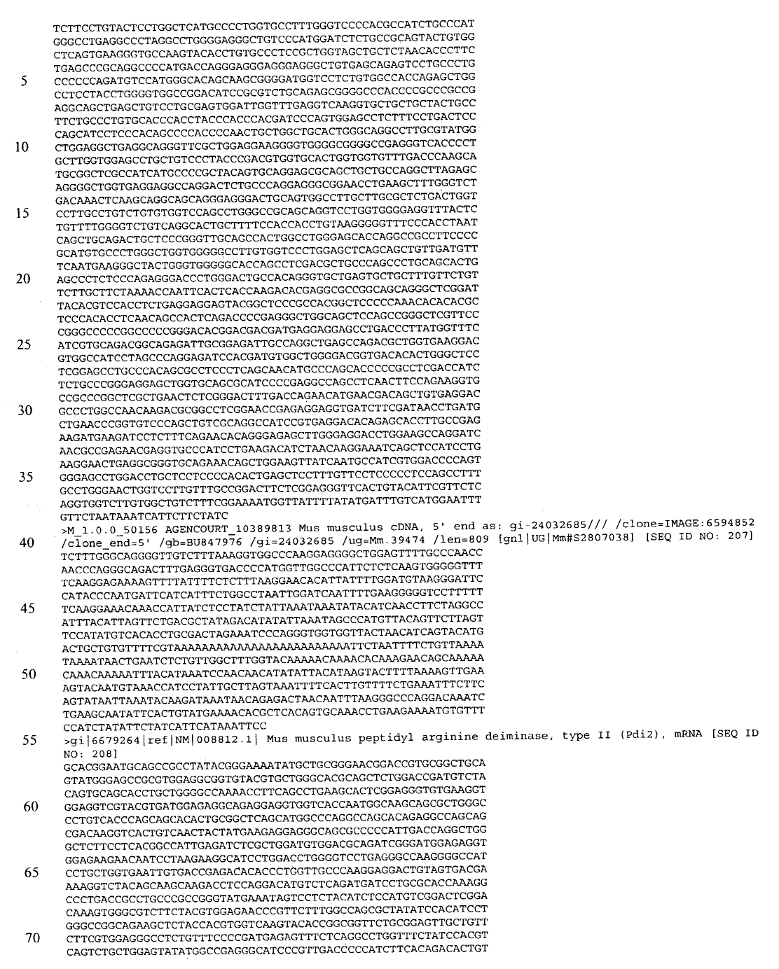

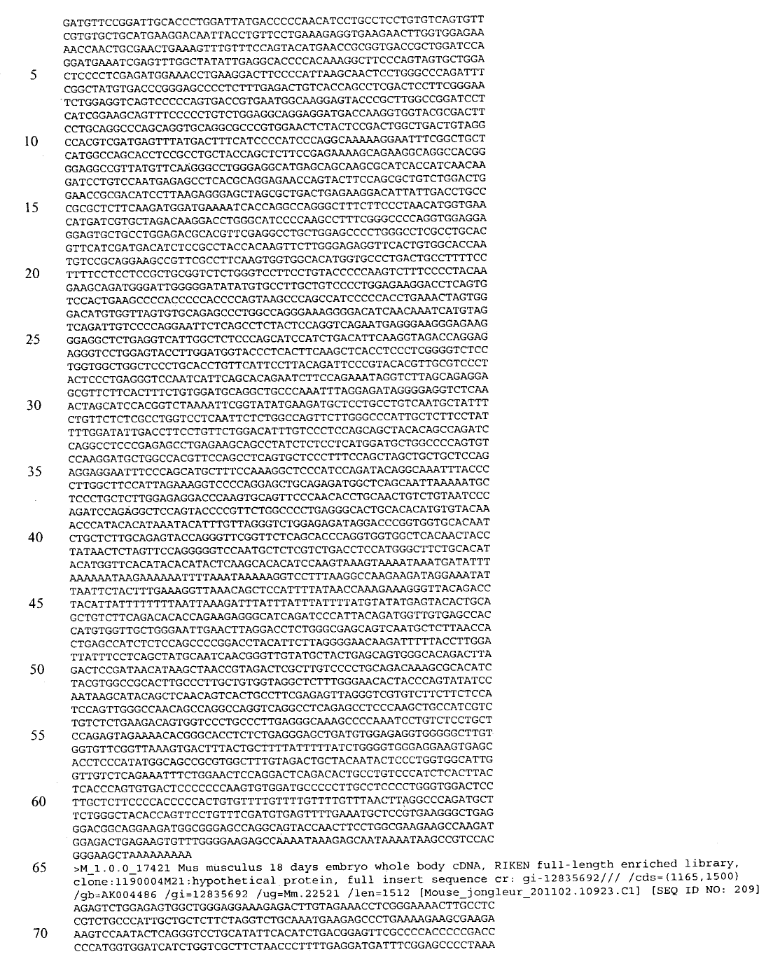

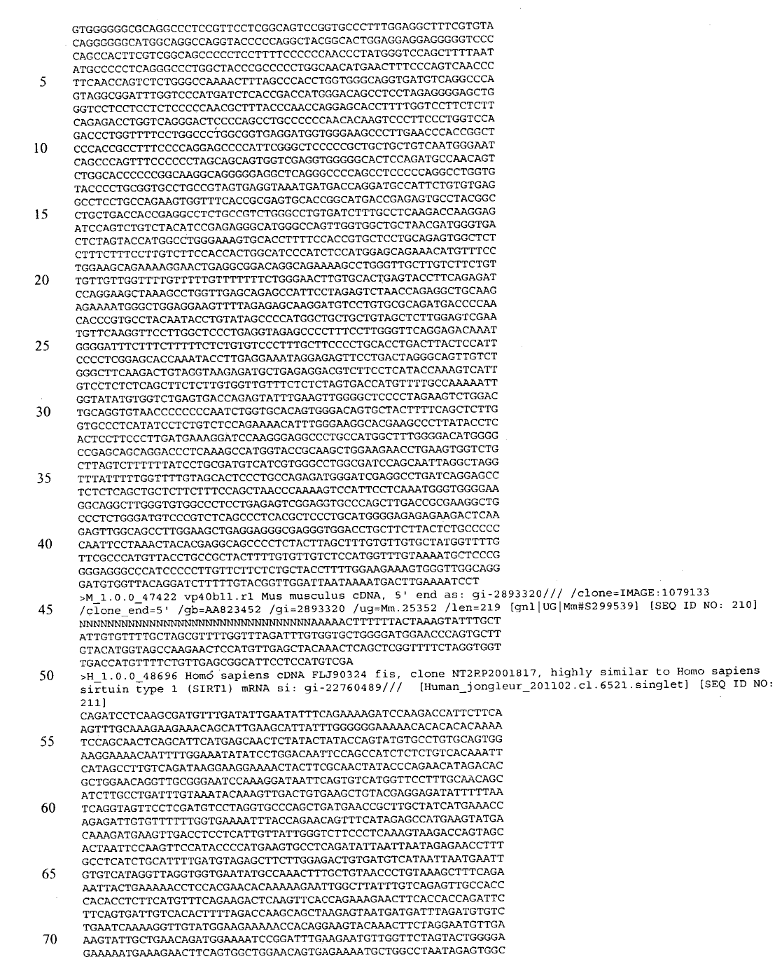

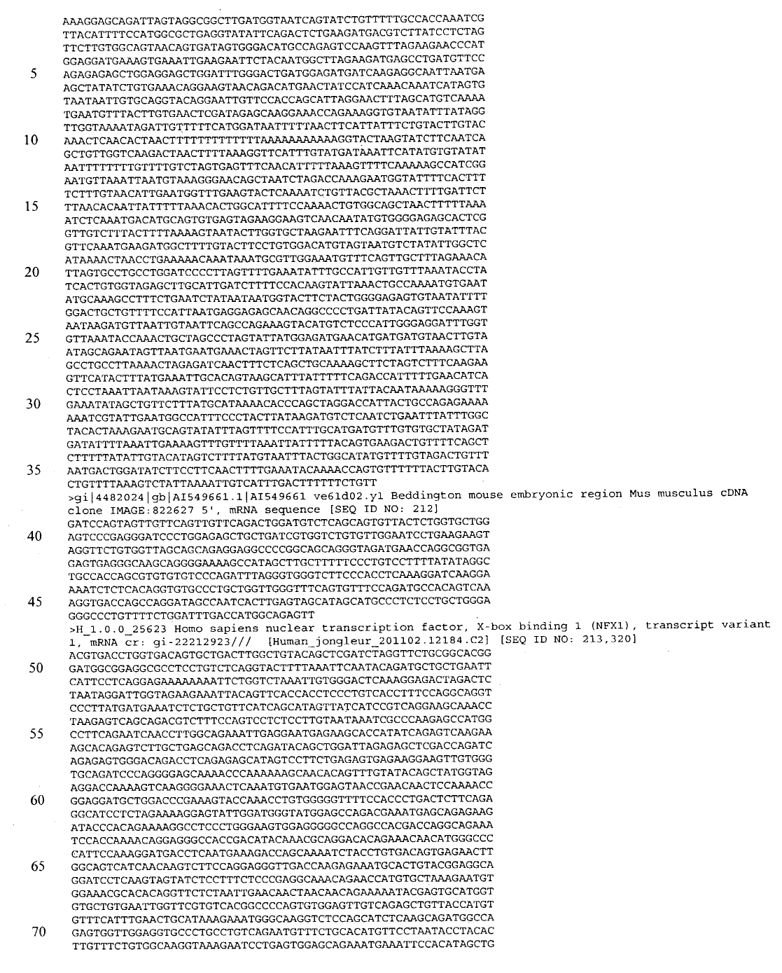

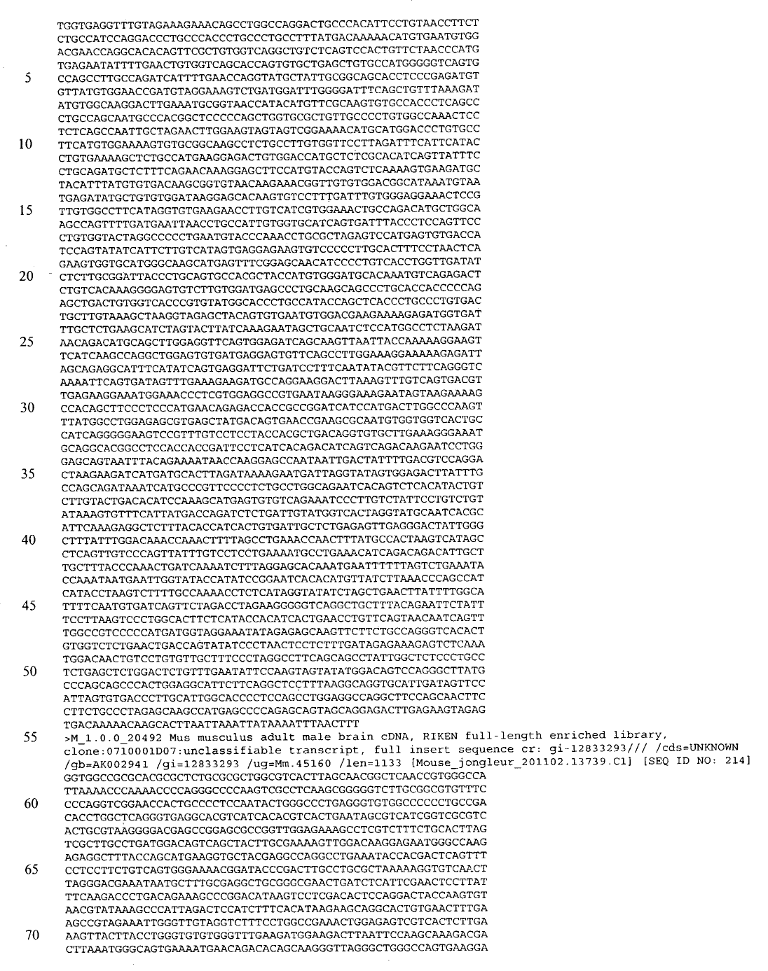

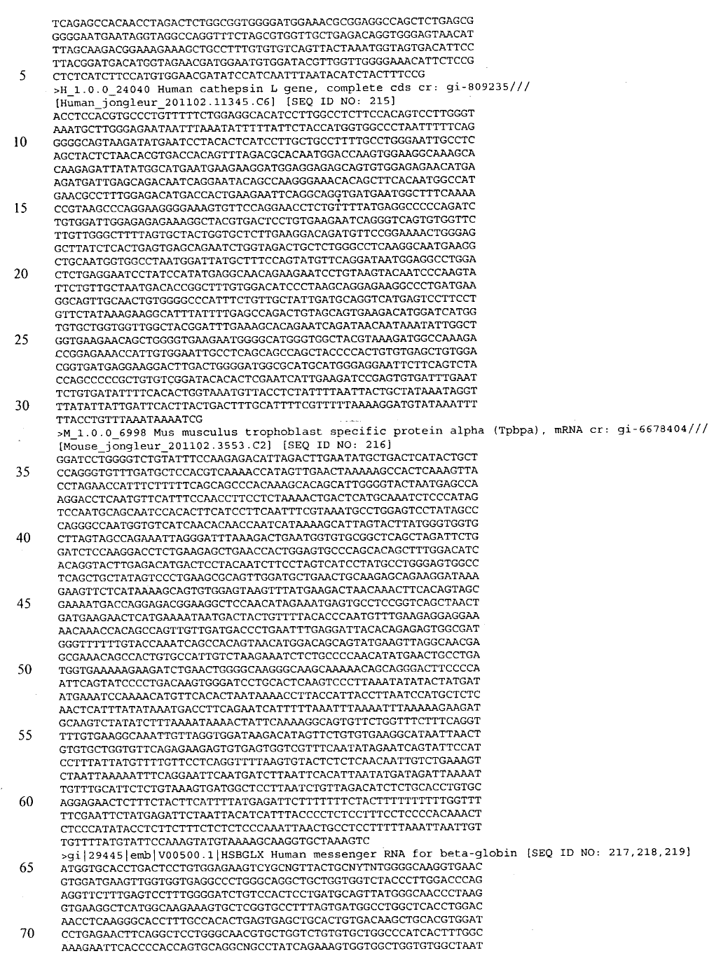

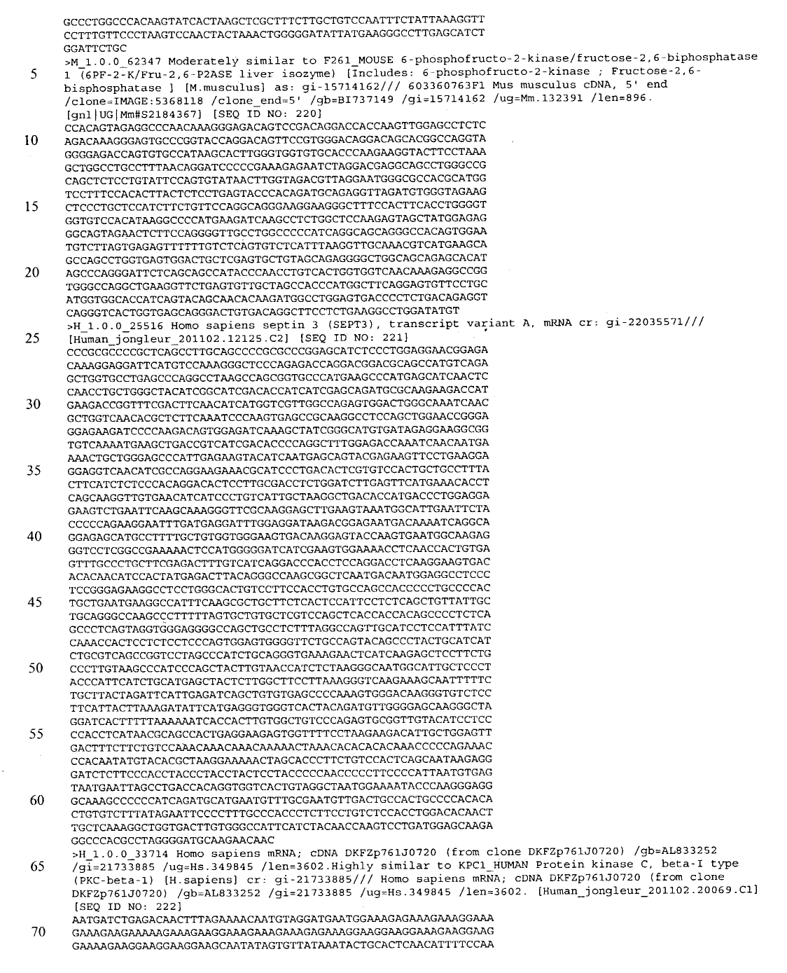

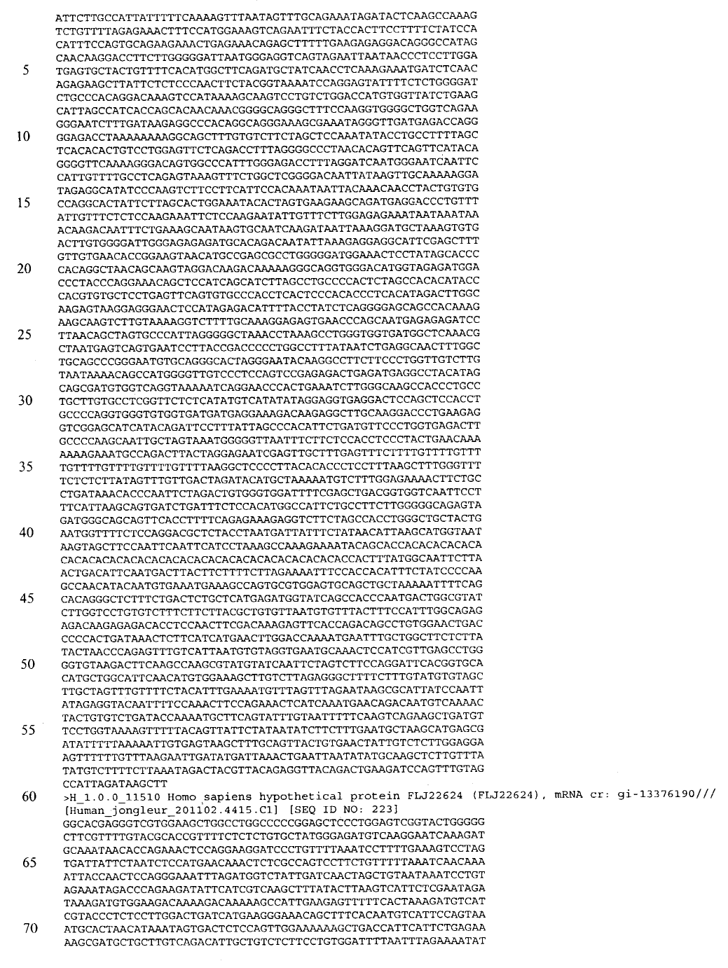

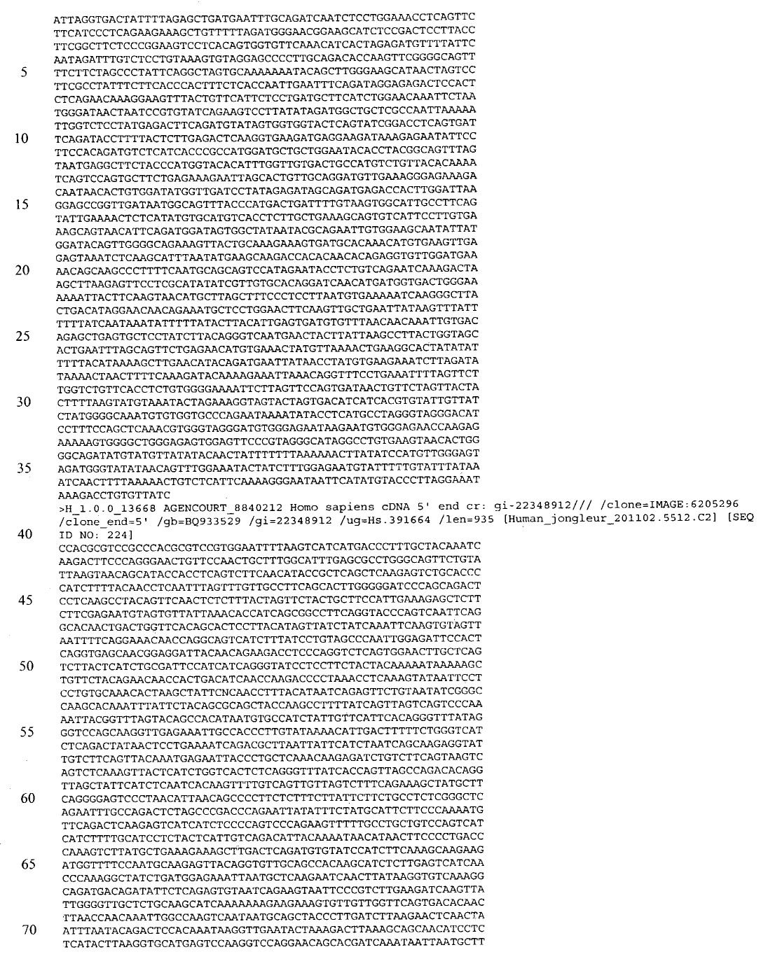

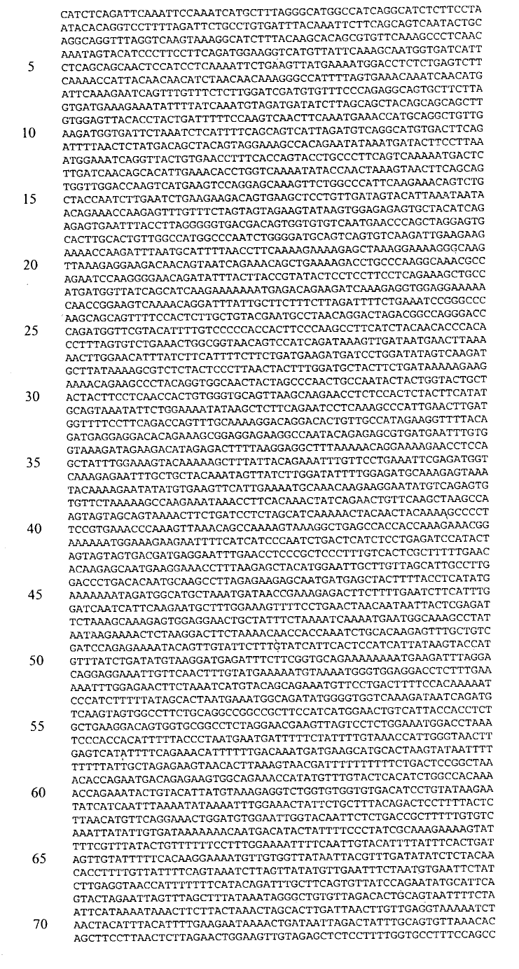

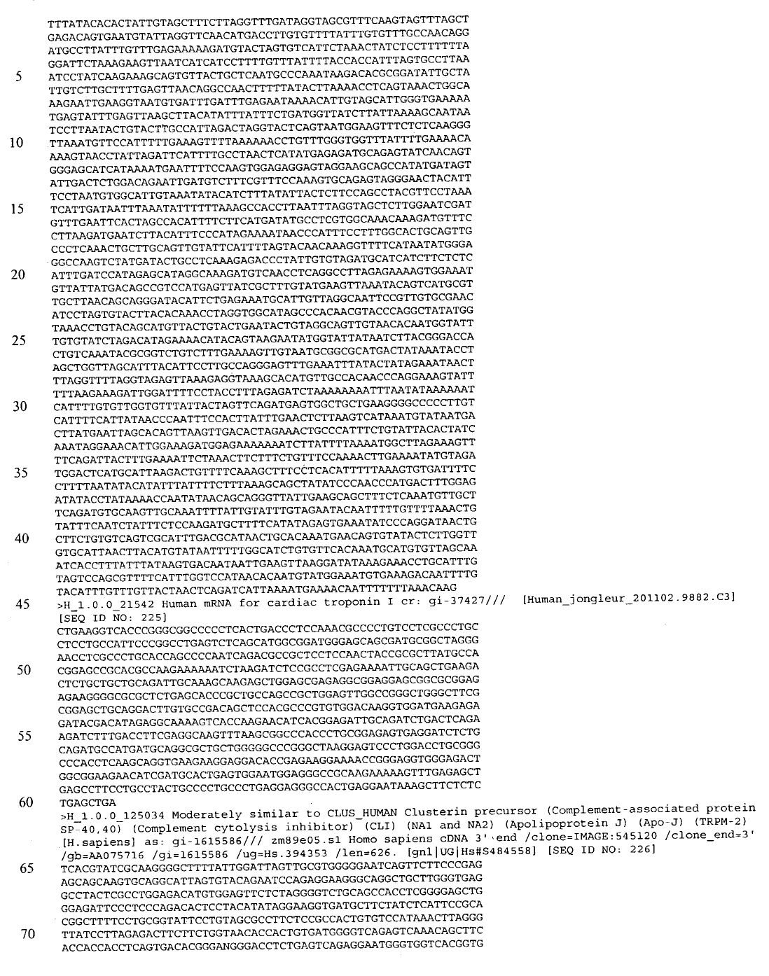

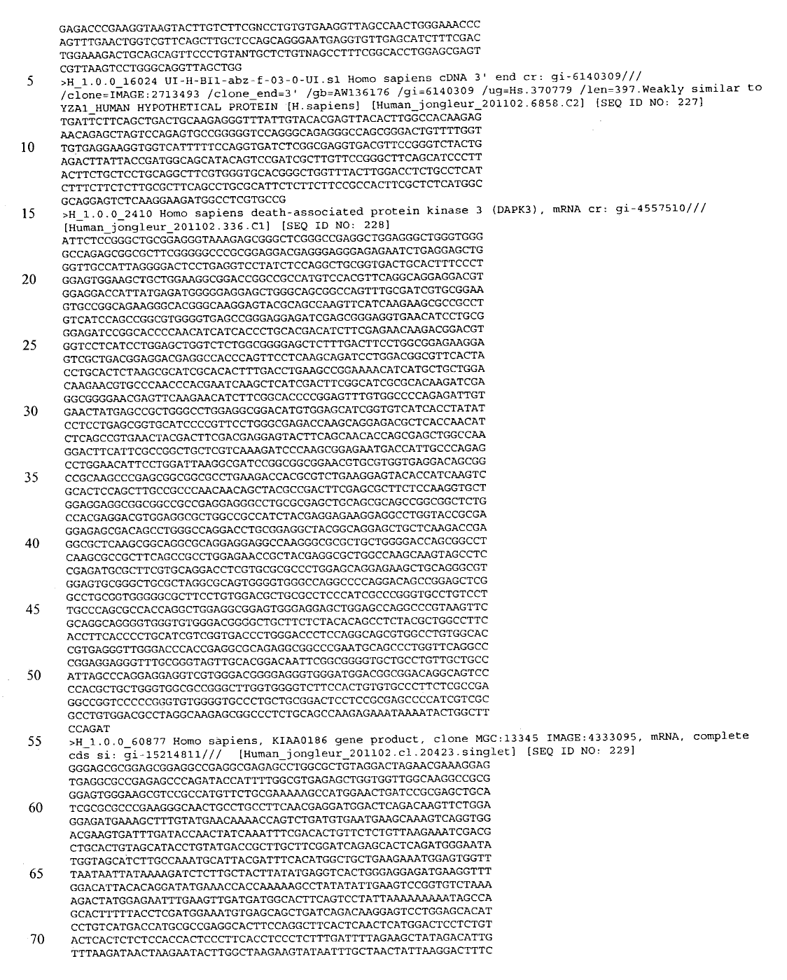

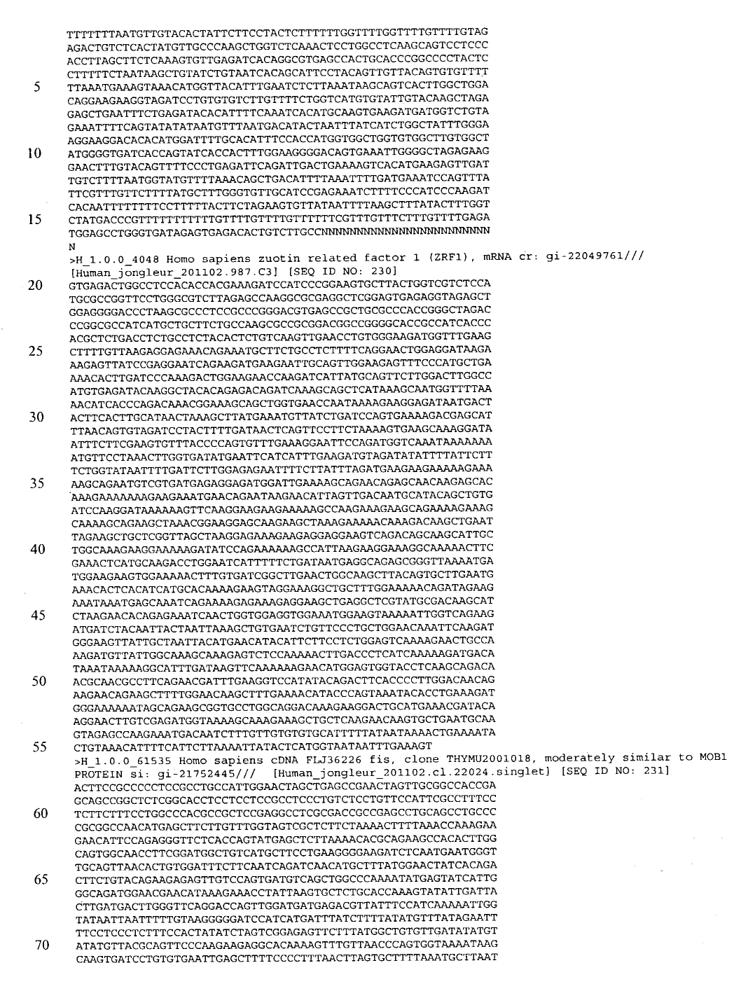

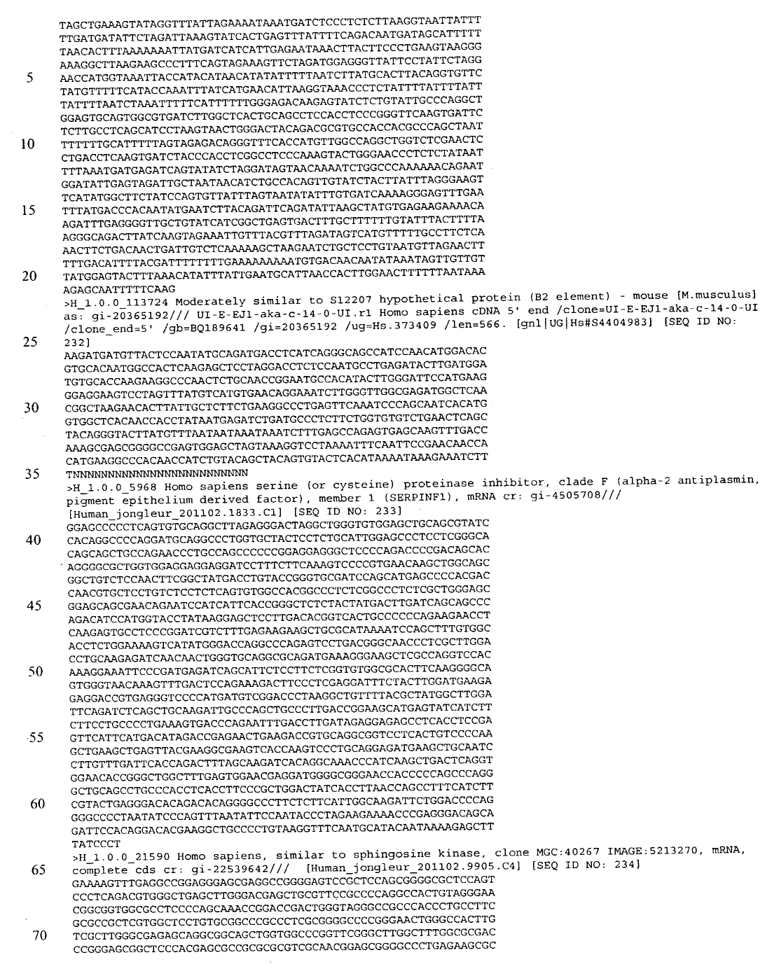

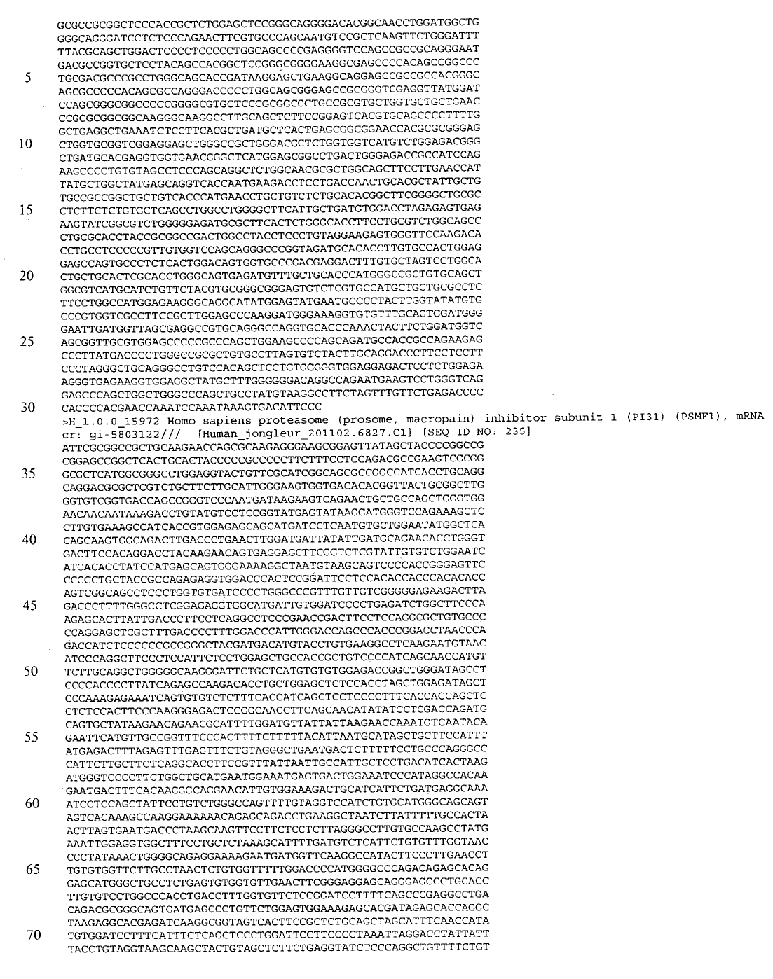

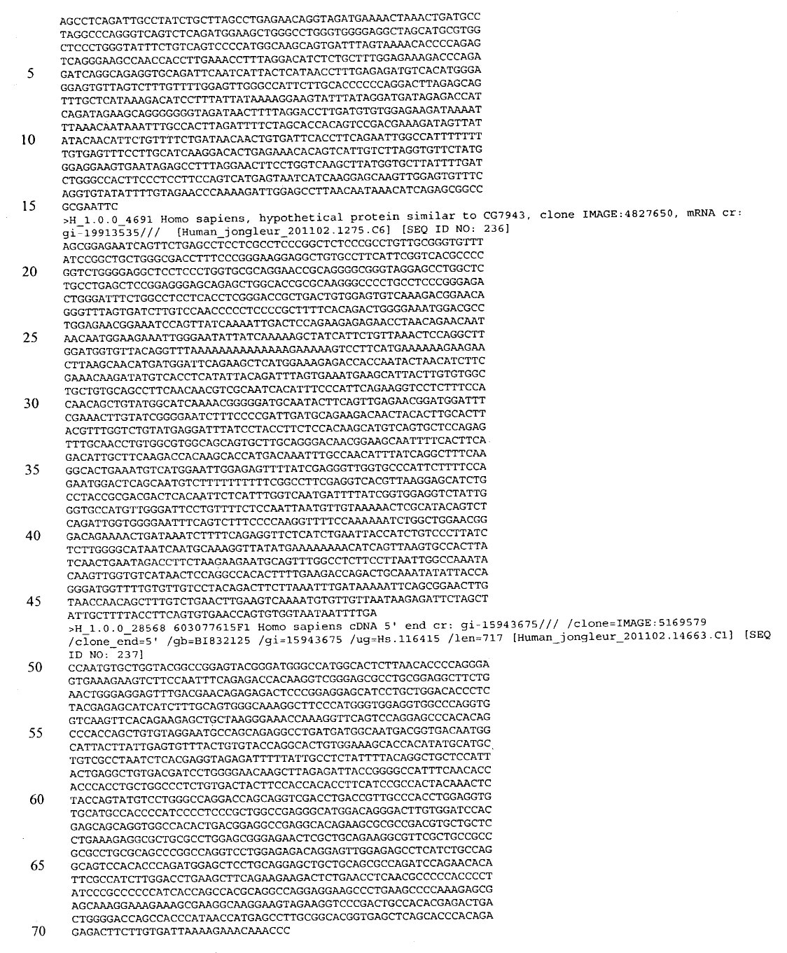

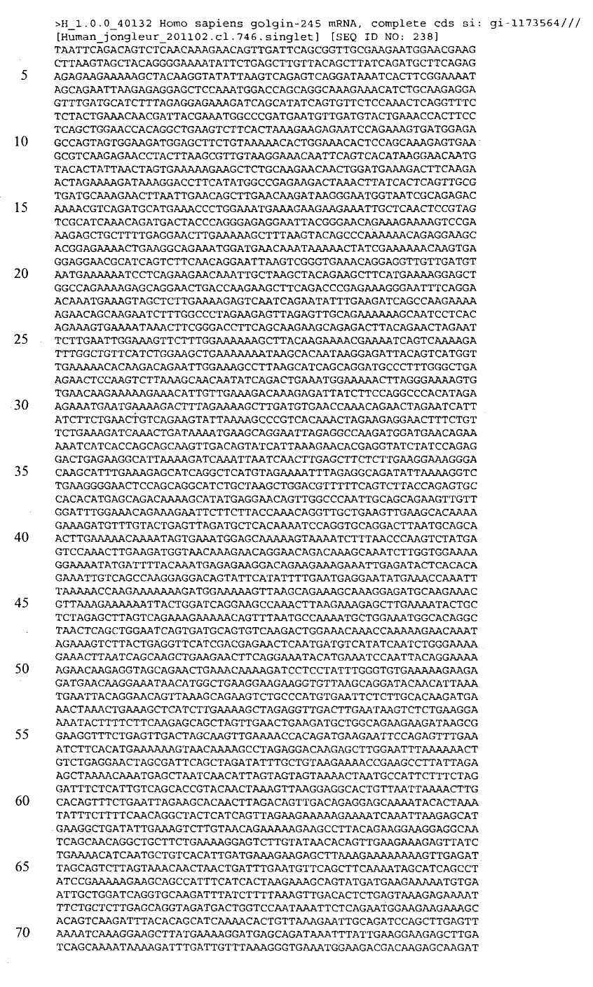

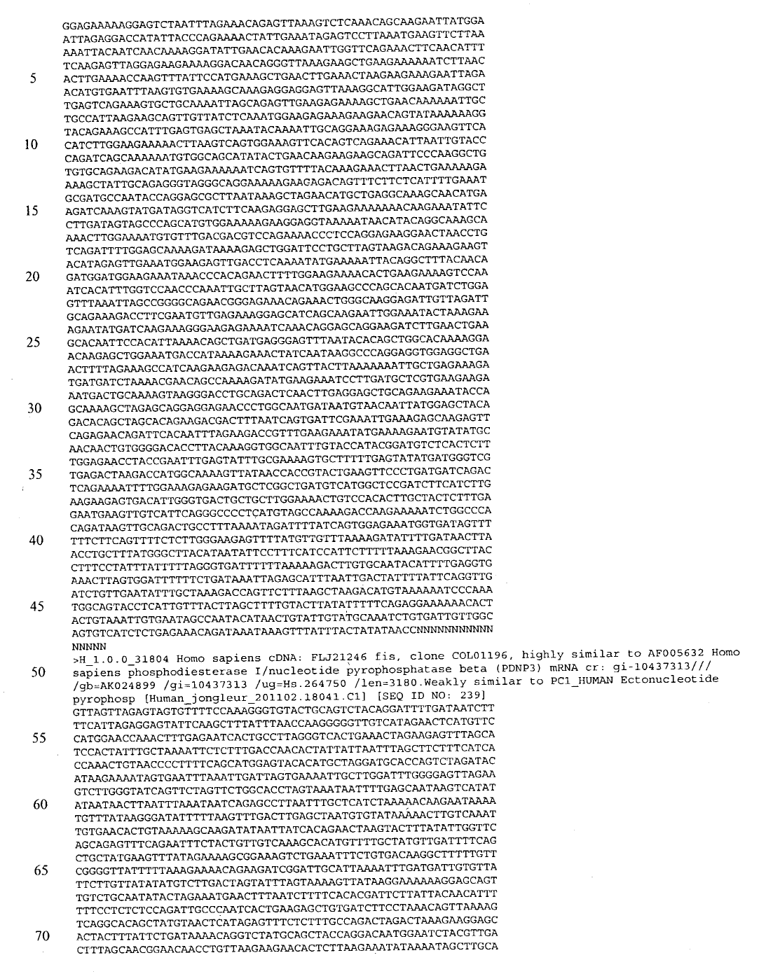

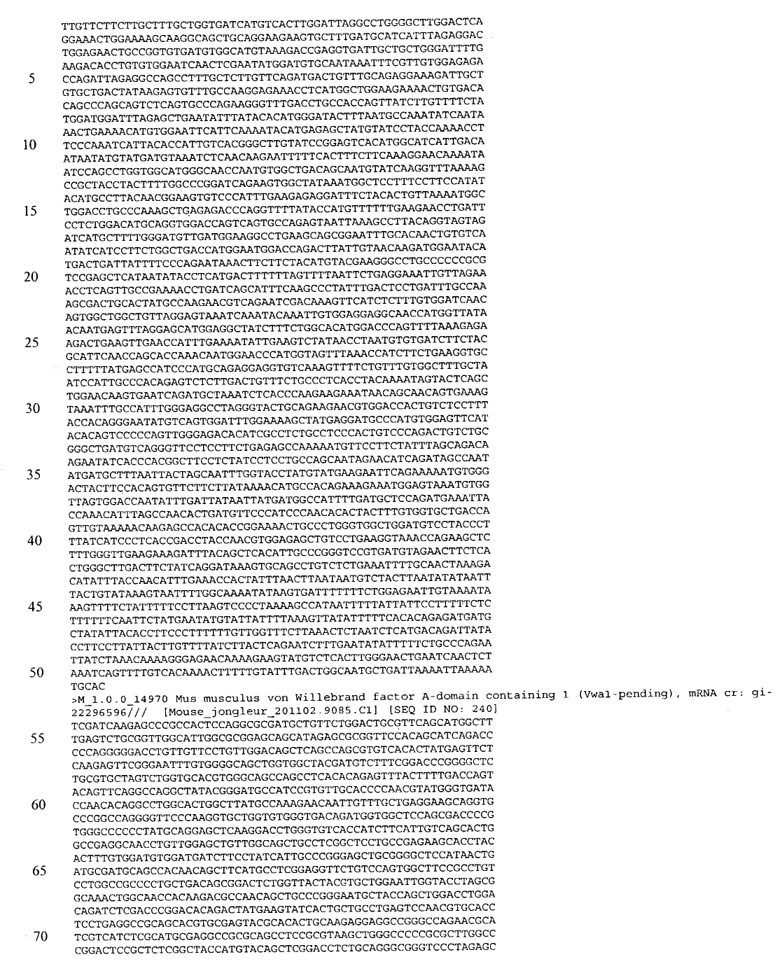

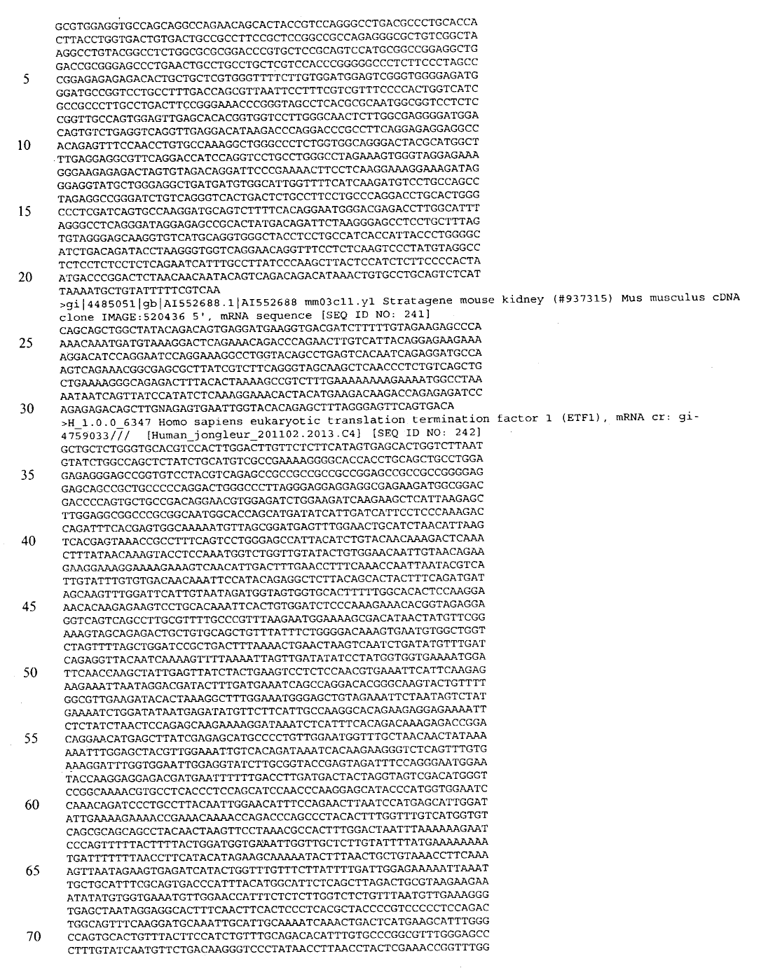

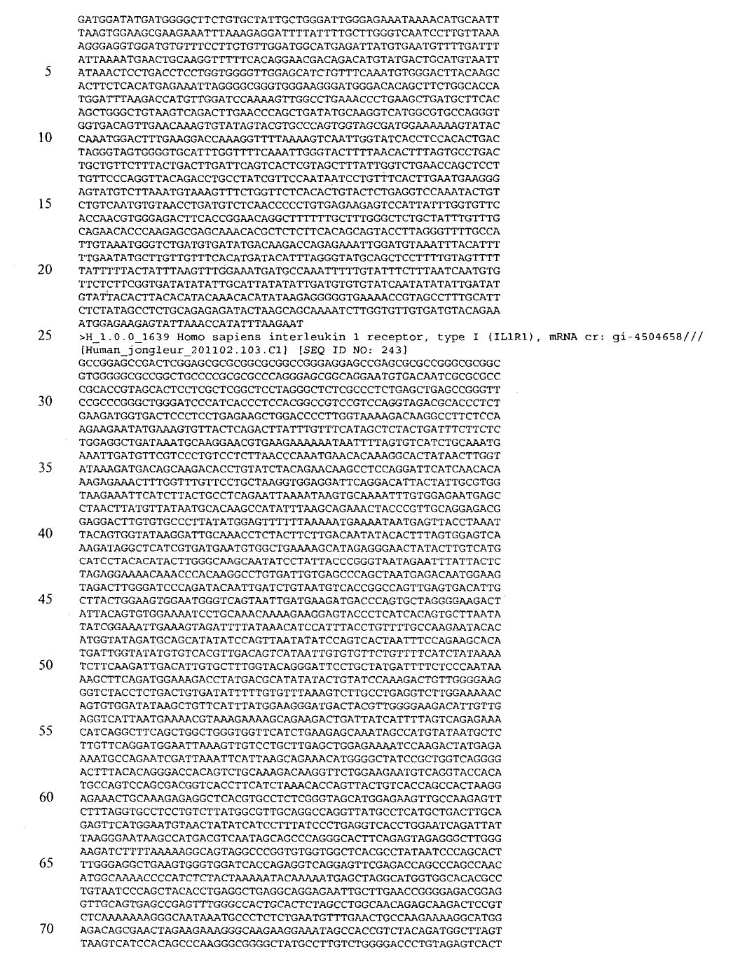

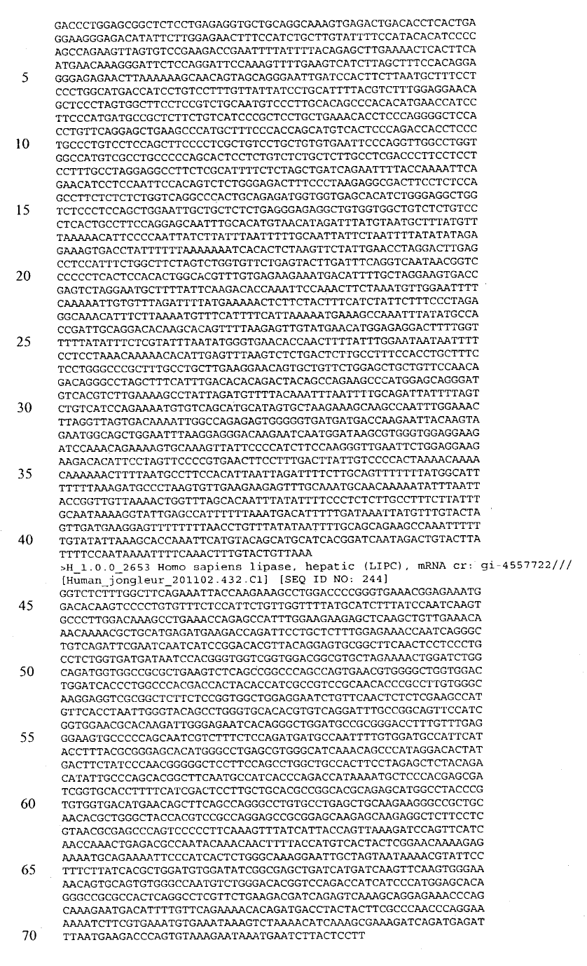

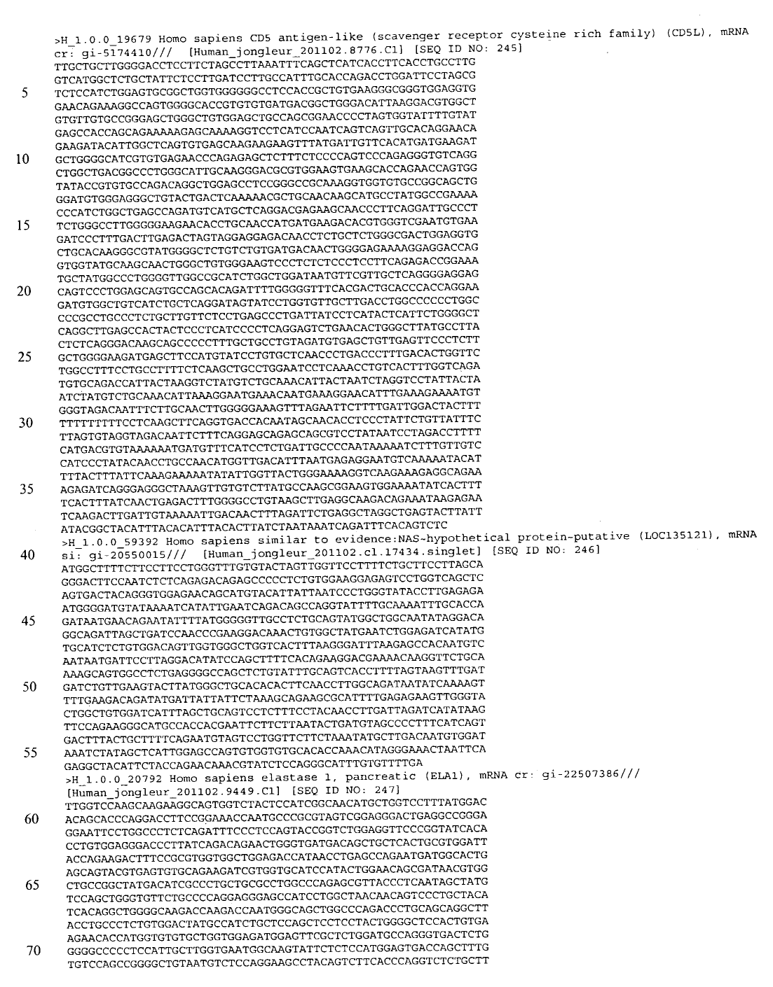

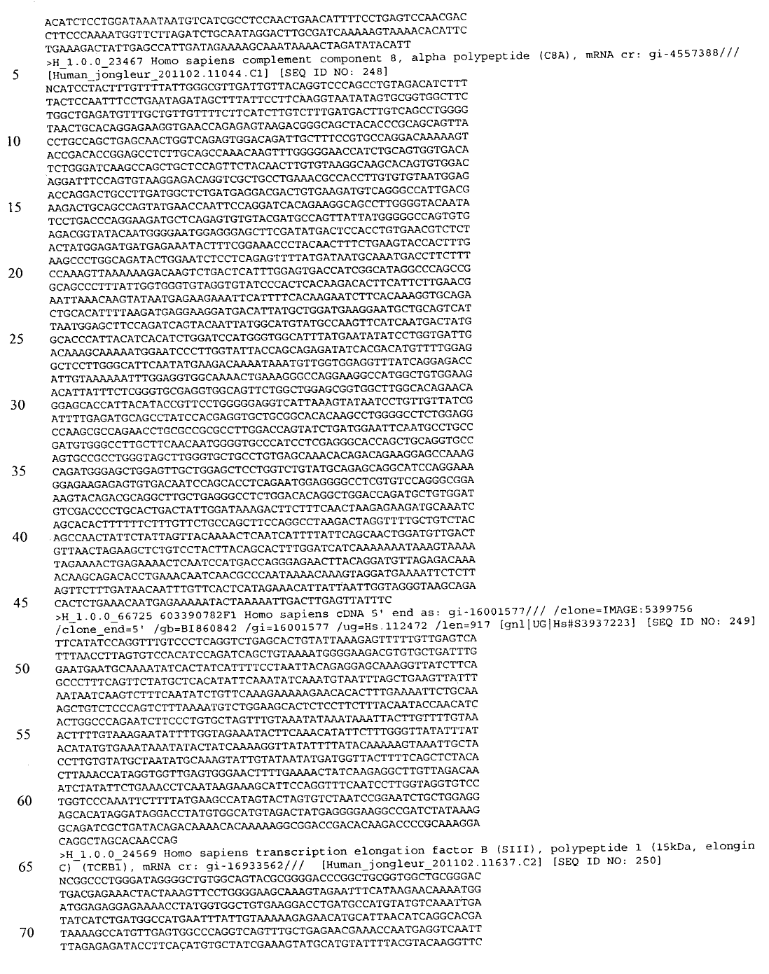

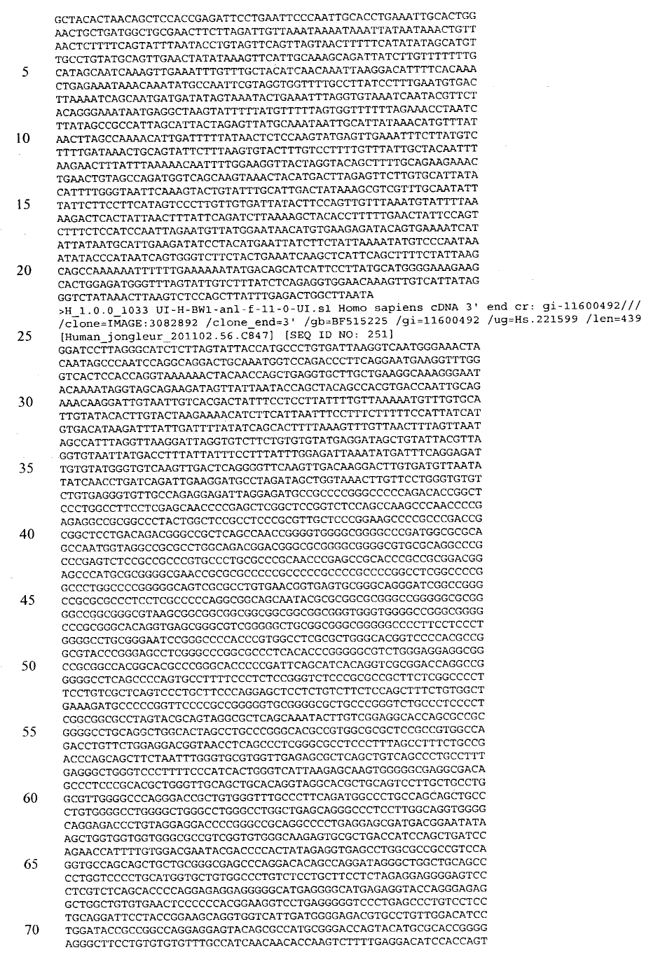

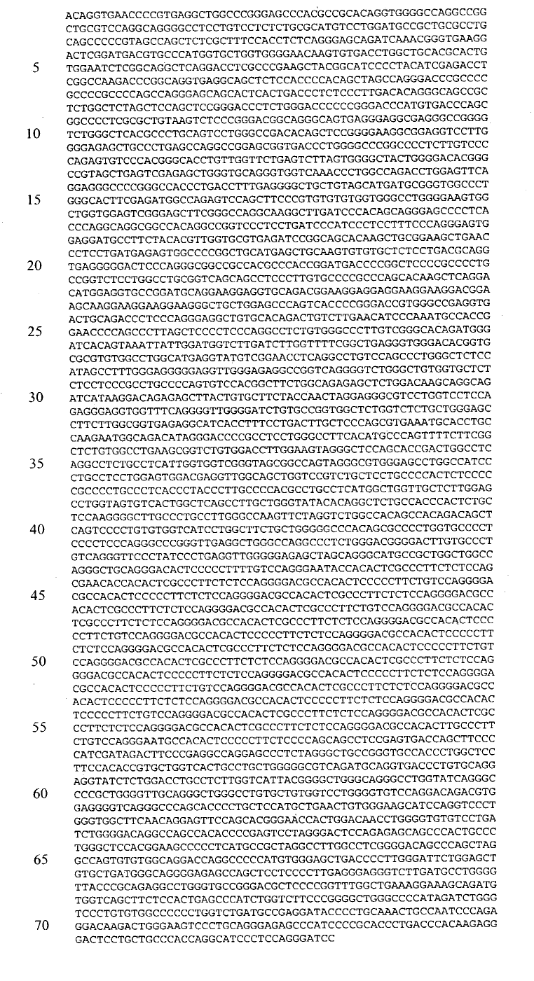

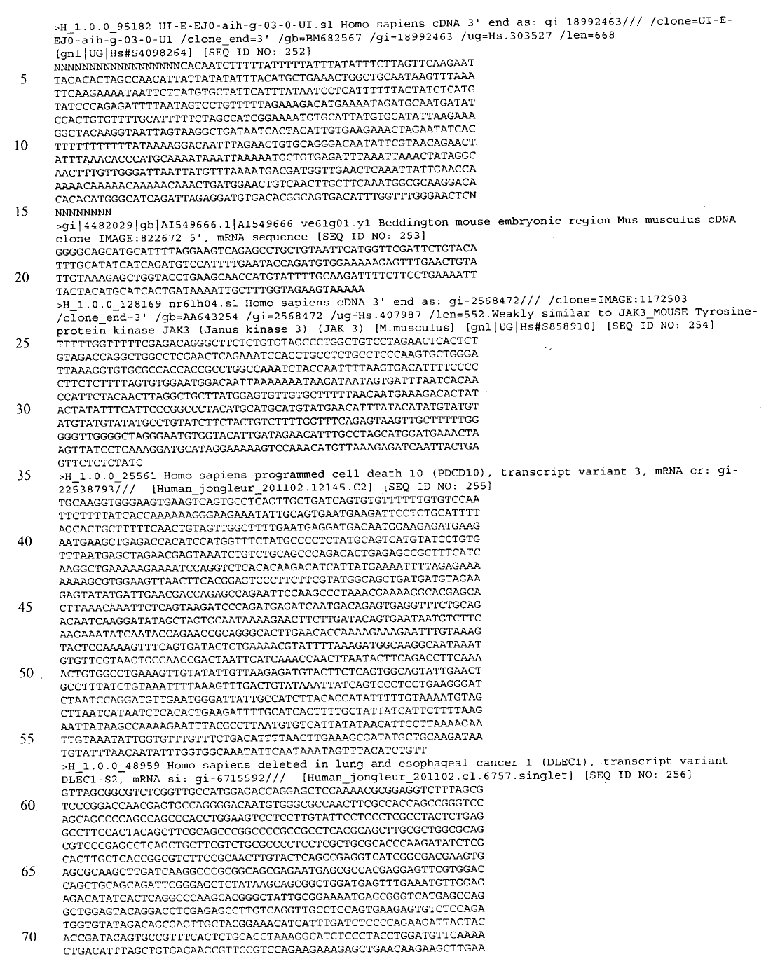

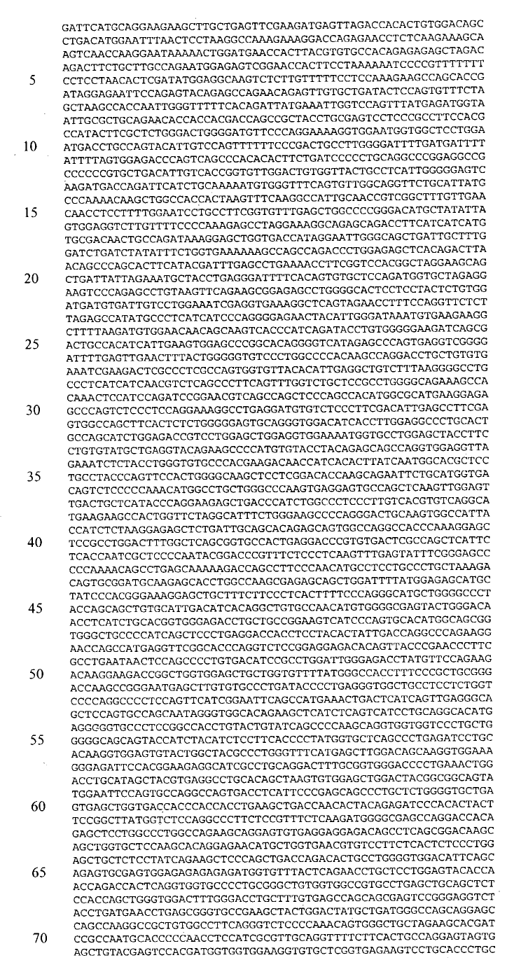

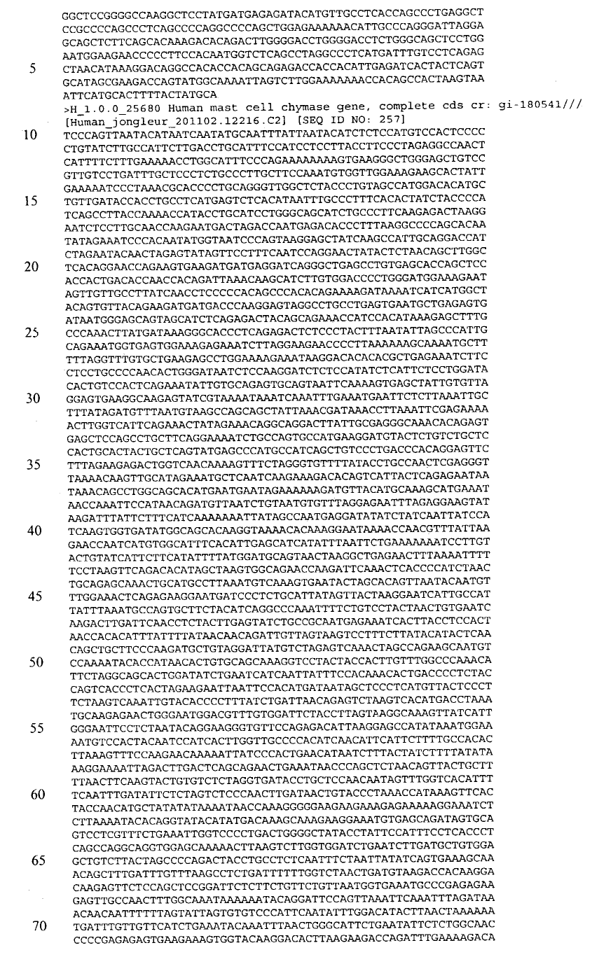

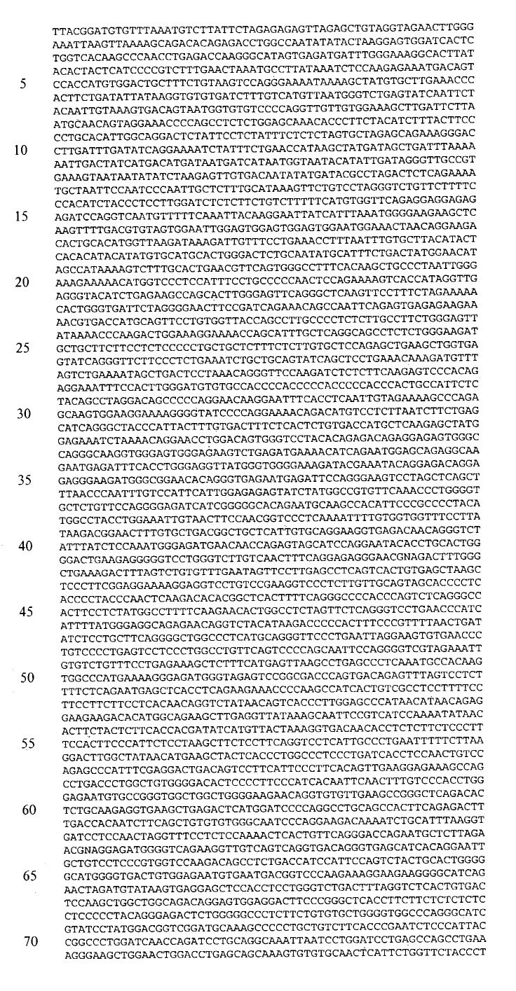

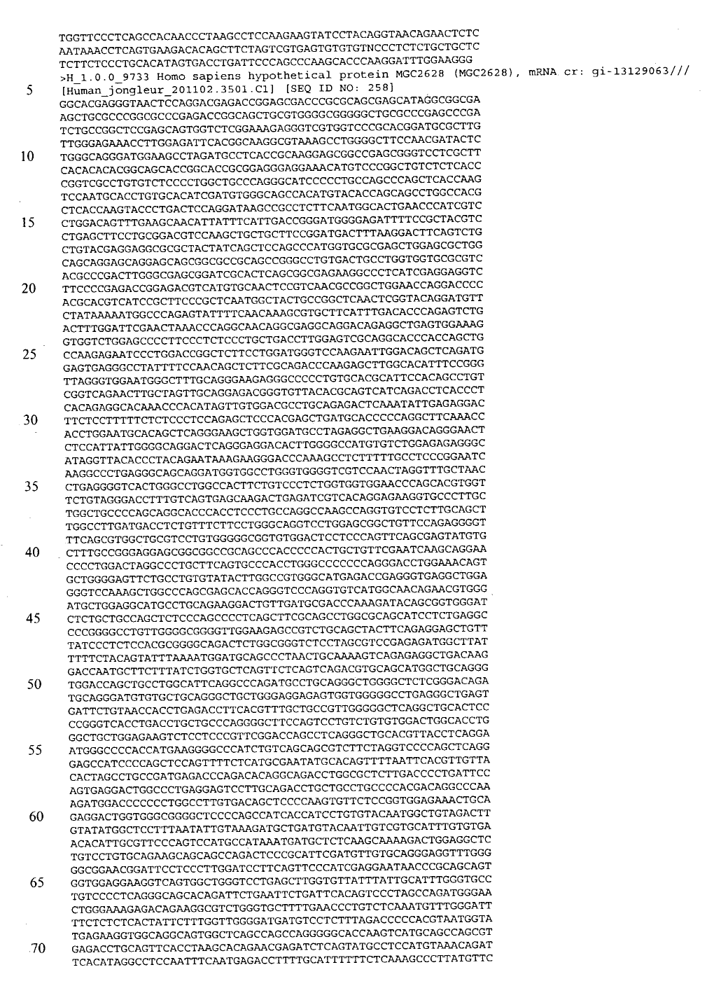

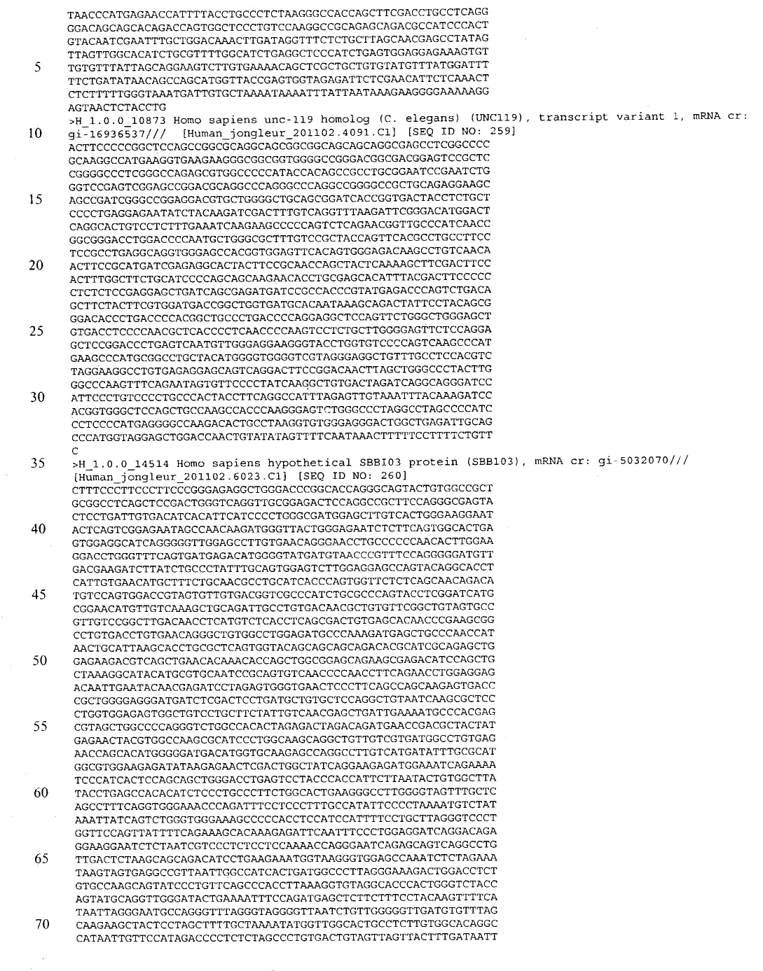

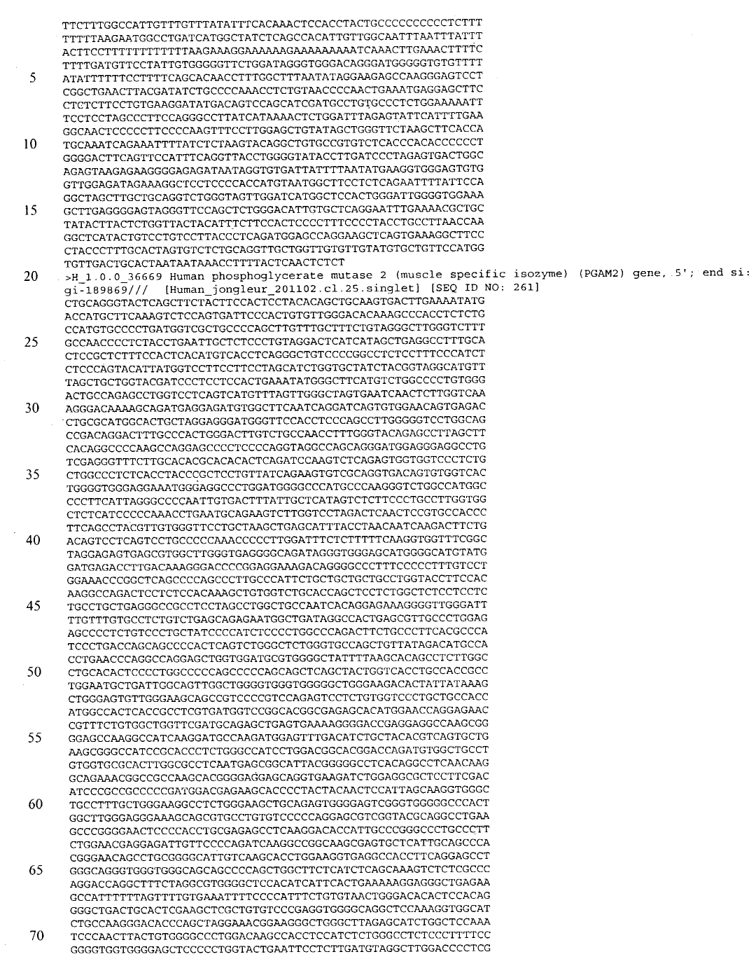

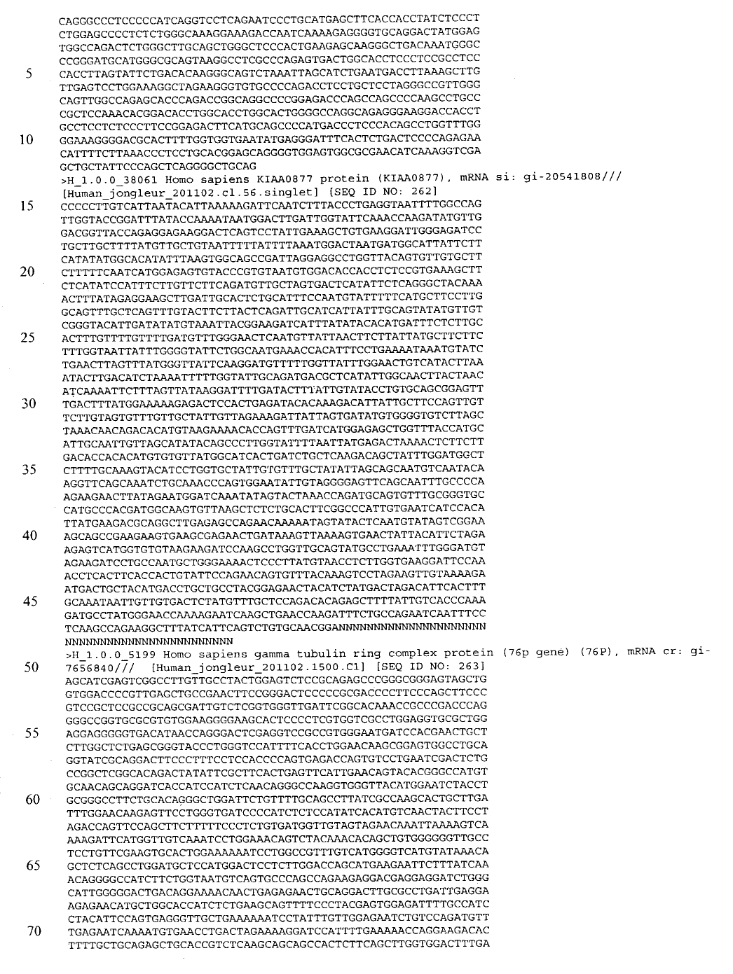

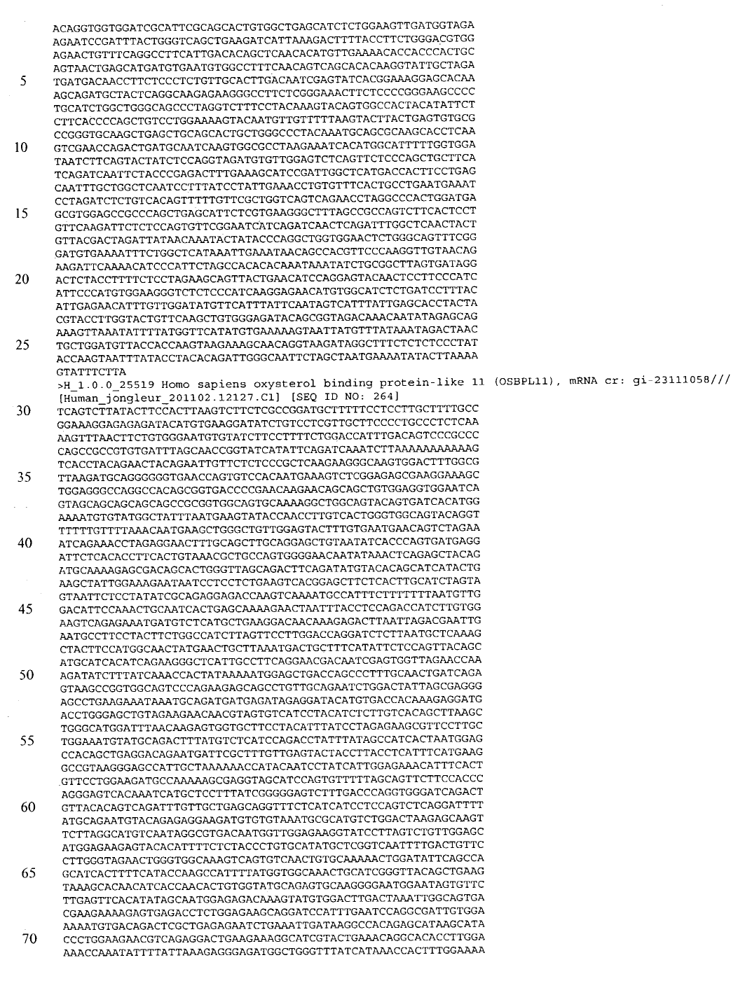

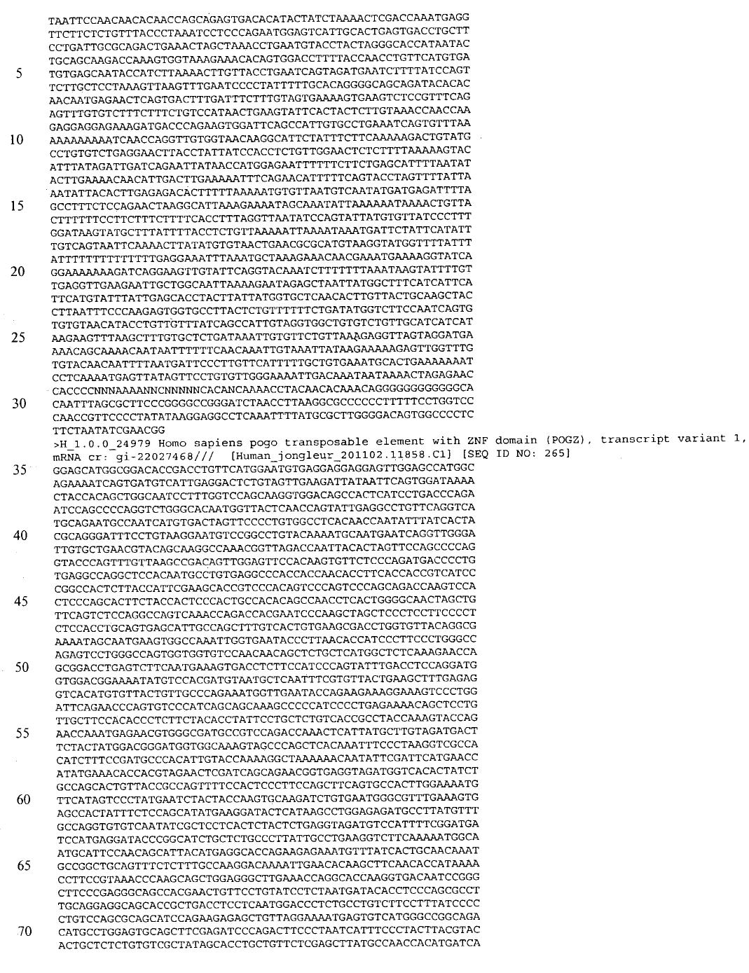

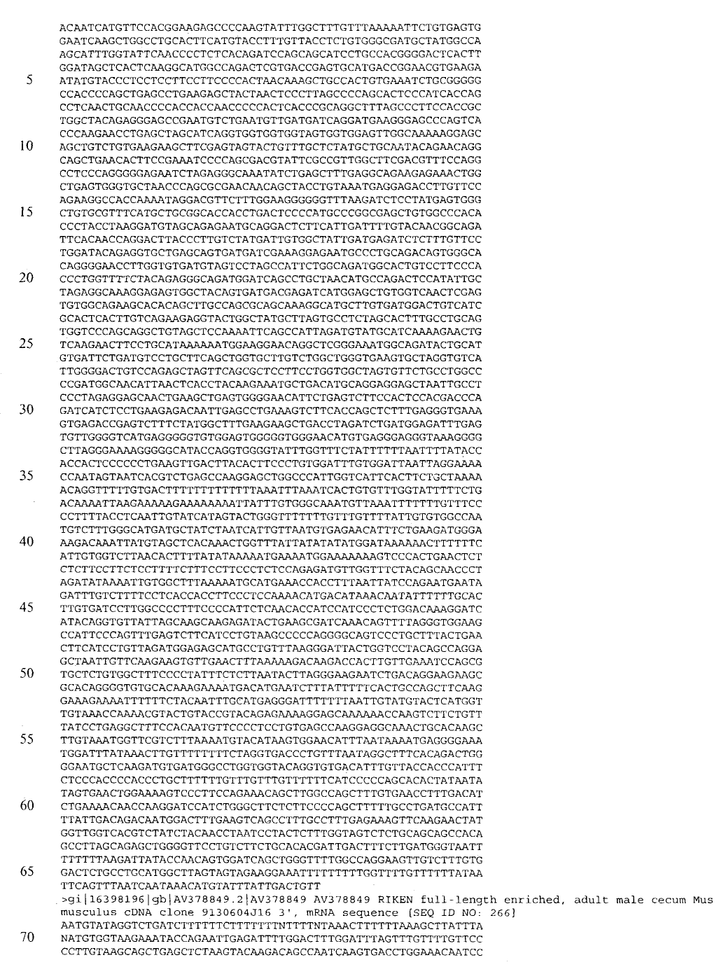

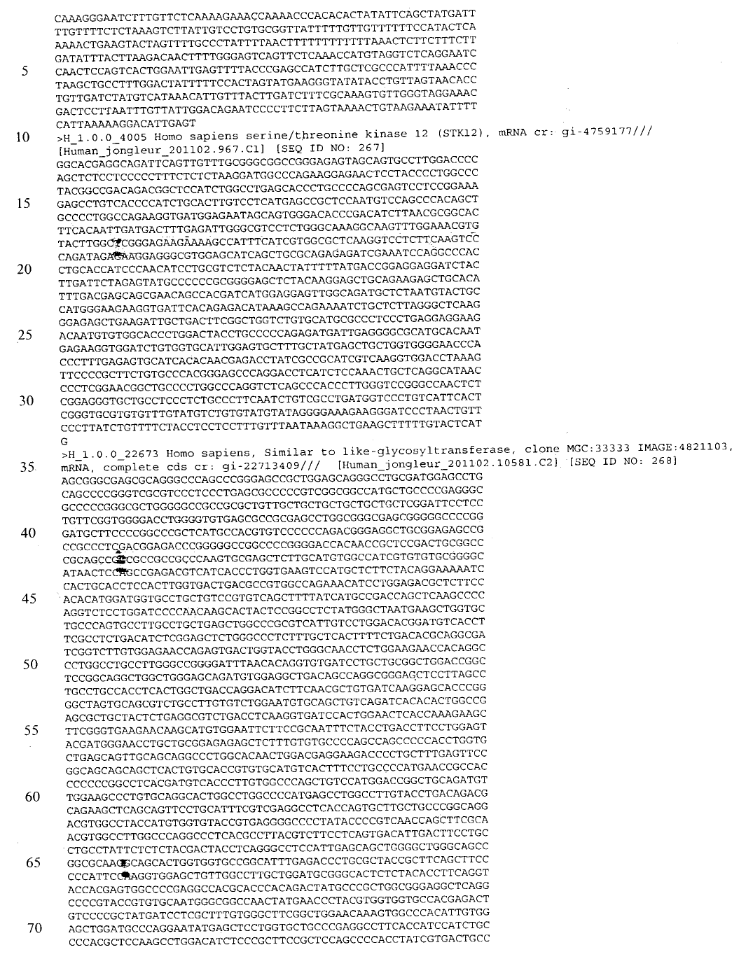

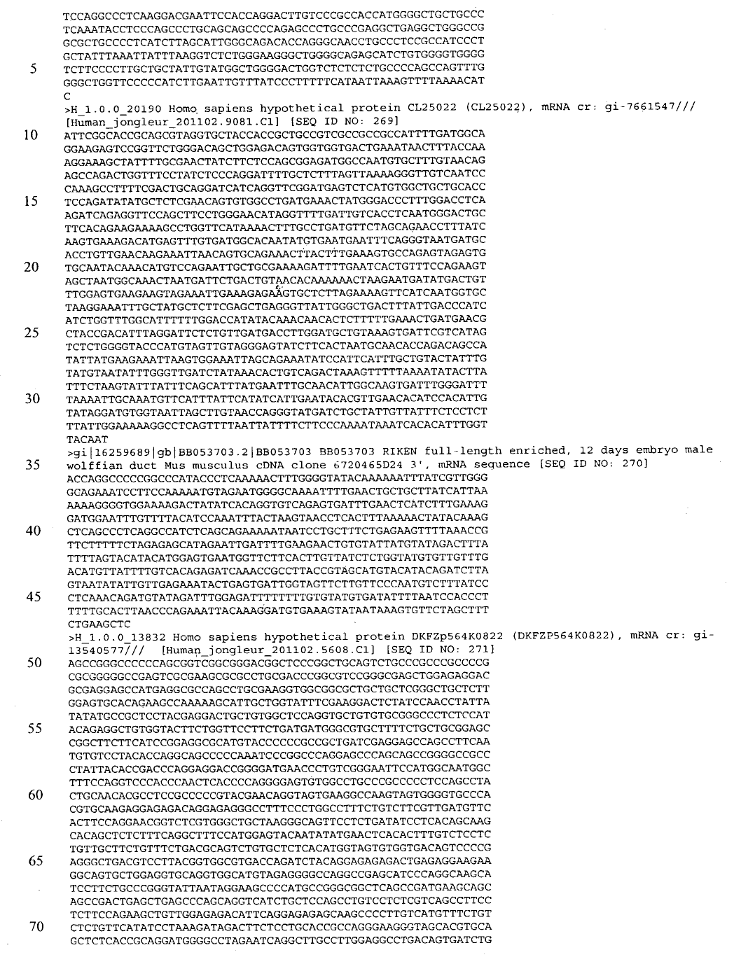

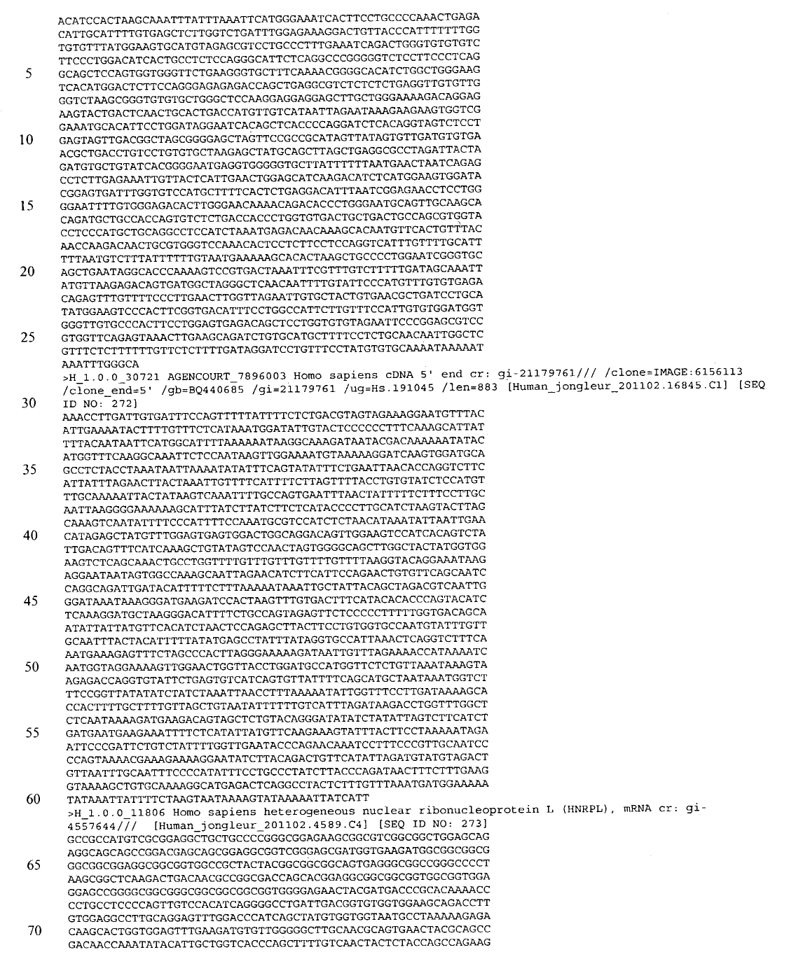

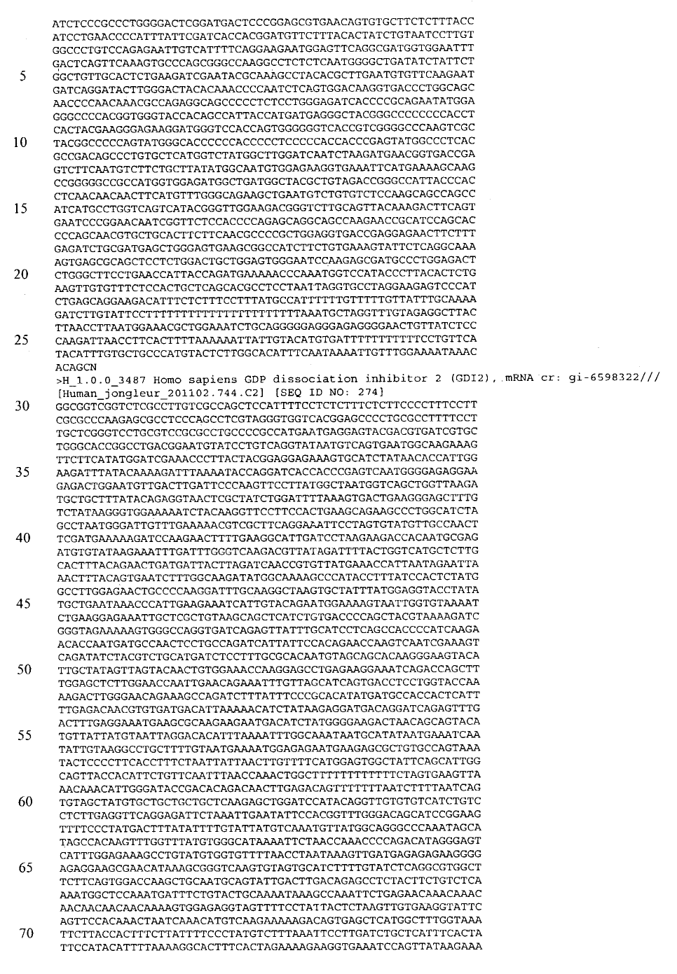

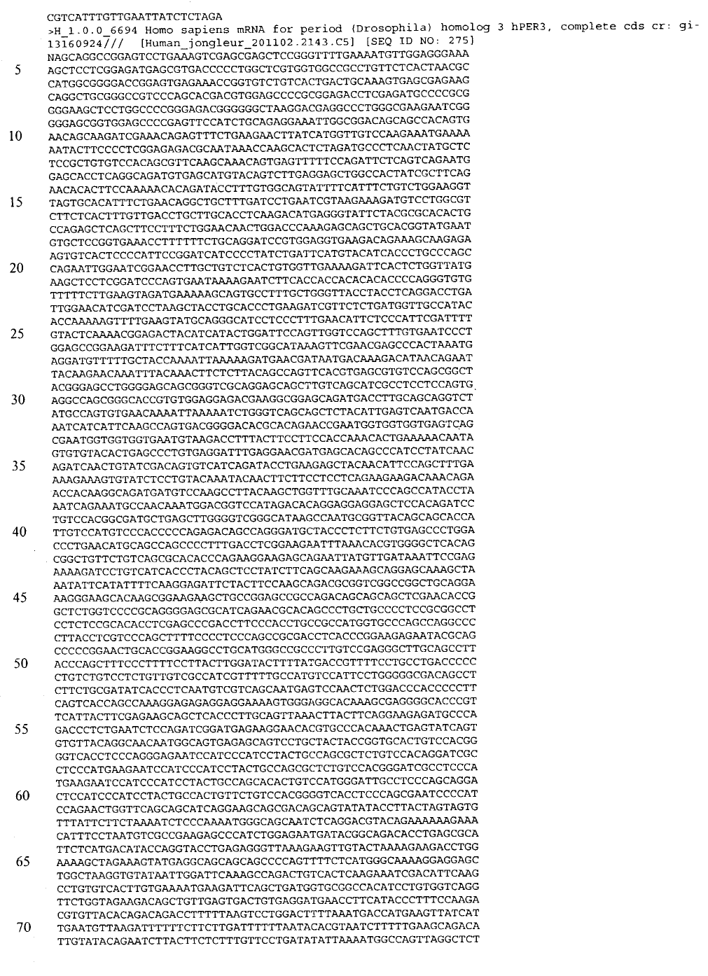

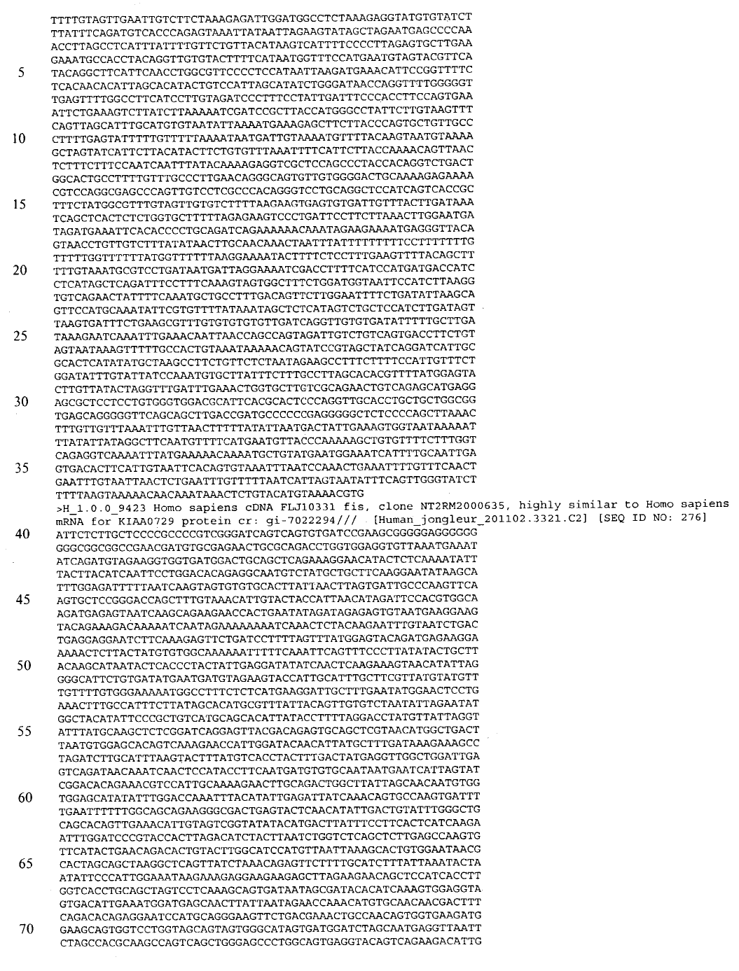

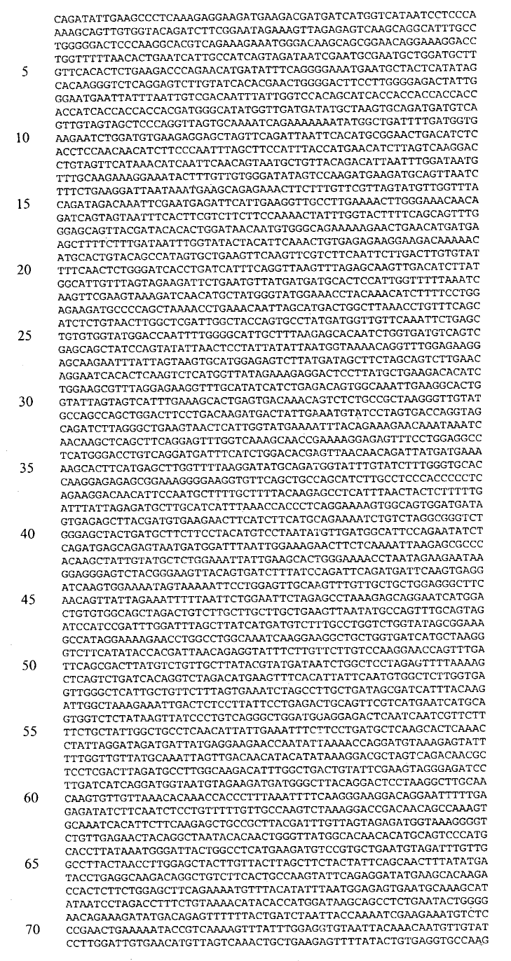

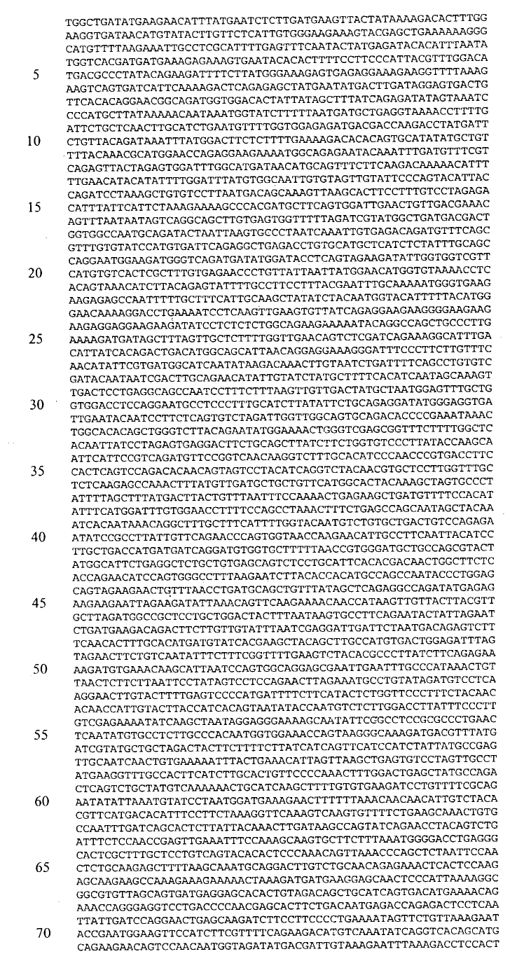

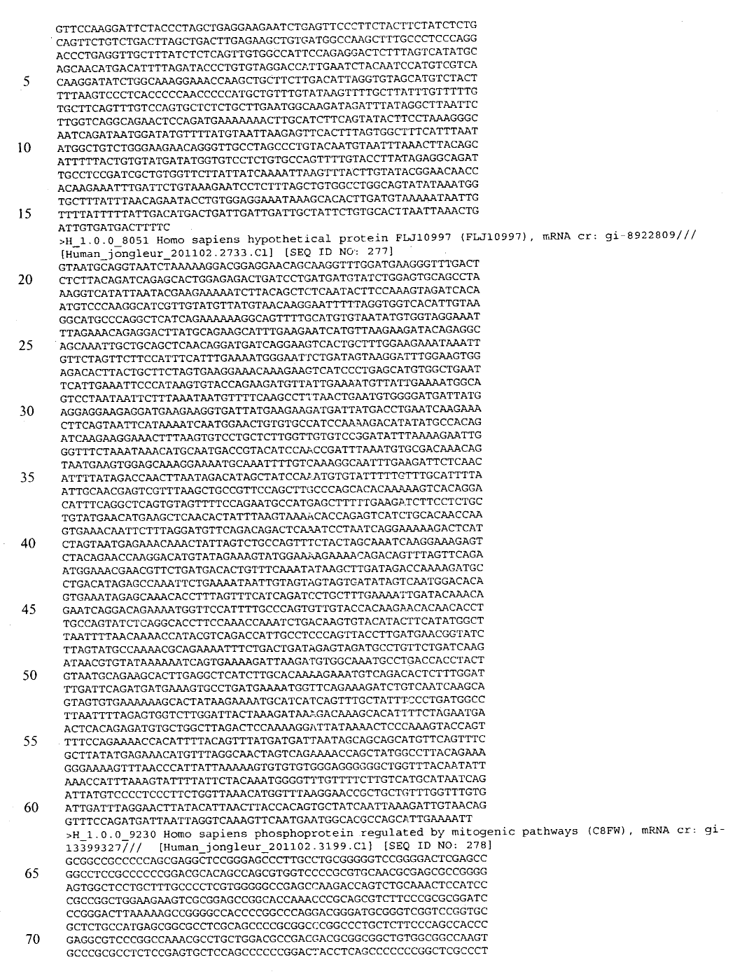

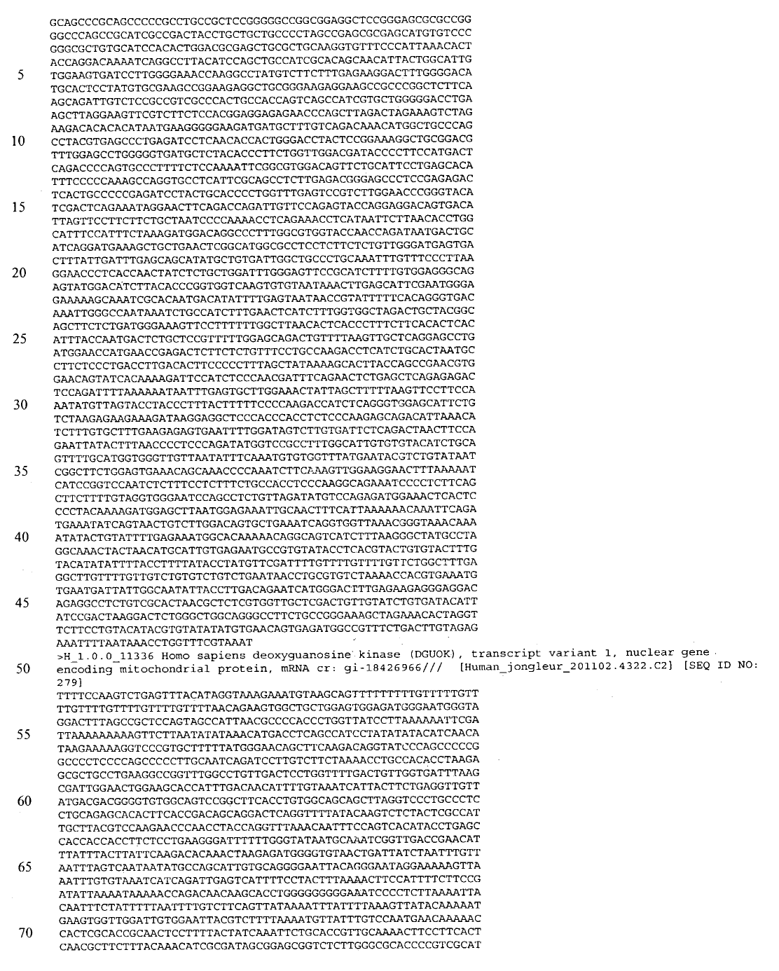

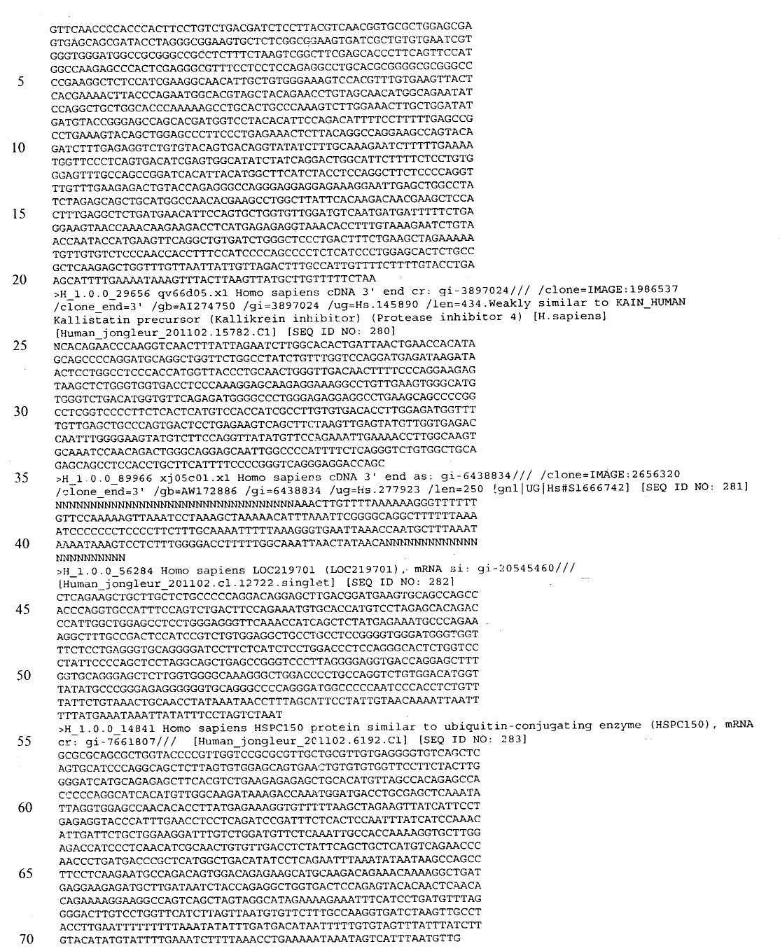

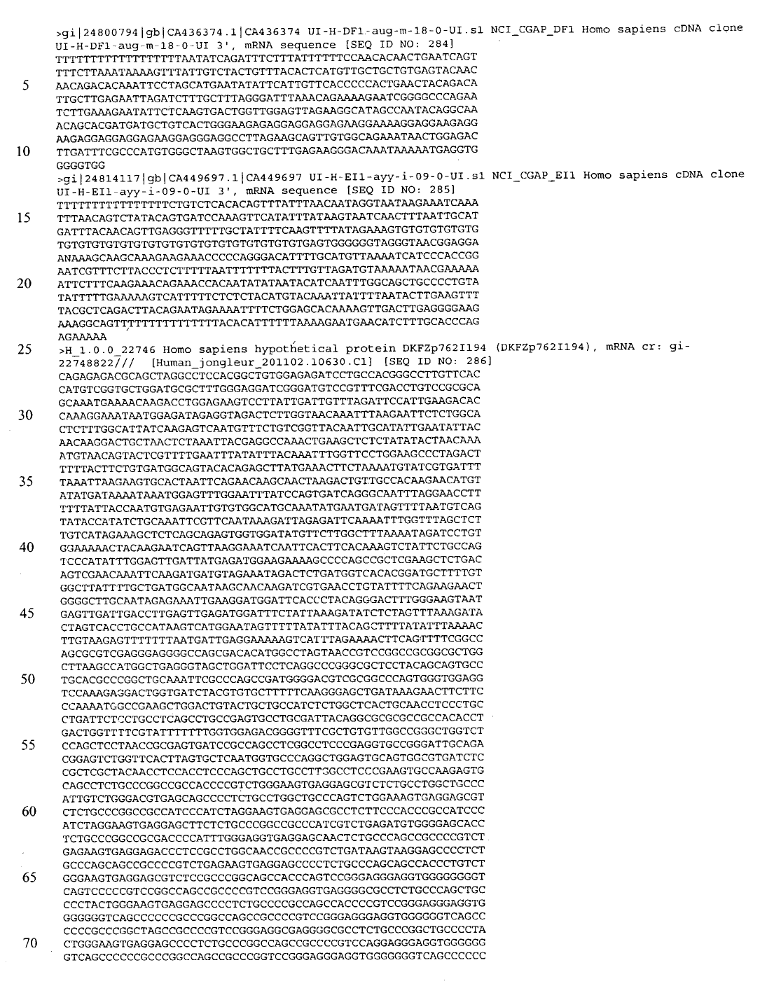

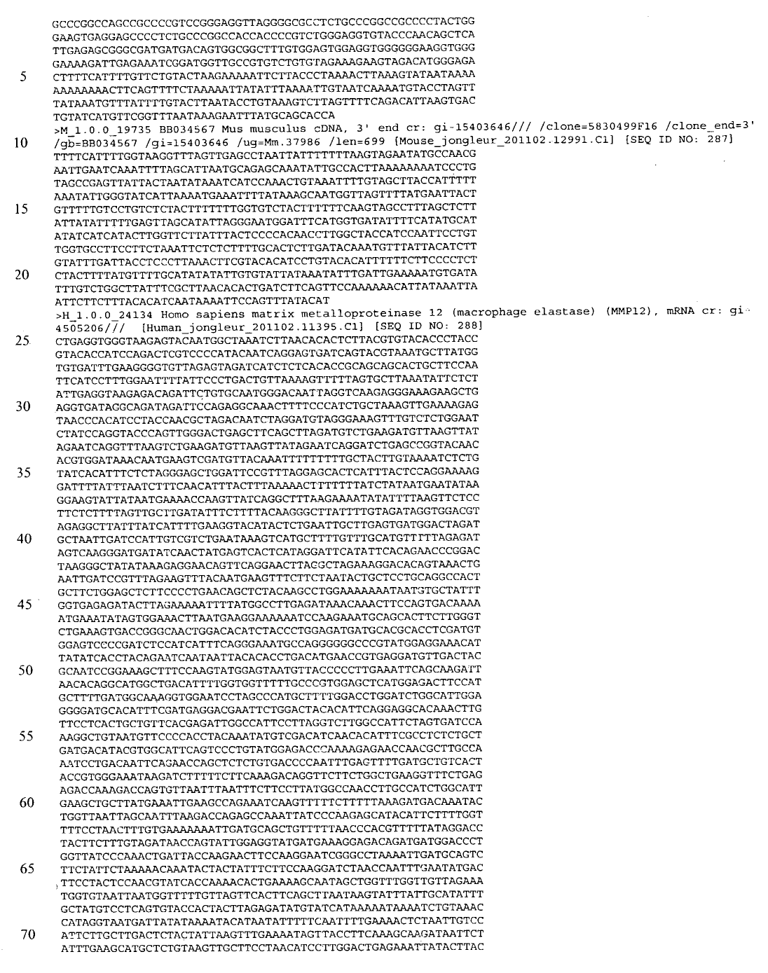

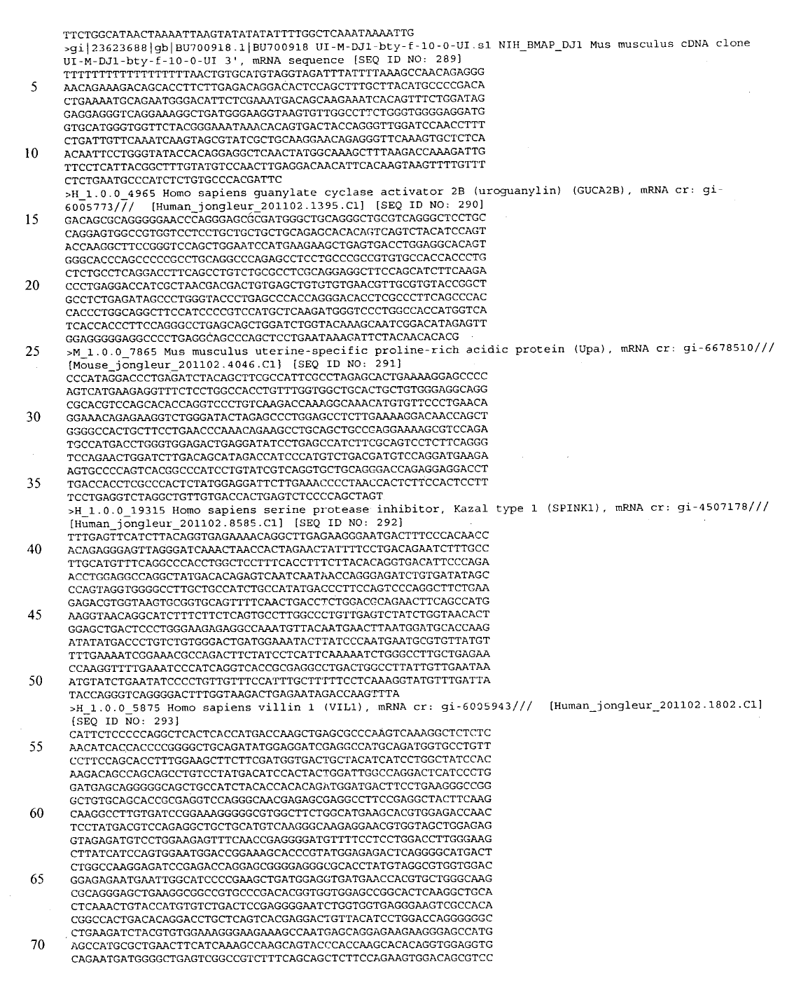

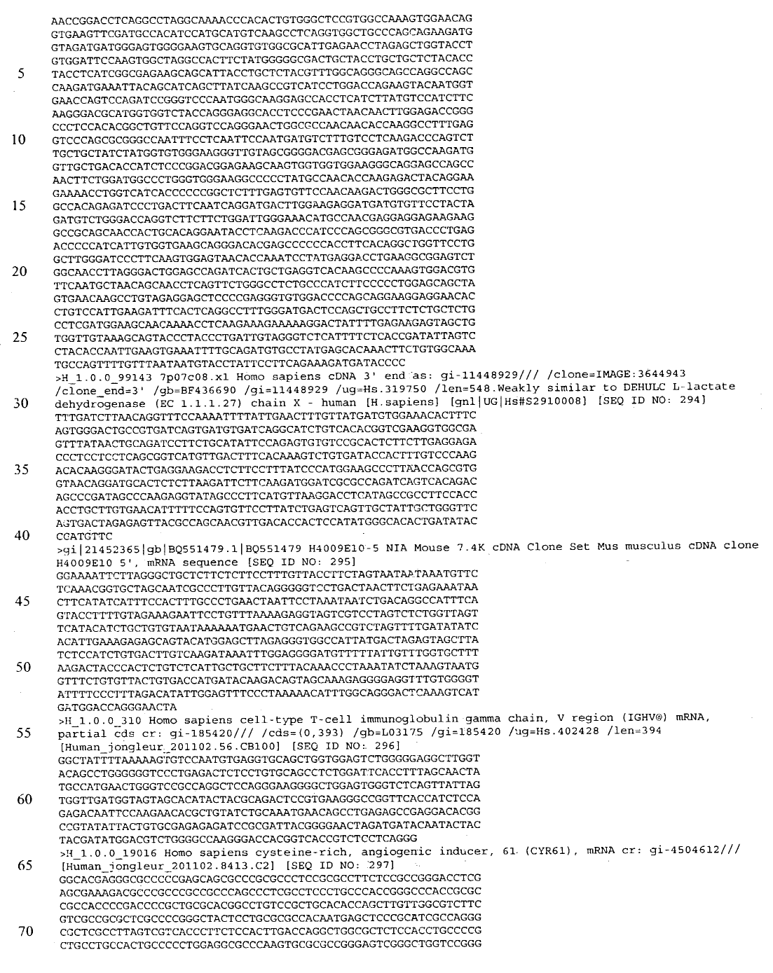

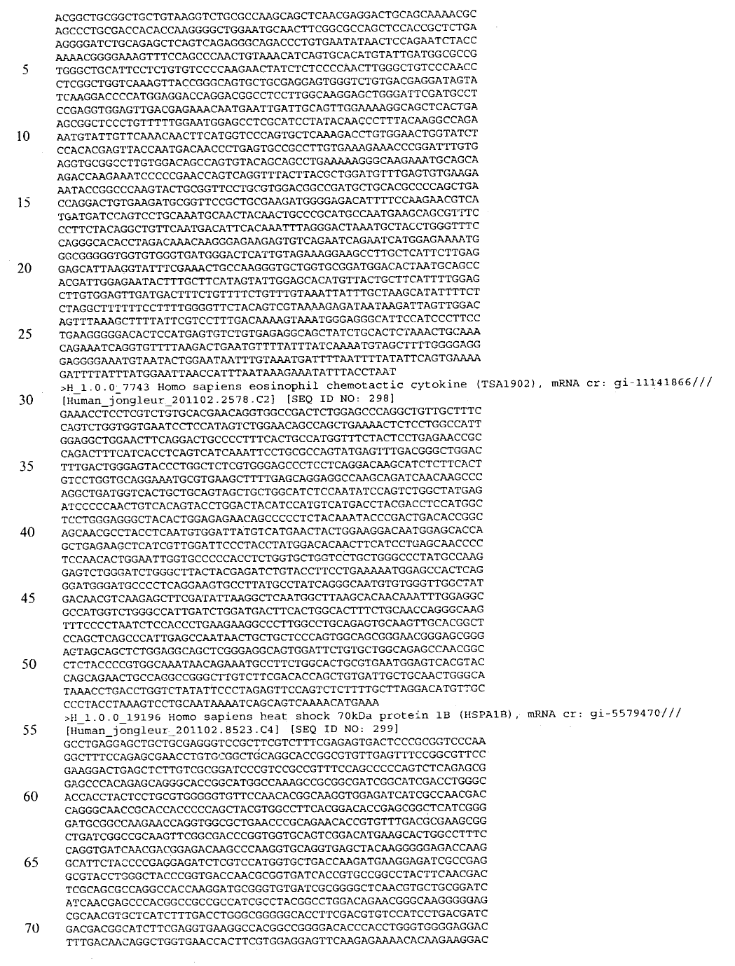

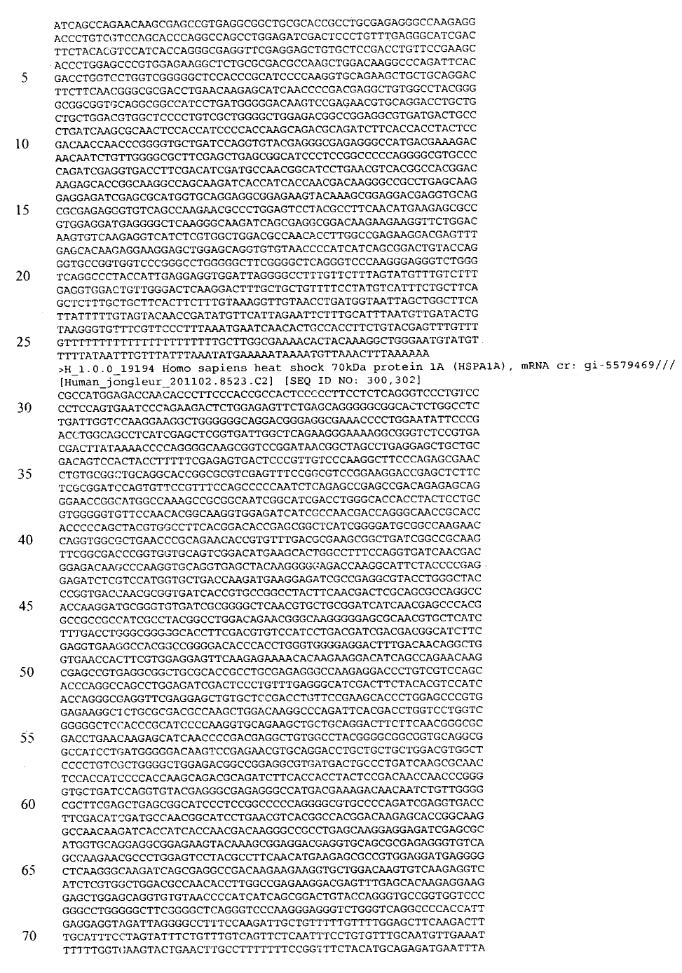

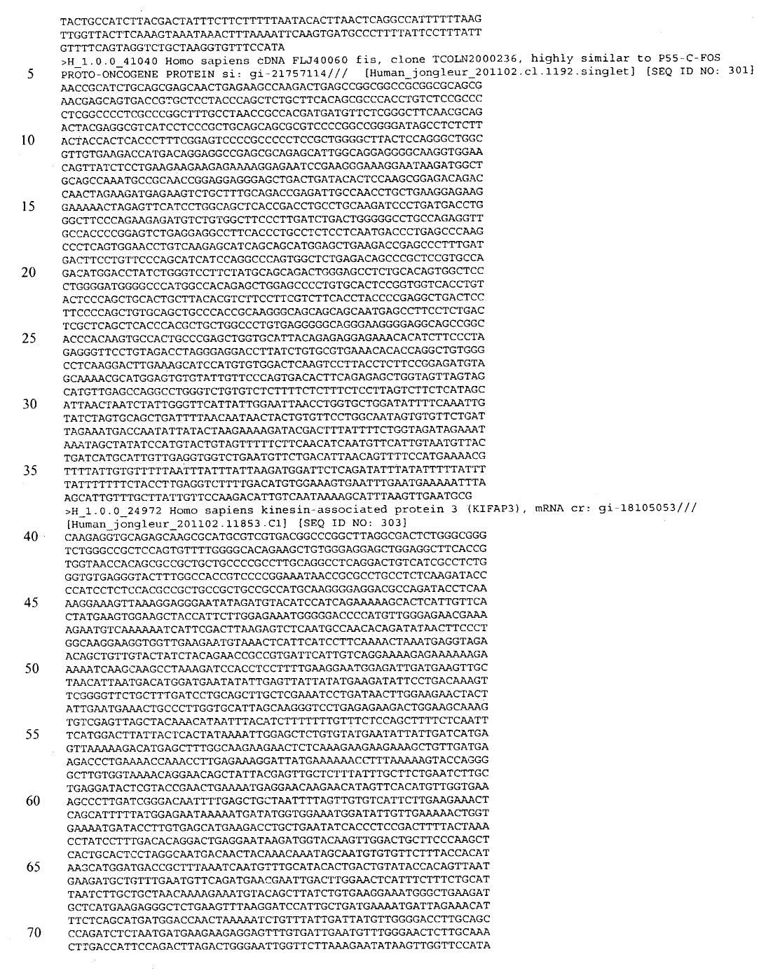

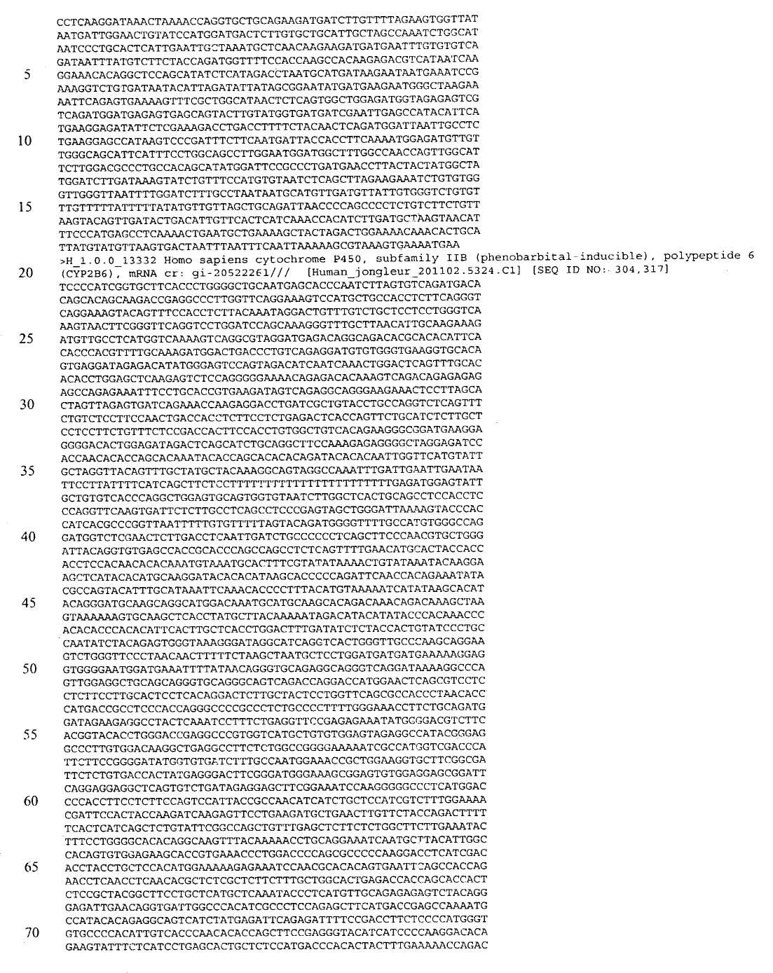

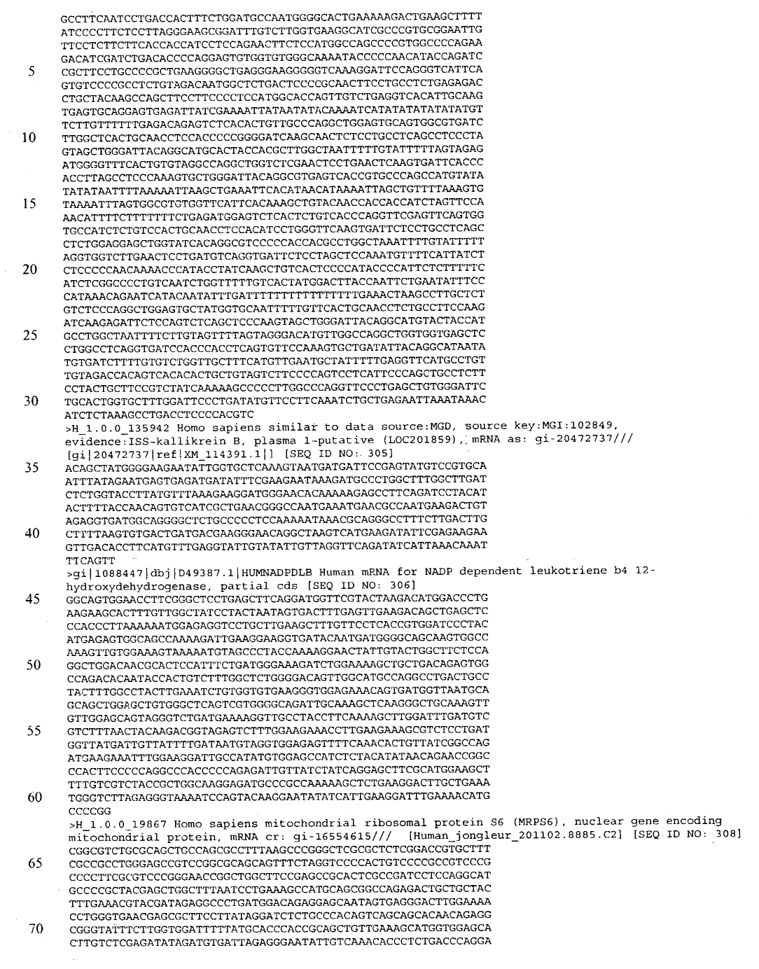

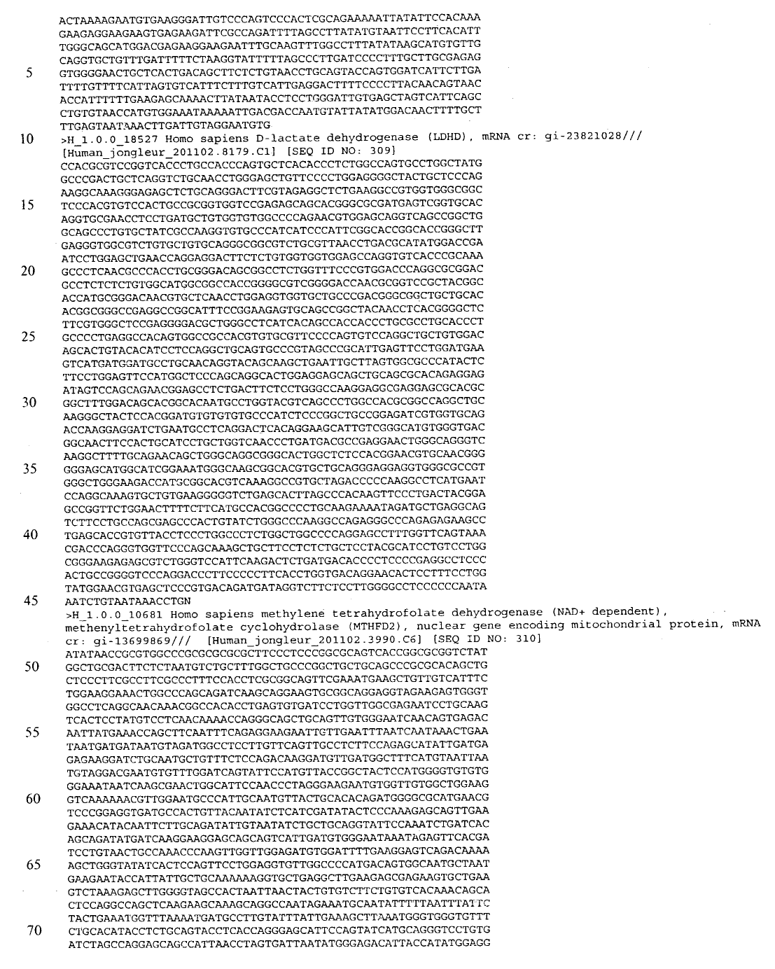

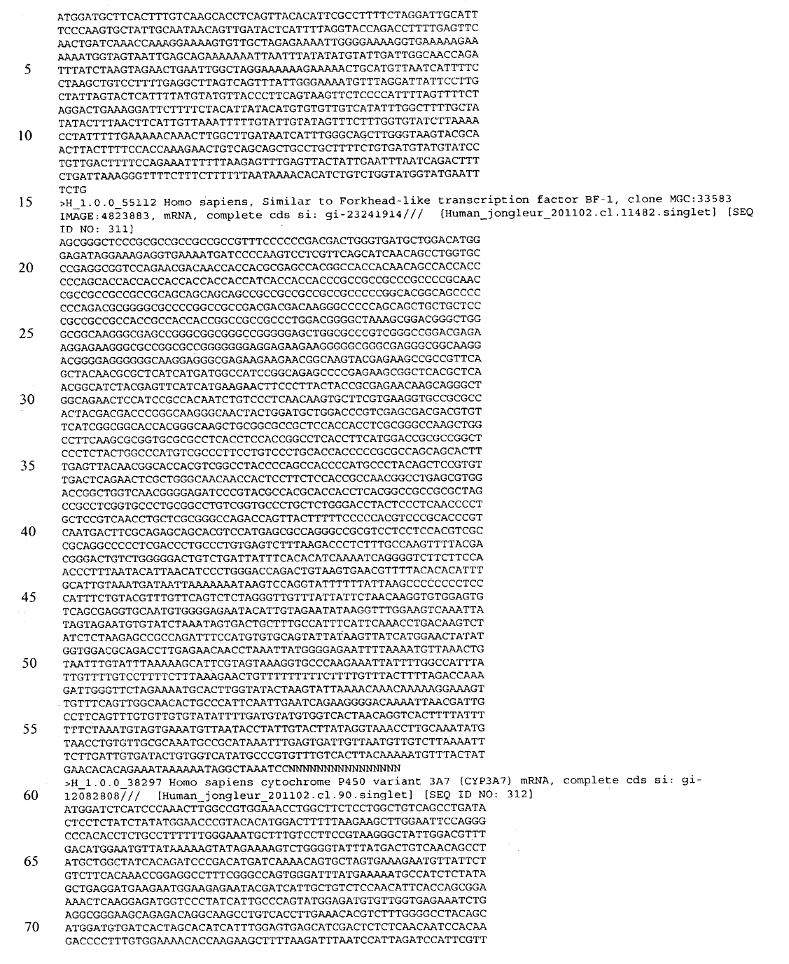

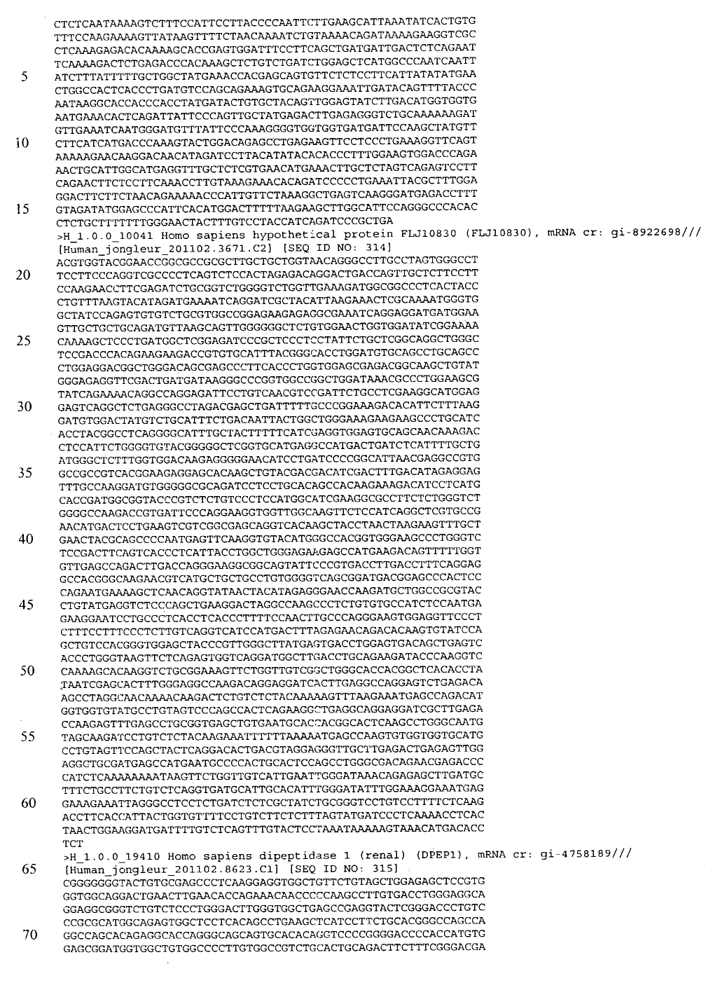

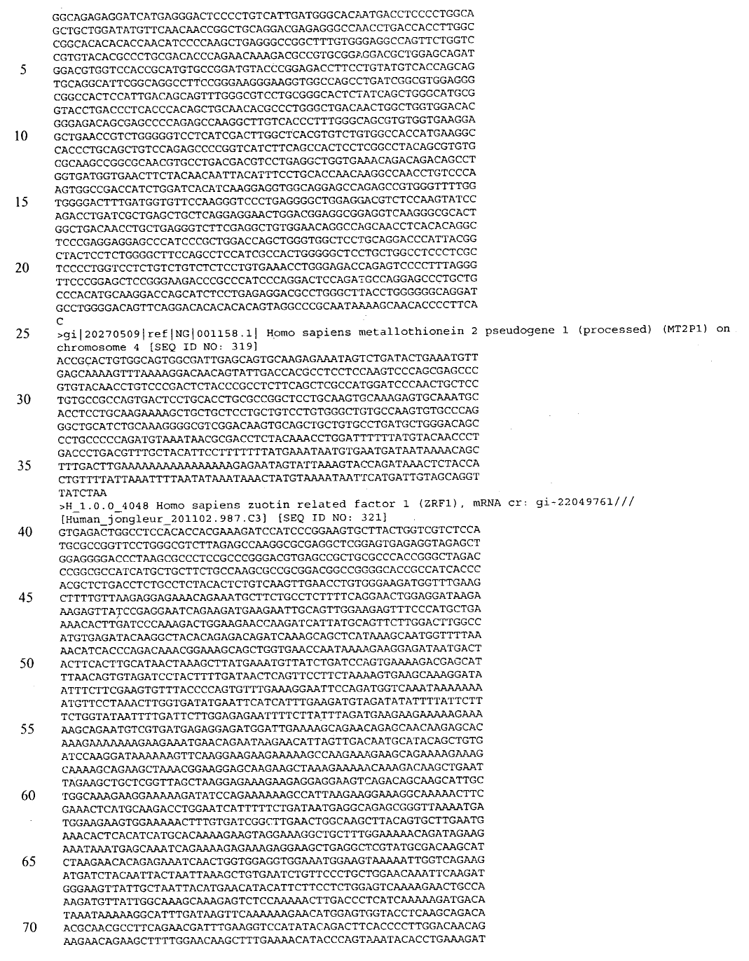

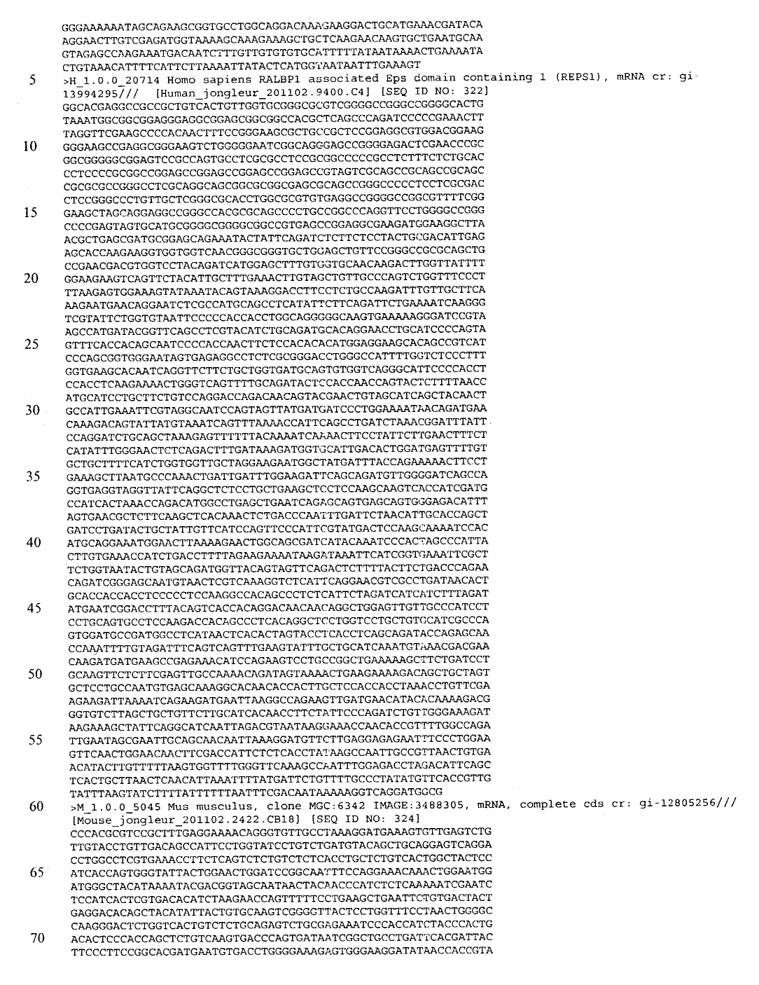

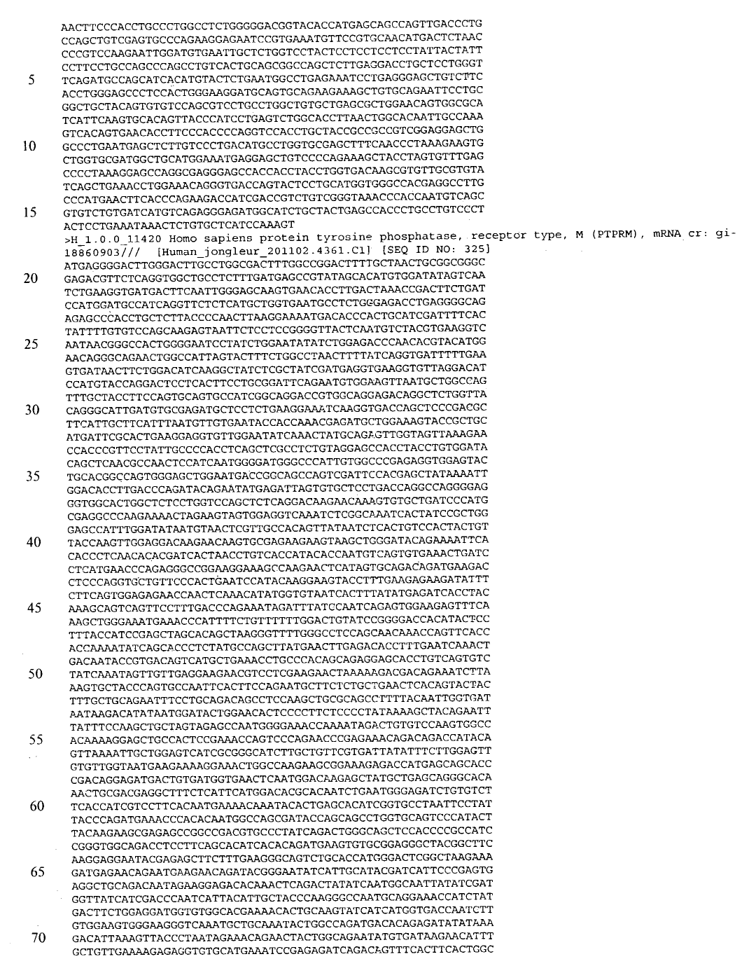

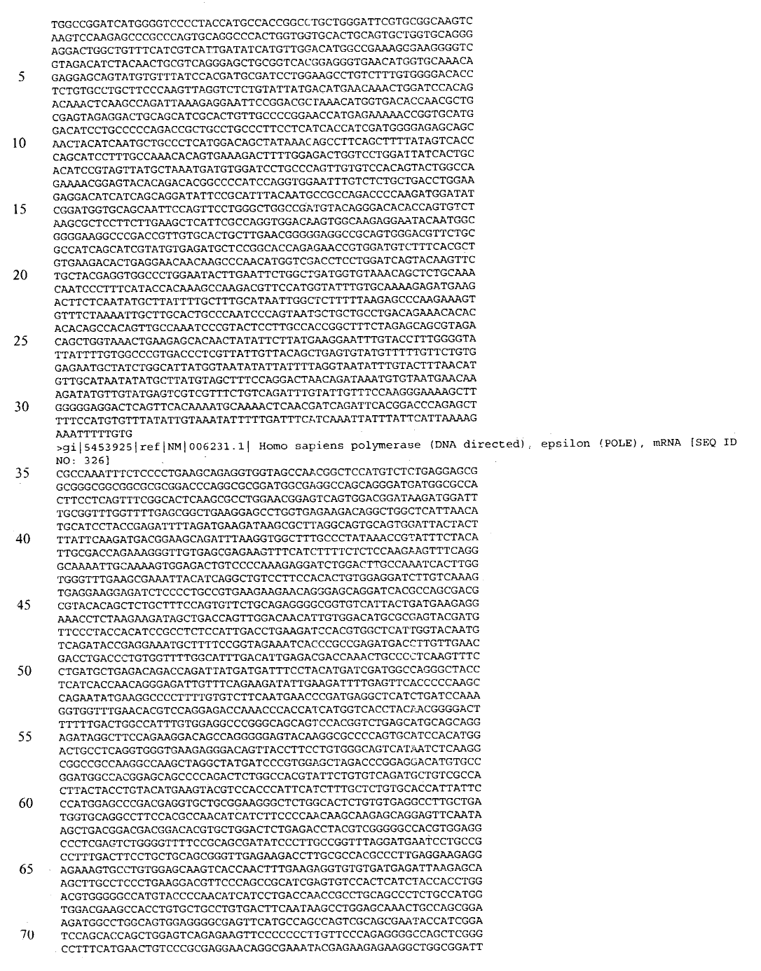

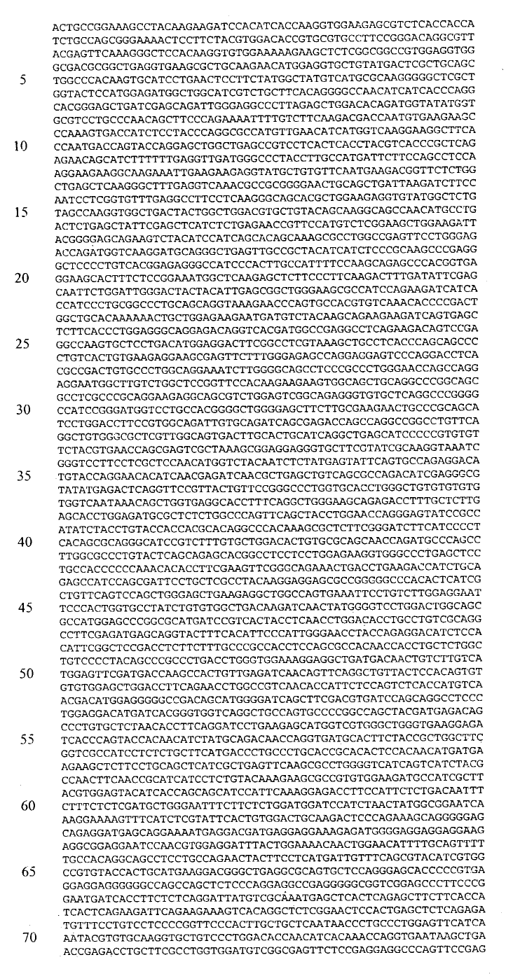

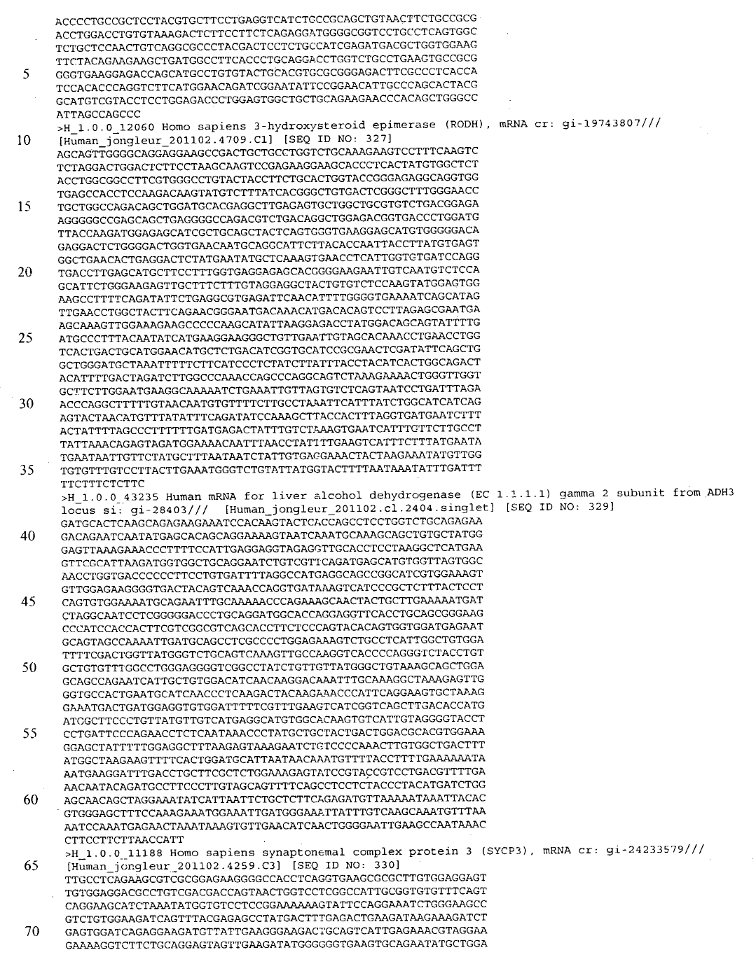

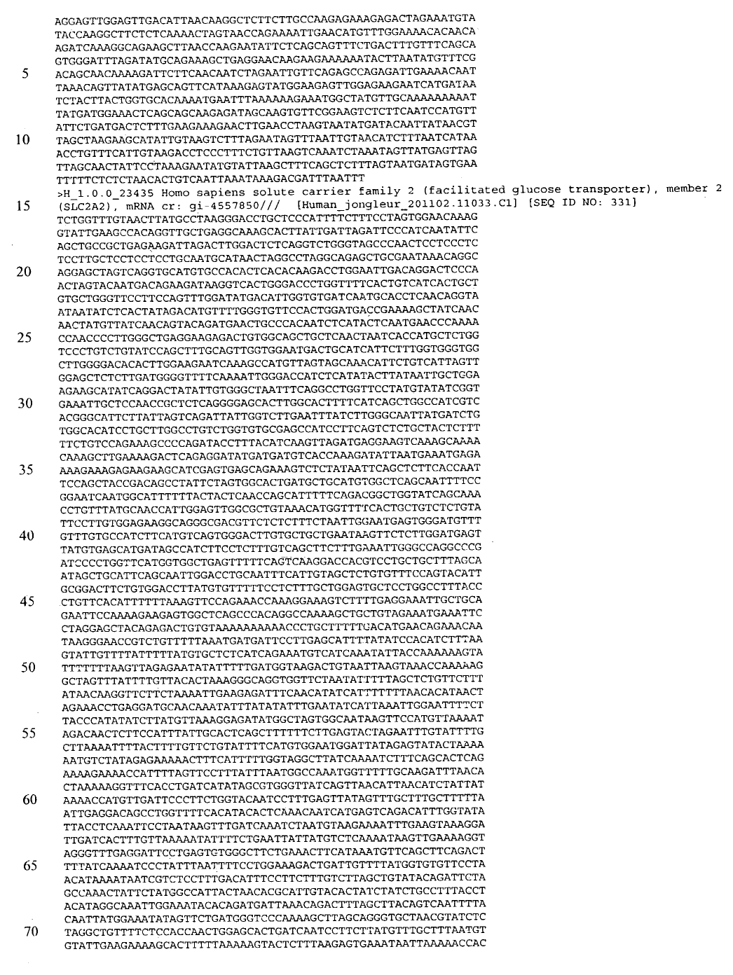

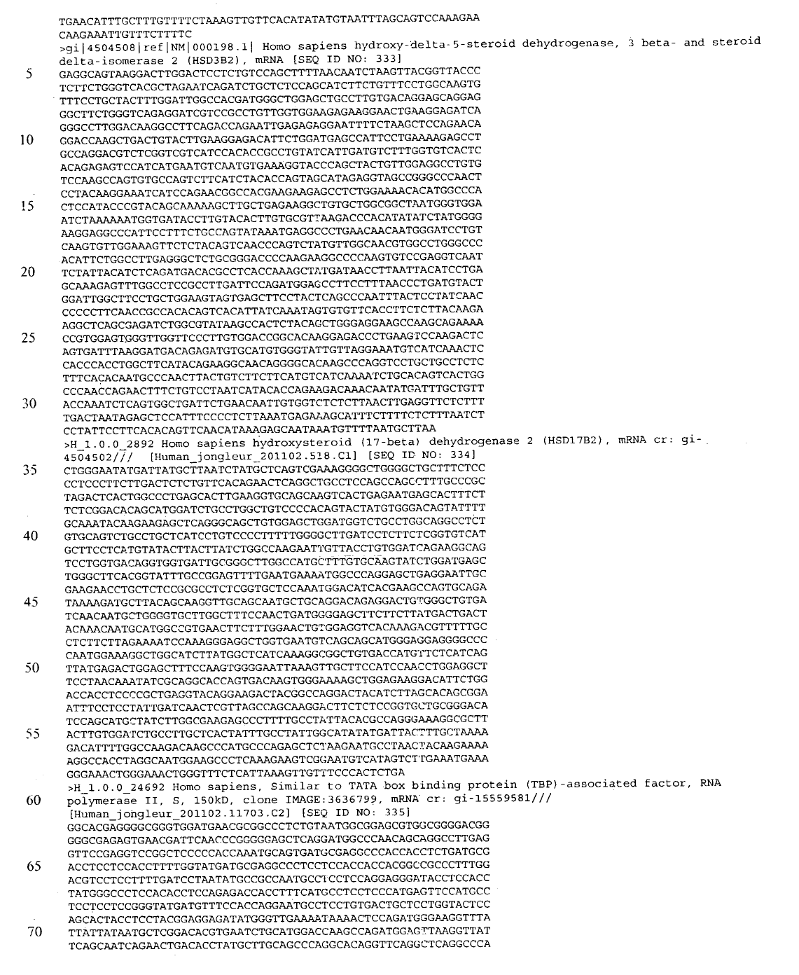

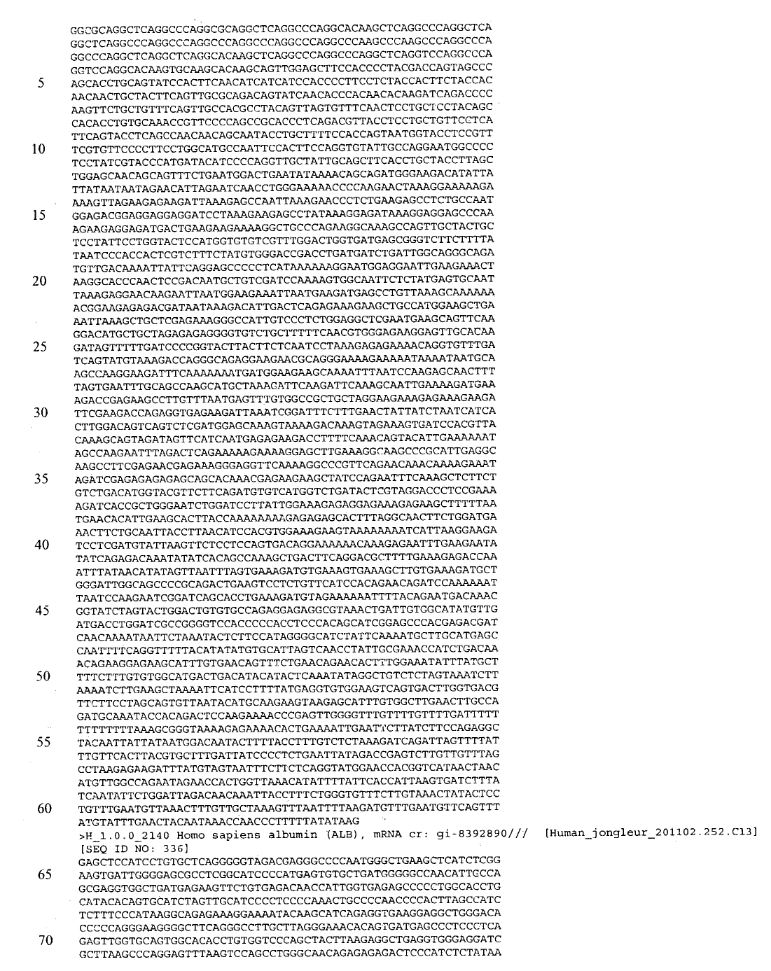

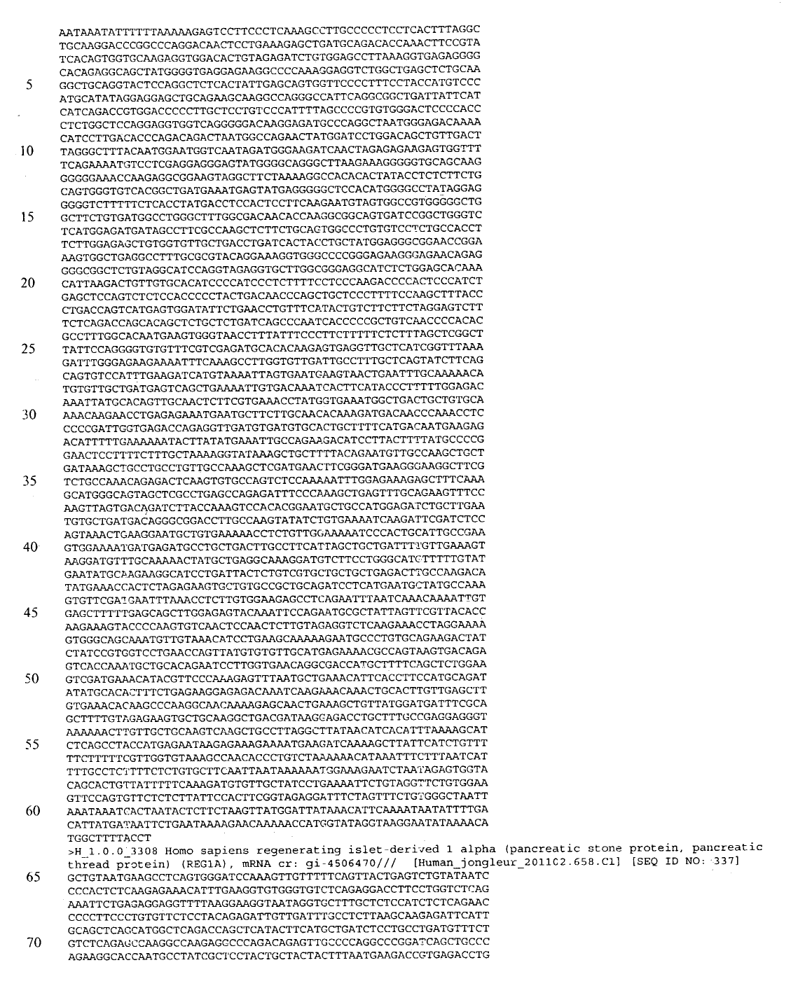

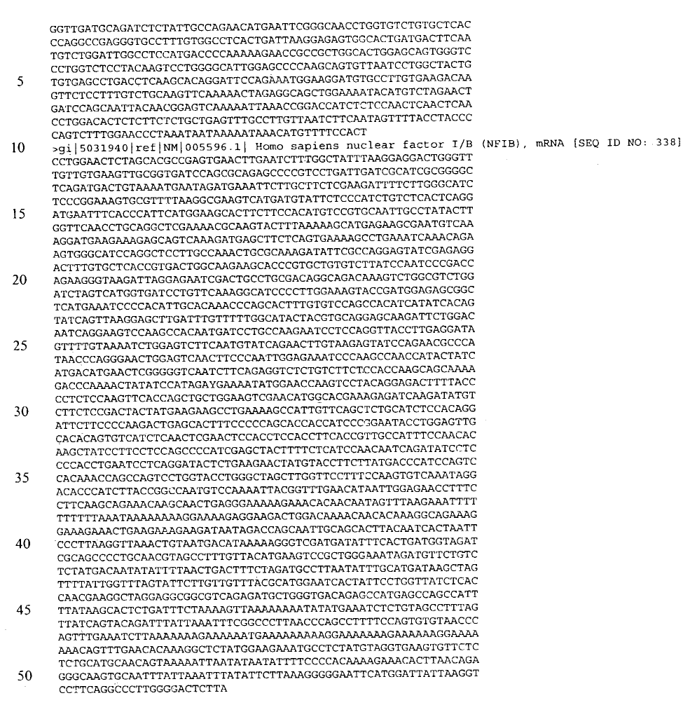

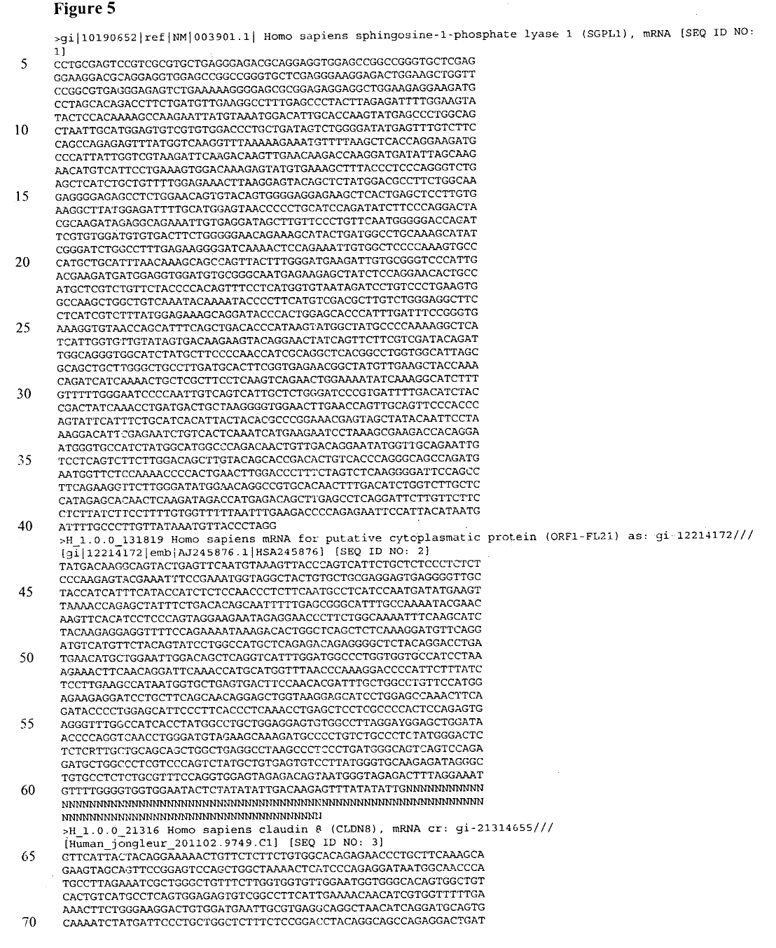

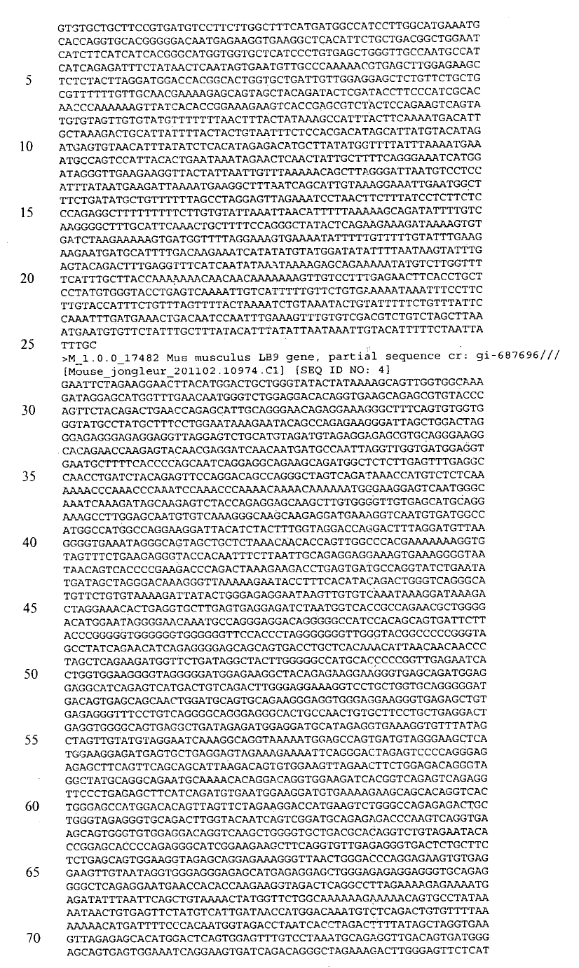

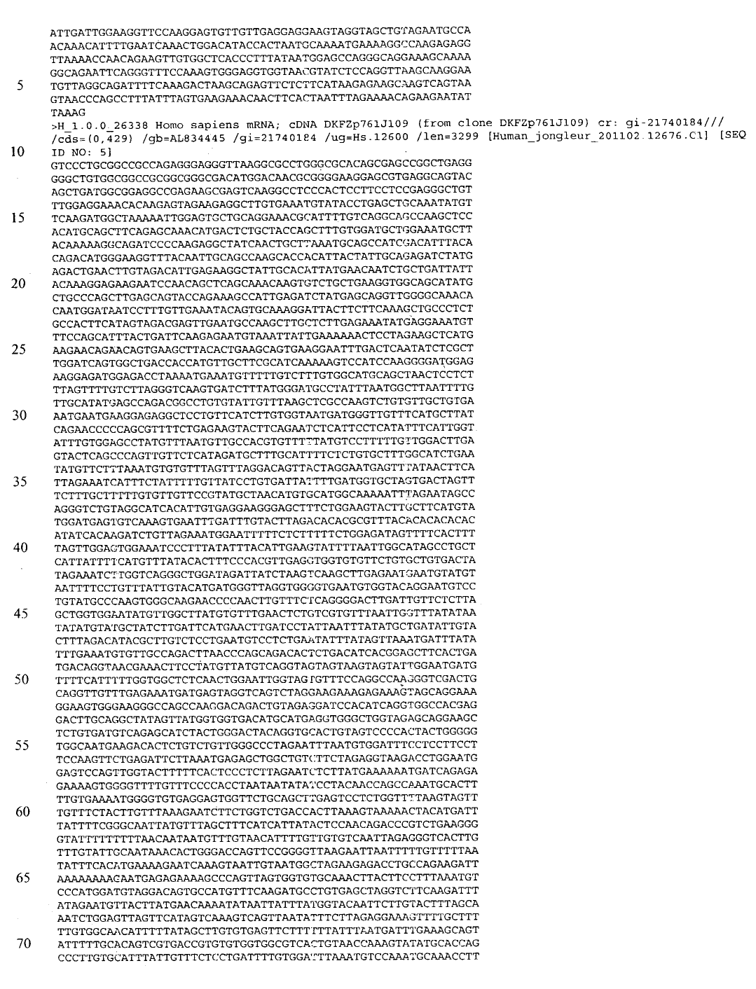

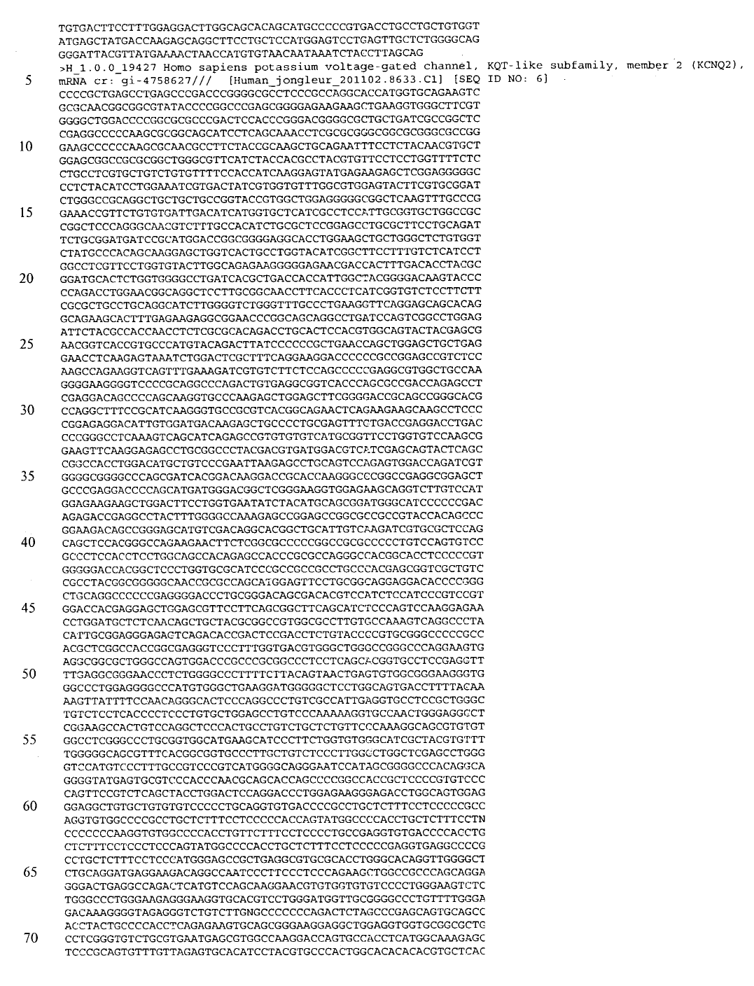

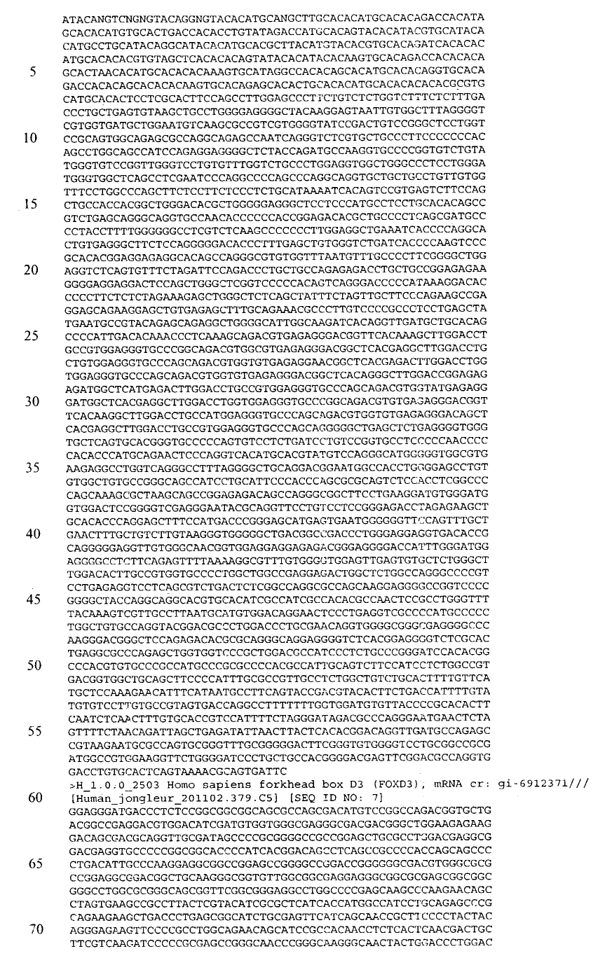

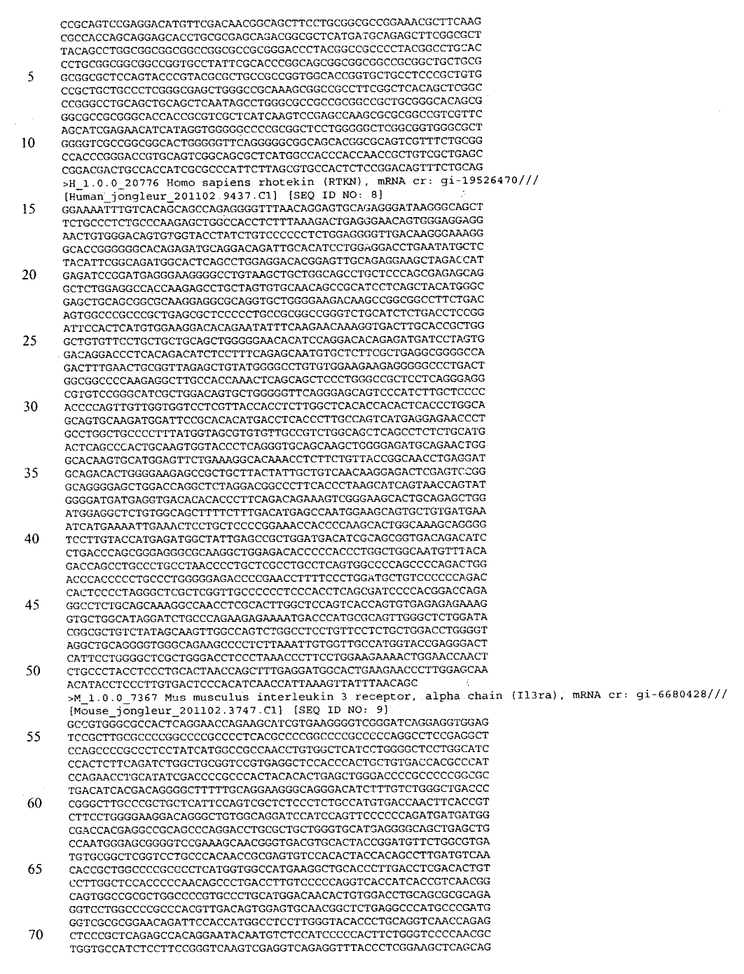

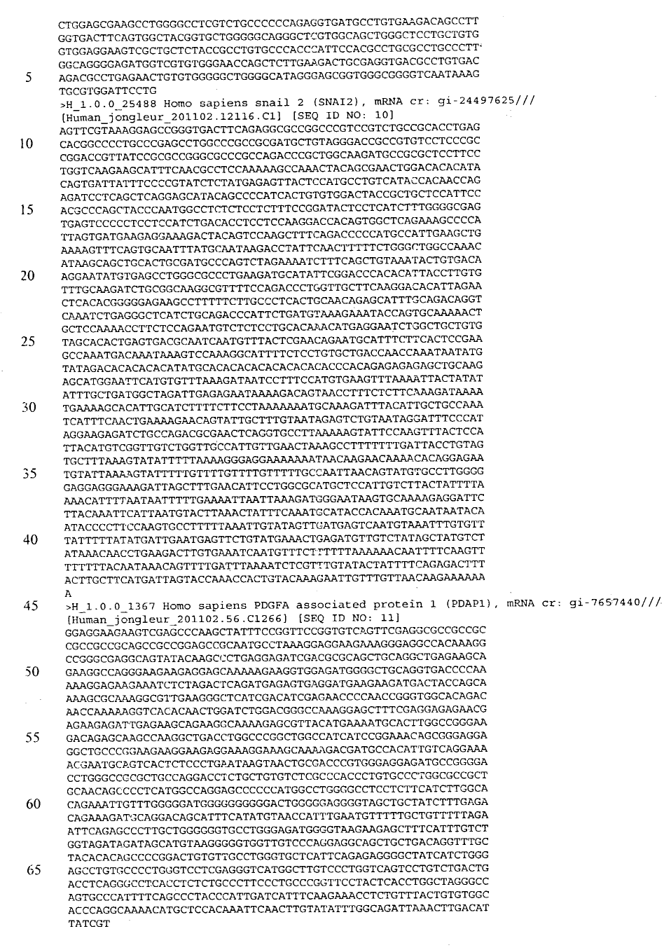

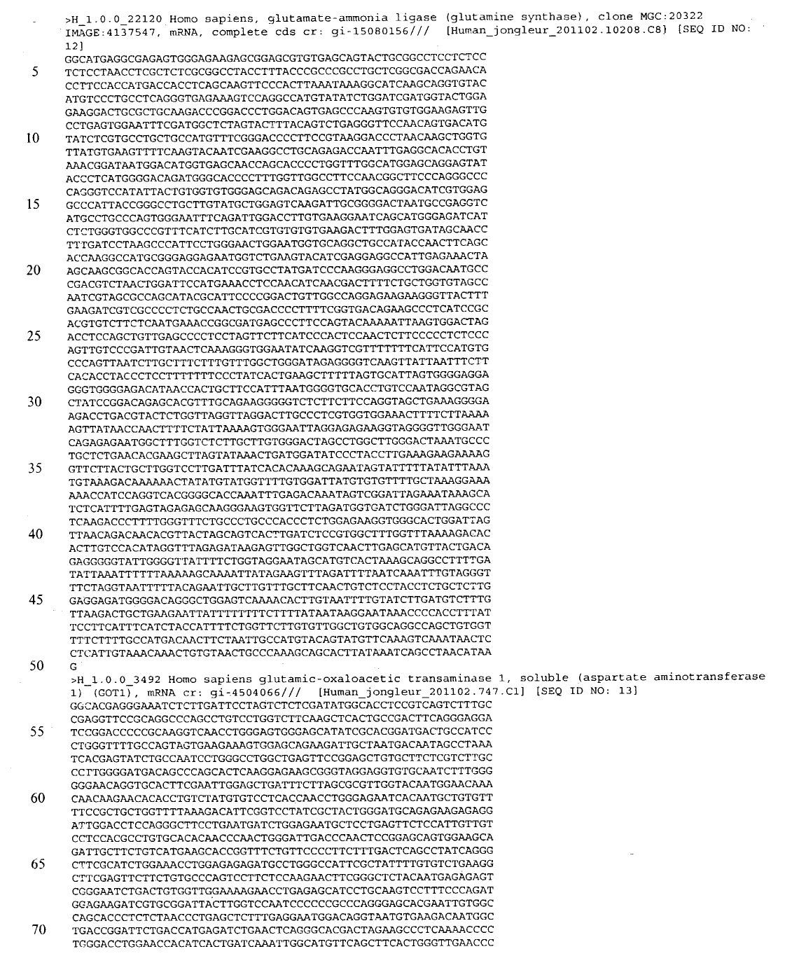

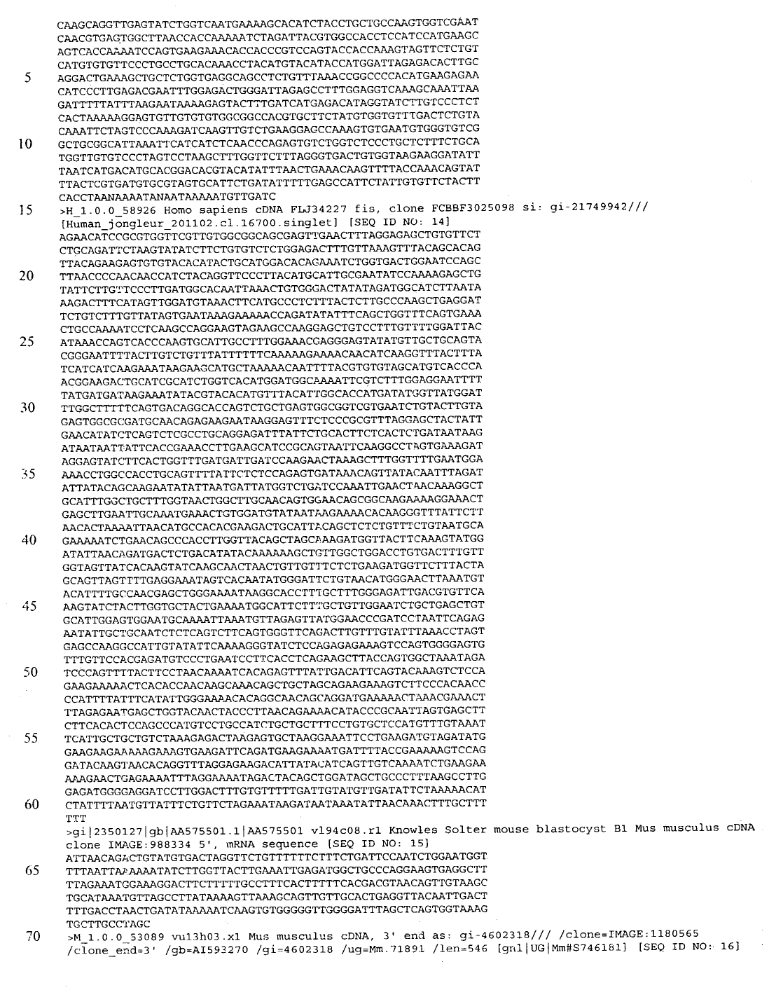

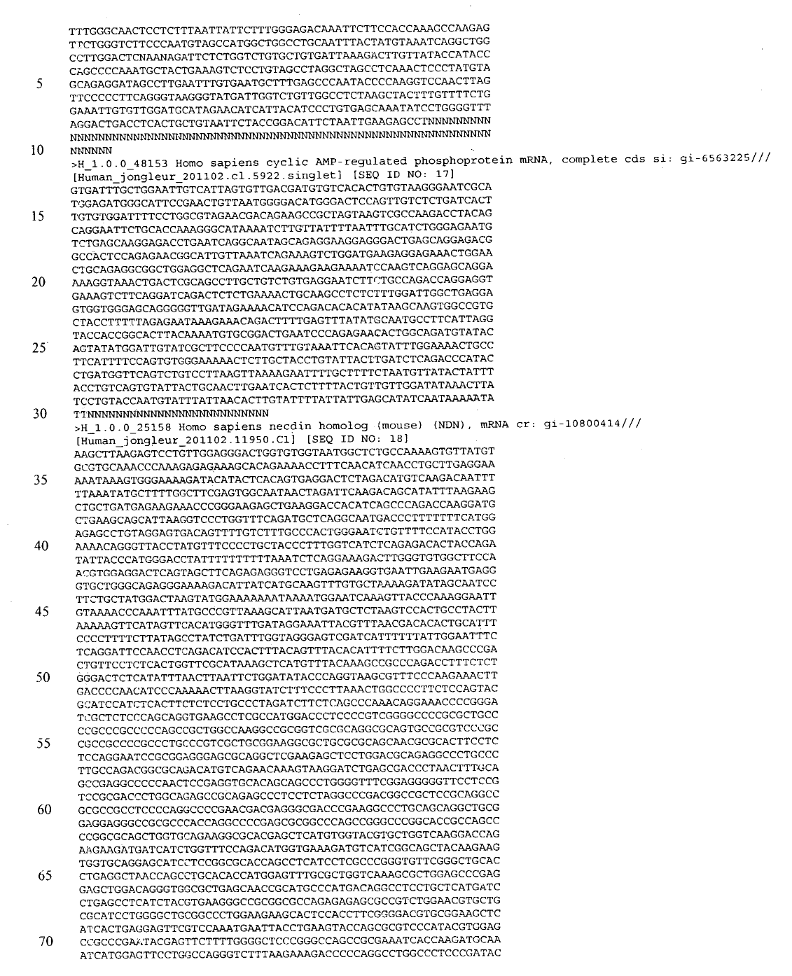

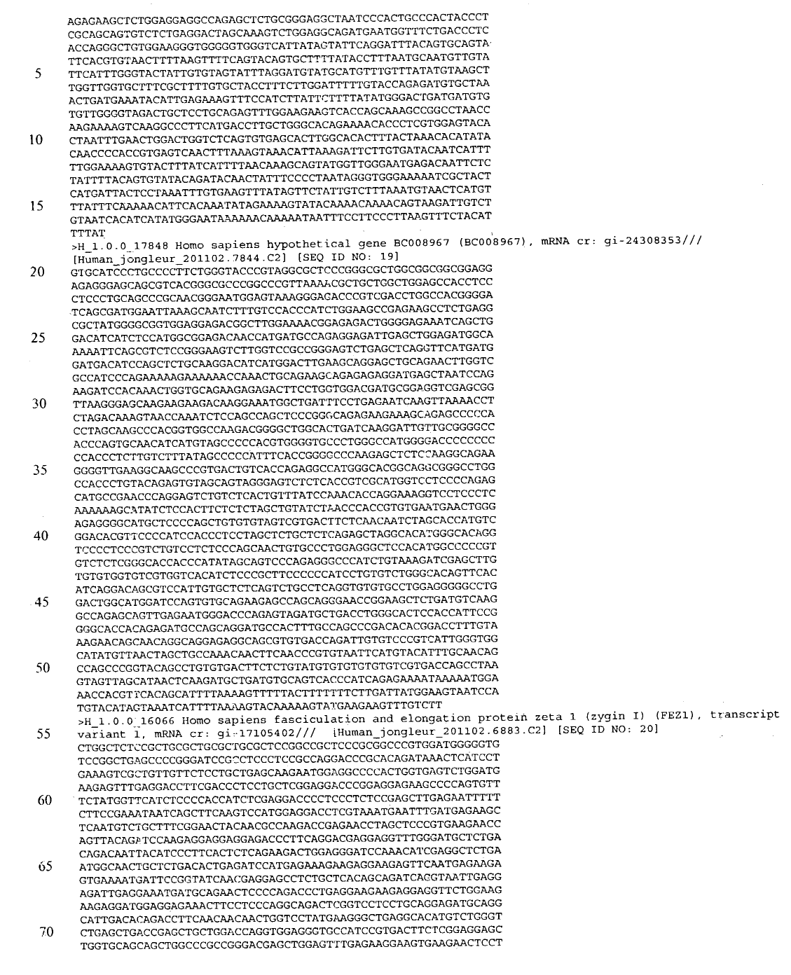

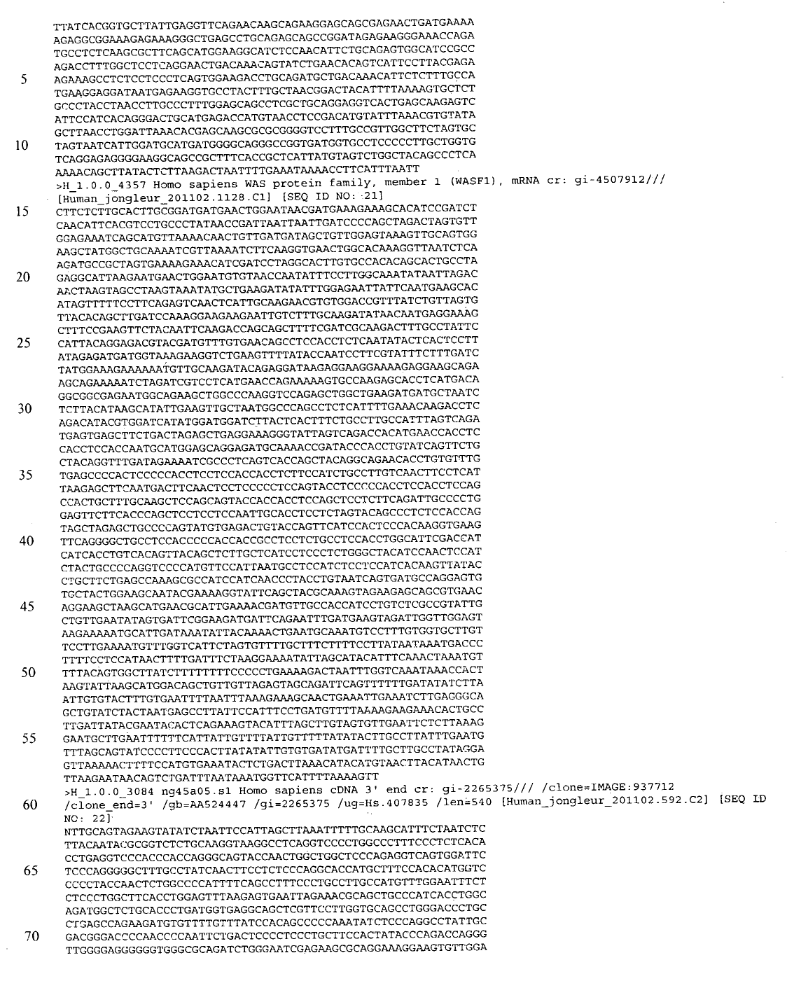

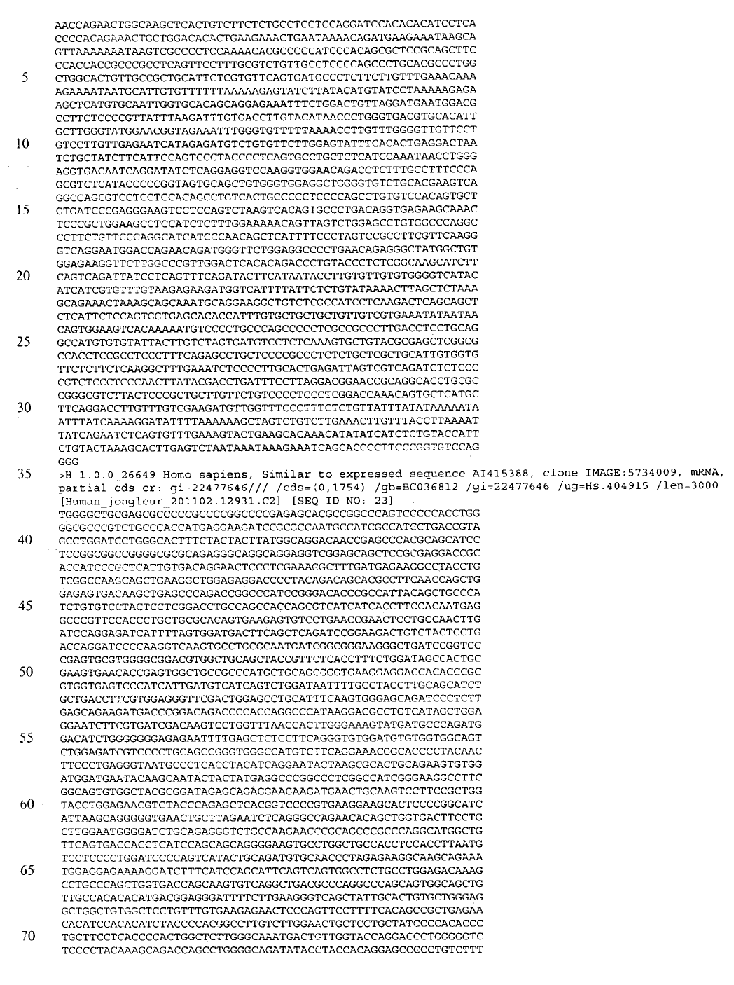

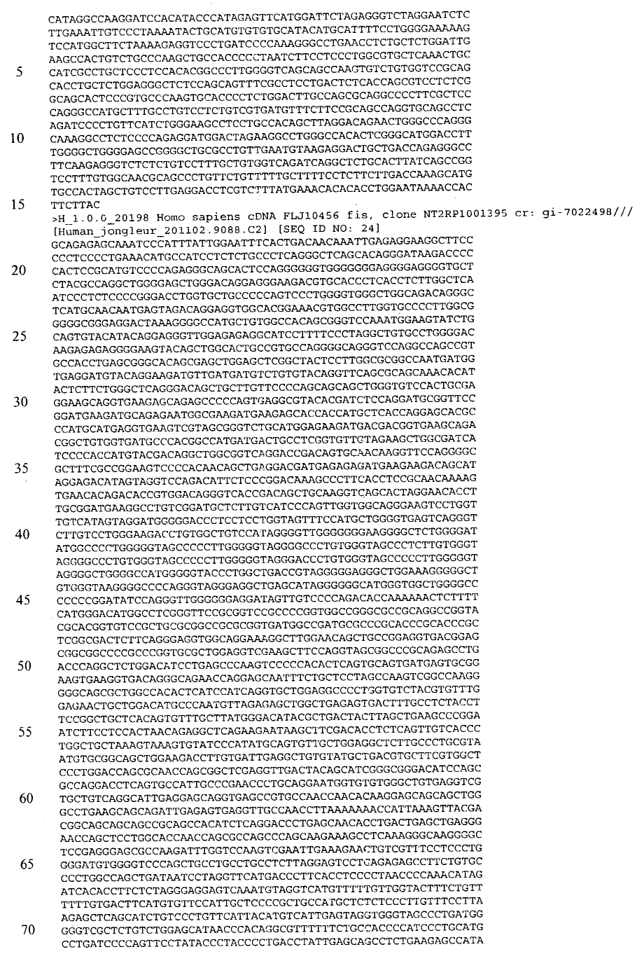

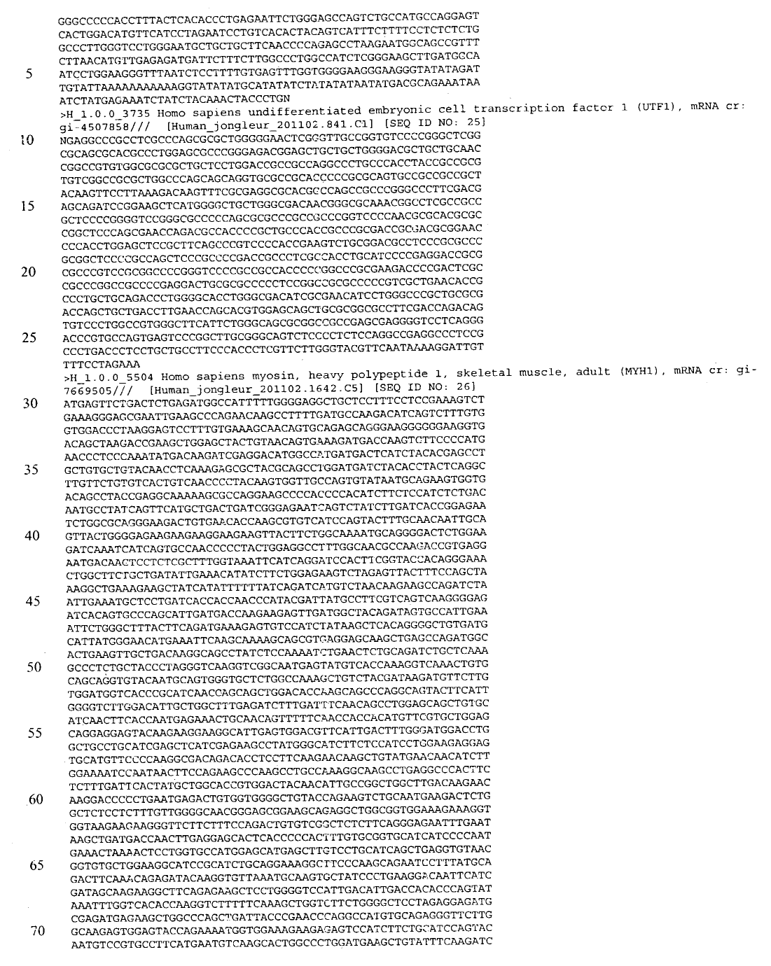

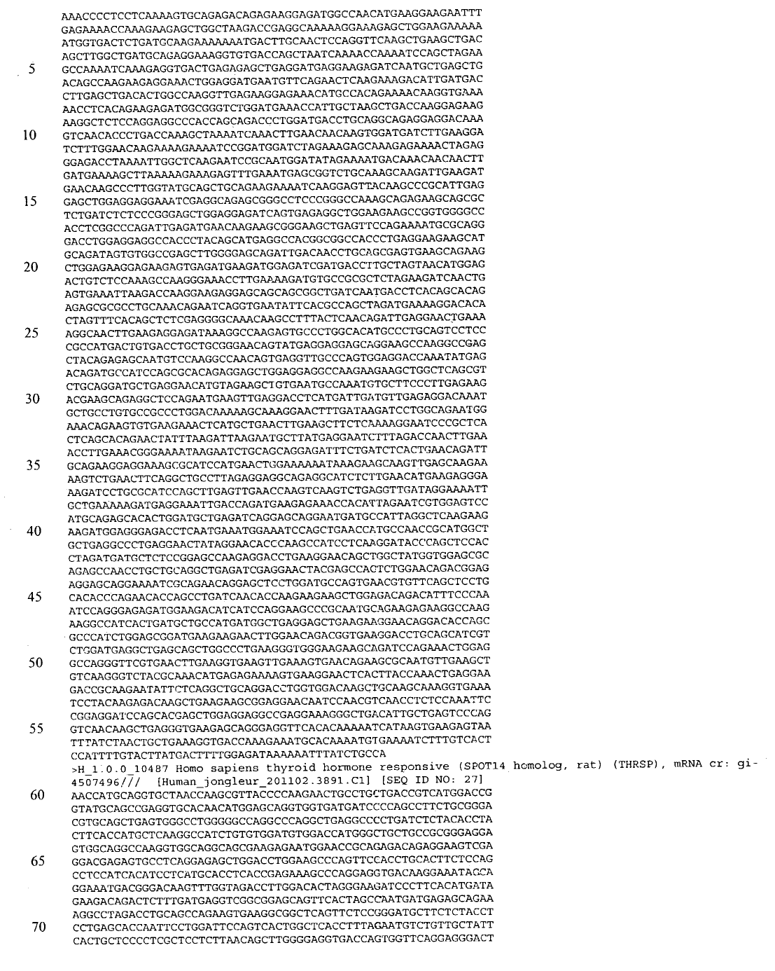

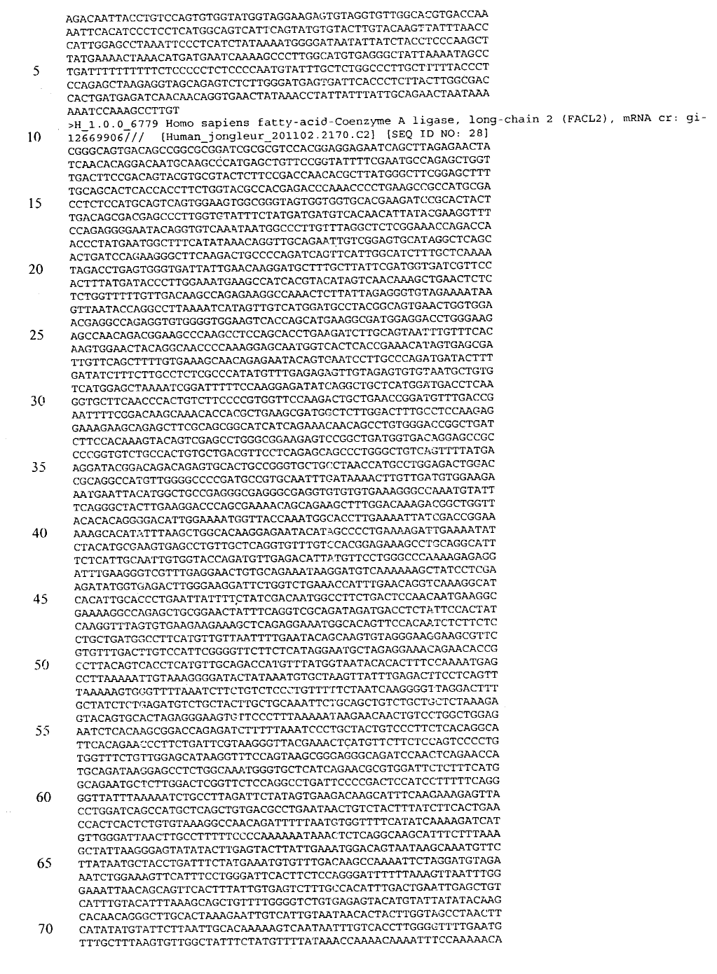

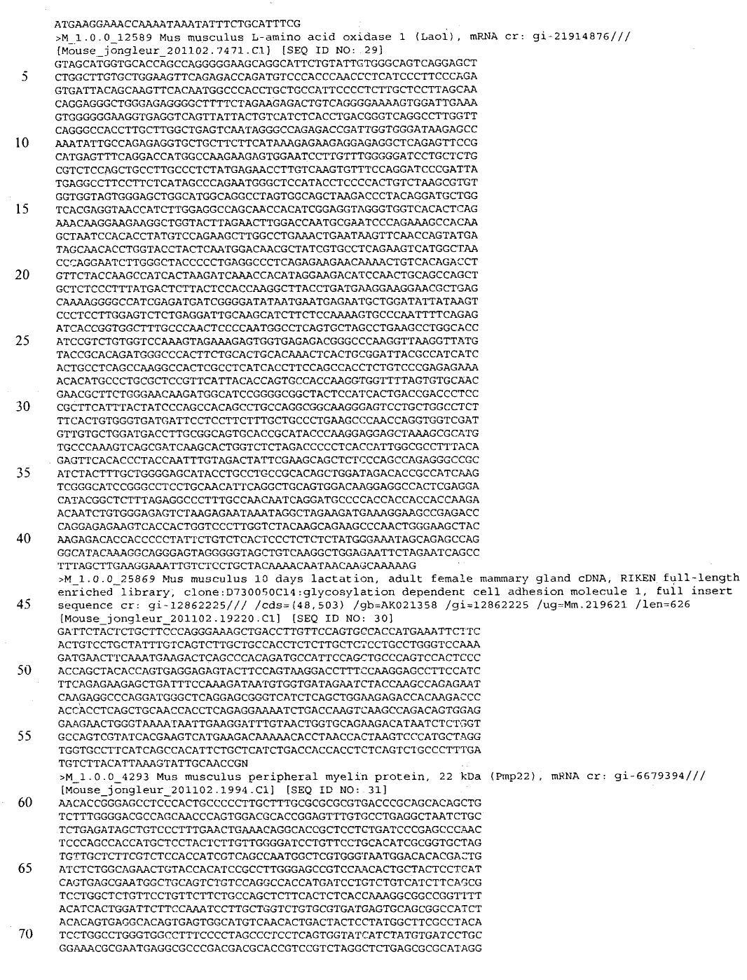

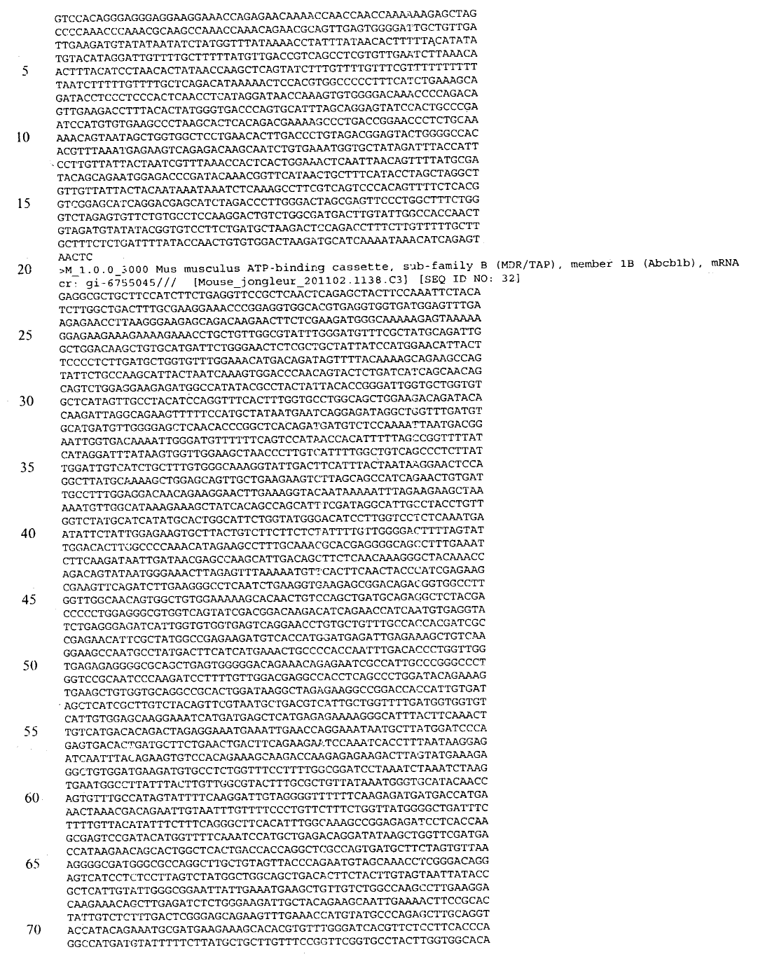

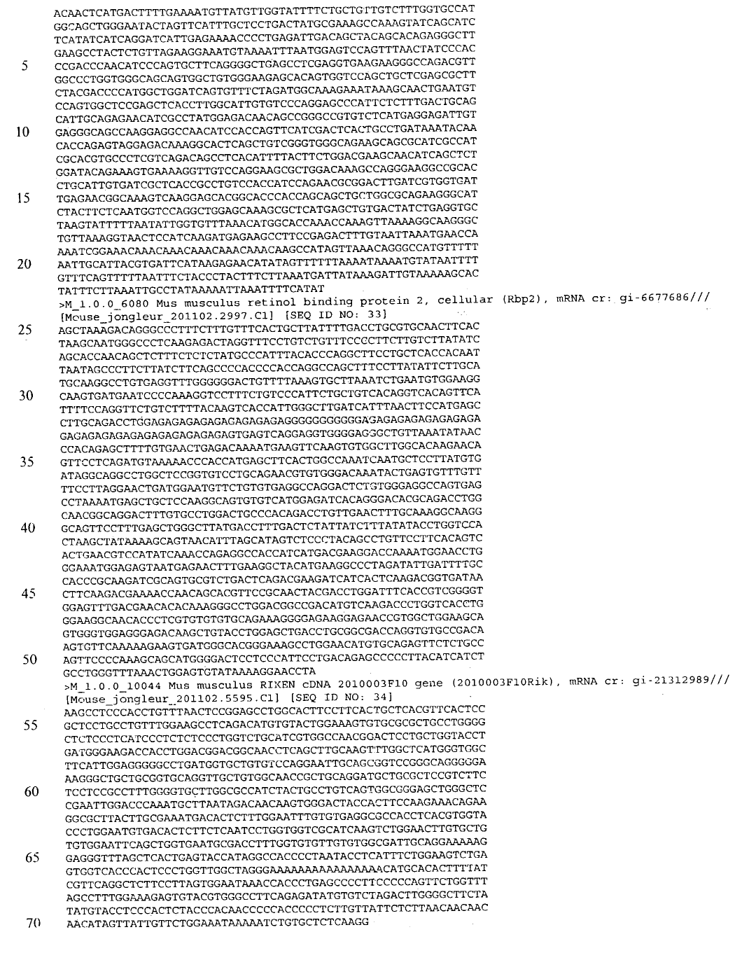

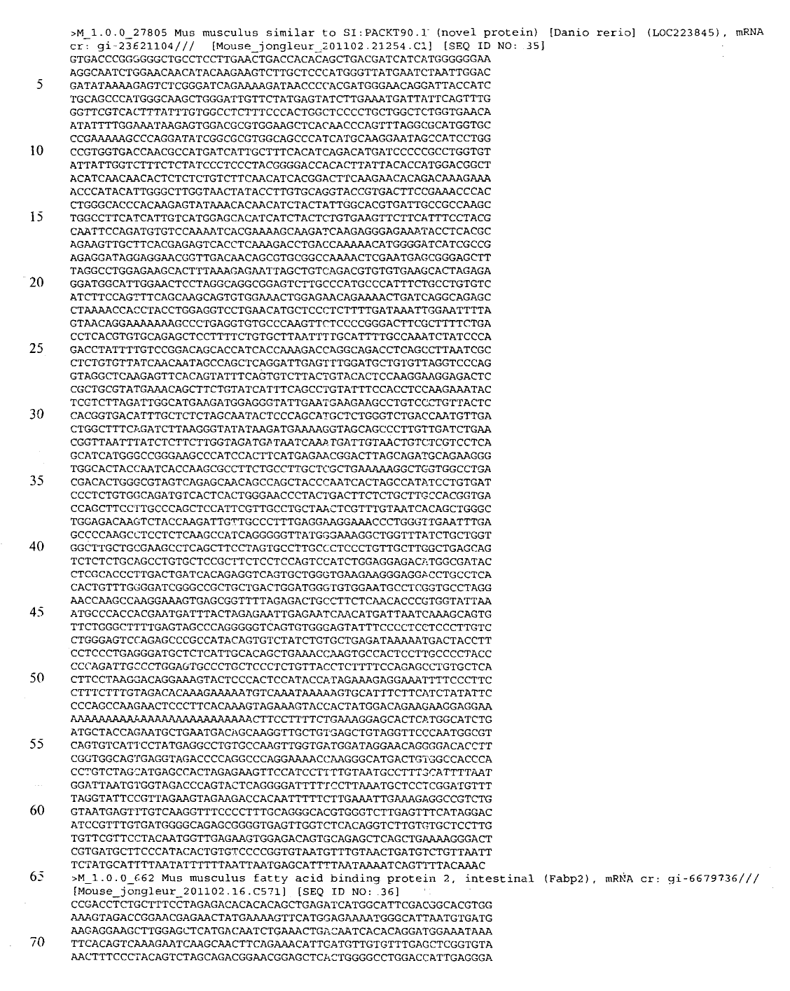

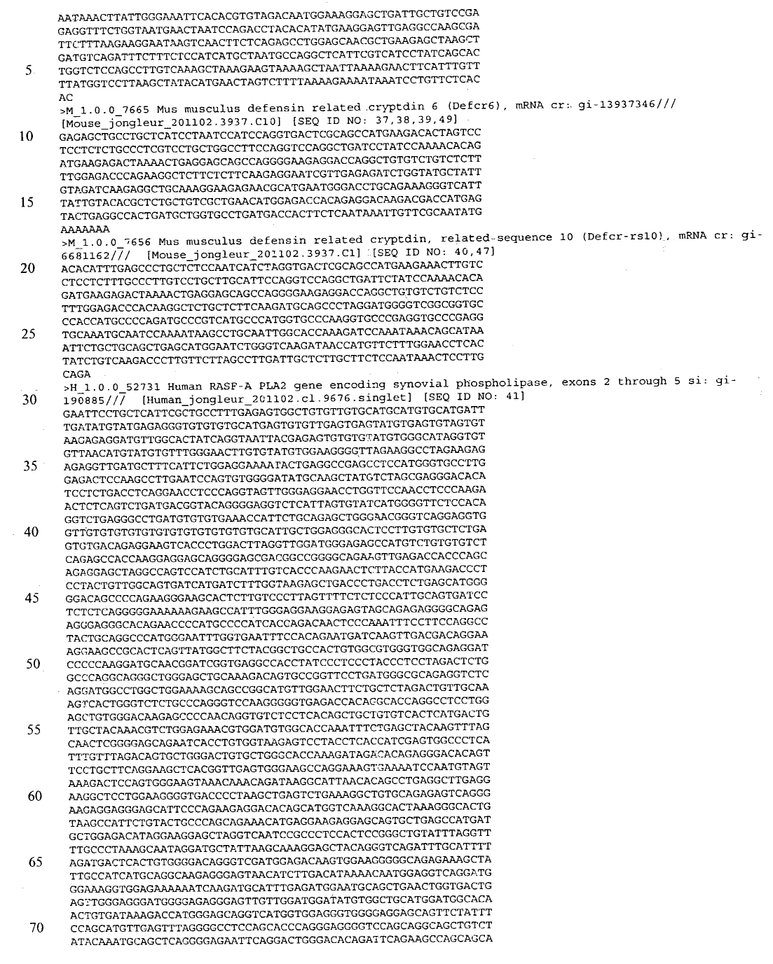

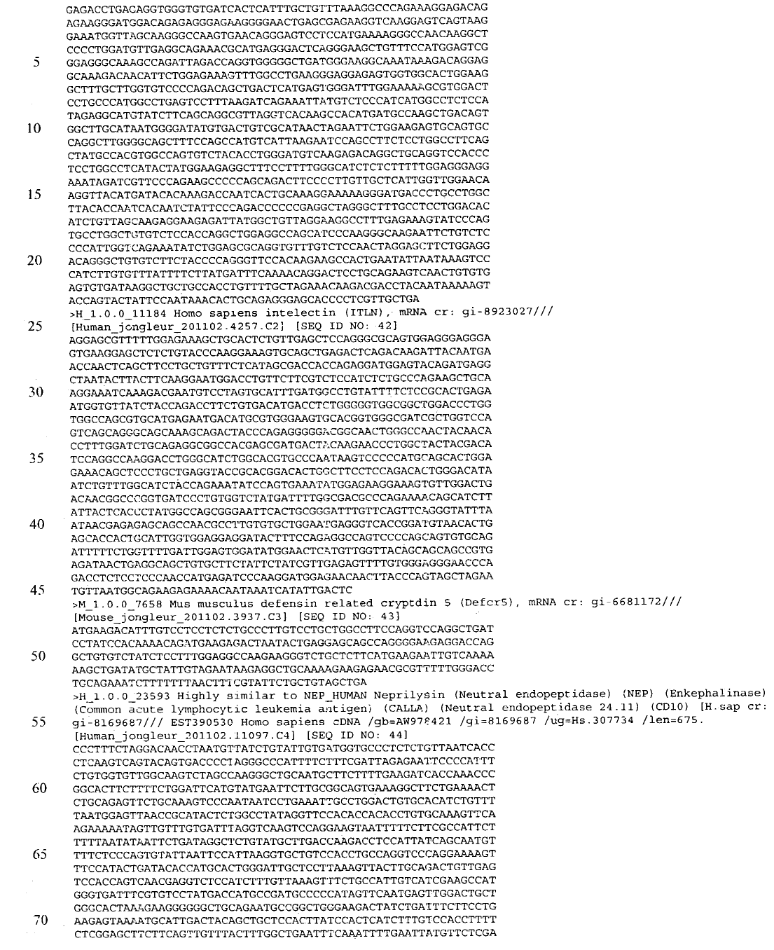

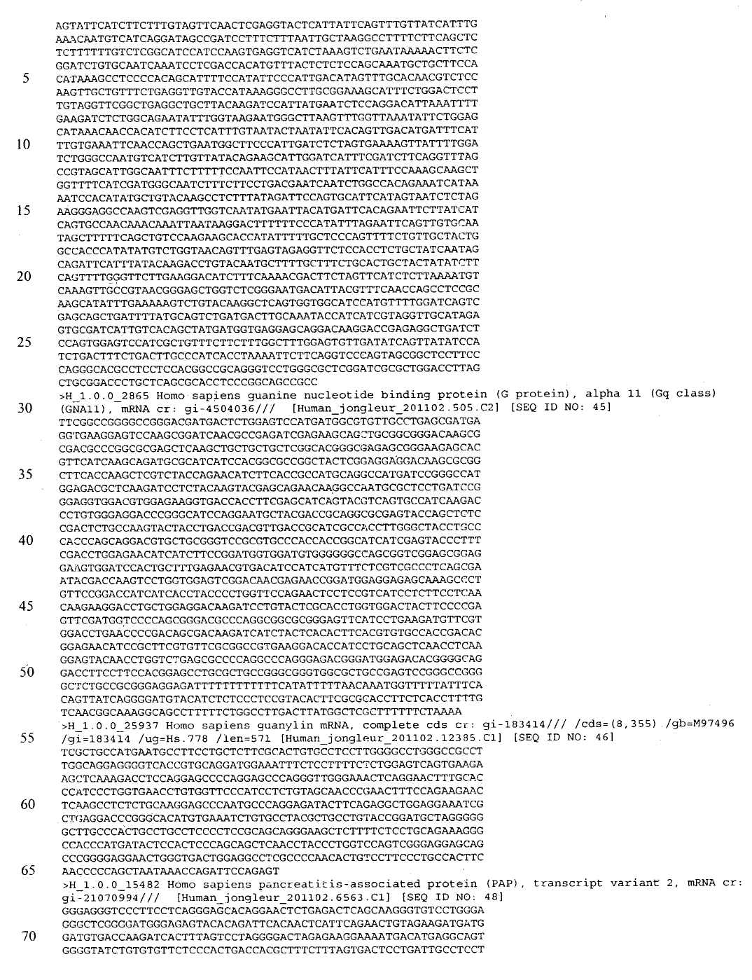

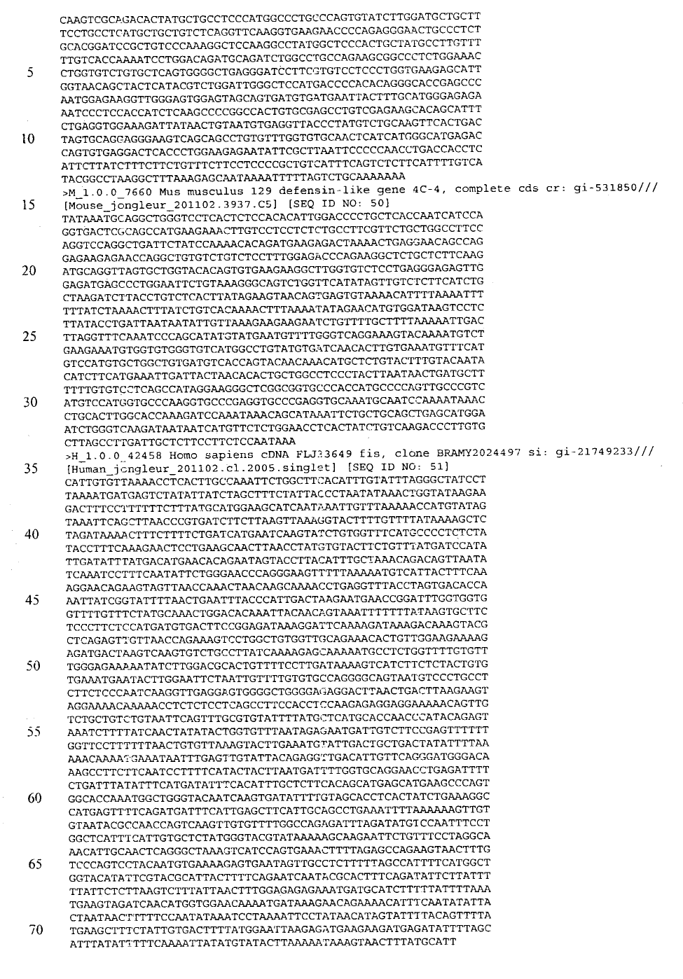

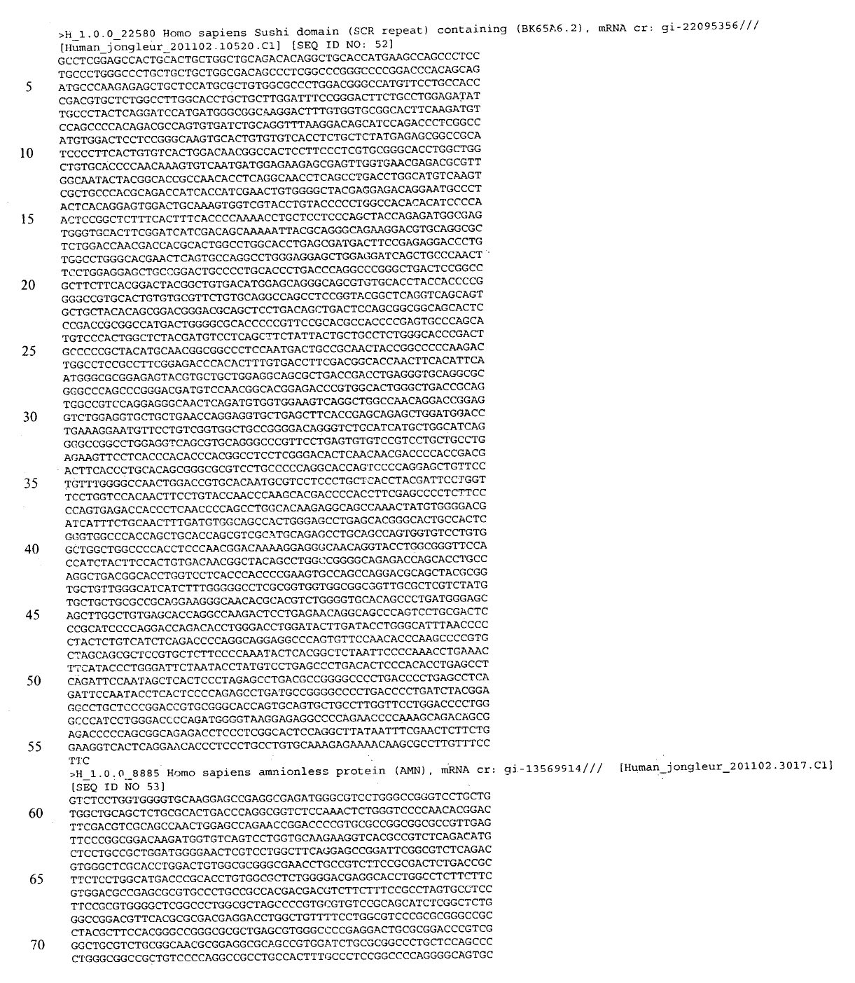

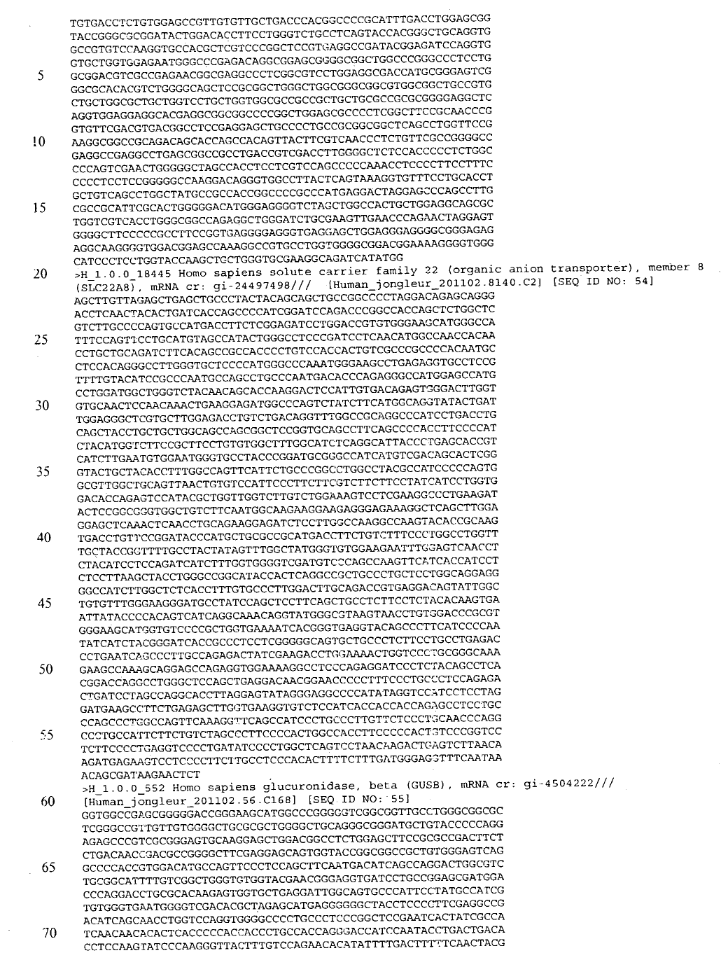

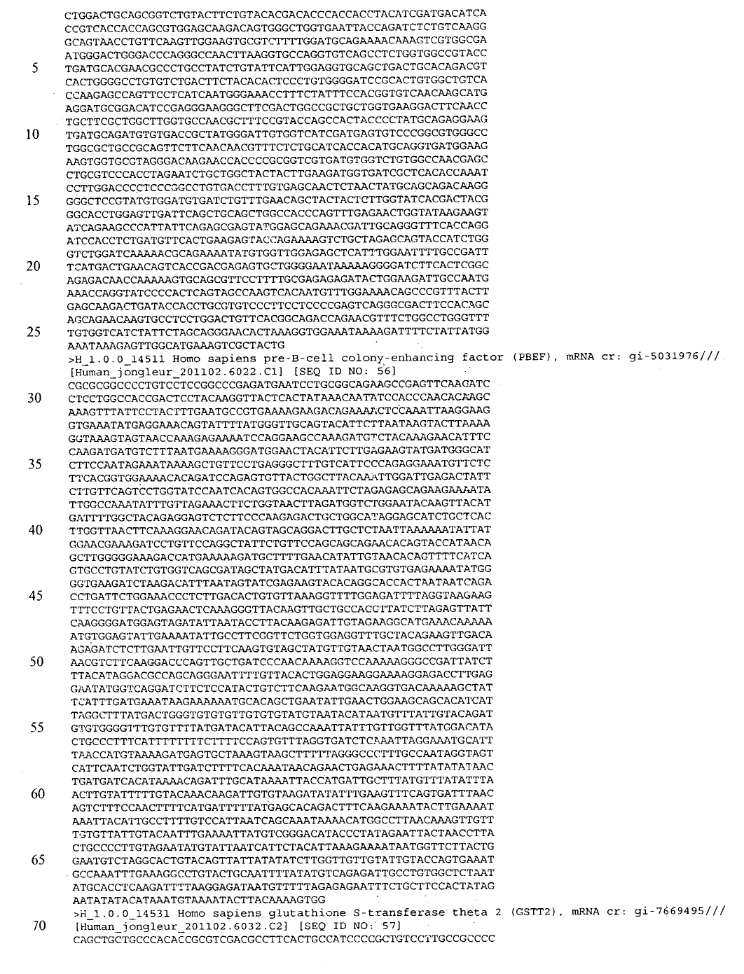

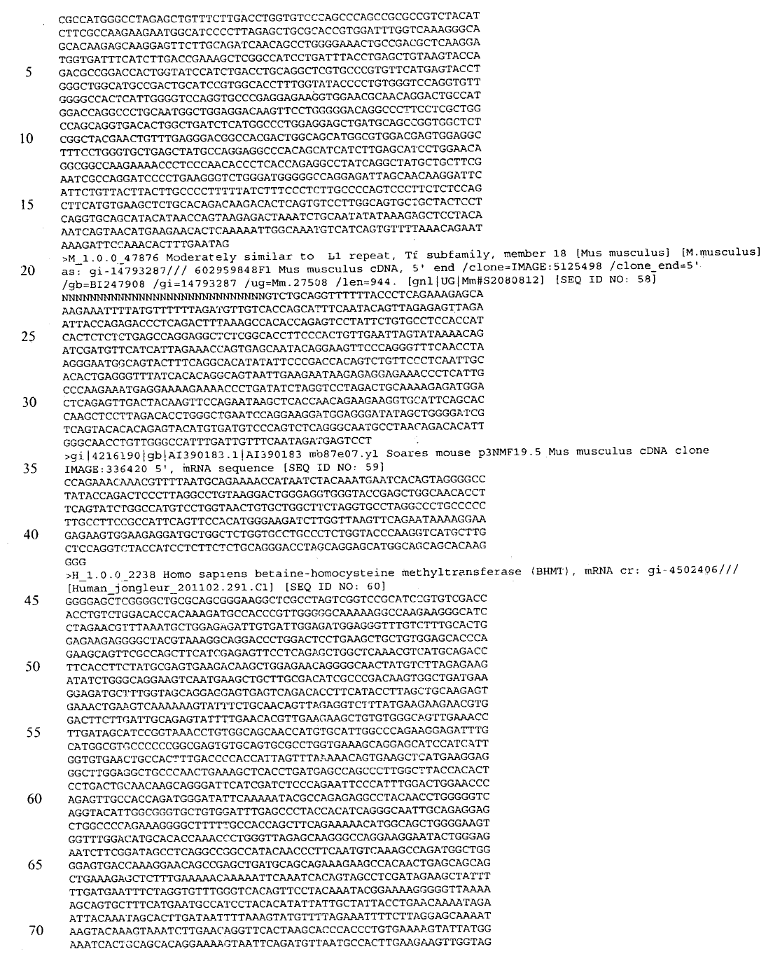

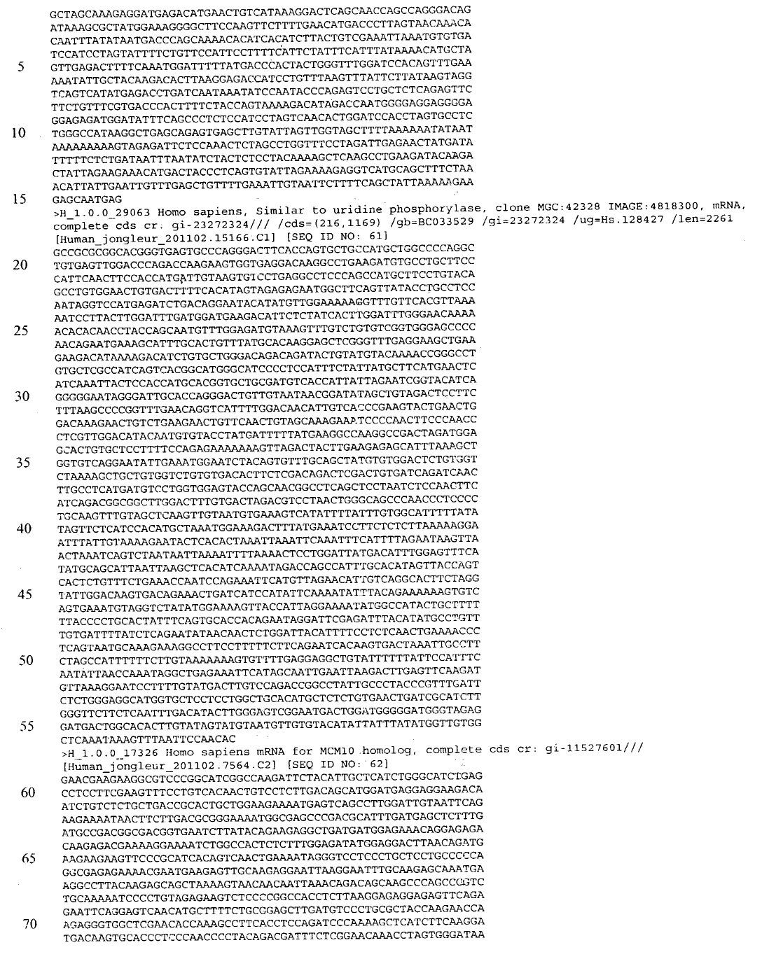

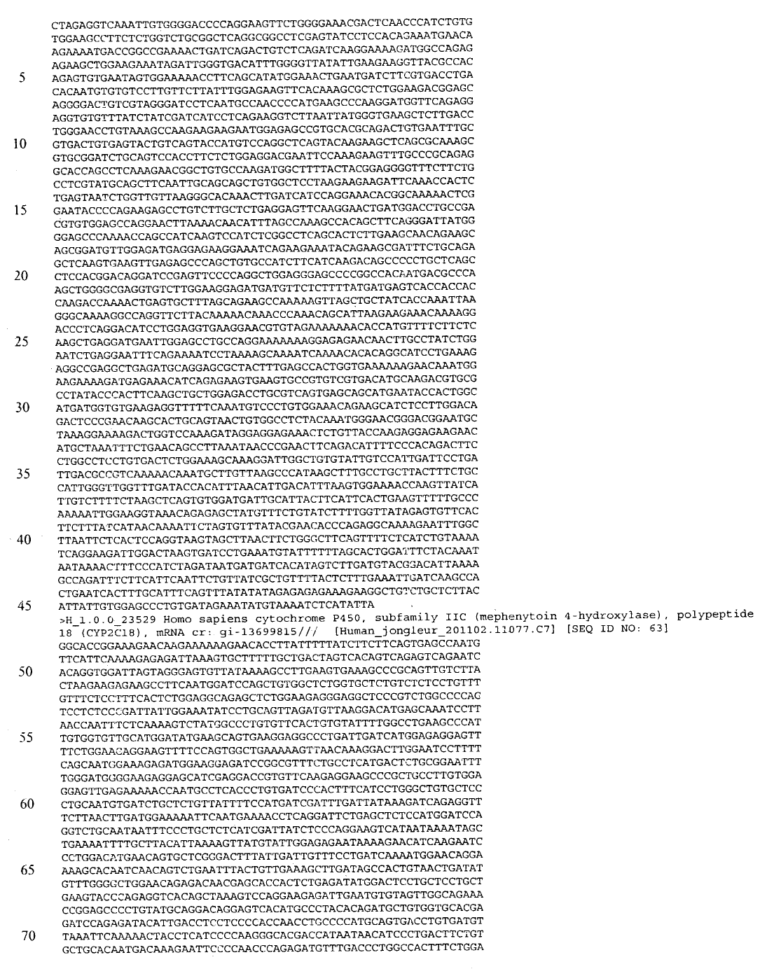

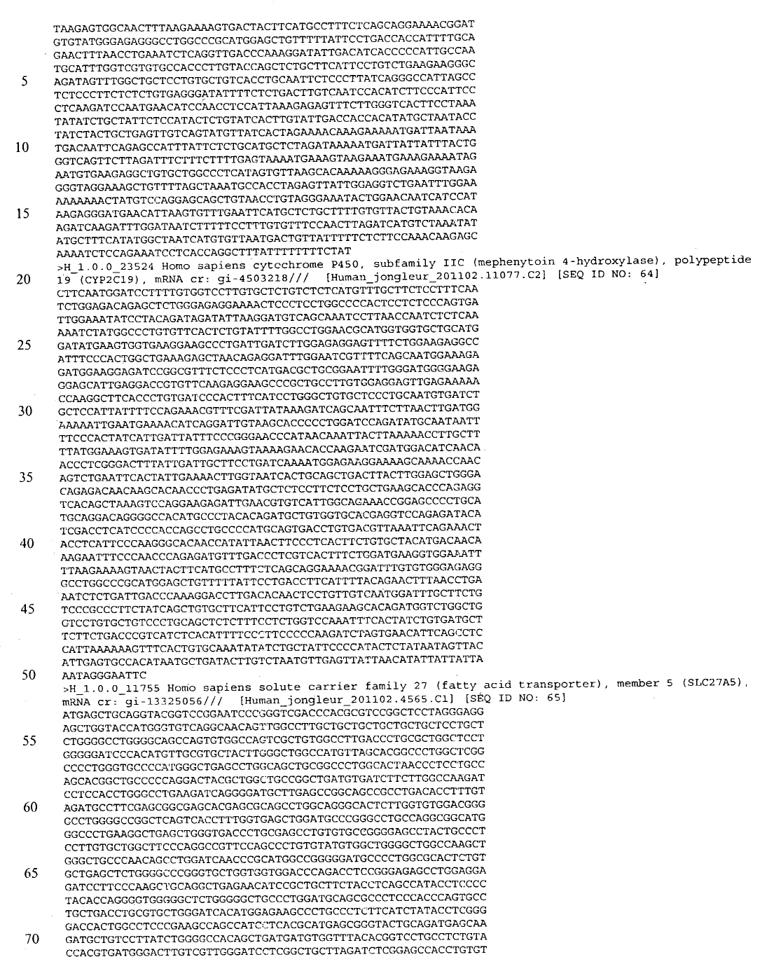

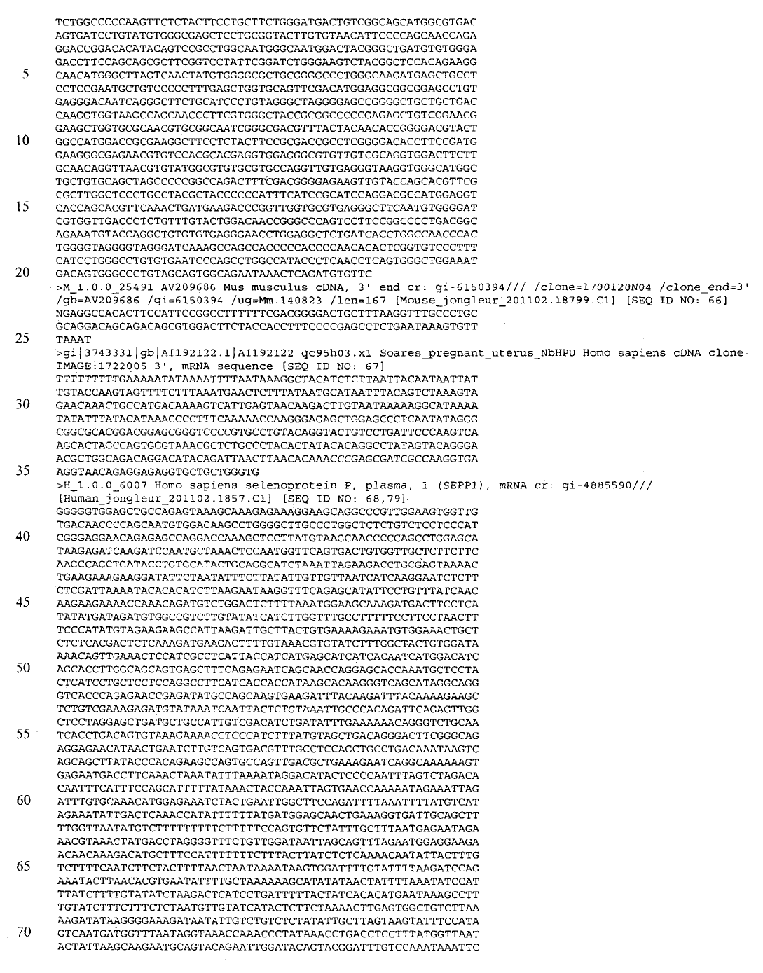

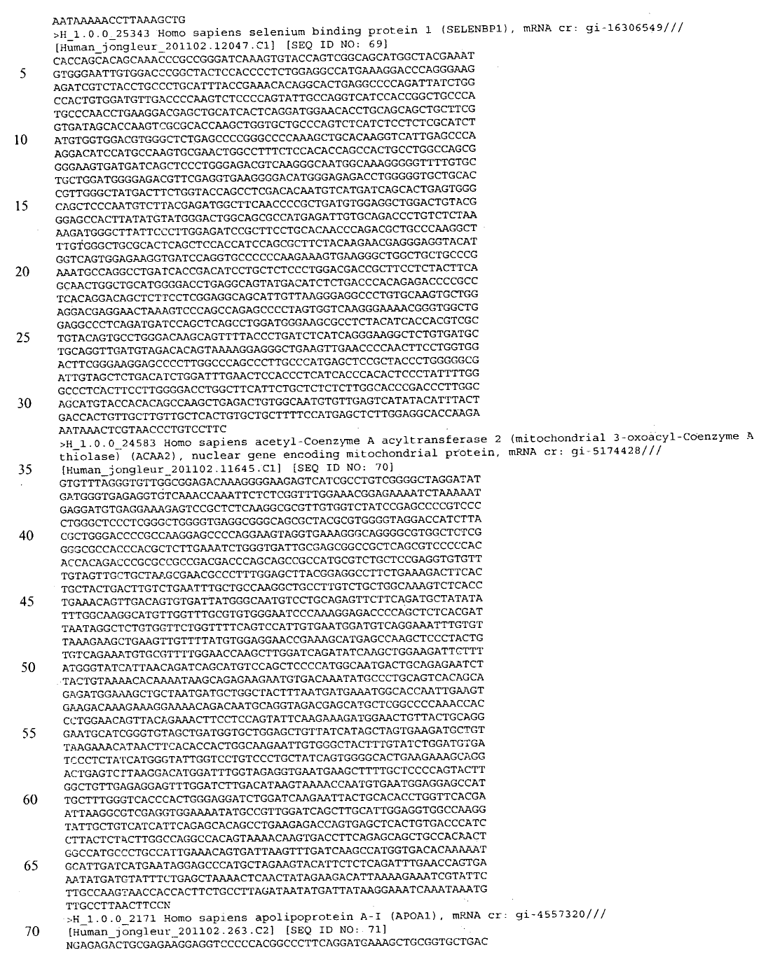

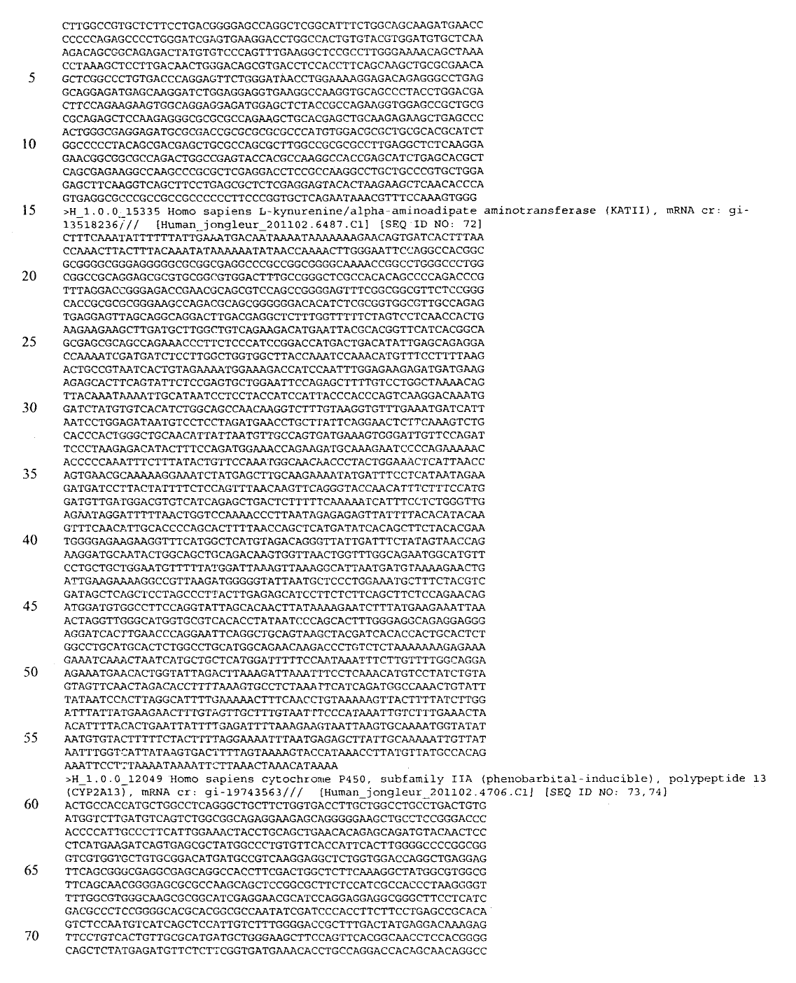

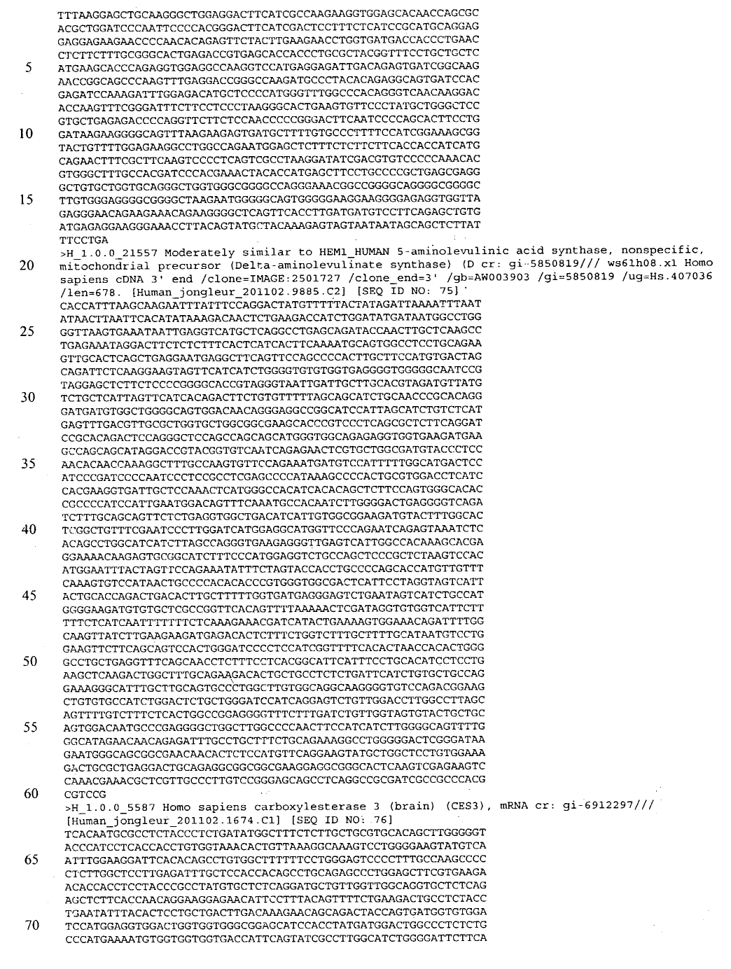

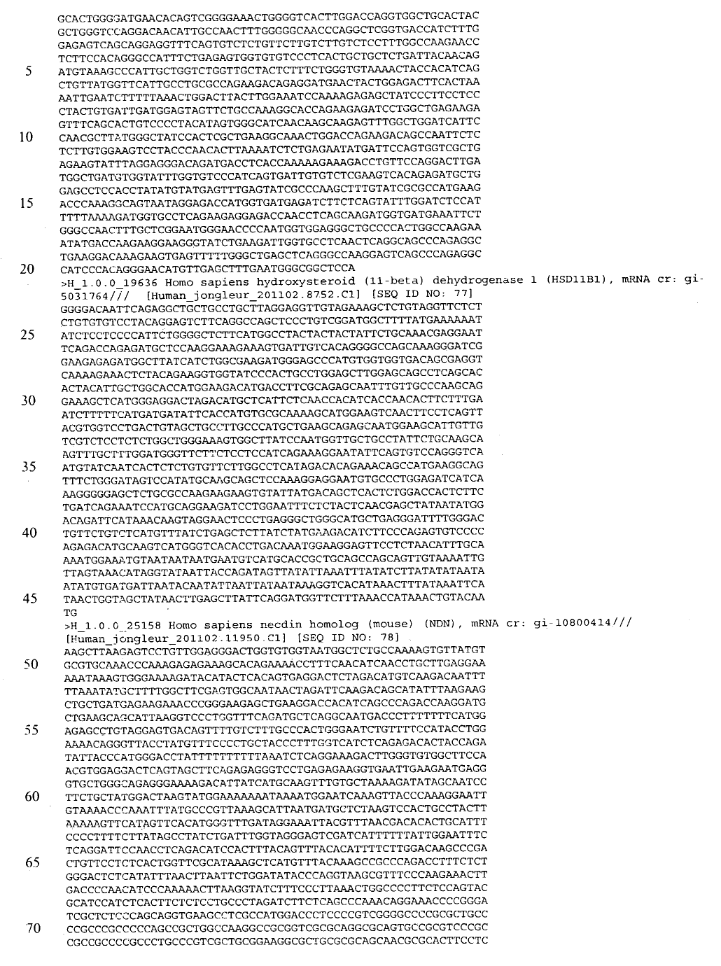

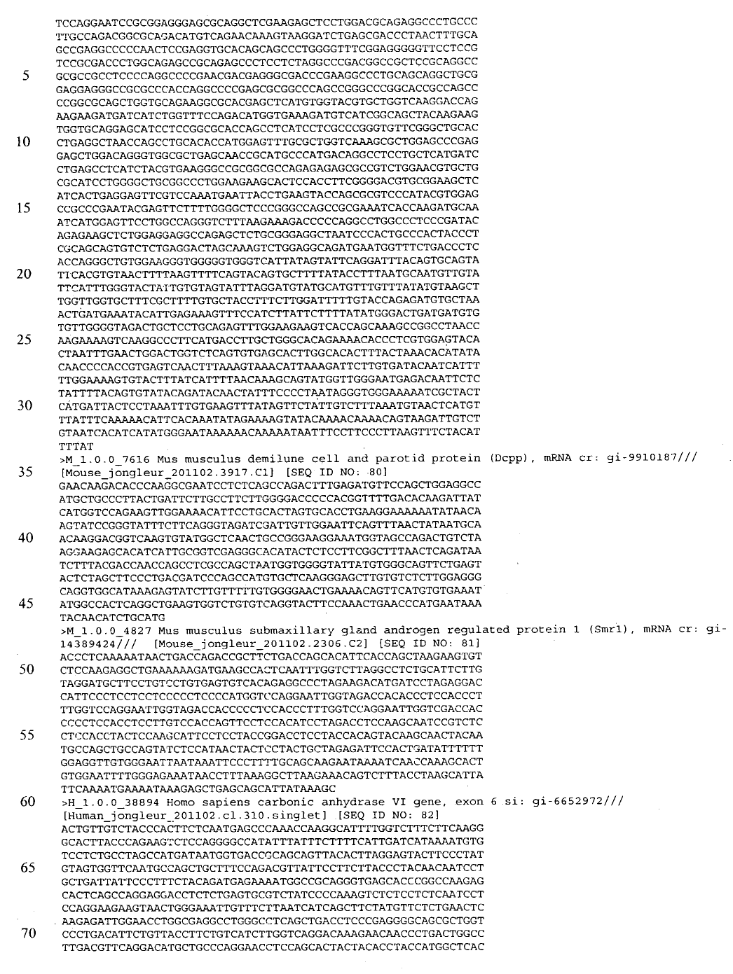

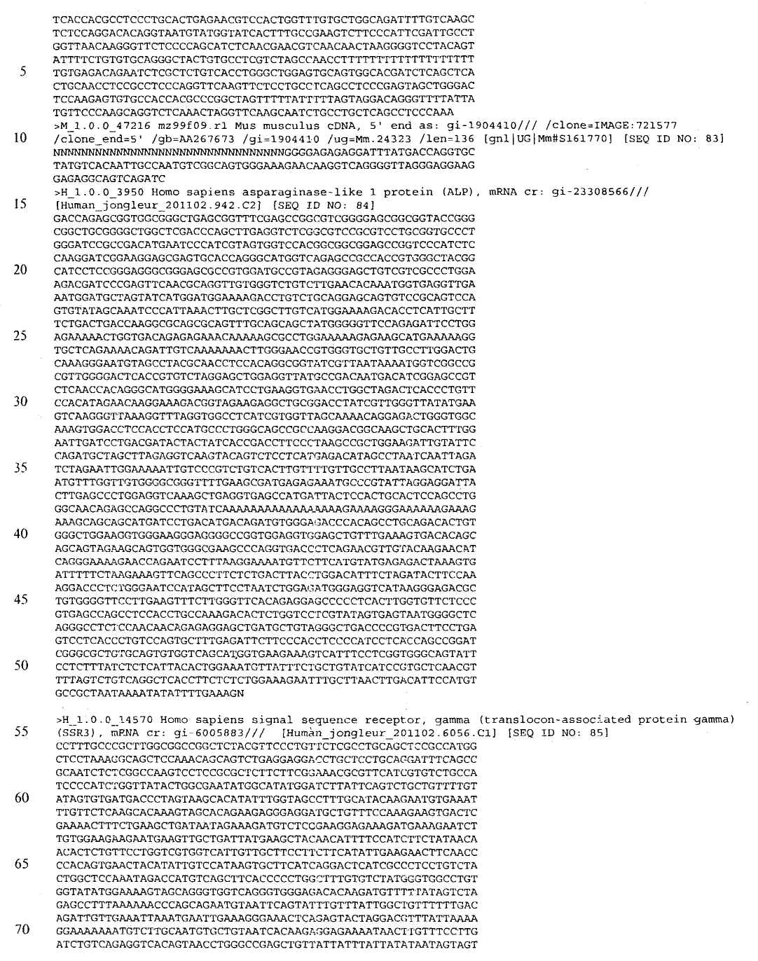

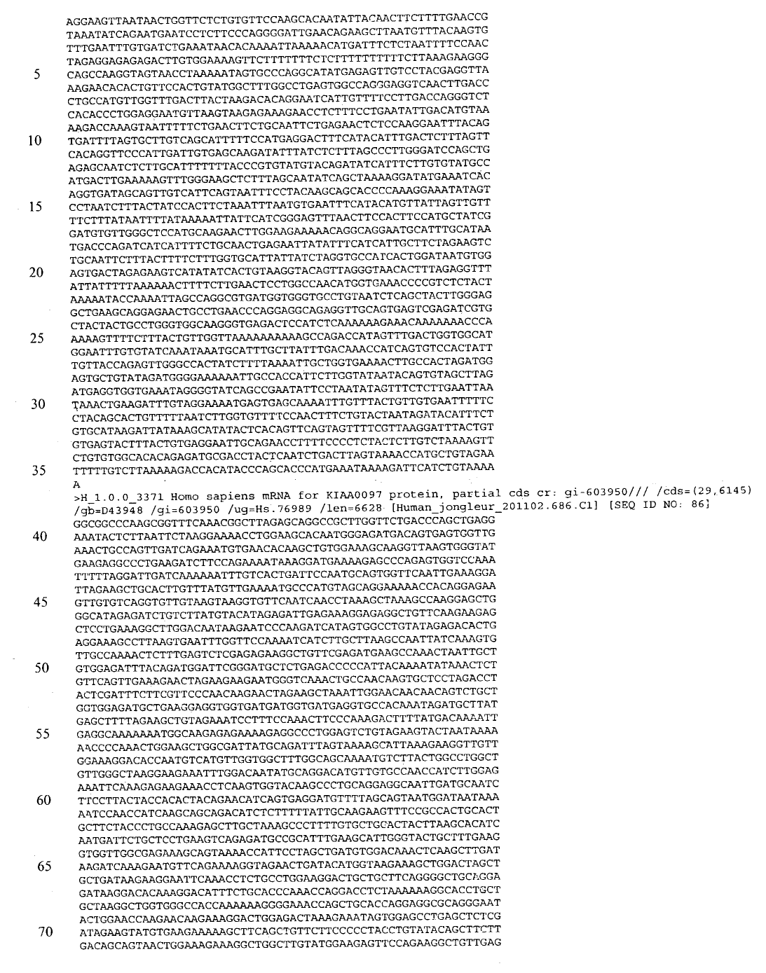

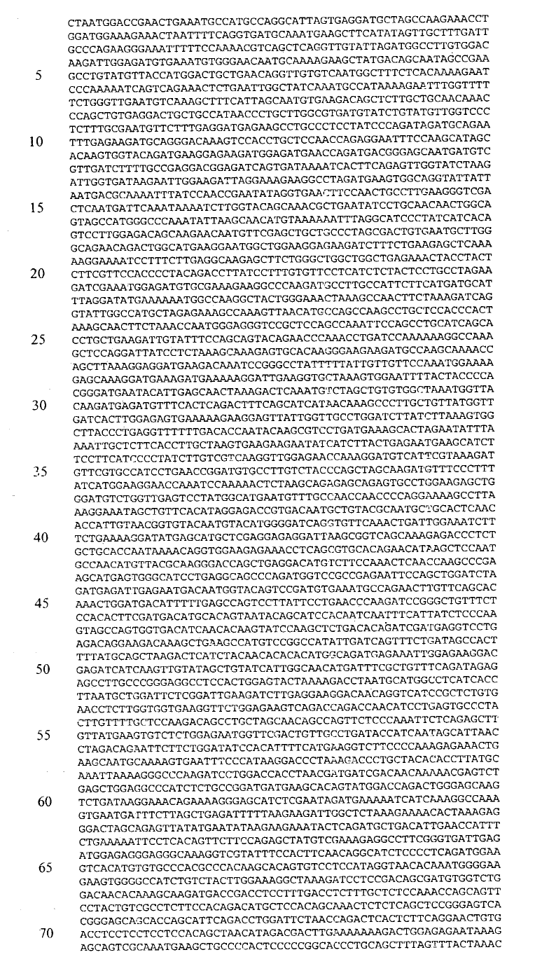

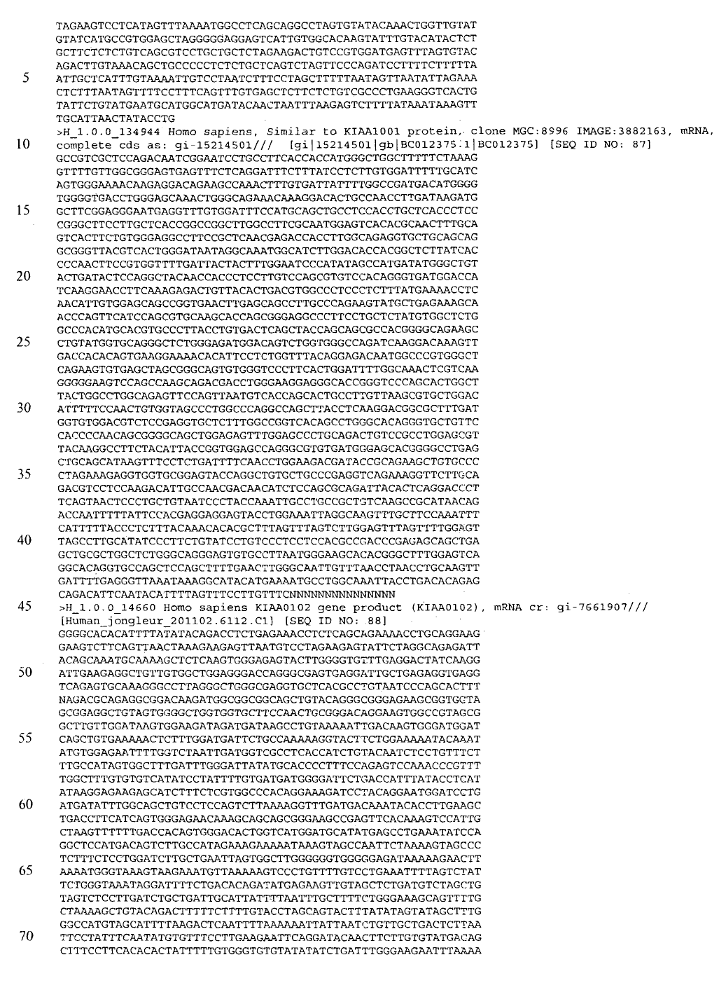

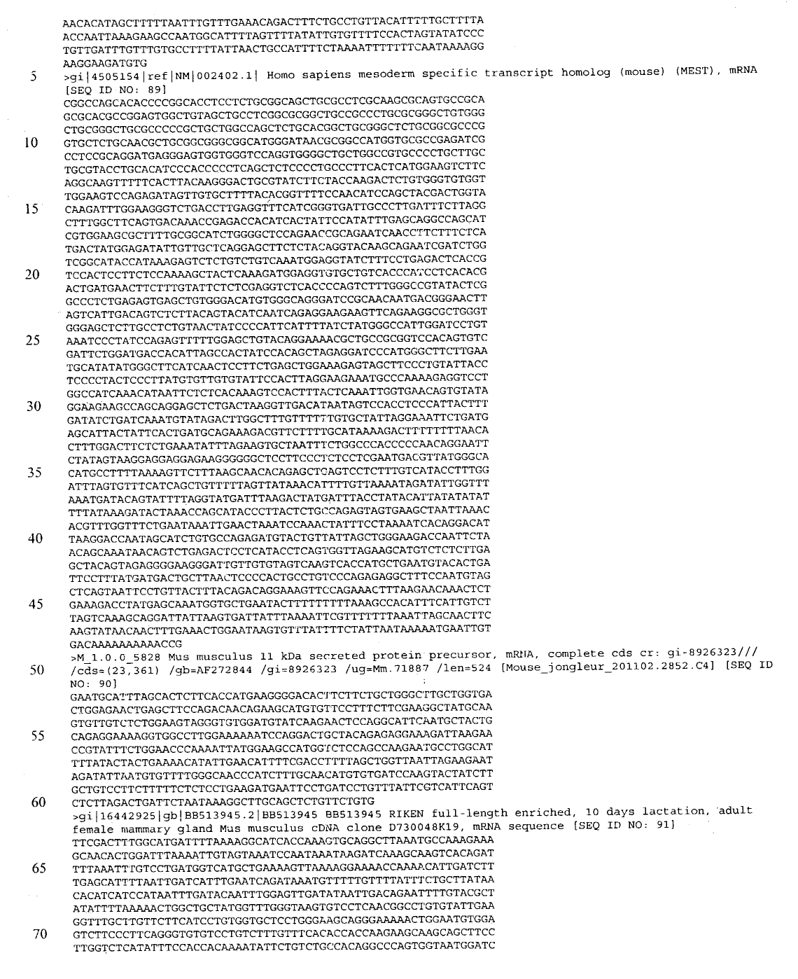

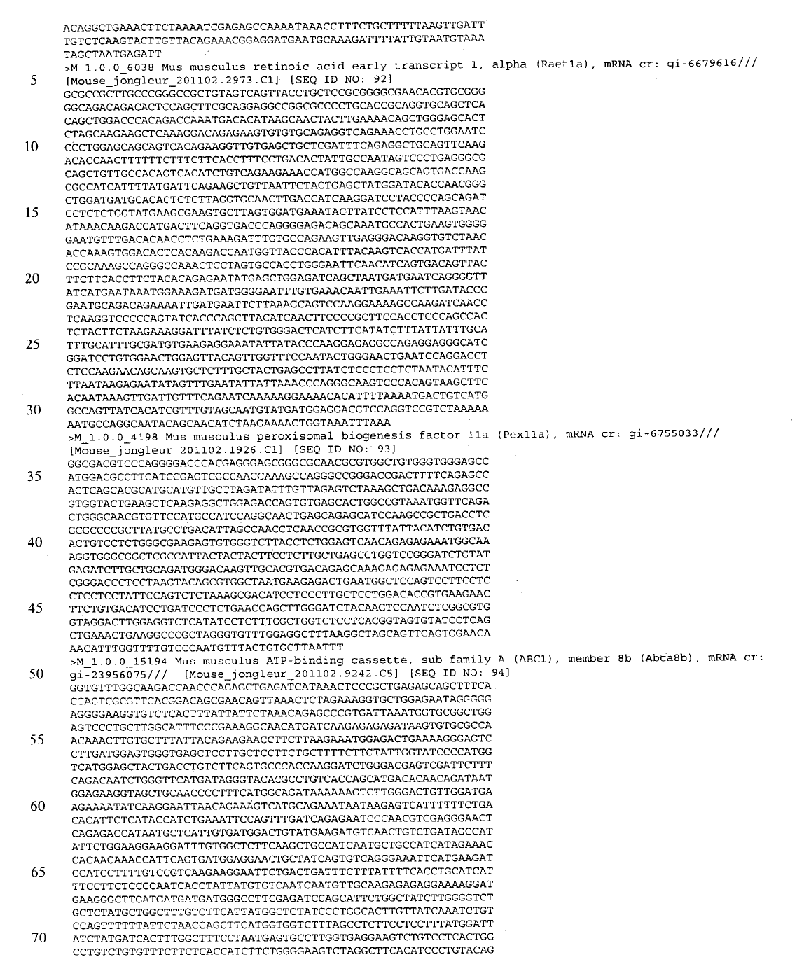

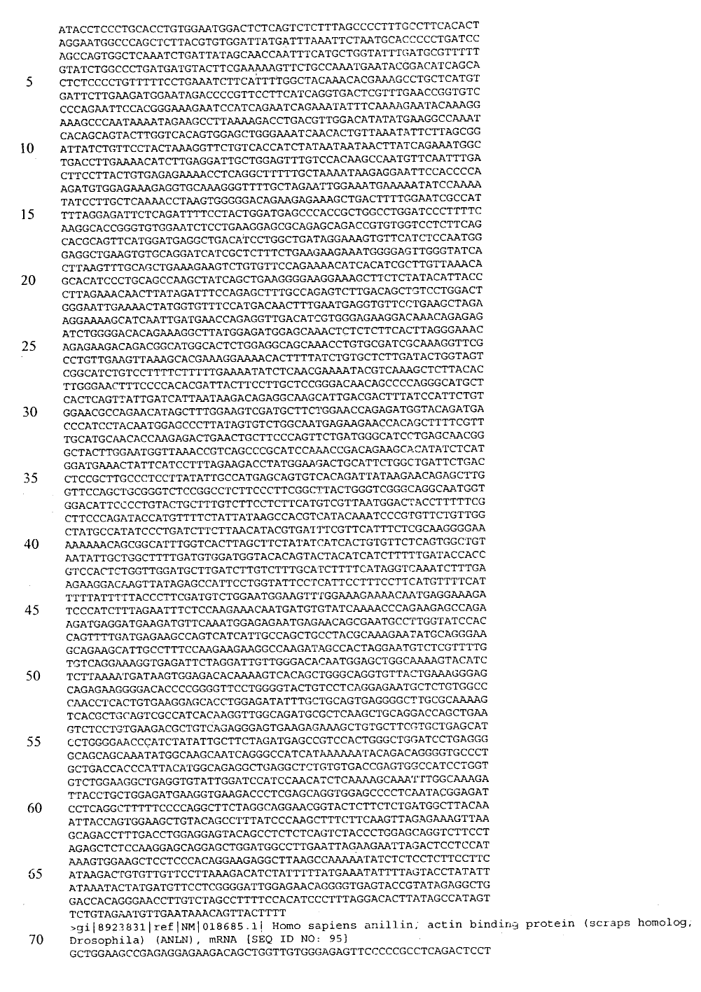

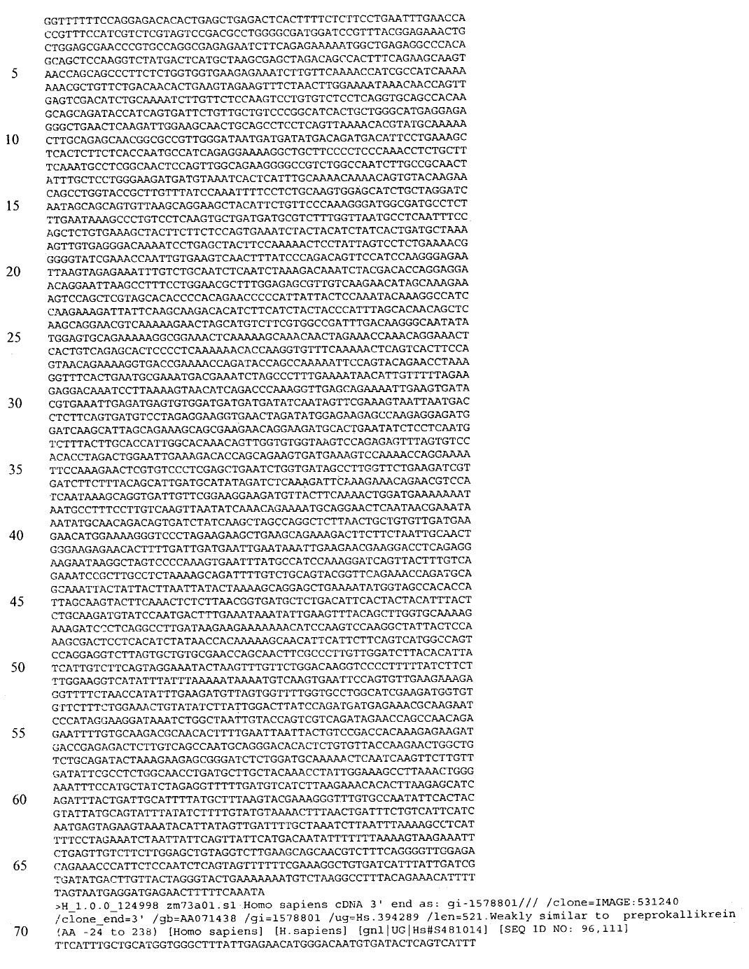

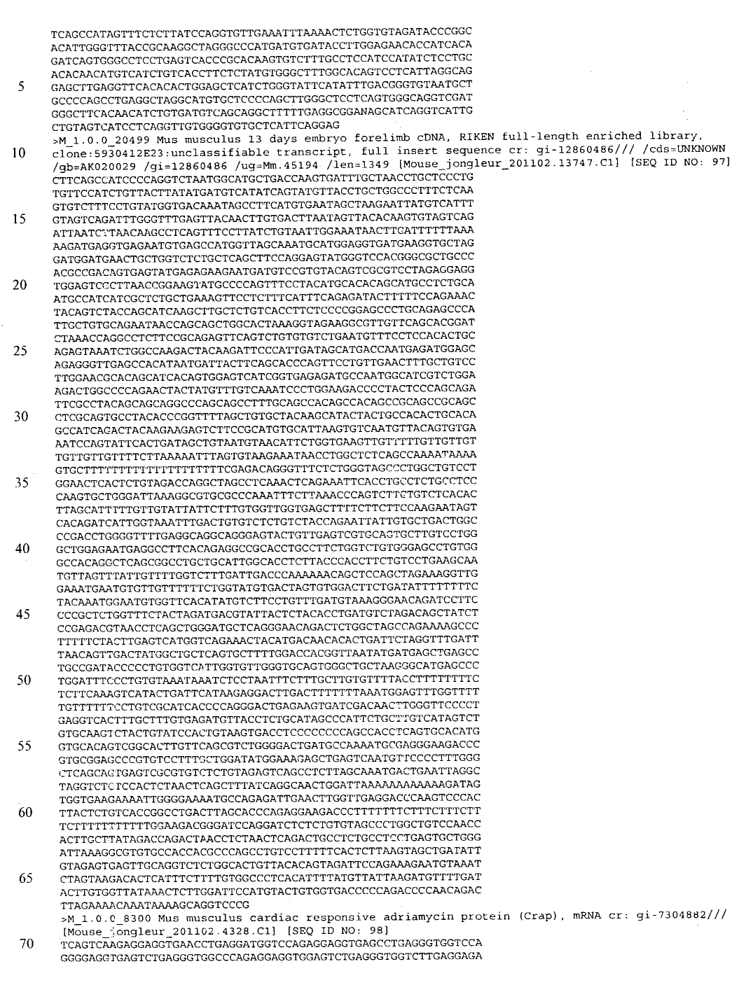

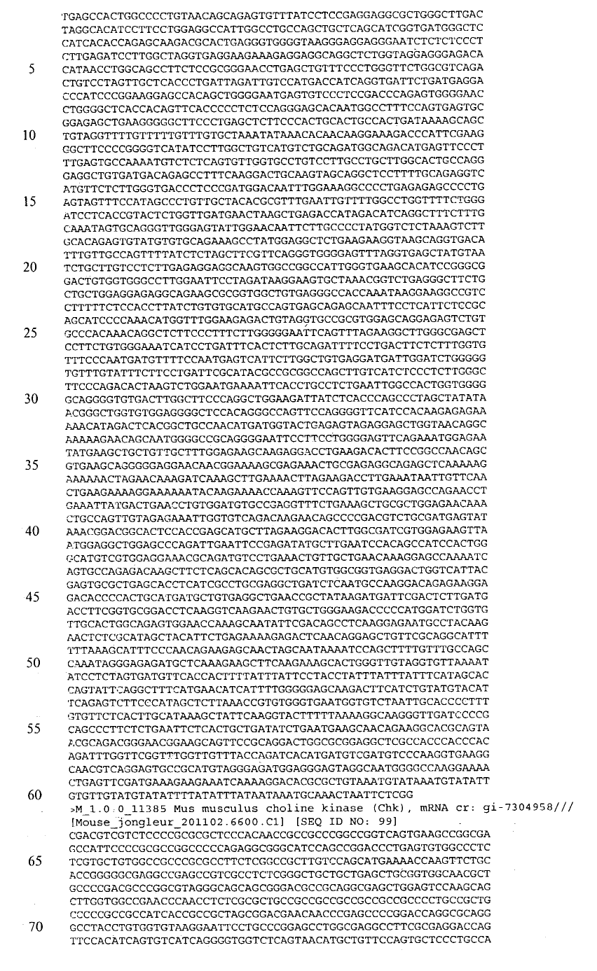

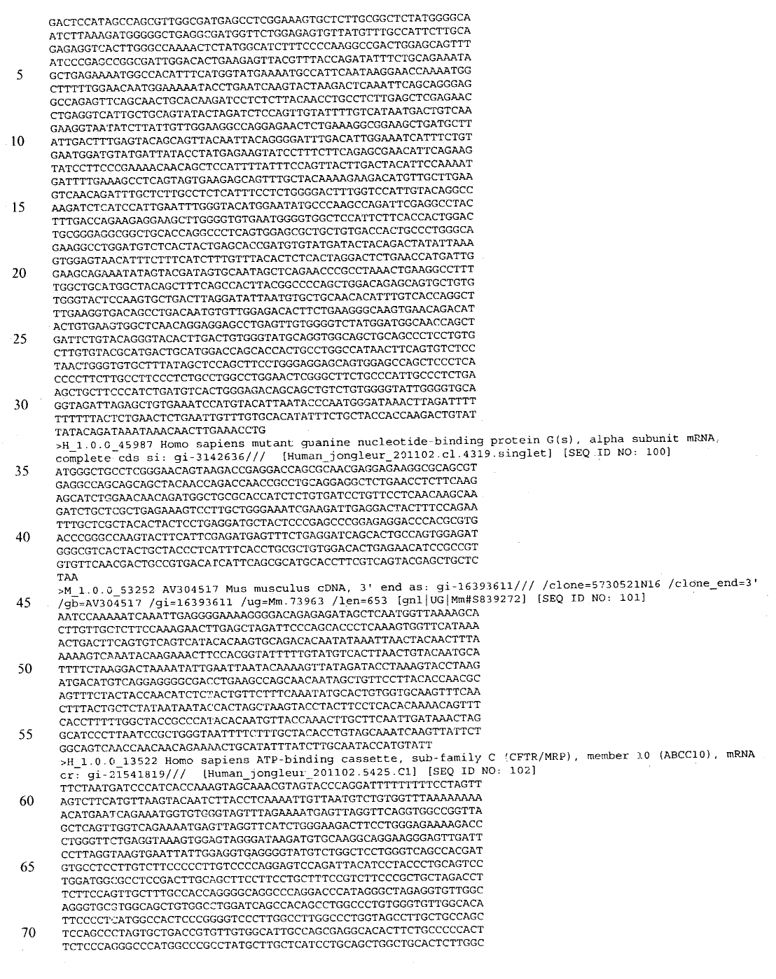

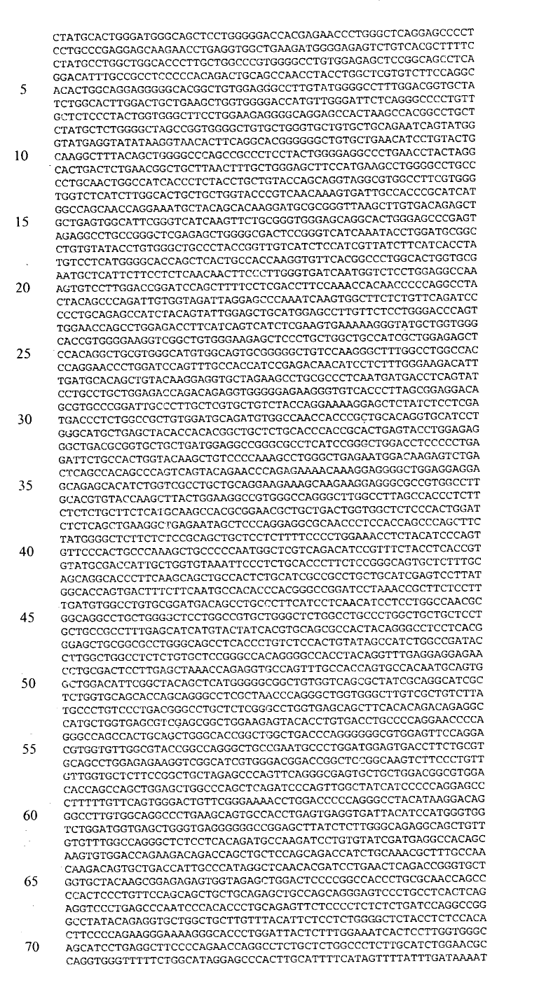

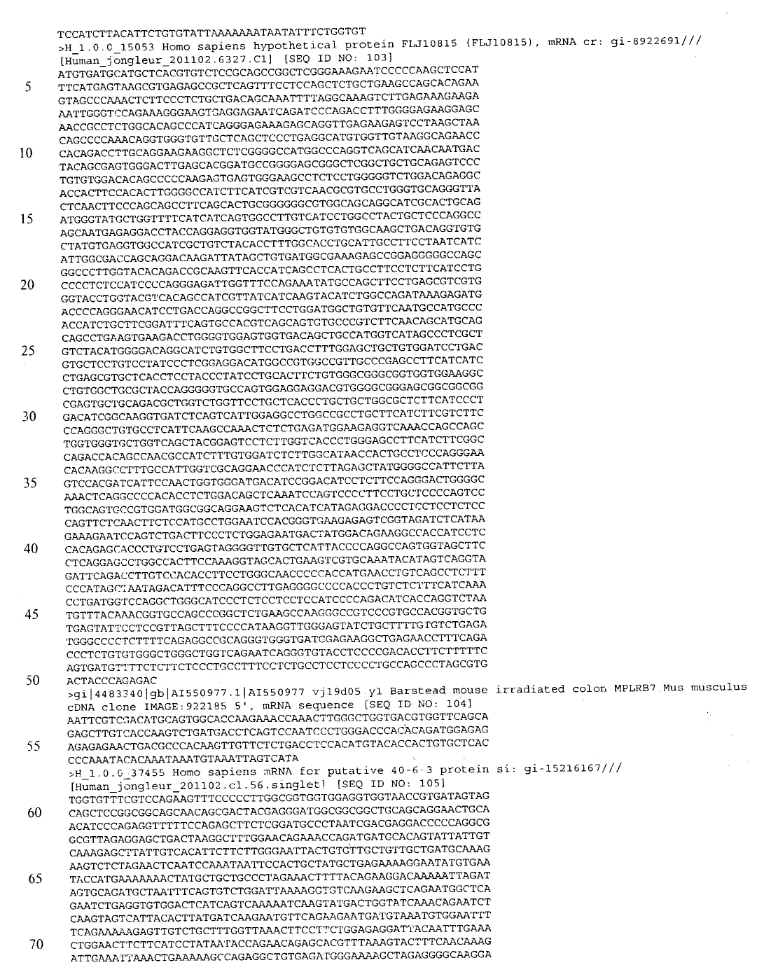

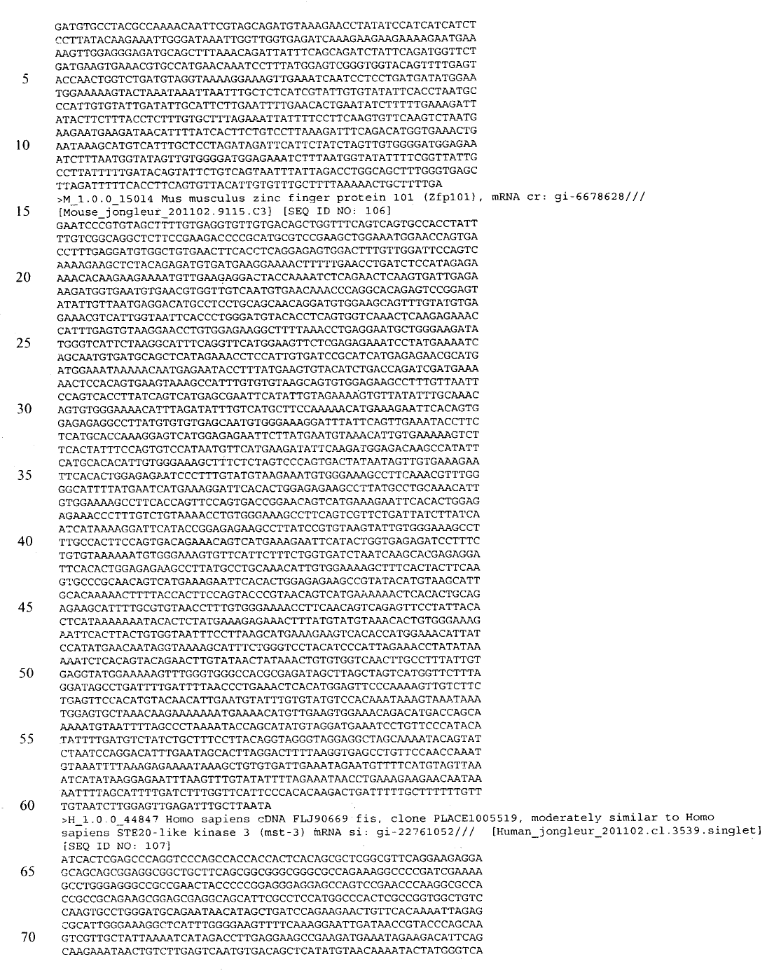

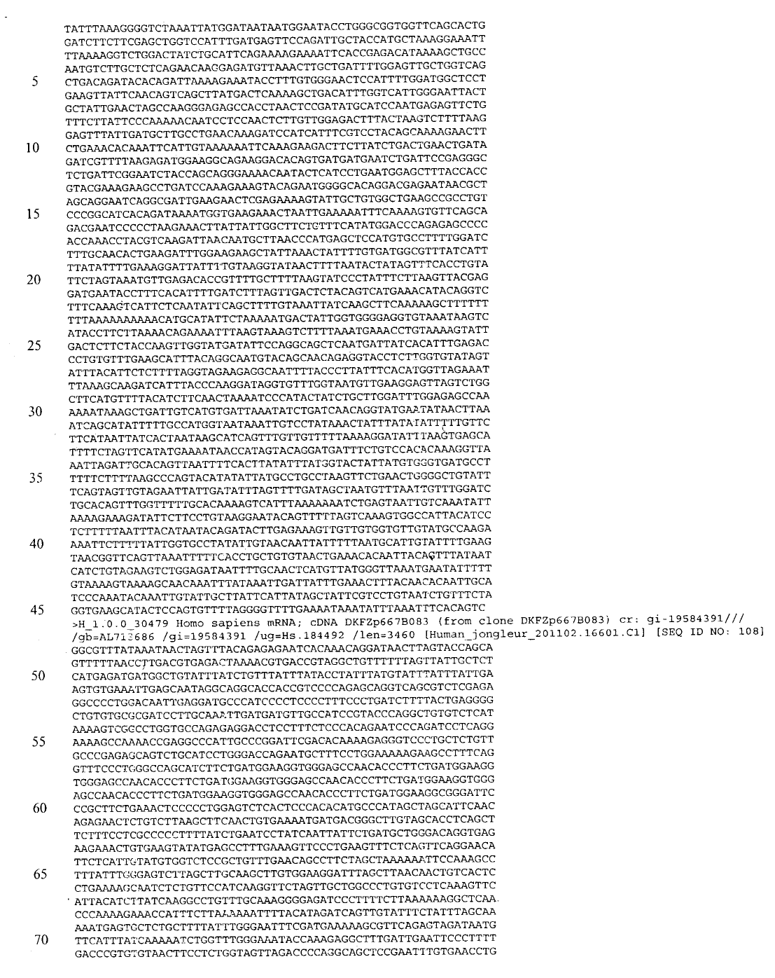

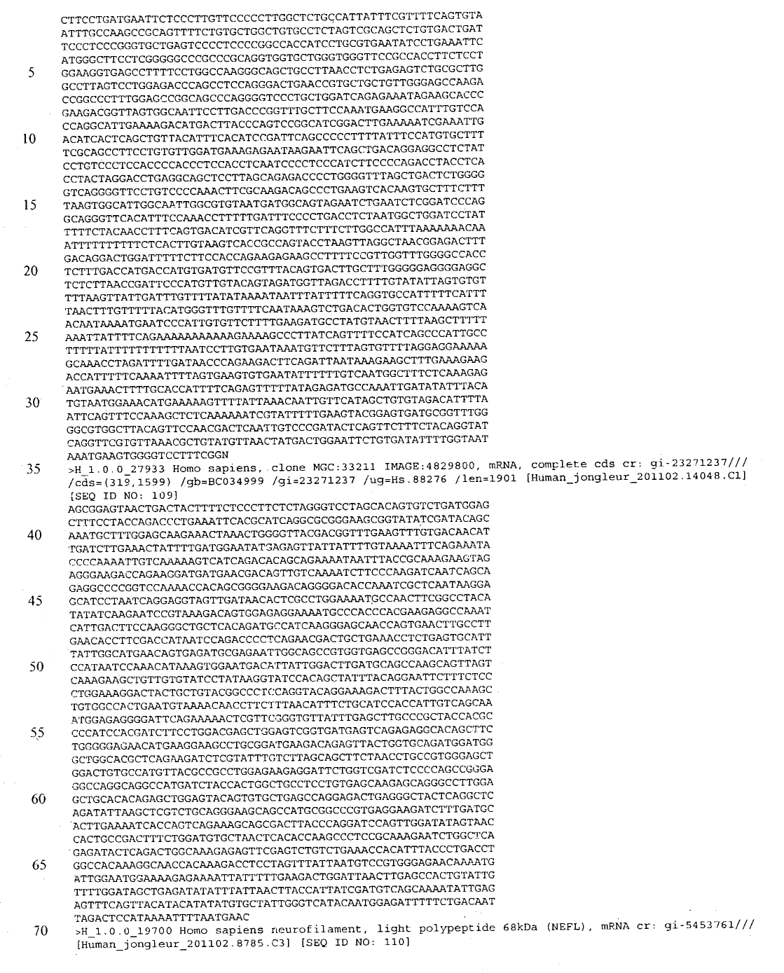

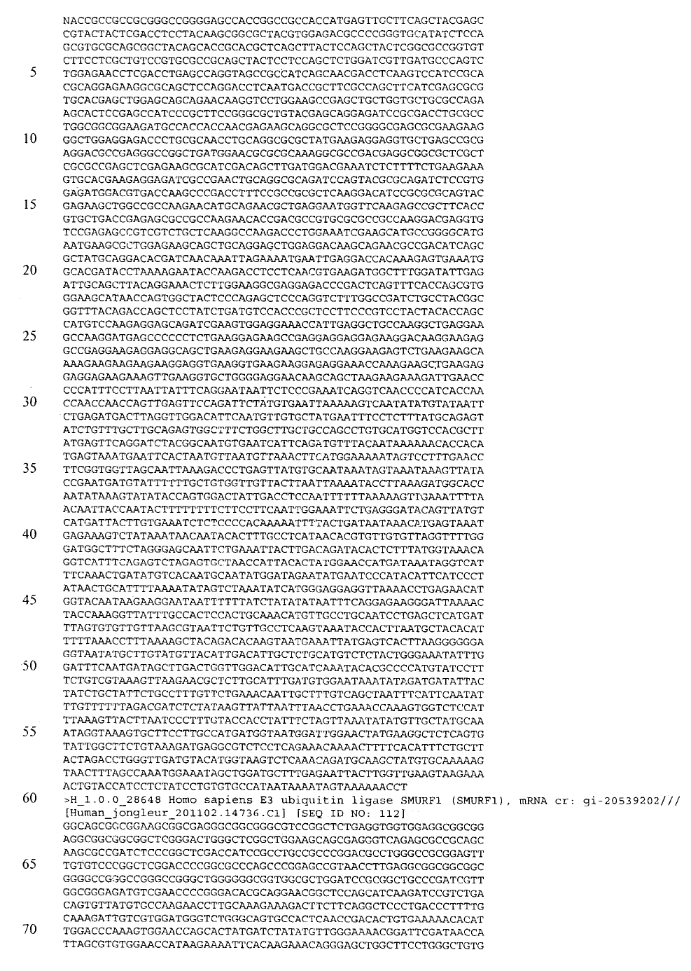

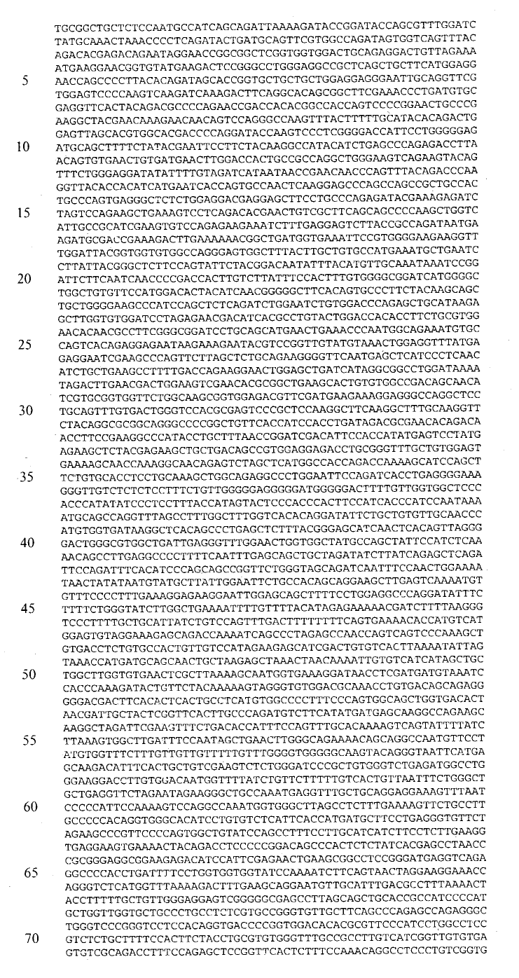

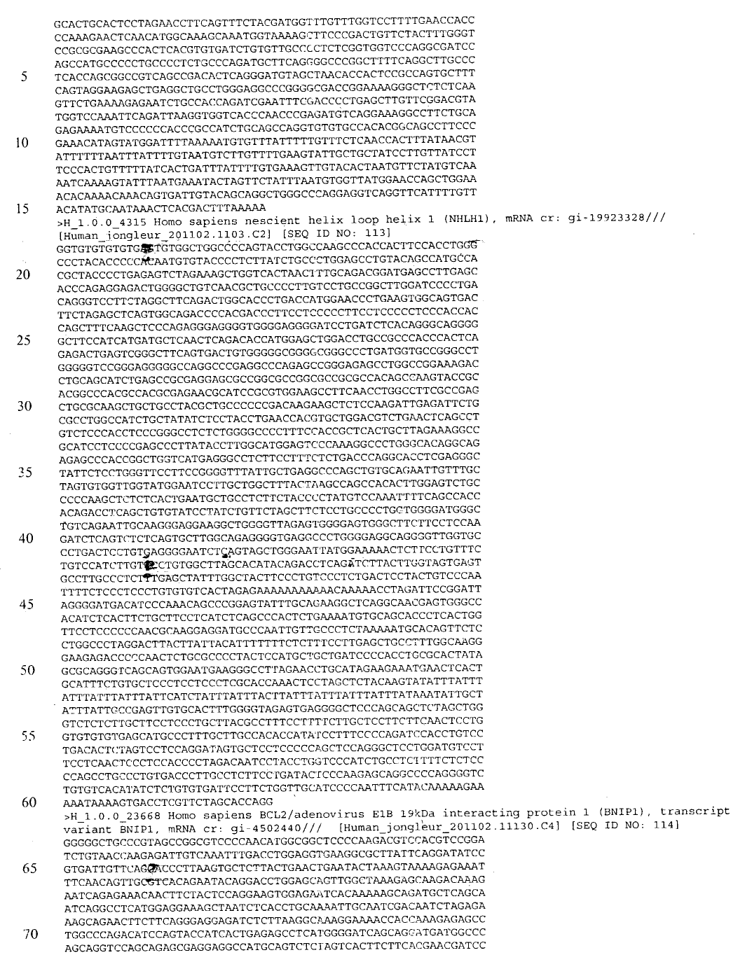

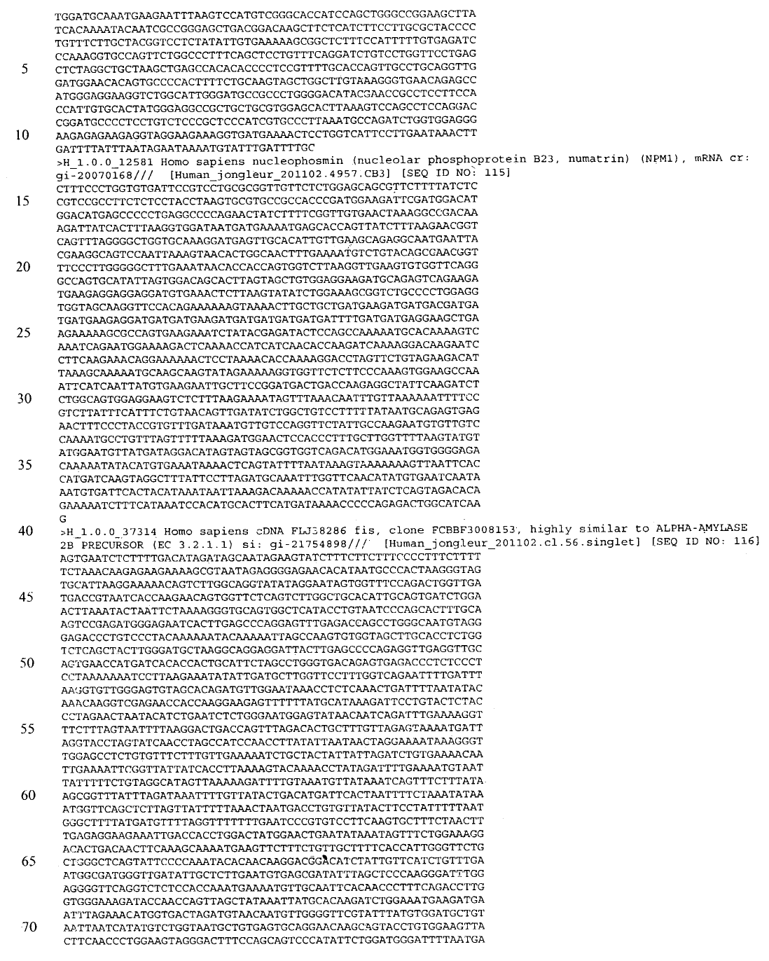

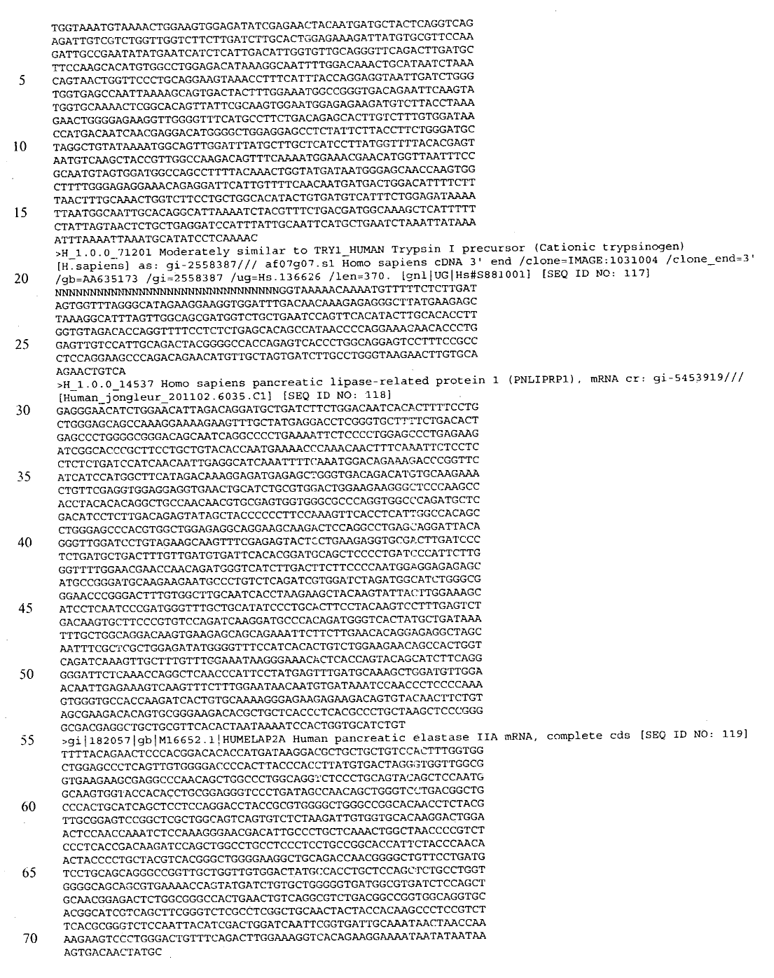

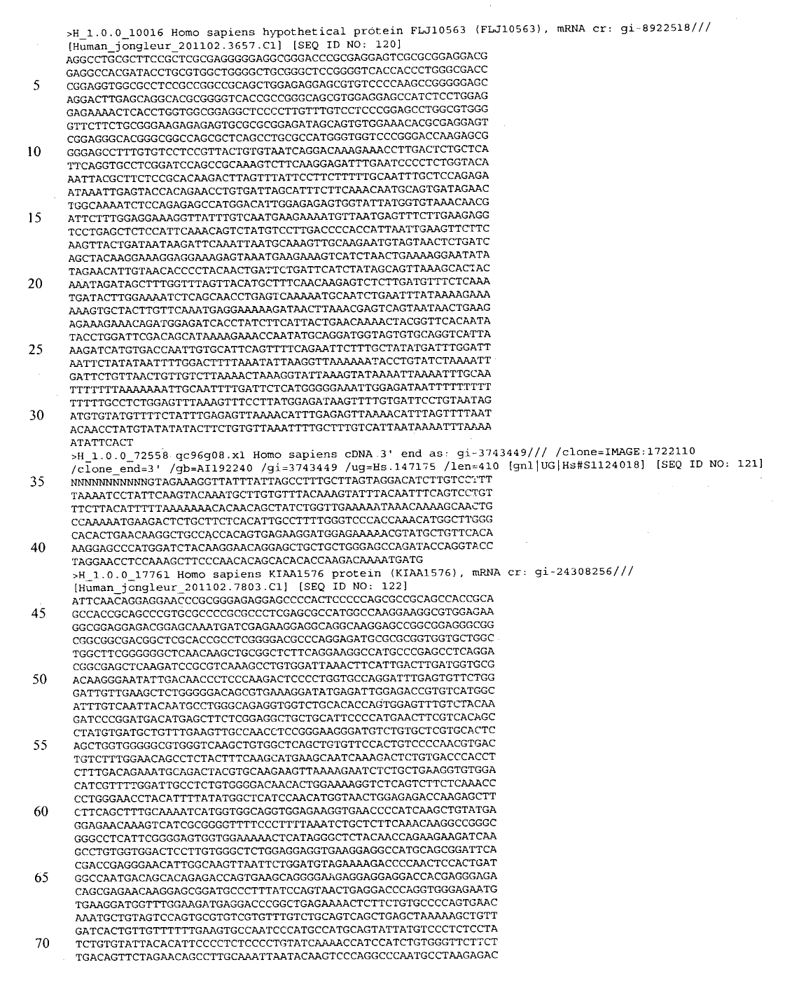

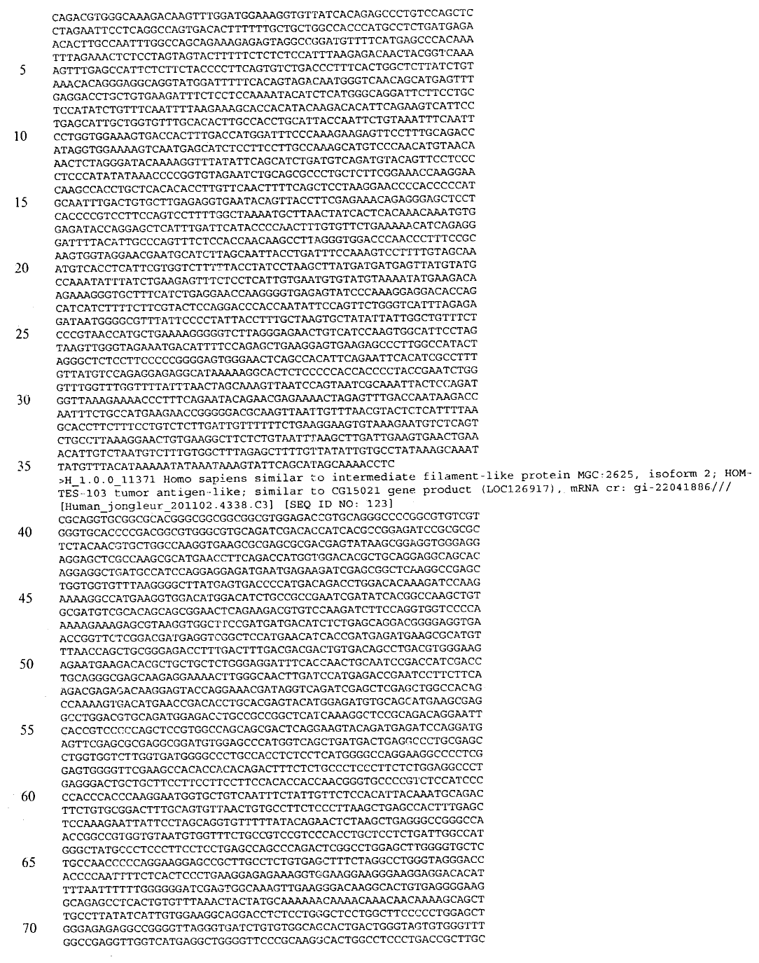

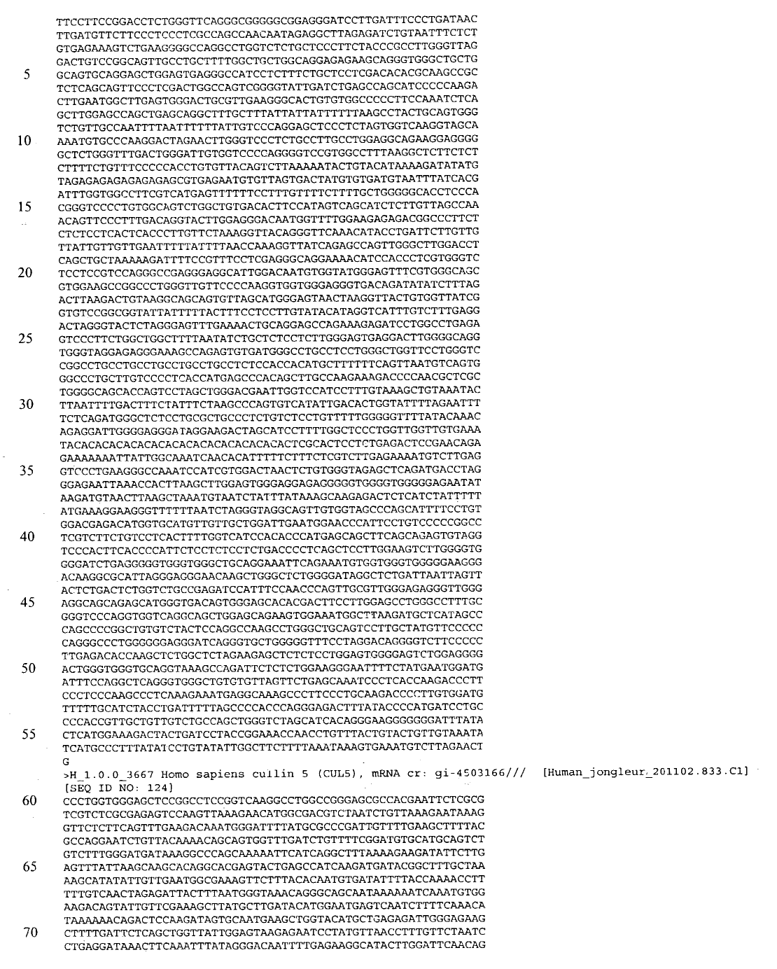

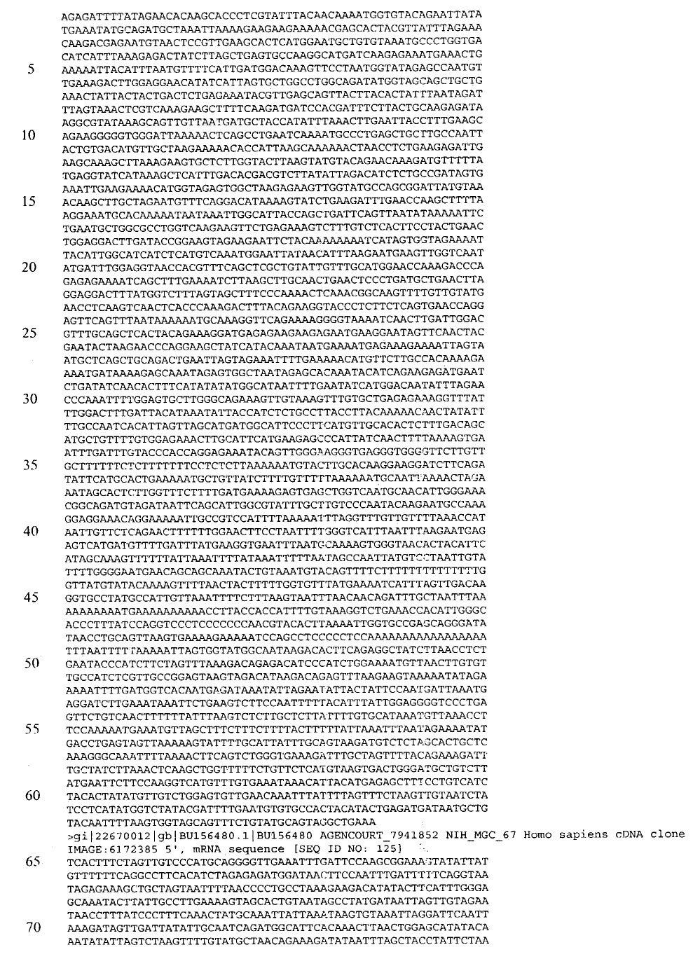

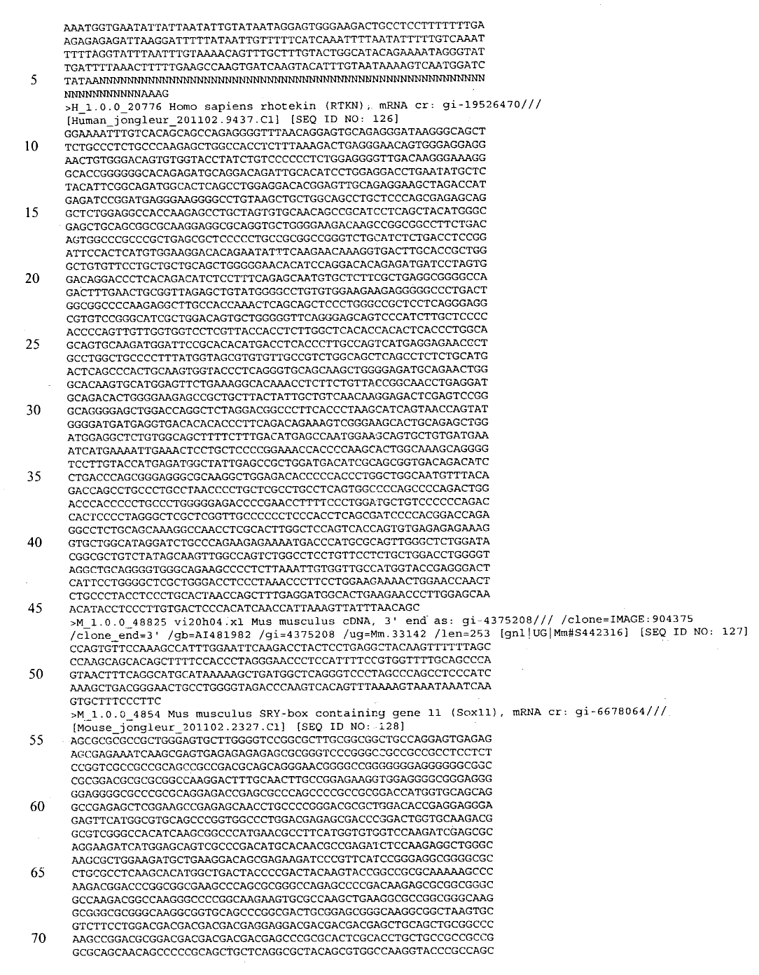

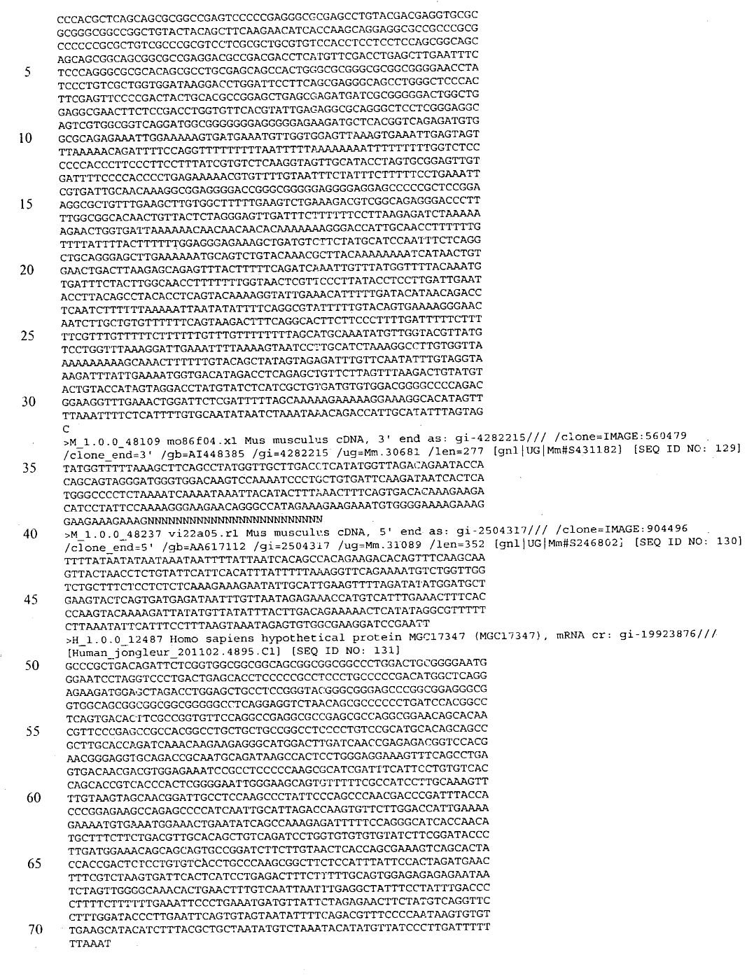

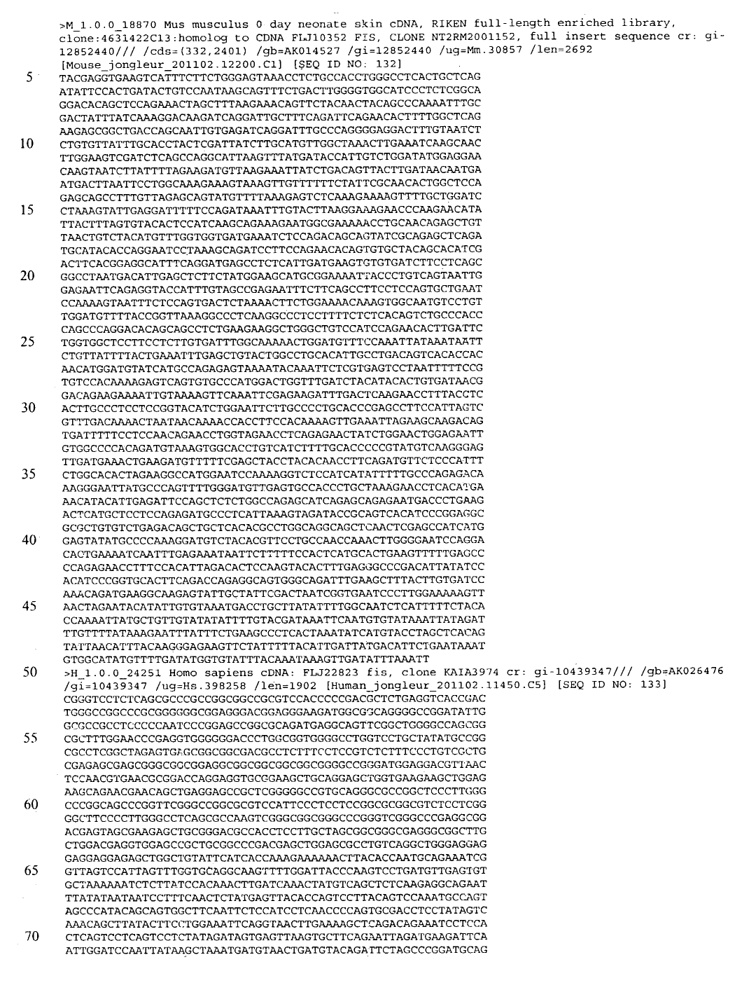

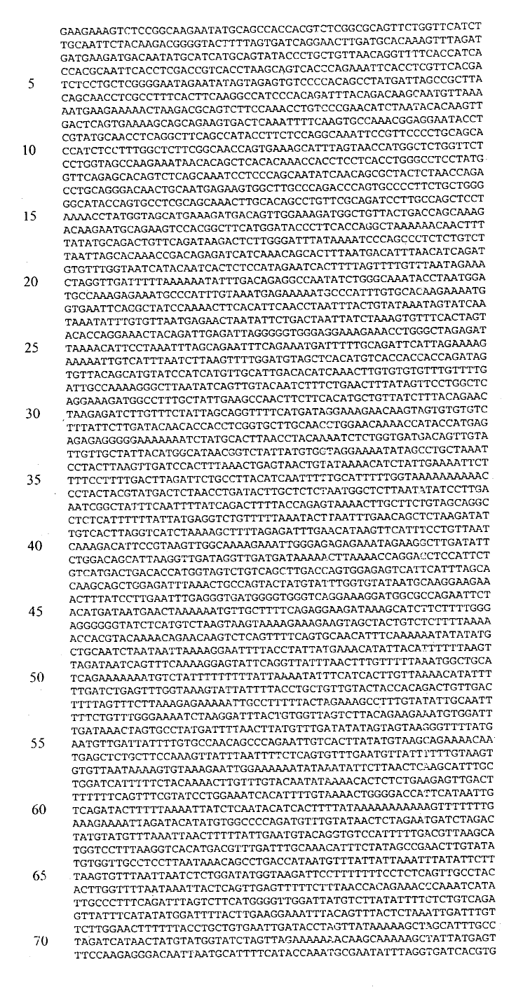

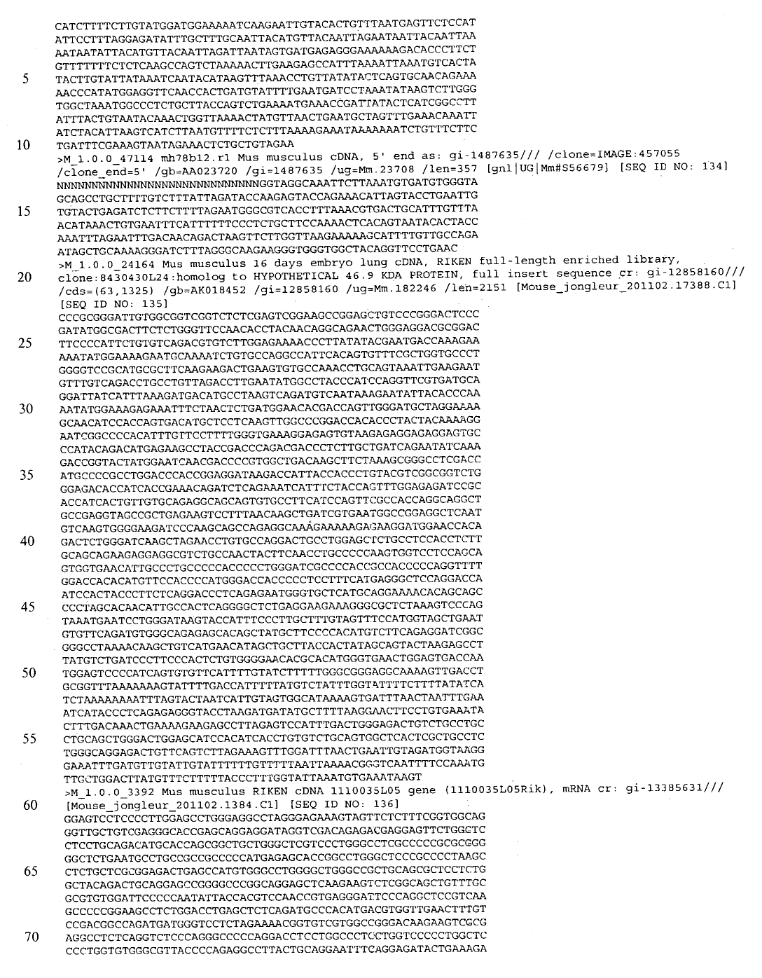

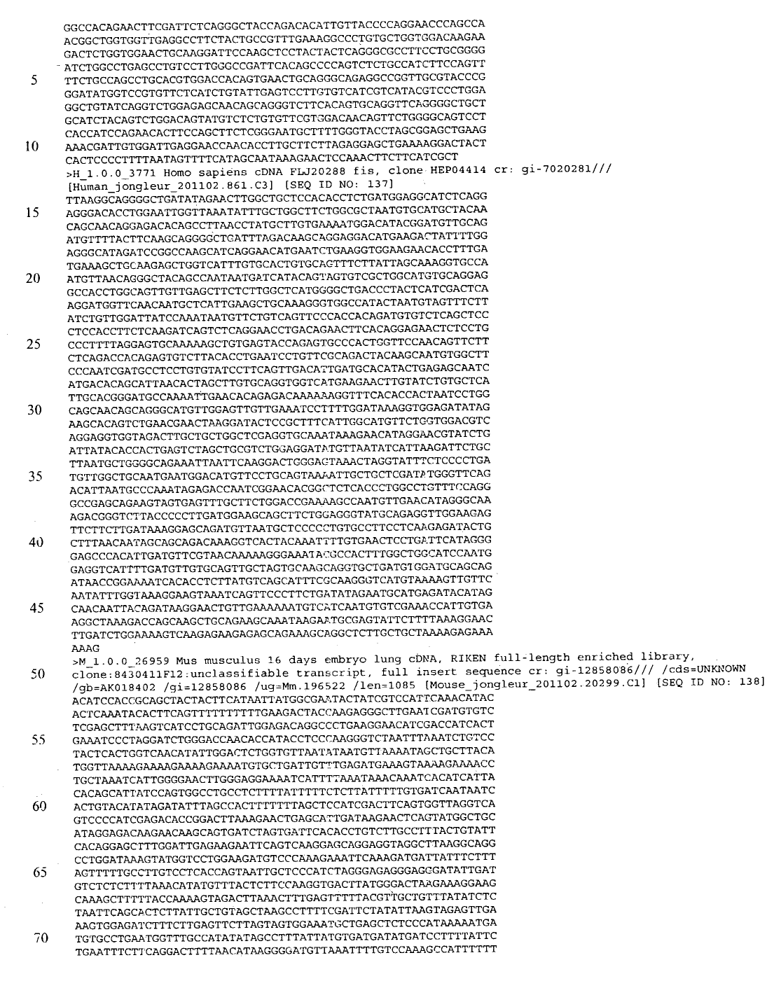

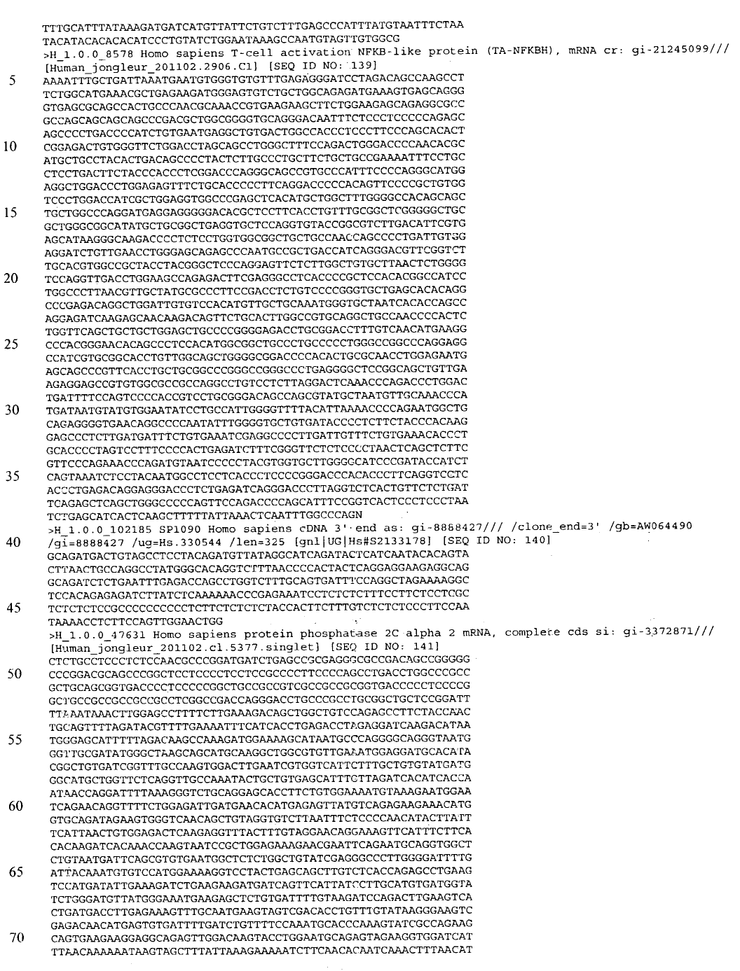

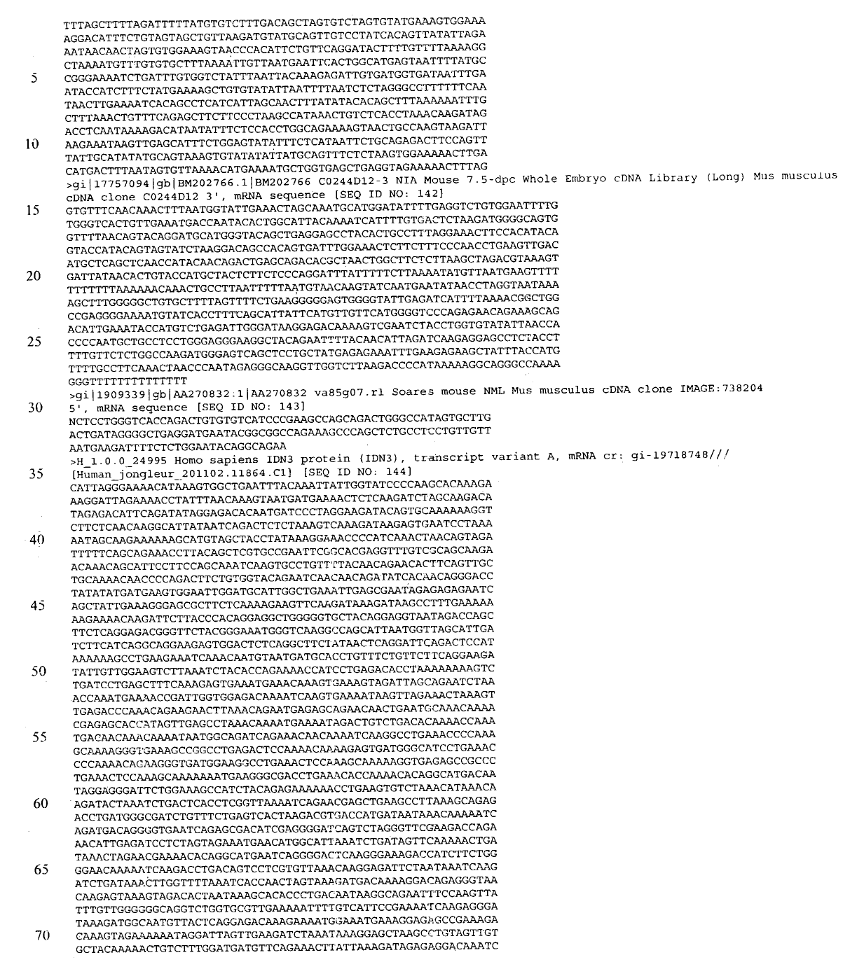

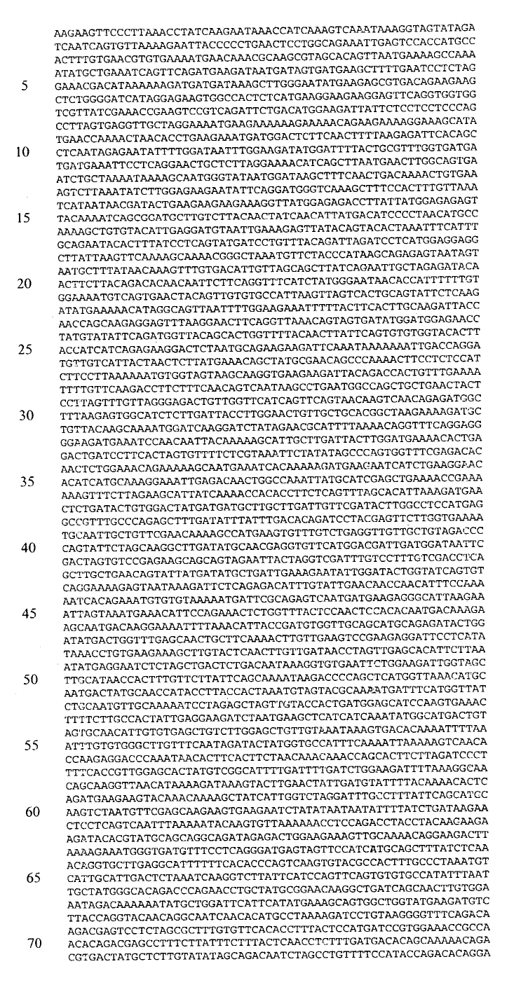

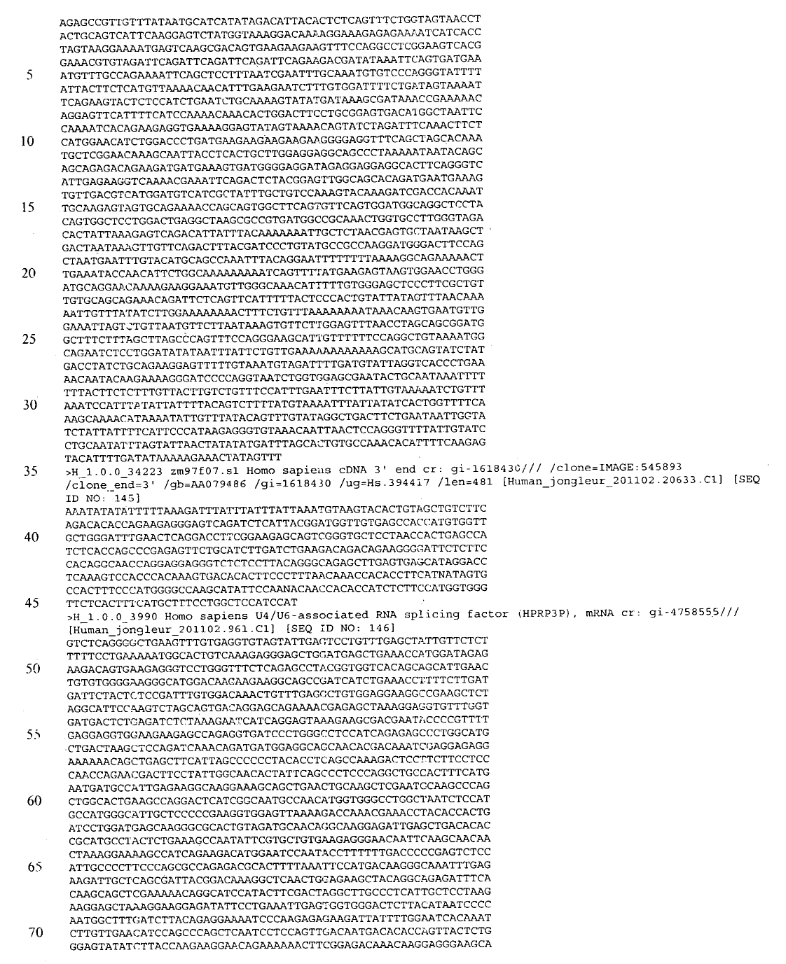

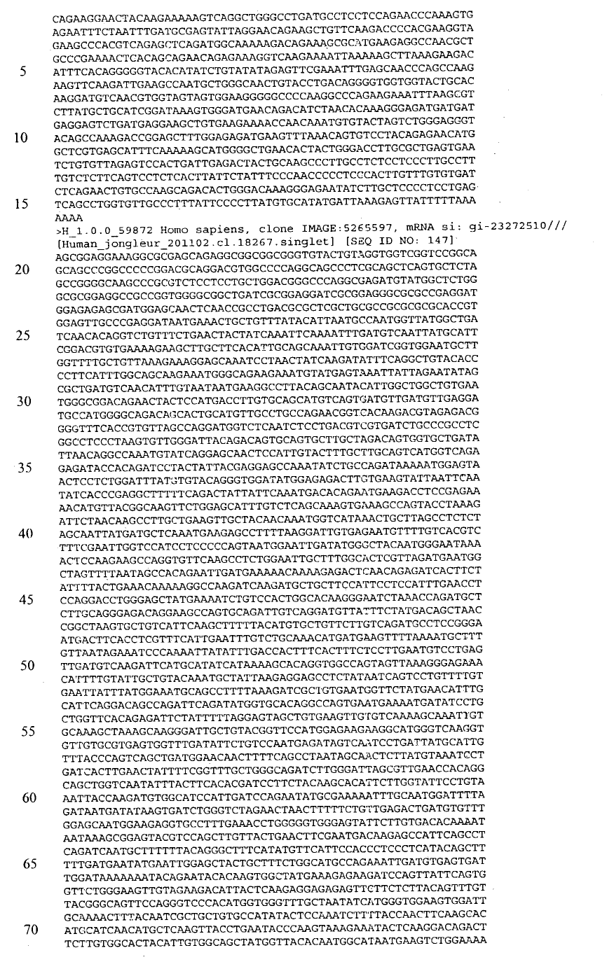

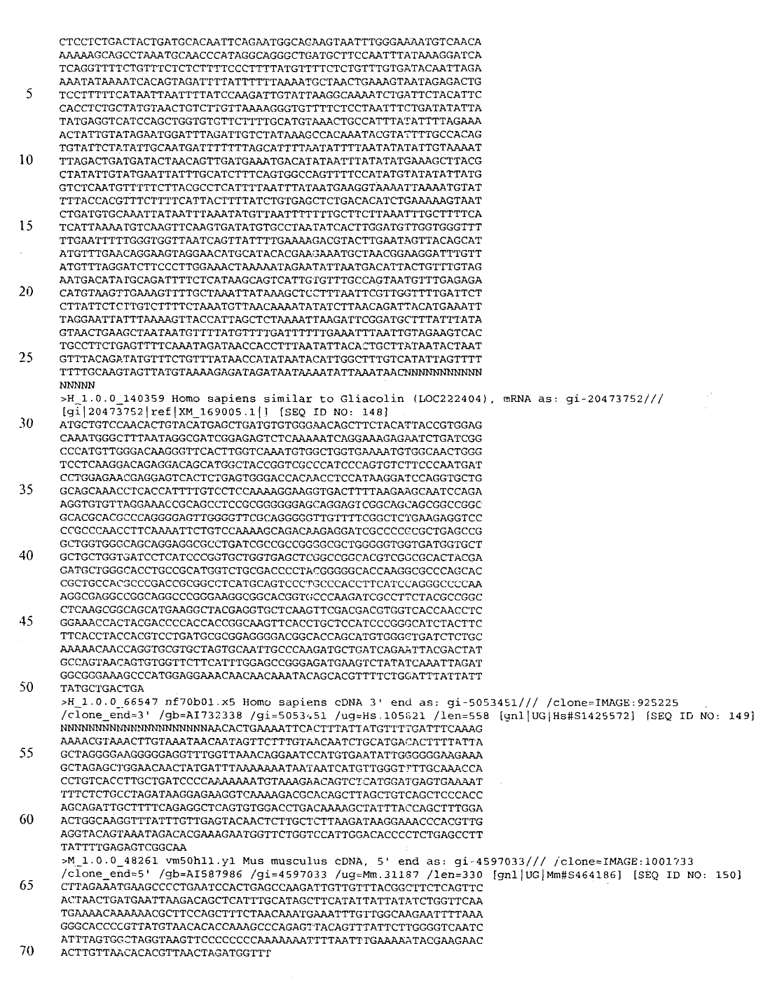

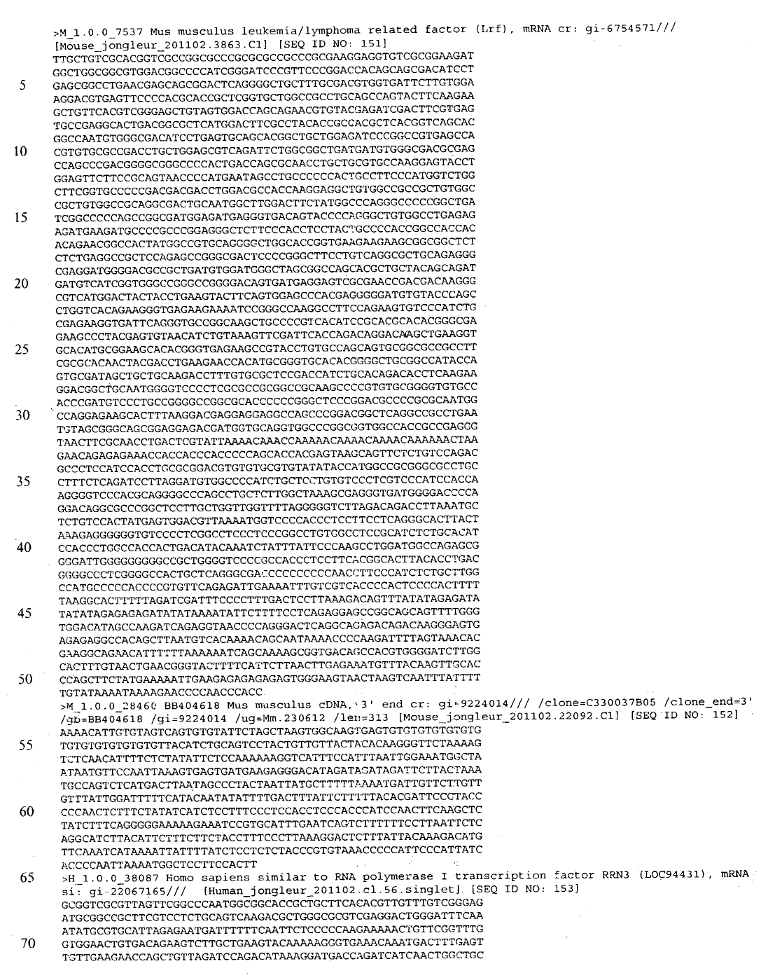

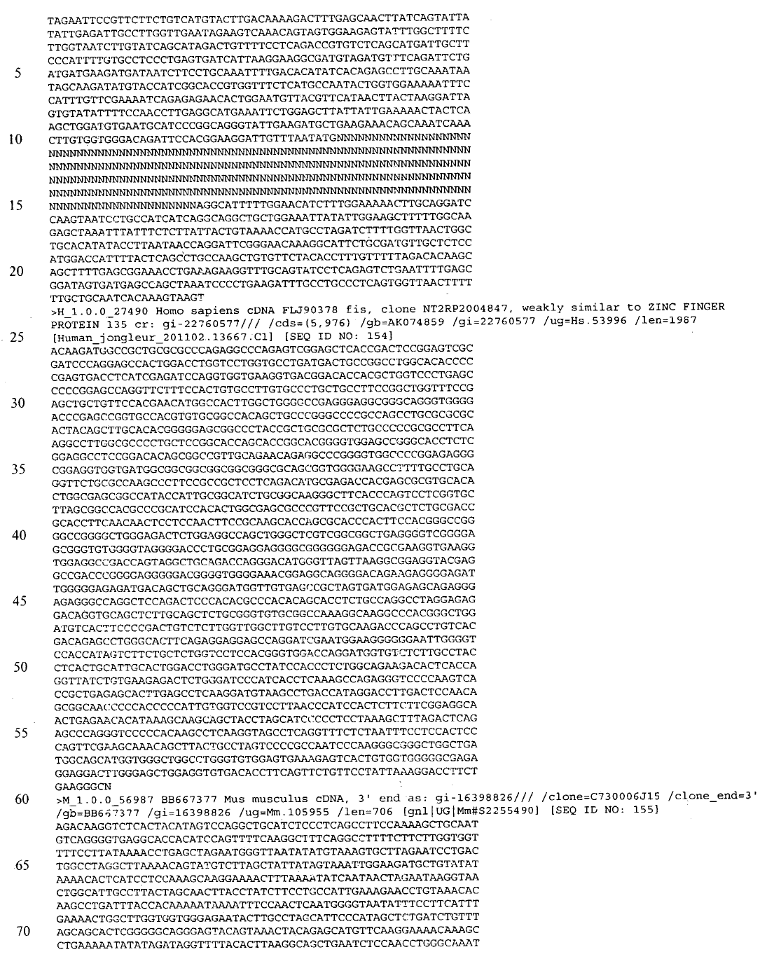

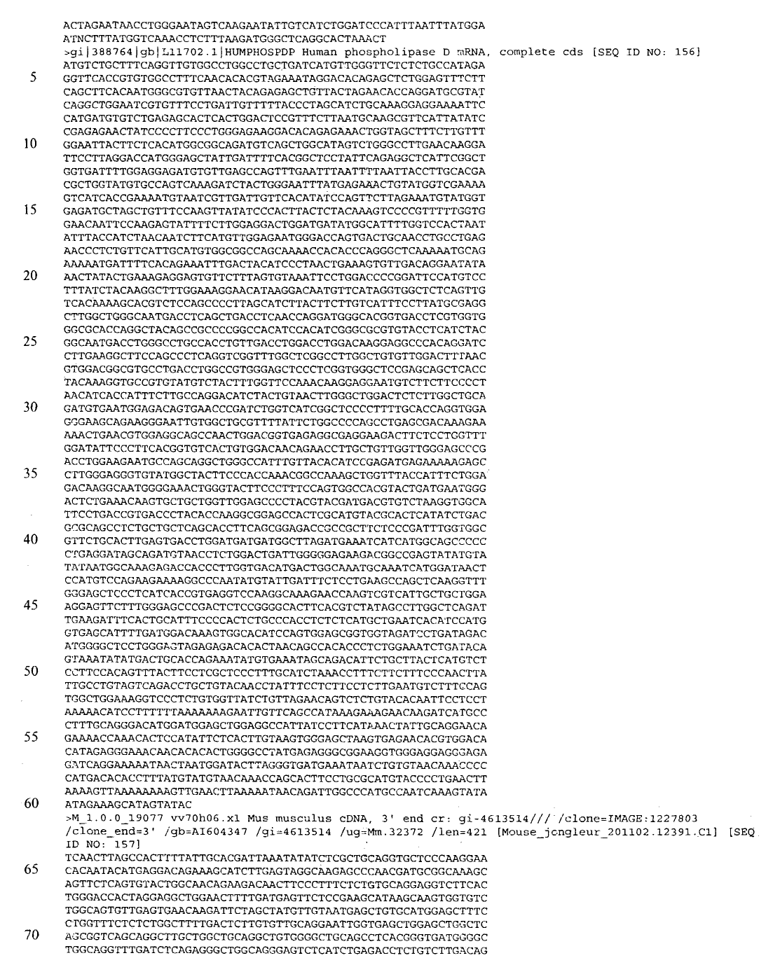

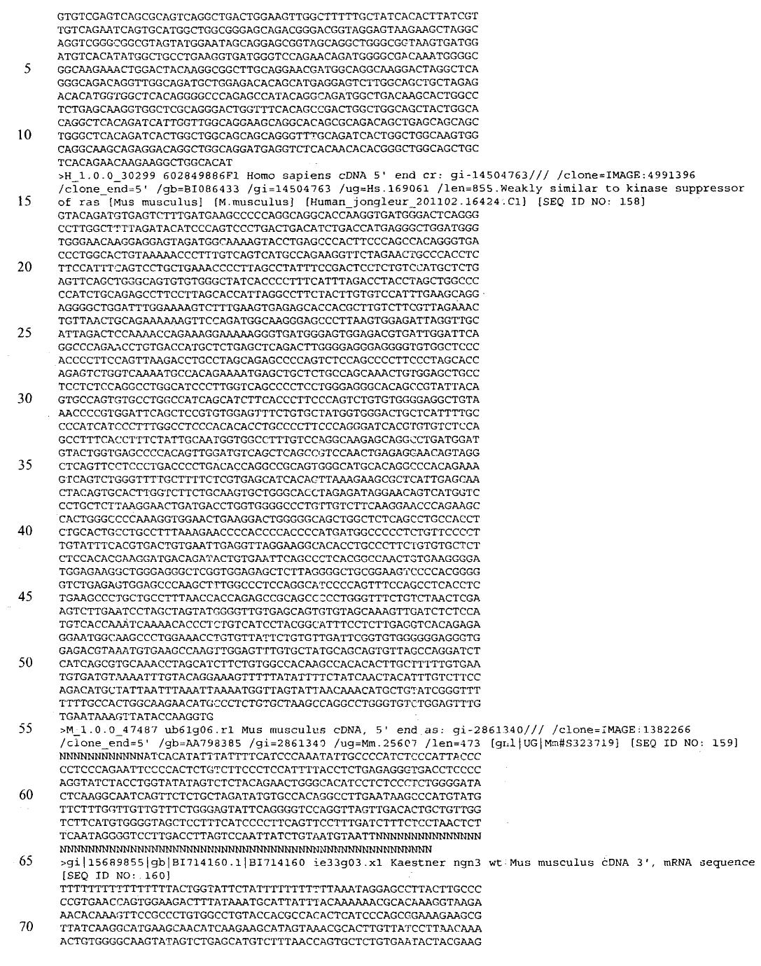

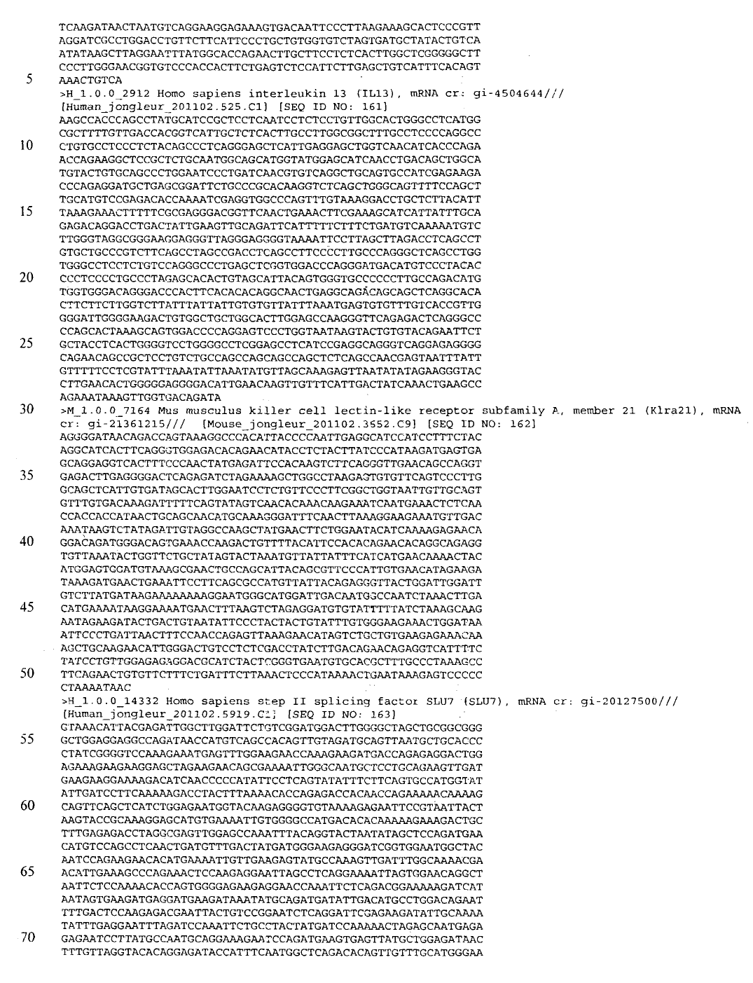

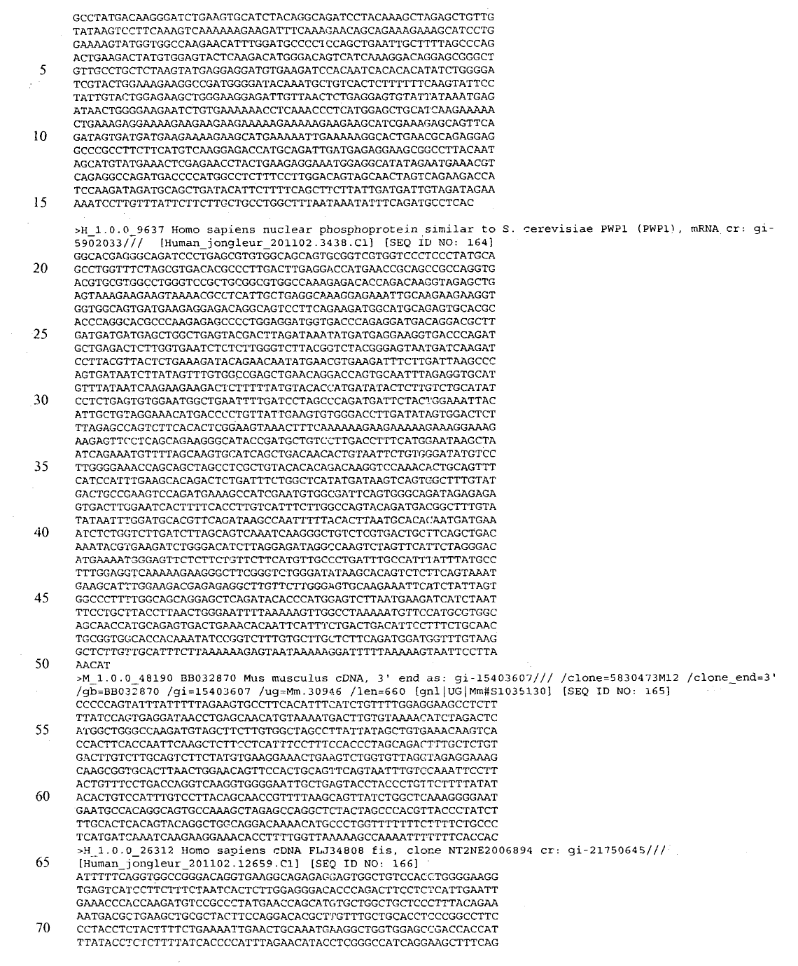

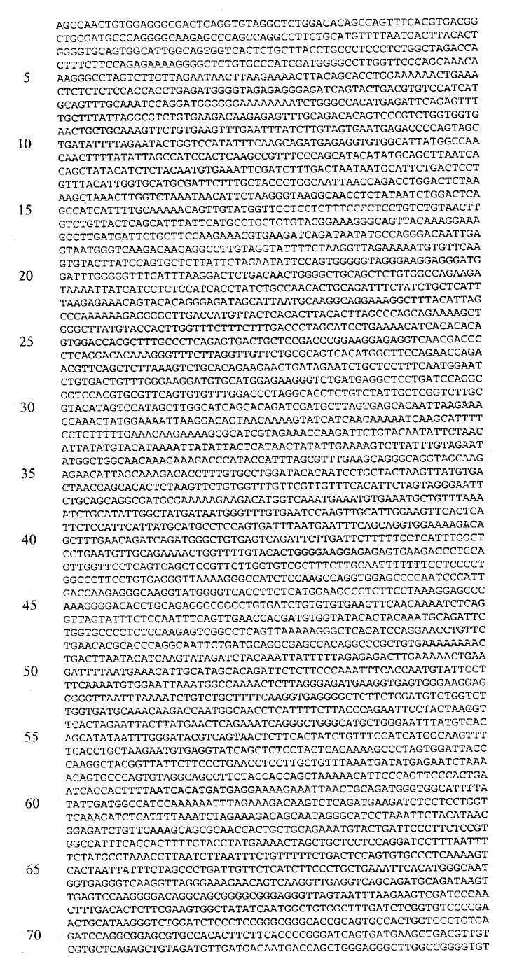

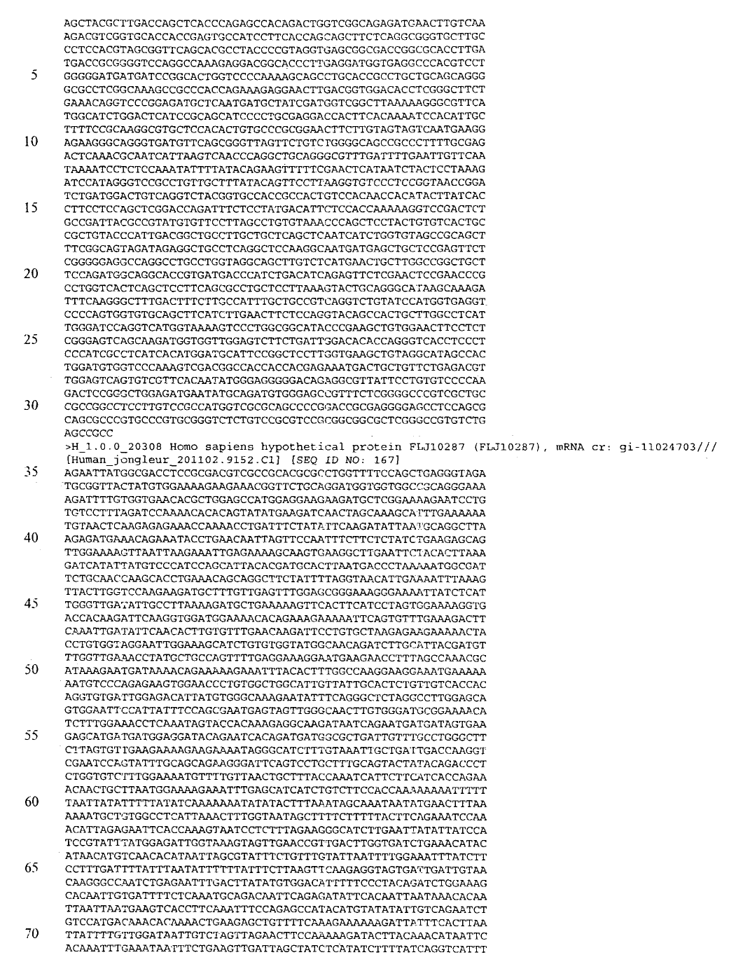

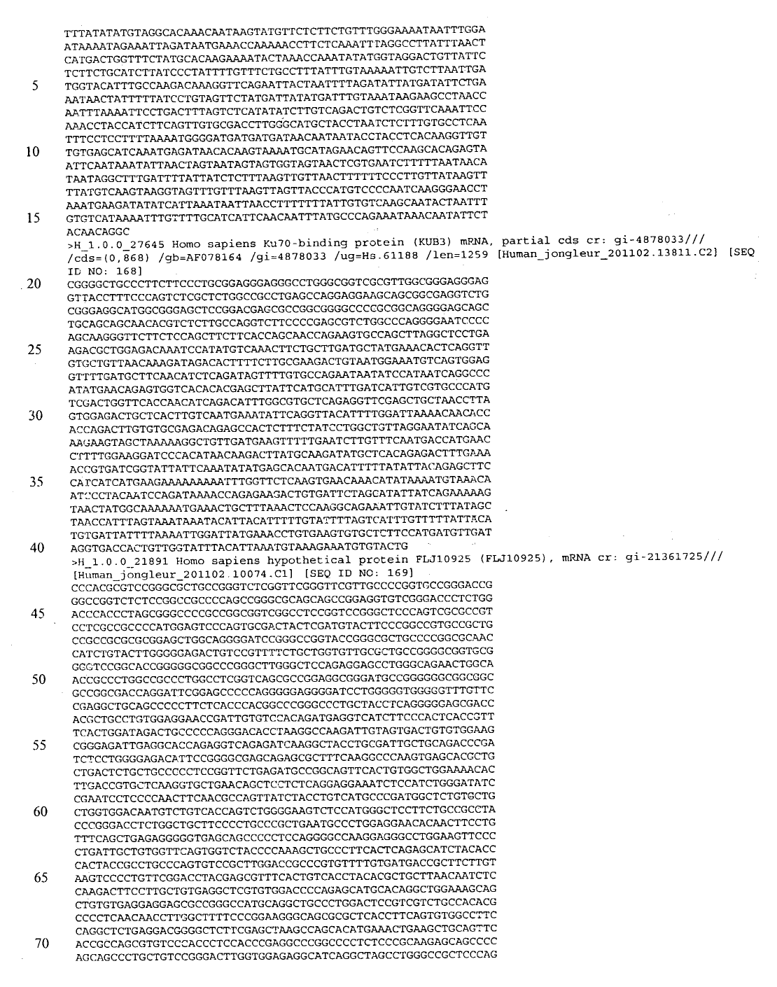

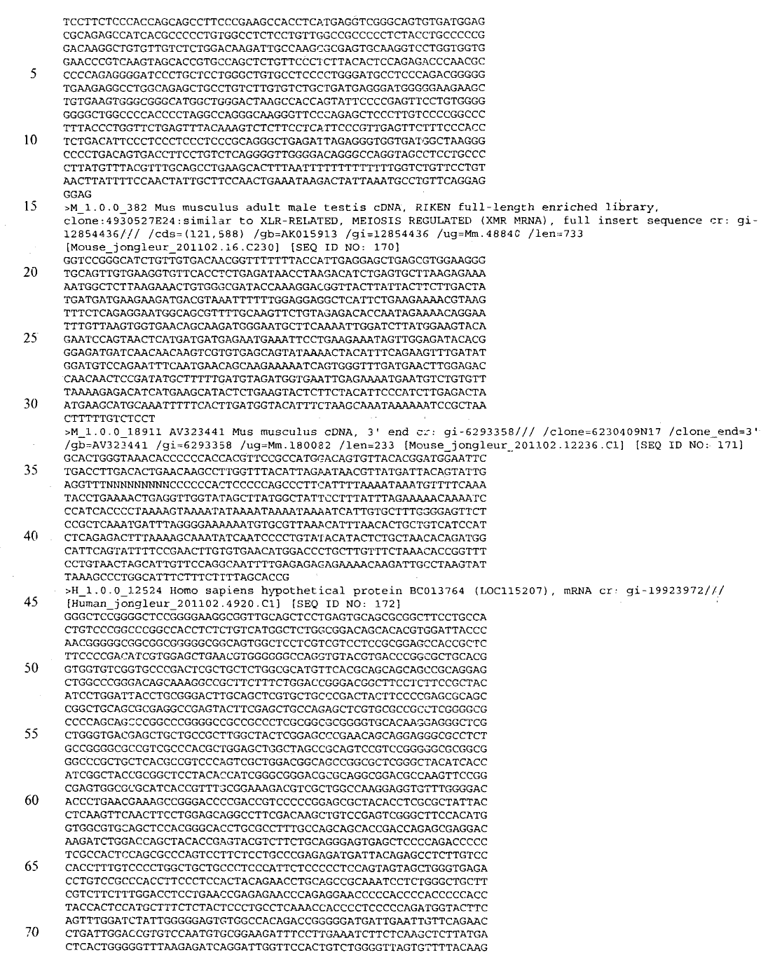

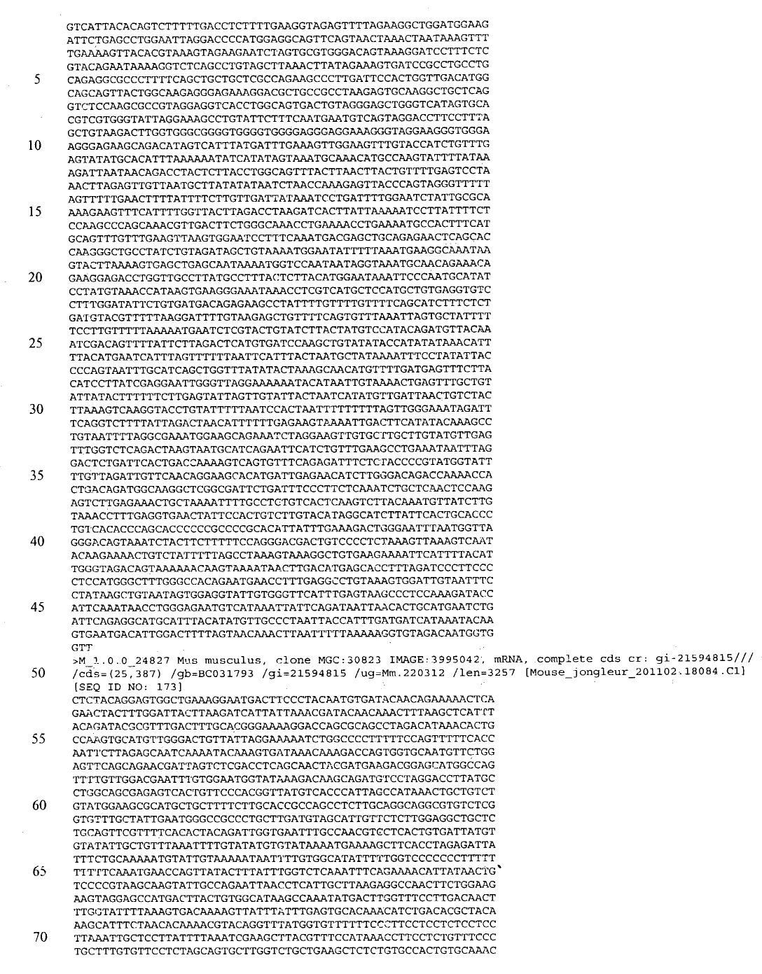

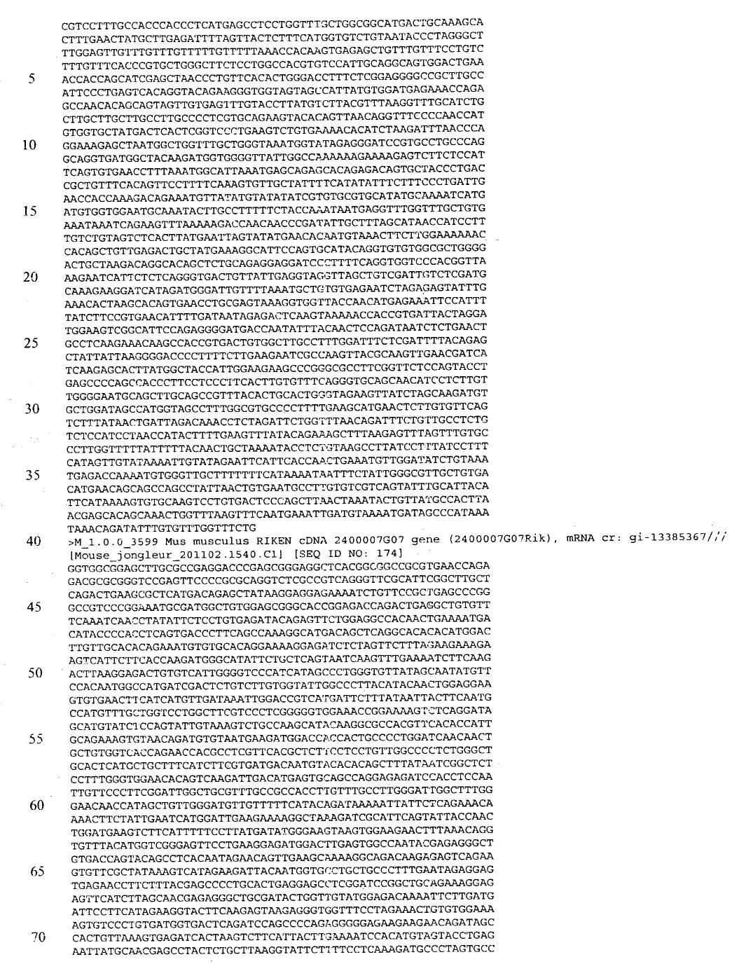

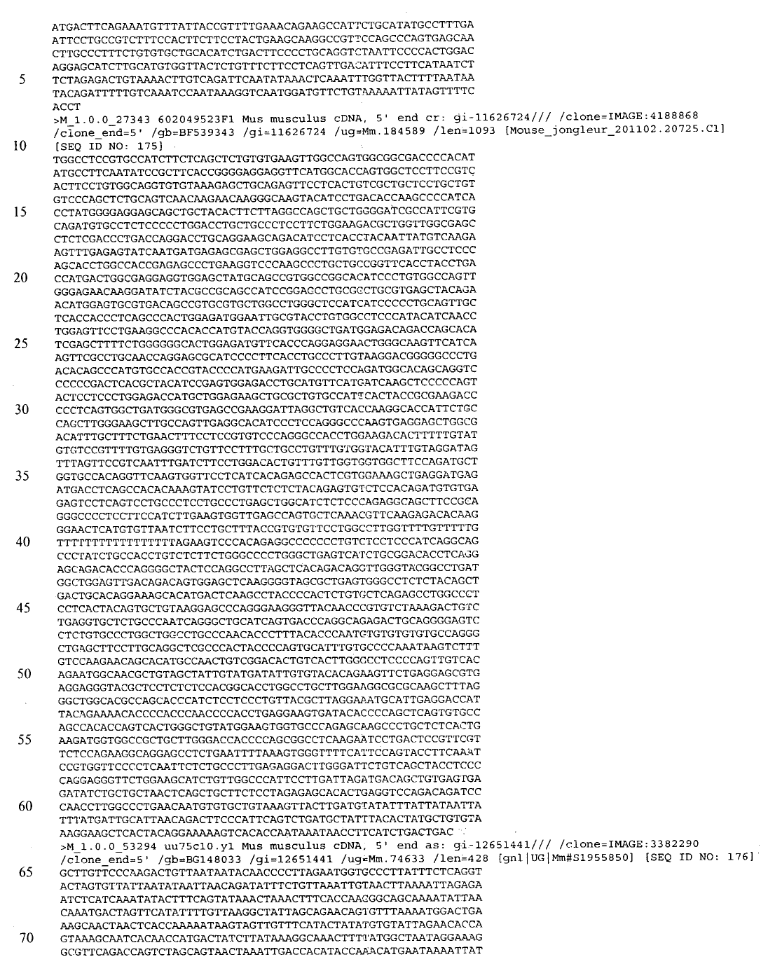

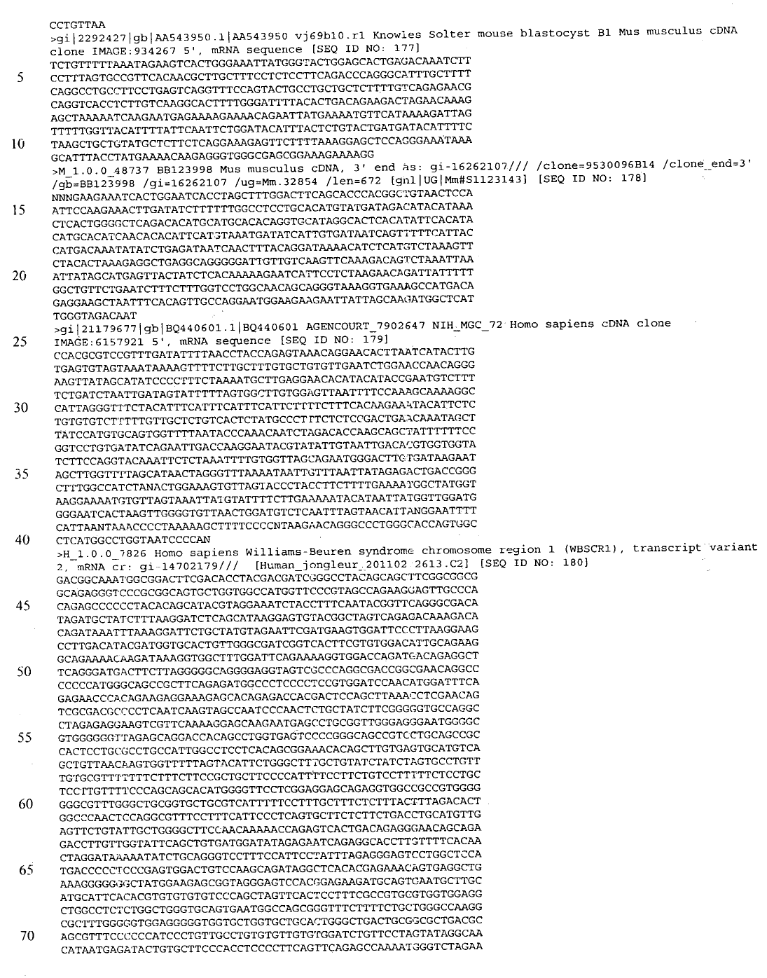

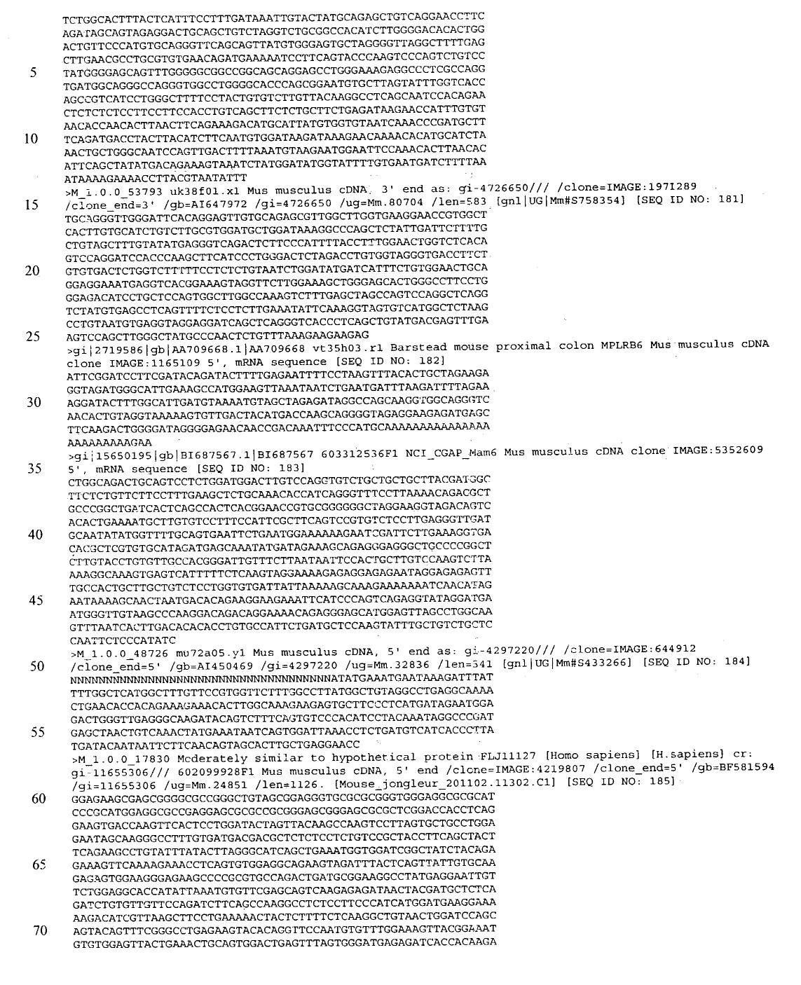

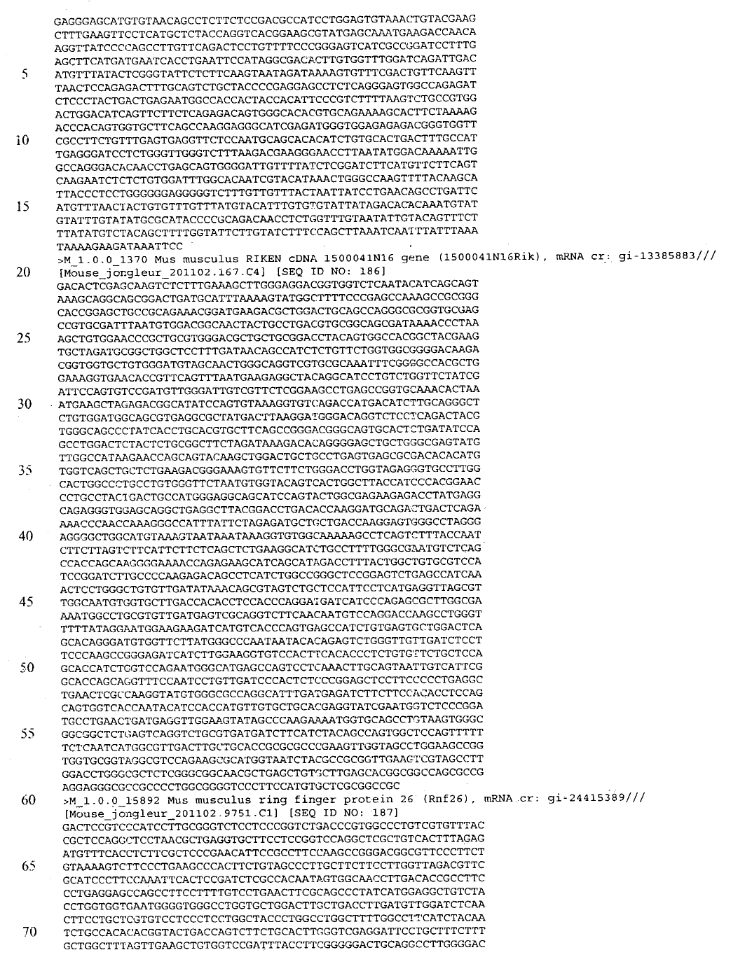

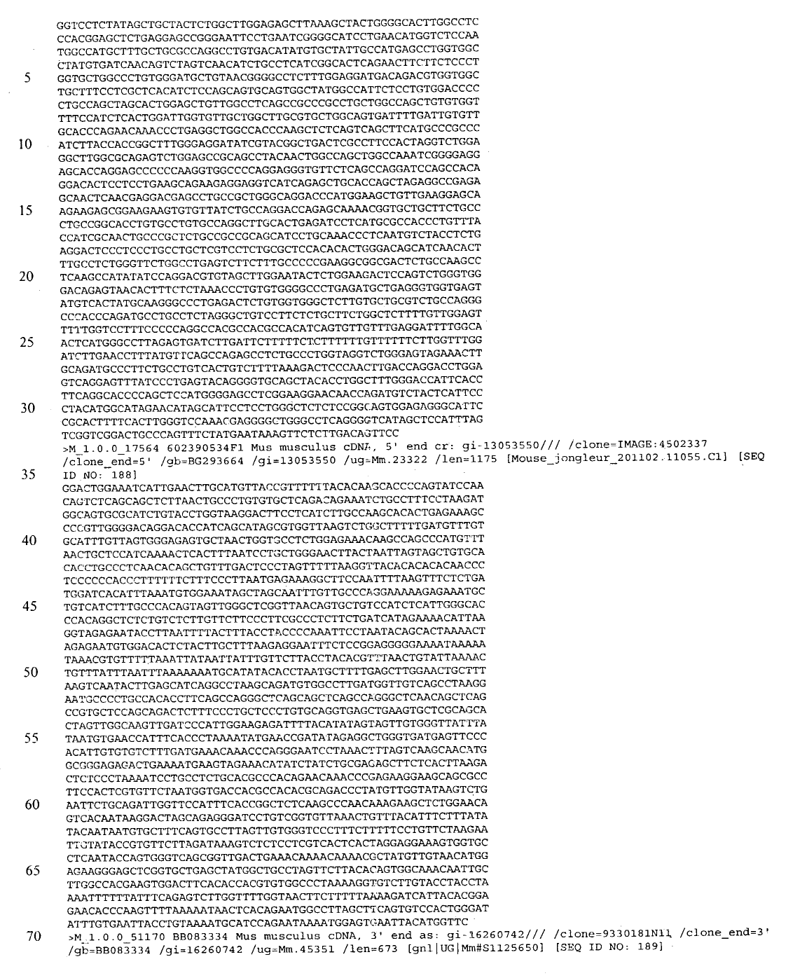

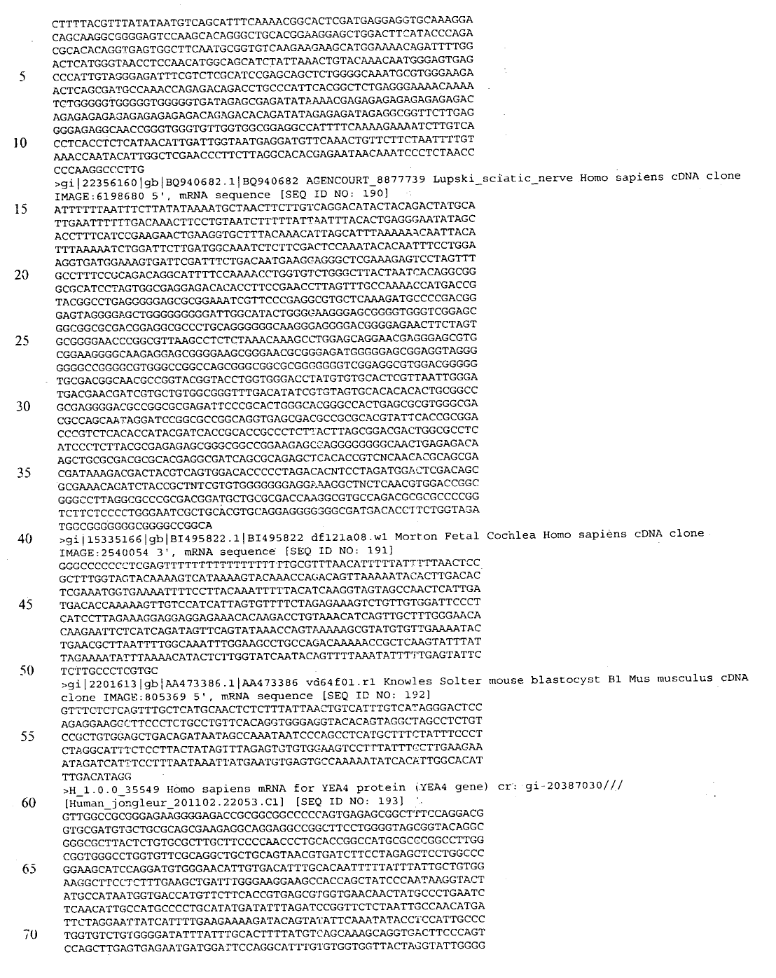

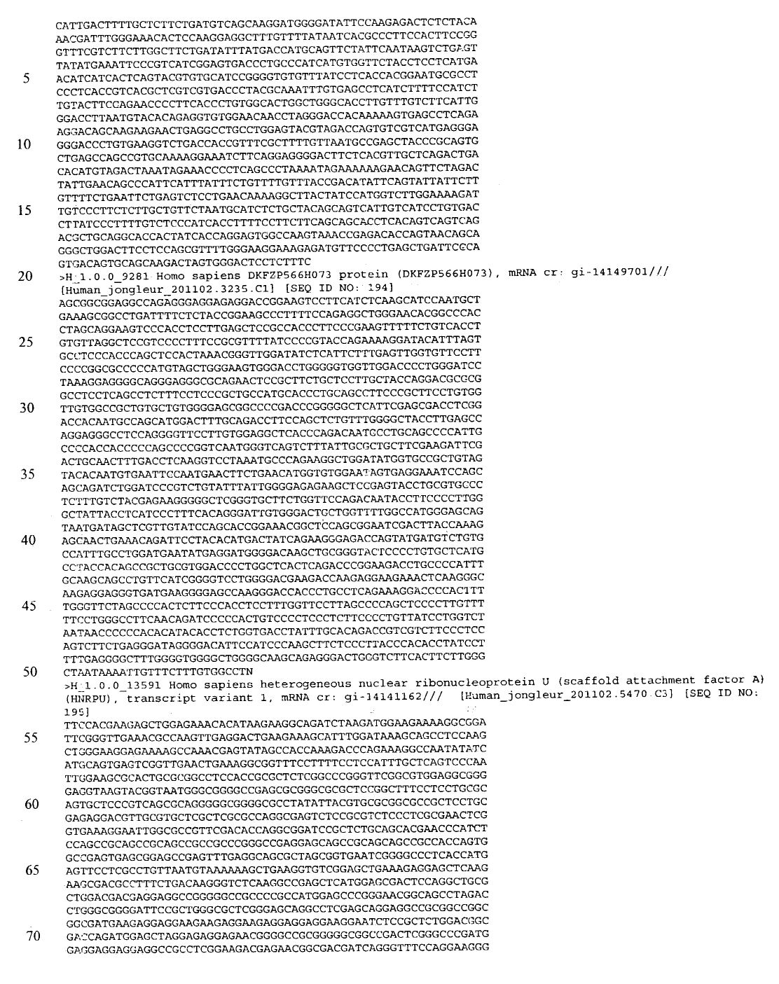

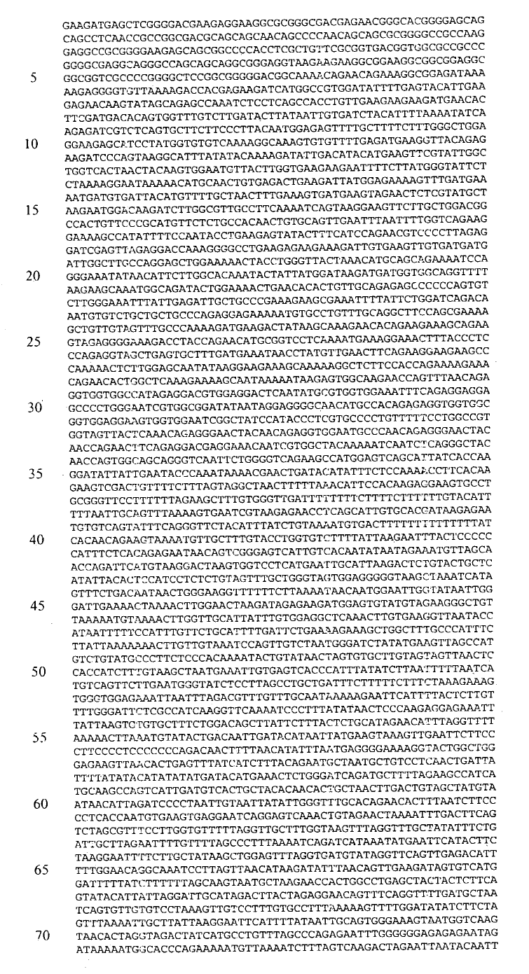

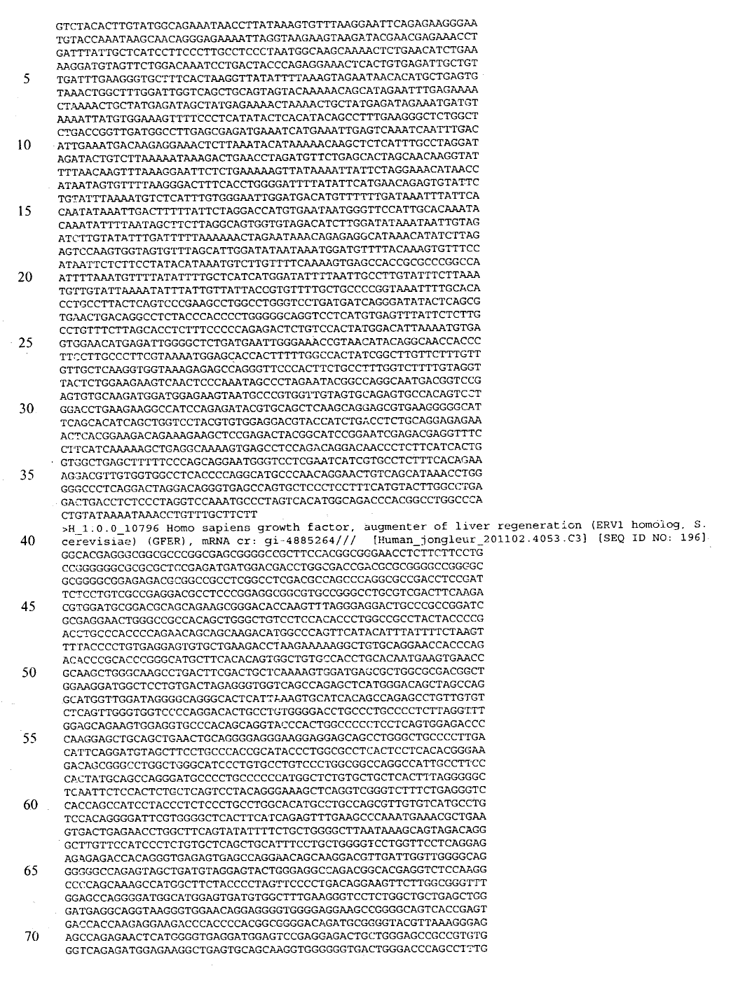

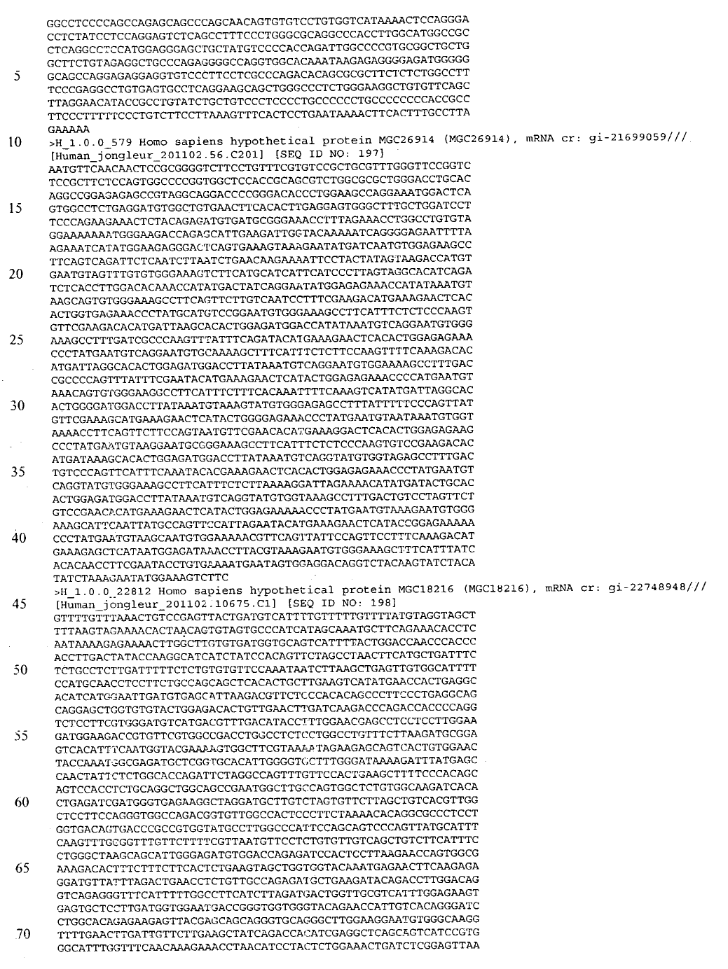

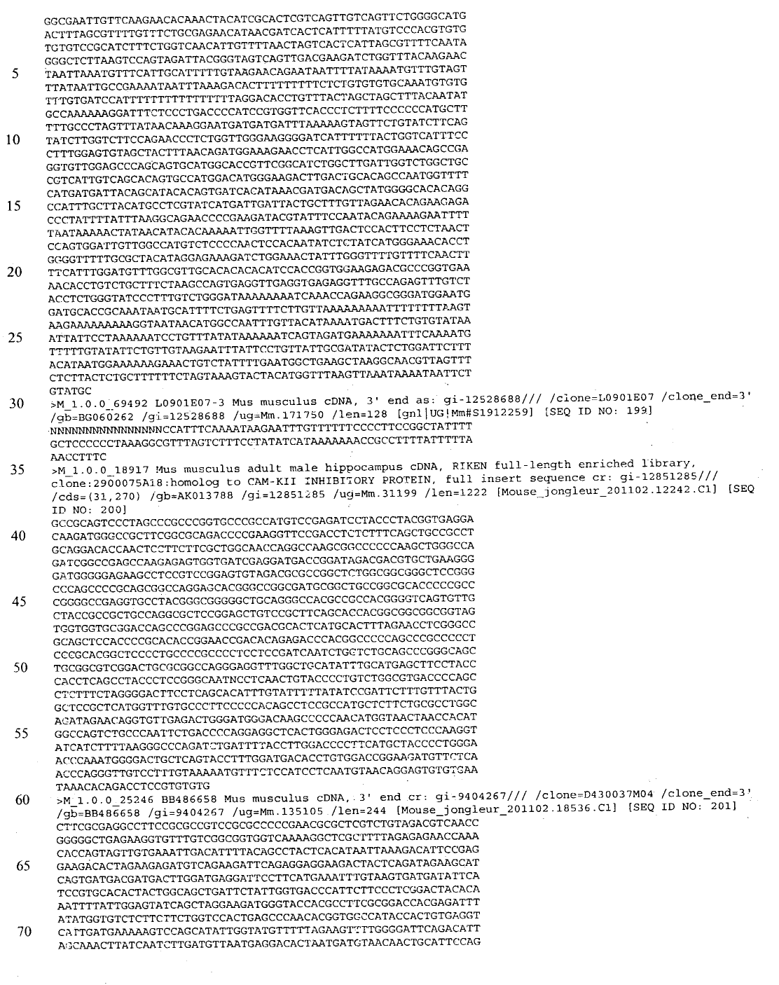

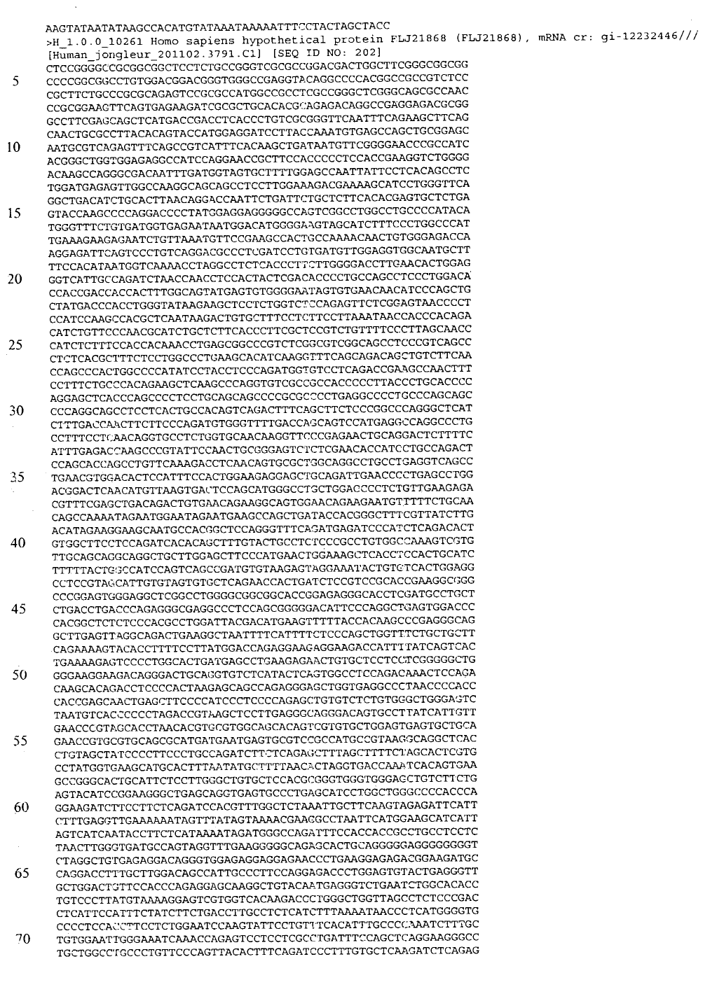

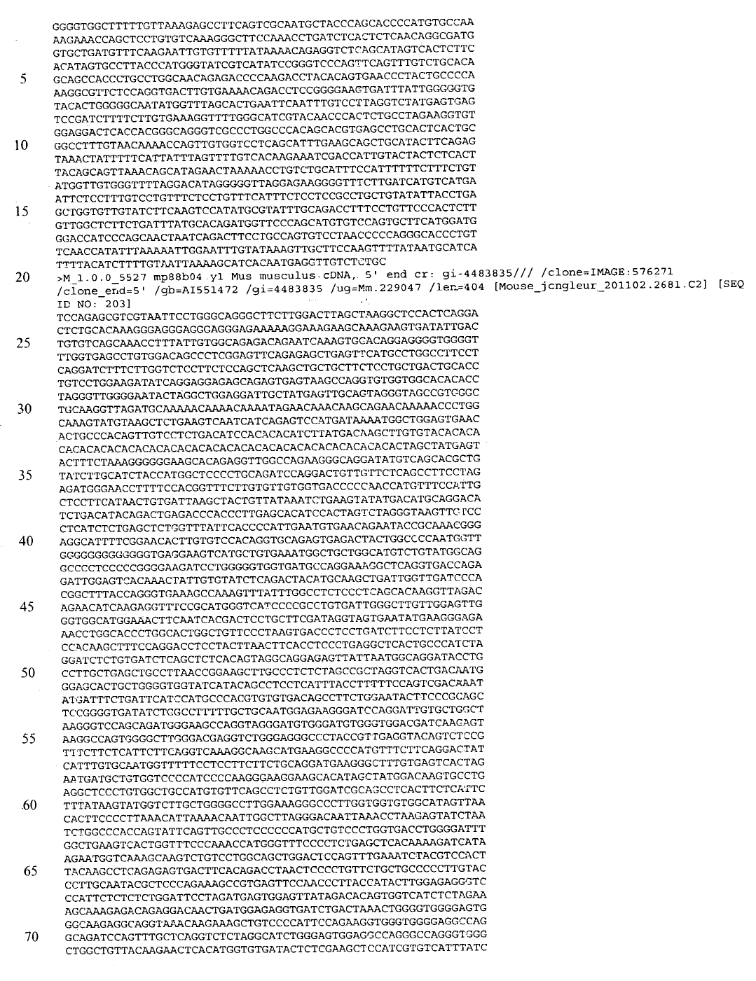

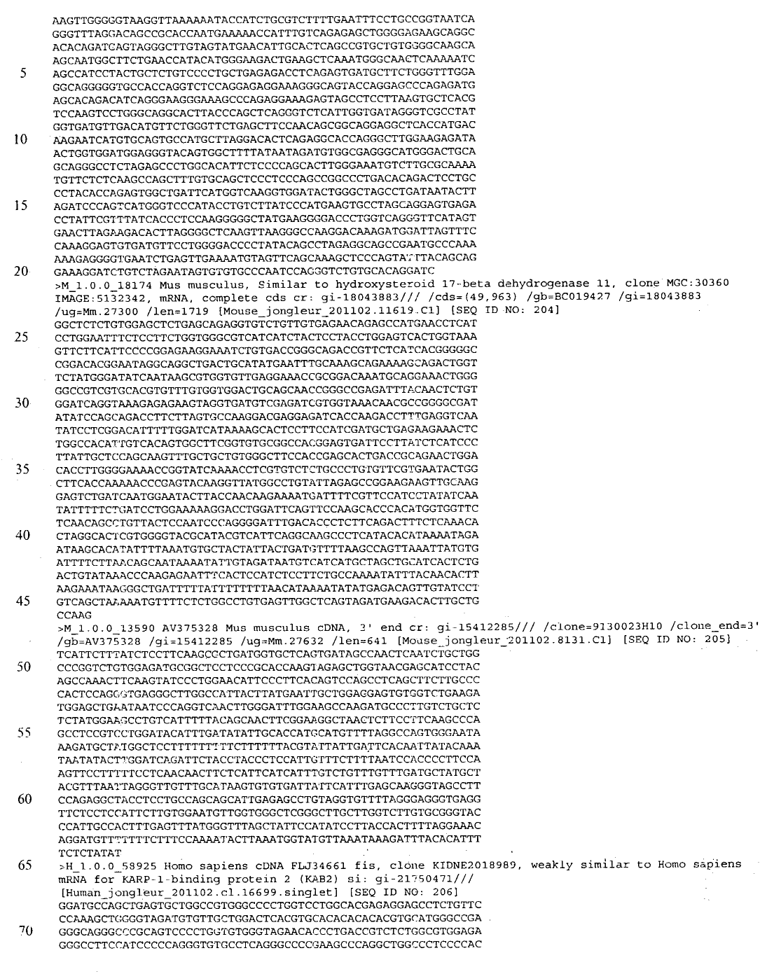

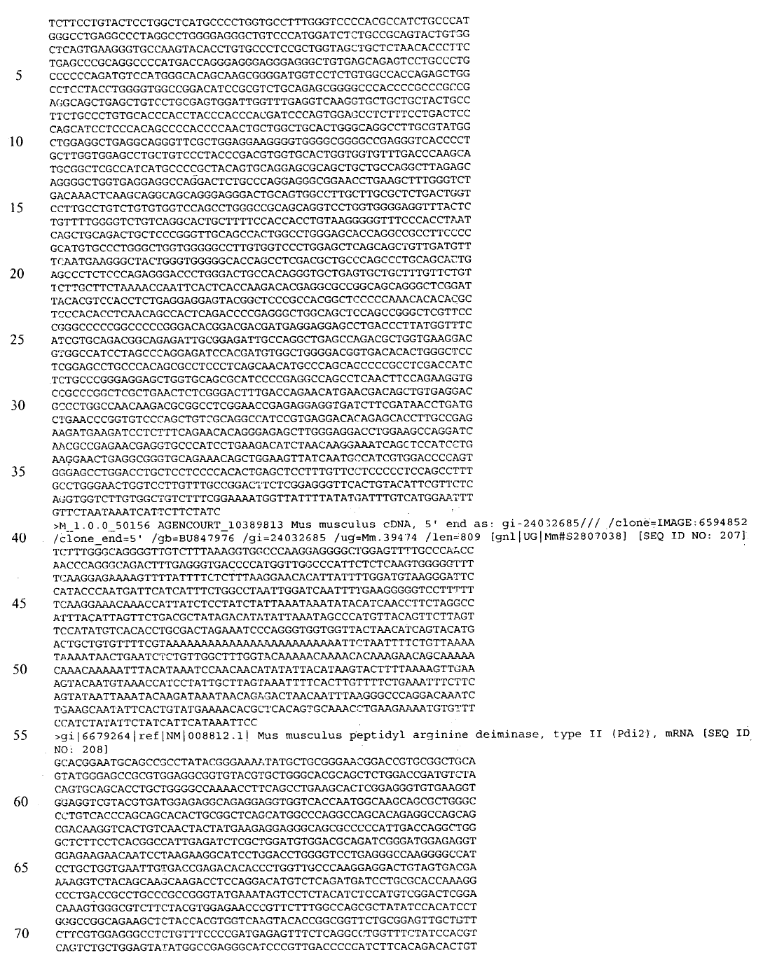

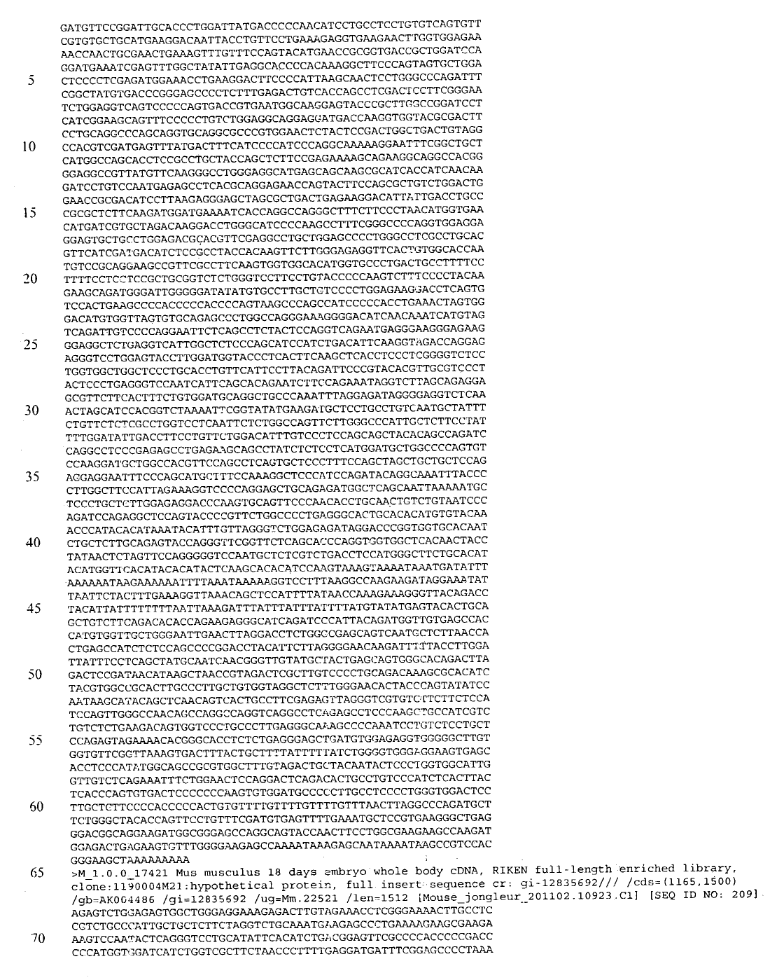

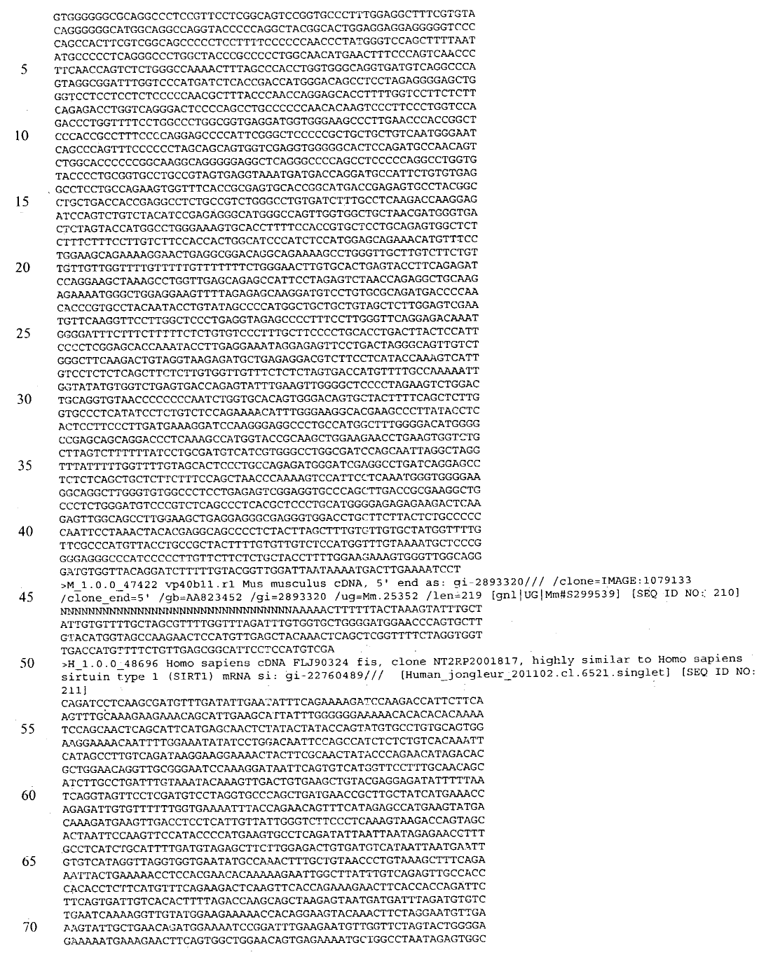

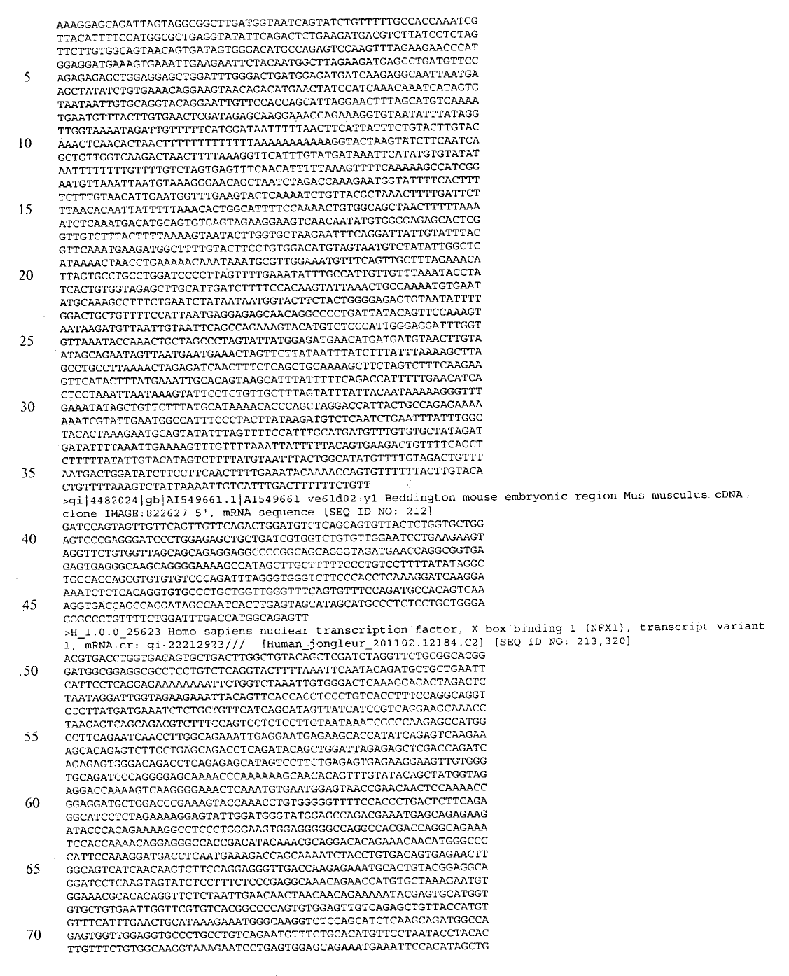

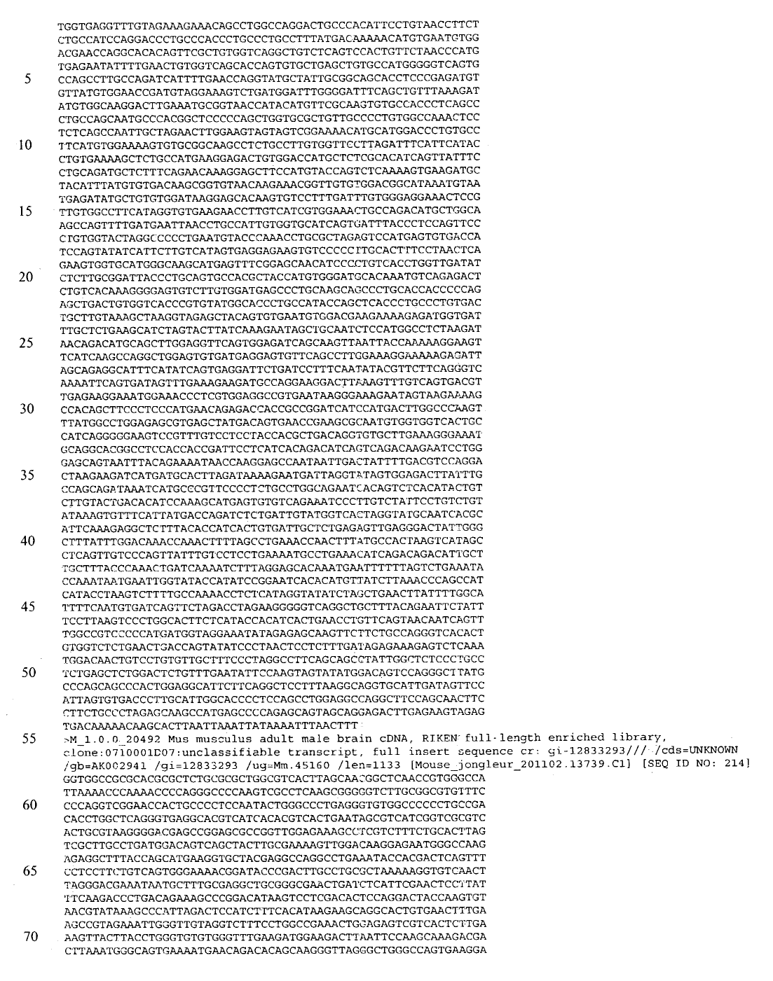

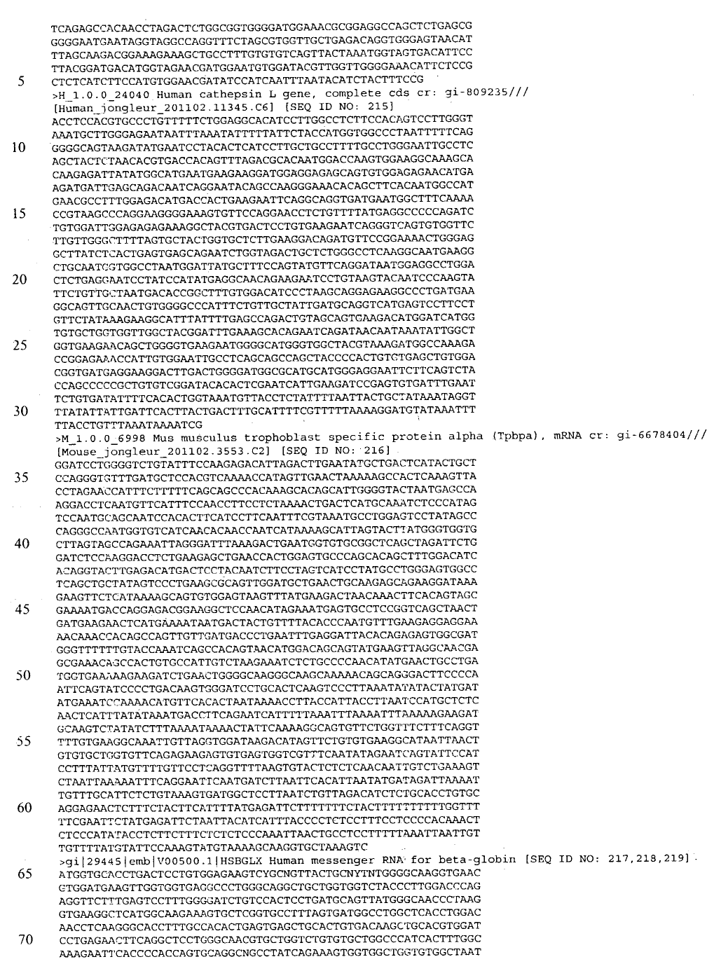

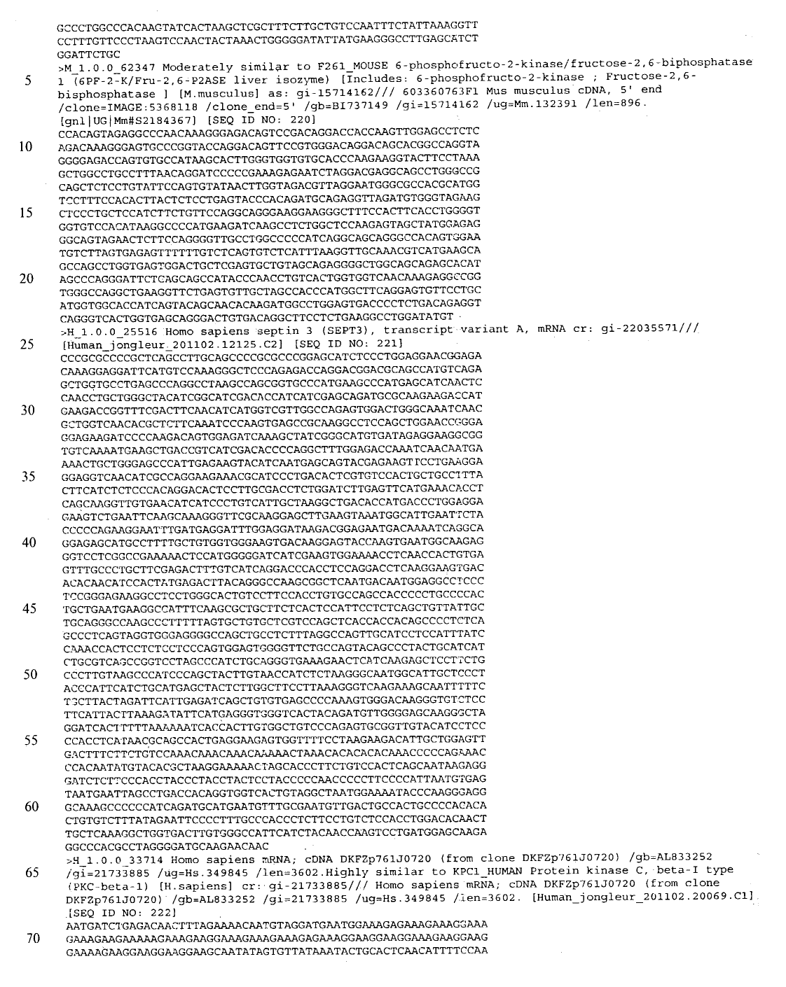

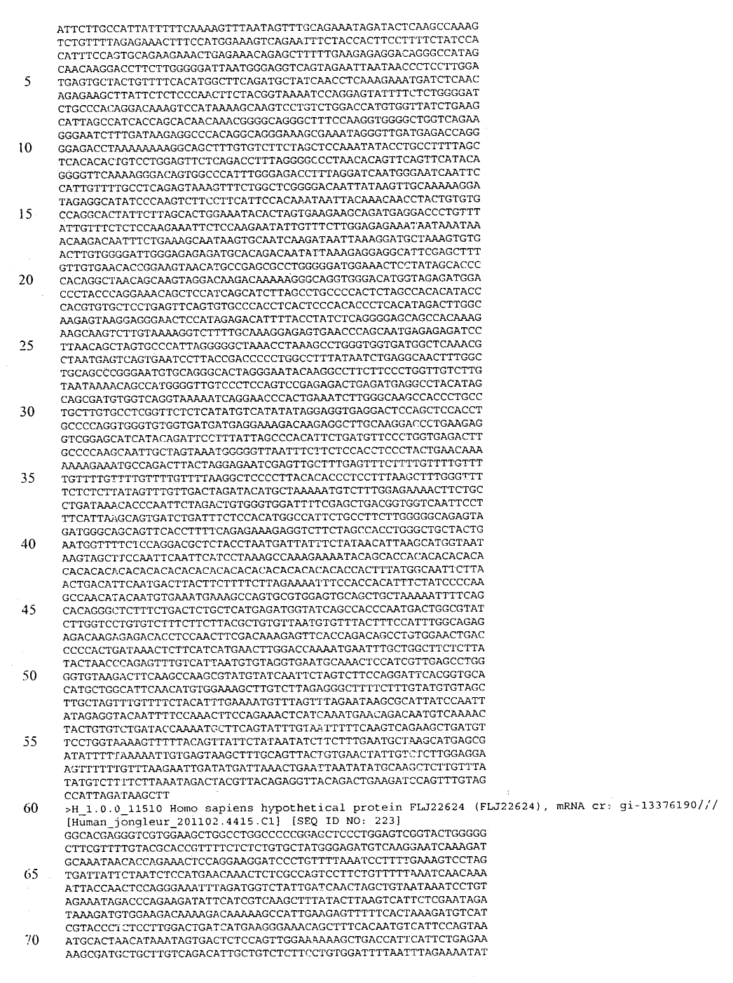

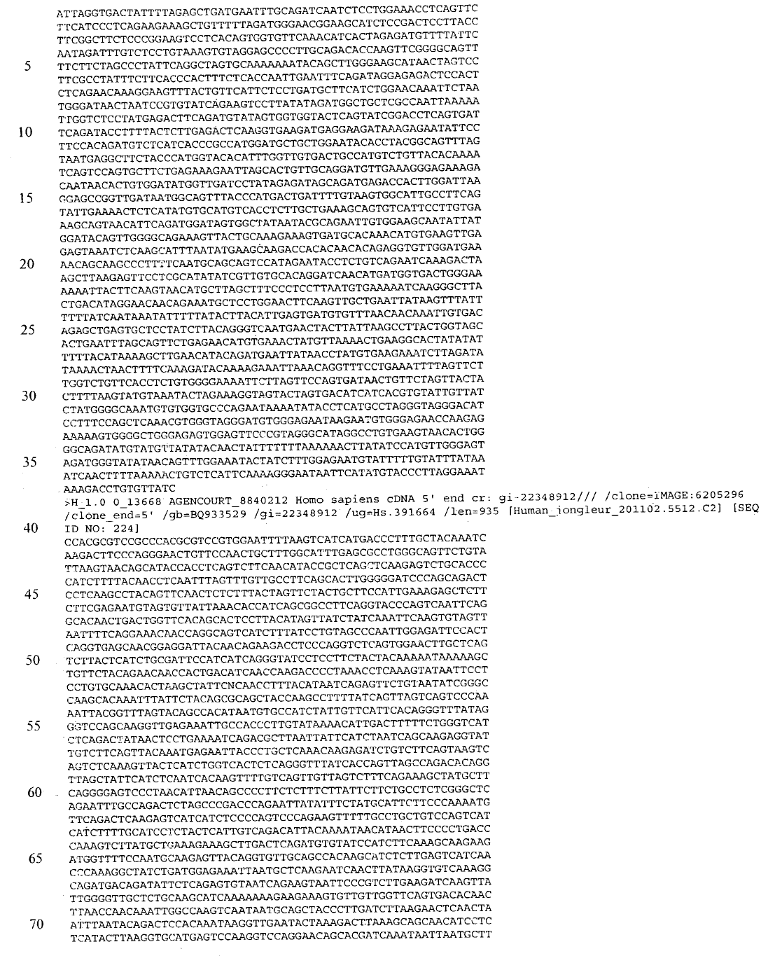

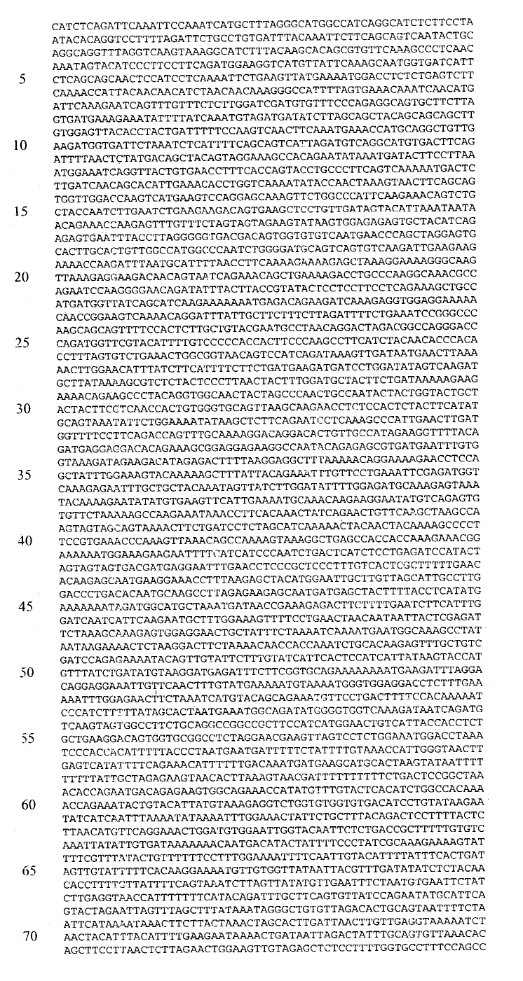

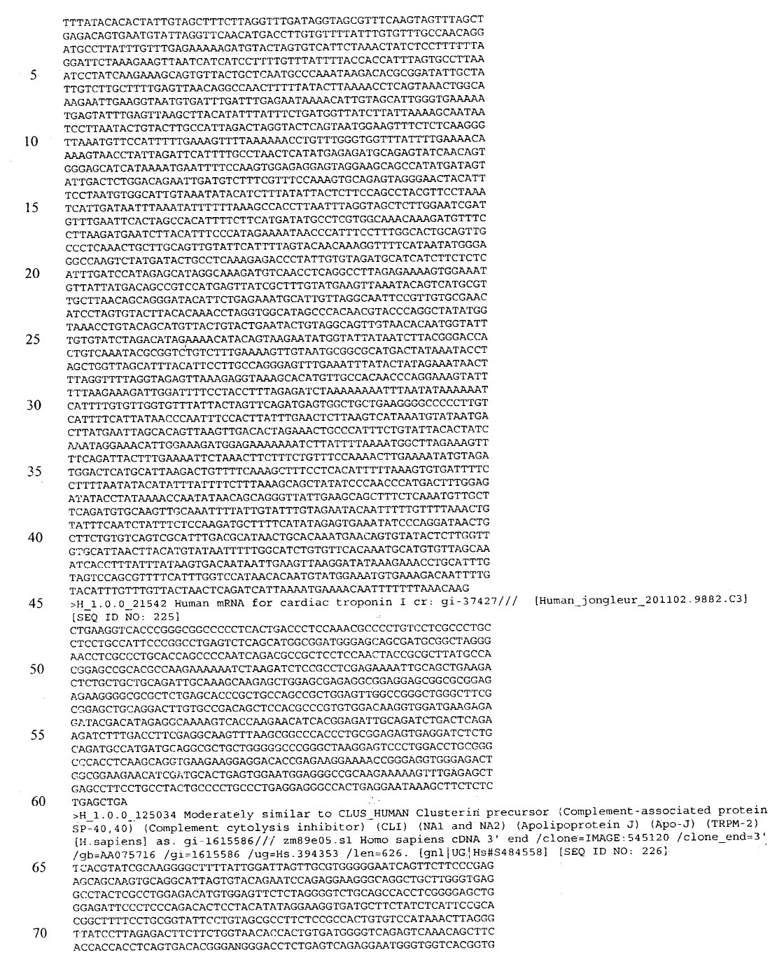

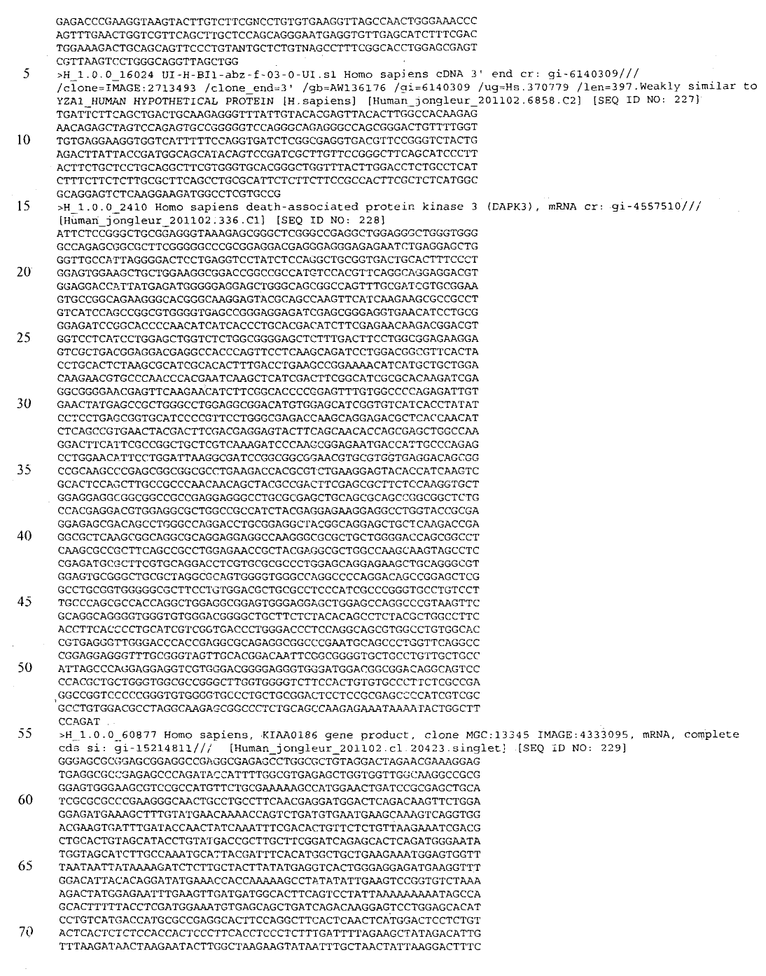

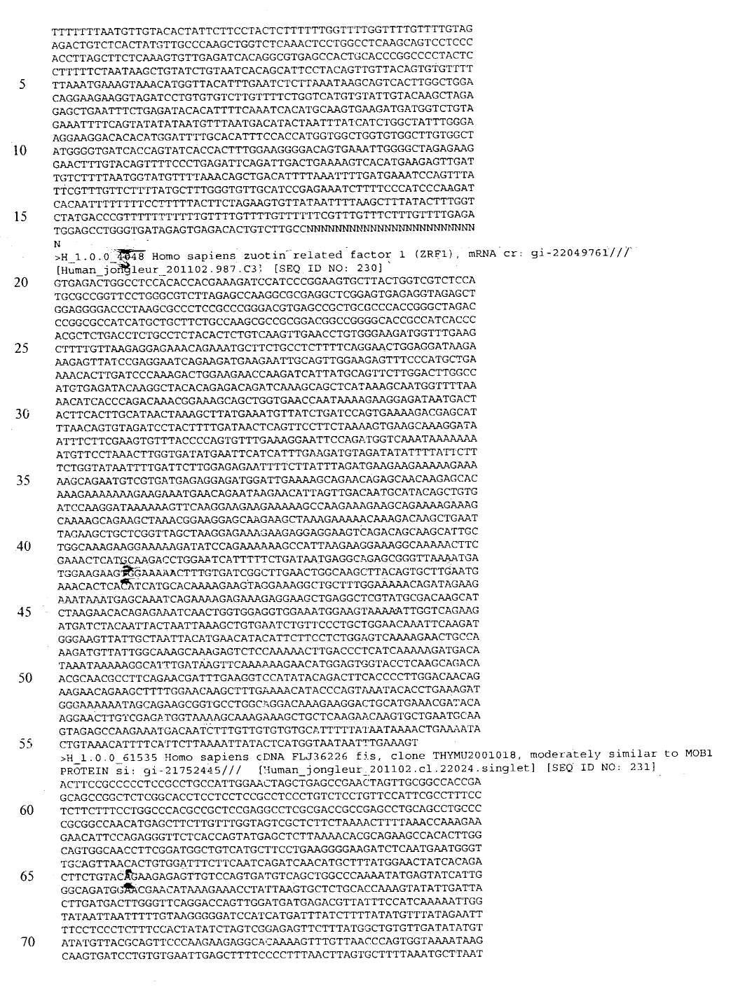

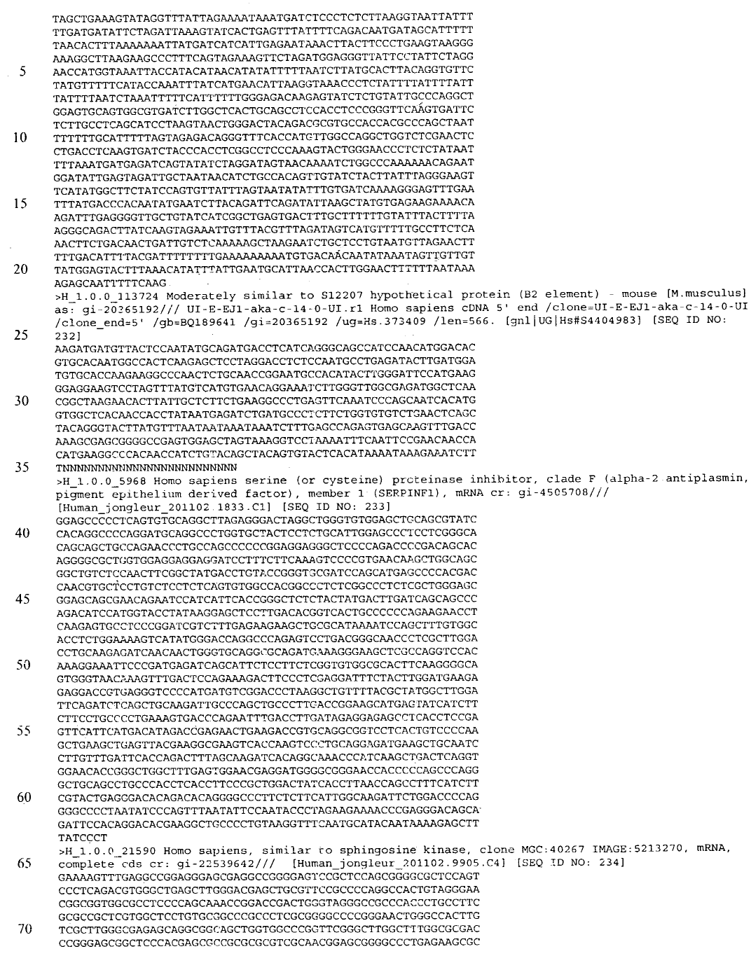

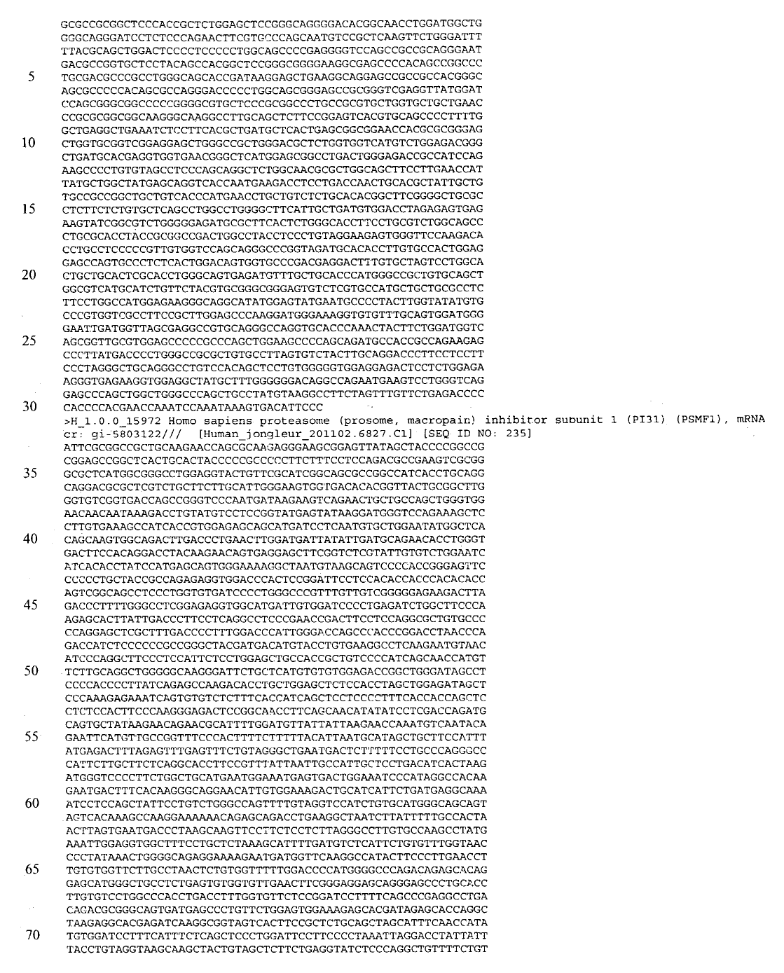

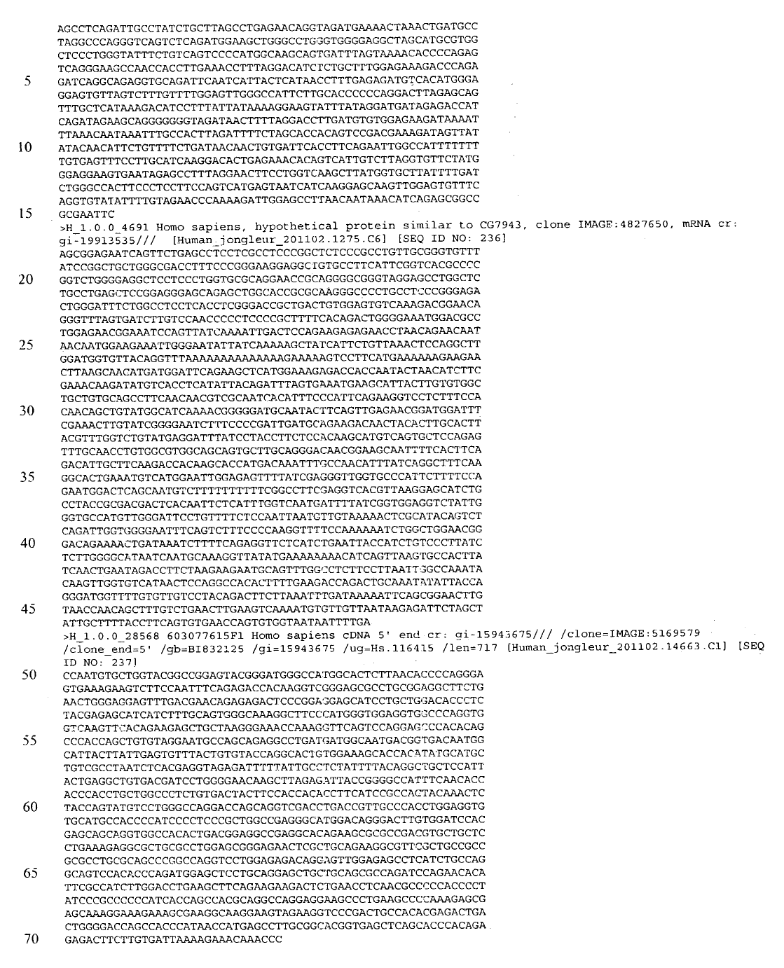

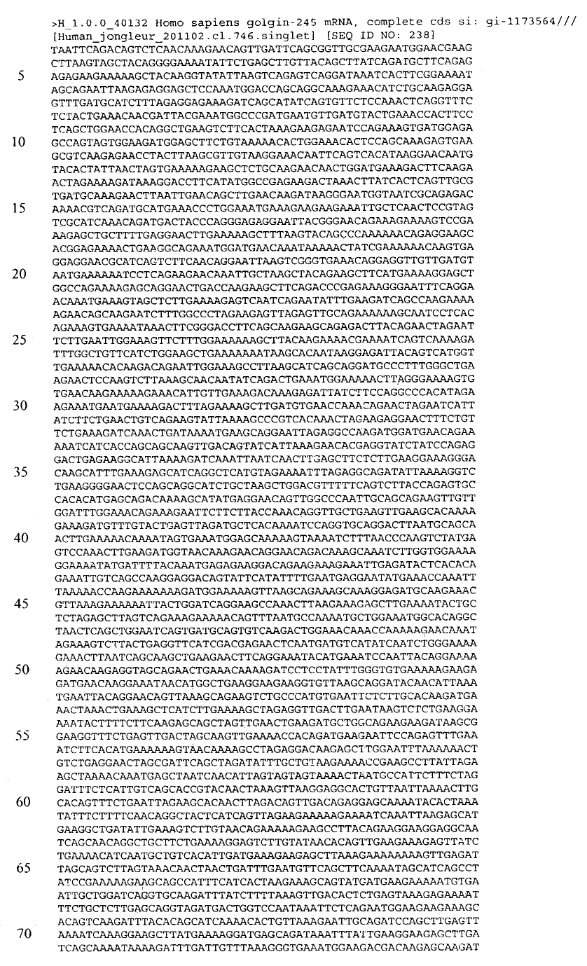

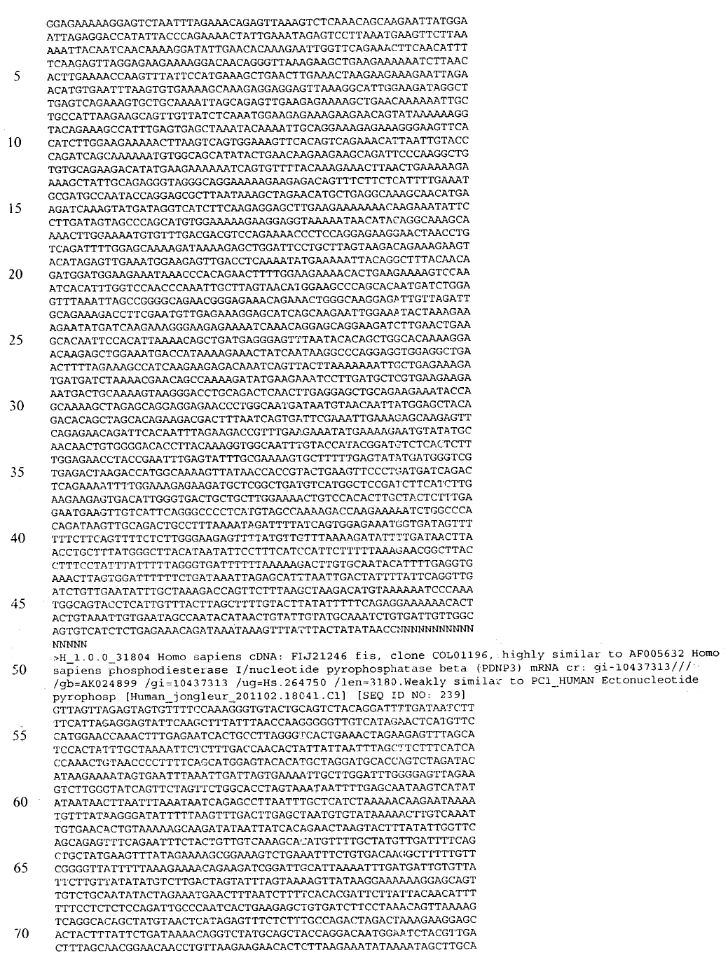

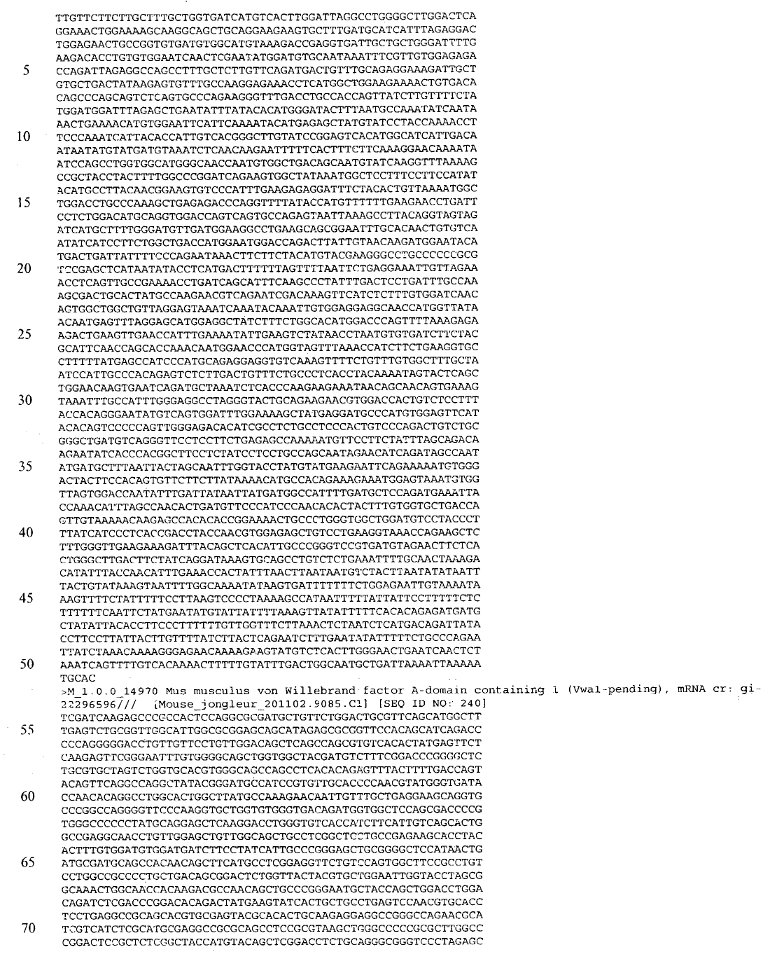

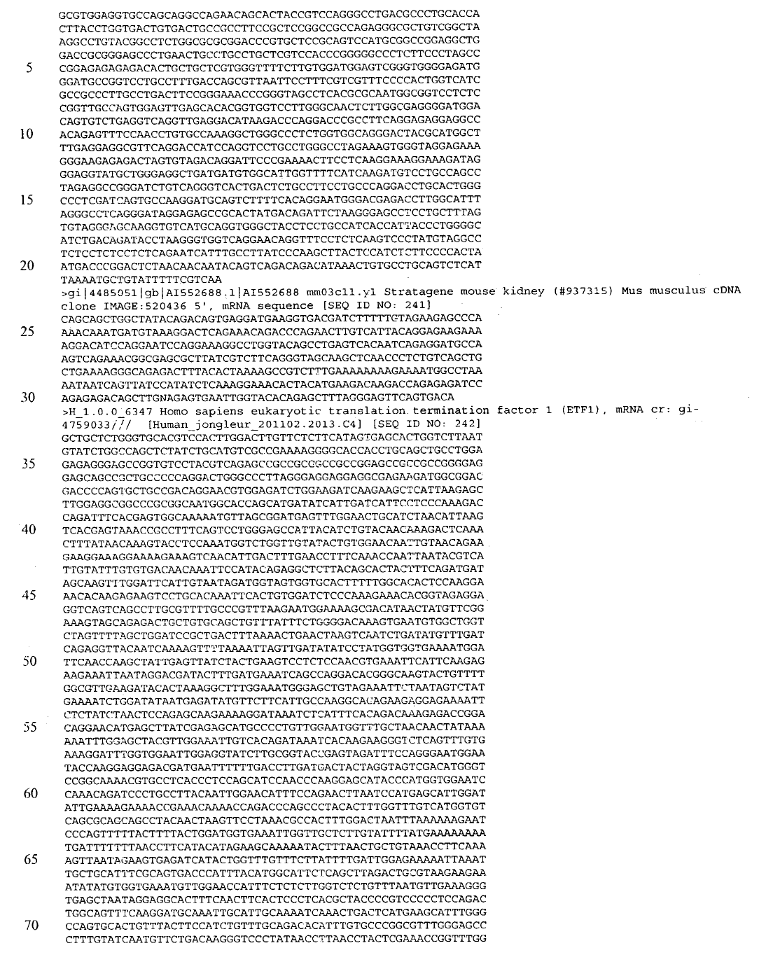

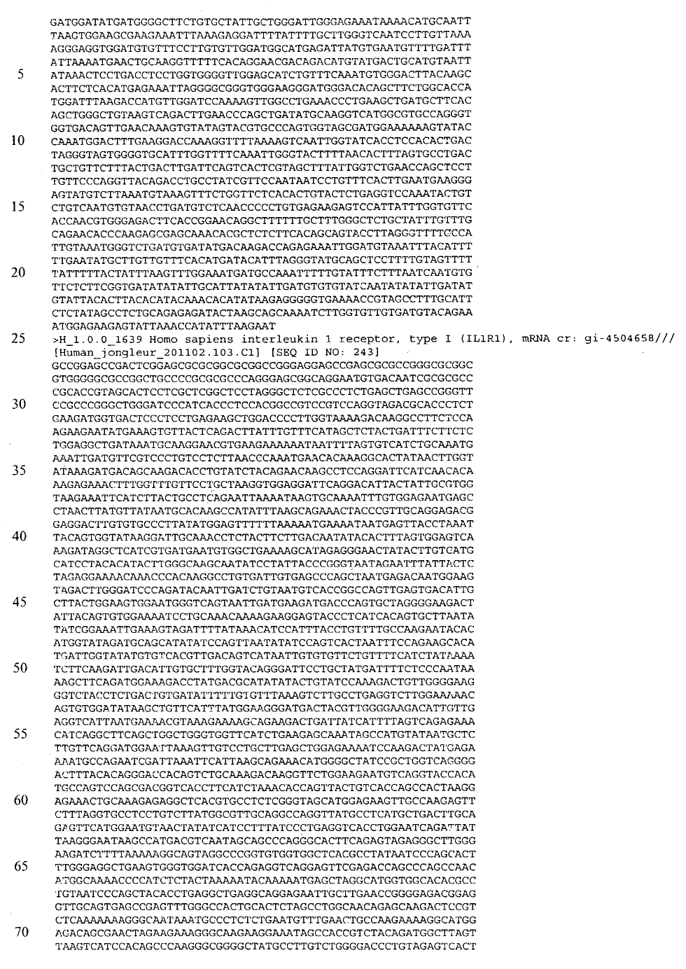

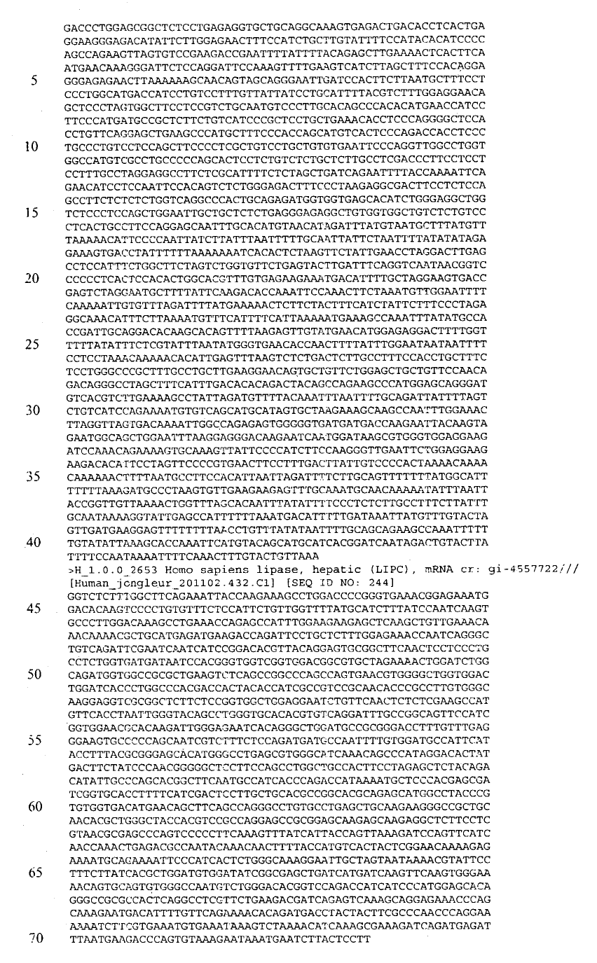

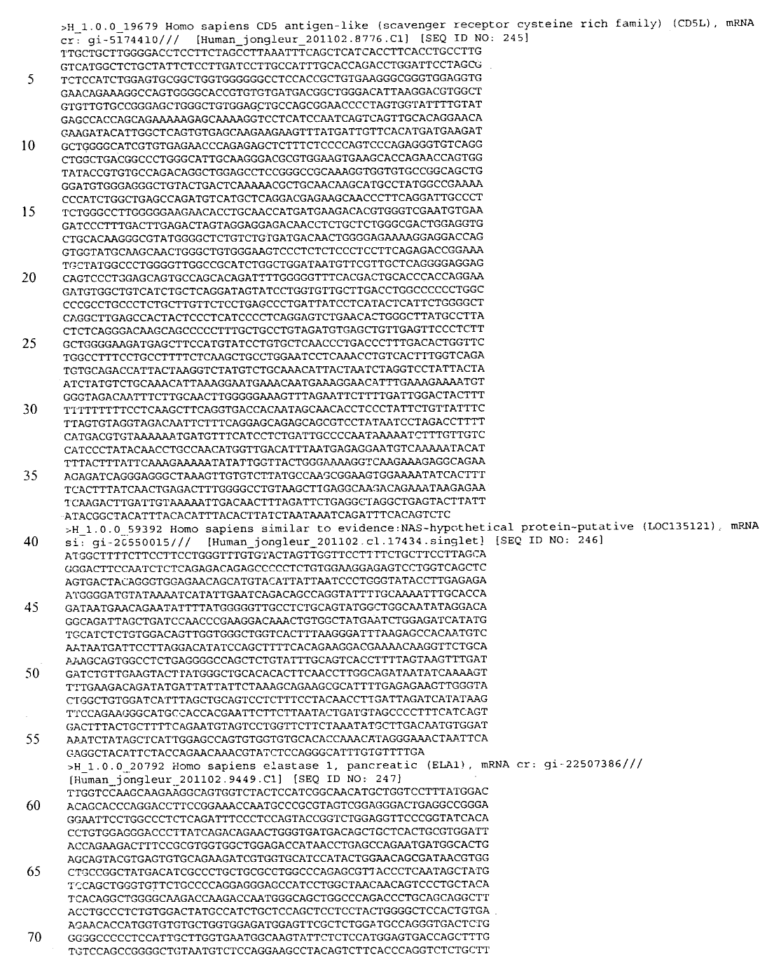

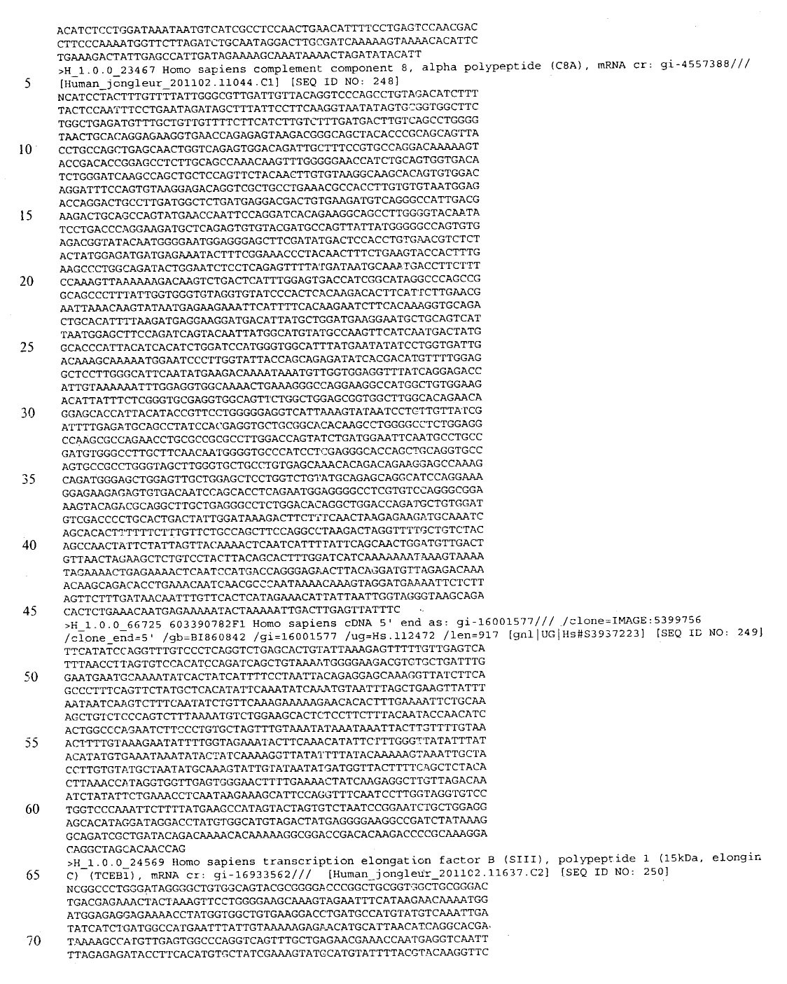

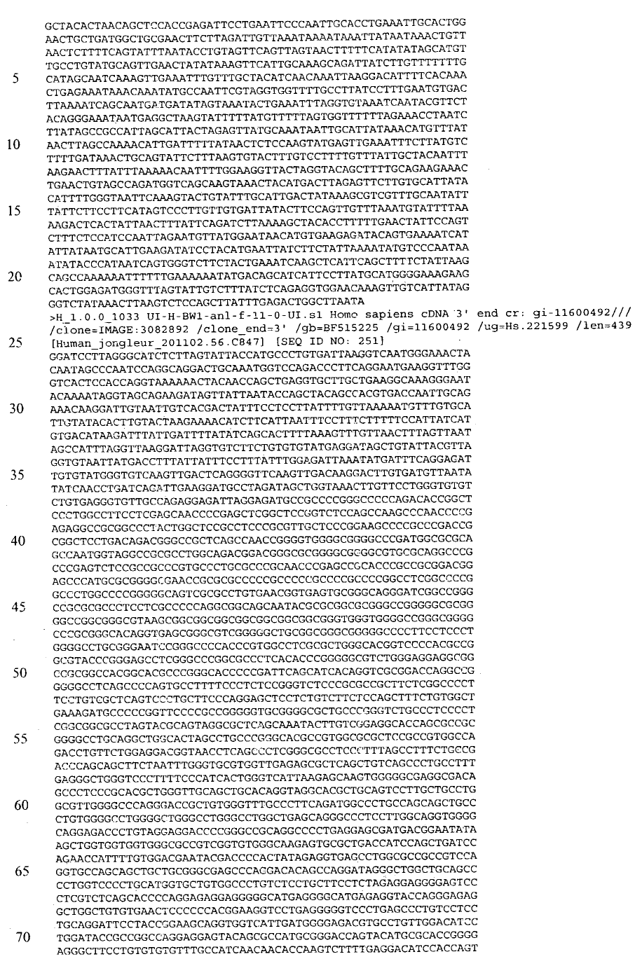

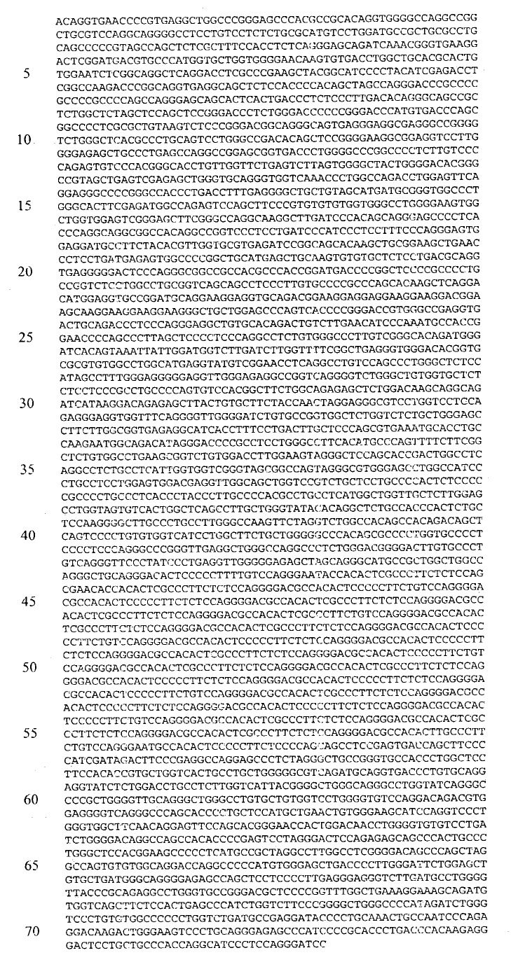

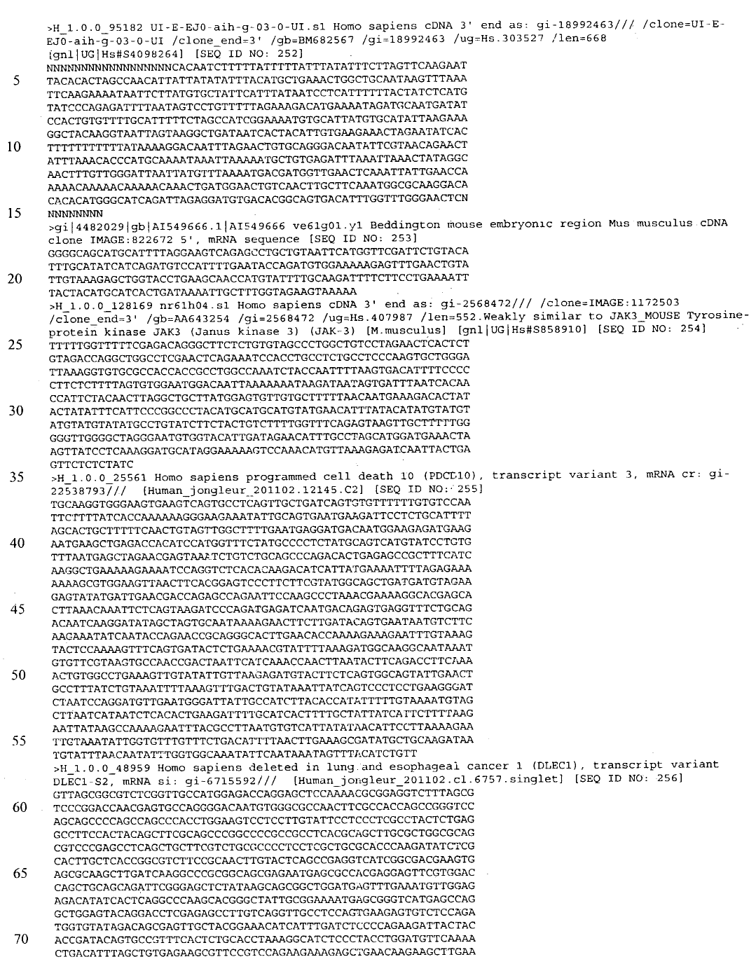

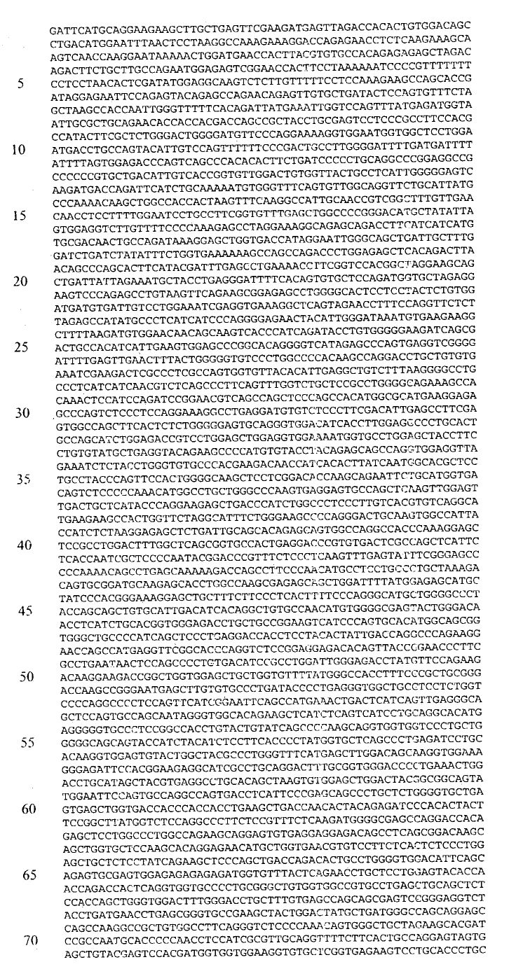

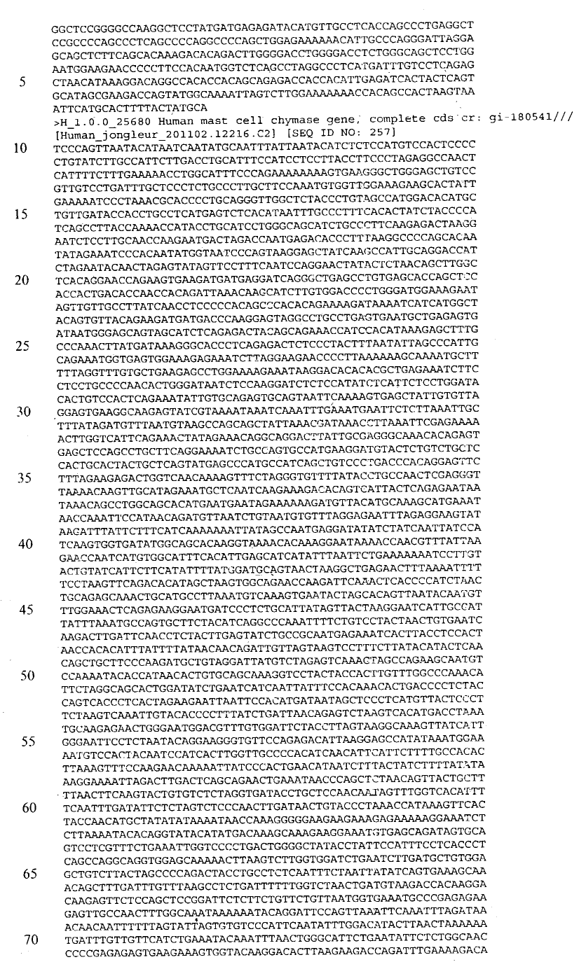

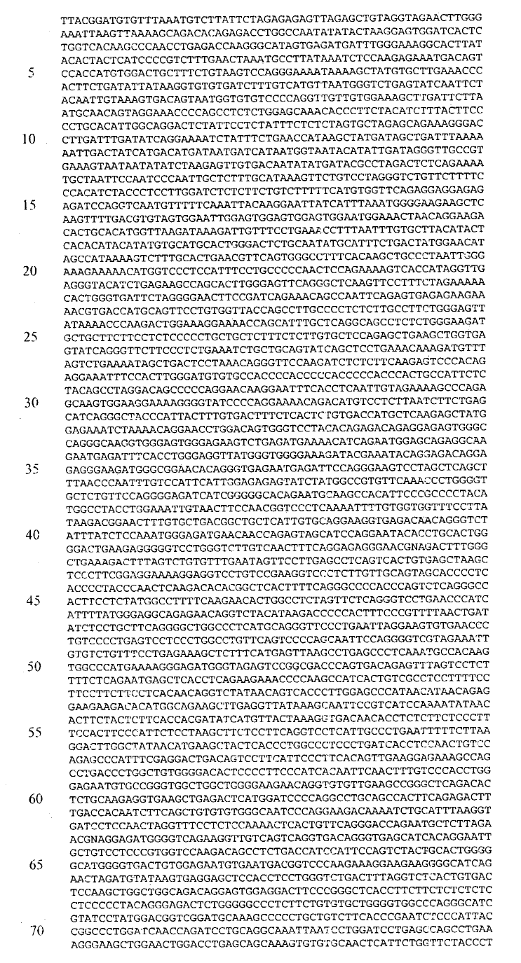

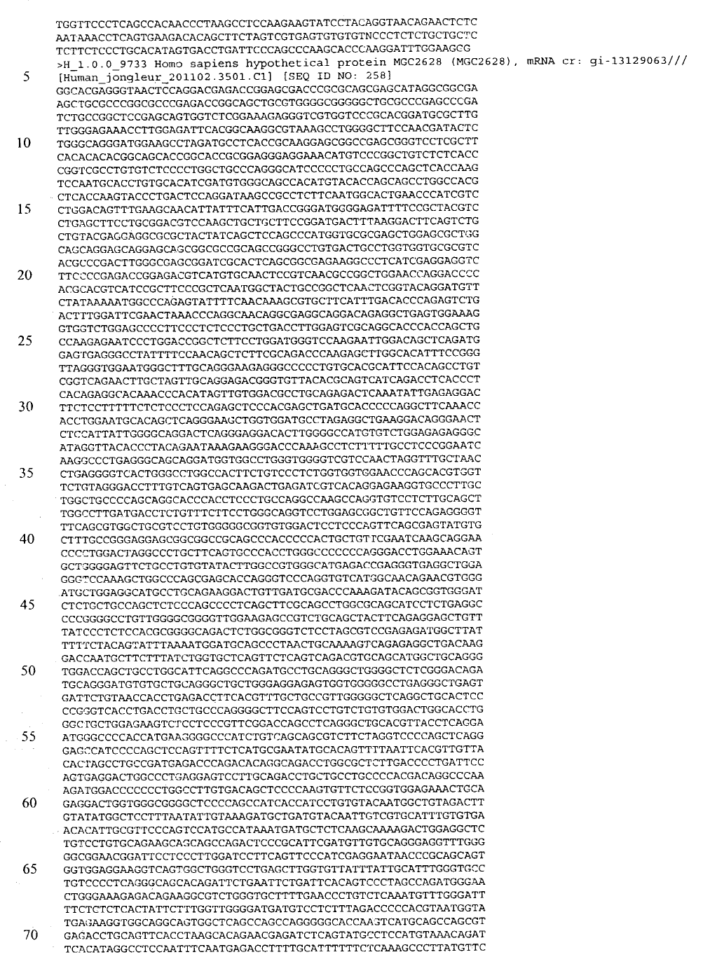

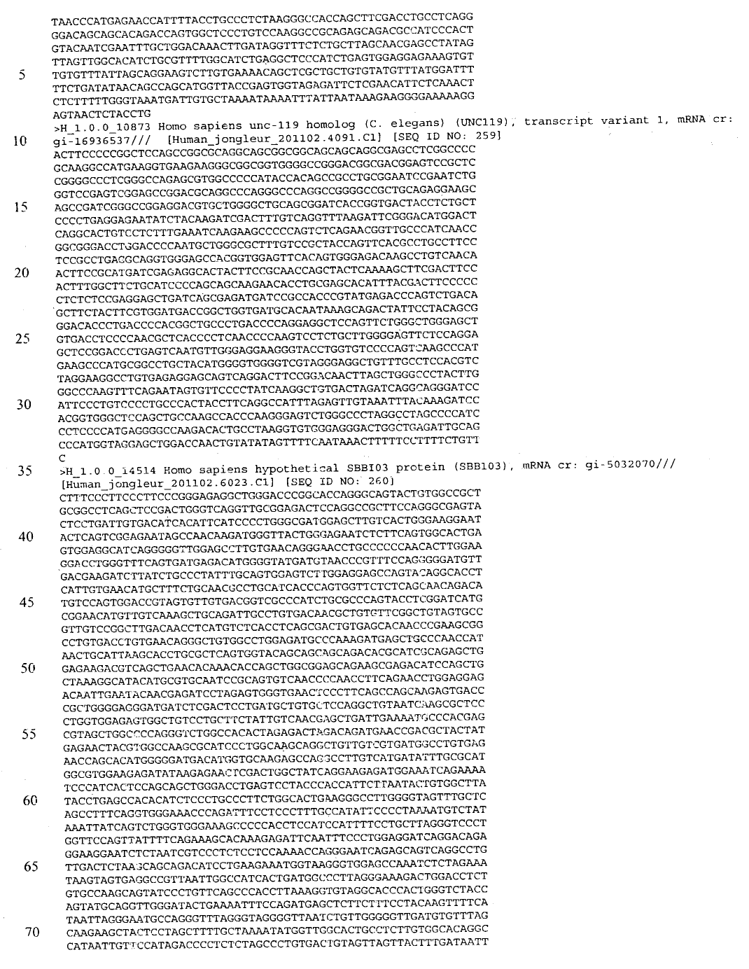

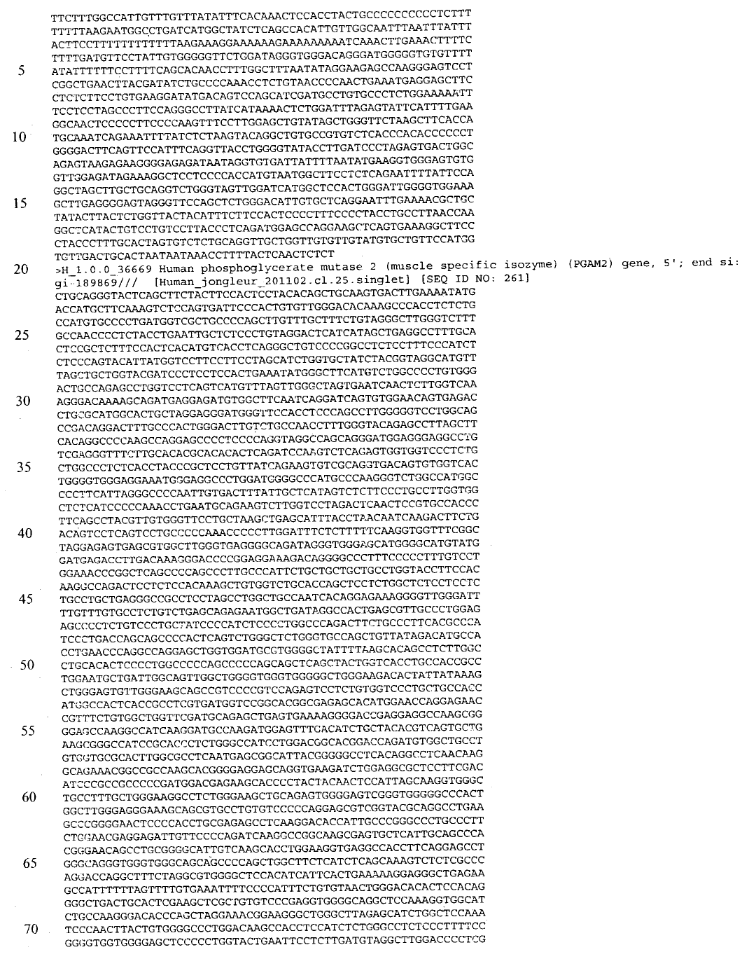

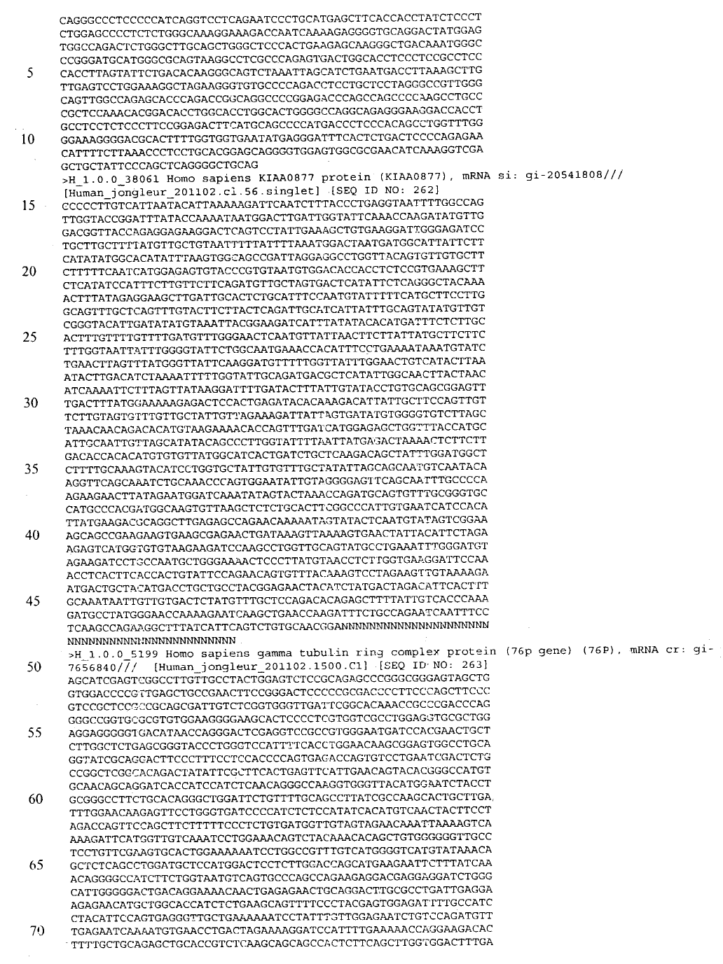

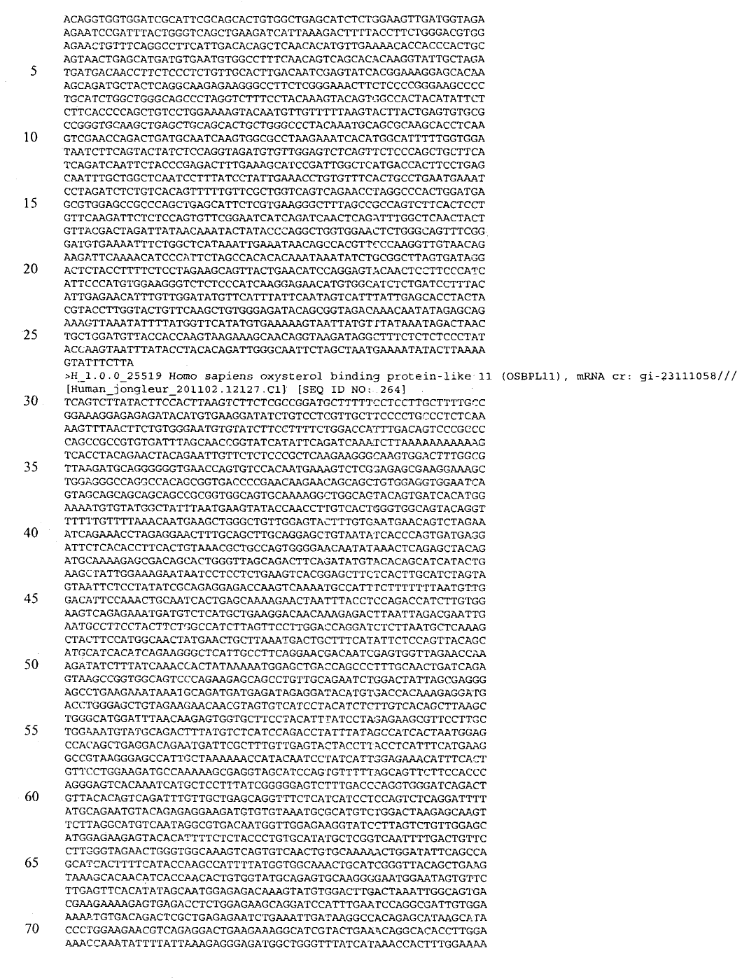

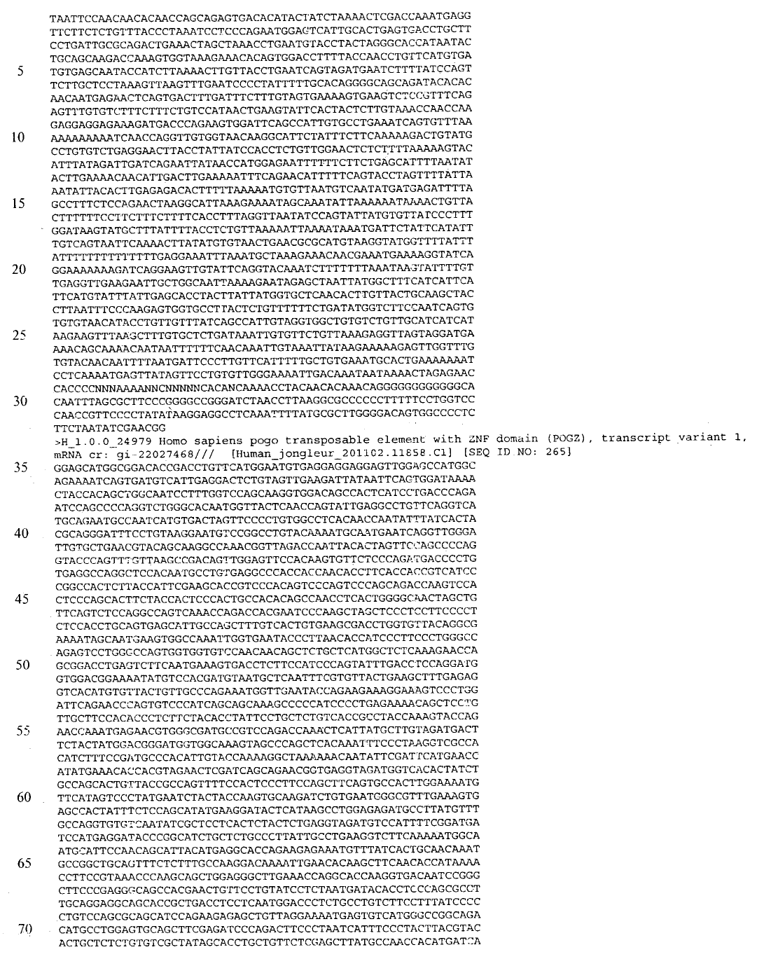

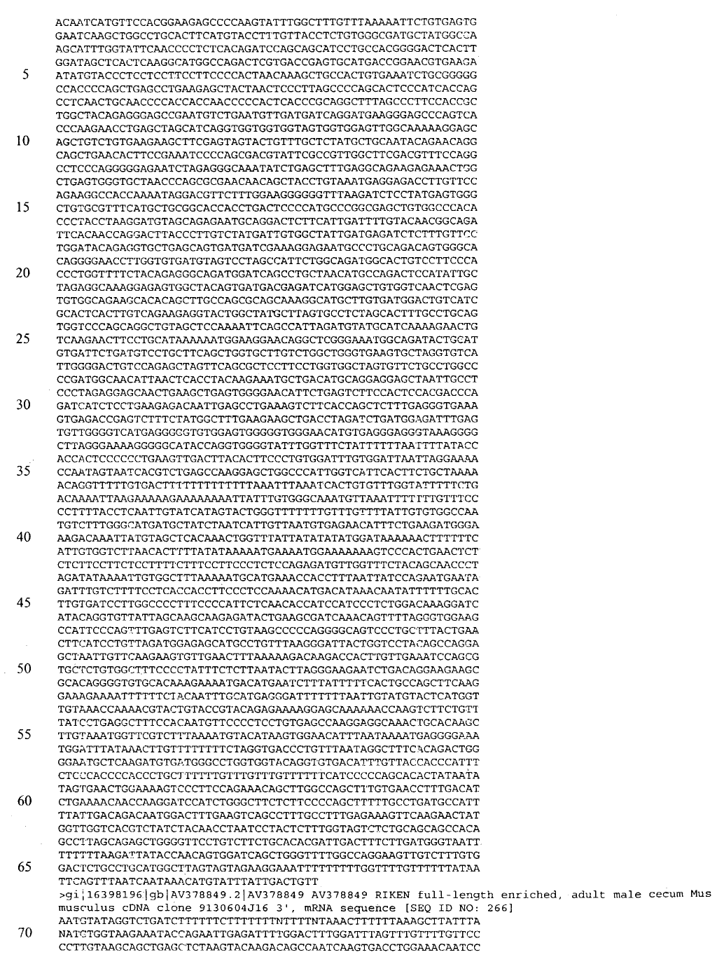

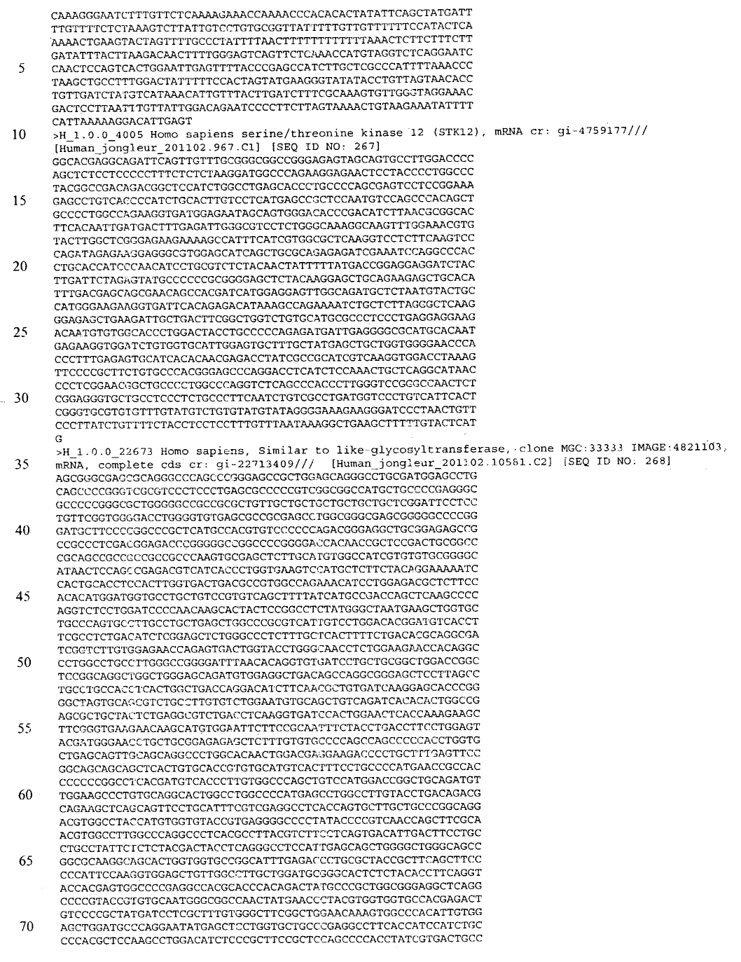

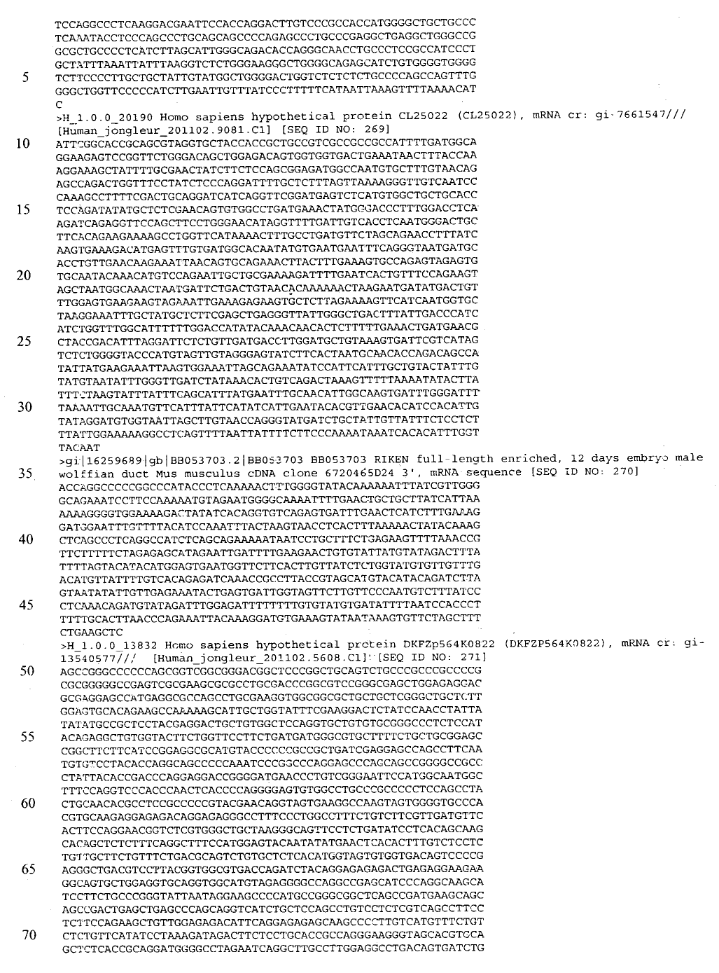

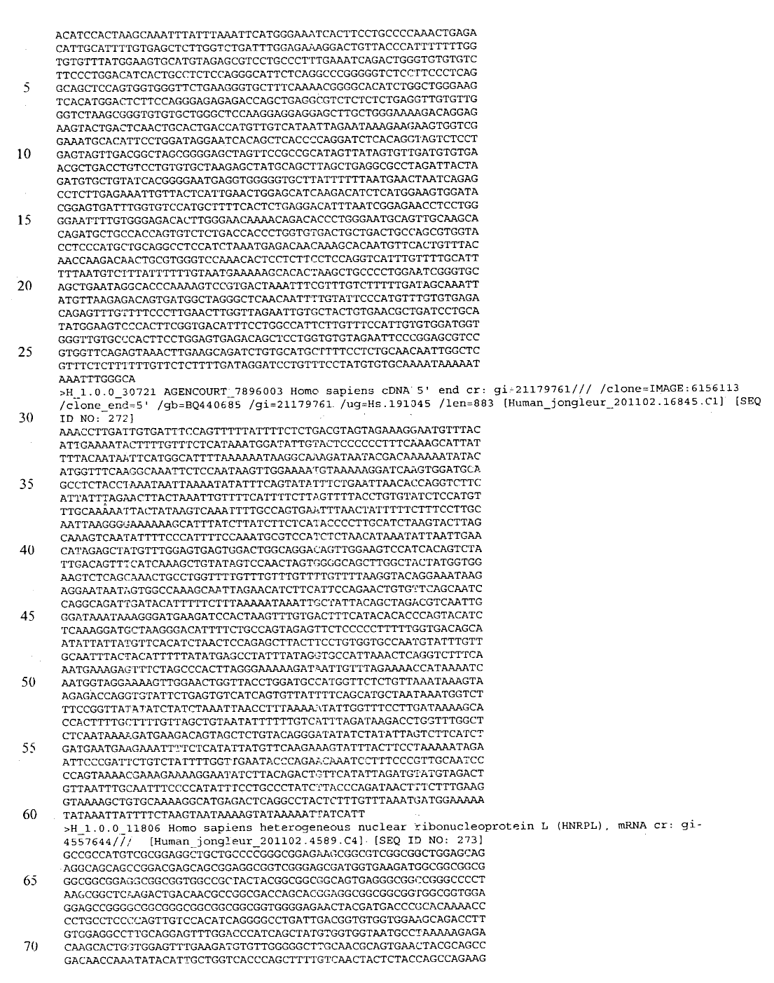

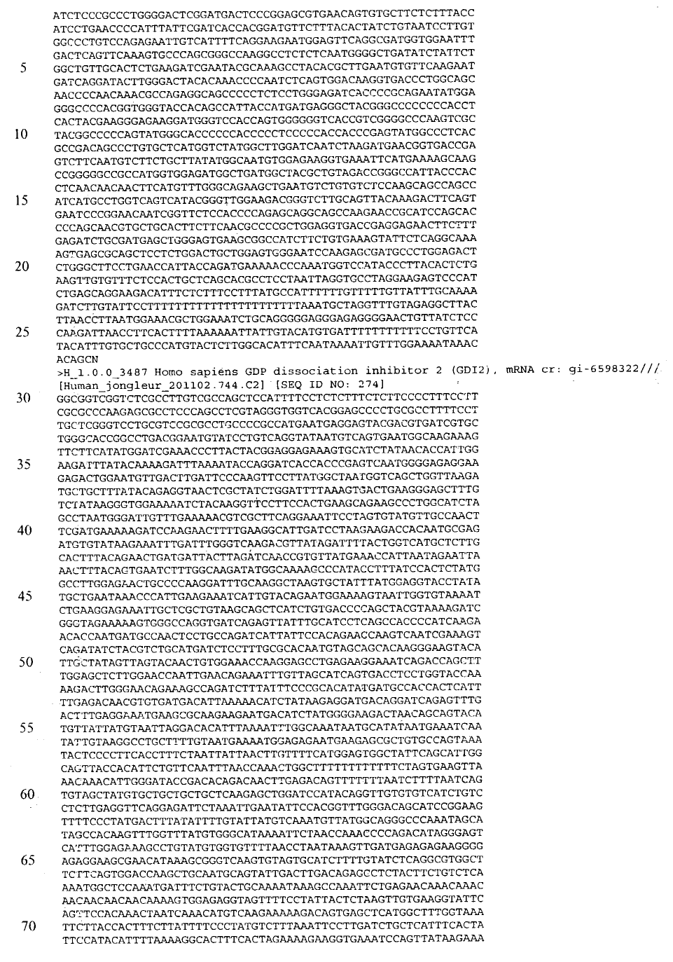

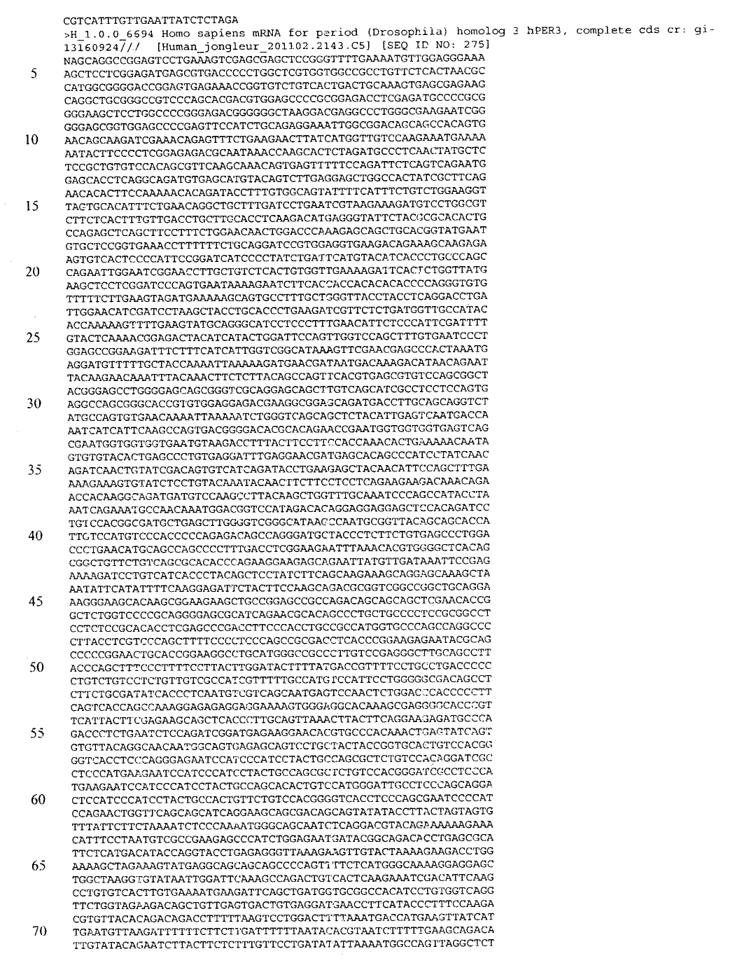

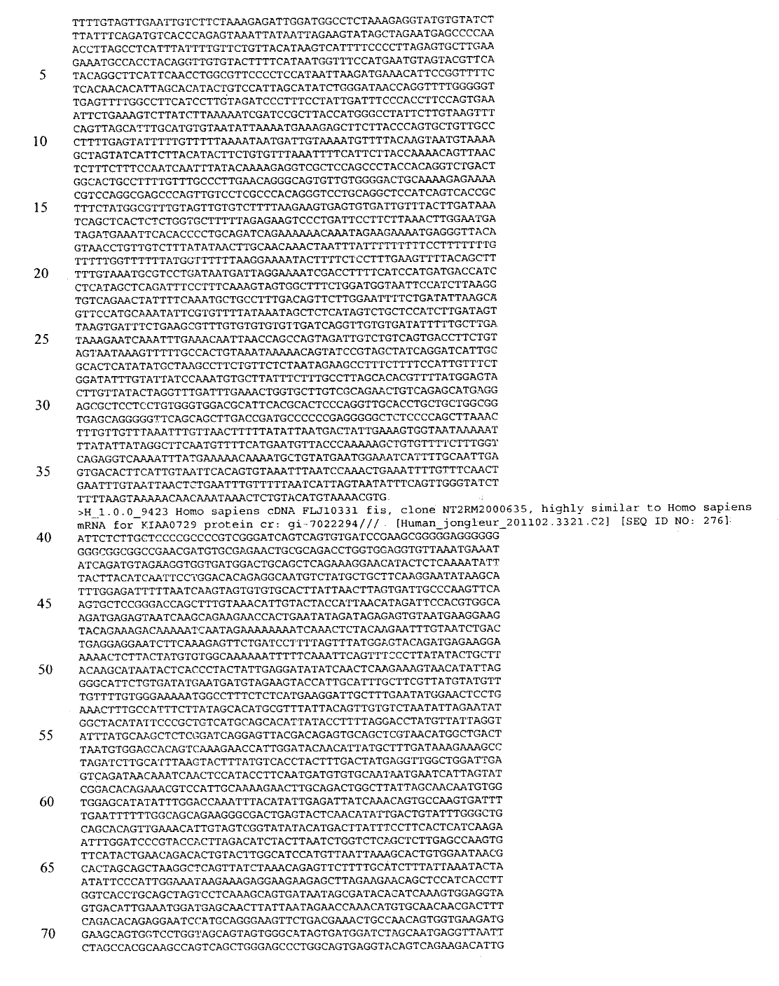

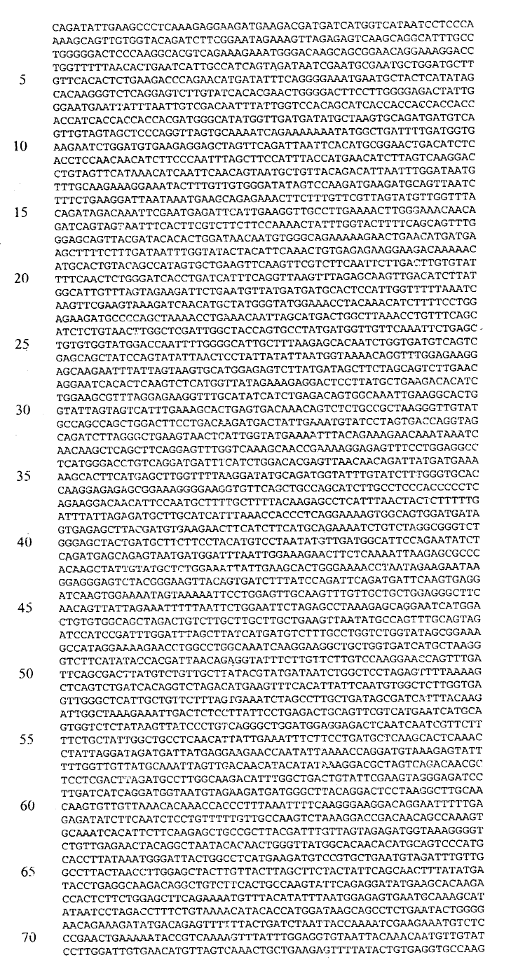

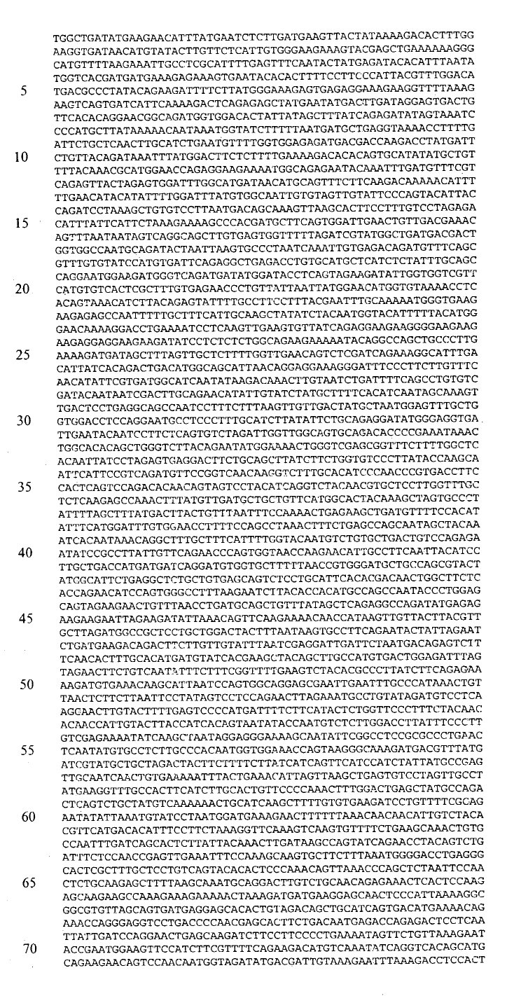

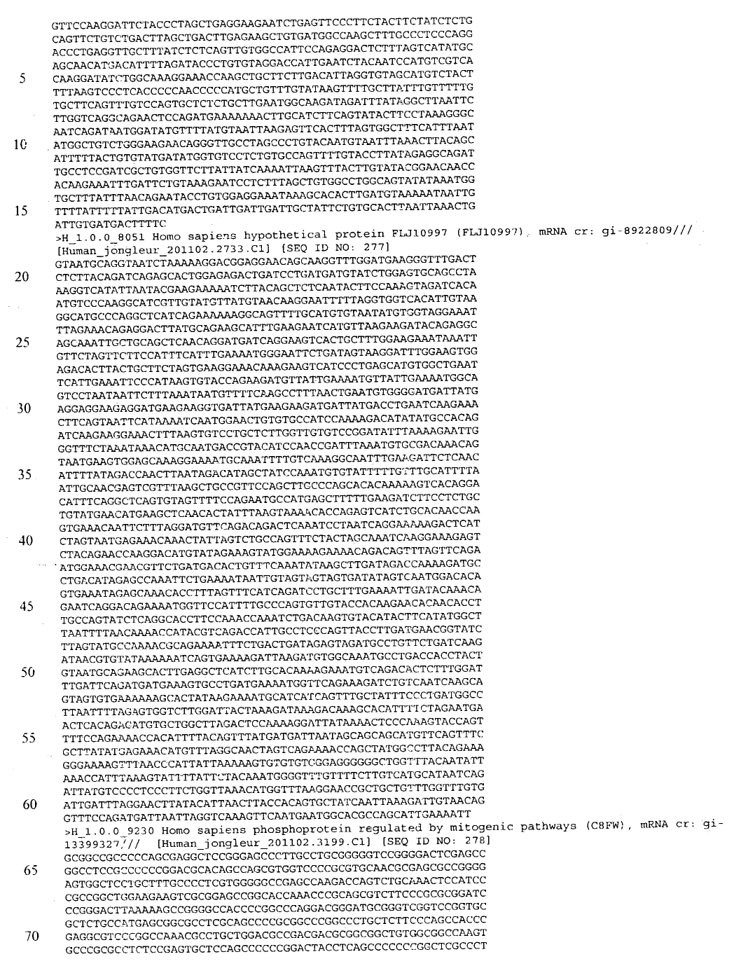

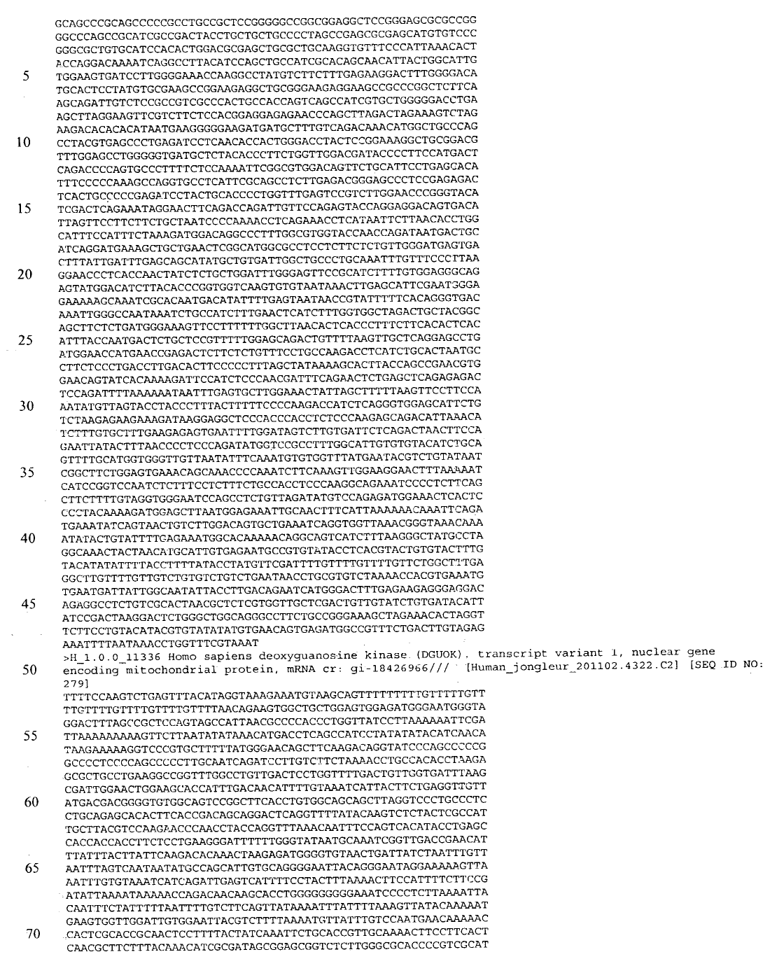

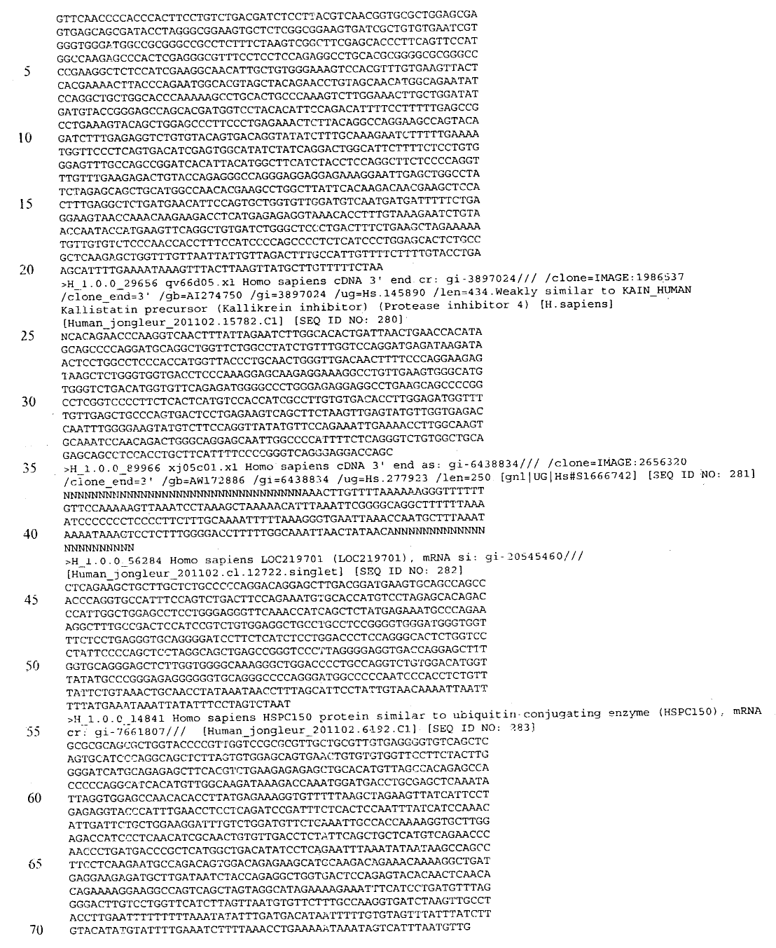

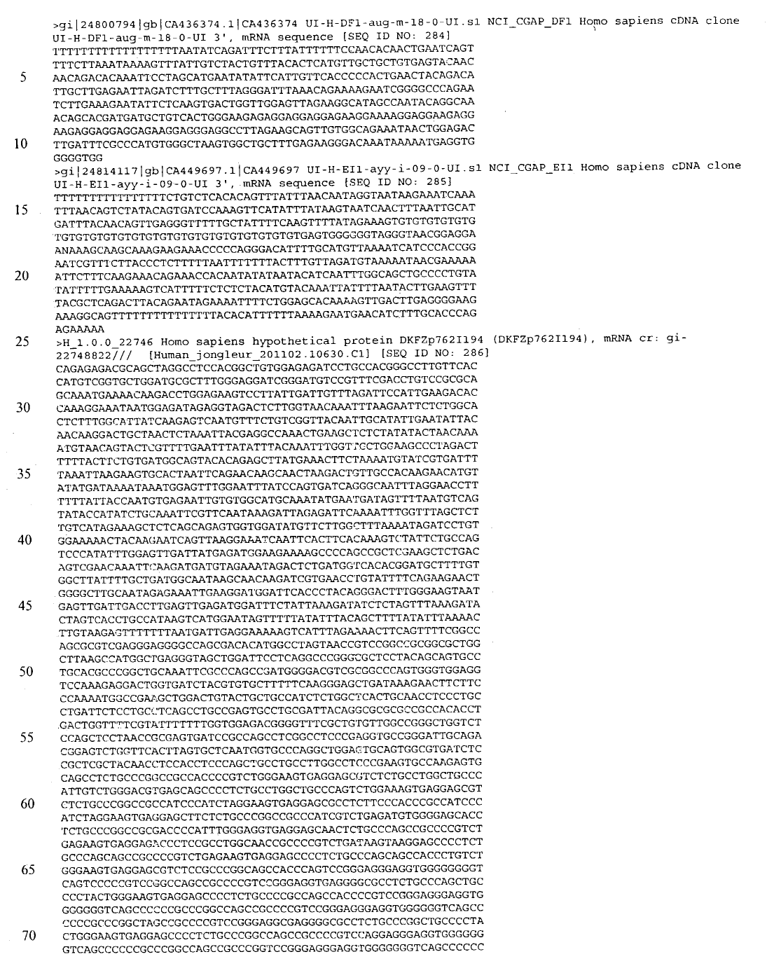

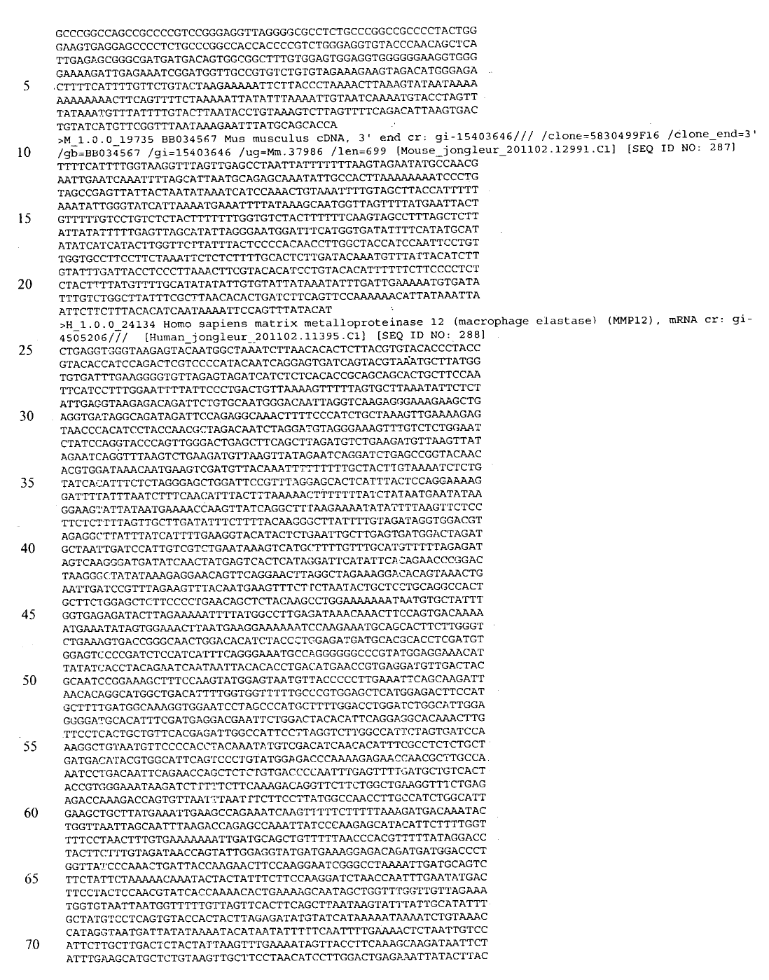

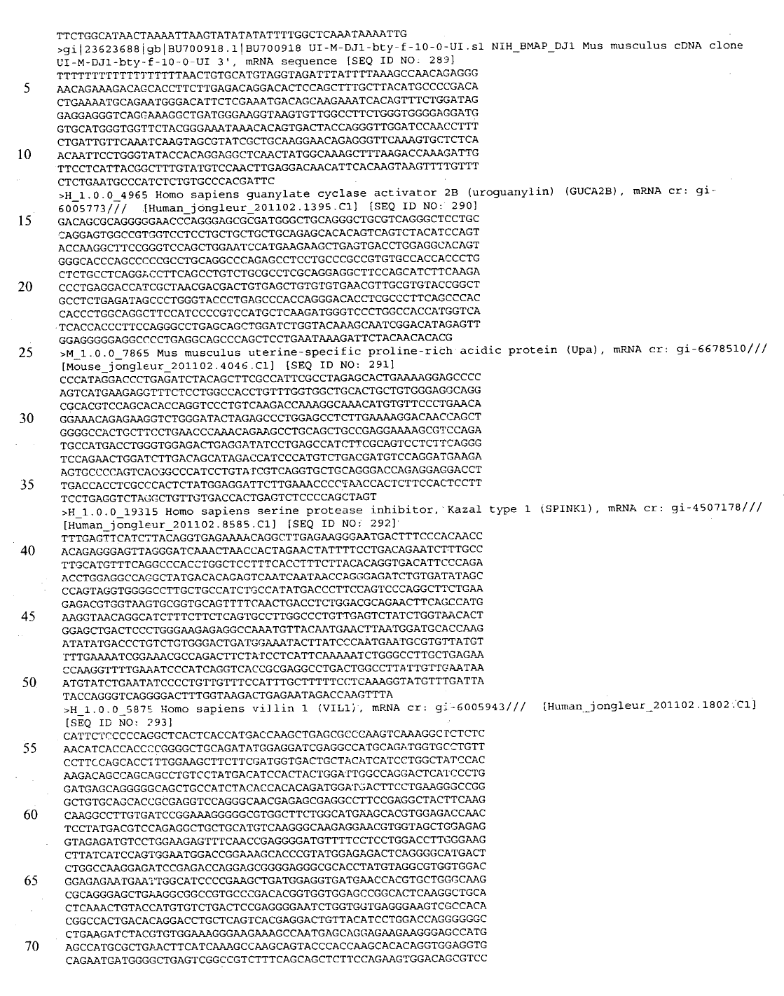

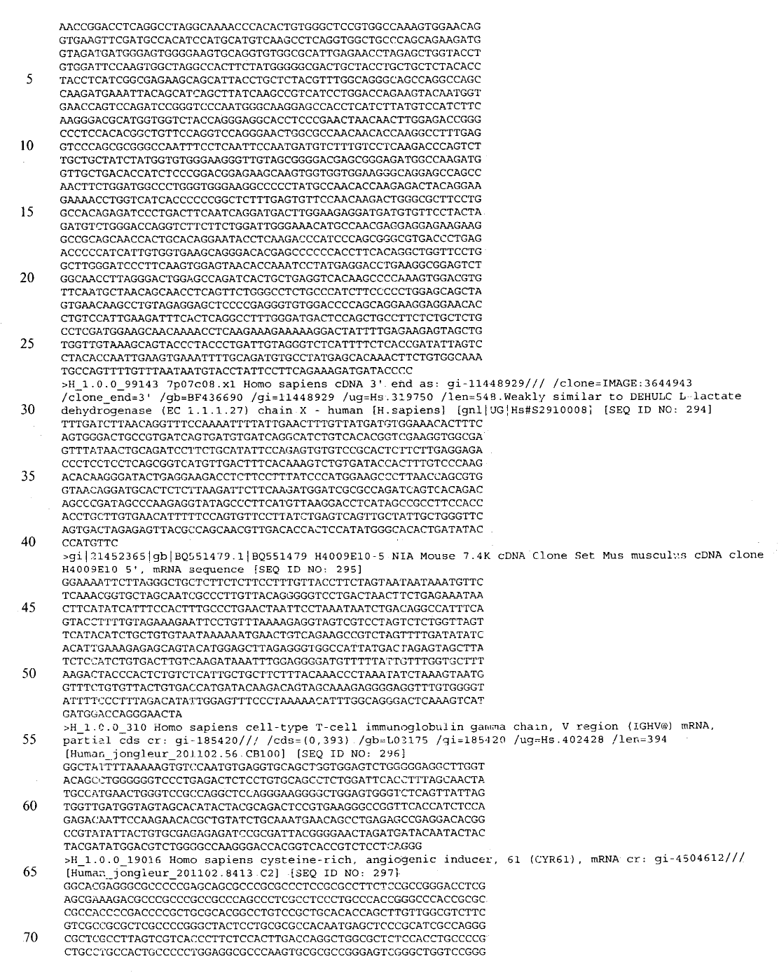

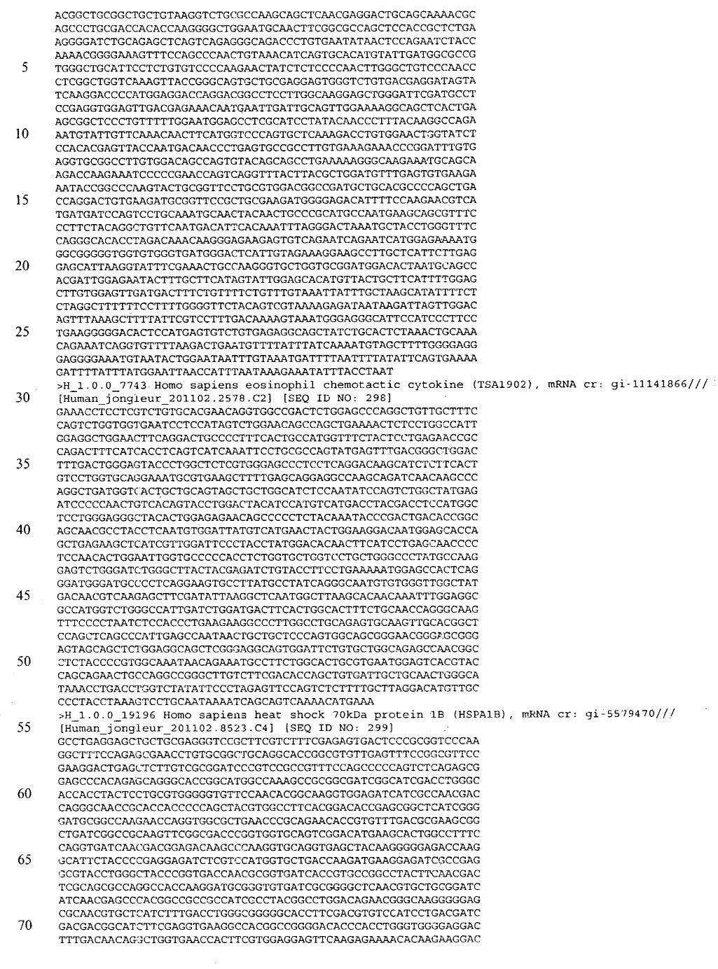

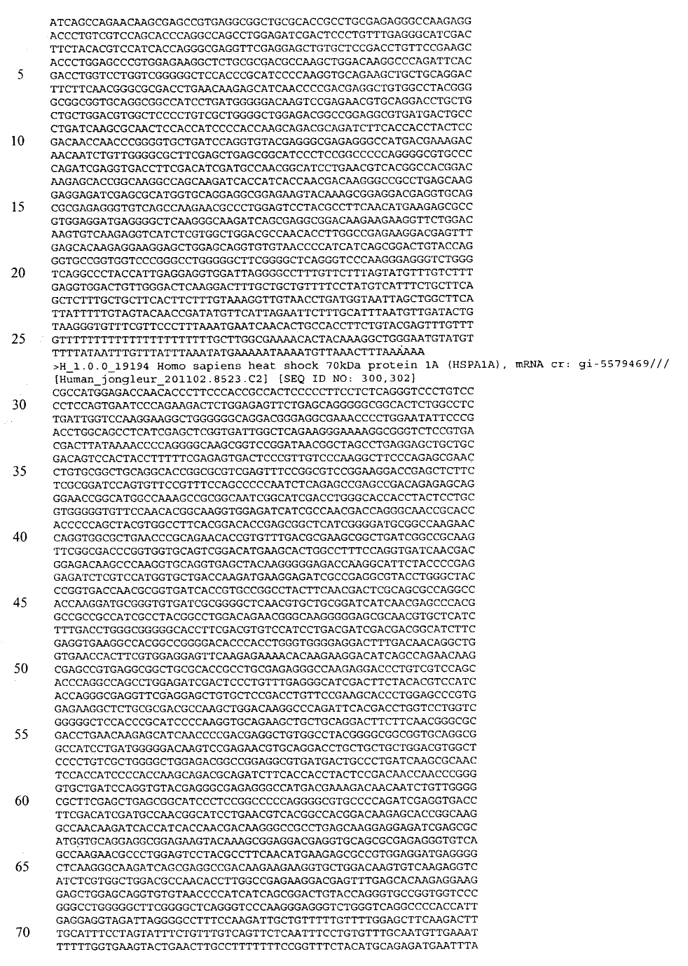

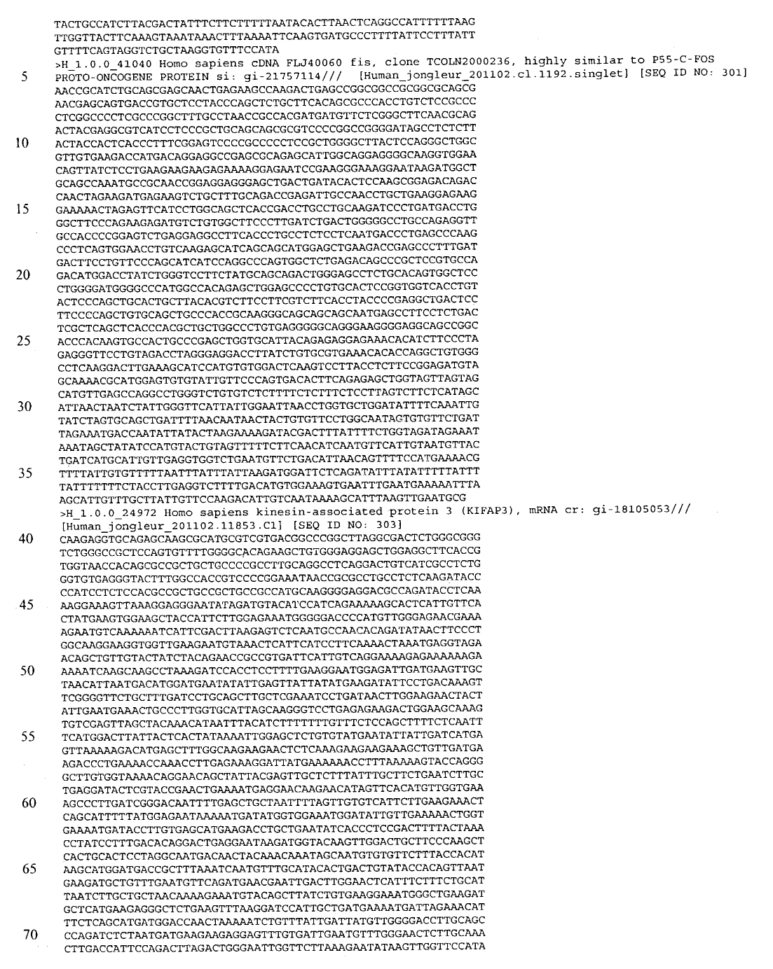

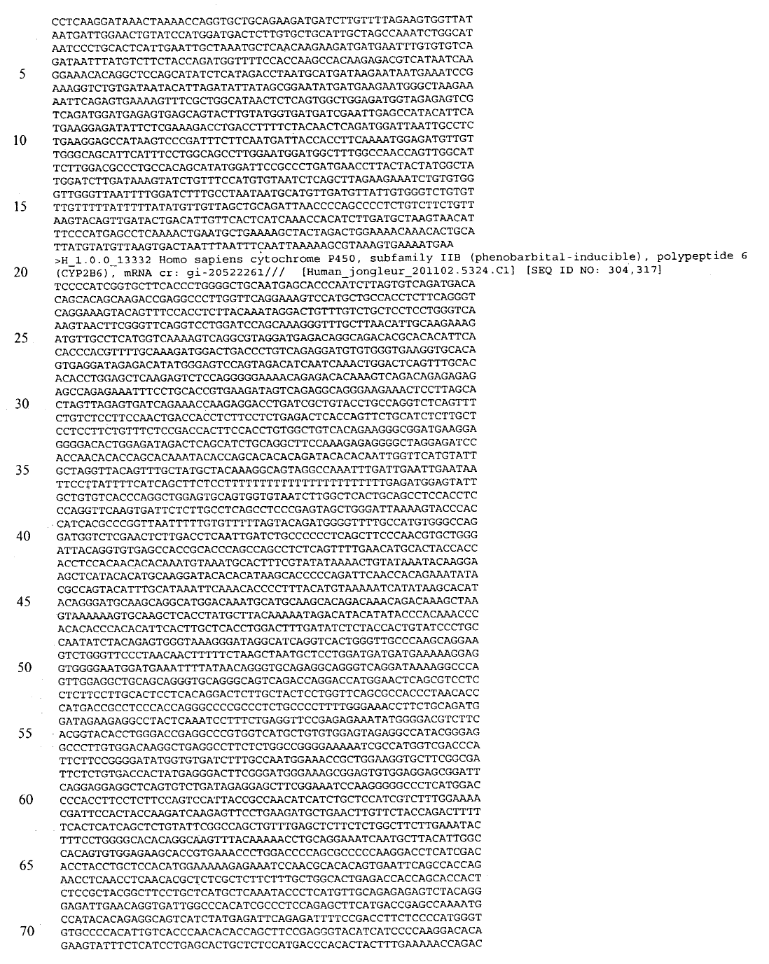

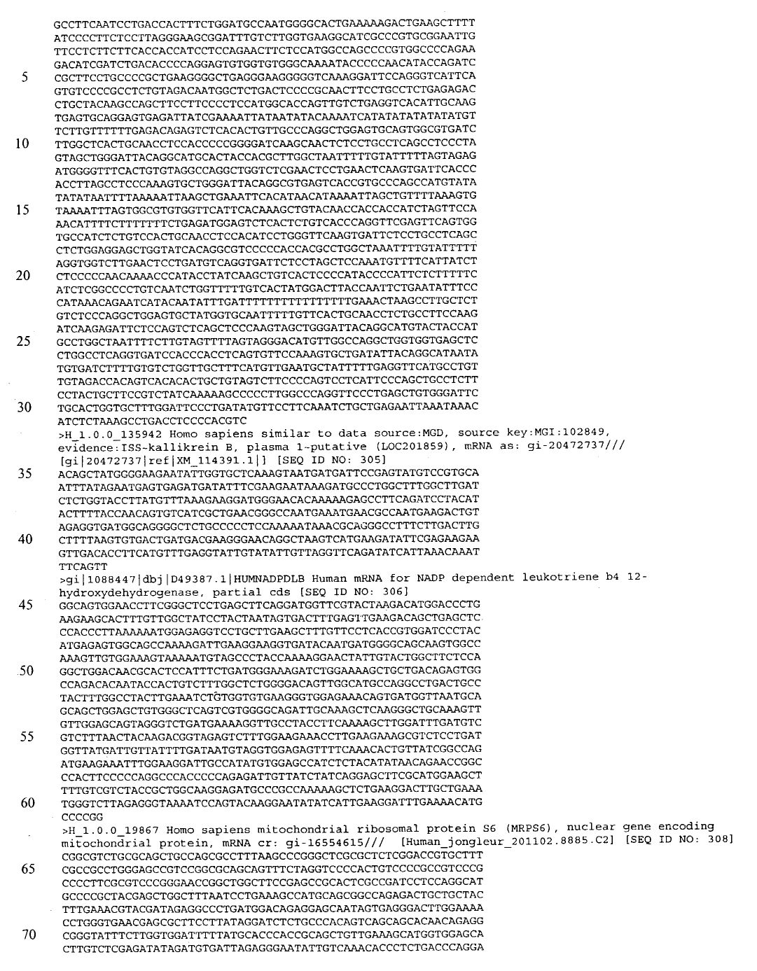

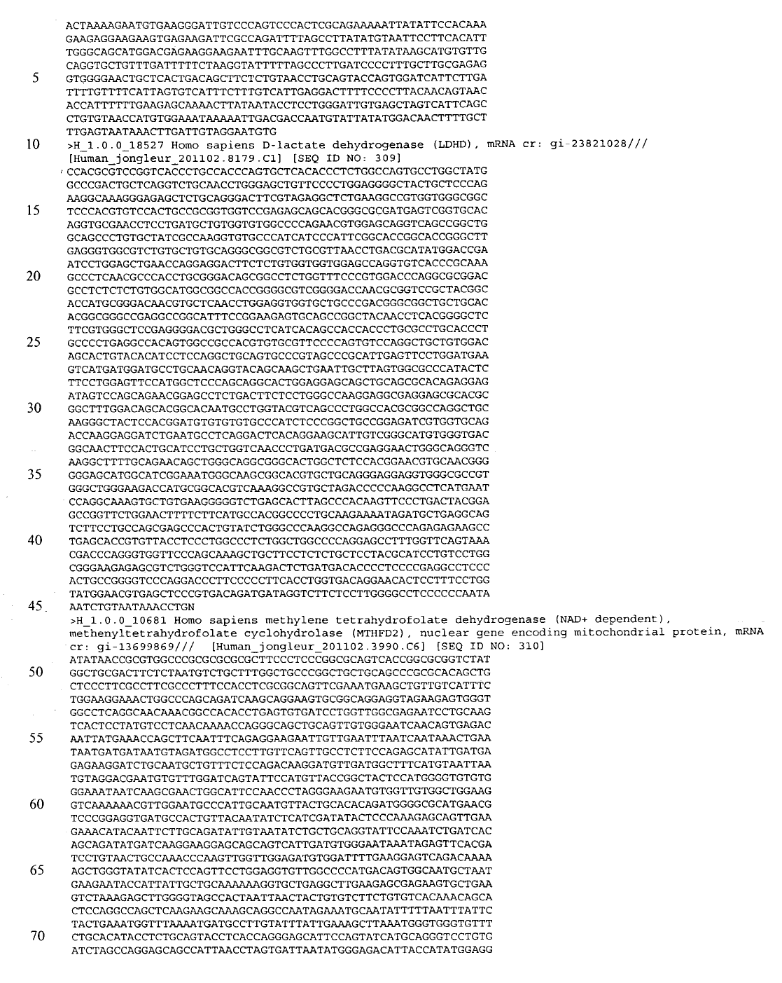

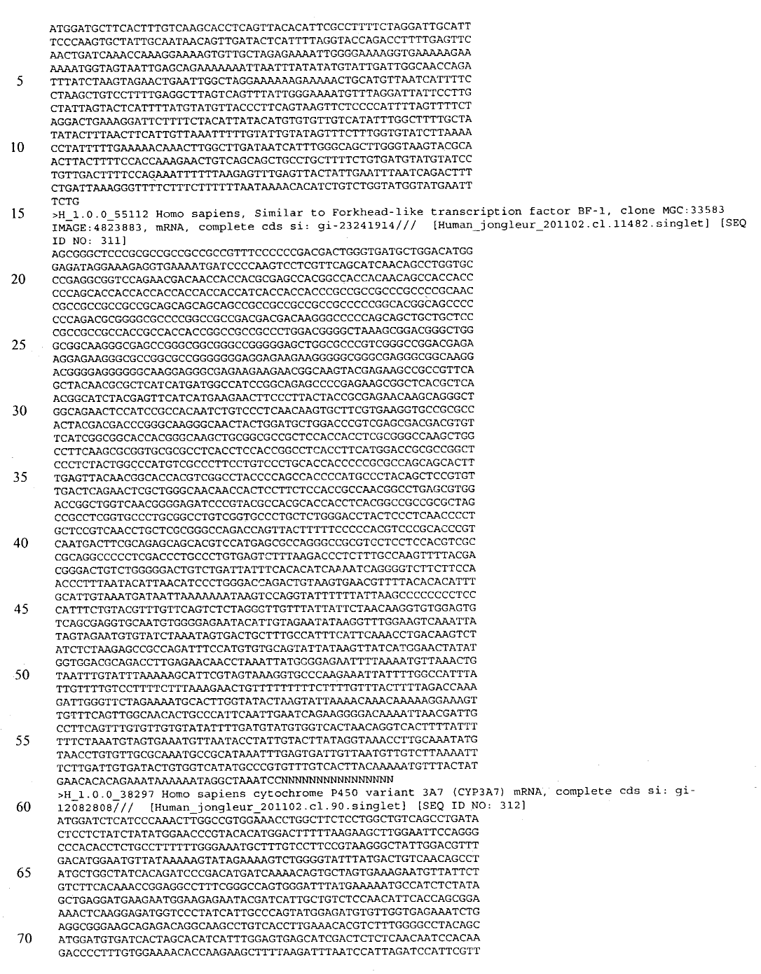

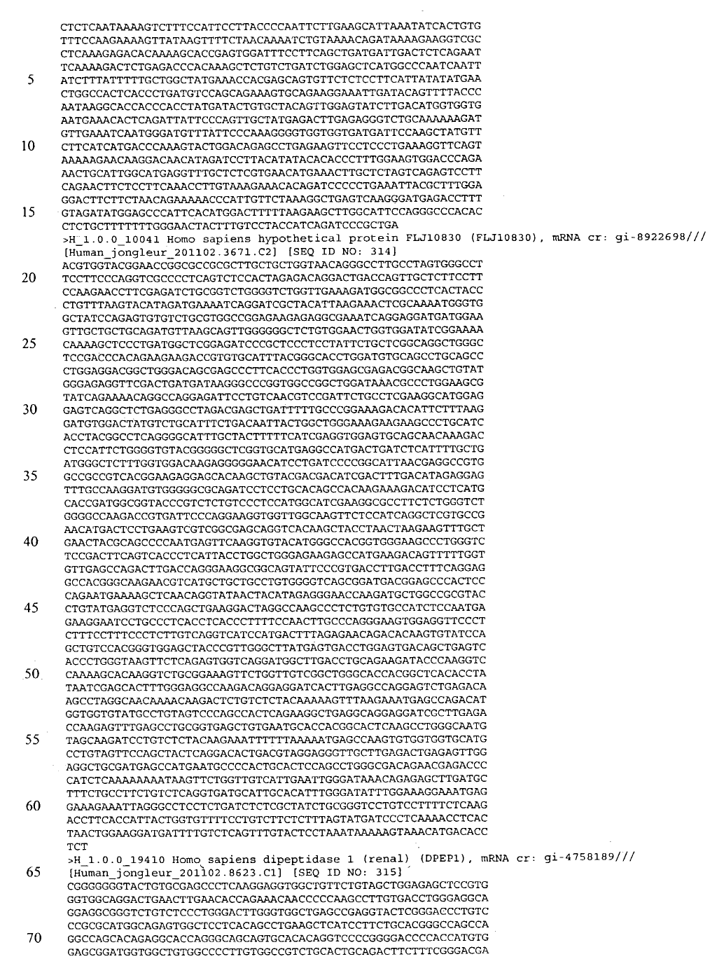

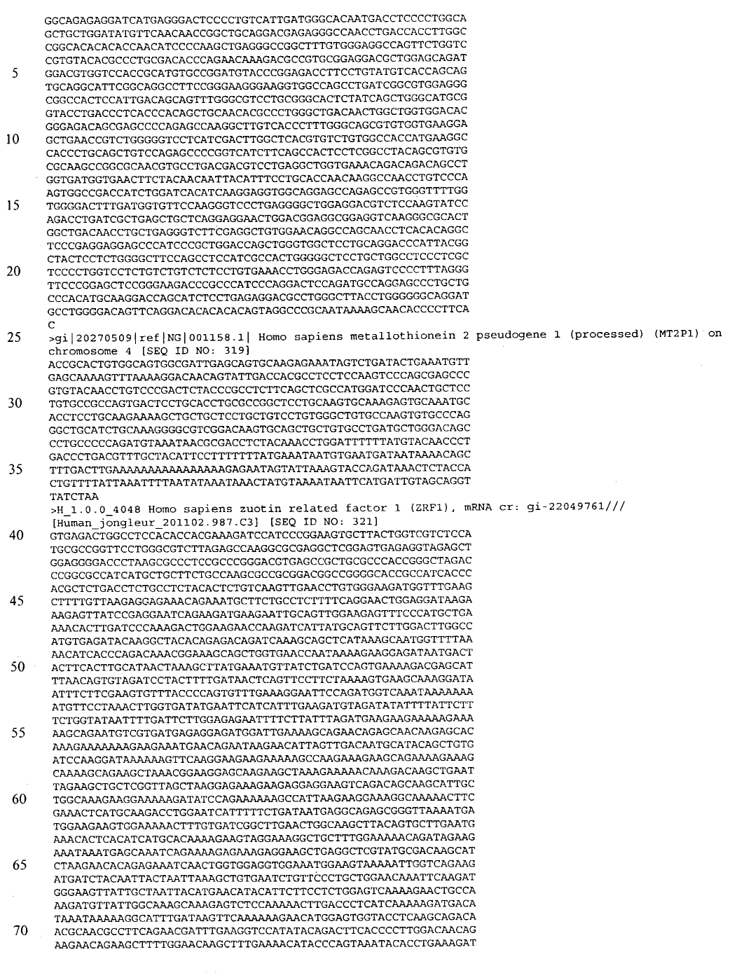

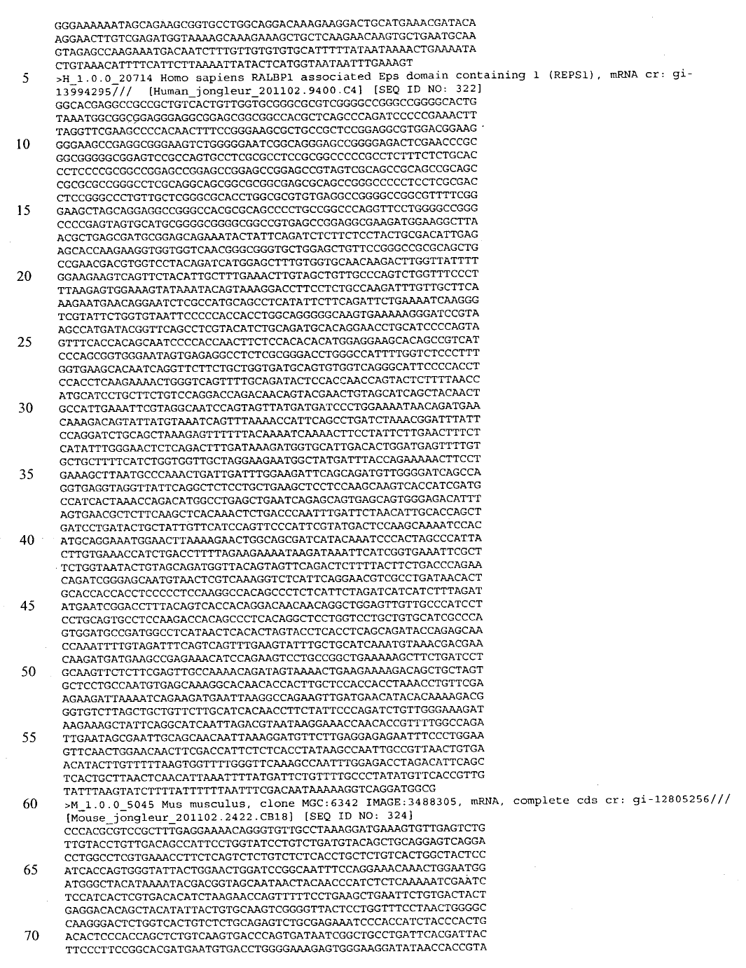

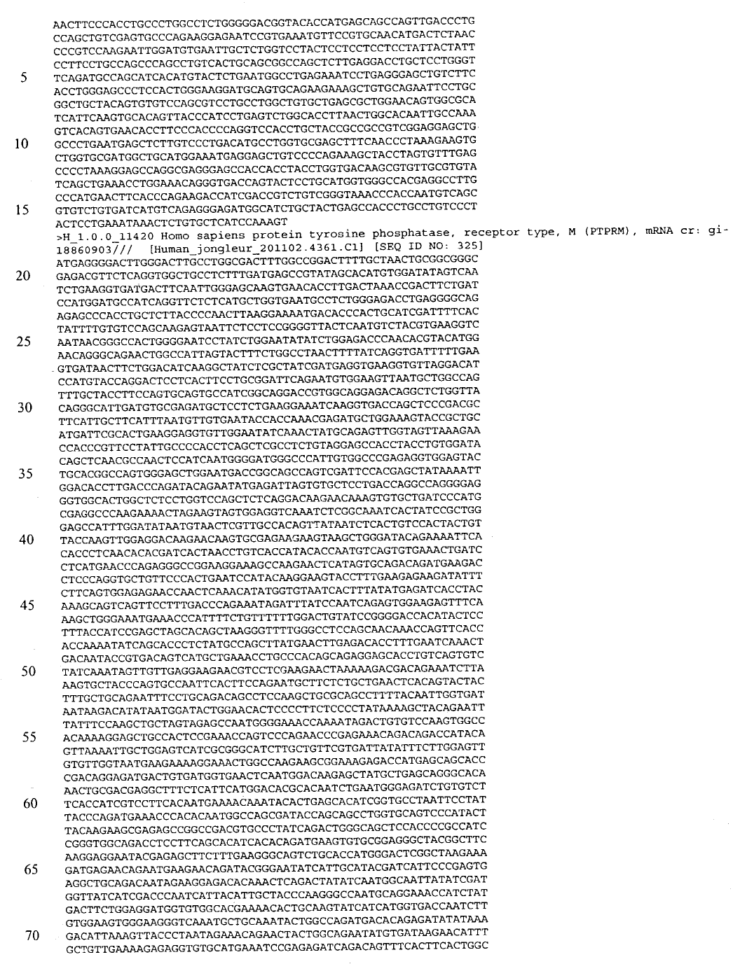

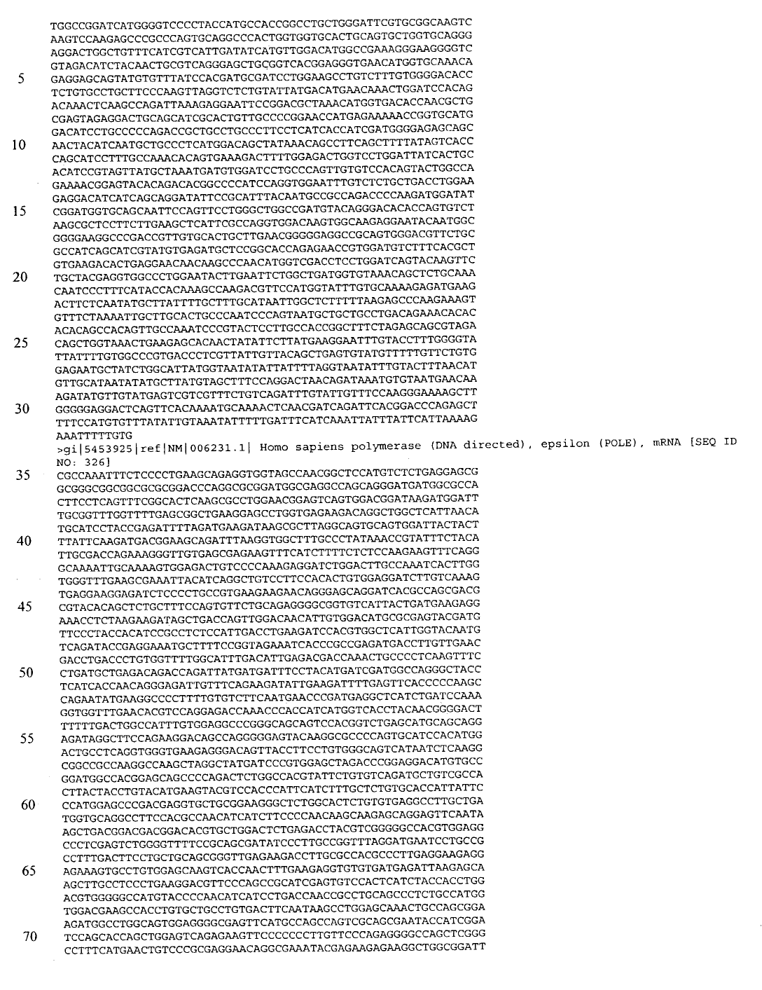

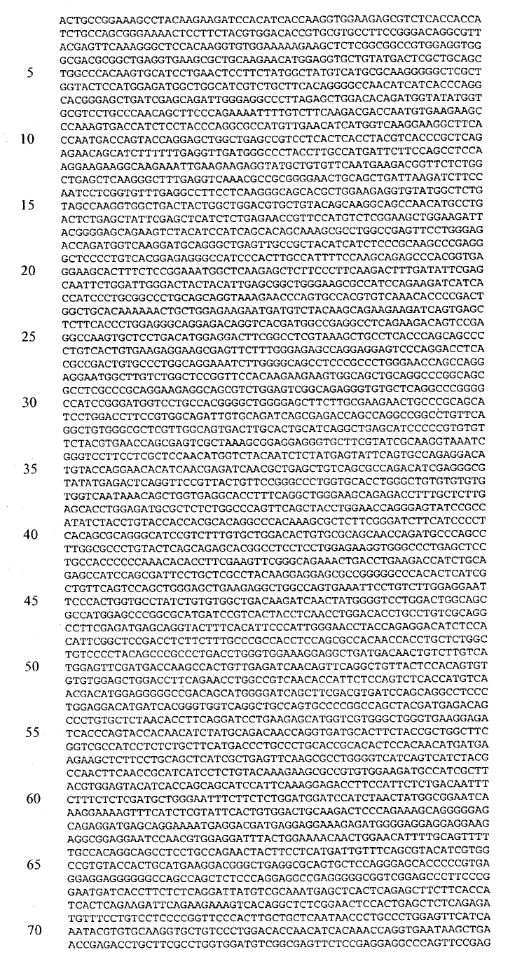

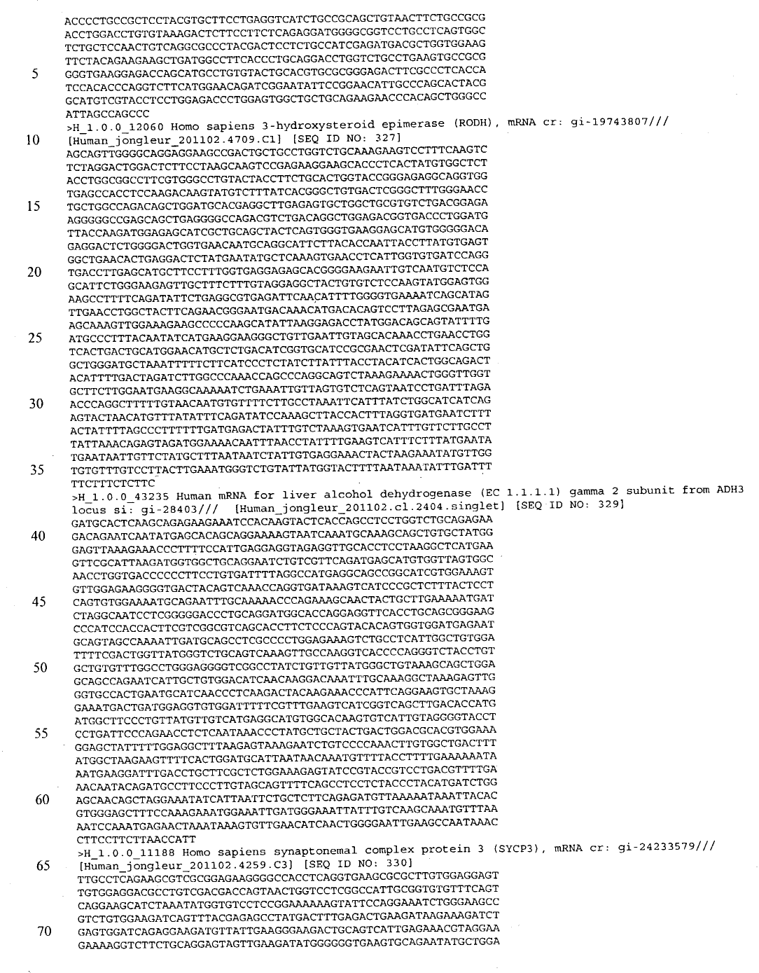

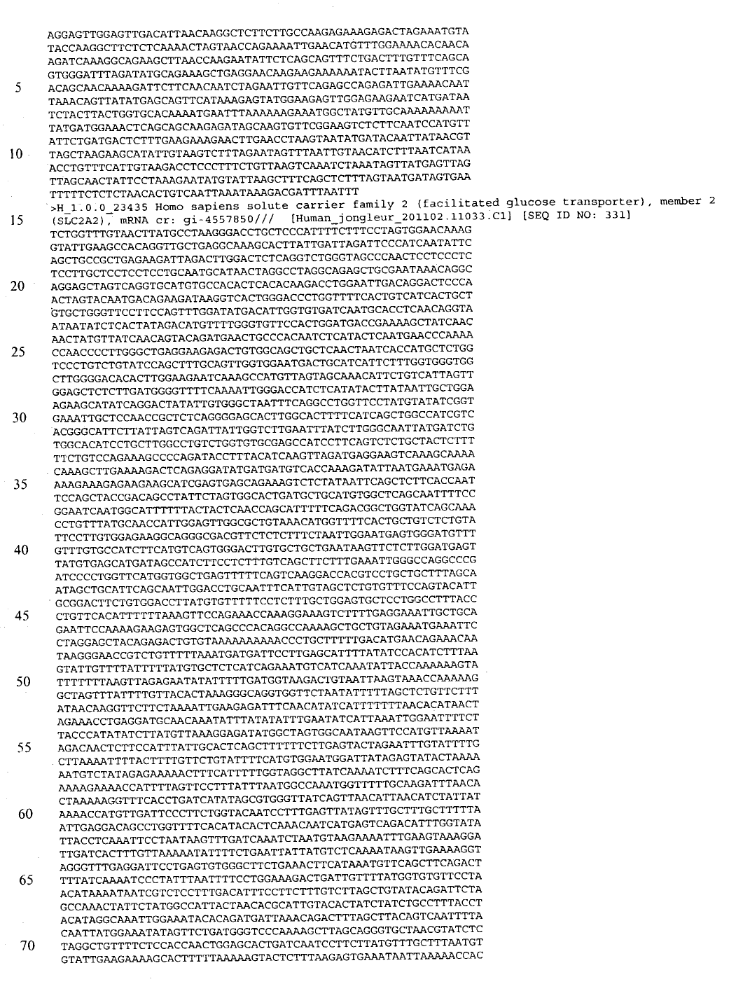

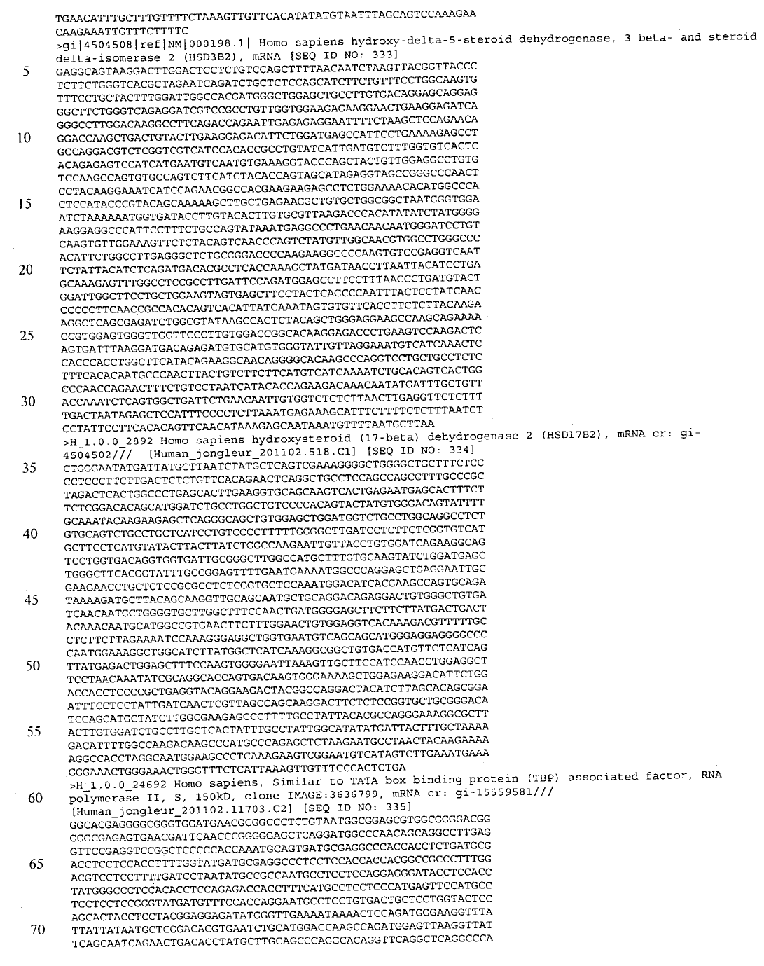

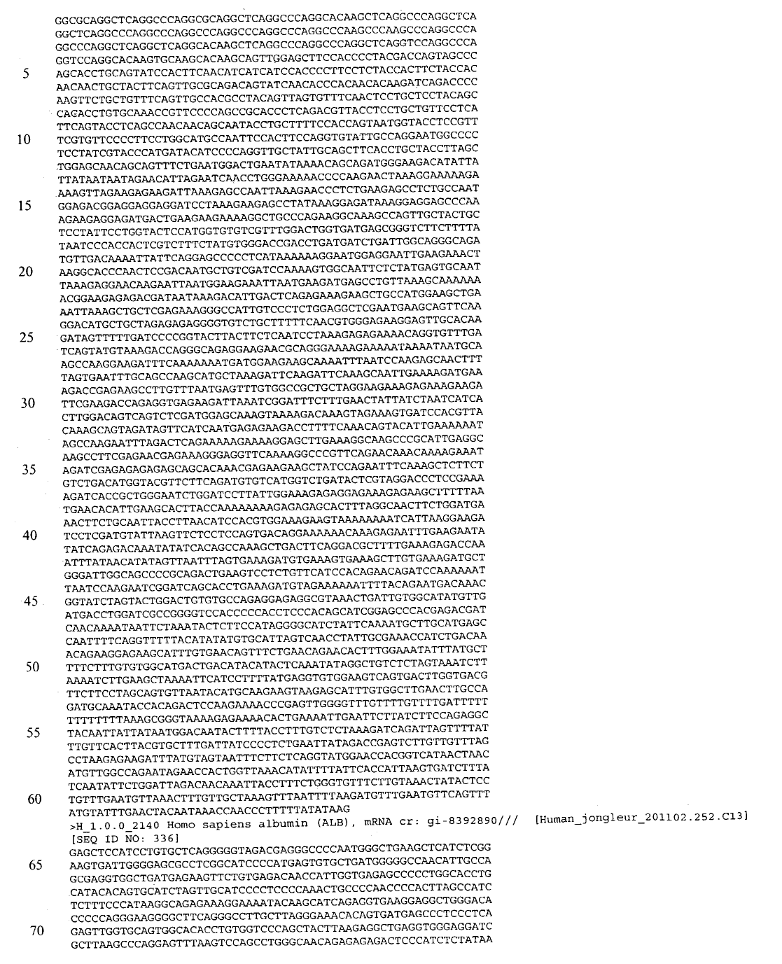

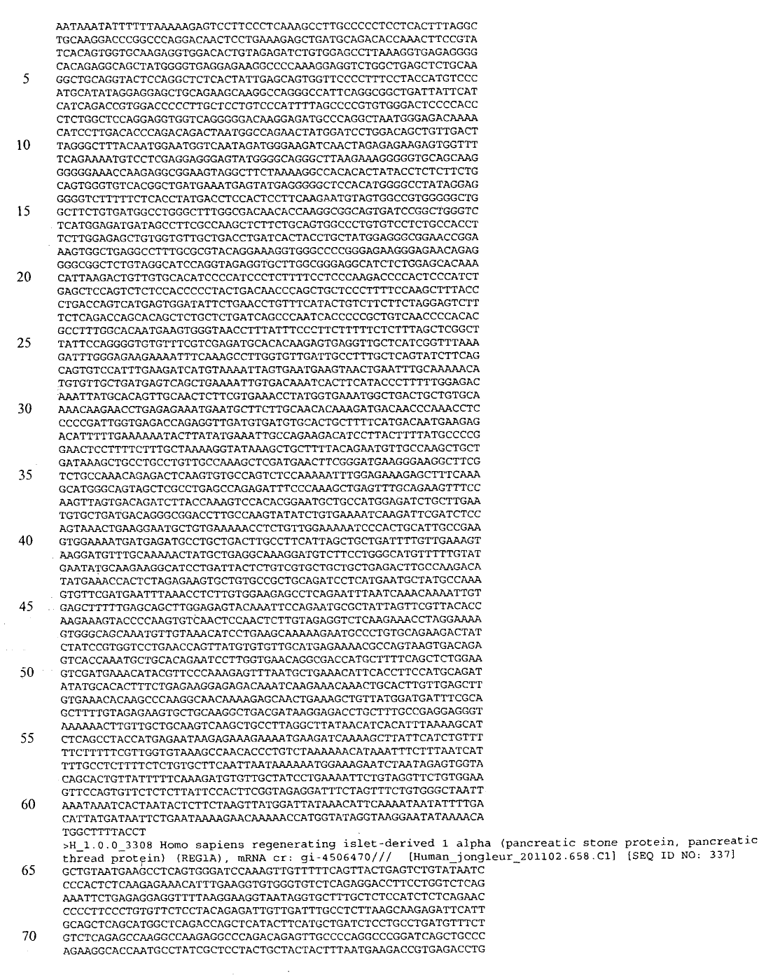

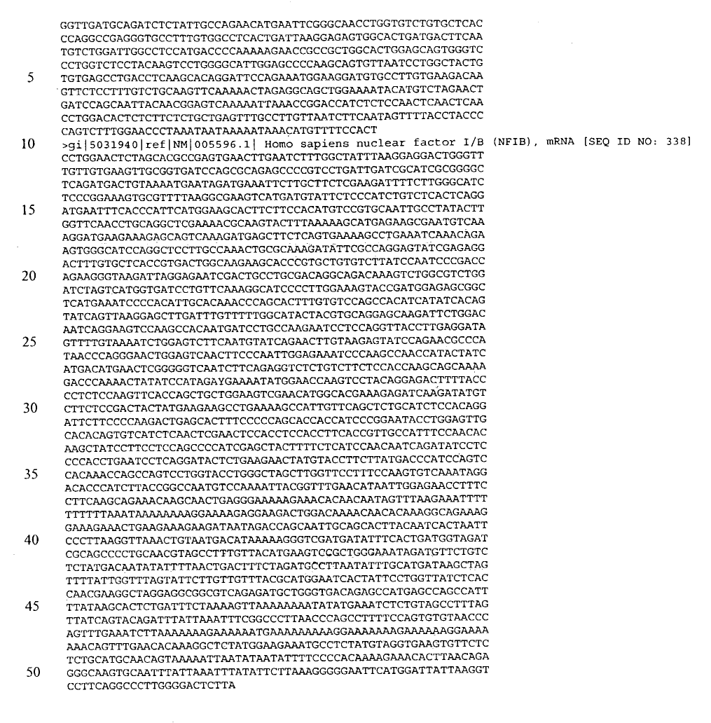

SPLEEN

| 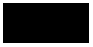 | blad | kidn | pl/ut | mam |
|---|---|---|---|---|
| Description | PL-9B_BDEnor | PL-10B_BDEnor | PL-11B_BDEnor | PL-12B_BDEnor |
| 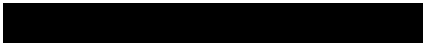 | −4.4 | −1.1 | −1.1 | −1.2 |
| 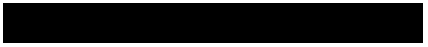 | −1.4 | −1.5 | −1.3 | −2.9 |
| 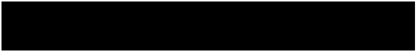 | −1.5 | −1.3 | −1.4 | −2.7 |
| 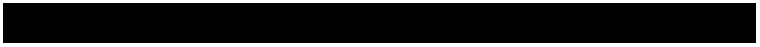 | −2.7 | −1.2 | −1.2 | −1.2 |
| AA982254_RIKEN_cDNA_2210010C04_gene | −2.0 | −1.4 | −1.1 | −1.1 |
| AA675084_elastase_2 | −4.0 | −1.4 | −1.2 | −1.4 |
| AA674409_pancreatic_lipase_related_protein_1 | −1.1 | −1.1 | 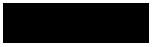 | 1.3 |
| AA222208_surfactant_associated_protein_C | −1.2 | −1.4 | 1.3 | 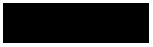 |
| AA445731_heat_shock_protein_cognate_70,_testis | −1.0 | 1.1 | −1.2 | 1.1 |
| AA717025_Mus_musculus_10_day_old_male_pan- | −2.9 | 1.2 | −1.1 | −1.4 |
| creas_cDNA_RIKEN_RIKEN_full- | | | | |
| length_enriched_library,_clone:1810009A17,_full_insert_sequence | | | | |
| AI510089_ESTs | 1.0 | −1.3 | −1.0 | −1.3 |
| W53916_RIKEN_cDNA_3300001P10_gene | 1.2 | 1.0 | 1.1 | 1.1 |
| AA238294_sema_domain,_immunoglobulin_domain_(Ig),_trans- | −1.5 | −1.1 | 1.0 | 1.0 |
| membrane_domain_(TM)_and_short_cyto- | | | | |
| plasmic_domain,_(semaphorin)_G | | | | |
| AA437577_ESTs | −1.8 | −1.5 | −1.4 | −1.5 |
| AA426902_ESTs | −1.6 | −1.5 | −1.3 | −1.4 |
| AA770808_pericentriolar_material_1 | −1.6 | −1.5 | −1.6 | −1.6 |
| AA684320_activin_receptor_interacting_protein_1 | −1.4 | −1.1 | −1.9 | −1.3 |
| AA871914_defensin_related_cryptidin_16 | 1.2 | −1.1 | 1.1 | −1.0 |
| AA546625_ESTs,_Weakly_simi- | −1.7 | −1.7 | −1.4 | −1.6 |
| lar_to_SP62_MOUSE_SPLICESOME_AS- | | | | |
| SOCIATED_PROTEIN_62_[M.musculus] | | | | |
| AI464778_ESTs | −1.7 | −1.6 | −1.5 | −1.6 |
| AA822098_apolipoprotein_A-I | −1.2 | −2.1 | −2.5 | −1.6 |
| AI325332_transglutaminase_3,_E_polypeptide | −1.7 | −1.4 | −1.5 | −1.7 |
| AA822366_inhibitor_of_DNA_binding_4 | −1.1 | −1.5 | −1.6 | −1.2 |
| AA733740_ESTs | −1.7 | −1.8 | −1.7 | −1.8 |
| AA881492_ESTs | −1.5 | −1.5 | −1.7 | −1.1 |
| AI608086_runt_related_transcription_factor_3 | −1.6 | −1.3 | −1.9 | −1.0 |
| AI121690_RIKEN_cDNA_1300007C21_gene | −1.7 | −1.5 | 1.1 | 1.2 |
| AI385457_retinol_binding_protein_2,_cellular | −1.6 | −1.9 | −1.5 | −1.7 |
| AA255025_S100_calcium-binding_protein_A9_(calgranulin_B) | 1.1 | −1.1 | 1.1 | −1.6 |
| AI551662_fos-like_antigen_2 | −1.4 | −1.4 | −1.4 | −1.5 |
| AA414790_leukotriene_A4_hydrolase | −2.0 | −1.7 | −1.6 | −1.8 |
| AA014727_tumor-suppressing_subchromosomal_transferable_frag- | −2.1 | −1.8 | −2.9 | −1.0 |
| ment_3 | | | | |
| AA546645_cell_division_cycle_2_homolog_(S._prombe)-like_2 | −2.3 | −1.5 | −2.4 | −1.4 |
| AA444538_ESTs,_Weakly_similar_to_MDP1_MOUSE_MICRO- | 1.3 | −1.3 | −1.3 | 1.3 |
| SOMAL_DIPEPTIDE_PRECURSOR_[M.musculus] | | | | |
| AA608466_ESTs | −1.3 | −1.2 | −1.1 | −1.1 |
| AI386257_calbindin-D9K | −1.1 | −1.2 | −1.2 | 1.4 |
| AA087193_lipocalin_2 | 1.2 | −2.02682* | −1.6 | 1.1 |
| AA034678_rhotekin | −2.1 | −1.4 | −2.6 | −1.4 |
| AA414653_ESTs,_Weakly_similar_to_KIAA0542_protein_[H.sapiens] | −1.3 | −1.1 | −1.29631+ | −1.1 |
| AA771229_ESTs,_Weakly_similar_to_zinc_finger_pro- | −2.2 | −1.4 | −2.4 | −1.4 |
| tein_95_[M.musculus] | | | | |
| AA982151_proteinase_3 | −1.2 | −1.2 | −1.1 | 1.1 |
| AA176045_forkhead_box_C2 | −2.3 | −1.6 | −2.5 | −1.1 |
| AA024217_Public_domain_EST | −1.9 | −1.2 | −2.3 | 1.1 |
| AI428661_ESTs | −2.1 | −1.7 | −2.2 | −1.5 |
| AA874101_tumor-associated_calcium_signal_transducer_2 | −1.5 | −1.5 | −2.4 | −1.2 |
| AA474336_synaptonemal_complex_protein_3 | −2.1 | −1.4 | −2.3 | −1.3 |
| AA031015_myloperoxidase | −1.3 | −1.3 | 1.0 | 1.1 |
| AA189266_ESTs | −3.0 | −1.8 | −3.9 | −1.4 |
| AA060282_neutrophilic_granule_protein | 1.2 | −1.1 | 1.0 | −1.4 |

| | | | | | 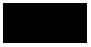 | | 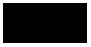 | |
|---|---|---|---|---|---|---|---|---|
| | | | | | (−) | (+) | (−) | (+) |
| IDI | Description | No. Tiss | 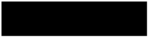 | 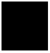 | PL-4B_BDE | PL-16B_BDE | PL-2B_BDE | PL-14B_BDE |
| 168 | AA756136_actin_,_gamma_2,_smooth_muscle_enteric | 1 | intestine | blad | −3.5 | −3.7 | −8.8 | −7.2 |
| 166 | W54383_glutathione_S-transferase,_mu_2 | 1 | intestine | blad | −5.2 | −7 | −1.4 | −2.5 |
| 161 | 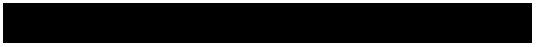 | 1 | intestine | blad | −1.3 | −1.4 | −2.9 | −3.1 |
| 44 | AA445731_heat_shock_protein_cognate_70,_testis | 2 | spleen; lung | blad | 1.1 | −1.7 | −2.8 | −2.9 |
| 286 | W53916_RIKEN_cDNA_3300001P10_gene | 1 | spleen | blad | −3.3 | −6.3 | −2.4 | −3.6 |
| 47 | 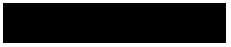 | 2 | stomach; bladder | blad | [symbol] | −1.3+ | 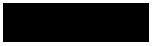 | 1.1 |
| 252 | 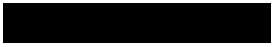 | 1 | mammary gland | blad, breast | −2.1 | −2 | 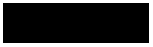 | 1.5 |
| 98 | 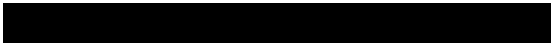 | 1 | brain | brain | −1.1 | −1.4 | 1.5 | 1.3 |
| 94 | 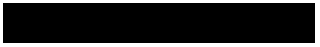 | 1 | brain | brain | [symbol] | 1.5 | −1 | −1 |
| 104 | 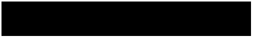 | 1 | brain | brain | [symbol] | −1.7+ | 1.1+ | −1.9 |
| 122 | 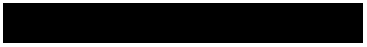 | 1 | colon | brain | −1.6 | −1 | −1.1 | −1.1 |
| 115 | 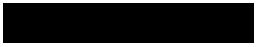 | 1 | colon | brain | −2.3 | 1 | 1.5 | 1.4 |
| 118 | 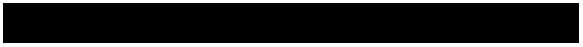 | 1 | colon | brain | −1.2 | 1.2 | 1.3 | 1.5 |
| 116 | 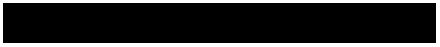 | 1 | colon | brain | −1.2 | 1.3 | 1.3 | 1.4 |
| 128 | 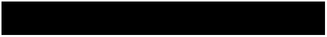 | 1 | colon | brain | −1 | 1.3 | 1.1 | −1.1 |
| 189 | 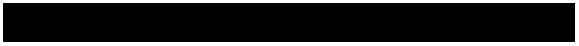 | 1 | kidney | brain | −2.5 | −2.8 | −1.1 | −1 |
| 235 | 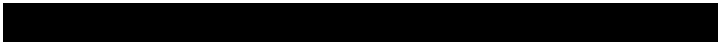 | 1 | liver | brain | −1.2 | −4.3 | −2.3 | −2.5 |
| 269 | 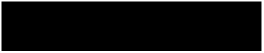 | 1 | pancreas | brain | −4.2 | −3.4 | −1.3 | −3 |
| 262 | 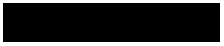 | 1 | pancreas | brain | −3.8 | −2.7 | −1.2 | −2.7 |
| 263 | 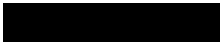 | 1 | pancreas | brain | −2.7 | −2.5 | −1 | −2.3 |
| 278 | AA107928_reduced_expression_3 | 1 | placenta/uterus | brain | −1.9 | −2. | −1.3 | −1.5 |
| 283 | AI510089_ESTs | 1 | spleen | brain | [symbol] | −5.9* | [symbol] | −2.4 |
| 50 | 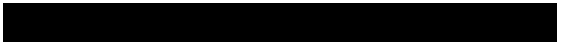 | 2 | stomach; brain | brain | −1.2 | −1.4 | 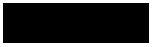 | 1.5 |
| 58 | 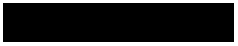 | 2 | stomach; colon | brain | −2.2 | −1.2 | 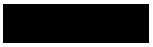 | 1.5 |
| 337 | 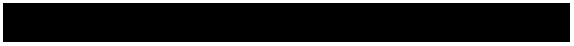 | 1 | stomach | brain | −1.9 | −2.5 | 1.1 | 1 |
| 333 | 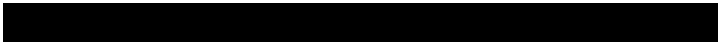 | 1 | stomach | brain | −1.7 | −2.1 | 1.3 | 1.1 |
| 357 | 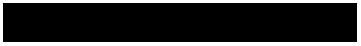 | 1 | stomach | brain | −1.3 | −1.4 | 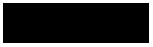 | 1.6 |
| 309 | 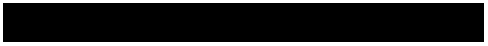 | 1 | stomach | brain | −1.7 | −1.5 | 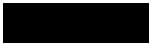 | 1.6 |
| 305 | 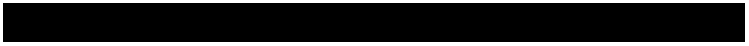 | 1 | stomach | brain, blad | −1.6 | −1.7 | 1.5 | 1.4 |
| 155 | 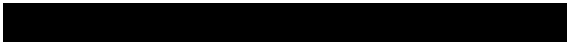 | 1 | intestine | brain, blad | −1.3 | −1.2 | −2.6 | −3.4 |
| 292 | 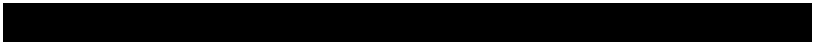 | 1 | stomach | brain, pl/ut | 1 | [symbol] | 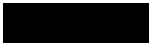 | 1.6 |
| 120 | 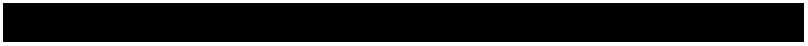 | 1 | colon | breast | −1.1 | −1.1 | [symbol] | −4.2 |
| 202 | 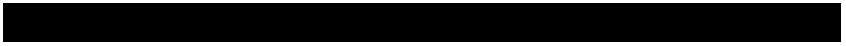 | 1 | liver | breast | −1.0 | −1.2 | −1.1 | −7.2 |
| 196 | 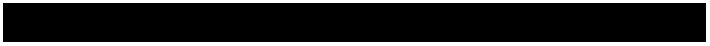 | 1 | liver | breast | −1.7 | −1.7 | −1.6 | −1.2 |
| 253 | 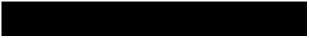 | 1 | mammary gland | breast | −3.4 | −5.3 | −2.5 | −2 |
| 250 | 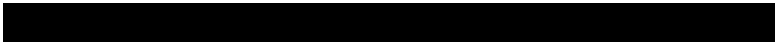 | 1 | mammary gland | breast | −3.7 | −3.5 | −2.3 | −1.3 |
| 277 | AA754922_stearyl-Coenzyme_A_desaturase_1 | 1 | placenta/uterus | breast | −4.9 | −4.4 | −3.2 | [symbol] |
| 290 | AA792297_procollagen,_type_V,_alpha_1 | 1 | stomach | breast | −1.3 | −1.1 | −1.1 | [symbol] |
| 197 | AA474336_synaptonemal_complex_protein_3 | 1 | liver | col | −3 | −1.1 | [symbol] | 1.3 |
| 154 | AI594147_beta-2_microhlobulin | 1 | intestine | col, breast | 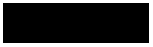 | 1.2 | −4.4 | −1.6 |
| 164 | AA109873_RIKEN_cDNA_0610010E05_gene | 1 | intestine | col, breast | −2.8 | −2.6 | −3.4 | −3.1 |
| 401 | AA068624_hydroxyacid_oxidase_(glycolate_oxidase)_3 | 1 | stomach | col, kidn | −1.2 | [symbol] | 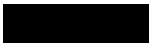 | 1.6 |
| 151 | AI323162_dipeptidase_I_(renal) | 1 | intestine | col, lung, kidn | [symbol] | −3.8 | −1.8 | −1.4 |
| 152 | 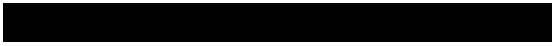 | 1 | intestine | col, lung, kidn | −1.6 | −2.4 | [symbol] | −1.6 |
| 156 | AA793433_glutamyl_aminopeptidase | 1 | intestine | col, pl/ut | −2.2 | −2.9 | −1.4 | −1.5 |
| 181 | 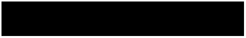 | 1 | intestine | col, pl/ut | −1.2 | −1.2 | −2.1 | −2 |
| 107 | 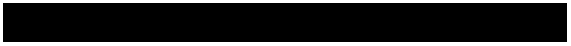 | 1 | colon | int | −3.7 | −1.8 | −1.7 | 1.3 |
| 129 | 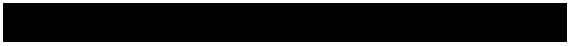 | 1 | colon | int | −2.3 | −1.7 | −1 | 1.1 |
| 160 | 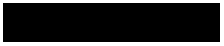 | 1 | intestine | int | −1.3 | −1 | −1.5 | −1.2 |
| 17 | 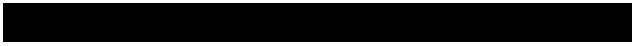 | 2 | colon; mammary gland | int, col | −7.2 | −4.9 | −4.3 | −2.8 |
| 19 | 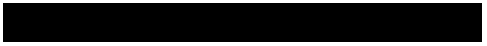 | 2 | colon; placenta/uterus | int, col | −4.4 | −3.7 | −1.4 | −1.6 |
| 136 | 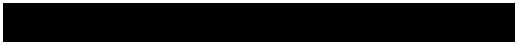 | 1 | colon | int, col | −1.5 | −7.9 | −6.6 | −5.1 |
| 112 | 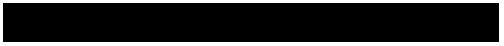 | 1 | colon | int, col | −2.5 | −2.4 | 1 | −1.1 |
| 25 | 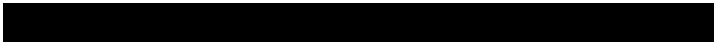 | 2 | intestine; colon | int, col | −3.9 | −3.9 | −1.4 | −1.8 |
| 26 | 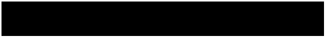 | 2 | intestine; colon | int, col | −3.7 | [symbol] | −1.3 | −1.3 |
| 27 | 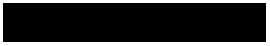 | 2 | intestine; colon | int, col | −2.4 | −2.3 | −1.5 | −1.2 |
| 163 | 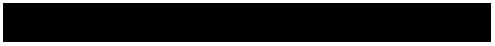 | 1 | intestine | int, col | −1.3 | −8.6 | −5.2 | −4.6 |
| 177 | 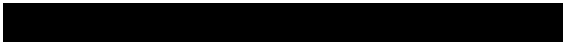 | 1 | intestine | int, col | −1.7 | −2.3 | −2 | −1.5 |
| 167 | 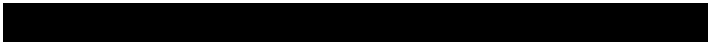 | 1 | intestine | int, col | −1.9 | −1.9 | −1.4 | −1.4 |
| 178 | 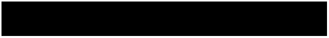 | 1 | intestine | int, col | −1.5 | −1.5 | 1.4 | 1.4 |
| 174 | 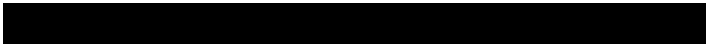 | 1 | intestine | int, col | −7.5 | [symbol] | −5.3 | −3.6 |
| 183 | AA481911_Mus_musculus,_clone_MGC:6227,_mRNA_com- | 1 | kidney | int, col | −1.1 | 1 | −13 | −6.5 |
| | plete_cds | | | | | | | |
| 226 | AA237607_pyruvate_kinase_liver_and_red_blood_cell | 1 | liver | int, col | −1.2 | 1.2 | −2.8 | −2.4 |
| 257 | AA242102_villin | 1 | mammary gland | int, col | −4.9 | −5.2 | −3.4 | −2.4 |
| 38 | 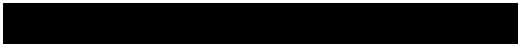 | 2 | pancreas; intestine | int, col | −8.9 | −15 | 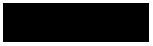 | −2.6 |
| 60 | 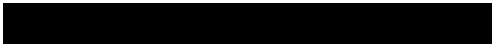 | 2 | stomach; colon | int, col | −1.5 | −7.9 | −1.0 | [symbol] |
| 62 | 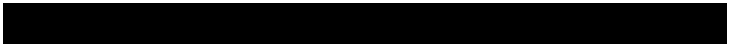 | 2 | stomach; intestine | int, col | [symbol] | −7.8 | −3.6 | −3.4 |
| 64 | AA146478_M_musculus_mRNA_)3C10)_for_IgA_V- | 2 | stomach; kidney | int, col | −1.9 | −1.4 | −2.8 | −2.9 |
| | D-J-heavy_chain | | | | | | | |
| 12 | AA445667_RIKEN_cDNA_4632401C08_gene | 3 | stomach; bladder; mammary gland | int, col, kidn | −2.3 | −1 | −1.1 | 1.1 |
| 148 | AA023491_RIKEN_cDNA_1600012D06_gene | 1 | colon | int, col, pl/ut | −1.2 | −1.5 | 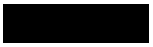 | 1.5 |
| 106 | 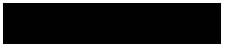 | 1 | brain | int, kidn | −2.4 | −2.5 | −1.5 | −1.3 |
| 280 | AI614443_3′-phosphoadenosine_5′- | 1 | placenta/uterus | int, kidn | −2.7 | −3.2 | −1.8 | −1.4 |
| | phosphosulfate_synthase_2 | | | | | | | |
| 24 | AA656694_cytochrome_P450,_2b9,_phenoberbitol_in- | 2 | intestine; colon | int, lung | −3.2 | −3 | −1.5 | −1.1 |
| | ducible,_type_a | | | | | | | |
| 32 | AA325330_cytochrome_P450,_2b13,_phenoberbitol_in- | 2 | liver; intestine | int, lung | −2.3 | −2.3 | −2.2 | −2 |
| | ducible,_type_c | | | | | | | |
| 8 | AA107035_guanylate_cyclase_activator_2b_(retina) | 3 | intestine; colon; mammary gland | int, pl/ut | −22 | −19 | −21 | −16 |
| 258 | W17866_urine-specific_proline-rich_acid_protein | 1 | mammary gland | int, pl/ut | −5.8 | −4.3 | −8.2 | −4.9 |
| 149 | AA108495_deocyribonuclease_1 | 1 | colon | kidn | −14 | 15 | −1.4 | −7.4 |
| 117 | AA822473_DNA_seg- | 1 | colon | kidn | −7.8 | −7.6 | −5.2 | −3.3 |
| | ment,_Chr_8,_brighman_&_Women's_Genetics_1320_expressed | | | | | | | |
| 132 | AI181090_cytochrome_P450_subfamily_IV_B,_polypeptide_I | 1 | colon | kidn | −4.8 | −5 | −8.3 | −5.7 |
| 141 | AI386288_RIKEN_cDNA_2700043D08_gene | 1 | colon | kidn | 1.1 | 1.1 | −1.4 | −1.2 |
| 170 | 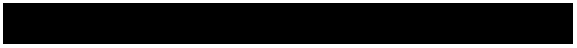 | 1 | intestine | kidn | −1.4 | −1.3 | −1.1 | −1.2 |
| 180 | 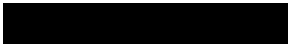 | 1 | intestine | kidn | −1.2 | −1.1 | −1.4 | −1.5 |
| 29 | 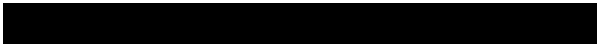 | 2 | kidney; mammary gland | kidn | −2.1 | −3.4 | −1.2 | −1.2 |
| 187 | 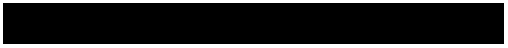 | 1 | liver | kidn | −1 | [symbol] | [symbol] | 1.2 |
| 219 | 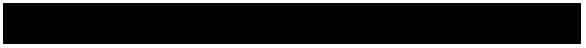 | 1 | liver | kidn | −1.1 | −1.3 | −2.2 | −1.8 |
| 222 | 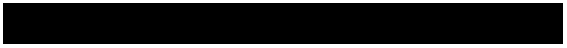 | 1 | liver | −1.8 | −2.4 | −1.3 | −1.5 | |
| 431 | AA617362_low_density_lipoprotein_receptor- | 1 | stomach | kidn | −1.9 | −2.2 | 1.2 | −1.2 |
| | related_protein_2 | | | | | | | |
| 439 | AI587821_solute_carrier_family_22_(organic_cation_trans- | 1 | stomach | kidn | −1.8 | −2.5 | −1 | −1.2 |
| | porter),_member_2 | | | | | | | |
| 321 | AA110551_phospholipid_scramblase_2 | 1 | stomach | kidn | −2.4 | −2.7 | −1.4 | −1.2 |
| 165 | AA415344_histone_deacetylase_7 | 1 | intestine | kidn. pl/ut | −1.1 | −1.3 | −1.2 | −2 |
| 254 | W14224_N-myc_downstream_regulated_I | 1 | mammary gland | kidn, pl/ut | −3.1 | −3.3 | −2.1 | −2.2 |
| 30 | 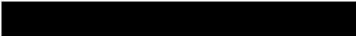 | 2 | liver, colon | liv | 1.3 | [symbol] | −1.8 | −1.2 |
| 213 | 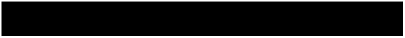 | 1 | liver | liv | −1.4 | −2 | −1.5 | −1.5 |
| 207 | 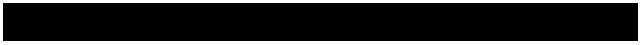 | 1 | liver | liv | −3.2 | −4.1 | [symbol] | −2.3 |
| 205 | 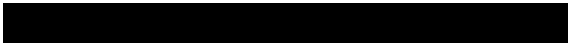 | 1 | liver | liv | [symbol] | −1.4 | [symbol] | [symbol] |
| 237 | 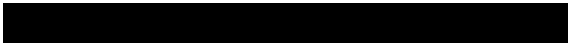 | 1 | liver | liv | 1.3 | 1.5 | 1+ | 1.1 |
| 211 | 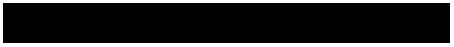 | 1 | liver | liv | −1.1 | −1.2 | −2.1 | −2 |
| 233 | 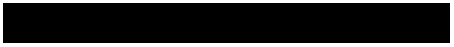 | 1 | liver | liv | −2.9 | −3 | −1.7 | −1.5 |
| 230 | 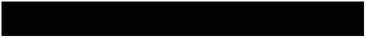 | 1 | liver | liv | −1.5 | −2.5 | −1.5 | −1.2 |
| 42 | AI386058_serum_albumin_variant | 2 | spleen; liver | liv | −1.8 | −5.3 | −29 | −1.7 |
| 124 | AA067003_glutathione_S-transferase,_mu_I | 1 | colon | liv, blad | −1.5 | [symbol] | −2.4 | −2.9 |
| 214 | AA261489_hypothetical_protein,_154 | 1 | liver | liv, brain, kidn | −2.5 | [symbol] | −1.8 | [symbol] |
| 111 | AA230638_transcription_factor-like_protein_ODA-10 | 1 | colon | liv, breast | −1.1 | −1.1 | 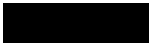 | 11 |
| 195 | AI386062_carbonic_anhydrase_3 | 1 | liver | liv, breast | −11 | −13 | −8.3 | −6.3 |
| 209 | 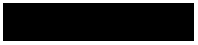 | 1 | liver | liv, col | 1.4 | 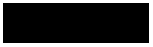 | 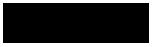 | 1.4 |
| 215 | 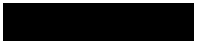 | 1 | liver | liv, col | 1.2 | 1.5 | 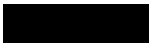 | 1.4 |
| 22 | 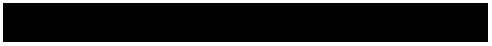 | 2 | intestine; colon | liv, colon, kidn | −2 | −2.3 | 1.1 | −1.3 |
| 220 | 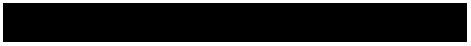 | 1 | liver | liv, col, lung, kidn | −3.1 | −3.3 | −4 | −2.8 |
| 171 | AA647336_cytochrome_P450,_steroid_inducible_3a11 | 1 | intestine | liv, int | −4.6 | −6.9 | −3.8 | −2.6 |
| 175 | AA237986_cytochrome_P450,_3a25 | 1 | intestine | liv, int | −2.4 | −3 | −1.1 | 1.1 |
| 186 | 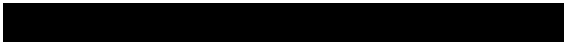 | 1 | kidney | liv, int | −1.4 | −1.7 | 1.2 | 1 |
| 206 | AA822117_UDP- | 1 | liver | liv, int, blad, | −5.1 | −1.9 | −4.8 | −3.5 |
| | glucuronosyltransferase_1_family,_member_1 | | | kidn | | | | |
| 21 | 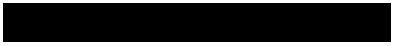 | 2 | intestine; colon | liv, int, col | −32 | −11 | −13 | −8.6 |
| 143 | AA510906_Mus_musculus_Cyp4v_mRNA_for_family_4_cyto- | 1 | colon | liv, int, col | −2 | −2 | −1.8 | −1.2 |
| | chrome_P450,_complete_cds | | | | | | | |
| 249 | AA290390_cell_death- | 1 | mammary gland | liv, int, col, | −4.2 | −4.9 | −4.1 | −2.7 |
| | inducing_DNA_fragmentation_factor,_alpha_subunit- | | | kidn | | | | |
| | like_effector_B | | | | | | | |
| 134 | AA895199_ketohexokinase | 1 | colon | liv, int, kidn | [symbol] | [symbol] | −2.5 | −2.3 |
| 241 | AA237317_solute_carrier_family_2_(facilitated_glucose_trans- | 1 | liver | liv, int, kidn | −1.7+ | −1.9+ | −1.4+ | −1.7+ |
| | porter),_member_2 | | | | | | | |
| 198 | AI507919_alcohol_dehydrogenase_1,_complex | 1 | liver | liv, int, kidn | −3.4 | −3.9 | −1.7 | −1.4 |
| 184 | AA108417_insulin-like_growth_factor_binding_protein_4 | 1 | kidney | liv, kidn | −2.1 | −2 | −6.1 | −4.1 |
| 185 | AA145454_insulin-like_growth_factor_binding_protein_4 | 1 | kidney | liv, kidn | −1.9 | −1.9 | −4.9 | −3.2 |
| 193 | 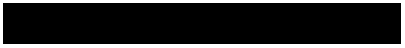 | 1 | kidney | liv, kidn | −3.4 | −4.1 | −2.1 | −1.6 |
| 33 | 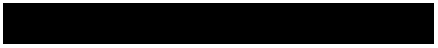 | 2 | liver; kidney | liv, kidn | −5.6 | −6.6 | −3.2 | −1.9 |
| 34 | 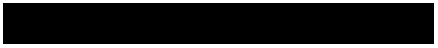 | 2 | liver; kidney | liv, kidn | −5.8 | −7.7 | −4.3 | −3.4 |
| 229 | AA245848_hydroxysteroid_dehydrogenase-3,_delta<5>-3-beta | 1 | liver | liv, kidn | −3.3 | −5.4 | −3.6 | −2.8 |
| 430 | AA096985_cytochrome_P450,_2j5 | 1 | stomach | liv, kidn | −1.5 | [symbol] | [symbol] | 1.5 |
| 227 | 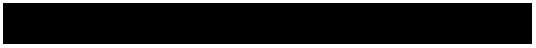 | 1 | liver | liv, lung | −1.9 | −1.9 | −2.3 | −2.1 |
| 199 | 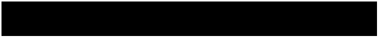 | 1 | liver | liv, lung, kidn | −6.2 | −7.7 | −3.2 | −2.4 |
| 212 | 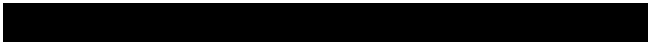 | 1 | liver | liv, lung, pl/ut | −1.1 | −1.9 | −2.2 | −2.3 |
| 228 | AA027607_hydroxysteroid_17-beta_dehydrogenase_2 | 1 | liver | liv, stom, col, | −1.3+ | −1.6+ | −1.2+ | 1.1 |
| | | | | blad, pl/ut | | | | |
| 217 | AA108741_RIKEN_cDNA_0610025119_gene | 1 | liver | liv, stom, int | −2 | −2.2 | −1.1 | −1.1 |
| 139 | AA982227_glutathione_S-transferase,_alpha_3 | 1 | colon | liv, stom, lung | −2.8 | −3.7 | −1.6 | −1.5 |
| 223 | AI597312_hydroxysteroid_dehydrogenase-1_delta<5>-3-beta | 1 | liver | liv, kidn | −2.9 | −3.6 | −2 | −1.3 |
| 80 | 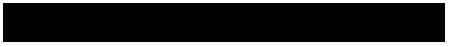 | 1 | bladder | lung | −2 | −1.9 | −1.5 | −1.1 |
| 147 | AA982240_advanced_glycosylation_end_product- | 1 | colon | lung | −1.4 | −1.4 | −1.2 | −1 |
| | specific_receptor | | | | | | | |

| | | 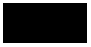 | | 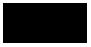 | | 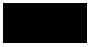 | | 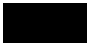 | |
|---|---|---|---|---|---|---|---|---|---|
| | | (−) | (+) | (−) | (+) | (−) | (+) | (−) | (+) |
| IDI | Description | PL-6B_BDE | PL-18B_BDE | PL-8B_BDE | PL-20B_BDE | PL-3B_BDE | PL-15B_BDE | PL-5B_BDE | PL-17B_BDE |
| 168 | AA756136_actin_,_gamma_2,_smooth_muscle_enteric | −5.9 | −6.3 | 1.4 | 1.4 | −1.1 | −2.2 | 1.1 | 1.3 |
| 166 | W54383_glutathione_S-transferase,_mu_2 | −1 | 1 | 1.1 | 1.6 | 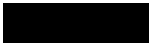 | −1.1 | −1.7 | −2 |
| 161 | 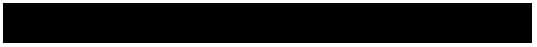 | −1.4 | −2.1 | −1.6 | −1.9 | 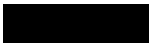 | 1.2 | 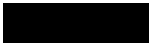 | 1.1 |
| 44 | AA445731_heat_shock_protein_cognate_70,_testis | −1.2 | −1.2 | 1.1 | 1.4 | −1.7 | −1.5 | −1.2 | 1.1 |
| 286 | W53916_RIKEN_cDNA_3300001P10_gene | −3.5 | −3.5 | 1.6 | 1.4 | −3 | −3.8 | −4.7 | −2.8 |
| 47 | 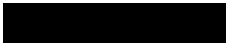 | 1.1 | 1.3+ | 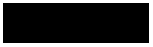 | −1.1 | 1.5 | −1.2 | 1.2 | [symbol] |
| 252 | 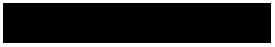 | 1.2 | −1.1 | −1.2 | −1.3 | −1.2 | −1.3 | 1.2 | 1.2* |
| 98 | 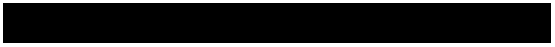 | −1.3 | −1.5 | −1 | −1.3 | −1.4 | −1.5 | [symbol] | −1.1+ |
| 94 | 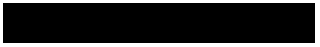 | −1.3 | −1.2 | −1.9 | −1.6 | −1.7 | −1.1 | −1.3 | −1.3 |
| 104 | 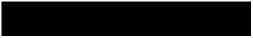 | −1.6+ | −2 | −1.6 | [symbol] | −1.4+ | −2.4+ | −1.7 | −2.5+ |
| 122 | 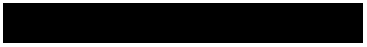 | −1.4 | −1 | −1 | −1.1 | −1.1 | −1.1 | 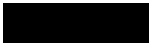 | −1.3 |
| 115 | 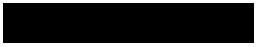 | 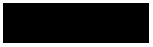 | 1.4 | 1.1 | 1.2 | 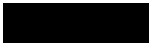 | 1.4 | 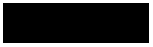 | 1.2* |
| 118 | 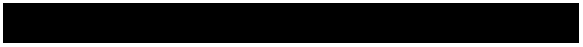 | 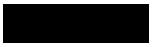 | 1.2 | 1.2 | 1.1 | 1.3 | 1.1 | 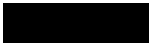 | 1.3* |
| 116 | 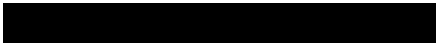 | 1.6 | 1.4 | 1.1 | −1.1 | 1.3 | 1.2 | 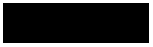 | −1 |
| 128 | 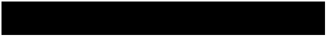 | 1.6 | 1.1 | −1 | 1.1 | 1.2 | −1 | 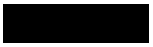 | 1.1 |
| 189 | 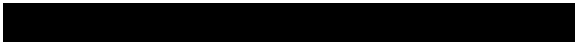 | −1.1 | −1.3 | −1.3 | −1.1 | −2 | −2.5 | −2.3 | −1.8 |
| 235 | 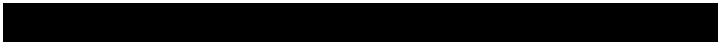 | 1.4 | −1.4 | −1.4 | −1.3 | 1.4 | −1.1 | −1.5 | −1.5 |
| 269 | 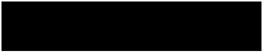 | −3.9 | −4.9 | −1.9 | −2 | −3.5 | −5 | −2.5 | −2 |
| 262 | 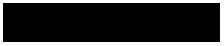 | [symbol] | −3.5 | −1.6 | −1.2 | −2.9 | −3.6 | −2.2 | 1.1 |
| 263 | 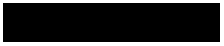 | 3.3 | −3 | −1.4 | −1.5 | −2.8 | −3.2 | −2.5 | −1.6* |
| 278 | AA107928_reduced_expression_3 | −1.1 | −1.6 | −1.2 | −1.3 | −1.6 | −1.7 | 1.1 | −1.6 |
| 283 | AI510089_ESTs | −2.5 | [symbol] | −2.7 | −1.1 | [symbol] | −3.8 | −2.7 | −2.7 |
| 50 | 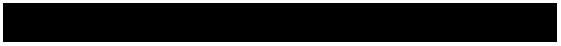 | 1.1 | 1 | 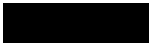 | −1.2 | 1.3 | −1.1 | −1.1 | −1* |
| 58 | 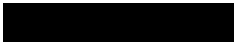 | 1.4 | 1.2 | 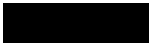 | −1.3 | 1.5 | −1.1 | 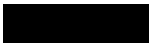 | −1.3* |
| 337 | 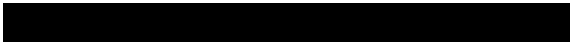 | −1.7 | −1.1 | 1.3 | −1.6 | −1.1 | −1.7 | −1.5 | −2.6 |
| 333 | 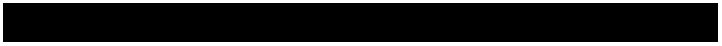 | −1.4 | −1.2 | 1.2 | −1.8 | −1.2 | −1.5 | −1.3 | −1.5+ |
| 357 | 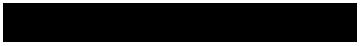 | −1 | 1.5 | 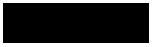 | −1.2 | 1.4 | 1 | 1.1 | −1.3+ |
| 309 | 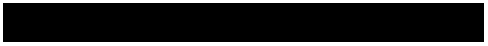 | 1.4 | 1 | 1.4 | −1.5 | 1.1 | −1.4 | 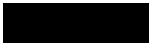 | −1 |
| 305 | 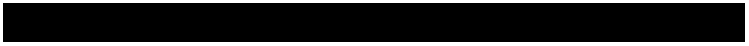 | −1.3 | −1.1* | 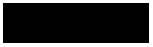 | −1.3 | 1.2 | −1.3 | −1.3 | −1.5 |
| 155 | 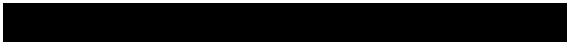 | −1.3 | −1.4 | −4.1 | −2.9 | 1.2 | −2.3 | −1.5 | −2.1 |
| 292 | 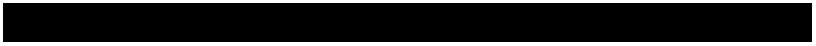 | 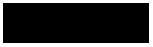 | 1.6 | 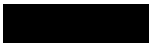 | −1.1 | 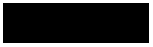 | 1.4 | 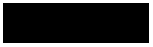 | [symbol] |
| 120 | 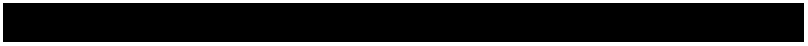 | −1.9 | [symbol] | −4.7 | −5.6 | 6 | −7.1 | −3.7 | −7.6 |
| 202 | 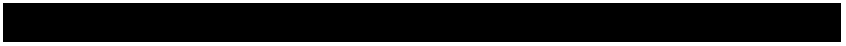 | 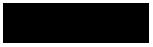 | −1 | −6.9 | −5 | −13 | −11 | −9.3 | −5.8 |
| 196 | 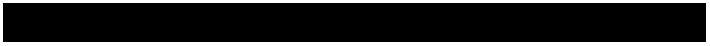 | 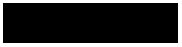 | 1.4 | −1.1 | −1.1 | −1.6 | −1.6 | −1.9 | [symbol] |
| 253 | 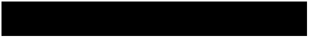 | −3.7 | −2.4 | −2.9 | −2.4 | −3.4 | −3.1 | −2.9 | −2.4 |
| 250 | 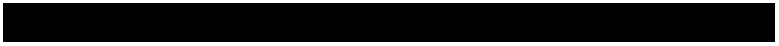 | −16 | −19 | '116 | −20 | −24 | −22 | −12 | −15 |
| 277 | AA754922_stearyl-Coenzyme_A_desaturase_1 | −3.5 | −2.5 | −1.9 | −3.4 | [symbol] | −4.7 | −3.5 | −5.6* |
| 290 | AA792297_procollagen,_type_V,_alpha_1 | −1.4* | −1.2 | 1.3 | −1.5 | −1.1 | −1.6 | −1.2 | −1.7 |
| 197 | AA474336_synaptonemal_complex_protein_3 | 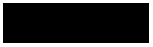 | 1.2 | 1.4 | 1 | 1.5 | 1.1 | 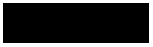 | 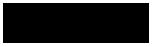 |
| 154 | AI594147_beta-2_microhlobulin | 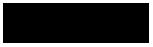 | [symbol] | −1.6 | 1.3* | 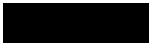 | 1.4 | 1.4 | 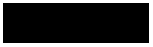 |
| 164 | AA109873_RIKEN_cDNA_0610010E05_gene | −2.8 | −3.1 | −3.3 | −3.2 | 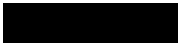 | 1.2 | 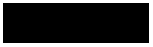 | 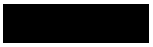 |
| 401 | AA068624_hydroxyacid_oxidase_(glycolate_oxidase)_3 | 1 | 1.3 | 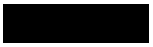 | −1.2 | 1.4 | 1.1* | 1.6 | 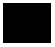 |
| 151 | AI323162_dipeptidase_I_(renal) | −2.9 | −2 | −1.8 | −2.3 | 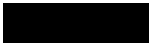 | 1.2 | 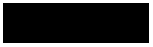 | 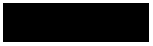 |
| 152 | 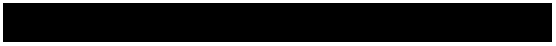 | −1.7 | −2.3 | −1.5 | −1.6 | 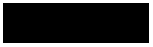 | 1.1 | 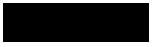 | 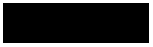 |
| 156 | AA793433_glutamyl_aminopeptidase | −1.6 | −1.7 | −1.2 | −1.6 | 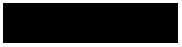 | 1.6 | 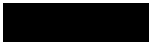 | 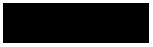 |
| 181 | 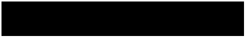 | −1.1 | 1 | −2.1 | −2 | 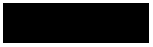 | 1.3 | 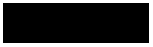 | 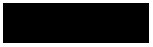 |
| 107 | 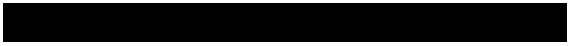 | −1.7 | −1 | −1.2 | 1.6 | 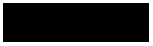 | 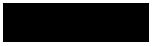 | 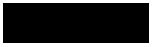 | 1.6 |
| 129 | 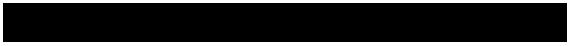 | −1.6 | 1.1 | −1 | 1.5 | 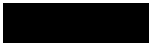 | 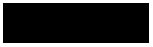 | 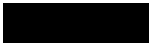 | 1.5 |
| 160 | 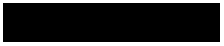 | −1.6 | −1.7 | −1.4 | −1.1 | 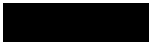 | 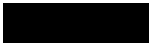 | 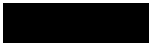 | 1.1 |
| 17 | 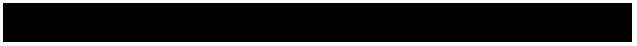 | −3 | −1.7 | −2 | −3.5 | 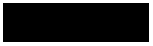 | 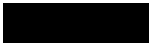 | 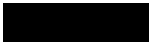 | 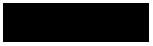 |
| 19 | 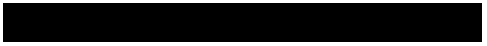 | −3.1 | −2.6 | −2 | −3 | 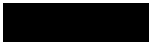 | 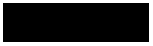 | 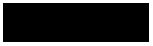 | 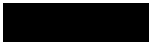 |
| 136 | 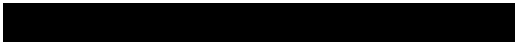 | −6.9 | −6.1 | −5.8 | −9.1 | 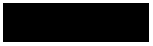 | 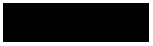 | 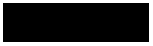 | 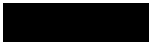 |
| 112 | 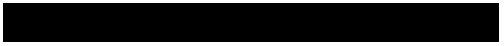 | −2.3 | −2.1 | −2.1 | −2.3 | 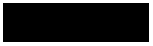 | 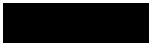 | 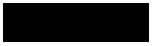 | 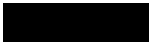 |
| 25 | 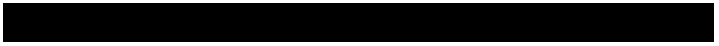 | −2.8 | −2 | −1.5 | −2.8 | 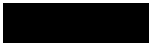 | 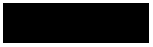 | 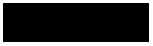 | 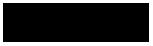 |
| 26 | 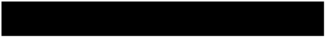 | −1.7 | −1.5 | −1.1 | −1.9 | 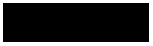 | 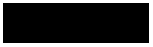 | 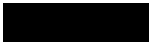 | 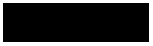 |
| 27 | 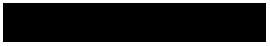 | −1.2 | −1.1 | −1 | −1.7 | 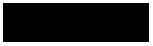 | 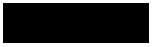 | 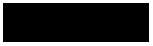 | 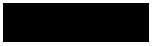 |
| 163 | 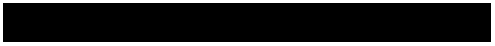 | −5.5 | −5.3 | −5.1 | −8.9 | 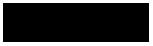 | 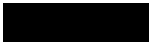 | 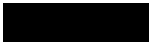 | 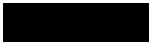 |
| 177 | 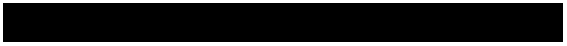 | −1.2 | −1.3 | −1.3 | −1.1 | 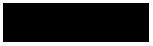 | 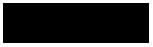 | 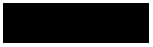 | 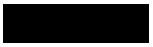 |
| 167 | 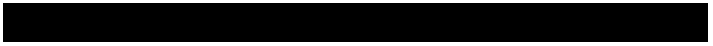 | −1.2 | −1.2 | 1 | −1.3 | 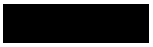 | 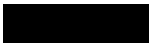 | 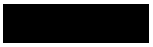 | 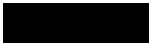 |
| 178 | 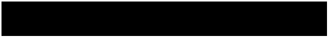 | −1.1 | 1.1 | 1.2 | −1 | 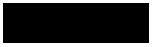 | 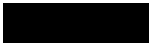 | 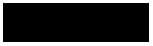 | 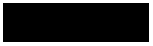 |
| 174 | 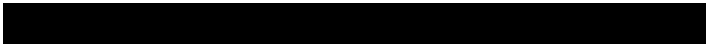 | −5.3 | −3.4 | −3.3 | −5.5 | 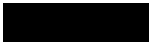 | −1.9 | 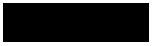 | 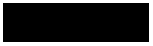 |
| 183 | AA481911_Mus_musculus,_clone_MGC:6227,_mRNA_com- plete_cds | −1.4 | −7.5 | −13 | −13 | 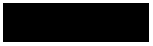 | 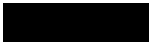 | 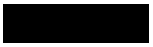 | 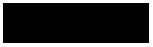 |
| 226 | AA237607_pyruvate_kinase_liver_and_red_blood_cell | 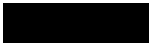 | 1.6 | −1.5 | −1.2 | 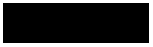 | 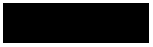 | 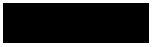 | 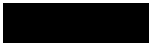 |
| 257 | AA242102_villin | −3.6 | −2.5 | −1.4 | −1.8 | 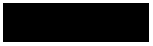 | 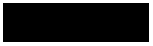 | 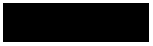 | 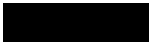 |
| 38 | 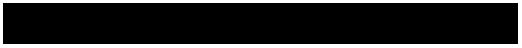 | −9.9 | −5.2 | −4.8 | −5.8 | 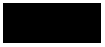 | 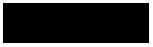 | 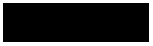 | 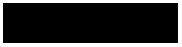 |
| 60 | 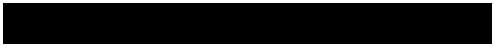 | −9.1 | −6.3 | −4.9 | −9.6 | 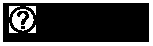 | 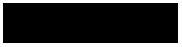 | 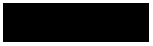 | 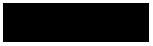 |
| 62 | 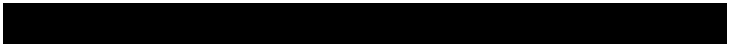 | −5 | −3 | −3 | −5.9 | 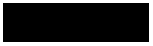 | 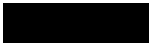 | 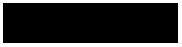 | 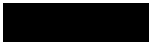 |
| 64 | AA146478_M_musculus_mRNA_)3C10)_for_IgA_V- D-J-heavy_chain | −1.7 | −3 | −1.8 | −4.9 | 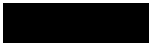 | 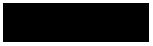 | 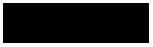 | 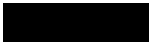 |
| 12 | AA445667_RIKEN_cDNA_4632401C08_gene | −2.4 | −1.2 | 1.3 | −1.7 | 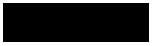 | 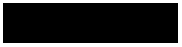 | 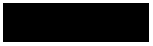 | 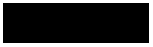 |
| 148 | AA023491_RIKEN_cDNA_1600012D06_gene | −1.1 | −1.1 | 1.2 | −1.1 | 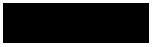 | 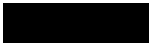 | 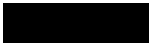 | 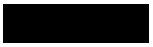 |
| 106 | 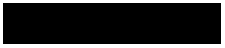 | 1+ | 1 | −1.5 | −1.8 | 1.1 | 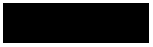 | 1.3 | 1.5* |
| 280 | AI614443_3′-phosphoadenosine_5′- phosphosulfate_synthase_2 | 1.1 | −1.3 | 1.3 | 1.1 | 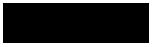 | 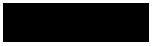 | −1.4 | −1 |
| 24 | AA656694_cytochrome_P450,_2b9,_phenoberbitol_in- ducible,_type_a | 1.4 | −1.3 | −1.3 | −1.8 | 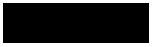 | 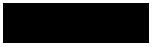 | 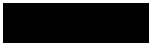 | −2 |
| 32 | AA325330_cytochrome_P450,_2b13,_phenoberbitol_in- ducible,_type_c | −1.1 | −2.1 | −1.7 | −1.7 | 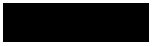 | 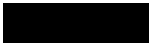 | 1.3 | −2.1+ |
| 8 | AA107035_guanylate_cyclase_activator_2b_(retina) | −15 | −16 | −17 | −16 | 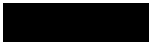 | 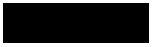 | 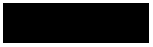 | −1.2 |
| 258 | W17866_urine-specific_proline-rich_acid_protein | −5 | −4.2 | −7.1 | −5.1 | 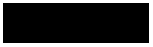 | 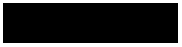 | 1.3**** | −1.4 |
| 149 | AA108495_deocyribonuclease_1 | −8 | −8.3 | −7.1 | −8.5 | −2.1 | −3 | −3.6 | −7.7 |
| 117 | AA822473_DNA_seg- | 1.1 | −1.2 | −3.3 | −3.2 | −4.8 | −4.5 | −2.6 | −7.6 |
| | ment,_Chr_8,_brighman_&_Women's_Genetics_1320_expressed | | | | | | | | |
| 132 | AI181090_cytochrome_P450_subfamily_IV_B,_polypeptide_I | −5.4 | −4.9 | −7.4 | −6.5 | −2 | −2.2 | −2.7 | −5.1 |
| 141 | AI386288_RIKEN_cDNA_2700043D08_gene | 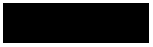 | 1.3 | −1.4 | −1.8 | −1.5 | −1.3 | −1.1 | −2.2 |
| 170 | 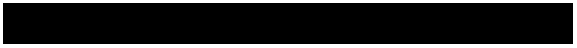 | −1.4 | −1.2 | −1.2 | −1.1 | 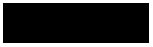 | 1.3 | 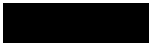 | 1.5 |
| 180 | 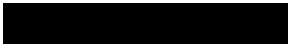 | −1.2 | −1.4 | −1.3 | −1.6 | 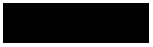 | 1.3 | 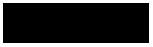 | 1.5+ |
| 29 | 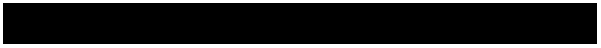 | −2.5 | −1.4 | −1 | −2 | [symbol] | −2.7 | −2.3 | −2.5 |
| 187 | 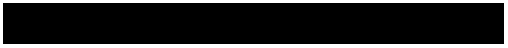 | 1.2 | −1 | −1.2 | −1.1 | −1.1 | −1 | 1.2 | 1.2 |
| 219 | 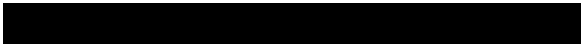 | 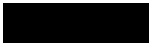 | 1.1 | −1.6 | −1.7 | 1.2 | 1 | 1.1 | 1.1 |
| 222 | 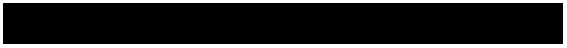 | 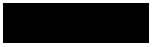 | 1.3 | −1.5 | −1.4 | −1.1 | −1 | −1.4 | −1.4 |
| 431 | AA617362_low_density_lipoprotein_receptor- | −1.3 | −1.1 | 1.2 | [symbol] | −1.2 | [symbol] | −1.2 | [symbol] |
| | related_protein_2 | | | | | | | | |
| 439 | AI587821_solute_carrier_family_22_(organic_cation_trans- | −1.8 | −1.4 | −1.2 | −1.7 | −1.2 | −1.8 | −1.6 | −1.5 |
| | porter),_member_2 | | | | | | | | |
| 321 | AA110551_phospholipid_scramblase_2 | −1.5 | −1.4 | 1.1 | −1.9 | 1.2 | −1.4 | 1.3 | 1.5 |
| 165 | AA415344_histone_deacetylase_7 | −1.6 | −3 | −1.3 | −4.1 | 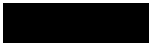 | 1.1 | 1.2 | −1.1 |
| 254 | W14224_N-myc_downstream_regulated_I | −4.7 | −4.8 | −1.1 | −1.2 | −1.1 | −2 | −1.2 | −1.2 |
| 30 | 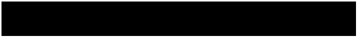 | 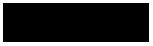 | 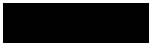 | −1.6 | −2 | −1.7 | −1.3 | 1.1 | −1.7 |
| 213 | 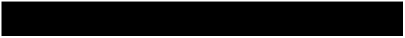 | 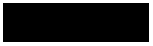 | 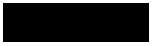 | −1.2 | −1 | −1.6 | −2 | −1.7 | −1.9 |
| 207 | 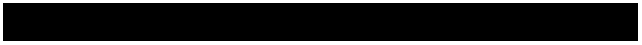 | 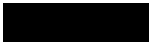 | 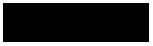 | −2 | −2 | −2.8 | −3 | −2.9 | −1.8 |
| 205 | 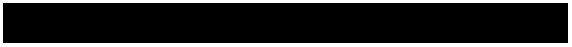 | 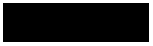 | 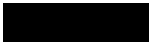 | −1.1 | 1.1 | 1 | −1.1 | 1.1 | −1.1+ |
| 237 | 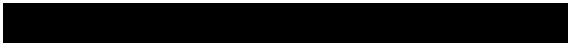 | 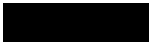 | 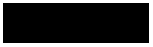 | 1.1 | 1.3 | −1.1 | 1.1 | −1 | −1.1+ |
| 211 | 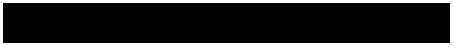 | 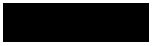 | 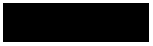 | −1.3 | −1.4 | 1.2 | [symbol] | [symbol] | 1.2 |
| 233 | 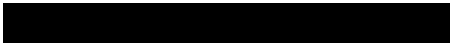 | 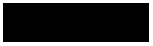 | 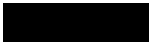 | −1.1 | −1.3 | 1.4 | −1.2 | 1.2 | −1.2 |
| 230 | 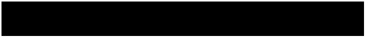 | 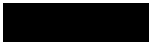 | 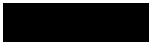 | −1.3 | −1.3 | −1.6 | −1.7 | −1.5 | −1.3* |
| 42 | AI386058_serum_albumin_variant | 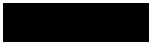 | 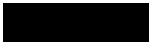 | −9.3 | −10 | −37 | −32 | −19 | −32 |
| 124 | AA067003_glutathione_S-transferase,_mu_I | 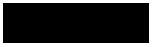 | 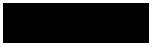 | −1.2 | 1.6 | 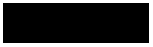 | 1.2 | −1.3 | −2.6 |
| 214 | AA261489_hypothetical_protein,_154 | 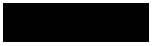 | 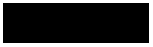 | −2.4 | [symbol] | 1.2 | 1.1 | 1.2 | 1.1 |
| 111 | AA230638_transcription_factor-like_protein_ODA-10 | 1.6 | 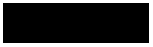 | −1.4 | −1.2 | −1.4 | −1.2 | 1.1 | −1.7 |
| 195 | AI386062_carbonic_anhydrase_3 | 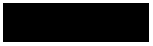 | 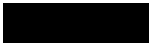 | −5.7 | −3.7 | −14 | −15 | −5.9 | −2.4 |
| 209 | 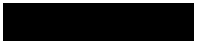 | 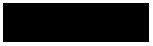 | 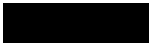 | 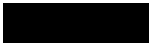 | 1 | 1.3 | 1.2 | 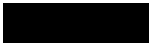 | 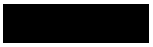 |
| 215 | 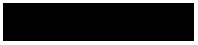 | 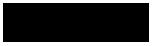 | 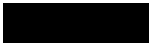 | 1.6 | 1.1 | 1.5 | −1.1 | 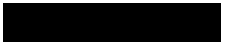 | 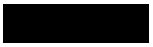 |
| 22 | 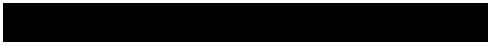 | 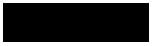 | 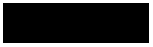 | −2.1 | −1.3 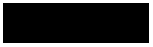 | [symbol] | 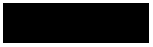 | 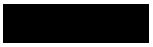 | |
| 220 | 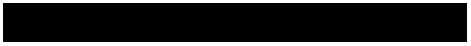 | 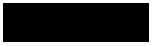 | 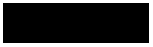 | −2.4 | −1.8 | −2.6 | −2.8 | 1 | 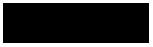 |
| 171 | AA647336_cytochrome_P450,_steroid_inducible_3a11 | 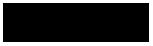 | 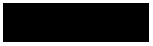 | −3.2 | −3.6 | 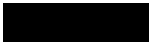 | 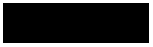 | 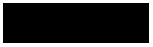 | −3.4 |
| 175 | AA237986_cytochrome_P450,_3a25 | 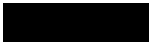 | 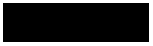 | −1.7 | −1.5 | 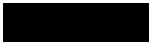 | 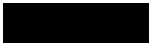 | 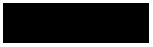 | 1.1 |
| 186 | 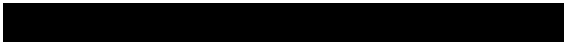 | 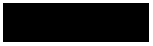 | 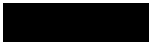 | 1.4 | 1.1* | 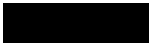 | 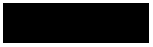 | 1 | −1.1 |
| 206 | AA822117_UDP- glucuronosyltransferase_1_family,_member_1 | 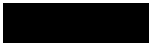 | 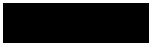 | −1.4 | −1 | 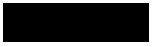 | 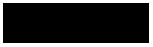 | 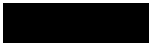 | 1.5 |
| 21 | 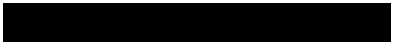 | 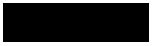 | 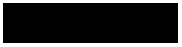 | −1.5 | 1.6 | 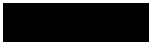 | 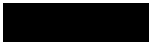 | 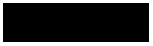 | 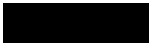 |
| 143 | AA510906_Mus_musculus_Cyp4v_mRNA_for_family_4_cyto- chrome_P450,_complete_cds | 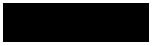 | 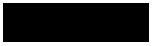 | −1.2 | −1.1 | 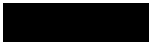 | 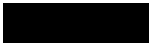 | 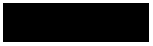 | 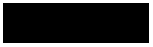 |
| 249 | AA290390_cell_death- inducing_DNA_fragmentation_factor,_alpha_subunit- like_effector_B | 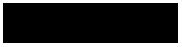 | 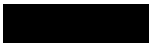 | −1.8 | −2.1 | 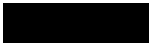 | 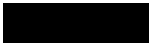 | 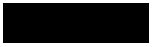 | 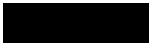 |
| 134 | AA895199_ketohexokinase | 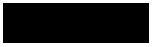 | 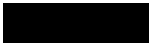 | −2.6 | −2.6 | 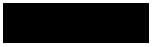 | 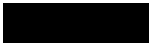 | 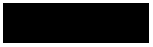 | −1.2 |
| 241 | AA237317_solute_carrier_family_2_(facilitated_glucose_trans- porter),_member_2 | 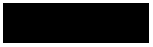 | 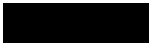 | −1.7 | −1.8 | 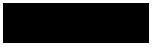 | 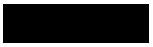 | [symbol] | −1.4+ |
| 198 | AI507919_alcohol_dehydrogenase_1,_complex | 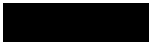 | 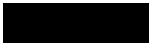 | −1.4 | −1.9 | 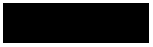 | 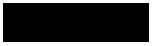 | 1.5 | 1.2* |
| 184 | AA108417_insulin-like_growth_factor_binding_protein_4 | 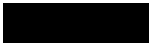 | 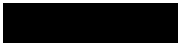 | −3.1 | −2.6 | −4.7 | −4.3 | −4 | −3.1 |
| 185 | AA145454_insulin-like_growth_factor_binding_protein_4 | 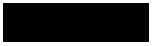 | 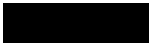 | −3 | −2.4 | −4.2 | −3.7 | −3.3 | −3.3 |
| 193 | 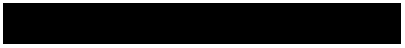 | 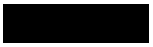 | 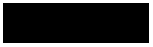 | −1.9 | −2.8 | −2.3 | −2.7 | −1.2 | −2.7 |
| 33 | 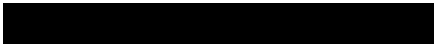 | 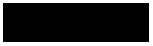 | 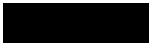 | −3 | −3.4 | −3 | −4 | −2.3 | −3.9 |
| 34 | 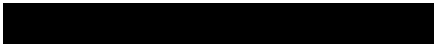 | 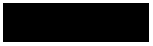 | 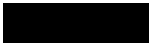 | −3.6 | −4.8 | −3.2 | −4.3 | −2.8 | −2.8 |
| 229 | AA245848_hydroxysteroid_dehydrogenase-3,_delta<5>-3-beta | 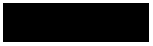 | 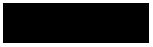 | −2.7 | −2.3 | 1.2 | −1 | −1 | 1.5 |
| 430 | AA096985_cytochrome_P450,_2j5 | 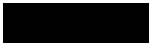 | 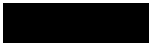 | 1.5 | −1.4 | 1.1 | −1.5 | −1.2 | −1.8+ |
| 227 | 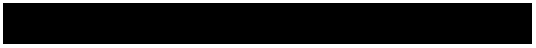 | 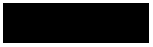 | 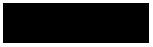 | 1.1 | 1.1 | 1.3 | 1.1 | 1.2 | 1.4 |
| 199 | 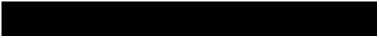 | 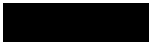 | 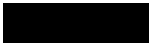 | −1.8 | −2 | −1.8 | −2 | −1.6 | −2.3 |
| 212 | 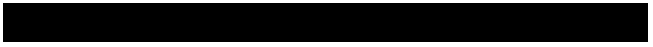 | 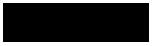 | 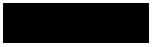 | −2.2 | −2 | −2.7 | −2.1 | −2.6 | −2.1 |
| 228 | AA027607_hydroxysteroid_17-beta_dehydrogenase_2 | 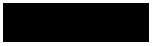 | 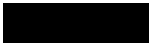 | 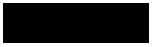 | 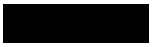 | 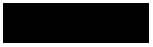 | 1.1+ | 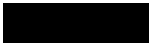 | 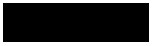 |
| 217 | AA108741_RIKEN_cDNA_0610025119_gene | 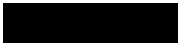 | 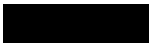 | 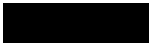 | 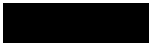 | 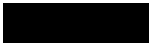 | 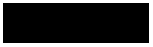 | 1.1 | 1.1 |
| 139 | AA982227_glutathione_S-transferase,_alpha_3 | 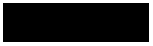 | 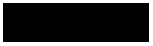 | 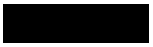 | 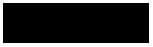 | 1.1 | 1.1 | −1.3 | −2.8 |
| 223 | AI597312_hydroxysteroid_dehydrogenase-1_delta<5>-3-beta | 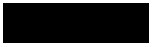 | 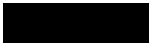 | −2 | −1.7 | −1.3 | −1.8 | −1 | −1.5 |
| 80 | 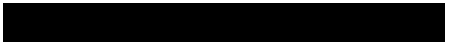 | −1.1 | −1.3 | −1.5 | −1.3 | −2.1 | −1.9 | −2.4 | −2.2 |
| 147 | AA982240_advanced_glycosylation_end_product- | −1.4 | −1.1 | 1 | −1.4 | −1.1 | −1.4 | −1.1 | −2.3 |
| | specific_receptor | | | | | | | | |
| | | 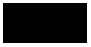 | | 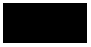 | | 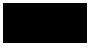 | | 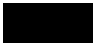 | |
| | | (−) | (+) | (−) | (+) | (−) | (+) | (−) | (+) |
| IDI | Description | PL-7B_BDE | PL-19B_BDE | PL-1B_BDE | PL-13B_BDE | PL-9B_BDE | PL-21B_BDE | PL-10B_BDE | PL-22B_BDE |
| 168 | AA756136_actin_,_gamma_2,_smooth_muscle_enteric | −2 | −2.4 | −3.4 | −3.4 | 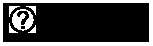 | 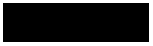 | −5 | −5.3 |
| 166 | W54383_glutathione_S-transferase,_mu_2 | −1.1 | −1.2 | −4.2 | −3.1 | 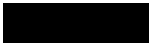 | 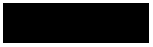 | −1 | −1.3 |
| 161 | 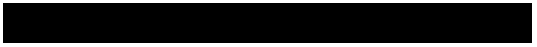 | −1.6 | −2 | −2.4 | −3.1 | 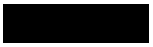 | 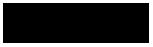 | −1.5 | −1.9 |
| 44 | AA445731_heat_shock_protein_cognate_70,_testis | 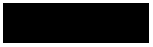 | −1.2 | −2 | −1.1 | 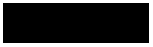 | 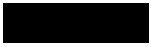 | 1.4 | 1.3 |
| 286 | W53916_RIKEN_cDNA_3300001P10_gene | −1.2 | −1.4 | −4.5 | −3.8 | 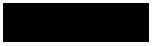 | 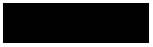 | −3.3 | −3.4 |
| 47 | 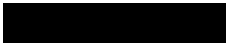 | 1.3− | −1.2+ | 1.4 | −1.5 | 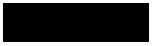 | 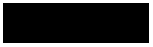 | 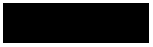 | −1.1 |
| 252 | 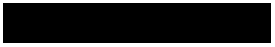 | −1.7 | −1.4 | −1.2 | −1.5 | 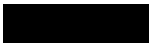 | 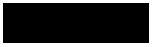 | 1.6 | 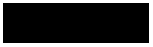 |
| 98 | 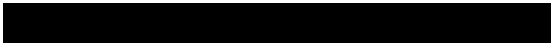 | 1.5 | −1 | 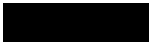 | 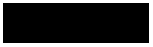 | −1.1 | −1.6 | −1.2 | −1.3 |
| 94 | 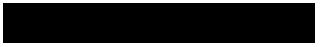 | 1.2 | 1.1 | 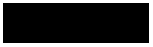 | 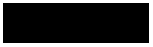 | −1 | −1 | −1.4 | 1.1 |
| 104 | 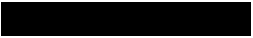 | −1.7+ | −1.6 | 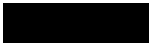 | 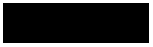 | −1.5+ | −1.7+ | −1.8 | −1.5 |
| 122 | 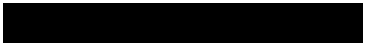 | 1.9 | 1.1 | 1.4 | 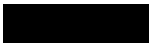 | −1.7 | −1.1 | −1.6 | −1.2 |
| 115 | 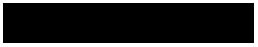 | −1.6 | 1.5 | 1.4 | 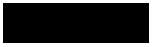 | −2.3 | −1.1 | −1.6 | −1.1 |
| 118 | 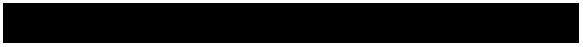 | −1.3 | 1.4 | [symbol] | 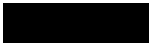 | −1.3 | 1.2 | −1.6 | −1.2 |
| 116 | 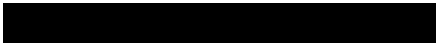 | −1.2 | 1.5 | 1.5 | [symbol] | −1.2 | 1.1 | −1.3 | −1.1 |
| 128 | 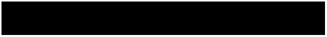 | −1.5 | 1.3 | 1.4 | 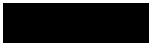 | −1.3 | 1.1 | −1.1 | 1.1 |
| 189 | 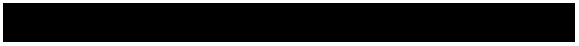 | −1.2 | −1.7 | 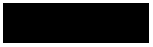 | 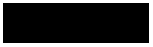 | −1.3 | −1.6 | −1.4 | −1.4 |
| 235 | 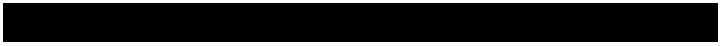 | −3.8 | −3.8 | 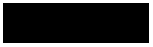 | 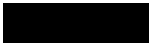 | −1.7 | −2.2 | 1.6 | 1.2 |
| 269 | 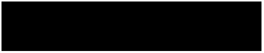 | −3.9 | −3.1 | 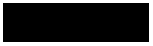 | 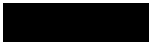 | −3.3 | −2.6 | −4.4 | −5 |
| 262 | 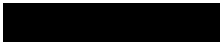 | −4.1 | −2.5 | 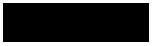 | 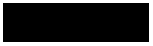 | −3.1 | −2.1 | −5.2 | −4.3 |
| 263 | 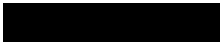 | −3.1 | −2.3 | 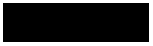 | 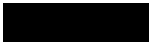 | −2.5 | −1.9 | −4.1 | −4.2 |
| 278 | AA107928_reduced_expression_3 | −2 | −1.4 | 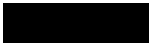 | 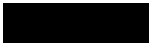 | −2.3 | −1.9 | −1.5 | −1.1 |
| 283 | AI510089_ESTs | −2 | −2.8 | 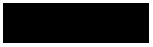 | 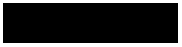 | −3.6 | −3.6 | [symbol] | −3 |
| 50 | 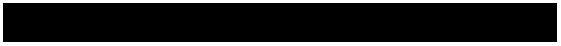 | −1.1 | −1.2 | 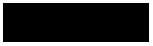 | 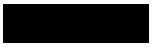 | 1.1 | −1.3 | 1.1 | −1.1 |
| 58 | 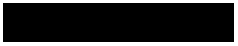 | −1.8 | 1.1 | 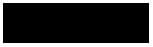 | 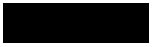 | −1.4 | −1.1 | −2.5 | −2 |
| 337 | 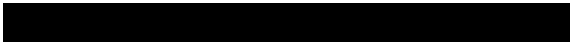 | −1.4 | −1.9 | 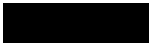 | 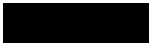 | 1 | −1.4 | −1.9 | −2 |
| 333 | 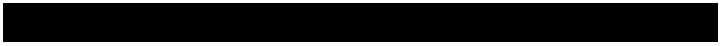 | −1.5 | −1.8 | 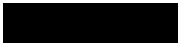 | 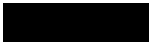 | −1.1 | −1.4 | −1.7 | −1.5 |
| 357 | 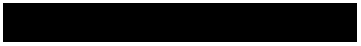 | −1.1 | −1.2 | 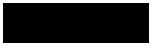 | 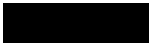 | 1.2 | −1.1 | −1.1 | −1.3 |
| 309 | 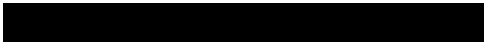 | −1.1 | 1.1 | 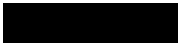 | 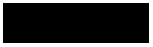 | −1.4 | −1.1 | −1.7 | −1.5 |
| 305 | 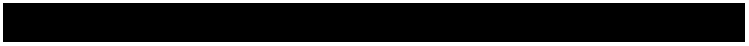 | −1.1 | −1.4 | 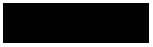 | 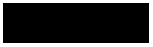 | 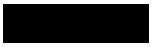 | 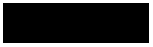 | −1.3 | −1.6 |
| 155 | 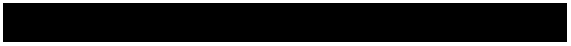 | −1.7 | −1.9 | 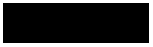 | 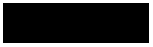 | −1.6 | −1.6 | 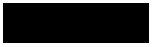 | 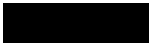 |
| 292 | 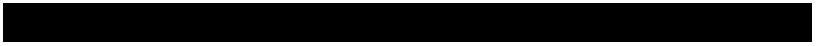 | −1.2 | 1.6 | 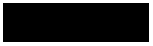 | 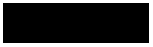 | −1.1 | 1.4 | 1.1 | 1.1 |
| 120 | 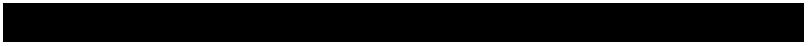 | −3.3 | −2.4 | −9.4 | [symbol] | −6.1 | −4.8 | [symbol] | −9.3 |
| 202 | 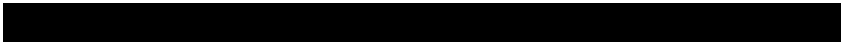 | −6.4 | −3.6 | −6.6 | −5 | −8.3 | −5.5 | −6.1 | −4.8 |
| 196 | 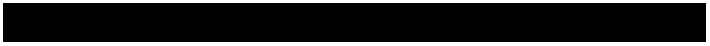 | −1.5 | [symbol] | −2.6 | −1.6 | −1.4 | −1.4 | −1.1 | −1.3 |
| 253 | 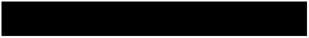 | −3.5 | −3.6 | −1.2 | −2.7 | −4.1 | −3.7 | −3.8 | −3.4 |
| 250 | 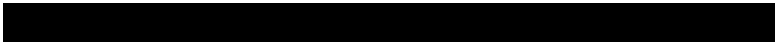 | −32 | −24 | −45 | −21 | −31 | −30 | −34 | −31 |
| 277 | AA754922_stearyl-Coenzyme_A_desaturase_1 | −2.8 | −2.3 | −4* | −3.8 | −2.9 | −2.9 | −4.6 | −3.9 |
| 290 | AA792297_procollagen,_type_V,_alpha_1 | −1.5 | −1.5 | −2.3 | −1.6 | 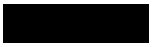 | 1.5 | −1.9 | −2.1 |
| 197 | AA474336_synaptonemal_complex_protein_3 | −2.7 | 1.3 | 1.3 | 1.3 | −2.7 | −1.3 | −1.8 | −1.3 |
| 154 | AI594147_beta-2_microhlobulin | 1.2 | [symbol] | −7.9 | −7.5 | −1.3 | −1.3 | −1.6 | −2.2 |
| 164 | AA109873_RIKEN_cDNA_0610010E05_gene | −2.5 | −2.6 | −3.1 | −3.2 | −1.9 | −2.5 | 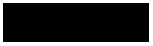 | 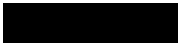 |
| 401 | AA068624_hydroxyacid_oxidase_(glycolate_oxidase)_3 | 1.1 | −1.3 | −1.2 | −1.5 | 1.1 | −1.1 | −13 | −15 |
| 151 | AI323162_dipeptidase_I_(renal) | 1.3 | 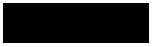 | −3.1 | −2.1 | 1.1 | 1.2 | 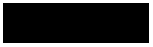 | 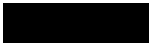 |
| 152 | 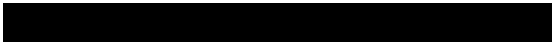 | 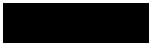 | 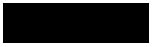 | −1.1 | −1.4 | 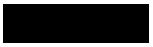 | 1.4 | −1.5 | −1.8 |
| 156 | AA793433_glutamyl_aminopeptidase | −1 | −1.1 | −2.1 | −3.6 | 1.1 | −1.3 | 1.1 | 1.1 |
| 181 | 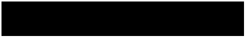 | −1.8 | −1.5 | −1.6 | −1.9 | −2.2 | −2.1 | −2.9 | −2.4 |
| 107 | 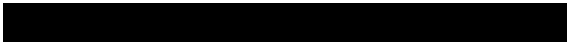 | −2.9 | [symbol] | −2.4 | −1.9 | −2.4 | −1.5 | −4.2 | −2.2 |
| 129 | 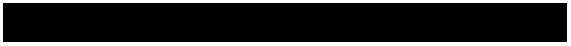 | −2.2 | −1.4 | −1.7 | −1.9 | −2 | −1.5 | −2.7 | −1.8 |
| 160 | 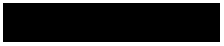 | 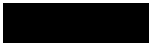 | 1.3 | −1.5 | −1.6 | 1.4 | 1.3 | 1.1 | −1 |
| 17 | 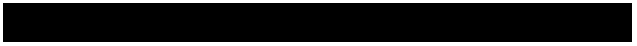 | −4.7 | −4.7 | −10* | −4.5 | −3.2 | −4.5 | −1.8 | −5.3 |
| 19 | 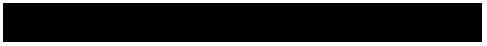 | −3 | −4.5 | −4.2 | −3.3 | −3.2 | −3.5 | −4.2 | −4.2 |
| 136 | 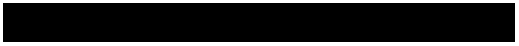 | −12 | −10 | −9.3 | −9.8 | −10 | −12 | −22 | −11 |
| 112 | 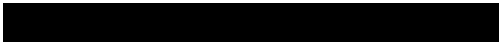 | −3.3 | −2.8 | −2.7 | −2.3 | −4 | −3.6 | −2.7 | −2.3 |
| 25 | 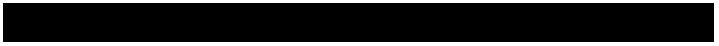 | −2.6 | −3.8 | −2.5* | −3.1 | −2.4 | −2.9 | −3.4 | −3.8 |
| 26 | 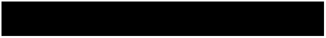 | −3 | −2.3 | −3.8 | −2 | −2.1 | −3.3 | −2.6 | −3 |
| 27 | 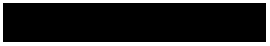 | −1.6 | −2 | −3.4 | −1.8 | −1.3 | −1.5 | −2 | −2 |
| 163 | 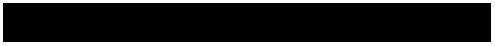 | −11 | −9.6 | −8.6 | −8.3 | −11 | −13 | −12 | −11 |
| 177 | 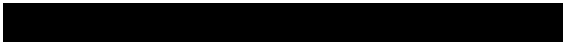 | 1.5 | 1.2 | −2.2 | −1.9 | −1.9 | −1.3 | 1.4 | 1.3 |
| 167 | 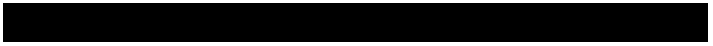 | 1 | 1 | 1.4 | 1.1 | 1.2 | −1 | −1 | −1.2 |
| 178 | 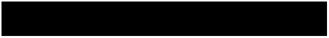 | −1.2 | −1.1 | −1.9 | −1.2 | −1 | 1.2 | −1.2 | −1.1 |
| 174 | 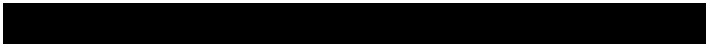 | −9.3 | −6.3 | −7 | −5.8 | −8 | −7.3 | −11 | −7.3 |
| 183 | AA481911_Mus_musculus,_clone_MGC:6227,_mRNA_com- | −7.4 | −3.1 | −22 | −14 | −21 | −15 | −3.6 | −13 |
| | plete_cds | | | | | | | | |
| 226 | AA237607_pyruvate_kinase_liver_and_red_blood_cell | −2.8 | −2.1 | −3.9 | −1.7 | −1.6 | −2 | −1.5 | −1.4 |
| 257 | AA242102_villin | −4.9 | −3.9 | −5.6 | −3.5 | −2.8 | −1.6 | 1.5 | 1.2 |
| 38 | 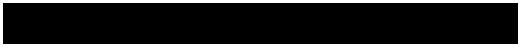 | −13 | −9.9 | −14 | −13 | −17 | −18 | −18 | −15 |
| 60 | 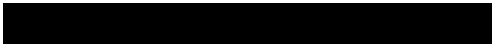 | −12 | −9.6 | −14 | −10 | −12 | −13 | −14 | −12 |
| 62 | 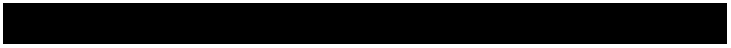 | −5 | −5.9 | −4.5 | −5.3 | −4.6 | −7.4 | −6.6 | −7.1 |
| 64 | AA146478_M_musculus_mRNA_)3C10)_for_IgA_V- | −3.5 | −2.4 | −4.9 | −4.6 | −2.8 | −4.3 | −2.8 | −5.6 |
| | D-J-heavy_chain | | | | | | | | |
| 12 | AA445667_RIKEN_cDNA_4632401C08_gene | −2.6 | −2.2 | −1.6 | −1.9 | −1.1 | −2.2 | 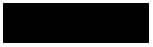 | 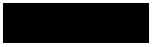 |
| 148 | AA023491_RIKEN_cDNA_1600012D06_gene | −1.2 | −1.4 | −1.1 | −1.4 | −1 | −1.4 | −1.2 | −1.1 |
| 106 | 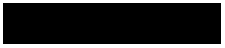 | −1.6+ | −1.7 | 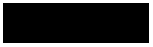 | 1.6 | −1.4 | −1.7 | 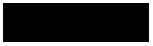 | 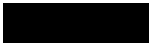 |
| 280 | AI614443_3′-phosphoadenosine_5′- phosphosulfate_synthase_2 | −1.7 | −2.4 | −3.2 | −2.5 | −1.6 | −2.4 | 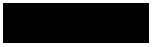 | 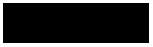 |
| 24 | AA656694_cytochrome_P450,_2b9,_phenoberbitol_in- ducible,_type_a | 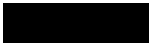 | 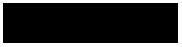 | −2.6 | −2 | −1.8 | −2.5 | −2.2 | −2.6 |
| 32 | AA325330_cytochrome_P450,_2b13,_phenoberbitol_in- ducible,_type_c | 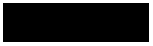 | 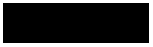 | −2.5 | −2.5 | −2.4 | −3 | −2.8 | −2.4 |
| 8 | AA107035_guanylate_cyclase_activator_2b_(retina) | −31 | −16 | −26 | −17 | −29 | −25 | −1 | −1.3 |
| 258 | W17866_urine-specific_proline-rich_acid_protein | −4.1 | −4 | −8.1 | −3.3 | −5.1 | −4 | −7.3 | −5.6 |
| 149 | AA108495_deocyribonuclease_1 | −14 | −12 | −19 | −8.3 | −6 | −10 | 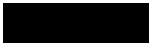 | 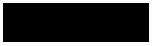 |
| 117 | AA822473_DNA_seg- ment,_Chr_8,_brighman_&_Women's_Genetics_1320_expressed | −7.7 | −4.6 | −4.8 | −2.8 | −6 | −5.2 | 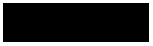 | 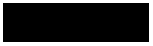 |
| 132 | AI181090_cytochrome_P450_subfamily_IV_B,_polypeptide_I | 1.3 | 1.5 | −17* | −8.7 | −11 | −8.7 | 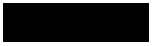 | 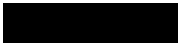 |
| 141 | AI386288_RIKEN_cDNA_2700043D08_gene | −2.1 | −1.6 | −1.7 | −1.6 | 1 | −1 | 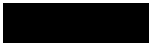 | 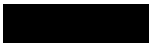 |
| 170 | 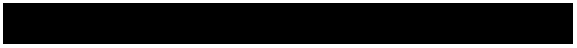 | −1.5 | −1.5 | −1.8 | −1.3 | 1.6 | 1.5 | 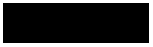 | 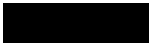 |
| 180 | 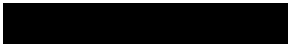 | −1.5 | −1.6 | −1.4 | −1.2 | −1.3 | −1.5 | 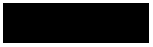 | 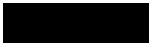 |
| 29 | 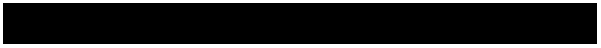 | −2.2 | −2.6 | −1.2 | −1.5 | −2 | −2.5 | 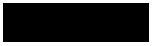 | 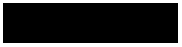 |
| 187 | 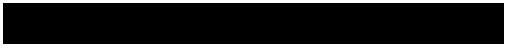 | 1 | −1 | −2 | −1.4 | −1 | −1.1 | 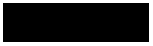 | 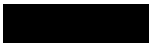 |
| 219 | 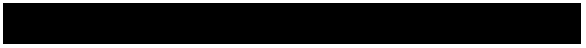 | −1 | −1.1 | −1.4 | −1.4 | −1.5 | −1.4 | 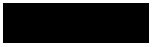 | 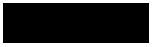 |
| 222 | 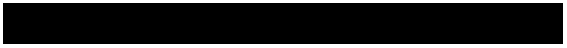 | −2.2 | −1.5 | −3.4 | −2.4 | −1.1 | −1.3 | 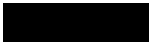 | 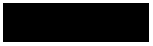 |
| 431 | AA617362_low_density_lipoprotein_receptor- related_protein_2 | −1.3 | −1.5 | −1.2+ | −1.8 | −1.2 | −1.7 | 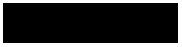 | 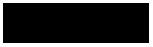 |
| 439 | AI587821_solute_carrier_family_22_(organic_cation_trans- porter),_member_2 | −1.5 | −2 | −1.8 | −1.9 | −1.3 | −1.9 | 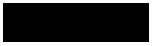 | 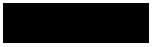 |
| 321 | AA110551_phospholipid_scramblase_2 | 1.1 | −1.1 | −3 | −2.1 | −1.3 | −2.3 | 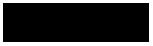 | 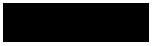 |
| 165 | AA415344_histone_deacetylase_7 | 1.1 | −1.4 | −1.2 | −2.1 | −1.1 | −1.1 | 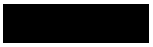 | 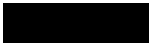 |
| 254 | W14224_N-myc_downstream_regulated_I | −4 | −4.6 | −1.8* | −2.1 | −1.2 | −2 | 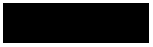 | 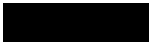 |
| 30 | 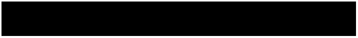 | 1.1 | 1.4 | −1.4 | −1.3 | −1.5 | −1.2 | −2.3 | −1.7 |
| 213 | 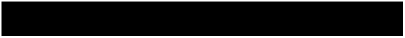 | −1.9 | −1.1 | −1.5 | −1.2 | −1.5 | −1.6 | 1.1 | −1.1 |
| 207 | 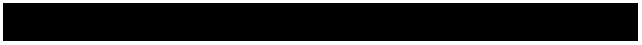 | −3.5 | 1.3 | −3 | −3.3 | −2.7 | −3.5 | −4.7 | −3.5 |
| 205 | 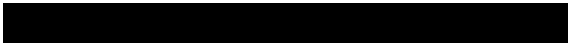 | −1.7 | 1.1 | 1* | −1.2 | −1.5 | −1.1 | 1.2 | 1.2 |
| 237 | 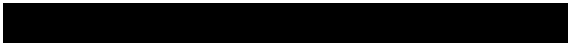 | −1.7* | −1.1 | −1.3 | −1.2 | −1.5 | −1.3+ | −1.2 | 1.1 |
| 211 | 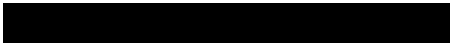 | −1.9 | −1.1 | −3.5 | −2.5 | −2.2 | −1.9 | −2 | −2.2 |
| 233 | 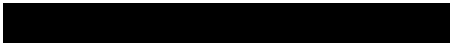 | −3 | −1.2 | −2.6 | −2.1 | −2.7 | −2.8 | −2.3 | −2.5 |
| 230 | 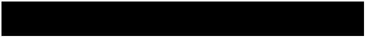 | −1.7 | −1 | −2.2 | −1.3 | −1.5 | −1.7 | −1.5 | [symbol] |
| 42 | AI386058_serum_albumin_variant | −42 | −1.8 | −44 | −33 | −48 | −33 | −54 | −35 |
| 124 | AA067003_glutathione_S-transferase,_mu_I | 1.3 | 1.3 | −1 | −1.5 | 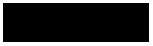 | 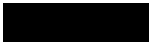 | 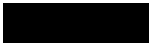 | 1.1 |
| 214 | AA261489_hypothetical_protein,_154 | −3.2 | −2.7 | 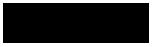 | 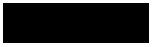 | −2.2 | −2.3 | 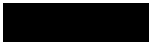 | 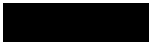 |
| 111 | AA230638_transcription_factor-like_protein_ODA-10 | −1.1 | 1.5 | −1.1 | 1.2 | 1.2 | 1.3 | −1.6 | −1.5 |
| 195 | AI386062_carbonic_anhydrase_3 | −3.9 | −1.8 | −25 | −1.7 | −1.3 | −2.8 | −2.3 | |
| 209 | 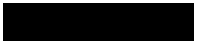 | −1.7 | 1.5 | 1.3 | 1.2 | −1.2 | 1.2 | 1.1 | 1.5 |
| 215 | 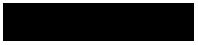 | −1.3 | 1.5 | 1.3 | 1.3 | −1.2 | 1.2 | 1 | 1.3 |
| 22 | 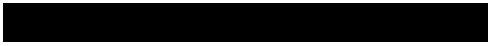 | 1 | 1.2 | −2.1 | −3.2 | −1.7 | −1.6 | 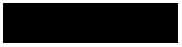 | 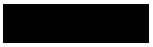 |
| 220 | 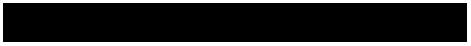 | 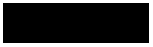 | 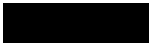 | −6.6 | −4.6 | −2.3 | −2.2 | 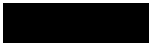 | 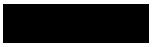 |
| 171 | AA647336_cytochrome_P450,_steroid_inducible_3a11 | −8.7 | −1.1 | −6.6 | −5 | −7.5 | −7.2 | −7.6 | −7 |
| 175 | AA237986_cytochrome_P450,_3a25 | −2.8 | −1.3 | −2.6 | −2.4 | −1.7 | −2.1 | −1.7 | −1.8 |
| 186 | 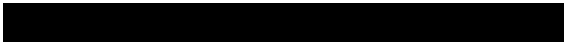 | 1 | −1.2 | −2.1 | −2 | 1.2 | 1.3 | 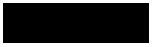 | 1.3 |
| 206 | AA822117_UDP- glucuronosyltransferase_1_family,_member_1 | −2 | −1.7 | −6.4 | −5.1 | 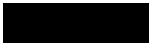 | 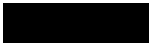 | 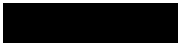 | 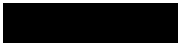 |
| 21 | 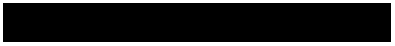 | −25 | −2.8 | [symbol] | −11 | −16 | −14 | −31 | −15 |
| 143 | AA510906_Mus_musculus_Cyp4v_mRNA_for_family_4_cyto- | −1.2 | −1.1 | −2.7 | −2.2 | −1.5 | −1.5 | −2.2 | −2.3 |
| | chrome_P450,_complete_cds | | | | | | | | |
| 249 | AA290390_cell_death- inducing_DNA_fragmentation_factor,_alpha_subunit- like_effector_B | −1.7 | −2.9 | −3.7 | −2.5 | −3.4 | −4.4 | 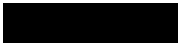 | 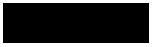 |
| 134 | AA895199_ketohexokinase | −3.5 | −2.4 | −4.4 | −2.6 | −3 | −2.8 | 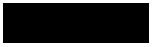 | 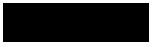 |
| 241 | AA237317_solute_carrier_family_2_(facilitated_glucose_trans- porter),_member_2 | −1.4+ | −1.7 | −1.4 | 1.4 | −1.4 | −1.9 | 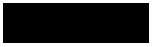 | 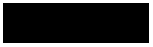 |
| 198 | AI507919_alcohol_dehydrogenase_1,_complex | −1.9 | −1.3 | −2.3 | −2.2 | 1.5 | 1.4 | 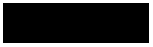 | 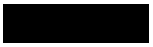 |
| 184 | AA108417_insulin-like_growth_factor_binding_protein_4 | −1.4 | −1.2 | −4.4 | −4.6 | −1.4 | −1.7 | 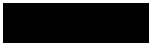 | 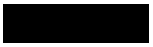 |
| 185 | AA145454_insulin-like_growth_factor_binding_protein_4 | −1.3 | −1.3 | −4.8 | −4.3 | −1.3 | −1.8 | 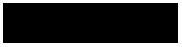 | 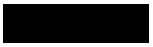 |
| 193 | 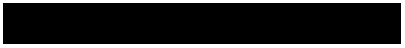 | −3.5 | −2.9 | −5.1 | −2.7 | −3 | −3.8 | 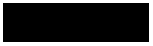 | 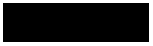 |
| 33 | 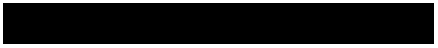 | 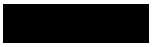 | 1.5 | −7.1 | −3.4 | −4.8 | −4.8 | 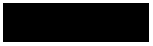 | 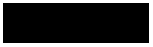 |
| 34 | 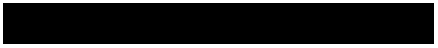 | 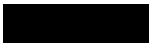 | 1.4 | −8.2 | −3.4 | −5.3 | −5.7 | 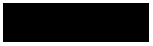 | 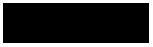 |
| 229 | AA245848_hydroxysteroid_dehydrogenase-3,_delta<5>-3-beta | −3.5 | −2.7 | −4.9 | −3.2 | −3.5 | −4.1 | 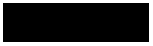 | 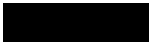 |
| 430 | AA096985_cytochrome_P450,_2j5 | −1.6 | −1.5 | 1 | −1.4 | −1.1 | −1.6 | 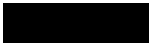 | 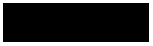 |
| 227 | 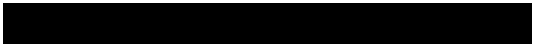 | 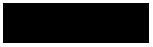 | 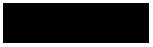 | −1.6 | −1.3 | −1.6 | −1.6 | 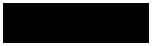 | 1.4 |
| 199 | 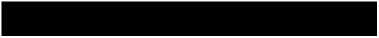 | 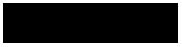 | 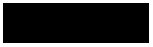 | −1.4 | −4 | −1.7 | −3.2 | 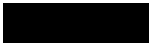 | 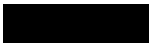 |
| 212 | 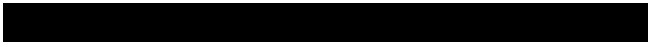 | 1.6 | 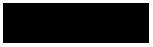 | −2.4 | −2.4 | −1.3 | −1.6 | 1.2 | 1.3 |
| 228 | AA027607_hydroxysteroid_17-beta_dehydrogenase_2 | −1.4 | −1.4 | −1.4 | −1.1 | 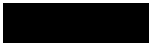 | 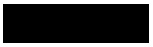 | 1.2 | −1 |
| 217 | AA108741_RIKEN_cDNA_0610025119_gene | 1.4 | 1.1 | −1.4 | −1.6 | −1.4 | −1.6 | 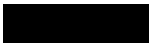 | 1.6 |
| 139 | AA982227_glutathione_S-transferase,_alpha_3 | 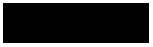 | 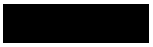 | −7.4 | −3.3 | 1.2 | −1.4 | 1.3 | −1 |
| 223 | AI597312_hydroxysteroid_dehydrogenase-1_delta<5>-3-beta | −1.6 | 1.4+*** | −3 | −3.1 | −3.2 | −2.8 | 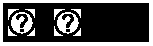 | 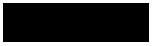 |
| 80 | 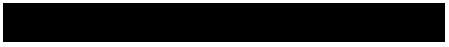 | 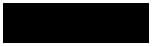 | 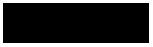 | −1.6 | −1.5 | 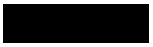 | −1.3 | 1.2 | 1.4 |
| 147 | AA982240_advanced_glycosylation_end_product- specific_receptor | 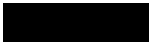 | 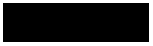 | −1.5 | −1.4 | −1.1 | −1.1 | −1.6 | −1.7 |

| | | 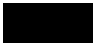 | | 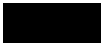 | | | | |
|---|---|---|---|---|---|---|---|---|
| | | (−) | (+) | (−) | (+) | | | |
| IDI | Description | PL-11B_BDE | PL-23B_BDE | PL-12B_BDE | PL-24B_BDE | Max Signal | Arrange Signal | Missed Values |
| 168 | AA756136_actin_,_gamma_2,_smooth_muscle_enteric | 1.4 | 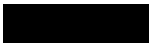 | −1 | −1.1 | [symbol] | [symbol] | 0 |
| 166 | W54383_glutathione_S-transferase,_mu_2 | −2 | −2.3 | −1.9 | −2 | [symbol] | [symbol] | 0 |
| 161 | 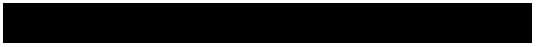 | −1.3 | −1.3 | −1.4 | −1.4 | [symbol] | [symbol] | 0 |
| 44 | AA445731_heat_shock_protein_cognate_70,_testis | −1 | 1.2 | −1.2 | −1.3 | 5558 | [symbol] | 0 |
| 286 | W53916_RIKEN_cDNA_3300001P10_gene | −2.7 | −3.1 | 1.3 | 1.2 | [symbol] | [symbol] | 0 |
| 47 | 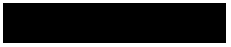 | 1.5 | −1.2+ | [symbol] | −1.2+ | 1152 | 258 | [symbol] |
| 252 | 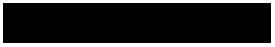 | −1.8 | −1.5 | 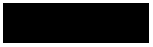 | 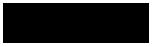 | [symbol] | [symbol] | 0 |
| 98 | 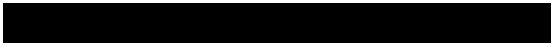 | 1 | [symbol] | −1.1 | −1.3 | [symbol] | [symbol] | 1 |
| 94 | 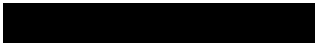 | −1.2 | −1.4 | −1.1 | −1.2 | [symbol] | [symbol] | 0 |
| 104 | 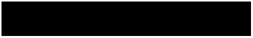 | −1.4 | −1.2+ | −1.4 | −1.2+ | 876 | [symbol] | 12 |
| 122 | 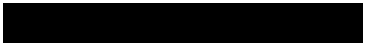 | −1 | 1.6 | −1.1 | 1.1 | [symbol] | [symbol] | 0 |
| 115 | 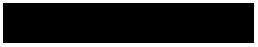 | −1.7 | 1.5 | −1.5 | −1.1 | [symbol] | [symbol] | 0 |
| 118 | 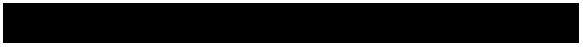 | −1.3 | 1.2 | −1.5 | −1.2 | [symbol] | [symbol] | 0 |
| 116 | 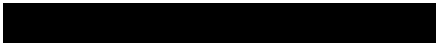 | −1.3 | 1.4 | −1.1 | 1 | 4719 | [symbol] | 0 |
| 128 | 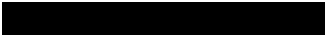 | −1.1 | [symbol] | −1 | 1.1 | 3167 | 1778.75 | 0 |
| 189 | 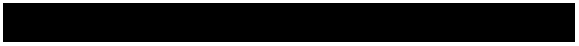 | −1.1 | −1.5 | 1.3 | 1.2 | [symbol] | [symbol] | 0 |
| 235 | 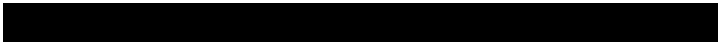 | −1.4 | −1.9 | −1.1 | −1.7 | [symbol] | [symbol] | 0 |
| 269 | 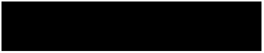 | −1.9 | −1.8 | −2.3 | −2.6 | [symbol] | [symbol] | 0 |
| 262 | 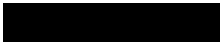 | −1.9 | −1.5 | −2.4 | −2.1 | 2357 | [symbol] | 0 |
| 263 | 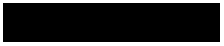 | −1.8 | −1.5 | −2.4 | −2.2 | [symbol] | [symbol] | 0 |
| 278 | AA107928_reduced_expression_3 | 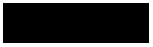 | [symbol] | −1.9 | −1.4 | [symbol] | [symbol] | 1 |
| 283 | AI510089_ESTs | −2.4 | −2.3 | −2.6 | −2 | [symbol] | [symbol] | 0 |
| 50 | 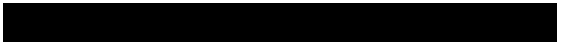 | 1.1 | −1.3 | 1.2 | −1.2 | [symbol] | [symbol] | 2 |
| 58 | 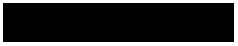 | −1.2 | 1.4 | −1.5 | −1.5 | [symbol] | [symbol] | 0 |
| 337 | 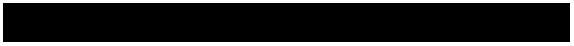 | −1.2 | −1.7 | −1.2 | −1.6 | [symbol] | [symbol] | 0 |
| 333 | 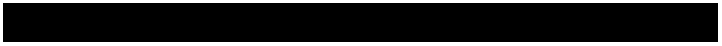 | −1.1 | −1.4 | −1.1 | −1.4 | [symbol] | [symbol] | 1 |
| 357 | 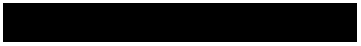 | 1.2 | 1 | 1.1 | −1.2 | [symbol] | [symbol] | 1 |
| 309 | 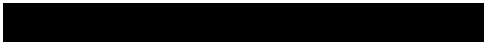 | −1.4 | −1.3 | −1.5 | −1.8 | [symbol] | [symbol] | 0 |
| 305 | 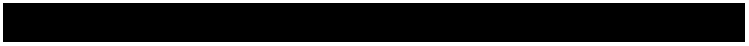 | 1.2 | −1* | 1.2 | 1.1 | [symbol] | [symbol] | 0 |
| 155 | 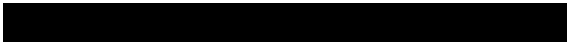 | −1.7 | −1.4 | −1.2 | −1 | [symbol] | [symbol] | 0 |
| 292 | 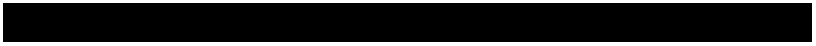 | 1.4 | 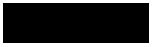 | −1 | −1.2 | [symbol] | [symbol] | 3 |
| 120 | 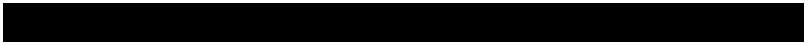 | −8.1 | −6.7 | 1.4 | 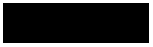 | [symbol] | [symbol] | 0 |
| 202 | 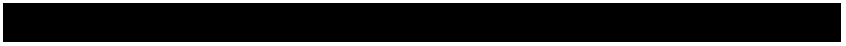 | −9.5 | −11 | 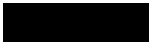 | 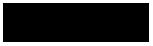 | [symbol] | [symbol] | 0 |
| 196 | 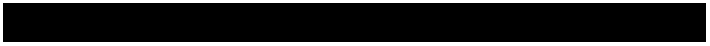 | −1.4 | −2.1 | 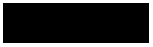 | 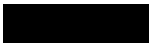 | [symbol] | [symbol] | 0 |
| 253 | 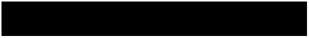 | −3.4 | −1.4 | 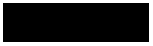 | 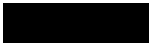 | [symbol] | 719.375 | 0 |
| 250 | 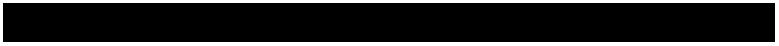 | −18 | −22 | 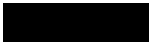 | 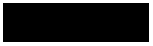 | [symbol] | [symbol] | 0 |
| 277 | AA754922_stearyl-Coenzyme_A_desaturase_1 | −2.4 | −4.9 | 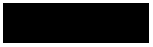 | 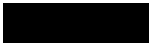 | [symbol] | 211.579 | 0 |
| 290 | AA792297_procollagen,_type_V,_alpha_1 | 1.1 | 1.3 | 1.1 | 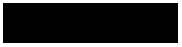 | [symbol] | [symbol] | 0 |
| 197 | AA474336_synaptonemal_complex_protein_3 | −2 | 1.1 | −2.3 | −1.7 | [symbol] | [symbol] | 0 |
| 154 | AI594147_beta-2_microhlobulin | 1 | 1.1 | 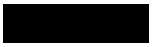 | 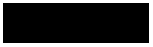 | [symbol] | [symbol] | 0 |
| 164 | AA109873_RIKEN_cDNA_0610010E05_gene | −1.1 | 1 | −2.3 | −2.1 | [symbol] | [symbol] | 0 |
| 401 | AA068624_hydroxyacid_oxidase_(glycolate_oxidase)_3 | 1.1 | −1.2 | 1.1 | −1.3 | [symbol] | [symbol] | 0 |
| 151 | AI323162_dipeptidase_I_(renal) | −1.5 | [symbol] | 1.2 | 1.5 | [symbol] | [symbol] | 0 |
| 152 | 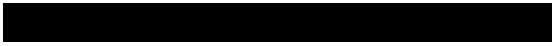 | 1.2 | 1 | 1.3 | 1.6 | [symbol] | [symbol] | 1 |
| 156 | AA793433_glutamyl_aminopeptidase | 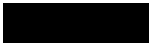 | 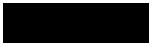 | 1.3 | 1.5 | [symbol] | [symbol] | 0 |
| 181 | 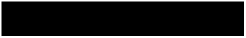 | 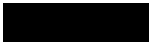 | 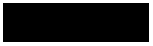 | −1.4 | −2.4 | 5079 | [symbol] | 0 |
| 107 | 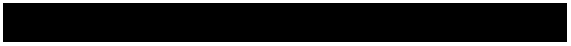 | −2.9 | −1.9 | −3.7 | −2.2 | [symbol] | [symbol] | 0 |
| 129 | 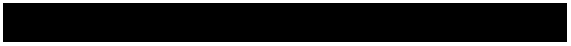 | −2.2 | −1.8 | −2.4 | −2.1 | [symbol] | [symbol] | 0 |
| 160 | 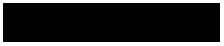 | 1.2 | 1.1 | 1 | 1.1 | [symbol] | [symbol] | 0 |
| 17 | 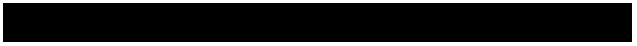 | [symbol] | −5.1 | −3.2 | −6.5 | [symbol] | [symbol] | 0 |
| 19 | 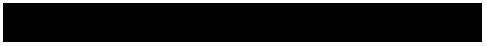 | −2.3 | −4.5 | −2.5 | −3.6 | 21576 | [symbol] | 0 |
| 136 | 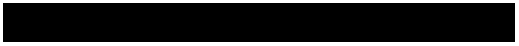 | −10 | −11 | −11 | −11 | 21549 | [symbol] | 0 |
| 112 | 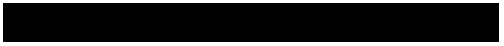 | −3.4 | −2.9 | −3.5 | −2.4 | [symbol] | [symbol] | 0 |
| 25 | 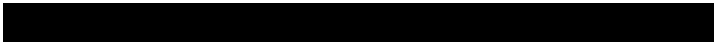 | −1.9 | −3.6 | −2 | −3.2 | [symbol] | [symbol] | 0 |
| 26 | 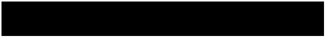 | −2 | −2.6 | −2 | −3.4 | [symbol] | [symbol] | 0 |
| 27 | 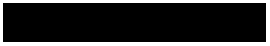 | −1.4 | −2 | −1.4 | −2.5 | [symbol] | [symbol] | 0 |
| 163 | 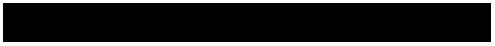 | −9.1 | −10 | −9.9 | −10 | [symbol] | [symbol] | 0 |
| 177 | 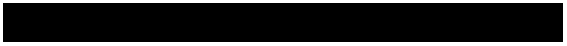 | 1.2 | 1.2 | −1.1 | −1.5 | [symbol] | [symbol] | 0 |
| 167 | 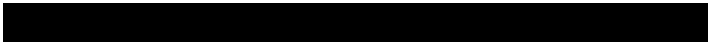 | 1 | −1 | −1.1 | [symbol] | [symbol] | [symbol] | 0 |
| 178 | 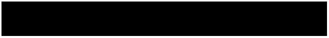 | −1.1 | −1.2 | −1 | −1.5 | 5741 | 1857 | 0 |
| 174 | 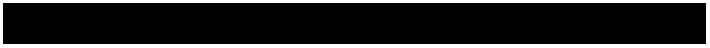 | −7.2 | −7.9 | −6.3 | −7.3 | [symbol] | [symbol] | 0 |
| 183 | AA481911_Mus_musculus,_clone_MGC:6227,_mRNA_com- | −3.8 | −2.8 | −5 | −3.7 | [symbol] | [symbol] | 0 |
| | plete_cds | | | | | | | |
| 226 | AA237607_pyruvate_kinase_liver_and_red_blood_cell | −1.5 | −1.5 | −1.9 | −2.1 | 5887 | [symbol] | 0 |
| 257 | AA242102_villin | −2.5 | −3.2 | −2.4 | −5.8 | [symbol] | [symbol] | 0 |
| 38 | 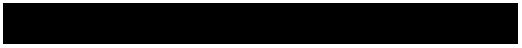 | −14 | −18 | −12 | −13 | [symbol] | [symbol] | 0 |
| 60 | 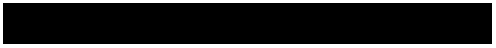 | −9.2 | −13 | −8.5 | −11 | [symbol] | [symbol] | 0 |
| 62 | 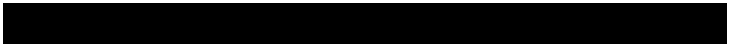 | −4.3 | −7.2 | −1.6 | −5.7 | [symbol] | [symbol] | 0 |
| 64 | AA146478_M_musculus_mRNA_)3C10)_for_IgA_V- | −2 | −2.3 | −2.4 | −3.9 | [symbol] | [symbol] | 0 |
| | D-J-heavy_chain | | | | | | | |
| 12 | AA445667_RIKEN_cDNA_4632401C08_gene | −1.4 | −2.5 | −1.3 | −2.6 | [symbol] | [symbol] | 0 |
| 148 | AA023491_RIKEN_cDNA_1600012D06_gene | [symbol] | 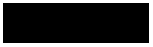 | −1 | −1.4 | [symbol] | [symbol] | 0 |
| 106 | 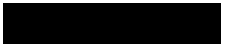 | −1.8 | −2 | −2.2 | −1.6+ | 2775 | [symbol] | 5 |
| 280 | AI614443_3′-phosphoadenosine_5′- | −1.5 | −3.1 | −1.5 | −2.6 | 8759 | 1253.54 | 0 |
| | phosphosulfate_synthase_2 | | | | | | | |
| 24 | AA656694_cytochrome_P450,_2b9,_phenoberbitol_in- | −2.2 | −2.4 | −2.2 | −2.6 | 3580 | 787.125 | 0 |
| | ducible,_type_a | | | | | | | |
| 32 | AA325330_cytochrome_P450,_2b13,_phenoberbitol_in- | −2.4 | −2.7 | −1.9 | −2.6 | 2277 | [symbol] | 1 |
| | ducible,_type_c | | | | | | | |
| 8 | AA107035_guanylate_cyclase_activator_2b_(retina) | 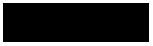 | 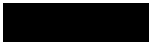 | −3.6 | −24 | [symbol] | [symbol] | 0 |
| 258 | W17866_urine-specific_proline-rich_acid_protein | 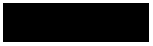 | 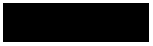 | −1.9 | −5.5 | [symbol] | [symbol] | 0 |
| 149 | AA108495_deocyribonuclease_1 | −9.7 | −13 | −9.4 | −15 | [symbol] | [symbol] | 0 |
| 117 | AA822473_DNA_seg- | −6 | −1.9 | −4.9 | −1.7 | [symbol] | [symbol] | 0 |
| | ment,_Chr_8,_brighman_&_Women's_Genetics_1320_expressed | | | | | | | |
| 132 | AI181090_cytochrome_P450_subfamily_IV_B,_polypeptide_I | −13 | −12 | −5.9 | −5.5 | [symbol] | [symbol] | 0 |
| 141 | AI386288_RIKEN_cDNA_2700043D08_gene | −1.1 | 1 | −1.1 | −1.1 | [symbol] | 1459.58 | 0 |
| 170 | 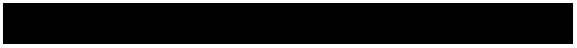 | 1.3 | 1.5 | −1.3* | −1.5 | [symbol] | [symbol] | 0 |
| 180 | 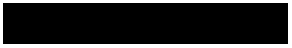 | 1 | 1.2 | −1.2 | −1.8 | 1553 | [symbol] | 2 |
| 29 | 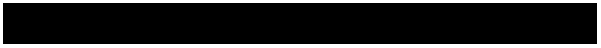 | −1.5 | −2.2 | −1.5 | −2.9 | 8575 | 833.458 | 0 |
| 187 | 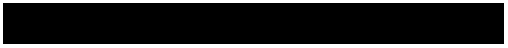 | 1 | 1.1** | 1.1 | 1.1 | [symbol] | [symbol] | 0 |
| 219 | 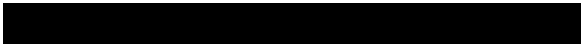 | 1.5 | 1.4 | 1.3 | [symbol] | [symbol] | [symbol] | 0 |
| 222 | 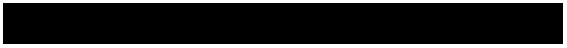 | −1.9 | −1.9 | −1.5 | −1.6 | [symbol] | [symbol] | 0 |
| 431 | AA617362_low_density_lipoprotein_receptor- | 1.1 | 1 | −1.2 | −2 | [symbol] | [symbol] | 2 |
| | related_protein_2 | | | | | | | |
| 439 | AI587821_solute_carrier_family_22_(organic_cation_trans- | −1.2 | −1.6 | −1.2 | −1.2 | [symbol] | [symbol] | 0 |
| | porter),_member_2 | | | | | | | |
| 321 | AA110551_phospholipid_scramblase_2 | −1.3 | −1.6 | 1.5 | 1.2 | [symbol] | [symbol] | 0 |
| 165 | AA415344_histone_deacetylase_7 | 1.5 | 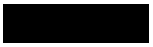 | −1.2* | −1.2 | 2245 | [symbol] | 0 |
| 254 | W14224_N-myc_downstream_regulated_I | 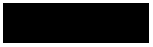 | 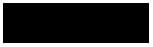 | 1.6 | −1.4 | [symbol] | [symbol] | 0 |
| 30 | 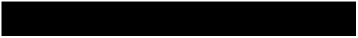 | −1.1 | −1.4 | −1.9 | −1.8 | [symbol] | [symbol] | 0 |
| 213 | 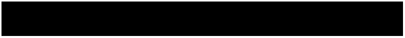 | −1.4 | −1.7 | −2 | −1.9 | [symbol] | [symbol] | 0 |
| 207 | 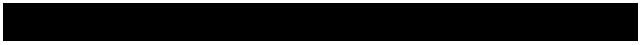 | −2.6 | −1.1 | −1.1 | −1.6 | [symbol] | [symbol] | 0 |
| 205 | 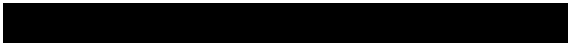 | −1.6 | −1.3 | −1.9 | [symbol] | 2217 | [symbol] | 4 |
| 237 | 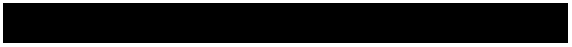 | −1.2 | 1.1 | −1.5 | −1.3+ | [symbol] | [symbol] | 7 |
| 211 | 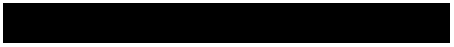 | −2.6 | −2.5 | −2 | −1.9 | [symbol] | [symbol] | 0 |
| 233 | 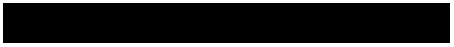 | −2.8 | −2.6 | −2.6 | −3.8 | [symbol] | [symbol] | 0 |
| 230 | 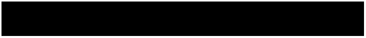 | [symbol] | −1.8 | −1.4 | −1.7 | [symbol] | [symbol] | 0 |
| 42 | AI386058_serum_albumin_variant | −12 | −9 | −21 | −7.1 | [symbol] | [symbol] | 0 |
| 124 | AA067003_glutathione_S-transferase,_mu_I | −2.9 | −2.2 | −1.9 | −1.6 | [symbol] | [symbol] | 0 |
| 214 | AA261489_hypothetical_protein,_154 | −2.1 | −2.1 | −3.3 | −3.2 | [symbol] | [symbol] | 0 |
| 111 | AA230638_transcription_factor-like_protein_ODA-10 | 1.1 | 1.4 | 1.5 | 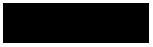 | [symbol] | [symbol] | 0 |
| 195 | AI386062_carbonic_anhydrase_3 | −17 | −11 | 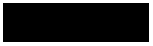 | 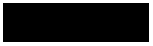 | [symbol] | [symbol] | 0 |
| 209 | 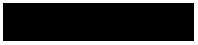 | −1.7 | 1.4 | 1.3 | 1.5 | [symbol] | [symbol] | 0 |
| 215 | 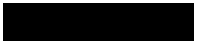 | −1.4 | 1.4 | 1.2 | 1.2 | [symbol] | [symbol] | 0 |
| 22 | 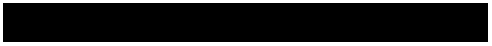 | −1.6 | −1 | −1.1 | 1.1 | 21841 | [symbol] | 0 |
| 220 | 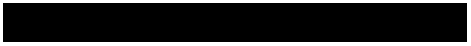 | −3.6 | −3.2 | −2.7 | −2.9 | 11761 | [symbol] | 0 |
| 171 | AA647336_cytochrome_P450,_steroid_inducible_3a11 | −5.5 | −6.4 | −8.2 | −9.1 | [symbol] | 115754 | 0 |
| 175 | AA237986_cytochrome_P450,_3a25 | −2.3 | −2.4 | −2 | −2.6 | [symbol] | [symbol] | 0 |
| 186 | 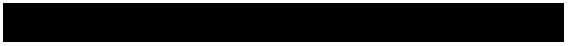 | −1.6 | −1.8 | 1.1 | 1.2 | [symbol] | [symbol] | 0 |
| 206 | AA822117_UDP- | −3.2 | −2.7 | −3 | −2.6 | [symbol] | [symbol] | 0 |
| | glucuronosyltransferase_1_family,_member_1 | | | | | | | |
| 21 | 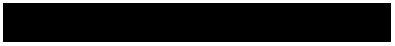 | −1.5 | 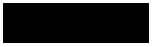 | −14 | −8.7 | [symbol] | [symbol] | 0 |
| 143 | AA510906_Mus_musculus_Cyp4v_mRNA_for_family_4_cyto- | −1.2 | −1.5 | −1.5 | −1.6 | [symbol] | [symbol] | 0 |
| | chrome_P450,_complete_cds | | | | | | | |
| 249 | AA290390_cell_death- | −2.2 | −2.6 | −2.6 | −5 | 7518 | 1374.79 | 0 |
| | inducing_DNA_fragmentation_factor,_alpha_subunit- | | | | | | | |
| | like_effector_B | | | | | | | |
| 134 | AA895199_ketohexokinase | −1.7 | −4.4 | −4 | −3.7 | 14132 | [symbol] | 0 |
| 241 | AA237317_solute_carrier_family_2_(facilitated_glucose_trans- | −1.3 | −1.8 | −1.5 | −2+ | 1071 | [symbol] | 0 |
| | porter),_member_2 | | | | | | | |
| 198 | AI507919_alcohol_dehydrogenase_1,_complex | −3.5 | −2.7 | −3.3 | −2.5 | [symbol] | [symbol] | 0 |
| 184 | AA108417_insulin-like_growth_factor_binding_protein_4 | −2.7 | −2.4 | −1.3 | 1 | [symbol] | [symbol] | 0 |
| 185 | AA145454_insulin-like_growth_factor_binding_protein_4 | −2.5 | −2.4 | −1.2 | 1 | [symbol] | [symbol] | 0 |
| 193 | 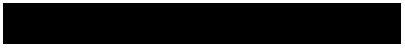 | −3.1 | −3.2 | −3.6 | −3 | [symbol] | [symbol] | 0 |
| 33 | 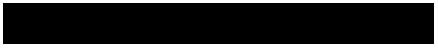 | −5.2 | −4.9 | −5.9 | −5 | [symbol] | [symbol] | 0 |
| 34 | 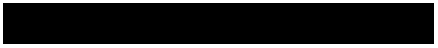 | −3 | −3.4 | −6 | −6.5 | [symbol] | [symbol] | 0 |
| 229 | AA245848_hydroxysteroid_dehydrogenase-3,_delta<5>-3-beta | −2 | −1.9 | −3.9 | −4.7 | [symbol] | [symbol] | 0 |
| 430 | AA096985_cytochrome_P450,_2j5 | −1.2 | −1.6 | −1.1 | −1.8 | [symbol] | 555.5 | 1 |
| 227 | 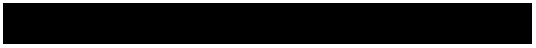 | 1 | 1.1 | −1.1 | −1.3 | 4777 | [symbol] | 0 |
| 199 | 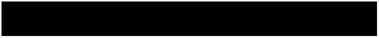 | −6.3 | −5.9 | 1.2 | [symbol] | [symbol] | [symbol] | 0 |
| 212 | 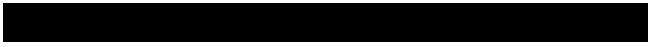 | 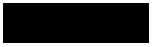 | 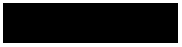 | 1.1 | 1.2 | [symbol] | [symbol] | 0 |
| 228 | AA027607_hydroxysteroid_17-beta_dehydrogenase_2 | 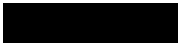 | 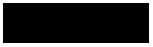 | −1.1 | −1.3 | 1473 | 378.542 | 7 |
| 217 | AA108741_RIKEN_cDNA_0610025119_gene | −1.5 | −1.8 | −1.1 | −1.1 | [symbol] | [symbol] | 0 |
| 139 | AA982227_glutathione_S-transferase,_alpha_3 | −4 | −3.4 | −2 | −2 | [symbol] | 1518.67 | 0 |
| 223 | AI597312_hydroxysteroid_dehydrogenase-1_delta<5>-3-beta | −2.4 | −1.6 | −3.9 | −4.4 | [symbol] | 585.875 | 1 |
| 80 | 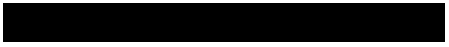 | 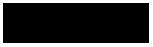 | 1.4 | 1.2 | 1.1 | [symbol] | [symbol] | 0 |
| 147 | AA982240_advanced_glycosylation_end_product- | −1.2 | −1.5 | −1.2 | −1.6 | 15915 | [symbol] | 0 |
| | specific_receptor | | | | | | | |

| | | | | | 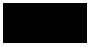 | | 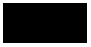 | |
|---|---|---|---|---|---|---|---|---|
| | | | | | (−) | (+) | (−) | (+) |
| IDI | Description | No. Tiss | 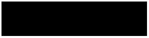 | 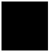 | PL-4B_BDE | PL-16B_BDE | PL-2B_BDE | PL-14B_BDE |
| 244 | AA726092_RIKEN_cDNA_4933419D20_gene | 1 | lung | lung | −1.2 | −1.3 | 1.1 | −1 |
| 289 | AA222208_surfactant_associated_protein_C | 1 | spleen | lung | −12 | −24 | −18 | −11 |
| 366 | AA710379_neuroplin | 1 | stomach | lung | 1 | 1.1+ | 1.3 | 1.1 |
| 344 | AI158775_protein_tyrosine_phosphatase,_receptor- type,_M | 1 | stomach | lung | 1.2 | 1.2+ | 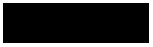 | 1.5 |
| 204 | AA929863_ESTs,_similar_to_T46271_hypo- | 1 | liver | lung, blad | −1 | −1.1 | −1.2 | −1.1 |
| | thetical_protein_DKFZp564P1263.1_[H.sapiens] | | | | | | | |
| 157 | AI892437_transglutaminase_2,_C_polypeptide | 1 | intestine | lung, blad, pl/ut | −1.5 | −1.4 | −2.5 | −2.2 |
| 203 | W18463_thioether_S-methyltransferase | 1 | liver | lung, kidn | −9.1 | −9.4 | −5.8 | −3.8 |
| 200 | AI117310_flavin_containing_monooxygenase_1 | 1 | liver | lung, kidn | −7.5 | −7.6 | −4.5 | −2.3 |
| 135 | AA670943_phosphatidic_acid_phosphotase_2a | 1 | colon | lung, pl/ut | 1.4 | 1.3 | −2.2 | −1.9 |
| 172 | AI552087_IQ_motif_containing_GTPase_activating_protein_1 | 1 | intestine | lung, pl/ut | 1.5 | 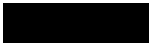 | −1.7 | −1.6 |
| 36 | AA466852_cysteine_rich_protein_61 | 2 | lung, bladder | lung, pl/ut | −1.2 | −1.3 | 1.1 | 1.1 |
| 16 | 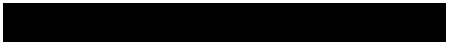 | 2 | bladder; mammary gland | none | −17 | −22 | −9.5 | −6.7 |
| 79 | 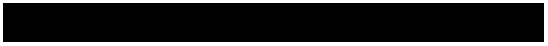 | 1 | bladder | none | −31 | −29 | −16 | −13 |
| 74 | 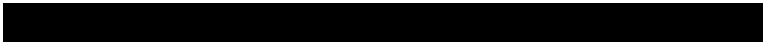 | 1 | bladder | none | −2.4 | −21 | −14 | −8.9 |
| 75 | 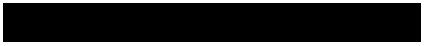 | 1 | bladder | none | −40 | −34 | −24 | −14 |
| 110 | 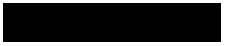 | 1 | brain | none | 1.2 | 1.4+ | 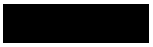 | 1.4 |
| 89 | 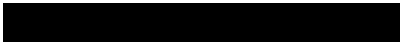 | 1 | brain | none | −1.1+ | −1.3 | −1.1+ | −1.1 |
| 102 | 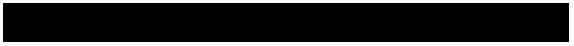 | 1 | brain | none | −1 | −1.2 | 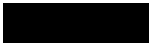 | 1.6 |
| 86 | 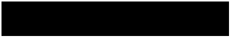 | 1 | brain | none | 1.3 | 1.2 | −1.5 | −1.5 |
| 103 | 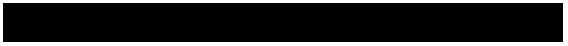 | 1 | brain | none | 1.1+ | [symbol] | [symbol] | 1.3 |
| 93 | 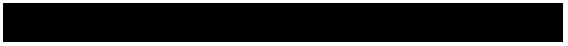 | 1 | brain | none | 1.1 | −1.2 | 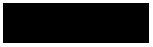 | −1 |
| 18 | AA270885_parvalbumin | 2 | colon; placenta/uterus | none | −12 | −11 | −5.7 | −4.1 |
| 110 | AA763276_small_protein,_X-linked | 1 | colon | none | −2.8 | −1.7 | −1 | 1.4 |
| 121 | AA671135_myosin_heavy_chain,_cardiac_muscle,_adult | 1 | colon | none | −1.1 | −9.7 | −9.9 | −5.8 |
| 126 | AA795264_ubiquitin-like_1 | 1 | colon | none | −6.5 | −6.5 | −5.6 | −3.1 |
| 127 | AI893944_forkhead_box_G1 | 1 | colon | none | −1.7 | −1 | 1.2 | 1.3 |
| 108 | 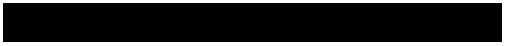 | 1 | colon | none | −5.5 | −6.5 | −7.9 | −4.7 |
| 146 | 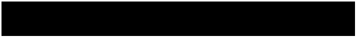 | 1 | colon | none | −12 | −13 | −7.8 | −4 |
| 153 | AA770902_actin,_alpha_1,_skeletal_muscle | 1 | intestien | none | −5.2 | −5.2 | −14 | −13 |
| 182 | AA821997_myoglobin | 1 | kidney | noen | −26 | −26 | −19 | −7.8 |
| 192 | 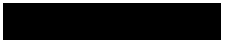 | 1 | kidney | none | −1.6+ | 1.1 | −1.3+ | 1.1 |
| 216 | AA712088_3-hydroxy-3-methylglutaryl- | 1 | liver | none | −3.4 | −3.1 | −3.7 | −2.3 |
| | Coenzyme_A_reductase | | | | | | | |
| 221 | 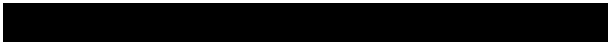 | 1 | liver | none | [symbol] | 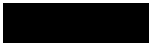 | −1.1 | −1 |
| 234 | 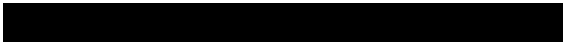 | 1 | liver | none | −1.8 | [symbol] | [symbol] | −1.4 |
| 231 | 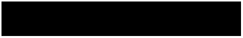 | 1 | liver | none | −1.5+ | −1.4+ | −1.4+ | −1.1 |
| 224 | 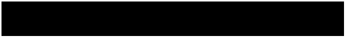 | 1 | liver | none | 1.6 | 1.5 | −1.2 | −1.3 |
| 35 | 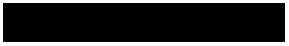 | 2 | lung, bladder | none | −45 | −53 | −1.1 | −1.2 |
| 247 | AA433639_heat_shock_protein_cognate_70,_testis | 1 | lung | none | −1 | −1.7 | −2.9 | −3.2 |
| 256 | AI046562_lactate_dehydrogenase_3,_C_chain,_sperm_specific | 1 | mammary gland | none | −5.6 | −6.4 | −4.4 | 2.8 |
| 259 | 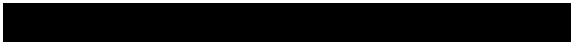 | 1 | mammary gland | none | 1.1 | 1.5 | −1.3 | −1.2 |
| 251 | 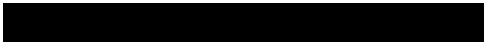 | 1 | mammary gland | none | −3.5 | −6.1 | −3.8 | −2.1 |
| 1 | 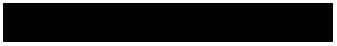 | 7 | pancreas; stomach; intestine; lung; bladder; placenta/uterus; mammary gland | none | −1 | −1.7 | 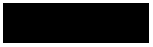 | 1.6 |
| 265 | 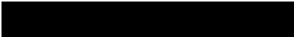 | 1 | pancreas | none | −1.9 | −1.9 | [symbol] | −3.2 |
| 270 | AA062401_TATA_box_binding_protein_(TBP)as- sociated_factor,_RNA_polymerase_II,_S,_150kD | 1 | pancreas | none | −2.4 | −2.9 | 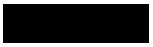 | −1.9 |
| 287 | AA600571_intracisternal_A_particles | 1 | spleen | none | 1.5 | −1.3 | −6 | −2 |
| 49 | AI550596_AI550596_stearoyl-Coenzyme_A_desaturase_2 | 2 | stomach; brain | none | −1.4 | −1.9 | 1.5 | 1.3 |
| 5 | 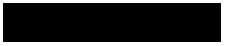 | 4 | stomach; brain; placenta/uterus; mammary gland | none | −1.2 | −1.4 | 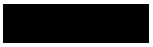 | 1.2 |
| 59 | AA591707_DNA_polymerase_epsilon,_subunit_2 | 2 | stomach; colon | none | 1.2 | 1.3 | 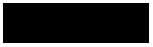 | 1.4 |
| 71 | AA544433_dihydroorotate_dehydrogenase | 2 | stomach; placenta/uterus | none | 1 | −1.2 | 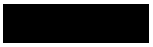 | 1.4 |
| 72 | AI037649_transition_protein_1 | 2 | stomach; placenta/uterus | none | −3 | −2.7 | −1 | −1.2 |
| 70 | AI553232_ribosomal_protein_S6 | 2 | stomach; placenta/uterus | none | 1.5 | 1.3 | 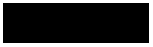 | 1.3 |
| 368 | 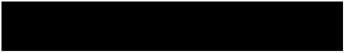 | 1 | stomach | none | 1.1 | −1 | 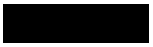 | 1.5 |
| 304 | 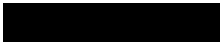 | 1 | stomach | none | 1.1 | −1 | 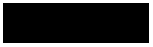 | 1.6 |
| 398 | 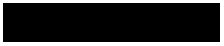 | 1 | stomach | none | 1.2 | 1.3 | 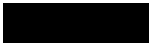 | [symbol] |
| 302 | 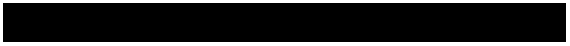 | 1 | stomach | none | 1.2 | 1.3 | 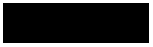 | 1.5 |
| 429 | 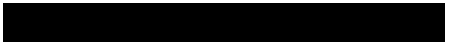 | 1 | stomach | none | 1.1 | −1.1+ | 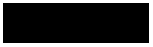 | 1.2 |
| 402 | 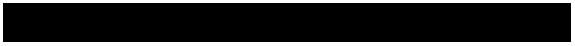 | 1 | stomach | none | −1.1 | −1.1 | 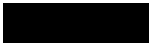 | 1.6 |
| 332 | 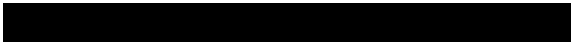 | 1 | stomach | none | −1.1 | −1.1+ | 1.6+ | 1.1 |
| 383 | A1643201_Mus_musculus_TOB3_mRNA,_complete_cds | 1 | stomach | none | −1.1 | −1.5 | −1.6 | −1.2 |
| 310 | AI549635_ESTs | 1 | stomach | none | −1.1 | −1.2 | 1.1 | −1 |
| 90 | 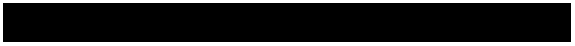 | 1 | brain | panc | −1.1 | −1.1 | 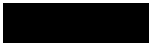 | 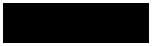 |
| 105 | 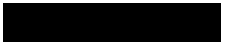 | 1 | brain | panc | 1.2 | −1.2* | 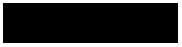 | 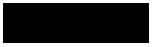 |
| 37 | 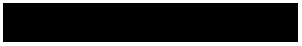 | 2 | lung, bladder | panc | −23 | −42 | 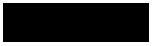 | 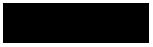 |
| 248 | AA388685_ESTs | 1 | lung | panc | 1.3 | −1.2 | 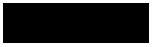 | 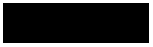 |
| 2 | AI549624_RIKEN_cDNA_061004E09_gene | 7 | pancreas; stomach; intestine; lung; bladder; placenta/uterus; mammary gland | panc | 1 | [symbol] | 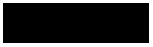 | 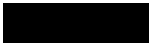 |
| 272 | 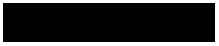 | 1 | pancreas | panc | 1.1 | −1.2 | 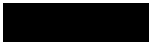 | 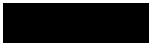 |
| 273 | 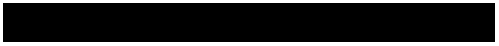 | 1 | pancreas | panc | 1.2 | −1.2 | 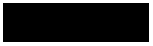 | 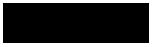 |
| 275 | 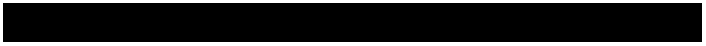 | 1 | pancreas | panc | 1.1 | −1.3 | 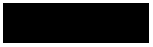 | 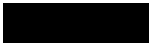 |
| 274 | 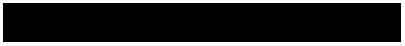 | 1 | pancreas panc | −1 | −1.4 | 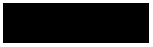 | 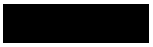 | |
| 39 | AI876997_carboxyl_ester_lipase | 2 | placenta/uterus; mammary gland | panc | −5.9 | −8.9 | 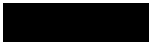 | 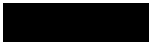 |
| 41 | 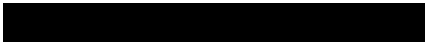 | 2 | spleen; brain | panc | [symbol] | −9.4 | 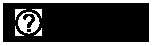 | 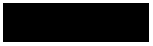 |
| 285 | AA982254_RIKEN_cDNA_2210010C04_gene | 1 | spleen | panc | −5.4 | −13 | 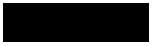 | 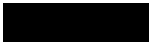 |
| 288 | AA674409_pancreatic_lipase_related_protein_1 | 1 | spleen | panc | −1.9 | −10 | 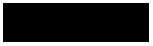 | 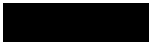 |
| 282 | AA675084_elastase_2 | 1 | spleen | panc | −5.8 | −13 | 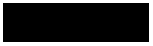 | 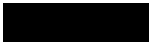 |
| 45 | AA155097_ESTs | 2 | stomach; bladder | panc | 1.2 | −1.1 | 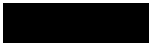 | 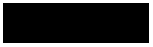 |
| 11 | AA153231_Public_domain_EST | 3 | stomach; bladder; placenta/uterus | panc | −1 | −1.3 | 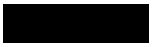 | 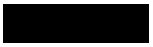 |
| 10 | AA561920_RalBPI_associated_Eps_domain_containing_protein | 3 | stomach; bladder; placenta/uterus | panc | 1.1 | −1.3 | 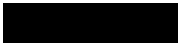 | 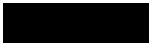 |
| 13 | AA940207_calcium/calmodulin- dependent_protein_kinase_kinase_1,_alpha | 3 | stomach; bladder; mammary gland | panc | 1.4 | −1.1* | 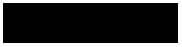 | 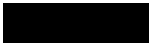 |
| 46 | AA469724_WW_domain_binding_protein_5 | 2 | stomach; bladder | panc | 1 | −1.3 | 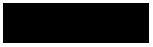 | 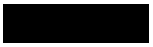 |
| 54 | 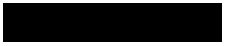 | 2 | stomach; brain | panc | 1 | −1.2 | 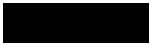 | 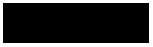 |
| 52 | 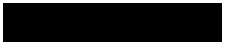 | 2 | stomach; brain | panc | −1.1 | −1.3 | 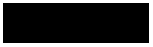 | 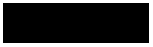 |
| 51 | 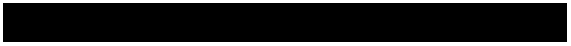 | 2 | stomach; brain | panc | 1.4 | −1 | 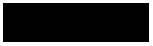 | 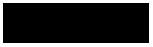 |
| 14 | 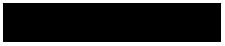 | 3 | stomach; intestine; lung | panc | 1 | −1.2 | 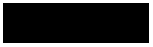 | 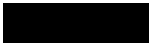 |
| 66 | 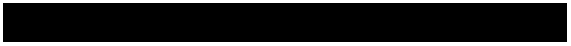 | 2 | stomach; lung | panc | 1.4 | −1.1 | 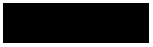 | 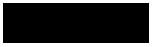 |
| 4 | 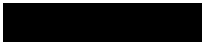 | 5 | stomach; lung; bladder; placenta/uterus; mammary gland | panc | 1.2 | −1.2 | 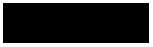 | 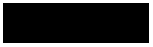 |
| 7 | 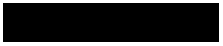 | 4 | stomach; lung; bladdr; placenta/uterus | panc | 1.1 | −1.4 | 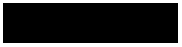 | 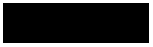 |
| 68 | 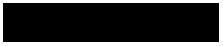 | 2 | stomach; mammary gland | panc | 1 | −1.6 | 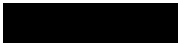 | 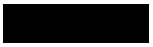 |
| 67 | 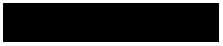 | 2 | stomach; mammary gland | panc | 1 | −1.3 | 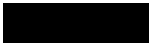 | 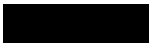 |
| 15 | 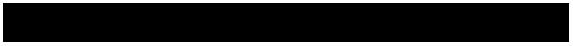 | 3 | stomach; placenta/uterus; mammary gland | panc | 1.3 | −1.2 | 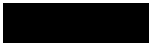 | 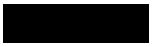 |
| 335 | 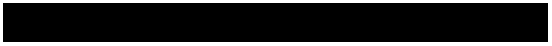 | 1 | stomach | panc | 1.1 | −1.1 | 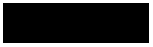 | 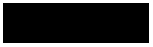 |
| 354 | 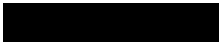 | 1 | stomach | panc | 1.1 | −1.1 | 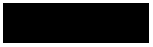 | 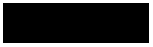 |
| 355 | 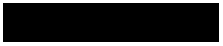 | 1 | stomach | panc | 1.1 | −1.1 | 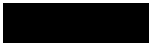 | 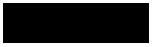 |
| 365 | 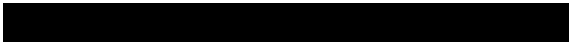 | 1 | stomach | panc | 1.1 | −1.1 | 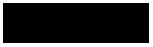 | 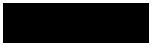 |
| 372 | 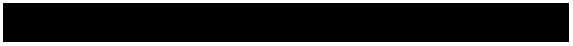 | 1 | stomach | panc | 1.2 | −1.1 | 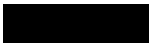 | 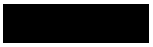 |
| 376 | 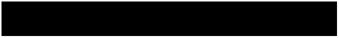 | 1 | stomach | panc | 1.3 | −1.1 | 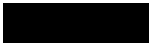 | 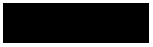 |
| 387 | AA671409_corel_UDP-galactose-N-acetylgalactosamine- alpha-R_beta_1,3-galactosyltransferase | 1 | stomach | panc | 1.2 | −1.2 | 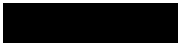 | 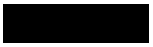 |
| 389 | 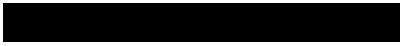 | 1 | stomach | panc | 1 | −1.1 | 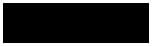 | 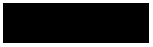 |
| 391 | 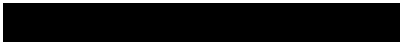 | 1 | stomach | panc | 1.2 | −1.1 | 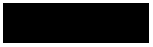 | 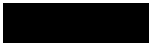 |
| 399 | 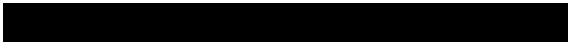 | 1 | stomach | panc | 1.2 | −1.1 | 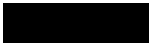 | 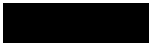 |
| 405 | 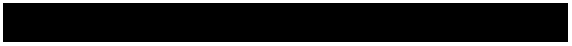 | 1 | stomach | panc | 1.1 | −1.1 | 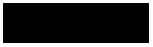 | 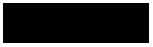 |
| 407 | 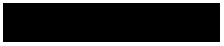 | 1 | stomach | panc | 1.2 | −1.1 | 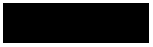 | 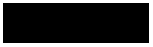 |
| 418 | 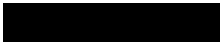 | 1 | stomach | panc | 1.2 | −1.1 | 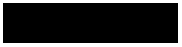 | 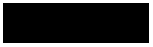 |
| 420 | AI463731_ESTs | 1 | stomach | panc | 1.1 | −1.1 | 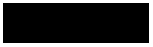 | 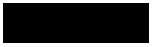 |
| 422 | AA273401_Public_domain_EST | 1 | stomach | panc | 1.1 | −1.3* | 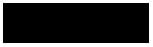 | 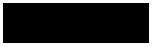 |
| 374 | AI549656_RIKEN_cDNA_3632413F13_gene | 1 | stomach | panc | 1.2 | 1 | 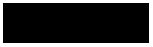 | 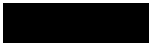 |
| 303 | AA688667_ESTs,_Weakly_similar_to_Inv_[M.musculus] | 1 | stomach | panc | 1.5 | 1.2 | 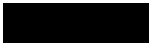 | 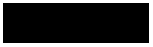 |
| 388 | AA269742_RIKEN_cDNA_1110065A22_gene | 1 | stomach | panc | 1.1 | −1 | 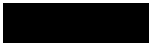 | 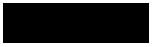 |
| 403 | AA220774_ESTs | 1 | stomach | panc | 1 | −1.2 | 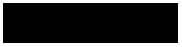 | 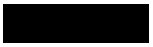 |
| 390 | AI587986_ESTs | 1 | stomach | panc | 1.1 | 1 | 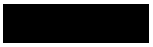 | 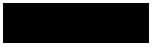 |
| 377 | AI645426_diphanous_homolog_3_(Drosophila) | 1 | stomach | panc | 1.1 | −1.1 | 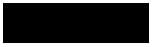 | 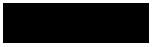 |
| 380 | AI596550_leukemia/lymphoma_related_factor | 1 | stomach | panc | 1.1 | −1.1 | 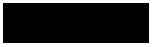 | 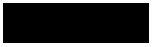 |
| 424 | AA412912_ESTs | 1 | stomach | panc | −1 | −1.2 | 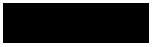 | 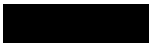 |
| 434 | AA244813_calbindin_2 | 1 | stomach | panc | −1 | −1.4 | 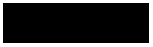 | 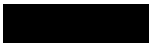 |
| 359 | AA116625_general_transcription_factor_IIH,_poly- peptide_2_(44_kDa_subunit) | 1 | stomach | panc | 1.3 | 1.1 | 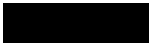 | 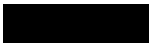 |
| 340 | AA823475_ESTs | 1 | stomach | panc | −1.1 | 1.1+* | 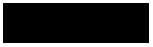 | 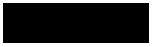 |
| 415 | AI604192_RIKEN_cDNA_4632433K11_gene | 1 | stomach | panc | 1.1 | −1.2 | 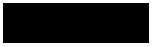 | 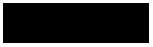 |
| 412 | AA268472_RIKEN_cDNA_2410003C20_gene | 1 | stomach | panc | 1 | −1 | 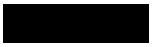 | 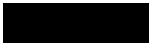 |
| 382 | AI464600_ESTs | 1 | stomach | panc | 1.1 | −1.1 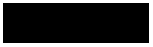 | 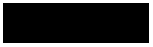 | |
| 314 | AA717080_ESTs | 1 | stomach | panc | 1.1 | 1 | 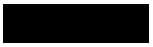 | 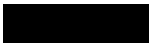 |
| 370 | AA516997_kinase_suppressor_of_ras | 1 | stomach | panc | −1.1 | −1.2 | 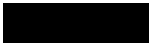 | 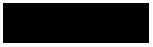 |
| 338 | AA591510_homeo_box_B1 | 1 | stomach | panc | −1.2 | −1.1 | 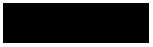 | 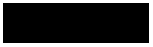 |
| 409 | AA798385_ESTs | 1 | stomach | panc | 1.3 | 1.4 | 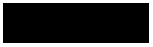 | 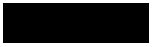 |
| 432 | AI592746_ESTs | 1 | stomach | panc | 1.1 | 1.2 | 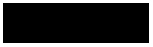 | 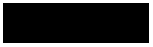 |
| 312 | AA646049_interleukin_13 | 1 | stomach | panc | 1.1 | −1.1 | 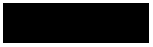 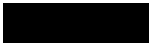 | |
| 331 | AA739464_killer_cell_lectin- like_receptor_subfamily_A,_member_9 | 1 | stomach | panc | 1.2 | −1.1 | 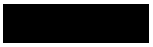 | 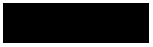 |
| 334 | AI594144_DNA_segment,_Chr_3,_Brigham_&_Women's_Gene- tics_0878_expressed | 1 | stomach | panc | 1.1 | −1 | 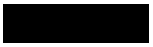 | 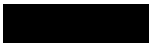 |
| 386 | AI627071_RIKEN_cDNA_2310058A11_gene | 1 | stomach | panc | 1.1 | −1.2 | 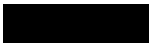 | 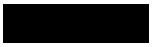 |
| 419 | AI551677_ESTs | 1 | stomach | panc | [symbol] | −1.3 | 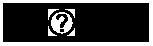 | 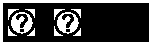 |
| 319 | AA058211_ESTs | 1 | stomach | panc | 1.1 | −1.2 | 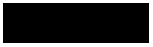 | 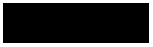 |
| 342 | AA615213_ESTs,_Moderately_similar_to_unnamed_pro- tein_product_[H.sapiens] | 1 | stomach | panc | −4.1 | −1 | 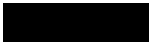 | 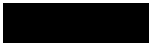 |
| 313 | AA691755_RIKEN_cDNA_1110068E08_gene | 1 | stomach | panc | 1.3 | 1 | 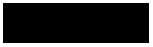 | 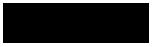 |
| 406 | AA290276_ESTs | 1 | stomach | panc | 1.4 | −1.1 | 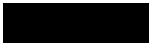 | 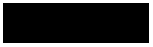 |
| 426 | 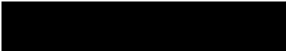 | 1 | stomach | panc | −1 | −1.4+ | 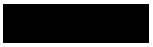 | 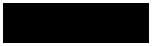 |
| 379 | AI594170_ESTs | 1 | stomach | panc | 1.1 | −1 | 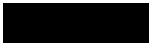 | 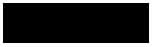 |
| 316 | AA067264_kenectin_1 | 1 | stomach | panc | −1.1 | 1.1 | 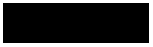 | 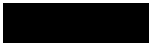 |
| 392 | 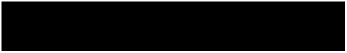 | 1 | stomach | panc | 1.3 | −1 | 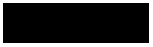 | 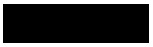 |
| 343 | AA608457_DNA_polymerase_epsilon | 1 | stomach | panc | 1.3 | 1.1 | 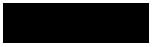 | 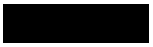 |
| 306 | AA596430_galactose-phosphate_uridyl_transferase | 1 | stomach | panc | 1.1 | −1.2 | 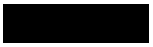 | 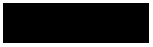 |
| 326 | AI789680_ESTs | 1 | stomach | panc | 1.1 | 1.1+ | 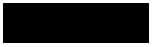 | 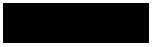 |
| 346 | AI607043_RIKEN_cDNA_2400007G07_gene | 1 | stomach | panc | −1 | −1.1 | 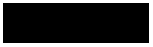 | 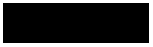 |
| 361 | AW210270_ESTs,_Weakly_similar_to_T17254_hypo- thetical_protein_DKFZp586O1022.1_[H.sapiens] | 1 | stomach | panc | 1.4 | 1.2 | 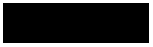 | 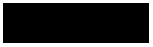 |
| 332 | AA266331_ESTs | 1 | stomach | panc | 1.2 | 1.1+ | 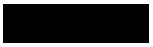 | 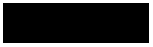 |
| 417 | AA543950_RIKEN_cDNA_2900073G15_gene | 1 | stomach | panc | −1 | −1.1 | 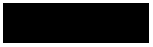 | 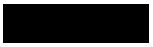 |
| 362 | AI390609_ESTs | 1 | stomach | panc | 1.2 | −1 | 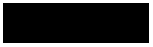 | 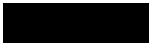 |
| 400 | AA517691_ESTs | 1 | stomach | panc | 1.2 | −1.1 | 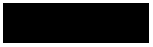 | 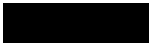 |
| 433 | AA638805_Public_domain_EST | 1 | stomach | panc | 1 | −1.1 | 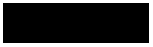 | 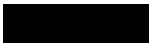 |
| 301 | AA709576_ESTs | 1 | stomach | panc | 1.1 | 1 | 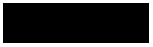 | 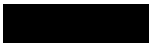 |
| 315 | AA709668_ESTs | 1 | stomach | panc | 1.2 | 1 | 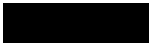 | 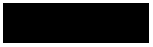 |
| 296 | AI614679_ESTs | 1 | stomach | panc | −1 | −1.2 | 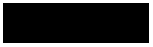 | 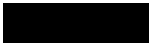 |
| 367 | AI450469_ESTs | 1 | stomach | panc | 1.1 | −1.1 | 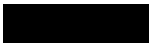 | 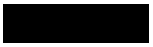 |
| 328 | AA616077_ESTs | 1 | stomach | panc | 1.5 | 1.2 | 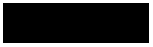 | 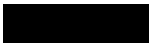 |
| 350 | AA268913_flavin_containing_monooxygenase_5 | 1 | stomach | panc | [symbol] | 1 | 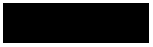 | 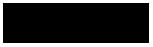 |
| 351 | AA210560_Mus_musculus,_Similar_to_RIKEN_cDNA_1 500041N16_gene,_clone_MGC:12066,_mRNA,_complete_cds | 1 | stomach | panc | 1.1 | −1 | 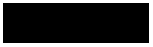 | 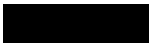 |
| 371 | AI607675_Mus_musculus,_clone_MGC:7865,_mRNA_com- plete_cds | 1 | stomach | panc | 1.2 | −1.1* | 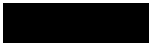 | 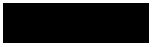 |
| 394 | AA265095_ESTs | 1 | stomach | panc | −1 | −1.2 | 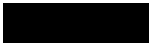 | 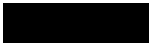 |
| 404 | AA727914_ESTs | 1 | stomach | panc | 1.3 | −1.2 | 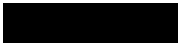 | 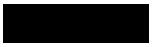 |
| 423 | AA204045_Public_domain_EST | 1 | stomach | panc | 1.1 | −1.3 | 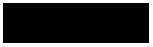 | 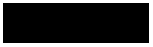 |
| 435 | AA623587_ESTs | 1 | stomach | panc | −1.1 | −1.1 | 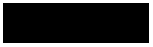 | 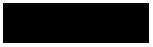 |
| 444 | AA259281_LIM_homeobox_protein_2 | 1 | stomach | panc | 1.3 | −1.1 | 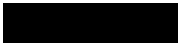 | 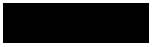 |
| 324 | AA822679_homatopoietic_cell_signal_transducer | 1 | stomach | panc | 1.2 | [symbol] | 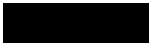 | 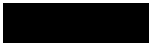 |
| 410 | AA473386_ESTs | 1 | stomach | panc | −1 | −1.2 | 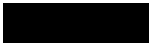 | 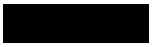 |
| 318 | AA259871_hypothetical_protein,_MNCb-4414 | 1 | stomach | panc | −1 | 1 | 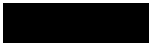 | 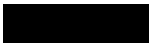 |
| 425 | AA474374_RIKEN_cDNA_5730408C10_gene | 1 | stomach | panc | 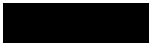 | 1.1 | 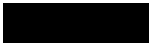 | 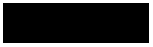 |
| 414 | AA230591_myosin,_heavy_poly- peptide_3,_skeletal_muscle_embryonic | 1 | stomach | panc | −1 | −1.4 | 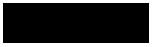 | 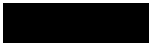 |
| 427 | AA607267_Public_domain_EST | 1 | stomach | panc | 1 | −1.4 | 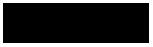 | 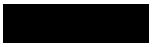 |
| 369 | AA469648_growth_factor,_erv1_(S._cerevisiae)- like_(augumenter_of_liver_regeneration) | 1 | stomach | panc | −1.1 | −1.5 | 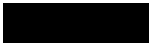 | 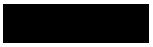 |
| 323 | AA060731_nuclear_factor,_interleukin_3,_regulated | 1 | stomach | panc | 1 | −1.3 | 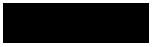 | 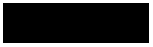 |
| 347 | AA638198_gap_junction_membrane_channel_protein_beta_3 | 1 | stomach | panc | −1.2 | −1.4 | 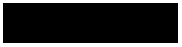 | 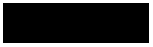 |
| 393 | AI530661_RIKEN_cDNA_2810426N06_gene | 1 | stomach | panc | 1.6 | 1.1 | 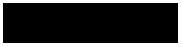 | 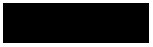 |
| 358 | AI466825_RIKEn_cDNA_A330103N21_gene | 1 | stomach | panc | −1 | −1.4 | 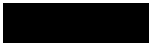 | 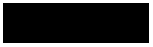 |
| 421 | AA162023_ESTs | 1 | stomach | panc | −1.1 | −1.4 | 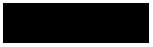 | 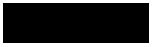 |
| 329 | AA611551_Mest-linked_imprinted_transcript_1 | 1 | stomach | panc, brain | −1 | −1.3+ | 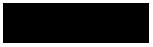 | 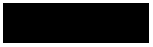 |
| 436 | AA277192_RIKEN_cDNA_2900075A18_gene | 1 | stomach | panc, brain | −1.4 | −1.5 | 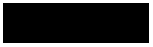 | 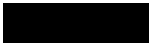 |
| 291 | AI117905_RIKEN_cDNA_6530406M24_gene | 1 | stomach | panc, brain | −1.2 | 1.3 | 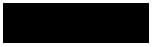 | 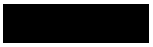 |
| 281 | AI550886_RIKEN_cDNA_1810010M01_gene | 1 | placenta/uterus | panc, col | −2.3 | −2.6 | 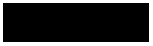 | 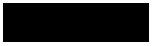 |

| | | 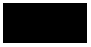 | | 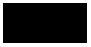 | | 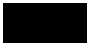 | | 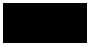 | |
|---|---|---|---|---|---|---|---|---|---|
| | | (−) | (+) | (−) | (+) | (−) | (+) | (−) | (+) |
| IDI | Description | PL-6B_BDE | PL-18B_BDE | PL-8B_BDE | PL-20B_BDE | PL-3B_BDE | PL-15B_BDE | PL-5B_BDE | PL-17B_BDE |
| 244 | AA726092_RIKEN_cDNA_4933419D20_gene | 1.2 | −1.2 | −1.2 | −1.3 | −1.1 | −1.7 | 1.1 | −1.5+ |
| 289 | AA222208_surfactant_associated_protein_C | −16 | −12 | −11 | −15 | −15 | −18 | −72 | −15 |
| 366 | AA710379_neuroplin | 1.5 | 1.1 | 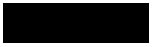 | −1.2 | 1.1 | −1.3 | −1.3 | 1.2+ |
| 344 | AI158775_protein_tyrosine_phosphatase,_receptor- type,_M | −1.2 | 1 | 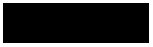 | 1 | 1.2 | 1 | −1.3 | −1.4+ |
| 204 | AA929863_ESTs,_similar_to_T46271_hypo- thetical_protein_DKFZp564P1263.1_[H.sapiens] | 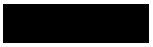 | 1.2 | −1.2 | −1.3 | −1.2 | −1.2 | −1.3 | −1.5 |
| 157 | AI892437_transglutaminase_2,_C_polypeptide | −1.3 | −1.4 | −1.9 | −2.4 | −1.1 | −2.6 | −1.4 | −1.1 |
| 203 | W18463_thioether_S-methyltransferase | 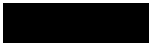 | 1.1 | −5.2 | −4.1 | −9 | −5.6 | −6.8 | −4.1 |
| 200 | AI117310_flavin_containing_monooxygenase_1 | 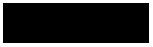 | 1.5 | −3.7 | −2.2 | −4.9 | −3.5 | −4.8 | −4 |
| 135 | AA670943_phosphatidic_acid_phosphotase_2a | −1.4 | −1.9 | −1.4 | −1.6 | 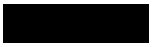 | 1.2 | 1.3 | −1.5 |
| 172 | AI552087_IQ_motif_containing_GTPase_activating_protein_1 | −2 | −2.5 | −1.1 | −1.2 | 1.1 | −1.8 | 1.3 | 1.1 |
| 36 | AA466852_cysteine_rich_protein_61 | −1.1 | 1 | 1.1 | 1.1 | −1.1 | −1.1 | −1.1 | [symbol] |
| 16 | 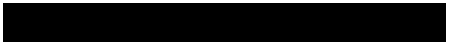 | −10 | −8.5 | −5.9 | −10 | −12 | −11 | −11 | −7.7 |
| 79 | 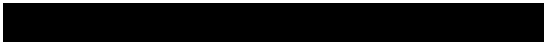 | −16 | −14 | −15 | −16 | −19 | −20 | −15 | −16 |
| 74 | 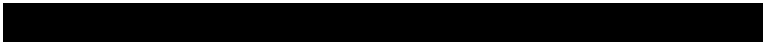 | −11 | −11 | −15 | −11 | −16 | −13 | −9.1 | −14 |
| 75 | 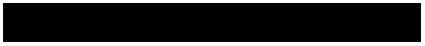 | −18 | −18 | −22 | −19 | −26 | −23 | −13 | −19 |
| 110 | 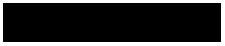 | 1.4 | [symbol] | 1.4 | 1.1 | 1.4 | 1.2 | 1.1 | 1.4+* |
| 89 | 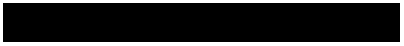 | 1+ | −1.4 | −1.3 | −1.5 | −1.6+ | −1.7+ | −1.5 | −1.3+ |
| 102 | 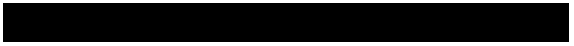 | −1 | 1.1 | 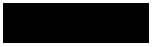 | −1.1 | 1.1 | −1.1 | −1.1 | −1+ |
| 86 | 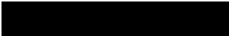 | −1.6 | −1.1 | −1.5 | [symbol] | −1.2 | −1.3 | −1.5 | −1.4* |
| 103 | 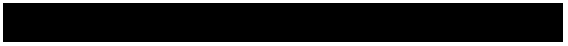 | [symbol] | 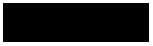 | 1.3 | 1.1 | 1.5 | 1.3+ | 1.1 | 1.1+ |
| 93 | 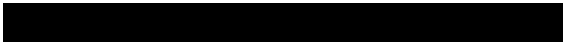 | −1.6 | −1.5 | −1.1 | −1.3 | −1.1 | −1 | −1.3 | −1.3+* |
| 18 | AA270885_parvalbumin | −6.8 | −4.7 | −4.4 | −6.2 | −5.3 | −7.1 | −6.1 | −12 |
| 110 | AA763276_small_protein,_X-linked | 1.4 | 1.3 | 1.1 | −1.4 | 1 | −1.2 | 1.5 | −1.2 |
| 121 | AA671135_myosin_heavy_chain,_cardiac_muscle,_adult | −5.5 | −7.2 | −6.2 | −7.2 | −7.6 | −8.3 | −4.1 | −8.5 |
| 126 | AA795264_ubiquitin-like_1 | −3.1 | −3.8 | −2.8 | −3.1 | −3.9 | −4.2 | −2 | −3.8 |
| 127 | AI893944_forkhead_box_G1 | 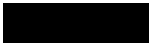 | 1.3 | 1.3 | −1.1 | 1.6 | 1.1 | 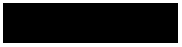 | 1.4* |
| 108 | 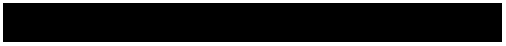 | −5 | −5.1 | −5.1 | −5.6 | −7.1 | −5.9 | −3.5 | −7.7 |
| 146 | 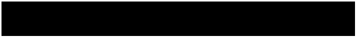 | −7.4 | −5.5 | −3.7 | −6.4 | −5.3 | −8 | −5.5 | −13 |
| 153 | AA770902_actin,_alpha_1,_skeletal_muscle | −13 | −12 | −2.3 | −2 | −1.9 | −4.3 | −2 | −2.1 |
| 182 | AA821997_myoglobin | −11 | −11 | −8.9 | −9.4 | −1.5 | −1.5 | −9 | −7.7 |
| 192 | 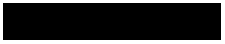 | −1.2 | 1 | 1+ | −1.1 | −1.2 | 1.2 | −1.1 | [symbol] |
| 216 | AA712088_3-hydroxy-3-methylglutaryl- | 1.1 | −1.8 | −2.2 | −1.7 | −2.6 | −1.7 | −1.9 | 1.1+ |
| | Coenzyme_A_reductase | | | | | | | | |
| 221 | 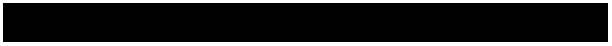 | 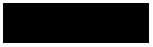 | 1.4 | −1.1 | −1.2 | 1 | 1.2 | −1.1 | −1.1+ |
| 234 | 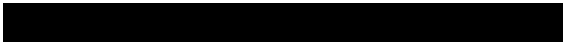 | 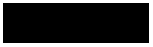 | 1.2 | −1.5 | −2 | 1.5 | 1.1 | −1.3 | −1.2+ |
| 231 | 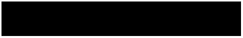 | 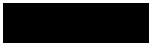 | 1.4 | −1.4+ | −1.3 | −1.2+ | −1.7+ | −1.1+ | −1.2+ |
| 224 | 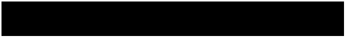 | 1 | −2.1* | 1 | −1.5 | −1 | −1.1 | 1.3 | −1.1 |
| 35 | 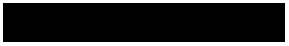 | −34 | −31 | −27 | −18 | −38 | −20 | −18 | −15 |
| 247 | AA433639_heat_shock_protein_cognate_70,_testis | −1.2 | −1.1 | −1.1 | −1.4 | −1.8 | −2.1 | −1.8 | −1.1 |
| 256 | AI046562_lactate_dehydrogenase_3,_C_chain,_sperm_specific | −3.6 | −2.8 | −2 | −1 | −2.7 | −3.4 | [symbol] | [symbol] |
| 259 | 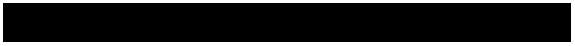 | −1 | −1.1 | −1.3 | −1.2 | −1.2 | −1.1 | −1.1 | −1.3+ |
| 251 | 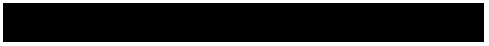 | −3.8 | −2.5 | −2.7 | −2.9 | −3.4 | −3.9 | −3 | −3.1 |
| 1 | 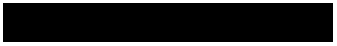 | −1.3 | 1.1 | 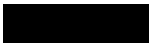 | −1.5 | 1.5 | −1.3 | −1.2 | −1.6+* |
| 265 | 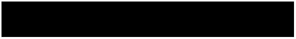 | −2.4 | −2.6 | −2.8 | −1.5 | −2.3 | −2.4 | −2.7 | −2.2 |
| 270 | AA062401_TATA_box_binding_protein_(TBP) | 1.2 | 1.1 | −1.2 | 1.2 | −6.6 | −3.6 | −4.6 | [symbol] |
| | associated_factor,_RNA_polymerase_II,_S,_150kD | | | | | | | | |
| 287 | AA600571_intracisternal_A_particles | −3.1 | −3 | −3.5 | −3.8 | −2.5 | −1.8 | [symbol] | −1.4 |
| 49 | AI550596_AI550596_stearoyl-Coenzyme_A_desaturase_2 | −1.7 | −1.3 | 1.4 | −1.5 | −1.1 | −1.6 | −1.2 | 1.2 |
| 5 | 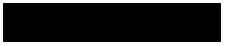 | −1.8 | −1.2 | 1.6 | −1.6 | 1.2 | −1.4 | −1.2 | −1.4+ |
| 59 | AA591707_DNA_polymerase_epsilon,_subunit_2 | 1.5 | 1.4* | 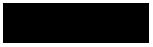 | −1 | 1.5 | 1 | 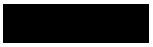 | 1.1 |
| 71 | AA544433_dihydroorotate_dehydrogenase | −1.1 | −1.1 | 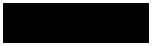 | −1.4 | 1.3 | −1.3 | −1.2 | −1.5+ |
| 72 | AI037649_transition_protein_1 | −1.9 | −1.6 | 1.2 | −1.8 | −1.5 | −2 | −1.9 | −1.5 |
| 70 | AI553232_ribosomal_protein_S6 | 1.1 | 1.1 | 1.3 | −1.6* | 1.2 | −1 | −1.2 | [symbol] |
| 368 | 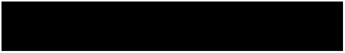 | 1 | 1.1 | 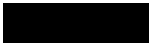 | 1 | 1.3 | 1.3 | −1.1 | −1.2+* |
| 304 | 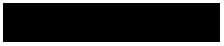 | 1.2+ | 1.2 | 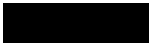 | −1.1 | 1.4 | 1.2 | −1.1 | 1.2 |
| 398 | 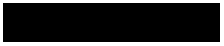 | 1.1 | 1.2 | 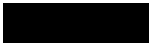 | −1.2 | 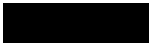 | 1.3 | 1.1 | [symbol] |
| 302 | 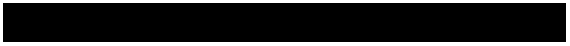 | 1.1+ | 1.3 | 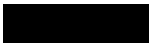 | −1 | 1.4 | 1.2 | 1 | 1.2+ |
| 429 | 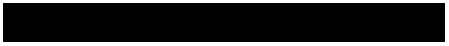 | −1.3 | 1.1 | 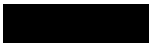 | −1.3 | 1.4 | −1.1 | −1.1 | −1.2+ |
| 402 | 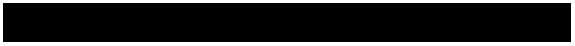 | −1.1 | 1.3 | 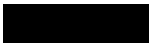 | −1.2 | 1.6 | −1.1 | 1.1 | −1.1 |
| 332 | 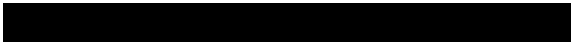 | 1+ | 1.3 | 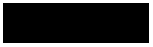 | −1.2 | 1.4 | −1.2 | 1 | 1.1+ |
| 383 | A1643201_Mus_musculus_TOB3_mRNA,_complete_cds | −1.1 | −1.3 | 1.3 | −1.6 | 1.2 | −1.2 | −1.4 | −1.6+ |
| 310 | AI549635_ESTs | −1.5 | −1.3 | 1.3 | −1.5 | −1.2 | −1.4 | −1.1* | −2.3+ |
| 90 | 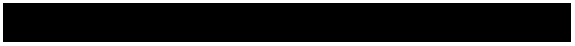 | −1 | 1.4 | 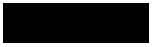 | 1 | 1.4 | −1 | 1 | −1.6+ |
| 105 | 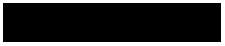 | 1.2 | 1.2 | 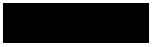 | −1 | 1.3 | 1.1 | 1.2* | |
| 37 | 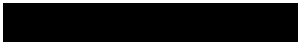 | −15 | −36 | −9 | −6.7 | −20 | −704 | −8.8 | −3.7 |
| 248 | AA388685_ESTs | 1.2 | 1.1 | 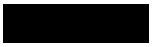 | −1* | 1.4 | 1 | −1 | 1.1+ |
| 2 | AI549624_RIKEN_cDNA_061004E09_gene | −1.3 | −1 | 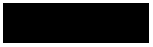 | −1.6 | 1.4 | −1.4 | −1.3 | −1.3+ |
| 272 | 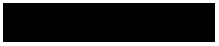 | 1.1 | 1.1 | 1.5 | 1.1 | 1.1 | −1 | −1 | −1** |
| 273 | 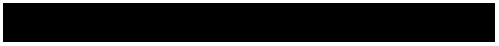 | 1.2 | 1.1 | 1.5 | 1.1 | 1.2 | 1 | −1 | 11 |
| 275 | 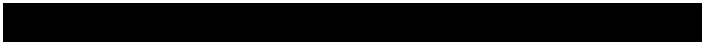 | 1.1 | 1.1 | 1.4 | 1 | 1.2 | −1.1 | −1 | −1* |
| 274 | 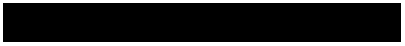 | 1.1 | 1.1 | 1.4 | −1.1 | 1.1 | −1.1 | −1.1 | −1.2** |
| 39 | AI876997_carboxyl_ester_lipase | −13 | −9.4 | −2.3 | 1 | −11 | −1.3 | −10 | −3.5 |
| 41 | 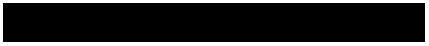 | −36 | −36 | −2 | 1.4 | −30 | 1.1 | −24 | −6 |
| 285 | AA982254_RIKEN_cDNA_2210010C04_gene | −18 | −14 | −2.8 | −1.4 | −15 | −1.5 | −10 | −8 |
| 288 | AA674409_pancreatic_lipase_related_protein_1 | −12 | −9.1 | −1.9 | −1.1 | −8.1 | −1.4 | −8.2 | −8.1 |
| 282 | AA675084_elastase_2 | −27 | −23 | −2.5 | 1.1 | −26 | −1.2 | −16 | −7.6 |
| 45 | AA155097_ESTs | 1.3 | 1.2 | 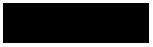 | −1 | 1.3 | 1.1 | −1 | 1.2 |
| 11 | AA153231_Public_domain_EST | 1.1 | 1 | 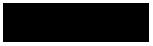 | −1.2 | 1.5 | −1.1 | 1.3** | 1.2 |
| 10 | AA561920_RalBPI_associated_Eps_domain_containing_protein | 1.1 | 1.2 | 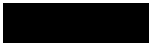 | −1.3 | 1.5 | −1 | −1 | 1.1 |
| 13 | AA940207_calcium/calmodulin- dependent_protein_kinase_kinase_1,_alpha | 1.2 | 1.1 | 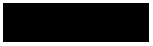 | −1.1* | 1.2 | 1 | −1.2 | 1.1 |
| 46 | AA469724_WW_domain_binding_protein_5 | −1.1 | 1 | 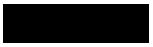 | −1.1 | 1.2 | −1.2 | −1.1 | −1.2 |
| 54 | 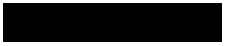 | 1.1 | 1.4 | 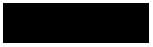 | 1 | 1.6 | 1 | 1 | 1.5 |
| 52 | 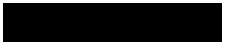 | 1 | 1.2 | 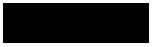 | −1.1 | 1.3 | −1.2 | −1.2 | −1.1+ |
| 51 | 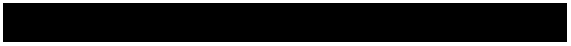 | 1.2 | 1.2 | 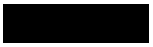 | −1.1 | 1.5 | 1.1 | −1.1 | 1.1 |
| 14 | 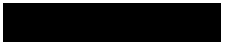 | 1 | 1.4 | 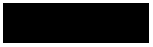 | −1.1 | 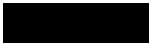 | −1 | 1.1 | 1.4 |
| 66 | 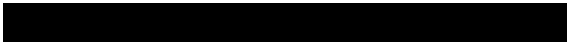 | 1.2 | 1.2 | 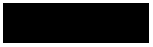 | −1.1 | 1.4 | 1.1 | −1.1 | 1.2 |
| 4 | 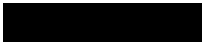 | 1.1 | 1.1 | 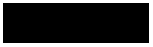 | −1.2* | 1 .3* | −1.1 | −1.3 | 1.2+ |
| 7 | 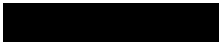 | −1.1 | −1.1 | 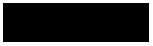 | −1.3 | 1.3 | −1.2 | −1.5 | −1.2+* |
| 68 | 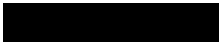 | 1.1 | 1.2 | 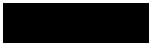 | −1.2 | 1.3 | −1.2 | [symbol] | −1.4+* |
| 67 | 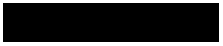 | −1 | 1.2 | 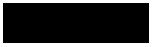 | −1.3 | 1.5 | −1.1 | −1.1 | −1.1+ |
| 15 | 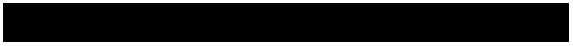 | 1.1 | 1.1 | 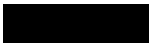 | −1.2 | 1.4* | 1 | −1.2 | 1.1 |
| 335 | 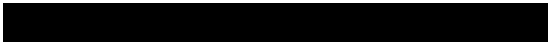 | 1.2 | 1.3 | 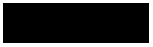 | 1 | 1.5 | 1.1 | 1.1 | −1+* |
| 354 | 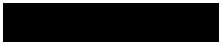 | 1.3 | 1.3 | 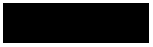 | 1 | 1.4 | 1.2 | 1.2 | 1.3+* |
| 355 | 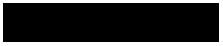 | [symbol] | [symbol] | 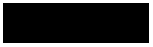 | −1.1 | 1.4 | 1.2 | −1 | [symbol] |
| 365 | 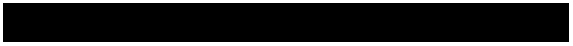 | 1 | 1.2 | 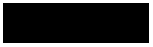 | −1.1 | 1.5 | −1 | 1.1 | −1.2+* |
| 372 | 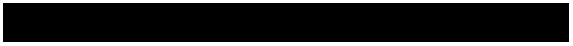 | 1.1 | 1.3 | 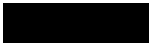 | −1.1 | 1.4 | 1.4 | 1.1 | 1* |
| 376 | 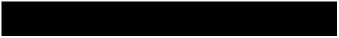 | 1.3 | 1.4 | 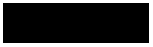 | 1.6 | 1 | 1.3 | 1.1+* | |
| 387 | AA671409_corel_UDP-galactose-N-acetylgalactosamine- alpha-R_beta_1,3-galactosyltransferase | 1.1 | 1.5 | 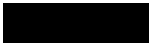 | 1 | 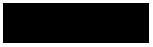 | 1.1 | 1.2 | [symbol] |
| 389 | 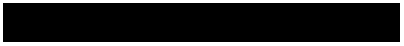 | 1.1 | 1.2 | 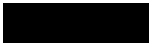 | −1.1 | 1.4 | −1 | 1.2 | −1.2+* |
| 391 | 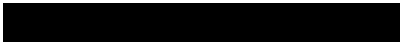 | 1.1 | 1.2 | 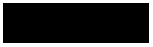 | −1.1 | 1.3* | 1 | −1 | 1.1+* |
| 399 | 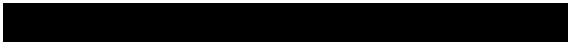 | 1.2 | 1.4 | 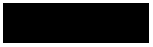 | −1 | 1.5 | 1 | 1 | 1.1* |
| 405 | 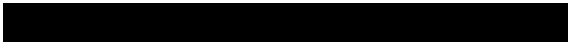 | 1.1 | 1.2 | 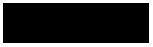 | −1.1 | 1.3 | 1 | −1 | 1.1 |
| 407 | 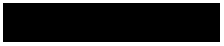 | 1.1 | 1.2 | 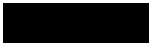 | −1.1 | 1.4 | 1 | −1 | 1 |
| 418 | 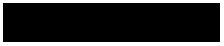 | 1.1 | 1.4 | 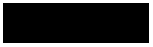 | −1.1 | 1.4 | 1.1 | 1 | 1.1+* |
| 420 | AI463731_ESTs | 1 | 1.4 | 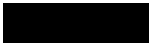 | −1.1* | 1.3 | 1 | −1 | 1 |
| 422 | AA273401_Public_domain_EST | 1.1 | 1.1 | 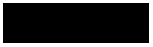 | −1.1 | 1.4* | −1 | 1 | 1 |
| 374 | AI549656_RIKEN_cDNA_3632413F13_gene | 1 | 1.3* | 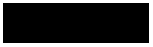 | −1.1 | 1.5 | 1.1 | 1 | 1.1 |
| 303 | AA688667_ESTs,_Weakly_similar_to_Inv_[M.musculus] | 1 | 1.4 | 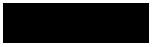 | 1.2 | 1.6 | 1.1 | 1 | −1.1+ |
| 388 | AA269742_RIKEN_cDNA_1110065A22_gene | 1.1 | 1.3 | 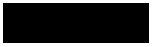 | 1.2 | 1.4 | 1.1 | [symbol] | −1.1+* |
| 403 | AA220774_ESTs | 1.3 | 1.3 | 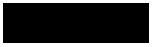 | −1 | 1.4 | 1.1 | −1.1 | −1 |
| 390 | AI587986_ESTs | 1.3 | 1.3 | 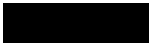 | 1.1 | 1.5 | 1.1 | 1.2 | −1+ |
| 377 | AI645426_diphanous_homolog_3_(Drosophila) | 1.1 | [symbol] | 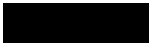 | −1 | 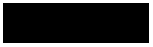 | [symbol] | [symbol] | 1.1+* |
| 380 | AI596550_leukemia/lymphoma_related_factor | 1.1 | 1.3 | 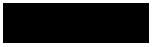 | 1 | [symbol] | 1.1 | 1.1 | [symbol] |
| 424 | AA412912_ESTs | 1.1 | 1.5 | 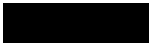 | −1 | 1.5 | −1 | 1.1 | 1.2+* |
| 434 | AA244813_calbindin_2 | −1 | 1.5 | 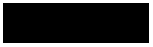 | 1.1 | 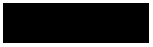 | 1 | −1 | 1.1+* |
| 359 | AA116625_general_transcription_factor_IIH,_poly- peptide_2_(44_kDa_subunit) | 1.3 | 1.2 | 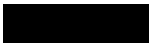 | −1.1 | 1.3 | 1.1 | −1 | 1.2 |
| 340 | AA823475_ESTs | 1.1 | 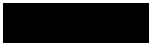 | 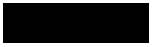 | 1.1 | 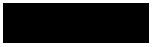 | 1.1 | 1.1 | [symbol] |
| 415 | AI604192_RIKEN_cDNA_4632433K11_gene | 1.1 | 1.2 | 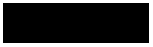 | −1 | 1.3 | 1.1 | −1.1 | −1.2+* |
| 412 | AA268472_RIKEN_cDNA_2410003C20_gene | 1.3 | 1.4 | 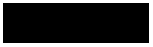 | 1 | 1.5 | 1.1 | 1.2 | [symbol] |
| 382 | AI464600_ESTs | 1 | 1.5 | 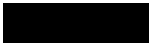 | 1.1 | 1.4 | 1.2 | 1.1 | 1.2+ |
| 314 | AA717080_ESTs | 1.3 | 1.4 | 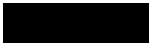 | 1.2 | 1.6 | 1.1 | 1.4 | −1.1+* |
| 370 | AA516997_kinase_suppressor_of_ras | 1 1.4 | 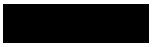 | 1 | 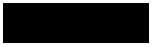 | 1.4 | 1.3 | 1.1+ | |
| 338 | AA591510_homeo_box_B1 | 1.1 | 1.5 | 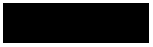 | 1 | 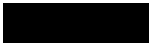 | 1 | 1.5 | −1.3+* |
| 409 | AA798385_ESTs | 1 | 1.1 | 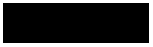 | 1 | 1.3 | 1.1 | −1.1 | −1+* |
| 432 | AI592746_ESTs | 1 | 1.1 | 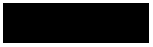 | −1.1 | 1.3 | 1 | −1 | −1+* |
| 312 | AA646049_interleukin_13 | 1.4 | 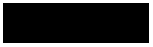 | 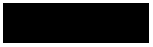 | 1.2 | 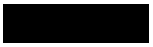 | 1.3 | 1.4 | 1.4+* |
| 331 | AA739464_killer_cell_lectin- like_receptor_subfamily_A,_member_9 | 1.2 | 1.3 | 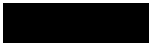 | 1 | 1.4 | 1 | −1.1 | −1** |
| 334 | AI594144_DNA_segment,_Chr_3,_Brigham_&_Women's_Gene- tics_0878_expressed | 1.3 | 1.5 | 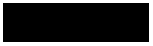 | 1.1 | 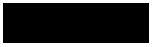 | 1.2 | 1.2 | 1.4 |
| 386 | AI627071_RIKEN_cDNA_2310058A11_gene | −1 | 1.3 | 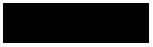 | −1 | −1.4 | −1.1 | −1.1 | 1.1+* |
| 419 | AI551677_ESTs | 1.1 [symbol] | 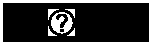 | 1 | 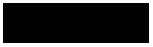 | [symbol] | [symbol] | [symbol] | |
| 319 | AA058211_ESTs | 1.3 | 1.3 | 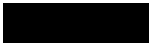 | −1.1 | 1.5 | 1.4 | 1.3 | 1.3 |
| 342 | AA615213_ESTs,_Moderately_similar_to_unnamed_pro- tein_product_[H.sapiens] | 1.2 | 1.4 | 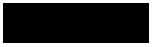 | 1.1 | 1.6 | 1.1 | 1.2 | −1.3+* |
| 313 | AA691755_RIKEN_cDNA_1110068E08_gene | 1.1+ | −1+** | 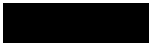 | 1.3 | 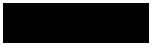 | 1.3 | 1 | 1.3+ |
| 406 | AA290276_ESTs | −1 | 1 | [symbol] | −1.2 | 1.2 | −1 | −1.1 | 1.1+* |
| 426 | 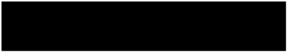 | 1.2 | 1.1 | 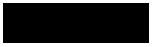 | −1.1 | 1.6 | −1.1 | −1 | 1.2+ |
| 379 | AI594170_ESTs | 1.3 | 1.6 | 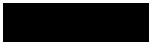 | −1 | 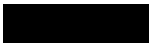 | 1.2 | 1.4 | 1.4 |
| 316 | AA067264_kenectin_1 | 1.1 | 1.5 | 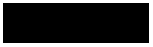 | 1 | [symbol] | −1 | 1.4 | −1.1+* |
| 392 | 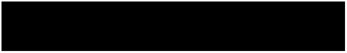 | 1.2 | −1 | 1.5 | −1.3 | 1 | −1.1 | −1.3 | 1.1+ |
| 343 | AA608457_DNA_polymerase_epsilon | −1 | 1.4 | 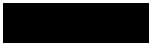 | −1.1 | 1.5 | −1.1 | 1.1 | −1+* |
| 306 | AA596430_galactose-phosphate_uridyl_transferase | 1.4 | 1.5 | 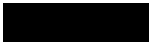 | 1 | 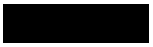 | 1.3 | 1.2 | 1.3+ |
| 326 | AI789680_ESTs | 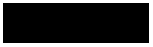 | 1.3 | 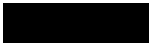 | 1.2 | [symbol] | 1.1 | 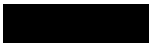 | [symbol] |
| 346 | AI607043_RIKEN_cDNA_2400007G07_gene | 1.4 | 1.6 | 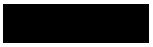 | 1 | 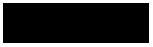 | 1.1 | 1.2 | 1.2+* |
| 361 | AW210270_ESTs,_Weakly_similar_to_T17254_hypo- thetical_protein_DKFZp586O1022.1_[H.sapiens] | 1.1 | 1.3 | 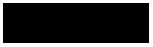 | −1.1 | 1.5 | 1.1 | 1 | 1.1 |
| 332 | AA266331_ESTs | 1.2 | 1.2 | 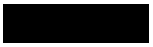 | 1.1 | 1.4 | 1.1 | 1.1 | 1.1+* |
| 417 | AA543950_RIKEN_cDNA_2900073G15_gene | 1.2 | 1.4 | 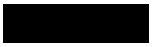 | −1.1 | [symbol] | −1 | 1 | 1.1 |
| 362 | AI390609_ESTs | 1.2 | 1.2 | 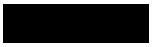 | −1 | 1.4 | −1 | 1.1 | 1+* |
| 400 | AA517691_ESTs | 1.1 | 1.3 | 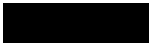 | −1.1 | 1.4* | 1.1 | 1 | 1.2 |
| 433 | AA638805_Public_domain_EST | 1.1 | 1.3 | 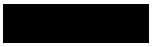 | −1.1 | 1.5 | 1 | −1.2 | −1.1+* |
| 301 | AA709576_ESTs | 1.1+ | 1.6 | 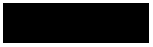 | 1.1 | 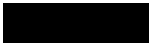 | 1.1 | 1.2 | 1.3+* |
| 315 | AA709668_ESTs | 1.4 | 1.6 | 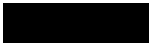 | 1.1 | 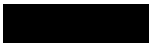 | 1.1 | 1.3 | 1.1+ |
| 296 | AI614679_ESTs | 1.2 | 1.3 | 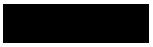 | −1 | 1.5 | −1.2 | 1.3 | 1.1 |
| 367 | AI450469_ESTs | 1.2 | 1.4 | 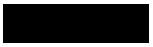 | −1.1 | 1.4 | 1.1 | 1.1 | 1.2 |
| 328 | AA616077_ESTs | 1 | 1.4 | 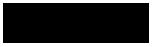 | 1.2 | 1.6 | 1.1 | −1 | 1.1+ |
| 350 | AA268913_flavin_containing_monooxygenase_5 | 1.1 | 1.2 | 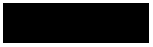 | −1 | 1.4 | 1.1 | 1.1 | [symbol] |
| 351 | AA210560_Mus_musculus,_Similar_to_RIKEN_cDNA_1 500041N16_gene,_clone_MGC:12066,_mRNA,_complete_cds | −1 | 1 | 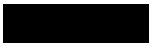 | −1.2 | 1.3 | 1 | −1.2 | [symbol] |
| 371 | AI607675_Mus_musculus,_clone_MGC:7865,_mRNA_com- plete_cds | 1.3 | 1.2 | 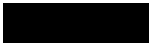 | −1.1 | 1.3 | 1.1 | −1.2* | 1.2 |
| 394 | AA265095_ESTs | 1.1 | 1 | 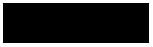 | −1 | 1.6 | 1.2 | −1 | 1.1+* |
| 404 | AA727914_ESTs | 1.1 | 1.2 | 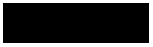 | −1 | 1.4 | −1.1 | 1.1 | 1.2+* |
| 423 | AA204045_Public_domain_EST | 1.1 | 1.1 | 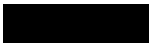 | −1.1 | 1.3 | −1.1 | 1 | 1.2 |
| 435 | AA623587_ESTs | 1 | 1.3 | 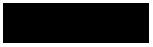 | −1.1 | 1.6 | 1 | −1 | 1.1+ |
| 444 | AA259281_LIM_homeobox_protein_2 | 1.1 | 1.2 | 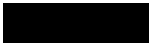 | −1 | 1.3 | 1.1 | −1.1 | 1.1* |
| 324 | AA822679_homatopoietic_cell_signal_transducer | −1 | 1.1 | 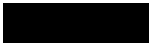 | −1.1 | 1.4 | 1.1 | −1.1 | 1.6+ |
| 410 | AA473386_ESTs | 1.2 | 1.5 | 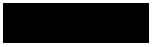 | 1.2 | 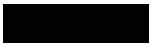 | 1.1 | 1.2 | 1.1+* |
| 318 | AA259871_hypothetical_protein,_MNCb-4414 | 1.2 | 1.3 | 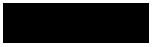 | 1 | 1.2 | 1 | −1.1 | −1+ |
| 425 | AA474374_RIKEN_cDNA_5730408C10_gene | 1.1 | 1 | 1.6 | −1.3 | 1.4 | 1.1 | −1.1 | −1 |
| 414 | AA230591_myosin,_heavy_poly- peptide_3,_skeletal_muscle_embryonic | 1 | 1.2 | 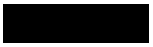 | −1.1* | 1.3 | −1 | 1 | −1.1+ |
| 427 | AA607267_Public_domain_EST | −1.1 | 1.4 | 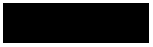 | −1.3 | 1.5 | −1 | 1.1 | 1.1+* |
| 369 | AA469648_growth_factor,_erv1_(S._cerevisiae)- like_(augumenter_of_liver_regeneration) | −1.1 | 1.3 | 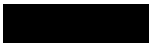 | −1.3 | 1.4 | −1.2 | −1.1 | −1.4+ |
| 323 | AA060731_nuclear_factor,_interleukin_3,_regulated | 1.1 | 1.2 | 1.6 | −1.2 | 1.2 | −1.1 | −1.2 | −1.4+ |
| 347 | AA638198_gap_junction_membrane_channel_protein_beta_3 | 1 | 1.1 | 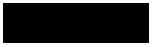 | −1.2 | 1.2 | −1 | −1.1 | −1.3 |
| 393 | AI530661_RIKEN_cDNA_2810426N06_gene | 1.2 | 1 | 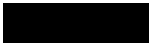 | −1.1 | 1.3 | 1.1 | −1.3 | 1.1* |
| 358 | AI466825_RIKEn_cDNA_A330103N21_gene | 1 | 1.2 | 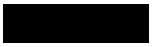 | −1.1 | 1.2 | −1.1 | −1 | −1.1 |
| 421 | AA162023_ESTs | 1 | 1.1 | 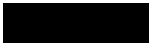 | −1.4 | 1.5 | −1.2 | 1 | −1.3+* |
| 329 | AA611551_Mest-linked_imprinted_transcript_1 | 1.2 | 1.2 | 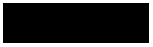 | 1.1 | [symbol] | −1.1 | 1 | 1.2+ |
| 436 | AA277192_RIKEN_cDNA_2900075A18_gene | [symbol] | 1.2 | 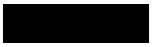 | −1.1 | 1.3 | −1.2 | −1.3 | −1.3+ |
| 291 | AI117905_RIKEN_cDNA_6530406M24_gene | 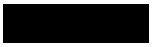 | 1.5 | 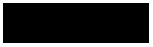 | −1.1 | 1.6 | 1.1 | 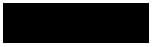 | 1.1+* |
| 281 | AI550886_RIKEN_cDNA_1810010M01_gene | −2.2 | −1.9 | 1.2 | −1.2 | 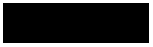 | −1.4 | 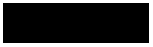 | 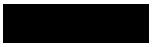 |
| | | 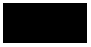 | | 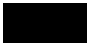 | | 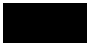 | | 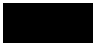 | |
| | | (−) | (+) | (−) | (+) | (−) | (+) | (−) | (+) |
| IDI | Description | PL-7B_BDE | PL-19B_BDE | PL-1B_BDE | PL-13B_BDE | PL-9B_BDE | PL-21B_BDE | PL-10B_BDE | PL-22B_BDE |
| 244 | AA726092_RIKEN_cDNA_4933419D20_gene | 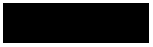 | 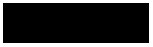 | −1.5 | −1.2 | −1 | −1.3 | 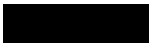 | 1.2 |
| 289 | AA222208_surfactant_associated_protein_C | 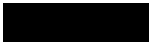 | 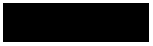 | −23 | −14 | −21 | −17 | −29 | −21 |
| 366 | AA710379_neuroplin | 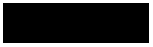 | 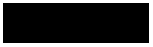 | 1 | 1.1 | 1.3 | 1.2 | 1.3 | 1.4 |
| 344 | AI158775_protein_tyrosine_phosphatase,_receptor- type,_M | 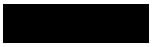 | 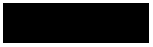 | 1.4 | 1.2 | 1.3 | 1.3 | 1.1 | 1 |
| 204 | AA929863_ESTs,_similar_to_T46271_hypo- thetical_protein_DKFZp564P1263.1_[H.sapiens] | 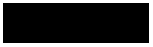 | 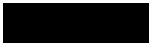 | −1.2 | 1.1 | 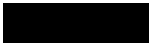 | 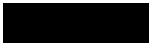 | −1.3 | −1.2 |
| 157 | AI892437_transglutaminase_2,_C_polypeptide | 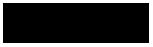 | 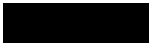 | −3.9 | −3.8 | 1.6 | 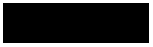 | 1.1 | −1 |
| 203 | W18463_thioether_S-methyltransferase | 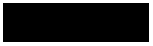 | 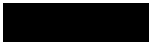 | −1.2 | −6.2 | −1.1 | 1.5 | 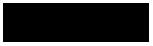 | 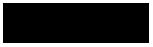 |
| 200 | AI117310_flavin_containing_monooxygenase_1 | 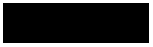 | 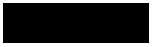 | −4.7 | −3.9 | −2.4 | −2.2 | 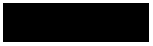 | 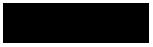 |
| 135 | AA670943_phosphatidic_acid_phosphotase_2a | 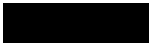 | 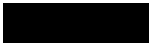 | −2 | −1.6 | 1.1 | 1 | 1.3 | 1.2 |
| 172 | AI552087_IQ_motif_containing_GTPase_activating_protein_1 | 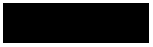 | 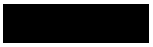 | −2.8 | −2.3 | 1.2 | 1.2 | −1.3 | −1.3 |
| 36 | AA466852_cysteine_rich_protein_61 | 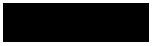 | 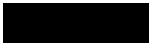 | −1.4 | −1.4 | 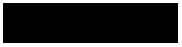 | 1.2 | 1.1 | 1.2 |
| 16 | 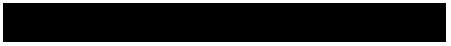 | −9.2 | −14 | −17 | −12 | −3.6 | −14 | −1.4 | −1.8 |
| 79 | 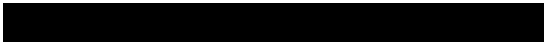 | −11 | [symbol] | −23 | −16 | −1.2 | −22 | −36 | −25 |
| 74 | 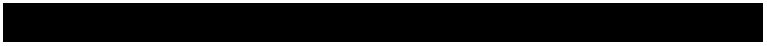 | −12 | −16 | −24 | −12 | −5.5 | −12 | −26 | −21 |
| 75 | 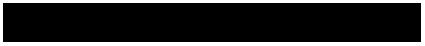 | −13 −24 | −36 | −18 | −4.5 | −29 | −56 | −35 | |
| 110 | 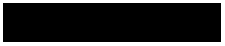 | 1.3 | 1.2 | 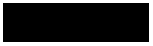 | 1.2 | 1.4 | 1.3 | 1.3 | −1 |
| 89 | 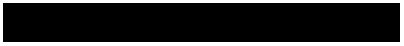 | −1.2 | −1.5 | 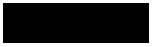 | 1.6 | −1.4 | −1.5 | −1.1 | −1.4 |
| 102 | 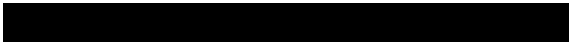 | −1 | −1.4 | 1.3 | [symbol] | 1.2 | −1.3 | 1.3 | −1.1 |
| 86 | 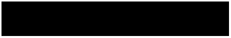 | 1.1 | −1.2 | 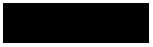 | 1.2 | −1.2 | −1.1 | −1.5 | −1.5 |
| 103 | 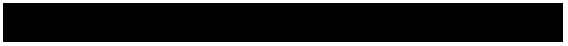 | 1.1 | 1.1+ | 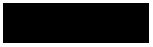 | 1.1 | 1.2 | 1.2+ | 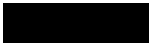 | 1.5 |
| 93 | 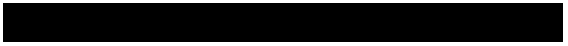 | −1.2 | −12. | 1 | −2.2 | −1.3 | −1.4 | 1 | −1.2 |
| 18 | AA270885_parvalbumin | −6.3 | −7.4 | −3.1 | −2.1 | −5 | −5.4 | −3.4 | −4.2 |
| 110 | AA763276_small_protein,_X-linked | −2.3 | 1.1 | −3.2 | 1.4 | −2.4 | −1.5 | −2.3 | −1.9 |
| 121 | AA671135_myosin_heavy_chain,_cardiac_muscle,_adult | −2.4 | −1.8 | −8 | −4.2 | −9 | −4.3 | −6.5 | −9.3 |
| 126 | AA795264_ubiquitin-like_1 | −3.6 | −2.3 | −4.8 | −2.7 | −4.1 | −3.1 | −4.7 | −5.1 |
| 127 | AI893944_forkhead_box_G1 | −1.5 | 1.1 | 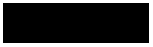 | 1.6 | [symbol] | −1 | −1.8 | −1.7 |
| 108 | 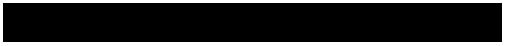 | −4.6 | −3.8 | −3.3 | −2.6 | −5.2 | −5.7 | −7.4 | −6.4 |
| 146 | 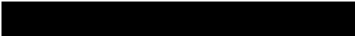 | −7.1 | −8.7 | −12 | −7.4 | −5.2 | −5.6 | −9.2 | −9.9 |
| 153 | AA770902_actin,_alpha_1,_skeletal_muscle | −3 | −3.2 | −7.7 | −5.2 | 1 | −1 | −8.1 | −8.9 |
| 182 | AA821997_myoglobin | −6 | −3.6 | −22 | −12 | −24 | −5.6 | −9.4 | −21 |
| 192 | 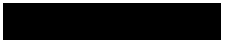 | 1.4+ | 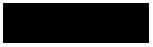 | −1.1 | −1.4 | −1.3 | −1.8 | 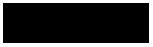 | 1.2 |
| 216 | AA712088_3-hydroxy-3-methylglutaryl- | −3.5 | −2.7 | −2.1 | −1.9 | −3 | −2.9 | −1.3 | −1.6 |
| | Coenzyme_A_reductase | | | | | | | | |
| 221 | 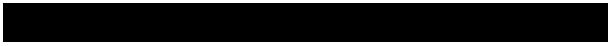 | 1.2 | 1.2 | 1.1 | −1.1 | 13 | 1.2 | −1.2 | 1 |
| 234 | 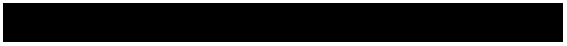 | −1.3 | −1.8 | −1.5+ | −1.8 | −1.7 | −1.8 | 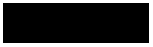 | 1.4 |
| 231 | 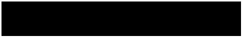 | 1.5 | 1.1 | −1.2+ | −1.1 | 1.4 | 1.1 | −1.1 | −1.1 |
| 224 | 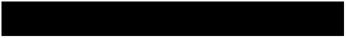 | −1.3 | −1.3 | 1.6 | 1 | −1 | −1.5 | −1.6 | −1.9 |
| 35 | 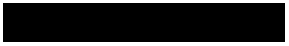 | −16 | −46 | −54 | −35 | −1.8 | −18 | −5.7 | −6.4 |
| 247 | AA433639_heat_shock_protein_cognate_70,_testis | 1.4 | −1.7 | −2.1 | −1.4 | 1.2 | −1 | 1.4 | 1.1 |
| 256 | AI046562_lactate_dehydrogenase_3,_C_chain,_sperm_specific | −4.9 | −1.8 | −8.8* | −1 | −1.1 | −3.8 | −4.2 | −4.4 |
| 259 | 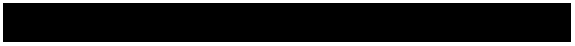 | −1.3 | −1.3 | 1.1 | −1.1 | −1.2 | −1.2 | −1.1 | −1.1 |
| 251 | 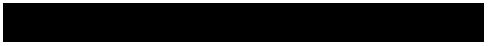 | −1.1 | −1.2 | −5.2 | −1.7 | −3.3 | −2.7 | −3 | −1.3 |
| 1 | 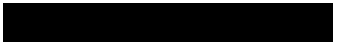 | 1.2 | −1.9 | −1.1 | −1.4 | 1.1 | [symbol] | 1.3 | −1.3 |
| 265 | 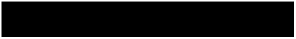 | −1.4 | −1.5 | 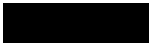 | 1.4 | −1.2 | −1.3 | −1.2 | −1.7 |
| 270 | AA062401_TATA_box_binding_protein_(TBP) | −5.2 | −3.7 | −1 | −15 | −3.4 | −3.8 | −4.8 | −5.5 |
| | associated_factor,_RNA_polymerase_II,_S,_150kD | | | | | | | | |
| 287 | AA600571_intracisternal_A_particles | −1.2 | −1.7 | −2.1 | −1.8 | −1.2 | −1.3 | −2.2 | −1.3 |
| 49 | AI550596_AI550596_stearoyl-Coenzyme_A_desaturase_2 | 1.4 | −1.1 | 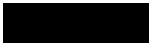 | 1.3 | 1.3 | −1.2 | −1.3 | −1.5 |
| 5 | 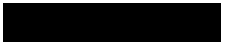 | −1 | −1.7 | 1.3 | −1.8 | 1 | −1.7 | 1 | −1.4 |
| 59 | AA591707_DNA_polymerase_epsilon,_subunit_2 | −1 | −1.2 | −1.9 | −1.2 | 1.1 | 1 | −1.2 | −1.3 |
| 71 | AA544433_dihydroorotate_dehydrogenase | 1.1 | −1.5 | 1.3 | −1.1 | 1.3 | −1.3 | 1.3 | −1.1 |
| 72 | AI037649_transition_protein_1 | −1.6 | −2.3 | −3.1 | −2.3 | −1.4 | −1.6 | −2.1 | −2.6 |
| 70 | AI553232_ribosomal_protein_S6 | 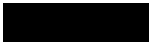 | 1.1 | 1.6 | −1.2 | 1.4 | 1.1 | 1.3 | −1.1 |
| 368 | 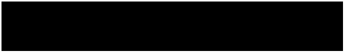 | 1.1 | 1.1 | −1.4 | 1.1 | 1.3 | 1.3 | 1.2 | 1.3 |
| 304 | 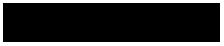 | 1.2 | −1 | 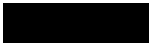 | 1.2 | 1.5 | 1.2 | 1.3 1.1 | |
| 398 | 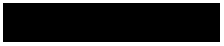 | 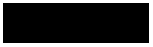 | 1 | 1.3 | −1.1 | 1.5 | 1.1 | 1.4 | 1.1 |
| 302 | 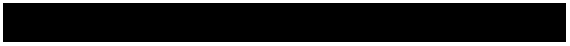 | 1.3 | 1.3 | 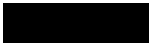 | 1.4 | 1.4 | 1.3 | 1.3 | 1.2 |
| 429 | 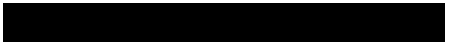 | 1.1+ | −1.2 | 1.5 | −1.2 | 1.2 | −1.2 | 1.1 | −1.1 |
| 402 | 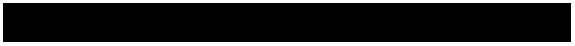 | 1.1 | −1.3 | 1.1 | −1.4 | 1.4 | −1 | 1.1 | −1.3 |
| 332 | 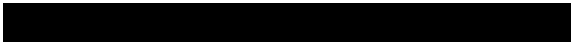 | 1.1+ | −1.1 | 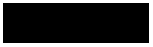 | 1.4 | 1.2 | 1 | 1.1+ | −1.6 |
| 383 | A1643201_Mus_musculus_TOB3_mRNA,_complete_cds | −1.2 | −2 | −1.5+ | −1.9 | −1.2 | −1.7 | 1.4 | −1.1 |
| 310 | AI549635_ESTs | 1 | −1.1 | −1.3 | 1.1 | 1.2 | −1.1 | −1.1 | −1.2 |
| 90 | 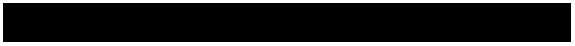 | 1.1 | −1 | 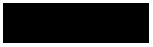 | 1.2 | 1.2 | 1.2 | 1 | −1 |
| 105 | 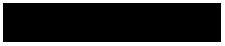 | 1.1 | −1.2 | 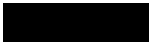 | 1.5 | 1.1* | −1.3 | 1.4 | 1.2 |
| 37 | 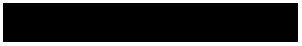 | −18 | −49 | −59 | −32 | −5.1 | −37 | −1.2* | −1.3 |
| 248 | AA388685_ESTs | 1.5+* | −1.4 | 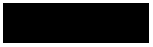 | 1.1 | 1.3+* | −1.3 | 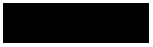 | 1.2 |
| 2 | AI549624_RIKEN_cDNA_061004E09_gene | 1.2 | −1.9 | −1.2 | −1.4 | 1.3 | −1.7 | 1.2 | −1.3 |
| 272 | 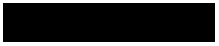 | 1.1 | −1.3 | 1 | −1 | 1.1 | −1.2 | 1.3 | −1.1 |
| 273 | 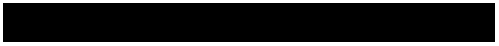 | 1.1 | −1.2 | 1.2* | 1 | 1.1 | −1.2 | 1.2 | −1.1 |
| 275 | 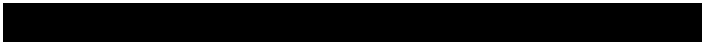 | 1.1 | −1.3 | −1 | −1.1 | 1 | −1.4 | 1.2 | 1 |
| 274 | 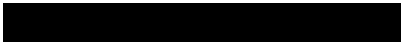 | 1 | −1.3 | 1 | −1.2 | 1 | −1.4 | 1.1 | −1 |
| 39 | AI876997_carboxyl_ester_lipase | −14 | −10 | −11 | −8.9 | −8.2 | −7.2 | −11 | −13 |
| 41 | 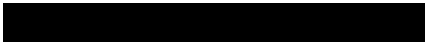 | −55 | −45 | −17 | −40 | −47 | −11 | −62 | −54 |
| 285 | AA982254_RIKEN_cDNA_2210010C04_gene | −19 | −13 | −9.7 | −15 | −23 | −11 | −30 | −21 |
| 288 | AA674409_pancreatic_lipase_related_protein_1 | −15 | −15 | −9.6 | −12 | −10 | −8.9 | −20 | −19 |
| 282 | AA675084_elastase_2 | −59 | −31 | −14 | −23 | −57 | −14 | −56 | [symbol] |
| 45 | AA155097_ESTs | 1.2* | −1.4 | 1.4 | 1 | 1.3 | −1.5 | 1.5 | 1.2 |
| 11 | AA153231_Public_domain_EST | 1.3 | −1.4 | 1.1* | −1.2 | 1.3 | [symbol] | 1.3 | −1.1 |
| 10 | AA561920_RalBPI_associated_Eps_domain_containing_protein | 1.4 | −1.3 | 1.6 | 1.3 | 1.4 | 1.4 | 1.5 | −1.1 |
| 13 | AA940207_calcium/calmodulin- | 1.2* | −1.4* | 1.6* | 1.3 | 1.3* | −1.5 | 1.6 | 1.2 |
| | dependent_protein_kinase_kinase_1,_alpha | | | | | | | | |
| 46 | AA469724_WW_domain_binding_protein_5 | 1.2 | −1.4 | −1.1 | −1.2 | 1.3 | [symbol] | 1.2 | −1.1 |
| 54 | 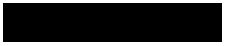 | 1.3 | −1.1 | 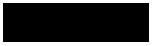 | 1.3 | 1.3 | −1.1 | 1.2 | −1 |
| 52 | 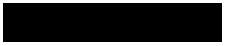 | 1.2 | −1.2 | 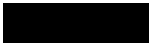 | −1.1* | 1.3 | −1.2 | 1.1 | −1.2 |
| 51 | 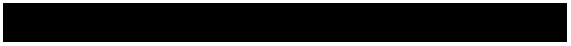 | 1.2 | −1.3 | 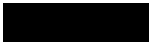 | 1.1 | 1.2 | −1.3 | 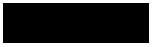 | 1.3 |
| 14 | 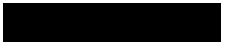 | 1.6 | −1.2 | 1 | −1.1 | [symbol] | −1.2 | 1.1 | −1.1 |
| 66 | 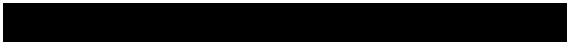 | 1.3 | −1.5 | 1.3* | 1 | 1.1 | −1.4 | 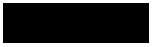 | 1.1 |
| 4 | 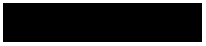 | 1.4* | −1.6 | 1.6 | −1.1 | 1.4* | −1.5 | 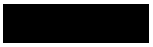 | 1 |
| 7 | 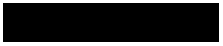 | 1.2 | −2 | 1.2 | −1.2 | 1.1* | −1.9 | 1.4 | −1.2 |
| 68 | 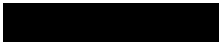 | 1 | [symbol] | 1.1 | −1.1 | 1.1 | −1.5 | 1.2 | −1.2 |
| 67 | 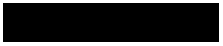 | 1.4 | −1.1 | 1.1 | −1.2 | 1.5 | −1.1 | −1.3 | 1 |
| 15 | 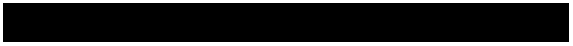 | 1.2* | −1.4 | 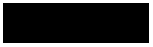 | −1.1 | 1.2 | −1.4 | 1.5 | 1 |
| 335 | 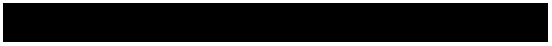 | 1.2 | −1.2 | 1.5 | 1 | 1.3 | −1.1 | 1.3 | 1.2 |
| 354 | 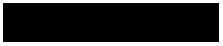 | 1.4* | −1.1 | 1.5 | 1 | 1.3 | −1.1 | 1.5 | 1.1 |
| 355 | 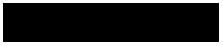 | 1.2 | 1.2 | 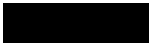 | 1.1 | 1.3 | −1 | 1.5 | −1.1* |
| 365 | 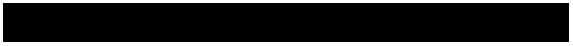 | 1.1 | −1.2 | 1.2 | −1.1 | 1.1 | −1.2 | 1.1 | −1 |
| 372 | 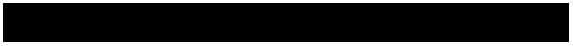 | 1.3 | −1.2 | 1.3 | −1.1 | 1.2 | −1.2 | 1.5 | 1.1 |
| 376 | 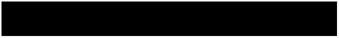 | 1.4 | −1.1 | 1.6 | 1 | 1.5 | −1.1 | 1.4 | 1 |
| 387 | AA671409_corel_UDP-galactose-N-acetylgalactosamine- | 1.4 | −1.3 | 1.6 | −1 | 1.4 | −1.1 | 1.3 | −1 |
| | alpha-R_beta_1,3-galactosyltransferase | | | | | | | | |
| 389 | 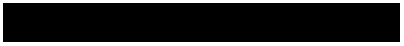 | 1.1 | −1.3 | 1.4 | −1.1 | 1.3 | −1.2 | 1.4 | 1.2 |
| 391 | 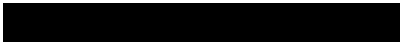 | 1.2 | −1.4 | 1.5 | 1.1 | 1.3 | −1.2 | 1.3 | −1 |
| 399 | 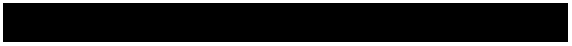 | 1.3* | −1.2 | 1.5 | 1.1 | 1.5 | −1.1 | 1.5 | 1 |
| 405 | 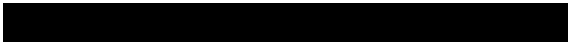 | 1.2 | −1.3 | 1.4 | −1 | 1.2 | 1.1 | 1.3 | 1.1 |
| 407 | 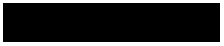 | 1.2* | −1.3 | 1.4 | 1.1 | 1.2 | −1.2 | 1.4 | 1.1 |
| 418 | 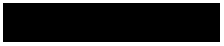 | 1.3 | −1.2 | 1.4 | 1.1 | 1.2 | −1 | 1.4 | 1.2 |
| 420 | AI463731_ESTs | 1.2 | −1.2 | 1.5 | −1 | 1.2 | −1.2 | [symbol] | −1.1 |
| 422 | AA273401_Public_domain_EST | 1.2 | −1.3 | 1.2 | −1 | 1.2* | −1.4 | 1 .4 | −1.1 |
| 374 | AI549656_RIKEN_cDNA_3632413F13_gene | 1.3 | −1.1 | [symbol] | −1.1 | 1.1 | −1 | 1.2 | −1 |
| 303 | AA688667_ESTs,_Weakly_similar_to_Inv_[M.musculus] | 1.3 | 1 | [symbol] | 1.2 | 1.4 | 1.2 | 1.1 | 1.1 |
| 388 | AA269742_RIKEN_cDNA_1110065A22_gene | 1.3 | 1 | 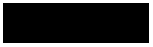 | 1.4 | 1.3 | 1 | 1.5 | 1.3 |
| 403 | AA220774_ESTs | 1.2 | −1.2 | −1 | 1 | 1.2 | −1.1 | 1.4 | 1 |
| 390 | AI587986_ESTs | 1.1 | −1.1 | 1.4+* | 1. | 1.3 | −1 | 1.1 | 1.2 |
| 377 | AI645426_diphanous_homolog_3_(Drosophila) | [symbol] | −1.2 | 1.5 | −1 | 1.5 | 1.1 | 1.2 | −1.1 |
| 380 | AI596550_leukemia/lymphoma_related_factor | 1.4 | −1.1 | 1.5 | 1.1 | 1.5 | −1 | 1.3 1.1 | |
| 424 | AA412912_ESTs | 1.3 | −1.2 | 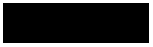 | −1.1 | 1.5 | −1.1 | 1.3 | 1 |
| 434 | AA244813_calbindin_2 | 1.2 | −1.2 | 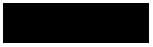 | 1.2 | 1.5 | 1.2 | 1.1 | −1.2 |
| 359 | AA116625_general_transcription_factor_IIH,_poly- | 1.2 | −1.2 | −1.1 | −1.1 | 1.1 | −1.2 | 1.5 | 1.3 |
| | peptide_2_(44_kDa_subunit) | | | | | | | | |
| 340 | AA823475_ESTs | 1.4 | −1 | 1.5 | 1.1 | 1.5 | 1.1 | 1.2 | 1.1 |
| 415 | AI604192_RIKEN_cDNA_4632433K11_gene | 1.1 | −1.1 | 1.2 | 1.1 | 1.2 | 1 | 1.4 | 1.3 |
| 412 | AA268472_RIKEN_cDNA_2410003C20_gene | 1.1 | −1.1 1.3* | 1.1 | 1.3 | 1.2 | 1.3 | 1.3 | |
| 382 | AI464600_ESTs | 1.4 | −1 | [symbol] | 1.2 | 1.4 | 1.2 | 1.6 | 1.2 |
| 314 | AA717080_ESTs | 1.2 | −1 | 1 | [symbol] | 1.5 | −1.2 | 1.2 | 1.1 |
| 370 | AA516997_kinase_suppressor_of_ras | 1.2 | −1 | 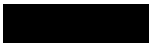 | 1 | 1.5 | 1 | 1.1 | −1.1 |
| 338 | AA591510_homeo_box_B1 | 1 | 1 | 1.5 | 1.2 | 1.2 | 1.1 | 1 | −1.1 |
| 409 | AA798385_ESTs | 1.5 | −1.1 | 1.3 | 1.1 | 1.3 | 1 | 1.2 | 1.1 |
| 432 | AI592746_ESTs | 1.3 | −1.2 | 1.1+* | 1.1 | 1.3 | −1 | 1.3 | 1.2 |
| 312 | AA646049_interleukin_13 | 1.3 | −1.1 | 1.5* | 1.1 | 1.5 | 1.1 | 1.3 | 1.1 |
| 331 | AA739464_killer_cell_lectin- | 1.2 | −1.2 | 1 | −1.1 | 1.2 | −1.2 | 1.3 | −1 |
| | like_receptor_subfamily_A,_member_9 | | | | | | | | |
| 334 | AI594144_DNA_segment,_Chr_3,_Brigham_&_Women's_Gene- | 1.4 | −1 | 1.2 | 1.1 | 1.4 | 1.1 | 1.2 | 1.1 |
| | tics_0878_expressed | | | | | | | | |
| 386 | AI627071_RIKEN_cDNA_2310058A11_gene | 1.2 | −1.3 | 1 | 1.1 | 1.2 | −1.1 | 1.3 | −1 |
| 419 | AI551677_ESTs | 1.1 | 1.1 | [symbol] | −1 | [symbol] | 1.1 | 1.4 | −1.1 |
| 319 | AA058211_ESTs | 1.3 | 1 | 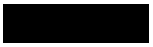 | 1.3 | 1.2 | 1.1 | 1.2 | −1.1 |
| 342 | AA615213_ESTs,_Moderately_similar_to_unnamed_pro- | 1.3 | −1.1 | [symbol] | 1.1 | 1.4 | −1 | 1.3 | 1.1 |
| | tein_product_[H.sapiens] | | | | | | | | |
| 313 | AA691755_RIKEN_cDNA_1110068E08_gene | 1.4 | 1.1 | 1.5 | 1.1 | 1.5 | 1.3 | 1.2 | 1.2 |
| 406 | AA290276_ESTs | 1.1 | −1.4 | 1.5 | 1.4 | 1.1 | [symbol] | 1.2 | 1 |
| 426 | 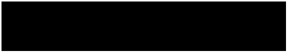 | [symbol] | −1.4 | 1.1 | −1 | 1.5 | 1 | 1.1 | 1 |
| 379 | AI594170_ESTs | [symbol] | −1.1 | [symbol] | 1.2 | 1.4 | 1.1 | 1.1 | 1.1 |
| 316 | AA067264_kenectin_1 | 1.3 | 1.2 | [symbol] | 1 | 1.4 | 1.1 | 1.1 | 1.1 |
| 392 | 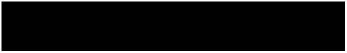 | 1.3 | −1.2 | 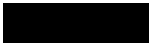 | 1.1 | 1* | −1.3 | 1.2 | −1 |
| 343 | AA608457_DNA_polymerase_epsilon | 1.4 | −1.2 | 1 | 1 | 1.4 | 1 | 1.1 | −1 |
| 306 | AA596430_galactose-phosphate_uridyl_transferase | 1.3 | −1.2 | 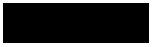 | 1.3 | 1.3 | −1 | 1.5 | 1.2 |
| 326 | AI789680_ESTs | 1.2 | −1 | [symbol] | 1.4 | [symbol] | 1.2 | [symbol] | 1.2 |
| 346 | AI607043_RIKEN_cDNA_2400007G07_gene | 1.2 | −1.1 | 1.5 | −1.1 | 1.4 | 1 | 1.5 | 1.1 |
| 361 | AW210270_ESTs,_Weakly_similar_to_T17254_hypo- thetical_protein_DKFZp586O1022.1_[H.sapiens] | 1.3 | −1.1 | 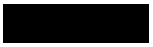 | 1.3 | 1.4 | −1 | 1.2 | −1.1* |
| 332 | AA266331_ESTs | 1.2 | −1.1 | 1.3 | 1.2 | 1.3 | 1.1 | 1.3 | 1.3 |
| 417 | AA543950_RIKEN_cDNA_2900073G15_gene | 1.3 | −1.1 | −1.2 | −1.1 | 1.4 | 1.1 | 1.3 | 1.1 |
| 362 | AI390609_ESTs | 1.4 | −1.2 | 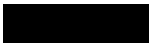 | 1.2 | 1.4 | 1.1 | 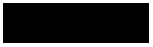 | 1* |
| 400 | AA517691_ESTs | 1.4 | −1.3 | 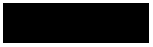 | 1 | 1.3 | −1.3 | 1.4 | −1.1 |
| 433 | AA638805_Public_domain_EST | 1.3 | −1.2 | 1.3* | −1 | 1.3 | −1.2 | 1.3 | 1 |
| 301 | AA709576_ESTs | 1.4+ | 1 | 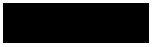 | 1.1 | 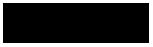 | 1.2 | 1.4 | 1 |
| 315 | AA709668_ESTs | 1.4 | −1 | 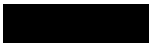 | 1.1 | 1.6 | 1.2 | 1.4 | 1.1 |
| 296 | AI614679_ESTs | 1.1 | 1.1 | 1.5 | −1 | 1.4 | 1.2 | 1.2 | −1 |
| 367 | AI450469_ESTs | 1.2 | −1.3 | 1.1* | −1 | 1.2 | 1.1 | 1.2 | −1 |
| 328 | AA616077_ESTs | 1.5 | −1.1 | 1.4 | 1.2 | 1.5 | 1.2 | 1.2 | 1.1 |
| 350 | AA268913_flavin_containing_monooxygenase_5 | [symbol] | 1.1 | [symbol] | 1.2 | [symbol] | 1 | 1.5 | [symbol] |
| 351 | AA210560_Mus_musculus,_Similar_to_RIKEN_cDNA_1 | 1.1+* | −1.5 | [symbol] | 1 | 1.1 | [symbol] | 1.4 | 1 |
| | 500041N16_gene,_clone_MGC:12066,_mRNA,_complete_cds | | | | | | | | |
| 371 | AI607675_Mus_musculus,_clone_MGC:7865,_mRNA_com- plete_cds | [symbol] | [symbol] | 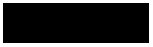 | 1.1 | [symbol] | −1.1 | [symbol] | 1.2 |
| 394 | AA265095_ESTs | [symbol] | −1.2 | 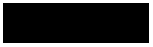 1.1 | 1.2 | −1.1 | 1.5 | 1.2 | |
| 404 | AA727914_ESTs | 1.4 | −1.3 | 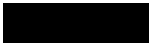 | −1* | 1.5 | −1.2 | 1.5 | 1.2 |
| 423 | AA204045_Public_domain_EST | 1.1* | [symbol] | 1.2 | 1.3 | 1.2 | −1.4 | 1.4 | 1 |
| 435 | AA623587_ESTs | 1.3 | −1.2 | 1.1 | −1.1 | 1.3 | −1.2 | 1.3 | 1.1 |
| 444 | AA259281_LIM_homeobox_protein_2 | 1.1 | −1.1 | [symbol] | 1 | 1.1 −1.3 | [symbol] | 1.2 | |
| 324 | AA822679_homatopoietic_cell_signal_transducer | 1.1 | −1.1 | 1.2 | −1 | 1.1 | 1 | 1.2 | 1 |
| 410 | AA473386_ESTs | [symbol] | −1.1 | 1.2 | 1.1 | 1.5 | 1.1 | 1.2 | −1.1 |
| 318 | AA259871_hypothetical_protein,_MNCb-4414 | [symbol] | −1.1 | [symbol] | 1.2 | 1.5 | 1.1 | 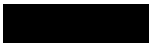 | 1.4 |
| 425 | AA474374_RIKEN_cDNA_5730408C10_gene | 1.2 | −1.1 | 1.5 | 1 | 1.1 | −1.2 | 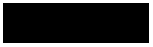 | 1.2 |
| 414 | AA230591_myosin,_heavy_poly- | 1.1* | −1.6 | 1.4 | 1 | 1.1* | −1.5 | 1.3 | −1 |
| | peptide_3,_skeletal_muscle_embryonic | | | | | | | | |
| 427 | AA607267_Public_domain_EST | 1.3 | −1.4 | [symbol] | −1.2 | 1.4 | [symbol] | 1.4 | −1.1 |
| 369 | AA469648_growth_factor,_erv1_(S._cerevisiae)- | −1 | −1.5 | 1.3 | −1.2 | 1.2 | [symbol] | [symbol] | 1 |
| | like_(augumenter_of_liver_regeneration) | | | | | | | | |
| 323 | AA060731_nuclear_factor,_interleukin_3,_regulated | 1 | −1.4 | 1 | 1 | 1.2 | −1.3 | 1.2 | −1.1 |
| 347 | AA638198_gap_junction_membrane_channel_protein_beta_3 | 1 | [symbol] | 1.2 | −1.2 | −1.1 | −1.4 | 1.1 | [symbol] |
| 393 | AI530661_RIKEN_cDNA_2810426N06_gene | 1.1 | −1.2 | 1.2+ | 1.1 | 1.2 | [symbol] | 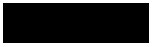 | [symbol] |
| 358 | AI466825_RIKEn_cDNA_A330103N21_gene | 1.5 | 1 | [symbol] | 1.6 | 1.2 | 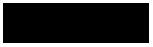 | 1.2 | 1.2 |
| 421 | AA162023_ESTs | 1.1* | [symbol] | [symbol] | −1.1 | 1.2 | −1.6 | 1.2 | −1.1 |
| 329 | AA611551_Mest-linked_imprinted_transcript_1 | 1.4 | −1.1 | 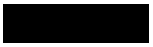 | 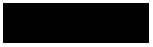 | 1.4 | 1 | 1.1 | 1.2 |
| 436 | AA277192_RIKEN_cDNA_2900075A18_gene | −1 | −1.4 | 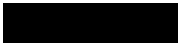 | 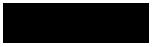 | 1.2 | 1.1 | 1 | −1.2 |
| 291 | AI117905_RIKEN_cDNA_6530406M24_gene | −1.2 | 1.3 | 1.5 | 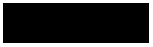 | −1.1 | 1.1 | 1.2 | 1.3 |
| 281 | AI550886_RIKEN_cDNA_1810010M01_gene | −1.9 | −3.1 | −3.1 | −2.8 | −1.8 | −2.1 | −2.6 | −3.1 |

| | | 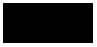 | | 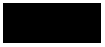 | | | | |
|---|---|---|---|---|---|---|---|---|
| | | (−) | (+) | (−) | (+) | | | |
| IDI | Description | PL-11B_BDE | PL-23B_BDE | PL-12B_BDE | PL-24B_BDE | Max Signal | Arrange Signal | Missed Values |
| 244 | AA726092_RIKEN_cDNA_4933419D20_gene | 1.5 | 1.4 | −1.3 | −1.2 | 1191 | [symbol] | 0 |
| 289 | AA222208_surfactant_associated_protein_C | −15 | −19 | −14 | −24 | 57182 | [symbol] | 0 |
| 366 | AA710379_neuroplin | 1.1* | −1.2 | 1.3 | −1.1 | [symbol] | [symbol] | 2 |
| 344 | AI158775_protein_tyrosine_phosphatase,_receptor- | 1.2* | 1.1 | 1.4 | 1 | 1416 | 437 | 3 |
| | type,_M | | | | | | | |
| 204 | AA929863_ESTs,_similar_to_T46271_hypo- | 1.3 | 1.6** | 1.2 | 1.4 | [symbol] | [symbol] | 0 |
| | thetical_protein_DKFZp564P1263.1_[H.sapiens] | | | | | | | |
| 157 | AI892437_transglutaminase_2,_C_polypeptide | 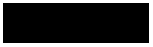 | 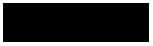 | 1.3 | 1.4 | [symbol] | [symbol] | 0 |
| 203 | W18463_thioether_S-methyltransferase | −7.8 | −7.7 | −5.8 | −1.8 | [symbol] | 1920.75 | 0 |
| 200 | AI117310_flavin_containing_monooxygenase_1 | −2.1 | −1.5 | −1.4 | −1.1 | 9215 | [symbol] | 0 |
| 135 | AA670943_phosphatidic_acid_phosphotase_2a | 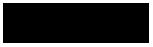 | 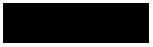 | −1.5 | −1.4 | [symbol] | [symbol] | 0 |
| 172 | AI552087_IQ_motif_containing_GTPase_activating_protein_1 | 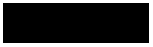 | 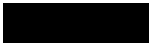 | 1.3 | 1.2 | [symbol] | [symbol] | 0 |
| 36 | AA466852_cysteine_rich_protein_61 | 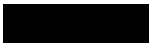 | 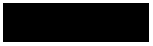 | −1.1 | −1.2 | [symbol] | [symbol] | 1 |
| 16 | 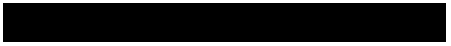 | −8.4 | −14 | −7.6 | −17 | [symbol] | [symbol] | 0 |
| 79 | 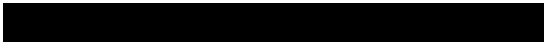 | −21 | −23 | −21 | −23 | [symbol] | [symbol] | 0 |
| 74 | 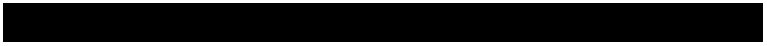 | −22 | −19 | −26 | −15 | 1773 | [symbol] | 0 |
| 75 | 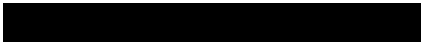 | −36 | −29 | −12 | −29 | [symbol] | [symbol] | 0 |
| 110 | 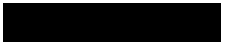 | 1.5 | 1.3 | 1.6 | 1.3 | [symbol] | [symbol] | 4 |
| 89 | 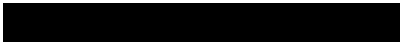 | −1.1 | −1.2 | −1.1 | −1.1 | [symbol] | [symbol] | 7 |
| 102 | 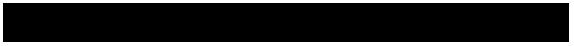 | 1.4 | −1.1 | 1.4 | −1.1 | 1351 | [symbol] | 1 |
| 86 | 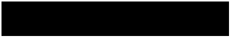 | −1.1 | −1 | −1.2 | 1 | 1033 | [symbol] | 0 |
| 103 | 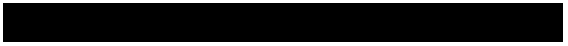 | 1.1 | 1.2 | 1.2 | 1.1+ | [symbol] | [symbol] | 11 |
| 93 | 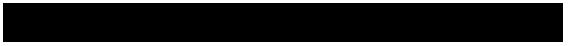 | 1.1 | 1 | 1.2 | 1.1 | [symbol] | [symbol] | 1 |
| 18 | AA270885_parvalbumin | −3.8 | −9 | −1 | 1.2 | [symbol] | [symbol] | 0 |
| 110 | AA763276_small_protein,_X-linked | −2.1 | −1 | −1.4 | 1.1 | [symbol] | [symbol] | 0 |
| 121 | AA671135_myosin_heavy_chain,_cardiac_muscle,_adult | −9.1 | −7.5 | −3.5 | −2.8 | 2711 | [symbol] | 0 |
| 126 | AA795264_ubiquitin-like_1 | −4.4 | −4 | −4.2 | −3.6 | [symbol] | [symbol] | 0 |
| 127 | AI893944_forkhead_box_G1 | −1.3 | 1.2 | −1.5 | −1.3 | [symbol] | [symbol] | 0 |
| 108 | 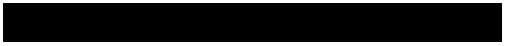 | 1.1 | 1.1 | −3.1 | −1.9 | [symbol] | [symbol] | 0 |
| 146 | 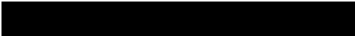 | '16 | −9.3 | −6.9 | −8.3 | [symbol] | [symbol] | 0 |
| 153 | AA770902_actin,_alpha_1,_skeletal_muscle | −2.2 | −2.1 | 1.3 | 1.6 | [symbol] | [symbol] | 0 |
| 182 | AA821997_myoglobin | −24 | −21 | −3.8 | −2.2 | [symbol] | [symbol] | 0 |
| 192 | 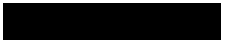 | 1.2 | 1.4 | 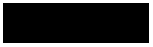 | 1.4 | [symbol] | [symbol] | 8 |
| 216 | AA712088_3-hydroxy-3-methylglutaryl- | −2.5 | −2.1 | −3.6 | −3.6 | [symbol] | [symbol] | 1 |
| | Coenzyme_A_reductase | | | | | | | |
| 221 | 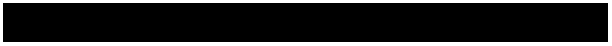 | 1.1 | 1.1 | 1.1* | −1 | [symbol] | 371.75 | 3 |
| 234 | 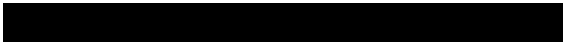 | −1.6 | −2 | 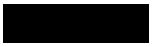 | 1.2 | [symbol] | [symbol] | 3 |
| 231 | 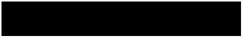 | −1.3 | −1.7* | 1.3 | −1.1 | [symbol] | [symbol] | 10 |
| 224 | 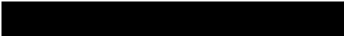 | 1 | 1.1 | −1.1 | −1.2 | [symbol] | [symbol] | 0 |
| 35 | 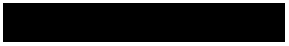 | −35 | −33 | −72 | −52 | 8502 | [symbol] | 0 |
| 247 | AA433639_heat_shock_protein_cognate_70,_testis | 1.2 | −1.1 | −1 | [symbol] | [symbol] | [symbol] | 0 |
| 256 | AI046562_lactate_dehydrogenase_3,_C_chain,_sperm_specific | −2.7 | −4.8 | −2.1 | −5 | [symbol] | [symbol] | 0 |
| 259 | 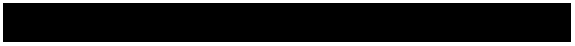 | −1.1 | 1 | 1.1 | 5.2*** | [symbol] | [symbol] | 0 |
| 251 | 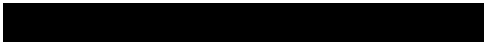 | −3.7 | −4.4 | −2.9 | −5.7 | [symbol] | [symbol] | 0 |
| 1 | 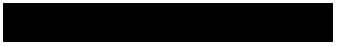 | 1.4 | −1.6 | 1.4 | −1.6 | 1797 | [symbol] | 1 |
| 265 | 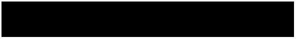 | −1 | −1.1 | −1.2 | −1.2 | [symbol] | [symbol] | 0 |
| 270 | AA062401_TATA_box_binding_protein_(TBP) | −7.2 | −4.5 | 1 | −1.2 | 15483 | [symbol] | 0 |
| | associated_factor,_RNA_polymerase_II,_S,_150kD | | | | | | | |
| 287 | AA600571_intracisternal_A_particles | 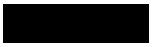 | 1.2 | −1.7 | −2.3 | [symbol] | [symbol] | 0 |
| 49 | AI550596_AI550596_stearoyl-Coenzyme_A_desaturase_2 | 1.1 | −1.3 | 1.1 | −1.1 | [symbol] | [symbol] | 0 |
| 5 | 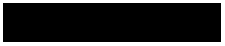 | 1.2 | −1.7 | 1.3 | −1.6 | [symbol] | [symbol] | 2 |
| 59 | AA591707_DNA_polymerase_epsilon,_subunit_2 | 1 | 1.1 | 1 | −1.1 | 2511 | [symbol] | 0 |
| 71 | AA544433_dihydroorotate_dehydrogenase | 1.4 | −1.5 | 1.5 | −1.1 | 1328 | [symbol] | 1 |
| 72 | AI037649_transition_protein_1 | −1.5 | −2.5 | −1.4 | −2.4 | [symbol] | [symbol] | 0 |
| 70 | AI553232_ribosomal_protein_S6 | 1.5 | −1.4 | 1.4 | −1.2 | [symbol] | [symbol] | 1 |
| 368 | 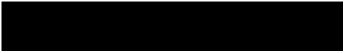 | 1.2 | −1.1 | 1.3* | −1.1 | 1642 | [symbol] | 2 |
| 304 | 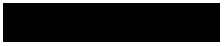 | 1.5 | 1.4 | 1.3 | 1.2 | 1147 | 314.375 | 2 |
| 398 | 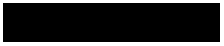 | 1.4 | −1.1 | 1.4 | −1.1 | [symbol] | [symbol] | 1 |
| 302 | 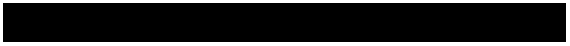 | 1.1 | 1 | 1.2* | 1.1 | [symbol] | 407.875 | 2 |
| 429 | 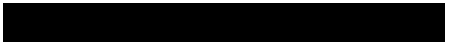 | 1.3 | −1.1 | 1.4 | −1.2 | [symbol] | 241 | 5 |
| 402 | 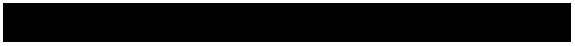 | 1.1 | −1.3 | 1.2 | −1.2 | [symbol] | [symbol] | 0 |
| 332 | 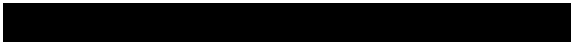 | 1.3 | −1.2 | 1.1 | −1.1 | 619 | [symbol] | 7 |
| 383 | A1643201_Mus_musculus_TOB3_mRNA,_complete_cds | 1.1 | −1.4 | 1.2 | −1.2 | [symbol] | [symbol] | 2 |
| 310 | AI549635_ESTs | 1.2 | −1.2 | 1.3 | 1.2 | [symbol] | [symbol] | 2 |
| 90 | 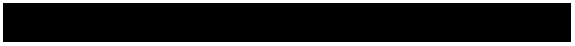 | 1.1 | 1.1 | 1.1 | 1.2 | [symbol] | 714.75 | 1 |
| 105 | 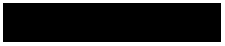 | 1.2* | −1.1 | 1.2** | −1 | [symbol] | [symbol] | 0 |
| 37 | 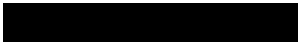 | −14 | −14 | −39 | −51 | [symbol] | [symbol] | 0 |
| 248 | AA388685_ESTs | 1.5* | −1.2 | 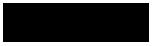 | −1 | [symbol] | [symbol] | 3 |
| 2 | AI549624_RIKEN_cDNA_061004E09_gene | 1.3 | −1.8 | 1.5 | −1.5 | [symbol] | [symbol] | 1 |
| 272 | 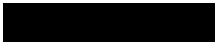 | 1.2 | −1.1 | 1.2* | −1.1 | [symbol] | [symbol] | 0 |
| 273 | 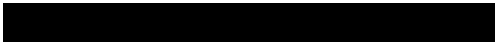 | 1.1 | −1.2 | 1.2 | −1.1 | [symbol] | [symbol] | 0 |
| 275 | 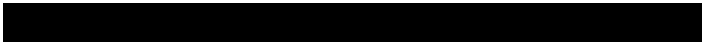 | 1.1 | −1.2 | 1.1 | −1.2 | [symbol] | [symbol] | 0 |
| 274 | 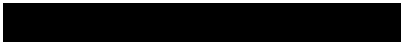 | 1.1 | −1.3 | 1.2 | −1.2 | [symbol] | [symbol] | 0 |
| 39 | AI876997_carboxyl_ester_lipase | −6.7 | −14 | −5.6 | −12 | [symbol] | [symbol] | 0 |
| 41 | 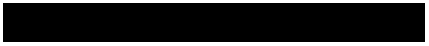 | −54 | −51 | −60 | −49 | [symbol] | [symbol] | 0 |
| 285 | AA982254_RIKEN_cDNA_2210010C04_gene | −20 | −19 | −25 | −22 | [symbol] | [symbol] | 0 |
| 288 | AA674409_pancreatic_lipase_related_protein_1 | −10 | −16 | −11 | −15 | [symbol] | [symbol] | 0 |
| 282 | AA675084_elastase_2 | −15 | −39 | −54 | −39 | [symbol] | [symbol] | 0 |
| 45 | AA155097_ESTs | 1.4 | −1.3 | 1.4 | −1.2 | [symbol] | [symbol] | 0 |
| 11 | AA153231_Public_domain_EST | 1.3 | −1.5 | 1.5 | [symbol] | 2724 | [symbol] | 0 |
| 10 | AA561920_RalBPI_associated_Eps_domain_containing_protein | 1.6 | −1.3 | 1.4 | −1.2 | [symbol] | [symbol] | 0 |
| 13 | AA940207_calcium/calmodulin- | 1.4* | 1.2* | [symbol] | −1.2* | [symbol] | [symbol] | 0 |
| | dependent_protein_kinase_kinase_1,_alpha | | | | | | | |
| 46 | AA469724_WW_domain_binding_protein_5 | 1.2 | −1.3 | 1.3 | −1.3 | [symbol] | [symbol] | 0 |
| 54 | 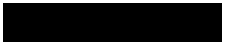 | 1.2 | −1.2 | 1.3 | −1.3 | [symbol] | [symbol] | 0 |
| 52 | 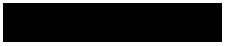 | 1.4 | −1.1 | 1.4 | −1.2 | [symbol] | [symbol] | 1 |
| 51 | 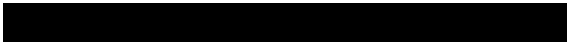 | 1.6 | −1 | 1.6* | 1 | [symbol] | [symbol] | 0 |
| 14 | 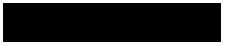 | 1.3 | −1.4 | 1.3 | −1.4 | 3117 | [symbol] | 0 |
| 66 | 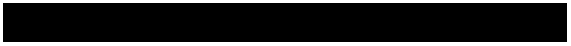 | 1.5 | −1.2 | 1.6* | −1.1 | [symbol] | [symbol] | 0 |
| 4 | 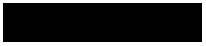 | 1.6 | −1.5* | [symbol] | −1.2 | 1512 | [symbol] | 1 |
| 7 | 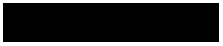 | 1.4* | −1.6 | 1.3 | −1.3 | [symbol] | 452.875 | 1 |
| 68 | 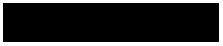 | 1.2 | −1.5 | 1.4 | −1.4 | [symbol] | [symbol] | 1 |
| 67 | 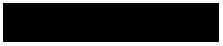 | 1.4 | −1.3 | 1.4 | −1.3 | 1571 | [symbol] | 1 |
| 15 | 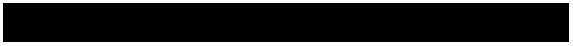 | −1.5 | −1.3 | 1.6* | −1.2 | [symbol] | [symbol] | 0 |
| 335 | 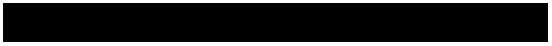 | 1.2 | 1 | 1.2 | −1 | [symbol] | 1024.75 | 1 |
| 354 | 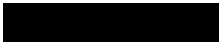 | 1.3 | −1.1 | 1.3 | 1 | 2474 | [symbol] | 1 |
| 355 | 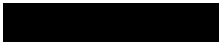 | [symbol] | −1.2 | 1.1 | 1.1 | [symbol] | [symbol] | 1 |
| 365 | 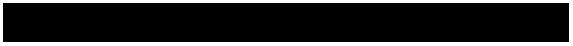 | 1.2 | −1.2 | 1.2 | −1.1 | [symbol] | [symbol] | 1 |
| 372 | 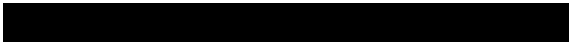 | 1.3 | −1.2 | 1.3 | −1.1 | [symbol] | [symbol] | 0 |
| 376 | 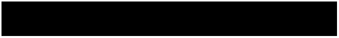 | 1.4 | −1.1 | 1.4 | −1 | [symbol] | [symbol] | 1 |
| 387 | AA671409_corel_UDP-galactose-N-acetylgalactosamine- | 1.4 | −1.1 | 1.3 | −1.1 | 2618 | 845.792 | 1 |
| | alpha-R_beta_1,3-galactosyltransferase | | | | | | | |
| 389 | 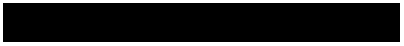 | 1.1 | −1.2 | 1.3 | −1.1 | 1526 | [symbol] | 1 |
| 391 | 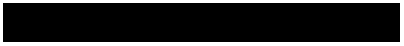 | 1.3 | −1.2 | 1.2 | −1.2 | [symbol] | [symbol] | 1 |
| 399 | 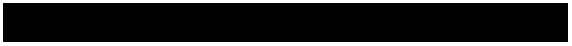 | 1.3 | −1.1 | 1.4 | −1.1 | 2240 | 998.125 | 0 |
| 405 | 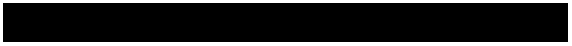 | 1.2 | −1.2 | 1.3 | −1.2 | [symbol] | 1012.33 | 0 |
| 407 | 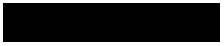 | 1.4 | −1.2 | 1.4 | −1.1 | [symbol] | 871.417 | 0 |
| 418 | 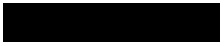 | 1.3 | −1.1 | 1.3 | −1.2 | 2032 | 718.75 | 1 |
| 420 | AI463731_ESTs | 1.2 | −1.3 | 1.3 | −1.1 | [symbol] | [symbol] | 1 |
| 422 | AA273401_Public_domain_EST | 1.4* | −1.2 | 1.4* | −1.2 | [symbol] | [symbol] | 0 |
| 374 | AI549656_RIKEN_cDNA_3632413F13_gene | 1.2 | −1 | 1.3 | −1.1 | [symbol] | [symbol] | 0 |
| 303 | AA688667_ESTs,_Weakly_similar_to_Inv_[M.musculus] | 1.2* | −1 | 1.1* | 1.1 | [symbol] | [symbol] | 2 |
| 388 | AA269742_RIKEN_cDNA_1110065A22_gene | 1.1 | 1 | 1.4 | −1 | [symbol] | [symbol] | 1 |
| 403 | AA220774_ESTs | 1.2 | −1.3 | 1.3 | −1.2 | [symbol] | [symbol] | 0 |
| 390 | AI587986_ESTs | 1.2 | −1.1 | 1.3 | −1.2 | [symbol] | [symbol] | 2 |
| 377 | AI645426_diphanous_homolog_3_(Drosophila) | 1.3 | −1 | 1.3 | −1.1 | [symbol] | 954.75 | 1 |
| 380 | AI596550_leukemia/lymphoma_related_factor | 1.2 | −1.2 | 1.2 | −1.2 | [symbol] | [symbol] | 1 |
| 424 | AA412912_ESTs | 1.3 | −1.1 | 1.4 | −1.2 | [symbol] | [symbol] | 1 |
| 434 | AA244813_calbindin_2 | 1.3 | −1.2 | 1.3 | −1.3 [symbol] | 585 | 1 | |
| 359 | AA116625_general_transcription_factor_IIH,_poly- | 1.2 | −1.1 | 1.2 | −1.1 | [symbol] | [symbol] | 0 |
| | peptide_2_(44_kDa_subunit) | | | | | | | |
| 340 | AA823475_ESTs | 1.3* | 1 | 1.4 | 1.1 | [symbol] | [symbol] | 2 |
| 415 | AI604192_RIKEN_cDNA_4632433K11_gene | 1.3 | −1.1 | 1.3 | −1.2 | [symbol] | [symbol] | 1 |
| 412 | AA268472_RIKEN_cDNA_2410003C20_gene | 1.2 | −1.1 | 1.3 | −1.2 | [symbol] | [symbol] | 1 |
| 382 | AI464600_ESTs | 1.2 | −1.1 | 1.4 | −1 | [symbol] | [symbol] | 1 |
| 314 | AA717080_ESTs | 1.2 | 1 | 1.3 | 1.1 | [symbol] | [symbol] | 1 |
| 370 | AA516997_kinase_suppressor_of_ras | 1.3 | 1 | 1.4 | −1 | [symbol] | [symbol] | 1 |
| 338 | AA591510_homeo_box_B1 | 1.2 | 1.1 | 1.1 | −1.1 | 2574 | [symbol] | 1 |
| 409 | AA798385_ESTs | 1.3 | −1.1 | 1.3 | −1.2 | 1513 | [symbol] | 1 |
| 432 | AI592746_ESTs | 1.2 | −1.2 | 1.3 | −1.3 | [symbol] | [symbol] | 2 |
| 312 | AA646049_interleukin_13 | 1.3 | −1.1 | 1.3 | 1.1 | [symbol] | [symbol] | 1 |
| 331 | AA739464_killer_cell_lectin- | 1.2 | −1.1 | 1.1 | −1.1 | 3824 | 1133.67 | 0 |
| | like_receptor_subfamily_A,_member_9 | | | | | | | |
| 334 | AI594144_DNA_segment,_Chr_3,_Brigham_&_Women's_Gene- | 1.3 | 1 | 1.4 | 1.1 | [symbol] | [symbol] | 0 |
| | tics_0878_expressed | | | | | | | |
| 386 | AI627071_RIKEN_cDNA_2310058A11_gene | 1.2 | −1.2 | 1.2 | −1.2 | [symbol] | [symbol] | 1 |
| 419 | AI551677_ESTs | 1.2 | −1 | [symbol] | −1.1 | [symbol] | [symbol] | 1 |
| 319 | AA058211_ESTs | 1.1 | −1.1 | 1.2 | −1 | 3419 | [symbol] | 0 |
| 342 | AA615213_ESTs,_Moderately_similar_to_unnamed_pro- | 1.3 | −1.1 | 1.3 | −1 | [symbol] | [symbol] | 2 |
| | tein_product_[H.sapiens] | | | | | | | |
| 313 | AA691755_RIKEN_cDNA_1110068E08_gene | 1.3* | 1 | 1.3** | 1.1 | 2427 | [symbol] | 3 |
| 406 | AA290276_ESTs | 1.2 | −1.3 | 1.2 | −1.3 | [symbol] | [symbol] | 1 |
| 426 | 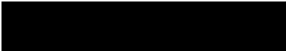 | 1.2* | −1.2 | 1.4 | −1.2 | [symbol] | 413.417 | 2 |
| 379 | AI594170_ESTs | 1.2 | −1.1 | 1.3 | −1.1 | 2855 | [symbol] | 0 |
| 316 | AA067264_kenectin_1 | 1.2* | 1.2 | 1.3* | 1 | [symbol] | [symbol] | 1 |
| 392 | 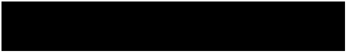 | 1.4* | −1.2 | 1.3 | −1.1 | 1484 | [symbol] | 1 |
| 343 | AA608457_DNA_polymerase_epsilon | 1.3 | −1.2 | 1.4 | 1.1 | 1473 | [symbol] | 1 |
| 306 | AA596430_galactose-phosphate_uridyl_transferase | 1.3 | −1.1 | 1.3 | 1.2 | [symbol] | [symbol] | 1 |
| 326 | AI789680_ESTs | [symbol] | [symbol] | [symbol] | 1.1 | [symbol] | [symbol] | 2 |
| 346 | AI607043_RIKEN_cDNA_2400007G07_gene | 1.3 | −1 | 1.3 | −1 | [symbol] | [symbol] | 1 |
| 361 | AW210270_ESTs,_Weakly_similar_to_T17254_hypo- | 1.2 | −1.2 | 1.2 | −1.1 | [symbol] | [symbol] | 0 |
| | thetical_protein_DKFZp586O1022.1_[H.sapiens] | | | | | | | |
| 332 | AA266331_ESTs | 1.2* | −1 | 1.2 | −1 | 1650 | 477.375 | 2 |
| 417 | AA543950_RIKEN_cDNA_2900073G15_gene | 1.3 | −1.1 | 1.3 | −1.2 | [symbol] | [symbol] | 0 |
| 362 | AI390609_ESTs | 1.4 | −1.1 | 1.3 | −1 | [symbol] | [symbol] | 1 |
| 400 | AA517691_ESTs | 1.4 | −1.3 | 1.4 | −1.1 | [symbol] | [symbol] | 0 |
| 433 | AA638805_Public_domain_EST | 1.3 | −1.2 | 1.4 | −1.2 [symbol] | [symbol] | 1 | |
| 301 | AA709576_ESTs | 1.2* | 1.1 | 1.3* | 1.2 | 1521 | 152.458 | 3 |
| 315 | AA709668_ESTs | 1.2* | 1 | 1.3* | 1 | [symbol] | [symbol] | 2 |
| 296 | AI614679_ESTs | 1.2 | 1.1 | 1.4 | 1.4 | 3573 | 1194.83 | 0 |
| 367 | AI450469_ESTs | 1.1 | −1.3 | 1.2 | −1.1 | [symbol] | 1103.5 | 0 |
| 328 | AA616077_ESTs | 1.3* | −1.1 | 1.3* | 1 | [symbol] | [symbol] | 1 |
| 350 | AA268913_flavin_containing_monooxygenase_5 | 1.2 | 1 | 1.4 | 1 | [symbol] | 413.5 | 3 |
| 351 | AA210560_Mus_musculus,_Similar_to_RIKEN_cDNA_1 | 1.3 | −1.1 | 1.3 | −1 | [symbol] | [symbol] | 2 |
| | 500041N16_gene,_clone_MGC:12066,_mRNA,_complete_cds | | | | | | | |
| 371 | AI607675_Mus_musculus,_clone_MGC:7865,_mRNA_com- | 1.4 | −1.1 | 1.4 | 1 | [symbol] | [symbol] | 0 |
| | plete_cds | | | | | | | |
| 394 | AA265095_ESTs | 1.3 | −1.2 | 1.1 | −1.1 | 1523 | [symbol] | 1 |
| 404 | AA727914_ESTs | 1.4 | −1.1 | 1.5 | −1.1 | 1615 | [symbol] | 1 |
| 423 | AA204045_Public_domain_EST | 1.2 | −1.3 | 1.3 | −1.3 | [symbol] | [symbol] | 0 |
| 435 | AA623587_ESTs | 1.4 | −1.1 | 1.4 | −1.2 | [symbol] | [symbol] | 1 |
| 444 | AA259281_LIM_homeobox_protein_2 | 1.4* | −1.1 | 1.4* | −1.1* | [symbol] | [symbol] | 0 |
| 324 | AA822679_homatopoietic_cell_signal_transducer | 1.2 | −1 | 1.3 | 1 | [symbol] | [symbol] | 1 |
| 410 | AA473386_ESTs | 1.3 | −1.2 | 1.3 | −1.2 | [symbol] | 777.167 | 1 |
| 318 | AA259871_hypothetical_protein,_MNCb-4414 | 1.3 | 1.1 | 1.4 | 1.1 | [symbol] | [symbol] | 1 |
| 425 | AA474374_RIKEN_cDNA_5730408C10_gene | 1.5 | 1 | 1.6 | 1 | [symbol] | 743.667 | 0 |
| 414 | AA230591_myosin,_heavy_poly- | 1.2 | −1.2 | 1.4 | −1.1 | 1479 | [symbol] | 1 |
| | peptide_3,_skeletal_muscle_embryonic | | | | | | | |
| 427 | AA607267_Public_domain_EST | 1.3 | −1.3 | 1.4 | −1.2 | 2112 | [symbol] | 1 |
| 369 | AA469648_growth_factor,_erv1_(S._cerevisiae)- | 1.2 | −1.2 | 1.3 | −1 | 2435 | [symbol] | 1 |
| | like_(augumenter_of_liver_regeneration) | | | | | | | |
| 323 | AA060731_nuclear_factor,_interleukin_3,_regulated | 1.1 | 1.1 | 1.2 | −1 | [symbol] | [symbol] | 1 |
| 347 | AA638198_gap_junction_membrane_channel_protein_beta_3 | 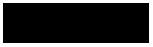 | 1.3 | 1.1 | −1.2 | [symbol] | [symbol] | 1 |
| 393 | AI530661_RIKEN_cDNA_2810426N06_gene | 1.5 | −1.1 | 1.6* | 1.1 | 559 | [symbol] | 1 |
| 358 | AI466825_RIKEn_cDNA_A330103N21_gene | 1.2 | −1.2 | 1.2 | −1.1 | [symbol] | [symbol] | 0 |
| 421 | AA162023_ESTs | 1.2 | −1.4 | 1.2 | −1.3 | [symbol] | [symbol] | 1 |
| 329 | AA611551_Mest-linked_imprinted_transcript_1 | 1.2* | −1.2 | 1.3 | 1 | 1612 | [symbol] | 2 |
| 436 | AA277192_RIKEN_cDNA_2900075A18_gene | 1.1 | −1.4 | 1.2 | −1.5 | 2277 | [symbol] | 1 |
| 291 | AI117905_RIKEN_cDNA_6530406M24_gene | −1.1 | 1.3 | −1.4 | −1.1 | [symbol] | [symbol] | 2 |
| 281 | AI550886_RIKEN_cDNA_1810010M01_gene | −1.6 | −3.2 | −1.6 | −2.9 | [symbol] | [symbol] | 0 |

| | | | | | 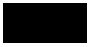 | | 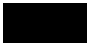 | |
|---|---|---|---|---|---|---|---|---|
| | | | | | (−) | (+) | (−) | (+) |
| IDI | Description | No. Tiss | 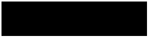 | 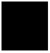 | PL-4B_BDE | PL-16B_BDE | PL-2B_BDE | PL-14B_BDE |
| 225 | AA116636_RIKEN_cDNA_1110013A05_gene | 1 | liver | panc, col, brain, pl/ut | −1.1 | [symbol] | 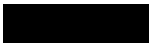 | 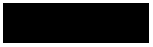 |
| 284 | AA237793_rat_regenerating_islet- derived,_mouse_homolog_1 | 1 | spleen | panc, int | −3.7 | −9.3 | 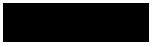 | 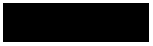 |
| 28 | AA242201_serine_protease_inhibitor,_Kazal_type_3 | 2 | intestine; mammary gland | panc, int, col, blad, kidn | −4.7 | −6.9 | 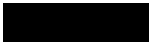 | 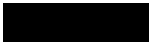 |
| 201 | AA867621_S-phase_kinase-associated_protein_2_(p45) | 1 | liver | panc, liv | −1.3 | 1.4 | 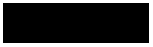 | 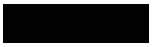 |
| 232 | AI893740_glycine_N-methyltransferase | 1 | liver | panc, liv | −1.6 | −1.7 | 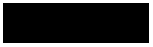 | 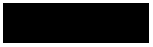 |
| 353 | AA684073_ESTs | 1 | stomach | panc, liv | −1 | −1 | 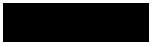 | 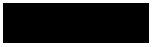 |
| 295 | AI551472_ESTs | 1 | stomach | panc, liv | −1.3 | 1.3 | 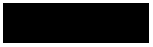 | 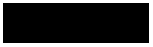 |
| 113 | AA546645_cell_division_cycle_2_homolog_(s._pombe)- like_2 | 1 | colon | panc, liv, col, brain, pl/ut | −1.4 | [symbol] | 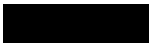 | 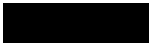 |
| 194 | 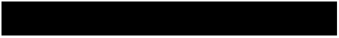 | 1 | liver | panc, liv, int | −1.8 | [symbol] | 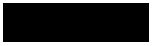 | 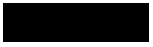 |
| 293 | AA497627_intersectin_(SH3_domain_protein_1A) | 1 | stomach | panc, liv, lung, brain | −1.1 | 1.4 | 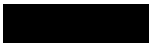 | 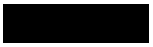 |
| 76 | 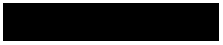 | 1 | bladder | panc, liv, stom, col, brain, blad | 1.1+ | 1+ | 1.4+ | 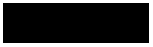 |
| 88 | AI122516_microtubule-associated_protein_6 | 1 | brain | panc, lung, brain | −1.1 | −1.2 | 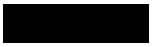 | 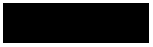 |
| 31 | AA638765_metallothionein_1 | 2 | liver, intestine | panc, pl/ut | −3.5 | −3.6 | 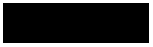 | 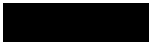 |
| 218 | W36474_metallothionein_2 | 1 | liver | panc, pl/ut | −3.2 | −2.7 | 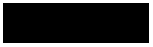 | 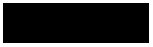 |
| 268 | AA600634_signal_sequence_receptor,_delta | 1 | pancreas | panc, stom | −1.3 | −1.8 | 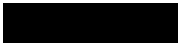 | 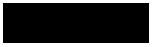 |
| 48 | 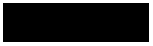 | 2 | stomach; brain | panc, stom | −1.3 | −1.3 | 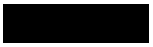 | 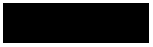 |
| 264 | AA790398_ribonuclease_I,_pancreatic | 1 | pancreas | panc, stom | −1.6 | −7.7 | 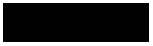 | 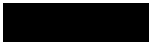 |
| 9 | AA024217_Public_domain_EST | 3 | liver; intestine; colon | panc, stom, col | −4.1* | −1.7 | 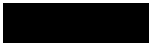 | 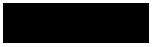 |
| 87 | 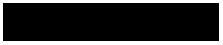 | 1 | brain | panc, stom | −1.2 | 1.2 | 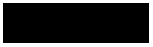 | 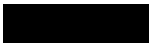 |
| 92 | 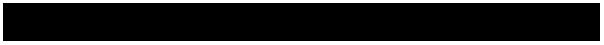 | 1 | brain | panc, stom, pl/ut | −1.3 | 1.1 | 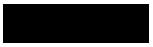 | 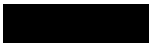 |
| 336 | 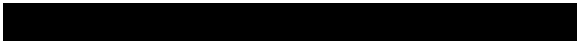 | 1 | stomach | panc? | 1 | −1.1 | 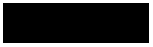 | 1.5 |
| 442 | 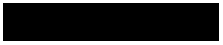 | 1 | stomach | panc? | 1.3 | −1.1+ | 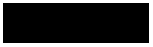 | 1.6+ |
| 356 | 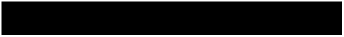 | 1 | stomach | panc? | 1.1 | 1 | 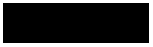 | 1.5 |
| 384 | 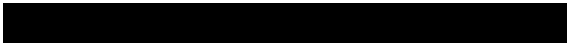 | 1 | stomach | panc? | −1.1 | −1.3 | 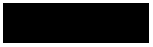 | 1.5 |
| 408 | 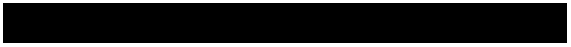 | 1 | stomach | panc? | 1.2 | −1.1 | 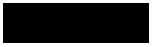 | [symbol] |
| 396 | 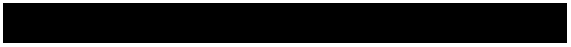 | 1 | stomach | panc? | −1.2 | −1.6 | 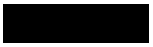 | 1.6 |
| 137 | AA067236_fibrlblast_growth_factor_receptor_1 | 1 | colon | pl/ut | −2.5 | −2.1 | −1.1 | −1.1 |
| 119 | AA571365_Mus_musculus_MRPS6_mRNA_for_mito- | 1 | colon | pl/ut | −1.8 | −1.5 | −1.3 | −1.2 |
| | chondrial_ribosomal_protein_S6,_partial_cds | | | | | | | |
| 179 | AA013726_cathepsin_1 | 1 | intestine | [symbol] | −3.1 | −1.7 | −1.6 | |
| 255 | 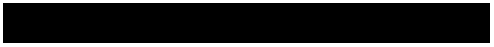 | 1 | mammary gland | pl/ut | −16 | −23 | −14 | −7 |
| 61 | AA717167_cytotoxic_T_lymphocyte- | 2 | stomach; colon | pl/ut | −2.5 | −3.5 | −3.4 | −1.9 |
| | associated_protein_2_alpha | | | | | | | |
| 63 | AA870247_mitogen_regulated_protein,_proliferin_3 | 2 | stomach; intestine | pl/ut | −3.8 | −3.7 | −1.5 | −1.6 |
| 190 | AI155437_Mus_musculus_20alpha- | 1 | kidney | pl/ut, breast | −1.2 | −1.5 | −1.1 | 1.3 |
| | HSD_mRNA_for_20alpha- | | | | | | | |
| | hydroxysteroid_dehydrogenase,_complete_cds | 1 | stomach | | | | | |
| 298 | AI597531_gap_junction_membrane_channel_protein_beta_2 | −1.9 | −2.3 | 1.1 | −1.3 | | | |
| 81 | 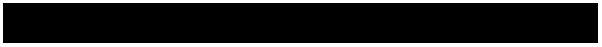 | 1 | brain | spl | 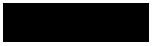 | 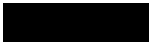 | −6.4 | −4.7* |
| 82 | 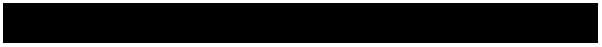 | 1 | brain | spl | 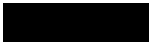 | 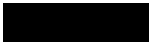 | −6.8 | −5.6 |
| 83 | 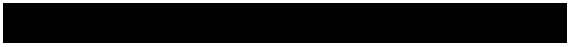 | 1 | brain | spl | 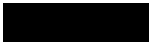 | 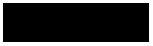 | −5.7 | −4.9 |
| 85 | 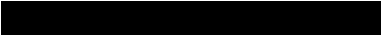 | 1 | brain | spl | 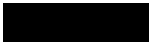 | 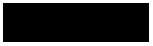 | −2.5 | −2.8 |
| 96 | 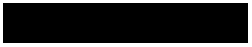 | 1 | brain | spl | 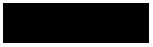 | 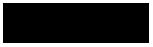 | −2.5 | −1.8 |
| 84 | W14953_kinesin-associated_protein_3 | 1 | brain | spl | 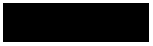 | 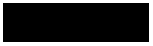 | −3.5 | −1.3 |
| 20 | AI324398_glycosylphosphatidylinositol_specific_phospholipase_D1 | 2 | intestine; brain | spl | 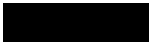 | 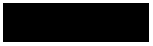 | −6 | −4.5 |
| 150 | AA166427_Mus_musculus_germline_immunoglobulin_gam- ma_constant_region_(IgG3)_mRNA | 1 | intestine | spl | 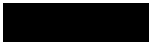 | 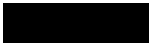 | −1.6 | −1.7 |
| 208 | AA771355_ficolin_A | 1 | liver | spl | 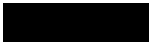 | 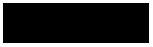 | −2 | −1.2 |
| 97 | 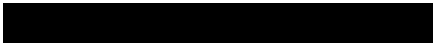 | 1 | brain | spl, brain | 1.3 | 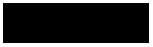 | 1.3 | 1.1 |
| 243 | AA145895_heat_shock_protein,_70_kDa_1 | 1 | lung | spl, int, col | 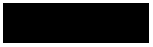 | 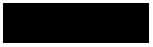 | −1.7 | −1.5 |
| 176 | AA239419_P_glycoprotein_2 | 1 | intestine | spl, liv, col, pl/ut | 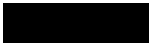 | 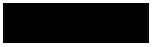 | −2.9 | −2.4 |
| 363 | AA254235_complement_receptor_2 | 1 | stomach | spl, panc | 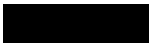 | 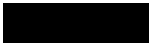 | 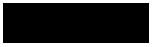 | 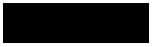 |
| 77 | AA612413_chiatinase_acidic | 1 | bladder | stom | −37 | −35 | −27 | −21 |
| 114 | AI429054_ESTs | 1 | colon | stom | 1.3 | 1.3 | 1.2 | 1.4 |
| 158 | AA684403_ESTs | 1 | intestine | stom | −2.4 | −1.7 | 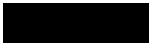 | 1.2 |
| 191 | AA667301_troporin_I,_cardiac | 1 | kidney | stom | [symbol] | −9 | −5.2 | [symbol] |
| 271 | AA210481_clusterin | 1 | pancreas | stom | −1.6 | −1.7 | 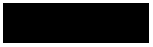 | −1.6 |
| 266 | AI120322_RIKEN_cDNA_1810055P16_gene | 1 | pancreas | stom | −1.1 | −1.3 | 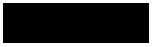 | −1.2 |
| 267 | AA763351_death-associated_kinase_3 | 1 | pancreas | stom | −1.1 | −1.3 | 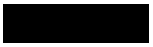 | −1.1 |
| 261 | 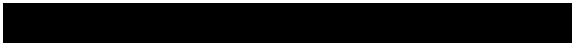 | 1 | pancreas | stom, breast | 1.3 | 1.1 | 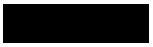 | 1.5 |
| 95 | AA119984_liver-specific_bHLH-Zip_transcription_factor | 1 | brain | stom, int | −1.6 | −2.3 | 1 | −1.2 |
| 144 | AI892747_glutathione_S-transferase,_alpha_1_(Ya) | 1 | colon | stom, int | −5.4 | −4.7 | −3.7 | −2.8 |
| 133 | AA606610_RIKEN_cDNA_2510002C21_gene | 1 | colon | stom, int, blad | −3 | −3.4 | −2.3 | −2.2 |
| 23 | AI385503_cysteine_rich_intestinal_protein | 2 | intestine; colon | stom, int, col | −1.6 | −2 | −1.1 | −1.3 |
| 159 | AA210237_Mus_musculus,_clone_MGC6377,_mRNA_com- | 1 | intestine | stom, int, col | 1.4 | 1.6 | −1.2 | −1.2 |
| | plete_cds | | | | | | | |
| 188 | W16059_glutathione_S-transferase_omega_1 | 1 | kidney | stom, int, col | −2.3 | −2.7 | −2.2 | −2 |
| 260 | W08923_cytochrome_c_oxidase_subunit_VI_a,_polypeptide_1 | 1 | pancreas | stom, int, col, | −1.8* | −1.4* | 1*** | −2.7 |
| | | | | kidn | | | | |
| 245 | AA002910_FBJ_osteosarcoma_oncogene | 1 | lung | stom, int, col, | −1.1 | −1.1 | −1.2 | 1.1 |
| | | | | lung, brain | | | | |
| 78 | W82228_annexin_A3 | 1 | bladder | stom, lung, | 1 | −1.5 | −1.5 | −1.1 |
| | | | | pl/ut | | | | |
| 123 | AA437876_degenerative_spermatocyte_homolog_(Drosophila) | 1 | colon | stom, pl/ut | −1.2 | −1.4 | −1.4 | −1.7 |
| 91 | 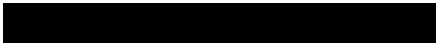 | 1 | colon | | | | | |
| 99 | 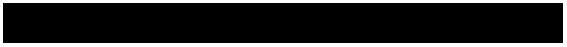 | 1 | brain | | | | | |
| 101 | 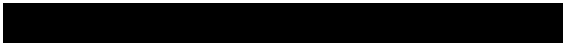 | 1 | brain | | | | | |
| 109 | AA727967_serine_(or_cysteine)_proteinase_in- | 1 | colon | | | | | |
| | hibitor,_clade_F_(alpha-2_amtiplasmin,_pigment_epithe- | | | | | | | |
| | lium_derived_factor)_member_1 | | | | | | | |
| 125 | AA725966_shingosine_kinase_I | 1 | colin | | | | | |
| 130 | AA062408_ESTs,_Moderately_similar_to_proteasome_in- | 1 | colin | | | | | |
| | hibitor_hP131_subunit_[H.sapiens] | | | | | | | |
| 131 | AA619789_methylenetetrahydrofolate_dehydrogenase_(NAD+_de- | 1 | colin | | | | | |
| | pendent),_methenyltetrahydrofolate_cyclohydrolase | | | | | | | |
| 138 | AA619953_ESTs,_Weakly_similar_to_T25027_hypo- | 1 | colin | | | | | |
| | thetical_protein_T20S3.5_-_Caenorhabditis_elegans_[C.elegans] | | | | | | | |
| 140 | AI595630_RIKEN_cDNA_4930578I06_gene | 1 | colin | | | | | |
| 142 | AA106834_ESTs,_Weakly_similar_to_EP15_MOUSE_EPI- | 1 | colin | | | | | |
| | DERMAL_GROWTH_FACTOR_RECEPTOR_SUB- | | | | | | | |
| | STRATE_SUBSTRATE_15_[M.musculus] | | | | | | | |
| 145 | AA871395_Mus_musculus,_Simi- | 1 | colin | | | | | |
| | lar_to_ectonucleotide_pyrophosphatase/ | | | | | | | |
| | phosphodiesterase_3._clone_IMAGE:3495326,_mRNA,_partial_cds | | | | | | | |
| 162 | AA109528_ESTs,_Weakly_simi- | 1 | intestine | | | | | |
| | lar_to_CAIC_MOUSE_COLLAGEN_AL- | | | | | | | |
| | PHA_1(XII)_CHAIN_PRECURSOR_[M.musculus] | | | | | | | |
| 169 | AI552688_ESTs,_Weakly_simi- | 1 | intestine | | | | | |
| | lar_to_MIA_MOUSE_MELA- | | | | | | | |
| | NOMA_DERIVED_GROWTH_REGULATORY_PRO- | | | | | | | |
| | TEIN_PRECURSOR_[M.musculus] | | | | | | | |
| 173 | AA823657_UDP-N-acetyl-alpha-D-galactosamine:(N- | 1 | intestine | | | | | |
| | acetylneuraminyl)-galactosyl-N- | | | | | | | |
| | acetylglucosaminylpolypeptide-beta-1,_4-N- | | | | | | | |
| | acetylgalactosaminyltransferase | | | | | | | |
| 210 | AA420359_DNA_segment,_Chr_6,_ERATO_Coi_109,_expressed | 1 | liver | | | | | |
| 236 | AA543982_interleukin_1_receptor,_type_1 | 1 | liver | | | | | |
| 238 | AI323261_lipase hepatic | 1 | liver | | | | | |
| 239 | AI425345_hydroxysteroid_17-beta_dehydrogenase_9 | 1 | liver | | | | | |
| 240 | AA274023_apoptosis_inhibitory_6 | 1 | liver | | | | | |
| 242 | AA067863_RIKEN_cDNA_2310057I18_gene | 1 | lung | | | | | |
| 246 | AA657278_Dnal_(Hsp40)_homolog,_subfamily_B,_member_1 | 1 | lung | | | | | |
| 276 | AA458425_Swi/SNF_related_matrix_associted,_actin_de- | 1 | panc | | | | | |
| | pendent_regulator_of_chromatin,_sub- | | | | | | | |
| | family_a-like_1 | | | | | | | |
| 279 | AA5179006_T-box_15 | 1 | placenta/uterus | | | | | |
| 40 | AA717025_Mus_musculus_10_day_old_male_pan- | 2 | spleen; brain | | | | | |
| | creas_cDNA_RIKEN_full- | | | | | | | |
| | length_enriched_library,_clone:1810009A17,_full_insert_se- | | | | | | | |
| | quence | | | | | | | |
| 43 | AA822106_ESTs,_Weakly_similar_to_CO8A_HUMAN_COM- | 2 | spleen; liver | | | | | |
| | PLEMENT_COMPLEMENT_COMPONENT_C8_AL- | | | | | | | |
| | PHA_CHAIN_PRECURSOR_[H.sapiens] | | | | | | | |
| 6 | AI447967_ESTs | 4 | stomach; brain; placenta/uterus; | | | | | |
| | | | mammary gland | | | | | |
| 53 | AI614738_RIKEN_cDNA_2610301I15_gene | 2 | stomach; brain | | | | | |
| 55 | AI551391_ESTs,_Weakly_simi- | 2 | stomach; brain | | | | | |
| | lar_to_FVI_MOUSE_FRIEND_VIRUS_SUS- | | | | | | | |
| | CEPTIBILITY_PROTEIN_1_[M.musculus] | | | | | | | |
| 56 | AA473684_RIKEN_cDNA_C030002O17_gene | 2 | stomach; brain | | | | | |
| 57 | AI613926_twist_gene_homolog_(Drosophila) | 2 | stomach; colon | | | | | |
| 65 | AI549666_ESTs | 2 | stomach; kidney | | | | | |
| 3 | AI553220_ESTs | 5 | stomach; lung; bladder; | | | | | |
| | | | placenta/uterus; mammary gland | | | | | |
| 69 | AI549654_programmed_cell_death_10 | 2 | stomach; mammary gland | | | | | |
| 73 | AA173562_ESTs,_Moderately_simi- | 2 | stomach; placenta/uterus | | | | | |
| | lar_to_alias_DLC1_[H.sapiens] | | | | | | | |
| 294 | AA655910_mast_cell_protease_2 | 1 | stomach | | | | | |
| 297 | AI552048_ESTs | 1 | stomach | | | | | |
| 299 | AA450831_BC1.2/adenovirus_E1B_19_kDa- | 1 | stomach | | | | | |
| | interacting_protein_1,_NIP2 | | | | | | | |
| 300 | AA517193_unc119_homolog_[C._elegans) | 1 | stomach | | | | | |
| 307 | AA211954_RIKEN_cDNA_4930511A05_gene | 1 | stomach | | | | | |
| 308 | AA530026_ESTs | 1 | stomach | | | | | |
| 311 | AA438040_Public_domain_EST | 1 | stomach | | | | | |
| 317 | AA470294_DNA_segment,_Chr_2,_ERATO_Doi_435,_ex- | 1 | stomach | | | | | |
| | pressed | | | | | | | |
| 320 | AA265406_nuclear_factor_I/B | 1 | stomach | | | | | |
| 325 | AA210559_ESTs,_Weakly_similar_to_T34029_hypo- | 1 | stomach | | | | | |
| | thetical_protein_C32F10.1_-_Caenorhabditis_elegans_[C.elegans] | | | | | | | |
| 327 | AI553356_ESTs,_Moderately_similar_to_T00075_hypo- | 1 | stomach | | | | | |
| | thetical_protein_KIAA0461_[H.sapiens] | | | | | | | |
| 330 | AA254875_ESTs | 1 | stomach | | | | | |
| 339 | AA596525_chromodomain_helicase_DNA_binding_protein_1 | 1 | stomach | | | | | |
| 341 | 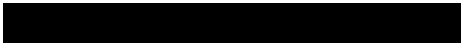 | 1 | stomach | | | | | |
| 345 | 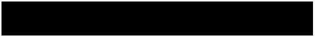 | 1 | stomach | | | | | |
| 348 | 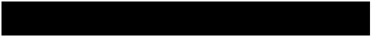 | 1 | stomach | | | | | |
| 349 | AA438136_nuclear_localization_signal_protein_absent_in_velo- | 1 | stomach | | | | | |
| | cardio-facial_patients | | | | | | | |
| 352 | 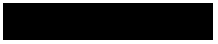 | 1 | stomach | | | | | |
| 360 | 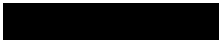 | 1 | stomach | | | | | |
| 364 | 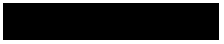 | 1 | stomach | | | | | |
| 373 | 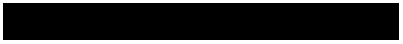 | 1 | stomach | | | | | |
| 375 | 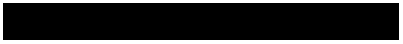 | 1 | stomach | | | | | |
| 378 | 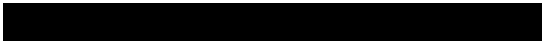 | 1 | stomach | | | | | |
| 381 | 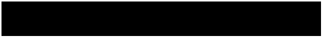 | 1 | stomach | | | | | |
| 385 | 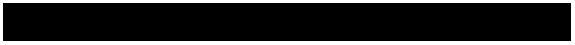 | 1 | stomach | | | | | |
| 395 | 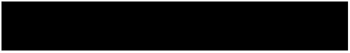 | 1 | stomach | | | | | |
| 397 | 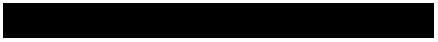 | 1 | stomach | | | | | |
| 411 | 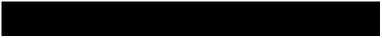 | 1 | stomach | | | | | |
| 413 | 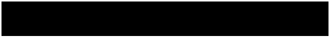 | 1 | stomach | | | | | |
| 416 | 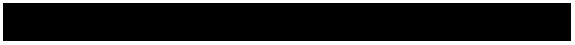 | 1 | stomach | | | | | |
| 428 | 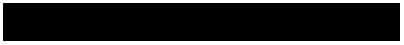 | 1 | stomach | | | | | |
| 437 | 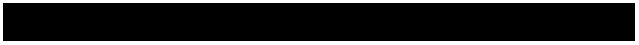 | 1 | stomach | | | | | |
| 438 | 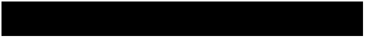 | 1 | stomach | | | | | |
| 440 | 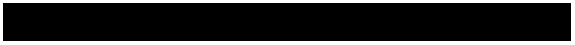 | 1 | stomach | | | | | |
| 9 | 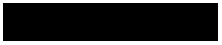 | 1 | stomach | | | | | |
| 443 | 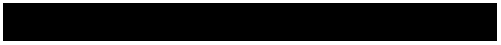 | 1 | stomach | | | | | |
| 445 | 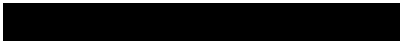 | 1 | stomach | | | | | |

| | | 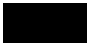 | | 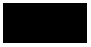 | | 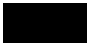 | | 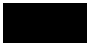 | |
|---|---|---|---|---|---|---|---|---|---|
| | | (−) | (+) | (−) | (+) | (−) | (+) | (−) | (+) |
| IDI | Description | PL-6B_BDE | PL-18B_BDE | PL-8B_BDE | PL-20B_BDE | PL-3B_BDE | PL-15B_BDE | PL-5B_BDE | PL-17B_BDE |
| 225 | AA116636_RIKEN_cDNA_1110013A05_gene | 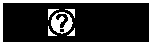 | 1.3 | 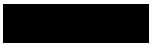 | 1.3 | 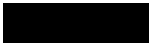 | 1.4 | 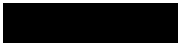 | 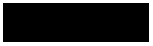 |
| 284 | AA237793_rat_regenerating_islet- derived,_mouse_homolog_1 | −15 | −12 | −3.2 | 1.2 | −2.2 | 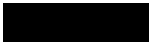 | −12 | −5.6 |
| 28 | AA242201_serine_protease_inhibitor,_Kazal_type_3 | −5.3 | −3.8 | −1.9 | −2.2 | 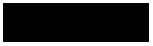 | 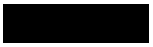 | 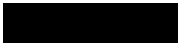 | 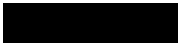 |
| 201 | AA867621_S-phase_kinase-associated_protein_2_(p45) | 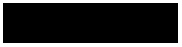 | 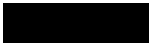 | 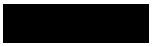 | 1.2 | 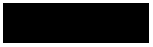 | 1.5 | 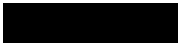 | 1.4+* |
| 232 | AI893740_glycine_N-methyltransferase | 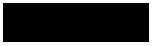 | 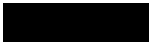 | 1.4 | 1.3 | 1 | −1.2 | −1.2 | −1.7* |
| 353 | AA684073_ESTs | 1.2 | 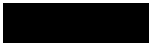 | 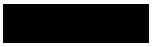 | 1.1 | 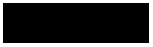 | 1.1 | 1.3 | 1.2+* |
| 295 | AI551472_ESTs | 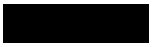 | 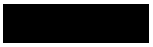 | 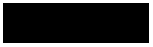 | 1 | 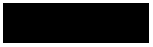 | 1.5 | 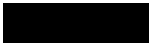 | [symbol] |
| 113 | AA546645_cell_division_cycle_2_homolog_(s._pombe)- like_2 | 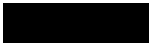 | 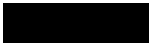 | 1.5 | 1.1 | 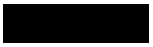 | [symbol] | 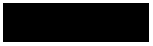 | 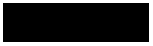 |
| 194 | 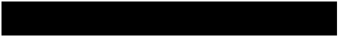 | 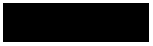 | 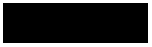 | −1.1 | −1.2 | 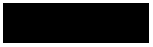 | 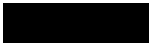 | 1.4 | −1.5 |
| 293 | AA497627_intersectin_(SH3_domain_protein_1A) | 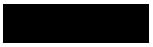 | 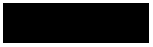 | 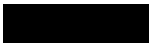 | −1.1 | 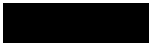 | 1.3 | 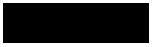 | 1.1+ |
| 76 | 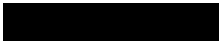 | 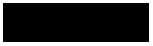 | 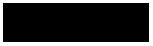 | 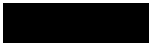 | 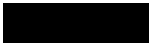 | 1.2 | 1.4+ | 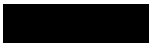 | 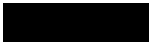 |
| 88 | AI122516_microtubule-associated_protein_6 | −1.2 | 1.4 | 1.6 | 1.2 | 1.3 | −1.1 | 1 | −1.3+* |
| 31 | AA638765_metallothionein_1 | 1.3 | −1.9 | 1.4 | 1.1 | 1.3 | −1.9 | −1.4 | −2.3 |
| 218 | W36474_metallothionein_2 | '11.3 | 2.8 | 1 | −1.1 | −1.7 | −3.2 | −1.9 | −3.3 |
| 268 | AA600634_signal_sequence_receptor,_delta | 1 | −1.1 | 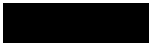 | 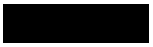 | −1 | 1 | 1.1 | 1.3 |
| 48 | 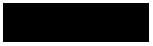 | −1.1 | 1.2 | 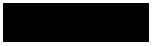 | 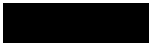 | 1.4 | 1.6 | −1 | 1.3+ |
| 264 | AA790398_ribonuclease_I,_pancreatic | −21 | −18 | −3.2 | 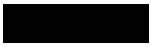 | −20 | −1.2 | −15 | −4.5 |
| 9 | AA024217_Public_domain_EST | 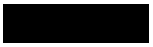 | 1.4 | 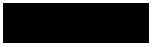 | 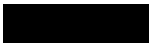 | 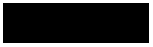 | 1 | 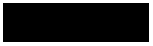 | 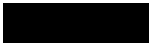 |
| 87 | 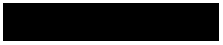 | 1.1 | 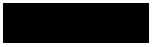 | 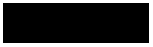 | 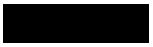 | 1.6 | 1.3 | 1.1 | 1.1+* |
| 92 | 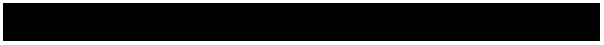 | 1.1 | 1.6 | 1.4 | 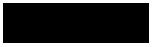 | 1.3 | 1.2 | 1.3 | 1.4+* |
| 336 | 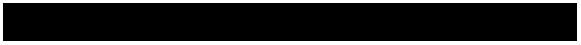 | −1.1 | 1.2 | 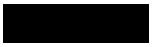 | −1.1 | 1.5 | 1 | 1.1 | −1 |
| 442 | 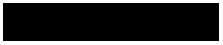 | 1.1+ | 1.1 | 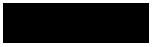 | '11 | 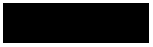 | 1.1+ | 1.2+ | 1.2+ |
| 356 | 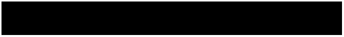 | 1 | 1.2 | 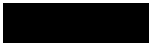 | −1 | 1.5 | 1.1 | −1.1 | 1.1+* |
| 384 | 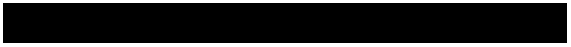 | −1.1 | 1.2 | 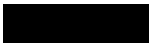 | −1.2 | 1.3 | −1.3 | −1.1 | −1.7+* |
| 408 | 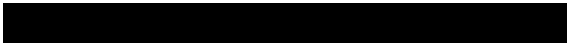 | 1 | 1.1 | 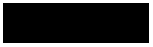 | −1.3 | 1.4 | −1 | −1.1 | −1.2 |
| 396 | 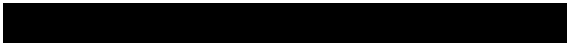 | −1.2 | 1.1 | 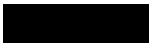 | −1.5 | 1.2 | −1.2 | −1.3 | −1.1 |
| 137 | AA067236_fibrlblast_growth_factor_receptor_1 | −1.1 | −1.5 | −1.1 | −2 | −1.2 | −1.8 | 1.1 | −1.8 |
| 119 | AA571365_Mus_musculus_MRPS6_mRNA_for_mito- chondrial_ribosomal_protein_S6,_partial_cds | 1.1 | −1.2 | −1.1 | −1.4 | −1 | −1.3 | 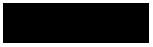 | −1.4* |
| 179 | AA013726_cathepsin_1 | −1.8 | −1.5 | 1.1 | −1.7 | −1.2 | −2.3 | −1.5 | −1.1 |
| 255 | 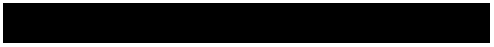 | −11 | −9.7 | −6.6 | −9.8 | −9.4 | −10 | −9.9 | −9.5 |
| 61 | AA717167_cytotoxic_T_lymphocyte- | −2.7 | −2.1 | −1.4 | −2.9 | −2.4 | −3.4 | −2.1 | −4.3 |
| | associated_protein_2_alpha | | | | | | | | |
| 63 | AA870247_mitogen_regulated_protein,_proliferin_3 | −2.3 | −1.8 | −1.2 | −2.3 | −1.4 | −2.7 | −2 | −2 |
| 190 | AI155437_Mus_musculus_20alpha- | −1.2 | 1 | 1.2 | 1 | −1 | −1.3 | −1.2 | −1.3+ |
| | HSD_mRNA_for_20alpha- | | | | | | | | |
| | hydroxysteroid_dehydrogenase,_complete_cds | | | | | | | | |
| 298 | AI597531_gap_junction_membrane_channel_protein_beta_2 | 1.1 | 1.2 | −1.1 | −2.2 | [symbol] | −1.9 | −1.8 | −2.1+ |
| 81 | 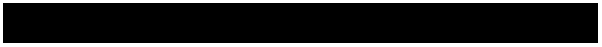 | −5.6 | −6.8 | −11 | −3.6 | −20 | −1.9 | −14 | −3.4 |
| 82 | 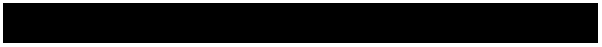 | −5.6 | −7.7 | −1.1 | −2.7 | −24 | −25 | −14 | −3.3 |
| 83 | 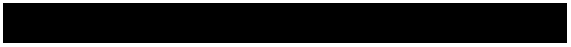 | −5.8 | −6.9 | −7.4 | −2.6 | −15 | −21 | −13 | −3.4 |
| 85 | 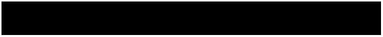 | −3.5 | −3.9 | −4.2 | −2.7 | −3.8 | −4.4 | −4.7 | −1.8 |
| 96 | 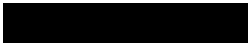 | −1.3 | −1.4 | −2.4 | −2.9 | −2.3 | −1.3 | −1.3 | −2.3* |
| 84 | W14953_kinesin-associated_protein_3 | −3.3 | −4.6 | −6.5 | −2.1 | −9.4 | −7.9 | −8 | −2 |
| 20 | AI324398_glycosylphosphatidylinositol_specific_phospholipase_D1 | −2.2 | −2.7 | −5.8 | −1.7 | −2.4 | −5.6 | −1.5 | 1.1 |
| 150 | AA166427_Mus_musculus_germline_immunoglobulin_gam- | −2 | −2 | −1.8 | −1.9 | 1.4 | −1.5 | −1.2 | 1 |
| | ma_constant_region_(IgG3)_mRNA | | | | | | | | |
| 208 | AA771355_ficolin_A | 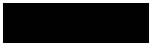 | 1.2 | −1.6* | −2.1 | −2.3 | −2.7 | −1.6 | −1.6 |
| 97 | 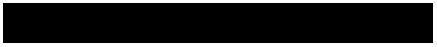 | −1.3 | 1.2 | 1.2 | −1.1 | 1.1 | −1.1 | −1.2 | −1+ |
| 243 | AA145895_heat_shock_protein,_70_kDa_1 | −1.4 | −1.4 | 1.2 | −1 | 1.5 | 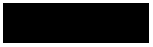 | 1.1 | 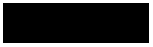 |
| 176 | AA239419_P_glycoprotein_2 | 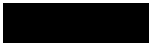 | 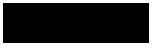 | −1.9 | −1.8 | 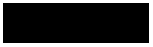 | 1.3 | 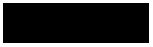 | 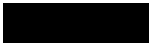 |
| 363 | AA254235_complement_receptor_2 | 1 | 1.4 | 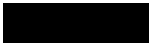 | −1.1 | 1.4 | −1 | 1.2 | 1.2 |
| 77 | AA612413_chiatinase_acidic | −13 | −24 | 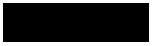 | 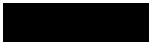 | −24 | −28 | −12 | −38 |
| 114 | AI429054_ESTs | 1.6 | 1.3 | 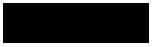 | 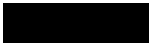 | 1.2 | 1.4 | 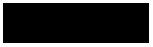 | 1.2 |
| 158 | AA684403_ESTs | −2.1 | 1.1 | 1.3 | 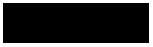 | 1.3 | [symbol] | −1.5 | 1.6* |
| 191 | AA667301_troporin_I,_cardiac | −5.2 | −1.9 | −1.2 | 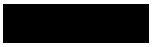 | −5.7 | −7.2 | −3.5 | −4.9 |
| 271 | AA210481_clusterin | 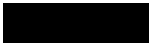 | 1.6 | −1.1 | 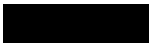 | −5.2 | −2.8 | −5.6 | −4.1 |
| 266 | AI120322_RIKEN_cDNA_1810055P16_gene | −2.9 | −2.3 | 1.5 | 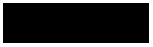 | −1.3 | −1.4 | −1.4 | 1.2 |
| 267 | AA763351_death-associated_kinase_3 | −2.6 | −2.1 | 1.5 | 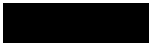 | −1.3 | −1.3 | −1.3 | 1.2 |
| 261 | 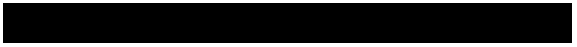 | −1.2 | −1.1 | 1.5 | 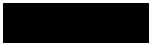 | 1.1 | −1 | 1.1 | 1.2 |
| 95 | AA119984_liver-specific_bHLH-Zip_transcription_factor | 1.6 | 1.5 | 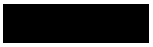 | 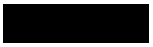 | 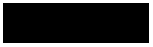 | 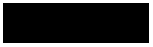 | 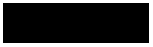 | 1.4 |
| 144 | AI892747_glutathione_S-transferase,_alpha_1_(Ya) | [symbol] | −1 | 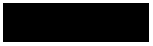 | 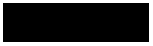 | 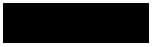 | 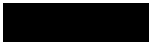 | 1 | −2 |
| 133 | AA606610_RIKEN_cDNA_2510002C21_gene | −2.5 | −2.6 | 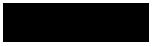 | 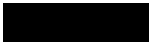 | 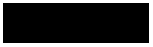 | 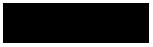 | 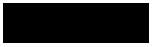 | −1.2 |
| 23 | AI385503_cysteine_rich_intestinal_protein | −2.3 | −2.2 | 1.5 | 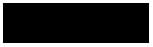 | 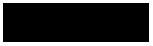 | 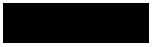 | 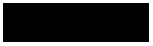 | 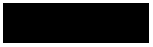 |
| 159 | AA210237_Mus_musculus,_clone_MGC6377,_mRNA_com- plete_cds | −1.9 | −1.9 | 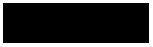 | 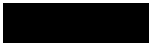 | 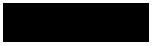 | 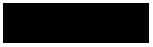 | 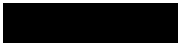 | 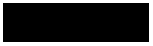 |
| 188 | W16059_glutathione_S-transferase_omega_1 | 1.3 | −1.3 | 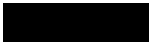 | 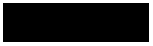 | 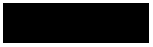 | 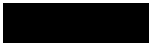 | 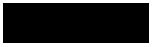 | 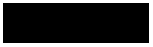 |
| 260 | W08923_cytochrome_c_oxidase_subunit_VI_a,_polypeptide_1 | 1 | 1 | 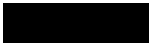 | 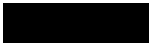 | 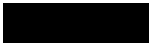 | 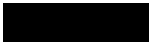 | [symbol] | 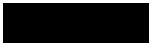 |
| 245 | AA002910_FBJ_osteosarcoma_oncogene | −1.3 | 1.1 | 1.4 | 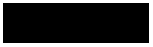 | 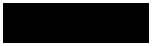 | 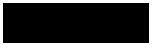 | 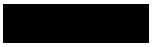 | 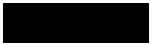 |
| 78 | W82228_annexin_A3 | −1.4 | −1.5 | 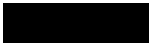 | 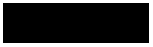 | −1.9 | −1.1 | −1.2 | 1.5 |
| 123 | AA437876_degenerative_spermatocyte_homolog_(Drosophila) | 1.1 | −1.3 | 1.1 | 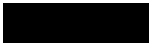 | −1.3 | −1.6 | −1.1 | −2.3 |
| 91 | 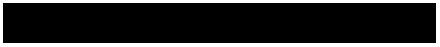 | | | | | | | | |
| 99 | 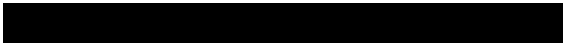 | | | | | | | | |
| 101 | 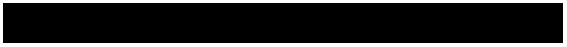 | | | | | | | | |
| 109 | AA727967_serine_(or_cysteine)_proteinase_in- | | | | | | | | |
| | hibitor,_clade_F_(alpha-2_amtiplasmin,_pigment_epithe- | | | | | | | | |
| | lium_derived_factor)_member_1 | | | | | | | | |
| 125 | AA725966_shingosine_kinase_I | | | | | | | | |
| 130 | AA062408_ESTs,_Moderately_similar_to_proteasome_in- | | | | | | | | |
| | hibitor_hP131_subunit_[H.sapiens] | | | | | | | | |
| 131 | AA619789_methylenetetrahydrofolate_dehydrogenase_(NAD+_de- | | | | | | | | |
| | pendent),_methenyltetrahydrofolate_cyclohydrolase | | | | | | | | |
| 138 | AA619953_ESTs,_Weakly_similar_to_T25027_hypo- | | | | | | | | |
| | thetical_protein_T20S3.5_-_Caenorhabditis_elegans_[C.elegans] | | | | | | | | |
| 140 | AI595630_RIKEN_cDNA_4930578I06_gene | | | | | | | | |
| 142 | AA106834_ESTs,_Weakly_similar_to_EP15_MOUSE_EPI- | | | | | | | | |
| | DERMAL_GROWTH_FACTOR_RECEPTOR_SUB- | | | | | | | | |
| | STRATE_SUBSTRATE_15_[M.musculus] | | | | | | | | |
| 145 | AA871395_Mus_musculus,_Simi- | | | | | | | | |
| | lar_to_ectonucleotide_pyrophosphatase/ | | | | | | | | |
| | phosphodiesterase_3._clone_IMAGE:3495326,_mRNA,_partial_cds | | | | | | | | |
| 162 | AA109528_ESTs,_Weakly_simi- | | | | | | | | |
| | lar_to_CAIC_MOUSE_COLLAGEN_AL- | | | | | | | | |
| | PHA_1(XII)_CHAIN_PRECURSOR_[M.musculus] | | | | | | | | |
| 169 | AI552688_ESTs,_Weakly_simi- | | | | | | | | |
| | lar_to_MIA_MOUSE_MELA- | | | | | | | | |
| | NOMA_DERIVED_GROWTH_REGULATORY_PRO- | | | | | | | | |
| | TEIN_PRECURSOR_[M.musculus] | | | | | | | | |
| 173 | AA823657_UDP-N-acetyl-alpha-D-galactosamine:(N- | | | | | | | | |
| | acetylneuraminyl)-galactosyl-N- | | | | | | | | |
| | acetylglucosaminylpolypeptide-beta-1,_4-N- | | | | | | | | |
| | acetylgalactosaminyltransferase | | | | | | | | |
| 210 | AA420359_DNA_segment,_Chr_6,_ERATO_Coi_109,_expressed | | | | | | | | |
| 236 | AA543982_interleukin_1_receptor,_type_1 | | | | | | | | |
| 238 | AI323261_lipase hepatic | | | | | | | | |
| 239 | AI425345_hydroxysteroid_17-beta_dehydrogenase_9 | | | | | | | | |
| 240 | AA274023_apoptosis_inhibitory_6 | | | | | | | | |
| 242 | AA067863_RIKEN_cDNA_2310057I18_gene | | | | | | | | |
| 246 | AA657278_Dnal_(Hsp40)_homolog,_subfamily_B,_member_1 | | | | | | | | |
| 276 | AA458425_Swi/SNF_related_matrix_associted,_actin_de- | | | | | | | | |
| | pendent_regulator_of_chromatin,_sub- | | | | | | | | |
| | family_a-like_1 | | | | | | | | |
| 279 | AA5179006_T-box_15 | | | | | | | | |
| 40 | AA717025_Mus_musculus_10_day_old_male_pan- | | | | | | | | |
| | creas_cDNA_RIKEN_full- | | | | | | | | |
| | length_enriched_library,_clone:1810009A17,_full_insert_se- | | | | | | | | |
| | quence | | | | | | | | |
| 43 | AA822106_ESTs,_Weakly_similar_to_CO8A_HUMAN_COM- | | | | | | | | |
| | PLEMENT_COMPLEMENT_COMPONENT_C8_AL- | | | | | | | | |
| | PHA_CHAIN_PRECURSOR_[H.sapiens] | | | | | | | | |
| 6 | AI447967_ESTs | | | | | | | | |
| 53 | AI614738_RIKEN_cDNA_2610301I15_gene | | | | | | | | |
| 55 | AI551391_ESTs,_Weakly_simi- | | | | | | | | |
| | lar_to_FVI_MOUSE_FRIEND_VIRUS_SUS- | | | | | | | | |
| | CEPTIBILITY_PROTEIN_1_[M.musculus] | | | | | | | | |
| 56 | AA473684_RIKEN_cDNA_C030002O17_gene | | | | | | | | |
| 57 | AI613926_twist_gene_homolog_(Drosophila) | | | | | | | | |
| 65 | AI549666_ESTs | | | | | | | | |
| 3 | AI553220_ESTs | | | | | | | | |
| 69 | AI549654_programmed_cell_death_10 | | | | | | | | |
| 73 | AA173562_ESTs,_Moderately_simi- | | | | | | | | |
| | lar_to_alias_DLC1_[H.sapiens] | | | | | | | | |
| 294 | AA655910_mast_cell_protease_2 | | | | | | | | |
| 297 | AI552048_ESTs | | | | | | | | |
| 299 | AA450831_BC1.2/adenovirus_E1B_19_kDa- | | | | | | | | |
| | interacting_protein_1,_NIP2 | | | | | | | | |
| 300 | AA517193_unc119_homolog_[C._elegans) | | | | | | | | |
| 307 | AA211954_RIKEN_cDNA_4930511A05_gene | | | | | | | | |
| 308 | AA530026_ESTs | | | | | | | | |
| 311 | AA438040_Public_domain_EST | | | | | | | | |
| 317 | AA470294_DNA_segment,_Chr_2,_ERATO_Doi_435,_ex- | | | | | | | | |
| | pressed | | | | | | | | |
| 320 | AA265406_nuclear_factor_I/B | | | | | | | | |
| 325 | AA210559_ESTs,_Weakly_similar_to_T34029_hypo- | | | | | | | | |
| | thetical_protein_C32F10.1_-_Caenorhabditis_elegans_[C.elegans] | | | | | | | | |
| 327 | AI553356_ESTs,_Moderately_similar_to_T00075_hypo- | | | | | | | | |
| | thetical_protein_KIAA0461_[H.sapiens] | | | | | | | | |
| 330 | AA254875_ESTs | | | | | | | | |
| 339 | AA596525_chromodomain_helicase_DNA_binding_protein_1 | | | | | | | | |
| 341 | 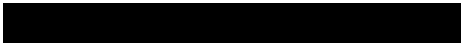 | | | | | | | | |
| 345 | 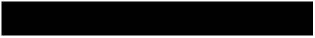 | | | | | | | | |
| 348 | 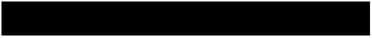 | | | | | | | | |
| 349 | AA438136_nuclear_localization_signal_protein_absent_in_velo- | | | | | | | | |
| | cardio-facial_patients | | | | | | | | |
| 352 | 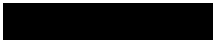 | | | | | | | | |
| 360 | 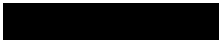 | | | | | | | | |
| 364 | 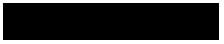 | | | | | | | | |
| 373 | 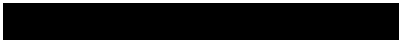 | | | | | | | | |
| 375 | 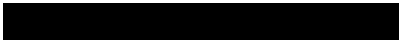 | | | | | | | | |
| 378 | 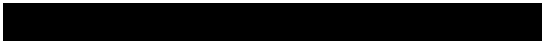 | | | | | | | | |
| 381 | 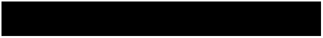 | | | | | | | | |
| 385 | 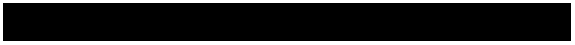 | | | | | | | | |
| 395 | 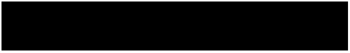 | | | | | | | | |
| 397 | 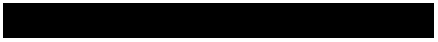 | | | | | | | | |
| 411 | 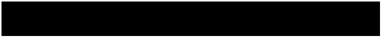 | | | | | | | | |
| 413 | 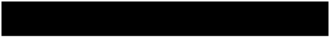 | | | | | | | | |
| 416 | 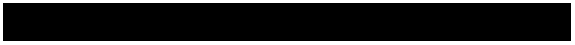 | | | | | | | | |
| 428 | 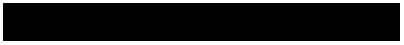 | | | | | | | | |
| 437 | 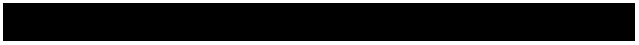 | | | | | | | | |
| 438 | 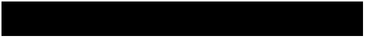 | | | | | | | | |
| 440 | 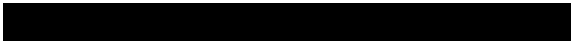 | | | | | | | | |
| 9 | 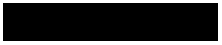 | | | | | | | | |
| 443 | 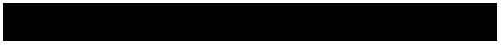 | | | | | | | | |
| 445 | 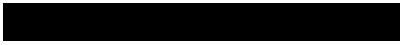 | | | | | | | | |
| | | 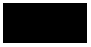 | | 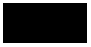 | | 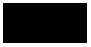 | | 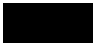 | |
| | | (−) | (+) | (−) | (+) | (−) | (+) | (−) | (+) |
| IDI | Description | PL-7B_BDE | PL-19B_BDE | PL-1B_BDE | PL-13B_BDE | PL-9B_BDE | PL-21B_BDE | PL-10B_BDE | PL-22B_BDE |
| 225 | AA116636_RIKEN_cDNA_1110013A05_gene | −1.3 | 1.2 | 1.6 | 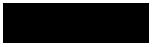 | −1.2 | 1.1 | −1 | 1.2 |
| 284 | AA237793_rat_regenerating_islet- | −25 | −18 | −16 | −16 | −28 | −10 | −27 | −24 |
| | derived,_mouse_homolog_1 | | | | | | | | |
| 28 | AA242201_serine_protease_inhibitor,_Kazal_type_3 | −5.3 | −6.5 | −9.5* | −5.2 | −3.6 | 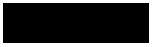 | 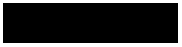 | 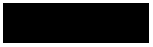 |
| 201 | AA867621_S-phase_kinase-associated_protein_2_(p45) | −1.7 | 1.3 | −1.2 | 1.5 | −1.9 | −1 | −2 | −1.4 |
| 232 | AI893740_glycine_N-methyltransferase | −1.3 | 1.1 | −1.4 | −1.4 | 1 | 1.1 | 1 | −1 |
| 353 | AA684073_ESTs | 1.2 | −1 | 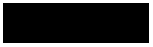 | −1 | 1.4 | 1.3 | 1.1 | −1.1 |
| 295 | AI551472_ESTs | −1.2 | 1.3 | −1.1+ | 1.5 | −1.2 | 1 | −1.1 | 1 |
| 113 | AA546645_cell_division_cycle_2_homolog_(s._pombe)- like_2 | −1.7 | 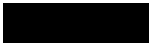 | 1.3 | 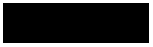 | −1.7 | 1.3 | −1.4 | 1 |
| 194 | 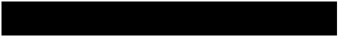 | −1.1 | −1.5 | −2.6 | −1.7 | −2.3 | −2.7 | −2.6 | −2.3 |
| 293 | AA497627_intersectin_(SH3_domain_protein_1A) | −1 | 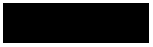 | 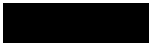 | 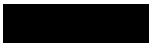 | −1.1 | 1.4 | −1.2 | 1 |
| 76 | 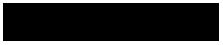 | 1.1 | 1.2+ | 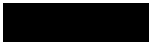 | 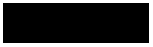 | 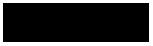 | 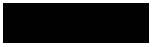 | 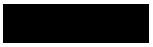 | 1.4 |
| 88 | AI122516_microtubule-associated_protein_6 | 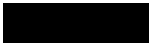 | 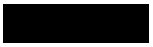 | 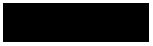 | 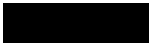 | 1.4 | 1.4 | −1.2 | [symbol] |
| 31 | AA638765_metallothionein_1 | −5.1 | −3.2 | 1.5 | 1.3 | −3 | −2.3 | −1 | −1.7 |
| 218 | W36474_metallothionein_2 | −1.8 | −1.9 | 1.4 | [symbol] | −2.9 | −2.5 | −1.7 | −2.7 |
| 268 | AA600634_signal_sequence_receptor,_delta | −2.1 | −1.7 | −2.4 | −2.3 | −1.3 | −1.5 | −1.2 | −1.3 |
| 48 | 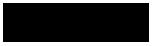 | 1.2 | 1.1 | 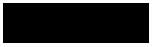 | 1.3 | 1.3 | 1.5 | 1.2 | −1 |
| 264 | AA790398_ribonuclease_I,_pancreatic | −44 | −23 | −15* | −27 | −45 | −11 | −39 | −36 |
| 9 | AA024217_Public_domain_EST | −3.9 | 1.1 | 1.4 | 1.5 | −2.5 | −1.3 | −1.9 | [symbol] |
| 87 | 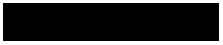 | 1.1 | 1.4 | 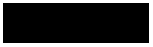 | 1.3 | 1.4* | 1.6 | 1.2 | 1.1 |
| 92 | 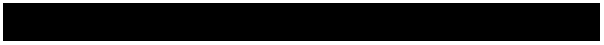 | −1.1 | 1.1 | 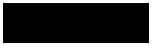 | 1.2 | 1 | 1.3 | −1.2 | −1.2 |
| 336 | 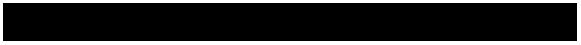 | 1.3 | −1.1 | 1.3 | −1 | 1.4 | 1.1 | 1 | −1.2 |
| 442 | 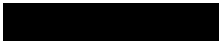 | 1.2+ | −1.1+ | 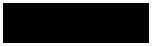 | 1 | 1.5 | 1.1+ | 1.4 | 1.2 |
| 356 | 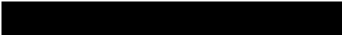 | 1.2 | 1 | [symbol] | −1.1 | [symbol] | 1 | 1.2 | 1 |
| 384 | 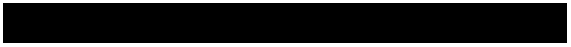 | −1 | −1.4 | 1.1 | −1.2 | 1.2 | −1.3 | 1.1 | −1.1 |
| 408 | 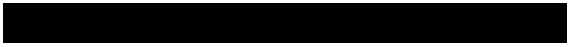 | 1.4 | −1.4 | 1.1 | −1.1 | 1.3 | −1.3 | 1.2 | −1.1 |
| 396 | 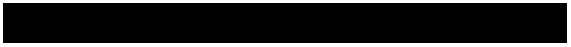 | 1.1 | −1.4 | 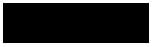 | 1.2 | 1.2 | −1.4 | −1 | −1.4 |
| 137 | AA067236_fibrlblast_growth_factor_receptor_1 | −1.1 | 1.1 | [symbol] | [symbol] | [symbol] | [symbol] | [symbol] | [symbol] |
| 119 | AA571365_Mus_musculus_MRPS6_mRNA_for_mito- | −2.5 | −1.2 | −1.4 | 1.4 | [symbol] | [symbol] | 1.5 | [symbol] |
| | chondrial_ribosomal_protein_S6,_partial_cds | | | | | | | | |
| 179 | AA013726_cathepsin_1 | −1.6 −2.5 | [symbol] | −2.5 −1.4 | [symbol] | −2.4 | −2.7 | | |
| 255 | 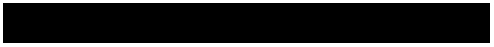 | −12 | −10 | −21 | −9.4 | −7.5 | −11 | −13 | −14 |
| 61 | AA717167_cytotoxic_T_lymphocyte- | −1.3 | −1.2 | −4.1 | −2.2 | −1.8 | −2.6 | −4.8 | −3.5 |
| | associated_protein_2_alpha | | | | | | | | |
| 63 | AA870247_mitogen_regulated_protein,_proliferin_3 | −1.7 | −3 | −4.6 | −2.6 | −1.7 | −1.8 | −2.6 | −2.6 |
| 190 | AI155437_Mus_musculus_20alpha- HSD_mRNA_for_20alpha- hydroxysteroid_dehydrogenase,_complete_cds | −1.3 | −1.4 | −1.3 | −1 | −1.5 | −1.4 | 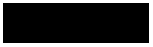 | 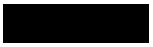 |
| 298 | AI597531_gap_junction_membrane_channel_protein_beta_2 | −1.8 | −2.3 | −1.9 | −2.4 | −1.8 | −2.7 | −1.3 | −1 |
| 81 | 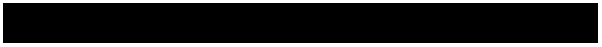 | −1.1 | −1.2 | −5 | −16 | −18 | −14 | −3.6 | −6.5 |
| 82 | 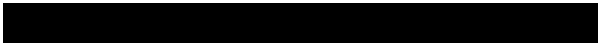 | −1.2 | −1.1 | −5 | −19 | −21 | −16 | −3.6 | −5.6 |
| 83 | 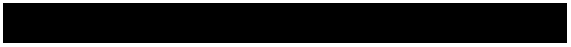 | −1.2 | −1 | −4.3 | −15 | −14 | −13 | −1.7 | −5.8 |
| 85 | 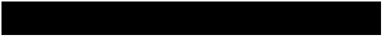 | [symbol] | −1.1 | −2.4 | −5.3 | −5.9 | −5.4 | −3 | −1.3 |
| 96 | 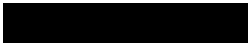 | 1.3 | 1.1 | 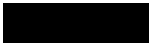 | 1.3 | −1 | −1.1 | −1.8 | −1.5 |
| 84 | W14953_kinesin-associated_protein_3 | −1.1 | 1.2 | 1.4 | −2.4 | −5.7 | −1.7 | −2.4 | −3 |
| 20 | AI324398_glycosylphosphatidylinositol_specific_phospholipase_D1 | −1.2 | 1 | −2.7 | −5.4 | −6.1 | −4.5 | −2.8 | −3.4 |
| 150 | AA166427_Mus_musculus_germline_immunoglobulin_gam- | −1.9 | −1.9 | −1.5 | −1.9 | −2.2 | −2.6 | −2.5 | −2.3 |
| | ma_constant_region_(IgG3)_mRNA | | | | | | | | |
| 208 | AA771355_ficolin_A | −2.1 | −1.9 | −2.5 | −2.4 | 1 | −1.2 | −3.6 | [symbol] |
| 97 | 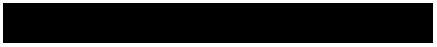 | −1+ | −1 | 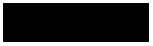 | 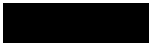 | 1.5 | 1.6 | −1.1 | −1 |
| 243 | AA145895_heat_shock_protein,_70_kDa_1 | 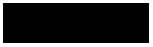 | −1.3 | −2.2 | −1.3 | 1.3 | 1.2 | −1.1 | −1.2 |
| 176 | AA239419_P_glycoprotein_2 | −1.9 | [symbol] | −2 | −2 | [symbol] | −2.9 | −2.7 | −2.6 |
| 363 | AA254235_complement_receptor_2 | 1.3 | −1.1 | [symbol] | 1.1 | 1.4 | 1.1 | 1.1 | 1 |
| 77 | AA612413_chiatinase_acidic | −9.4 | −11 | −42 | −18 | −11 | −28 | −58 | −17 |
| 114 | AI429054_ESTs | −1.5 | −1.1 | 1.1 | 1.1 | −1.4 | −1.1 | −1.5 | −1.5 |
| 158 | AA684403_ESTs | −1.3 | −1.3 | −1* | −1.5 | −1.1 | 1.2 | −2.1 | −1.7 |
| 191 | AA667301_troporin_I,_cardiac | −3.6 | −1.8 | −7.7 | −3.8 | −8.7 | −4 | −1.8** | [symbol] |
| 271 | AA210481_clusterin | −1.6 | −3.3 | 1.1 | −1.2 | −2.9 | −3.3 | −3.1 | −1.4 |
| 266 | AI120322_RIKEN_cDNA_1810055P16_gene | −1.9 | [symbol] | −2.2 | −2.6 | −1.2 | −1.3 | −1.2 | −1.2 |
| 267 | AA763351_death-associated_kinase_3 | −1.8 | [symbol] | [symbol] | [symbol] | −1.2 | −1.2 | −1.1 | −1.2 |
| 261 | 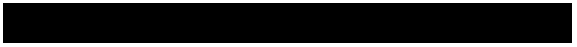 | −1.1 | 1 | −1.3 | −1.5 | 1.1 | 1.1 | 1 | 1.1 |
| 95 | AA119984_liver-specific_bHLH-Zip_transcription_factor | 1.2 | 1 | 1.1 | −1.9 | 1.2 | 1 | 1.1 | −1.3 |
| 144 | AI892747_glutathione_S-transferase,_alpha_1_(Ya) | −2.7 | −2.5 | −4.3 | −3.7 | −2 | −2.8 | 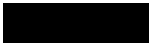 | 1 |
| 133 | AA606610_RIKEN_cDNA_2510002C21_gene | −1.2 | −1.2 | −3.8 | −3.6 | 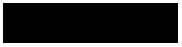 | 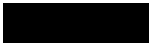 | −2 | [symbol] |
| 23 | AI385503_cysteine_rich_intestinal_protein | −1.1 | 1.1 | −3 | −2.3 | −1.5 | −1.5 | −2.9 | −2.8 |
| 159 | AA210237_Mus_musculus,_clone_MGC6377,_mRNA_com- | −1.4 | −1.8 | −1.4 | −1.8 | 1 | −1.2 | −2.2 | −3.1 |
| | plete_cds | | | | | | | | |
| 188 | W16059_glutathione_S-transferase_omega_1 | −1 | −1.2 | −2.2 | −2.5 | 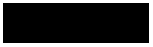 | 1.5 | −1.1 | −2.4 |
| 260 | W08923_cytochrome_c_oxidase_subunit_VI_a,_polypeptide_1 | −2.1 | −1.5 | [symbol] | [symbol] | −1.6 | −1.4 | 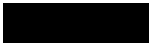 | 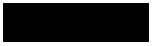 |
| 245 | AA002910_FBJ_osteosarcoma_oncogene | 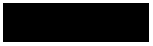 | 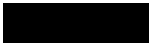 | 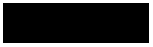 | 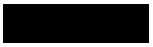 | 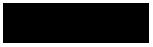 | 1.4 | −1.1 | 1.1 |
| 78 | W82228_annexin_A3 | 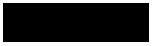 | 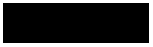 | −1.3 | −1.4 | 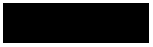 | 1.3 | −1.4 | −1.4 |
| 123 | AA437876_degenerative_spermatocyte_homolog_(Drosophila) | −1 | 1.1 | 1.1 | 1.2 | −1.4 | −1.3 | 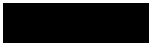 | 1.5 |
| 91 | 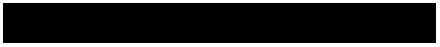 | | | | | | | | |
| 99 | 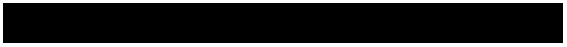 | | | | | | | | |
| 101 | 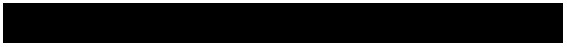 | | | | | | | | |
| 109 | AA727967_serine_(or_cysteine)_proteinase_in- | | | | | | | | |
| | hibitor,_clade_F_(alpha-2_amtiplasmin,_pigment_epithe- | | | | | | | | |
| | lium_derived_factor)_member_1 | | | | | | | | |
| 125 | AA725966_shingosine_kinase_I | | | | | | | | |
| 130 | AA062408_ESTs,_Moderately_similar_to_proteasome_in- | | | | | | | | |
| | hibitor_hP131_subunit_[H.sapiens] | | | | | | | | |
| 131 | AA619789_methylenetetrahydrofolate_dehydrogenase_(NAD+_de- | | | | | | | | |
| | pendent),_methenyltetrahydrofolate_cyclohydrolase | | | | | | | | |
| 138 | AA619953_ESTs,_Weakly_similar_to_T25027_hypo- | | | | | | | | |
| | thetical_protein_T20S3.5_-_Caenorhabditis_elegans_[C.elegans] | | | | | | | | |
| 140 | AI595630_RIKEN_cDNA_4930578I06_gene | | | | | | | | |
| 142 | AA106834_ESTs,_Weakly_similar_to_EP15_MOUSE_EPI- | | | | | | | | |
| | DERMAL_GROWTH_FACTOR_RECEPTOR_SUB- | | | | | | | | |
| | STRATE_SUBSTRATE_15_[M.musculus] | | | | | | | | |
| 145 | AA871395_Mus_musculus,_Simi- | | | | | | | | |
| | lar_to_ectonucleotide_pyrophosphatase/ | | | | | | | | |
| | phosphodiesterase_3._clone_IMAGE:3495326,_mRNA,_partial_cds | | | | | | | | |
| 162 | AA109528_ESTs,_Weakly_simi- | | | | | | | | |
| | lar_to_CAIC_MOUSE_COLLAGEN_AL- | | | | | | | | |
| | PHA_1(XII)_CHAIN_PRECURSOR_[M.musculus] | | | | | | | | |
| 169 | AI552688_ESTs,_Weakly_simi- | | | | | | | | |
| | lar_to_MIA_MOUSE_MELA- | | | | | | | | |
| | NOMA_DERIVED_GROWTH_REGULATORY_PRO- | | | | | | | | |
| | TEIN_PRECURSOR_[M.musculus] | | | | | | | | |
| 173 | AA823657_UDP-N-acetyl-alpha-D-galactosamine:(N- | | | | | | | | |
| | acetylneuraminyl)-galactosyl-N- | | | | | | | | |
| | acetylglucosaminylpolypeptide-beta-1,_4-N- | | | | | | | | |
| | acetylgalactosaminyltransferase | | | | | | | | |
| 210 | AA420359_DNA_segment,_Chr_6,_ERATO_Coi_109,_expressed | | | | | | | | |
| 236 | AA543982_interleukin_1_receptor,_type_1 | | | | | | | | |
| 238 | AI323261_lipase hepatic | | | | | | | | |
| 239 | AI425345_hydroxysteroid_17-beta_dehydrogenase_9 | | | | | | | | |
| 240 | AA274023_apoptosis_inhibitory_6 | | | | | | | | |
| 242 | AA067863_RIKEN_cDNA_2310057I18_gene | | | | | | | | |
| 246 | AA657278_Dnal_(Hsp40)_homolog,_subfamily_B,_member_1 | | | | | | | | |
| 276 | AA458425_Swi/SNF_related_matrix_associted,_actin_de- | | | | | | | | |
| | pendent_regulator_of_chromatin,_sub- | | | | | | | | |
| | family_a-like_1 | | | | | | | | |
| 279 | AA5179006_T-box_15 | | | | | | | | |
| 40 | AA717025_Mus_musculus_10_day_old_male_pan- | | | | | | | | |
| | creas_cDNA_RIKEN_full- | | | | | | | | |
| | length_enriched_library,_clone:1810009A17,_full_insert_se- | | | | | | | | |
| | quence | | | | | | | | |
| 43 | AA822106_ESTs,_Weakly_similar_to_CO8A_HUMAN_COM- | | | | | | | | |
| | PLEMENT_COMPLEMENT_COMPONENT_C8_AL- | | | | | | | | |
| | PHA_CHAIN_PRECURSOR_[H.sapiens] | | | | | | | | |
| 6 | AI447967_ESTs | | | | | | | | |
| 53 | AI614738_RIKEN_cDNA_2610301I15_gene | | | | | | | | |
| 55 | AI551391_ESTs,_Weakly_simi- | | | | | | | | |
| | lar_to_FVI_MOUSE_FRIEND_VIRUS_SUS- | | | | | | | | |
| | CEPTIBILITY_PROTEIN_1_[M.musculus] | | | | | | | | |
| 56 | AA473684_RIKEN_cDNA_C030002O17_gene | | | | | | | | |
| 57 | AI613926_twist_gene_homolog_(Drosophila) | | | | | | | | |
| 65 | AI549666_ESTs | | | | | | | | |
| 3 | AI553220_ESTs | | | | | | | | |
| 69 | AI549654_programmed_cell_death_10 | | | | | | | | |
| 73 | AA173562_ESTs,_Moderately_simi- | | | | | | | | |
| | lar_to_alias_DLC1_[H.sapiens] | | | | | | | | |
| 294 | AA655910_mast_cell_protease_2 | | | | | | | | |
| 297 | AI552048_ESTs | | | | | | | | |
| 299 | AA450831_BC1.2/adenovirus_E1B_19_kDa- | | | | | | | | |
| | interacting_protein_1,_NIP2 | | | | | | | | |
| 300 | AA517193_unc119_homolog_[C._elegans) | | | | | | | | |
| 307 | AA211954_RIKEN_cDNA_4930511A05_gene | | | | | | | | |
| 308 | AA530026_ESTs | | | | | | | | |
| 311 | AA438040_Public_domain_EST | | | | | | | | |
| 317 | AA470294_DNA_segment,_Chr_2,_ERATO_Doi_435,_ex- | | | | | | | | |
| | pressed | | | | | | | | |
| 320 | AA265406_nuclear_factor_I/B | | | | | | | | |
| 325 | AA210559_ESTs,_Weakly_similar_to_T34029_hypo- | | | | | | | | |
| | thetical_protein_C32F10.1_-_Caenorhabditis_elegans_[C.elegans] | | | | | | | | |
| 327 | AI553356_ESTs,_Moderately_similar_to_T00075_hypo- | | | | | | | | |
| | thetical_protein_KIAA0461_[H.sapiens] | | | | | | | | |
| 330 | AA254875_ESTs | | | | | | | | |
| 339 | AA596525_chromodomain_helicase_DNA_binding_protein_1 | | | | | | | | |
| 341 | 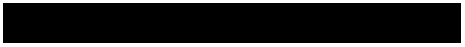 | | | | | | | | |
| 345 | 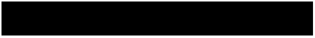 | | | | | | | | |
| 348 | 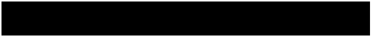 | | | | | | | | |
| 349 | AA438136_nuclear_localization_signal_protein_absent_in_velo- | | | | | | | | |
| | cardio-facial_patients | | | | | | | | |
| 352 | 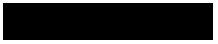 | | | | | | | | |
| 360 | 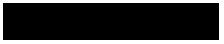 | | | | | | | | |
| 364 | 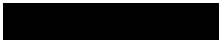 | | | | | | | | |
| 373 | 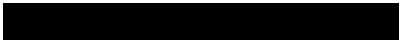 | | | | | | | | |
| 375 | 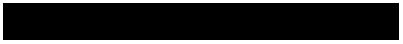 | | | | | | | | |
| 378 | 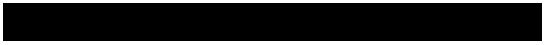 | | | | | | | | |
| 381 | 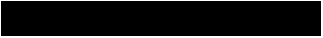 | | | | | | | | |
| 385 | 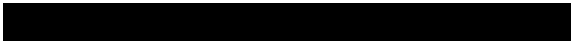 | | | | | | | | |
| 395 | 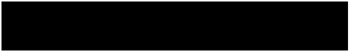 | | | | | | | | |
| 397 | 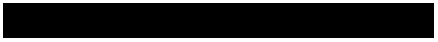 | | | | | | | | |
| 411 | 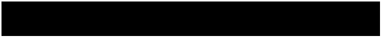 | | | | | | | | |
| 413 | 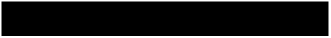 | | | | | | | | |
| 416 | 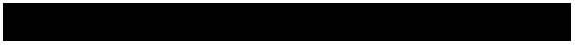 | | | | | | | | |
| 428 | 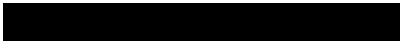 | | | | | | | | |
| 437 | 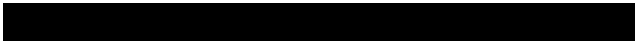 | | | | | | | | |
| 438 | 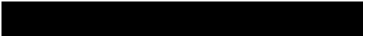 | | | | | | | | |
| 440 | 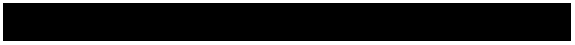 | | | | | | | | |
| 9 | 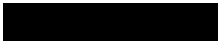 | | | | | | | | |
| 443 | 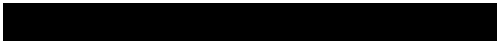 | | | | | | | | |
| 445 | 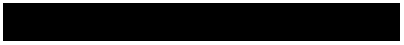 | | | | | | | | |

| | | 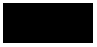 | | 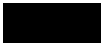 | | | | |
|---|---|---|---|---|---|---|---|---|
| | | (−) | (+) | (−) | (+) | | | |
| IDI | Description | PL-11B_BDE | PL-23B_BDE | PL-12B_BDE | PL-24B_BDE | Max Signal | Arrange Signal | Missed Values |
| 225 | AA116636_RIKEN_cDNA_1110013A05_gene | 1.1 | 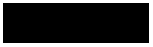 | 1.2* | 1 | [symbol] | [symbol] | 0 |
| 284 | AA237793_rat_regenerating_islet- | −24 | −20 | −29 | −23 | [symbol] | 3511.5 | 0 |
| | derived,_mouse_homolog_1 | | | | | | | |
| 28 | AA242201_serine_protease_inhibitor,_Kazal_type_3 | −2.5 | −2.5 | −2.9 | −7.1 | [symbol] | [symbol] | 0 |
| 201 | AA867621_S-phase_kinase-associated_protein_2_(p45) | −1.3 | 1.4 | −1.8 | −1.4 | 855 | 515.875 | 2 |
| 232 | AI893740_glycine_N-methyltransferase | −1.1 | −1.1 | −1 | −1.5 | [symbol] | [symbol] | 0 |
| 353 | AA684073_ESTs | 1.2 | 1 | 1.2 | −1.1 | [symbol] | [symbol] | 1 |
| 295 | AI551472_ESTs | −1.2 | 1.3 | −1.1 | −1 | 748 | [symbol] | 2 |
| 113 | AA546645_cell_division_cycle_2_homolog_(s._pombe)- like_2 | −1.4 | 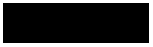 | −1.9 | −1.3 | [symbol] | [symbol] | 0 |
| 194 | 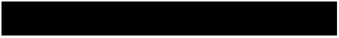 | −2.2 | −2.3 | −2.8 | −1.6* | [symbol] | [symbol] | 0 |
| 293 | AA497627_intersectin_(SH3_domain_protein_1A) | −1 | 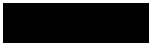 | −1.1 | 1.1 | [symbol] | [symbol] | 1 |
| 76 | 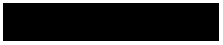 | 1.2 | 1.4* | 1.1 | 1.5 | 2482 | 312.417 | 12 |
| 88 | AI122516_microtubule-associated_protein_6 | 1.4 | 1.6 | 1.2* | 1.3 | 2589 | [symbol] | 1 |
| 31 | AA638765_metallothionein_1 | 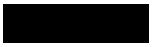 | 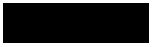 | −1.7 | −1.3 | 5922 | [symbol] | 0 |
| 218 | W36474_metallothionein_2 | 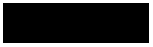 | 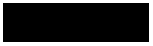 | −1.4 | −1.5 | 9727 | [symbol] | 0 |
| 268 | AA600634_signal_sequence_receptor,_delta | 1.6 | 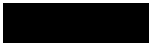 | 1.5 | 1.4 | [symbol] | [symbol] | 0 |
| 48 | 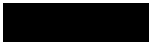 | 1.2 | 1.4 | 1.1 | 1.1 | 2212 | 713.833 | 1 |
| 264 | AA790398_ribonuclease_I,_pancreatic | −38 | −31 | −35 | −32 | [symbol] | [symbol] | 0 |
| 9 | AA024217_Public_domain_EST | −1.8 | 1.3 | −2.5 | −2.8 | 17710 | [symbol] | 0 |
| 87 | 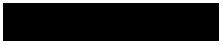 | −1* | 1.3 | 1.1* | 1.2 | 4170 | [symbol] | 1 |
| 92 | 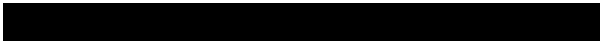 | 1.3 | 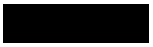 | −1 | −1.1 | [symbol] | [symbol] | 1 |
| 336 | 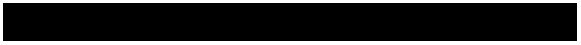 | 1.3 | −1 | 1.3 | −1 | [symbol] | 804 | 0 |
| 442 | 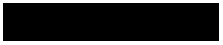 | 1.5 | −1 | 1.4* | −1 | 991 | [symbol] | 10 |
| 356 | 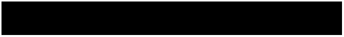 | 1.3 | 1 | 1.3* | −1 | [symbol] | [symbol] | 1 |
| 384 | 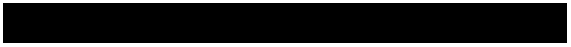 | 1.2 | −1.3 | 1.2 | −1.2 | [symbol] | [symbol] | 1 |
| 408 | 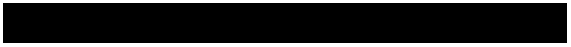 | 1.3 | −1.3 | 1.1 | −1.2 | 2214 | [symbol] | 0 |
| 396 | 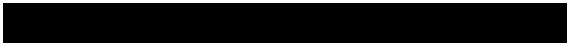 | 1.1 | −1.5 | 1.1 | −1.2 | 1540 | [symbol] | 0 |
| 137 | AA067236_fibrlblast_growth_factor_receptor_1 | 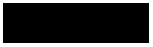 | 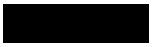 | 1.1 | 1.2 | [symbol] | [symbol] | 0 |
| 119 | AA571365_Mus_musculus_MRPS6_mRNA_for_mito- chondrial_ribosomal_protein_S6,_partial_cds | 1.2 | 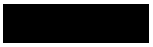 | −1.1 | 1.2 | 5119 | [symbol] | 0 |
| 179 | AA013726_cathepsin_1 | 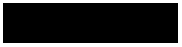 | 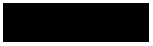 | −1.2 | −1.8 | [symbol] | [symbol] | 0 |
| 255 | 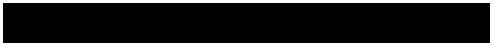 | 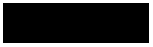 | 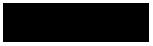 | −1.6 | −3.7 | [symbol] | [symbol] | 0 |
| 61 | AA717167_cytotoxic_T_lymphocyte- associated_protein_2_alpha | 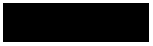 | 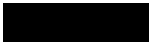 | −1.2 | −2.1 | 20879 | [symbol] | 0 |
| 63 | AA870247_mitogen_regulated_protein,_proliferin_3 | 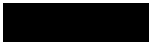 | 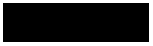 | −1.1 | −1.6 | [symbol] | [symbol] | 0 |
| 190 | AI155437_Mus_musculus_20alpha- HSD_mRNA_for_20alpha- hydroxysteroid_dehydrogenase,_complete_cds | 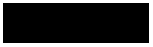 | 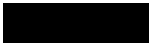 | 1.1 | −1.1 | 2107 | [symbol] | 1 |
| 298 | AI597531_gap_junction_membrane_channel_protein_beta_2 | 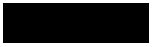 | 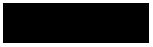 | 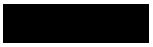 | 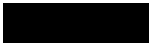 | [symbol] | [symbol] | 1 |
| 81 | 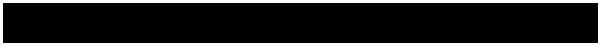 | −3 | −1.8 | −5.7 | −2.8 | [symbol] | [symbol] | 0 |
| 82 | 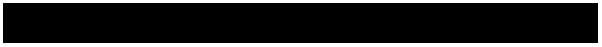 | −3 | −1.7 | −5.1 | [symbol] | [symbol] | [symbol] | 0 |
| 83 | 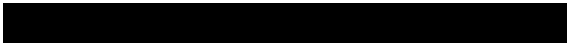 | −2.8 | −1.7 | −4.3 | −2.7 | [symbol] | [symbol] | 0 |
| 85 | 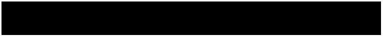 | −1.5 | −1.2 | −2.7 | −2.1 | [symbol] | [symbol] | 0 |
| 96 | 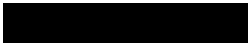 | −1.4 | −1.8 | −1.4 | −1.6 | [symbol] | [symbol] | 0 |
| 84 | W14953_kinesin-associated_protein_3 | −2.1 | −1.4 | −2.8 | −1.9 | 3571 | [symbol] | 0 |
| 20 | AI324398_glycosylphosphatidylinositol_specific_phospholipase_D1 | −2.8 | −1.8 | −3.4 | −2.2 | [symbol] | [symbol] | 0 |
| 150 | AA166427_Mus_musculus_germline_immunoglobulin_gam- | −2.1 | −2.3 | −1.3 | 1.1 | [symbol] | [symbol] | 1 |
| | ma_constant_region_(IgG3)_mRNA | | | | | | | |
| 208 | AA771355_ficolin_A | −1.9 | −1.8 | −1.4 | −1.2 | 5164 | 705.583 | 0 |
| 97 | 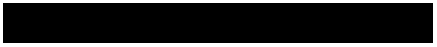 | −1 | −1.1 | 1.2* | 1.1 | [symbol] | [symbol] | 2 |
| 243 | AA145895_heat_shock_protein,_70_kDa_1 | −1.4 | −1.4 | −1.4 | −1.2 | [symbol] | [symbol] | 0 |
| 176 | AA239419_P_glycoprotein_2 | 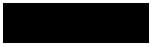 | 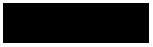 | −1.8 | −2.1 | [symbol] | [symbol] | 0 |
| 363 | AA254235_complement_receptor_2 | 1.2 | −1.2 | 1.2 | −1.1 | [symbol] | [symbol] | 1 |
| 77 | AA612413_chiatinase_acidic | −35 | −28 | −51 | −49 | [symbol] | [symbol] | 0 |
| 114 | AI429054_ESTs | −1.3 | 1.2 | −1.4 | 1 | [symbol] | [symbol] | 0 |
| 158 | AA684403_ESTs | −1.4 | −1.3 | −1.6 | −1.5 | [symbol] | [symbol] | 0 |
| 191 | AA667301_troporin_I,_cardiac | −6.5 | −6.4 | −3.2 | −4.4 | [symbol] | [symbol] | 0 |
| 271 | AA210481_clusterin | −5.3 | −4.4 | 1.4* | 1.2 | [symbol] | [symbol] | 0 |
| 266 | AI120322_RIKEN_cDNA_1810055P16_gene | 1.1 | 1.3 | 1.6 | 1.6 | [symbol] | [symbol] | 0 |
| 267 | AA763351_death-associated_kinase_3 | 1.1 | 1.4 | [symbol] | [symbol] | 11293 | [symbol] | 0 |
| 261 | 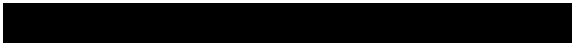 | 1.2 | 1.4 | 1.5 | 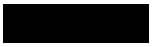 | [symbol] | [symbol] | 0 |
| 95 | AA119984_liver-specific_bHLH-Zip_transcription_factor | 1.4 | 1.3 | 1.4 | 1.5 | [symbol] | [symbol] | 0 |
| 144 | AI892747_glutathione_S-transferase,_alpha_1_(Ya) | −1.6 | −4.3 | −3.5 | −3.8 | [symbol] | [symbol] | 0 |
| 133 | AA606610_RIKEN_cDNA_2510002C21_gene | −1.7 | −1.7 | −3.4 | −1.2 | [symbol] | [symbol] | 0 |
| 23 | AI385503_cysteine_rich_intestinal_protein | −1.4 | −1.3 | −1.5 | [symbol] | [symbol] | [symbol] | 0 |
| 159 | AA210237_Mus_musculus,_clone_MGC6377,_mRNA_com- | −1.2 | −1.2 | −1.7 | −1.7 | 5894 | [symbol] | 0 |
| | plete_cds | | | | | | | |
| 188 | W16059_glutathione_S-transferase_omega_1 | −1.2 | −1.1 | −1.6 | −1.6 | [symbol] | [symbol] | 0 |
| 260 | W08923_cytochrome_c_oxidase_subunit_VI_a,_polypeptide_1 | 1 | 1.3 | 1.2 | 1.6* | 8377 | [symbol] | 0 |
| 245 | AA002910_FBJ_osteosarcoma_oncogene | 1.5 | 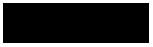 | 1 | 1.1 | 1782 | 725.417 | 0 |
| 78 | W82228_annexin_A3 | 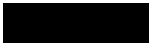 | 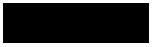 | 1 | −1 | 1488 | 381.667 | 1 |
| 123 | AA437876_degenerative_spermatocyte_homolog_(Drosophila) | 1.5 | 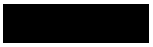 | −1.3 | −1.1 | 6711 | [symbol] | 0 |
| 91 | 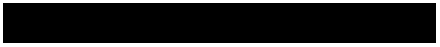 | | | | | | | |
| 99 | 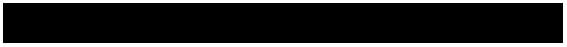 | | | | | | | |
| 101 | 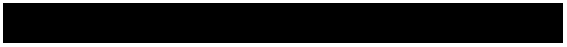 | | | | | | | |
| 109 | AA727967_serine_(or_cysteine)_proteinase_in- | | | | | | | |
| | hibitor,_clade_F_(alpha-2_amtiplasmin,_pigment_epithe- | | | | | | | |
| | lium_derived_factor)_member_1 | | | | | | | |
| 125 | AA725966_shingosine_kinase_I | | | | | | | |
| 130 | AA062408_ESTs,_Moderately_similar_to_proteasome_in- | | | | | | | |
| | hibitor_hP131_subunit_[H.sapiens] | | | | | | | |
| 131 | AA619789_methylenetetrahydrofolate_dehydrogenase_(NAD+_de- | | | | | | | |
| | pendent),_methenyltetrahydrofolate_cyclohydrolase | | | | | | | |
| 138 | AA619953_ESTs,_Weakly_similar_to_T25027_hypo- | | | | | | | |
| | thetical_protein_T20S3.5_-_Caenorhabditis_elegans_[C.elegans] | | | | | | | |
| 140 | AI595630_RIKEN_cDNA_4930578I06_gene | | | | | | | |
| 142 | AA106834_ESTs,_Weakly_similar_to_EP15_MOUSE_EPI- | | | | | | | |
| | DERMAL_GROWTH_FACTOR_RECEPTOR_SUB- | | | | | | | |
| | STRATE_SUBSTRATE_15_[M.musculus] | | | | | | | |
| 145 | AA871395_Mus_musculus,_Simi- | | | | | | | |
| | lar_to_ectonucleotide_pyrophosphatase/ | | | | | | | |
| | phosphodiesterase_3._clone_IMAGE:3495326,_mRNA,_partial_cds | | | | | | | |
| 162 | AA109528_ESTs,_Weakly_simi- | | | | | | | |
| | lar_to_CAIC_MOUSE_COLLAGEN_AL- | | | | | | | |
| | PHA_1(XII)_CHAIN_PRECURSOR_[M.musculus] | | | | | | | |
| 169 | AI552688_ESTs,_Weakly_simi- | | | | | | | |
| | lar_to_MIA_MOUSE_MELA- | | | | | | | |
| | NOMA_DERIVED_GROWTH_REGULATORY_PRO- | | | | | | | |
| | TEIN_PRECURSOR_[M.musculus] | | | | | | | |
| 173 | AA823657_UDP-N-acetyl-alpha-D-galactosamine:(N- | | | | | | | |
| | acetylneuraminyl)-galactosyl-N- | | | | | | | |
| | acetylglucosaminylpolypeptide-beta-1,_4-N- | | | | | | | |
| | acetylgalactosaminyltransferase | | | | | | | |
| 210 | AA420359_DNA_segment,_Chr_6,_ERATO_Coi_109,_expressed | | | | | | | |
| 236 | AA543982_interleukin_1_receptor,_type_1 | | | | | | | |
| 238 | AI323261_lipase hepatic | | | | | | | |
| 239 | AI425345_hydroxysteroid_17-beta_dehydrogenase_9 | | | | | | | |
| 240 | AA274023_apoptosis_inhibitory_6 | | | | | | | |
| 242 | AA067863_RIKEN_cDNA_2310057I18_gene | | | | | | | |
| 246 | AA657278_Dnal_(Hsp40)_homolog,_subfamily_B,_member_1 | | | | | | | |
| 276 | AA458425_Swi/SNF_related_matrix_associted,_actin_de- | | | | | | | |
| | pendent_regulator_of_chromatin,_sub- | | | | | | | |
| | family_a-like_1 | | | | | | | |
| 279 | AA5179006_T-box_15 | | | | | | | |
| 40 | AA717025_Mus_musculus_10_day_old_male_pan- | | | | | | | |
| | creas_cDNA_RIKEN_full- | | | | | | | |
| | length_enriched_library,_clone:1810009A17,_full_insert_se- | | | | | | | |
| | quence | | | | | | | |
| 43 | AA822106_ESTs,_Weakly_similar_to_CO8A_HUMAN_COM- | | | | | | | |
| | PLEMENT_COMPLEMENT_COMPONENT_C8_AL- | | | | | | | |
| | PHA_CHAIN_PRECURSOR_[H.sapiens] | | | | | | | |
| 6 | AI447967_ESTs | | | | | | | |
| 53 | AI614738_RIKEN_cDNA_2610301I15_gene | | | | | | | |
| 55 | AI551391_ESTs,_Weakly_simi- | | | | | | | |
| | lar_to_FVI_MOUSE_FRIEND_VIRUS_SUS- | | | | | | | |
| | CEPTIBILITY_PROTEIN_1_[M.musculus] | | | | | | | |
| 56 | AA473684_RIKEN_cDNA_C030002O17_gene | | | | | | | |
| 57 | AI613926_twist_gene_homolog_(Drosophila) | | | | | | | |
| 65 | AI549666_ESTs | | | | | | | |
| 3 | AI553220_ESTs | | | | | | | |
| 69 | AI549654_programmed_cell_death_10 | | | | | | | |
| 73 | AA173562_ESTs,_Moderately_simi- | | | | | | | |
| | lar_to_alias_DLC1_[H.sapiens] | | | | | | | |
| 294 | AA655910_mast_cell_protease_2 | | | | | | | |
| 297 | AI552048_ESTs | | | | | | | |
| 299 | AA450831_BC1.2/adenovirus_E1B_19_kDa- | | | | | | | |
| | interacting_protein_1,_NIP2 | | | | | | | |
| 300 | AA517193_unc119_homolog_[C._elegans) | | | | | | | |
| 307 | AA211954_RIKEN_cDNA_4930511A05_gene | | | | | | | |
| 308 | AA530026_ESTs | | | | | | | |
| 311 | AA438040_Public_domain_EST | | | | | | | |
| 317 | AA470294_DNA_segment,_Chr_2,_ERATO_Doi_435,_ex- | | | | | | | |
| | pressed | | | | | | | |
| 320 | AA265406_nuclear_factor_I/B | | | | | | | |
| 325 | AA210559_ESTs,_Weakly_similar_to_T34029_hypo- | | | | | | | |
| | thetical_protein_C32F10.1_-_Caenorhabditis_elegans_[C.elegans] | | | | | | | |
| 327 | AI553356_ESTs,_Moderately_similar_to_T00075_hypo- | | | | | | | |
| | thetical_protein_KIAA0461_[H.sapiens] | | | | | | | |
| 330 | AA254875_ESTs | | | | | | | |
| 339 | AA596525_chromodomain_helicase_DNA_binding_protein_1 | | | | | | | |
| 341 | 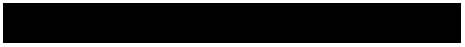 | | | | | | | |
| 345 | 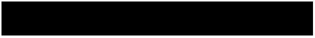 | | | | | | | |
| 348 | 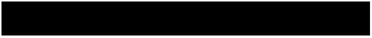 | | | | | | | |
| 349 | AA438136_nuclear_localization_signal_protein_absent_in_velo- | | | | | | | |
| | cardio-facial_patients | | | | | | | |
| 352 | 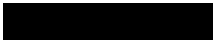 | | | | | | | |
| 360 | 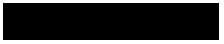 | | | | | | | |
| 364 | 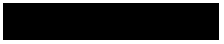 | | | | | | | |
| 373 | 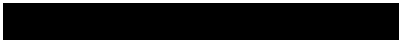 | | | | | | | |
| 375 | 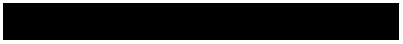 | | | | | | | |
| 378 | 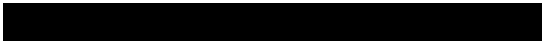 | | | | | | | |
| 381 | 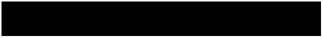 | | | | | | | |
| 385 | 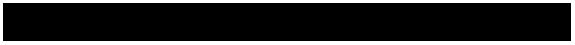 | | | | | | | |
| 395 | 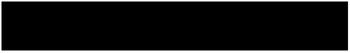 | | | | | | | |
| 397 | 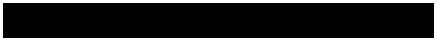 | | | | | | | |
| 411 | 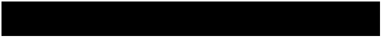 | | | | | | | |
| 413 | 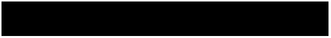 | | | | | | | |
| 416 | 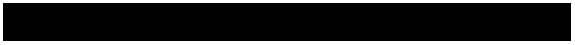 | | | | | | | |
| 428 | 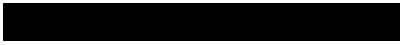 | | | | | | | |
| 437 | 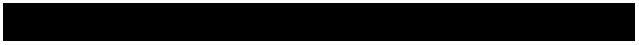 | | | | | | | |
| 438 | 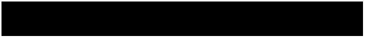 | | | | | | | |
| 440 | 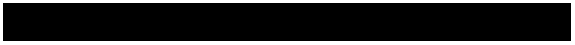 | | | | | | | |
| 9 | 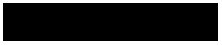 | | | | | | | |
| 443 | 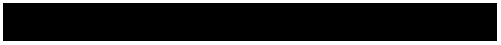 | | | | | | | |
| 445 | 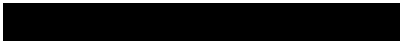 | | | | | | | |

| Description | ID | spl | panc | liver | stom | Intest | colon | brain | lung |
|---|---|---|---|---|---|---|---|---|---|
| AA242102 villin | MG225.GS.A- | | | | | | | | |
| | G.021AAQ0F8 | | | | | | | | |
| Al046562 lactate | MG225.GS.A- | | | | | | | | |
| dehydrogenase 3, | G,0217AAR9E3 | | | | | | | | |
| C chain, sperm | | | | | | | | | |
| specific | | | | | | | | | |
| Al326499 | MG225.GS.A- | | | | | | | | |
| epidermal growth | G.0211AAR3H3 | | | | | | | | |
| factor | | | | | | | | | |
| AA469630 choline kinase | MG225.GS.A- G.0210AAOJF5 | | | | 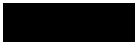 | | | | 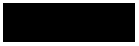 |
| W14224 N-myc | MG225.GS.A- | | | | | | | | |
| downstream | G.0210AAR8B11 | | | | | | | | |
| regulated 1 | | | | | | | | | |
| AA270885 parvalbumin | MG225.GS.A- G.0218AAR4C9 | | | | | | 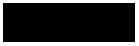 | | |
| AA760002 beta- | MG225.GS.A- | | | | | | | | |
| glucuronidase | G.021WAAP4G5 | | | | | | | | |
| structural, Beta- | | | | | | | | | |
| glucuronidase | | | | | | | | | |
| precursor | | | | | | | | | |
| W16059 | MG225.GS.A- | | | | | | | | |
| glutathione S- | G.0217AAR9C6 | | | | | | | | |
| transferase | | | | | | | | | |
| omega 1 | | | | | | | | | |
| W34620 kallikrein 26, Glandular kallitrein hK2 | MG225.GS.A- G.021BAAPUE10 | | | | | | | | 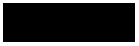 |
| AA466852 cysteine rich protein 61 | MG225.GS.A- G.021MAAPBA3 | | | | | | | | 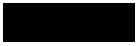 |
| AA445731 heat shock protein cognate 70, testis | MG225.GS.A- G.021NAAQ7H3 | | | | | | | | 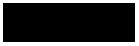 |
| AA002910 FBJ osteosarcoma oncogene, c-fos | MG225.GS.A- G.021NAAR1C4 | | | | | | | | 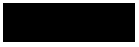 |
| AA289586 protein kinase C, beta | MG225.GS.A- G.0218AAR4C12 | | | | | | | 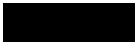 | 1.01996+ |
| AA119984 liver- specific bHLH- Zip transcription factor | MG225.GS.A- G.0218AAOFC12 | | | | | | | 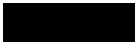 | 1.1 |
| AA051041 potassium voltage-gated channel, subfamily Q, member 2, Potassium voltage-gated channel subfamily KQT member 2, Neuroblastoma- specific potassium channel KQT-like 2, kcnq2 | MG225.GS.A- G.0218AAOFD10 | | | | | | | 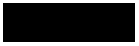 | 1.0726+ |
| AA222952 zuotin related factor 2, MPP11 | MG225.GS.A- G.0211AAR3D10 | | | | | | | 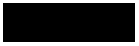 | 1.03133+ |
| AA231099 necdin | MG225.GS.A- G.0211AAR3E12 | | | | 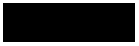 | | 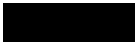 | 1.0 | −2.0 |
| AA518917 slug, chicken homolog | MG225.GS.A- G.0121DAAQEA12 | | | | | | 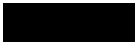 | −1.0 | −1.8 |
| Al893944 forkhead box G1, FOXG1B, c- Qin | MG225.GS.A- G.021BAAPUC12 | | | | | | 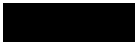 | 1.1 | −1.7 |
| AA982227 glutathione S- transferase, alpha 3 | MG225.GS.A- G.021OAAP8B7 | | | | | | 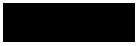 | −2.2 | 1.2 |
| AA619407 pancreatitis- associated protein | MG225.GS.A- G.021QAAQLA1 | | 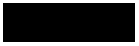 | | | 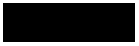 | −2.4 | −1.1 | −1.3 |
| AA871838 phospholipase A2, group IIA (platelets, synovial fluid) | MG225.GS.A- G.021QAAQLC11 | | | | | 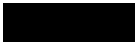 | 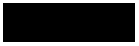 | −1.9 | −1.3 |
| W15890 guanylate cyclase activator 2 (guanylin 2, intestinal, healstable) | MG225.GS.A- G.0217AAR9B9 | | | | | 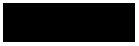 | −2.1 | −1.5 | −1.1 |
| AA793433 glutamyl aminopeptidase | MG225.GS.A- G.021LAAPGH1 | | | | | 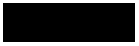 | −1.0 | 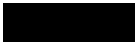 | 1.1 |
| AA239419 P glycoprotein 2 (mdr) | MG225.GS.A- G.021PAAPWF1 | | | | | 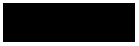 | 1.0 | 1.0 | 1.0 |
| AA265406 nuclear factor I/B | MG225.GS.A- G.021OAAQ2C3 | | | | 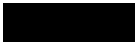 | 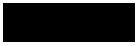 | −1.28917+˜ | 1.4 | 1.4 |
| AA683789 sirtuin 1 ((silent mating type information regulation 2, homolog) 1 (S. cerevisiae), hSIR2(SIRT1) | MG225.GS.A- G.021KAAPLB10 | | | | 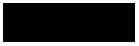 | 1.5 | −1.22848+˜ | 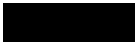 | 1.2 |
| AA756672 thyroid hormone responsive SPOT14 homolog (Rattus) | MG225.GS.A- G.021MAAR6F11 | | | 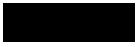 | −1.4 | −1.3 | −1.6 | −1.3 | −1.8 |
| Al425345 hydroxysteroid 17-beta dehydrogenase 9 | MG225.GS.A- G.021HAAQ1C2 | | | 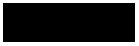 | 1.31173+ | 1.0 | 1.41016+˜ | 1.55264+ | 1.10611+ |
| AA027607 hydroxysteroid 17-beta dehydrogenase 2 | MG225.GS.A- G.021UAAR2A2 | | | 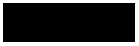 | 1.3 | 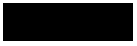 | 1.29025+˜ | 1.28348+ | 1.02353+ |
| AA210481 clusterin | MG225.GS.A- G.021TAAOlA1 | | 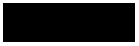 | 1.2 | −2.0 | −1.8 | −1.4 | 1.3 | 1.1 |
| AA790398 ribonuclease 1, pancreatic | MG225.GS.A- G.021PAAQQC11 | | 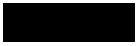 | −1.2 | −5.8 | −16.6 | −3.3 | 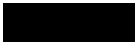 | −1.9 |

| Description | Blad | Kdny | pl-ut | Breast | Remarks |
|---|---|---|---|---|---|
| AA242102 villin | | | | 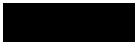 | a specific marker expressed in tumors of the digestive tract, renal proximal tubules, and hepatic bile duct, PMID: 11717541 |
| Al046562 lactate dehydrogenase 3, C chain, sperm specific | | | | 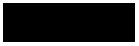 | Expressed in many cancers PMID: 12438276 |
| Al326499 epidermal growth factor | 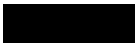 | | | 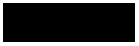 | EGF, fo review PMID: 12422312 |
| AA469630 choline kinase | 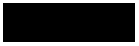 | | 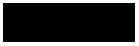 | 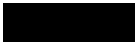 | Elevated in breast, lung, colorectal, and prostate tumors, PMID: 12176020 |
| W14224 N-myc downstream regulated 1 | | | | 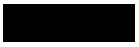 | Overexpressed in skin hyperplasia, PMID: 11746822, p53 resposive gene ?, androgene dependent gene |
| AA270885 parvalbumin | | | 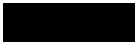 | −1.2 | Marker of specific tumors, (like chromophobe renal carcinoma, OPMID: 11504835) by expression on specific parental cell types, Neuroendocrine marker (PMID: 7902679) |
| AA760002 beta- glucoronidase structural, Beta- glucoronidase precursor | | 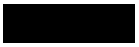 | −1.11695* | −1.1 | Elevation in poor differentiation colorectal tumor, PMID: 11717961, in pancreatic cancer PMID: 10961372, |
| W16059 glutathione S- transferase omega 1 | | 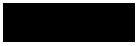 | −1.0 | 1.0 | unregulated in invasive human breast cancer lines, PMID: 12091914 |
| W34620 kallikrein 26, Glandular kallikrein hK2 | 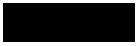 | 1.1 | −1.1 | −1.4 | Prostate cancer marker, PMID: 12142383, |
| AA466852 cysteine rich protein 61 | 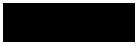 | −1.0 | 1.1 | 1.2 | overexpressed in invasive and metastatic human breast cancer cells and tissues, PMID: 12444554 |
| AA445731 heat | −1.0 | 1.1 | −1.2 | 1.1 | Negative results, No correlation with |
| shock protein | | | | | p53 expression and gastric cancer |
| cognate 70, | | | | | PMID: 10705241, Deletions or |
| testis | | | | | mutations in sporadic breast |
| | | | | | carcinoma, PMID: 10485459 |
| AA002910 FBJ osteosarcoma oncogene, c-fos | 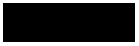 | 1.0 | −1.1 | −1.0 | Upregulated in many cancers |
| AA289586 protein kinase C, beta | −1.1 | −1.1 | 1.1 | 1.1 | Elevated in colon preneoplastic lesion PMID: 11245437, Decrease in preneoplastic prostate PMID: 9916928, Overexpressed by stromal dendritic cells in basal and squamous cell carcinoma.PMID: 9205496 |
| AA119984 liver- | 1.2 | 1.4 | 1.1 | 1.1 | Claimed as p53 target gene, PMID: |
| specific bHLH- | 11402317 | | | | |
| Zip transcription | | | | | |
| factor | | | | | |
| AA051041 | 1.10775+ | −1.1 | −1.21672+ | 1.20296+ | Found in neuroblastoma cells PMID: |
| potassium | | | | | 10455268 |
| voltage-gated | | | | | |
| channel, | | | | | |
| subfamily Q, | | | | | |
| member 2, | | | | | |
| Potassium | | | | | |
| voltage-gated | | | | | |
| channel subfamily | | | | | |
| KQT member 2, | | | | | |
| Neuroblastoma- | | | | | |
| specific | | | | | |
| potassium | | | | | |
| channel KQT-like | | | | | |
| 2, kcnq2 | | | | | |
| AA222952 zuotin | −1.14619+ | 1.0 | 1.0 | −1.0 | Amplified in Head and Neck |
| related factor 2, | | | | | Squamous Cell Cancer PMID: |
| MPP11 | | | | | 11034098 |
| AA231099 | −1.3 | −1.2 | −1.7 | 1.0 | Growth supressor |
| necdin | | | | | |
| AA51897 slug. | −1.4 | −1.2 | −1.8 | −1.3 | represses E-cadherin in breast |
| chicken | | | | | cancer PMID: 11912130 |
| homolog | | | | | |
| Al893944 | −1.5 | −1.1 | −1.6 | −1.1 | Protooncogene. analog of v-Qin |
| forkhead box | | | | | PMID: 11039897 |
| G1, FOXG1B, c- | | | | | |
| Qin | | | | | |
| AA982227 glutathione S- transferase, alpha 3 | 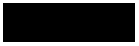 | 1.3 | −1.2 | −1.0 | Overexpressed in Warthin tumours and mucoepidermoid carcinomas of the human parotid gland, PMID: 8937651 |
| AA619407 | 1.0 | −1.2 | 1.3 | 1.1 | new marker for pancreatic ductal |
| pancreatitis- | | | | | adenocarcinoma, PMID: 11912167 |
| associated | | | | | |
| protein | | | | | |
| AA871838 phospholipase A2, group IIA (platelets, synovial fluid) | 1.5 | 1.2 | 1.3 | 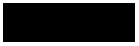 | elevated in prostatic intraepithelial neoplasia and adenocarcinoma, PMID: 11839587 |
| W15890 | −1.2 | −1.0 | 1.1 | 1.4 | Expression downregulated in |
| guanylate | | | | | intestine and colorectal |
| cyclase | | | | | adenocarcinoma, PMID: 9461126 |
| activator 2 | | | | | |
| (guanylin 2, | | | | | |
| intestinal, | | | | | |
| heatstable) | | | | | |
| AA793433 | 1.5 | 1.0 | 1.2 | −1.1 | Upregulated in cervial neoplasia, |
| glutamyl | | | | | PMID: 10838501 |
| aminopeptidase | | | | | |
| AA239419 P | 1.1 | −1.0 | −1.2 | 1.3 | Elevated in many cancers |
| glycoprotein 2 | | | | | |
| (mdr) | | | | | |
| AA265406 nuclear factor I/B | 1.2 | 1.3 | 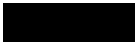 | 1.2 | Overexpressed in breast cancer PMID: 12208750, |
| AA683789 sirtuin | 1.4 | 1.1 | 1.3 | 1.3 | NAD-dependent p53 deacetylase, |
| 1 ((silent mating | | | | | antagonised p53-PML acticity, PMID: |
| type information | | | | | 1167523 |
| regulation 2, | | | | | |
| homolog) 1 (S. | | | | | |
| cerevisiae), | | | | | |
| hSIR2(SIRT1) | | | | | |
| AA756672 | −1.5 | −1.3 | 1.1 | −1.2 | Amplified and overexpressed in |
| thyroid hormone | | | | | lipogenic breast cancers, PMID: |
| responsive | 9618526 | | | | |
| SPOT14 | | | | | |
| homolog | | | | | |
| (Rattus) | | | | | |
| Al425345 | −1.0531+ | 1.13851+ | −1.05399+ | −1.13526+ | Expression elevated in epithelial |
| hydroxysteroid | | | | | ovarian tumors PMID: 8729977 |
| 17-beta | | | | | |
| dehydrogenase | | | | | |
| 9 | | | | | |
| AA027607 | 1.3 | 1.3 | −1.2 | 1.2 | Inversed correlation with breast |
| hydroxysteroid | | | | | cancer progression PMID: 11731426 |
| 17-beta | | | | | (type II decrease and type I increase |
| dehydrogenase | high risk) | | | | |
| 2 | | | | | |
| AA210481 | 1.1 | 1.4 | −1.2 | 1.2 | clusterin is a marker of anaplastic |
| clusterin | | | | | large cell lymphoma PMID: |
| | | | | | 1249802, expressed in human |
| | | | | | pancreatic cancer PMID: 12370533, |
| | | | | | breast carcinoma PMID: 10934144 |
| AA790398 | −4.1 | −1.1 | −1.2 | −1.1 | Expression elevated in pancreatic |
| ribonuclease 1, | | | | | adenocarcinoma and preneoplastic |
| pancreatic | | | | | lesions PMID: 11002220 |

| Prostate | Fold | Fold | Relation to cancer |
|---|---|---|---|
| AA982842 melanoma-inhibotory-activity protein, cartilage derived retinoic acid sensitive protein (Cdtrap), (MIA/CD-RAP) | 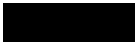 | | correlate with the progression of malignant melanoma and chondrosarcoma, PMID: 11918321 |
| AA107101 prostate stem cell antigen, PSCA | 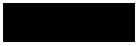 | | overexpressed in human prostate cancers, PMID: 12172427 |
| Al853055 Cystatin E (cystatin M) | | 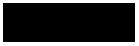 | Expressed in neoplastic epidermis PMID: 12100189 |
| M31885 Inhibitor of DNA binding 1, DNA- binding protein inhibitor ID-1 | | 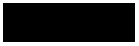 | Expression elevated in nasopharyngeal carcinoma cells. PMID: 12203366, Overexpressed in prostate cancer PMID: 11992094, overexpressed in medullary thyroid cancer. PMID: 1111462 |
| AW122874 PCNA | | 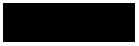 | Well known |
| alipoprotein D | 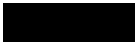 | 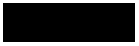 | Prostate tumor marker 9649289 |
| Secretory leukoprotease inhibitor gene | | 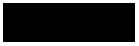 | Ovarian cancer candidate marker 11358798 |
| glutathione peroxidase 3 (plasma) | 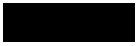 | 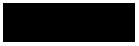 | Ovarian cancer candidate marker 11358798 |
| apolipoprotein E | 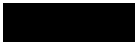 | 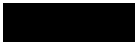 | Ovarian cancer candidate marker 11358798 |
| prostaglandin D2 synthase (21 kD, brain) | 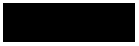 | 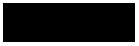 | Meningioma marker 11266526 |
| glutamyl aminopeptidase | 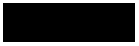 | 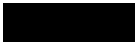 | Elevated in var. cervical tumors 10838501 |
| carbonic anhydrase VI | 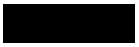 | 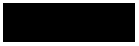 | Target for anti-cancer treatment 11310605 |

| | | | | 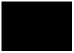 | 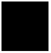 | 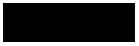 | | 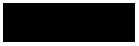 | |
|---|---|---|---|---|---|---|---|---|---|
| | | | | | | (−) | (+) | (−) | (+) |
| SEQ | | | | | | [symbol] | [symbol] | [symbol] | [symbol] |
| ID | | | corresponding human or mouse consensus | | | [symbol] | [symbol] | [symbol] | [symbol] |
| NO | ID: | mouse EST description | sequence (as in FIG. 5) | | | [symbol] | [symbol] | [symbol] | [symbol] |
| 1 | 161 | AA516927_sphinfosine_phosphate_lyase_1 | Homo sapiens sphinogosine-1-phosphate | intestine | blad | [symbol] | [symbol] | [symbol] | [symbol] |
| | | | lyase 1 (SGPL1), mRNA | | | | | | |
| 2 | 47 | AI552219_ESTs | Homo sapiens mRNA for putative cytoplasmatic protein (ORF1-FL21) as: gi-12214172/// [gi\|12214172\|emb\|AJ245876.1\|HSA245876] | stomach; bladder | blad | [symbol] | [symbol] | 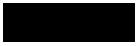 | [symbol] |
| 3 | 252 | 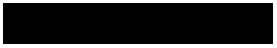 | Homo sapiens claudin 8 (CLDN8), mRNA cr: gi-21314655/// [Human_jongleur_201102.9749.CI] | mammary gland | blad, breast | [symbol] | [symbol] | 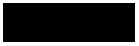 | [symbol] |
| 4 | 98 | 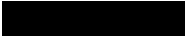 | Mus musculus LB9 gene, partial sequence cr: gi-687696/// [Mouse_jongleur_201102.10974.CI]; m_jongleur(identity): gi_1359159_gb_W57281.1_W57281 | brain | brain | [symbol] | [symbol] | [symbol] | [symbol] |
| 5 | 94 | 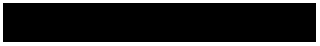 | Homo sapiens mRNA; cDNA DKFZp761J109 (from clone DKFZp761J109) cr: gi-21740184/// /cds=(0,429)/gb=AL834445/gi=21740184 /ug=Hs. 12600/len=3299 (Human_jongleur_201102.12676.CI] | brain | brain | [symbol] | [symbol] | [symbol] | [symbol] |
| 6 | 104 | 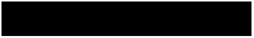 | Homo sapiens potassium voltage-gated channel, KQT-like subfamily, member 2 (KCNQ2), mRNA cr: gi-4758627/// [Human_jongleur_201102.8633.CI] | brain | brain | [symbol] | [symbol] | [symbol] | [symbol] |
| 7 | 122 | A1323053_forkhead_box_D3 | orth Homo sapiens forkhead box D3 | colon | brain | [symbol] | [symbol] | [symbol] | [symbol] |
| | | | (FOXD3), mRNA cr: gi-6912371/// | | | | | | |
| | | | [Human_jongleur_201102.379.C5]; | | | | | | |
| | | | h_jongleur(identity): | | | | | | |
| | | | gi_4057482_gb_AI323053.1_A1323053 | | | | | | |
| 8 | 115 | AA034678_rhotekin | Homo sapiens rhotekin (RTKN), mRNA | colon | brain | [symbol] | [symbol] | [symbol] | [symbol] |
| | | | cr: gi-19526470/// | | | | | | |
| | | | [Human_jongleur_201102.9437.CI] | | | | | | |
| 9 | 118 | AI323308_interleukin_3_receptor_alpha_chain | Mus musculus interleukin 3 receptor, | colon | brain | [symbol] | [symbol] | [symbol] | [symbol] |
| | | | alpha chain (II3ra), mRNA cr: gi-6680428/// | | | | | | |
| | | | [Mouse_jongleur_201102.3747.CI]; | | | | | | |
| | | | m_jongleur(identity): | | | | | | |
| | | | gi_4057737_gb_AI323308.1_AI323308 | | | | | | |
| 10 | 116 | AA518917_slug,_chicken_homolog | Homo sapiens snail 2 (SNAI2), mRNA | colon | brain | [symbol] | [symbol] | [symbol] | [symbol] |
| | | | cr: gi-24497625/// | | | | | | |
| | | | [Human_jongleur_201102.12116.CI] | | | | | | |
| 11 | 138 | AA546945_tyrosine_3- | Homo sapiens PDGFA associated protein I | colon | brain | [symbol] | [symbol] | [symbol] | [symbol] |
| | | monooxygenase/tryptophan_5- | (PDAPI), mRNA cr: gi-7657440/// | | | | | | |
| | | monooxygenase_activation_protein,_zeta_polypept | [Human_jongleur_201102.56.C1266] | | | | | | |
| | | ide | | | | | | | |
| 12 | 189 | AA45034_glutamine_synthetase_pseudogene_I | Homo sapiens glutamate-ammonia ligase | kidney | brain | [symbol] | [symbol] | [symbol] | [symbol] |
| | | | (glutamine synthase), clone MGC:20322 | | | | | | |
| | | | IMAGE:4137547, mRNA, complete cds | | | | | | |
| | | | cr: gi-15080156/// | | | | | | |
| | | | [Human_jongleur_201102.10208.C8] | | | | | | |
| 13 | 235 | AA415254_glutamate_oxaloacetate_transminase_{—} | Homo sapiens glutamine-oxaloacetic | liver | brain | [symbol] | [symbol] | [symbol] | [symbol] |
| | | I,_soluble | transaminase I, soluble (aspartate | | | | | | |
| | | | aminotransferase I) (GOTI), | | | | | | |
| | | | mRNA cr: gi-4504066/// | | | | | | |
| | | | [Human_jongleur_201102.747.CI] | | | | | | |
| 14 | 269 | AA437717_ESTs,_Moderately_similar_to_T34531 | Homo sapiens cDNA FLJ34227 fis, clone | pancreas | brain | [symbol] | [symbol] | [symbol] | [symbol] |
| | | _hypothetical_protein_DKFZp434P1215.1_[H.sapi | FCBBF3025098 si: gi-21749942/// | | | | | | |
| | | ens] | [Human_jongleur_201102.cl.6700.singlet] | | | | | | |
| 15 | 262 | AA575501_ESTs | vI94c08.rI Knowles Solter mouse | pancrease | brain | [symbol] | [symbol] | [symbol] | [symbol] |
| | | | blastocyst BI Mus musculus cDNA clone | | | | | | |
| | | | IMAGE:988334 5′: est(identity): | | | | | | |
| | | | gi_2350127_gb_AA575501.1_AA575501 | | | | | | |
| 16 | 263 | AA681081_ESTs | vuI3h03.xI Mus musculus cDNA, 3′ end | pancreas | brain | [symbol] | [symbol] | [symbol] | [symbol] |
| | | | as: gi-4602318/// clone=IMAGE:1180565 | | | | | | |
| | | | /clone_end=3′/gb=AI593270/gi=4602318 | | | | | | |
| | | | /ug=Mm.71891 /len=546 | | | | | | |
| | | | [gnl\|UG\|Mm#S746181]; | | | | | | |
| | | | m_jongleur(identity):gi_2663221_gb_ | | | | | | |
| | | | AA681081.I_AA681081 | | | | | | |
| 17 | 50 | A1464603_RIKEN_cDNA_071000E13_gene | oth Homo sapiens cyclic AMP-regulated | stomach, | brain | [symbol] | [symbol] | [symbol] | [symbol] |
| | | | phosphoprotein mRNA, complete cds si: | brain | | | | | |
| | | | gi-6563225/// | | | | | | |
| | | | [Human_jongleur_201102.c1.5922.singlet]; | | | | | | |
| | | | h_jongleur(identity): | | | | | | |
| | | | gi_4318633_gb_AI464603.1_AI464603 | | | | | | |
| 18 | 58 | AA231099_necdin | Homo sapiens necdin homolog (mouse) (NDN), mRNA cr: gi-10800414/// [Human_jongleur_201102.11950.CI] | stomach; colon | brain | [symbol] | [symbol] | 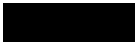 | [symbol] |
| 19 | 337 | AA000370_RIKEN_cDNA_4933438K12_gene | Homo sapiens hypothetical gene | stomach | brain | [symbol] | [symbol] | [symbol] | [symbol] |
| | | | BC008967 (BC008967), mRNA cr: | | | | | | |
| | | | gi-24308353/// | | | | | | |
| | | | [Human_jongleur_201102.7844.C2] | | | | | | |
| 20 | 333 | A1425572_ESTs_Highly_similar_to_zyginl_[R.nor | orth Homo sapiens fasiculation and | stomach | brain | [symbol] | [symbol] | [symbol] | [symbol] |
| | | vegicus] | elongation protein zeta I (zygin I) (FEZI), | | | | | | |
| | | | transcript variant | | | | | | |
| | | | I, mRNA cr: gi-17105402/// | | | | | | |
| | | | [Human_jongleur_201102.6883.C2]; | | | | | | |
| | | | h_jongleur(identity): | | | | | | |
| | | | gi_4271503_gb_A1425572.1 | | | | | | |
| | | | _A1425572 | | | | | | |
| 21 | 357 | AA517337_WASP_family_1 | Homo sapiens WAS protein family, member 1 (WASF1), mRNA cr: gi-4507912/// [Human_jongleur_201102.1128.C1] | stomach | brain | [symbol] | [symbol] | 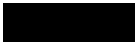 | [symbol] |
| 22 | 309 | AA498760_syntaxin_binding_protein_1 | ng45a05.s1 Homo sapiens cDNA 3′ end cr: gi-226375/// clone=IMAGE:937712/clone_end=3′/gb=AA524447 /gi=2265375 /ug=Hs.407835 /len=540[Human_jongleur_201102.592.C2] | stomach | brain | [symbol] | [symbol] | 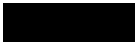 | [symbol] |
| 23 | 305 | AA588981_Mus_musculus_brain_cDNA,_clone_{—} | Homo sapiens, Similar to expressed | stomach | brain, | [symbol] | [symbol] | [symbol] | [symbol] |
| | | MNCb-1820 | sequence A1415388, clone | | | | | | |
| | | | IMAGE:5734009, mRNA, partial cds cr: | | | | | | |
| | | | gi-22477646/// /cds=(0,1754) | | | | | | |
| | | | /gb=BC036812 /gi=22477646 /ug= | | | | | | |
| | | | Hs.404915/len=3000 | | | | | | |
| | | | [Human_jongleur_201102.12931.C2] | | | | | | |
| 24 | 155 | AA656394_RIKEN_cDNA_1110025J15_gene | Homo sapiens cDNA FLJ10456 fis, clone | intestine | brain, | [symbol] | [symbol] | [symbol] | [symbol] |
| | | | NT2RP1001395 cr: gi-7022498/// | | | | | | |
| | | | [Human_jongleur_201102.9088.C2] | | | | | | |
| 25 | 292 | AA547242_undifferentiated_embryonic_cell_trans cription_factor_1 | Homo sapiens undifferentiated embryonic cell transcription factor 1 (UTF1), mRNA cr: gi-4507858/// [Human_jongleur_201102.841.C1] | stomach | brain, pl/ut | [symbol] | [symbol] | 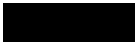 | [symbol] |
| 26 | 120 | AA656712_myosin,_hreavy_polypeptide_1,_skeleta | oth Homo sapiens myosin, heavy | colon | breast | [symbol] | [symbol] | [symbol] | [symbol] |
| | | l_muscle_adult | polypeptide 1, skeletal muscle, | | | | | | |
| | | | adult (MYH1), mRNA cr: gi-7669505/// | | | | | | |
| | | | [Human_jongleur_201102.1642.C5]; | | | | | | |
| | | | h_jongleur(strong | | | | | | |
| | | | homology):gi_2592866_gb_ | | | | | | |
| | | | AA656712.1_AA656712 | | | | | | |
| 27 | 202 | AA756672_thyroid_hormone_responsive_SPOT14 | Homo sapiens thyroid hormone responsive | liver | breast | [symbol] | [symbol] | [symbol] | [symbol] |
| | | _homolog_(Rattus) | (SPOT14 homolog, rat) (THRSP), mRNA | | | | | | |
| | | | cr: gi-4507496/// | | | | | | |
| | | | [Human_jongleur_201102.3891.C1] | | | | | | |
| 28 | 196 | A1789976_fatty_acid_Coenzyme_A_ligase,_long_c | oth Homo sapiens fatty-acid-Coenzyme A | liver | breast | [symbol] | [symbol] | [symbol] | [symbol] |
| | | hain_2 | ligase, long-chain 2 (FACL2), mRNA | | | | | | |
| | | | cr: gi-12669906/// | | | | | | |
| | | | [Human_jongleur_201102.2170.C2]; | | | | | | |
| | | | h_jongleur(strong homology): | | | | | | |
| | | | gi_5337692_gb_A1789976.1_ | | | | | | |
| | | | A1789976 | | | | | | |
| 29 | 253 | A1527496_Mus_musculus_mRNA,_compete_cds, | Mus musculus L-amino acid oxidase 1 | mammary | breast | [symbol] | [symbol] | [symbol] | [symbol] |
| | | _similar_to_L-amino_acid_oxidase | (Lao1), mRNA cr: gi-21941876/// | gland | | | | | |
| | | | [Mouse_jongleur_201102.7471.C1]; | | | | | | |
| | | | m_jongleur(identity): | | | | | | |
| | | | gi_4441631_gb_A1527496:1 | | | | | | |
| | | | _A1527496 | | | | | | |
| 30 | 250 | AA288467_glycosylation_dependent_cell_adhesio | Mus musculus 10 days lactation, adult | mammary | breast | [symbol] | [symbol] | [symbol] | [symbol] |
| | | n_molecule_1 | female mammary gland cDNA, RIKEN | gland | | | | | |
| | | | full-length enriched library, | | | | | | |
| | | | clone:D730050C14:glycosylation | | | | | | |
| | | | dependent cell adhesion molecule 1, | | | | | | |
| | | | full insert sequence cr: gi- | | | | | | |
| | | | 12862225 /ug=Mm.219621 /len=626 | | | | | | |
| | | | [Mouse_jongleur_201102.19220.C1]; | | | | | | |
| | | | m_jongleur(identity): | | | | | | |
| | | | gi_1937498_gb_AA288467.1_AA288467 | | | | | | |
| 31 | 152 | W56983_peripheral_myelin_protein,_22_kDa | Mus musculus peripheral myelin protein, | intestine | col, | [symbol] | [symbol] | [symbol] | [symbol] |
| | | | 22 kDa (Pmp22), mRNA cr: gi-6679394/// | | lung, | | | | |
| | | | [Mouse_jongleur_201102.1994.C1]; | | kidn | | | | |
| | | | m_jongleur(identity): | | | | | | |
| | | | gi_1358842_gb_W56983.1_{—W56983} | | | | | | |
| 32 | 181 | AA239727_ATP-binding_cassette_sub- | Mus musculus ATP-binding cassette, | intestine | col, | [symbol] | [symbol] | [symbol] | [symbol] |
| | | family_B_(MDR/TAP),_member_1 | sub-family B (MDR/TAP), member IB | | pl/ut | | | | |
| | | | (Abcb1b), mRNA cr: gi-6755045/// | | | | | | |
| | | | [Mouse_jongleur_201102.1138.C3]; | | | | | | |
| | | | m_jongleur(identity): | | | | | | |
| | | | gi_1863942_gb_AA239727.1_AA239727 | | | | | | |
| 33 | 107 | A1387457_retinol_binding_protein_2,_cellular | Mus musculus retinol binding protein 2, | colon | int | [symbol] | [symbol] | [symbol] | [symbol] |
| | | | cellular (Rbp2), mRNA cr: gi-6677686/// | | | | | | |
| | | | [Mouse_jongleur_201102.2997.C1]; | | | | | | |
| | | | m_jongleur(identity): | | | | | | |
| | | | gi_4198920_gb_A1385457.1 | | | | | | |
| | | | _A1385457 | | | | | | |
| 34 | 129 | A1550591_RIKEN_cDNA_2010003F10_gene | Mus musculus RIKEN cDNA 2010003F10 | colon | int | [symbol] | [symbol] | [symbol] | |
| | | | gene (2010003F10Rik), mRNA | | | | | | |
| | | | cr: gi-21312989/// | | | | | | |
| | | | [Mouse_jongleur_201102.5595.C1]; | | | | | | |
| | | | m_jongleur(identity): | | | | | | |
| | | | gi_4482954_gb_A1550591.1 | | | | | | |
| | | | _A1550591 | | | | | | |
| 35 | 160 | 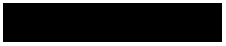 | Mus musculus similar to SI:PACKT90.1 (novel protein) ]Danio reno] (LOC223845), mRNA cr:gi-23621104/// [Mouse_jongleur_201102.21254.C1]; m_jongleur(identity): gi_2039712_gb_AA386758.1 _AA386758 | intestine | int | [symbol] | [symbol] | [symbol] | [symbol] |
| 36 | 17 | 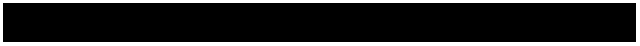 | Mus musculus fatty acid binding protein 2, intestinal (Fabp2), mRNA cr: gi-6679736/// [Mouse_jongleur_201102.16.C571]; m_jongleur(identity): gi_2272530_gb_AA529824.1 _AA529824 | colon; mammary gland | int, col | [symbol] | [symbol] | [symbol] | [symbol] |
| 37 | 19 | 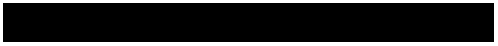 | Mus musculus defensin related cryptdin 6 (Defcr6), mRNA cr: gi-13937346/// [Mouse_jongleur_201102.3937.C10]; m_jongleur(identity): gi_2964618_gb_AA869173.1 _AA869173 | colon; placenta/ uterus | int, col | [symbol] | [symbol] | [symbol] | [symbol] |
| 38 | 136 | 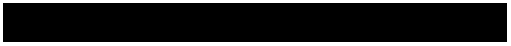 | Mus musculus defensin related cryptdin 6 (Defcr6), mRNA cr: gi-13937346/// [Mouse_jongleur_201102.3937.C10]; m_jongleur(identity): gi_2967359_gb_AA871914.1 _AA871914 | colon | int, col | [symbol] | [symbol] | [symbol] | [symbol] |
| 39 | 112 | 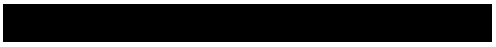 | Mus musculus defensin related cryptdin 6 (Defcr6), mRNA cr: gi-13937346/// [Mouse_jongleur_201102.3937.C10]; m_jongleur(identity): gi_2527048_gb_AA623172.1 _AA623172 | colon | int, col | [symbol] | [symbol] | [symbol] | [symbol] |
| 40 | 25 | 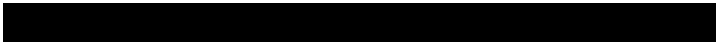 | Mus musculus defensin related cryptdin, related sequence 10 (Defcr-rs10), mRNA cr: gi-6681162/// [Mouse_jongleur_201102.3937.C1]; m_jongleur(identity): gi_2967086_gb_AA871461.1 _AA871641 | intestine, colon | int, col | [symbol] | [symbol] | [symbol] | [symbol] |
| 41 | 26 | 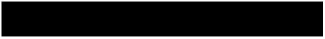 | Human RASF-A PLA2 gene encoding synovial phospholipase, exons 2 through 5 si: gi-190885/// [Human_jongleur_201102.c1.9676.singlet] | intestine; colon | int, col | [symbol] | [symbol] | [symbol] | [symbol] |
| 42 | 27 | 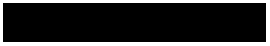 | Homo sapiens intelectin (ITLN), mRNA cr: gi-8923027/// [Human_jongleur_201102.4257.C2] | intestine; colon | int, col | [symbol] | [symbol] | [symbol] | [symbol] |
| 43 | 163 | 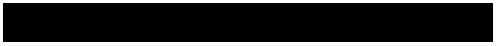 | Mus musculus defensin related cryptdin 5 (Defcr5), mRNA cr: gi-6681172/// [Mouse_jongleur_201102.3937.C3]; m_jongleur(identity): gi_2966855_gb_AA871410.1 _AA871410 | intestine | int, col | [symbol] | [symbol] | [symbol] | [symbol] |
| 44 | 177 | 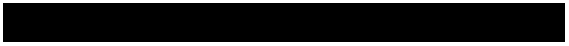 | orth Highly similar to NEP_HUMAN Neprilysin (Neutral endopeptidase) (NEP) (Enkephalinase) (Common acute lymphocyte leukemia antigen) (CALLA) (Neutral endopeptidase 24.11) (CD10) [H.sap cr:gi_8169687/// EST290530 Homo sapiens cDNA /gb=AW978421 /gi=8169687 /ug=Hs.307734 /len=675. [Human_jongleur_201102.11097.C4]; h_jongleur(identity): gi_2164395_gb_ | | | | | | |
| #AA450725. 1_AA450725 | intestine | int, col | [symbol] | [symbol] | [symbol] | [symbol] | | | |
| 45 | 167 | 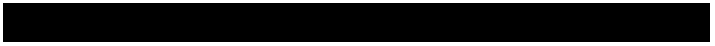 | Homo sapiens guanine nucleotide binding protein (G protein), alpha 11 (Gq class) (GNAII), mRNA cr: gi-4504036/// [Human_jongleur_201102.505.C2] | intestine | int, col | [symbol] | [symbol] | [symbol] | [symbol] |
| 46 | 178 | 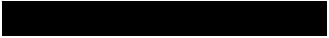 | Homo sapiens guanylin mRNA, complete cds cr:gi-183414/// /cds=(8,355) /gb=M97496 /gi=183414 /ug=Hs.778 /len=571 [Human_jongleur_201102.12385.C1] | intestine | int. col | [symbol] | [symbol] | [symbol] | [symbol] |
| 47 | 174 | 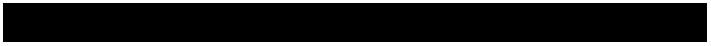 | Mus musculus defensin related to cryptdin, related sequence 10 (Defcr-rs10), mRNA cr: gi-6681162/// [Mouse_jongleur_201102.3937.C1]; m_jongleur(identity): gi_2967108_gb_AA871663.1 _AA871663 | intestine | int, col | [symbol] | [symbol] | [symbol] | [symbol] |
| 48 | 38 | 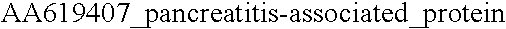 | Homo sapiens pancreatitis-associated protein (PAP), transcript variant 2, mRNA cr: gi-21070994/// [Human_jongleur_201102.6563.C1] | pancreas; intestine | int, col | [symbol] | [symbol] | 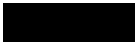 | [symbol] |
| 49 | 60 | 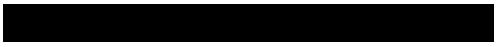 | Mus musculus defensin related cryptdin 6 (Defcr6), mRNA cr: gi-139377346/// [Mouse_jongleur_201102.3937.C10]; m_jongleur(identity): gi_2964626_gb_AA869181.1 _AA869181 | stomach; colon | int, col | [symbol] | [symbol] | [symbol] | [symbol] |
| 50 | 62 | 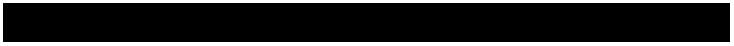 | Mus musculus 129 defensin-like gene 4C-4, complete cds cr: gi-531850/// [Mouse_jongleur_201102.3937.C5]; m_jongleur(identity): gi_2964611_gb_AA869166.1 _AA869166 | stomach; intestine | int, col | [symbol] | [symbol] | [symbol] | [symbol] |
| 51 | 106 | AA466153_ESTs | Homo sapiens cDNA FLJ33648 fis, clone | brain | int, kidn | [symbol] | [symbol] | [symbol] | [symbol] |
| | | | BRAMY2024497 si: gi-21749233/// | | | | | | |
| | | | [Human_jongleur | | | | | | |
| | | | _201102.c1.20905.singlet] | | | | | | |
| 52 | 170 | 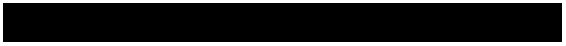 | Homo sapiens Sushi domain (SCR repeat) containing (BK65A6.2), mRNA cr: gi-22095356/// [Human_jongleur_201102.10520.C1] | intestine | kidn | [symbol] | [symbol] | [symbol] | [symbol] |
| 53 | 180 | 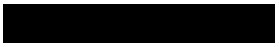 | Homo sapiens cDNA FLJ31240 fis, clone KIDNE2004981 si: gi-16550624/// [Human_jongleur_201102.c1.3017.singlet] | intestine | kidn | [symbol] | [symbol] | [symbol] | [symbol] |
| 54 | 29 | 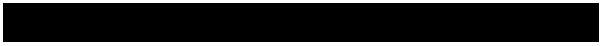 | Homo sapiens solute carrier family 22 (organic anion transporter), member 8 (SLC22A8), mRNA cr: gi-24497498/// [Human_jongleur_201102.8140.C2] | kidney; mammary gland | kidn | [symbol] | [symbol] | [symbol] | [symbol] |
| 55 | 187 | 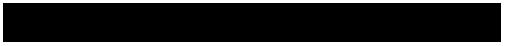 | Homo sapiens glucuronidase, beta (GUSB), mRNA cr: gi-4504222/// [Human_jongleur_201102.56.C168] | kidney | kidn | [symbol] | [symbol] | [symbol] | [symbol] |
| 56 | 219 | AA162217_pre-B-cell_colony-enhancing_factor | Homo sapiens pre-B-cell | liver | kidn | [symbol] | [symbol] | [symbol] | [symbol] |
| | | | colony-enhancing factor | | | | | | |
| | | | (PBEF), mRNA cr: gi-5031976/// | | | | | | |
| | | | [Human_jongleur_201102.6022.C1] | | | | | | |
| 57 | 222 | AA511089_glutathione_S-transferase_theta_2 | Homo sapiens glutathione S-transferase | liver | kidn | [symbol] | [symbol] | [symbol] | [symbol] |
| | | | theta 2 | | | | | | |
| | | | (GSTT2), mRNA cr: gi-7669495/// | | | | | | |
| | | | [Human_jongleur_201102.6032.C2] | | | | | | |
| 58 | 30 | 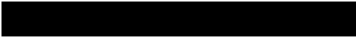 | Moderately similar to L1 repeat, Tf subfamily, member 18 [Mus musculus][M.musculus] as: gi-14793287/// 602959848F1 Mus musculus cDNA, 5′ end /clone=IMAGE:5125498 /clone_end=5′ /gb=B1247908 /gi=14793287/ ug=Mm.27508 /len =944. [gnl\|UG\|Mm#S2080812]; m_jongleur(identity): gi_2651629_gb_AA674392.1 _AA674392 | liver, colon | livr | [symbol] | [symbol] | | |
| # [symbol] | [symbol] | | | | | | | | |
| 59 | 213 | 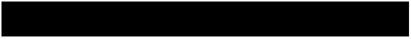 | mb87e07.yl Soares mouse p3MNF19.5 Mus musculus cDNA clone IMAGE:336420 5′; est(identity): gi_4216190_gb_A1390183.1 _A1390183 | liver | liv | [symbol] | [symbol] | [symbol] | [symbol] |
| 60 | 207 | 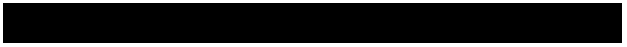 | Homo sapiens betaine-homocysteine methyltransferase (BHMT), mRNA cr: gi-4502406/// [Human_jongleur_201102.291.C1] | liver | liv | [symbol] | [symbol] | [symbol] | [symbol] |
| 61 | 205 | 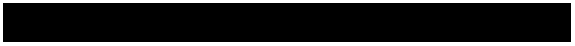 | orth Homo sapiens, Similar to uridine phosphorylase, clone MGC: 42328 IMAGE:4818300, mRNA, complete cds cr: gi-23272324/// cds=(216,1169) /gb=BC033529 /gi=23272324 /ug=Hs.128427 /len=2261 [Human_jongleur_201102.15166.C1]; h_jongleur(identity): gi_2345835_gb_AA572640.1 _AA572640 | liver | liv | [symbol] | [symbol] | [symbol] | [symbol] |
| 62 | 237 | 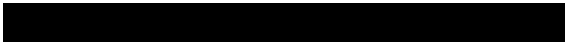 | Homo sapiens mRNA for MCM10 homolog, complete cds cr: gi-1152760/// [Human_jongleur_201102.7564C2] | liver | liv | [symbol] | [symbol] | [symbol] | [symbol] |
| 63 | 211 | 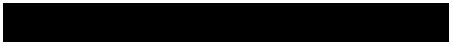 | Homo sapiens cytochrome P450, subfamily IIC (mephenytoin 4-hydroxylase), polypeptide 18 (CYP2C18), mRNA cr: gi-13699815/// [Human_jongleur_201102.11077.C7] | liver | liv | [symbol] | [symbol] | [symbol] | [symbol] |
| 64 | 233 | 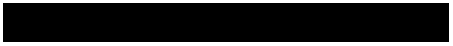 | Homo sapiens cytochrome P450, subfamily IIC (mephenytoin 4-hydroxylase), polypeptide 19 (CYP2C19), mRNA cr: gi-4503218/// [Human_jongleur_201102.1107.C2] | liver | liv | [symbol] | [symbol] | [symbol] | [symbol] |
| 65 | 230 | 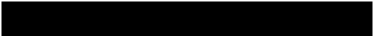 | Homo sapiens solute carrier family 27 (fatty acid transporter), member 5 (SLC27A5), mRNA cr: gi-13325056/// [Human_jongleur_201102.4565.C1] | liver liv | [symbol] | [symbol] | [symbol] | [symbol] | |
| 66 | 209 | W65070_ETSs | AV209686 Mus musculus cDNA, 3′ end cr: gi-6150394/// /clone=1700120N04 /clone_end=3′/gb=AV209686 /gi=6150394 /ug=Mm.140823 /len=167 [Mouse_jongleur_201102.18799.C1]; m_jongleur(identity): gi_1372720_gb_W65070.1_W65070 | liver | liv, col | [symbol] | 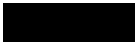 | 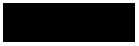 | [symbol] |
| 67 | 215 | W99034_ESTs | orth qc95h03.x1 Soares_preganat_uterus_NbHPU Homo sapiens cDNA clone IMAGE:1722005 3′; est(identity): gi_1428963_gb_W99034.1_W99034 | liver | liv, col | [symbol] | [symbol] | 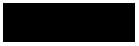 | [symbol] |
| 68 | 22 | AA066225_selenoprotein_P,_plasma,_1 | Homo sapiens selenoprotein P, plasma, 1 | intestine; | liv, col, | [symbol] | [symbol] | [symbol] | [symbol] |
| | | | (SEPP1), mRNA cr: gi-4885590/// | colon | kidn | | | | |
| | | | [Human_jongleur_201102.1857.C1] | | | | | | |
| 69 | 220 | W77249_selenium_binding_protei_1 | Homo sapiens selenium binding protein 1 | liver | liv, col, | [symbol] | [symbol] | [symbol] | [symbol] |
| | | | (SELENBP1), mRNA cr: gi-16306549/// | | lung, | | | | |
| | | | [Human_jongleur_201102.12047.C1] | | kidn | | | | |
| 70 | 186 | AA087441_RIKEN_cDNA_0610011L04_gene | Homo sapiens acetyl-Coenzyme A | kidney | liv, int | [symbol] | [symbol] | [symbol] | [symbol] |
| | | | acyltransferase 2 (mitochondrial | | | | | | |
| | | | 3-oxoacyl-Coenzyme A | | | | | | |
| | | | thiolase) (ACAA2), nuclear gene encoding | | | | | | |
| | | | mitochondrial protein, | | | | | | |
| | | | mRNA cr: gi-5174428/// | | | | | | |
| | | | [Human_jongleur_201102.11645.C1] | | | | | | |
| 71 | 21 | AA822098_apolipoprotein_A-1 | Homo sapiens apolipoprotein A-1 | intestine, | liv, int, | [symbol] | [symbol] | [symbol] | [symbol] |
| | | | (APOA1), mRNA cr: gi-4557320/// | colon | col | | | | |
| | | | [Human_jongleur_201102.263.C2] | | | | | | |
| 72 | 193 | AA096815_Public_domain_EST | orth Homo sapiens L-kynurenine/alpha- | kidney | liv, kidn | [symbol] | [symbol] | [symbol] | [symbol] |
| | | | aminoadipate aminotransferase (KATII), | | | | | | |
| | | | mRNA cr: gi-13518236/// | | | | | | |
| | | | [Human_jongleur_201102.6487.C1]; | | | | | | |
| | | | h_jongleur(strong homology): | | | | | | |
| | | | gi_1642517_gb_AA096815.1 | | | | | | |
| | | | _AA096815 | | | | | | |
| 73 | 33 | 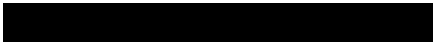 | Homo sapiens cytochrome P450, subfamily IIA (phenobarbital-inducible), polypeptide 13 (CYP2A13), mRNA cr: gi-19743563/// [Human_jongleur_201102.4706.C1] | liver, kidney | liv, kidn | [symbol] | [symbol] | [symbol] | [symbol] |
| 74 | 34 | 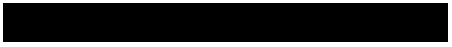 | Homo sapiens cytochrome P450, subfamily IIA (phenobarbital-inducible), polypeptide 13 (CYP2A13), mRNA cr: gi-19743563/// [Human_jongleur_201102.4706.C1] | liver, kidney | liv, kidn | [symbol] | [symbol] | [symbol] | [symbol] |
| 75 | 227 | A1892379_aminolevulinic_acid_synthase_1 | Moderately similar to HEMI_HUMAN 5- | liver | liv, lung | [symbol] | [symbol] | [symbol] | [symbol] |
| | | | aminolevulinic acid synthase, nonspecific, | | | | | | |
| | | | mitochondrial precursor (Delta- | | | | | | |
| | | | aminolevulinate synthase) (D cr: | | | | | | |
| | | | gi-5850819/// ws6h08.x1 Homo | | | | | | |
| | | | sapiens cDNA 3′ end | | | | | | |
| | | | /clone=IMAGE:2501727 | | | | | | |
| | | | /clone_end=3′ /gb=AW003903 | | | | | | |
| | | | /gi=5850819 /ug=Hs.407036 /len=678. | | | | | | |
| | | | [Human_jongleur_201102.9885.C2] | | | | | | |
| 76 | 199 | AA760070_Mus_musculus_triacylglycerol_hydrola | orth Homo sapiens carboxylase 3 (brain) | liver | liv, | [symbol] | [symbol] | [symbol] | [symbol] |
| | | se_mRNA,_complete_cds | (CES3), mRNA cr: gi-6912297/// | | lung, | | | | |
| | | | [Human_jongleur_201102.1674.C1]; | | kidn | | | | |
| | | | h_jongleur(identity): | | | | | | |
| | | | gi_2807864_gb_AA760070.1 | | | | | | |
| | | | _AA760070 | | | | | | |
| 77 | 212 | A1789916_hydroxysteroid_11- | Homo sapiens hydroxysteroid (11-beta) | liver | liv, | [symbol] | [symbol] | [symbol] | [symbol] |
| | | beta_dehydrogenase_1 | dehydrogenase 1 (HSD11B1), mRNA | | lung, | | | | |
| | | | cr: gi-5031764/// | | pl/ut | | | | |
| | | | [Human_jongleur_201102.8752.C1] | | | | | | |
| 78 | 80 | W11432_Leman_soiled-coil_protein | Homo sapiens angiomotin like 2 | bladder | lung | [symbol] | [symbol] | [symbol] | [symbol] |
| | | | (AMOTL2), mRNA cr: gi-7705577/// | | | | | | |
| | | | [Human_jongleur_201102.4649.C2] | | | | | | |
| 79 | 16 | 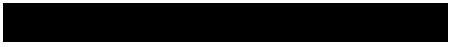 | Homo sapiens selenoprotein P, plasma, 1 (SEPP1), mRNA cr: gi-4885590/// [Human_jongleur_201102.1857.C1] | bladder, mammary gland | none | [symbol] | [symbol] | [symbol] | [symbol] |
| 80 | 79 | 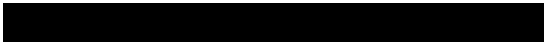 | Mus musculus demilune cell and parotid protein (Dcpp), mRNA cr: gi-9910187/// [Mouse_jongleur_201102.3917.C1]; m_jongleur(identity): gi_1318214_gb_W36921.1_W36921 | bladder | none | [symbol] | [symbol] | [symbol] | [symbol] |
| 81 | 74 | 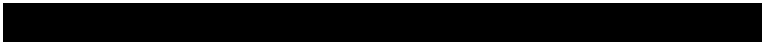 | Mus musculus submaxillary gland androgen regulated protein 1 (Smr1), mRNA cr: gi-14389424/// [Mouse_jongleur_201102.2306.C2]; m_jongleur(identity): gi_2803947_gb_AA756749.1 _AA756749 | bladder | none | [symbol] | [symbol] | [symbol] | [symbol] |
| 82 | 75 | 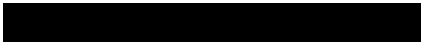 | Homo sapiens carbonic anhydrase VI gene, exon 6 si: gi-6652972/// [Human_jongleur_201102.c1.310.singlet] | bladder | none | [symbol] | [symbol] | [symbol] | [symbol] |
| 83 | 100 | 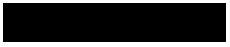 | mz99109.r1 Mus musculus cDNA, 5′ end as: gi-1904410/// /clone=IMAGE:721577 /clone_end=5′ /gb=AA267673 /gi=1904410 /ug=Mm.24323 /len=136 [gnl\|UG\|Mm#S161770]; m_jongleur(identity): gi_1904410_gb_AA267673.1 _AA267673 | brain | none | [symbol] | [symbol] | 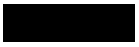 | [symbol] |
| 84 | 89 | 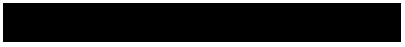 | Homo sapiens asparaginase-like 1 protein (ALP), mRNA cr: gi-23308566/// [Human_jongleur_201102.942.C2] | brain | none | [symbol] | [symbol] | [symbol] | [symbol] |
| 85 | 102 | 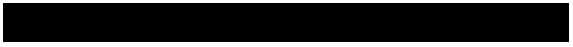 | Homo sapiens signal sequence receptor, gamma (translocon-associated protein gamma) (SSR3), mRNA cr: gi-6005883/// [Human_jongleur_201102.6056.C1] | brain | none | [symbol] | [symbol] | 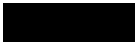 | [symbol] |
| 86 | 86 | 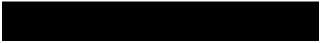 | Homo sapiens mRNA for K1AA0097 protein, partial cds cr: gi-603950/// /cds=(29,6145) /gb=D43948 /gi=603950 /ug=Hs.76989 /len=6628 [Human_jongleur_201102.686.C1] | brain | none | [symbol] | [symbol] | [symbol] | [symbol] |
| 87 | 103 | 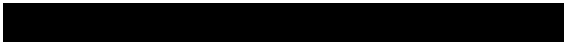 | Homo sapiens, Similar to K1AA1001 protein, clone MGC:8996 IMAGE:3882163, mRNA, complete cds as: gi-15214501/// [gi\|15214501\|gb\|BC012375.1\|BC012375] | brain | none | [symbol] | [symbol] | [symbol] | [symbol] |
| 88 | 93 | 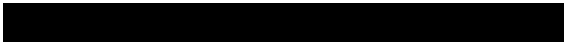 | Homo sapiens K1AA0102 gene product (K1AA0102), mRNA cr: gi-7661907/// [Human_jongleur_201102.6112.C1] | brain | none | [symbol] | [symbol] | 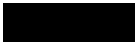 | [symbol] |
| 89 | 108 | 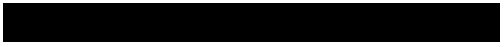 | orth Homo sapiens mesoderm specific transcript homolog (mouse) (MEST), mRNA; hum_rs_dna(identity): gi_4319104_gb_A1465074.1 _A1465074 | colon | none | [symbol] | [symbol] | [symbol] | [symbol] |
| 90 | 146 | 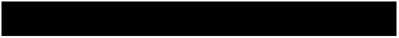 | Mus musculus 11 kDa secreted protein precursor, mRNA, complete cds cr: gi-8926323/// /cds=(23,631) /gb=AF272844 /gi=8926323 /ug=Mm.71887 /len=524 [Mouse_jongleur_201102.2852.C4]; m_jongleur(identity): gi_2745228_gb_AA727521.1 _AA727521 | colon | none | [symbol] | [symbol] | [symbol] | [symbol] |
| 91 | 192 | 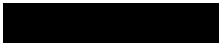 | NN513945 RIKEN full-length enriched, 10 days lactation, adult female mammary gland Mus musculus cDNA clone D730048K19; est(identity): gi_2812148_gb_AA762401.1 _AA762401 | kidney | none | [symbol] | [symbol] | [symbol] | [symbol] |
| 92 | 221 | 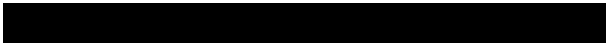 | Mus musculus retinoic acid early transcript 1, alpha (Raet1a), mRNA cr: gi-6679616/// [Mouse_jongleur_201102.2973.C1]; m_jongleur(identity): gi_1875322_gb_AA244388.1 _AA244388 | liver | none | [symbol] | 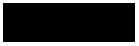 | [symbol] | [symbol] |
| 93 | 234 | 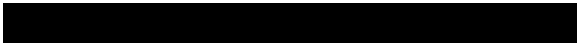 | Mus musculus peroxisomal biogenesis factor 11a (Pex11a), mRNA cr: gi-6755033/// [Mouse_jongleur_201102.1926.C1]; m_jongleur(identity): gi_1897427_gb_AA260931.1 _AA260931 | liver | none | [symbol] | [symbol] | [symbol] | [symbol] |
| 94 | 231 | 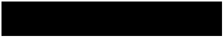 | Mus musculus ATP-binding cassette, sub-family A (ABC1), member 8b (Abca8b), mRNA cr: gi-23956075/// [Mouse_jongleur_201102.9242.C5]; m_jongleur(identity): gi_4407953_gb_A1509048.1 _A1509048 | liver | none | [symbol] | [symbol] | [symbol] | [symbol] |
| 95 | 224 | 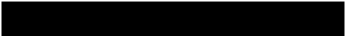 | Homo sapiens anillin, actin binding protein (scraps homolog, Drosphila) (ANLN), Mma | liver | none | [symbol] | [symbol] | [symbol] | [symbol] |
| 96 | 35 | 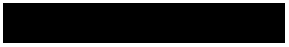 | zm73a01.s1 Homo sapiens cDNA 3′ end as: gi-1578801/// /clone=IMAGE:531240 /clone_end=3′/gb=AA071438 /gi=1578801 /ug=Hs.394289 /len=521. Weakly similar to preprokallikrein (AA- 24 to 238) [Homo sapiens] [H.sapiens][gnl\|UG\|HS#S481014] | lung, bladder | none | [symbol] | [symbol] | [symbol] | [symbol] |
| 97 | 259 | 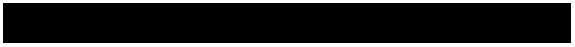 | Mus musculus 13 days embryo forelimb cDNA, RIKEN full-length enriched library, clone:5930412E23:unclassifiable transcript, full insert sequence cr: gi-12860486/// /cds=UNKNOWN /gb=AK020029 /gi=12860486 /ug=Mm.45194 /len=1349 [Mouse_jongleur_201102.13747.C1]; m_jongleur(identity): gi_2643256_gb_AA671177.1 _AA671177 | mammary gland | none | | | | |
| # [symbol] | [symbol] | [symbol] | [symbol] | | | | | | |
| 98 | 251 | 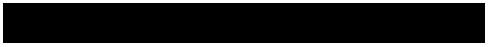 | Mus musculus cardiac responsive adriamycin protein (Crap), mRNA cr: gi-7304882/// [Mouse_jongleur_201102.4328.C1]; m_jongleur(identity): gi_2855454_gb_AA792499.1 _AA792499 | mammary gland | none | [symbol] | [symbol] | [symbol] | [symbol] |
| 99 | 1 | 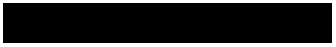 | Mus musculus choline kinase (Chk), mRNA cr: gi-7304958/// [Mouse_jongleur_201102.6600.C1]; m_jongleur(identity): gi_2196127_gb_AA469630.1 _AA469630 | pancreas; stomach; intestine; lung; bladder; placenta/ uterus; | none | [symbol] | [symbol] | 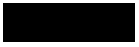 | [symbol] |
| 100 | 265 | 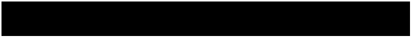 | orth Homo sapiens mutant guanine nucleotide-binding protein G(s). alpha subunit mRNA, complete cds si: gi-3142636/// [Human_jongleur_201102.c1.4319.singlet]; h_jongleur(identity): gi_3731437_gb_A1180799.1 _A1180799 | pancreas | none | [symbol] | [symbol] | [symbol] | [symbol] |
| 101 | 5 | 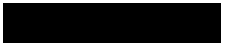 | AV304517 Mus musculus cDNA, 3′ end as: gi-16393611/// /clone=5730521N16 /clone_end=3′/gb=AV304517 /gi=16393611 /ug=Mm.73963 /len=653 [gnl\|UG\|Mm#S839272]; m_jongleur(identity): gi_2404877_gb_AA588976.1 _AA588976 | stomach; brain; placenta/ uterus; mammary gland | none | [symbol] | [symbol] | 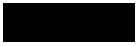 | [symbol] |
| 102 | 368 | 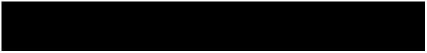 | Homo sapiens ATP-binding cassette, sub-family C (CFTR/MRP), member 10 (ABCC10), mRNA cr: gi-21541819/// [Human_jongleur_201102.5425.C1] | stomach | none | [symbol] | [symbol] | 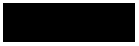 | [symbol] |
| 103 | 304 | 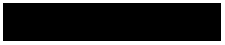 | Homo sapiens hypothetical protein GLJ10815 (FLJ10815), mRNA cr: gi-8922691/// [Human_jongleur_201102.6327.C1] | stomach | none | [symbol] | [symbol] | 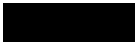 | [symbol] |
| 104 | 398 | 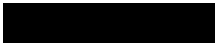 | vjl9d05.y1 Barstead mouse irradiated colon MPLRB7 Mus musculus cDNA clone IMAGE:922185 5′; est(identity):gi_4483340_gb _A15509771._A1550977 | stomach | none | [symbol] | [symbol] | 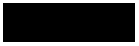 | [symbol] |
| 105 | 302 | A1509664_RIKEN_cDNA_2410174K12_gene | Homo sapiens mRNA for putative 4-6-3 protein si: gi-15216167/// [Human_jongleur_201102.c1.56.singlet] | stomach | none | [symbol] | [symbol] | 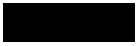 | [symbol] |
| 106 | 429 | 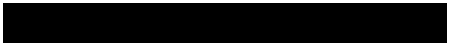 | Mus musculus zinc finger protein 101 (Zip101), mRNA cr: gi-6678628/// [Mouse_jongleur_201102.9115.C3]; m_jongleur(identity): gi_2456558_gb_AA607983.1 _AA607983 | stomach | none | [symbol] | [symbol] | 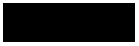 | [symbol] |
| 107 | 402 | 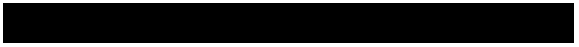 | orth Homo sapiens cDNA FLJ90669 fis, clone PLACE1005519, moderately similar to Homo sapiens STE20-like kinase 3 (mst-3) mRNA si: gi-22761052/// [Human_jongleur_201102.c1.3539.singlet]; h_jongleur(strong homology):gi_1671905_gb_AA1168291.1 _AA116829 | stomach | none | [symbol] | [symbol] | 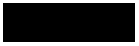 | [symbol] |
| 108 | 322 | 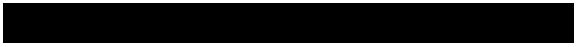 | Homo sapiens mRNA; cDNA DKFZp667B083 (from clone DKFZp667B083) cr: gi-19584391/// /gb=AL713686 /gi=19584391 /ug=Hs.184492 /len=3460 [Human_jongleur_201102.16601.C1] | stomach | none | [symbol] | [symbol] | [symbol] | [symbol] |
| 109 | 90 | 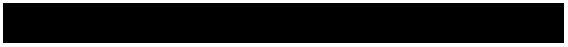 | Homo sapiens, clone MGC 33211 IMAGE:4829800, mRNA, complete cds cr: gi-23271237/// /cds=(319,1599) /gb=BC034999 /gi=23271237 /ug=Hs.88276 /len=1901 [Human_jongleur_201102.14048.C1] | brain | panc | [symbol] | [symbol] | 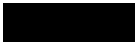 | 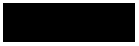 |
| 110 | 105 | 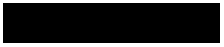 | Homo sapiens neurofilament, light polypeptide 68kDa (NEFL), mRNA cr: gi-5453761/// [Human_jongleur_201102.8785.C3] | brain | panc | [symbol] | [symbol] | 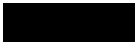 | 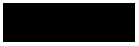 |
| 111 | 37 | 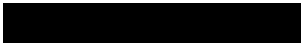 | orth zm73a01.s1 Homo sapiens cDNA 3′ end as: gi-1578801/// /clone=IMAGE:531240 /clone_end=3′ /gb=AA071438 /gi=1578801 /ug=Hs.394289 /len=521. Weakly similar to preprokallikrein (AA-24 to 238) [Homo sapiens][H.sapiens] (gnl\|UG\|Hs#S481014]; h_jongleur(identity): gi_2233597_gb_AA498574.1 _AA498574 | lung; bladder | panc | | | | |
| # [symbol] | [symbol] | 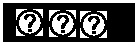 | 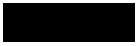 | | | | | | |
| 112 | 272 | 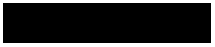 | Homo sapiens E3 ubiquitin ligase SMURF1 (SMURF1), mRNA cr: gi-20539202/// [Human_jongleur_201102.14736.C1] | pancreas | panc | [symbol] | [symbol] | 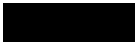 | 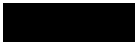 |
| 113 | 273 | 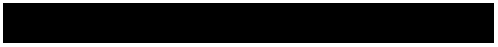 | orth Homo sapiens nescient helix loop helix 1 (NHLH1), mRNA cr: gi-19923328/// [Human_jongleur_201102.1103.C2]; h_jongleur(identity): gi_1843311_gb_AA223023.1 _AA223023 | pancreas | panc | [symbol] | [symbol] | 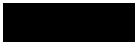 | 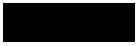 |
| 114 | 275 | 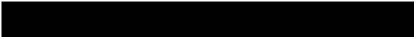 | Homo sapiens BCL2/adenovirus E1B 19kDa interacting protein 1 (BNP1P1), transcript variant BN1P1, mRNA cr: gi-4502440/// [Human_jongleur_201102.11130.C4] | pancreas | panc | [symbol] | [symbol] | 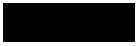 | 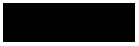 |
| 115 | 274 | 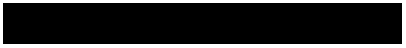 | Homo sapiens nucleophosmin (nuclear phosphoprotein B23, numatrin) (NPM1), mRNA cr: gi-20070168/// [Human_jongleur_201102.4957.CB3] | pancreas | panc | [symbol] | [symbol] | 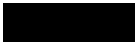 | 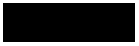 |
| 116 | 41 | 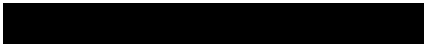 | orth Homo sapiens cDNA FLJ38286 fis, clone FCBBF3008153, highly similar to ALPHA-AMYLASE 2B PRECURSOR (EC 3.2.1.1) si: gi- 21754898/// [Human_jongleur_201102.c1.56.singlet]; h_jongleur(strong homology): gi_2891752_gb_AA821884.1 _AA821884 | spleen; brain | panc | [symbol] | [symbol] | 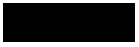 | 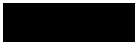 |
| 117 | 285 | AA982254_RIKEN_cDNA_2210010C04_gene | Moderately similar to TRY1_HUMAN Trypsin 1 precursor (Cationic trypsinogen) [H.sapiens] as: gi-2558387/// af07g07.s1 Homo sapiens cDNA 3′end /clone=IMAGE:1031004 /clone_end=3′/gb=AA635173 /gi=2558387 /ug=Hs.136626 /len=370. [gnl\|UG\|Hs#881001] | spleen | panc | [symbol] | [symbol] | 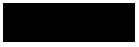 | 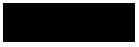 |
| 118 | 288 | AA674409_pancreatic_lipase_related_protein_1 | Homo sapiens pancreatic lipase-related protein 1 (PNL1PRP1), mRNA cr: gi-5453919/// [Human_jongleur_201102.6035.C1] | spleen | panc | [symbol] | [symbol] | 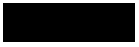 | 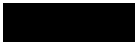 |
| 119 | 282 | AA675084_elastase_2 | Human pancreatic elastase IIA mRNA, complete cds | spleen | panc | [symbol] | [symbol] | 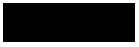 | 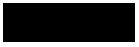 |
| 120 | 45 | AA155097_ESTs | Homo sapiens hypothetical protein FLJ10563 (FLJ10563), mRNA cr: gi-8922518/// [Human_jongleur_201102.3657.C1] | stomach; bladder | panc | [symbol] | [symbol] | 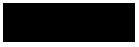 | 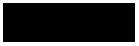 |
| 121 | 11 | AA153231_Public_domain_EST | orth qc96g08.x1 Homo sapiens cDNA 3′ end as: gi-3743449/// /clone=IMAGE:1722110 /clone_end=3′ /gb=A1192240 /gi=3743449 /ug=Hs.147175 /len=410 [gnl\|UG\|Hs#S1124018]; h_jongleur(identity): gi_1724900_gb_AA153231.1 _AA153231 | stomach; bladder; placenta/uteru s | panc | [symbol] | [symbol] | 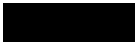 | 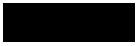 |
| 122 | 54 | 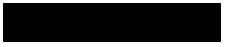 | orth Homo sapiens K1AA1576 protein (K1AA1576), mRNA cr: gi-2408256/// [Human_jongleur_201102.7803.C1]; h_jongleur(indentity): gi_2259229_gb_AA518686.1 _AA518686 | stomach; brain | panc | [symbol] | [symbol] | 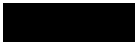 | 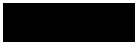 |
| 123 | 52 | 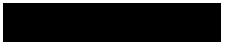 | orth Homo sapiens similar to intermediate filament-like protein MGC:2625, isoform 2; HOM-TES-103 tumor antigen-like; similar to CG15021 gene product (LOC126917), mRNA cr: gi-22041886/// [Human_jongleur_201102.4338.C3]; h_jongleur(identity): gi_4613754_gb_A1604587.1 _A1604587 | stomach; brain | panc | [symbol] | [symbol] | 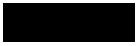 | 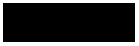 |
| 124 | 51 | 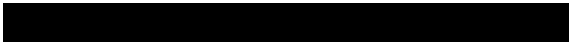 | orth Homo sapiens cullin 5 (CUL5), mRNA cr: gi-4503166/// [Human_jongleur_201102.833.C1]; h_jongleur(identity): gi_2071477_gb_AA412921.1 _AA412921 | stomach; brain | panc | [symbol] | [symbol] | 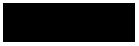 | 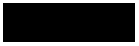 |
| 125 | 14 | AA073925_ESTs | orth AGENCOURT_7941852 NIH_MGC_67 Homo sapiens cDNA clone IMAGE:6172385 5′; est(identity): gi_{1'}1595672_gb_AA073925.1_AA073925 | stomach; intestine; lung | panc | [symbol] | [symbol] | 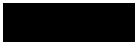 | 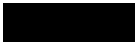 |
| 126 | 66 | AA412879_RIKEN_cDNA_C330006108_gene | Homo sapiens general transcription IIE, polypeptide 2 (beta subunit, 34kD) (GTF2E2), mRNA cr: gi-4504194/// [Human_jongleur_201102.511.C1] | stomach; kubg | panc | [symbol] | [symbol] | pget,0026 | 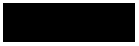 |
| 127 | 4 | A1481982_EST | vi20h04.x1 Mus musculus cDNA, 3′ end as: gi-4375208/// /clone=IMAGE:904375 /clone_end=3′/gb=A1481982 /gi=4375208 /ug=Mm.33142 /len=253 [gnl\|UG\|Mm#S442316]; m_jongleur(identity): gi_4375208_gb_A1481982.1 _A1481982 | stomach; lung; bladder; placenta/uteru s;mammary gland | panc | [symbol] | [symbol] | 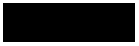 | 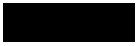 |
| 128 | 7 | AA623175_ESTs | Mus musculus SRY-box containing gene II (SoxII), mRNA cr: gi-6678064/// [Mouse_jongleur_201102.2327.C1]; m_jongleur(identity): gi_2527051_gb_AA623175.1 _AA623175 | stomach; lung; bladder; placenta/uteru s | panc | [symbol] | [symbol] | 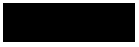 | 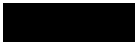 |
| 129 | 68 | AA210488_ESTs | mo86f04.x1 Mus musculus cDNA, 3′ end as: gi-4282215/// /clone=IMAGE:560479 /clone_end=3′/gb=A1448385 /gi=4282215 /ug=Mm.30681 /len=277 [gnl\|UG\|Mm#S431182]; m_jongleur(identity): gi_1807735_gb_AA210488.1 _AA210488 | stomach; mammary gland | panc | [symbol] | [symbol] | p 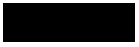 | 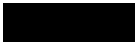 |
| 130 | 67 | AA617112_ESTs | vi22a05.r1 Mus musculus cDNA, 5′ end as: gi-2504317/// /clone=IMAGE:904496 /clone_end=5′/gb=AA617112 /gi=2504317 /ug=Mm.31089 /len=352 [gnl\|UG\|Mm#S246802]; m_jongleur(identity): gi_2504317_gb_AA617112.1 _AA617112 | mammary gland | panc | [symbol] | [symbol] | 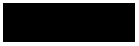 | 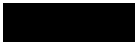 |
| 131 | 15 | AA623060_RIKEN_cDNA_24101241.17_gene | orth Homo sapiens hypothetical protein MGC17347 (MGC17347), mRNA cr: gi- 19923876/// [Human_jongleur_201102.4895.C1]; h_jongleur(identity): gi_2526936_gb_AA623060.1 _AA623060 | stomach; placenta/uteru s;mammary gland | panc | [symbol] | [symbol] | 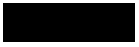 | 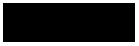 |
| 132 | 335 | A1604684_RIKEN_cDNA_4631422C13_gene | Mus musculus 0 day neonate skin cDNA, RIKEN full-length enriched library, clone:4631422C13:homolog to CDNA FLJ0352 FIS, CLONE NT2RM2001152, full insert sequence cr: gi-12852440/// /cds=(332,2401) /gb=AL014527 /gi=12852240 /ug=Mm.30857 /len=2692 [Mouse_jongleur_201102.12200.C1]; m_jongleur(identity): gi_4613851_ | | | | | | |
| #gb _A16046884 | stomach | panc | [symbol] | [symbol] | 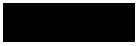 | 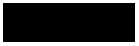 | | | |
| 133 | 354 | AA510659_ESTs | orth Homo sapiens cDNA: FLJ22823 fis, clone KA1A3974 cr: gi-10439347/// /gb=AK026476 /gi=1049347 /ug=Hs.398258 /len=1902 [Human_jongleur_201102.11450.C5]; h_jongleur(identity): gi_2248513_gb_AA510659.1 _AA510659 | stomach | panc | [symbol] | [symbol] | 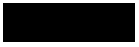 | 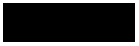 |
| 134 | 355 | AA023720_ESTs | mh78b12.r1 Mus musculus cDNA, 5′ end as: gi-1487635/// /clone=IMAGE:457055 /clone_end=5′/gb=AA023720 /gi=1487635 /ug=Mm.23708 /len=357 [gnl\|UG\|Mm#S56679]; m_jongleur(identity): gi_1487635_gb_AA023720.1 _AA023720 | stomach | panc | [symbol] | [symbol] | 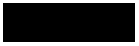 | 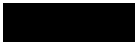 |
| 135 | 365 | AA450542_RIKEN_cDNA_8430430L24_gene | Mus musculus 16 days embryo lung cDNA, RIKEN full-length enriched library, clone: 8430430L24.homolog to HYPOTHETICAL 46.9 KDA PROTEIN, full insert sequence cr: gi-12858160/// /cds=(63,1325) /gb=AK018452 /gi=12858160 /ug=Mm.182246 /len=2151 [Mouse_jongleur_201102.17388.C1]; m_jongleur(identity): gi_2164122_gb | | | | | | |
| #_AA450452.1 _AA450452 | stomach | panc | [symbol] | [symbol] | 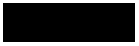 | 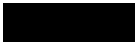 | | | |
| 136 | 372 | AA562544_RIKEN_cDNA_110035L05_gene | Mus musculus RIKEN cDNA 1110035L05 gene (1110035L05Rik), mRNA cr: gi-13385631/// [Mouse_jongleur_201102.1384.C1]; m_jongleur(identity): gi_2334009_gb_AA562544.1 _AA562544 | stomach | panc | [symbol] | [symbol] | 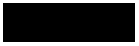 | 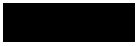 |
| 137 | 376 | A1325781_ESTs_Weakly_similar_to_S37771_ank tnin_erythrocyte_-_mouse_[M.musculus] | Homo sapiens cDNA FLJ20288 fis, clone HEP04414 cr: gi-7020281/// [Human_jongleur_201102.861.C3] | stomach | panc | [symbol] | [symbol] | 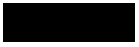 | 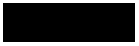 |
| 138 | 389 | AA066256_Public_domain_EST | Mus musculus 16 days embryo lung cDNA, RIKEN full-length enriched library, clone: 8430411F12:unclassifiable transcript, full insert sequence cr: gi-12858086/// /cds=UNKNOWN /gb=AK018402 /gi=12858086 /ug=Mm.196522 /len=1085 [Mouse_jongleur_201102.20299.C1]; m_jongleur(identity): gi_1563181_gb_AA066256.1 _AA066256 | | | | | | |
| #stomach | panc | [symbol] | [symbol] | 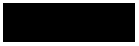 | 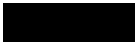 | | | | |
| 139 | 391 | AA267824_Public_domain_EST | Homo sapiens T-cell activation MFKB-like protein (TA-NFKBH), mRNA cr: gi-21245099/// [Human_jongleur_201102.2906.C1] | stomach | panc | [symbol] | [symbol] | 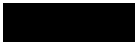 | 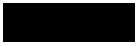 |
| 140 | 399 | AA833196_RIKEN_cDNA_4932431F02_gene | orth SP1090 Homo sapiens cDNA 3′ end as: gi-8888427/// /clone_end=3′ /gb=AW064490 /gi=8888427 /ug=Hs.330544 /len=325 [gnl\|UG\|Hs#S2133178]; h_jongeur(identity): gi_2906924_gb_AA833196.1 _AA833196 | stomach | panc | [symbol] | [symbol] | 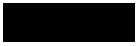 | 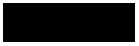 |
| 141 | 405 | A1608071_RIKEN_cDNA_2900017D14_gene | Homo sapiens protein phosphatase 2C alpha 2 mRNA, complete cds si: gi-3372871/// [Human_jongleur_201102.c1.5377.singlet] | stomach | panc | [symbol] | [symbol] | 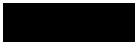 | 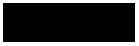 |
| 142 | 407 | AA395924_ESTs | none | stomach | panc | [symbol] | [symbol] | 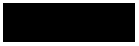 | 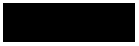 |
| 143 | 418 | AA270832_ESTs | va85g07.r1 Soares mouse NML Mus musculus cDNA clone IMAGE:738204 5′; est(identity): gi_1909339_gb_AA270832.1_AA270832 | stomach | panc | [symbol] | [symbol] | 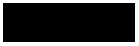 | 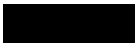 |
| 144 | 420 | A1463731_ESTs | orth Homo sapiens IDN3 protein (IDN3), transcript variant A, mRNA cr: gi-19718748/// [Human_jongleur_201102.11864.C1]; h_jongleur_201102.11864.C1]; h_jongleur(identity): gi_4317761_gb_A1463731.1 _A1463731 | stomach | panc | [symbol] | [symbol] | 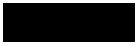 | 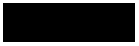 |
| 145 | 422 | AA273401_Public_domain_EST | zm97107.s1 Homo sapiens cDNA 3′ end cr: gi-1681430/// /clone=IMAGE:545893 /clone_end=3′/gb=AA079486 /gi=1618430 /ug=Hs. 394417 /len=481 [Human_jongleur_201102.20363.C1] | stomach | panc | [symbol] | [symbol] | 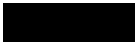 | 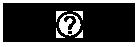 |
| 146 | 374 | A1549656_RIKEN_cDNA_3632413F13_gene | orth Homo sapiens U4/U6-associated RNA splicing factor (HPRP3P), mRNA cr: gi-4758555/// [Human_jongleur_201102.961.C1]; h_jongleur(identity): gi_4482019_gb_A1549656.1 _A1549656 | stomach | panc | [symbol] | [symbol] | 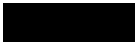 | 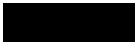 |
| 147 | 303 | AA688667_ESTs_Weakly_similar_to_Inv_[M.mu sculus] | Homo sapiens, clone IMAGE:5265597, mRNA si:gi-23272510/// [Human_jongleur _201102.c1.18267.singlet] | stomach | panc | [symbol] | [symbol] | 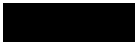 | 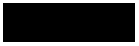 |
| 148 | 388 | AA269742_RIKEN_cDNA_1110065A22_gene | Homo sapiens similar to Gliacolin (LOC222404), mRNA as: gi-20473752/// [gi\|20473752\|ref\|XM_169005.1\|] | stomach | panc | [symbol] | [symbol] | 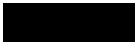 | 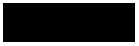 |
| 149 | 403 | AA220774_ESTs | orth nf70601.x5 Homo sapiens cDNA 3′end as:gi-5053451/// /clone=IMAGE:925225 /clone_end=3′ /gb=A1732338 /gi=503451 /ug=Hs.105821 /len=558 [gnl\|UG\|Hs#S1425572]; h_jongleur(strong homology): gi_1838594_gb_AA220774.1 _AA220774 | stomach | gene | [symbol] | [symbol] | 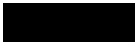 | 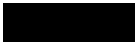 |
| 150 | 399 | A1587986_ESTs | cm50h11.y1 Mus musculus cDNA, 5′ end as: gi-4597033/// /clone=IMAGE:1001733 /clone_end=5′ /gb=A1587986 /gi=4597033 /ug=Mm.31187 /len=330 [gnl\|UG\|Mm#S464186]; m_jongleur(identity): gi_4597033_gb_A1587986.1 _A1587986 | stomach | panc | [symbol] | [symbol] | 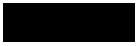 | 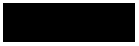 |
| 151 | 380 | A1596550_leukemia/lymphoma_related_factor | Mus musculus leukemia/lymphoma related factor (Lrf), mRNA cr: gi-6754571/// [Mouse_jongleur_201102.3863.C1]; m_jongleur(identity): gi_4605598_gb_A1596550.1 _A1596550 | stomach | panc | [symbol] | [symbol] | 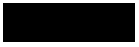 | 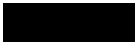 |
| 152 | 424 | AA412912_ESTs | BB404618 Mus musculus cDNA, 3′ end cr: gi-9224014/// /clone=C330037B05 /clone_ebd=3′ /gb=BB404618 /gi=9224014 /ug=Mm.236012 /len=313 [Mouse_jongleur_201102.22092.C1]; m_jongleur(identity): gi_2071468_gb_AA4129121 _AA412912 | stomach | panc | [symbol] | [symbol] | 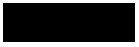 | 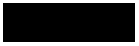 |
| 153 | 340 | AA823475_ESTs | Homo sapiens similar to RNA polymerase 1 transcription factor RRN3 (LOC94431), mRNA si: gi-22067165/// [Human_jongleur_201102.c1.56.singlet] | stomach | panc | [symbol] | [symbol] | 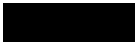 | 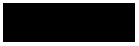 |
| 154 | 415 | A1604192_RIKEN_cDNA_4632433K11_gene | Homo sapiens cDNA FLJ90378 fis, clone NT2RP2004847, weakly similar to ZINC FINGER PROTEIN 135 cr: gi-22760577/// /cds=(5,976) /gb=AK074859 /gi=22760577 /ug=Hs.53996 /len=1987 [Human_jongleur_201102.13667.C1] | stomach | panc | [symbol] | [symbol] | 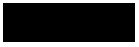 | 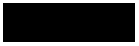 |
| 155 | 412 | AA268472_RIKEN_cDNA_2410003C20_gene | BB667377 Mus musculus cDNA, 3′ end as: gi-16398826/// /clone=C730006115 /clone_end=3′/gb=BB667377 /gi=16398826 /ug=Mm.105955 /len=706 [gnl\|UG\|Mm#S2255490]; m_jongleur(identity): gi_1905243_gb_AA268472.1 _AA268472 | stomach | panc | [symbol] | [symbol] | 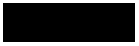 | 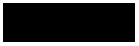 |
| 156 | 382 | A1464600_ESTs | Human phospholipase D mRNA, complete cds | stomach | panc | [symbol] | [symbol] | 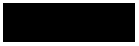 | 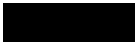 |
| 157 | 314 | AA717080_ESTs | vv70h06.x1 Mus musculus cDNA, 3′ end cr: gi-4613514/// /clone=IMAGE:1227803 /clone_end=3′ /gb=A1604347 /gi=4613514 /ug=Mm.32372 /len=421 [Mouse_jongleur_201102.12391.C1]; m_jongleur(identity): gi_2729354_gb_AA717800.1 _AA717080 | stomach | panc | [symbol] | [symbol] | 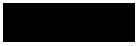 | 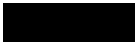 |
| 158 | 370 | AA516997_kinase_suppressor_of_ras | 602849886F1 Homo sapiens cDNA 5′ end cr: gi-14504763/// /clone=IMAGE:4991396 /clone_end=5′ /gb=B1086433 /gi=14504763 /ug=Hs.169061 /len=855. Weakly similar to kinase suppresor of ras [Mus musculus][M.musculus][Human_jongleur_201102.16424.C1] | stomach | panc | [symbol] | [symbol] | 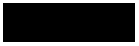 | 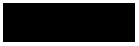 |
| 159 | 409 | AA798385_ESTs | ub61g06.r1 Mus musculus cDNA. 5′ end as: gi-2861340/// /clone=IMAGE:1382266 /clone_end=5′ /gb=AA798385 /gi=2861340 /ug=Mm.25607 /len=473 [gnl\|UG\|Mm#S323719]; m_jongleur(identity): gi_2861340_gb_AA798385.1 _AA798385 | stomach | panc | [symbol] | [symbol] | 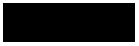 | 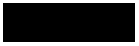 |
| 160 | 432 | A1592746_ESTs | ie33g03.x1 Kaestner ngn3 wt Mus musculus cDNA 3′; est(identity): gi_4601794_gb_A1592746.1_A1592746 | stomach | panc | [symbol] | [symbol] | 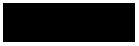 | 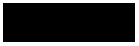 |
| 161 | 312 | AA646049_interleukin_13 | Homo sapiens interleukin 13 (IL13), mRNA cr: gi-4504644/// [Human_jongleur_201102.525.C1] | stomach | panc | [symbol] | [symbol] | 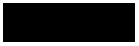 | 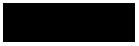 |
| 162 | 331 | AA739464_killer_cell_lectin- like_receptor_subfamily_A_member_9 | Mus musculus killer lectin-like receptor subfamily A, member 21 (Klra21), mRNA cr: gi-21361215/// [Mouse_jongler_201102.3652.C9]; m_jongleur(identity): gi_2775650_gb_AA739464.1 _AA739464 | stomach | panc | [symbol] | [symbol] | 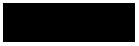 | 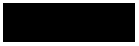 |
| 163 | 334 | A1594144_DNA_segment_Chr_3,_Brigham_&_W omen's_Genetics_0878_expressed | Homo sapiens step II splicing factor SLU7 (SLU7), mRNA cr: gi-20127500/// [Human_jongleur_201102.5919.C1] | stomach | panc | [symbol] | [symbol] | 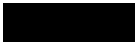 | 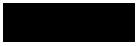 |
| 164 | 386 | A1627071_RIKEN_cDNA_2310058A11_gene | Homo sapiens nuclear phosphoprotein similar to S. cerevisiae PWP1 (PWP1), mRNA cr: gi-5902033/// [Human_jongleur_201102.3438.C1] | stomach | panc | [symbol] | [symbol] | 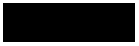 | 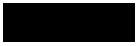 |
| 165 | 419 | A1551677_ESTs | BB032870 Mus musculus cDNA, 3′ end as: gi-15403607/// /clone=5830473M12 /clone_end=3′/gb=BB032870 /gi=15403607 /ug=Mm.30946 /len=660 [gnl\|UG\|Mm#S1035130]; m_jongleur(identity): gi_4484040_gb_A1551677.1 _A1551677 | stomach | panc | [symbol] | [symbol] | 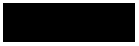 | 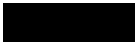 |
| 166 | 319 | AA058211_ESTs | Homo sapiens cDNA FLJ34808 fis, clone MT2ME2006894 cr: gi-21750645/// [Human_jongleur_201102.12659.C1] | stomach | panc | [symbol] | [symbol] | 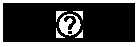 | 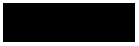 |
| 167 | 342 | AA615213_ESTs,_Moderately_similar_to_unname d_protein_product_[H.sapiens] | Homo sapiens hypothetical protein FLJ110287 (FLJ10287), mRNA cr: gi-11024703/// [Human_jongleur_201102.9152.C1] | stomach | panc | [symbol] | [symbol] | 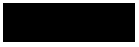 | 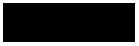 |
| 168 | 313 | AA691755_RIKEN_cDNA_110068E08_gene | Homo sapiens Ku70-binding protein (KUB3) mRNA, partial cds cr: gi-4878033/// /cds=(0,868) /gb=AF078164 /gi=487033 /ug=Hs.61188 /len=1259 [Human_jongleur_201102.13811.C2] | stomach | panc | [symbol] | [symbol] | 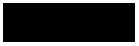 | 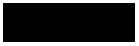 |
| 169 | 406 | AA290276_ESTs | Homo sapiens hypothetical protein FLJ10925 (FLJ10925), mRNA cr: gi-21361725/// [Human_jongleur_201102.10074.C1] | stomach | panc | [symbol] | [symbol] | 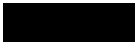 | 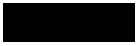 |
| 170 | 426 | A1036904_ESTs_Weakly_similar_in_X- LINKED_LYMPHOCYTE- REGULATED_PROTEIN_PMI_[M.musculus] | Mus musculus adult male testis cDNA, RIKEN full-length enriched library, clone: 4930527E24:similar to XLR-RELATED, MEIOSIS REGULATED) XMR MRNA), full insert sequence cr: gi-12854436/// /cds=(121,588) /gb=AK05913 /gi=12854436 /ug=Mm.48840 /len=733 [Mouse | | | | | | |
| #_jongleur_{201102.16.C230]; m}_jongleur(strong homology): gi_3260595_gb_A1036904.1_A1036904 | stomach | panc | [symbol] | [symbol] | 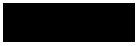 | 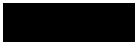 | | | |
| 171 | 279 | A1594170_ESTs | AV323441 Mus musculus cDNA, 3′ end cr: gi-6293358/// /clone=6230409N17 /clone_end=3′/gb=AV323441 /gi=6293358 /ug=Mm.180082 /len=233 [Mouse_jongleur_201102.12236.C1]; m_jongleur(identity): gi_4603218_gb_A15940170.1 _A1594170 | stomach | panc | [symbol] | [symbol] | 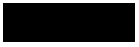 | 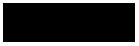 |
| 172 | 392 | AA250545_ESTs_Weakly_similar_to_SFRB_HU MAN_SPLICING_FACTOR_ARGININE/SERINE RICH_II_[H.sapiens] | Homo sapiens hypothetical protein BC013764 (LOC115207), mRNA cr: gi-19923972/// [Human_jongleur_201102.4920.C1] | stomach | panc | [symbol] | [symbol] | 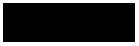 | 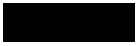 |
| 173 | 326 | A1789680_ESTs | Mus musculus, clone MGC:30823 IMAGE:3995042, mRNA, complete cds cr: gi-21594815/// /cds=(25,387) /gb=BC031793 /gi=21594815 /ug=Mm.220312 /len=3257 [Mouse_jongleur_201102.18084.C1]; m_jongleur(identity): gi_5337396_gb_A1789680.1 _A1789680 | stomach | panc | [symbol] | [symbol] | 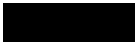 | 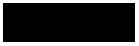 |
| 174 | 346 | AA607043_{—l RIKEN}_cDNA_2400007G07_gene | Mus musculus RIKEN cDNA 2400007G07 gene (2400007G07Rik), mRNA cr: gi-13385367/// [Mouse_jongleur_201102.1540.C1]; m_jongleur(identity): gi_2455936_gb_AA607043.1 _AA607043 | stomach | panc | [symbol] | [symbol] | 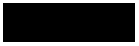 | 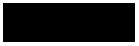 |
| 175 | 361 | AW210270_ESTs,_Weakly_similar_to_T17254_hy pothetical_protein_DKFZp586O1022.1_[H.sapiens ] | 602049523F1 Mus musculus cDNA, 5′end cr: gi-11626724/// /clone=IMAGE:4188868 /clone_end=5′ /gb=BF53943 /gi=11626724 /ug=Mm.184589 /len=1093 [Mouse_jongleur_201102.20725.C1]; m | | | | | | |
| #_jongleur(identity): gi_6516210_gb_AW210270.1 _AW210270 | stomach | panc | [symbol] | [symbol] | 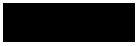 | 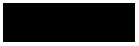 | | | |
| 176 | 332 | AA266331_EST | uu75c10.y1 Mus musculus cDNA, 5′ end as: gi-12651441/// /clone=IMAGE:3382290 /clone_end=5′ /gb=BG148033 /gi=12651441 /ug=Mm.74633 /len=428 [gnl\|UG\|Mm#S1955850]; m_jongleur(identity): gi_1902492_gb_AA266331.1 _AA266331 | stomach | panc | [symbol] | [symbol] | 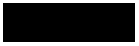 | 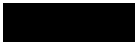 |
| 177 | 417 | AA543950_RIKEN_cDNA_2900073G15_gene | vjb9b10.r1 Knowles Solter mouse blastocyst B1 Mus musculus cDNA clone IMAGE:934267 5′; est(identity): gi_2292427_gb_AA543950.1_AA543950 | stomach | panc | [symbol] | [symbol] | 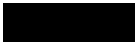 | 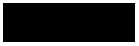 |
| 178 | 362 | A1390609_ESTs | BB123998 Mus musculus cDNA, 3′ end as: gi-16262107/// /clone=9530096B14 /clone_end=3′/gb=BB123998 /gi=16262107 /ug=Mm.32854 /len=672 [gnl\|UG\|Mm#S1123143]; m_jongleur(identity): gi_4216616_gb_A1390609.1 _A1390609 | stomach | panc | [symbol] | [symbol] | 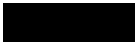 | 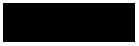 |
| 179 | 400 | AA517691_ESTs | with AGENCOURT_7906247 NIH_MGC_72 Homo sapiens cDNA clone IMAGE:6157921 5′:est(identity): gi_2257215_gb_AA5177691.1 _AA517691 | stomach | panc | [symbol] | [symbol] | 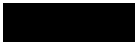 | 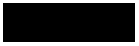 |
| 180 | 433 | AA638805_Public_domain_EST | with Homo sapiens Williams-Beuren syndrome chromosome region 1 (WBSCR1), transcript variant 2, mRNA cr: gi-14702179/// [Human_jongleur_201102.2613.C2]; h_jongleur(identity): gi_2562393_gb_AA638805.1 _AA638805 | stomach | panc | [symbol] | [symbol] | 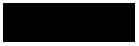 | 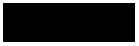 |
| 181 | 301 | AA709576_ESTs | uk38f01.x1 Mus musculus cDNA, 3′ end as: gi-4726650/// /clone=IMAGE:1971289 /clone_end=3′ /gb=A1647922 /gi=4726650 /ug=Mm.80704 /len=583 [gnl\|UG\|Mm#5758354], m_jongleur(identity): gi_2719494_gb_AA709576.1 _AA709576 | stomach | panc | [symbol] | [symbol] | 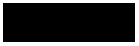 | 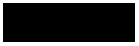 |
| 182 | 315 | AA709668_ESTs | ct35h03.r1 Barstead mouse proximal colon MPLRB6 Mus musculus cDNA clone IMAGE:1165109 5′; est(identity): gi_2719586_gb_AA709668.1 _AA709668 | stomach | panc | [symbol] | [symbol] | 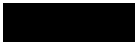 | 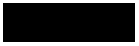 |
| 183 | 296 | A1614679_ESTs | 603312536F1 NCI_CGAP_Mam6 Mus musculus cDNA clone IMAGE:5352609 5′; est(identity): gi_4623846_gb_A1614679.1 _A1614679 | stomach | panc | [symbol] | [symbol] | 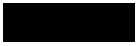 | 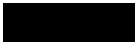 |
| 184 | 367 | A1450469_ESTs | mu72a05.y1 Mus musculus cDNA, 5′ end as: gi-4297220/// /clone=IMAGE:644912 /clone_end=5′/gb=A1450469 /gi=4297220 /ug=Mm.32836 /len=341 [gnl\|UG\|Mm#S433266]; m_jongleur(identity): gi_4297220_gb_A1450469.1 _A1450469 | stomach | panc | [symbol] | [symbol] | 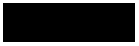 | 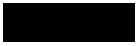 |
| 185 | 328 | AA616077_ESTs | Moderately similar to hypothetical protein FLJ11127 [Homo sapiens] [H.sapiens]cr: gi-11655306/// 602099928F1 Mus musculus cDNA, 5′ end /clone=IMAGE:4219807 /clone_end=5′/gb=BF581594 /gi=11655306 /ug=Mm.24851 /len=1126. [Mouse_jongleur_201102.11302.C1], m_jongleur(identity): gi_2503282_gb | | | | | | |
| #_AA616077.1 _AA616077 | stomach | panc | [symbol] | [symbol] | 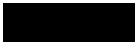 | 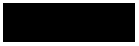 | | | |
| 186 | 351 | AA210560_Mus_musculus_Similar_to_RIKEN_c DNA_1500041N16_gene,_clone_MGC:12066,_mR NA,_complete_cds | Mus musculus RIKEN cDNA 1500041N16 gene (1500041N16Rik), mRNA cr: gi-13385883/// [Mouse_jongleur_201102.167.C4]; m_jongleur(identity)c: gi_1807816 | | | | | | |
| #_gb_AA210560.1 _AA210560 | stomach | panc | [symbol] | [symbol] | 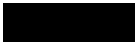 | 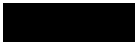 | | | |
| 187 | 371 | A1607675_Mus_musculus_clone_MGC:7865,_mR NA,_complete_cds | Mus musculus ring finger protein 26 (Rnf26), mRNA cr: gi-24415389/// [Mouse_jongleur_201102.9751.C1]; m_jongleur(identity): gi_4616842_gb_A1607676.1 _A1607675 | stomach | panc | [symbol] | [symbol] | 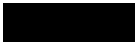 | 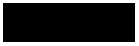 |
| 188 | 394 | AA260596_ESTs | 602390534F1 Mus musculus cDNA, 5′end cr: gi-130533550/// /clone=IMAGE:4502337 /clone_end=5′ /gb=BG293664 /gi=130533550 /ug=Mm.23322 /len=1175 [Mouse_jongleur_201102.11055.C2]; m_jongleur(identity): gi_1901374_gb_AA265096.1 _AA265096 | stomach | panc | [symbol] | [symbol] | 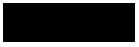 | 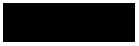 |
| 189 | 404 | AA727914_ESTs | BB083334 Mus musculus cDNA, 3′ end as: gi-1620742/// /clone=9330191N11 /clone_end=3′/gb=BB083334 /gi=16200742 /ug=Mm.45351 /len=673 [gnl\|UG\|Mm#S1125650]; m_jongleur(identity): gi_2745621_gb_AA727914.1 _AA727914 | stomach | panc | [symbol] | [symbol] | 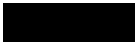 | 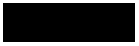 |
| 190 | 423 | AA204045_Public_domain_EST | orth AGENCOURT_8877739 Lupski_sciatic_nerve Homo sapiens cDNA clone IMAGE:6198680 5′; est(identity): gi_1800681_gb_AA204045.1 _A2204045 | stomach | panc | [symbol] | [symbol] | 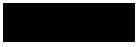 | 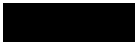 |
| 191 | 435 | AA623587_ESTs | orth df121a08.w1 Morton Fetal Cochlea Homo sapiens cDNA clone IMAGE:2540054 3′; est(identity): gi_2527463_gb_AA623587.1 _AA623587 | stomach | panc | [symbol] | [symbol] | 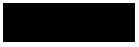 | 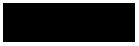 |
| 192 | 410 | AA473386_ESTs | vd164f01.r1 Knowles Solter mouse blastocyst B1 Mus musculus cDNA clone IMAGE:805369 5′; est(identity): gi_2201613_gb_AA473386.1 _AA473386 | stomach | panc | [symbol] | [symbol] | 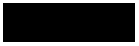 | 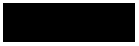 |
| 193 | 318 | AA259871_hypothetical_protein_MNCb-4414 | Homo sapiens mRNA for YEA4 protein (YEA4 gene) cr: gi-20387030/// [Human_jongleur_201102.22053.C1] | stomach | panc | [symbol] | [symbol] | 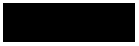 | 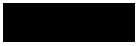 |
| 194 | 425 | AA474374_RIKEN_cDNA_5730408C10_gene | Homo sapiens DKFZP566H074 protein (DKFZP566H073), mRNA cr: gi-14149701/// [Human_jongleur_201102.3235.C1] | stomach | panc | 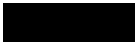 | [symbol] | 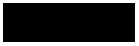 | 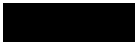 |
| 195 | 427 | AA607267_Public_domain_EST | orth Homo sapiens heterogeneous nuclear ribonucleoprotein U (scafold attachment factor A) (HNRPU), transcript variant 1, mRNA cr: gi-14141162/// [Human_jongleur_201102.5470.C3]; h_jongleur(identity): gi_2456160_gb_AA607267.1 _AA607267 | stomach | panc | [symbol] | [symbol] | 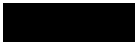 | 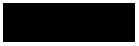 |
| 196 | 369 | AA469648_growth_factor_erv1_(s._cerevisiae)- like_(augmenter_of_liver_regeneration) | Homo sapiens growth factor, augmenter of liver regeneration (ERV1 homolog, S. cerevisiae) (GFER), mRNA cr: gi-4885264/// [Human_jongleur_201102.4053.C3] | stomach | panc | [symbol] | [symbol] | 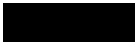 | 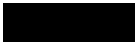 |
| 197 | 393 | A1530661_RIKEN_cDNA_2810426N06_gene | Homo sapiens hypothetical protein MGC26914 (MGC26914), mRNA cr: gi-21699059/// [Human_jongleur_201102.56.C201] | stomach | panc | [symbol] | [symbol] | 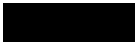 | 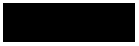 |
| 198 | 358 | A1466825_RIKEN_cDNA_A330103N21_gene | Homo sapiens hypothetical protein MGC18216 (MGC18216), mRNA cr: gi-22748948/// [Human_jongleur_201102.10675.C1] | stomach | panc | [symbol] | [symbol] | 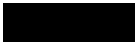 | 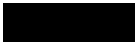 |
| 199 | 421 | AA162023_ESTs | L0901E07-3 Mus musculus cDNA, 3′ end as: gi-1258688/// /clone=L0901E07 /clone_end=3′/gb=BG060262 /gi=12528688 /ug=Mm.171750 /len=128 [gnl\|UG\|Mm#S1912259]; m_jongleur(strong homology): gi_1738329_gb_AA162023.1 _AA162023 | stomach | panc | [symbol] | [symbol] | 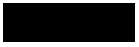 | 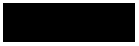 |
| 200 | 436 | AA277192_RIKEN_cDNA_2900075A18_gene | Mus musculus adult male hippocampus cDNA, RIKEN full length enriched library. clone:2900075A18:homolog to CAM-KII INHIBITORY PROTEIN, full insert sequence cr: gi-1281285/// /cds=(31,270) /gb=AK013788 /gi=12851285 /ug=Mm.31199 /len=1222 [Mouse_jongleur_201102.12242.C1]; m_jongleur(identity): gi_1917535_gb | | | | | | |
| #_AA277192.1 _AA277192 | stomach | panc, brain | [symbol] | [symbol] | 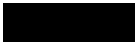 | 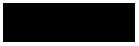 | | | |
| 201 | 291 | A1117905_RIKEN_cDNA_6530406M24_gene | BB486658 Mus musculus cDNA, 3′ end cr: gi-9404267/// /clone=D430037M04 /clone_end=3′/gb=BB486658 /gi=9404267 /ug=Mm.133105 /len=244 [Mouse_jongleur_201102.18536.C1]; m_jongleur(identity): gi_3518229_gb_A1117905.1 _A1117905 | | | | | | |
| #stomach | panc, brain | [symbol] | [symbol] | 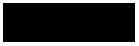 | 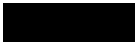 | | | | |
| 202 | 353 | AA684073_ESTs | orth Homo sapiens hypothetical protein FLJ21868 (FLJ21868), mRNA cr: gi-12232446/// [Human_jongleur_201102.3791.C1]; h_jongleur(strong homology): gi_2670659_gb_AA684073.1_AA684073 | stomach | panc, liv | [symbol] | [symbol] | 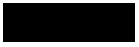 | 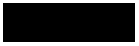 |
| 203 | 295 | A1551472_ESTs | mp88b04.y1 Mus musculus cDNA, 5′ end cr: gi-4483835/// /clone=IMAGE:576271 /clone_end=5′ /gb=A1551472 /gi=4483835 /ug=Mm.229047 /len=404 [Mouse_jongleur_201102.2681.C2]; m_jongleur(identity): gi_4483835_gb_A1551472.1 _A1551472 | stomach | panc, liv | [symbol] | [symbol] | 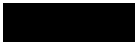 | 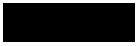 |
| 204 | 194 | 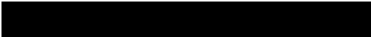 | Mus musculus. Similar to hydroxysteroid 17-beta mdehydrogenase 11, clone MGC:30360 IMAGE:5132342, mRNA, complete cds cr: gi-18043883/// /cds=(49,963) /gb=BC019427 /gi=18043883 /ug=Mm.27300 /len=1719 [Mouse_jongleur_201102.11619.C1]; m_jongleur(density): gi_2892348_gb_AA822480.1 _AA822480 | liver | panc, liv, int | [symbol] | [symbol] | 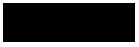 | 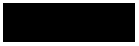 |
| 205 | 76 | 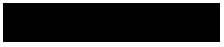 | AV375328 Mus musculus cDNA, 3′ end cr: gi-15412285/// /clone=9130023H10 /clone_end=3′ /gb=AV375328 /gi=15412285 /ug=Mm.27632 /len=641 [Mouse_jongleur_201102.8131.C1]; m_jongleur(identity): gi_2523714_gb_AA619838.1 _AA619838 | bladder | panc, liv, stom, col, brain, blad | [symbol] | [symbol] | [symbol] | 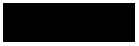 |
| 206 | 48 | 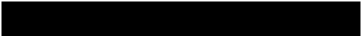 | Homo sapiens cDNA FLJ34661 fis, clone KIDNE2018989, weakly similar to Homo sapiens mRNA for KARP-1 binding protein 2 (KAB2) si: gi-21750471/// [Human_jongleur _201102.c1.16699.singlet] | stomach; brain | panc, stom | [symbol] | [symbol] | 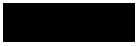 | 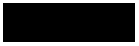 |
| 207 | 87 | 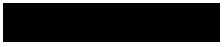 | AGENCOURT_10389813 Mus musculus cDNA, 5′ end as: gi-24032685/// /clone=IMAGE:6594852 /clone_end=5′ /gb=BU847976 /gi=24032685 /ug=Mm.39474 /len=809 [gnl\|UG\|Mm#S2807038]; m_jongleur(strong homology): gi_1796661_gb_AA200457.1_AA200457 | brain | panc, stom | [symbol] | [symbol] | 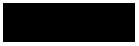 | 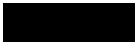 |
| 208 | 92 | 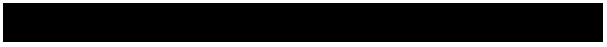 | Mus musculus peptidyl arginine deiminase, type II (Pdi2), mRNA; mus_rs_bna(identity): gi_2078943_gb_AA929696.1 _AA929696 | brain | panc, stom, pl/ut | [symbol] | [symbol] | 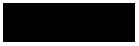 | 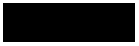 |
| 209 | 336 | 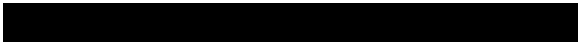 | Mus musculus 18 days embryo whole body cDNA, RIKEN full-length enriched library, clone:1190004M21:hypothetical protein, full insert sequence cr: gi-12835692/// /cds=(1165,1500) /gb=AK004486 /gi=12835692 /ug=Mm.22521 /len=1512 [Mouse_jongleur_201102.10923.C1]; m_jongleur(identity): gi_2346345_gb_AA571416.1 _AA571416 | | | | | | |
| #stomach | panc? | [symbol] | [symbol] | 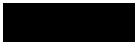 | [symbol] | | | | |
| 210 | 442 | 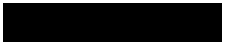 | vp40b11.r1 Mus musculus cDNA, 5′ end as: gi-2893320/// /clone=IMAGE:1079133 /clone_end=5′ /gb=AA823452 /gi=2893320 /ug=Mm.25352 /len=219 [gln\|UG\|Mm#S299539]; m_jongleur(identity): gi_2893320_gb_AA823452.1 _AA823452 | stomach | panc? | [symbol] | [symbol] | 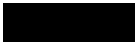 | [symbol] |
| 211 | 356 | 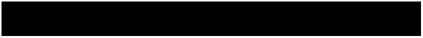 | orth Homo sapiens cDNA FLJ90324 fis, clone NT2RP001817, highly similar to Homo sapiens sirtuin type 1 (SIRT) mRNA si: gi-22760489/// [Human_jongleur_201102.c1.5621.singlet]; h_jongleur(identity): gi_2670375_gb_AA683789.1 _AA683789 | stomach | panc? | [symbol] | [symbol] | 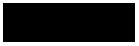 | [symbol] |
| 212 | 384 | 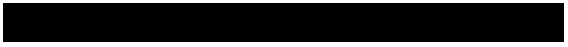 | ve61d02.y1 Beddington mouse embryonic region Mus musculus cDNA clone IMAGE:822627 5′; est(identity): gi_4482024_gb_A1549661.1_A1549661 | stomach | panc? | [symbol] | [symbol] | 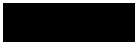 | [symbol] |
| 213 | 408 | 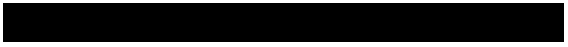 | Homo sapiens nuclear transcription factor, X-box binding 1 (MFX1), transcript variant 1, mRNA cr: gi:22212923/// [Human_jongleur_201102.12184.C2] | stomach | panc? | [symbol] | [symbol] | 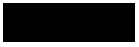 | [symbol] |
| 214 | 396 | 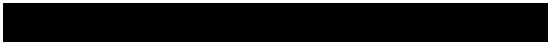 | Mus musculus adult male brain cDNA, RIKEN full-length enriched library. clone:0710001D07:unclassifiable transcript, full insert sequence cr: gi-12833293/// /cds=UNKNOWN /gb=AK002941 /gi=12833293 /ug=Mm.45160 /len=1133 [Mouse_jongleur_201102.13739.C1]; m_jongleur(identity): gi_4596872_gb_A1587825.1 _A1587825 | | | | | | |
| #stomach | panc? | [symbol] | [symbol] | 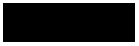 | [symbol] | | | | |
| 215 | 179 | AA013726_cathepsin_J | Human cathepsin L gene, complete cds cr: | intestine | pl/ut | [symbol] | [symbol] | [symbol] | [symbol] |
| | | | gi-809235/// | | | | | | |
| | | | [Human_jongleur_201102.11345.C6] | | | | | | |
| 216 | 255 | 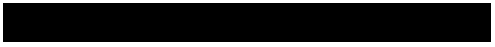 | Mus musculus trophoblast specific protein alpha (Tpbpa), mRNA cr: gi-6678404/// [Mouse_jongleur_201102.3553.C2]; m_jongleur(identity): gi_1834142_gb_AA220024.1 _AA220024 | mammary gland | pl/ut | [symbol] | [symbol] | [symbol] | [symbol] |
| 217 | 81 | AA106071_hemoglobin_beta_adult_major_chain | Human messenger RNA for beta-globin | brain | spl | 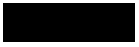 | 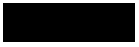 | [symbol] | [symbol] |
| 218 | 82 | AA066763_hemoglobin,_beta_adult_major_chain | Human messenger RNA for beta-globin | brain | spl | 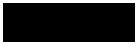 | 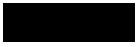 | [symbol] | [symbol] |
| 219 | 83 | W15809_hemoglobin,_beta_adult_major_chain | Human messenger RNA for beta-globin | brain | spl | 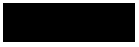 | 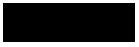 | [symbol] | [symbol] |
| 220 | 85 | A1529513_ESTs,_Moderately_similar_to_F26L_M OUSE_6PF-2-K/FRU-2,6- P2ASE_{1'}LIVER_ISOZYME_[M.musculus] | Moderately similar to F261_Mouse 6- phosphofructo-2-kinase/fructose-2,6- biphosphate 1 (6PF-2-K/Fru-2,6-P2ASE liver isozyme) [Includes: 6-phosphofructo-2-kinase; Fructose-2,6-bisphosphonate] [M.musculus]as gi15714162/// 603360763F1 Mus musculus | | | | | | |
| #cDNA, 5′ end /clone=IMAGE:5368118 /clone_end=5′/gb=B1737149 /gi=15714162 /ug=Mm.132391 /len=896. [gnl\|UG\|Mm#S2184367]; m_jongleur(identity): gi_4443648_gb_A1529513.1 _A1529513 | brain | spl | 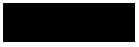 | 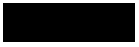 | [symbol] | [symbol] | | | |
| 221 | 96 | A1605734_septin_3 | Homo sapiens septin 3 (SEPT3), transcript variant A, mRNA cr: gi-22035571/// [Human_jongleur_201102.12125.C2] | brain | spl | 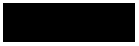 | 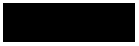 | [symbol] | [symbol] |
| 222 | 97 | AA289586_protein_kinase_C,_beta | Homo sapiens mRNA; cDNA DKFZp76130720 (from clone DKFZp76130720) /gb=AL833252 /gi=21733885 /ug=Hs.349845 /len=3602. Highly similar to KPC1_HUMAN Protein kinase, C, beta-I type (PKC-beta-1) [H.sapiens] cr: gi-21733885/// Homo sapiens mRNA; cDNA DKFZp761H0720 (from clone DKFZp761J0720) /gb=AL833252 /gi=2173885 /ug=Hs.349845 /len=3602. [Human_ | | | | | | |
| #jongleur_201102.20069.C1] | brain | spl, brain | [symbol] | 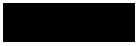 | [symbol] | [symbol] | | | |
| 223 | 114 | A1429054_ESTs | Homo sapiens hypothetical protein | colon | stom | [symbol] | [symbol] | [symbol] | [symbol] |
| | | | FLJ22624 (FLJ22624), mRNA | | | | | | |
| | | | cr: gi-13376190/// | | | | | | |
| | | | [Human_jongleur_201102.4415.C1] | | | | | | |
| 224 | 158 | AA684403_ESTs | AGENCOURT_8840212 Homo sapiens cDNA 5′ end cr: gi-22348912/// /clone=IMAGE:6205296 /clone_end=5′ /gb=BQ933529 /gi=22348912 /ug=Hs.391664 /len=935 Human_jongleur_201102.5512.C2] | intestine | stom | [symbol] | [symbol] | 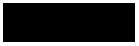 | [symbol] |
| 225 | 191 | AA667301_troponin_1,_cardiac | Human mRNA for cardiac troponin 1 cr: | kidney | stom | [symbol] | [symbol] | [symbol] | [symbol] |
| | | | gi-37427/// | | | | | | |
| | | | [Human_jongleur_201102.9882.C3] | | | | | | |
| 226 | 271 | AA210481_clusterin | Moderately similar to CLUS_HUMAN Clusterin precursor (Complement-associated protein SP-40,40) (Complement cytolysis inhibitor) (CLI) (NA1 and NA2) (Apolipoprotein J) (Apo-J) (TRPM-2) [H.sapiens] as: gi-1615586/// zm89e05.s1 Homo sapiens cDNA 3′ end /clone=IMAGE:545120 /clone_end=3′/gb=AA075716 /gi=1615586 /ug=Hs.394353 /len=626. [gnl\|UG\|Hs#S484558] | | | | | | |
| #pancreas | stom | [symbol] | [symbol] | 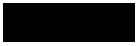 | [symbol] | | | | |
| 227 | 266 | A1120332_RIKEN_cDNA_1810055P16_gene | orth UI-H-BH-abz-i-03-0-UI.s1 Homo sapiens cDNA 3′ end cr: gi-614309/// /clone=IMAGE:2713493 /clone_end=3′/gb=AW136176 /gi=6140309 /ug=Hs.370779 /len=397. Weakly similar to YZA1_HUMAN HYPOTHETICAL PROTEIN [H.sapiens][Human_jongleur_201102.6858.C2]; h_jongleur(identity): gi_3520656 | | | | | | |
| #_gb_A1120332.1 _A1120332 | pancreas | stom | [symbol] | [symbol] | 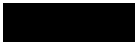 | [symbol] | | | |
| 228 | 267 | AA763351_death-associated_kinase_3 | Homo sapiens death-associated protein Kinase 3 (DAPK3), mRNA cr: gi-4557510/// [Human_jongleur_201102.336.C1] | pancreas | stom | [symbol] | [symbol] | 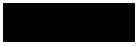 | [symbol] |
| 229 | 261 | 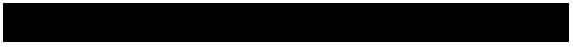 | Homo sapiens, KIAA0186 gene product, clone MGC:13345 IMAGE:4333095, mRNA, complete cds si: gi-15214811/// [Human_jongleur _201102.c1.20423.singlet] | pancreas | stom, breast | [symbol] | [symbol] | 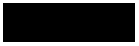 | [symbol] |
| 230 | 91 | AA222952_zuotin_related_factor_2 | Homo sapiens zuotin related factor 1 | brain | | | | | |
| | | | (ZRF1), mRNA cr: gi-22049761/// | | | | | | |
| | | | [Human_jongleur_201102.987.C3] | | | | | | |
| 231 | 99 | A1552958_RIKEN_cDNA_1110003E08_gene | Homo sapiens cDNA FLJ36226 fis, clone | brain | | | | | |
| | | | THYMU2001018, moderately similar to | | | | | | |
| | | | MOB1 PROTEIN si: gi-21752445/// | | | | | | |
| | | | [Human_jongleur | | | | | | |
| | | | _201102.c1.22024.singlet] | | | | | | |
| 232 | 101 | AA117355_RIKEN_cDNA_11900061.01_gene | Moderately similar to S12207 hypothetical | | | | | | |
| | | | protein (B2 element)-mouse [M.musculus] | | | | | | |
| | | | as: gi-20365192/// UI-E-EJ1-aka-c-14-0- | | | | | | |
| | | | U1.r1 Homo sapiens cDNA 5′ end | | | | | | |
| | | | /clone=UI-E-EJI-aka-c-14-0- | | | | | | |
| | | | UI /clone_end=5′ /gb=BQ189641 | | | | | | |
| | | | /gi=20365192 /ug=Hs.373409 /len=566. | | | | | | |
| | | | [gnl\|UG\|Hs#S4404983] | | | | | | |
| 233 | 109 | AA727967_serine_(or_cysteine)_proteinase_inhibit | Homo sapiens serine (or cysteine) | colon | | | | | |
| | | or,_clade_F_(alpha- | proteinase inhibitor, clade F | | | | | | |
| | | 2_antiplasmin,_pigment_epithelium_derived_facto | (alpha-2-antiplastin, pigment epithelium | | | | | | |
| | | r)_member_1 | derived factor), member 1 (SERPINF1), | | | | | | |
| | | | mRNA cr: gi-4505708/// | | | | | | |
| | | | [Human_jongleur_201102.1833.C1] | | | | | | |
| 234 | 125 | AA725966_sphingosine_kinase_1 | Homo sapiens, similar to sphingosine | colon | | | | | |
| | | | kinase, clone MGC:40267 IMAGE:5213270, | | | | | | |
| | | | mRNA, complete cds cr: gi-22539642/// | | | | | | |
| | | | [Human_jongleur_201102.9905.C4] | | | | | | |
| 235 | 130 | AA062408_ESTs,_Moderately_similar_to_proteas | Homo sapiens proteasome (prosome, | colon | | | | | |
| | | ome_inhibitor_hP131_subunit_[H.sapiens] | macropain) inhibitor subunit 1 (P131) | | | | | | |
| | | | (PSMF1), mRNA cr: gi-5803122/// | | | | | | |
| | | | [Human_jongleur_201102.6827.C1] | | | | | | |
| 236 | 138 | AA619953_ESTs,_Weakly_similar_to_T25027_by | Homo sapiens, hypothetical protein similar | colon | | | | | |
| | | pothetcial_protein_T20D3.5_- | to CG7943, clone IMAGE:4827650, mRNA | | | | | | |
| | | _Caenorhabditis_elegans_[C.elegans] | cr: gi-19913535/// | | | | | | |
| | | | [Human_jongleur_201102.1275.C6] | | | | | | |
| 237 | 140 | A1595630_RIKEN_cDNA_4930578106_gene | 603077615F1 Homo sapiens cDNA 5′ end | colon | | | | | |
| | | | cr: gi-15943675/// /clone=IMAGE.5169579 | | | | | | |
| | | | /clone_end=5′ /gb=B1822125 /gi=15943675 | | | | | | |
| | | | /ug=Hs.116415 /len=717 | | | | | | |
| | | | [Human_jongleur_201102.14663.C1] | | | | | | |
| 238 | 142 | AA106834_ESTs,_Weakly_similar_to_EP15_MO | Homo sapiens golgin-245 mRNA, | colon | | | | | |
| | | USE_EPIDERMAL_GROWTH_FACTR_RECEP | complete cds si: gi-1173564/// | | | | | | |
| | | TOR_SUNSTRATE_SUBSTRATE_15_[M.muscul | [Human_jongleur_201102.c1.746.singlet] | | | | | | |
| | | us] | | | | | | | |
| 239 | 145 | AA871395_Mus_musculus,_Similar_to_ectonucleo | Homo sapiens cDNA: FLJ21246 fis, clone | colon | | | | | |
| | | tide_pyrophosphatase/phosphodiesterase_3,_clone_{—} | COL01196, highly similar to AF005632 | | | | | | |
| | | IMAGE:3495326,_mRNA_partial_cds | Homo sapiens phosphodieasterase | | | | | | |
| | | | 1/nucleotide pyrophosphotase beta (PDNP3) | | | | | | |
| | | | mRNA cr: gi-10437313/// /gb=AK024899 | | | | | | |
| | | | /gi=1437313 /ug=Hs.264750 /len=3180. | | | | | | |
| | | | Weakly similar to PC1_HUMAN | | | | | | |
| | | | Ectonucleotide pyrophosp | | | | | | |
| | | | [Human_jongleur_201102.18041.C1] | | | | | | |
| 240 | 162 | AA109528_ESTs,_Weakly_similar_to_CAIC_MO | Mus musculus von Willebrand factor | intestine | | | | | |
| | | USE_COLLAGEN_ALPHA_1(XII)_CHAIN_PRE | A-domain containing 1 (Vwal-pending), | | | | | | |
| | | CURSOR_[M.musculus] | mRNA cr: gi-22296596/// | | | | | | |
| | | | [Mouse_jongleur_201102.9805.C1]; | | | | | | |
| | | | m_jongleur(identity): | | | | | | |
| | | | gi_1661287_gb_AA1095281_AA109528 | | | | | | |
| 241 | 169 | A1552668_ESTs,_Weakly_similar_to_MIA_MOUS | mm03c11.y1 Stratgene mouse kidney | intestine | | | | | |
| | | E_MELANOMNA_DERIVED_GROWTH_REGUL | (#937315) Mus musculus cDNA clone | | | | | | |
| | | ATORY_PROTEIN_PRECURSOR_[M.musculus] | IMAGE:520436 5′; est(identity): | | | | | | |
| | | | gi_4485051_gb_A1552688.1_A1552688 | | | | | | |
| 242 | 210 | AA420359_DNA_segment_Chr_6,_ERATO_Doi_{—} | orth Homo sapiens eukaryotic translation | liver | | | | | |
| | | 109_expressed | termination factor 1 (ETF1), mRNA | | | | | | |
| | | | cr: gi-4759033/// | | | | | | |
| | | | [Human_jongleur_201102.2013.C4]; | | | | | | |
| | | | h_jongleur(identity): | | | | | | |
| | | | gi_2080836_gb_AA420359.1_AA420359 | | | | | | |
| 243 | 236 | AA543982_interleukin_1_receptor,_type_1 | Homo sapiens interleukin 1 receptor, type | liver | | | | | |
| | | | 1 (IL1R1), mRNA cr: gi-4504658/// | | | | | | |
| | | | [Human_jongleur_201102.103C1] | | | | | | |
| 244 | 218 | A1323261_lipase_hepatic | Homo sapiens lipase, hepatic (LIPC), | liver | | | | | |
| | | | mRNA cr: gi-4557722/// | | | | | | |
| | | | [Human_jongleur_201102.432.C1] | | | | | | |
| 245 | 241 | AA274023_apoptosis_inhibitory_6 | Homo sapiens CD5 antigen-like | liver | | | | | |
| | | | (scavenger receptor cysteine rich family) | | | | | | |
| | | | (CD5L), mRNA cr: gi-5174410/// | | | | | | |
| | | | [Human_jongleur_201102.8776.C1] | | | | | | |
| 246 | 242 | AA067863_RIKEN_cDNA_2310057118_gene | Homo sapiens similar to | lung | | | | | |
| | | | evidence:MAS-hypothetical protein-putative | | | | | | |
| | | | (LOC135121), mRNA si: gi-20550015/// | | | | | | |
| | | | [Human_jongleur | | | | | | |
| | | | _201102.c1.17434.singlet] | | | | | | |
| 247 | 40 | AA717025_Mus_musculus_10_day_old_male_pan | Homo sapiens elastase 1, pancreatic | spleen; brain | | | | | |
| | | creas_cDNA,_RIKEN_full- | (ELA1), mRNA cr: gi-22507386/// | | | | | | |
| | | length_enriched_library,_clone: 1810009A17,_full_{—} | [Human_jongleur_201102.9449.C1] | | | | | | |
| | | insert_sequence | | | | | | | |
| 248 | 43 | AA822106_ESTs,_Weakly_similar_to_CORA_HU | Homo sapiens complement component 8, | spleen; liver | | | | | |
| | | MAN_COMPLEMENT_COMPONENT_C8_ALP | alpha polypeptide (C8A), mRNA | | | | | | |
| | | HA_CHAIN_PRECURSOR_[H.sapiens] | cr: gi-4557388/// | | | | | | |
| | | | [Human_jongleur_201102.11044.C1] | | | | | | |
| 249 | 6 | A1447967_ESTs | 603390782F1 Homo sapiens cDNA 5′ end | stomach; | | | | | |
| | | | as; gi-16001577/// /clone=IMAGE:5399756 | brain; | | | | | |
| | | | /clone_end=5′ /gb=B1860842 /gi=16001577 | lacenta/uterus; | | | | | |
| | | | /ug=Hs.112472 /len=917 | mammary | | | | | |
| | | | [gn\|UG\|Hs#S3937223] | gland | | | | | |
| 250 | 53 | A1614738_RIKEN_cDNA_2610301115_gene | Homo sapiens transcription elongation | stomach; | | | | | |
| | | | factor B (S111), polypeptide 1 (15kDa, | brain | | | | | |
| | | | elongin C) (TCEB1), mRNA | | | | | | |
| | | | cr: gi-16933562/// | | | | | | |
| | | | [Human_jongleur_201102.11637.C2] | | | | | | |
| 251 | 55 | A1551391_ESTs,_Weakly_similar_to_FV1_MOUS | orth U1-H-BW1-anl-f-11-0-U1.s1 Homo | stomach; | | | | | |
| | | E_FRIEND_VIRUS_SUSCEPTIBILITY_PROTEI | sapiens cDNA 3′ end cr: gi-11600492/// | brain | | | | | |
| | | N_I_[M.musculus] | /clone=IMAGE:3082892 /clone_end=3′ | | | | | | |
| | | | /gb=BF515225 /gi=11600492 | | | | | | |
| | | | /ug=Hs.221599 /len=439 | | | | | | |
| | | | [Human_jongleur_201102.56.C847]; | | | | | | |
| | | | h_jongleur_(strong homology): | | | | | | |
| | | | gi_4483754_gb_A1551391.1_A1551391 | | | | | | |
| 252 | 56 | AA473684_RIKEN_cDNA_C030002O17_gene | U1-E-E10-aih-g-03-0-U1.s1 Homo sapiens | stomach; | | | | | |
| | | | cDNA 3′ end as: gi-18992463/// | brain | | | | | |
| | | | /clone=U1-E-E10-aih-g-03-0- | | | | | | |
| | | | U1/lone_end=3′ /gb=BM682567 | | | | | | |
| | | | /gi=18992463 /ug=Hs.303527 /len=668 | | | | | | |
| | | | [gnl\|UG\|Hs#S4098264] | | | | | | |
| 253 | 65 | A1549666_ESTs | ve61.g01.y1 Beddington mouse embryonic | stomach; | | | | | |
| | | | region Mus musculus cDNA clone | kidney | | | | | |
| | | | IMAGE:822672 5′; est(identity): | | | | | | |
| | | | gi_4482029_gb_A1549666.1_A1549666 | | | | | | |
| 254 | 3 | A1553220_ESTs | REPEAT orth nr61H04.s1 Homo sapiens | stomach; | | | | | |
| | | | cDNA 3′ end as: gi-2568472/// | lung; bladder; | | | | | |
| | | | /clone=IMAGE:1172503 /clone_end=3′ | placenta/uteru | | | | | |
| | | | /gb=AA643254 /gi=2568472 | s; mammary | | | | | |
| | | | /ug=Hs.407987 /len=552. Weakly similar to | gland | | | | | |
| | | | JAK_MOUSE Tyrosine-protein kinase | | | | | | |
| | | | JAK3 (Janus kinase 3) (JAK-3) | | | | | | |
| | | | [M.musculus] [gnl\|UG\|Hs#S85910]; | | | | | | |
| | | | h_jongleur(identity): | | | | | | |
| | | | gi_44856583_gb_A1553220.1 | | | | | | |
| | | | _A1553220 | | | | | | |
| 255 | 69 | A1549654_programmed_cell_death_10 | orth Homo sapiens programmed cell death | stomach; | | | | | |
| | | | 10 (PDCD10), transcript variant 3, mRNA | mammary | | | | | |
| | | | cr: gi-22538793/// | gland | | | | | |
| | | | [Human_jongleur_201102.12145.C2]; | | | | | | |
| | | | h_jongleur(identity): | | | | | | |
| | | | gi_4482017_gb_A1549654.1 | | | | | | |
| | | | _A1549654 | | | | | | |
| 256 | 73 | AA473562_ESTs,_Moderately_similar_to_alias_D | Homo sapiens deleted in long and | stomach; | | | | | |
| | | LCI_[H.sapiens] | esophogal cancer 1 (DLEC1), transcript | placenta/ | | | | | |
| | | | variant DLEC1-S2, mRNA si: gi-6715592/// | uterus | | | | | |
| | | | [Human_jongleur_201102.c1.6757.singlet] | | | | | | |
| 257 | 294 | AA655910_mast_cell_protease_2 | orth Human mast cell chymase gene, | stomach | | | | | |
| | | | complete cds cr: gi-180541/// | | | | | | |
| | | | [Human_jongleur_201102.12216.C2]; | | | | | | |
| | | | h_jongleur(identity): | | | | | | |
| | | | gi_2592064_gb_AA655910.1_AA655910 | | | | | | |
| 258 | 297 | A1552048_ESTs | Homo sapiens hypothetical protein | stomach | | | | | |
| | | | MGC2628 (MGC2628), mRNA | | | | | | |
| | | | cr: gi-13129063/// | | | | | | |
| | | | [Human_jongleur_201102.3501.C1] | | | | | | |
| 259 | 390 | AA517193_unc119_homolog_(C._elegans) | Homo sapiens unc-119 homolog (C. | stomach | | | | | |
| | | | elegans) (UNC119), transcript variant 1, | | | | | | |
| | | | mRNA cr: gi-16936537/// | | | | | | |
| | | | [Human_jongleur_201102.4091.C1] | | | | | | |
| 260 | 307 | AA211954_RIKEN_cDNA_4930511A05_gene | Homo sapiens hypothetical SBB103 | stomach | | | | | |
| | | | protein (SBB103), mRNA cr: gi-5032070/// | | | | | | |
| | | | [Human_jongleur_201102.6023.C1] | | | | | | |
| 261 | 308 | AA530026_ESTs | Human phosphoglycerate mutase 2 | stomach | | | | | |
| | | | (muscle) specific isozyme) (PGAM2) gene, | | | | | | |
| | | | 5′; end si: gi-189869/// | | | | | | |
| | | | [Human_jongleur_201102.c125.singlet] | | | | | | |
| 262 | 311 | AA438040_Public_domain_EST | Homo sapiens KIAA0877 protein | stomach | | | | | |
| | | | (KIAA877), mRNA si: gi-20541808/// | | | | | | |
| | | | [Human_jongleur_201102.c1.56.singlet] | | | | | | |
| 263 | 317 | AA470294_DNA_segment,_Chr_2,_ERATO_{—4}Doi_{—} | Homo sapiens gamma tubulin ring | stomach | | | | | |
| | | 435,_expressed | complex protein (76p gene) (76P), | | | | | | |
| | | | mRNA cr: gi-7656840/// | | | | | | |
| | | | [Human_jongleur_201102.1500.C1] | | | | | | |
| 264 | 325 | AA210559_ESTs,_Weakly_similar_to_T34029_hy | Homo sapiens oxysterol binding protein- | stomach | | | | | |
| | | pothetical_protein_C32F10.1_ | like 11 (OSBPL11), mRNA | | | | | | |
| | | _Caenorhabditis_elegans_[C.elegans] | cr: gi-23111058/// | | | | | | |
| | | | [Human_jongleur_201102.12127.C1] | | | | | | |
| 265 | 327 | A1553356_ESTs,_Moderately_similar_to_T00075_{—} | Homo sapiens pogo transposable element | stomach | | | | | |
| | | hypothetical_protein_KIAA0461_[H.sapiens] | with ZNF domain (POGZ), transcript | | | | | | |
| | | | variant 1, mRNA cr: gi-2207468/// | | | | | | |
| | | | [Human_jongleur_201102.11858.C1] | | | | | | |
| 266 | 330 | AA254875_ESTs | AV378849 RIKEN full-length enriched, | stomach | | | | | |
| | | | adult male cecum Mus musculus cDNA | | | | | | |
| | | | clone 9130604316 3′; est(identity): | | | | | | |
| | | | gi_1889462_gb_AA254875.1_AA254875 | | | | | | |
| 267 | 341 | AA470824_serine/threonine_kinase_5 | Homo sapiens serine/threonine kinase 12 | stomach | | | | | |
| | | | (SYK12), mRNA cr: gi-4759177/// | | | | | | |
| | | | [Human_jongleur_201102.967.C1] | | | | | | |
| 268 | 345 | A1594129_Mus_musculus_clone_INAGE:349441 | Homo sapiens, Similar to like- | stomach | | | | | |
| | | 1,_mRNA_partial,_cds | glycosyltransferase, clone MGC:33333 | | | | | | |
| | | | IMAGE:4821103, mRNA, complete cds | | | | | | |
| | | | cr: gi-22713409/// | | | | | | |
| | | | [Human_jongleur_201102.10581.C2] | | | | | | |
| 269 | 348 | AA450453_Mus_musculus,_Similar_to_hypothetic | Homo sapiens hypothetical protein | stomach | | | | | |
| | | al_protein,_clone_MGC:7581,_mRNA,_complete_{—} | CL25022 (CL25022), mRNA | | | | | | |
| | | cds | cr: gi-7661547/// | | | | | | |
| | | | [Human_jongleur_201102.9081.C1] | | | | | | |
| 270 | 352 | A1550569_ESTs | BB053703 RIKEN full-length enriched, | stomach | | | | | |
| | | | 12 days embryo male wolffan duct Mus | | | | | | |
| | | | musculus cDNA clone 6720465D24 3′; | | | | | | |
| | | | est(identity): | | | | | | |
| | | | gi_4482932_gb_A1550569.1_A1550569 | | | | | | |
| 271 | 360 | AA464160_ESTs | Homo sapiens hypothetical protein | stomach | | | | | |
| | | | DKFZp564K0822 (DKFZP564K0822), | | | | | | |
| | | | mRNA cr: gi-13540577/// | | | | | | |
| | | | [Human_jongleur_201102.5608.C1] | | | | | | |
| 272 | 364 | AA212102_ESTs | AGENCORUT_7896003 Homo sapiens | stomach | | | | | |
| | | | cDNA 5′ end cr: gi-21179761/// | | | | | | |
| | | | /clone=IMAGE:6156113 /clone_end=5′ | | | | | | |
| | | | /gb=BQ440685 /gi=21179761 | | | | | | |
| | | | /ug=Hs.191045 /len=883 | | | | | | |
| | | | [Human_jongleur_201102.16845.C1] | | | | | | |
| 273 | 373 | AA624604_Public_domain_EST | Homo sapiens heterogeneous nuclear | stomach | | | | | |
| | | | ribonucleoprotein L (HNRPL), mRNA | | | | | | |
| | | | cr: gi-4557644/// | | | | | | |
| | | | [Human_jongleur_201102.4589.C4] | | | | | | |
| 274 | 375 | AA413026_Public_domain_EST | Homo sapiens GDP dissociation inhibitor | stomach | | | | | |
| | | | 2 (GD12), mRNA cr: gi-6598322/// | | | | | | |
| | | | [Human_jongleur_201102.744.C2] | | | | | | |
| 275 | 378 | AA105367_period_homolog_3_(Drosophila) | Homo sapiens mRNA for period | stomach | | | | | |
| | | | (Drosophila) homolog 3 hPER3, complete | | | | | | |
| | | | cds cr: gi-13160924/// | | | | | | |
| | | | [Human_jongleur_201102.2143.C5] | | | | | | |
| 276 | 381 | AA209628_ESTs,_Highly_similar_to_T08741_hyp | Homo sapiens cDNA FLJ10331 fis, clone | stomach | | | | | |
| | | othetical_protein_DKFZp586G0518.1_[H.sapiens] | NT2RM2000635, highly similar to Homo | | | | | | |
| | | | sapiens mRNA for KIAA0729 | | | | | | |
| | | | protein cr: gi-7022294/// | | | | | | |
| | | | [Human_jongleur_201102.3321.C2] | | | | | | |
| 277 | 385 | AA265101_RIKEN_cDNA_1600021C16_gene | orth Homo sapiens hypothetical protein | stomach | | | | | |
| | | | FLJ10997 (FLJ10997), mRNA | | | | | | |
| | | | cr: gi-8922809/// | | | | | | |
| | | | [Human_jongleur_201102.2733.C1]; | | | | | | |
| | | | h_jongleur(strong homology): | | | | | | |
| | | | gi_1901379_gb_AA265101.1_AA265101 | | | | | | |
| 278 | 395 | AA529248_Mus_musculus,_Similar_to_phosphor | orth Homo sapiens phosphoprotein | stomach | | | | | |
| | | otein_regulated_by_mitogenic_pathways,_clone_M | regulated by mitogenic pathways (C8FW), | | | | | | |
| | | GC:11752,_mRNA,_complete_cds | mRNA cr: gi-13399327/// | | | | | | |
| | | | [Human_jongleur_201102.3199.C1]; | | | | | | |
| | | | h_jongleur(strong homology): | | | | | | |
| | | | gi_2271954_gb_AA529248.1_AA529248 | | | | | | |
| 279 | 397 | AA265337_deoxyguanosine_kinase | Homo sapiens deoxyguanosine kinase | stomach | | | | | |
| | | | (DGUOK), transcript variant 1, nuclear | | | | | | |
| | | | gene encoding mitochondrial protein, | | | | | | |
| | | | mRNA, cr: gi-18326966/// | | | | | | |
| | | | [Human_jongleur_201102.4322.C2] | | | | | | |
| 280 | 411 | A1644723_ESTs,_Weakly_similar_to_JH0494_alp | qv66d05.x1 Homo sapiens cDNA 3′ end | stomach | | | | | |
| | | ha-1-antichymotrypsin-like_protein_EB2217_- | cr: gi-3897024/// /clone=UIMAGE:1986537 | | | | | | |
| | | _mouse_[M.musculus] | /clone_end=3′ /gb=A1274750 /gi=3897024 | | | | | | |
| | | | /ug=Hs.145890 /len=434. Weakly similar to | | | | | | |
| | | | KAIN_HUMAN Kallistatin precursor | | | | | | |
| | | | (Kallikrein inhibitor) (Protease inhibitor 4) | | | | | | |
| | | | [H.sapiens] | | | | | | |
| | | | [Human_jongleur_201102.15782.C1] | | | | | | |
| 281 | 413 | A1551943_ESTs,_Weakly_similar_to_JC2378_acet | xj05c01.x1 Homo sapiens cDNA 3′ end | stomach | | | | | |
| | | yl-CoA_C-acetyltransferase_[H.sapiens] | as: gi-6438834/// /clone=IMAGE:2656320 | | | | | | |
| | | | /clone_end=3′ /gb=AW172886 /gi=6438834 | | | | | | |
| | | | /ug=Hs.277923 /len=250 | | | | | | |
| | | | [gnl\|UG\|Hs#S1666742] | | | | | | |
| 282 | 416 | A1559155_RIKEN_cDNA_6330415E02_gene | orth Homo sapiens LOC219701 | stomach | | | | | |
| | | | (LOC219701), mRNA si: gi-20545460/// | | | | | | |
| | | | [Human_jongleur | | | | | | |
| | | | _201102.c1.12722.singlet]; | | | | | | |
| | | | h_jongleur(identity): | | | | | | |
| | | | gi_4484318_gb_A1551955.1 | | | | | | |
| | | | _A1551955 | | | | | | |
| 283 | 438 | AA210502_Public_domain_EST | Homo sapiens HSPC150 protein similar to | stomach | | | | | |
| | | | ubiquitin-conjugating enzyme (HSPC150), | | | | | | |
| | | | mRNA cr: gi-7661807/// | | | | | | |
| | | | [Human_jongleur_201102.6192.C1] | | | | | | |
| 284 | 437 | A1550200_SEC61,_alpha_subunit_2_S._cerevisia | orth UI-H-DF1-aug-m-18-0-UI.s1 | | | | | | |
| | | e) | NCI_CGAP_DF1 Homo sapiens cDNA | | | | | | |
| | | | clone UI-H-DFI-aug-m-18-0-UI 3′; | | | | | | |
| | | | est(identity): | | | | | | |
| | | | gi_4482563_gb_A1550200.1_A1550200 | | | | | | |
| 285 | 438 | A1549694_ESTs,_Highly_similar_to_P300_HUMA | orth UI-H-EH-ayy-i-09-0-UI.s1 | stomach | | | | | |
| | | N_EIA- | NCI_CGAP_EH Homo sapiens cDNA | | | | | | |
| | | ASSOCIATED_PROTEIN_P300_[H.sapiens] | clone UI-H-EH-ayy-i-09-0-UI 3′; | | | | | | |
| | | | est(identity): | | | | | | |
| | | | gi_4482057_gb_A1549694.1_A1549694 | | | | | | |
| 286 | 440 | A1552902_RIKEN_cDNA_1700049101_gene | orth Homo sapiens hypothetical protein | stomach | | | | | |
| | | | DKFZp7621194 (DKFZp7621194), mRNA | | | | | | |
| | | | cr: gi-22748822/// | | | | | | |
| | | | [Human_jongleur_201102.10630.C1]; | | | | | | |
| | | | h_jongleur(identity): | | | | | | |
| | | | gi_4485265_gb_A1552902.1 | | | | | | |
| | | | _A1552902 | | | | | | |
| 287 | 9 | AA389264_ESTs | BB034567 Mus musculus cDNA, 3′ end | stomach | | | | | |
| | | | cr: gi-15403646/// /clone=5830499F16 | | | | | | |
| | | | /clone_end=3′ /gb=BB034567 /gi=15403646 | | | | | | |
| | | | /ug=Mm.27986 /len=699 | | | | | | |
| | | | [Mouse_jongleur_201102.12991.C1]; | | | | | | |
| | | | m_jongleur(identity): | | | | | | |
| | | | gi_2042219_gb_AA289264.1_AA389264 | | | | | | |
| 288 | 443 | AA638776_matrix_metalloproteinase_12 | Homo sapiens matrix metalloproteinase 12 | stomach | | | | | |
| | | | (macrophage elastase) (MMP12), mRNA | | | | | | |
| | | | cr: gi-4505206/// | | | | | | |
| | | | [Human_jongleur_201102.11395.C1] | | | | | | |
| 289 | 445 | AA606582_Public_domain_EST | UI-M-DJI-bty-f-10-0-UI.s1 | stomach | | | | | |
| | | | NH_BMAP_DJI Mus musculus cDNA | | | | | | |
| | | | clone UI-M-DJI-bty-f-10-0-UI 3′; | | | | | | |
| | | | est(identity): | | | | | | |
| | | | gi_2455516_gb_AA606852.1 | | | | | | |
| | | | _AA606582 | | | | | | |

| | | | | | | |
|---|---|---|---|---|---|---|
| 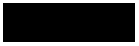 | 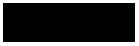 | 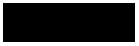 | 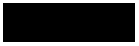 | 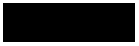 | 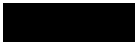 | 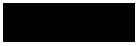 |

| | | (−) | (+) | (−) | (+) | (−) | (+) | (−) | (+) | (−) | (+) | (−) | (+) | (−) | (+) | |
|---|---|---|---|---|---|---|---|---|---|---|---|---|---|---|---|---|
| SEQ | | [symbol] | [symbol] | [symbol] | [symbol] | [symbol] | [symbol] | [symbol] | [symbol] | [symbol] | [symbol] | [symbol] | [symbol] | [symbol] | [symbol] | |
| ID | | [symbol] | [symbol] | [symbol] | [symbol] | [symbol] | [symbol] | [symbol] | [symbol] | [symbol] | [symbol] | [symbol] | [symbol] | [symbol] | [symbol] | |
| NO | ID: | [symbol] | [symbol] | [symbol] | [symbol] | [symbol] | [symbol] | [symbol] | [symbol] | [symbol] | [symbol] | [symbol] | [symbol] | [symbol] | [symbol] | |
| 1 | 161 | [symbol] | [symbol] | [symbol] | [symbol] | 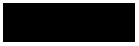 | [symbol] | 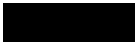 | [symbol] | [symbol] | [symbol] | [symbol] | [symbol] | 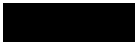 | 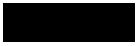 | |
| 2 | 47 | [symbol] | [symbol] | 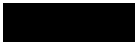 | [symbol] | [symbol] | [symbol] | [symbol] | [symbol] | [symbol] | [symbol] | [symbol] | [symbol] | 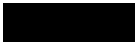 | 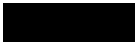 | |
| 3 | 252 | [symbol] | [symbol] | [symbol] | [symbol] | [symbol] | [symbol] | [symbol] | [symbol] | [symbol] | [symbol] | [symbol] | [symbol] | 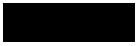 | 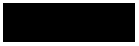 | |
| 4 | 98 | [symbol] | [symbol] | [symbol] | [symbol] | [symbol] | [symbol] | [symbol] | [symbol] | [symbol] | [symbol] | 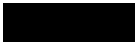 | 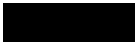 | [symbol] | [symbol] | |
| 5 | 94 | [symbol] | [symbol] | [symbol] | [symbol] | [symbol] | [symbol] | [symbol] | [symbol] | [symbol] | [symbol] | 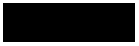 | 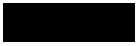 | [symbol] | [symbol] | |
| 6 | 104 | [symbol] | [symbol] | [symbol] | [symbol] | [symbol] | [symbol] | [symbol] | [symbol] | [symbol] | [symbol] | 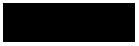 | 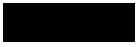 | [symbol] | [symbol] | |
| 7 | 122 | [symbol] | [symbol] | [symbol] | [symbol] | [symbol] | [symbol] | 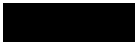 | [symbol] | [symbol] | [symbol] | [symbol] | 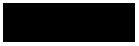 | [symbol] | [symbol] | |
| 8 | 115 | 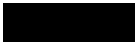 | [symbol] | [symbol] | [symbol] | 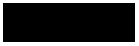 | [symbol] | 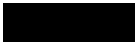 | [symbol] | [symbol] | [symbol] | [symbol] | 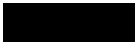 | [symbol] | [symbol] | |
| 9 | 118 | 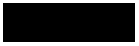 | [symbol] | [symbol] | [symbol] | [symbol] | [symbol] | 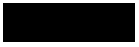 | [symbol] | [symbol] | [symbol] | [symbol] | 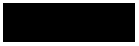 | [symbol] | [symbol] | |
| 10 | 116 | [symbol] | [symbol] | [symbol] | [symbol] | [symbol] | [symbol] | 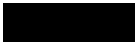 | [symbol] | [symbol] | [symbol] | [symbol] | [symbol] | [symbol] | [symbol] | |
| 11 | 128 | [symbol] | [symbol] | [symbol] | [symbol] | [symbol] | [symbol] | 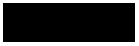 | [symbol] | [symbol] | [symbol] | [symbol] | 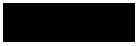 | [symbol] | [symbol] | |
| 12 | 189 | [symbol] | [symbol] | [symbol] | [symbol] | [symbol] | [symbol] | [symbol] | [symbol] | [symbol] | [symbol] | 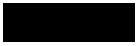 | 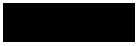 | [symbol] | [symbol] | |
| 13 | 235 | [symbol] | [symbol] | [symbol] | [symbol] | [symbol] | [symbol] | [symbol] | [symbol] | [symbol] | [symbol] | 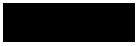 | 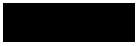 | [symbol] | [symbol] | |
| 14 | 269 | [symbol] | [symbol] | [symbol] | [symbol] | [symbol] | [symbol] | [symbol] | [symbol] | [symbol] | [symbol] | 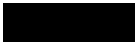 | 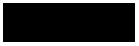 | [symbol] | [symbol] | |
| 15 | 262 | [symbol] | [symbol] | [symbol] | [symbol] | [symbol] | [symbol] | [symbol] | [symbol] | [symbol] | [symbol] | 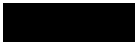 | 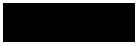 | [symbol] | [symbol] | |
| 16 | 263 | [symbol] | [symbol] | [symbol] | [symbol] | [symbol] | [symbol] | [symbol] | [symbol] | [symbol] | [symbol] | 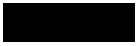 | 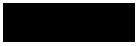 | [symbol] | [symbol] | |
| 17 | 50 | [symbol] | [symbol] | 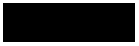 | [symbol] | [symbol] | [symbol] | [symbol] | [symbol] | [symbol] | [symbol] | 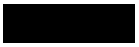 | 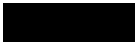 | [symbol] | [symbol] | |
| 18 | 58 | [symbol] | [symbol] | 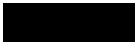 | [symbol] | [symbol] | [symbol] | 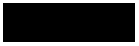 | [symbol] | [symbol] | [symbol] | 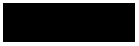 | 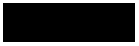 | [symbol] | [symbol] | |
| 19 | 337 | [symbol] | [symbol] | [symbol] | [symbol] | [symbol] | [symbol] | [symbol] | [symbol] | [symbol] | [symbol] | 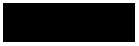 | 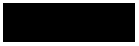 | [symbol] | [symbol] | |
| 20 | 333 | [symbol] | [symbol] | [symbol] | [symbol] | [symbol] | [symbol] | [symbol] | [symbol] | [symbol] | [symbol] | 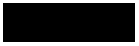 | 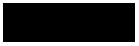 | [symbol] | [symbol] | |
| 21 | 357 | [symbol] | [symbol] | 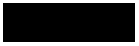 | [symbol] | [symbol] | [symbol] | [symbol] | [symbol] | [symbol] | [symbol] | 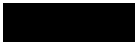 | 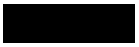 | [symbol] | [symbol] | |
| 22 | 309 | [symbol] | [symbol] | [symbol] | [symbol] | [symbol] | [symbol] | 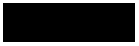 | [symbol] | [symbol] | [symbol] | 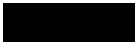 | 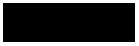 | [symbol] | [symbol] | |
| 23 | [symbol] | [symbol] | 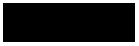 | [symbol] | [symbol] | [symbol] | [symbol] | [symbol] | [symbol] | [symbol] | 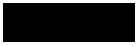 | 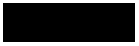 | 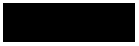 | 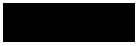 | | |
| 24 | 155 | [symbol] | [symbol] | [symbol] | [symbol] | [symbol] | [symbol] | [symbol] | [symbol] | [symbol] | [symbol] | 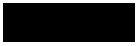 | 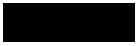 | [symbol] | [symbol] | |
| 25 | 292 | 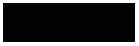 | [symbol] | 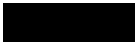 | [symbol] | 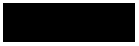 | [symbol] | 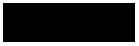 | [symbol] | [symbol] | [symbol] | 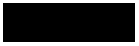 | 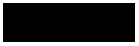 | [symbol] | [symbol] | |
| 26 | 120 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 27 | 202 | 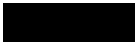 | [symbol] | [symbol] | [symbol] | [symbol] | [symbol] | [symbol] | [symbol] | [symbol] | [symbol] | [symbol] | [symbol] | [symbol] | [symbol] | |
| 28 | 196 | 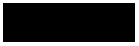 | [symbol] | [symbol] | [symbol] | [symbol] | [symbol] | [symbol] | [symbol] | [symbol] | [symbol] | [symbol] | [symbol] | [symbol] | [symbol] | |
| 29 | 253 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 30 | 250 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 31 | 152 | [symbol] | [symbol] | [symbol] | [symbol] | 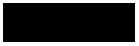 | [symbol] | 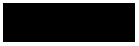 | 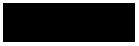 | 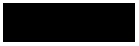 | 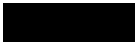 | [symbol] | [symbol] | 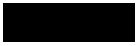 | [symbol] | |
| 32 | 181 | [symbol] | [symbol] | [symbol] | [symbol] | 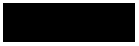 | [symbol] | 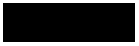 | 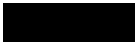 | [symbol] | [symbol] | [symbol] | [symbol] | [symbol] | [symbol] | |
| 33 | 107 | [symbol] | [symbol] | [symbol] | [symbol] | 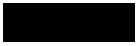 | 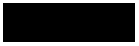 | 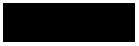 | [symbol] | [symbol] | [symbol] | [symbol] | [symbol] | [symbol] | [symbol] | |
| 34 | 129 | [symbol] | [symbol] | [symbol] | [symbol] | 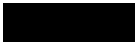 | 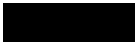 | 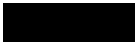 | [symbol] | [symbol] | [symbol] | [symbol] | [symbol] | [symbol] | [symbol] | |
| 35 | 160 | [symbol] | [symbol] | [symbol] | [symbol] | 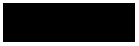 | 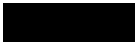 | 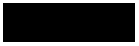 | [symbol] | 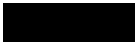 | [symbol] | [symbol] | [symbol] | [symbol] | [symbol] | |
| 36 | 17 | [symbol] | [symbol] | [symbol] | [symbol] | 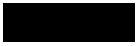 | 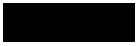 | 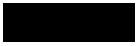 | 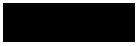 | [symbol] | [symbol] | [symbol] | [symbol] | [symbol] | [symbol] | |
| 37 | 19 | [symbol] | [symbol] | [symbol] | [symbol] | 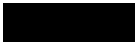 | 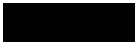 | 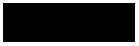 | 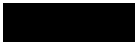 | [symbol] | [symbol] | [symbol] | [symbol] | [symbol] | [symbol] | |
| 38 | 136 | [symbol] | [symbol] | [symbol] | [symbol] | 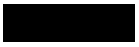 | 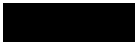 | 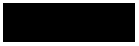 | 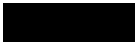 | [symbol] | [symbol] | [symbol] | [symbol] | [symbol] | [symbol] | |
| 39 | 112 | [symbol] | [symbol] | [symbol] | [symbol] | 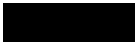 | 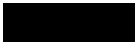 | 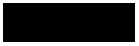 | 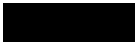 | [symbol] | [symbol] | [symbol] | [symbol] | [symbol] | [symbol] | |
| 40 | 25 | [symbol] | [symbol] | [symbol] | [symbol] | 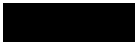 | 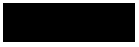 | 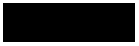 | 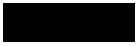 | [symbol] | [symbol] | [symbol] | [symbol] | [symbol] | [symbol] | |
| 41 | 26 | [symbol] | [symbol] | [symbol] | [symbol] | 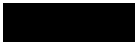 | 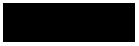 | 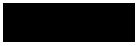 | 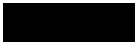 | [symbol] | [symbol] | [symbol] | [symbol] | [symbol] | [symbol] | |
| 42 | 27 | [symbol] | [symbol] | [symbol] | [symbol] | 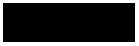 | 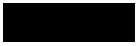 | 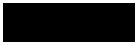 | 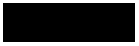 | [symbol] | [symbol] | [symbol] | [symbol] | [symbol] | [symbol] | |
| 43 | 163 | [symbol] | [symbol] | [symbol] | [symbol] | 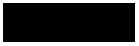 | 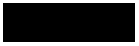 | 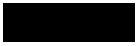 | 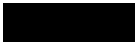 | [symbol] | [symbol] | [symbol] | [symbol] | [symbol] | [symbol] | |
| 44 | 177 | [symbol] | [symbol] | [symbol] | [symbol] | 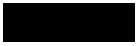 | 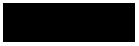 | 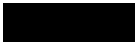 | 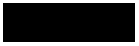 | [symbol] | [symbol] | [symbol] | [symbol] | [symbol] | [symbol] | |
| 45 | 167 | [symbol] | [symbol] | [symbol] | [symbol] | 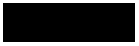 | 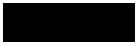 | 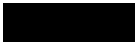 | 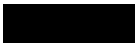 | [symbol] | [symbol] | [symbol] | [symbol] | [symbol] | [symbol] | |
| 46 | 78 | [symbol] | [symbol] | [symbol] | [symbol] | 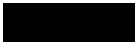 | 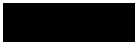 | 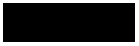 | 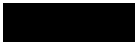 | [symbol] | [symbol] | [symbol] | [symbol] | [symbol] | [symbol] | |
| 47 | 174 | [symbol] | [symbol] | [symbol] | [symbol] | 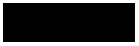 | 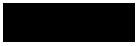 | 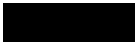 | 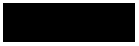 | [symbol] | [symbol] | [symbol] | [symbol] | [symbol] | [symbol] | |
| 48 | 38 | [symbol] | [symbol] | [symbol] | [symbol] | 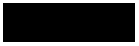 | 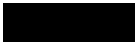 | 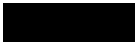 | 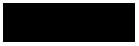 | [symbol] | [symbol] | [symbol] | [symbol] | [symbol] | [symbol] | |
| 49 | 60 | [symbol] | [symbol] | [symbol] | [symbol] | 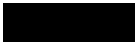 | 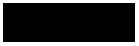 | 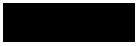 | 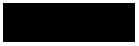 | [symbol] | [symbol] | [symbol] | [symbol] | [symbol] | [symbol] | |
| 50 | 62 | [symbol] | [symbol] | [symbol] | [symbol] | 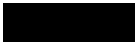 | 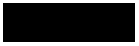 | 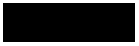 | 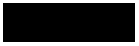 | [symbol] | [symbol] | [symbol] | [symbol] | [symbol] | [symbol] | |
| 51 | 106 | [symbol] | [symbol] | [symbol] | [symbol] | [symbol] | 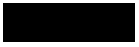 | [symbol] | [symbol] | [symbol] | [symbol] | [symbol] | 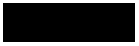 | [symbol] | [symbol] | [symbol] |
| 52 | 170 | [symbol] | [symbol] | [symbol] | [symbol] | 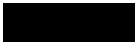 | [symbol] | 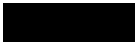 | [symbol] | [symbol] | [symbol] | [symbol] | [symbol] | [symbol] | [symbol] | |
| 53 | 180 | [symbol] | [symbol] | [symbol] | [symbol] | 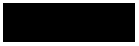 | [symbol] | 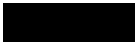 | [symbol] | [symbol] | [symbol] | [symbol] | [symbol] | [symbol] | [symbol] | |
| 54 | 29 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 55 | 187 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 56 | 219 | 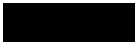 | [symbol] | [symbol] | [symbol] | [symbol] | [symbol] | [symbol] | [symbol] | [symbol] | [symbol] | [symbol] | [symbol] | [symbol] | [symbol] | |
| 57 | 222 | 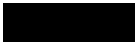 | [symbol] | [symbol] | [symbol] | [symbol] | [symbol] | [symbol] | [symbol] | [symbol] | [symbol] | [symbol] | [symbol] | [symbol] | [symbol] | |
| 58 | 30 | 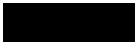 | 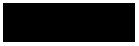 | [symbol] | [symbol] | [symbol] | [symbol] | [symbol] | [symbol] | [symbol] | [symbol] | [symbol] | [symbol] | [symbol] | [symbol] | |
| 59 | 213 | 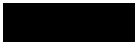 | 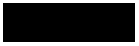 | [symbol] | [symbol] | [symbol] | [symbol] | [symbol] | [symbol] | [symbol] | [symbol] | [symbol] | [symbol] | [symbol] | [symbol] | |
| 60 | 267 | 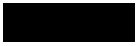 | 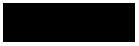 | [symbol] | [symbol] | [symbol] | [symbol] | [symbol] | [symbol] | [symbol] | [symbol] | [symbol] | [symbol] | [symbol] | [symbol] | |
| 61 | 205 | 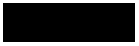 | 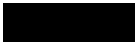 | [symbol] | [symbol] | [symbol] | [symbol] | [symbol] | [symbol] | [symbol] | [symbol] | [symbol] | [symbol] | [symbol] | [symbol] | |
| 62 | 237 | 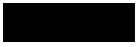 | 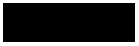 | [symbol] | [symbol] | [symbol] | [symbol] | [symbol] | [symbol] | [symbol] | [symbol] | [symbol] | [symbol] | [symbol] | [symbol] | |
| 63 | 211 | 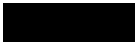 | 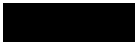 | [symbol] | [symbol] | [symbol] | [symbol] | [symbol] | [symbol] | [symbol] | [symbol] | [symbol] | [symbol] | [symbol] | [symbol] | |
| 64 | 233 | 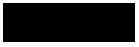 | 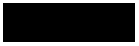 | [symbol] | [symbol] | [symbol] | [symbol] | [symbol] | [symbol] | [symbol] | [symbol] | [symbol] | [symbol] | [symbol] | [symbol] | |
| 65 | 230 | 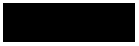 | 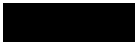 | [symbol] | [symbol] | [symbol] | [symbol] | [symbol] | [symbol] | [symbol] | [symbol] | [symbol] | [symbol] | [symbol] | [symbol] | |
| 66 | 209 | 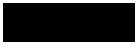 | 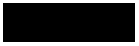 | 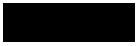 | [symbol] | [symbol] | [symbol] | 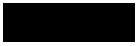 | 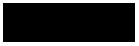 | [symbol] | [symbol] | [symbol] | [symbol] | [symbol] | [symbol] | |
| 67 | 215 | 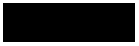 | 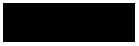 | [symbol] | [symbol] | [symbol] | [symbol] | 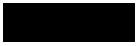 | 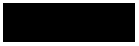 | [symbol] | [symbol] | [symbol] | [symbol] | [symbol] | [symbol] | |
| 68 | 22 | 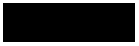 | 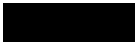 | [symbol] | [symbol] | 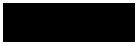 | [symbol] | 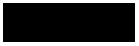 | 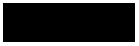 | [symbol] | [symbol] | [symbol] | [symbol] | [symbol] | [symbol] | |
| 69 | 220 | 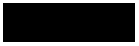 | 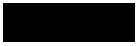 | [symbol] | [symbol] | [symbol] | [symbol] | 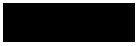 | 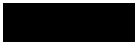 | 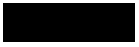 | [symbol] | [symbol] | [symbol] | [symbol] | | |
| 70 | 186 | 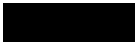 | 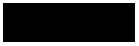 | [symbol] | [symbol] | 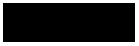 | 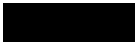 | [symbol] | [symbol] | [symbol] | [symbol] | [symbol] | [symbol] | [symbol] | [symbol] | |
| 71 | 21 | 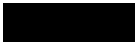 | 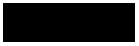 | [symbol] | [symbol] | 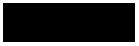 | 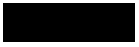 | 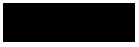 | 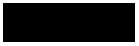 | [symbol] | [symbol] | [symbol] | [symbol] | [symbol] | [symbol] | |
| 72 | 193 | 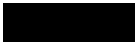 | 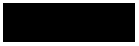 | [symbol] | [symbol] | [symbol] | [symbol] | [symbol] | [symbol] | [symbol] | [symbol] | [symbol] | [symbol] | [symbol] | [symbol] | |
| 73 | 33 | 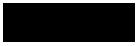 | 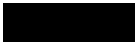 | [symbol] | [symbol] | [symbol] | [symbol] | [symbol] | [symbol] | 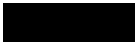 | [symbol] | [symbol] | [symbol] | [symbol] | [symbol] | |
| 74 | 34 | 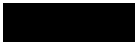 | 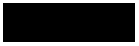 | [symbol] | [symbol] | [symbol] | [symbol] | [symbol] | [symbol] | 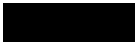 | [symbol] | [symbol] | [symbol] | [symbol] | [symbol] | |
| 75 | 227 | 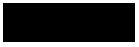 | 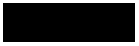 | [symbol] | [symbol] | [symbol] | [symbol] | [symbol] | [symbol] | 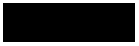 | 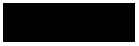 | [symbol] | [symbol] | [symbol] | [symbol] | |
| 76 | 199 | 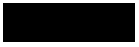 | 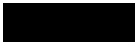 | [symbol] | [symbol] | [symbol] | [symbol] | [symbol] | [symbol] | 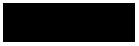 | 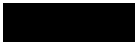 | [symbol] | [symbol] | [symbol] | [symbol] | |
| 77 | 212 | 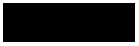 | 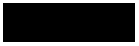 | [symbol] | [symbol] | [symbol] | [symbol] | [symbol] | [symbol] | [symbol] | 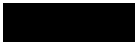 | [symbol] | [symbol] | [symbol] | [symbol] | |
| 78 | 80 | [symbol] | [symbol] | [symbol] | [symbol] | [symbol] | [symbol] | [symbol] | [symbol] | 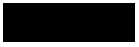 | 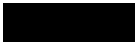 | [symbol] | [symbol] | 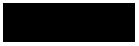 | [symbol] | |
| 79 | 16 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 80 | 79 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 81 | 74 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 82 | 75 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 83 | 100 | [symbol] | [symbol] | [symbol] | [symbol] | [symbol] | [symbol] | [symbol] | [symbol] | [symbol] | [symbol] | [symbol] | 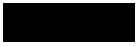 | [symbol] | [symbol] | [symbol] |
| 84 | 89 | [symbol] | [symbol] | [symbol] | [symbol] | [symbol] | [symbol] | [symbol] | [symbol] | [symbol] | [symbol] | 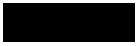 | [symbol] | [symbol] | [symbol] | |
| 85 | 102 | [symbol] | [symbol] | 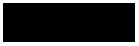 | [symbol] | [symbol] | [symbol] | [symbol] | [symbol] | [symbol] | [symbol] | [symbol] | [symbol] | [symbol] | [symbol] | |
| 86 | 86 | [symbol] | [symbol] | [symbol] | [symbol] | [symbol] | [symbol] | [symbol] | [symbol] | [symbol] | [symbol] | 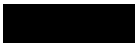 | [symbol] | [symbol] | [symbol] | |
| 87 | 103 | [symbol] | 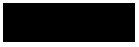 | [symbol] | [symbol] | [symbol] | [symbol] | [symbol] | [symbol] | [symbol] | [symbol] | 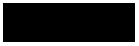 | [symbol] | [symbol] | [symbol] | |
| 88 | 93 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 89 | 108 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 90 | 146 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 91 | 192 | [symbol] | [symbol] | [symbol] | [symbol] | [symbol] | [symbol] | [symbol] | [symbol] | [symbol] | 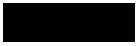 | [symbol] | [symbol] | [symbol] | [symbol] | |
| 92 | 221 | 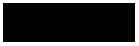 | [symbol] | [symbol] | [symbol] | [symbol] | [symbol] | [symbol] | [symbol] | [symbol] | [symbol] | [symbol] | [symbol] | [symbol] | [symbol] | |
| 93 | 234 | 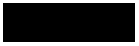 | [symbol] | [symbol] | [symbol] | [symbol] | [symbol] | [symbol] | [symbol] | [symbol] | [symbol] | [symbol] | [symbol] | [symbol] | [symbol] | |
| 94 | 231 | 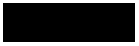 | [symbol] | [symbol] | [symbol] | [symbol] | [symbol] | [symbol] | [symbol] | [symbol] | [symbol] | [symbol] | [symbol] | [symbol] | [symbol] | |
| 95 | 224 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 96 | 35 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 97 | 259 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 98 | 251 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 99 | 1 | [symbol] | [symbol] | 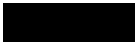 | [symbol] | [symbol] | [symbol] | [symbol] | [symbol] | [symbol] | [symbol] | [symbol] | [symbol] | [symbol] | [symbol] | |
| 100 | 265 | [symbol] | [symbol] | [symbol] | [symbol] | [symbol] | [symbol] | [symbol] | [symbol] | [symbol] | [symbol] | 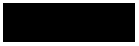 | [symbol] | [symbol] | [symbol] | |
| 101 | 5 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 102 | 368 | [symbol] | [symbol] | 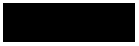 | [symbol] | [symbol] | [symbol] | [symbol] | [symbol] | [symbol] | [symbol] | [symbol] | [symbol] | [symbol] | [symbol] | |
| 103 | 304 | [symbol] | [symbol] | 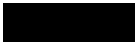 | [symbol] | [symbol] | [symbol] | [symbol] | [symbol] | [symbol] | [symbol] | 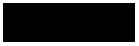 | [symbol] | [symbol] | [symbol] | |
| 104 | 398 | [symbol] | [symbol] | 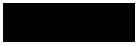 | [symbol] | 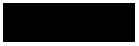 | [symbol] | [symbol] | [symbol] | 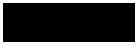 | [symbol] | [symbol] | [symbol] | [symbol] | [symbol] | |
| 105 | 302 | [symbol] | [symbol] | 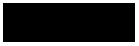 | [symbol] | [symbol] | [symbol] | [symbol] | [symbol] | [symbol] | [symbol] | 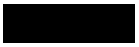 | [symbol] | [symbol] | [symbol] | |
| 106 | 429 | [symbol] | [symbol] | 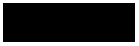 | [symbol] | [symbol] | [symbol] | [symbol] | [symbol] | [symbol] | [symbol] | [symbol] | [symbol] | [symbol] | [symbol] | |
| 107 | 402 | [symbol] | [symbol] | 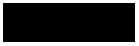 | [symbol] | [symbol] | [symbol] | [symbol] | [symbol] | [symbol] | [symbol] | [symbol] | [symbol] | [symbol] | [symbol] | |
| 108 | 322 | [symbol] | [symbol] | 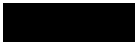 | [symbol] | [symbol] | [symbol] | [symbol] | [symbol] | [symbol] | [symbol] | 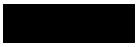 | [symbol] | [symbol] | [symbol] | |
| 109 | 90 | [symbol] | [symbol] | 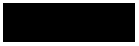 | [symbol] | [symbol] | [symbol] | [symbol] | [symbol] | [symbol] | [symbol] | 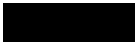 | [symbol] | [symbol] | [symbol] | |
| 110 | 105 | [symbol] | [symbol] | 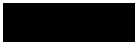 | [symbol] | [symbol] | [symbol] | [symbol] | [symbol] | [symbol] | [symbol] | 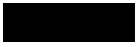 | [symbol] | [symbol] | [symbol] | |
| 111 | 37 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 112 | 272 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 113 | 273 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 114 | 275 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 115 | 274 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 116 | 41 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 117 | 285 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 118 | 288 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 119 | 282 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 120 | 45 | [symbol] | [symbol] | 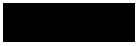 | [symbol] | [symbol] | [symbol] | [symbol] | [symbol] | [symbol] | [symbol] | [symbol] | [symbol] | [symbol] | [symbol] | |
| 121 | 11 | [symbol] | [symbol] | 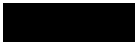 | [symbol] | [symbol] | [symbol] | [symbol] | [symbol] | [symbol] | [symbol] | [symbol] | [symbol] | [symbol] | [symbol] | |
| 122 | 54 | [symbol] | [symbol] | 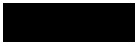 | [symbol] | [symbol] | [symbol] | [symbol] | [symbol] | [symbol] | [symbol] | 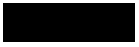 | [symbol] | [symbol] | [symbol] | |
| 123 | 52 | [symbol] | [symbol] | 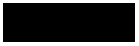 | [symbol] | [symbol] | [symbol] | [symbol] | [symbol] | [symbol] | [symbol] | 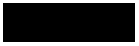 | [symbol] | [symbol] | [symbol] | |
| 124 | 51 | [symbol] | [symbol] | 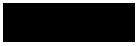 | [symbol] | [symbol] | [symbol] | [symbol] | [symbol] | [symbol] | [symbol] | 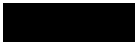 | [symbol] | [symbol] | [symbol] | |
| 125 | 14 | [symbol] | [symbol] | 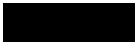 | [symbol] | 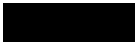 | [symbol] | [symbol] | [symbol] | [symbol] | [symbol] | [symbol] | [symbol] | [symbol] | [symbol] | |
| 126 | 66 | [symbol] | [symbol] | 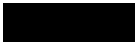 | [symbol] | [symbol] | [symbol] | [symbol] | [symbol] | [symbol] | [symbol] | [symbol] | [symbol] | [symbol] | [symbol] | |
| 127 | 4 | [symbol] | [symbol] | 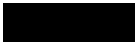 | [symbol] | [symbol] | [symbol] | [symbol] | [symbol] | [symbol] | [symbol] | [symbol] | [symbol] | [symbol] | [symbol] | |
| 128 | 7 | [symbol] | [symbol] | 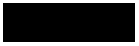 | [symbol] | [symbol] | [symbol] | [symbol] | [symbol] | [symbol] | [symbol] | [symbol] | [symbol] | [symbol] | [symbol] | |
| 129 | 68 | [symbol] | [symbol] | 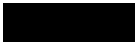 | [symbol] | [symbol] | [symbol] | [symbol] | [symbol] | [symbol] | [symbol] | [symbol] | [symbol] | [symbol] | [symbol] | |
| 130 | 67 | [symbol] | [symbol] | 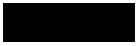 | [symbol] | [symbol] | [symbol] | [symbol] | [symbol] | [symbol] | [symbol] | [symbol] | [symbol] | [symbol] | [symbol] | |
| 131 | 15 | [symbol] | [symbol] | 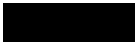 | [symbol] | [symbol] | [symbol] | [symbol] | [symbol] | [symbol] | [symbol] | 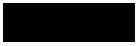 | [symbol] | [symbol] | [symbol] | |
| 132 | 335 | [symbol] | [symbol] | 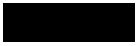 | [symbol] | [symbol] | [symbol] | [symbol] | [symbol] | [symbol] | [symbol] | [symbol] | [symbol] | [symbol] | [symbol] | |
| 133 | 354 | [symbol] | [symbol] | 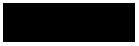 | [symbol] | [symbol] | [symbol] | [symbol] | [symbol] | [symbol] | [symbol] | [symbol] | [symbol] | [symbol] | [symbol] | |
| 134 | 355 | [symbol] | [symbol] | 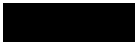 | [symbol] | [symbol] | [symbol] | [symbol] | [symbol] | [symbol] | [symbol] | 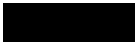 | [symbol] | [symbol] | [symbol] | |
| 135 | 365 | [symbol] | [symbol] | 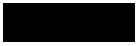 | [symbol] | [symbol] | [symbol] | [symbol] | [symbol] | [symbol] | [symbol] | [symbol] | [symbol] | [symbol] | [symbol] | |
| 136 | 372 | [symbol] | [symbol] | 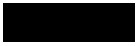 | [symbol] | [symbol] | [symbol] | [symbol] | [symbol] | [symbol] | [symbol] | [symbol] | [symbol] | [symbol] | [symbol] | |
| 137 | 376 | [symbol] | [symbol] | 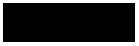 | [symbol] | [symbol] | [symbol] | [symbol] | [symbol] | [symbol] | [symbol] | [symbol] | [symbol] | [symbol] | [symbol] | |
| 138 | 389 | [symbol] | [symbol] | 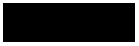 | [symbol] | [symbol] | [symbol] | [symbol] | [symbol] | [symbol] | [symbol] | [symbol] | [symbol] | [symbol] | [symbol] | |
| 139 | 391 | [symbol] | [symbol] | 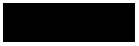 | [symbol] | [symbol] | [symbol] | [symbol] | [symbol] | [symbol] | [symbol] | [symbol] | [symbol] | [symbol] | [symbol] | |
| 140 | 399 | [symbol] | [symbol] | 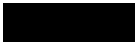 | [symbol] | [symbol] | [symbol] | [symbol] | [symbol] | [symbol] | [symbol] | [symbol] | [symbol] | [symbol] | [symbol] | |
| 141 | 405 | [symbol] | [symbol] | 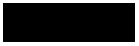 | [symbol] | [symbol] | [symbol] | [symbol] | [symbol] | [symbol] | [symbol] | [symbol] | [symbol] | [symbol] | [symbol] | |
| 142 | 407 | [symbol] | [symbol] | 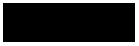 | [symbol] | [symbol] | [symbol] | [symbol] | [symbol] | [symbol] | [symbol] | [symbol] | [symbol] | [symbol] | [symbol] | |
| 143 | 418 | [symbol] | [symbol] | 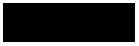 | [symbol] | [symbol] | [symbol] | [symbol] | [symbol] | [symbol] | [symbol] | [symbol] | [symbol] | [symbol] | [symbol] | |
| 144 | 420 | [symbol] | [symbol] | 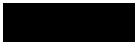 | [symbol] | [symbol] | [symbol] | [symbol] | [symbol] | [symbol] | [symbol] | [symbol] | [symbol] | [symbol] | [symbol] | |
| 145 | 422 | [symbol] | [symbol] | 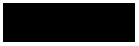 | [symbol] | [symbol] | [symbol] | [symbol] | [symbol] | [symbol] | [symbol] | [symbol] | [symbol] | [symbol] | [symbol] | |
| 146 | 374 | [symbol] | [symbol] | 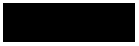 | [symbol] | [symbol] | [symbol] | [symbol] | [symbol] | [symbol] | [symbol] | [symbol] | [symbol] | [symbol] | [symbol] | |
| 147 | 303 | [symbol] | [symbol] | 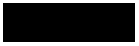 | [symbol] | [symbol] | [symbol] | [symbol] | [symbol] | [symbol] | [symbol] | [symbol] | [symbol] | [symbol] | [symbol] | |
| 148 | 388 | [symbol] | [symbol] | 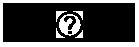 | [symbol] | [symbol] | [symbol] | [symbol] | [symbol] | [symbol] | [symbol] | 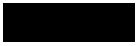 | [symbol] | [symbol] | [symbol] | |
| 149 | 403 | [symbol] | [symbol] | 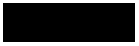 | [symbol] | [symbol] | [symbol] | [symbol] | [symbol] | [symbol] | [symbol] | [symbol] | [symbol] | [symbol] | [symbol] | |
| 150 | 390 | [symbol] | [symbol] | 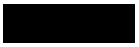 | [symbol] | [symbol] | [symbol] | [symbol] | [symbol] | [symbol] | [symbol] | [symbol] | [symbol] | [symbol] | [symbol] | |
| 151 | 380 | [symbol] | [symbol] | 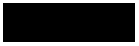 | [symbol] | [symbol] | [symbol] | [symbol] | [symbol] | [symbol] | [symbol] | [symbol] | [symbol] | [symbol] | [symbol] | |
| 152 | 424 | [symbol] | [symbol] | 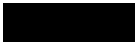 | [symbol] | [symbol] | [symbol] | [symbol] | [symbol] | [symbol] | [symbol] | 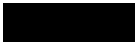 | [symbol] | [symbol] | [symbol] | |
| 153 | 340 | [symbol] | 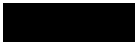 | 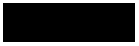 | [symbol] | 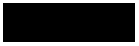 | [symbol] | [symbol] | [symbol] | [symbol] | [symbol] | [symbol] | [symbol] | [symbol] | [symbol] | |
| 154 | 415 | [symbol] | [symbol] | 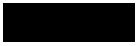 | [symbol] | [symbol] | [symbol] | [symbol] | [symbol] | [symbol] | [symbol] | [symbol] | [symbol] | [symbol] | [symbol] | |
| 155 | 412 | [symbol] | [symbol] | 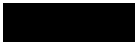 | [symbol] | [symbol] | [symbol] | [symbol] | [symbol] | [symbol] | [symbol] | [symbol] | [symbol] | [symbol] | [symbol] | |
| 156 | 382 | [symbol] | [symbol] | 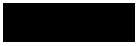 | [symbol] | [symbol] | [symbol] | [symbol] | [symbol] | [symbol] | [symbol] | [symbol] | [symbol] | [symbol] | [symbol] | |
| 157 | 314 | [symbol] | [symbol] | 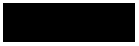 | [symbol] | [symbol] | [symbol] | [symbol] | [symbol] | [symbol] | [symbol] | [symbol] | [symbol] | [symbol] | [symbol] | |
| 158 | 370 | [symbol] | [symbol] | 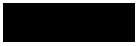 | [symbol] | 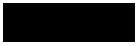 | [symbol] | [symbol] | [symbol] | [symbol] | [symbol] | 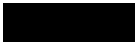 | [symbol] | [symbol] | [symbol] | |
| 159 | 409 | [symbol] | [symbol] | 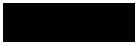 | [symbol] | [symbol] | [symbol] | [symbol] | [symbol] | [symbol] | [symbol] | [symbol] | [symbol] | [symbol] | [symbol] | |
| 160 | 432 | [symbol] | [symbol] | 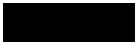 | [symbol] | [symbol] | [symbol] | [symbol] | [symbol] | [symbol] | [symbol] | [symbol] | [symbol] | [symbol] | [symbol] | |
| 161 | 312 | [symbol] | 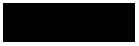 | 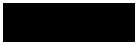 | [symbol] | 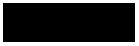 | [symbol] | [symbol] | [symbol] | [symbol] | [symbol] | [symbol] | [symbol] | [symbol] | [symbol] | |
| 162 | 331 | [symbol] | [symbol] | 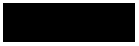 | [symbol] | [symbol] | [symbol] | [symbol] | [symbol] | [symbol] | [symbol] | [symbol] | [symbol] | [symbol] | [symbol] | |
| 163 | 334 | [symbol] | [symbol] | 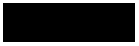 | [symbol] | 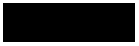 | [symbol] | [symbol] | [symbol] | [symbol] | [symbol] | [symbol] | [symbol] | [symbol] | [symbol] | |
| 164 | 386 | [symbol] | [symbol] | 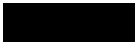 | [symbol] | [symbol] | [symbol] | [symbol] | [symbol] | [symbol] | [symbol] | [symbol] | [symbol] | [symbol] | [symbol] | |
| 165 | 419 | [symbol] | [symbol] | 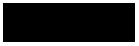 | [symbol] | 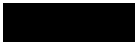 | [symbol] | [symbol] | [symbol] | [symbol] | [symbol] | [symbol] | [symbol] | [symbol] | [symbol] | |
| 166 | 319 | [symbol] | [symbol] | 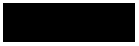 | [symbol] | [symbol] | [symbol] | [symbol] | [symbol] | [symbol] | [symbol] | 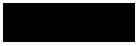 | [symbol] | [symbol] | [symbol] | |
| 167 | 342 | [symbol] | [symbol] | 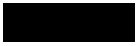 | [symbol] | [symbol] | [symbol] | [symbol] | [symbol] | [symbol] | [symbol] | [symbol] | [symbol] | [symbol] | [symbol] | |
| 168 | 313 | [symbol] | [symbol] | 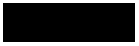 | [symbol] | 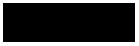 | [symbol] | [symbol] | [symbol] | [symbol] | [symbol] | [symbol] | [symbol] | [symbol] | [symbol] | |
| 169 | 406 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 170 | 426 | [symbol] | [symbol] | 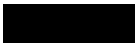 | [symbol] | [symbol] | [symbol] | [symbol] | [symbol] | [symbol] | [symbol] | [symbol] | [symbol] | [symbol] | [symbol] | |
| 171 | 379 | [symbol] | [symbol] | 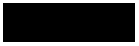 | [symbol] | 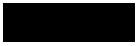 | [symbol] | [symbol] | [symbol] | [symbol] | [symbol] | [symbol] | [symbol] | [symbol] | [symbol] | |
| 172 | [symbol] | [symbol] | [symbol] | [symbol] | [symbol] | [symbol] | [symbol] | [symbol] | [symbol] | [symbol] | 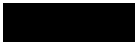 | [symbol] | [symbol] | [symbol] | | |
| 173 | 326 | 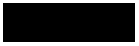 | [symbol] | 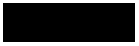 | [symbol] | [symbol] | [symbol] | 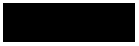 | [symbol] | [symbol] | [symbol] | [symbol] | [symbol] | [symbol] | [symbol] | |
| 174 | 346 | [symbol] | [symbol] | 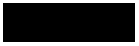 | [symbol] | 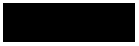 | [symbol] | [symbol] | [symbol] | [symbol] | [symbol] | [symbol] | [symbol] | [symbol] | [symbol] | |
| 175 | 361 | [symbol] | [symbol] | 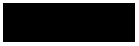 | [symbol] | [symbol] | [symbol] | [symbol] | [symbol] | [symbol] | [symbol] | 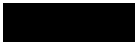 | [symbol] | [symbol] | [symbol] | |
| 176 | 332 | [symbol] | [symbol] | 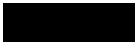 | [symbol] | [symbol] | [symbol] | [symbol] | [symbol] | [symbol] | [symbol] | [symbol] | [symbol] | [symbol] | [symbol] | |
| 177 | 417 | [symbol] | [symbol] | 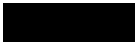 | [symbol] | [symbol] | [symbol] | [symbol] | [symbol] | [symbol] | [symbol] | [symbol] | [symbol] | [symbol] | [symbol] | |
| 178 | 362 | [symbol] | [symbol] | 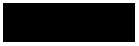 | [symbol] | [symbol] | [symbol] | [symbol] | [symbol] | [symbol] | [symbol] | 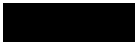 | [symbol] | [symbol] | [symbol] | |
| 179 | 400 | [symbol] | [symbol] | 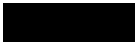 | [symbol] | [symbol] | [symbol] | [symbol] | [symbol] | [symbol] | [symbol] | 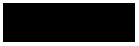 | [symbol] | [symbol] | [symbol] | |
| 180 | 433 | [symbol] | [symbol] | 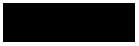 | [symbol] | [symbol] | [symbol] | [symbol] | [symbol] | [symbol] | [symbol] | [symbol] | [symbol] | [symbol] | [symbol] | |
| 181 | 301 | [symbol] | [symbol] | 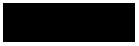 | [symbol] | 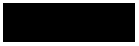 | [symbol] | [symbol] | [symbol] | [symbol] | [symbol] | 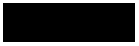 | [symbol] | 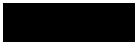 | [symbol] | |
| 182 | 315 | [symbol] | [symbol] | 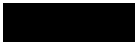 | [symbol] | 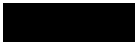 | [symbol] | [symbol] | [symbol] | [symbol] | [symbol] | 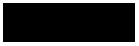 | [symbol] | [symbol] | [symbol] | |
| 183 | 296 | [symbol] | [symbol] | 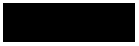 | [symbol] | [symbol] | [symbol] | [symbol] | [symbol] | [symbol] | [symbol] | [symbol] | [symbol] | [symbol] | [symbol] | |
| 184 | 367 | [symbol] | [symbol] | 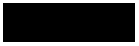 | [symbol] | [symbol] | [symbol] | [symbol] | [symbol] | [symbol] | [symbol] | [symbol] | [symbol] | [symbol] | [symbol] | |
| 185 | 328 | [symbol] | [symbol] | 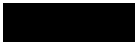 | [symbol] | [symbol] | [symbol] | [symbol] | [symbol] | [symbol] | [symbol] | [symbol] | [symbol] | [symbol] | [symbol] | |
| 186 | 351 | [symbol] | [symbol] | 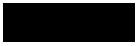 | [symbol] | [symbol] | [symbol] | [symbol] | [symbol] | [symbol] | [symbol] | [symbol] | [symbol] | [symbol] | [symbol] | |
| 187 | 371 | [symbol] | [symbol] | 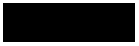 | [symbol] | [symbol] | [symbol] | [symbol] | [symbol] | [symbol] | [symbol] | 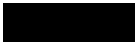 | [symbol] | [symbol] | [symbol] | |
| 188 | 394 | [symbol] | [symbol] | 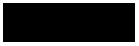 | [symbol] | [symbol] | [symbol] | [symbol] | [symbol] | [symbol] | [symbol] | 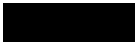 | [symbol] | [symbol] | [symbol] | |
| 189 | 404 | [symbol] | [symbol] | 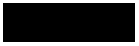 | [symbol] | [symbol] | [symbol] | [symbol] | [symbol] | [symbol] | [symbol] | 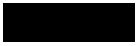 | [symbol] | [symbol] | [symbol] | |
| 190 | 423 | [symbol] | [symbol] | 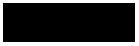 | [symbol] | [symbol] | [symbol] | [symbol] | [symbol] | [symbol] | [symbol] | [symbol] | [symbol] | [symbol] | [symbol] | |
| 191 | 435 | [symbol] | [symbol] | 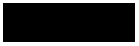 | [symbol] | [symbol] | [symbol] | [symbol] | [symbol] | [symbol] | [symbol] | [symbol] | [symbol] | [symbol] | [symbol] | |
| 192 | 410 | [symbol] | [symbol] | 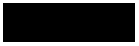 | [symbol] | 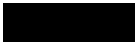 | [symbol] | [symbol] | [symbol] | [symbol] | [symbol] | [symbol] | [symbol] | [symbol] | [symbol] | |
| 193 | 318 | [symbol] | [symbol] | 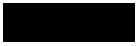 | [symbol] | [symbol] | [symbol] | [symbol] | [symbol] | [symbol] | [symbol] | [symbol] | [symbol] | [symbol] | [symbol] | |
| 194 | 425 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 195 | 427 | [symbol] | [symbol] | 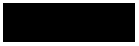 | [symbol] | [symbol] | [symbol] | [symbol] | [symbol] | [symbol] | [symbol] | [symbol] | [symbol] | [symbol] | [symbol] | |
| 196 | 369 | [symbol] | [symbol] | 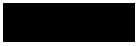 | [symbol] | [symbol] | [symbol] | [symbol] | [symbol] | [symbol] | [symbol] | [symbol] | [symbol] | [symbol] | [symbol] | |
| 197 | 393 | [symbol] | [symbol] | 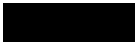 | [symbol] | [symbol] | [symbol] | [symbol] | [symbol] | [symbol] | [symbol] | [symbol] | [symbol] | [symbol] | [symbol] | |
| 198 | 358 | [symbol] | [symbol] | 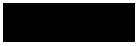 | [symbol] | [symbol] | [symbol] | [symbol] | [symbol] | [symbol] | [symbol] | [symbol] | [symbol] | [symbol] | [symbol] | |
| 199 | 421 | [symbol] | [symbol] | 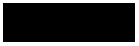 | [symbol] | [symbol] | [symbol] | [symbol] | [symbol] | [symbol] | [symbol] | [symbol] | [symbol] | [symbol] | [symbol] | |
| 200 | [symbol] | [symbol] | 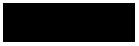 | [symbol] | [symbol] | [symbol] | [symbol] | [symbol] | [symbol] | [symbol] | 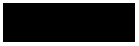 | 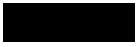 | [symbol] | [symbol] | | |
| 201 | 291 | 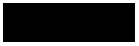 | [symbol] | 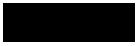 | [symbol] | [symbol] | [symbol] | 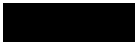 | [symbol] | [symbol] | [symbol] | [symbol] | 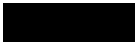 | [symbol] | [symbol] | |
| 202 | 353 | [symbol] | 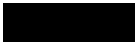 | 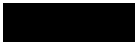 | [symbol] | 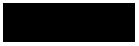 | [symbol] | [symbol] | [symbol] | [symbol] | [symbol] | 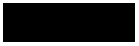 | [symbol] | [symbol] | [symbol] | |
| 203 | 295 | 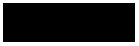 | 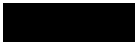 | 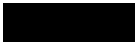 | [symbol] | 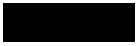 | [symbol] | 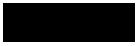 | [symbol] | [symbol] | [symbol] | [symbol] | [symbol] | [symbol] | [symbol] | |
| 204 | 194 | 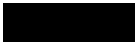 | 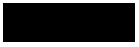 | [symbol] | [symbol] | 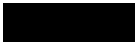 | 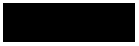 | [symbol] | [symbol] | [symbol] | [symbol] | [symbol] | [symbol] | [symbol] | [symbol] | |
| 205 | 76 | 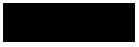 | 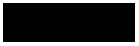 | 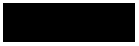 | 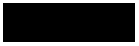 | [symbol] | [symbol] | 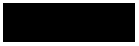 | 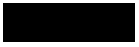 | [symbol] | [symbol] | 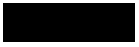 | 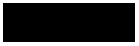 | 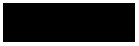 | 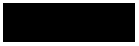 | |
| 206 | 48 | [symbol] | [symbol] | 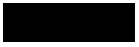 | 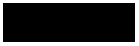 | [symbol] | [symbol] | [symbol] | [symbol] | [symbol] | [symbol] | 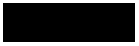 | [symbol] | [symbol] | [symbol] | |
| 207 | 87 | [symbol] | 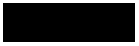 | 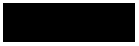 | 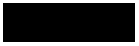 | [symbol] | [symbol] | [symbol] | [symbol] | [symbol] | [symbol] | 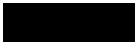 | [symbol] | [symbol] | [symbol] | |
| 208 | 92 | [symbol] | [symbol] | [symbol] | 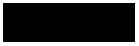 | [symbol] | [symbol] | [symbol] | [symbol] | [symbol] | [symbol] | 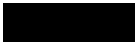 | [symbol] | [symbol] | [symbol] | |
| 209 | [symbol] | [symbol] | 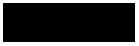 | [symbol] | [symbol] | [symbol] | [symbol] | [symbol] | [symbol] | [symbol] | [symbol] | [symbol] | [symbol] | [symbol] | | |
| 210 | [symbol] | [symbol] | 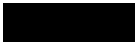 | [symbol] | 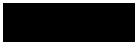 | [symbol] | [symbol] | [symbol] | [symbol] | [symbol] | 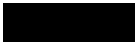 | [symbol] | [symbol] | [symbol] | | |
| 211 | [symbol] | [symbol] | 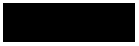 | [symbol] | [symbol] | [symbol] | [symbol] | [symbol] | [symbol] | [symbol] | [symbol] | [symbol] | [symbol] | [symbol] | | |
| 212 | 384 | [symbol] | [symbol] | 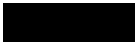 | [symbol] | [symbol] | [symbol] | [symbol] | [symbol] | [symbol] | [symbol] | [symbol] | [symbol] | [symbol] | [symbol] | |
| 213 | 408 | [symbol] | [symbol] | 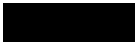 | [symbol] | [symbol] | [symbol] | [symbol] | [symbol] | [symbol] | [symbol] | [symbol] | [symbol] | [symbol] | [symbol] | |
| 214 | 396 | [symbol] | [symbol] | 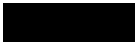 | [symbol] | [symbol] | [symbol] | [symbol] | [symbol] | [symbol] | [symbol] | 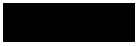 | [symbol] | [symbol] | [symbol] | |
| 215 | 179 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 216 | 255 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 217 | 81 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 218 | 82 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 219 | 83 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 220 | 85 | [symbol] | [symbol] | [symbol] | [symbol] | [symbol] | [symbol] | [symbol] | [symbol] | [symbol] | [symbol] | [symbol] | [symbol] | [symbol] | [symbol] | |
| 221 | 96 | [symbol] | [symbol] | [symbol] | [symbol] | [symbol] | [symbol] | [symbol] | [symbol] | [symbol] | [symbol] | 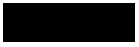 | [symbol] | [symbol] | [symbol] | |
| 222 | 97 | [symbol] | [symbol] | [symbol] | [symbol] | [symbol] | [symbol] | [symbol] | [symbol] | [symbol] | [symbol] | 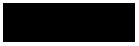 | 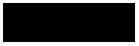 | [symbol] | [symbol] | |
| 223 | 114 | [symbol] | [symbol] | 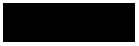 | 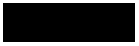 | [symbol] | [symbol] | 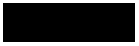 | [symbol] | [symbol] | [symbol] | [symbol] | [symbol] | [symbol] | [symbol] | |
| 224 | 158 | [symbol] | [symbol] | [symbol] | 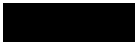 | [symbol] | [symbol] | [symbol] | [symbol] | [symbol] | [symbol] | [symbol] | [symbol] | [symbol] | [symbol] | |
| 225 | 191 | [symbol] | [symbol] | [symbol] | 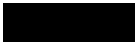 | [symbol] | [symbol] | [symbol] | [symbol] | [symbol] | [symbol] | [symbol] | [symbol] | [symbol] | [symbol] | |
| 226 | 271 | 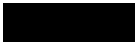 | [symbol] | [symbol] | 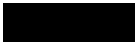 | [symbol] | [symbol] | [symbol] | [symbol] | [symbol] | [symbol] | [symbol] | [symbol] | [symbol] | [symbol] | |
| 227 | 266 | [symbol] | [symbol] | [symbol] | 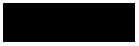 | [symbol] | [symbol] | [symbol] | [symbol] | [symbol] | [symbol] | [symbol] | [symbol] | [symbol] | [symbol] | |
| 228 | 267 | [symbol] | [symbol] | [symbol] | 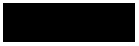 | [symbol] | [symbol] | [symbol] | [symbol] | [symbol] | [symbol] | [symbol] | [symbol] | [symbol] | [symbol] | |
| 229 | 261 | [symbol] | [symbol] | [symbol] | 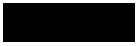 | [symbol] | [symbol] | [symbol] | [symbol] | [symbol] | [symbol] | [symbol] | [symbol] | [symbol] | [symbol] | |

| | | |
|---|---|---|
| 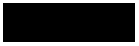 | 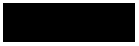 | 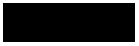 |

| | | (−) | (+) | (−) | (+) | (−) | (+) | | | | |
|---|---|---|---|---|---|---|---|---|---|---|---|
| SEQ | | [symbol] | [symbol] | [symbol] | [symbol] | [symbol] | [symbol] | | | | |
| ID | | [symbol] | [symbol] | [symbol] | [symbol] | [symbol] | [symbol] | Max | Average | Missed | |
| NO | ID: | [symbol] | [symbol] | [symbol] | [symbol] | [symbol] | [symbol] | Signal | Signal | Values | |
| 1 | 161 | [symbol] | [symbol] | [symbol] | [symbol] | [symbol] | [symbol] | [symbol] | [symbol] | [symbol] | |
| 2 | 47 | 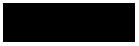 | [symbol] | [symbol] | [symbol] | [symbol] | [symbol] | [symbol] | [symbol] | [symbol] | |
| 3 | 252 | [symbol] | 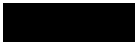 | [symbol] | [symbol] | 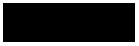 | 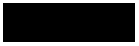 | [symbol] | [symbol] | [symbol] | |
| 4 | 98 | [symbol] | [symbol] | [symbol] | [symbol] | [symbol] | [symbol] | [symbol] | [symbol] | [symbol] | |
| 5 | 94 | [symbol] | [symbol] | [symbol] | [symbol] | [symbol] | [symbol] | [symbol] | [symbol] | [symbol] | |
| 6 | 104 | [symbol] | [symbol] | [symbol] | [symbol] | [symbol] | [symbol] | [symbol] | [symbol] | [symbol] | |
| 7 | 122 | [symbol] | [symbol] | [symbol] | [symbol] | [symbol] | [symbol] | [symbol] | [symbol] | [symbol] | |
| 8 | 115 | [symbol] | [symbol] | [symbol] | [symbol] | [symbol] | [symbol] | [symbol] | [symbol] | [symbol] | |
| 9 | 118 | [symbol] | [symbol] | [symbol] | [symbol] | [symbol] | [symbol] | [symbol] | [symbol] | [symbol] | |
| 10 | 116 | [symbol] | [symbol] | [symbol] | [symbol] | [symbol] | [symbol] | [symbol] | [symbol] | [symbol] | |
| 11 | 128 | [symbol] | [symbol] | [symbol] | [symbol] | [symbol] | [symbol] | [symbol] | [symbol] | [symbol] | |
| 12 | 189 | [symbol] | [symbol] | [symbol] | [symbol] | [symbol] | [symbol] | [symbol] | [symbol] | [symbol] | |
| 13 | 235 | [symbol] | [symbol] | [symbol] | [symbol] | [symbol] | [symbol] | [symbol] | [symbol] | [symbol] | |
| 14 | 269 | [symbol] | [symbol] | [symbol] | [symbol] | [symbol] | [symbol] | [symbol] | [symbol] | [symbol] | |
| 15 | 262 | [symbol] | [symbol] | [symbol] | [symbol] | [symbol] | [symbol] | [symbol] | [symbol] | [symbol] | |
| 16 | 263 | [symbol] | [symbol] | [symbol] | [symbol] | [symbol] | [symbol] | [symbol] | [symbol] | [symbol] | |
| 17 | 50 | [symbol] | [symbol] | [symbol] | [symbol] | [symbol] | [symbol] | [symbol] | [symbol] | [symbol] | |
| 18 | 58 | [symbol] | [symbol] | [symbol] | [symbol] | [symbol] | [symbol] | [symbol] | [symbol] | [symbol] | |
| 19 | 237 | [symbol] | [symbol] | [symbol] | [symbol] | [symbol] | [symbol] | [symbol] | [symbol] | [symbol] | |
| 20 | 333 | [symbol] | [symbol] | [symbol] | [symbol] | [symbol] | [symbol] | [symbol] | [symbol] | [symbol] | |
| 21 | 357 | [symbol] | [symbol] | [symbol] | [symbol] | [symbol] | [symbol] | [symbol] | [symbol] | [symbol] | |
| 22 | 309 | [symbol] | [symbol] | [symbol] | [symbol] | [symbol] | [symbol] | [symbol] | [symbol] | [symbol] | |
| 23 | 305 | [symbol] | [symbol] | [symbol] | [symbol] | [symbol] | [symbol] | [symbol] | [symbol] | [symbol] | |
| 24 | 155 | 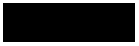 | 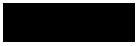 | [symbol] | [symbol] | [symbol] | [symbol] | [symbol] | [symbol] | [symbol] | |
| 25 | 292 | [symbol] | [symbol] | [symbol] | 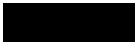 | [symbol] | [symbol] | [symbol] | [symbol] | [symbol] | |
| 26 | 120 | [symbol] | [symbol] | [symbol] | [symbol] | [symbol] | 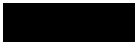 | [symbol] | [symbol] | [symbol] | |
| 27 | 202 | [symbol] | [symbol] | [symbol] | [symbol] | 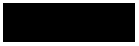 | 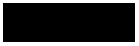 | [symbol] | [symbol] | [symbol] | |
| 28 | 196 | [symbol] | [symbol] | [symbol] | [symbol] | 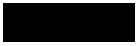 | 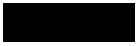 | [symbol] | [symbol] | [symbol] | |
| 29 | 253 | [symbol] | [symbol] | [symbol] | [symbol] | 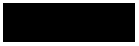 | 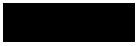 | [symbol] | [symbol] | [symbol] | |
| 30 | 250 | [symbol] | [symbol] | [symbol] | [symbol] | 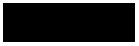 | 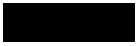 | [symbol] | [symbol] | [symbol] | |
| 31 | 152 | [symbol] | [symbol] | [symbol] | [symbol] | [symbol] | [symbol] | [symbol] | [symbol] | [symbol] | |
| 32 | 181 | [symbol] | [symbol] | 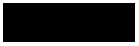 | 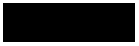 | [symbol] | [symbol] | [symbol] | [symbol] | [symbol] | |
| 33 | 107 | [symbol] | [symbol] | [symbol] | [symbol] | [symbol] | [symbol] | [symbol] | [symbol] | [symbol] | |
| 34 | 129 | [symbol] | [symbol] | [symbol] | [symbol] | [symbol] | [symbol] | [symbol] | [symbol] | [symbol] | |
| 35 | 160 | [symbol] | [symbol] | [symbol] | [symbol] | [symbol] | [symbol] | [symbol] | [symbol] | [symbol] | |
| 36 | 17 | [symbol] | [symbol] | [symbol] | [symbol] | [symbol] | [symbol] | [symbol] | [symbol] | [symbol] | |
| 37 | 19 | [symbol] | [symbol] | [symbol] | [symbol] | [symbol] | [symbol] | [symbol] | [symbol] | [symbol] | |
| 38 | 136 | [symbol] | [symbol] | [symbol] | [symbol] | [symbol] | [symbol] | [symbol] | [symbol] | [symbol] | |
| 39 | 112 | [symbol] | [symbol] | [symbol] | [symbol] | [symbol] | [symbol] | [symbol] | [symbol] | [symbol] | |
| 40 | 25 | [symbol] | [symbol] | [symbol] | [symbol] | [symbol] | [symbol] | [symbol] | [symbol] | [symbol] | |
| 41 | 26 | [symbol] | [symbol] | [symbol] | [symbol] | [symbol] | [symbol] | [symbol] | [symbol] | [symbol] | |
| 42 | 27 | [symbol] | [symbol] | [symbol] | [symbol] | [symbol] | [symbol] | [symbol] | [symbol] | [symbol] | |
| 43 | 163 | [symbol] | [symbol] | [symbol] | [symbol] | [symbol] | [symbol] | [symbol] | [symbol] | [symbol] | |
| 44 | 177 | [symbol] | [symbol] | [symbol] | [symbol] | [symbol] | [symbol] | [symbol] | [symbol] | [symbol] | |
| 45 | 167 | [symbol] | [symbol] | [symbol] | [symbol] | [symbol] | [symbol] | [symbol] | [symbol] | [symbol] | |
| 46 | 178 | [symbol] | [symbol] | [symbol] | [symbol] | [symbol] | [symbol] | [symbol] | [symbol] | [symbol] | |
| 47 | 175 | [symbol] | [symbol] | [symbol] | [symbol] | [symbol] | [symbol] | [symbol] | [symbol] | [symbol] | |
| 48 | 38 | [symbol] | [symbol] | [symbol] | [symbol] | [symbol] | [symbol] | [symbol] | [symbol] | [symbol] | |
| 49 | 60 | [symbol] | [symbol] | [symbol] | [symbol] | [symbol] | [symbol] | [symbol] | [symbol] | [symbol] | |
| 50 | 62 | [symbol] | [symbol] | [symbol] | [symbol] | [symbol] | [symbol] | [symbol] | [symbol] | [symbol] | |
| 51 | 106 | 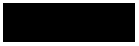 | 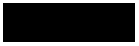 | [symbol] | [symbol] | [symbol] | [symbol] | [symbol] | [symbol] | [symbol] | |
| 52 | 170 | 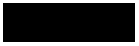 | 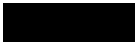 | [symbol] | [symbol] | [symbol] | [symbol] | [symbol] | [symbol] | [symbol] | |
| 53 | 183 | 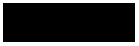 | 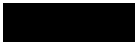 | [symbol] | [symbol] | [symbol] | [symbol] | [symbol] | [symbol] | [symbol] | |
| 54 | 29 | 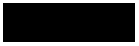 | 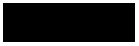 | [symbol] | [symbol] | [symbol] | [symbol] | [symbol] | [symbol] | [symbol] | |
| 55 | 187 | 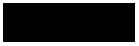 | 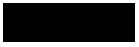 | [symbol] | [symbol] | [symbol] | [symbol] | [symbol] | [symbol] | [symbol] | |
| 56 | 219 | 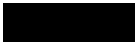 | 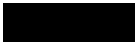 | [symbol] | [symbol] | [symbol] | [symbol] | [symbol] | [symbol] | [symbol] | |
| 57 | 222 | 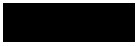 | 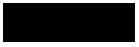 | [symbol] | [symbol] | [symbol] | [symbol] | [symbol] | [symbol] | [symbol] | |
| 58 | 30 | [symbol] | [symbol] | [symbol] | [symbol] | [symbol] | [symbol] | [symbol] | [symbol] | [symbol] | |
| 59 | 213 | [symbol] | [symbol] | [symbol] | [symbol] | [symbol] | [symbol] | [symbol] | [symbol] | [symbol] | |
| 60 | 207 | [symbol] | [symbol] | [symbol] | [symbol] | [symbol] | [symbol] | [symbol] | [symbol] | [symbol] | |
| 61 | 205 | [symbol] | [symbol] | [symbol] | [symbol] | [symbol] | [symbol] | [symbol] | [symbol] | [symbol] | |
| 62 | 237 | [symbol] | [symbol] | [symbol] | [symbol] | [symbol] | [symbol] | [symbol] | [symbol] | [symbol] | |
| 63 | 211 | [symbol] | [symbol] | [symbol] | [symbol] | [symbol] | [symbol] | [symbol] | [symbol] | [symbol] | |
| 64 | 232 | [symbol] | [symbol] | [symbol] | [symbol] | [symbol] | [symbol] | [symbol] | [symbol] | [symbol] | |
| 65 | 230 | [symbol] | [symbol] | [symbol] | [symbol] | [symbol] | [symbol] | [symbol] | [symbol] | [symbol] | |
| 66 | 209 | [symbol] | [symbol] | [symbol] | [symbol] | [symbol] | [symbol] | [symbol] | [symbol] | [symbol] | |
| 67 | 215 | [symbol] | [symbol] | [symbol] | [symbol] | [symbol] | [symbol] | [symbol] | [symbol] | [symbol] | |
| 68 | 22 | 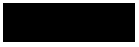 | 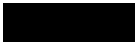 | [symbol] | [symbol] | [symbol] | [symbol] | [symbol] | [symbol] | [symbol] | |
| 69 | 220 | 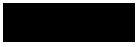 | 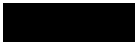 | [symbol] | [symbol] | [symbol] | [symbol] | [symbol] | [symbol] | [symbol] | |
| 70 | 186 | 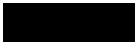 | [symbol] | [symbol] | [symbol] | [symbol] | [symbol] | [symbol] | [symbol] | [symbol] | |
| 71 | 21 | [symbol] | [symbol] | [symbol] | 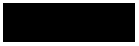 | [symbol] | [symbol] | [symbol] | [symbol] | [symbol] | |
| 72 | 193 | 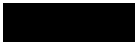 | 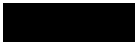 | [symbol] | [symbol] | [symbol] | [symbol] | [symbol] | [symbol] | [symbol] | |
| 73 | 33 | 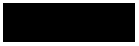 | 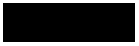 | [symbol] | [symbol] | [symbol] | [symbol] | [symbol] | [symbol] | [symbol] | |
| 74 | 34 | 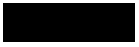 | 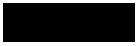 | [symbol] | [symbol] | [symbol] | [symbol] | [symbol] | [symbol] | [symbol] | |
| 75 | 227 | 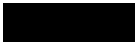 | [symbol] | [symbol] | [symbol] | [symbol] | [symbol] | [symbol] | [symbol] | [symbol] | |
| 76 | 199 | 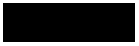 | 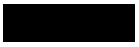 | [symbol] | [symbol] | [symbol] | [symbol] | [symbol] | [symbol] | [symbol] | |
| 77 | 212 | [symbol] | [symbol] | 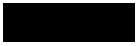 | 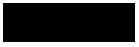 | [symbol] | [symbol] | [symbol] | [symbol] | [symbol] | |
| 78 | 80 | [symbol] | [symbol] | 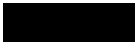 | [symbol] | [symbol] | [symbol] | [symbol] | [symbol] | [symbol] | |
| 79 | 16 | [symbol] | [symbol] | [symbol] | [symbol] | [symbol] | [symbol] | [symbol] | [symbol] | [symbol] | |
| 80 | 79 | [symbol] | [symbol] | [symbol] | [symbol] | [symbol] | [symbol] | [symbol] | [symbol] | [symbol] | |
| 81 | 74 | [symbol] | [symbol] | [symbol] | [symbol] | [symbol] | [symbol] | [symbol] | [symbol] | [symbol] | |
| 82 | 75 | [symbol] | [symbol] | [symbol] | [symbol] | [symbol] | [symbol] | [symbol] | [symbol] | [symbol] | |
| 83 | 100 | [symbol] | [symbol] | [symbol] | [symbol] | [symbol] | [symbol] | [symbol] | [symbol] | [symbol] | |
| 84 | 89 | [symbol] | [symbol] | [symbol] | [symbol] | [symbol] | [symbol] | [symbol] | [symbol] | [symbol] | |
| 85 | 102 | [symbol] | [symbol] | [symbol] | [symbol] | [symbol] | [symbol] | [symbol] | [symbol] | [symbol] | |
| 86 | 86 | [symbol] | [symbol] | [symbol] | [symbol] | [symbol] | [symbol] | [symbol] | [symbol] | [symbol] | |
| 87 | 103 | 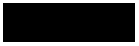 | [symbol] | [symbol] | [symbol] | [symbol] | [symbol] | [symbol] | [symbol] | [symbol] | |
| 88 | 93 | [symbol] | [symbol] | [symbol] | [symbol] | [symbol] | [symbol] | [symbol] | [symbol] | [symbol] | |
| 89 | 108 | [symbol] | [symbol] | [symbol] | [symbol] | [symbol] | [symbol] | [symbol] | [symbol] | [symbol] | |
| 90 | 146 | [symbol] | [symbol] | [symbol] | [symbol] | [symbol] | [symbol] | [symbol] | [symbol] | [symbol] | |
| 91 | 192 | 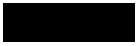 | [symbol] | [symbol] | [symbol] | 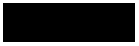 | [symbol] | [symbol] | [symbol] | [symbol] | [symbol] |
| 92 | 221 | [symbol] | [symbol] | [symbol] | [symbol] | [symbol] | [symbol] | [symbol] | [symbol] | [symbol] | |
| 93 | 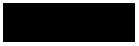 | [symbol] | [symbol] | [symbol] | 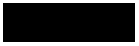 | [symbol] | [symbol] | [symbol] | [symbol] | | |
| 94 | 231 | [symbol] | [symbol] | [symbol] | [symbol] | [symbol] | [symbol] | [symbol] | [symbol] | [symbol] | |
| 95 | 224 | [symbol] | [symbol] | [symbol] | [symbol] | [symbol] | [symbol] | [symbol] | [symbol] | [symbol] | |
| 96 | 35 | [symbol] | [symbol] | [symbol] | [symbol] | [symbol] | [symbol] | [symbol] | [symbol] | [symbol] | |
| 97 | 259 | [symbol] | [symbol] | [symbol] | [symbol] | [symbol] | [symbol] | [symbol] | [symbol] | [symbol] | |
| 98 | 251 | [symbol] | [symbol] | [symbol] | [symbol] | [symbol] | [symbol] | [symbol] | [symbol] | [symbol] | |
| 99 | 1 | [symbol] | [symbol] | [symbol] | [symbol] | [symbol] | [symbol] | [symbol] | [symbol] | [symbol] | |
| 100 | 265 | [symbol] | [symbol] | [symbol] | [symbol] | [symbol] | [symbol] | [symbol] | [symbol] | [symbol] | |
| 101 | 5 | [symbol] | [symbol] | [symbol] | [symbol] | [symbol] | [symbol] | [symbol] | [symbol] | [symbol] | |
| 102 | 368 | [symbol] | [symbol] | [symbol] | [symbol] | [symbol] | [symbol] | [symbol] | [symbol] | [symbol] | |
| 103 | 304 | [symbol] | [symbol] | [symbol] | [symbol] | [symbol] | [symbol] | [symbol] | [symbol] | [symbol] | |
| 104 | 398 | [symbol] | [symbol] | [symbol] | [symbol] | [symbol] | [symbol] | [symbol] | [symbol] | [symbol] | |
| 105 | 302 | [symbol] | [symbol] | [symbol] | [symbol] | [symbol] | [symbol] | [symbol] | [symbol] | [symbol] | |
| 106 | 429 | [symbol] | [symbol] | [symbol] | [symbol] | [symbol] | [symbol] | [symbol] | [symbol] | [symbol] | |
| 107 | 402 | [symbol] | [symbol] | [symbol] | [symbol] | [symbol] | [symbol] | [symbol] | [symbol] | [symbol] | |
| 108 | 322 | [symbol] | [symbol] | [symbol] | [symbol] | [symbol] | [symbol] | [symbol] | [symbol] | [symbol] | |
| 109 | 90 | [symbol] | [symbol] | [symbol] | [symbol] | [symbol] | [symbol] | [symbol] | [symbol] | [symbol] | |
| 110 | 105 | [symbol] | [symbol] | [symbol] | [symbol] | [symbol] | [symbol] | [symbol] | [symbol] | [symbol] | |
| 111 | 37 | [symbol] | [symbol] | [symbol] | [symbol] | [symbol] | [symbol] | [symbol] | [symbol] | [symbol] | |
| 112 | 272 | [symbol] | [symbol] | [symbol] | [symbol] | [symbol] | [symbol] | [symbol] | [symbol] | [symbol] | |
| 113 | 273 | [symbol] | [symbol] | [symbol] | [symbol] | [symbol] | [symbol] | [symbol] | [symbol] | [symbol] | |
| 114 | 275 | [symbol] | [symbol] | [symbol] | [symbol] | [symbol] | [symbol] | [symbol] | [symbol] | [symbol] | |
| 115 | 274 | [symbol] | [symbol] | [symbol] | [symbol] | [symbol] | [symbol] | [symbol] | [symbol] | [symbol] | |
| 116 | 41 | [symbol] | [symbol] | [symbol] | [symbol] | [symbol] | [symbol] | [symbol] | [symbol] | [symbol] | |
| 117 | 285 | [symbol] | [symbol] | [symbol] | [symbol] | [symbol] | [symbol] | [symbol] | [symbol] | [symbol] | |
| 118 | 288 | [symbol] | [symbol] | [symbol] | [symbol] | [symbol] | [symbol] | [symbol] | [symbol] | [symbol] | |
| 119 | 282 | [symbol] | [symbol] | [symbol] | [symbol] | [symbol] | [symbol] | [symbol] | [symbol] | [symbol] | |
| 120 | 45 | [symbol] | [symbol] | [symbol] | [symbol] | [symbol] | [symbol] | [symbol] | [symbol] | [symbol] | |
| 121 | 11 | [symbol] | [symbol] | [symbol] | [symbol] | [symbol] | [symbol] | [symbol] | [symbol] | [symbol] | |
| 122 | 54 | [symbol] | [symbol] | [symbol] | [symbol] | [symbol] | [symbol] | [symbol] | [symbol] | [symbol] | |
| 123 | 52 | [symbol] | [symbol] | [symbol] | [symbol] | [symbol] | [symbol] | [symbol] | [symbol] | [symbol] | |
| 124 | 51 | 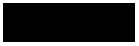 | [symbol] | [symbol] | [symbol] | [symbol] | [symbol] | [symbol] | [symbol] | [symbol] | |
| 125 | 14 | [symbol] | [symbol] | [symbol] | [symbol] | [symbol] | [symbol] | [symbol] | [symbol] | [symbol] | |
| 126 | 66 | 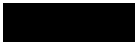 | [symbol] | [symbol] | [symbol] | [symbol] | [symbol] | [symbol] | [symbol] | [symbol] | |
| 127 | 4 | 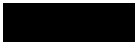 | [symbol] | [symbol] | [symbol] | [symbol] | [symbol] | [symbol] | [symbol] | [symbol] | |
| 128 | 7 | [symbol] | [symbol] | [symbol] | [symbol] | [symbol] | [symbol] | [symbol] | [symbol] | [symbol] | |
| 129 | 68 | [symbol] | [symbol] | [symbol] | [symbol] | [symbol] | [symbol] | [symbol] | [symbol] | [symbol] | |
| 130 | 67 | [symbol] | [symbol] | [symbol] | [symbol] | [symbol] | [symbol] | [symbol] | [symbol] | [symbol] | |
| 131 | 15 | [symbol] | [symbol] | [symbol] | [symbol] | [symbol] | [symbol] | [symbol] | [symbol] | [symbol] | |
| 132 | 335 | [symbol] | [symbol] | [symbol] | [symbol] | [symbol] | [symbol] | [symbol] | [symbol] | [symbol] | |
| 133 | 354 | [symbol] | [symbol] | [symbol] | [symbol] | [symbol] | [symbol] | [symbol] | [symbol] | [symbol] | |
| 134 | 355 | [symbol] | [symbol] | [symbol] | [symbol] | [symbol] | [symbol] | [symbol] | [symbol] | [symbol] | |
| 135 | 365 | [symbol] | [symbol] | [symbol] | [symbol] | [symbol] | [symbol] | [symbol] | [symbol] | [symbol] | |
| 136 | 372 | [symbol] | [symbol] | [symbol] | [symbol] | [symbol] | [symbol] | [symbol] | [symbol] | [symbol] | |
| 137 | 376 | [symbol] | [symbol] | [symbol] | [symbol] | [symbol] | [symbol] | [symbol] | [symbol] | [symbol] | |
| 138 | 389 | [symbol] | [symbol] | [symbol] | [symbol] | [symbol] | [symbol] | [symbol] | [symbol] | [symbol] | |
| 139 | 391 | [symbol] | [symbol] | [symbol] | [symbol] | [symbol] | [symbol] | [symbol] | [symbol] | [symbol] | |
| 140 | 399 | [symbol] | [symbol] | [symbol] | [symbol] | [symbol] | [symbol] | [symbol] | [symbol] | [symbol] | |
| 141 | 405 | [symbol] | [symbol] | [symbol] | [symbol] | [symbol] | [symbol] | [symbol] | [symbol] | [symbol] | |
| 142 | 407 | [symbol] | [symbol] | [symbol] | [symbol] | [symbol] | [symbol] | [symbol] | [symbol] | [symbol] | |
| 143 | 418 | [symbol] | [symbol] | [symbol] | [symbol] | [symbol] | [symbol] | [symbol] | [symbol] | [symbol] | |
| 144 | 420 | [symbol] | [symbol] | [symbol] | [symbol] | [symbol] | [symbol] | [symbol] | [symbol] | [symbol] | |
| 145 | 422 | [symbol] | [symbol] | [symbol] | [symbol] | [symbol] | [symbol] | [symbol] | [symbol] | [symbol] | |
| 146 | 374 | [symbol] | [symbol] | [symbol] | [symbol] | [symbol] | [symbol] | [symbol] | [symbol] | [symbol] | |
| 147 | 303 | [symbol] | [symbol] | [symbol] | [symbol] | [symbol] | [symbol] | [symbol] | [symbol] | [symbol] | |
| 148 | 368 | [symbol] | [symbol] | [symbol] | [symbol] | [symbol] | [symbol] | [symbol] | [symbol] | [symbol] | |
| 149 | 403 | [symbol] | [symbol] | [symbol] | [symbol] | [symbol] | [symbol] | [symbol] | [symbol] | [symbol] | |
| 150 | 390 | [symbol] | [symbol] | [symbol] | [symbol] | [symbol] | [symbol] | [symbol] | [symbol] | [symbol] | |
| 151 | 381 | [symbol] | [symbol] | [symbol] | [symbol] | [symbol] | [symbol] | [symbol] | [symbol] | [symbol] | |
| 152 | 424 | [symbol] | [symbol] | [symbol] | [symbol] | [symbol] | [symbol] | [symbol] | [symbol] | [symbol] | |
| 153 | 340 | [symbol] | [symbol] | [symbol] | [symbol] | [symbol] | [symbol] | [symbol] | [symbol] | [symbol] | |
| 154 | 415 | [symbol] | [symbol] | [symbol] | [symbol] | [symbol] | [symbol] | [symbol] | [symbol] | [symbol] | |
| 155 | 412 | [symbol] | [symbol] | [symbol] | [symbol] | [symbol] | [symbol] | [symbol] | [symbol] | [symbol] | |
| 156 | 382 | [symbol] | [symbol] | [symbol] | [symbol] | [symbol] | [symbol] | [symbol] | [symbol] | [symbol] | |
| 157 | 314 | [symbol] | [symbol] | [symbol] | [symbol] | [symbol] | [symbol] | [symbol] | [symbol] | [symbol] | |
| 158 | 370 | [symbol] | [symbol] | [symbol] | [symbol] | [symbol] | [symbol] | [symbol] | [symbol] | [symbol] | |
| 159 | 409 | [symbol] | [symbol] | [symbol] | [symbol] | [symbol] | [symbol] | [symbol] | [symbol] | [symbol] | |
| 160 | 432 | [symbol] | [symbol] | [symbol] | [symbol] | [symbol] | [symbol] | [symbol] | [symbol] | [symbol] | |
| 161 | 312 | [symbol] | [symbol] | [symbol] | [symbol] | [symbol] | [symbol] | [symbol] | [symbol] | [symbol] | |
| 162 | 331 | [symbol] | [symbol] | [symbol] | [symbol] | [symbol] | [symbol] | [symbol] | [symbol] | [symbol] | |
| 163 | 334 | [symbol] | [symbol] | [symbol] | [symbol] | [symbol] | [symbol] | [symbol] | [symbol] | [symbol] | |
| 164 | 386 | [symbol] | [symbol] | [symbol] | [symbol] | [symbol] | [symbol] | [symbol] | [symbol] | [symbol] | |
| 165 | 419 | [symbol] | [symbol] | [symbol] | [symbol] | [symbol] | [symbol] | [symbol] | [symbol] | [symbol] | |
| 166 | 319 | [symbol] | [symbol] | [symbol] | [symbol] | [symbol] | [symbol] | [symbol] | [symbol] | [symbol] | |
| 167 | 342 | [symbol] | [symbol] | [symbol] | [symbol] | [symbol] | [symbol] | [symbol] | [symbol] | [symbol] | |
| 168 | 313 | [symbol] | [symbol] | [symbol] | [symbol] | [symbol] | [symbol] | [symbol] | [symbol] | [symbol] | |
| 169 | 406 | [symbol] | [symbol] | [symbol] | [symbol] | [symbol] | [symbol] | [symbol] | [symbol] | [symbol] | |
| 170 | 426 | [symbol] | [symbol] | [symbol] | [symbol] | [symbol] | [symbol] | [symbol] | [symbol] | [symbol] | |
| 171 | 379 | [symbol] | [symbol] | [symbol] | [symbol] | [symbol] | [symbol] | [symbol] | [symbol] | [symbol] | |
| 172 | 392 | [symbol] | [symbol] | [symbol] | [symbol] | [symbol] | [symbol] | [symbol] | [symbol] | [symbol] | |
| 173 | 326 | [symbol] | [symbol] | [symbol] | [symbol] | [symbol] | [symbol] | [symbol] | [symbol] | [symbol] | |
| 174 | 346 | [symbol] | [symbol] | [symbol] | [symbol] | [symbol] | [symbol] | [symbol] | [symbol] | [symbol] | |
| 175 | 361 | [symbol] | [symbol] | [symbol] | [symbol] | [symbol] | [symbol] | [symbol] | [symbol] | [symbol] | |
| 176 | 332 | [symbol] | [symbol] | [symbol] | [symbol] | [symbol] | [symbol] | [symbol] | [symbol] | [symbol] | |
| 177 | 417 | [symbol] | [symbol] | [symbol] | [symbol] | [symbol] | [symbol] | [symbol] | [symbol] | [symbol] | |
| 178 | 362 | 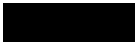 | [symbol] | [symbol] | [symbol] | [symbol] | [symbol] | [symbol] | [symbol] | [symbol] | |
| 179 | 400 | [symbol] | [symbol] | [symbol] | [symbol] | [symbol] | [symbol] | [symbol] | [symbol] | [symbol] | |
| 180 | 433 | [symbol] | [symbol] | [symbol] | [symbol] | [symbol] | [symbol] | [symbol] | [symbol] | [symbol] | |
| 181 | 301 | [symbol] | [symbol] | [symbol] | [symbol] | [symbol] | [symbol] | [symbol] | [symbol] | [symbol] | |
| 182 | 315 | [symbol] | [symbol] | [symbol] | [symbol] | [symbol] | [symbol] | [symbol] | [symbol] | [symbol] | |
| 183 | 296 | [symbol] | [symbol] | [symbol] | [symbol] | [symbol] | [symbol] | [symbol] | [symbol] | [symbol] | |
| 184 | 367 | [symbol] | [symbol] | [symbol] | [symbol] | [symbol] | [symbol] | [symbol] | [symbol] | [symbol] | |
| 185 | 328 | [symbol] | [symbol] | [symbol] | [symbol] | [symbol] | [symbol] | [symbol] | [symbol] | [symbol] | |
| 186 | 351 | [symbol] | [symbol] | [symbol] | [symbol] | [symbol] | [symbol] | [symbol] | [symbol] | [symbol] | |
| 187 | 371 | [symbol] | [symbol] | [symbol] | [symbol] | [symbol] | [symbol] | [symbol] | [symbol] | [symbol] | |
| 188 | 394 | [symbol] | [symbol] | [symbol] | [symbol] | [symbol] | [symbol] | [symbol] | [symbol] | [symbol] | |
| 189 | 404 | [symbol] | [symbol] | [symbol] | [symbol] | [symbol] | [symbol] | [symbol] | [symbol] | [symbol] | |
| 190 | 423 | [symbol] | [symbol] | [symbol] | [symbol] | [symbol] | [symbol] | [symbol] | [symbol] | [symbol] | |
| 191 | 435 | [symbol] | [symbol] | [symbol] | [symbol] | [symbol] | [symbol] | [symbol] | [symbol] | [symbol] | |
| 192 | 410 | [symbol] | [symbol] | [symbol] | [symbol] | [symbol] | [symbol] | [symbol] | [symbol] | [symbol] | |
| 193 | 218 | 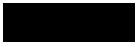 | [symbol] | [symbol] | [symbol] | [symbol] | [symbol] | [symbol] | [symbol] | [symbol] | |
| 194 | 425 | 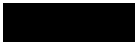 | [symbol] | [symbol] | [symbol] | [symbol] | [symbol] | [symbol] | [symbol] | [symbol] | |
| 195 | 427 | [symbol] | [symbol] | [symbol] | [symbol] | [symbol] | [symbol] | [symbol] | [symbol] | [symbol] | |
| 196 | 369 | [symbol] | [symbol] | [symbol] | [symbol] | [symbol] | [symbol] | [symbol] | [symbol] | [symbol] | |
| 197 | 393 | 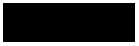 | [symbol] | [symbol] | [symbol] | [symbol] | [symbol] | [symbol] | [symbol] | [symbol] | |
| 198 | 358 | 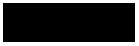 | [symbol] | [symbol] | [symbol] | [symbol] | [symbol] | [symbol] | [symbol] | [symbol] | |
| 199 | 421 | [symbol] | [symbol] | [symbol] | [symbol] | [symbol] | [symbol] | [symbol] | [symbol] | [symbol] | |
| 200 | 436 | [symbol] | [symbol] | [symbol] | [symbol] | [symbol] | [symbol] | [symbol] | [symbol] | [symbol] | |
| 201 | 291 | [symbol] | [symbol] | [symbol] | [symbol] | [symbol] | [symbol] | [symbol] | [symbol] | [symbol] | |
| 202 | 353 | [symbol] | [symbol] | [symbol] | [symbol] | [symbol] | [symbol] | [symbol] | [symbol] | [symbol] | |
| 203 | 295 | [symbol] | [symbol] | [symbol] | [symbol] | [symbol] | [symbol] | [symbol] | [symbol] | [symbol] | |
| 204 | 194 | [symbol] | [symbol] | [symbol] | [symbol] | [symbol] | [symbol] | [symbol] | [symbol] | [symbol] | |
| 205 | 76 | 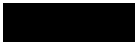 | [symbol] | [symbol] | [symbol] | [symbol] | [symbol] | [symbol] | [symbol] | [symbol] | |
| 206 | 48 | [symbol] | [symbol] | [symbol] | [symbol] | [symbol] | [symbol] | [symbol] | [symbol] | [symbol] | |
| 207 | 87 | [symbol] | [symbol] | [symbol] | [symbol] | [symbol] | [symbol] | [symbol] | [symbol] | [symbol] | |
| 208 | 92 | [symbol] | [symbol] | [symbol] | 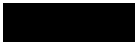 | [symbol] | [symbol] | [symbol] | [symbol] | [symbol] | |
| 209 | 336 | [symbol] | [symbol] | [symbol] | [symbol] | [symbol] | [symbol] | [symbol] | [symbol] | [symbol] | |
| 210 | 442 | [symbol] | [symbol] | [symbol] | [symbol] | [symbol] | [symbol] | [symbol] | [symbol] | [symbol] | |
| 211 | 356 | [symbol] | [symbol] | [symbol] | [symbol] | [symbol] | [symbol] | [symbol] | [symbol] | [symbol] | |
| 212 | 384 | [symbol] | [symbol] | [symbol] | [symbol] | [symbol] | [symbol] | [symbol] | [symbol] | [symbol] | |
| 213 | 408 | [symbol] | [symbol] | [symbol] | [symbol] | [symbol] | [symbol] | [symbol] | [symbol] | [symbol] | |
| 214 | 396 | [symbol] | [symbol] | [symbol] | [symbol] | [symbol] | [symbol] | [symbol] | [symbol] | [symbol] | |
| 215 | 179 | [symbol] | [symbol] | 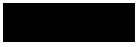 | 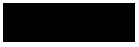 | [symbol] | [symbol] | [symbol] | [symbol] | [symbol] | |
| 216 | 255 | [symbol] | [symbol] | 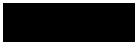 | 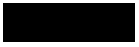 | [symbol] | [symbol] | [symbol] | [symbol] | [symbol] | |
| 217 | 81 | [symbol] | [symbol] | [symbol] | [symbol] | [symbol] | [symbol] | [symbol] | [symbol] | [symbol] | |
| 218 | 82 | [symbol] | [symbol] | [symbol] | [symbol] | [symbol] | [symbol] | [symbol] | [symbol] | [symbol] | |
| 219 | 83 | [symbol] | [symbol] | [symbol] | [symbol] | [symbol] | [symbol] | [symbol] | [symbol] | [symbol] | |
| 220 | 85 | [symbol] | [symbol] | [symbol] | [symbol] | [symbol] | [symbol] | [symbol] | [symbol] | [symbol] | |
| 221 | 96 | [symbol] | [symbol] | [symbol] | [symbol] | [symbol] | [symbol] | [symbol] | [symbol] | [symbol] | |
| 222 | 97 | [symbol] | [symbol] | [symbol] | [symbol] | [symbol] | [symbol] | [symbol] | [symbol] | [symbol] | |
| 223 | 114 | [symbol] | [symbol] | [symbol] | [symbol] | [symbol] | [symbol] | [symbol] | [symbol] | [symbol] | |
| 224 | 158 | [symbol] | [symbol] | [symbol] | [symbol] | [symbol] | [symbol] | [symbol] | [symbol] | [symbol] | |
| 225 | 191 | [symbol] | [symbol] | [symbol] | [symbol] | [symbol] | [symbol] | [symbol] | [symbol] | [symbol] | |
| 226 | 271 | [symbol] | [symbol] | [symbol] | [symbol] | [symbol] | [symbol] | [symbol] | [symbol] | [symbol] | |
| 227 | 266 | [symbol] | [symbol] | [symbol] | [symbol] | [symbol] | [symbol] | [symbol] | [symbol] | [symbol] | |
| 228 | 267 | [symbol] | [symbol] | [symbol] | [symbol] | [symbol] | [symbol] | [symbol] | [symbol] | [symbol] | |
| 229 | 261 | [symbol] | [symbol] | [symbol] | [symbol] | [symbol] | 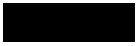 | [symbol] | [symbol] | [symbol] | |

| SEQ | | | | | | | |
|---|---|---|---|---|---|---|---|
| ID | | corresponding human or mouse | | | | | |
| NO | # | mouse EST description | consensus sequence (see FIG. 5) | PL−4BBDEnor | PL−2BBDEnor | PL+BBDEnor | |
| | | 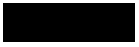 | | sple | panc | liv | |
| 290 | 1 | AA107035_guanylate_cyclase_activat | orth Homo sapiens guanylate cyclase | [symbol] | [symbol] | [symbol] | |
| | | or_2b_(retina) | activator 2B (uroguanylin) | | | | |
| | | | (GUCA2B), mRNA cr: gi- | | | | |
| | | | 6005773/// | | | | |
| | | | [Human_jongleur_201102.1395.C1] | | | | |
| | | | ; h_jongleur(strong | | | | |
| | | | homology):gi_1657021_gb_AA1070 | | | | |
| | | | 35.1_AA107035 | | | | |
| 97 | 2 | AA671177_RIKEN_cDNA_5930412 | see Table 5 | [symbol] | [symbol] | [symbol] | |
| | | E23_gene | | | | | |
| 291 | 3 | W17866_uterine-specific_proline- | Mus musculus uterine-specific | [symbol] | [symbol] | [symbol] | |
| | | rich_acidic_protein | proline-rich acidic protein (Upa), | | | | |
| | | | mRNA cr: gi-6678510/// | | | | |
| | | | [Mouse_jongleur_201102.4046.C1] | | | | |
| | | | m_jongluer(identity):gi_1292452_gb | | | | |
| | | | _W17866.1_W17866 | | | | |
| 292 | 4 | AA242201_serine_protease_inhibitor, _Kazal_type_3 | Homo sapiens serine protease inhibitor, Kazal type 1 (SPINK1), mRNA cr: gi-4507178/// [Human_jongluer_201102.8585.C1] | [symbol] | 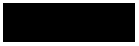 | [symbol] | |
| 293 | 5 | AA242102_zillin | Homo sapiens villin (VIL1), | [symbol] | [symbol] | [symbol] | |
| | | | mRNA cr: gi-6005943/// | | | | |
| | | | [Human_jongluer_201102.1802.C1] | | | | |
| 294 | 6 | A1046562_lactate_dehydrogenase_3,_{—} | orth 7p07c08.x1 Homo sapiens | [symbol] | [symbol] | [symbol] | |
| | | C_chain,_sperm_specific | cDNA 3′ end as: gi-11448929/// | | | | |
| | | | /clone=IMAGE:3644943 | | | | |
| | | | /clone_end=3′ /gb=BF436690 | | | | |
| | | | /gi=11448929 /ug=Hs.319750 | | | | |
| | | | /len=548. Weakly similar to | | | | |
| | | | DEHULC L-lactate dehydrogenase | | | | |
| | | | (EC 1.1.1.27)chain X-human | | | | |
| | | | [H.sapiens] [gnl\|UG\|Hs#S2910008]; | | | | |
| | | | h_jongleur(identity):gi_3294849_gb | | | | |
| | | | _A1046562.1_A1046562 | | | | |
| | | 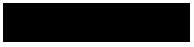 | | spl | panc | liv | |
| 127 | 1 | A1481982_EST | see Table 5 | [symbol] | 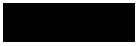 | [symbol] | |
| 295 | 2 | A1549624_RIKEN_cDNA_0610041E 09_gene | H4009E10-5 NIA Mouse 7.4K cDNA Clone Set Mus musculus cDNA clone H4009E10 5′; est(identity):gi_4481987_gb_A1549 624.1_A1549624 | 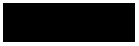 | 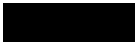 | [symbol] | |
| | | 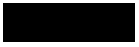 | | spl | pan | liv | |
| 91 | 1 | AA762401_ESTs | see Table 5 | [symbol] | [symbol] | [symbol] | |
| 296 | 2 | A1481911_Mus_Musculus,_clone_M | Homo sapiens cell-type T-cell | [symbol] | [symbol] | [symbol] | |
| | | GC6727,_mRNA,_complete_cds | immunoglobulin gamma chain, V | | | | |
| | | | region (IGHV@) mRNA, partial cds | | | | |
| | | | cr: gi-185240/// /cds=(0,393) | | | | |
| | | | /gb=L03177 /gi=185420 | | | | |
| | | | /ug=Hs.402428 /len=394 | | | | |
| | | | [Human_jongluer_201102.56.CB10 | | | | |
| | | | 0] | | | | |
| 55 | 1 | AA760002_beta- | see Table 5 | [symbol] | [symbol] | [symbol] | |
| | | glucuronidase_structural | | | | | |
| | | 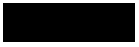 | | spl | panc | liv | |
| 96 | 1 | W34620_kallikrein_26 | see Table 5 | [symbol] | [symbol] | [symbol] | |
| 111 | 2 | AA498574_kallikrein_6 | see Table 5 | 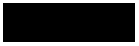 | [symbol] | [symbol] | |
| 82 | 3 | AA760185_carbonic_anhydrase_6 | see Table 5 | [symbol] | [symbol] | [symbol] | |
| 80 | 4 | W36921_demilune_cell_and_parotid_{—} | see Table 5 | [symbol] | [symbol] | [symbol] | |
| | | protein | | | | | |
| 79 | 5 | A1326499_epidermal_growth_factor | see Table 5 | [symbol] | [symbol] | [symbol] | |
| 297 | 6 | AA466852_cyesteine_rich_protein_61 | orth Homo sapiens cysteine-rich | [symbol] | [symbol] | [symbol] | |
| | | | angiogenic inducer, 61 (CYR61), | | | | |
| | | | mRNA cr: gi-4504612/// | | | | |
| | | | [Human_jongleur_201102.8413.C2] | | | | |
| | | | ; | | | | |
| | | | h_jongleur(identity):gi_2192992_gb | | | | |
| | | | _AA466852.1_AA466852 | | | | |
| 298 | 7 | AA612413_sitinase,_acidic | Homo sapiens eosinophil chemolactic cytokine (TSA1902), mRNA cr: gi-11141866/// [Human_jongluer_201102.2578.C2] | [symbol] | [symbol] | 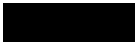 | |
| | | 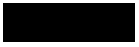 | | spl | panc | liv | |
| 299 | 1 | AA445731_heat_shock_protein_cogn ate_70,_testis | orth Homo sapiens heat shock 70kDa protein IB (HSAP1B), mRNA cr: gi-5579470/// [Human_jongluer_201102.8523.C4]; h_jongluer(identity):gi_2158414_gb _AA445731.1_AA445731 | 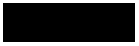 | [symbol] | [symbol] | |
| 300 | 2 | AA145895_heat_shock_protein,_70_k | Homo sapiens heat shock 70kDa | [symbol] | [symbol] | [symbol] | |
| | | Da_1 | protein 1A (HSPA1A), mRNA cr: gi- | | | | |
| | | | 557469/// | | | | |
| | | | [Human_jongluer_201102.8523.C2] | | | | |
| 246 | 3 | AA067863_RIKEN_cDNA_23100573 18_gene | see Table 5 | [symbol] | [symbol] | 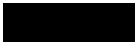 | |
| 96 | 4 | W34620_kallikrein_26 | see Table 5 | [symbol] | [symbol] | [symbol] | |
| 111 | 5 | AA498574_kallikrein_6 | see Table 5 | 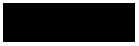 | [symbol] | [symbol] | |
| 301 | 6 | AA002910_FBJ_osteosarcoma_oncog | orth Homo sapiens cDNA FLJ40060 | [symbol] | [symbol] | [symbol] | |
| | | ene | fis, clone TCOLN2000236, highly | | | | |
| | | | similar to P55-C-FOS PROTO- | | | | |
| | | | ONCOGENE PROTEIN si: gi- | | | | |
| | | | 21757114/// | | | | |
| | | | [Human_jongluer_201102.c1.192.si | | | | |
| | | | nglet]; | | | | |
| | | | h_jongluer(identity):gi_1446351_gb | | | | |
| | | | _AA002910.1_AA002910 | | | | |
| 302 | 7 | AA433639_heat_shock_protein_cogn ate_70,_testis | Homo sapiens heat shock 70kDa protein 1A (HSPA1A), mRNA cr: gi- 5579469/// [Human_jongluer_201102.8523.C2] | 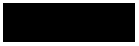 | [symbol] | [symbol] | |
| | | 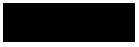 | | spl | panc | liv | |
| 218 | 1 | AA066763_hemoglobin,_beta_adult_{—} | see Table 5 | [symbol] | [symbol] | [symbol] | |
| | | major_chain | | | | | |
| 219 | 2 | W15809_hemoglobin,_beta_adult_ma | see Table 5 | [symbol] | [symbol] | [symbol] | |
| | | jor_chain | | | | | |
| 303 | 3 | W14593_kinesin- | orth Homo sapiens kinesin- | [symbol] | [symbol] | [symbol] | |
| | | associated_protein_3 | associated protein 3 (KIFAP3), | | | | |
| | | | mRNA cr: gi-18105053/// | | | | |
| | | | [Human_jongluer_201102.11853.C1 | | | | |
| | | | ]; | | | | |
| | | | h_jongluer(identity):gi_1537349_gb | | | | |
| | | | _W14953.1_W14953 | | | | |
| 217 | 4 | AA106071_hemoglobin,_beta_adult_{—} | see Table 5 | [symbol] | [symbol] | [symbol] | |
| | | major_chain | | | | | |
| 250 | 5 | A1614738_RIKEN_cDNA_261030111 5_gene | see Table 5 | [symbol] | 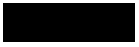 | [symbol] | |
| 249 | 6 | AA1447967_ESTs | see Table 5 | [symbol] | 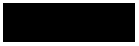 | [symbol] | |
| 251 | 7 | A1551391_ESTs,_Weakly_similar_to_{—FVI}_MOUSE_FRIEND_VIRUS_SUS CEPTIBILITY_PROTEIN_1_[M.mus culus] | see Table 5 | [symbol] | 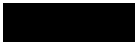 | [symbol] | |
| 222 | 8 | AA289586_protein_kinase_C,_beta | see Table 5 | [symbol] | [symbol] | [symbol] | |
| | | 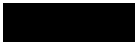 | | spl | panc | liv | |
| 304 | 1 | AA656694_cytochrome_P450,_2b9,_{—phenobarbitol}_inducible,_type_a | Homo sapiens cytochrome P450, subfamily IIB (phenobarbital- inducible), polypeptide 6 (CYP2B6), mRNA cr: gi-2052261/// [Human_jongluer_201102.5324.C1] | [symbol] | [symbol] | 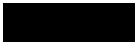 | |
| 18 | 2 | AA231099_necdin | see Table 5 | [symbol] | [symbol] | [symbol] | |
| 305 | 3 | AA510906_Mus_musculus_Cyp4v3_{—} | Homo sapiens similar to data | [symbol] | [symbol] | [symbol] | |
| | | mRNA_for_family_4_cytochrome_P4 | source:MGD, source | | | | |
| | | 50,_complete_cds | key:MGI:102849, | | | | |
| | | | evidence:ISS-kallikrein B, plasma | | | | |
| | | | 1-putative (LOC201859), mRNA as: | | | | |
| | | | gi-20472737/// | | | | |
| | | | [gi\|20472737\|rel\|XM_114391.1\|] | | | | |
| 306 | 4 | AA606610_RIKEN_cDNA_2510002 | HUMANADPDLB Human mRNA for | [symbol] | [symbol] | [symbol] | |
| | | C21_gene | NADP dependent leukotriene b4 12- | | | | |
| | | | hydroxydehydrogenase, partial cds | | | | |
| 290 | 5 | AA107035_guanylate_cyclase_activat | see Table 5 | [symbol] | [symbol] | [symbol] | |
| | | or_2b_(retina) | | | | | |
| 7 | 6 | A1323053_forkhead_box_D3 | see Table 5 | [symbol] | [symbol] | [symbol] | |
| 308 | 7 | AA571365_Mus_musculus_MRPS6_{—} | Homo sapiens mitochondrial | [symbol] | [symbol] | [symbol] | |
| | | mRNA_for_mitochondrial_ribosomal_{—} | nuclear gene encoding mitochondrial | | | | |
| | | | protein, mRNA cr: gi-16554615/// | | | | |
| | | | [Human_jongluer_201102.8885.C2] | | | | |
| 309 | 4 | AA822473_DNA_segment,_Chr_8,_B | Homo sapiens D-lactate | [symbol] | [symbol] | [symbol] | |
| | | righam_&_Women's_Genetics_1320_{—} | dehydrogenase (LDHD), mRNA cr: | | | | |
| | | expressed | gi-23821028/// | | | | |
| | | | [Human_jongluer_201102.8179.C1] | | | | |
| 235 | 9 | AA062408_ESTs,_Moderately_simila r_to_proteasome_inhibitor_hP131_sub unit_[H.sapiens] | see Table 5 | [symbol] | [symbol] | 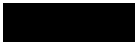 | |
| 239 | 10 | AA871395_Mus_musculus,_Similar_1 | see Table 5 | [symbol] | [symbol] | [symbol] | |
| | | o_ectonucleotide_pyrophosphatase/ph | | | | | |
| | | osphodistearate_3,_clone_IMAGE:34 | | | | | |
| | | 95326,_mRNA,_partial_cds | | | | | |
| 10 | 11 | AA518917_slug,_chicken_homolog | see Table 5 | [symbol] | [symbol] | [symbol] | |
| 310 | 12 | AA619789_methylenetetrahydrofolate | Homo sapiens methylene | [symbol] | [symbol] | [symbol] | |
| | | _dehydrogenase_(NAD+_dependent), | (NAD+ dependent), | | | | |
| | | _methyltetrahydrofolate_cyclohydro | methyltetrahydrolate | | | | |
| | | lase | cyclohydrolase (MTHFD2), nuclear | | | | |
| | | | gene encoding mitochondrial | | | | |
| | | | protein, mRNA cr: gi-13699869/// | | | | |
| | | | [Human_jongluer_201102.3990.C6] | | | | |
| 36 | 13 | AA529824_fatty_acid_binding_protei | see Table 5 | [symbol] | [symbol] | [symbol] | |
| | | n_2,_intestinal | | | | | |
| 34 | 14 | A1550591_RIKEN_cDNA_2010003F | see Table 5 | [symbol] | [symbol] | [symbol] | |
| | | 10_gene | | | | | |
| 37 | 15 | AA869173_defensin_related_cryptdin | see Table 5 | [symbol] | [symbol] | [symbol] | |
| | | _6 | | | | | |
| 39 | 16 | AA623172_defensin_related_cryptdin | see Table 5 | [symbol] | [symbol] | [symbol] | |
| | | _6 | | | | | |
| 311 | 17 | A1893944_forkhead_box_G1 | orth Homo sapiens. Similar to Forkhead-like transcription factor BF 1, clone MGC:33583 IMAGE:4823883, mRNA, complete cds gi:23241914/// [Human_jongluer_201102.c1.11482 singlet]; h_jongluer(identity):gi_5599846_gb _A1893944.1_A1893944 | [symbol] | [symbol] | 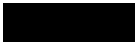 | |
| 236 | 18 | AA619953_ESTs,_Weakly_similar_to | see Table 5 | [symbol] | [symbol] | [symbol] | |
| | | _T25027_hypothetical_protein_T20D | | | | | |
| | | 3.5_- | | | | | |
| | | _Caenorhabditis_elegans_[C.elegans] | | | | | |
| 8 | 19 | AA034678_rhotekin | see | Table 5 | [symbol] | [symbol] | [symbol] |
| 90 | 20 | AA727521_Mus_Musculus_11_kDa_s | see Table 5 | [symbol] | [symbol] | [symbol] | |
| | | ecreted_protein_precursor,_mRNA,_c | | | | | |
| | | omplete_cds | | | | | |
| 71 | 21 | AA822098_apolipoprotein_A-1 | see Table 5 | [symbol] | [symbol] | [symbol] | |
| | | 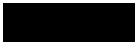 | | spl | panc | liv | |
| 48 | 1 | AA619407_pancreatitis- associated_protein | see Table 5 | 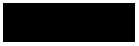 | 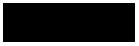 | [symbol] | |
| 312 | 2 | AA237986_cytochrome_P450,_3a25 | Homo sapiens cytochrome P450 | [symbol] | [symbol] | [symbol] | |
| | | | variant 3A7 (CYP3A7) mRNA, | | | | |
| | | | complete cds: gi-12082808/// | | | | |
| | | | [Human_jongluer_201102.c1.90.sing | | | | |
| | | | let] | | | | |
| 31 | 3 | W56983_peripheral_myelin_protein,_{—} | see Table 5 | [symbol] | [symbol] | [symbol] | |
| | | 22_kDa | | | | | |
| 290 | 4 | AA107035_guanylate_cyclase_activat | see Table 5 | [symbol] | [symbol] | [symbol] | |
| | | or_2b_(retina) | | | | | |
| 53 | 5 | AA110886_amnionless | see Table 5 | [symbol] | [symbol] | [symbol] | |
| 314 | 6 | AA109873_RIKEN_cDNA_0610010 | Homo sapiens hypothetical protein | [symbol] | [symbol] | [symbol] | |
| | | E05_gene | FLJ10830 (FLJ10830), mRNA cr: gi- | | | | |
| | | | 8922698/// | | | | |
| | | | [Human_jongluer_201102.3671.C2] | | | | |
| 315 | 2 | A1323162_dipeptidase_1_(renal) | Homo sapiens dipeptidase 1 (renal) | [symbol] | [symbol] | [symbol] | |
| | | | (DPEP1), mRNA cr: gi-4758189/// | | | | |
| | | | [Human_jongluer_201102.8623.C1] | | | | |
| 41 | 8 | AA871838_phospholipase_A2,_group | see Table 5 | [symbol] | [symbol] | [symbol] | |
| | | _IIA_(platelets,_synovial_fluid) | | | | | |
| 24 | 9 | AA656394_RIKEN_cDNA_111025H 15_gene | see Table 5 | 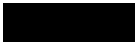 | [symbol] | [symbol] | |
| 304 | 10 | AA656694_cytochrome_P450,_2b9,_pheobarbital_inducible,_type_a | see above | [symbol] | [symbol] | 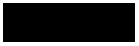 | |
| 46 | 11 | W15890_guanylate_cyclase_activator | see Table 5 | [symbol] | [symbol] | [symbol] | |
| | | _2_(guanylin_2,_intestinal,_heatstable | | | | | |
| | | ) | | | | | |
| 317 | 12 | A1325330_cytochrome_P450,_2b13,_{—phenobarbitol}_inducible,_type_c | Homo sapiens cytochrome P450, subfamily HB (phenobarbital- inducible), polypeptide 6 (CYP2B6), mRNA cr: gi-2052226/// [Human_jongleur_201102.5324.C1] | [symbol] | [symbol] | 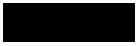 | |
| 292 | 13 | AA242201_serine_protease_inhibitor, _Kazal_type_3 | see Table 5 | [symbol] | 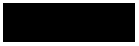 | [symbol] | |
| 1 | 14 | AA516927_sphingosine_phosphate_ly ase_1 | see Table 5 | [symbol] | [symbol] | [symbol] | |
| 319 | 15 | AA638765_metallobioncin_1 | Homo sapiens metallothionein 2 pseudogene 1 (processed) (MT2P1) on chromosome 4 | [symbol] | [symbol] | 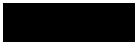 | |
| 71 | 16 | AA822098_apolipoprotein_A-1 | see Table 5 | [symbol] | [symbol] | [symbol] | |
| | | 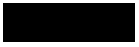 | | spl | panc | liv | |
| 295 | 1 | A1549624_RIKEN_cDNA_0610041E 09_gene | see above | 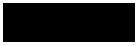 | 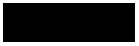 | [symbol] | |
| 130 | 2 | AA617112_ESTs | see Table 5 | [symbol] | [symbol] | [symbol] | |
| 123 | 3 | A1604587_ESTs | see Table 5 | [symbol] | [symbol] | [symbol] | |
| 321 | 4 | AA544433_dihydroorotate_dehydroge | Homo sapiens dihydroorotate | [symbol] | [symbol] | [symbol] | |
| | | nase | dehydrogenase (DHODH), nuclear | | | | |
| | | | gene encoding mitochondrial | | | | |
| | | | protein, mRNA cr: gi-16753322/// | | | | |
| | | | [Human_jongleur_201102.3057.C1] | | | | |
| 99 | 5 | AA469630_choline_kinase | see Table 5 | 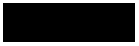 | 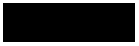 | [symbol] | |
| 127 | 6 | A1481982_ESTs | see Table 5 | [symbol] | 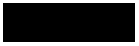 | [symbol] | |
| 286 | 7 | A1552902_RIKEN_cDNA_170004910 | see Table 5 | [symbol] | [symbol] | [symbol] | |
| | | 1_gene | | | | | |
| 195 | 8 | AA607267_Public_domain_EST | see Table 5 | [symbol] | 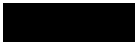 | [symbol] | |
| 255 | 9 | A1549654_programmed_cell_death_1 0 | see Table 5 | [symbol] | 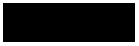 | [symbol] | |
| 322 | 10 | AA561920_Ra1BPi_associated_Eps_domain_containing_protein | Homo sapiens RALBP1 associated Eps domain containing 1 (REPS1), mRNA cr: gi-13994295/// [Human_jongleur_201102.9400.C4] | [symbol] | 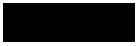 | [symbol] | |
| 170 | 11 | A1036904_ESTs,_Weakly_similar_to_X-LINKED_LYMPHOCYTE- REGULATED_PROTEIN_PMI_[M. musculus] | see Table 5 | [symbol] | [symbol] | [symbol] | |
| 251 | 12 | A1551391_ESTs,_Weakly_similar_to_FVI_MOUSE_FRIEND_VIRUS_SUS CEPTIBILITY_PROTEIN_1_(M.mus culus] | see Table 5 | [symbol] | 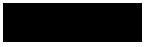 | [symbol] | |
| 338 | 13 | AA265406_nuclear_factor_I/B | Homo sapiens nuclear factor I/B | [symbol] | [symbol] | [symbol] | |
| | | | (MF1B), mRNA | | | | |
| 214 | 14 | A1587825_RIKEN_cDNA_0710001D | see Table 5 | [symbol] | [symbol] | [symbol] | |
| | | 07_gene | | | | | |
| 324 | 15 | AA146478_M.musculus_mRNA_(3C | Mus musculus, clone MGC:6342 | [symbol] | [symbol] | [symbol] | |
| | | 10)_for_IgA_V-D-J-heavy_chain | IMAGE:3488305, mRNA, complete | | | | |
| | | | cds: cr: gi-12805256/// | | | | |
| | | | [Mouse_jongleur_201102.2422.CB1 | | | | |
| | | | 8]; | | | | |
| | | | m_jongleur(identity):gi_1715876_gb | | | | |
| | | | _AA146478.1_AA146478 | | | | |
| 325 | 16 | A1158775_protein_tyrosine_phosphat | orth Homo sapiens protein tyrosine | [symbol] | [symbol] | [symbol] | |
| | | ase,_receptor-type,_M | phosphatase, receptor type, M | | | | |
| | | | (PTPRM), mRNA cr: gi-18860903/// | | | | |
| | | | [Human_jongluer_201102.4361.C1] | | | | |
| | | | ; | | | | |
| | | | h_jongleur(identity):gi_3691957_gb | | | | |
| | | | _A1158775.1_A1158775 | | | | |
| 206 | 17 | AA002994_ESTs,_Moderately_simila | see Table 5 | [symbol] | [symbol] | [symbol] | |
| | | r_to_No_similarities_to_any_reported | | | | | |
| | | _proteins_[H.sapiens] | | | | | |
| 189 | 18 | AA727914_ESTs | see Table 5 | 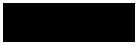 | [symbol] | [symbol] | |
| 101 | 19 | AA588976_ESTs | see Table 5 | [symbol] | 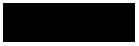 | [symbol] | |
| 199 | 20 | AA162022_ESTs | see Table 5 | [symbol] | [symbol] | [symbol] | |
| 125 | 21 | AA073925_ESTs | see Table 5 | [symbol] | 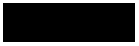 | [symbol] | |
| 326 | 22 | AA608457_DNA_polymerase_epsilon | gi\|5453925\|ret\|NM_006231.1] Homo | [symbol] | [symbol] | [symbol] | |
| | | | sapiens polymerase (DNA directed), | | | | |
| | | | epsilon (POLE), mRNA | | | | |
| 165 | 23 | A1551677_ESTs | see Table 5 | [symbol] | [symbol] | [symbol] | |
| 273 | 24 | AA624604_Public_domain_EST | see Table 5 | [symbol] | 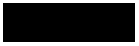 | [symbol] | |
| 249 | 25 | A1447967_ESTs | see Table 5 | [symbol] | 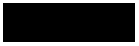 | [symbol] | |
| 107 | 26 | AA116829_RIKEN_cDNA_2610018 G03_gene | see Table 5 | [symbol] | 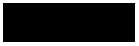 | [symbol] | |
| 129 | 27 | AA210488_ESTs | see Table 5 | 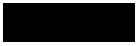 | 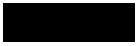 | [symbol] | |
| 191 | 29 | AA623587_ESTs | see Table 5 | [symbol] | [symbol] | [symbol] | |
| 131 | 29 | AA623060_RIKEN_cDNA_2410124 | see Table 5 | [symbol] | [symbol] | [symbol] | |
| | | L17_gene | | | | | |
| 281 | 30 | A1551943_ESTs,_Weakly_similar_to_{—} | see Table 5 | [symbol] | [symbol] | [symbol] | |
| | | LC2378_acetyl-CoA_C- | | | | | |
| | | acetyltransferase_[H.sapiens] | | | | | |
| 267 | 31 | AA470284_serine/threonine_kinase_5 | see Table 5 | [symbol] | [symbol] | [symbol] | |
| 126 | 32 | AA412879_RIKEN_cDNA_C3300060 08_gene | see Table 5 | 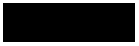 | 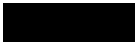 | [symbol] | |
| 106 | 33 | AA607983_zinc_finger_protein_101 | see Table 5 | [symbol] | 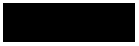 | [symbol] | |
| 250 | 34 | A1614738_RIKEN_cDNA_261030111 5_gene | see Table 5 | [symbol] | 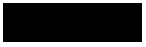 | [symbol] | |
| 196 | 35 | AA469648_growth_factor,_erv1_(S._cerevisiae)- like_faugmenter_of_liver_regeneratio n) | see Table 5 | [symbol] | 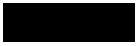 | [symbol] | |
| 287 | 36 | AA389264_ESTs | see Table 5 | [symbol] | [symbol] | [symbol] | |
| 128 | 37 | AA623175_ESTs | see Table 5 | [symbol] | [symbol] | [symbol] | |
| 171 | 38 | A1594170_ESTs | see Table 5 | [symbol] | [symbol] | [symbol] | |
| 213 | 39 | A1549643_RIKEN_cDNA_1300017N 15_gene | see Table 5 | [symbol] | 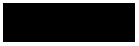 | [symbol] | |
| | | 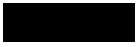 | | spl | panc | 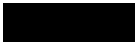 | |
| 75 | 1 | A1892379_aminolevulinic_acid_synth ase_1 | see Table 5 | [symbol] | [symbol] | 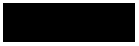 | |
| 27 | 2 | AA756672_thyroid_hormone_respons ive_SPOT14_homolog_(Rattus) | see Table 5 | [symbol] | [symbol] | 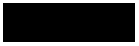 | |
| 327 | 3 | A1425345_hydroxysteroid_17- beta-dehydrogenase_9 | Homo sapiens 3-hydroxysteroid epimerase (RODH), mRNA cr: gi- 19743807/// [Human_jongleur_201102.4709.C1] | [symbol] | [symbol] | 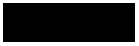 | |
| 74 | 4 | AA106125_cytochrome_P450,_2a4 | see Table 5 | [symbol] | [symbol] | 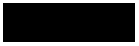 | |
| 73 | 5 | AA674177_cytochrome_P450, 2a4 | see Table 5 | [symbol] | [symbol] | 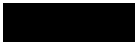 | |
| 64 | 6 | AA881013_cytochrome_P450,_2c37 | see Table 5 | [symbol] | [symbol] | 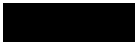 | |
| 329 | 7 | A1507919_alcohol_dehydrogenase_1, _complex | Human mRNA for liver alcohol dehydrogenase (EC 1.1.1.1) gamma 2 subunit from ADH3 locus si: gi- 28403/// [Human_jongleur_201102.c1.2404.si nglet] | [symbol] | [symbol] | 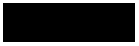 | |
| 65 | 5 | A1552452_solute_carrier_family_27_( fatty_acid_transporter)_member_5 | see Table 5 | 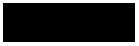 | [symbol] | 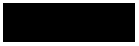 | |
| 330 | 9 | AA474336_synaptonenal_complex_p rotein_3 | orth Homo sapiens synaptonenal complex protein 3 (SYCP3), mRNA cr: gi-24233579/// [Human_jongluer_201102.4259.C3]; h_jongluer(strong homology):gi_2302563_gb_AA4743 361_AA474336 | [symbol] | [symbol] | 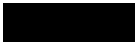 | |
| 331 | 20 | AA237317_solute_carrier_family_2_( facilitated_glucose_transporter),_mem ber_2 | Homo sapiens solute carrier family 2 (facilitated glucose transporter), member 2 (SLC2A2), mRNA cr: gi- 4557859/// [Human_jongluer_201102.11033.C1 ] | [symbol] | [symbol] | 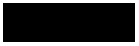 | |
| 319 | 11 | AA638765_metallothionein_1 | see above | [symbol] | [symbol] | 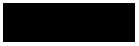 | |
| 56 | 12 | AA162217_pre-B-cell_colony- enhancing_factor | see Table 5 | [symbol] | [symbol] | 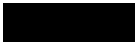 | |
| 245 | 13 | AA274023_apoptosis_inhibitory_6 | see Table 5 | [symbol] | [symbol] | 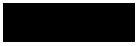 | |
| 243 | 14 | AA543982_interleukin_1_receptor_ty pe_1 | see Table 5 | [symbol] | [symbol] | 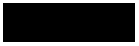 | |
| 333 | 15 | AA245848_hydroxysteroid_dehydrog enase-3,_delta<5>-3-beta | Homo sapiens hydroxy-delta-5- steroid dehydrogenase, 3 beta- and steroid delta-isomerase 2 (HSD3B2), mRNA | 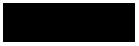 | [symbol] | 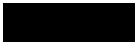 | |
| 76 | 16 | AA760070_Mus_musculus_triacylgly cerol_hydrolase_mRNA,_complete_cd s | see Table 5 | [symbol] | [symbol] | 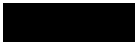 | |
| 334 | 17 | AA027607_hydroxysteroid_17- beta_dehydrogenase_2 | Homo sapiens hydroxysteroid (17- beta) dehydrogenase 2 (HSD17B2), mRNA cr: gi-4504502/// [Human_jongluer_201102.518.C1] | [symbol] | [symbol] | 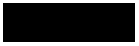 | |
| 28 | 18 | A1789976_fatty_acid_Coenzyme_A_li gase_long_chain_2 | see Table 5 | [symbol] | [symbol] | 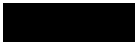 | |
| | | 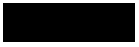 | | spl | 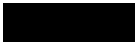 | liv | |
| 48 | 1 | AA619407_pancreatitis- associated_protein | see Table 5 | 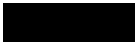 | 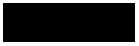 | [symbol] | |
| 355 | 2 | AA062401_TATA_box_binding_prot ein_(TBP)- associated_factor,_RNA_polymerase_H,_S,_150kD | orth Homo sapiens. Similar to TATA box binding protein (TBP)- associated factor, RNA polymerase H, S, 150kD, clone IMAGE:3636799, mRNA cr: gi- 15559581/// [Human_jongleur_201102.11703.C2 ]; h_jongleur(identity):gi_1556203 | | | | |
| #—gb _AA062401.1_AA062401 | [symbol] | 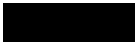 | [symbol] | | | | |
| 226 | 3 | AA210481_clusterin | see Table 5 | [symbol] | 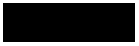 | [symbol] | |
| 14 | 4 | AA437717_ESTs,_Moderately_simila r_to_T34531_hypothetical_protein_D KFZp434P1215.1_(H.sapiens) | see Table 5 | [symbol] | 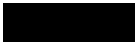 | [symbol] | |
| | | 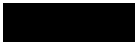 | | 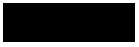 | panc | liv | |
| 116 | 1 | AA821884_amylase_2,_pancreatic | see Table 5 | 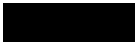 | [symbol] | [symbol] | |
| 336 | 2 | A1386058_serum_albumin_variant | Homo sapiens albumin (ALB), mRNA cr: gi-8392890/// [Human_jongleur_201102.252.C13] | 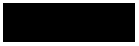 | [symbol] | 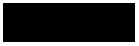 | |
| 248 | 3 | AA822106_ESTs,_Weakly_similar_to _C)8A_HUMAN_COMPLEMENT_COMPONENT_C8_ALPHA_CHAIN _PRECURSOR_[H.sapiens] | see Table 5 | 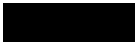 | [symbol] | 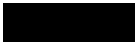 | |
| 337 | 4 | AA237793_rat_regenerating_islet- derived_mouse_homology_1 | Homo sapiens regenerating islet- derived 1 alpha (pancreatic stone protein, pancreatic thread protein) (REG1A), mRNA cr: gi-4506470/// [Human_jongleur_201102.658.C1] | 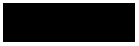 | [symbol] | [symbol] | |

| SEQ | | | | | | | | | | |
|---|---|---|---|---|---|---|---|---|---|---|
| ID | | | | | | | | PL-10B | PL-11B | PL-12B |
| NO | # | PL−8BBDEnor | PL−3BBDEnor | PL−5BBDEnor | PL−1BBDEnor | PL−7BBDEnor | PL−9BBDEnor | BDEnor | BDEnor | BDEnor |
| | | stom | int | col | br | lung | blad | kidn | pl/ut | 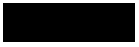 |
| 290 | 1 | [symbol] | 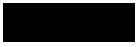 | 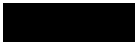 | [symbol] | [symbol] | [symbol] | [symbol] | [symbol] | 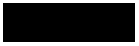 |
| 97 | 2 | [symbol] | [symbol] | [symbol] | [symbol] | [symbol] | [symbol] | [symbol] | [symbol] | 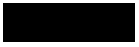 |
| 291 | [symbol] | [symbol] | 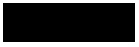 | [symbol] | [symbol] | [symbol] | [symbol] | [symbol] | 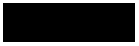 | |
| 292 | 4 | [symbol] | 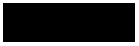 | [symbol] | [symbol] | [symbol] | [symbol] | [symbol] | [symbol] | 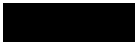 |
| 293 | 5 | [symbol] | [symbol] | [symbol] | [symbol] | [symbol] | 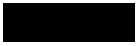 | [symbol] | [symbol] | 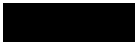 |
| 294 | 6 | [symbol] | [symbol] | [symbol] | [symbol] | [symbol] | [symbol] | [symbol] | 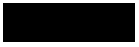 | 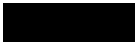 |
| | | stom | int | col | br | lung | blad | kidn | 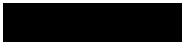 | mam |
| 127 | 1 | 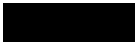 | [symbol] | [symbol] | 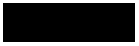 | 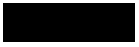 | 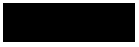 | 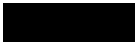 | 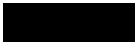 | 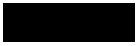 |
| 295 | 2 | 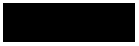 | 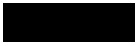 | [symbol] | [symbol] | 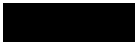 | 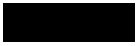 | 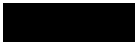 | 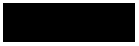 | 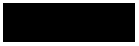 |
| | | stom | int | col | br | lung | blad | 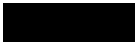 | pl/ut | mam |
| 91 | 1 | [symbol] | [symbol] | [symbol] | [symbol] | [symbol] | [symbol] | 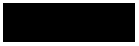 | [symbol] | [symbol] |
| 296 | 2 | [symbol] | [symbol] | [symbol] | [symbol] | [symbol] | [symbol] | 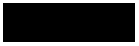 | [symbol] | [symbol] |
| 55 | 1 | [symbol] | [symbol] | [symbol] | [symbol] | [symbol] | [symbol] | 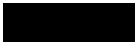 | [symbol] | [symbol] |
| | | stom | int | col | br | lung | 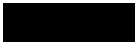 | kidn | pl/ut | mam |
| 96 | 1 | [symbol] | [symbol] | [symbol] | [symbol] | 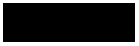 | 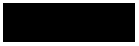 | [symbol] | [symbol] | [symbol] |
| 111 | 2 | [symbol] | [symbol] | [symbol] | [symbol] | 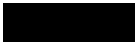 | 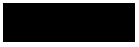 | [symbol] | [symbol] | [symbol] |
| 82 | 3 | [symbol] | [symbol] | [symbol] | [symbol] | 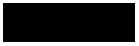 | 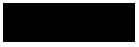 | [symbol] | [symbol] | [symbol] |
| 80 | 4 | [symbol] | [symbol] | [symbol] | [symbol] | 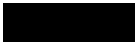 | 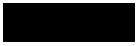 | [symbol] | [symbol] | [symbol] |
| 79 | 5 | 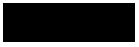 | [symbol] | [symbol] | [symbol] | [symbol] | 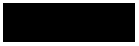 | [symbol] | 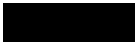 | 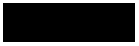 |
| 297 | 6 | [symbol] | [symbol] | [symbol] | [symbol] | 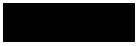 | 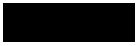 | [symbol] | [symbol] | [symbol] |
| 298 | 7 | [symbol] | [symbol] | 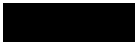 | [symbol] | [symbol] | 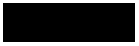 | [symbol] | [symbol] | [symbol] |
| | | stom | int | col | brain | 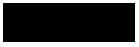 | blad | kidn | pl/ut | mam |
| 299 | 1 | [symbol] | [symbol] | [symbol] | [symbol] | 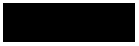 | [symbol] | [symbol] | [symbol] | [symbol] |
| 300 | 2 | [symbol] | [symbol] | [symbol] | [symbol] | 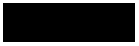 | [symbol] | [symbol] | [symbol] | [symbol] |
| 246 | 3 | [symbol] | [symbol] | [symbol] | [symbol] | 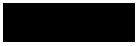 | 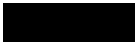 | [symbol] | [symbol] | [symbol] |
| 96 | 4 | [symbol] | [symbol] | [symbol] | [symbol] | 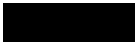 | 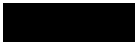 | [symbol] | [symbol] | [symbol] |
| 111 | 5 | [symbol] | [symbol] | [symbol] | [symbol] | 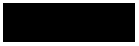 | 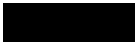 | [symbol] | [symbol] | [symbol] |
| 301 | 6 | [symbol] | [symbol] | [symbol] | [symbol] | 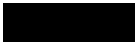 | 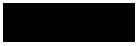 | [symbol] | [symbol] | [symbol] |
| 302 | 7 | [symbol] | [symbol] | [symbol] | [symbol] | 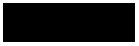 | [symbol] | [symbol] | [symbol] | [symbol] |
| | | stom | int | col | 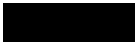 | lung | blad | kidn | pl/ut | mam |
| 218 | 1 | [symbol] | [symbol] | [symbol] | 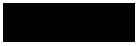 | [symbol] | [symbol] | [symbol] | [symbol] | [symbol] |
| 219 | 2 | [symbol] | [symbol] | [symbol] | 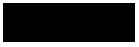 | [symbol] | [symbol] | 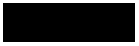 | [symbol] | [symbol] |
| 303 | 3 | [symbol] | [symbol] | [symbol] | 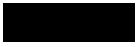 | [symbol] | [symbol] | [symbol] | [symbol] | [symbol] |
| 217 | 4 | [symbol] | [symbol] | [symbol] | 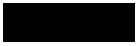 | [symbol] | [symbol] | 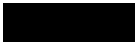 | [symbol] | [symbol] |
| 250 | 5 | 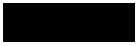 | [symbol] | [symbol] | 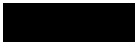 | [symbol] | [symbol] | 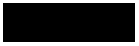 | [symbol] | 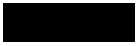 |
| 249 | 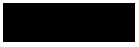 | [symbol] | 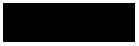 | 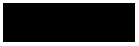 | 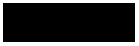 | 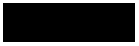 | 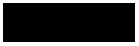 | 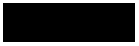 | | |
| 251 | 7 | 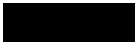 | 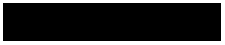 | 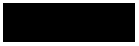 | 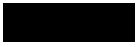 | 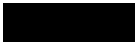 | [symbol] | 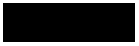 | 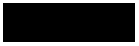 | |
| 222 | 8 | [symbol] | [symbol] | 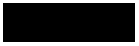 | [symbol] | [symbol] | [symbol] | [symbol] | [symbol] | |
| | | stom | int | 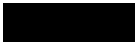 | br | lung | blad | kidn | pl/ut | mam |
| 304 | 1 | 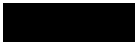 | 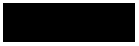 | [symbol] | [symbol] | [symbol] | [symbol] | [symbol] | [symbol] | |
| 182 | 2 | 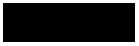 | 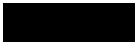 | [symbol] | [symbol] | [symbol] | [symbol] | [symbol] | [symbol] | |
| 305 | 3 | 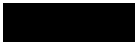 | 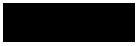 | [symbol] | [symbol] | [symbol] | [symbol] | [symbol] | [symbol] | |
| 306 | 4 | [symbol] | 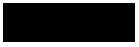 | [symbol] | [symbol] | [symbol] | [symbol] | [symbol] | [symbol] | |
| 290 | 5 | 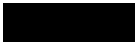 | 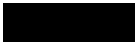 | [symbol] | [symbol] | [symbol] | [symbol] | [symbol] | 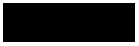 | |
| 7 | 6 | [symbol] | 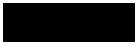 | [symbol] | [symbol] | [symbol] | [symbol] | [symbol] | [symbol] | |
| 308 | 7 | [symbol] | [symbol] | 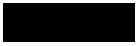 | [symbol] | [symbol] | [symbol] | [symbol] | [symbol] | [symbol] |
| 309 | 8 | [symbol] | [symbol] | 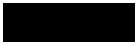 | [symbol] | [symbol] | [symbol] | [symbol] | [symbol] | [symbol] |
| 235 | 9 | [symbol] | [symbol] | 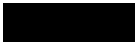 | [symbol] | [symbol] | [symbol] | [symbol] | [symbol] | [symbol] |
| 239 | 10 | [symbol] | [symbol] | 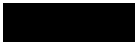 | [symbol] | [symbol] | [symbol] | [symbol] | [symbol] | [symbol] |
| 10 | [symbol] | [symbol] | 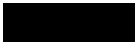 | [symbol] | [symbol] | [symbol] | [symbol] | [symbol] | [symbol] | |
| 310 | [symbol] | [symbol] | 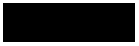 | [symbol] | [symbol] | [symbol] | [symbol] | [symbol] | [symbol] | |
| 36 | 13 | 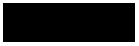 | [symbol] | 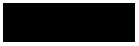 | [symbol] | [symbol] | [symbol] | [symbol] | 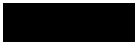 | 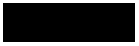 |
| 34 | 14 | [symbol] | [symbol] | 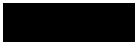 | [symbol] | [symbol] | [symbol] | [symbol] | [symbol] | [symbol] |
| 37 | 15 | 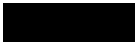 | 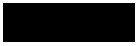 | 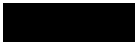 | [symbol] | [symbol] | [symbol] | [symbol] | 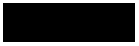 | [symbol] |
| 39 | 16 | [symbol] | 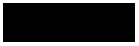 | 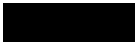 | [symbol] | [symbol] | [symbol] | [symbol] | [symbol] | [symbol] |
| 311 | 17 | [symbol] | [symbol] | 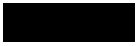 | [symbol] | [symbol] | [symbol] | [symbol] | [symbol] | [symbol] |
| 236 | 18 | [symbol] | [symbol] | 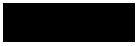 | [symbol] | [symbol] | [symbol] | [symbol] | [symbol] | [symbol] |
| 8 | 19 | [symbol] | [symbol] | 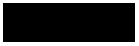 | [symbol] | [symbol] | [symbol] | [symbol] | [symbol] | [symbol] |
| 90 | 20 | 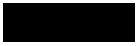 | [symbol] | 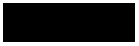 | [symbol] | [symbol] | [symbol] | [symbol] | [symbol] | [symbol] |
| 71 | 21 | [symbol] | 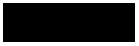 | 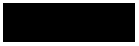 | [symbol] | [symbol] | [symbol] | [symbol] | [symbol] | [symbol] |
| | | stom | 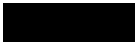 | col | br | lung | blad | kidn | pl/ut | mam |
| 48 | 1 | [symbol] | 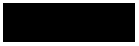 | [symbol] | [symbol] | [symbol] | [symbol] | [symbol] | [symbol] | [symbol] |
| 312 | 2 | [symbol] | 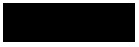 | 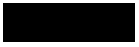 | [symbol] | [symbol] | [symbol] | [symbol] | [symbol] | [symbol] |
| 31 | 3 | [symbol] | 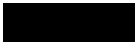 | [symbol] | [symbol] | [symbol] | [symbol] | [symbol] | [symbol] | [symbol] |
| 290 | 4 | [symbol] | 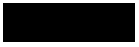 | 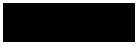 | [symbol] | [symbol] | [symbol] | [symbol] | [symbol] | 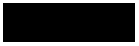 |
| 53 | 5 | [symbol] | 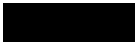 | [symbol] | [symbol] | [symbol] | [symbol] | [symbol] | [symbol] | [symbol] |
| 314 | 6 | [symbol] | 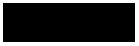 | 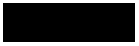 | [symbol] | [symbol] | [symbol] | 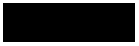 | [symbol] | [symbol] |
| 315 | 7 | [symbol] | 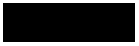 | [symbol] | [symbol] | [symbol] | [symbol] | [symbol] | [symbol] | [symbol] |
| 41 | 8 | 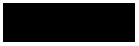 | 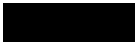 | 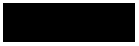 | [symbol] | [symbol] | [symbol] | [symbol] | [symbol] | 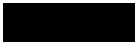 |
| 24 | 9 | [symbol] | 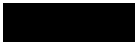 | [symbol] | [symbol] | [symbol] | [symbol] | [symbol] | [symbol] | [symbol] |
| 304 | 10 | [symbol] | 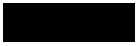 | 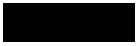 | [symbol] | [symbol] | [symbol] | [symbol] | [symbol] | [symbol] |
| 46 | 11 | [symbol] | 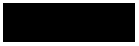 | [symbol] | [symbol] | [symbol] | [symbol] | [symbol] | [symbol] | [symbol] |
| 317 | 12 | [symbol] | 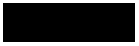 | 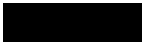 | [symbol] | [symbol] | [symbol] | [symbol] | [symbol] | [symbol] |
| 292 | 13 | [symbol] | 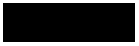 | [symbol] | [symbol] | [symbol] | [symbol] | [symbol] | [symbol] | 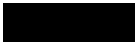 |
| 1 | 14 | [symbol] | 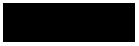 | 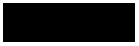 | [symbol] | [symbol] | [symbol] | [symbol] | [symbol] | [symbol] |
| 319 | 15 | [symbol] | 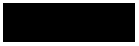 | 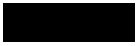 | [symbol] | [symbol] | [symbol] | 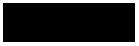 | [symbol] | [symbol] |
| 71 | 16 | [symbol] | 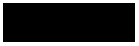 | 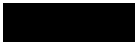 | [symbol] | [symbol] | [symbol] | [symbol] | [symbol] | [symbol] |
| | | 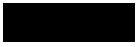 | int | col | brain | lung | blad | kidn | pl/ut | mam |
| 295 | 1 | 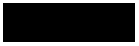 | 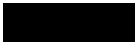 | [symbol] | [symbol] | 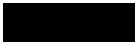 | 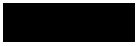 | 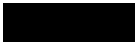 | 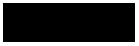 | 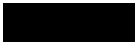 |
| 130 | 2 | 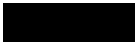 | 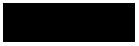 | [symbol] | [symbol] | 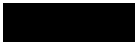 | 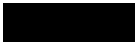 | [symbol] | 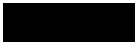 | 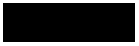 |
| 123 | 3 | 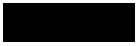 | [symbol] | [symbol] | 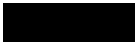 | [symbol] | 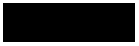 | [symbol] | [symbol] | 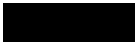 |
| 321 | 4 | 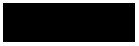 | 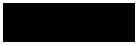 | [symbol] | [symbol] | 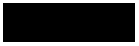 | 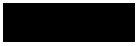 | [symbol] | 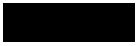 | 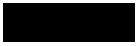 |
| 99 | 5 | 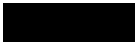 | 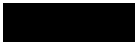 | [symbol] | [symbol] | 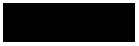 | 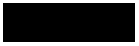 | 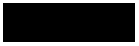 | 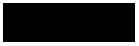 | 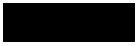 |
| 127 | 6 | 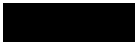 | [symbol] | [symbol] | 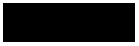 | 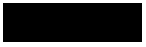 | 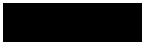 | 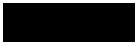 | 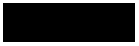 | 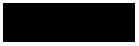 |
| 286 | 7 | 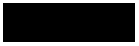 | [symbol] | [symbol] | 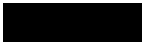 | [symbol] | [symbol] | [symbol] | [symbol] | [symbol] |
| 195 | 8 | 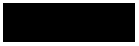 | 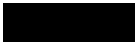 | [symbol] | 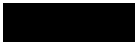 | 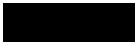 | 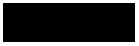 | [symbol] | 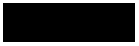 | 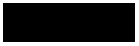 |
| 255 | 9 | 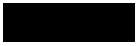 | 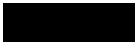 | [symbol] | 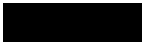 | 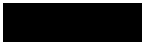 | 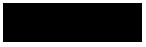 | [symbol] | 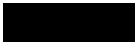 | 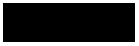 |
| 322 | 10 | 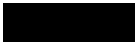 | [symbol] | [symbol] | [symbol] | 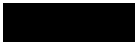 | 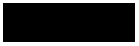 | 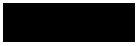 | 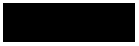 | 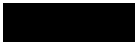 |
| 170 | 11 | 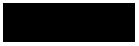 | 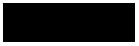 | [symbol] | [symbol] | 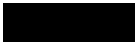 | [symbol] | [symbol] | [symbol] | 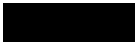 |
| 251 | 12 | 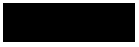 | 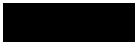 | 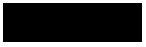 | 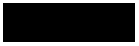 | 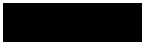 | 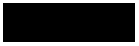 | [symbol] | 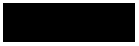 | 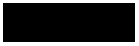 |
| 338 | 13 | 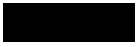 | 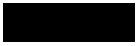 | [symbol] | [symbol] | [symbol] | [symbol] | [symbol] | 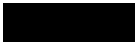 | [symbol] |
| 214 | 14 | 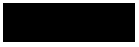 | [symbol] | [symbol] | [symbol] | [symbol] | 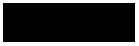 | [symbol] | 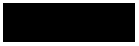 | [symbol] |
| 324 | 15 | 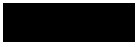 | [symbol] | [symbol] | [symbol] | [symbol] | [symbol] | 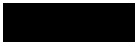 | [symbol] | 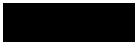 |
| 325 | 16 | 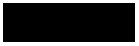 | [symbol] | [symbol] | [symbol] | [symbol] | [symbol] | [symbol] | [symbol] | [symbol] |
| 206 | 17 | 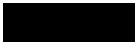 | [symbol] | [symbol] | 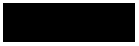 | [symbol] | [symbol] | [symbol] | [symbol] | [symbol] |
| 189 | 18 | 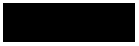 | [symbol] | [symbol] | 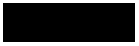 | 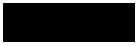 | 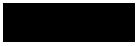 | [symbol] | 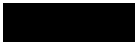 | [symbol] |
| 101 | 19 | 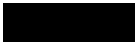 | 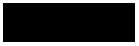 | [symbol] | 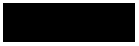 | 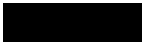 | 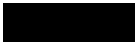 | [symbol] | 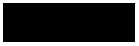 | 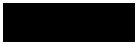 |
| 199 | 20 | 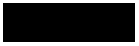 | 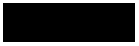 | [symbol] | [symbol] | 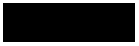 | 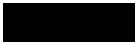 | [symbol] | 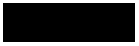 | 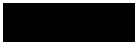 |
| 125 | 21 | 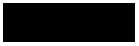 | 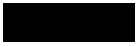 | [symbol] | [symbol] | 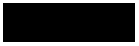 | 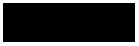 | [symbol] | 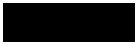 | 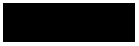 |
| 326 | 22 | 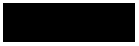 | 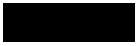 | [symbol] | [symbol] | 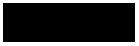 | [symbol] | [symbol] | 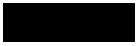 | [symbol] |
| 165 | 23 | 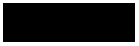 | 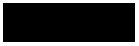 | [symbol] | 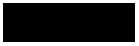 | [symbol] | [symbol] | [symbol] | [symbol] | 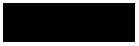 |
| 273 | 24 | 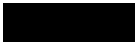 | [symbol] | [symbol] | [symbol] | [symbol] | [symbol] | [symbol] | [symbol] | [symbol] |
| 249 | 25 | 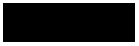 | 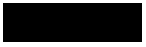 | [symbol] | 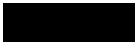 | 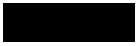 | 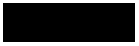 | 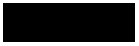 | 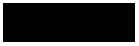 | 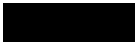 |
| 107 | 26 | 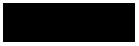 | 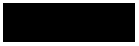 | [symbol] | [symbol] | [symbol] | [symbol] | [symbol] | [symbol] | [symbol] |
| 129 | 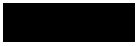 | [symbol] | [symbol] | [symbol] | 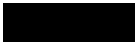 | 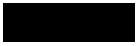 | [symbol] | 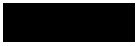 | 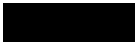 | |
| 191 | 28 | 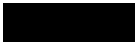 | [symbol] | [symbol] | [symbol] | 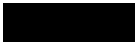 | 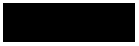 | [symbol] | [symbol] | 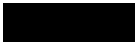 |
| 131 | 29 | 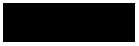 | [symbol] | [symbol] | 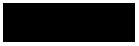 | 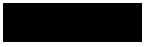 | 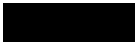 | [symbol] | 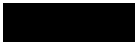 | 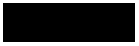 |
| 281 | 30 | 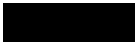 | [symbol] | [symbol] | [symbol] | 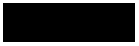 | 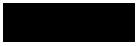 | 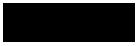 | 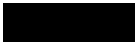 | 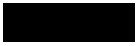 |
| 267 | 31 | 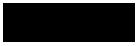 | [symbol] | [symbol] | [symbol] | [symbol] | [symbol] | [symbol] | [symbol] | [symbol] |
| 126 | 32 | 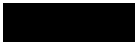 | [symbol] | [symbol] | [symbol] | 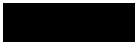 | 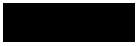 | [symbol] | 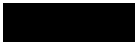 | 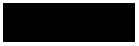 |
| 106 | 33 | 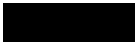 | 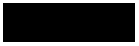 | [symbol] | 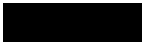 | [symbol] | [symbol] | [symbol] | [symbol] | 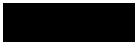 |
| 250 | 34 | 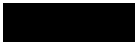 | [symbol] | [symbol] | 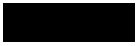 | [symbol] | [symbol] | 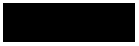 | [symbol] | 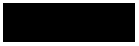 |
| 196 | 35 | 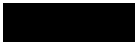 | 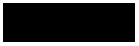 | [symbol] | [symbol] | [symbol] | [symbol] | [symbol] | [symbol] | [symbol] |
| 287 | 36 | 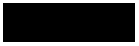 | [symbol] | [symbol] | [symbol] | [symbol] | [symbol] | 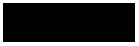 | 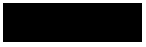 | [symbol] |
| 128 | 37 | 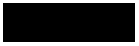 | 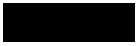 | [symbol] | [symbol] | 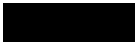 | 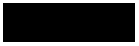 | 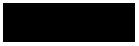 | 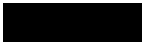 | 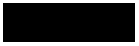 |
| 171 | 38 | 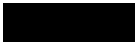 | [symbol] | [symbol] | [symbol] | [symbol] | [symbol] | [symbol] | [symbol] | [symbol] |
| 213 | 39 | 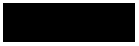 | [symbol] | [symbol] | [symbol] | 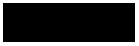 | 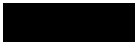 | [symbol] | 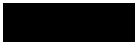 | 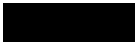 |
| | | stom | int | col | br | lung | blad | kidn | pl/ut | mam |
| 75 | 1 | [symbol] | [symbol] | [symbol] | [symbol] | [symbol] | [symbol] | 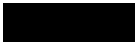 | [symbol] | [symbol] |
| 27 | 2 | [symbol] | [symbol] | [symbol] | [symbol] | [symbol] | [symbol] | [symbol] | [symbol] | [symbol] |
| 327 | 3 | [symbol] | [symbol] | [symbol] | [symbol] | [symbol] | [symbol] | [symbol] | [symbol] | [symbol] |
| 74 | 4 | [symbol] | [symbol] | [symbol] | [symbol] | [symbol] | [symbol] | 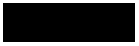 | [symbol] | [symbol] |
| 73 | 5 | [symbol] | [symbol] | 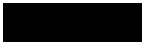 | [symbol] | [symbol] | [symbol] | 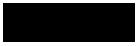 | [symbol] | [symbol] |
| 64 | 6 | [symbol] | 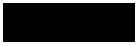 | [symbol] | [symbol] | [symbol] | [symbol] | [symbol] | [symbol] | [symbol] |
| 329 | 7 | [symbol] | [symbol] | [symbol] | [symbol] | [symbol] | [symbol] | [symbol] | [symbol] | [symbol] |
| 65 | 8 | [symbol] | [symbol] | [symbol] | [symbol] | [symbol] | [symbol] | [symbol] | [symbol] | [symbol] |
| 330 | 9 | [symbol] | [symbol] | 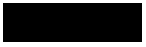 | [symbol] | [symbol] | [symbol] | [symbol] | [symbol] | [symbol] |
| 331 | 10 | [symbol] | [symbol] | 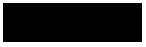 | [symbol] | [symbol] | [symbol] | [symbol] | [symbol] | [symbol] |
| 319 | 11 | [symbol] | 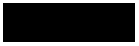 | 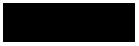 | [symbol] | [symbol] | [symbol] | 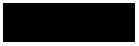 | [symbol] | [symbol] |
| 56 | 12 | [symbol] | [symbol] | [symbol] | [symbol] | [symbol] | [symbol] | [symbol] | [symbol] | [symbol] |
| 245 | 13 | [symbol] | [symbol] | [symbol] | [symbol] | [symbol] | [symbol] | [symbol] | [symbol] | [symbol] |
| 243 | 14 | [symbol] | [symbol] | [symbol] | [symbol] | [symbol] | [symbol] | [symbol] | [symbol] | [symbol] |
| 333 | 15 | [symbol] | [symbol] | [symbol] | [symbol] | [symbol] | [symbol] | [symbol] | [symbol] | [symbol] |
| 76 | 16 | [symbol] | [symbol] | [symbol] | [symbol] | [symbol] | [symbol] | [symbol] | [symbol] | [symbol] |
| 334 | 17 | [symbol] | 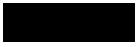 | [symbol] | [symbol] | [symbol] | [symbol] | [symbol] | [symbol] | [symbol] |
| 28 | 18 | [symbol] | [symbol] | [symbol] | [symbol] | [symbol] | [symbol] | [symbol] | [symbol] | [symbol] |
| | | stom | int | col | br | lung | blad | kidn | pl/ut | mam |
| 48 | 1 | [symbol] | 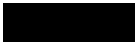 | [symbol] | [symbol] | [symbol] | [symbol] | [symbol] | [symbol] | [symbol] |
| 335 | 2 | [symbol] | [symbol] | [symbol] | [symbol] | [symbol] | [symbol] | [symbol] | [symbol] | [symbol] |
| 226 | 3 | [symbol] | [symbol] | [symbol] | [symbol] | [symbol] | [symbol] | [symbol] | [symbol] | [symbol] |
| 14 | 4 | [symbol] | [symbol] | [symbol] | [symbol] | [symbol] | [symbol] | [symbol] | [symbol] | [symbol] |
| | | stom | int | col | br | lung | blad | kidn | pl/ut | mam |
| 116 | 1 | [symbol] | [symbol] | [symbol] | 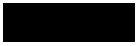 | [symbol] | [symbol] | [symbol] | [symbol] | [symbol] |
| 336 | 2 | [symbol] | [symbol] | 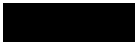 | [symbol] | [symbol] | [symbol] | [symbol] | [symbol] | [symbol] |
| 248 | 3 | [symbol] | [symbol] | 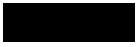 | [symbol] | [symbol] | [symbol] | [symbol] | [symbol] | [symbol] |
| 337 | 4 | [symbol] | [symbol] | [symbol] | [symbol] | [symbol] | [symbol] | [symbol] | [symbol] | [symbol] |

1. A method of diagnosing a cancer in a subject comprising determining, in a sample from the subject, the level of at least one polypeptide, wherein a higher level of the polypeptide compared to the level of the polypeptide in a subject free of cancer is indicative of cancer, and wherein the polypeptide is selected from the group consisting of:
a) polypeptides encoded by the polynucleotides listed in Table 5; and
b) polypeptides which are at least 70% homologous to the polypeptides of a).

2. The method of claim 1 wherein the sample is taken from a bodily fluid.

3. The method of claim 2 wherein the bodily fluid is selected from the group of fluid consisting of blood, lymph fluid, ascites, serous fluid, pleural effusion, sputum, cerebrospinal fluid, lacrimal fluid; synovial fluid, saliva, stool, sperm and urine.

4. The method of claim I wherein the level of the polypeptide is determined by a method selected from the group consisting of immunohistochemistry, western blotting, ELISA, antibody microarray hybridization and targeted molecular imaging.

5. A method of diagnosing a cancer in a subject comprising determining, in a sample from the subject, the level of at least one polypeptide, wherein a higher level of the polypeptide compared to the level of the polypeptide in a subject free of cancer is indicative of cancer, and wherein the polypeptide is selected from the group consisting of:
a) polypeptides encoded by the polynucleotides listed in Table 6; and
b) polypeptides which are at least 70% homologous to the polypeptides of a).

6. The method of claim 5 wherein the sample is a tissue sample.

7. The method of claim 6 wherein the tissue is selected from the group of tissue consisting of brain, lung, liver, spleen, kidney, pancreas, intestine, colon, mammary gland or breast, stomach, prostate, bladder, placenta and uterus.

8. The method of claim 5 wherein the level of the polypeptide is determined by a method selected from the group consisting of immunohistochemistry, western blotting, ELISA and targeted molecular imaging.

9. A method of diagnosing a cancer in a subject comprising determining, in a sample from the subject, the level of at least one polypeptide-encoding polylucleotide, wherein a higher level of the polynucleotide compared to the level of the polynucleotide in a subject free of cancer is indicative of cancer, and wherein the polynucleotide is selected from the group consisting of:
a) the polynucleotides listed in Table 6;
b) polynucleotides having sequences that differ from the polynucleotides in (a), without changing the polypeptide encoded thereby; and
c) polynucleotides which are at least 70% homologous to the polynucleotides of (a).

10. The method of claim 9 wherein the sample is a tissue sample.

11. The method of claim 10 wherein the tissue is selected from the group of tissue consisting of brain, lung, liver, spleen, kidney, pancreas, intestine, colon, mammary gland or breast, stomach, prostate, bladder, placenta and uterus.

12. The method of claim 9 wherein the sample is a is taken from a bodily fluid.

13. The method of claim 12 wherein the bodily fluid is selected from the group of fluid consisting of blood, lymph fluid, ascites, serous fluid, pleural effusion, sputum, cerebrospinal fluid, lacrimal fluid, synovial fluid, saliva, stool, sperm and urine.

14. The method of claim 9 wherein the level of the polynucleotide is determined by a method selected from: RT-PCR analysis, in-situ hybridization, polynucleotide microarray and Northern blotting.

15. A method of measuring the responsiveness of a subject to a cancer treatment comprising determining the level of at least one polypeptide in a sample taken from the subject before treatment, and comparing it with the level of said polypeptide in a sample taken from the subject after treatment, a decrease in said level indicating responsiveness of said subject to the cancer treatment, wherein the polypeptide is selected from the group consisting of:
a) polypeptides encoded by the polynucleotides listed in Table 5 and Table 6; and
b) polypeptides which are at least 70% homologous to the polypeptides of a).

16. The method of claim 15 wherein the sample is taken from a bodily fluid.

17. The method of claim 16 wherein the bodily fluid is selected from the group of fluid consisting of blood, lymph fluid, ascites, serous fluid, pleural effusion, sputum, cerebrospinal fluid, lacrimal fluid, synovial fluid, saliva, stool, sperm and urine.

18. The method of claim 15 wherein the level of the polypeptide is determined by a method selected from the group consisting of Western blotting, ELISA and targeted molecular imaging.

19. A method of measuring the responsiveness of a subject to a cancer treatment comprising determining the level of at least one polypeptide-encoding polynucleotide in a sample taken from the subject before treatment, and comparing it with the level of said polynucleotide in a sample taken from the subject after treatment, a decrease in said level indicating responsiveness of said subject to the cancer treatment, wherein the polynucleotide is selected from the group consisting of:
a) the polynucleotides listed in Table 6;
b) polynucleotides having sequences that differ from the polynucleotides in a), without changing the polypeptide encoded thereby; and
c) polynucleotides which are at least 70% homologous to the polynucleotides of a).

20. The method of claim 19 wherein said sample is blood or bone marrow cells.

21. The method of claim 19 wherein the level of the polynucleotide is determined by RT-PCR analysis.

22. The method of claim 15 or 19 wherein the treatment is administration of a chemotherapeutic drug.

23. The method of claim 15 or 19 wherein the treatment is radiotherapy.

24. The method of any one of claims 1, 5 or 9 wherein a change in the level of the polynucleotide or polypeptide as compared with the normal level is indicative of an abnormality in a tumor suppressor gene or a biological pathway in which a tumor suppressor gene is involved.

25. The method of claim 24 wherein the tumor suppressor gene is selected from the tumor suppressor group consisting of p53, Rb1 and PTEN.

26. The method of any one of claims 1, 5 or 9 wherein a change in the level of the polynucleotide or polypeptide as compared with the normal level is indicative of the effectiveness of a drug that modulates the activity of a tumor suppressor gene.

27. The method of claim 26 wherein the tumor suppressor gene is selected from the tumor suppressor group consisting of p53, Rb1 and PTEN.

28. A method of identifying a diagnostic marker for a cancer comprising:
a) obtaining a first cell from a first cell type of said cancer, said cell comprising a defective tumor suppressor expression;
b) obtaining a second cell of the first cell type, wherein said second cell comprises a wild-type tumor suppressor expression;
c) identifying genes having an increased level of expression in the first cell as compared to the second cell; and
d) selecting at least one gene of step c) as a diagnostic marker for the cancer.

29. The method of claim 28 further comprising:
a) obtaining a first cell from a second cell type of said cancer, said cell comprising a defective tumor suppressor expression;
b) obtaining a second cell of the second cell type, wherein said second cell comprises a wild-type tumor suppressor expression;
c) identifying genes having an increased level of expression in the first cell of the second cell type as compared to the second cell of the second cell type;
d) comparing the genes having an increased expression in the first cell type with the genes having an increased expression in the second cell type;
e) identifying genes having an increased expression in the first cell type but not in the second cell type; and
f) selecting at least one gene of step (e) as a diagnostic marker of a cancer of the first cell type.

30. The method of claim 28 or 29 wherein the tumor suppressor gene is selected from the tumor suppressor group consisting of p53, Rb1 and PTEN.

31. The method of claim 28 wherein the identification of step c) is performed using a microarray.

32. The method of claim 29 wherein the identification of step e) is performed using a microarray.

33. The method of claim 29 wherein the identification of step c) and e) are both performed using a microarray.

34. A method for screening for compounds that modulate the activity of a tumor suppressor gene comprising:
a) obtaining a cell comprising a defective tumor suppressor expression;
b) measuring the level of expression of a marker of Table 5 or 6 in the cell;
c) contacting the cell with a test compound; and
d) measuring the expression of the marker of step b) after the contacting step c), wherein a change in the level of expression after said contacting step as compared to the level of expression before said contacting step is indicative of the ability of the compound to modulate the activity of the tumor suppressor gene.

35. The method of claim 34 wherein the change in the level of expression in step c) is a reduction in the level of expression.

36. Use of compounds identified according to the method of claim 35 in the treatment of cancer or as anti-cancer drugs.
